(19) United States
(12) Patent Application Publication
(10) Pub. No.: US 2021/0188930 A1
(43) Pub. Date: Jun. 24, 2021

(54) PANDA AS NOVEL THERAPEUTIC

(71) Applicant: RUI JIN HOSPITAL, SHANGHAI JIAO TONG UNIVERSITY SCHOOL OF MEDICINE, Shanghai (CN)

(72) Inventors: Min LU, Shanghai (CN); Jiale WU, Shanghai (CN); Huaxin SONG, Shanghai (CN)

(21) Appl. No.: 16/959,906

(22) PCT Filed: Apr. 28, 2018
(86) PCT No.: PCT/CN2018/085190

§ 371 Date: Jul. 2, 2020

Related U.S. Application Data
(63) Continuation-in-part of application No. PCT/CN2018/070051, filed on Jan. 2, 2018.

(51) Int. Cl. C07K 14/47; G01N 33/68; A61K 45/06; G01N 33/574

(52) U.S. Cl. CPC C07K 14/4746; G01N 33/6848; G01N 2500/00; G01N 33/5748; G01N 2333/4748; A61K 45/06

(57) ABSTRACT

Disclosed herein is a novel p53 complex and a collection of compounds that can tightly associate with p53 to efficiently rescue wildtype p53 structure and function, and the methods of making and using the complex and the compounds, including for diagnosis, prognosis, and treatment of p53 related disorders such as cancer and aging.

### 1. TECHNICAL FIELD

Various biochemical complexes, drug candidates and methods of making and using the complexes and drug candidates, with a wide range of medical and therapeutic applications, including cancer therapy, cosmetic, research, and industrial applications are disclosed herein.

### 2. BACKGROUND

Various drug candidates and methods of for treating p53 related disorders, such as cancer have been proposed. Because these drug candidates and methods are not optimal, there is a need in the field for improved drug candidate and methods.

### 3. SUMMARY

Applicant has described herein a novel p53 AND Agent complex (“PANDA”); a collection of compounds with useful characteristics and can tightly associate with the PANDA Pocket (each compound a “PANDA Agent”); a pocket on p53 that interacts with PANDA Agent to form a PANDA (as used herein “PANDA Pocket” when PANDA Agent is not bound or “PANDA Core” when PANDA Agent is bound); three cysteine residues on p53 that are important in the formation of various PANDA and PANDA Cores, namely the cysteines are the amino acid corresponding to wtp53 positions cysteine 124 (“C124”), cysteine 135 (“C135”), and cysteine 141 (“C141”) (each a “PANDA Cysteine” and together a “PANDA Triad”); methods of making and using PANDA and/or PANDA Core, including in the diagnosis, prognosis, and treatment of p53 related disorders such as cancer and aging; and methods of using PANDA Agents, including in the diagnosis, prognosis, and treatment of p53 related disorders such as cancer and aging.

In certain embodiments, the PANDA Core is a tertiary structure formed on a p53 comprising of a PANDA Pocket, a PANDA Agent, and at least one tight association between the PANDA Pocket and the PANDA Agent. In a preferred embodiment, the PANDA Pocket is a region consisting essentially of an area of about 7 Å from a properly folded PANDA Cysteine, and includes, all amino acids adjacent to one or more properly folded PANDA Cysteine, all amino acids that contact with one or more properly folded PANDA Cysteine, and all PANDA Cysteines. In a preferred embodiment, the PANDA Agent is a composition of matter that has one or more useful characteristics. Examples of such useful characteristics of PANDA Agent include (a) can cause a substantial increase in the population of properly folded p53, preferably the increase is at least about 3 times more than the increase caused by PRIMA-1, more preferably the increase is at least about 5 times more than the increase caused by PRIMA-1, further preferably the increase is at least about 10 times more than the increase caused by PRIMA-1, further preferably the increase is at least about 100 times more than the increase caused by PRIMA-1; (b) can cause a substantial improvement in the transcription function of p53, preferably the improvement is at least about 3 times more than the improvement caused by PRIMA-1; more preferably the improvement is at least about 5 times more than the improvement caused by PRIMA-1, further preferably the improvement is at least about 10 times more than the improvement caused by PRIMA-1, further preferably the improvement is at least about 100 times than the improvement caused by PRIMA-1; and (c) can cause a substantial enhancement of stabilization of p53 as measured by, for example, an increase p53 Tₘ, preferably the enhancement is at least about 3 times more than the enhancement caused by PRIMA-1, more preferably the improvement is at least about 5 times more than the improvement caused by PRIMA-1, further preferably the improvement is at least about 10 times more than the improvement caused by PRIMA-1, further preferably the improvement is at least about 100 times than the improvement caused by PRIMA-1. In a preferred embodiment, a PANDA Agent has two or more useful characteristics. In a more preferred embodiment, a PANDA Agent has three or more useful characteristics.

In certain embodiments, the PANDA Pocket consists essentially of the PANDA Triad and the amino acids corresponding to wtp53 positions S116, C275, R273, Y234, V122, T123, T125, Y126, M133, F134, Q136, L137, K139, T140, P142, V143, L114, H115, G117, T118, A119, K120, S121, A138, I232, H233, N235, Y236, M237, C238, N239, F270, E271, V272, V274, A276, C277, P278, G279, R280, D281, and R282. In certain preferred embodiments, the PANDA Pocket is arranged essentially as in FIG. 14 left panel, FIG. 14 right panel, and/or FIG. 18.

A preferred p53 is any wildtype p53 (“wtp53”), any mutated p53 (“mp53”), all natural and artificial forms of wtp53 and mp53, and any combinations thereof. Preferred examples of wtp53 include p53α, p53β, p53γ, A40p53α, A40p53β, A40p53γ, and any acceptable variants, such as those with one or more single nucleotide polymorphism (“SNP”). Exemplar sequences of wtp53 human wtp53 isoforms as show in Section 7.25.

A preferred mp53 has at least one mutation on p53, including any single amino acid mutation. Preferably, the mutation alters and/or partially alters the structure and/or function of p53 Preferred examples of mp53 include one or more mutations at R175, G245, R248, R249, R273, R282, C176, H179, Y220, P278, V143, I232, and F270. Exemplar mp53 mutations include R175H, G245D/S, R248Q/W, R249S, R273C/H, R282W, C176F, H179R, Y220C, P278S, V143A, I232T, and F270C.

A preferred artificial p53 includes any artificially engineered p53. Preferred examples of an artificially engineered p53 include a p53 fusion protein, a p53 fragment, a p53 peptide, a p53-derived fusion macromolecule, a p53 recombinant protein, a p53 with second-site suppressor mutation (“SSSM”), and a super p53.

In certain embodiments, the tight association formed by PANDA Agent and PANDA Pocket can be a bond, covalent bond, a non-covalent bond (such as a hydrogen bond), and a combination thereof. In certain embodiments, the tight association is formed between PANDA Agent and one or more PANDA Cysteines, preferably two or more PANDA Cysteines, and more preferably all three PANDA Cysteines.

In certain embodiments, the PANDA Agent can regulate the level of one or more p53 target gene. Exemplar target genes include Apaf1, Bax, Fas, Dr5, mir-34, Noxa, TP53AIP1, Perp, Pidd, Pig3, Puma, Siva, YWHAZ, Btg2, Cdknla, Gadd45a, mir-34a, mir-34b/34c, Pri3, Ptprv, Reprimo, Pai1, Pml, Ddb2, Ercc5, Fancc, Gadd45a, Ku86, Mgmt, Mlh1, Msh2, P53r2, Polk, Xpc, Adora2b, Aldh4, Gamt, GIs2, Gpx1, Lpin1, Parkin, Prkab1, Prkab2, Pten, Sco1, Sesn1, Sesn2, Tigar, Tp53inp1, Tsc2, Atg10, Atg2b, Atg4a, Atg4c, Atg7, Ctsd, Ddit4, Dram1, Foxo3, Laptm4a, Lkb1, Pik3r3, Prkag2, Puma, Tpp1, Tsc2, Ulk1, Ulk2, Uvrag, Vamp4, Vmp1, Bai1, Cx3d1, Icam1, Irf5, Irf9, Isg15, Maspin, Mcp1, Ncf2, Pai1, Tir1-Tlr10, Tsp1, Ulbp1, Ulbp2, mir-34a, mir-200c, mir-145, mir-34a, mir-34b/34c, and Notch1.

In certain embodiments, the tight association formed by PANDA Agent and PANDA Core substantially stabilizes p53. Preferably, the tight association increases the Tₘ of p53 by at least about 0.5° C., more preferably by at least about 1° C., further preferably by at least about 2° C., further preferably by at least about 5° C., further preferably by at least about 8° C.

In certain embodiments, the tight association formed by PANDA Agent and PANDA Core increases the population of properly folded p53 by at least about 3 times, preferably by about 5 times, more preferably by about 10 times, and further preferably by about 100 times. In preferred embodiments, the increase is measured by a PAb1620 immunoprecipitation assay.

In certain embodiments, the PANDA Agent includes one or more PANDA Pocket-binding group (“R”) capable of binding one or more amino acids on PANDA Pocket, preferably one or more cysteine, more preferably two or more cysteines, further preferably more than three cysteines, further preferably from about three cysteines to about 12 cysteines. R is preferred to include metallic group(s), metalloid group(s), and other group(s) capable of binding to PANDA Pocket such as Michael acceptor(s) and thiol group(s). R is further preferred to include one or more arsenic, antimony, and bismuth, including any analogue(s) thereof, and any combinations thereof. Exemplar R(s) include compounds containing a 3-valence and/or 5-valence arsenic atom, a 3-valence and/or 5-valence antimony atom, a 3-valence and/or 5-valence bismuth atom, and/or a combination thereof. Exemplar PANDA Agents include Table 1-Table 6, which Applicant has predicted to efficiently bind to PANDA Cysteines and efficiently rescue p53 in vitro, in vivo and/or in situ. More exemplar PANDA Agents include of As₂O₃, As₂O₅, KAsO₂, NaAs₂, HAsNa₂O₄, HAsK₂O₄, AsF₃, AsCl₃, AsBr₃, AsI₃, AsAc₃, As(OC₂H₅)₃, As(OCH₃)₃, As₂(SO₄)₃, (CH₃CO₂)₃As, C₆H₄K₂O₁₂As₂.xH₂O, HOC₆H₄COOAsO, [O₂CCH₂C(OH)(CO₂)CH₂CO₂]As, Sb₂O₃, Sb₂O₅, KSbO₂, NaSbO₂, HSbNa₂O₄, HSbK2O4, SbF3, SbCl3, SbBr3, SbI3, SbAc3, Sb(OC2H5)3, Sb(OCH3)3, Sb2(SO4)3, (CH3CO2)₃Sb, C₈H₄K₂O₁₂Sb₂.xH₂O, HOC₆H₄COOSbO, [O₂CCH₂C(OH)(CO₂)CH₂CO₂]Sb, Bi₂O₃, Bi₂O₅, KBiO₂, NaBiO₂, HBiNa₂O₄, HBiK₂O₄, BiF₃, BiCl₃, BiBr₃, BiI₃, BiAc₃, Bi(OC₂H₅)₃, Bi(OCH₃)₃, Bi₂(SO₄)₃, (CH₃CO₂)₃Bi, C₆H₄K₂O₁₂Bi₂.xH₂O, HOC₈H₄COOBiO, C₁₆H₁₈As₂N₄O₂ (NSC92909), C₁₃H₁₄As₂O (NSC48300), C₁₀H₁₃NO₈Sb (NSC31660), C₆H₁₂NaO₈Sb⁺ (NSC15609), C₁₃H₂₁NaO₉Sb⁺ (NSC15623), and a combination thereof. Further exemplar PANDA Agents include Table 7, which Applicant has confirmed by experiment to show strong degree of structural rescue and transcriptional activity rescue.

In certain embodiments, the PANDA Core is produced by a reaction between the PANDA Pocket and the PANDA Agent. Preferably, the reaction is preferably mediated by an As, Sb, and/or Bi group oxidizing one or more thiol groups of PANDA Cysteines (PANDA Cysteines lose between one to three hydrogens) and the As, Sb, and/or Bi group of PANDA Agent is reduced (PANDA Agent loses oxygen). In certain embodiments, the PANDA Agents are the reduzate formed from having tightly associated with p53. In certain embodiments, the PANDA Agent is an arsenic atom, an antimony atom, a bismuth atom, any analogue thereof, or a combination thereof.

An exemplar PANDA Core is substantially similar to the corresponding amino acids on the three-dimensional structure of FIG. 14 left panel (Appendix A), FIG. 14 right panel (Appendix B) and/or FIG. 18. In certain preferred embodiments, the PANDA Core has about a 3.00 RMSD and/or 0.50 TM-score in jCE Circular Permutation comparison to the corresponding amino acids on the three-dimensional structure of FIG. 14 left panel (Appendix A), FIG. 14 right panel (Appendix B) and/or FIG. 18, preferably about a 2.00 RMSD and/or 0.75 TM-score fit, further preferably about a 1.00 RMSD and/or 0.90 TM-score fit. In certain preferred embodiments, the PANDA Core corresponds to the amino acids on the three-dimensional structure of FIG. 14 left panel (Appendix A), FIG. 14 right panel (Appendix B) and/or FIG. 18. In certain preferred embodiments, the amino acids corresponding to wtp53 amino acids 114-126, 133-143, 232-239, and 270-282 on PANDA Core is substantially similar to the corresponding location FIG. 14 left panel (Appendix A), FIG. 14 right panel (Appendix B) and/or FIG. 18.

In certain embodiments, the structure of PANDA is substantially similar to the three-dimensional structure of FIG. 14 left panel (Appendix A), FIG. 14 right panel (Appendix B), and/or FIG. 18. In certain preferred embodiments, the PANDA has about a 3.00 RMSD and/or 0.50 TM-score in jCE Circular Permutation comparison to the three-dimensional structure of FIG. 14 left panel (Appendix A), FIG. 14 right panel (Appendix B) and/or FIG. 18, preferably about a 2.00 RMSD and/or 0.75 TM-score fit, further preferably about a 1.00 RMSD and/or 0.90 TM-score fit. In certain preferred embodiments, the PANDA corresponds to the three-dimensional structure of FIG. 14 left panel (Appendix A), FIG. 14 right panel (Appendix B) and/or FIG. 18. In certain preferred embodiments, the amino acids corresponding to wtp53 amino acids 114-126, 133-143, 232-239, and 270-282 on PANDA is substantially similar to the corresponding location FIG. 14 left panel (Appendix A), FIG. 14 right panel (Appendix B) and/or FIG. 18.

In certain embodiments, formed PANDA can be purified and isolated using any conventional methods, including any methods disclosed in this Application, such as by immunoprecipitation using PAb1620.

In certain preferred embodiments, as compared to when the PANDA Agent is not bound, formed PANDA has gained one or more wtp53 structure, preferably a DNA binding structure; has gained one or more wtp53 function, preferably a transcription function; and/or has lost and/or diminishes one or more mp53 function, preferably an oncogenic function. The wildtype function can be gained in vitro and/or in vivo. Exemplar wildtype function gained can be at the molecule-level, such as association to nucleic adds, transcriptional activation or repression of target genes, association to wtp53 or mp53 partners, dissociation to wtp53 or mp53 partners, and reception to post-translational modification; at the cell-level, such as, responsiveness to stresses such as nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, invasion and metastasis; and at the organism-level, such as delay or prevention of cancer relapse, increase of cancer treatment efficacy, increase of response ratio to cancer treatment, regulation of development, senescence, longevity, immunological processes, and aging. The mp53 functions can be lost, impaired and/or abrogated in vitro and/or in vivo. Exemplar mp53 function lost can include any functions, such as oncogenic functions that promotes cancer cell metastasis, genomic instability, invasion, migration, scattering, angiogenesis, stem cell expansion, survival, proliferation, tissue remodelling, resistance to therapy, and mitogenic defects.

In certain preferred embodiments, the formed PANDA can gain and/or lose the ability to upregulate or downregulate one or more p53 downstream targets, at an RNA level and/or protein level, in a biological system, preferably by 3 times, more preferably by 5 times, further preferably by 10-100 times.

In certain preferred embodiments, the PANDA Agent any of the preceding claims having the ability to treat a p53-relevant disease in a subject with mp53 and/or without functional p53, wherein the disease is a cancer, a tumor, a consequence of aging, a developmental disease, accelerated aging, an immunological disease, or a combination thereof.

In certain preferred embodiments, the formed PANDA has the ability to suppress tumors, preferably least to a level that is statistically significant; more preferably having the ability to strongly suppress tumors at a level that is statistically significant. In certain preferred embodiments, the formed PANDA has the ability to regulate cell growth or tumor growth preferably to at least about 10% of the wtp53 level, further preferably at least about 100% of the wtp53 level, further preferably exceeding about 100% of the wtp53 level.

In certain preferred embodiments, PANDA or PANDA Core can be made by combining one or more PANDA Agent to a p53, preferably a mp53 with at least one mutation on p53, including a single amino acid mutation. Preferably, the mutation alters and/or partially alters the structure and/or function of p53. Preferred examples of mp53 include one or more mutations at R175, G245, R248, R249, R273, R282, C176, H179, Y220, P278, V143, I232, and F270. Exemplar mp53 mutations include R175H, G245D/S, R248Q/W, R249S, R273C/H, R282W, C176F, H179R, Y220C, P278S, V143A, I232T, and F270C.

In certain preferred embodiments, the PANDA Agent can rescue one or more wtp53 structure, preferably a DNA binding structure; rescue one or more wtp53 function, preferably a transcription function, eliminating and/or diminishes one or more mp53 function, preferably an oncogenic function.

In certain preferred embodiments, one or more wtp53 structure, preferably a DNA binding structure can be rescued by combining one or more PANDA Agent to a p53 to form PANDA, preferably a mp53 with at least one mutation on p53, including a single amino acid mutation. Preferably, the mutation alters and/or partially alters the structure and/or function of p53. Preferred examples of mp53 include one or more mutations at R175, G245, R248, R249, R273, R282, C176, H179, Y220, P278, V143, I232, and F270. Exemplar mp53 mutations include R175H, G245D/S, R248Q/W, R249S, R273C/H, R282W, C176F, H179R, Y220C, P278S, V143A, I232T, and F270C.

In certain preferred embodiments, one or more wtp53 function, preferably a preferably a transcription function can be rescued by combining one or more PANDA Agent to a p53 to form PANDA, preferably a mp53 with at least one mutation on p53, including a single amino acid mutation. Preferably, the mutation alters and/or partially alters the structure and/or function of p53. Preferred examples of mp53 include one or more mutations at R175, G245, R248, R249, R273, R282, C176, H179, Y220, P278, V143, I232, and F270. Exemplar mp53 mutations include R175H, G245D/S, R248Q/W, R249S, R273C/H, R282W, C176F, H179R, Y220C, P278S, V143A, I232T, and F270C.

In certain preferred embodiments, one or more mp53 function, preferably an oncogenic function, can be eliminated and/or diminished by combining one or more PANDA Agent to a p53 to form PANDA, preferably a mp53 with at least one mutation on p53, including a single amino acid mutation. Preferably, the mutation alters and/or partially alters the structure and/or function of p53. Preferred examples of mp53 include one or more mutations at R175, G245, R248, R249, R273, R282, C176, H179, Y220, P278, V143, I232, and F270. Exemplar mp53 mutations include R175H, G245D/S, R248Q/W, R249S, R273C/H, R282W, C176F, H179R, Y220C, P278S, V143A, I232T, and F270C.

In certain preferred embodiments, one or more wtp53 structure, preferably a DNA binding structure can be rescued by adding a PANDA and/or a PANDA Agent to a cell, preferably a human cell, and/or a subject, preferably a human subject.

In certain preferred embodiments, one or more wtp53 function, preferably a preferably a transcription function can be rescued by adding a PANDA and/or a PANDA Agent to a cell, preferably a human cell, and/or a subject, preferably a human subject.

In certain preferred embodiments, one or more mp53 function, preferably an oncogenic function, can be eliminated and/or diminished by adding a PANDA and/or a PANDA Agent to a cell, preferably a human cell, and/or a subject, preferably a human subject.

Applicant discloses herein a method of turning on and off a wtp53 function of a mp53, the method comprising the steps:

(a) combining a first PANDA Agent with the mp53 to turn on the wtp53 function of a mp53; and
(b) adding a second compound that (i) removes the PANDA Agent from the mp53, such as, British Anti-Lewisite (BAL), succimer (DMSA), Unithiol (DMPS), and/or a combination thereof; (ii) inhibits expression of p53, such as doxycycline in engineered cells or subjects, and/or (iii) turning off p53 expression, such as tamoxifen, in engineered cells or subjects.

Applicant discloses herein a method of using the PANDA or PANDA Core in vitro and/or in vivo to rescue one or more wtp53 structure, preferably a DNA binding structure; rescue one or more wtp53 function, preferably a transcription function; eliminate and/or diminishes one or more mp53 function, preferably an oncogenic function, the method comprising the step of adding a PANDA or PANDA Agent to a cell, preferably a human cell, and/or subject, preferably a human subject.

Applicant discloses herein group of PANDA Agents having the ability to treat a disease in a subject with mp53, the disease is preferably cancer.

Applicant discloses herein a method of treating a p53 related disorder in a subject in need thereof such as cancer, tumour, aging, developmental diseases, accelerated aging, immunological diseases, and/or a combination thereof. The method comprises the step of administering to a subject an effective amount of a therapeutic, wherein the therapeutic is (a) one or more PANDA Agents or (b) one or more PANDA or PANDA Core. In a preferred embodiment, the therapeutic is administered in combination with one or more additional therapeutic, preferably any known therapeutic effective at treating cancer and/or DNA damaging agent.

Applicant further discloses a highly efficient personalized method of treatment for a p53 related disorder in a subject in need thereof. The method comprises the steps of: (a) obtaining a p53 DNA sample from the subject; (b) sequencing the p53 DNA sample; (c) determining whether the p53 of the subject is rescuable and identifying one or more PANDA Agent and/or a combination of PANDA Agent that is most appropriate to rescue the p53 in the subject; and (d) administering an effective amount of the PANDA Agent and/or the combination of PANDA Agent to the subject; wherein step (c) includes the step(s) (i) determining in silico whether the sequence of the p53 DNA sample is comparable to a to a database of rescuable p53s and identifying the corresponding PANDA Agent(s) and/or combination of PANDA Agents most appropriate to rescue the p53 using the database; and/or (ii) determining in vitro and/or in vivo whether the p53 of the subject can be rescued by screening it against a panel of PANDA Agents.

Applicant further discloses a method of identifying PANDA or PANDA Core. The method comprising the step of: using an antibody specific for properly folded PANDA, such as PAb1620, PAb246, and/or PAb240, to perform immunoprecipitation; measuring increase of molecular weight by mass spectroscopy; measuring whether transcriptional activity is restored in a luciferase assay; measuring the mRNA and protein levels of p53 targets; co-crystalizing to construct 3-D structure; and/or measuring increase of Tₘ.

Applicant discloses herein a collection of PANDA Agents having the ability to regulate the levels of p53 targets in a biological system expressing a mp53 or lacking any functional p53. Applicant further discloses a method of controlling one or more protein and/or RNA regulated by p53 and/or PANDA, the method comprising the step of administering a regulator to a biological system, wherein the regulator is selected from a group consisting of:

(i) one or more PANDA Agent(s);

(ii) one or more PANDA or PANDA Core;

(iii) one or more compound that removes the PANDA Agent from the p53;

(iv) one or more mp53;

(v) one or more compound that removes PANDA, including an anti-p53 antibody, a doxycycline, and anti-PANDA antibody; and

(vi) a combination thereof.

Applicant discloses herein a collection of PANDA Agents having the ability to suppress tumors in a biological system, preferably a system that expresses a mp53. Applicant further discloses a method of suppressing tumors, the method comprising the step(s) of administering to a subject in need thereof an effective amount of a therapeutic, wherein the suppressor is selected from a group consisting of:

(i) one or more PANDA Agent(s); and

(ii) one or more PANDA and/or PANDA Core.

In a preferred embodiment, the suppressor is administered in combination with one or more additional suppressor, preferably any known suppressor effective at suppressing tumor growth and/or DNA damaging agent.

Applicant discloses herein a collection of PANDA Agents having the ability to regulate cell growth or tumor growth in a biological system, preferably a system that expresses a mp53. Applicant further discloses a method of regulating cell growth or tumor growth, the method comprising the step of administering to a subject in need thereof an effective amount of a regulator, wherein the regulator is selected from a group consisting of:

(i) one or more PANDA Agent(s); and (ii) one or more PANDA and/or PANDA Core. In a preferred embodiment, the regulator is administered in combination with one or more additional regulator, preferably any known regulator effective at slowing cell growth and/or DNA damaging agent.

Applicant discloses herein a method of diagnosing a p53 related disorder, such as cancer, tumor, aging, developmental diseases, accelerated aging, immunological diseases, or a combination thereof, in a subject in need thereof. The diagnosis method comprising the steps of administering to the subject an effective amount of a therapeutic, and detecting whether PANDA or PANDA Core is formed wherein the therapeutic is selected from a group consisting of:

(i) one or more PANDA Agent(s); and

(ii) one or more PANDA and/or PANDA Core.

In a preferred embodiment, the diagnosing method includes a treatment step wherein the therapeutic is administered in combination with one or more additional therapeutic, such as one or more additional PANDA Agent(s) and/or any other known therapeutic effective at treating cancer and/or DNA damaging agent, to effectively treat the p53 related disorder in the subject.

In certain embodiments, the PANDA Agent is not CP-31398; PRIMA-1; PRIMA-1-MET, SCH529074, Zinc; stictic acid, p53R3; methylene quinuclidinone; STIMA-1; 3-methylene-2-norbornanone; MIRA-1; MIRA-2; MIRA-3; NSC319725; NSC319726; SCH529074; PARP-PI3K; 5,50-(2,5-furandiyl)bis-2-thiophenemethanol; MPK-09; Zn-curc or curcumin-based Zn(III)-complex; P53R3; a (2-benzofuranyl)-quinazoline derivative; a nucleolipid derivative of 5-fluorouridine; a derivative of 2-aminoacetophenone hydrochloride; PK083; PK5174; or PK7088; and other previously identified mp53 rescue compound.

In certain embodiments, the PANDA Agent can be formulated in a pharmaceutical composition suitable for treating a subject with a p53 related disorder. A pharmaceutical composition will typically contain a pharmaceutically acceptable carrier. Although oral administration of a compound is the preferred route of administration, other means of administration such as nasal, topical or rectal administration, or by injection or inhalation, are also contemplated. Depending on the intended mode of administration, the pharmaceutical compositions may be in the form of solid, semi-solid, or liquid dosage forms, such as, for example, tablets, suppositories, pills, capsules, powders, liquids, suspensions, ointments, or lotions, preferably in unit dosage form suitable for single administration of a precise dosage. One skilled in this art may further formulate the compound in an appropriate manner, and in accordance with accepted practices, such as those disclosed in Remington's Pharmaceutical Sciences, Gennaro, Ed., Mack Publishing Co., Easton, Pa. 1990.

In certain embodiments, a carrier can be any and all solvents, dispersion media, vehicles, coatings, diluents, antibacterial and antifungal agents, isotonic and absorption delaying agents, buffers, carrier solutions, suspensions, colloids, and the like. A pharmaceutical carrier can include, liposomes, albumin microspheres, soluble synthetic polymers, DNA complexes, protein-drug conjugates, carrier erythrocytes, and any other substance that is incorporated to improve the delivery and the effectiveness of drugs. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

In certain embodiments, therapies used for the treatment of p53 related disorder, such as cancer, include, surgery, chemotherapy, and radiation therapy. Experimental therapies include, but are not limited to, expression of wildtype p53 in tumors based on viral or viral like particle based delivery vectors.

In certain embodiments, a p53 cancer therapeutic include, general chemotherapeutics. Examples of general chemotherapeutics include, but are not limited to, Avastin, Rituxan, Herceptin, Taxol, and Gleevec.

In certain embodiments, “a person in need of” can refer to an individual who has a p53 related disorder, such as a cancer, wherein the cancer expresses a mutated version of p53. In some embodiments, the p53 mutant is susceptible to PANDA Agent.

In certain embodiments, PANDA Agents can be formulated in a pharmaceutically acceptable salt. The pharmaceutically acceptable salt can be an ionizable drug that has been combined with a counter-ion to form a neutral complex. Converting a drug into a salt through this process can increase its chemical stability, render the complex easier to administer, and allow manipulation of the agent's pharmacokinetic profile (Patel, et al., 2009).

In certain embodiments, the PANDA Agent and PANDA have the following features:

(1) PANDA Agent ATO binds directly to p53 to form PANDA, in a process that changes p53 structure, including folds the mp53;

(2) PANDA Agent mediated PANDA formation can take place both in vitro and in vivo, including in humans;

(3) PANDA is remarkably similar to wtp53 in both structure and function;

(4) PANDA Agent ATO folds the structure of Structural mp53s with a striking high efficiency so that the structure of PANDA is remarkably similar to that of wtp53;

(5) PANDA Agent ATO rescues the transcriptional activity of Structural mp53 through PANDA with a strikingly high efficiency;

(6) PANDA Agent ATO inhibits growth of mp53 expressing cells in vitro and in vivo through PANDA;

(7) mp53 expressing cells treated with PANDA Agent ATO or cells containing PANDA actively responds to DNA-damaging treatment;

(8) PANDA Agent ATO is highly effective and specific to mp53 and an effective mp53 rescue agent;

(9) PANDA Agent ATO and PANDA can directly combat a wide range of cancers, including acute myeloid leukemia (“AML”) and myelodysplastic syndromes (“MDS”); and

(10) cancer patients, including patients with AML and MDS begin to show remarkable response to anti-cancer treatments when first treated with ATO or PANDA.

In certain embodiments, the PANDA Agents, such as those containing elemental arsenic, through the formation of PANDA, can wide-broad and efficiently rescue mp53s. For example, As₂O₃ and its analogues can rescue the most frequent mp53s in varying degrees. These mp53s include but are not limited to: six hotspot mp53s (mp53s with mutations on either R175, G245, R249, or R282 (commonly considered as structural hotspot mp53s), mp53s with mutations on either R248 or R273 (commonly considered as contacting hotspot mp53s), and mp53s with mutations on C176, H179, Y220, or P278, V143, F270, or 1232.

In certain embodiments, PANDA Agents has the potential to bind multiple cysteines and can selectively inhibit Structural mp53 expressing cells via promoting mp53 folding.

In certain embodiments, PANDA Agents transforms cancer-promoting mp53 to tumor suppressive PANDA and have significant advantages over existing therapeutic strategies such as by reintroducing wtp53 or promoting degradation/inactivation of endogenous mp53 in the patient. The PANDA Agent mediated mp53 rescue through PANDA, high rescue efficiency and mp53 selectivity are the two superior characteristics over previously-reported compounds. In certain embodiments, the PANDA Agent ATO can provide a near complete rescue of p53-R175H, from a level equivalent to about 1% of that of wtp53 to about 97% of that of wtp53 using the robust PAb1620 (also for PAb246) IP assay. In certain embodiments, the PANDA Agent ATO also provides a near complete rescue of the transcriptional activity of p53-G245S and p53-R282W on some pro-apoptotic targets, from a level equivalent to about 4% of that of wtp53 to about 80% of that of wtp53, using a standard luciferase reporter assay. Applicant has robustly reproduced these superior results, as compared to existing compounds, in numerous contexts and know no existing compound that can rescue the structure or transcriptional activity of a hotspot mp53 by a level equivalent to about 5% of that of wtp53 in our assays.

In certain embodiments, the PANDA Agent ATO and PANDA can selectively target Structural mp53 with strikingly high efficiency. In addition, Contracting mp53s can also be rescued with moderate efficiency. For example, Applicant found a wide range of Structural mp53s, including a large percentage of hotspot mp53s, can be efficiently rescued by the PANDA Agent ATO through the formation of PANDA. In addition, Applicant also found that the Contacting mp53s can be rescued by ATO through PANDA with a limited efficiency. This remarkable property is not only superior but is conceptually different from most of the reported compounds, including CP-31398 (Foster et al., 1999), PRIMA-1 (Bykov et al., 2002), SCH529074 (Demma et al., 2010), Zinc (Puca et al., 2011), stictic acid (Wassman et al., 2013), p53R3 (Weinmann et al., 2008), and others that are reported to be able to rescue both types of mp53.

Our discovery further shows that PANDA Agent ATO can be used for a wide range of ATO-responsive cancers in clinical trials. It is preferred that patient recruitment follow a specific, highly precise, recruitment prerequisite, in order to achieve maximum efficacy. While ATO was approved by FDA to treat acute promyelocytic leukemia (APL), a subtype of leukemia. Although ATO has been intensively trialed, aiming to broaden its application to non-APL cancer types over the past two decades, it has not yet been approved for this purpose. This is largely attributed to a failure to reveal an ATO-affecting cancer spectrum. Indeed, no mp53 dependency can be observed in the sensitivity profile of ATO on the NCI60 cell panel simply by differentiating lines into a mp53 group and a wtp53 group. By further separating ATO-rescuable mp53s out of the mp53s, we have successfully revealed the key elements for ATO and PANDA dependent response. The ATO-affecting cancer spectrum we discovered is considerably wide, covering an estimated amount of 15%-30% cancer cases. For example, we have identified at least 4 of the 6 hotspot mp53s and a large number of non-hotspot mp53s to be efficiently rescuable by ATO and PANDA. Indeed, in the earliest ATO clinical trial in China in 1971 (n>1000 patients), ATO showed an efficacy in treating many cancer types including colorectal, esophageal, liver, and particularly APL cancers (Zhang et al., 2001; Zhu et al., 2002).

While ATO and PANDA can be used to treat a wide range of cancers, it is preferable that ATO be precisely administrated to patients harboring ATO rescuable mp53, as demonstrated by some of the tests described in this application. It is known that different missense mutations will confer different activities to mp53 (Freed-Pastor and Prives, 2012), which can lead to different treatment outcomes in patients harboring different mp53s. Accordingly, others like us advocate tailoring treatments to the types of mp53 mutations present rather than whether mp53 or wtp53 is present (Muller and Vousden, 2013, 2014). Remarkably, our discoveries on the MDS patient-derived p53-S241F, p53-S241C as well as the other artificially generated p53 mutants on S241 support that ATO rescuing efficiency is determined not only by the p53 mutation site but also by the new residue generated. Based on the current promising outcomes observed in our small-scale AML/MDS trial, we have launched two large-scale multi-center prospective trials on AML/MDS patients (NCT03381781 and NCT03377725). In one trial, 300 MDS patients are being blindly recruited and trialed, aiming to confirm the dependency of ATO on p53 mutation status. In the other trial, approximately 1500-2000 AML patients are being recruited, the mp53-positive patients confirmed by sanger sequencing are being trialed to determine the efficacy of ATO in treating mp53-expressing non-APL leukemia.

Despite many rescue principles that have been proposed, the void of an atom-level rationale on how to pharmacologically rescue mp53 has blocked the advances of cancer research for too long (Bullock and Fersht, 2001; Joerger and Fersht, 2007; Joerger and Fersht, 2016). This void has significantly hindered scientists from identifying an efficient and selective mp53 rescue compound (Bullock and Fersht, 2001; Joerger and Fersht, 2007; Joerger and Fersht, 2016). To rationally design and screen mp53 stabilizer is particularly challenging because of the pockets on the p53 for a mp53 stabilizer to bind have not been known (Joerger and Fersht, 2016).

Applicant has further describe a rational 4C Screening method. Using this method, Applicant has identified compounds that covalently crosslinked to cysteine-pairs on mp53. Applicant predicts that covalently crosslinking cysteines may be robust enough to immobilize the local region, neutralize the flexibility caused by the nearby mutations and stabilize p53 globally.

Using our 4C screening, we successfully identified at least two arsenic-containing compounds that can act as rescuers for a wide spectrum of mp53s. When we explored the properties of these arsenic compounds, we identified an unexpected and deeply buried PANDA Pocket that the stabilizer binds to. In doing so, we provided an atom-level MOA of how a wide-spectrum of mp53s can be stabilized by a compound.

In certain embodiments, the PANDA Pocket plays a key role in stabilizing mp53 globally. We discovered that a large number of reported SSSMs is located on the PANDA Pocket. In addition, our rationally designed SSSMs, also located on the PANDA Pocket function to stabilize it. Our rescue mechanism and highly druggable PANDA Pocket can now explain why the previously reported Michael acceptor-containing compounds have barely detectable mp53 rescue efficiency (Joerger and Fersht, 2016; Muller and Vousden, 2014). Computer modelling suggested that many of these compounds bind to C124 (Wassman et al., 2013), one of the cysteines of the key PANDA Triad, however remaining to be determined experimentally (Joerger and Fersht, 2016) Because these Michael acceptor containing compounds contact the rims of the PANDA Triad, they can only very weakly stabilize PANDA Pocket, thus rescue mp53 with limited efficiency.

Arsenic's selectivity for cysteines of PANDA Triad in Structural mp53s are particularly attracting. So far, many compounds including PRIMA-1, STIMA-1, MIRA-1, “compound 1”, PK11007, and ellipticine have a Michael acceptor group and are predicted to bind a single cysteine to function (Bauer et al., 2016; Joerger and Fersht, 2016; Wassman et al., 2013). Since p53 possesses of more than one exposed cysteines, these compounds may bind to many other undesired cysteine(s). Indeed, PRIMA-1 and “compound 1” have been reported to bind mp53 with a ratio high than 1:1 in vitro (Bauer et al., 2016; Lambert et al., 2009). These compounds can also have off-target tendencies to wtp53 or other cellular proteins with exposed cysteines.

Our current arsenic-containing compounds are conceptually different from any previously reported compounds due to its multiple cysteine binding potential, which may explain the selectivity for the PANDA Triad. Arsenic selectively binds to the inert cysteines on the PANDA Triad rather than cysteines that are more accessible (e.g.: C277 and C182) or other tri-cysteine dusters (e.g.: C176/C238/C242 in zinc region). This suggests that the PANDA Triad is unique and are arranged in a special pattern particularly receptive to arsenic.

We also discovered that although ATO binds to wtp53 and Contacting mp53 to significantly stabilize them and enhance their function, by far, Structural mp53s benefited the most from ATO binding. One reason is Structural mp53s are highly unstable.

As discussed in other Sections of this application, the Structural mp53 selectivity we discovered is also conceptually different from most of the reported compounds such as CP-31398 (Foster et al., 1999), PRIMA-1 (Bykov et al., 2002), SCH529074 (Demma et al., 2010), Zinc (Puca et al., 2011), stictic acid (Wassman et al., 2013), and p53R3 (Weinmann et al., 2008). Our observed Structural mp53s selectivity is also of particularly high clinical value because cysteine-binding compounds have been intensively debated (and often disputed) for their druggability, due their high potential for off-targeting (and thus toxicity) in cells. Indeed, others have identified that one of the major milestones to turn research on current mp53-rescuing compounds from its current proof-of-concept studies into clinical trials is to improve mp53 selectivity (Joerger and Fersht, 2016; Kaar et al., 2010). Compared to PRIMA-1 and its analogue, which is under phase II clinical trial (Bauer et al., 2016; Joerger and Fersht, 2016), and which increasingly have been suggested to target oxidative stress signaling components in cells, rather than mp53, our PANDA Agents are highly effective and specific towards p53.

The clear rescue MOA we revealed here and the druggable PANDA Pocket we identified here will enable us and others to perform ultra-large-scale Screening to greatly expand our arsenal against cancer and greatly accelerate our effort to beat cancer. As disclosed here, we identified a large number of arsenic, antimony, and/or bismuth containing compounds that can efficiently rescue mp53. We are excited that some of the identified arsenic analogues may be superior to the approved clinically approved ATO. For example, while Fowler's solution (KAsO₂) has significant side-effects and are not used in clinical settings any more in past decades, As₄S4 has been shown to be as effective as conventional intravenous ATO in treating APL patients while it can be conveniently orally administrated (Zhu et al., 2013). The additional class of Sb and Bi compounds we identified, including many organic compounds, are also of significant clinical value, because they are known to be less toxic in the body.

Finally, the organic As, Sb, and/or Bi compounds are particularly interesting. On one hand, the diversity of organic groups supplies millions of modification choices to generate an enhanced version of mp53 rescuer. For example, introducing a large organic group may have more profound influence on mp53's structure, facilitating identification of an efficient mp53 inhibitor. A direct mp53 inhibitor with a clear atom-level MOA is very attracting because existing mp53 inhibitors (HSP90 inhibitor, HDAC inhibitors, RETRA, ATRA etc.) do not target mp53 directly and yet some of them have diverse effects on many ubiquitous cellular pathways (Sabapathy and Lane, 2018).

Here, we describe that both inorganic and organic As, Sb, and/or Bi compounds are mp53 rescuers. In addition, we describe that As, Sb, and/or Bi compounds with potential to bind a cysteine or bi-cysteine pairs can also rescue mp53. Furthermore, we describe that As, Sb, and/or Bi compounds with three or more cysteine binding potential have even higher rescue efficiency, some at levels comparable to wtp53.

Since we have identified that PANDA Pocket is a switch that controls p53 stability, we predict that other compounds, in addition to compounds containing As, Sb, and/or Bi, that can bind to PANDA Pocket will have profound influence on p53 structure. These compounds may either rescue mp53 by restoring the wildtype (or functional) structure to rescue mp53, or inhibit mp53 by distorting mp53's oncogenic structures. While the former compounds can be developed into mp53 rescue agents, the latter compounds are also of huge value as mp53 inhibitors.

Using the 4C screen method, we discovered, for the first time, a number of PANDA Agents with the remarkable capability that can almost completely rescue the structure of a wide range of mp53s, including mp53-R175H, to that of the wildtype. We further identified at least 31 leading PANDA Agents (Table 7), including the clinical pharmaceutical compound arsenic trioxide (“ATO”). ATO is thus used as an example in the followed context. Previously, our colleagues have combined ATO with all-trans retinoic acid (“ATRA”) to efficiently target the cysteine-enriched promyelocytic leukaemia (“PML”) moiety of PML-RARα fusion protein (Zhang et al., 2010), making acute promyelocytic leukemia (“APL”) the only malignancy that can be definitively cured by a targeted therapy (Hu et al., 2009; Lo-Coco et al., 2013).

### 4. BRIEF DESCRIPTION OF THE DRAWINGS

FIG. 1. ‘4C’ screen overview.

FIG. 2. Plot graph shows the G150 (retrieved by CellMiner) of ATO and KAsO2 on the NCI60 cell panels. Struc.: cell lines expressing structural hotspot mp53 (R175, G245, R249, and R282); WT: cell lines expressing wtp53; Others: the remained cell lines.

FIG. 3. Schemed hydroxylation and cysteine reaction of ATO and KAsO₂.

FIG. 4. H1299 cells transfected with p53-R175H were treated with 1 μg/ml ATO or 0.1 μg/ml KAsO2 for 2 hr, and cells were lysed followed by immunoprecipitation (IP) using PAb1620 (upper panel) or PAb240 (middle panel). Immunoprecipitated p53 was immunoblotted. Lower panel, ATO and KAsO2 treated Trp53-R172H/R172H MEFs were lysed, followed by PAb246 IP. p53 was probed.

FIG. 5. Classification of mp53. Image shows the p53-DNA complex (PDB accession: 1TUP) generated by Pymol. The six p53 mutation hotspots are labelled as either gray solid spheres (function in contacting DNA: R248 and R273) or black solid spheres (function in maintaining p53 structure: R175, G245, R249, and R282). The 10 cysteines of p53 were labelled.

FIG. 6. Cell Selectivity. NCI60 cell lines were differentiated into two categories, lines containing structural hotspot mp53 and the remined lines.

FIG. 7. Compound analysis. Examples of multiple cysteines binding potential compounds, such as compounds containing two Michael acceptor groups, Sb metal, or two thiols.

FIG. 8. Protein Conformation. Cartoon figures show the locations of mutually exclusive PAb1620 epitope and PAb240 epitope, which exist on folded p53 and unfolded p53, respectively. PAb246 epitope specifically exist on folded mouse p53 and it does not overlap with the PAb1620.

FIG. 9. Plot graph shows the G150 (retrieved by CellMiner) of PRIMA-1 and NSC319726 on the NCI60 cell panels. Struc.: cell lines expressing structural hotspot mp53 (R175, G245, R249, and R282); Others: the remained cell lines.

FIG. 10 ATO greatly increases mp53 stability by increasing its Tₘ. Left panel, melting curve of the purified p53 core domain R249S(94-293) recorded via differential scanning fluorimetry in absence or presence of ATO. Right panel, ATO of different concentrations was incubated with 5 μM purified p53 core domain R249S(94-293) for overnight. The melting temperatures of p53 core were shown (mean±SD, n=3).

FIG. 11. For p53 folding assay, H1299 cells transfected with indicated p53 were treated with 1 μg/ml ATO for 2 hr, and cells were lysed followed by immunoprecipitation using PAb1620. Immunoprecipitated p53 was immunoblotted. Experiments are repeated twice. For p53 transcriptional activity assay, H1299 cells were co-transfected with indicated p53 and PUMA reporter for 24 hr, followed by treatment of 1 μg/ml ATO for 24 hr. Plot shows the ATO-mediated mp53 rescue profile, derived from p53 folding assay and transcriptional activity assay. X-axis: PAb1620 IP efficiency; Y-axis: PUMA luciferase report signal. Hollow cycles: without ATO treatment; solid cycles: with ATO treatment.

FIG. 12. the purified recombinant p53(94-293)-R249S were treated with either DMSO (left panel) or ATO (right panel) at 1:5 molar ratio for overnight, followed by MS analysis for molecular weight determination. Spectrum image shows the deconvoluted spectra of purified protein under native denaturing conditions.

FIG. 13. Upper panel, H1299 cells transfected with indicated mp53s were treated with 4 μg/ml Biotin-As for 2 hr, cells were lysed, followed by pull-down assay using streptavidin beads. p53 was probed. Lower panel, H1299 cells transfected with indicated amount of p53-R175H or wtp53 plasmid were treated with 4 μg/ml Biotin-As for 2 hr, cells were lysed, followed by pull-down assay using streptavidin beads. p53 was probed.

FIG. 14 Bacteria expressing p53(94-293)-R249S were incubated with AsI3, the PANDA complex (see also FIG. 18) was then purified for crystallization (Left panel). The p53(94-293)-R249S crystal was soaked with 2 mM EDTA and 2 mM ATO for 19 h (Right panel). The 3D structure of PANDA was generated by Pymol. The C124, C135, and C141 and bound arsenic atom are show.

FIG. 15 Arsenic atom passes through L1-S2-S3 pocket and enters the PANDA Triad. Left panel: existing mp53 rescue compounds enter L1-S2-S3 pocket only when it is open. Right panel: arsenic atom is smaller than any of the reported mp53 rescue compounds by one or two orders of magnitude (about 1/10- 1/100 size of reported compounds). It can freely enter into L1-S2-S3 pocket at any time, even when it is closed. In addition, Arsenic atom is so small that it can freely pass through L1-S2-S3 pocket and further enter into the PANDA Triad, an extremely small pocket that can only accommodate one atom. At PANDA Triad, arsenic atom functions as an efficient PANDA Agent.

FIG. 16 Schematic 3D structure of p53 (PDB accession: 1TUP) and PANDA generated by Pymol. Left panel, the six p53 mutation hotspots are shown as either gray solid spheres (function in contacting DNA: R248 and R273) or black solid spheres (function in maintaining p53 structure: R175, G245, R249, and R282). The PANDA Cysteines (C124, C135, and C141) were labelled. Middle panel, the six p53 mutation hotspots and DNA are selected for presenting. Right panel, imaged scheme of PANDA in which contacting residue R248 holds bamboo while the other contacting residue R282 eat bamboo. PANDA Pocket functions as the hind neck known to stabilize a panda cub when being grabbed by its mother.

FIG. 17 the purified recombinant p53(94-293)-R249S were treated with indicated compounds at 1:5 molar ratio for overnight, followed by MS analysis for molecular weight determination. Spectrum image shows the deconvoluted spectra of purified protein under native denaturing conditions.

FIG. 18 Upper panel, 3D structure of PANDA shown as ribbons. The PANDA Triad and arsenic atom are shown as spheres, the PANDA Pocket are shown in darker colour. Middle panel, 3D structure of PANDA shown as spheres. The PANDA Pocket are shown in darker colour. Lower panel, the residues of PANDA Pocket. The structure are organized.

FIG. 19 Left panel, H1299 cells were co-transfected with indicated p53 mutation on p53-G245S plasmid and either PUMA reporter or PIG3 reporter for 24 hr. Bar graph shows the transcriptional activity of p53-G245S with designated SSSMs (mean±SD, n=3). Right panel, the upwards arrows and downwards arrows show the locations of mutations tested in left panel. Upwards arrows (S116 and Q136): mutations rescue p53-G245S, Downwards arrows: mutations fail to rescue p53-G245S.

FIG. 20 ATO strongly promotes proper folding of the unfolded population of p53. Left panel shows H1299 cells transfected with wtp53 and mp53s were treated with 1 μg/ml ATO for 2 hr; cells were lysed followed by immunoprecipitation (IP) using PAb1620. Immunoprecipitated p53 was immunoblotted. Right graph shows the relative PAb1620 IP efficiency. The PAb1620 IP efficiency for wtp53 in the absence of ATO was set as 100%.

FIG. 21 ATO efficiently and properly folds mp53s. Left panel, H1299 cells transfected with p53-R175H were treated with indicated agents for overnight, cells were lysed followed by PAb1620 IP. Right graph shows the normalized change of PAb1620 IP efficiency compared with the one in DMSO group.

FIG. 22 ATO efficiently refolds mp53s. Detroit 562 cells expressing endogenous p53-R175H were pre-treated with CHX for indicated conditions. Cells were then treated with 1 μg/ml ATO for 2 hr, followed by PAb1620 IP. Cartoon figure schemes the equilibria of p53-R175H among properly folded, unfolded, and aggregated status.

FIG. 23 1stM1D ATO efficiently and properly folds mp53s. Saos-2 cells transfected with wtp53 and p53-R175H were treated with 0, 0.2, 0.5, and 1 μg/ml ATO for 24 hr. Cells were lysed in CHAPS buffer at 4° C. or 37° C. for 15 min, followed by non-denaturing PAGE and western blot.

FIG. 24 ATO efficiently and properly folds mp53s. H1299 cells transfected with wtp53 and indicated mp53s were treated with 0 or 1 μg/ml ATO for 2 hr, followed by PAB1620 IP.

FIG. 25 Upper left panel, H1299 cells expressing p53-R175H were treated with ATO under indicated conditions, followed by PAb1620 IP. Middle left panel: Trp53+/+ MEFs (treated with 10 μM Nutlin3 overnight to induce a high level of p53) and Trp53-R172H/R172H MEFs were treated with 1 μg/ml ATO for 2 hr, followed by PAb246 IP. p53 was probed with CM5 antibody. Lower left panel, H1299 cells expressing p53-R175H were treated with 1 μg/ml ATO for 2 hr, followed by PAb240 IP. Right Panels show cells expressing a variety of mp53s treated with ATO under indicated conditions, followed by PAb1620 IP (PAb246 for MEFs).

FIG. 26 H1299 cells transfected with p53-R175H were treated with indicated agents for overnight, cells were lysed followed by PAb1620 IP.

FIG. 27 Trp53+/+ MEFs (treated with 10 μM Nutlin3 overnight to induce a high level of p53) and Trp53-R172H/R172H MEFs were treated with ATO of indicated concentration for 2 hr, followed by PAb246 IP.

FIG. 28 Bacteria expressing IPTG-inducible GST-p53-R175H was cultured with IPTG and indicated compounds. Bacteria were lysed in NP40 buffer, followed by IP using PAb1620. GST-p53-R175H was immunoblotted by GST antibody.

FIG. 29 MCF7 cells expressing endogenous wtp53 were treated with CHX as indicated, p53 was probed.

FIG. 30 H1299 cells expressing p53-R175H were pre-treated with either DMSO or 50 μg/ml CHX for 0.5 hr, cells were then treated with ATO, followed by PAb1620 IP.

FIG. 31 Saos-2 cells transfected with wtp53 and p53-R175H were treated with ATO as indicated. Cells were lysed in M-PER buffer at 4° C., followed by non-denaturing PAGE and western blot.

FIG. 32 shows the p53-DNA complex (PDB accession: 1TUP) generated by Pymol. The 3 dusters of cysteines (C135/C141, C238/C242, C275/C277) and R175-neighboring C176 are shown.

FIG. 33 Arsenic directly binds to p53 to form PANDA. (A) H1299 cells transfected with indicated wtp53 or mp53s were treated with 4 μg/ml Biotin-As for 2 hr. Cells were lysed, followed by pull-down assay using streptavidin beads. p53 was probed. (B) Indicated cell lines were treated with 4 μg/ml Biotin-As for 2 hr. Cells were lysed, followed by pull-down assay using streptavidin beads. (C) Purified recombinant GST-p53-R175H were incubated with the indicated concentrations of Biotin-As or Biotin. The mixtures were divided into three aliquots and subjected to denaturing protein electrophoresis (SDS-PAGE), followed by Coomassie blue staining, p53 IB using DO1 antibody, or Biotin IB using anti-biotin antibody. (D) p53(62-292) (upper panel) and p53(91-292)-R175H (lower panel) were bacterially expressed with 100 μM ZnSO₄ and 10 μM ATO, respectively. After purification, recombinant proteins were subjected to MS analysis for Mw determination. Spectrum image shows the deconvoluted spectra of purified recombinant protein under native or denaturing conditions.

FIG. 34 Table shows the molecular weight (Mw) of purified recombinant p53(62-292) and p53(91-292)-R175H bacterially expressed with 100 μM ZnSO₄ and 50 μM ATO, respectively. Native and denaturing MS were applied to determine the Mw.

FIG. 35 Table summarizes the Arsenic content determined in the standard As₂O₃ solution and recombinant PANDA-R175H solution by inductively coupled plasma mass spectroscopy (ICP-MS).

FIG. 36 3 PANDA regains DNA-binding ability. H1299 cells expressing p53-R175H were treated with indicated agents overnight, and cells were lysed followed by pull-down assay using streptavidin beads in presence of 10 μM of biotinylated double-stranded DNA. p53-R175H was immunoblotted.

FIG. 37 PANDA regains transcriptional activity.

FIG. 38 PANDA regains DNA-binding ability and p53 transcriptional activity. Upper panel, H1299 cells expressing tet-off-regulated p53-R175H were pre-treated with/without doxycycline (“Dox”) for 48 hr, followed by 1 μg/ml ATO treatment for indicated duration. mRNA level of indicated p53 targets were determined by qPCR. Nutlin was used to treat wtp53 expressing HCT116, serving as control. Lower panel, BT549 cells expressing endogenous p53-R249S were treated with 1 μg/ml ATO for indicated duration. mRNA level of indicated p53 targets were determined by qPCR.

FIG. 39 PANDA upregulates the protein levels of p53 targets. H1299 cells expressing tet-off-regulated p53-R175H were pre-treated with/without doxycycline (Dox) for 48 hr, followed by 0.2 μg/ml ATO treatment for 48 hr. Protein levels of p53 targets were determined.

FIG. 40 Detroit 562 cells expressing endogenous p53-R175H were treated with ATO as indicated, followed by p53 immunoblotting.

FIG. 41 H1299 cells were co-transfected with p53-G245S and PIG3 reporter (left panel) or p53-R282W and PUMA reporter (right panel) for 24 hr, followed by treatment of indicated agents for 24 hr. Bar graph shows normalized changes in luciferase signals (mean±SD, n=3).

FIG. 42 HCT116 cells transfected with indicated mp53s were treated with 1 μg/ml ATO for 48 hr. Protein levels of PUMA was determined.

FIG. 43 CEM-C1 cells expressing endogenous p53-R175H were treated with ATO as indicated, followed by p53 immunoblotting.

FIG. 44 PANDA-mediated tumor suppression. H1299 cells expressing tet-off-regulated p53-R175H were pre-treated with/without doxycycline (DOX) for 48 hr, ATO was added for 48 hr, followed by MTT cell viability assay (left panel) and colony formation assay (right panel) (mean SD, n=3, *p<0.05).

FIG. 45 PANDA-mediated tumor suppression. Cell viability of 10 cell lines upon 48 hr ATO (left panel) or Nutlin (right panel) treatment (values show mean of three independent experiments).

FIG. 46 PANDA-mediated tumor suppression. Plot graph shows the G150 (retrieved by CellMiner) of ATO and Nutlin3 in the NCI60 cell panels (*p<0.05). Struc.: hotspot mutations on R175, G245, R249, and R282. Null: truncated p53, frame-shift p53 and null p53. Contact: hotspot mutations on R248 and R273. p53 status was compiled via the IARC TP53 database.

FIG. 47 PANDA-mediated tumor suppression. H1299 cells expressing tet-off-regulated p53-R175H were subcutaneously injected into flanks of nude mice. 5 mg/kg ATO was intraperitoneally injected for 6 consecutive d/week when the tumor area reached 0.1 cm (day 1). In DOX groups, drinking water contained 0.2 mg/ml DOX. Tumor size measurement was repeated every 3 d (left panel). Mice were sacrificed on day 28 and isolated tumors were weighed, followed by p53 IHC staining (right panel, bar=50 μm). Graphs show mean±SEM (*p<0.05, **p<0.01, ***p<0.001, n=4/group).

FIG. 48 PANDA-mediated tumor suppression. CEM-C1 cells were injected via tail vein into NOD/SCID mice. Peripheral blood (PB) samples were obtained from the mice retro-orbital sinus every 3 or 4 days from day 7 to day 26. After CEM-C1 (hCD45+) positive cells reached 0.1% in PB (day 23), mice were treated with vehicle (n=6) or ATO (5 mg/kg, n=7) intravenously for 6 consecutive days per week. Upper panel, the percentage of mCD45+ and hCD45+ cells in PB on day 16, 22, and 26. Lower panel, Mantel-Cox survival curves of vehicle or treated mice.

FIG. 49 MEFs expressing p53-R172H/R172H or null p53 were treated with ATO for 48 hr, followed by cell viability assay (left panel) and colony formation assay (right panel) (mean±SD, n=3, *p<0.05).

FIG. 50 Plot graph shows the G150 (retrieved by CellMiner) of PRIMA-1 and NSC319726 in the NCI60 cell panels. Struc.: hotspot mutations on R175, G245, R249, and R282. Null: truncated p53, frame-shift p53 and null p53. Contact: hotspot mutations on R248 and R273. p53 status was compiled via the IARC TP53 database.

FIG. 51 Cell viability of H1299 cells (null p53), H1299 cells expressing p53-R175H, or H1299 cells expressing wtp53 (DOX to induce expression of wtp53) upon Nutlin treatment in the absence (left panel) or presence (right panel) of 1 μg/ml ATO (mean±SD, n=3, *p<0.05).

FIG. 52 p53-R175H protein level determined in tumors isolated on day 28 as described in FIG. 47.

FIG. 53 Tumors are isolated on day 28 as described in FIG. 47. Isolated tumors were fixed and embedded in wax, followed by H&E staining (S4E, bar=200 μm). Representative images are shown.

FIG. 54 Tumors are isolated on day 28 as described in FIG. 47. Isolated tumors were fixed and embedded in wax, followed by p53 IHC staining by DO1 antibody (S4F, bar=200 μm). Representative images are shown.

FIG. 55 The percentage of mCD45+ and hCD45+ cells in PB on day 16, 22, and 26, as described in FIG. 48.

FIG. 56 Combination of ATO and DNA-damaging agents to cancer cells. H1299 cells expressing tet-off-regulated p53-R175H were treated with indicated chemotherapy agents for 12 hr in absence of ATO (p53-R175H panel) or in presence of ATO (PANDA-R175H panel). Indicated proteins were probed. Low bar graph shows the relative level of probed proteins. CIS: Cisplatin; ETO: Etoposide; ADM: Adriamycin (Doxorubicin).

FIG. 57 Trial of ATO and DNA-damaging agents to treat AML.MDS. 50 AML/MDS were recruited for p53 mutation-based precise trial. The two patients harboring de novo ATO-rescuable mp53 and the one patients harboring therapy-related mp53 were administrated with first-line agent Decitabine in combination of ATO.

FIG. 58 A batch of mp53s with mutations on S241 can be rescued by ATO. H1299 cells were transfected with indicated mp53s and treated with ATO, followed by PAb1620 IP (upper panel) and protein level determination (lower panel).

FIG. 59 Summary of ATO's potential in rescuing mp53 structure and induction of PUMA and p21.

FIG. 60 Left panel, H1299 cells expressing tet-off-regulated p53-R175H were treated with indicated chemotherapy agents for 12 hr in absence of ATO (p53-R175H panel) or in presence of ATO (PANDA-R175H panel). Indicated proteins were probed. In mp53 switch-off panel, cells were pretreated with Dox for 48 hr to delete p53-R175H. Low bar graph shows the relative level of probed proteins. CIS: Cisplatin; ETO: Etoposide; ADM: Adriamycin (Doxorubicin); 5-FU: 5-Fluorouracil; ARA: Cytarabine; AZA: Azacitidine; DAC: Decitabine; TAX: Paditaxel. Right panel, cell lysate as above was pretreated with CIP to dephosphorylate cellular proteins. Indicated proteins were probed.

FIG. 61 H1299 cells were transfected with indicated mp53s and treated with ATO, followed by PAb1620 IP (upper panel) and protein level determination (lower panel).

FIG. 62 H1299 cells were transfected with indicated mp53s and treated with ATO, followed by PAb1620 IP.

FIG. 63 Cartoon comparing known computer modelled previously reported compounds versus PANDA Agent described in this application. Left panel, Some of the previously reported compounds were in silico predicted to bind C124, a residue locating on the PANDA Pocket. However, these compounds fail to rescue mp53 efficiently. The binding between these compounds and C124 need to be experimentally confirmed. Middle panel, in our co-crystal of PANDA, we discovered As atom binds PANDA Triad tightly and stabilizes mp53 and thereafter rescues mp53 efficiently. In case of 5-valance arsenic, the R1 and R2 can locate outside of PANDA Triad. Right panel, in current application, PANDA Agent tightly binds one or more residues from PANDA Pocket and stabilizes mp53 and thereafter rescues mp53 efficiently.

FIG. 64 Exemplary reaction for PANDA Agent. A compound containing X group with the capacity to bind a first cysteine (C₁) and/or a second cysteine (C₂) and/or a third cysteine (C₃) binds to one or more PANDA Cysteines. Examples of C₁, C₂, and C₃ includes 0, S, Cl, F, I, Br, OH, and H. C₁, C₂, and/or C₃ can bind to each other. X group includes for example a metal, such as an bismuth, a metalloid, such as an arsenic and an antimony, a group such as a Michael acceptor and/or a thiol, and/or any analogue with cysteine-binding ability. The PANDA Agent can undergo a hydrolysis before reacting and binding to p53 forming PANDA. In some cases, when a group cannot undergo hydrolysis, and accordingly cannot bind to a cysteine. In such cases, the remaining group(s) with cysteine binding potential binds to p53. R₁ and R₂ represent any groups bound to X. R₁ and/or R₂ can also be empty.

FIG. 65 Exemplary reaction for a PANDA Agent with tri-cysteine binding potential. 3-valence ATO undergoes hydrolysis, covalently binds to three PANDA Cysteines on p53.

FIG. 66 Exemplary reaction for a PANDA Agent with tri-cysteine binding potential. 5-valence As compound undergoes hydrolysis, covalently binds to three PANDA Cysteines on p53.

FIG. 67 Exemplary reaction for a PANDA Agent with bi-cysteine binding potential. The PANDA Agent can bind to PANDA Cysteines, or to PANDA Cysteines (Cys₁₂₄, Cys₁₃₅, or Cys₁₄₁), or Cys₂₇s and Cys₂₇₇ or C₂₃e and C₂₄₂.

FIG. 68: Exemplary reaction for a PANDA Agent with mono-cysteine binding potential. The PANDA Agent can bind to PANDA Cysteines, (i.e. Cys₁₂₄, Cys₁₃₅, or Cys₁₄₁) or the other 3 cysteines on PANDA Pocket (Cys₂₃₈, Cys₂₇₅, or Cys₂₇₇).

FIG. 69: Selected Human TP53 Isoforms.

FIG. 70: ATO greatly increases mp53 stability by increasing its melting temperature. Panel A shows the melting curve of the purified p53 core domain R175H(94-293) (“p53C”) recorded via differential scanning fluorimetry at the indicated ratio of ATO in pH 7.5 HEPES buffer. Panel B shows ATO and the purified recombinant p53C (p53C-WT, p53C-R175H, p53C-G245S, p53C-R249S and p53C-R282W, 5 μM for each reaction) were mixed at the indicated ratios in pH 7.5 HEPES buffer for overnight. Melting curves of the p53C were measured by DSF in pH 7.5 HEPES buffer. The apparent Tₘ of the p53C-R175H, p53C-G245S, p53C-R249S, and p53C-R282W can be raised by 1.1-6.5C by maximum in pH 7.5 HEPES buffer. The melting temperatures of p53 core were shown (mean±SD, n=3). Panel C shows melting curve of the purified p53 core domain R175H(94-293) recorded via differential scanning fluorimetry at the indicated ratio of ATO in pH 7.5 HEPES, 150 mM NaCl buffer. Panel D shows ATO and the purified recombinant p53C (p53C-WT, p53C-R175H, p53C-G245S, p53C-R249S and p53C-R282W, 5 μM for each reaction) were mixed at the indicated ratios in pH 7.5 HEPES, 150 mM NaCl buffer for overnight. Melting curves of the p53C were measured by DSF in pH 7.5 HEPES, 150 mM NaCl buffer. The apparent Tₘ of the p53C-R175H, p53C-G245S, p53C-R249S, and p53C-R282W can be raised by 1.0-5.1° C. by maximum in pH 7.5 HEPES, 150 mM NaCl buffer. The melting temperatures of p53 core were shown (mean±SD, n=3). Panel E shows melting curve of the purified p53 core domain (p53C-WT, p53C-G245S, p53C-R249S and p53C-R282W) were recorded via differential scanning fluorimetry at the indicated ratio of ATO in pH 7.5 HEPES buffer. Panel F shows melting curve of the purified p53 core domain (p53C-WT, p53C-G245S, p53C-R249S and p53C-R282W) were recorded via differential scanning fluorimetry at the indicated ratio of ATO in pH 7.5 HEPES, 150 mM NaCl buffer.

FIG. 71: PANDA regains transcriptional activities on most of the p53 target genes. SaOS-2 cells transfected with wtp53, p53-R273H or p53-R282W were treated with 1 μg/ml ATO for 24 hr. Expression levels of the p53 targets were determined by RNA-sequencing. Panel A shows the heatmap of the fold change values (the indicated sample groups versus vector) of a set of 116 reported p53-activated targets. Panel B shows the heatmap of the fold change values of a set of 127 reported p53 targets. Grey scale represents fold change. “vec” means vector.

### 5. DETAILED DESCRIPTION

### 5.1 Interpretations and Definitions

Unless otherwise indicated, this description employs conventional chemical, biochemical, molecular biology, genetics and pharmacology methods and terms that have their ordinary meaning to persons of skill in this field. All publications, references, patents and patent applications cited herein are hereby incorporated herein by reference in their entireties.

As used herein, the biological sample corresponds to any sample taken from a subject, and can include tissue samples and fluid samples such as blood, lymph or interstitial fluid and combinations thereof and the like.

As used in this specification and the appended claims, the following general rules apply. Singular forms “a,” “an” and “the” include plural references unless the content dearly indicates otherwise. General nomenclature rules for genes and proteins also apply. That is, genes are italicized or underlined (e.g.: TP53 or TP53), but gene products, such as proteins and peptides, are in standard font, not italicized or underlined (e.g.: p53). General rules for nomenclature of amino acid location also applies; that is, the amino acid abbreviation followed by number (e.g.: R175, R 175, R-175), where the amino acid name is represented by the abbreviation (e.g.: arginine by “R,” “arg,” “Arg” any other abbreviations familiar to those skilled in the art) and the location of the amino acid on the protein or peptide is represented by the number (e.g.: 175 for position 175). General rules for nomenclature of mutations also apply; for example, R175H, means arginine at location 175 is substituted by histidine. As another example mutation on p53 at location 175 from R to H can be represented by for example “p53-R175H” or “mp53-R175H.” Unless specified otherwise, any amino acid position corresponds to the amino acid location on a wildtype p53, preferably the human wtp53 isoform “a” listed in Section 7.24. General nomenclature rules for organism classification also apply. That is order, family, genus and species names are italicized.

As used herein, the following terms shall have the specified meaning. The term “about” takes on its plain and ordinary meaning of “approximately” as a person of skill in the art would understand, and generally plus or minus 20%, unless specified otherwise. The term “comprise,” “comprising,” “contain,” “containing,” “include,” “including,” “include but not limited to,” or “characterized by” is inclusive or open-ended and does not exclude additional, unrecited elements.

As used herein, the following terms shall have the specified meaning:

“diagnosis” means any method to identify a particular disease, and includes, among others, detecting the symptoms of a disease, assessing the severity of the disease, determining the stages of the disease, and monitoring the progression of the disease.

“expression” or “level of expression” means the level of mRNAs or proteins encoded by the gene marker.

“prognosis” means any method to determine the likely course of a disease, and includes, among others, determining the predisposition of a disease, determining the likelihood a disease will onset, assessing the likely severity of the disease, determining the likely stages of the disease, and predicting the likely progression of the disease.

“screening of effective treatments” means screening of effective therapeutic product or method for the treatment of a certain disease. It can involve in vitro and/or ex vivo screening methods, and includes, among others, both the product or composition to treat a disease and the method to prepare the composition for treatment.

“subject” means any organism. It includes animal, including vertebrate, further including a mammal such as a human. It also includes any unborn child and any un-conceived, hypothetical child of two parents.

“treatment” means the administration and/or application of therapeutic product or method to a subject with a certain disease, and includes, among others, monitoring the efficacy of a type of treatment for the disease.

“PANDA” means a complex comprised of one or more p53 and one or more PANDA Agent.

“PANDA Agent” means a composition of matter capable of binding to the PANDA Pocket that has one or more useful characteristics, examples of such useful characteristics include: (a) can cause a substantial increase in the population of properly folded p53, preferably the increase is at least about 3 times more than the increase caused by PRIMA-1, more preferably the increase is at least about 5 times more than the increase caused by PRIMA-1, further preferably the increase is at least about 10 times more than the increase caused by PRIMA-1, further preferably the increase is at least about 100 times more than the increase caused by PRIMA-1; (b) can cause a substantial improvement in the transcriptional function of p53, preferably the improvement is at least about 3 times more than the improvement caused by PRIMA-1; more preferably the improvement is at least about 5 times more than the improvement caused by PRIMA-1, further preferably the improvement is at least about 10 times more than the improvement caused by PRIMA-1, further preferably the improvement is at least about 100 times than the improvement caused by PRIMA-1; and (c) can cause a substantial enhancement of stabilization of p53 as measured by, for example, an increase p53 Tm, preferably the enhancement is at least about 3 times more than the enhancement caused by PRIMA-1, more preferably the improvement is at least about 5 times more than the improvement caused by PRIMA-1, further preferably the improvement is at least about 10 times more than the improvement caused by PRIMA-1, further preferably the improvement is at least about 100 times than the improvement caused by PRIMA-1. A PANDA Agent is preferably to have two or more useful characteristics and more preferably has three or more useful characteristics. Exemplar PANDA Agents is ATO and its analogs. More exemplar PANDA Agents can be found in Table 1-Table 7.

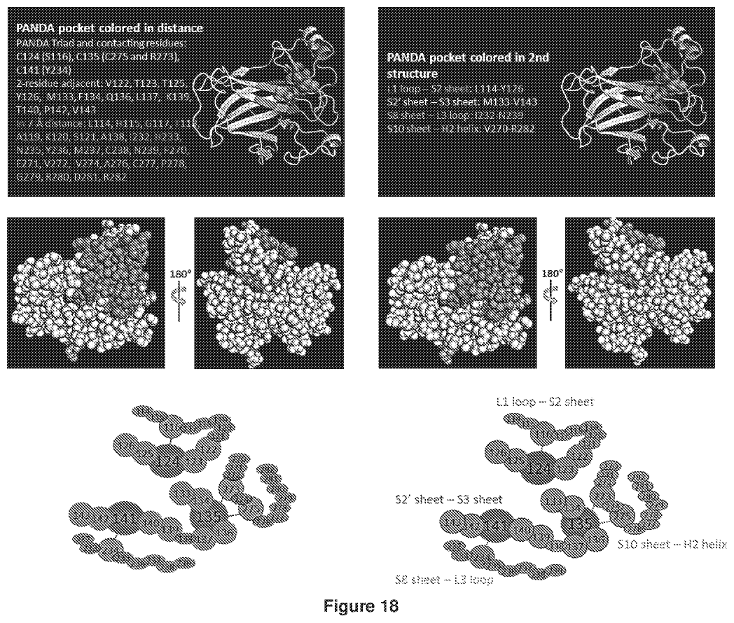

“PANDA Pocket” means a region consisting essentially of an area of about 7 Å from a properly folded PANDA Cysteine, including, all amino acids adjacent to one or more properly folded PANDA Cysteine, all amino acids that contact with one or more properly folded PANDA Cysteine, and all PANDA Cysteines. Exemplar 3D structures of a PANDA Pockets can be found FIG. 14, FIG. 18, Appendix A and Appendix B. In an exemplary embodiment, the PANDA Pocket can include all of the above amino adds, a subset of the above amino acids, and possibly other components as long as the resulting tertiary structure comprising the PANDA Pocket exhibits one or more of the useful characteristics described in this application. Thus, the PANDA Pocket can comprise or consist essentially of the above amino acids, or a subset thereof.

“PANDA Core” means the tertiary structure formed on the PANDA Pocket of a p53 when a PANDA Agent forms at least one tight association between the PANDA Pocket and the PANDA Agent.

“PANDA Cysteine” means a cysteine corresponding to the wtp53 positions cysteine 124 (“C124” or “cys124”), cysteine 135 (“C135” or “cys135”), and cysteine 141 (“C141” or “cys141”) (together the “PANDA Triad”).

“p53” means any wildtype p53 (“wtp53”), including all natural and artificial p53; any mutated p53 (“mp53”), including all natural and artificial p53; or a combination thereof.

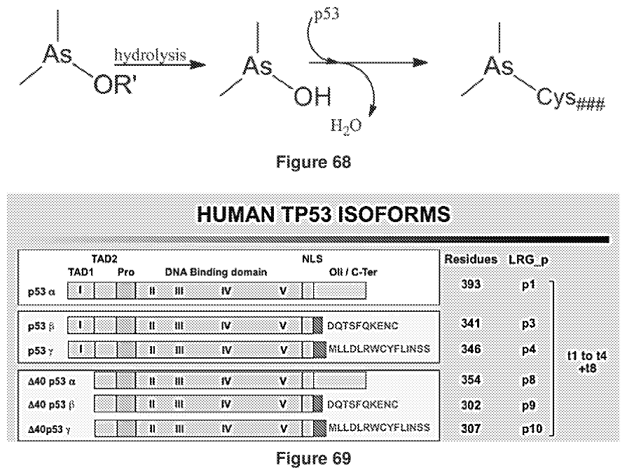

“wtp53” means all wildtype p53 that is commonly considered as wildtype, or has a wildtype sequence, and includes any commonly acceptable variations, such as variations caused by single nucleotide polymorphism (“SNP”). Exemplar wtp53 can be found in FIG. 64-FIG. 68.

“SNP” means single-nucleotide polymorphism, which is a variation in a single nucleotide that occurs at a specific position in the genome, where each variation is presented to some appreciable degree within a population. An exemplary list of known SNP on p53 is Table 8.

“mp53” means mutated p53, which includes all p53 and p53 like macromolecules that is not a wtp53. mp53 includes, artificial mp53, such as recombinant p53, chimeric p53, p53 derivative, fusion p53, p53 fragment, and p53 peptide. Exemplar mp53 include one or more mutations corresponding to the wtp53 positions R175, G245, R248, R249, R273, R282, C176, H179, Y220, P278, V143, I232, and F270. Exemplar mp53 mutations include R175H, G245D/S, R248QW, R249S, R273C/H, R282W, C176F, H179R, Y220C, P278S, V143A, I232T, and F270C mutations.

“mp53 hotspot” means a mutation on mp53 located at R175, G245, R248, R249, R273, or R282.

“hotspot mp53” means an mp53 with at least one mutation in mp53 hotspots, namely, R175, G245, R248, R249, R273, R282, and combinations thereof.

“biological system” means a cell, bacteria, artificial system containing p53 pathway and relevant proteins.

“p53 inhibiting protein” means a protein that inhibits a function of activity of p53, and includes, for example, murine double minute 2 (“MDM2”), inhibitor of apoptosis-stimulating protein of p53 (“iASPP”) and sirtuin-1 (“SIRT1”).

“Contacting mp53” means a mp53 that loses its DNA binding ability without drastically affecting the p53 structure. Contacting mp53s are represented by, for example, p53-R273H, p53-R273C, p53-R248Q and p53-R248W.

“Structural mp53” means a mp53 that has significantly disrupted three-dimensional structure as compared to wtp53. Structural mp53s are represented by, for example, p53-R175H, p53-G245D, p53-G245S, p53-R249S, and p53-R282W.

“useful characteristics” a means capable of efficiently and effectively rescuing at least one of mp53 structure, transcriptional activity, cell growth inhibition, tumor-suppressive function to that of wtp53. Exemplar useful characteristics include: (a) can cause a substantial increase in the population of properly folded p53, preferably the increase is at least about 3 times more than the increase caused by PRIMA-1, more preferably the increase is at least about 5 times more than the increase caused by PRIMA-1, further preferably the increase is at least about 10 times more than the increase caused by PRIMA-1, further preferably the increase is at least about 100 times more than the increase caused by PRIMA-1; (b) can cause a substantial improvement in the transcription function of p53, preferably the improvement is at least about 3 times more than the improvement caused by PRIMA-1; more preferably the improvement is at least about 5 times more than the improvement caused by PRIMA-1, further preferably the improvement is at least about 10 times more than the improvement caused by PRIMA-1, further preferably the improvement is at least about 100 times than the improvement caused by PRIMA-1; and (c) can cause a substantial enhancement of stabilization of p53 as measured by, for example, an increase p53 Tm, preferably the enhancement is at least about 3 times more than the enhancement caused by PRIMA-1, more preferably the improvement is at least about 5 times more than the improvement caused by PRIMA-1, further preferably the improvement is at least about 10 times more than the improvement caused by PRIMA-1, further preferably the improvement is at least about 100 times than the improvement caused by PRIMA-1. A PANDA Agent is preferably to have two or more useful characteristics and more preferably has three or more useful characteristics. Exemplar PANDA Agents is ATO and its analogs. More exemplar PANDA Agents can be found in Table 1-Table 7

“DTP” means Developmental Therapeutics Program as understood by a person of ordinary skill in the art.

“ATO” or “As₂A” means arsenic trioxide and compounds generally understood as arsenic trioxide.

“analog” or “analogue” means a compound obtained by varying the chemical structure of an original compound, for example, via a simple reaction or the substitution of an atom, moiety, or functional group of the original compound. Such analog may involve the insertion, deletion, or substitution of one or more atoms, moieties, or functional groups without fundamentally altering the essential scaffold of the original compound. Examples of such atoms, moieties, or functional groups include, but are not limited to, methyl, ethyl, propyl, butyl, hydroxyl, ester, ether, acyl, alkyl, carboxyl, halide, ketyl, carbonyl, aldehyde, alkenyl, azide, benzyl, fluoro, formyl, amide, imide, phenyl, nitrile, methoxy, phosphate, phosphodiester, vinyl, thiol, sulfide, or sulfoxide atoms, moieties, or functional groups. Many methods for creating a chemical analog from an original compound are known in the art.

“a therapeutically effective amount” is an amount of a compound effective to prevent, alleviate, or ameliorate symptoms of a disorder or prolong the survival of the subject being treated. Determination of a therapeutically effective amount is well within the capability of those skilled in the art, especially in light of the detailed disclosure provided herein. The effective dosage, level, or amount of a compound to be used in vivo can be determined by those skilled in the art, taking into account the disorder to be treated, the condition of the individual patient, the site of delivery, the method of administration, the potency, bioavailability, and metabolic characteristics of the compound, and other factors.

“efficiently” as used to describe enhancement for a useful characteristics, such as rescuing one or more wtp53 structure or function, rescuing one or more wtp53 transcriptional activity, cell growth inhibition activity, tumor-suppressive function to that of wtp53, generally means enhancing the useful characteristics by more than 3 times, as compared to the enhancement by PRIMA-1, preferably 5 times, more preferably 10 times, more preferably 100 times. For example, an efficient enhancement would be enhancing the Tₘ of mp53 by 3-100 times of those of PRIMA-1, and/or folds mp53 by 3-100 times of those of PRIMA-1, and/or stimulates mp53's transcriptional activity by 3-100 times of those of PRIMA-1.

Examples of a p53 related disorder include cancer, such as lung, breast, colorectal, ovarian, and pancreatic cancers; a tumor, a consequence of aging, a developmental disease, accelerated aging, an immunological disease.

### 5.2 p53 is One of the Most Important Proteins in Cell Biology

p53 is one of the most important proteins in cell biology. The apparently 53-kilodalton protein p53 is a transcription factor. Wildtype p53 (wtp53) has a sequence that has been identified. (See public gene banks, such as gene bank, protein bank, Uniport; see also Section 7.25). Exemplar wtp53 sequences are listed under Section 7.25). Unless specified otherwise, this application uses the wtp53 sequences of human p53 isoform “a” listed under Section 7.25 to reference locations.

The human wtp53 is active as a homotetramer of 4×393 amino acids with multiple domains including an intrinsically disordered N-terminal transactivation domain (“TAD”), a proline-rich domain (“PRD”), a structured DNA-binding domain (“DBD”) and tetramerization domain (“TET”) connected via a flexible linker, and an intrinsically disordered C-terminal regulatory domain (“CTD”). Many p53 family genes expressing multiple isoforms exist, and often exhibit antagonistic functions.

Wtp53 plays a central part in the cells and is frequently considered as the most important tumor suppressor. Upon cellular stresses, such as DNA damage or oncogenic stress, p53 is activated and transcriptionally regulates a batch of genes (for example, Apaf1, Bax, Fas, Dr5, mir-34, Noxa, TP53AIP1, Perp, Pidd, Pig3, Puma, Siva, YWHAZ, Btg2, Cdkn1a, Gadd45a, mir-34a, mir-34b/34c, Prl3, Ptprv, Reprimo, Pai1, Pml, Ddb2, Ercc5, Fancc, Gadd45a, Ku86, Mgmt, Mih1, Msh2, P53r2, Polk, Xpc, Adora2b, Aldh4, Gamt, Gls2, Gpx1, Lpin1, Parkin, Prkab1, Prkab2, Pten, Sco1, Sesn1, Sesn2, Tigar, Tp53inp1, Tsc2, Atg10, Atg2b, Atg4a, Atg4c, Atg7, Ctsd, Ddit4, Dram1, Foxo3, Laptm4a, Lkb1, Pik3r3, Prkag2, Puma, Tpp1, Tsc2, Ulk1, Ulk2, Uvrag, Vamp4, Vmp1, Bai1, Cx3cl1, Icam1, Irf5, rf9, Isg15, Maspin, Mcp1, Ncf2, Pai1, Tlr1-Tlr10, Tsp1, Ulbp1, Ulbp2, mir-34a, mir-200c, mir-145, mir-34a, mir-34b/34c, and Notch1) to trigger cell-cycle arrest, DNA repair, apoptosis, cell repair, cell death and others. Apart from anti-cancer role, p53 target genes also have important roles in senescence, angiogenesis, and autophagy, connecting, regulating oxidative stress, regulating metabolic homeostasis, stem cell maintenance, and others.

A mutation to wtp53 can have a wide range of implications. The p53 protein is such a powerful tumor suppressor that it is inactivated by mutation in nearly half of all human cancers. A mutation to wtp53 can have a wide range of implications. First, the resultant p53 protein, mutant p53 (“mp53”), will substantially lose its tumor-suppressive function. mp53 expressing mice and humans develop a large number of cancer types at early onset. Second, some of the mp53s will, in addition gain oncogenic properties, such as, for example, promoting cancer metastasis, conferring resistance to treatment, and causing cancer patients to relapse.

Accordingly, understanding p53, and more importantly, achieving structural and functional restoration of mp53, is the holy grail of modern cell biology, medicine, and cancer research. p53 is the most actively researched protein in cancer, medicine, and biology. Moreover, research in p53 far exceeds that being done with respect to even the second most actively researched protein, namely, TNF, by 60%, and exceeds the third most actively researched protein, namely, EGFR, by 80% (Dolgin, 2017). Since 2001, p53 has been on the top of the most actively researched proteins, far exceeding others. One of the reason for this is that p53 is the most commonly mutated protein in cancer, far exceeding other cancer mutations (Kandoth et al., 2013).

### 5.3 p53 and Cancer

Around half of all human tumors harbor partially functional, but silent wtp53s, while the other half carry mutant p53s (Vogelstein et al., 2000). Mouse studies suggest that restoration of wtp53 function can completely or partially regress tumor growth (Feldser et al., 2010; Martins et al., 2006; Ventura et al., 2007; Xue et al., 2007).

Most existing efforts toward restoring wtp53 function have focused on p53 inhibiting proteins (“PIP”), including murine double minute 2 (“MDM2”), inhibitor of apoptosis-stimulating protein of p53 (“iASPP”), and sirtuin-1 (“SIRT1”). For example, since amplification of MDM2 or loss of p14^{ARF}, its inhibiting protein, closely correlates with sarcomas and glioblastomas, respectively (see TCGA database) (Gao et al.), the MDM2-inhibiting compound, Nutin, was identified to counteract MDM2's activities (Vassilev et al., 2004). As another example, we have reported that iASPP exposes the RaDAR nudear localization code (Lu et al., 2014), enters the nudeus (Lu et al., 2016a), and inhibits wtp53 in metastatic melanoma (Lu et al., 2013), and accordingly, we are exploring ASPP inhibiting compounds. Many of these anti-PIP compounds are highly efficient, have a clear mechanism of action (“MOA”), and are progressing to clinical investigations (Khoo et al., 2014).

Others are attempting to target mp53, a protein that not only loses its tumor suppressive function but also frequently gains oncogenic properties. This approach, while attractive, is not easy, however.

Simply introducing wtp53 to mp53-expressing cells is problematic because mp53 is dominant-negative, and as seen in a mouse model-based study, can dampen the effect of the exogenous wtp53 introduced (Wang et al., 2011).

Selectively inhibiting mp53-expressing cells by blocking mp53 upstream pathways, downstream pathways, or relevant pathways are also problematic. While the mp53 upstream inhibitor, suberanilohydroxamic acid (“SAHA”), can inhibit the mp53-upstream histone deacetylases (“HDAC”), thereby promoting mp53 degradation (Li et al., 2011); the mp53 downstream inhibitor, statins (a cholesterol inhibitor), can block the mp53-downstream mevalonate pathway, thereby decreasing the survival rate of mp53-expressing cells (Parrales et al., 2016); and certain kinase inhibitors can selectively inhibit mp53-expressing cells by interfering with mp53-associated activation of receptor tyrosine kinase signaling, thereby inhibiting cell invasion by blocking integrin recycling (Muller et al., 2009) all show promises, none of these strategies can restore mp53's tumor-suppressive functions. Accordingly, it is not known whether these strategies are sufficient to treat cancer clinically in the long-term (Muller and Vousden, 2014). Indeed, mouse studies show that eliminating mp53 only extends the survival of mp53-expressing animals to p53^{-/-} (null) levels (Alexandrova et al., 2015).

### 5.4 Promises and Challenges of Rescuing Mp53

The tumor-suppressive functions of mp53 were reported to be rescuable in 1993 (Halazonetis and Kandil, 1993; Hupp et al., 1993). Since then, identifying an efficient, effective, mp53 specific rescue agent has been the holy grail of cancer biology and medicine. Indeed the direct medical expenses for mp53 patients in 2017 alone amounts to approximately 65 billion USD. By successfully identifying a highly efficient and effective mp53 rescue agent, Applicants seek to address the tremendous financial, physical and emotional hardships faced by these mp53 patients and their families.

Despite countless screening efforts of varying scale, there is still no efficient and effective mp53 rescue agent. This is partly because rescuing mp53's tumor suppressive function (Joerger and Fersht, 2016; Muller and Vousden, 2013, 2014) is extremely challenging (Joerger and Fersht, 2016; Muller and Vousden, 2013, 2014) (Bykov et al., 2017) (Sabapathy and Lane, 2018). In fact, it may be one of the most difficult scientific problems of our generation. To successfully rescue mp53, the rescue agent must do more than simply inhibit or destroy specific mp53 functions. The rescue agent must repair or rescue the wildtype functions of mp53.

Without a doubt, rescue is much more challenging than destruction. Understandably, of the over 80 clinically approved targeted drugs, the vast majority of them are inhibitors of oncoproteins. None of them can rescue a tumor suppressor's function.

To add to the challenge, like RAS, the mp53 surface provides no obvious druggable pocket (Joerger and Fersht, 2016). Accordingly, despite having more than 15 mp53 rescue candidates reported in the past two decades and having attracted tens, and even hundreds, of millions of dollars in investments, to date, only one candidate (PRIMA-1/APR-246) has entered a clinical trial. Even among the 15 reported mp53 rescue candidates, all of them have barely detectable efficacy, with an increase of less than 2 times for structural rescue, and with an increase of less than 2 times for transcriptional rescue. By comparison, a fully rescued p53-R175H is about 100 times for structural rescue. As another example, a fully rescued p53-282 20× for functional fully rescue. Moreover, the MOA for these rescue agents are largely unknown (Bykov et al., 2017) (Sabapathy and Lane, 2018). With these unfavorable numbers and without any clear MOA, the utility of these rescue candidates for cancer therapy is very limited.

Accordingly, Applicants have made it their priority to identify a highly efficient and effective rescue agent that directly rescue mp53 with a clear MOA.

5.4.1 mp53 Rescue Agents Identified by In Vitro Screenings are not Ideal

Initial screenings for mp53 rescue agents were primarily based on mp53 recombinant proteins in vitro. These include CP-31398, which was identified because it promoted recombinant mp53 stability (Foster et al., 1999) and SCH529074 and p53R3, which were identified because they improved recombinant mp53 to DNA binding (Demma et al., 2010; Weinmann et al., 2008). However, these rescue agents are inefficient, nonspecific, and face serious challenges in cells.

For example, CP-31398 was shown to have limited efficiency in cells. Not only do they have limited specificity to mp53 and can cause substantial toxicity to the cells, they may also have trouble entering the cells. Moreover, it is reported that the toxic effect is nonspecific to and independent of mp53 expression (Rippin et al., 2002), suggesting that CP-31398 does not function by directly targeting mp53. Furthermore, unlike earlier in vitro studies, which show CP-31398 binds to the mp53 protein, later in vivo studies show that CP-31398 binds to DNA in cells instead (Rippin et al., 2002).

5.4.2 mp53 Rescue Agents Identified by Cell-Based Screenings are not Ideal

In 2002, a cell-based screening found PRIMA-1 and MIRA to selectively inhibit mp53 expressing cells (Bykov et al., 2002). In silico screenings found NSC319726 to selectively inhibit a panel of mp53 expressing cell lines (Yu et al., 2012). Another cell-based screening found Chetomin to enhance mp53-dependent luciferase reporter activity in cells (Hiraki et al., 2015). However, like the in vitro screenings, the rescue agents identified by cell-based screenings are also problematic.

Using PRIMA-1 as an example, studies have shown that, like the other rescue agents, it has limited rescue efficiency. Moreover, an increasing number of studies have reported that PRIMA-1 and its structural analog PRIMA-1Met (“APR-246”) inhibited cell growth irrespective of whether mp53 is present or not (Aryee et al., 2013; Grellety et al., 2015; Lu et al., 2016b; Patyka et al., 2016; Tessoulin et al., 2014). In addition, studies have increasingly reported that PRIMA-1 targets oxidative stress signaling components (Bauer et al., 2016; Joerger and Fersht, 2016; Lambert et al., 2009) and that the observed sensitivity caused by PRIMA-1 and other alkylating agents, such as PK11007, to mp53 expressing cells is contributed by a loss of antioxidant functions in mp53s (Bauer et al., 2016; Joerger and Fersht, 2016; Lambert et al., 2009)

Furthermore, studies have increasingly reported confusing results and questioned their MOA and pointed to their limited efficiency. In addition, while Lambert and Bykov reported that PRIMA-1 binds to mp53 covalently and promotes mp53-DNA binding activity in vitro (Bykov et al., 2002; Lambert et al., 2009), at least one other study reported that it is CP-31398, but not PRIMA-1, that restores DNA-binding activity to mp53 in vitro (Demma et al., 2004). These findings appear to be at odds with the initial reports that PRIMA-1 selectively inhibit p53-R273H-expressing Saos-2 cells (Bykov et al., 2002). In fact, these later studies suggest that PRIMA-1 does not directly target and rescue mp53 and may instead be killing mp53 cells by synthetic lethality, that is, inhibiting other cellular the proteins such as above mentioned oxidative stress signaling components, rather than mp53, that are essential for the survival of mp53 cells. (Weidle et al., 2011). Supporting this theory, studies have shown that many proteins, including CHK1, WEE-1, PLK-1, and ATM, are synthetic lethal targets of mp53 cells (Weidle et al., 2011). In one clinical trial, WEE-1 inhibitor had efficacy in treating patients expressing mp53 (Leijen et al., 2016a; Leijen et al., 2016b).

5.4.3 Having an Established MOA is Crucial for Identifying an Effective and Efficient Rescue Agent for Mp53

Presently, the vast majority of known mp53-rescue agents, including CP-31398 (Foster et al., 1999) and PRIMA-1 (Bykov et al., 2002), are thought to stabilize the p53's wild-type structure (Muller and Vousden, 2014). However as discussed above, they are not ideal. One of the major problems with these rescue agents is that their MOA unclear.

The vast majority of reported rescue agents were identified via random screenings. Only very few, such as PhiKan083 and PK7088 (Basse et al., 2010; Boeckler et al., 2008; Liu et al., 2013), were identified via rational screenings. However, these compounds can only rescue p53 with mutation on Y220. Accordingly, the MOA for the vast majority of rescue agents remains largely unknown.

This is particularly problematic. As seen for example in PRIMA-1, not knowing the MOA of an rescue agent or which proteins it targets in cells (Joerger and Fersht, 2016) can lead to puzzling results and conflicting theories. To further illustrate, without a concrete MOA, it is puzzling why many of the identified rescue agents can rescue different categories of mp53s.

In general, the vast majority of wtp53 populations are properly folded and thus, functional. When p53 mutates, it falls roughly into two categories: (1) contacting mp53 that loses its DNA binding ability without drastically affecting the p53 structure (“Contacting mp53”); and (2) structural mp53 that has disrupted three-dimensional structures as compared to the wildtype (“Structural mp53”). A representative Contacting mp53 is p53-R273H, with other common examples, including p53-R273C, p53-R248Q and p53-R248W. A representative Structural mp53 is p53-R175H, with other common examples, including mp53s include p53-G245D, p53-G245S, p53-R249S, and p53-R282W. Accordingly, for Structural mp53s, the population of unfolded p53s dramatically increase. To rescue Structural mp53s, one would need to increase the population of unfolded p53s to folded p53s.

Because these mp53s lose their wildtype p53 function indifferent ways, it would be reasonable that a rescue agent for one category of mp53 would not rescue the other category. In fact, there is a proposition to classify mp53s into five different categories, where each category has its own specific set of rescue requirements (Bullock and Fersht, 2001; Bullock et al., 2000; Joerger and Fersht, 2007).

In general, it is substantially more challenging to rescue Contacting mp53 than Structural mp53. For example, to compensate for Contacting mp53's loss of DNA-contact residue(s), such as R248 and R273, the rescue agent must create a new contact for DNA binding (Joerger and Fersht, 2007). Accordingly, without a defined MOA, it is puzzling how a single rescue agent, such as CP-31398 (Foster et al., 1999), PRIMA-1 (Bykov et al., 2002), SCH529074 (Demma et al., 2010), Zinc (Puca et al., 2011), stictic acid (Wassman et al., 2013), and p53R3 (Weinmann et al., 2008), can rescue both Structural mp53s (such as p53-R175H) and Contacting mp53s (such as p53-R273H) (Joerger and Fersht, 2016; Khoo et al., 2014).

We believe one of the most important deficiencies of existing screening methods, both protein-based and cell-based, is that their selection criteria are more or less random. Accordingly, they fail to elucidate the MOA of the rescue agents they identify. Here, Applicants have set out to develop a novel method for a rational, effective screening of mp53 rescue candidates with and a concrete MOA.

### 5.5 4C Screening—a Method for Rational, Effective Screening of Mp53 Rescue Candidates

The first rationally designed screening was carried out in 2008, in which mp53s were differentially treated and structurally analyzed (Basse et al., 2010; Boeckler et al., 2008). Since mp53s are highly diverse, a rational basis was developed to analyze individual mp53s (Joerger and Fersht, 2007; Joerger and Fersht, 2016; Muller and Vousden, 2013; Muller and Vousden, 2014). PhiKan083 and PK7088 were identified through this screening and were found to selectively bind and rescue p53-Y220C with an intelligible MOA. However, p53-Y220C is not among the six most frequently occurring mp53s, there is a need to identify a rescue agent capable of rescuing a broader range of mp53s.

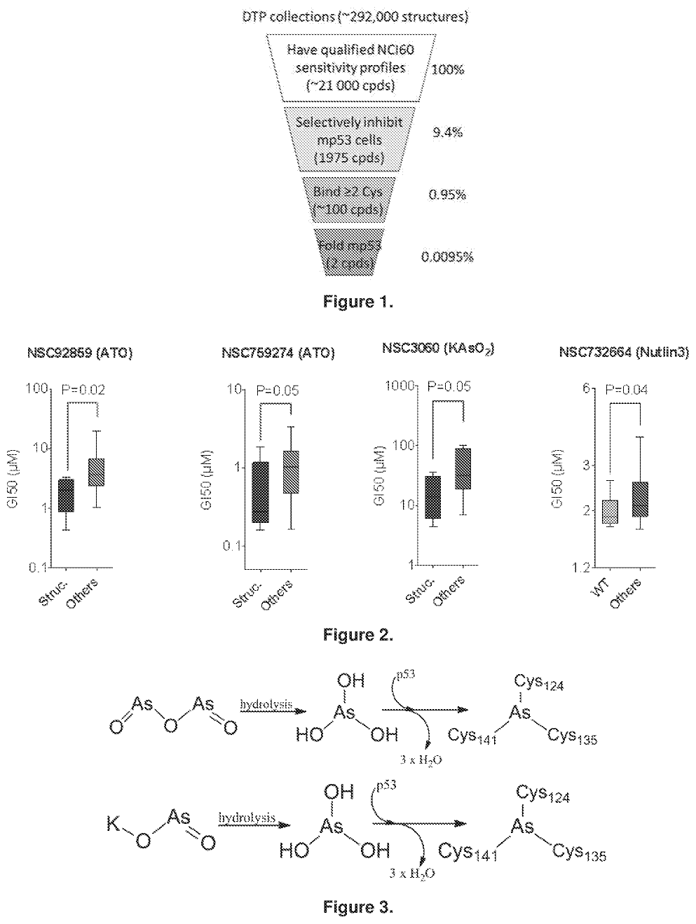

As explained above, there is a need for an efficient, rational based screen to identify rescue agents for hotspot mp53s with desirable characteristics and a concrete MOA. Others have attempted, but this need has not been filled. Here, we disclose such a screening method, an efficient, rational based screening method that integrates in silico rational Classification of mp53s, in silico rational analysis of Compound structure, Cell growth assay, and experimental mp53 Conformation determination (“4C Screening”). Using our 4C Screening method, we screened compound repositories, such as the compound repository of DTP (FIG. 1). Our goal was to identify compounds with multiple cysteines-binding potential that can selectively inhibit Structural mp53-expressing cell lines by promoting proper refolding of mp53.

Through our 4C Screening, we can identify rescue candidates that, upon hydroxylation, can simultaneously bind to three cysteines of mp53s; can refold p53-R175H with a strikingly high efficiency, to a level comparable to that of wtp53 as measured by assays, such as by PAb1620 and PAb246 immunoprecipitation; can rescue transcriptional activity of p53-R282W and p53-G245S to a level comparable to that of wtp53 as measured by luciferase report assay; can selectively inhibit mp53 expressing cell lines, such as the NCI60 cell lines that expresses the Structural hotspot mp53; can inhibit mouse xenografts dependent on structural mp53s; and can be used to treat mp53 harboring cancer patients in combination with DNA-damaging agents.

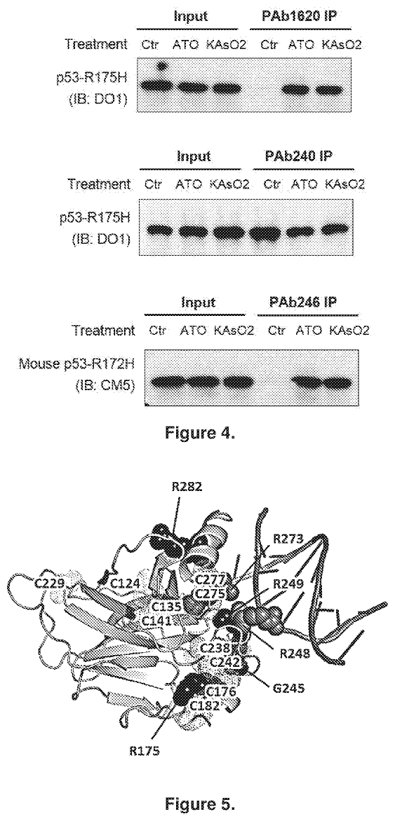

As an example, we predicted ATO (As₂O₃) and NSC3060 (KAsO₂) to be able to simultaneously bind 3 cysteines upon hydroxylation (FIG. 3) and found both ATO and NSC3060 to selectively inhibit structural hotspot mp53s expressing NCI60 cell lines (FIG. 2). Interestingly none of the reported compounds tested, including PRIMA-1 and NSC319726 survived the 4C screening (FIG. 9) even though in the same assays, Nutlin3, as we had expected, was found to selectively inhibit the wtp53 expressing cell lines (FIG. 2). Furthermore, we found that both ATO and NSC3060 refolded p53-R175H with a high efficiency as demonstrated by a measurable increase of PAb1620 epitopes and PAb246 (for mouse p53-R172H) epitopes and a measurable decrease of the PAb240 epitope (FIG. 4). We run comparative studies to confirm that the PAb1620 is specific to the wildtype p53 epitope, as the PAb1620 efficiently immunoprecipitated folded wtp53, but not the unfolded p53-R175H (data not shown).

5.5.1 in-Silico Rational Classification of mp53s

One of the challenges in designing a rational screening method for mp53 rescue agents is that mp53 dysfunctions are diverse. Accordingly, a rational screening strategy designed specifically for different types of mp53 mutation is necessary. In addition, a strategy designed to screen rescue agents that can simultaneously correct the structural defects of Structural mp53s and re-introduce the DNA contacting region of Contacting mp53s may be unrealistic, because such rescue agent may not exist.

There is, however, a class of mp53s that are mainly unfolded at body temperature but refolded (and regains transcriptional activity) at lower temperatures (Bullock et al., 1997; Bullock et al., 2000). For example, the four hotspot Structural mp53s (p53-R175H, p53-G245S/D, p53-R249S, and p53-R282W) (FIG. 5), which are destabilized in varying degrees, belong to this class (Bullock et al., 1997; Bullock et al., 2000). As seen in its representative member, the R282W mutation disrupts the hydrogen-bond network in the local loop-sheet-helix motif, reducing the melting temperature (“Tₘ”) and causing global, structural destabilization.

We thus predicted that a broad spectrum rescue agent, capable of rescuing this class of mp53s, may exist.

5.5.2 Cell Growth Assay

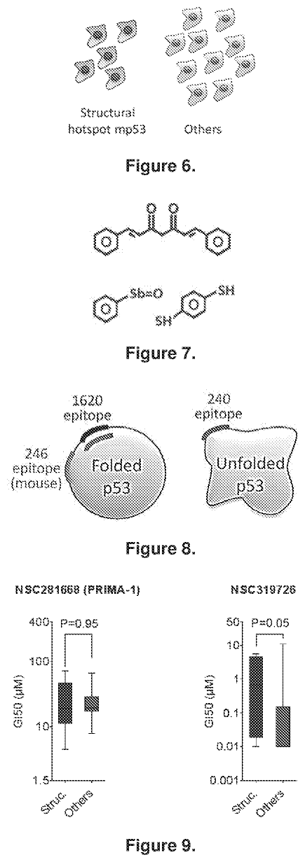

The independently performed NCI60 screening project supplied cell line sensitivity profiles for a large number of the DTP compounds (Shoemaker, 2006). We hypothesized that the compounds that selectively inhibit Structural mp53-expressing NCI60 cell lines would have higher chance to act as stabilizer of this class of mp53s (FIG. 6).

Of the overall approximately 292.000 structures deposited in DTP, approximately 21 000 compounds have sensitivity profiles that passed the quality control according to CellMiner (Reinhold et al., 2012) (FIG. 1). We thus narrowed down the approximately 21 000 compounds by selecting for those that prefer to inhibit cells expressing Structural hotspot mp53s, for example, the class of mp53s we predicted that may be rescued by a broad-spectrum drug (see Section 6.5.1). Using this criteria, we found 1975 compounds to selectively inhibit Structural mp53-expressing NCI60 cell lines with a correlations score >0.33 and p value <0.05 (FIG. 1), lower G150 on structural mp53-expressing lines.

5.5.3 in-Silico Rational Analysis of Compound Structure

Based on our mp53 classification analysis described in Section 6.5.1 above, we predicted that there may be a broad spectrum rescue agent capable of rescuing the class of Structural hotspot mp53s (p53-R175H, p53-G245S/D, p53-R249S, and p53-R282W). In addition, we hypothesize that immobilizing mutation regions may stabilize this class of mp53 globally. Importantly, we found that 8 of the 10 mp53 cysteines are in close proximity to the Structural mp53 hotspots (FIG. 5). Further, we discovered that these cysteines are clustered in pairs, namely as, C176/C182, C238/C242, C135/C141, and C275/C277. Thus, we hypothesized that covalently crosslinking the cysteine pairs and/or dusters can immobilize the local region and thereafter be enough to off-set the flexibility caused by the nearby hotspot mutation(s).

We further narrowed down compounds from the DTP library in-silico, selecting compounds with multiple cysteine-binding potential, such as compounds with heavy metals such as Zn, Hg, As, and Au; thiol containing compounds; and Michael acceptors (FIG. 7). Our 4C Screening method is distinct from, and an improvement over, prior screening methods in many ways. For example, at least conceptually, we selected rescue candidates with multiple cysteine-binding potential, suitable for cysteine crosslinking, instead of selecting rescue candidates with single cysteine-binding potential, suitable for cysteine modification (Kaar et al., 2010; Zache et al., 2008).

After the two-step rational selection process described above, we narrowed an initial pool of 1,975 compounds to a pool of about 100 mp53 rescue candidates.

5.5.4 Experimental Mp53 Conformation Determination

We next experimentally tested whether p53-R175H was properly folded in the presence of the rescue candidates using a wtp53 specific antibody, PAb1620 antibody (Wang et al., 2001), by immunoprecipitation (FIG. 8). The rescue candidates that passed these tests were further confirmed by immunoprecipitation with an antibody specific for folded mouse p53 (PAb246) and an unfolded p53 (PAb240). These conformation analysis ensure the rescue effects observed were directly caused by rescue agents' induced mp53 stabilization, rather than by synthetic lethality. Then, we meticulously tested the ability of the validated mp53 rescue candidates in controlled, experimental settings, to determine their ability to stabilize Structural mp53s, to increase Tₘ, to stimulate transcriptional activity, and to inhibit cancer cells in a mp53 dependent manner.

### 5.6 4C+ Screening

To expand our pool of rescue candidates, we conducted an ultra-large 4C+ Screening. We in silico analyzed approximately 94.2 million compounds derived from PubChem (https://oubchem.ncbi.nlm.nih.ov/). Since we identified two arsenic containing compounds in our 4C Screening, in our 4C+ Screening, we selected compounds containing the metal arsenic or its analogues, such as antimony, and bismuth, with at least one cysteine-binding potential. About 32957 compounds were discovered to contain As, and/or Sb, and/or Bi. Under these criteria, we included any organic five-valence arsenic, five-valence antimony, and five-valence bismuth, as long as they have the potential to bind one or more cysteine. After this in-silico pre-screening step, we in silico narrowed an initial pool of approximately 94.2 million compounds to a pool of thousands of rescue candidates. We then selected and experimentally tested some structural mp53s for their abilities to refold protein, increase Tₘ, and stimulate transcriptional activity.

### 5.7 Identifying Mp53 Rescue Agents by 4C Screening and 4C+ Screening

Nearly half of human cancers harbor a mp53 that loses its tumor-suppressive function and/or frequently gain some oncogenic functions. While dysfunctional p53 mutations are created via a diversity of mechanisms on a variety of sites, approximately one-third of the p53 mutations are located on one of six mp53 hotspots: R175, G245, R248, R249, R273, and R282, (each a “mp53 hotspot”) (Freed-Pastor and Prives, 2012). The resulting mp53s are commonly classified as Contacting mp53 which loses DNA-contacting residue without drastically altering the mp53 structure and Structural mp53 which loses the wtp53 structure. DNA-binding ability and transcriptional activity are greatly impaired in both Contacting mp53s and Structural mp53s. Moreover, most of cancer-derived mp53s lose wtp53's tumor-suppressive functions and many also gain oncogenic properties.

Using our efficient and highly rational 4C Screening, we can experimentally identify at least two wide-spectrum mp53 recusing agents with remarkably high rescue efficiency. They are arsenic trioxide (ATO: NSC92859 & NSC759274) and potassium arsenite (KAsO₂: NSC3060). Our results show that these mp53 rescue agents can rescue mp53's structure; increase thermodynamic stability; rescue mp53's transcriptional activity; rescue mp53's tumor suppressive function in vitro, in vivo, and in patients; rescue different mp53s; remarkable rescue capacity for Structural mp53. We also identified an atom-level rescue mechanism based on these rescue agents.

Using our efficient and highly rational ultra-large-scale 4C+ Screening, we further discovered thousands of clinically relevant and efficient mp53 stabilizers, many of which contain arsenic (Table 1-Table 6). We experimentally confirmed 31 mp53 recusing agents with key supporting data (rescue efficiency on mp53's structure and transcriptional activity) (Table 7). We further disclose here the atom-level mechanism by which Structural hotspot mp53s can be pharmacologically stabilized.

Using our 4C Screen, we discovered that elemental arsenic and its analogues, whether alone or in a compound, rapidly, effectively and selectively stabilizes p53. In particular, we found that elemental arsenic and its analogues are particularly useful for the class of Structural mp53s because they are heavily destabilized. We further discovered that arsenic and its analogues directly and covalently binds mp53s and raises the melting temperature of numerous p53s, particularly the Structural mp53s, including four hotspot Structural mp53s (p53 with mutations on R175, G245, R249, R282), by approximately 1-8° C., supporting that arsenic is covalently bound to the Structural mp53. We further discovered that arsenic and its analogues efficiently rescue the structure and transcriptional activity of mp53 through the formation of a highly stable complex—PANDA.

Here, we disclose, for the first time, a batch of highly resolved crystal structures of PANDA, at approximately 1-3 Å. By analyzing the PANDA crystal structure, we were able to analyze in detail, at the atomic level, how mp53s, such as Structural mp53s are pharmacologically stabilized. In doing so, we discovered a druggable pocket on p53 that can be bound and immobilized by a arsenic, which consequently leads to the global stability of p53 core domain. Based on these findings and through our ultra-large 4C+ screening study, we discovered a vast treasure trove of thousands of clinically relevant mp53 stabilizers (Table 1-Table 6).

### 5.8 Arsenic Compounds with Three or More Cysteine Binding Potential is a Wide-Spectrum and Effective Structural and Functional Rescuer of mp53s

5.8.1 A Class of Rescue Agent Contains Arsenic and can Dramatically Elevate the Tₘ of mp53

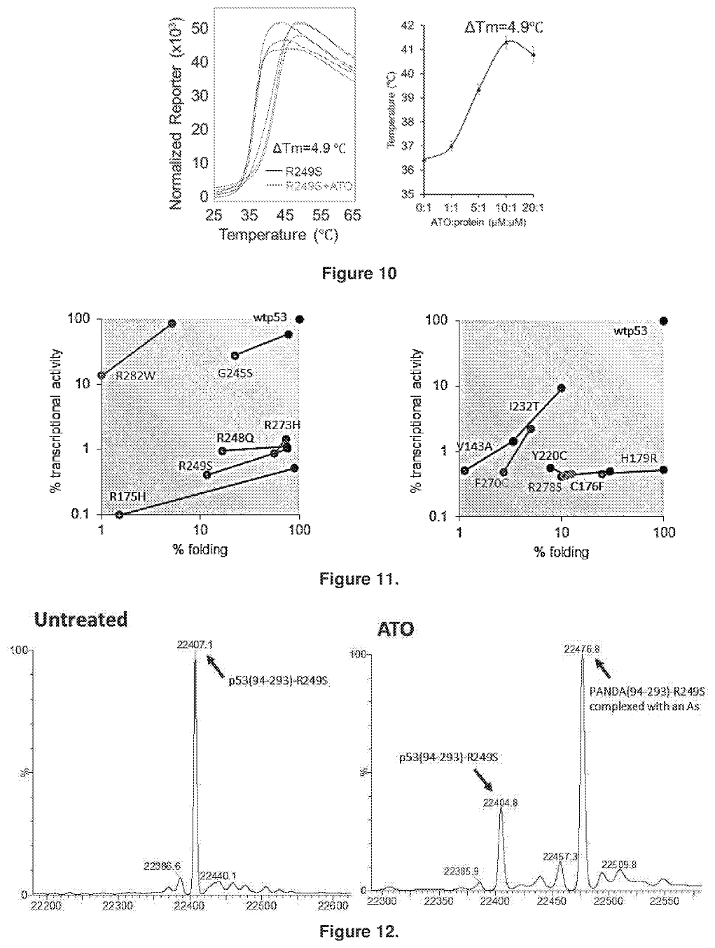

We based our screening method on a hypothesis that there are compounds that can rescue wide spectrum of Structural mp53s by increasing Tₘ, thereby stabilizing mp53. We tested this hypothesis on the four purified recombinant Structural hotspot mp53s and discovered ATO is capable of raising the Tₘ of all four mp53s by approximately 1-8° C., to a level comparable to wtp53. For example, ATO raises the Tₘ of p53-R249S by up to 4.9° C. (FIG. 10). The striking Tₘ enhancement upon ATO treatment indicates mp53 is greatly stabilized.

5.8.2 the Arsenic Rescue Agent is Highly Effective in Rescuing the Structure and Function of mp53s

We systematically and quantitatively determined the structural and transcriptional rescue profile of the rescue agent ATO Before ATO treatment, we confirmed that the folding status of the 6 hotspot mp53s determined by PAb1620 IP efficiency is largely consistent with the thermodynamic stability previously determined by Bullock and colleagues (Bullock et al., 2000) (FIG. 11). We found As₂O₃ has remarkable efficacy in rescuing the structures of all 4 Structural hotspot mp53s tested (p53-R175H, p53-G245S, p53-R249S, p53-R282W) (FIG. 11). We found, for example, the amount of wtp53-like structures when As₂O₃ was added to p53-R175H increased by approximately 50-100 fold, to a level equivalent to 95% of wtp53.

Functionally, As₂O₃ also significantly enhanced the transcriptional activity of the 4 Structural hotspot mp53s on PUMA (FIG. 11) and others. For example, in one of our most successful rescues, we saw As₂O₃ rescued the transcriptional activity of p53-R282W by approximately 20-fold, to a level equivalent to 80% of wtp53. We also saw As₂O₃ rescued the transcriptional activity of p53-G245S efficiently, to a level equivalent to 77% of wtp53.

We further saw that As₂O₃ structurally and transcriptionally rescued Contacting hotspot mp53s, such as mp53-R248Q and mp53-R273H, but at a lower rescue efficiency. It is notable that mp53 structural rescuing efficiency is not necessarily proportionate to the functional rescuing efficiency. While structure of p53-R282W is far from fully rescued, its transcription function is greatly rescued (equivalent to 80% of wtp53 levels). This may be because PAb1620 epitope fails to reflect p53's local structure, for example, the key LSH motif and L3 loop that respond to DNA binding.

In addition to the six hotspot mp53s, As₂O₃ also rescued the other most commonly-occurring mp53s, such as p53-C176F, p53-H179R, p53-Y220C (low efficiency), and p53-P278S (low efficiency) (http://p53.iarc.fr/, IACR) and the representative mp53s with mutations outside of DNA-binding region (p53-V143A, p53-F270C, and p53-I232T) (FIG. 11).

The Structural and transcriptional rescue profile for some of the mp53s are shown in (FIG. 11). These surprising results confirm that As₂O₃ and KAsO₂ represents a wide-spectrum, effective, efficient and robust mp53s rescuing compound.

5.8.3 p53 is Rescued by Binding to a Single Arsenic Atom or Analogue

We further hypothesized a single arsenic atom can bind the key cysteines on mp53 to alter it structures and/or functions. To test this, we created a recombinant mp53(94-293) core with an R249S mutation (“mp53⁽⁹⁴⁻²⁹³⁾-R249S”). We then purified the mp53⁽⁹⁴⁻²⁹³⁾-R249S, incubated the purified mp53⁽⁹⁴⁻²⁹³⁾-R249S with As₂O₃, and measured the molecular weight of the resulting mp53s by mass spectroscopy under denaturing condition.

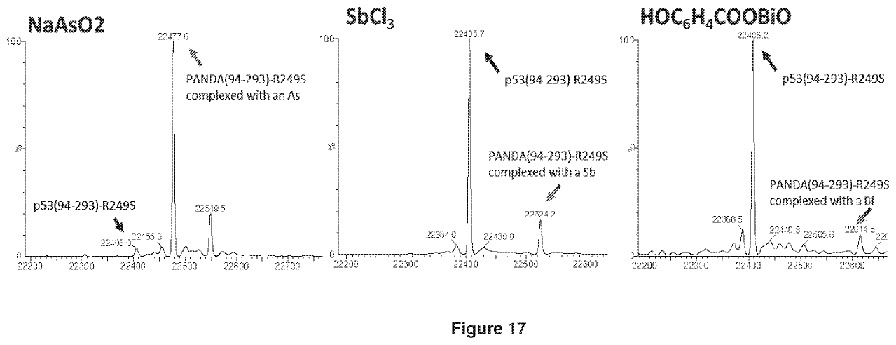

We discovered that the recombinant mp53's molecular weight increased by approximately 72 Daltons (Da) upon incubation, roughly corresponding to the gain of an arsenic atom (74.9) and the loss of 3 protons (FIG. 12). As another example, when we added PANDA Agents identified from our ultra-large scale 4C+ Screening, such as NaAsO₂, SbCl₃, and HOC₆H₄COOBiO to mp53⁽⁹⁴⁻²⁹³⁾-R249S, the molecular weight of the resulting mp53s increased by approximately 72 Da, 119 Da, and 206 Da, respectively (FIG. 17), in accordance with our predictions. This shows a single arsenic atom (or its analogue, including antimony and bismuth) covalently binds to p53.

5.8.4 the Class of Arsenic Rescue Agents Binds to Cysteines on p53 Via its Multiple-Cysteine-Binding Potential

To further understand the interaction between this class of arsenic rescue agents, we turned to the DTP library. We noticed that the DTP library contains many arsenic-containing compounds (n=47). However most of them did not survive in the ‘4C’ Screening. This suggest that the arsenic has to be presented in a correct scaffold to be able to bind p53.

To understand the prerequisite conditions to be an arsenic rescue agent, we compared the 47 arsenic rescue agents, we compared these compounds and their NCI60 cell line inhibition profile. We found that arsenic compounds with three or more cysteine binding potential, such as NSC3060 (KAsO₂, Pearson's correlation 0.837, p<0.01), NSC157382 (Pearson's correlation 0.812, p<0.01), and NSC48300 (Pearson's correlation 0.627, p<0.01), have the most similar NCI60 inhibition profiles as the ATO. Moreover, we found that compounds with bi-cysteine-binding potential also have largely similar NCI60 inhibition profiles as the ATO, though with less extensive (NSC92909, Pearson's correlation 0.797, p<0.01; NSC92915, Pearson's correlation 0.670, p<0.01; NSC33423, Pearson's correlation 0.717, p<0.01).

Moreover, we found that mono-cysteine-binding potential compounds also have significantly similar NCI60 inhibition profiles as the ATO, although the extent was even lower (NSC727224, Pearson's correlation 0.598, p<0.01; NSC724597, Pearson's correlation 0.38, p<0.01; NSC724599, Pearson's correlation 0.553). Summarily, our results showed that compounds with three or more cysteine binding potential, bi-cysteine-binding potential and mono-cysteine-binding potential can selectively inhibit the growth of mp53-expressing cells. Moreover, we showed that the efficiencies among these three classes of arsenic rescue agents decrease with the number of cysteine binding potential it has.

Accordingly, we discovered, for the first time, three separate classes of mp53 rescue compounds, with different rescuing potential. At the very top, those with three or more cysteine binding potential can restore mp53s to near wildtype-like conditions.

Notably, the above mentioned NSC48300 not only has the potential to simultaneously bind 3 cysteines, it also has the potential to simultaneously bind 4 cysteines. This suggests arsenic compound is an efficient mp53 rescuer when it has potential to bind at least three cysteines. It is possible that arsenic compounds with more than three cysteines binding potential can have the same level of rescue efficiency as those compounds with only three cysteines binding potential, because three cysteines were found to be clustered together on p53 (FIG. 5).

### 5.9 Arsenic Selectively Binds PANDA Pocket on Structural mp53s

Since we named the p53 and arsenic analogue complex, PANDA, we decided to follow the nomenclature theme. Based on the crystal structure of PANDA we obtained (described herein), we created the following names. PANDA Cysteine as one of C124, C135, or C141. PANDA Triad as C124, C135, C141 together. PANDA Pocket as the three-dimensional structure centered around PANDA Triad. The PANDA Pocket includes PANDA Triad and directly contacting residues (S116 contacts C124, C275 and R273 contact C135, Y234 contacts C141), residues adjacent to PANDA Triad (V122, T123, T125, and Y126; M133, F134, Q136, and L137; K139, T140, P142, and V143), and residues in 7-A distance to PANDA Triad (L114, H115, G117, T118, A119, K120, S121, A138, I232, H233, N235, Y236, M237, C238, N239, F270, E271, V272, V274, A276, C277, P278, G279, R280, D281, and R282) (FIG. 18). PANDA Agent as the rescue agent capable of forming at least one tight association with the PANDA Pocket. PANDA Agent can be any compound that efficiently stabilizes mp53 by binding potentials to the PANDA Pocket. Preferably, the PANDA Agent enhances Tm of mp53 by 3-100 times of those of PRIMA-1, and/or folds mp53 by 3-100 times of those of PRIMA-1, and/or stimulates mp53's transcriptional activity by 3-100 times of those of PRIMA-1. Preferably, PANDA Agent has at least one cysteine binding potentials, further preferably two or more cysteine binding potential, and further preferably three or more cysteine binding potential. Further preferably, PANDA Agent as compound containing one or more As, Bi or Sb atom. Further preferably, PANDA Agent can be selected from the thousands of compounds listed in Table 1-Table 6, which we have predicted to efficiently bind PANDA Cysteines and efficiently rescue mp53 in situ. More preferably, PANDA Agent is one of the 31 compounds listed in Table 7, which we had experimentally confirmed to rescue mp53's structure and transcriptional activity. More preferably, PANDA Agent include the arsenic analogues such as As₂O₃, NaAs₂, SbCl₃, and HOC₆H₄COOBiO which we confirmed to directly bind p53-R249S (FIG. 12, FIG. 17).

PANDA Core as the PANDA Pocket with a PANDA Agent bounded to it. PANDA as the complex of p53 and PANDA Agent. PANDA is characterized by containing a PANDA Core.

With the identification of a three or more cysteine potential as an important criterion for an efficient PANDA Agent, we started to work on understanding the 3D structure of the PANDA Pocket. In particular, we worked to manipulate the PANDA Pocket to stabilize the mp53.

5.9.1 Remarkable Stability of PANDAs Facilitate Crystallization of PANDA and Identification of the PANDA Pocket

While the core of the wtp53 has been previously crystalized, it is notoriously difficult to crystalize the core of a Structural hotspot mp53. This is because Structural hotspot mp53s have very low stability.

However mp53s can be artificially stabilized by introducing four SSSMs (M133L, V203A, N239Y, and N268D), resulting in a quadruple mutant p53-QMs. The four SSSM elevates Tₘ of the p53 by 5.2° C. This enhanced stability facilitate crystallizations, and many Structural mp53s, including hotspot mp53-G245S and mp53-R282W and non-hotspot mp53-V143A and mp53-F270L, were resolved.

Our PANDA is remarkably stable.

In fact, PANDA Agent can elevate the Tₘ of a mp53 to a level comparable to the QMs (FIG. 10).

The remarkable stability of our PANDAs can enable us to crystalize Structural hotspot mp53s, including p53-R249S and As in a batch of conditions and p53-G245S and p53-R282Q without SSSM.

Based on our PANDA crystals, we confirmed our mass spectroscopy results that a single arsenic (or analogue) atom covalently binds to three cysteines. These three cysteines are: C124, C135, and C141 (each a “PANDA Cysteine” and together a “PANDA Triad”) within the PANDA Pocket.

5.9.2 the Most Effective PANDA Agent Binds to the Highly Inert PANDA Triad Despite Other More Accessible Cysteines are Available and Despite Alternative Tri-Cysteine Metal Binding Site is Available.

To understand the MOA of the PANDA Triad, we knew we need to find out what are the PANDA Triad and where are they located. In addition, one of the major challenges for a cysteine binding compound in clinical studies is off-targeting of undesired cysteines (Joerger and Fersht, 2016; Kaar et al., 2010). Accordingly, it is crucial to map out the cysteines of p53 responsible for PANDA Agent binding.

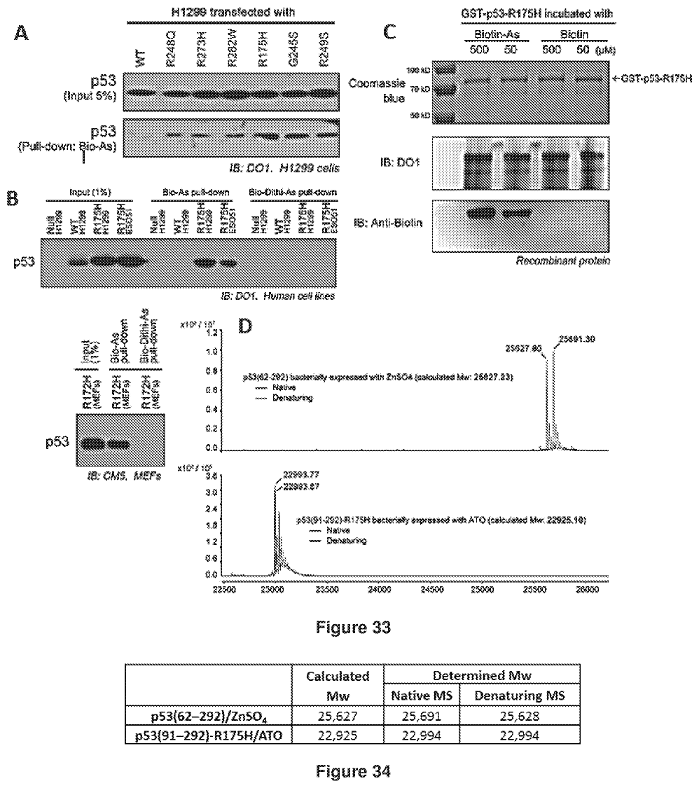

We listed all of the 10 cysteines on p53 (FIG. 5) and investigated their selectivity for arsenic. Since arsenic must bind to cysteines on p53 (FIG. 33B), we expect PANDA Cysteines to localize to the outer surfaces of p53, exposed to the solution. Consistent with a previous in-silico study (Kaar et al., 2010), we discovered that C182 and C277 are highly exposed on the surface of p53 (FIG. 5). This is consistent with Bauer, which showed that a mp53 stabilizer, PK11000, binds C182 and C277 (one PK11000 molecule binds one Cysteine) (Bauer et al., 2016). To our surprise, unlike the highly exposed and highly reactive C277 and C182, we found two of the PANDA Cysteines to be highly inert. In fact, the PANDA Cysteines C135 and C141 are deeply buried. This is consistent when we correlate the location of the PANDA Pocket with a reported p53 crystal that shows PANDA Pocket was not alkylated when soaking the crystal with cysteine binding compound (Kaar et al., 2010).

In our crystal, in the presence of arsenic, we found that the arsenic selectively bound the highly inert PANDA Cysteines (C135 and C141) in vivo to form the PANDA Core on PANDA (FIG. 14). To emphasize, the PANDA crystal which enabled us to resolve the PANDA Pocket and PANDA Cysteines, was formed in vivo by treating mp53 expressing bacteria with ATO. These data definitively show that, contrary to normal expectations, in living organisms, arsenic has a high affinity for the PANDA Cysteines, specifically selects them over more readily available cysteines, such as C182 and C277.

Our results also show this is the case in vitro. When we soaked a mp53 crystal with arsenic, we produced a PANDA crystal, that once again demonstrated that arsenic selected for and bound to the highly inert PANDA (FIG. 14). More notably, under this particular condition, the PANDA Triad had very restricted accessibility and reduced structural plasticity. Despite this, arsenic still found and bound to the PANDA Triad, providing convincing evidence that arsenic is also highly selective to PANDA Cysteines in vitro.

Based on our crystal structures, we reasoned that arsenic is attracted by the inert PANDA Cysteines on PANDA Pocket over reactive cysteines that are more readily available, such as C277 and C182, may be due to arsenic's prefers to bind tri-cysteines clusters over bi-cysteine dusters and mono-cysteines. Consistent with this theory, it has been reported that arsenic prefers to bind Zinc finger domains containing 3 and 4 cysteines (CCCC-Zinc finger and CCHC-Zinc finger) rather than CCHH-Zinc finger domain, which contains 2 cysteines (Zhou et al., 2011). Accordingly, we evaluated arsenic's binding potential to other tri-cysteine clusters, such as the zinc region composed of C176/C238/C242 (“Zinc Region”).

There are many reasons that this Zinc Region is an ideal site for arsenic. First, the Zinc Region harbors three of the mp53 mutation hotspots, namely, R175, G245, and R249. These mutation hotspots are more efficiently structurally rescued by As₂O₃ as compared to other mp53s, such as mp53-R282W (FIG. 11). Second, zinc readily dissociates from mp53-R175H (Butler and Loh, 2003; Loh, 2010) and we previously showed that arsenic can occupy the Zinc binding site in proteins such as promyelocytic leukemia protein (“PML”) (Zhang et al., 2010). ATO can bind to PML-RARα in situ and can clinically cure acute promyelocytic leukemia (“APL”), the only malignancy that can be definitely cured by targeted therapy (Hu et al., 2009; Lo-Coco et al., 2013). Third, our in silico docking studies suggest that the Zinc Region tri-cysteines C176/C238/C242, which are in close proximity to each other spatially, form an excellent pocket for arsenic.

Surprisingly, despite these promising characteristics, our studies show that the arsenic atom did not bind to the Zinc Region on our PANDA crystal structure. This is the case even when we depleted zinc atoms using EDTA to promote arsenic binding (data not shown). Instead, our studies show that arsenic binds to the deeply buried Site on the PANDA Pocket. Our results show that arsenic's cysteine selectivity is nontrivial. Selectivity of arsenic to p53's cysteines is not simply based on accessibility of an individual cysteine, and it is not simply based on the presence of tri-cysteine clusters. This is true even when a ti-cysteine site can attract and form bonds with other metallic elements, such as zinc.

Our results emphasize that the PANDA Triad (C124/C135/C141) and PANDA Pocket we discovered are special and unique for arsenic and its analogues.

### 5.10 Arsenic Atom Freely Enters into L1-S2-S3 Pocket, Further Passes Through L1-S2-S3 Pocket, and Reaches and Stays in PANDA Triad

The 3D structure of p53 has been solved for over 24 years and hundreds of different-size pockets can be visually identified on its surface. However, none of them are experimentally tested to be functional. Here, we identified the PANDA Triad to locate below a pocket spanning L1 loop, S2 sheet, and S3 loop of p53, which we designated as L1-S2-S3 pocket. This L1-S2-S3 pocket is previously named as L1-S3 pocket or L1/S3 pocket (Joerger and Fersht, 2016; Wassman et al., 2013).

Many of the previously reported compounds were predicted to bind to C124 of L1-S2-S3 pocket in a computer modelling (Joerger and Fersht, 2016; Wassman et al., 2013). Most of the agents used clinically contain about 10-100 atoms, as are the previously reported mp53 rescue compounds. The L1-S2-S3 pocket is relatively small so that the previously reported mp53 rescue compounds can only enter into it occasionally, only when it is open (FIG. 15).

Our single atom PANDA Agent, such as the single arsenic atom, is fundamentally different from any of clinically using agents and the previously reported mp53 rescue compounds by the fact that it is just a single atom. Arsenic atom is smaller than any of the reported mp53 rescue compounds by one or two orders of magnitude (about 1/10- 1/100 size of reported compounds). It is so small that it can freely enter into L1-S2-S3 pocket at any time, even when it is closed (FIG. 15). Arsenic atom is also fundamentally different from the previously reported mp53 rescue compounds by it does not stay in L1-S2-S3 pocket, but rather pass through it. Arsenic atom is so small that it can freely pass through L1-S2-S3 pocket and further enter into the PANDA Triad, an extremely small pocket that can only accommodate one atom.

### 5.11 Immobilizing PANDA Pocket is Sufficient to Stabilize mp53s

We further discovered that arsenic stabilizes mp53 by immobilizing PANDA Pocket. Taking advantage of the atom-level rationale of how mp53 is stabilized by arsenic, we further discovered that PANDA Pocket is in fact a key switch that controls mp53 stability. More importantly, it can be utilized to identify p53 rescue agents (or PANDA Agents).

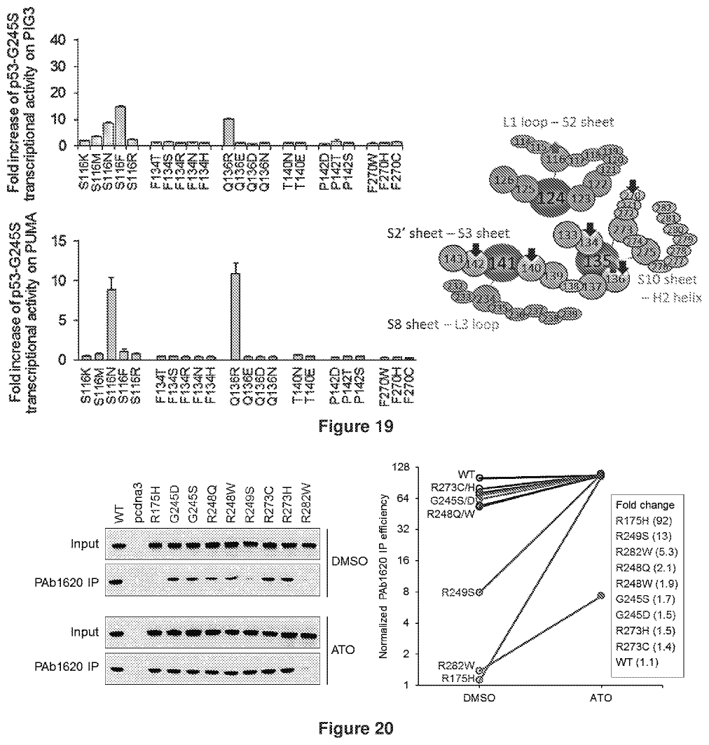

By analyzing the intramolecular interaction between the residues of PANDA Pocket (FIG. 18), we predicted a group of key residues, including S116, F134, Q136, T140, P142, and F270 to play significant role in controlling the stability of PANDA Pocket on p53 (FIG. 19). By introducing a batch of artificial mutations on these residues, we found, for example, S116N, S116F and Q136R can significantly rescue the transcriptional activity of p53-G245S on PIG3 (FIG. 19). In addition, we found residues, such as S116N and Q136R can rescue the transcriptional activity of p53-G245S on PUMA (FIG. 19). Thus, we discovered that S116N, S116F and Q136R can act as SSSMs by mimicking PANDA Agent to rescue mp53. Our findings confirms that immobilizing PANDA Pocket by, either PANDA Agents or rationally designed SSSM, is sufficient to stabilize mp53.

Many SSSMs were previously identified by sequence evolutional analysis or function-guided screening in the past two decades (Baroni et al., 2004; Nikolova et al., 1998). Interestingly the majority of reported SSSMs locate near PANDA Pocket. Moreover, our rationally designed and discovered batch of SSSMs efficiently rescued Structural mp53, stabilizing the PANDA Pockets and demonstrating the discovery of a novel method of using arsenic compounds to rescue Structural mp53s by immobilizing PANDA Pocket.

The L1 loop (F113-T123) on the top of PANDA Pocket is particularly interesting because it is a coldspot for cancer mutation (IACR, http://p53.iarc.fr/TP53SomaticMutations.aspx) and it is the most dynamic DNA-binding element (Lukman et al., 2013). Notably, mutations on these residues frequently boost p53's function, again supporting our findings that manipulating PANDA Pocket is able to rescue mp53.

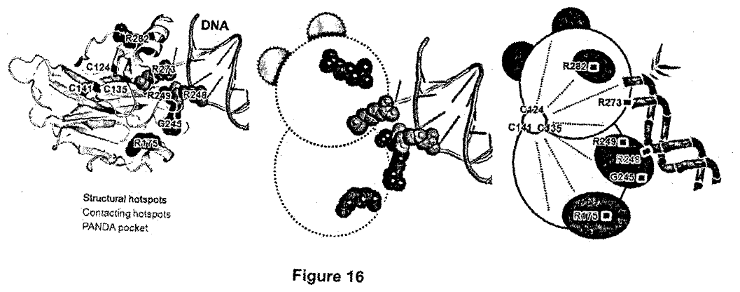

In brief, we discovered a PANDA Pocket that is a key switch in controlling mp53 stability. PANDA Pocket locates at the “dorsal end of PANDA” (FIG. 16). It is known that grasping mammalian neonates by the dorsa is able to induce a dorsal immobility response (DIR) and calm the infants of human, mouse, lion, and others (Esposito et al., 2013). Manipulating PANDA Pocket can rescue mp53's wildtype structure and transcriptional function. PANDA Pocket-binding compounds can potentially act as PANDA Agents (mp53 rescue agents). By discovering the key role of PANDA Pocket in rescuing mp53, we have now expanded our 4C Screening to a 4C+ Screening for additional PANDA Agent that contain As, Sb, or Bi, but nevertheless can form at least one tight bond to PANDA Pocket, and further suggest other non-As, Sb, and Bi compounds can also serve as efficient PANDA Agents.

### 5.12 the Discovery of Thousands of Efficient, Effective and Wide-Spectrum Mp53 Rescuers

With insights of the PANDA Pocket, extremely high efficiency of tri-cysteine binding arsenic in rescuing mp53s, and the MOA of arsenic, we conducted an ultra-large C4+ Screening. We predicted thousands of compounds have the potential to efficiently bind 3 or more cysteines and thus act as efficient mp53 rescuers (Table 1-Table 6). We randomly selected some compounds from Table 1-Table 6, together with some compounds with only one or two cysteine-binding potential and experimentally confirmed 31 mp53 recusing agents with key supporting data (rescue mp53's structure; rescue mp53's transcriptional activity). They are listed in Table 7.

We discovered that Sb and Bi compounds, like arsenic compounds, can also rescue mp53s (Table 7). We confirmed in mass spectroscopy that As, Sb and Bi can directly and covalently bind mp53 (FIG. 17).

We further discovered that organic As, Sb, and/or Bi containing compounds can also efficiently rescue mp53s (Table 7).

We further discovered that both 3-valence and 5-valence As, Sb, and/or Bi containing compounds can efficiently rescue mp53s.

We further discovered one of the prerequisite of being an efficient mp53 rescuer is tri-cysteine binding capacity. For example, NSC43800 (which can simultaneously binds 3-4 cysteines) rescues the transcriptional activity of mp53 with higher efficiency than NSC721951 (which can only bind 1 cysteine).

Worth noting here is that the ability of organic As, Sb, and/or Bi to efficiently rescue mp53 through the PANDA Pocket, despite the limited space in the PANDA Triad is unlikely to accommodate an organic compound, particularly those with a benzene, suggest that the cysteine binding potential of arsenic is so strong that it can robustly insert into the small space in PANDA Triad, probably leaving bulky organic groups, such as benzenes, in the L1-S2-S3 pocket and outside of the PANDA Triad. Moreover, it is possible that more profound influence on the mp53's structure may be going on when organic arsenic is bounded.

We further found that As, Sb, and/or Bi compounds with mono-cysteine binding potential (e.g.: NSC721951) or bi-cysteine binding potential (e.g.: NSC92909) can also rescue mp53's structure and transcriptional activity. When compared to compounds with cysteine binding potential, we found that compounds with three or more cysteine binding potential have the highest rescue efficiency, followed by compounds with bi-cysteine binding potential, and followed by compounds with mono-cysteine binding potential (FIG. 64-FIG. 68).

The discovery of compounds containing Bi and/or Sb, and organic As, Sb, and/or Bi compounds with mp53 rescue capacity has tremendous clinical value because these compounds generally have lower toxicities than inorganic As compounds in the body.

### 5.13 Clinical Trials

We conducted a small scale trial treating patients harboring ATO-rescuable mp53s. We conclude that ATO is a PANDA Agent with definite effectiveness and mp53 selectivity Based on current finding, two large-scale multi-center prospective trials on AML/MDS patients have been carried out (NCT03381781 and NCT03377725).

### 5.14 ATO Strongly Promotes Proper Folding of the Unfolded Population of p53 Under a Wide Range of Settings and Independent of a Wide Array of Factors

Since our 4C Screening identified ATO as a PANDA Agent, we studied whether ATO directs proper folding of the unfolded population of p53. Using an antibody specific to the properly folded wtp53, PAb1620, we immunoprecipitate (“IP”) properly folded p53s. Consistent with our predictions, we found wtp53 and Contacting mp53s, such as p53-R273H/C, to be largely folded (See FIG. 20). In contrast, we found Structural mp53s, such as p53-R175H, p53-G245S/D, p53-R249S, and p53-R282W, and some Contacting mp53s, such as R248Q/W, to be unfolded to vary degrees (see, FIG. 20). However, after ATO treatment, the unfolded population of all p53s folded with a remarkable efficiency, and with the exception of p53-R282W, all properly folded to a level comparable to wtp53 (see, FIG. 20). Among these, p53-R175H had the most dramatic change, where the percent of properly folded p53s increased by as much as 92 times (See FIG. 20). Even the folded population of wtp53 and p53-R273H/C detectably increased with ATO treatment, demonstrating that ATO is such a strong agent, it can further promote folding of the predominantly folded population of wtp53 and p53-R273H/C (see, FIG. 20).

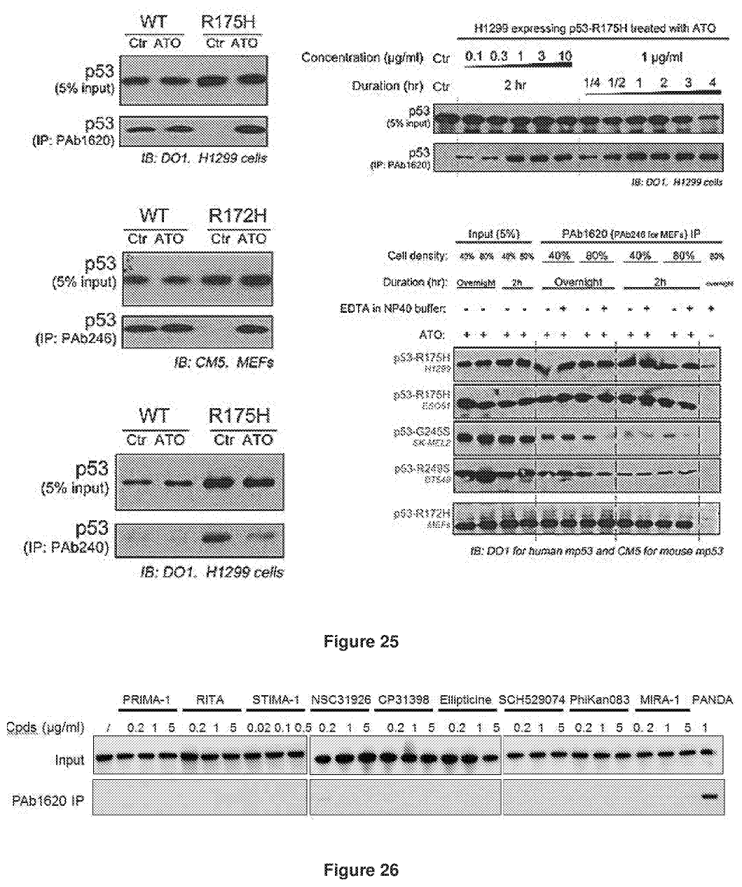

The ability of ATO to fold mp53 was further supported using two other p53 conformation-specific antibodies, the PAb246 antibody specific to properly folded p53 (for mouse p53) and the PAb240 antibody specific to unfolded p53 (FIG. 25).

We also carefully characterized the ATO mediated mp53 folding under a variety of conditions. We found that 0.1 μg/ml of ATO was sufficient to properly fold some mp53s (FIG. 25). Further, the folding appeared to be instantaneous, because it took only 15 min for ATO to enter the cells and properly fold p53-R175H. (See FIG. 25) In addition, ATO mediated folding was largely independent of many factors, including, the cell type (for example, all cells tested, including MEF, H1299, ESO51, SK-MEL2, and BT549, were responsive), cell confluence during treatment (for example, all confluency tested, including at 40% and 80% confluency, were responsive), duration of treatment (for example, all durations tested, including 2 hours and overnight, were responsive), mp53 source (for example, all source tested, including human mp53s and mouse mp53s, were responsive), and the type of IP buffer (for example, all buffers tested, whether with or without EDTA, were responsive). (See FIG. 25).

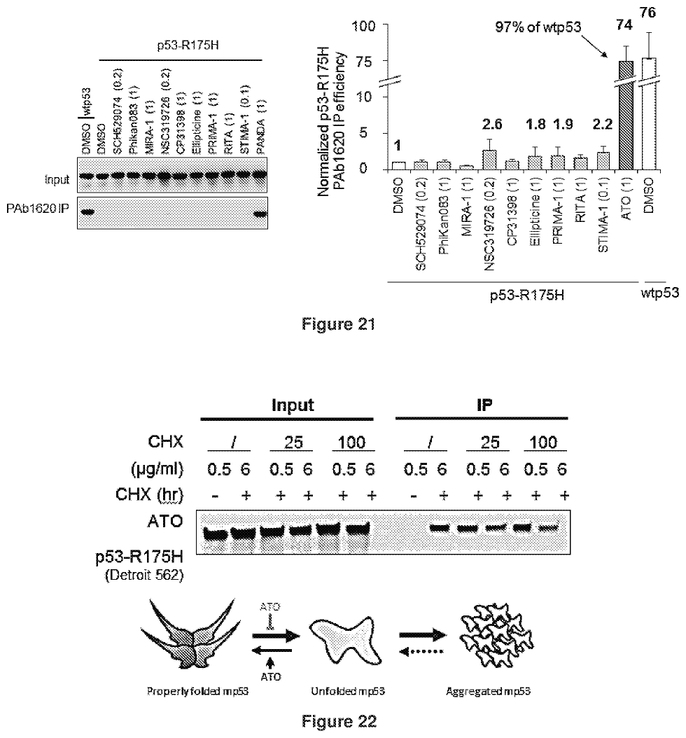

One of our focuses is p53-R175H, the most frequent individual mp53 found in cancers and the most representative Structural mp53 (Freed-Pastor and Prives, 2012). We carefully compared the ATO mediated mp53 folding efficiency to previously reported rescue compounds such as PRIMA-1, NSC319726, Ellipticine, STIMA, PhKan083, and others (FIG. 26). One of the reasons we chose these compounds is because there is still considerable debate on the efficiency of these reported rescue compounds (Joerger and Fersht, 2016; Muller and Vousden, 2013, 2014). To prepare for our studies, we carefully titrated the treatment conditions for the reported compounds (see FIG. 26) and optimized the conditions for our studies (See FIG. 21).

We observed an 1.9 times increase in the properly folded population of p53-R175H as measured by PAb1620 upon PRIMA-1 treatment (see FIG. 21). This result is comparable to prior studies showing a 3 times increase for purified recombinant GST-p53R175H and a 1.46 times increase for p53-R175H derived from SKOV-His-175 cell lysates (Bykov et al., 2002). NSC319726, Ellipticine and STIMA also had a similar effect on p53-R175H, resulting from 1.8 times to 2.6 times increase in the properly folded population of p53-R175H. PhiKan083 did not efficiently fold p53-R175H, which is probably because it is a p53-Y220C specific rescuer (Boeckler et al., 2008).

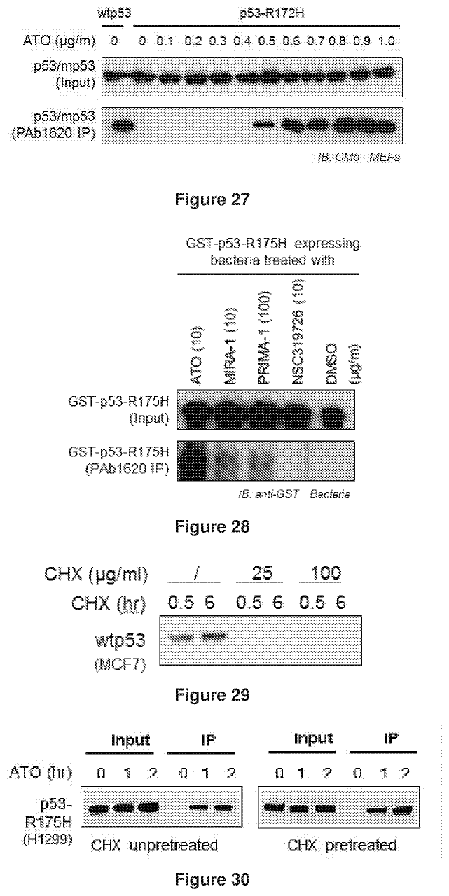

Very strikingly, we observed ATO increased the properly folded population of human p53-R175H by about 74 times, as measured by PAb1620. (See FIG. 21). At this level, p53-R175H structure has been restored to a level comparable to the wtp53 (or to approximately 97% of the wtp53 levels). (See FIG. 21). In addition to restoring human p53s, we observed ATO nearly completely restored the population of unfolded mouse p53-R172H to that of wildtype level (See FIG. 27). Furthermore, we found ATO also properly folded bacterial recombinant p53s robustly in vivo at a rate substantially more efficient than all the previously reported compounds we tested. For example, FIG. 28 shows adding ATO to recombinant GST-p53-R175H in bacteria substantially increased the epitope for properly folded p53 (i.e. the PAb1620 epitope). Furthermore, the level of ATO-mediated p53 folding was substantially higher than known rescue compounds such as MIRA-1, PRIMA-1, and NSC319726. (See FIG. 28).

### 5.15 ATO Broadly Promotes Mp53 Stabilization and Prevents Mp53 Aggregation

Because of the low kinetic stability of mp53, others have proposed that a compound rescuing the structure of mp53 must act immediately upon mp53 translation (Joerger and Fersht, 2007). We tested this hypothesis by pre-treating cells with the translation-inhibitor cycloheximide (“CHX”), so that p53-R175H stays at its unfolded or denaturing status. We also confirmed that the CHX pre-treatment efficiently blocked p53 translation in our system (See FIG. 29). Remarkably, we observed that even with CHX pre-treatment, ATO can still efficiently and properly fold endogenous p53-R175H in cells (see FIG. 22) and exogenous p53-R175H in H1299 cells (see FIG. 30). Our results suggest that ATO can properly fold even denatured mp53s (FIG. 22, 3D structure).

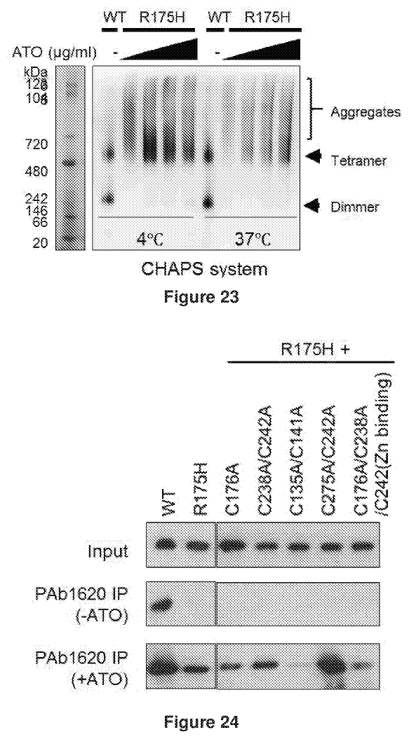

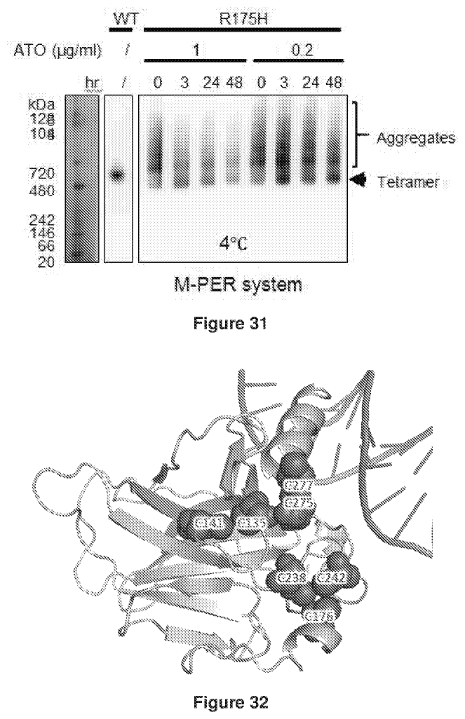

Others have reported that stabilizing p53 in its native state can inhibit p53 aggregation (Bullock et al., 1997). Here, we discovered that ATO mediated stabilization reduces the number of p53-R175H aggregates (See FIG. 23 and FIG. 31). We confirmed this in native PAGE using both the CHAPS system (FIG. 23) and the M-PER system (FIG. 31). Accordingly, we confirmed that ATO mediated restoration converts mp53s to its native, properly folded, and stabilized state and prevents mp53 aggregation.

### 5.16 R175-Distant C135/C141 Cluster is Involved in as Binding

Arsenic was reported to bind multiple closely spaced cysteines rather than single cysteine on peptides (Donoghue et al., 2000). Accordingly, we explored As-mediated mp53 folding. We studied all of the three pairs of cysteines, including C135/C141, C238C242, and C275/C277, and the cysteine neighboring R175, namely C176 (see FIG. 32). Surprisingly, we identified, for the first time, that alanine mutations on the R175-distant C135/C141 duster, but not neighboring C176 or C238/C242, greatly interferes with ATO-mediated folding of p53-R175H of PANDA Core and PANDA. (See FIG. 24).

The characteristics of ATO mediated folding include:

(a) able to properly fold all tested Structural hotspot mp53s with a range of efficiency, including high to extremely high efficiency;

(b) instant folding (<15 min);

(c) folding is independent of cell types and treatment contexts, including resistant to EDTA in IP buffer;

(d) folding is much more efficient than any of the reported compounds;

(e) p53-R175H is almost fully restored as measured by the PAb1620 epitope;

(f) efficient for both human mp53 and mouse mp53;

(g) works in both mammalian cells and bacterial cells;

(h) can fold mp53 that has been previously unfolded;

(i) inhibits mp53 aggregation; and

(j) Cys135 and Cys141 are involved in As-mediated mp53 folding.

### 5.17 As Binds to p53 to Form PANDA Irrespective of the Source of p53s

Since As is able to properly fold Structural mp53s rapidly and effectively, we studied whether As directly interacted with p53-R175H. We treated p53s with biotin-labeled As (“Bio-As”) (Zhang et al., 2010), pulled down Bio-As to determine any As associated complexes. (See FIG. 33) We discovered, for the first time, that As can bind mp53. (See FIG. 33). We further found that, among the six well known mp53 hotspots (R175H, G245S, R249S, R282W, R248Q, and R273H), more Structural mp53 (e.g.: p53-R175H, p53-G245S, p53-R249S, and p53-R282W) forms PANDA as compared to wtp53 and Contacting mp53 (e.g.: p53-R248Q and p53-R273H). (See FIG. 33). Our detailed study of p53-R175H further showed that As, such as Bio-As, can rapidly and effectively bind to the mp53s irrespective of the source. For example, exogenous p53-R175H in H1299, endogenous p53-R175H in ESO51, and p53-R172H from mouse embryonic fibroblasts all can bind to Bio-As to form PANDA. (See FIG. 33).

### 5.18 Arsenic's Selectivity Among p53s

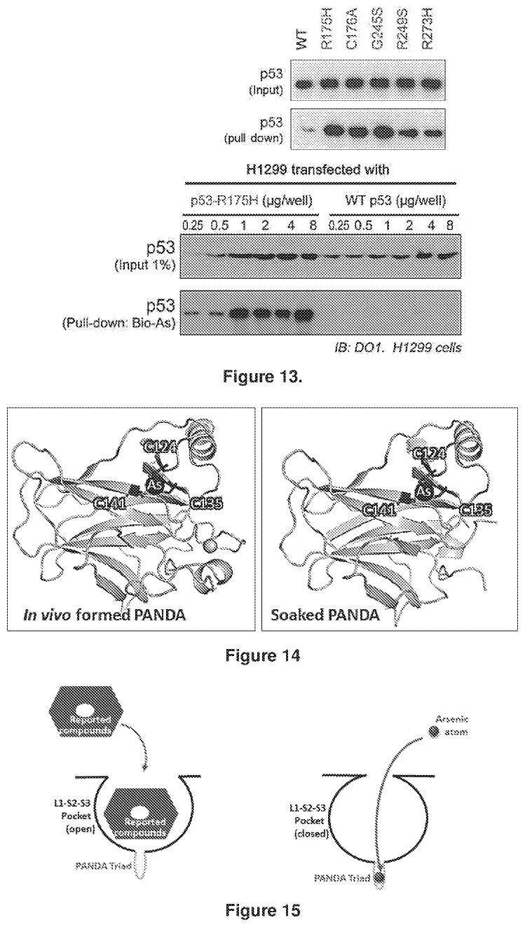

We further tested the selectivity of arsenic among cellular p53s. We labelled arsenic atom with biotin to form biotin-As and incubated the biotin-As with cells expressing a variety of p53s. We then lysed the cells and pulled down biotin-As for by immunoblotting. Our results show that biotin-As prefers to bind Structural mp53s rather than Contacting mp53s (FIG. 13). Interestingly, biotin-As binds to wtp53 with even lower efficiency than those of Contacting mp53 (FIG. 13).

When the binding efficiency between biotin-As and p53-R175H or wtp53 is carefully titrated, it was found that biotin-As bound p53-R175H with at least 10 times higher efficiency than wtp53 (FIG. 13).

The biotin-As relevant data needs to be carefully evaluated because a bond for cysteine binding on biotin-As is occupied by biotin, and thus the results may not precisely reflect the selectivity of ATO on wtp53 and mp53s. These data implies a potential arsenic selectively binding unfolded mp53s rather than folded mp53s and wtp53s.

### 5.19 Cysteine is Involved in As Mediated PANDA Formation

We further discovered that cysteine is involved in As mediated PANDA formation. For example, we found that treatments with Bio-Dithi-As, a compound where As is protected by dithiols and cannot bind to cysteines (Heredia-Moya and Kirk, 2008), cannot pull down p53-R175H. (See FIG. 33). This supports the cysteines of p53, such as mp53, are involved in PANDA formation.

### 5.20 Elemental As Directly and Covalently Interacts with p53s

To further characterize PANDA, a fusion protein combining a recombinant GST and the full-length p53-R175H (“GST-p53-R175H”) was expressed in bacteria, purified, and then incubated with Bio-As in vitro. Remarkably, using this method, we discovered an As-Biotin-GST-p53-R175H complex that survived protein denaturation and protein electrophoresis, such as SDS-PAGE. (See FIG. 33). This supports that As directly and covalently interacts with p53s such as mp53.

### 5.21 Elemental As Directly and Covalently Interacts with the Core Domain of p53 at 1:1 as to Protein Ratio

We further determined the direct and covalent interaction between the core domain of p53 and compounds containing As, Sb, or Bi. We expressed a recombinant wtp53 core (“wtp53(62-292)”) and a recombinant mp53 core (“mp53(91-292)-R175H”) in the presence of ZnSO₄ and ATO, respectively. We then purified these core fragments and determined their molecular weight by mass spectroscopy (“MS”). In their native conditions, the molecular weight of wtp53(62-292) and mp53(91-292)-R175H are higher than expected, at approximately 64 Da and approximately 69 Da higher, respectively. This supports the formation of wtp53(62-292)/Zn complexes and mp53(91-292)-R175H/As complexes at 1:1 p53:metal ratio. (See FIG. 33 and FIG. 34). However, under denaturing conditions, we found the mass of wtp53(62-292)/Zn to drop by 63.5 Da, but the mass of mp53(91-292)-R175H/As did not. (See FIG. 33 and FIG. 34). This further confirms that As covalently binds to p53s, such as mp53 (see FIG. 33).

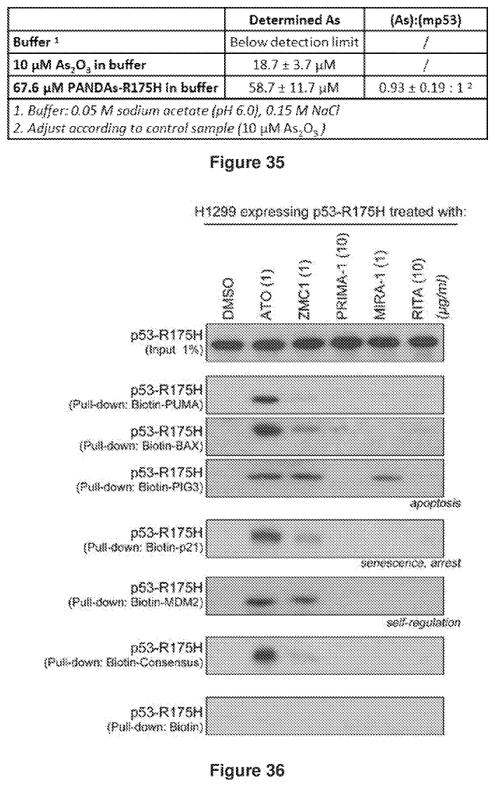

We further confirmed that As binds to p53 in an 1:1 ratio by inductively coupled plasma mass spectroscopy (“ICP-MS”). For example, our results not only show that As binds covalently to p53s, but that each p53 binds to approximately one As atom (0.93±0.19 As per p53). (See FIG. 35).

The characteristics of PANDA-forming reactions include the following:

(a) prefers to bind Structural mp53;

(b) works for both human mp53 and mouse mp53;

(c) works in both mammalian cells and bacterial cells;

(d) works in vivo (in cells) and in vitro (in reaction buffer)

(e) mp53 cysteine(s) are involved;

(f) reaction is in a 1:1 molar ratio between mp53 and As atom

(g) direct reaction; and

(h) covalent reaction.

### 5.22 PANDA Regains Wildtype DNA-Binding Ability and Wildtype Transcriptional Activity

Since As mediates PANDA formation and efficiently rescues the structure of p53s, we further examined the DNA binding and transcriptional activity of PANDA.

5.22.1 PANDA Regains Wildtype DNA-Binding Ability

We biotin labelled a wide range of p53 targets and p53-binding consensus sequence and found that a wide range of PANDAs, including PANDA formed from p53-R175H (“PANDA-R175H”), can bind a wide range of p53 targets. For example, we showed that PANDA, including PANDA formed from p53-R175H can bind to MDM2, which is involved in p53 self-regulation; CDKN1A, which encoding p21 protein and is involved in senescence, invasion, metastasis, cell stemness and cell cycle arrest; PIG3, which is involved in apoptosis; PUMA, which is involved in apoptosis; BAX, which is involved in apoptosis; and the p53-binding consensus sequence. (See FIG. 36). We further found that PANDAs have significantly higher affinities to these p53 targets as well as p53-binding consensus sequence than their corresponding mp53s (i.e. when PANDA is not formed); and PANDAs formed with As has significantly higher affinities to these p53 targets as well as p53-binding consensus sequence than when mp53s are treated with other rescue agents such as ZMC1, PRIMA-1, MIRA-1, or RITA.

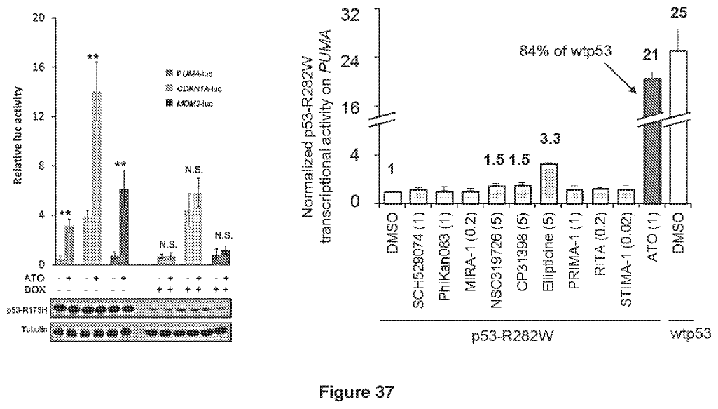

When we measured the ability of As₂O₃ to rescue p53 transcriptional activities in luciferase assays, we discovered PANDAs significant enhanced the transcription activities p53 targets, such as PUMA, CDKN1A and MDM2 in the luciferase assay. (See FIG. 37). The enhanced luciferase signal is largely mp53 dependent because the enhancement was greatly abolished by switching off p53-R175H using doxycycline (“DOX”). We also discovered that a dramatic enhancement of transcriptional activity of PANDA-R282W on PUMA promoter (21 time increase, equivalent to 84% of wtp53 levels) (see FIG. 37) and PANDA-G245S on PIG3 promoter (nearly 3 times increase, equivalent to 77% of wtp53 levels) (see FIG. 41).

Comparing to other rescue agents, we found that ATO mediated PANDA formation is a far more superior rescue agent for p53 transcriptional activity. In particular, we found the other rescue agents measured at negligible for SCH529074, negligible PhiKan083, negligible for MIRA-1, negligible for PRIMA-1, 1.5 times for NSC319726, 1.5 times for CP31398, negligible for RITA, negligible for STIMA-1 and 3.3 times for Ellipticine and 21 times for ATO. (See FIG. 37 and FIG. 41).

5.22.2 PANDA Dramatically Increases Wildtype Transcriptional Activities

In particular, we found the other rescue agents measured at negligible for SCH529074, negligible PhiKan083, negligible for MIRA-1, negligible for PRIMA-1, 1.5 times for NSC319726, 1.5 times for CP31398, negligible for RITA, negligible for STIMA-1 and 3.3 times for Ellipticine and 21 times for ATO. (See FIG. 37 and FIG. 41). In contrast PANDA dramatically increases wildtype transcription activities.

PANDA dramatically increases p53 downstream mRNA production levels in cells expressing exogenous mp53s or endogenous mp53s. Adding ATO to H1299 cells expressing exogenous p53-R175H can dramatically stimulate the levels of p53 downstream mRNAs, including MDM2, PIG3, PUMA, CDKN1A, and BAX in 24 hr. Expectedly, the wtp53-stimulating Nutlin significantly enhanced PUMA, PIG3, CDKN1A and MDM2 mRNA levels in HCT116 cells expressing wtp53.

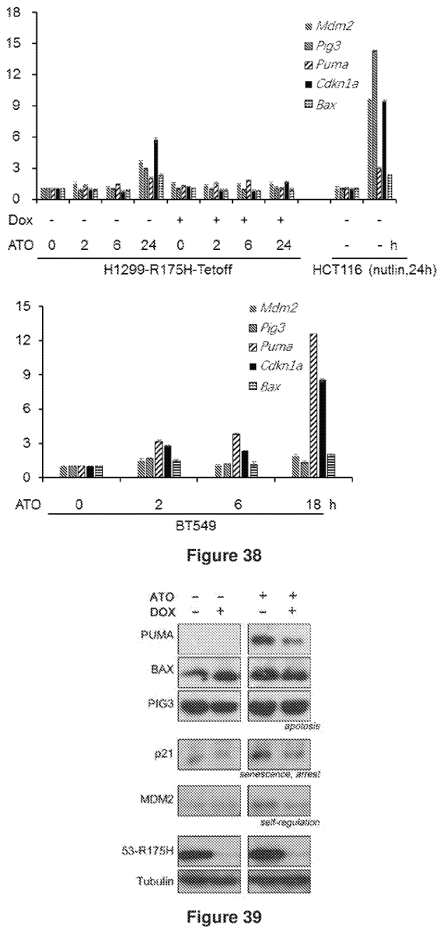

Adding ATO to BT549 cells expressing endogenous p53-R249S can dramatically stimulate the levels of p53 downstream mRNAs, including PUMA and CDKN1A. (FIG. 38).

At the protein level, PANDA can dramatically increase p53 downstream protein production levels in cells expressing mp53. For example, by adding ATO to cells that express mp53s, such as H1299 cells, which expresses p53-R175H, we detected an increase in p53 targets (i.e. downstream proteins), such as PUMA, BAX, PIG3, p21, and MDM2 (See FIG. 39). PANDA formation is necessary during the upregulations of these proteins because DOX induced mp53 depletion largely abrogated these upregulations. In addition, we found ATO to significantly upregulated PUMA protein in HCT116 cells expressing Structural mp53s including p53-R175H, p53-R249S, or p53-R282W through the formation of PANDA (FIG. 42).

### 5.23 PANDA is a Tumor Suppressor In Vitro

We further discovered, for the first time, that PANDAs, such as PANDA-R175H, not only regain wtp53 transcriptional activity, but that they regain wtp53 tumor suppressive abilities in vitro and in vivo, including in xenograft models. We found that combining ATO with p53-R175H expressing cells dramatically increased the sensitivity of mp53 expressing cells, such as H1299 cells, to cell death, suggesting that the formed PANDA-R175H plays a tumor-suppressive role in the cells by suppressing cell growth (See FIG. 44). In addition, we found that combining ATO with p53-R175 expressing cells, such as H1299, also significantly inhibited colony formation of these cells and in a largely p53-R175H dependent fashion, further suggesting that the formed PANDA-R175H plays a tumor-suppressive role by suppressing colony formation. (See, for example, our colony assay results in FIG. 44). Similar results were observed in mouse embryonic fibroblasts (MEFs), in which the presence of PANDA-R172H conferred sensitivity to ATO treatment in both cell viability assays and colony formation assays. (See FIG. 49). In contrast, when p53 is absent (i.e. in p53 null cells), PANDA cannot form and accordingly, these cells are more resistant to ATO treatment (FIG. 49). These demonstrate that ATO binds mouse p53-R172H to form the tumor suppressor PANDA-R172H and inhibits cell growth and colony formation. Taken together, our results show that ATO transforms mp53s, such as p53-R175H, into a tumor-suppressive PANDA in vitro.

To test whether ATO targets Structural mp53 to inhibit malignancies, we applied ATO to 10 cell lines with differing p53 status, including wtp53, p53⁴ (null), truncated p53, p53-R249S, p53-R175L, and p53-R175H. Expectedly, the lines expressing Structural mp53 (R175 and R249) had lower IC50 of ATO treatment (ranging between 0.1-1 μg/ml) than those expressing wtp53 or null/truncated p53 (ranging between 0.5-10 μg/ml) (FIG. 45). In control group, Nutlin (a MDM2 inhibitor and thus a wtp53 reactivator), preferably targeted wtp53 in the cell lines we tested (FIG. 45).

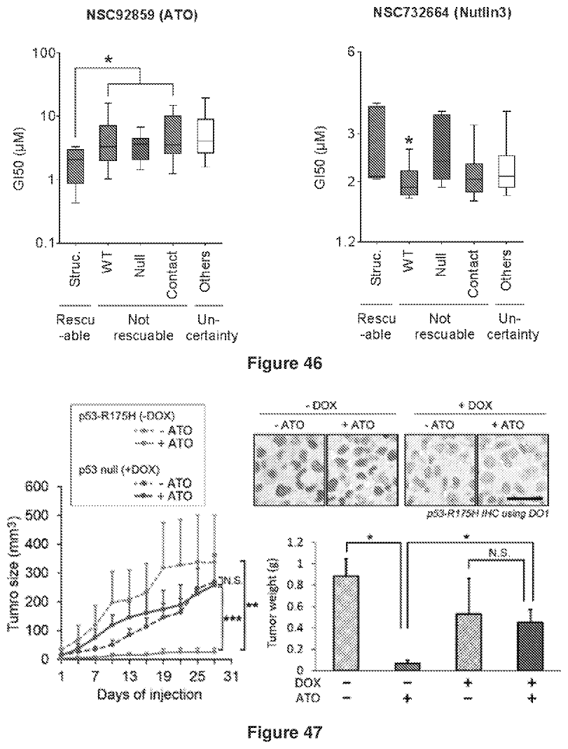

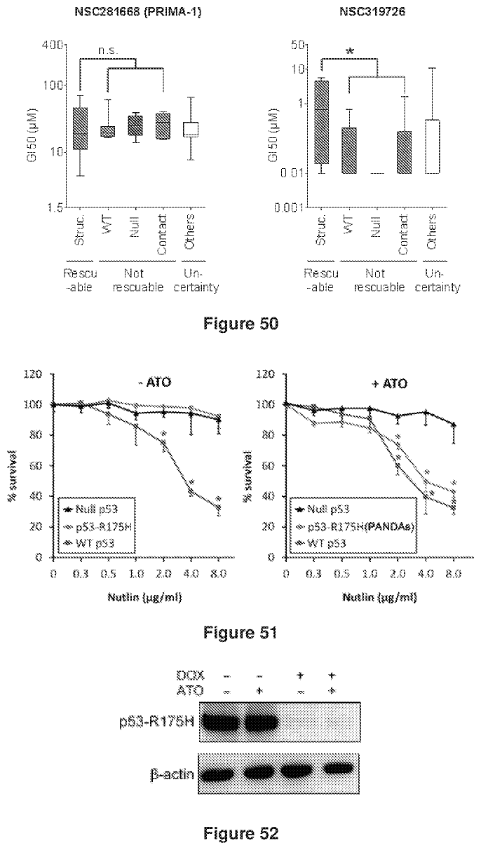

We further analyzed 60 cell lines of the NCI60 drug screen project (Shoemaker, 2006). This independently performed NCI60 screen project supplies an unbiased cell line sensitivity profile, reflecting the association between compounds and genetic features of cell lines. We separated cell lines expressing ATO-rescuable mp53 (R175, G245, R249, and R282) and designated them as “Struc”. We also separated cell lines expressing wtp53 or null/splicing p53 that was not able to be rescued by ATO (designating these as “WT” and “Null”, respectively). We then pooled the remaining cell lines were pooled together due to uncertainty regarding their rescue potential and designated them as “Others”. We found that NSC92859 (ATO) selectively inhibited the cell lines harbouring structural mp53 by exhibiting a lower G150 (concentration causing 50% growth inhibition) (Shoemaker, 2006) (FIG. 46). As expected, Nutlin selectively inhibited lines harbouring wtp53 (FIG. 46). No significant association was observed between p53 status and NSC281668 (PRIMA-1) or NSC319726 sensitivity according to this p53 classification (FIG. 50). Taken together, these results suggest that structural mp53 is a target of ATO when it inhibits malignancies.

### 5.24 PANDA Synergizes Wtp53-Reactivating Agents to Kill p53-Expressing Cells

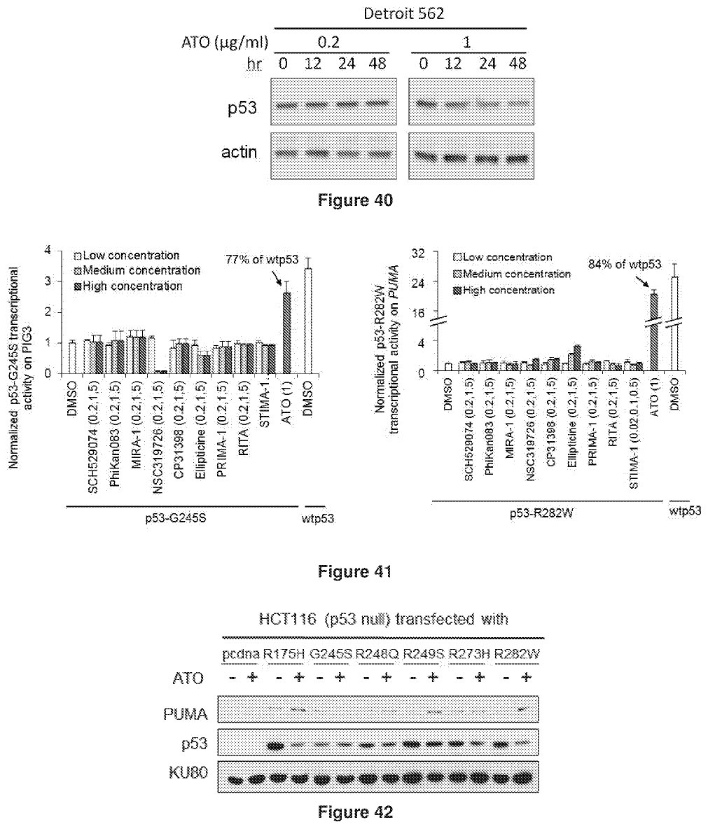

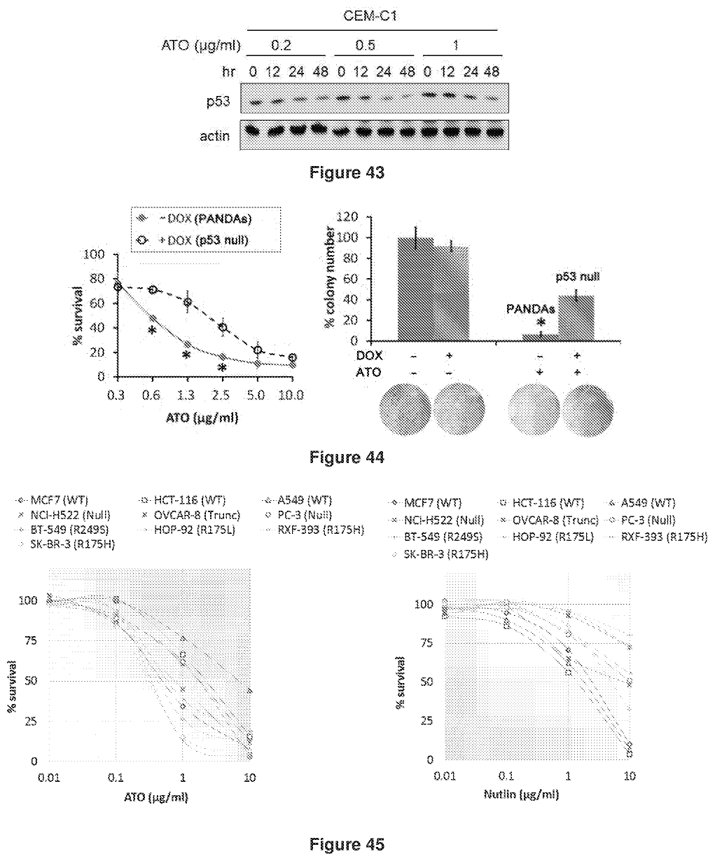

We further found that the effects of PANDA to be synergetic to the effects of wtp53-reactivating agents, such as an MDM2 inhibitor or an MDM4 inhibitor, towards killing mp53-expressing cells. The ability of a p53 rescuer and a wtp53-reactivator to work synisgetically (or at least not antagonistically) is particularly important. One reason is because one of the first targets of a rescued mp53 include its negative regulators MDM2 and MDM4. MDM2, for example is a powerful inhibitor of p53 and functions to efficiently degrade p53. In other words, when mp53 is rescued, its level also decreases. Indeed, we found p53-R175H in Detroit 562 and CEM-C1 is downregulated by ATO treatment (FIG. 40 and FIG. 43) However, in the presence of wtp53-reactivating agents, the life of rescued mp53s (PANDAs) and its tumor suppressive functions is substantially prolonged, making the ability of PANDA to work along sides MDM2 inhibitors, such as Nutlin3 extremely effective and attractive avenue for cancer therapy.

We found that the effect of the MDM2 inhibitor, Nutlin3, synergizes with the effects of ATO. (FIG. 51). For example, in the absence of ATO, 0-8 μg/ml Nutlin dose-dependently inhibits H1299 cells expressing wtp53, but not cells expressing p53-R175H or null p53 (FIG. 51). However, in the presence of ATO, Nutin is able to dose-dependently inhibit H1299 cells expressing p53-R175H.

Our finding is of significant clinical value because we showed that ATO can function in synergetic fashion with other cancer inhibition therapies, that combination anticancer therapy containing ATO has significant promises, and that ATO may increase the efficacy of the wtp53-reactivating agents, such as MDM2 inhibitors, many of which are currently under clinical trials.

### 5.25 PANDA is a Tumor Suppressor In Vivo

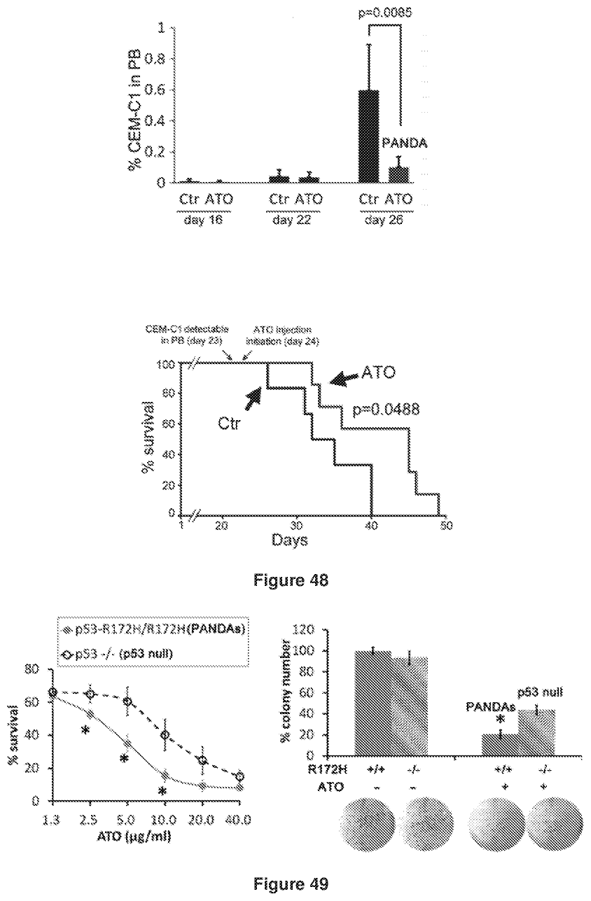

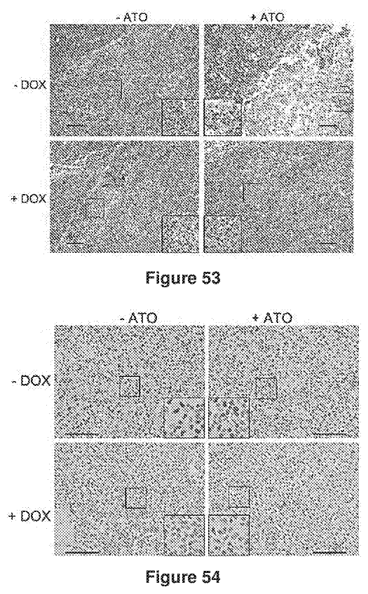

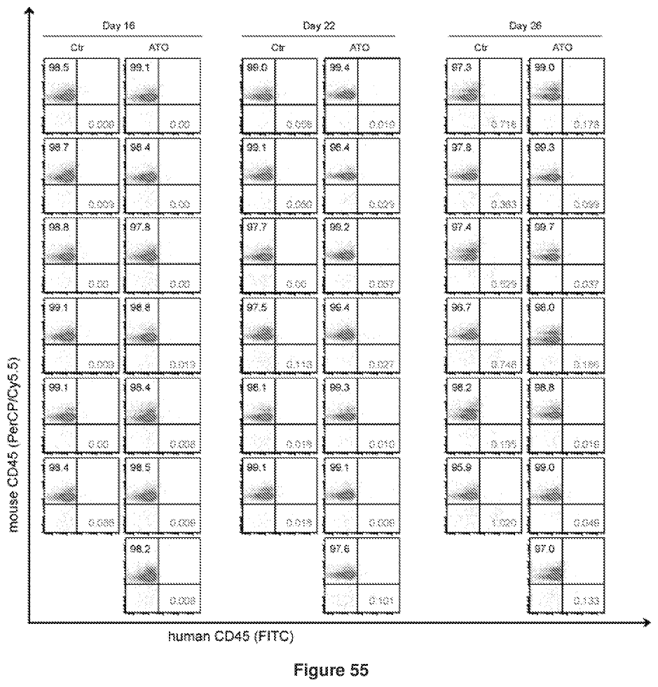

We further discovered, for the first time, that PANDAs, such as PANDA-R175H, also regains wtp53 tumor suppressive abilities in vivo, including in xenograft models. For example, we discovered that ATO and PANDA suppresses tumors in vivo, in at least two xenograft models: the H1299 cells expressing tet-off-regulated p53-R175H (solid tumor) (FIG. 47, and FIG. 52-FIG. 54) and the hematological CEM-C1 cells expressing p53-R175H (hematological malignance) (FIG. 48 and FIG. 55). For the H1299 system, we engineer the H1299 cells so that mp53 can be depleted by adding doxycycline (“DOX”).

Using the H1299 system, we injected H1299 cells subcutaneously to mouse treated with and without 5 mg/kg of ATO. We discovered that at day 28, the tumors were suppressed by over 90% according to both tumor size and tumor weight. (See FIG. 47 and FIG. 52-FIG. 54) Furthermore, we discovered that tumor suppression was predominantly PANDA-R175H-dependent, because depletion of p53-R175H by doxycycline largely abrogated the ATO and PANDA mediated tumor suppression (See FIG. 47 compare black solid line to black dot line for tumor size; compare last two bars for tumor weight).

Using the hematological CEM-C1 system, we xenografted CEM-C1 cells to mouse on day 1 by intravenously injecting the cells. We were able to detect the xenographed CEM-C1 cancer cells in the mouse peripheral blood (“PB”) on day 22 (See FIG. 48 and FIG. 55). However, administering 5 mg/kg of ATO from day 24 onwards at 6 consecutive days per week. We found the addition of ATO significantly slowed down the propagation of CEM-C1 cells in PB on day 26 (FIG. 48 and FIG. 55). We further found that the addition of ATO extended the survival of the injected mice (FIG. 48).

Taken together, we demonstrated here that ATO and PANDA significantly suppresses solid tumor and hematocancer in vivo and extends the life of subjects.

### 5.26 Combining ATO and Clinical Using Agents is Effective in Treating Cancer

To study the combination therapeutic effect of ATO, we studied the effect of widely used DNA-damaging agents in the presence or absence of ATO.

mp53 is associated with considerably poor overall survival and prognosis of a wide range of cancers, including myeloid leukemia (AML/MDS) patients (Cancer Genome Atlas Research et al., 2013; Lindsley et al., 2017). Under NCCN guidelines, the majority of recommended AMLMDS treatments, aside from APL, are DNA-damaging agents. These DNA-damaging agents are known to activate wtp53 function to kill cancer cells through p53 post-translational modifications (“PTM”s) (Murray-Zmijewski et al., 2008). These PTMs include, for example, phosphorylation, acetylation, sumoylation, neddylation, methylation, and ubiquitylation.

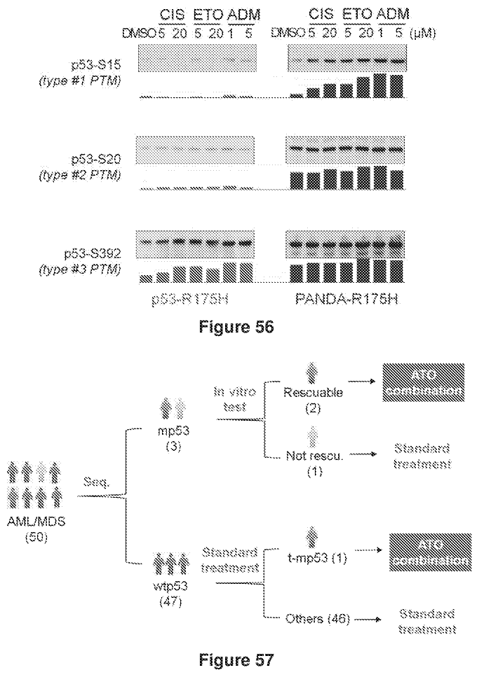

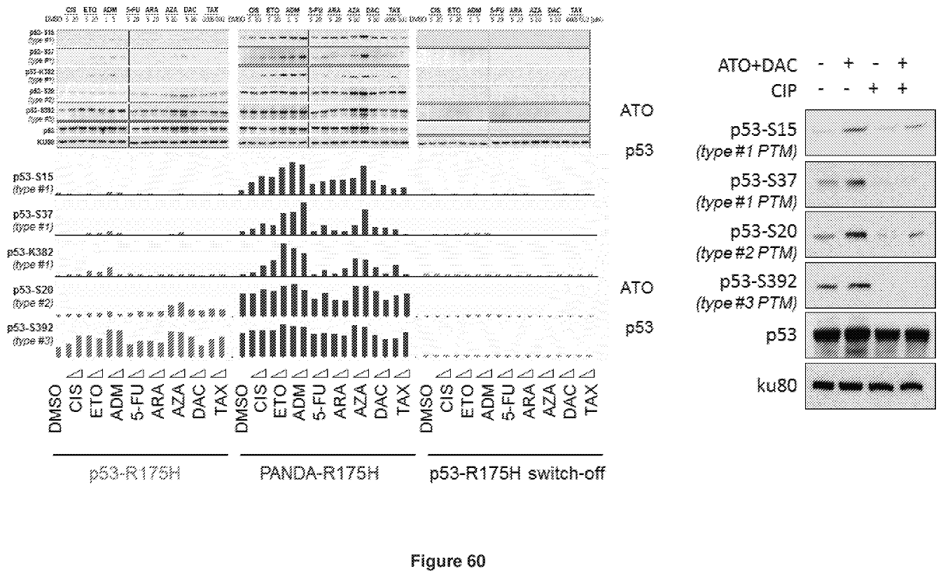

Notably, we discovered that mp53 (for example, p53-R175H) and PANDA (for example, PANDA-R175H) responded differently to the DNA-damaging agents, such as Cisplatin, Etoposide, Adriamycin/Doxorubicin, 5-Fluorouracil, Cytarabine, Azacitidine, Decitabine, and Paclitaxel, suggesting they may trigger distinctly treatment outcomes. We discovered Ser15, Ser37, and Lys382 were inertly modified on p53-R175H upon DNA-damaging treatment; however, they are actively modified on PANDA-R175H upon DNA-damaging treatment (we designated such PTM as type #1 PTM) (FIG. 56 and FIG. 60). We discovered Ser20 was inertly modified on p53-R175H irrespective of DNA-damaging stress; however it is actively modified on PANDA-R175H irrespective of DNA-damaging stress (designated as type #2 PTM). We discovered Ser392 was actively modified on both p53-R175H and PANDA-R175H even without DNA-damaging stress (designated as type #3 PTM).

The identification of type #1 PTM and type #2 PTM suggests p53-R175H and PANDA-R175H distinctly respond to therapies and thus may trigger distinctly treatment outcomes (FIG. 56 and FIG. 60). The specificity of our antibodies to phosphorylation was confirmed in for example, FIG. 60.

In addition to showing that combination therapy of ATO and DNA-damaging agents can stimulate mp53 PTM and thus reactivate mp53, we showed that the PTM differences between p53-R175H and structurally rescued PANDA-R175H supports the previously notion that Contacting mp53 (also for wtp53) differed from Structural mp53 in phosphorylation potential under DNA-damaging stress (Gillotin et al., 2010).

### 5.27 ATO and PANDA are Effective in Treating AM/MDAS Patients and Therapy can be Further Enhanced by Patient Screening

We further discovered that ATO and PANDA are effective in treating AMLMDS patients.

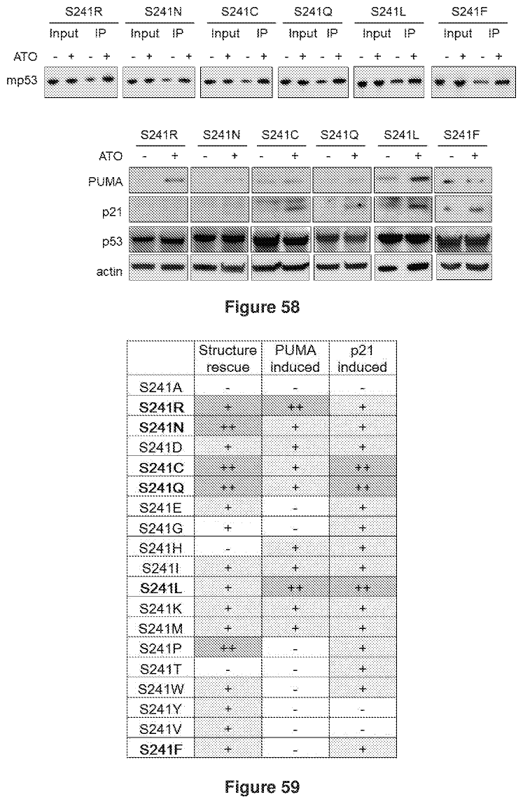

For example, we tested the therapeutic effect of treating AML/MDS patients with a combination of ATO and DNA-damaging agents. In one of our clinical trials, 50 AML/MDS patients were recruited for TP53 exome sequencing (FIG. 57). Of these, three patients were found to harbor p53 mutation (mp53 variant allele fraction >10%). In particular, we identified two patients to harbor a p53 mutation on a same residue: Patient S241F expressed p53-S241F and Patient S214C expressed p53-S241C. (FIG. 58). We further discovered that both p53-S241C and p53-S241F from the two patients behaved like Structural mp53 and reacted poorly to PAb1620 in our IP assay. (FIG. 58). However, when treated with ATO, the resulting PANDA can rescue the structure of both p53-S241C and p53-S241F. (FIG. 58). Furthermore, we discovered ATO and PANDA also significantly rescued the transcriptional activity of both p53-S241C and p53-S241F, by inducing p21, a p53 target that is responsible for cell cycle arrest, cell senescence and tumor suppression. (FIG. 58).

We also discovered a third patient, R273L, which expressed p53-R273L and found that this mp53 behaves like a wtp53 and its PAb1620 epitope cannot be further enhanced by ATO at both 4° C. and physiological temperature (37° C.) (FIG. 62).

Focusing on S241, we substituted all possible amino acids into this position and discovered that p53-S241R/N/C/Q/LF were ATO rescuable as demonstrated from their properly folded PAb1620 epitope as well as PUMA and p21 inducing ability (FIG. 58).

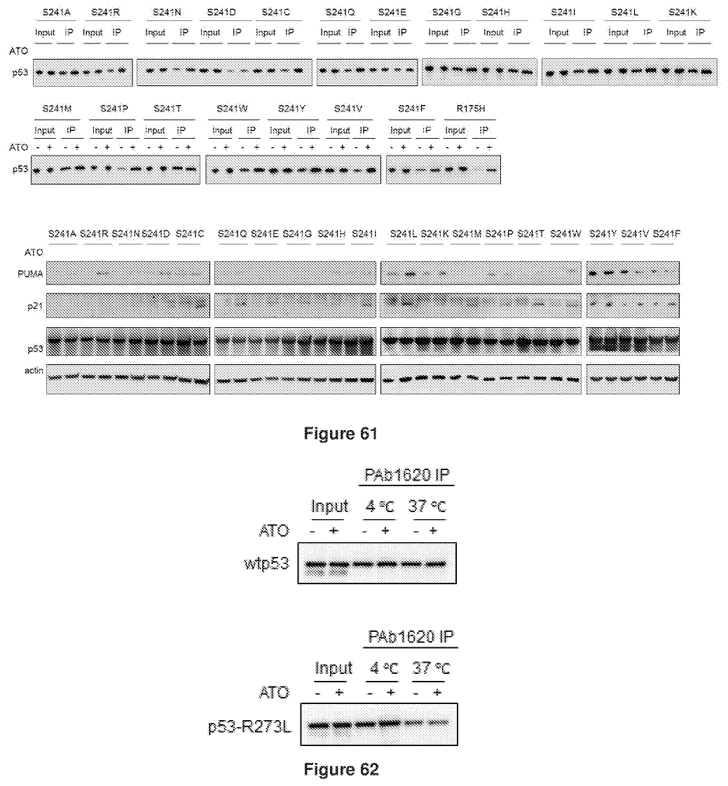

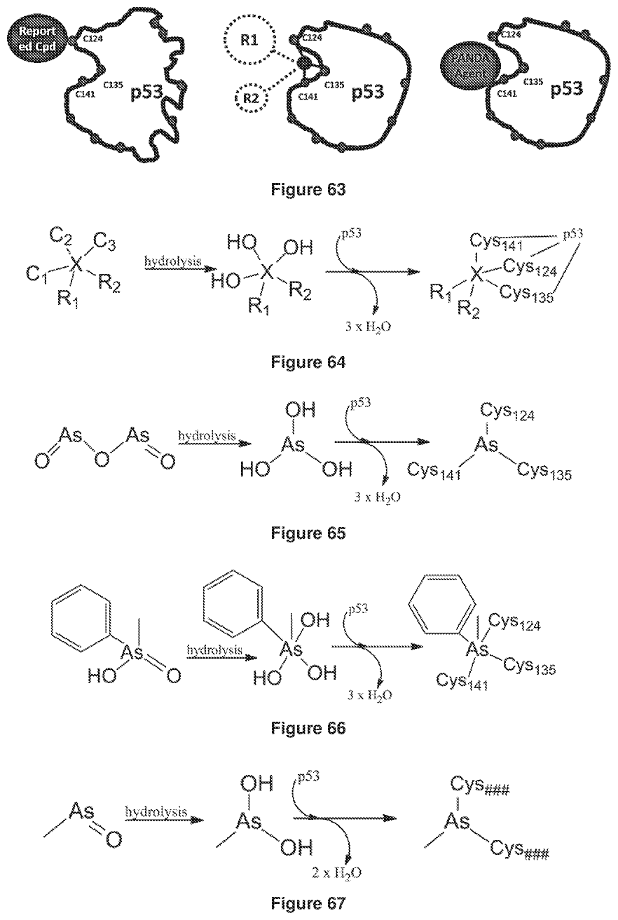

The resultant p53-S241A is not an obvious structural mp53 and thus it fails to be rescued by ATO (FIG. 59 and FIG. 61). Interestingly, p53-S241D, an obvious Structural mp53, cannot be rescued by ATO (FIG. 59 and FIG. 61). The summarized results were shown in (FIG. 59 and FIG. 61).

To further extend the finding, we tested at least 35 AML/MDS-derived mp53s in vitro and discovered that ATO can rescue the structure of these mp53 with a diversity of efficiency.

We thus selected the two ATO-rescuable MDS patients expressing p53-S241F and p53-S241C (but not the patient expressing p53-R274L) in the trial to test the combination therapeutic effects of ATO and a cytidine analog used as a first-line drug in MDS patients, such as Decitabine (“DAC”, a compound that binds to DNA and damages and also demethylates DNA), and discovered a remarkable, complete remission in both patients. Compared with standard first-line DAC regimen, we discovered mp53 expressing patients to benefit more from a combination regimen of ATO and clinically using drugs, such as DAC, as judged from their extended relapse-free survival time to about 11 months. Taken together, we have confirmed that ATO and PANDA are effective in treating cancer patients, such as AML/MDS patients, particularly those harboring PANDA-rescuable mp53s. We further discovered that treatment can be enhanced by first sequencing p53 status and then selecting patients with mp53 mutations on residues most responsive to ATO, such as mutations on S241C and S241F.

### 6. EXAMPLES

### 6.1 Plasmids, Antibodies, Cell Lines, Compounds, and Mice

pcDNA3.1 expressing human full length p53 was gift from Prof. Xin Lu (the University of Oxford), pGEX-2TK expressing fusion protein of GST and human full length p53 was purchased from Addgene (#24860), pET28a expressing p53 core was cloned for crystallization experiment without introducing any tag.

Primary antibodies were purchased from the following companies: D01 (ab1101, Abcam), PAb1620 (MABE339, EMD Millipore), PAb240 (OP29, EMD Millipore), PAb246 (sc-100, Santa Cruz), PUMA (4976, Cell signaling), PIG3 (ab96819, Abcam), BAX (sc-493, Santa Cruz), p21 (sc-817, Santa Cruz), MDM2 (OP46-100UG, EMD Millipore), Biotin (ab19221, Abcam), Tubulin (ab11308, Abcam), β-actin (A00702, Genscript), p53-S15 (9284, Cell signaling), p53-S20 (9287, Cell signaling), p53-S37 (9289, Cell signaling), p53-S392 (9281, Cell signaling), p53-K382 (ab75754, Abcam), KU80 (2753, Cell signaling). CM5 antibody was gift from Prof. Xin Lu. HRP conjugated secondary antibody specifically reacts with light chain was from Abcam (ab99632).

H1299 and Saos-2 cell lines expressing null p53 was gift from Prof. Xin Lu. H1299 cell lines expressing tet-off regulated p53-R175H or tet-on regulated wtp53 were prepared as reported previously (Fogal et al., 2005). MEFs were prepared from E13.5 TP53−/− and TP53-R172H/R172H embryos. The other cell lines were obtained from ATCC.

Compounds were purchased from the following companies: DMSO (D2650, sigma), CP31398 (PZ0115, sigma), Arsenic trioxide (202673, sigma), STIMA-1 (506168, Merck Biosciences), SCH 529074 (4240, Tocris Bioscience), PhiKan 083 (4326, Tocris Bioscience), MiRA-1 (3362, Tocris Bioscience), Ellipticine (3357, Tocris Bioscience), NSC 319726 (S7149, selleck), PRIMA-1 (S7723, selleck), RITA (NSC 652287, S2781, selleck), Cydoheximide (C7698, sigma), Biotin (A600078, Sangon Biotech), Doxycycline hydate (D9891, sigma), Cisplatin (CIS, P4394, sigma), Etoposide (ETO, E1383, sigma), Adriamycin (ADM, S1208, selleck), 5-Fluorouracil (5-FU, F6627, sigma), Cytarabine (ARA, S1648, selleck), Azacitidine (AZA, A2385, sigma), Decitabine (DAC, A3656, sigma), Paclitaxel (TAX, S1150, selleck). Bio-As and Bio-Dithi-As were gift from Kenneth L. Kirk (NIH; PMID: 18396406).

The TP53 wild-type mice, female nude mice and NOD/SCID mice were obtained from the Shanghai Laboratory Animal Center, Chinese Academy of Sciences. TP53-R172H/R172H mice were generated from the parent mice (026283) purchased from Jackson Lab. TP53−/− mice (002101) were purchased from National Resource Center of Model Mice of China.

DNA samples were sequenced in rainbow-genome technique Ltd (Shanghai) and Shanghai Biotechnology corporation (Shanghai).

### 6.2 Preparation of PANDA (without p53's N-Terminus and C-Terminus, without Tag) Formed in Bacteria

Constructions expressing recombinant p53 core were transformed into E. coli strain BL21-Gold. Cells were cultured in either LB or M9 medium at 37° C. to mid-log phase. 0.5 mM isopropyl-β-D-thiogalactopyranoside (IPTG) was added in presence/absence of 50 μM As/Sb/Bi and 1 mM ZnCl₂ at 25° C. for overnight. Cells were harvested by centrifugation at 4 000 RPM for 20 minutes (˜10 g cell paste yielded from 1 liter of medium) and then sonicated in lysate buffer (50 mM Tris, pH 7.0, 50 mM NaCl, 10 mM DTT and 1 mM phenylmethylsulfonyl fluoride) in presence/absence of 50 μM As/SbBi. Soluble lysate was loaded onto a SP-Sepharose cation exchange column (Pharmacia) and eluted with a NaCl gradient (0-1 M) then, if necessary, additionally purified by affinity chromatography with a heparin-Sepharose column (Pharmacia) in Tris.HCl, pH 7.0, 10 mM DTT with a NaCl gradient (0-1 M) for elution. Future purification was performed by gel-filtration using Superdex 75 column using standard procedure.

Processes after cell lysing are done at 4° C. Protein concentration was measured spectrophotometrically by using an extinction coefficient of 16 530 cm⁻¹M⁻¹ at 280 nm. All protein purification steps were monitored by 4-20% gradient SDS-PAGE to ensure they were virtually homogeneous.

### 6.3 Preparation of PANDA (with GST Tag) Formed in Bacteria

Constructions expressing GST-p53 (or GST-mp53) were transformed into E. coli strain BL21-Gold. Cells were grown in 800 ml LB medium at 37° C. to mid-log phase. 0.3 mM IPTG with/without 50 μM As/Sb/Bi was added at 16° C. for 24 h. Cells were harvested by centrifugation at 4 000 RPM for 20 minutes and then sonicated in 30 ml lysate buffer (58 mM Na2HPO4.12H2O, 17 mM NaH2 PO4.12H2O, 68 mM NaCl, 1% Triton X-100) in presence/absence of 50 μM As/Sb/Bi. Cell supernatant after 9000 RMP for 1 hour was added with 400 μl glutathione beads (Pharmacia) and incubated overnight. Beads were washed with lysate buffer for 3 times. Recombinant protein was then eluted by 300 μl elution buffer (10 mM GSH, 100 mM NaCl, 5 mM DTT and 50 mM Tris-HCl, pH 8.0). Processes after cell lysing are done at 4° C. All protein purification steps were monitored by 4-20% gradient SDS-PAGE to ensure they were virtually homogeneous.

### 6.4 Preparation of PANDA Formed in Insect Cells

Baculovirus infected Sf9 cells expressing recombinant human full-length p53 or p53 core in presence/absence of 50 μM As/Sb/Bi were harvested. They lysed in lysate buffer (50 mM Tris-HCl, pH 7.5, 5 mM EDTA, 1% NP-40, 5 mM DTT, 1 mM PMSF, and 0.15 M NaCl) in presence/absence of 50 μM As/SbBi. The lysates were then incubated on ice for 30 min, followed by centrifuging at 13000 rpm for 30 min. The supernatant was diluted 4-fold using 15% glycerol, 25 mM HEPES, pH 7.6, 0.1% Triton X-100, 5 mM DTT and 1 mM Benzamidine. They were further filtered using a 0.45 mm filter, and purified by Heparin-Sepharose column (Pharmacia). Purified protein was then concentrated using YM30 Centricon (EMD, Millipore). All protein purification steps were monitored by 4-20% gradient SDS-PAGE to ensure they were virtually homogeneous.

### 6.5 Preparation of PANDA Formed In Vitro

PANDA can be efficiently formed by mixing p53, either purified p53 or p53 in cell lysate, with PANDA Agents. For example, in reaction buffer (20 mM HEPES, 150 mM NaCl, pH 7.5), we mixed purified recombinant p53 core and As/Sb/Bi compounds in a ratio ranging from 10:1-1:100 at 4° C. for overnight. The formed PANDA was then purified using dialysis to eliminate compounds.

### 6.6 In Vitro Reaction of Recombinant GST-p53-R175H and as

50 μM purified recombinant protein GST-p53-R175H in reaction buffer (10 mM GSH, 100 mM NaCl, 5 mM DTT and 50 mM Tris-HCl, pH 8.0) was added with Biotin-As to obtain arsenic to p53 molar ratio of either 10:1 or 1:1. The mixture solution was incubated at 4° C. for overnight and then divided into three parts. Each part was subjected to SDS-PAGE, followed by Coomassie blue staining (5 μg GST-p53-R175H applied), p53 immunoblotting (0.9 μg GST-p53-R175H applied) or Biotin immunoblotting (5 μg GST-p53-R175H applied), respectively.

### 6.7 Immunoprecipitation

For immunoprecipitation, mammalian cells or bacteria cells were harvested and lysed in NP40 buffer (50 mM Tris-HCl pH 8.0, 150 mM NaCl, 1% NP40) with cocktail of protease inhibitors (Roche Diagnostics). Cell lysates were then sonicated for 3 times, followed by spinning at 13,000 RPM for 20 min. Supernatant was adjusted to a final concentration of 1 mg/ml total protein using 450 μl NP40 buffer and incubated with 20 μl protein G beads and 1-3 μg corresponding primary antibody for 2 hr at 4° C. The beads were washed for three times with 20-25° C. NP40 buffer at room temperature. After spinning down, the beads were boiled for 5 min in 2×SDS loading buffer, followed by Western blotting.

### 6.8 Biotin-Arsenic Based Pull-Down Assay

Cells were treated with 4 μg/ml Bio-As or Bio-dithi-As for 2 hours. Cells were lysed in NP40 buffer (50 mM Tris-HCl pH 8.0, 150 mM NaCl, 1% NP40) with cocktail of protease inhibitors (Roche Diagnostics). Cell lysates were then sonicated for 3 times, followed by spinning at 13,000 RPM for 1 hr. Supernatant was adjusted to a final concentration of 1 mg/ml total protein using 450 μl NP40 buffer and incubated with 20 μl streptavidin beads for 2 hr at 4° C., followed by bead washing and Western blotting.

### 6.9 Biotin-DNA Based Pull-Down Assay

To prepare double-stranded oligonucleotides, equal amount of complementary single stranded oligonucleotides were heated at 80° C. for 5 min in 0.25 M NaCl, followed by slow cooling to room temperature. Sequences of single stranded oligonucleotides were followed:

Consensus
5′-Biotin-TCGAGAGGCATGTCTAGGCATGTCTC
PUMA
5′-Biotin-CTGCAAGTCCTGACTTGTCC
PIG3
5′-Biotin-AGAGCCAGCTTGCCCACCCATGCTCGCGTG
BAX
5′-Biotin-TCACAAGTTAAGACAAGCCTGGGCGTGGGC
MDM2
5′-Biotin-CGGAACGTGTCTGAACTTGACCAGCTC
p21
5′-Biotin-CGAGGAACATCTCCCAACATGTTTGCTGAG
Consensus-R
5′-GAGACATGCCTAGACATGCCTCTCGA
PUMA-R
5′-GGACAAGTCAGGACTTGCAG
PIG3-R
5′-CACGCGAGCATGGGTGGGCAAGCTGGCTCT
BAX-R
5′-GCCCACGCCCAGGCTTGTCTTAACTTGTGA
MDM2-R
5′-GAGCTGGTCAAGTTCAGACACGTTCCG
p21-R
5′-CTCGAGCAACATGTTGGGACATGTTCCTCG

Cells were harvested and lysed in NP40 buffer (50 mM Tris-HCl pH 8.0, 150 mM NaCl, 1% NP40) with cocktail of protease inhibitors (Roche Diagnostics). Cell lysates were then sonicated for 3 times, followed by spinning at 13,000 RPM for 1 hr. Supernatant was adjusted to a final concentration of 1 mg/ml total protein using 450 μl NP40 buffer and incubated with 20 μl streptavidin beads (s-951, Invitrogen), 20 μmoles of biotinylated double-stranded oligonucleotides, and 2 μg of poly(dI-dC) (sc-286691, Santaz cruz). Lysates were incubated for 2 hr at 4° C., followed by bead washing and immunoblotting.

### 6.10 Immunoblotting

Immunoblotting was performed as reported previously (Lu et al., 2013).

### 6.11 Luciferase Assay

Cells were plated at a concentration of 2×10⁴ cells/well in 24-well plates, followed by transfection of luciferase reporter plasmids for 24 hr. All transfection contained 300 ng p53 expressing plasmid, 100 ng of luciferase reporter plasmid and 5 ng of Renilla plasmid per well. After agent treatment, cells were lysed in luciferase reporter assay buffer and determined using a luciferase assay kit (Promega). Activities of luciferase were divided by that of Renilla to normalize the transfection efficiency. For more details, see (Lu et al., 2013).

### 6.12 Colony Formation Assay

Treated cells were digested with trypsin. 100, 1000 or 10,000 cells/well were seeded in 12-well plates and kept in culture for 2-3 weeks. Fresh medium was replaced every three days.

### 6.13 Non-Denaturing PAGE

Cells were lysed in either CHAPS buffer (18 mM 3-[(3-cholamidopropyl) dimethylammonio]-1-propanesulfonic acid in TBS) or M-PER buffer (78501, Invitrogen) containing DNase and protease inhibitors for 15 min at 4° C. or 37° C. Cell lysate was added with 20% glycerol and 5 mM Coomassie G-250 before loading into 3-12% Novex Bis-Tris gradient gels. The electrophoresis was performed at 4° C. according to the manufacturer's instructions. Proteins were transferred onto the polyvinylidene fluoride membranes and fixed with 8% acetic acid for 20 min. The fixed membranes were then air dried and destained with 100% methanol. Membranes were blocked for overnight with 4% BSA in TBS at 4° C. before immunoblotting.

### 6.14 Real Time qPCR

Total RNA was isolated from cells using Total RNA Purification Kit (B518651, Sangon Biotech). 1 μg total RNA was reverse-transcribed using the GoScript®™ Reverse Transciptase System (A5001, Promega) following manufacturer's protocol. PCR was performed in triplicate using SYBR green mix (Applied Biosystems), and a ViiA™ 7 Real-Time PCR System (Applied Biosystems) under the following conditions: 10 min at 95° C. followed by 40 cycles of 95° C. for 15 s and 60° C. for 1 min. Specificity of the PCR product was checked for each primer set and samples from the melting curve analysis. Expression levels of targeted genes were normalized relative to levels of β-actin adopting comparative Ct method. The primer sequences are as follows: MDM2 forward 5′-CCAGGGCAGCTACGGTTTC-3′, reverse 5′-CTCCGTCATGTGCTGTGACTG-3′; PIG3 forward 5′-CGCTGAAATTCACCAAAGGTG-3′, reverse 5′-AACCCATCGACCATCAAGAG-3′; PUMA forward 5′-ACGACCTCAACGCACAGTACG-3′, reverse 5′-TCCCATGATGAGATTGTACAGGAC-3′; p21 forward 5′-GTCTTGTACCCTTGTGCCTC-3′, reverse 5′-GGTAGAAATCTGTCATGCTGG-3′; Bax forward 5′-GATGCGTCCACCAAGAAGCT-3′, reverse 5′-CGGCCCCAGTTGAAGTTG-3′; β-actin forward 5′-ACTTAGTTGCGTTACACCCTTTCT-3′, reverse 5′-GACTGCTGTCACCTTCACCGT-3′.

### 6.15 Xenograft Assay

H1299 xenograft. H1299 cells expressing tet-off regulated p53-R175H (1*10⁶ cells) suspended in 100 μl saline solution were subcutaneously injected into the flanks of 8-9 weeks old female nude mice. When the tumor area reached 0.1 cm (day 1), 5 mg/kg ATO were intraperitoneally injected 6 consecutive days per week. In DOX groups, 0.2 mg/ml doxycycline was added to drinking water. Tumor size was measured every 3 days with vernier callipers. Tumor volumes were calculated using the following formula: (L*W*W)/2, in which L represents the large diameter of the tumor, and W represents the small diameter. When tumor area reached ˜1 cm diameter in any group, mice were sacrificed and isolated tumors were weighed. The analysis of the differences between the groups was performed by Two-way RM ANOVA with Bonferroni correction.

CEM-C1 xenograft. 8-9 week old NOD/SCID mice were intravenously injected through the tail vein with 1*10⁷ cells of CEM-C1 T-ALL cells (day 1). After engraftment, peripheral blood samples were obtained from the mice retro-orbital sinus every 3 or 4 days from day 16 to day 26. Residual red blood cells were removed using erythrocyte lysis buffer (NH₄Cl 1.5 mM, NaHCO₃ 10 Mm, EDTA-2Na 1 mM). The isolated cells were double stained with PerCP-Cy5.5-conjugated anti-mouse CD45 (mCD45) (BD Pharmigenm, San Diego, Calif.) and FITC-conjugated anti-human CD45 (hCD45) (BD Pharmigen™, San Diego, Calif.) antibodies before flow cytometric analysis conducted. When the percent of hCD45+ cells in peripheral blood reached 0.1% one mice (day 22), ATO was prepared for injection. On day 23, 5 mg/kg ATO were intravenously injected via tail-vein in 0.1 ml saline solution 6 consecutive days per week. The comparison of the hCD45+ cells percent between the groups was performed by unpaired t test. The life-span of mice was analyzed by Log-rank (Mantel-Cox) test.

All statistical analysis was performed using GraphPad Prism 6.00 for Windows (La Jolla Calif., USA). The animals were housed in specific pathogen-free conditions. Experiments were carried out according to the National Institutes of Health Guide for Care and Use of Laboratory Animals.

### 6.16 ATO Greatly Increases Mp53 Stability by Increasing its Melting Temperature

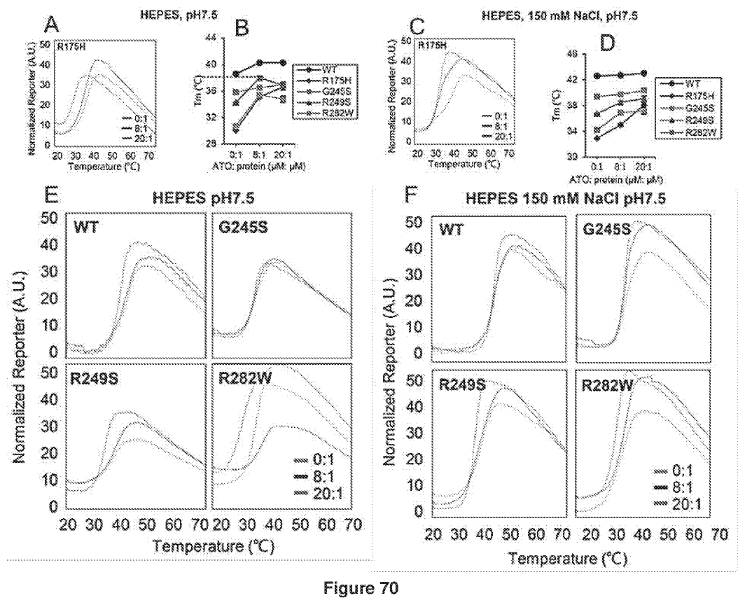

We measured the melting curve of the purified p53 core domain R175H(94-293) recorded via differential scanning fluorimetry (DSF) at the indicated ratio of ATO in pH 7.5 HEPES buffer. (See FIG. 70 A).

We further mixed ATO and the purified recombinant p53C (p53C-WT, p53C-R175H, p53C-G245S, p53C-R249S and p53C-R282W, 5 μM for each reaction) at the ratios indicated in FIG. 70B in pH 7.5 HEPES buffer for overnight. Melting curves of the p53C were measured by DSF in pH 7.5 HEPES buffer. The apparent Tₘ of the p53C-R175H, p53C-G245S, p53C-R249S, and p53C-R282W can be raised by 1.1-6.5° C. by maximum in pH 7.5 HEPES buffer. The melting temperatures of p53 core were shown (mean±SD, n=3). (See FIG. 70B).

We further measured the melting curve of the purified p53 core domain R175H(94-293) recorded via differential scanning fluorimetry at the indicated ratio of ATO in pH 7.5 HEPES, 150 mM NaCl buffer. (See FIG. 70C).

We further mixed ATO and the purified recombinant p53C (p53C-WT, p53C-R175H, p53C-G245. p53C-R249S and p53C-R282W, 5 μM for each reaction) at the ratios in FIG. 70D in pH 7.5 HEPES, 150 mM NaCl buffer for overnight. Melting curves of the p53C were measured by DSF in pH 7.5 HEPES, 150 mM NaCl buffer. The apparent Tₘ of the p53C-R175H, p53C-G245S, p53C-R249S, and p53C-R282W can be raised by 1.0-5.1° C. by maximum in pH 7.5 HEPES, 150 mM NaCl buffer. The melting temperatures of p53 core were shown (mean±SD, n=3). (See FIG. 70D).

We further measured the melting curve of the purified p53 core domain (p53C-WT, p53C-G245S, p53C-R249S and p53C-R282W) via differential scanning fluorimetry at the indicated ratio of ATO in pH 7.5 HEPES buffer. (See FIG. 70E).

We further measured the melting curve of the purified p53 core domain (p53C-WT, p53C-G245S, p53C-R249S and p53C-R282W) via differential scanning fluorimetry at the indicated ratio of ATO in pH 7.5 HEPES, 150 mM NaCl buffer. (See FIG. 70F).

Together, our results showed that the melting temperature of the p53 incubated with ATO was recorded via differential scanning fluorimetry. The Tₘ of p53 incubated was raised in pH 7.5 HEPES buffer in the presence or absence of 150 mM NaCl. In HEPES buffer, Tm of the p53C-R175H, p53C-G245S, p53C-R249S, and p53C-R282W can be raised by for example, 6.5° C., 1.1° C., 3.7° C., and 4.7° C. respectively (FIG. 70 B). In HEPES, 150 mM NaCl buffer, Tₘ of the p53C-R175H, p53C-G245S, p53C-R249S, and p53C-R282W can be raised by for example, 5.1° C., 1.0° C., 2.3° C., and 3.0° C. respectively (FIG. 70 D). The data indicated that p53C-WT can also be stabilized slightly. The peak curve of the represented p53C-R175H in FIGS. 70A and 70C was shifted to right incubated with ATO showed PANDA-R175H was more stable than p53C-R175H under the same temperature. Similar data was recorded in the p53C-WT, p53C-G245S, p53C-R249S and p53C-R282W. (See FIGS. 70E and 70F).

### 6.17 PANDA Regains Transcriptional Activities on Most of the p53 Target Genes

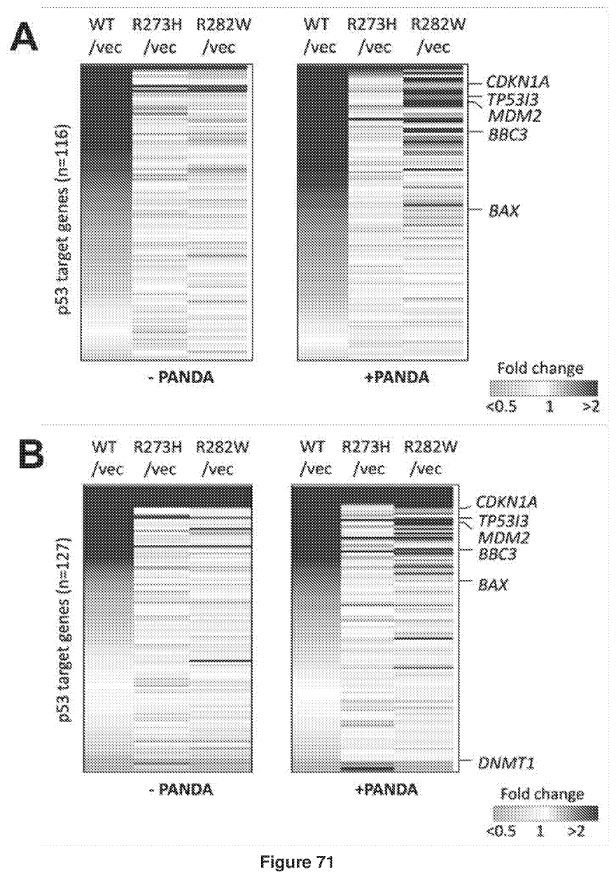

We transfected SaOS-2 cells with wtp53, p53-R273H or p53-R282W and were treated with 1 μg/ml ATO for 24 hr. Expression levels of the p53 targets were determined by RNA-sequencing. The heatmap of the fold change values (the indicated sample groups versus vector) of the reported 116 p53-activated targets were measured. (See FIG. 71A). The heatmap of the fold change values of a set of 127 p53 targets identified in another research were also measured. (See FIG. 71B).

We further determined the function of formed PANDA among p53 targets using RNA sequencing (RNA-seq). It was found that, among the reported 116 genes p53-activated targets, the majority of the genes were up-regulated by PANDA-R282W, including the well-known p53 targets BBC3, BAX, TP5313, CDKN1A, and MDM2 (FIG. 71A). Similar results were also found in the 127 p53 targets (including p53-activated and -repressed genes) identified in another research (FIG. 71B). By comparison, it was not obvious that the transcriptional activity for PANDA-R273H, which involved a contacting mutant p53, was not restored at the similar levels.

### 6.18 Statistical Analysis

Statistical analysis was carried out using Fisher's exact test (two-tailed) unless otherwise indicated. p values less than 0.05 were considered statistically significant unless otherwise indicated.

TABLE 1
1100 three valence arsenic (“As”) containing compounds were predicted to efficiently bind PANDA Pocket
and efficiently rescue structural mp53. All of the 94.2 million structures recorded in PubChem
(https://pubchem.ncbi.nlm.nih.gov/) were applied for 4C+ screening. In the 4C+ screening, we collected those
with more than 2 cysteine-binding potential. Carbon-binding As/Sb/Bi bond has defect in binding cysteine since this
bond cannot be hydrolyzed. The other As/Sb/Bi bond can be hydrolyzed in cells and thus is able to bind cysteine.
Arsenic 3 Valence PANDA Agents

| | | | | | |
|---|---|---|---|---|---|
| CID 544 | CID 545 | CID 14,770 | CID 14,772 | CID 19,167 | CID 19,541 |
| CID 21,763 | CID 23,969 | CID 24,569 | CID 24,570 | CID 24,571 | CID 24,575 |
| CID 24,577 | CID 24,679 | CID 24,967 | CID 25,130 | CID 25,156 | CID 25,214 |
| CID 26,435 | CID 33,719 | CID 40,516 | CID 48,705 | CID 61,545 | CID 61,788 |
| CID 62,222 | CID 62,391 | CID 76,591 | CID 78,450 | CID 82,779 | CID 82,780 |
| CID 82,781 | CID 82,782 | CID 82,783 | CID 82,784 | CID 82,785 | CID 82,786 |
| CID 82,787 | CID 82,876 | CID 82,877 | CID 82,878 | CID 82,879 | CID 82,880 |
| CID 82,998 | CID 82,999 | CID 83,000 | CID 83,001 | CID 83,002 | CID 83,003 |
| CID 84,670 | CID 89,859 | CID 91,500 | CID 96,378 | CID 109,033 | CID 116,252 |
| CID 116,256 | CID 116,257 | CID 117,908 | CID 122,957 | CID 138,780 | CID 139,279 |
| CID 139,298 | CID 139,321 | CID 139,322 | CID 139,323 | CID 139,324 | CID 139,773 |
| CID 139,891 | CID 139,939 | CID 140,012 | CID 143,005 | CID 145,013 | CID 150,167 |
| CID 158,274 | CID 159,390 | CID 165,514 | CID 167,244 | CID 167,245 | CID 167,445 |
| CID 178,411 | CID 178,412 | CID 178,413 | CID 178,414 | CID 178,415 | CID 178,416 |
| CID 180,508 | CID 184,133 | CIC 187,781 | CID 193,333 | CID 197,119 | CID 204,633 |
| CID 206,883 | CID 211,075 | CID 227,557 | CID 261,004 | CID 284,218 | CID 291,833 |
| CID 305,699 | CID 314,295 | CID 410,123 | CID 443,495 | CID 444,994 | CID 469,401 |
| CID 518,605 | CID 518,740 | CID 534,808 | CID 575,145 | CID 597,659 | CID 598,023 |
| CID 602,148 | CID 606,254 | CID 607,452 | CID 635,386 | CID 2,724,938 | CID 2,784,590 |
| CID 3,019,767 | CID 3,019,768 | CID 3,033,858 | CID 3,051,241 | CID 3,051,242 | CID 3,051,637 |
| CID 3,051,638 | CID 3,055,940 | CID 3,083,023 | CID 3,627,253 | CID 4,093,503 | CID 4,390,915 |
| CID 4,628,691 | CID 5,127,350 | CID 5,148,939 | CID 5,149,849 | CID 5,231,567 | CID 5,245,648 |
| CID 5,246,853 | CID 5,247,035 | CID 5,247,036 | CID 5,247,256 | CID 5,247,881 | CID 5,248,768 |
| CID 5,248,769 | CID 5,255,179 | CID 5,256,195 | CID 5,256,504 | CID 5,258,395 | CID 5,258,981 |
| CID 5,259,398 | CID 5,259,672 | CID 5,460,506 | CID 5,460,562 | CID 5,460,564 | CID 5,460,565 |
| CID 5,460,566 | CID 5,460,587 | CID 5,460,722 | CID 5,491,620 | CID 5,743,819 | CID 5,743,820 |
| CID 6,284,547 | CID 6,326,784 | CID 6,327,292 | CID 6,329,460 | CID 6,329,630 | CID 6,329,663 |
| CID 6,333,458 | CID 6,334,574 | CID 6,335,843 | CID 6,337,007 | CID 6,391,215 | CID 6,395,537 |
| CID 6,395,538 | CID 6,395,543 | CID 6,395,544 | CID 6,395,864 | CID 6,395,865 | CID 6,395,866 |
| CID 6,395,867 | CID 6,395,868 | CID 6,397,860 | CID 6,398,629 | CID 6,711,222 | CID 6,857,430 |
| CID 6,857,431 | CID 6,857,580 | CID 6,914,516 | CID 6,914,518 | CID 6,914,531 | CID 9,548,861 |
| CID 9,548,868 | CID 9,548,869 | CID 9,553,924 | CID 9,553,925 | CID 9,823,432 | CID 9,940,355 |
| CID 10,285,774 | CID 10,540,385 | CID 10,709,168 | CID 10,714,189 | CID 10,716,984 | CID 10,717,398 |
| CID 10,744,851 | CID 10,752,266 | CID 10,886,649 | CID 10,887,184 | CID 10,887,185 | CID 10,896,360 |
| CID 10,908,016 | CID 10,930,378 | CID 10,963,436 | CID 11,010,611 | CID 11,010,612 | CID 11,017,329 |
| CID 11,017,851 | CID 11,017,852 | CID 11,049,547 | CID 11,134,592 | CID 11,148,325 | CID 11,193,862 |
| CID 11,248,058 | CID 11,251,274 | CID 11,256,472 | CID 11,298,875 | CID 11,310,398 | CID 11,316,887 |
| CID 11,385,570 | CID 11,402,381 | CID 11,412,921 | CID 11,431,015 | CID 11,465,219 | CID 11,476,512 |
| CID 11,562,561 | CID 11,607,348 | CID 11,650,722 | CID 11,676,828 | CID 11,686,352 | CID 11,706,542 |
| CID 11,751,340 | CID 11,831,174 | CID 11,966,301 | CID 11,966,302 | CID 11,968,269 | CID 11,970,814 |
| CID 11,970,815 | CID 11,980,559 | CID 12,022,729 | CID 12,029,075 | CID 12,060,026 | CID 12,060,027 |
| CID 12,060,028 | CID 12,060,029 | CID 12,062,780 | CID 12,062,953 | CID 12,062,354 | CID 12,105,505 |
| CID 12,549,303 | CID 12,549,305 | CID 12,549,308 | CID 12,622,637 | CID 12,639,434 | CID 12,690,546 |
| CID 12,690,547 | CID 12,692,939 | CID 12,754,317 | CID 12,754,318 | CID 12,758,717 | CID 12,758,718 |
| CID 12,758,720 | CID 12,909,455 | CID 12,946,537 | CID 12,946,538 | CID 12,964,989 | CID 12,970,646 |
| CID 13,068,571 | CID 13,076,527 | CID 13,426,139 | CID 13,464,647 | CID 13,528,538 | CID 13,528,542 |
| CID 13,528,544 | CID 13,528,552 | CID 13,710,813 | CID 13,726,197 | CID 13,751,567 | CID 14,042,655 |
| CID 14,226,398 | CID 14,389,419 | CID 14,420,822 | CID 14,951,535 | CID 14,962,238 | CID 15,130,450 |
| CID 15,165,101 | CID 15,193,599 | CID 15,193,603 | CID 15,214,107 | CID 15,241,817 | CID 15,393,356 |
| CID 15,443,371 | CID 15,443,375 | CID 15,452,707 | CID 15,452,708 | CID 15,452,709 | CID 15,463,897 |
| CID 15,544,525 | CID 15,763,917 | CID 15,768,782 | CID 15,770,833 | CID 15,770,922 | CID 15,773,246 |
| CID 15,775,979 | CID 15,779,670 | CID 15,784,977 | CID 15,787,963 | CID 15,787,964 | CID 15,793,134 |
| CID 15,801,408 | CID 15,804,634 | CID 15,831,253 | CID 15,831,254 | CID 15,836,478 | CID 15,836,479 |
| CID 15,836,480 | CID 15,845,941 | CID 15,902,288 | CID 15,909,316 | CID 15,911,001 | CID 16,103,935 |
| CID 16,122,615 | CID 16,122,617 | CID 16,217,311 | CID 16,687,467 | CID 16,687,468 | CID 16,688,418 |
| CID 16,688,647 | CID 16,688,985 | CID 17,950,332 | CID 17,972,457 | CID 17,972,458 | CID 17,999,318 |
| CID 18,620,370 | CID 18,699,306 | CID 18,764,508 | CID 18,924,269 | CID 18,982,511 | CID 19,067,450 |
| CID 19,076,933 | CID 19,377,186 | CID 19,770,611 | CID 19,827,518 | CID 19,882,979 | CID 19,977,789 |
| CID 19,977,790 | CID 20,194,853 | CID 20,194,857 | CID 20,209,387 | CID 20,209,389 | CID 20,313,265 |
| CID 20,363,283 | CID 20,386,190 | CID 20,481,422 | CID 20,498,187 | CID 20,519,937 | CID 20,519,998 |
| CID 20,745,926 | CID 20,838,028 | CID 20,843,076 | CID 20,843,447 | CID 20,843,448 | CID 20,846,387 |
| CID 20,846,388 | CID 20,976,124 | CID 21,096,828 | CID 21,117,048 | CID 21,119,276 | CID 21,119,939 |
| CID 21,119,940 | CID 21,124,621 | CID 21,126,002 | CID 21,126,578 | CID 21,127,708 | CID 21,152,268 |
| CID 21,153,195 | CID 21,153,196 | CID 21,396,696 | CID 21,402,593 | CID 21,498,356 | CID 21,597,738 |
| CID 21,597,741 | CID 21,666,086 | CID 21,679,197 | CID 21,679,198 | CID 21,718,489 | CID 21,719,691 |
| CID 21,720,503 | CID 21,732,336 | CID 21,732,337 | CID 21,732,338 | CID 21,732,339 | CID 21,732,340 |
| CID 21,732,341 | CID 21,732,582 | CID 21,732,691 | CID 21,732,985 | CID 21,854,934 | CID 21,854,935 |
| CID 21,854,936 | CID 21,854,937 | CID 21,854,938 | CID 21,854,939 | CID 21,854,940 | CID 21,854,941 |
| CID 21,854,942 | CID 21,854,943 | CID 21,854,944 | CID 21,854,945 | CID 21,854,946 | CID 21,854,947 |
| CID 21,854,948 | CID 21,854,949 | CID 21,896,088 | CID 21,932,415 | CID 21,954,723 | CID 21,998,317 |
| CID 22,099,016 | CID 22,119,650 | CID 22,223,246 | CID 22,239,183 | CID 22,341,918 | CID 22,413,077 |
| CID 22,717,370 | CID 22,833,492 | CID 22,895,849 | CID 23,028,044 | CID 23,234,053 | CID 23,234,054 |
| CID 23,235,595 | CID 23,235,987 | CID 23,261,952 | CID 23,261,953 | CID 23,261,954 | CID 23,261,955 |
| CID 23,261,956 | CID 23,261,957 | CID 23,261,958 | CID 23,261,959 | CID 23,261,960 | CID 23,261,961 |
| CID 23,261,962 | CID 23,261,963 | CID 23,261,964 | CID 23,261,965 | CID 23,261,966 | CID 23,261,967 |
| CID 23,261,968 | CID 23,262,012 | CID 23,262,763 | CID 23,263,174 | CID 23,263,178 | CID 23,263,198 |
| CID 23,263,199 | CID 23,263,239 | CID 23,270,086 | CID 23,280,718 | CID 23,354,875 | CID 23,412,685 |
| CID 23,412,687 | CID 23,412,688 | CID 23,412,690 | CID 23,412,691 | CID 23,412,692 | CID 23,412,693 |
| CID 23,412,694 | CID 23,412,695 | CID 23,412,696 | CID 23,412,697 | CID 23,412,725 | CID 23,412,739 |
| CID 23,412,743 | CID 23,416,402 | CID 23,418,415 | CID 23,419,990 | CID 23,420,083 | CID 23,424,003 |
| CID 23,424,004 | CID 23,443,680 | CID 23,588,764 | CID 23,588,768 | CID 23,616,631 | CID 23,616,677 |
| CID 23,618,364 | CID 23,618,423 | CID 23,618,916 | CID 23,618,935 | CID 23,618,936 | CID 23,618,946 |
| CID 23,620,087 | CID 23,620,337 | CID 23,620,895 | CID 23,620,896 | CID 23,620,897 | CID 23,623,772 |
| CID 23,665,008 | CID 23,668,346 | CID 23,696,518 | CID 23,697,318 | CID 23,719,535 | CID 23,719,541 |
| CID 23,719,552 | CID 24,738,960 | CID 24,846,178 | CID 24,884,190 | CID 25,113,239 | CID 25,147,458 |
| CID 25,147,462 | CID 25,147,473 | CID 25,208,316 | CID 42,615,748 | CID 42,627,489 | CID 42,627,490 |
| CID 42,627,491 | CID 42,627,492 | CID 44,144,454 | CID 44,151,659 | CID 44,152,997 | CID 44,153,294 |
| CID 44,395,086 | CID 44,563,905 | CID 45,051,723 | CID 45,479,364 | CID 45,479,365 | CID 45,479,524 |
| CID 45,479,525 | CID 49,866,861 | CID 50,922,181 | CID 50,922,452 | CID 50,931,488 | CID 50,931,515 |
| CID 50,931,522 | CID 50,933,634 | CID 50,933,939 | CID 50,934,000 | CID 50,936,983 | CID 53,239,239 |
| CID 53,249,217 | CID 53,339,027 | CID 53,339,028 | CID 53,349,346 | CID 53,349,347 | CID 53,349,348 |
| CID 53,349,349 | CID 53,349,350 | CID 53,349,351 | CID 53,349,352 | CID 53,423,860 | CID 53,432,730 |
| CID 53,471,870 | CID 53,492,526 | CID 54,603,741 | CID 54,611,691 | CID 54,696,380 | CID 54,723,496 |
| CID 56,840,839 | CID 57,346,053 | CID 57,346,345 | CID 57,346,984 | CID 57,347,790 | CID 57,351,126 |
| CID 57,351,127 | CID 57,354,213 | CID 57,354,214 | CID 57,359,120 | CID 57,359,121 | CID 57,376,611 |
| CID 57,376,762 | CID 57,448,761 | CID 57,448,769 | CID 57,448,818 | CID 57,448,860 | CID 57,466,141 |
| CID 57,467,921 | CID 57,484,655 | CID 57,517,166 | CID 57,763,376 | CID 57,763,404 | CID 58,743,125 |
| CID 58,857,119 | CID 59,080,074 | CID 60,207,945 | CID 71,301,049 | CID 71,309,374 | CID 71,310,805 |
| CID 71,310,806 | CID 71,334,864 | CID 71,336,057 | CID 71,339,387 | CID 71,345,105 | CID 71,349,031 |
| CID 71,350,771 | CID 71,350,783 | CID 71,353,378 | CID 71,353,379 | CID 71,353,399 | CID 71,355,900 |
| CID 71,355,927 | CID 71,357,772 | CID 71,358,656 | CID 71,358,665 | CID 71,358,778 | CID 71,359,001 |
| CID 71,359,074 | CID 71,361,408 | CID 71,361,450 | CID 71,363,675 | CID 71,367,036 | CID 71,367,054 |
| CID 71,367,078 | CID 71,367,094 | CID 71,367,112 | CID 71,367,851 | CID 71,370,608 | CID 71,377,166 |
| CID 71,377,167 | CID 71,380,410 | CID 71,386,575 | CID 71,389,205 | CID 71,389,206 | CID 71,389,207 |
| CID 71,389,208 | CID 71,395,187 | CID 71,395,375 | CID 71,396,322 | CID 71,401,400 | CID 71,401,410 |
| CID 71,407,586 | CID 71,410,644 | CID 71,410,645 | CID 71,417,968 | CID 71,417,969 | CID 71,430,842 |
| CID 71,436,452 | CID 71,443,407 | CID 71,443,408 | CID 71,446,592 | CID 71,446,618 | CID 71,446,960 |
| CID 71,526,741 | CID 71,580,995 | CID 71,586,773 | CID 72,720,419 | CID 73,351,195 | CID 73,357,742 |
| CID 73,415,825 | CID 73,894,232 | CID 73,894,234 | CID 74,765,958 | CID 74,935,276 | CID 76,871,762 |
| CID 76,963,989 | CID 76,966,440 | CID 78,060,866 | CID 78,061,006 | CID 78,062,686 | CID 78,063,158 |
| CID 78,063,159 | CID 78,066,382 | CID 78,066,392 | CID 78,070,721 | CID 85,571,114 | CID 85,577,990 |
| CID 85,605,969 | CID 85,607,971 | CID 85,609,099 | CID 85,612,664 | CID 85,613,640 | CID 85,632,096 |
| CID 85,756,556 | CID 85,775,826 | CID 85,779,417 | CID 85,792,258 | CID 85,793,306 | CID 85,808,862 |
| CID 85,824,116 | CID 85,850,779 | CID 85,850,780 | CID 85,850,781 | CID 85,850,782 | CID 85,850,783 |
| CID 85,850,785 | CID 85,850,786 | CID 85,850,789 | CID 85,850,790 | CID 85,850,792 | CID 85,884,398 |
| CID 85,911,906 | CID 85,977,561 | CID 85,994,471 | CID 86,013,960 | CID 86,014,167 | CID 86,065,878 |
| CID 86,126,557 | CID 86,154,041 | CID 86,159,344 | CID 86,172,918 | CID 86,176,315 | CID 86,205,935 |
| CID 86,249,175 | CID 86,717,687 | CID 87,090,222 | CID 87,094,975 | CID 87,109,955 | CID 87,111,979 |
| CID 87,118,008 | CID 87,144,977 | CID 87,144,978 | CID 87,189,618 | CID 87,205,247 | CID 87,205,248 |
| CID 87,234,713 | CID 87,246,827 | CID 87,248,447 | CID 87,255,851 | CID 87,255,859 | CID 87,255,872 |
| CID 87,255,873 | CID 87,255,874 | CID 87,255,878 | CID 87,255,879 | CID 87,255,885 | CID 87,255,886 |
| CID 87,410,957 | CID 87,472,810 | CID 87,472,812 | CID 87,473,759 | CID 87,474,264 | CID 87,474,374 |
| CID 87,474,643 | CID 87,474,789 | CID 87,475,307 | CID 87,475,822 | CID 87,475,824 | CID 87,475,926 |
| CID 87,476,165 | CID 87,476,184 | CID 87,476,290 | CID 87,476,403 | CID 87,476,530 | CID 87,476,558 |
| CID 87,476,621 | CID 87,476,845 | CID 87,476,848 | CID 87,477,042 | CID 87,477,044 | CID 87,477,165 |
| CID 87,477,318 | CID 87,477,537 | CID 87,477,756 | CID 87,477,757 | CID 87,478,031 | CID 87,484,462 |
| CID 87,551,850 | CID 87,562,825 | CID 87,581,052 | CID 87,581,053 | CID 87,676,186 | CID 87,700,226 |
| CID 87,700,230 | CID 87,700,407 | CID 87,776,602 | CID 87,812,518 | CID 87,858,508 | CID 87,980,104 |
| CID 87,980,186 | CID 87,989,828 | CID 88,144,816 | CID 88,162,162 | CID 88,184,704 | CID 88,248,144 |
| CID 88,263,967 | CID 88,266,006 | CID 88,305,231 | CID 88,305,232 | CID 88,315,700 | CID 88,342,746 |
| CID 88,389,746 | CID 88,415,561 | CID 88,438,797 | CID 88,438,798 | CID 88,464,855 | CID 88,464,856 |
| CID 88,491,208 | CID 88,510,137 | CID 88,510,138 | CID 88,510,139 | CID 88,510,140 | CID 88,513,649 |
| CID 88,513,650 | CID 88,513,651 | CID 88,625,918 | CID 88,625,919 | CID 88,633,062 | CID 88,658,127 |
| CID 88,686,405 | CID 88,720,065 | CID 88,738,074 | CID 88,738,075 | CID 88,738,076 | CID 88,738,077 |
| CID 88,738,154 | CID 88,744,197 | CID 88,791,225 | CID 88,798,792 | CID 88,798,793 | CID 88,807,785 |
| CID 88,814,284 | CID 88,838,825 | CID 88,838,826 | CID 89,525,461 | CID 89,588,457 | CID 89,921,988 |
| CID 90,115,830 | CID 90,470,710 | CID 90,479,326 | CID 91,733,954 | CID 91,750,137 | CID 91,750,137 |
| CID 91,751,251 | CID 91,886,304 | CID 91,988,436 | CID 91,989,816 | CID 91,994,839 | CID 31,999,513 |
| CID 91,999,513 | CID 92,001,091 | CID 92,001,092 | CID 92,002,930 | CID 92,003,357 | CID 32,007,238 |
| CID 92,024,388 | CID 92,024,388 | CID 92,024,399 | CID 92,024,401 | CID 92,024,402 | CID 92,025,101 |
| CID 92,025,102 | CID 92,025,103 | CID 92,025,104 | CID 92,025,105 | CID 92,025,106 | CID 92,025,107 |
| CID 92,025,108 | CID 92,025,109 | CID 92,025,110 | CID 92,025,111 | CID 92,025,112 | CID 92,025,113 |
| CID 92,025,732 | CID 92,026,266 | CID 92,026,424 | CID 92,026,427 | CID 92,026,688 | CID 92,026,716 |
| CID 92,026,737 | CID 92,026,781 | CID 92,026,881 | CID 92,026,963 | CID 92,027,058 | CID 92,027,110 |
| CID 92,027,269 | CID 92,027,367 | CID 92,027,458 | CID 92,027,511 | CID 92,027,541 | CID 92,027,722 |
| CID 92,028,182 | CID 92,028,237 | CID 92,028,421 | CID 92,028,660 | CID 92,028,686 | CID 32,028,728 |
| CID 92,028,791 | CID 92,028,795 | CID 92,028,877 | CID 92,028,881 | CID 92,028,938 | CID 92,043,202 |
| CID 100,918,999 | CID 100,919,000 | CID 100,942,061 | CID 100,942,062 | CID 100,949,123 | CID 100,986,766 |
| CID 100,986,767 | CID 100,991,044 | CID 100,991,045 | CID 100,999,027 | CID 101,005,335 | CID 101,005,336 |
| CID 101,005,337 | CID 101,005,338 | CID 101,005,339 | CID 101,013,781 | CID 101,013,782 | CID 101,013,783 |
| CID 101,047,156 | CID 101,064,325 | CID 101,085,742 | CID 101,113,845 | CID 101,113,846 | CID 101,116,387 |
| CID 101,117,156 | CID 101,118,991 | CID 101,174,827 | CID 101,237,011 | CID 101,260,419 | CID 101,282,796 |
| CID 101,289,874 | CID 101,354,116 | CID 101,354,274 | CID 101,379,755 | CID 101,411,205 | CID 101,436,717 |
| CID 101,436,720 | CID 101,458,326 | CID 101,459,329 | CID 101,485,971 | CID 101,485,973 | CID 101,485,974 |
| CID 101,499,194 | CID 101,499,195 | CID 101,533,802 | CID 101,533,803 | CID 101,547,226 | CID 101,575,977 |
| CID 101,617,225 | CID 101,619,265 | CID 101,666,991 | CID 101,666,992 | CID 101,682,434 | CID 101,682,435 |
| CID 101,682,436 | CID 101,682,437 | CID 101,682,438 | CID 101,694,974 | CID 101,694,975 | CID 101,694,976 |
| CID 101,694,977 | CID 101,694,978 | CID 101,694,979 | CID 101,695,476 | CID 101,763,094 | CID 101,763,095 |
| CID 101,763,095 | CID 101,763,096 | CID 101,763,096 | CID 101,763,097 | CID 101,763,098 | CID 101,817,035 |
| CID 101,838,903 | CID 101,838,904 | CID 101,848,513 | CID 101,852,195 | CID 101,861,498 | CID 101,861,499 |
| CID 101,913,584 | CID 101,923,897 | CID 101,948,144 | CID 102,015,291 | CID 102,034,400 | CID 102,062,180 |
| CID 102,062,402 | CID 102,063,353 | CID 102,076,551 | CID 102,076,552 | CID 102,095,097 | CID 102,098,989 |
| CID 102,098,990 | CID 102,167,962 | CID 102,180,144 | CID 102,180,145 | CID 102,186,306 | CID 102,213,935 |
| CID 102,213,936 | CID 102,213,937 | CID 102,226,697 | CID 102,239,094 | CID 102,351,891 | CID 102,351,892 |
| CID 102,351,897 | CID 102,351,898 | CID 102,371,199 | CID 102,414,495 | CID 102,414,496 | CID 102,415,253 |
| CID 102,496,603 | CID 102,499,917 | CID 102,499,918 | CID 102,511,469 | CID 102,528,387 | CID 102,528,388 |
| CID 102,528,389 | CID 102,528,390 | CID 102,593,935 | CID 102,601,572 | CID 119,077,607 | CID 122,364,373 |
| CID 122,378,045 | CID 122,378,046 | CID 123,133,548 | CID 123,133,548 | CID 129,628,305 | CID 125,629,427 |
| CID 129,630,515 | CID 129,631,280 | CID 129,631,344 | CID 129,631,597 | CID 129,631,743 | CID 129,631,758 |
| CID 129,631,787 | CID 129,631,891 | CID 129,634,318 | CID 129,635,174 | CID 129,637,470 | CID 129,642,045 |
| CID 129,642,046 | CID 129,643,934 | CID 129,643,936 | CID 129,643,938 | CID 129,645,426 | CID 129,651,528 |
| CID 129,652,225 | CID 129,655,074 | CID 129,659,469 | CID 123,660,712 | CID 129,672,983 | CID 129,677,715 |
| CID 129,679,346 | CID 129,679,347 | CID 129,679,425 | CID 129,679,623 | CID 129,680,077 | CID 129,686,362 |
| CID 129,689,790 | CID 129,692,591 | CID 129,693,147 | CID 129,703,372 | CID 129,703,373 | CID 129,705,075 |
| CID 129,705,678 | CID 129,715,852 | CID 129,724,489 | CID 129,729,547 | CID 129,729,551 | CID 129,737,952 |
| CID 129,741,926 | CID 129,742,025 | CID 129,742,535 | CID 129,756,855 | CID 129,757,023 | CID 129,757,215 |
| CID 129,774,469 | CID 129,774,997 | CID 129,805,810 | CID 129,809,454 | CID 129,821,735 | CID 129,828,133 |
| CID 129,828,662 | CID 129,828,842 | CID 129,828,911 | CID 129,829,589 | CID 129,842,137 | CID 129,842,236 |
| CID 129,842,237 | CID 129,849,818 | CID 129,858,925 | CID 129,858,931 | CID 129,863,921 | CID 129,865,429 |
| CID 129,865,431 | CID 129,873,049 | CID 129,887,306 | CID 129,888,984 | CID 129,889,538 | CID 129,890,164 |
| CID 129,891,119 | CID 129,891,127 | CID 129,891,128 | CID 129,892,162 | CID 131,700,384 | CID 131,736,786 |
| CID 131,852,787 | CID 131,852,788 | CID 131,864,457 | CID 131,864,465 | CID 131,864,960 | CID 131,865,018 |
| CID 131,871,206 | CID 131,872,531 | CID 131,874,138 | CID 131,874,746 | CID 131,876,990 | CID 131,876,991 |
| CID 131,876,992 | CID 131,884,149 | | | | |

TABLE 2
3071 five valence arsenic (As) containing compounds were predicted to efficiently bind PANDA Pocket and efficiently rescue structural mp53. All of the 94.2 million structures recorded in PubChem (https://pubchem.ncbi.nlm.nih.gov/) were applied for 4C+ screening. In the 4C+ screening, we collected those with more than 2 cysteine-binding potential. Carbon-binding As/Sb/Bi bond has defect in binding cysteine since this bond cannot be hydrolyzed. The other As/Sb/Bi bond can be hydrolyzed in cells and thus is able to bind cysteine.
Arsenic 5 Valence PANDA Agents

| | | | | | |
|---|---|---|---|---|---|
| CID 233 | CID 234 | CID 1,985 | CID 2,513 | CID 5,104 | CID 7,365 |
| CID 7,373 | CID 7,389 | CID 8,312 | CID 8,480 | CID 8,802 | CID 8,947 |
| CID 8,948 | CID 9,313 | CID 9,314 | CID 9,525 | CID 10,799 | CID 10,809 |
| CID 10,810 | CID 11,453 | CID 12,055 | CID 13,127 | CID 13,479 | CID 14,771 |
| CID 15,465 | CID 16,555 | CID 16,851 | CID 17,845 | CID 20,770 | CID 21,074 |
| CID 21,250 | CID 21,251 | CID 21,252 | CID 21,253 | CID 21,511 | CID 21,548 |
| CID 21,549 | CID 22,193 | CID 22,273 | CID 22,773 | CID 22,874 | CID 23,060 |
| CID 23,129 | CID 23,514 | CID 23,515 | CID 23,749 | CID 24,262 | CID 24,500 |
| CID 24,501 | CID 24,572 | CID 24,574 | CID 24,938 | CID 24,943 | CID 25,303 |
| CID 25,929 | CID 26,065 | CID 27,052 | CID 27,401 | CID 27,817 | CID 30,852 |
| CID 30,853 | CID 30,854 | CID 32,602 | CID 36,829 | CID 38,223 | CID 44,902 |
| CID 45,720 | CID 46,313 | CID 46,681 | CID 46,683 | CID 46,684 | CID 46,685 |
| CID 46,686 | CID 46,840 | CID 47,275 | CID 52,340 | CID 52,503 | CID 52,504 |
| CID 52,505 | CID 52,506 | CID 52,949 | CID 52,950 | CID 52,951 | CID 52,952 |
| CID 52,953 | CID 52,954 | CID 61,460 | CID 61,477 | CID 61,617 | CID 61,618 |
| CID 61,619 | CID 62,043 | CID 66,826 | CID 66,944 | CID 66,945 | CID 67,177 |
| CID 67,178 | CID 67,782 | CID 68,198 | CID 68,199 | CID 68,532 | CID 68,716 |
| CID 72,905 | CID 73,161 | CID 74,471 | CID 77,393 | CID 77,588 | CID 77,589 |
| CID 79,424 | CID 79,425 | CID 79,502 | CID 79,769 | CID 79,849 | CID 79,871 |
| CID 80,448 | CID 80,568 | CID 80,894 | CID 81,422 | CID 82,223 | CID 82,228 |
| CID 84,776 | CID 93,839 | CID 94,822 | CID 94,911 | CID 94,933 | CID 95,017 |
| CID 95,018 | CID 95,019 | CID 95,452 | CID 95,470 | CID 104,785 | CID 108,900 |
| CID 113,151 | CID 113,152 | CID 114,800 | CID 115,320 | CID 115,371 | CID 116,251 |
| CID 116,491 | CID 116,504 | CID 119,656 | CID 120,177 | CID 124,738 | CID 124,739 |
| CID 124,921 | CID 126,669 | CID 127,752 | CID 128,033 | CID 129,501 | CID 135,804 |
| CID 135,805 | CID 135,806 | CID 145,014 | CID 145,125 | CID 151,614 | CID 151,615 |
| CID 151,637 | CID 156,005 | CID 159,810 | CID 160,269 | CID 160,801 | CID 161,792 |
| CID 165,585 | CID 166,814 | CID 166,815 | CID 166,826 | CID 166,828 | CID 166,835 |
| CID 167,250 | CID 167,617 | CID 167,906 | CID 169,458 | CID 173,497 | CID 174,008 |
| CID 174,227 | CID 176,957 | CID 177,152 | CID 177,517 | CID 177,518 | CID 177,719 |
| CID 178,410 | CID 178,417 | CID 178,418 | CID 182,380 | CID 185,520 | CID 187,828 |
| CID 187,863 | CID 187,864 | CID 189,991 | CID 189,992 | CID 190,282 | CID 192,901 |
| CID 193,598 | CID 193,853 | CID 196,505 | CID 197,032 | CID 197,906 | CID 201,395 |
| CID 202,213 | CID 202,581 | CID 202,583 | CID 203,113 | CID 203,535 | CID 203,784 |
| CID 219,617 | CID 220,597 | CID 220,599 | CID 220,807 | CID 222,574 | CID 223,334 |
| CID 223,335 | CID 223,336 | CID 223,337 | CID 223,338 | CID 223,339 | CID 223,342 |
| CID 223,343 | CID 223,344 | CID 223,345 | CID 223,347 | CID 223,349 | CID 223,352 |
| CID 223,788 | CID 223,789 | CID 223,806 | CID 223,807 | CID 224,248 | CID 224,249 |
| CID 224,250 | CID 224,251 | CID 224,252 | CID 224,253 | CID 224,255 | CID 224,256 |
| CID 224,257 | CID 224,258 | CID 224,260 | CID 224,261 | CID 224,262 | CID 224,263 |
| CID 224,264 | CID 224,265 | CID 224,267 | CID 224,268 | CID 224,270 | CID 224,271 |
| CID 224,272 | CID 224,273 | CID 224,274 | CID 224,275 | CID 224,276 | CID 224,278 |
| CID 224,282 | CID 224,283 | CID 224,284 | CID 224,288 | CID 224,289 | CID 224,290 |
| CID 224,294 | CID 224,295 | CID 224,298 | CID 224,301 | CID 224,309 | CID 224,575 |
| CID 224,660 | CID 224,877 | CID 224,878 | CID 224,880 | CID 224,881 | CID 224,891 |
| CID 224,892 | CID 224,893 | CID 224,894 | CID 224,896 | CID 224,897 | CID 224,901 |
| CID 224,902 | CID 224,903 | CID 224,906 | CID 224,907 | CID 224,908 | CID 224,911 |
| CID 224,912 | CID 224,914 | CID 224,916 | CID 224,917 | CID 224,918 | CID 224,920 |
| CID 224,921 | CID 224,922 | CID 224,923 | CID 224,924 | CID 224,925 | CID 224,927 |
| CID 224,928 | CID 224,929 | CID 225,780 | CID 225,783 | CID 225,785 | CID 225,786 |
| CID 225,788 | CID 225,789 | CID 225,791 | CID 225,798 | CID 225,799 | CID 225,802 |
| CID 225,806 | CID 229,965 | CID 231,057 | CID 231,882 | CID 231,895 | CID 231,896 |
| CID 231,966 | CID 231,967 | CID 231,968 | CID 231,969 | CID 232,560 | CID 232,563 |
| CID 232,565 | CID 232,566 | CID 232,567 | CID 233,201 | CID 233,281 | CID 233,320 |
| CID 233,321 | CID 233,324 | CID 233,325 | CID 233,833 | CID 234,155 | CID 234,202 |
| CID 234,284 | CID 234,285 | CID 234,286 | CID 234,535 | CID 234,536 | CID 234,539 |
| CID 234,557 | CID 234,559 | CID 234,560 | CID 234,637 | CID 234,639 | CID 234,890 |
| CID 236,817 | CID 237,668 | CID 237,669 | CID 237,670 | CID 237,671 | CID 237,672 |
| CID 237,673 | CID 237,674 | CID 237,991 | CID 238,189 | CID 238,195 | CID 238,219 |
| CID 238,220 | CID 238,221 | CID 238,223 | CID 238,243 | CID 238,244 | CID 239,021 |
| CID 239,026 | CID 239,027 | CID 239,028 | CID 239,032 | CID 240,762 | CID 241,152 |
| CID 241,153 | CID 241,156 | CID 241,157 | CID 241,158 | CID 241,631 | CID 241,636 |
| CID 241,906 | CID 241,907 | CID 241,908 | CID 241,909 | CID 241,911 | CID 241,912 |
| CID 241,913 | CID 241,918 | CID 241,958 | CID 241,963 | CID 241,964 | CID 244,263 |
| CID 244,264 | CID 244,266 | CID 247,798 | CID 247,799 | CID 247,800 | CID 251,060 |
| CID 252,526 | CID 256,154 | CID 258,099 | CID 260,823 | CID 260,832 | CID 261,003 |
| CID 261,005 | CID 261,009 | CID 261,010 | CID 261,011 | CID 261,012 | CID 261,013 |
| CID 261,014 | CID 261,015 | CID 261,016 | CID 261,020 | CID 261,021 | CID 261,022 |
| CID 261,023 | CID 261,024 | CID 261,025 | CID 261,027 | CID 261,032 | CID 261,033 |
| CID 261,034 | CID 261,035 | CID 261,041 | CID 261,042 | CID 261,044 | CID 261,045 |
| CID 261,049 | CID 261,050 | CID 261,227 | CID 261,292 | CID 263,033 | CID 266,086 |
| CID 266,087 | CID 266,088 | CID 266,089 | CID 266,090 | CID 266,091 | CID 266,092 |
| CID 266,093 | CID 266,094 | CID 266,095 | CID 266,096 | CID 266,097 | CID 266,098 |
| CID 266,099 | CID 266,100 | CID 266,101 | CID 267,079 | CID 267,080 | CID 267,081 |
| CID 267,082 | CID 267,083 | CID 267,084 | CID 267,085 | CID 267,086 | CID 267,087 |
| CID 267,088 | CID 268,483 | CID 269,333 | CID 269,334 | CID 269,335 | CID 269,336 |
| CID 269,337 | CID 271,056 | CID 272,658 | CID 272,661 | CID 276,766 | CID 279,132 |
| CID 279,133 | CID 279,134 | CID 279,135 | CID 279,136 | CID 279,139 | CID 279,140 |
| CID 279,143 | CID 279,145 | CID 284,458 | CID 285,210 | CID 290,150 | CID 291,940 |
| CID 302,275 | CID 302,501 | CID 306,424 | CID 311,059 | CID 312,296 | CID 312,297 |
| CID 312,298 | CID 312,299 | CID 312,300 | CID 312,301 | CID 312,302 | CID 312,303 |
| CID 312,304 | CID 312,305 | CID 312,306 | CID 312,307 | CID 312,308 | CID 312,309 |
| CID 312,310 | CID 312,311 | CID 312,312 | CID 312,313 | CID 312,314 | CID 312,315 |
| CID 312,316 | CID 312,317 | CID 312,318 | CID 312,319 | CID 312,320 | CID 312,321 |
| CID 312,323 | CID 312,324 | CID 312,325 | CID 312,326 | CID 312,327 | CID 312,328 |
| CID 312,329 | CID 312,343 | CID 312,344 | CID 312,345 | CID 312,346 | CID 312,347 |
| CID 312,348 | CID 312,349 | CID 312,350 | CID 312,351 | CID 312,352 | CID 312,353 |
| CID 312,354 | CID 312,355 | CID 312,356 | CID 312,357 | CID 312,358 | CID 312,359 |
| CID 312,360 | CID 312,361 | CID 312,362 | CID 312,363 | CID 312,364 | CID 312,365 |
| CID 312,366 | CID 312,367 | CID 312,368 | CID 312,369 | CID 312,370 | CID 312,371 |
| CID 312,372 | CID 312,373 | CID 312,374 | CID 312,398 | CID 312,399 | CID 313,155 |
| CID 313,862 | CID 313,863 | CID 313,302 | CID 313,903 | CID 313,904 | CID 314,151 |
| CID 315,973 | CID 315,974 | CID 316,130 | CID 316,131 | CID 316,132 | CID 316,133 |
| CID 320,045 | CID 320,046 | CID 336,175 | CID 342,404 | CID 343,608 | CID 343,707 |
| CID 343,967 | CID 345,305 | CID 345,306 | CID 345,307 | CID 345,308 | CID 348,593 |
| CID 348,594 | CID 349,408 | CID 349,409 | CID 349,410 | CID 349,411 | CID 349,412 |
| CID 351,639 | CID 352,197 | CID 352,198 | CID 352,199 | CID 352,200 | CID 352,201 |
| CID 370,185 | CID 370,186 | CID 370,187 | CID 370,188 | CID 375,754 | CID 375,755 |
| CID 375,756 | CID 375,757 | CID 375,758 | CID 375,759 | CID 408,050 | CID 408,051 |
| CID 408,054 | CID 408,195 | CID 408,316 | CID 408,516 | CID 408,517 | CID 408,519 |
| CID 408,520 | CID 408,521 | CID 408,738 | CID 408,750 | CID 408,799 | CID 412,896 |
| CID 416,469 | CID 416,470 | CID 416,681 | CID 416,682 | CID 417,079 | CID 418,983 |
| CID 419,695 | CID 421,932 | CID 427,855 | CID 427,856 | CID 427,857 | CID 428,063 |
| CID 437,230 | CID 437,231 | CID 444,541 | CID 448,676 | CID 459,200 | CID 459,202 |
| CID 459,204 | CID 459,205 | CID 459,206 | CID 459,207 | CID 459,208 | CID 459,210 |
| CID 459,212 | CID 459,213 | CID 459,215 | CID 459,218 | CID 459,219 | CID 459,220 |
| CID 459,221 | CID 459,222 | CID 459,223 | CID 459,224 | CID 459,225 | CID 504,158 |
| CID 516,879 | CID 516,880 | CID 516,881 | CID 516,883 | CID 517,279 | CID 517,280 |
| CID 518,706 | CID 520,618 | CID 535,512 | CID 579,327 | CID 579,570 | CID 586,279 |
| CID 588,512 | CID 607,978 | CID 608,863 | CID 614,819 | CID 617,794 | CID 618,159 |
| CID 618,262 | CID 618,397 | CID 620,441 | CID 624,094 | CID 2,724,247 | CID 2,724,695 |
| CID 2,737,135 | CID 2,750,690 | CID 3,026,915 | CID 3,027,197 | CID 3,027,198 | CID 3,027,199 |
| CID 3,027,200 | CID 3,027,201 | CID 3,027,202 | CID 3,027,203 | CID 3,027,204 | CID 3,027,205 |
| CID 3,027,206 | CID 3,027,207 | CID 3,027,208 | CID 3,027,209 | CID 3,027,210 | CID 3,027,211 |
| CID 3,027,212 | CID 3,027,213 | CID 3,027,214 | CID 3,027,215 | CID 3,027,216 | CID 3,032,448 |
| CID 3,045,788 | CID 3,048,851 | CID 3,049,066 | CID 3,056,312 | CID 3,056,538 | CID 3,056,539 |
| CID 3,066,278 | CID 3,080,685 | CID 3,084,152 | CID 3,084,166 | CID 3,246,035 | CID 3,246,047 |
| CID 3,246,304 | CID 3,246,400 | CID 3,280,085 | CID 3,286,756 | CID 3,286,757 | CID 3,302,537 |
| CID 3,371,533 | CID 3,475,487 | CID 3,492,319 | CID 3,509,539 | CID 3,511,008 | CID 3,531,782 |
| CID 3,562,037 | CID 3,597,904 | CID 3,665,457 | CID 3,752,912 | CID 3,809,018 | CID 4,027,696 |
| CID 4,116,415 | CID 4,144,350 | CID 4,147,508 | CID 4,164,893 | CID 4,179,826 | CID 4,191,628 |
| CID 4,199,732 | CID 4,206,495 | CID 4,246,481 | CID 4,248,649 | CID 4,340,488 | CID 4,383,733 |
| CID 4,389,454 | CID 4,412,562 | CID 4,435,830 | CID 4,459,508 | CID 4,486,063 | CID 4,549,863 |
| CID 4,584,095 | CID 4,607,773 | CID 4,619,461 | CID 4,644,413 | CID 4,650,712 | CID 4,685,638 |
| CID 4,993,357 | CID 5,009,787 | CID 5,080,432 | CID 5,107,483 | CID 5,142,636 | CID 5,162,080 |
| CID 5,167,687 | CID 5,232,584 | CID 5,232,585 | CID 5,238,192 | CID 5,239,771 | CID 5,247,890 |
| CID 5,250,553 | CID 5,251,787 | CID 5,252,153 | CID 5,256,281 | CID 5,256,284 | CID 5,259,384 |
| CID 5,351,644 | CID 5,351,887 | CID 5,351,895 | CID 5,351,952 | CID 5,354,537 | CID 5,354,538 |
| CID 5,354,540 | CID 5,354,630 | CID 5,355,552 | CID 5,355,760 | CID 5,357,528 | CID 5,357,558 |
| CID 5,357,633 | CID 5,357,680 | CID 5,357,681 | CID 5,358,126 | CID 5,359,629 | CID 5,360,419 |
| CID 5,380,357 | CID 5,381,267 | CID 5,382,968 | CID 5,459,312 | CID 5,459,316 | CID 5,460,560 |
| CID 5,460,568 | CID 5,460,585 | CID 5,460,701 | CID 5,464,162 | CID 5,464,667 | CID 5,473,608 |
| CID 5,475,118 | CID 5,475,577 | CID 5,476,381 | CID 5,476,572 | CID 5,483,153 | CID 5,483,161 |
| CID 5,489,080 | CID 5,489,145 | CID 5,491,976 | CID 5,492,518 | CID 5,702,671 | CID 5,748,340 |
| CID 5,748,677 | CID 5,821,031 | CID 5,826,948 | CID 5,829,414 | CID 5,880,406 | CID 5,937,425 |
| CID 5,959,443 | CID 5,992,379 | CID 6,049,608 | CID 6,070,355 | CID 6,080,553 | CID 6,087,455 |
| CID 6,096,941 | CID 6,109,063 | CID 6,159,957 | CID 6,312,763 | CID 6,327,082 | CID 6,327,913 |
| CID 6,328,535 | CID 6,328,560 | CID 6,330,351 | CID 6,331,888 | CID 6,332,329 | CID 6,337,418 |
| CID 6,364,518 | CID 6,364,519 | CID 6,364,520 | CID 6,364,651 | CID 6,366,769 | CID 6,376,379 |
| CID 6,382,968 | CID 6,394,013 | CID 6,395,068 | CID 6,395,320 | CID 6,395,368 | CID 6,396,200 |
| CID 6,396,201 | CID 6,418,871 | CID 6,432,805 | CID 6,433,170 | CID 6,433,171 | CID 6,433,174 |
| CID 6,437,949 | CID 6,444,039 | CID 6,449,838 | CID 6,451,222 | CID 6,451,314 | CID 6,451,315 |
| CID 6,452,870 | CID 6,453,098 | CID 6,454,696 | CID 6,455,149 | CID 6,455,217 | CID 6,507,977 |
| CID 6,508,406 | CID 6,508,670 | CID 6,521,590 | CID 6,523,657 | CID 6,537,679 | CID 6,713,513 |
| CID 6,713,772 | CID 6,798,087 | CID 6,799,026 | CID 6,802,412 | CID 6,811,780 | CID 6,814,348 |
| CID 6,817,904 | CID 6,825,298 | CID 6,833,367 | CID 6,833,821 | CID 6,911,855 | CID 9,543,168 |
| CID 9,548,831 | CID 9,548,866 | CID 9,548,867 | CID 9,575,228 | CID 9,575,832 | CID 9,577,326 |
| CID 9,577,343 | CID 9,578,161 | CID 9,578,311 | CID 9,578,314 | CID 9,580,365 | CID 9,589,372 |
| CID 9,589,414 | CID 9,604,595 | CID 9,605,082 | CID 9,649,548 | CID 9,798,053 | CID 9,808,042 |
| CID 9,810,878 | CID 9,824,749 | CID 9,837,036 | CID 9,860,187 | CID 9,860,551 | CID 9,862,801 |
| CID 9,865,965 | CID 9,888,902 | CID 9,931,103 | CID 9,936,030 | CID 9,949,579 | CID 9,949,580 |
| CID 9,954,856 | CID 10,023,468 | CID 10,024,149 | CID 10,069,126 | CID 10,090,976 | CID 10,257,888 |
| CID 10,312,004 | CID 10,338,262 | CID 10,345,828 | CID 10,349,701 | CID 10,386,772 | CID 10,394,431 |
| CID 10,431,022 | CID 10,443,824 | CID 10,477,524 | CID 10,505,812 | CID 10,508,513 | CID 10,530,076 |
| CID 10,577,925 | CID 10,643,908 | CID 10,649,763 | CID 10,650,069 | CID 10,651,033 | CID 10,666,444 |
| CID 10,671,140 | CID 10,674,237 | CID 10,697,032 | CID 10,744,586 | CID 10,745,819 | CID 10,746,567 |
| CID 10,748,489 | CID 10,757,607 | CID 10,816,826 | CID 10,834,120 | CID 10,840,702 | CID 10,876,483 |
| CID 10,878,406 | CID 10,878,407 | CID 10,936,360 | CID 10,981,618 | CID 11,002,054 | CID 11,005,114 |
| CID 11,049,085 | CID 11,063,982 | CID 11,077,325 | CID 11,103,145 | CID 11,187,997 | CID 11,195,304 |
| CID 11,211,437 | CID 11,224,375 | CID 11,232,487 | CID 11,251,075 | CID 11,269,492 | CID 11,311,393 |
| CID 11,334,638 | CID 11,336,975 | CID 11,369,488 | CID 11,371,427 | CID 11,403,366 | CID 11,435,113 |
| CID 11,452,073 | CID 11,468,263 | CID 11,559,479 | CID 11,560,431 | CID 11,624,317 | CID 11,738,886 |
| CID 11,750,101 | CID 11,800,790 | CID 11,801,096 | CID 11,813,954 | CID 11,813,954 | CID 11,949,825 |
| CID 11,949,826 | CID 11,949,827 | CID 11,949,992 | CID 11,949,993 | CID 11,949,994 | CID 11,949,995 |
| CID 11,949,996 | CID 11,949,997 | CID 11,950,160 | CID 11,952,475 | CID 11,952,476 | CID 11,952,477 |
| CID 11,971,649 | CID 11,971,650 | CID 11,980,558 | CID 11,985,990 | CID 11,986,029 | CID 11,987,483 |
| CID 12,049,081 | CID 12,049,082 | CID 12,049,083 | CID 12,112,343 | CID 12,305,258 | CID 12,305,260 |
| CID 12,485,864 | CID 12,485,867 | CID 12,485,868 | CID 12,485,869 | CID 12,485,870 | CID 12,595,784 |
| CID 12,645,905 | CID 12,654,670 | CID 12,736,159 | CID 12,736,220 | CID 12,747,453 | CID 12,756,830 |
| CID 12,795,887 | CID 12,824,390 | CID 12,832,980 | CID 12,832,981 | CID 12,884,523 | CID 13,047,346 |
| CID 13,050,612 | CID 13,134,261 | CID 13,212,303 | CID 13,212,306 | CID 13,212,312 | CID 13,302,718 |
| CID 13,302,719 | CID 13,416,700 | CID 13,443,227 | CID 13,443,228 | CID 13,516,476 | CID 13,613,089 |
| CID 13,643,880 | CID 13,678,819 | CID 13,678,824 | CID 13,678,825 | CID 13,683,693 | CID 13,726,122 |
| CID 13,734,063 | CID 13,743,985 | CID 13,750,255 | CID 13,765,615 | CID 13,765,616 | CID 13,805,684 |
| CID 13,805,693 | CID 13,812,675 | CID 13,813,546 | CID 13,829,286 | CID 13,909,037 | CID 13,955,960 |
| CID 13,980,772 | CID 13,981,841 | CID 14,009,685 | CID 14,066,966 | CID 14,066,967 | CID 14,094,413 |
| CID 14,122,911 | CID 14,178,438 | CID 14,205,366 | CID 14,205,726 | CID 14,205,728 | CID 14,205,730 |
| CID 14,205,731 | CID 14,223,279 | CID 14,389,153 | CID 14,401,159 | CID 14,401,162 | CID 14,453,762 |
| CID 14,513,880 | CID 14,671,602 | CID 14,700,088 | CID 14,744,953 | CID 14,794,649 | CID 14,818,755 |
| CID 14,833,399 | CID 14,876,330 | CID 14,876,967 | CID 14,902,819 | CID 14,912,293 | CID 14,921,144 |
| CID 15,001,088 | CID 15,012,428 | CID 15,072,186 | CID 15,211,288 | CID 15,225,244 | CID 15,225,439 |
| CID 15,360,813 | CID 15,362,043 | CID 15,394,753 | CID 15,412,253 | CID 15,412,285 | CID 15,412,290 |
| CID 15,535,760 | CID 15,536,995 | CID 15,688,459 | CID 15,787,841 | CID 15,796,321 | CID 16,211,184 |
| CID 16,211,720 | CID 16,213,153 | CID 16,682,839 | CID 16,683,367 | CID 16,684,317 | CID 16,685,520 |
| CID 16,685,995 | CID 16,696,582 | CID 16,697,858 | CID 16,697,859 | CID 16,697,860 | CID 16,697,861 |
| CID 16,697,862 | CID 16,697,863 | CID 16,703,059 | CID 16,704,216 | CID 16,704,429 | CID 16,704,431 |
| CID 16,705,057 | CID 16,714,200 | CID 16,714,201 | CID 17,753,879 | CID 17,753,924 | CID 17,972,451 |
| CID 18,006,662 | CID 18,362,488 | CID 18,401,442 | CID 18,426,515 | CID 18,442,047 | CID 18,458,521 |
| CID 18,471,244 | CID 18,513,358 | CID 18,513,372 | CID 18,544,746 | CID 18,620,404 | CID 18,620,407 |
| CID 18,633,056 | CID 18,633,058 | CID 18,678,997 | CID 18,690,655 | CID 18,699,569 | CID 18,760,606 |
| CID 18,760,618 | CID 18,760,669 | CID 18,762,179 | CID 18,764,458 | CID 18,785,193 | CID 18,785,194 |
| CID 18,798,734 | CID 18,942,540 | CID 18,942,541 | CID 18,944,705 | CID 18,950,086 | CID 18,956,772 |
| CID 18,981,320 | CID 18,982,476 | CID 18,982,574 | CID 19,023,078 | CID 19,026,428 | CID 19,026,430 |
| CID 19,026,432 | CID 19,026,436 | CID 19,026,437 | CID 19,026,438 | CID 19,026,441 | CID 19,032,110 |
| CID 19,044,210 | CID 19,067,415 | CID 19,077,038 | CID 19,105,252 | CID 19,347,785 | CID 19,350,187 |
| CID 19,360,272 | CID 19,382,081 | CID 19,609,626 | CID 19,609,639 | CID 19,702,616 | CID 19,739,449 |
| CID 19,739,453 | CID 19,739,455 | CID 19,748,686 | CID 19,751,144 | CID 19,757,136 | CID 19,757,143 |
| CID 19,757,145 | CID 19,757,146 | CID 19,762,759 | CID 19,786,074 | CID 19,790,659 | CID 19,795,274 |
| CID 19,827,519 | CID 19,833,769 | CID 19,836,514 | CID 19,872,105 | CID 19,882,894 | CID 19,893,232 |
| CID 19,898,354 | CID 19,968,620 | CID 19,981,220 | CID 20,031,692 | CID 20,035,298 | CID 20,035,299 |
| CID 20,038,204 | CID 20,042,688 | CID 20,054,866 | CID 20,054,981 | CID 20,055,037 | CID 20,055,252 |
| CID 20,056,599 | CID 20,056,600 | CID 20,056,786 | CID 20,063,715 | CID 20,063,742 | CID 20,068,039 |
| CID 20,083,766 | CID 20,083,785 | CID 20,083,796 | CID 20,083,797 | CID 20,085,186 | CID 20,095,232 |
| CID 20,143,803 | CID 20,144,054 | CID 20,152,548 | CID 20,152,549 | CID 20,152,701 | CID 20,154,552 |
| CID 20,202,607 | CID 20,202,656 | CID 20,207,034 | CID 20,209,276 | CID 20,209,388 | CID 20,211,642 |
| CID 20,246,464 | CID 20,271,624 | CID 20,287,272 | CID 20,288,961 | CID 20,360,926 | CID 20,371,762 |
| CID 20,445,407 | CID 20,474,394 | CID 20,474,403 | CID 20,474,405 | CID 20,480,188 | CID 20,482,994 |
| CID 20,484,753 | CID 20,486,182 | CID 20,488,746 | CID 20,503,507 | CID 20,503,509 | CID 20,503,511 |
| CID 20,503,512 | CID 20,503,514 | CID 20,512,586 | CID 20,516,635 | CID 20,529,308 | CID 20,531,312 |
| CID 20,542,969 | CID 20,549,509 | CID 20,558,446 | CID 20,569,120 | CID 20,634,001 | CID 20,693,889 |
| CID 20,832,953 | CID 20,836,092 | CID 20,837,621 | CID 20,838,029 | CID 20,838,030 | CID 20,838,042 |
| CID 20,841,764 | CID 20,841,765 | CID 20,844,702 | CID 20,844,703 | CID 20,845,094 | CID 20,845,106 |
| CID 20,849,356 | CID 20,975,696 | CID 20,977,035 | CID 20,977,049 | CID 20,978,299 | CID 21,114,753 |
| CID 21,114,754 | CID 21,114,763 | CID 21,114,764 | CID 21,114,783 | CID 21,114,860 | CID 21,115,629 |
| CID 21,117,047 | CID 21,119,891 | CID 21,120,402 | CID 21,120,735 | CID 21,120,889 | CID 21,120,928 |
| CID 21,121,096 | CID 21,121,135 | CID 21,122,097 | CID 21,125,668 | CID 21,126,516 | CID 21,127,315 |
| CID 21,127,316 | CID 21,127,707 | CID 21,139,502 | CID 21,139,503 | CID 21,139,534 | CID 21,139,535 |
| CID 21,141,080 | CID 21,141,081 | CID 21,152,149 | CID 21,154,358 | CID 21,183,466 | CID 21,225,639 |
| CID 21,225,707 | CID 21,252,377 | CID 21,252,378 | CID 21,321,841 | CID 21,391,529 | CID 21,453,257 |
| CID 21,455,200 | CID 21,459,652 | CID 21,491,032 | CID 21,491,033 | CID 21,528,715 | CID 21,536,734 |
| CID 21,536,760 | CID 21,536,766 | CID 21,536,770 | CID 21,536,773 | CID 21,536,775 | CID 21,536,777 |
| CID 21,536,779 | CID 21,536,783 | CID 21,544,015 | CID 21,584,652 | CID 21,584,653 | CID 21,612,055 |
| CID 21,624,658 | CID 21,646,552 | CID 21,715,449 | CID 21,715,463 | CID 21,716,090 | CID 21,732,898 |
| CID 21,748,563 | CID 21,752,591 | CID 21,760,500 | CID 21,760,511 | CID 21,798,788 | CID 21,808,506 |
| CID 21,845,204 | CID 21,880,523 | CID 21,881,274 | CID 21,881,815 | CID 21,881,945 | CID 21,888,690 |
| CID 21,888,691 | CID 21,910,092 | CID 21,910,161 | CID 21,910,167 | CID 21,910,205 | CID 21,910,220 |
| CID 21,910,731 | CID 21,924,641 | CID 21,930,582 | CID 21,944,883 | CID 21,960,621 | CID 21,971,281 |
| CID 21,998,298 | CID 21,998,316 | CID 22,023,671 | CID 22,044,206 | CID 22,066,493 | CID 22,138,168 |
| CID 22,138,169 | CID 22,141,747 | CID 22,184,136 | CID 22,234,755 | CID 22,237,127 | CID 22,237,128 |
| CID 22,238,516 | CID 22,256,074 | CID 22,323,793 | CID 22,336,054 | CID 22,342,054 | CID 22,342,102 |
| CID 22,342,120 | CID 22,342,127 | CID 22,342,155 | CID 22,342,157 | CID 22,342,208 | CID 22,342,232 |
| CID 22,342,254 | CID 22,342,255 | CID 22,342,257 | CID 22,342,266 | CID 22,342,270 | CID 22,439,115 |
| CID 22,450,932 | CID 22,479,650 | CID 22,481,576 | CID 22,495,054 | CID 22,572,164 | CID 22,596,865 |
| CID 22,603,871 | CID 22,625,575 | CID 22,693,028 | CID 22,693,029 | CID 22,708,915 | CID 22,718,963 |
| CID 22,743,281 | CID 22,743,405 | CID 22,795,593 | CID 22,804,163 | CID 22,874,163 | CID 22,898,390 |
| CID 22,988,986 | CID 22,988,999 | CID 22,989,035 | CID 23,019,778 | CID 23,034,996 | CID 23,054,262 |
| CID 23,079,060 | CID 23,132,927 | CID 23,132,935 | CID 23,132,951 | CID 23,132,964 | CID 23,132,971 |
| CID 23,132,989 | CID 23,133,025 | CID 23,133,075 | CID 23,133,081 | CID 23,133,107 | CID 23,133,123 |
| CID 23,133,145 | CID 23,133,154 | CID 23,133,219 | CID 23,175,350 | CID 23,182,090 | CID 23,194,872 |
| CID 23,223,696 | CID 23,234,551 | CID 23,234,552 | CID 23,234,553 | CID 23,236,023 | CID 23,236,101 |
| CID 23,236,101 | CID 23,237,635 | CID 23,237,636 | CID 23,237,637 | CID 23,265,870 | CID 23,265,678 |
| CID 23,265,893 | CID 23,265,971 | CID 23,266,003 | CID 23,266,004 | CID 23,267,272 | CID 23,267,743 |
| CID 23,268,153 | CID 23,268,251 | CID 23,268,362 | CID 23,268,493 | CID 23,268,536 | CID 23,268,879 |
| CID 23,268,994 | CID 23,269,122 | CID 23,269,200 | CID 23,269,208 | CID 23,269,478 | CID 23,269,479 |
| CID 23,269,535 | CID 23,293,286 | CID 23,293,287 | CID 23,293,288 | CID 23,297,479 | CID 23,317,906 |
| CID 23,339,742 | CID 23,371,233 | CID 23,414,025 | CID 23,422,913 | CID 23,422,919 | CID 23,422,971 |
| CID 23,423,076 | CID 23,423,302 | CID 23,423,303 | CID 23,423,304 | CID 23,423,305 | CID 23,423,306 |
| CID 23,423,307 | CID 23,423,308 | CID 23,423,309 | CID 23,423,310 | CID 23,423,311 | CID 23,423,384 |
| CID 23,423,385 | CID 23,462,625 | CID 23,509,052 | CID 23,615,241 | CID 23,615,242 | CID 23,615,614 |
| CID 23,615,615 | CID 23,615,616 | CID 23,616,635 | CID 23,617,006 | CID 23,617,095 | CID 23,617,096 |
| CID 23,617,097 | CID 23,617,098 | CID 23,617,099 | CID 23,617,100 | CID 23,617,156 | CID 23,617,157 |
| CID 23,617,770 | CID 23,618,480 | CID 23,618,483 | CID 23,618,484 | CID 23,618,493 | CID 23,618,494 |
| CID 23,618,537 | CID 23,618,592 | CID 23,618,632 | CID 23,618,633 | CID 23,618,636 | CID 23,618,697 |
| CID 23,618,805 | CID 23,618,840 | CID 23,618,850 | CID 23,618,851 | CID 23,618,852 | CID 23,618,853 |
| CID 23,618,854 | CID 23,618,910 | CID 23,618,963 | CID 23,619,068 | CID 23,619,958 | CID 23,621,942 |
| CID 23,621,970 | CID 23,622,126 | CID 23,622,688 | CID 23,622,689 | CID 23,623,716 | CID 23,623,741 |
| CID 23,623,742 | CID 23,639,731 | CID 23,644,513 | CID 23,664,719 | CID 23,665,572 | CID 23,665,811 |
| CID 23,667,268 | CID 23,667,269 | CID 23,668,632 | CID 23,670,523 | CID 23,670,848 | CID 23,671,187 |
| CID 23,673,647 | CID 23,673,838 | CID 23,675,353 | CID 23,675,510 | CID 23,675,762 | CID 23,676,536 |
| CID 23,677,060 | CID 23,678,030 | CID 23,678,031 | CID 23,678,498 | CID 23,678,861 | CID 23,679,054 |
| CID 23,679,059 | CID 23,679,827 | CID 23,681,930 | CID 23,684,591 | CID 23,664,831 | CID 23,684,835 |
| CID 23,687,156 | CID 23,688,636 | CID 23,689,040 | CID 23,690,436 | CID 23,692,071 | CID 23,695,998 |
| CID 23,699,679 | CID 23,701,920 | CID 23,702,397 | CID 23,704,689 | CID 23,704,946 | CID 23,705,691 |
| CID 23,711,769 | CID 23,712,046 | CID 23,712,047 | CID 23,714,612 | CID 23,714,613 | CID 23,714,614 |
| CID 23,714,615 | CID 23,714,616 | CID 23,719,509 | CID 23,719,536 | CID 23,725,018 | CID 24,180,936 |
| CID 24,181,706 | CID 24,181,707 | CID 24,181,708 | CID 24,181,711 | CID 24,181,835 | CID 24,181,841 |
| CID 24,181,927 | CID 24,182,103 | CID 24,182,110 | CID 24,182,111 | CID 24,182,114 | CID 24,182,116 |
| CID 24,182,325 | CID 24,182,334 | CID 24,182,367 | CID 24,183,873 | CID 24,163,928 | CID 24,184,262 |
| CID 24,184,949 | CID 24,185,838 | CID 24,188,024 | CID 24,188,025 | CID 24,190,450 | CID 24,192,474 |
| CID 24,192,485 | CID 24,196,464 | CID 24,196,465 | CID 24,198,998 | CID 24,319,283 | CID 24,833,845 |
| CID 24,833,846 | CID 24,833,847 | CID 24,833,848 | CID 24,833,849 | CID 24,833,850 | CID 24,833,851 |
| CID 24,833,852 | CID 24,833,853 | CID 24,833,854 | CID 24,833,855 | CID 24,833,856 | CID 24,833,857 |
| CID 24,833,858 | CID 24,833,859 | CID 24,833,860 | CID 24,833,861 | CID 24,833,862 | CID 24,833,863 |
| CID 24,833,864 | CID 24,833,865 | CID 24,833,866 | CID 24,833,867 | CID 24,833,868 | CID 24,833,869 |
| CID 24,833,870 | CID 24,833,871 | CID 24,833,872 | CID 24,833,873 | CID 24,833,874 | CID 24,833,875 |
| CID 24,833,876 | CID 24,833,877 | CID 24,833,878 | CID 24,833,884 | CID 24,834,489 | CID 24,834,490 |
| CID 24,834,491 | CID 24,834,492 | CID 24,834,493 | CID 24,834,494 | CID 24,834,495 | CID 24,834,496 |
| CID 24,834,497 | CID 24,834,498 | CID 24,834,499 | CID 24,834,500 | CID 24,834,501 | CID 24,834,502 |
| CID 24,834,503 | CID 24,834,504 | CID 24,834,505 | CID 24,834,506 | CID 24,834,507 | CID 24,835,071 |
| CID 24,836,124 | CID 24,838,478 | CID 24,838,850 | CID 24,838,851 | CID 24,833,852 | CID 24,840,406 |
| CID 24,847,717 | CID 24,847,718 | CID 24,884,165 | CID 25,021,277 | CID 25,021,716 | CID 25,022,094 |
| CID 25,022,139 | CID 25,059,847 | CID 25,147,438 | CID 25,201,247 | CID 25,202,100 | CID 25,215,427 |
| CID 25,258,926 | CID 42,616,376 | CID 42,626,516 | CID 42,626,649 | CID 42,626,869 | CID 43,834,996 |
| CID 44,134,551 | CID 44,134,552 | CID 44,135,769 | CID 44,135,770 | CID 44,135,830 | CID 44,135,912 |
| CID 44,144,455 | CID 44,146,058 | CID 44,146,486 | CID 44,146,567 | CID 44,146,730 | CID 44,146,916 |
| CID 44,147,059 | CID 44,148,502 | CID 44,150,404 | CID 44,150,532 | CID 44,150,701 | CID 44,150,948 |
| CID 44,151,798 | CID 44,152,287 | CID 44,152,840 | CID 44,154,459 | CID 44,154,483 | CID 44,154,512 |
| CID 44,154,561 | CID 44,154,582 | CID 44,154,630 | CID 44,154,866 | CID 44,154,920 | CID 44,321,327 |
| CID 44,369,386 | CID 44,370,063 | CID 44,558,881 | CID 44,558,882 | CID 44,593,649 | CID 44,603,086 |
| CID 44,630,230 | CID 44,720,830 | CID 44,721,082 | CID 44,721,083 | CID 44,725,754 | CID 45,048,798 |
| CID 45,048,892 | CID 45,050,564 | CID 45,051,682 | CID 45,266,914 | CID 45,479,758 | CID 46,224,551 |
| CID 46,224,552 | CID 46,224,593 | CID 46,875,030 | CID 46,936,754 | CID 49,789,061 | CID 50,896,984 |
| CID 50,910,470 | CID 50,920,338 | CID 50,921,205 | CID 50,921,206 | CID 50,930,983 | CID 50,931,253 |
| CID 50,931,674 | CID 50,932,276 | CID 50,933,749 | CID 50,989,345 | CID 50,989,963 | CID 51,000,486 |
| CID 53,339,029 | CID 53,384,454 | CID 53,393,493 | CID 53,393,598 | CID 53,400,626 | CID 53,405,325 |
| CID 53,428,512 | CID 53,439,260 | CID 53,462,054 | CID 53,463,799 | CID 53,471,871 | CID 53,471,920 |
| CID 54,598,359 | CID 54,599,412 | CID 54,601,637 | CID 54,602,507 | CID 54,602,768 | CID 54,602,880 |
| CID 54,603,132 | CID 54,603,241 | CID 54,603,485 | CID 54,603,489 | CID 54,603,573 | CID 54,603,865 |
| CID 54,604,418 | CID 54,605,078 | CID 54,605,477 | CID 54,605,894 | CID 54,606,603 | CID 54,607,273 |
| CID 54,610,700 | CID 54,611,643 | CID 54,612,078 | CID 54,612,079 | CID 56,841,614 | CID 56,841,734 |
| CID 56,842,095 | CID 56,842,269 | CID 56,843,347 | CID 56,843,418 | CID 56,843,823 | CID 56,925,092 |
| CID 56,955,921 | CID 56,955,922 | CID 56,955,923 | CID 56,955,924 | CID 57,347,038 | CID 57,347,038 |
| CID 57,347,159 | CID 57,347,436 | CID 57,348,041 | CID 57,348,043 | CID 57,348,098 | CID 57,348,555 |
| CID 57,348,941 | CID 57,349,419 | CID 57,349,472 | CID 57,349,580 | CID 57,349,581 | CID 57,350,047 |
| CID 57,350,047 | CID 57,350,641 | CID 57,350,785 | CID 57,350,803 | CID 57,351,175 | CID 57,351,442 |
| CID 57,351,614 | CID 57,351,668 | CID 57,351,752 | CID 57,352,657 | CID 57,353,494 | CID 57,358,064 |
| CID 57,358,206 | CID 57,358,236 | CID 57,359,122 | CID 57,359,123 | CID 57,365,615 | CID 57,386,366 |
| CID 57,397,090 | CID 57,418,239 | CID 57,422,000 | CID 57,422,093 | CID 57,448,801 | CID 57,448,802 |
| CID 57,448,899 | CID 57,449,070 | CID 57,449,169 | CID 57,449,394 | CID 57,450,418 | CID 57,451,424 |
| CID 57,459,958 | CID 57,464,763 | CID 57,464,821 | CID 57,464,908 | CID 57,464,921 | CID 57,465,286 |
| CID 57,466,691 | CID 57,466,703 | CID 57,469,302 | CID 57,469,420 | CID 57,485,235 | CID 57,485,401 |
| CID 57,490,558 | CID 57,507,417 | CID 57,507,848 | CID 57,507,876 | CID 57,514,279 | CID 57,514,722 |
| CID 57,517,132 | CID 57,517,138 | CID 57,517,933 | CID 57,763,375 | CID 58,165,711 | CID 58,165,712 |
| CID 56,165,713 | CID 58,165,714 | CID 58,165,715 | CID 58,165,716 | CID 58,165,717 | CID 58,165,718 |
| CID 58,165,720 | CID 58,165,721 | CID 58,165,723 | CID 58,165,724 | CID 58,165,726 | CID 58,165,727 |
| CID 58,165,730 | CID 58,165,731 | CID 58,165,733 | CID 58,165,734 | CID 58,165,735 | CID 58,165,736 |
| CID 58,165,737 | CID 58,165,738 | CID 58,302,438 | CID 58,671,705 | CID 58,671,705 | CID 58,729,368 |
| CID 58,729,369 | CID 58,729,370 | CID 58,729,371 | CID 58,729,372 | CID 58,729,373 | CID 58,729,374 |
| CID 58,729,375 | CID 58,729,377 | CID 58,729,379 | CID 58,729,380 | CID 58,729,381 | CID 58,729,382 |
| CID 58,729,383 | CID 58,729,384 | CID 58,729,387 | CID 58,729,388 | CID 58,729,389 | CID 58,729,390 |
| CID 58,729,391 | CID 58,729,392 | CID 58,729,333 | CID 58,729,394 | CID 58,729,397 | CID 58,729,398 |
| CID 58,729,399 | CID 58,886,963 | CID 59,263,990 | CID 59,467,531 | CID 59,467,532 | CID 59,467,533 |
| CID 59,467,534 | CID 59,467,536 | CID 59,467,537 | CID 59,467,538 | CID 59,467,539 | CID 59,467,540 |
| CID 59,467,542 | CID 59,467,543 | CID 59,467,545 | CID 59,467,546 | CID 59,467,549 | CID 59,467,550 |
| CID 59,467,552 | CID 59,467,553 | CID 59,467,554 | CID 59,467,556 | CID 59,467,557 | CID 59,865,558 |
| CID 59,940,674 | CID 59,940,675 | CID 60,036,552 | CID 60,036,553 | CID 60,165,464 | CID 60,208,620 |
| CID 60,208,621 | CID 60,208,652 | CID 66,545,852 | CID 70,297,382 | CID 70,675,072 | CID 70,675,073 |
| CID 70,700,067 | CID 70,702,302 | CID 71,299,673 | CID 71,300,916 | CID 71,308,157 | CID 71,313,487 |
| CID 71,313,488 | CID 71,317,296 | CID 71,319,306 | CID 71,319,307 | CID 71,319,865 | CID 71,327,504 |
| CID 71,328,225 | CID 71,330,499 | CID 71,331,141 | CID 71,332,057 | CID 71,334,109 | CID 71,335,003 |
| CID 71,335,466 | CID 71,335,471 | CID 71,336,056 | CID 71,337,069 | CID 71,338,314 | CID 71,339,210 |
| CID 71,339,611 | CID 71,339,840 | CID 71,340,597 | CID 71,342,472 | CID 71,348,368 | CID 71,348,670 |
| CID 71,354,187 | CID 71,357,095 | CID 71,359,057 | CID 71,360,254 | CID 71,361,127 | CID 71,361,431 |
| CID 71,363,441 | CID 71,363,442 | CID 71,363,446 | CID 71,363,802 | CID 71,364,348 | CID 71,364,378 |
| CID 71,365,365 | CID 71,366,353 | CID 71,367,154 | CID 71,367,770 | CID 71,367,894 | CID 71,367,895 |
| CID 71,368,197 | CID 71,368,508 | CID 71,370,280 | CID 71,371,122 | CID 71,371,820 | CID 71,372,565 |
| CID 71,372,674 | CID 71,380,037 | CID 71,380,573 | CID 71,381,041 | CID 71,381,042 | CID 71,382,184 |
| CID 71,382,439 | CID 71,382,445 | CID 71,385,726 | CID 71,385,727 | CID 71,387,140 | CID 71,387,143 |
| CID 71,387,509 | CID 71,334,297 | CID 71,394,403 | CID 71,394,404 | CID 71,397,369 | CID 71,398,232 |
| CID 71,399,333 | CID 71,401,268 | CID 71,401,326 | CID 71,401,532 | CID 71,401,921 | CID 71,405,075 |
| CID 71,408,659 | CID 71,409,840 | CID 71,410,005 | CID 71,410,222 | CID 71,410,223 | CID 71,421,391 |
| CID 71,430,951 | CID 71,430,977 | CID 71,431,024 | CID 71,431,066 | CID 71,436,586 | CID 71,437,827 |
| CID 71,444,769 | CID 71,444,988 | CID 71,446,559 | CID 71,446,674 | CID 71,449,004 | CID 71,459,681 |
| CID 71,479,335 | CID 71,487,561 | CID 71,586,823 | CID 71,586,865 | CID 71,586,922 | CID 71,710,894 |
| CID 71,774,626 | CID 72,941,484 | CID 73,415,795 | CID 73,427,316 | CID 73,427,319 | CID 73,427,321 |
| CID 73,555,192 | CID 73,555,265 | CID 73,556,100 | CID 73,894,235 | CID 73,894,259 | CID 73,894,260 |
| CID 73,894,261 | CID 73,894,262 | CID 73,894,263 | CID 73,894,264 | CID 73,894,265 | CID 73,894,266 |
| CID 73,894,267 | CID 73,894,268 | CID 73,894,269 | CID 73,894,270 | CID 73,894,271 | CID 73,894,272 |
| CID 73,894,273 | CID 73,894,274 | CID 73,894,275 | CID 73,894,276 | CID 73,894,277 | CID 73,894,278 |
| CID 73,897,259 | CID 73,951,871 | CID 73,994,974 | CID 74,764,745 | CID 74,765,628 | CID 74,765,632 |
| CID 74,765,637 | CID 75,124,214 | CID 76,198,490 | CID 76,419,686 | CID 76,957,520 | CID 76,958,471 |
| CID 78,063,565 | CID 78,064,980 | CID 78,066,137 | CID 78,067,178 | CID 78,067,215 | CID 78,067,216 |
| CID 82,030,612 | CID 82,030,683 | CID 82,316,598 | CID 82,316,626 | CID 82,316,722 | CID 82,318,949 |
| CID 82,363,720 | CID 82,363,766 | CID 83,768,323 | CID 83,773,659 | CID 84,226,900 | CID 84,228,930 |
| CID 84,228,952 | CID 84,229,512 | CID 84,229,564 | CID 84,230,181 | CID 84,230,317 | CID 84,230,491 |
| CID 84,230,496 | CID 84,230,505 | CID 84,232,747 | CID 84,251,793 | CID 84,271,289 | CID 84,271,519 |
| CID 84,271,720 | CID 84,273,649 | CID 84,293,452 | CID 84,313,624 | CID 84,317,251 | CID 84,317,410 |
| CID 84,317,532 | CID 84,317,592 | CID 84,317,617 | CID 84,317,748 | CID 84,317,859 | CID 84,317,938 |
| CID 84,318,269 | CID 84,318,352 | CID 84,318,548 | CID 84,318,697 | CID 84,319,320 | CID 84,319,537 |
| CID 84,319,696 | CID 84,319,949 | CID 84,320,164 | CID 84,320,412 | CID 84,320,471 | CID 84,321,900 |
| CID 84,321,961 | CID 84,322,271 | CID 84,322,555 | CID 84,322,803 | CID 84,323,164 | CID 84,323,196 |
| CID 84,323,348 | CID 64,323,444 | CID 84,331,163 | CID 84,331,188 | CID 85,671,405 | CID 85,677,163 |
| CID 85,683,074 | CID 85,764,410 | CID 85,844,805 | CID 85,858,957 | CID 85,901,868 | CID 85,970,572 |
| CID 86,171,938 | CID 86,181,093 | CID 86,278,207 | CID 86,341,919 | CID 86,341,922 | CID 86,618,681 |
| CID 86,618,683 | CID 86,618,685 | CID 86,743,524 | CID 86,743,525 | CID 86,745,607 | CID 87,058,047 |
| CID 87,059,370 | CID 87,072,965 | CID 87,073,127 | CID 87,073,128 | CID 87,073,129 | CID 87,073,143 |
| CID 87,073,608 | CID 87,086,209 | CID 87,086,388 | CID 87,090,261 | CID 87,106,887 | CID 87,110,451 |
| CID 87,131,993 | CID 87,131,994 | CID 87,131,995 | CID 87,131,996 | CID 87,131,997 | CID 87,132,131 |
| CID 87,132,524 | CID 87,132,525 | CID 87,132,526 | CID 87,144,976 | CID 87,165,815 | CID 87,172,195 |
| CID 87,172,196 | CID 87,195,630 | CID 87,205,220 | CID 87,205,221 | CID 87,205,976 | CID 87,233,402 |
| CID 87,233,403 | CID 87,234,663 | CID 87,234,712 | CID 87,238,627 | CID 87,238,628 | CID 87,238,633 |
| CID 87,240,211 | CID 87,251,088 | CID 87,255,861 | CID 87,255,862 | CID 87,255,870 | CID 87,255,875 |
| CID 87,255,880 | CID 87,255,881 | CID 87,269,476 | CID 87,269,477 | CID 87,314,093 | CID 87,318,793 |
| CID 87,330,514 | CID 87,344,746 | CID 87,350,680 | CID 87,359,256 | CID 87,438,458 | CID 87,438,459 |
| CID 87,438,573 | CID 87,438,574 | CID 87,438,777 | CID 87,438,779 | CID 87,458,202 | CID 87,458,203 |
| CID 87,458,204 | CID 87,458,727 | CID 87,472,400 | CID 87,472,523 | CID 87,472,811 | CID 87,472,864 |
| CID 87,473,748 | CID 87,474,375 | CID 87,474,815 | CID 87,475,704 | CID 87,476,010 | CID 87,476,169 |
| CID 87,476,500 | CID 87,476,559 | CID 87,476,847 | CID 87,477,538 | CID 87,477,549 | CID 87,478,368 |
| CID 87,491,392 | CID 87,491,700 | CID 87,492,961 | CID 87,499,136 | CID 87,501,741 | CID 87,505,017 |
| CID 87,510,533 | CID 87,514,724 | CID 87,514,827 | CID 87,562,629 | CID 87,603,632 | CID 87,606,799 |
| CID 87,606,893 | CID 87,606,894 | CID 87,606,895 | CID 87,640,134 | CID 87,656,152 | CID 87,657,080 |
| CID 87,657,747 | CID 87,662,947 | CID 87,671,297 | CID 87,671,298 | CID 87,671,912 | CID 87,699,275 |
| CID 87,699,545 | CID 87,711,479 | CID 87,721,746 | CID 87,735,226 | CID 87,738,731 | CID 87,738,732 |
| CID 87,739,172 | CID 87,743,549 | CID 87,755,255 | CID 87,756,357 | CID 87,756,359 | CID 87,762,269 |
| CID 87,763,110 | CID 87,764,999 | CID 87,765,000 | CID 87,770,098 | CID 87,770,571 | CID 87,805,032 |
| CID 87,805,033 | CID 87,841,306 | CID 87,841,308 | CID 87,852,890 | CID 87,934,277 | CID 87,935,205 |
| CID 87,945,130 | CID 87,967,607 | CID 87,979,929 | CID 88,005,023 | CID 88,006,712 | CID 88,027,044 |
| CID 88,027,224 | CID 88,027,225 | CID 88,027,247 | CID 88,027,249 | CID 88,027,349 | CID 88,027,898 |
| CID 88,028,174 | CID 88,028,175 | CID 88,028,382 | CID 88,047,523 | CID 88,048,162 | CID 88,048,239 |
| CID 88,048,240 | CID 88,048,687 | CID 88,048,689 | CID 88,048,692 | CID 88,048,693 | CID 88,085,429 |
| CID 88,085,430 | CID 88,085,672 | CID 88,085,673 | CID 88,085,737 | CID 88,085,790 | CID 86,085,882 |
| CID 88,086,427 | CID 88,086,990 | CID 88,090,309 | CID 88,103,022 | CID 88,112,731 | CID 88,112,732 |
| CID 88,113,921 | CID 88,162,367 | CID 88,162,370 | CID 88,163,324 | CID 88,171,188 | CID 88,195,082 |
| CID 88,195,459 | CID 88,199,335 | CID 88,202,050 | CID 88,223,011 | CID 88,234,680 | CID 88,237,233 |
| CID 88,247,261 | CID 88,247,263 | CID 88,282,576 | CID 88,282,578 | CID 88,288,218 | CID 88,312,532 |
| CID 88,314,934 | CID 88,314,935 | CID 88,327,898 | CID 88,328,145 | CID 88,339,646 | CID 88,339,769 |
| CID 88,339,770 | CID 88,342,230 | CID 88,360,632 | CID 88,367,464 | CID 88,367,467 | CID 88,370,019 |
| CID 88,371,460 | CID 88,371,540 | CID 88,383,641 | CID 88,384,192 | CID 88,402,848 | CID 88,404,033 |
| CID 88,414,089 | CID 88,421,853 | CID 88,421,854 | CID 88,423,689 | CID 88,423,693 | CID 88,423,694 |
| CID 88,423,747 | CID 88,424,044 | CID 88,424,045 | CID 88,424,429 | CID 88,440,204 | CID 88,440,205 |
| CID 88,446,062 | CID 88,453,441 | CID 88,454,696 | CID 88,458,282 | CID 88,463,436 | CID 88,470,126 |
| CID 88,470,129 | CID 88,473,659 | CID 88,473,660 | CID 88,481,422 | CID 88,488,676 | CID 88,510,887 |
| CID 88,602,316 | CID 88,610,141 | CID 88,610,142 | CID 88,611,072 | CID 88,613,537 | CID 88,613,538 |
| CID 88,613,539 | CID 88,633,323 | CID 88,633,324 | CID 88,636,491 | CID 88,640,649 | CID 88,641,981 |
| CID 88,641,982 | CID 88,642,858 | CID 88,642,860 | CID 88,670,623 | CID 88,680,217 | CID 88,680,218 |
| CID 88,681,635 | CID 88,681,637 | CID 88,683,805 | CID 88,688,468 | CID 88,709,942 | CID 88,714,945 |
| CID 88,717,455 | CID 88,728,174 | CID 88,728,830 | CID 88,736,668 | CID 88,738,483 | CID 88,740,953 |
| CID 88,741,022 | CID 88,741,024 | CID 88,745,619 | CID 88,746,533 | CID 88,756,177 | CID 88,762,995 |
| CID 88,763,319 | CID 88,786,396 | CID 88,791,224 | CID 88,807,784 | CID 88,807,786 | CID 88,827,124 |
| CID 88,828,033 | CID 88,836,686 | CID 88,847,453 | CID 88,848,338 | CID 89,422,442 | CID 89,573,835 |
| CID 89,972,012 | CID 89,977,504 | CID 90,021,411 | CID 90,028,968 | CID 90,471,423 | CID 90,472,865 |
| CID 90,472,953 | CID 90,472,954 | CID 90,476,144 | CID 90,476,459 | CID 90,478,957 | CID 90,479,639 |
| CID 90,657,387 | CID 90,657,388 | CID 90,661,369 | CID 90,661,495 | CID 91,617,718 | CID 91,618,135 |
| CID 91,809,010 | CID 91,809,011 | CID 91,872,575 | CID 91,872,576 | CID 91,873,289 | CID 91,885,296 |
| CID 91,886,247 | CID 91,886,305 | CID 91,886,306 | CID 91,886,379 | CID 91,977,524 | CID 91,977,525 |
| CID 91,979,699 | CID 91,979,700 | CID 91,979,701 | CID 91,979,702 | CID 91,982,311 | CID 91,990,907 |
| CID 91,990,908 | CID 91,996,625 | CID 91,997,872 | CID 91,997,873 | CID 91,998,119 | CID 91,998,120 |
| CID 91,998,157 | CID 91,998,324 | CID 92,003,746 | CID 92,003,947 | CID 92,004,943 | CID 92,006,598 |
| CID 92,006,641 | CID 92,006,915 | CID 92,006,992 | CID 92,006,993 | CID 92,006,994 | CID 92,006,995 |
| CID 92,006,996 | CID 92,006,997 | CID 92,006,998 | CID 92,006,999 | CID 92,007,000 | CID 92,007,001 |
| CID 92,007,002 | CID 92,007,003 | CID 92,007,004 | CID 92,007,005 | CID 92,007,006 | CID 92,007,007 |
| CID 92,007,008 | CID 92,007,009 | CID 92,007,010 | CID 92,007,011 | CID 92,007,017 | CID 32,007,451 |
| CID 92,008,067 | CID 92,008,243 | CID 92,008,621 | CID 92,009,736 | CID 92,010,994 | CID 92,010,995 |
| CID 92,010,996 | CID 92,010,997 | CID 92,013,326 | CID 92,022,904 | CID 92,024,946 | CID 92,024,947 |
| CID 92,025,093 | CID 92,025,094 | CID 92,025,095 | CID 92,025,096 | CID 92,025,097 | CID 92,025,098 |
| CID 92,025,099 | CID 92,025,100 | CID 92,025,114 | CID 92,025,115 | CID 92,025,116 | CID 92,025,117 |
| CID 92,025,118 | CID 92,025,119 | CID 92,025,120 | CID 32,025,121 | CID 92,025,124 | CID 92,026,484 |
| CID 92,026,485 | CID 92,026,502 | CID 92,026,503 | CID 92,026,560 | CID 92,026,596 | CID 92,026,616 |
| CID 92,027,384 | CID 92,027,405 | CID 92,027,424 | CID 92,027,436 | CID 92,028,137 | CID 92,028,138 |
| CID 92,028,742 | CID 92,028,792 | CID 92,028,793 | CID 92,028,841 | CID 92,028,912 | CID 92,038,599 |
| CID 92,042,585 | CID 92,043,501 | CID 92,043,987 | CID 92,131,721 | CID 92,135,763 | CID 100,919,003 |
| CID 100,931,586 | CID 100,934,184 | CID 100,952,680 | CID 100,963,338 | CID 100,968,929 | CID 100,986,762 |
| CID 100,986,764 | CID 100,993,010 | CID 100,999,029 | CID 101,005,691 | CID 101,006,949 | CID 101,009,191 |
| CID 101,009,416 | CID 101,015,656 | CID 101,025,050 | CID 101,025,051 | CID 101,046,544 | CID 101,052,365 |
| CID 101,053,279 | CID 101,053,279 | CID 101,053,279 | CID 101,053,280 | CID 101,064,349 | CID 101,071,247 |
| CID 101,093,527 | CID 101,100,195 | CID 101,100,195 | CID 101,116,060 | CID 101,118,643 | CID 101,118,644 |
| CID 101,122,537 | CID 101,127,381 | CID 101,136,872 | CID 101,137,899 | CID 101,139,985 | CID 101,139,986 |
| CID 101,200,916 | CID 101,211,580 | CID 101,230,437 | CID 101,230,438 | CID 101,230,439 | CID 101,243,708 |
| CID 101,255,615 | CID 101,257,613 | CID 101,261,664 | CID 101,288,890 | CID 101,298,727 | CID 101,318,322 |
| CID 101,340,809 | CID 101,348,281 | CID 101,354,488 | CID 101,354,489 | CID 101,357,168 | CID 101,357,169 |
| CID 101,357,594 | CID 101,357,595 | CID 101,377,149 | CID 101,397,671 | CID 101,409,658 | CID 101,415,375 |
| CID 101,415,376 | CID 101,440,809 | CID 101,458,324 | CID 101,458,325 | CID 101,458,327 | CID 101,478,419 |
| CID 101,495,974 | CID 101,504,033 | CID 101,522,049 | CID 101,533,857 | CID 101,564,671 | CID 101,586,483 |
| CID 101,599,593 | CID 101,599,594 | CID 101,611,448 | CID 101,614,891 | CID 101,616,131 | CID 101,625,829 |
| CID 101,625,830 | CID 101,625,831 | CID 101,625,832 | CID 101,635,676 | CID 101,652,314 | CID 101,652,556 |
| CID 101,652,557 | CID 101,652,558 | CID 101,652,559 | CID 101,664,275 | CID 101,664,531 | CID 101,664,931 |
| CID 101,667,977 | CID 101,674,978 | CID 101,674,979 | CID 101,674,980 | CID 101,674,981 | CID 101,674,982 |
| CID 101,674,983 | CID 101,686,450 | CID 101,689,360 | CID 101,691,534 | CID 101,691,535 | CID 101,691,536 |
| CID 101,691,537 | CID 101,691,538 | CID 101,691,539 | CID 101,691,632 | CID 101,691,633 | CID 101,691,634 |
| CID 101,691,635 | CID 101,691,636 | CID 101,691,637 | CID 101,699,244 | CID 101,708,462 | CID 101,708,463 |
| CID 101,715,578 | CID 101,716,440 | CID 101,723,246 | CID 101,786,483 | CID 101,786,862 | CID 101,786,863 |
| CID 101,805,070 | CID 101,846,346 | CID 101,847,764 | CID 101,883,650 | CID 101,889,836 | CID 101,889,839 |
| CID 101,895,131 | CID 101,895,132 | CID 101,913,583 | CID 101,916,334 | CID 101,928,003 | CID 101,929,420 |
| CID 101,930,742 | CID 101,946,400 | CID 101,970,427 | CID 101,972,020 | CID 101,978,331 | CID 101,978,342 |
| CID 101,978,385 | CID 101,988,596 | CID 101,993,105 | CID 101,996,063 | CID 102,012,028 | CID 102,013,216 |
| CID 102,013,636 | CID 102,039,635 | CID 102,054,416 | CID 102,072,502 | CID 102,075,650 | CID 102,129,552 |
| CID 102,137,137 | CID 102,160,647 | CID 102,171,729 | CID 102,215,006 | CID 102,239,767 | CID 102,248,352 |
| CID 102,261,461 | CID 102,279,703 | CID 102,279,704 | CID 102,279,705 | CID 102,279,706 | CID 102,279,707 |
| CID 102,279,708 | CID 102,279,709 | CID 102,283,040 | CID 102,283,045 | CID 102,283,046 | CID 102,326,471 |
| CID 102,393,511 | CID 102,409,673 | CID 102,413,161 | CID 102,416,167 | CID 102,438,080 | CID 102,439,215 |
| CID 102,533,224 | CID 102,595,004 | CID 102,595,005 | CID 102,601,898 | CID 110,210,780 | CID 110,210,781 |
| CID 110,431,250 | CID 110,499,257 | CID 110,499,461 | CID 110,499,630 | CID 110,499,730 | CID 110,499,847 |
| CID 110,500,056 | CID 110,500,265 | CID 110,500,474 | CID 110,500,682 | CID 110,500,891 | CID 110,501,100 |
| CID 110,501,309 | CID 110,501,518 | CID 110,501,727 | CID 110,501,936 | CID 110,502,145 | CID 110,502,354 |
| CID 112,501,307 | CID 112,501,308 | CID 112,501,309 | CID 112,501,310 | CID 112,501,311 | CID 112,501,312 |
| CID 112,501,313 | CID 112,501,314 | CID 112,501,315 | CID 112,501,316 | CID 112,501,317 | CID 112,501,318 |
| CID 112,501,319 | CID 112,501,320 | CID 112,501,321 | CID 112,501,322 | CID 112,501,323 | CID 112,501,324 |
| CID 112,501,325 | CID 112,501,326 | CID 112,501,327 | CID 112,501,328 | CID 112,501,329 | CID 112,501,330 |
| CID 112,501,331 | CID 112,501,332 | CID 112,501,333 | CID 112,501,334 | CID 112,501,335 | CID 112,501,336 |
| CID 112,501,337 | CID 112,501,338 | CID 112,501,339 | CID 117,059,131 | CID 118,984,370 | CID 118,985,489 |
| CID 118,985,493 | CID 121,233,249 | CID 121,233,660 | CID 121,233,703 | CID 122,201,230 | CID 122,228,415 |
| CID 122,363,780 | CID 122,363,783 | CID 122,403,017 | CID 123,894,049 | CID 124,204,125 | CID 129,318,123 |
| CID 129,318,137 | CID 129,627,698 | CID 129,629,450 | CID 129,629,459 | CID 129,629,462 | CID 129,629,468 |
| CID 129,629,516 | CID 129,629,522 | CID 129,629,530 | CID 129,630,137 | CID 129,631,095 | CID 129,632,050 |
| CID 129,632,468 | CID 129,632,826 | CID 129,633,897 | CID 129,636,342 | CID 129,636,343 | CID 129,636,810 |
| CID 129,636,925 | CID 129,641,160 | CID 129,648,732 | CID 129,652,278 | CID 129,653,851 | CID 129,656,420 |
| CID 129,656,532 | CID 129,657,293 | CID 129,657,294 | CID 129,657,295 | CID 129,657,300 | CID 129,657,304 |
| CID 129,657,312 | CID 129,657,315 | CID 129,657,324 | CID 129,657,326 | CID 129,657,327 | CID 129,657,329 |
| CID 129,657,337 | CID 129,657,353 | CID 129,664,230 | CID 129,664,232 | CID 129,664,293 | CID 129,666,899 |
| CID 129,667,149 | CID 129,667,246 | CID 129,667,772 | CID 129,668,316 | CID 129,669,884 | CID 129,672,174 |
| CID 129,672,290 | CID 129,672,646 | CID 129,673,528 | CID 129,673,557 | CID 129,674,090 | CID 129,675,341 |
| CID 129,676,928 | CID 129,677,374 | CID 129,677,629 | CID 129,677,673 | CID 129,677,709 | CID 129,678,442 |
| CID 129,680,161 | CID 129,680,629 | CID 129,686,725 | CID 129,687,036 | CID 129,687,164 | CID 129,687,794 |
| CID 129,689,537 | CID 129,690,605 | CID 129,690,606 | CID 129,693,550 | CID 129,693,600 | CID 129,695,429 |
| CID 129,699,101 | CID 129,699,962 | CID 129,703,922 | CID 129,704,872 | CID 129,704,873 | CID 129,704,991 |
| CID 129,705,080 | CID 129,710,768 | CID 129,712,718 | CID 129,712,856 | CID 129,713,733 | CID 129,713,737 |
| CID 129,713,738 | CID 129,713,739 | CID 129,717,266 | CID 129,718,758 | CID 129,719,237 | CID 129,719,562 |
| CID 129,719,800 | CID 129,719,820 | CID 129,720,038 | CID 129,722,574 | CID 129,722,775 | CID 129,722,776 |
| CID 129,723,383 | CID 129,723,491 | CID 129,723,494 | CID 129,723,497 | CID 129,729,158 | CID 129,731,801 |
| CID 129,733,738 | CID 129,735,433 | CID 129,735,730 | CID 129,736,660 | CID 129,738,079 | CID 129,761,662 |
| CID 129,773,084 | CID 129,774,159 | CID 129,777,886 | CID 129,778,385 | CID 129,779,295 | CID 129,779,321 |
| CID 129,798,633 | CID 129,803,712 | CID 129,804,869 | CID 129,805,741 | CID 129,811,014 | CID 129,812,676 |
| CID 129,812,778 | CID 129,812,969 | CID 129,813,068 | CID 129,821,519 | CID 129,827,070 | CID 129,830,120 |
| CID 129,831,773 | CID 129,831,774 | CID 129,834,223 | CID 129,837,610 | CID 129,837,611 | CID 129,841,661 |
| CID 129,841,839 | CID 129,841,933 | CID 129,844,962 | CID 129,846,552 | CID 129,847,296 | CID 129,847,546 |
| CID 129,849,055 | CID 129,849,662 | CID 129,850,135 | CID 123,850,275 | CID 129,857,182 | CID 129,864,519 |
| CID 129,885,100 | CID 129,888,841 | CID 129,889,451 | CID 129,891,425 | CID 129,893,859 | CID 130,476,815 |
| CID 131,664,348 | CID 131,667,318 | CID 131,698,710 | CID 131,704,736 | CID 131,706,375 | CID 131,707,539 |
| CID 131,726,110 | CID 131,726,111 | CID 131,734,265 | CID 131,842,801 | CID 131,853,020 | CID 131,853,184 |
| CID 131,854,974 | CID 131,855,532 | CID 131,856,051 | CID 131,856,316 | CID 131,858,650 | CID 131,859,300 |
| CID 131,861,196 | CID 131,861,243 | CID 131,861,284 | CID 131,861,319 | CID 131,861,545 | CID 131,861,741 |
| CID 131,861,783 | CID 131,861,797 | CID 131,861,798 | CID 131,861,829 | CID 131,861,848 | CID 131,861,849 |
| CID 131,861,850 | CID 131,861,864 | CID 131,861,914 | CID 131,862,031 | CID 131,862,032 | CID 131,862,422 |
| CID 131,862,475 | CID 131,862,482 | CID 131,862,601 | CID 131,862,602 | CID 131,864,343 | CID 131,864,629 |
| CID 131,864,631 | CID 131,872,529 | CID 131,872,532 | CID 131,872,533 | CID 131,872,534 | CID 131,872,599 |
| CID 131,872,605 | CID 131,872,607 | CID 131,872,608 | CID 131,872,641 | CID 131,873,917 | CID 131,874,100 |
| CID 131,874,290 | CID 131,874,697 | CID 131,876,571 | CID 131,876,623 | CID 131,876,656 | CID 131,877,220 |
| CID 131,877,514 | CID 131,877,809 | CID 131,878,335 | CID 131,878,542 | CID 131,878,865 | CID 131,879,236 |
| CID 131,880,496 | CID 131,880,551 | CID 131,880,593 | CID 131,880,687 | CID 131,880,733 | CID 131,880,759 |
| CID 131,880,794 | CID 131,880,804 | CID 131,881,065 | CID 131,881,441 | CID 131,881,809 | CID 131,881,834 |
| CID 131,881,910 | CID 131,882,628 | CID 131,882,923 | CID 131,884,410 | CID 131,885,054 | CID 131,885,076 |
| CID 131,885,077 | CID 131,885,482 | CID 131,887,556 | CID 131,887,990 | CID 131,888,557 | |

TABLE 3
558 three valence bismuth (“Bi”) containing compounds were predicted
to efficiently bind PANDA Pocket and efficiently rescue structural mp53.
All of the 94.2 million structures recorded in PubChem
(https://pubchem.ncbi.nlm.nih.gov/) were applied for 4C+ screening.
In the 4C+ screening, we collected those with more than 2 cysteine-binding
potential. Carbon-binding As/Sb/Bi bond has defect in binding cysteine
since this bond cannot be hydrolyzed. The other As/Sb/Bi bond can be
hydrolyzed in cells and thus is able to bind cysteine.
Bi 3 Valence PANDA Agents

| | | | | | |
|---|---|---|---|---|---|
| CID 6,879 | CID 9,010 | CID 9,242 | CID 14,776 | CID 24,591 | CID 29,573 |
| CID 82,232 | CID 82,233 | CID 111,041 | CID 111,042 | CID 273,108 | CID 409,574 |
| CID 438,310 | CID 554,966 | CID 560,564 | CID 3,264,969 | CID 3,310,373 | CID 3,353,258 |
| CID 3,693,500 | CID 3,826,913 | CID 3,835,619 | CID 5,219,607 | CID 5,351,543 | CID 6,097,053 |
| CID 6,101,599 | CID 6,327,012 | CID 6,327,096 | CID 6,327,902 | CID 6,328,044 | CID 6,328,058 |
| CID 6,328,061 | CID 6,328,064 | CID 6,328,110 | CID 6,328,152 | CID 6,328,166 | CID 6,328,191 |
| CID 6,328,192 | CID 6,328,644 | CID 6,328,667 | CID 6,328,676 | CID 6,328,741 | CID 6,329,119 |
| CID 6,329,123 | CID 6,330,910 | CID 6,330,912 | CID 6,331,875 | CID 6,332,255 | CID 6,334,573 |
| CID 6,335,194 | CID 6,335,198 | CID 6,335,206 | CID 6,335,254 | CID 6,335,359 | CID 6,335,607 |
| CID 6,336,257 | CID 6,338,042 | CID 6,365,054 | CID 6,365,215 | CID 6,365,241 | CID 6,367,060 |
| CID 6,367,061 | CID 6,367,062 | CID 6,367,063 | CID 6,367,235 | CID 6,368,739 | CID 6,369,167 |
| CID 6,374,651 | CID 6,378,840 | CID 6,379,155 | CID 6,379,269 | CID 6,379,609 | CID 6,381,732 |
| CID 6,386,980 | CID 6,391,530 | CID 6,397,242 | CID 6,711,587 | CID 6,832,148 | CID 6,914,522 |
| CID 6,914,523 | CID 6,914,524 | CID 6,914,525 | CID 9,804,891 | CID 9,810,547 | CID 9,942,004 |
| CID 9,987,441 | CID 10,053,092 | CID 10,054,529 | CID 10,246,425 | CID 10,327,332 | CID 10,391,773 |
| CID 10,717,929 | CID 10,832,460 | CID 11,049,781 | CID 11,069,084 | CID 11,073,147 | CID 11,146,955 |
| CID 11,284,230 | CID 11,366,706 | CID 11,411,567 | CID 11,528,063 | CID 11,643,620 | CID 11,686,583 |
| CID 11,765,348 | CID 11,786,056 | CID 11,979,775 | CID 12,147,504 | CID 12,622,640 | CID 13,751,291 |
| CID 13,766,272 | CID 13,775,335 | CID 13,775,336 | CID 13,828,286 | CID 13,908,690 | CID 14,044,341 |
| CID 14,044,344 | CID 14,085,833 | CID 14,619,600 | CID 14,849,493 | CID 15,240,258 | CID 15,240,263 |
| CID 15,328,170 | CID 15,770,557 | CID 15,770,559 | CID 15,817,730 | CID 16,132,801 | CID 16,132,845 |
| CID 16,132,857 | CID 16,132,866 | CID 16,132,889 | CID 16,133,169 | CID 16,212,591 | CID 16,212,592 |
| CID 16,682,734 | CID 16,682,825 | CID 16,682,928 | CID 16,682,937 | CID 16,682,955 | CID 16,682,959 |
| CID 16,682,960 | CID 16,682,976 | CID 16,682,977 | CID 16,682,999 | CID 16,683,015 | CID 16,683,017 |
| CID 16,683,095 | CID 16,683,098 | CID 16,683,103 | CID 16,683,121 | CID 16,683,563 | CID 16,683,564 |
| CID 16,683,565 | CID 16,683,566 | CID 16,683,596 | CID 16,683,686 | CID 16,683,874 | CID 16,683,875 |
| CID 16,683,958 | CID 16,683,961 | CID 16,683,963 | CID 16,684,025 | CID 16,684,114 | CID 16,684,115 |
| CID 16,684,274 | CID 16,684,350 | CID 16,684,468 | CID 16,684,575 | CID 16,684,578 | CID 16,684,580 |
| CID 16,684,582 | CID 16,684,785 | CID 16,684,795 | CID 16,684,898 | CID 16,685,021 | CID 16,685,033 |
| CID 16,685,173 | CID 16,685,257 | CID 16,685,276 | CID 16,685,277 | CID 16,685,391 | CID 16,685,632 |
| CID 16,685,998 | CID 16,686,097 | CID 16,686,099 | CID 16,686,175 | CID 16,686,256 | CID 16,686,258 |
| CID 16,686,506 | CID 16,687,246 | CID 16,687,501 | CID 16,688,082 | CID 16,688,103 | CID 16,688,295 |
| CID 16,688,699 | CID 16,688,996 | CID 16,689,505 | CID 16,689,550 | CID 16,689,947 | CID 16,693,013 |
| CID 16,693,015 | CID 16,695,048 | CID 16,695,049 | CID 16,696,198 | CID 16,697,869 | CID 16,697,870 |
| CID 16,697,871 | CID 16,697,873 | CID 16,699,463 | CID 16,700,590 | CID 16,700,595 | CID 16,700,901 |
| CID 16,701,354 | CID 16,701,355 | CID 16,702,113 | CID 16,702,492 | CID 16,702,987 | CID 16,703,065 |
| CID 16,704,191 | CID 16,704,973 | CID 16,704,976 | CID 16,704,977 | CID 16,704,984 | CID 16,704,997 |
| CID 16,705,083 | CID 16,705,860 | CID 16,707,734 | CID 16,711,827 | CID 16,712,566 | CID 16,716,635 |
| CID 16,717,608 | CID 16,717,608 | CID 16,717,622 | CID 16,721,198 | CID 18,502,954 | CID 18,503,058 |
| CID 18,503,101 | CID 18,503,123 | CID 18,503,172 | CID 18,503,263 | CID 18,503,264 | CID 18,503,288 |
| CID 18,503,517 | CID 18,503,857 | CID 18,690,641 | CID 18,690,642 | CID 21,932,960 | CID 21,932,966 |
| CID 21,932,967 | CID 21,932,971 | CID 21,932,984 | CID 21,932,998 | CID 21,933,008 | CID 21,933,012 |
| CID 21,933,025 | CID 21,933,028 | CID 21,933,044 | CID 21,933,048 | CID 21,933,092 | CID 21,933,118 |
| CID 21,933,120 | CID 21,933,124 | CID 21,933,126 | CID 21,933,129 | CID 21,933,167 | CID 21,933,168 |
| CID 21,933,169 | CID 21,933,179 | CID 21,933,244 | CID 21,933,252 | CID 21,933,277 | CID 21,933,284 |
| CID 21,933,317 | CID 21,933,328 | CID 21,933,329 | CID 21,933,331 | CID 21,933,335 | CID 21,933,358 |
| CID 21,933,362 | CID 21,933,365 | CID 21,933,367 | CID 21,933,368 | CID 22,834,097 | CID 22,834,234 |
| CID 22,834,266 | CID 22,834,406 | CID 22,834,407 | CID 22,929,628 | CID 23,230,204 | CID 23,576,945 |
| CID 24,182,960 | CID 24,184,948 | CID 24,771,807 | CID 24,884,204 | CID 24,884,205 | CID 25,021,316 |
| CID 42,619,967 | CID 44,135,860 | CID 44,135,899 | CID 44,146,529 | CID 44,152,487 | CID 45,052,077 |
| CID 46,245,067 | CID 50,912,221 | CID 50,920,714 | CID 50,920,757 | CID 50,931,875 | CID 53,393,588 |
| CID 53,427,529 | CID 54,605,444 | CID 54,742,562 | CID 56,841,599 | CID 56,842,096 | CID 56,842,896 |
| CID 56,845,529 | CID 56,845,924 | CID 56,846,073 | CID 56,846,074 | CID 56,846,075 | CID 56,846,076 |
| CID 57,347,031 | CID 57,357,928 | CID 57,404,116 | CID 57,488,589 | CID 57,562,349 | CID 58,330,672 |
| CID 59,499,197 | CID 59,720,413 | CID 59,720,414 | CID 70,294,115 | CID 71,300,497 | CID 71,309,993 |
| CID 71,310,157 | CID 71,310,700 | CID 71,311,500 | CID 71,380,213 | CID 71,380,459 | CID 71,386,040 |
| CID 71,391,524 | CID 71,400,325 | CID 73,557,522 | CID 73,894,347 | CID 73,894,349 | CID 73,894,350 |
| CID 73,995,040 | CID 73,995,041 | CID 76,959,357 | CID 85,470,850 | CID 85,470,850 | CID 85,470,851 |
| CID 85,624,350 | CID 85,750,126 | CID 87,126,125 | CID 87,207,683 | CID 87,207,773 | CID 87,207,774 |
| CID 87,238,523 | CID 87,452,858 | CID 87,479,654 | CID 87,489,025 | CID 87,489,305 | CID 87,489,320 |
| CID 87,489,335 | CID 87,489,593 | CID 87,489,619 | CID 87,489,650 | CID 87,489,777 | CID 87,490,196 |
| CID 87,490,221 | CID 87,490,477 | CID 87,490,638 | CID 87,490,646 | CID 87,490,651 | CID 87,490,697 |
| CID 87,490,956 | CID 87,490,958 | CID 87,491,355 | CID 87,491,385 | CID 87,491,423 | CID 87,513,516 |
| CID 87,513,517 | CID 87,580,507 | CID 87,686,514 | CID 87,686,718 | CID 87,687,197 | CID 87,687,712 |
| CID 87,693,276 | CID 87,730,138 | CID 87,730,829 | CID 87,737,505 | CID 87,745,214 | CID 87,871,946 |
| CID 87,871,947 | CID 87,912,467 | CID 87,912,468 | CID 87,960,355 | CID 88,029,006 | CID 88,029,007 |
| CID 88,193,567 | CID 88,194,406 | CID 88,194,416 | CID 88,194,477 | CID 88,194,479 | CID 88,194,481 |
| CID 88,194,539 | CID 88,194,861 | CID 88,194,864 | CID 88,194,865 | CID 88,194,870 | CID 88,292,939 |
| CID 88,292,940 | CID 88,520,030 | CID 88,640,276 | CID 88,640,277 | CID 88,802,217 | CID 88,802,218 |
| CID 88,817,848 | CID 88,836,364 | CID 90,471,463 | CID 90,471,464 | CID 90,473,777 | CID 90,473,778 |
| CID 90,475,361 | CID 91,659,154 | CID 91,886,165 | CID 91,886,166 | CID 91,886,248 | CID 91,886,395 |
| CID 91,886,591 | CID 92,003,295 | CID 92,024,600 | CID 92,025,641 | CID 92,025,642 | CID 92,025,643 |
| CID 92,025,667 | CID 92,025,668 | CID 92,025,669 | CID 92,025,670 | CID 92,025,671 | CID 92,025,673 |
| CID 92,025,675 | CID 92,025,675 | CID 92,026,749 | CID 92,026,962 | CID 102,602,549 | CID 102,602,549 |
| CID 117,065,228 | CID 117,071,502 | CID 118,855,609 | CID 118,855,610 | CID 118,985,207 | CID 121,233,070 |
| CID 121,233,129 | CID 121,233,209 | CID 121,233,210 | CID 121,233,689 | CID 121,233,709 | CID 121,233,710 |
| CID 121,513,973 | CID 122,173,805 | CID 122,173,806 | CID 129,627,851 | CID 129,627,851 | CID 129,627,852 |
| CID 129,627,852 | CID 129,628,345 | CID 129,628,507 | CID 129,630,444 | CID 129,631,009 | CID 129,631,020 |
| CID 129,631,047 | CID 129,632,182 | CID 129,632,246 | CID 129,632,626 | CID 129,632,968 | CID 129,634,022 |
| CID 129,636,151 | CID 129,636,165 | CID 129,636,935 | CID 129,636,953 | CID 129,637,329 | CID 129,643,765 |
| CID 129,643,766 | CID 129,644,127 | CID 129,651,463 | CID 129,651,702 | CID 129,651,972 | CID 129,660,613 |
| CID 129,661,690 | CID 129,661,691 | CID 129,661,692 | CID 129,662,722 | CID 129,665,855 | CID 129,670,802 |
| CID 129,671,760 | CID 129,672,684 | CID 129,676,730 | CID 129,692,302 | CID 129,693,004 | CID 129,693,466 |
| CID 129,696,449 | CID 129,713,086 | CID 129,713,109 | CID 129,713,799 | CID 129,719,991 | CID 129,722,002 |
| CID 129,734,931 | CID 129,738,069 | CID 129,738,070 | CID 129,738,237 | CID 129,759,637 | CID 129,759,638 |
| CID 129,760,053 | CID 129,760,197 | CID 129,760,222 | CID 129,760,768 | CID 129,761,066 | CID 129,761,789 |
| CID 129,761,805 | CID 129,761,822 | CID 129,768,269 | CID 129,769,201 | CID 129,772,739 | CID 129,772,785 |
| CID 129,772,858 | CID 129,773,065 | CID 129,793,750 | CID 129,796,408 | CID 129,796,934 | CID 129,802,846 |
| CID 129,802,847 | CID 129,817,421 | CID 129,819,068 | CID 129,822,257 | CID 129,822,258 | CID 129,831,438 |
| CID 129,842,132 | CID 129,842,163 | CID 129,842,182 | CID 129,842,230 | CID 129,842,243 | CID 129,842,265 |
| CID 129,842,280 | CID 129,843,462 | CID 129,843,548 | CID 129,843,683 | CID 129,851,323 | CID 129,851,730 |
| CID 129,852,640 | CID 129,856,536 | CID 129,856,537 | CID 129,865,810 | CID 129,880,632 | CID 129,880,679 |
| CID 129,887,215 | CID 129,890,689 | CID 129,891,194 | CID 129,891,993 | CID 131,864,282 | CID 131,875,075 |

TABLE 4
125 five valence antimony (“Sb”) structures were predicted
to efficiently bind PANDA Pocket and efficiently rescue structural mp53.
All of the 94.2 million structures recorded in PubChem
(https://pubchem.ncbi.nlm.nih.gov/) were applied for 4C+ screening.
In the 4C+ screening, we collected those with more than 2 cysteine-binding
potential. Carbon-binding As/Sb/Bi bond has defect in binding cysteine
since this bond cannot be hydrolyzed. The other As/Sb/Bi bond can be
hydrolyzed in cells and thus is able to bind cysteine.
Bi 5 Valence PANDA Agents

| | | | | | |
|---|---|---|---|---|---|
| CID 82,993 | CID 123,260 | CID 366,096 | CID 366,097 | CID 366,098 | CID 366,099 |
| CID 367,796 | CID 434,309 | CID 438,389 | CID 2,734,035 | CID 3,301,555 | CID 3,336,805 |
| CID 3,538,901 | CID 3,653,008 | CID 3,687,219 | CID 3,783,338 | CID 3,914,869 | CID 3,992,551 |
| CID 4,063,671 | CID 4,271,061 | CID 4,441,160 | CID 5,254,690 | CID 5,256,620 | CID 5,257,028 |
| CID 5,257,050 | CID 5,257,056 | CID 5,257,058 | CID 5,257,059 | CID 5,257,062 | CID 5,257,063 |
| CID 6,327,785 | CID 6,334,091 | CID 6,372,954 | CID 6,373,400 | CID 6,377,431 | CID 6,386,952 |
| CID 6,392,480 | CID 6,392,684 | CID 6,392,717 | CID 6,392,777 | CID 6,394,267 | CID 6,394,856 |
| CID 6,394,889 | CID 6,395,066 | CID 6,395,342 | CID 6,395,344 | CID 6,395,345 | CID 6,395,477 |
| CID 6,396,057 | CID 6,396,714 | CID 6,397,027 | CID 6,397,420 | CID 6,397,557 | CID 6,711,667 |
| CID 6,711,688 | CID 6,711,693 | CID 6,850,086 | CID 6,850,087 | CID 6,850,113 | CID 10,907,992 |
| CID 11,188,826 | CID 11,966,239 | CID 11,968,204 | CID 11,980,596 | CID 11,980,909 | CID 11,985,775 |
| CID 11,985,985 | CID 14,420,964 | CID 16,132,606 | CID 16,132,606 | CID 16,132,662 | CID 16,132,822 |
| CID 16,132,858 | CID 16,132,869 | CID 16,132,960 | CID 16,132,965 | CID 16,133,022 | CID 16,133,325 |
| CID 16,133,326 | CID 16,682,751 | CID 16,684,917 | CID 16,685,090 | CID 16,685,106 | CID 16,685,289 |
| CID 16,685,303 | CID 16,685,390 | CID 16,685,453 | CID 16,685,454 | CID 16,685,455 | CID 16,685,456 |
| CID 16,685,573 | CID 16,685,574 | CID 16,685,631 | CID 16,685,637 | CID 16,715,250 | CID 18,503,128 |
| CID 18,503,206 | CID 19,032,053 | CID 21,554,417 | CID 21,554,418 | CID 21,944,623 | CID 23,304,890 |
| CID 23,304,944 | CID 23,350,967 | CID 23,350,968 | CID 23,633,148 | CID 23,639,873 | CID 23,681,527 |
| CID 23,693,098 | CID 50,920,763 | CID 53,249,975 | CID 58,280,986 | CID 71,357,908 | CID 71,401,130 |
| CID 87,744,887 | CID 87,745,037 | CID 87,745,510 | CID 88,228,697 | CID 88,515,761 | CID 88,797,100 |
| CID 91,886,318 | CID 124,202,748 | CID 129,830,992 | CID 131,864,281 | CID 131,868,916 | |

TABLE 5
937 three valence bismuth (“Bi”) structures were predicted
to efficiently bind PANDA Pocket and efficiently rescue structural mp53.
All of the 94.2 million structures recorded in PubChem
(https://pubchem.ncbi.nlm.nih.gov/) were applied for 4C+ screening.
In the 4C+ screening, we collected those with more than 2 cysteine-binding
potential. Carbon-binding As/Sb/Bi bond has defect in binding cysteine
since this bond cannot be hydrolyzed. The other As/Sb/Bi bond can be
hydrolyzed in cells and thus is able to bind cysteine.
Sb 3 Valence PANDA Agents

| | | | | | |
|---|---|---|---|---|---|
| CID 9,359 | CID 18,193 | CID 24,554 | CID 24,615 | CID 24,630 | CID 24,814 |
| CID 27,652 | CID 46,678 | CID 61,553 | CID 72,062 | CID 82,258 | CID 83,753 |
| CID 85,159 | CID 85,168 | CID 91,307 | CID 92,908 | CID 110,797 | CID 110,822 |
| CID 112,343 | CID 112,417 | CID 116,971 | CID 117,333 | CID 117,654 | CID 120,130 |
| CID 122,445 | CID 139,227 | CID 172,734 | CID 201,832 | CID 223,838 | CID 231,056 |
| CID 273,193 | CID 278,175 | CID 292,777 | CID 432,482 | CID 443,987 | CID 2,724,507 |
| CID 3,022,556 | CID 3,468,413 | CID 3,500,394 | CID 3,980,757 | CID 4,003,909 | CID 4,169,194 |
| CID 4,227,894 | CID 4,310,207 | CID 4,426,282 | CID 4,868,265 | CID 5,099,079 | CID 5,205,981 |
| CID 5,460,498 | CID 5,460,499 | CID 5,475,465 | CID 6,100,615 | CID 6,100,855 | CID 6,102,344 |
| CID 6,327,063 | CID 6,327,128 | CID 6,327,518 | CID 6,327,644 | CID 6,327,672 | CID 6,327,776 |
| CID 6,327,782 | CID 6,327,783 | CID 6,327,784 | CID 6,327,790 | CID 6,327,791 | CID 6,327,792 |
| CID 6,327,901 | CID 6,328,047 | CID 6,328,136 | CID 6,328,158 | CID 6,328,167 | CID 6,328,183 |
| CID 6,328,247 | CID 6,328,381 | CID 6,328,497 | CID 6,328,498 | CID 6,328,604 | CID 6,328,723 |
| CID 6,328,778 | CID 6,329,083 | CID 6,329,280 | CID 6,329,469 | CID 6,330,102 | CID 6,331,892 |
| CID 6,331,893 | CID 6,331,894 | CID 6,332,059 | CID 6,333,990 | CID 6,334,205 | CID 6,334,206 |
| CID 6,335,210 | CID 6,335,446 | CID 6,335,642 | CID 6,335,799 | CID 6,335,965 | CID 6,335,967 |
| CID 6,335,971 | CID 6,336,248 | CID 6,336,250 | CID 6,336,268 | CID 6,336,294 | CID 6,336,294 |
| CID 6,336,295 | CID 6,336,297 | CID 6,336,298 | CID 6,336,309 | CID 6,336,310 | CID 6,336,314 |
| CID 6,336,315 | CID 6,336,966 | CID 6,337,127 | CID 6,337,192 | CID 6,338,235 | CID 6,338,236 |
| CID 6,338,237 | CID 6,338,238 | CID 6,338,239 | CID 6,338,275 | CID 6,338,396 | CID 6,338,574 |
| CID 6,366,253 | CID 6,367,067 | CID 6,367,121 | CID 6,367,122 | CID 6,367,123 | CID 6,367,297 |
| CID 6,367,299 | CID 6,369,653 | CID 6,371,267 | CID 6,372,250 | CID 6,372,531 | CID 6,373,293 |
| CID 6,378,487 | CID 6,378,591 | CID 6,379,063 | CID 6,380,952 | CID 6,383,190 | CID 6,383,685 |
| CID 6,383,702 | CID 6,386,140 | CID 6,389,340 | CID 6,391,662 | CID 6,391,766 | CID 6,392,060 |
| CID 6,393,025 | CID 6,394,845 | CID 6,394,846 | CID 6,394,847 | CID 6,394,849 | CID 6,395,364 |
| CID 6,395,540 | CID 6,395,541 | CID 6,395,542 | CID 6,395,614 | CID 6,395,616 | CID 6,397,381 |
| CID 6,398,008 | CID 6,398,524 | CID 6,398,617 | CID 6,398,618 | CID 6,416,685 | CID 6,437,746 |
| CID 6,450,406 | CID 6,450,407 | CID 6,451,277 | CID 6,452,001 | CID 6,857,635 | CID 6,914,521 |
| CID 6,914,522 | CID 6,914,523 | CID 6,914,524 | CID 6,914,526 | CID 6,914,527 | CID 9,986,376 |
| CID 9,988,372 | CID 10,078,981 | CID 10,440,350 | CID 10,508,768 | CID 10,604,595 | CID 10,652,047 |
| CID 10,723,372 | CID 10,818,460 | CID 10,876,667 | CID 10,887,720 | CID 10,939,671 | CID 10,953,054 |
| CID 11,005,471 | CID 11,018,607 | CID 11,028,580 | CID 11,038,092 | CID 11,039,089 | CID 11,088,054 |
| CID 11,091,142 | CID 11,181,852 | CID 11,193,223 | CID 11,215,665 | CID 11,216,896 | CID 11,400,464 |
| CID 11,416,121 | CID 11,479,469 | CID 11,489,983 | CID 11,542,349 | CID 11,600,329 | CID 11,643,503 |
| CID 11,801,623 | CID 11,824,030 | CID 11,954,289 | CID 11,968,245 | CID 11,969,034 | CID 11,979,399 |
| CID 12,545,033 | CID 13,100,649 | CID 13,165,617 | CID 13,706,358 | CID 14,085,828 | CID 14,085,832 |
| CID 14,766,829 | CID 14,923,299 | CID 15,165,103 | CID 15,246,215 | CID 15,274,122 | CID 15,630,343 |
| CID 15,773,247 | CID 15,779,436 | CID 15,815,188 | CID 16,132,617 | CID 16,132,626 | CID 16,132,842 |
| CID 16,132,972 | CID 16,682,736 | CID 16,682,737 | CID 16,682,742 | CID 16,682,744 | CID 16,682,747 |
| CID 16,682,749 | CID 16,682,752 | CID 16,682,753 | CID 16,682,754 | CID 16,682,822 | CID 16,682,940 |
| CID 16,682,941 | CID 16,682,985 | CID 16,682,986 | CID 16,682,996 | CID 16,683,002 | CID 16,683,008 |
| CID 16,683,009 | CID 16,683,047 | CID 16,683,067 | CID 16,683,068 | CID 16,683,082 | CID 16,683,091 |
| CID 16,683,110 | CID 16,683,184 | CID 16,683,603 | CID 16,683,605 | CID 16,683,607 | CID 16,683,654 |
| CID 16,683,656 | CID 16,683,857 | CID 16,683,859 | CID 16,683,966 | CID 16,683,967 | CID 16,684,084 |
| CID 16,684,126 | CID 16,684,127 | CID 16,684,162 | CID 16,684,169 | CID 16,684,206 | CID 16,684,210 |
| CID 16,684,264 | CID 16,684,266 | CID 16,684,267 | CID 16,684,268 | CID 16,684,270 | CID 16,684,295 |
| CID 16,684,303 | CID 16,684,309 | CID 16,684,377 | CID 16,684,378 | CID 16,684,379 | CID 16,684,380 |
| CID 16,684,381 | CID 16,684,383 | CID 16,684,474 | CID 16,684,490 | CID 16,684,491 | CID 16,684,542 |
| CID 16,684,587 | CID 16,684,616 | CID 16,684,617 | CID 16,684,618 | CID 16,684,619 | CID 16,684,620 |
| CID 16,684,622 | CID 16,684,713 | CID 16,684,714 | CID 16,684,728 | CID 16,684,732 | CID 16,684,860 |
| CID 16,684,861 | CID 16,684,878 | CID 16,684,889 | CID 16,685,013 | CID 16,685,014 | CID 16,685,015 |
| CID 16,685,080 | CID 16,685,138 | CID 16,685,151 | CID 16,685,153 | CID 16,685,185 | CID 16,685,197 |
| CID 16,685,221 | CID 16,685,265 | CID 16,685,272 | CID 16,685,273 | CID 16,685,311 | CID 16,685,415 |
| CID 16,685,416 | CID 16,685,417 | CID 16,685,418 | CID 16,685,479 | CID 16,685,481 | CID 16,685,497 |
| CID 16,685,764 | CID 16,686,007 | CID 16,686,173 | CID 16,686,176 | CID 16,686,177 | CID 16,686,294 |
| CID 16,686,575 | CID 16,686,576 | CID 16,687,650 | CID 16,687,895 | CID 16,688,146 | CID 16,688,454 |
| CID 16,688,473 | CID 16,688,527 | CID 16,688,544 | CID 16,688,545 | CID 16,688,698 | CID 16,688,732 |
| CID 16,688,935 | CID 16,688,973 | CID 16,688,974 | CID 16,688,975 | CID 16,689,006 | CID 16,689,163 |
| CID 16,689,752 | CID 16,691,582 | CID 16,693,615 | CID 16,693,637 | CID 16,693,639 | CID 16,693,641 |
| CID 16,694,223 | CID 16,695,174 | CID 16,695,175 | CID 16,695,950 | CID 16,695,951 | CID 16,695,952 |
| CID 16,697,056 | CID 16,697,515 | CID 16,700,667 | CID 16,700,668 | CID 16,700,670 | CID 16,700,811 |
| CID 16,700,812 | CID 16,700,896 | CID 16,701,020 | CID 16,703,714 | CID 16,705,235 | CID 16,706,597 |
| CID 16,708,103 | CID 16,708,935 | CID 16,709,013 | CID 16,709,919 | CID 16,711,745 | CID 16,712,315 |
| CID 16,713,256 | CID 16,716,705 | CID 16,738,697 | CID 16,760,657 | CID 17,749,634 | CID 17,757,230 |
| CID 17,757,257 | CID 17,896,854 | CID 17,907,305 | CID 18,347,240 | CID 18,502,908 | CID 18,502,961 |
| CID 18,502,962 | CID 18,503,115 | CID 18,503,122 | CID 18,503,252 | CID 18,503,253 | CID 18,503,254 |
| CID 18,503,313 | CID 18,503,565 | CID 18,503,729 | CID 18,503,753 | CID 18,690,650 | CID 18,690,654 |
| CID 18,690,656 | CID 18,690,658 | CID 19,097,033 | CID 19,097,037 | CID 19,821,447 | CID 19,933,062 |
| CID 20,111,700 | CID 20,185,678 | CID 20,249,102 | CID 20,249,105 | CID 20,249,108 | CID 20,249,113 |
| CID 20,249,115 | CID 20,300,474 | CID 20,313,258 | CID 20,315,019 | CID 20,363,281 | CID 20,435,550 |
| CID 20,650,165 | CID 20,650,168 | CID 20,650,180 | CID 20,836,036 | CID 20,838,936 | CID 20,840,786 |
| CID 20,840,787 | CID 20,841,413 | CID 20,841,414 | CID 21,127,957 | CID 21,127,960 | CID 21,162,914 |
| CID 21,162,915 | CID 21,162,916 | CID 21,179,954 | CID 21,280,137 | CID 21,387,935 | CID 21,429,702 |
| CID 21,431,092 | CID 21,597,739 | CID 21,597,740 | CID 21,597,742 | CID 21,597,743 | CID 21,597,747 |
| CID 21,853,875 | CID 21,853,876 | CID 21,853,877 | CID 21,853,878 | CID 21,853,880 | CID 21,853,882 |
| CID 21,853,884 | CID 21,924,199 | CID 21,946,953 | CID 21,976,042 | CID 22,134,008 | CID 22,476,815 |
| CID 22,483,778 | CID 22,755,405 | CID 22,755,406 | CID 22,755,407 | CID 22,755,408 | CID 22,755,409 |
| CID 22,755,410 | CID 22,834,430 | CID 23,089,770 | CID 23,232,432 | CID 23,262,264 | CID 23,262,275 |
| CID 23,262,289 | CID 23,262,291 | CID 23,262,328 | CID 23,262,329 | CID 23,271,407 | CID 23,271,424 |
| CID 23,271,440 | CID 23,271,479 | CID 23,412,698 | CID 23,412,699 | CID 23,412,700 | CID 23,412,701 |
| CID 23,412,702 | CID 23,412,703 | CID 23,412,704 | CID 23,412,705 | CID 23,412,706 | CID 23,412,707 |
| CID 23,412,708 | CID 23,412,709 | CID 23,412,710 | CID 23,412,711 | CID 23,412,744 | CID 23,412,745 |
| CID 23,412,746 | CID 23,412,747 | CID 23,424,127 | CID 23,452,096 | CID 23,617,918 | CID 23,665,405 |
| CID 23,667,272 | CID 23,675,780 | CID 23,681,183 | CID 23,686,990 | CID 23,690,288 | CID 23,707,960 |
| CID 23,714,520 | CID 24,182,330 | CID 24,182,331 | CID 24,199,786 | CID 24,201,065 | CID 24,837,728 |
| CID 24,847,360 | CID 24,867,558 | CID 24,884,257 | CID 25,021,693 | CID 25,200,065 | CID 44,119,133 |
| CID 44,135,767 | CID 44,135,895 | CID 44,145,400 | CID 44,145,839 | CID 44,150,046 | CID 44,153,415 |
| CID 45,479,364 | CID 45,479,524 | CID 45,479,539 | CID 50,896,902 | CID 50,909,120 | CID 50,918,374 |
| CID 50,921,100 | CID 50,930,621 | CID 50,933,843 | CID 50,935,021 | CID 53,315,432 | CID 53,471,862 |
| CID 53,494,194 | CID 54,603,506 | CID 54,604,975 | CID 54,605,443 | CID 54,611,195 | CID 54,688,499 |
| CID 54,703,985 | CID 54,703,986 | CID 54,703,987 | CID 54,724,826 | CID 54,742,027 | CID 54,750,834 |
| CID 56,845,640 | CID 56,927,675 | CID 57,347,421 | CID 57,348,872 | CID 57,350,497 | CID 57,352,871 |
| CID 57,357,960 | CID 57,370,241 | CID 57,371,215 | CID 57,490,232 | CID 57,645,580 | CID 57,704,204 |
| CID 57,704,207 | CID 57,731,111 | CID 57,731,115 | CID 57,731,116 | CID 57,731,118 | CID 57,731,120 |
| CID 57,731,121 | CID 57,731,122 | CID 57,789,471 | CID 57,789,472 | CID 57,953,647 | CID 58,253,024 |
| CID 58,253,026 | CID 58,253,027 | CID 58,253,029 | CID 58,253,030 | CID 58,253,031 | CID 58,253,032 |
| CID 58,271,553 | CID 58,271,555 | CID 58,271,564 | CID 58,271,575 | CID 58,271,579 | CID 58,271,583 |
| CID 58,280,987 | CID 58,280,988 | CID 58,280,990 | CID 58,288,679 | CID 58,609,137 | CID 58,720,422 |
| CID 59,032,477 | CID 59,086,320 | CID 59,159,883 | CID 59,159,883 | CID 59,499,187 | CID 59,499,195 |
| CID 59,499,196 | CID 59,499,199 | CID 59,499,204 | CID 59,557,632 | CID 59,571,971 | CID 59,571,972 |
| CID 59,891,573 | CID 59,891,601 | CID 59,891,608 | CID 59,891,640 | CID 71,301,022 | CID 71,301,023 |
| CID 71,301,024 | CID 71,333,883 | CID 71,342,634 | CID 71,345,945 | CID 71,345,945 | CID 71,345,946 |
| CID 71,359,975 | CID 71,361,093 | CID 71,363,456 | CID 71,367,105 | CID 71,374,340 | CID 71,380,591 |
| CID 71,429,674 | CID 71,441,094 | CID 71,442,382 | CID 72,720,464 | CID 72,720,468 | CID 73,307,702 |
| CID 73,307,769 | CID 73,307,770 | CID 73,307,825 | CID 73,307,873 | CID 73,555,373 | CID 73,555,376 |
| CID 73,555,379 | CID 73,555,501 | CID 73,555,893 | CID 73,557,535 | CID 73,759,938 | CID 73,894,305 |
| CID 73,894,308 | CID 73,894,311 | CID 73,894,312 | CID 73,894,313 | CID 73,894,314 | CID 73,894,315 |
| CID 73,894,323 | CID 73,952,085 | CID 73,995,019 | CID 73,995,020 | CID 74,040,665 | CID 74,765,653 |
| CID 74,933,683 | CID 74,935,384 | CID 76,037,526 | CID 76,960,497 | CID 85,551,321 | CID 85,609,459 |
| CID 85,618,045 | CID 85,750,126 | CID 85,750,126 | CID 85,863,651 | CID 85,863,652 | CID 85,907,651 |
| CID 85,976,002 | CID 86,101,760 | CID 86,101,767 | CID 86,101,784 | CID 86,246,892 | CID 87,138,989 |
| CID 87,186,488 | CID 87,202,814 | CID 87,258,578 | CID 87,261,615 | CID 87,261,620 | CID 87,261,624 |
| CID 87,261,630 | CID 87,261,631 | CID 87,261,715 | CID 87,261,718 | CID 87,261,720 | CID 87,261,724 |
| CID 87,261,726 | CID 87,261,729 | CID 87,261,732 | CID 87,261,737 | CID 87,261,749 | CID 87,261,752 |
| CID 87,315,219 | CID 87,315,760 | CID 87,362,495 | CID 87,378,499 | CID 87,411,324 | CID 87,438,483 |
| CID 87,438,929 | CID 87,450,539 | CID 87,460,730 | CID 87,635,564 | CID 87,702,251 | CID 87,719,536 |
| CID 87,719,538 | CID 87,835,658 | CID 87,857,783 | CID 88,154,076 | CID 88,176,411 | CID 88,176,414 |
| CID 88,261,066 | CID 88,261,223 | CID 88,264,017 | CID 88,264,710 | CID 88,265,017 | CID 88,265,019 |
| CID 88,423,296 | CID 88,430,055 | CID 88,466,104 | CID 88,473,381 | CID 88,484,275 | CID 88,484,276 |
| CID 88,526,235 | CID 88,526,236 | CID 88,526,238 | CID 88,526,252 | CID 88,526,253 | CID 88,613,920 |
| CID 88,613,921 | CID 88,642,592 | CID 88,654,956 | CID 88,654,957 | CID 88,745,663 | CID 88,772,726 |
| CID 88,773,222 | CID 88,773,337 | CID 88,774,019 | CID 88,774,061 | CID 88,777,596 | CID 88,778,787 |
| CID 88,793,603 | CID 88,793,985 | CID 88,795,315 | CID 88,795,398 | CID 88,800,757 | CID 88,801,147 |
| CID 88,801,216 | CID 88,806,748 | CID 88,806,751 | CID 88,820,185 | CID 88,824,737 | CID 90,105,283 |
| CID 90,105,284 | CID 90,471,546 | CID 90,473,139 | CID 90,659,538 | CID 90,659,541 | CID 90,659,637 |
| CID 91,666,614 | CID 91,667,987 | CID 91,668,102 | CID 91,867,127 | CID 91,868,149 | CID 91,886,487 |
| CID 91,980,813 | CID 91,980,814 | CID 91,996,065 | CID 91,997,283 | CID 91,997,284 | CID 91,997,368 |
| CID 91,997,683 | CID 92,004,482 | CID 92,012,267 | CID 92,012,268 | CID 92,023,487 | CID 92,024,179 |
| CID 92,024,528 | CID 92,024,529 | CID 92,024,531 | CID 92,024,534 | CID 92,024,536 | CID 92,024,556 |
| CID 92,024,935 | CID 92,024,944 | CID 92,024,955 | CID 92,024,957 | CID 92,024,958 | CID 92,024,961 |
| CID 92,024,968 | CID 92,024,970 | CID 92,024,971 | CID 92,024,972 | CID 92,024,973 | CID 92,024,976 |
| CID 92,024,977 | CID 92,024,980 | CID 92,024,981 | CID 92,024,982 | CID 92,024,985 | CID 92,024,986 |
| CID 92,024,987 | CID 92,024,988 | CID 92,024,990 | CID 92,024,991 | CID 92,024,997 | CID 92,025,001 |
| CID 92,025,002 | CID 92,025,003 | CID 92,025,020 | CID 92,025,024 | CID 92,025,025 | CID 92,025,026 |
| CID 92,025,027 | CID 92,025,028 | CID 92,025,031 | CID 92,025,050 | CID 92,025,051 | CID 92,025,052 |
| CID 92,025,053 | CID 92,025,054 | CID 92,025,055 | CID 92,025,056 | CID 92,025,057 | CID 92,025,058 |
| CID 92,025,059 | CID 92,025,063 | CID 92,025,064 | CID 92,025,065 | CID 92,025,066 | CID 92,025,067 |
| CID 92,025,069 | CID 92,025,077 | CID 92,025,079 | CID 92,025,082 | CID 92,025,083 | CID 92,025,084 |
| CID 92,025,085 | CID 92,025,086 | CID 92,025,087 | CID 92,025,089 | CID 92,028,426 | CID 92,028,427 |
| CID 92,028,428 | CID 92,028,429 | CID 92,028,430 | CID 92,028,791 | CID 92,028,940 | CID 92,043,602 |
| CID 92,043,603 | CID 102,602,109 | CID 117,065,228 | CID 121,233,627 | CID 124,233,628 | CID 121,596,016 |
| CID 123,132,499 | CID 129,628,368 | CID 129,628,369 | CID 129,628,470 | CID 129,630,836 | CID 129,631,714 |
| CID 129,634,074 | CID 129,634,980 | CID 120,635,876 | CID 129,635,919 | CID 129,636,319 | CID 129,636,320 |
| CID 129,636,621 | CID 129,636,622 | CID 129,636,709 | CID 129,639,940 | CID 129,646,351 | CID 129,649,988 |
| CID 129,664,755 | CID 129,668,929 | CID 129,671,029 | CID 129,671,731 | CID 129,671,732 | CID 129,672,175 |
| CID 129,672,177 | CID 129,672,415 | CID 129,672,416 | CID 129,675,055 | CID 129,676,925 | CID 129,677,475 |
| CID 129,680,159 | CID 129,680,177 | CID 129,680,187 | CID 129,691,639 | CID 129,691,640 | CID 129,697,639 |
| CID 129,703,076 | CID 129,709,779 | CID 129,718,867 | CID 129,719,189 | CID 129,721,729 | CID 129,723,651 |
| CID 129,731,772 | CID 129,731,773 | CID 129,760,451 | CID 129,765,173 | CID 129,765,222 | CID 129,771,692 |
| CID 129,773,098 | CID 129,776,810 | CID 129,781,425 | CID 129,783,529 | CID 129,783,530 | CID 129,794,056 |
| CID 129,798,979 | CID 129,802,873 | CID 129,807,640 | CID 129,809,238 | CID 129,812,117 | CID 129,814,318 |
| CID 129,814,352 | CID 129,814,419 | CID 129,817,665 | CID 129,817,666 | CID 129,821,032 | CID 129,821,963 |
| CID 129,831,304 | CID 129,842,135 | CID 129,842,264 | CID 129,843,508 | CID 129,843,687 | CID 129,848,944 |
| CID 129,852,227 | CID 129,856,127 | CID 129,856,231 | CID 129,864,503 | CID 129,865,814 | CID 129,879,550 |
| CID 129,879,554 | CID 129,886,985 | CID 129,888,228 | CID 129,893,705 | CID 129,893,706 | CID 130,476,776 |
| CID 131,706,152 | CID 131,707,327 | CID 131,852,287 | CID 131,880,394 | CID 131,882,596 | CID 131,882,949 |
| CID 131,885,062 | | | | | |

TABLE 6
1896 five valence bismuth (“Bi”) structures were predicted
to efficiently bind PANDA Pocket and efficiently rescue structural mp53.
All of the 94.2 million structures recorded in PubChem
(https://pubchem.ncbi.nlm.nih.gov/) were applied for 4C+ screening.
In the 4C+ screening, we collected those with more than 2 cysteine-binding
potential. Carbon-binding As/Sb/Bi bond has defect in binding cysteine
since this bond cannot be hydrolyzed. The other As/Sb/Bi bond can be
hydrolyzed in cells and thus is able to bind cysteine.
Sb 5 Valence PANDA Agents

| | | | | | |
|---|---|---|---|---|---|
| CID 11,135 | CID 14,813 | CID 24,294 | CID 24,557 | CID 25,469 | CID 25,470 |
| CID 28,362 | CID 50,592 | CID 61,636 | CID 64,953 | CID 74,002 | CID 93,820 |
| CID 95,060 | CID 116,495 | CID 150,258 | CID 157,275 | CID 182,263 | CID 224,879 |
| CID 224,882 | CID 224,884 | CID 224,885 | CID 224,886 | CID 224,889 | CID 224,895 |
| CID 224,898 | CID 224,899 | CID 224,900 | CID 224,905 | CID 224,909 | CID 224,910 |
| CID 224,913 | CID 224,915 | CID 224,919 | CID 224,926 | CID 224,930 | CID 224,931 |
| CID 225,717 | CID 225,790 | CID 225,792 | CID 225,794 | CID 225,795 | CID 225,796 |
| CID 225,805 | CID 226,567 | CID 227,258 | CID 240,550 | CID 271,854 | CID 279,138 |
| CID 279,142 | CID 279,144 | CID 279,147 | CID 284,456 | CID 299,579 | CID 326,416 |
| CID 408,518 | CID 408,544 | CID 408,739 | CID 408,759 | CID 411,587 | CID 420,950 |
| CID 420,951 | CID 420,952 | CID 420,953 | CID 420,954 | CID 420,955 | CID 428,747 |
| CID 432,515 | CID 436,771 | CID 456,326 | CID 2,751,968 | CID 2,777,263 | CID 3,032,648 |
| CID 3,081,396 | CID 3,246,048 | CID 3,695,004 | CID 3,868,826 | CID 3,891,403 | CID 3,959,885 |
| CID 4,349,542 | CID 4,591,706 | CID 5,027,238 | CID 5,231,914 | CID 5,246,643 | CID 5,311,441 |
| CID 5,351,653 | CID 5,354,627 | CID 5,362,453 | CID 5,463,897 | CID 5,464,034 | CID 5,464,322 |
| CID 5,464,511 | CID 5,771,529 | CID 6,093,409 | CID 6,326,778 | CID 6,327,011 | CID 6,328,579 |
| CID 6,328,716 | CID 6,328,740 | CID 6,331,889 | CID 6,331,890 | CID 6,331,891 | CID 6,331,895 |
| CID 6,331,896 | CID 6,331,897 | CID 6,331,898 | CID 6,331,899 | CID 6,331,900 | CID 6,331,902 |
| CID 6,331,903 | CID 6,331,905 | CID 6,332,020 | CID 6,333,908 | CID 6,333,910 | CID 6,335,289 |
| CID 6,338,392 | CID 6,367,296 | CID 6,367,298 | CID 6,368,256 | CID 6,373,292 | CID 6,374,203 |
| CID 6,377,210 | CID 6,377,218 | CID 6,383,299 | CID 6,383,671 | CID 6,386,841 | CID 6,387,196 |
| CID 6,390,184 | CID 6,390,186 | CID 6,392,009 | CID 6,392,668 | CID 6,393,991 | CID 6,394,511 |
| CID 6,304,680 | CID 6,394,681 | CID 6,395,057 | CID 6,395,059 | CID 6,396,115 | CID 6,396,418 |
| CID 6,396,421 | CID 6,396,442 | CID 6,396,719 | CID 6,396,810 | CID 6,397,382 | CID 6,397,391 |
| CID 6,397,717 | CID 6,398,204 | CID 6,857,634 | CID 6,857,636 | CID 9,810,717 | CID 9,885,488 |
| CID 9,915,631 | CID 10,090,090 | CID 10,090,091 | CID 10,259,654 | CID 10,939,670 | CID 10,960,282 |
| CID 10,960,305 | CID 11,005,470 | CID 11,118,066 | CID 11,297,036 | CID 11,318,239 | CID 11,730,658 |
| CID 11,765,685 | CID 11,768,112 | CID 11,807,053 | CID 11,966,274 | CID 11,966,275 | CID 11,980,764 |
| CID 11,985,984 | CID 12,591,815 | CID 12,725,709 | CID 13,047,299 | CID 13,467,894 | CID 13,678,820 |
| CID 13,678,826 | CID 13,751,471 | CID 13,783,183 | CID 13,788,547 | CID 13,956,203 | CID 14,085,961 |
| CID 14,324,919 | CID 15,238,731 | CID 15,388,575 | CID 15,396,527 | CID 15,531,077 | CID 15,531,078 |
| CID 15,829,268 | CID 15,845,290 | CID 15,951,499 | CID 16,132,666 | CID 16,132,670 | CID 16,132,838 |
| CID 16,132,942 | CID 16,133,190 | CID 16,133,363 | CID 16,133,379 | CID 16,211,378 | CID 16,211,954 |
| CID 16,213,713 | CID 16,656,342 | CID 16,683,012 | CID 16,683,013 | CID 16,683,083 | CID 16,683,100 |
| CID 16,683,108 | CID 16,683,119 | CID 16,683,598 | CID 16,683,599 | CID 16,683,601 | CID 16,683,608 |
| CID 16,683,609 | CID 16,683,611 | CID 16,683,613 | CID 16,683,614 | CID 16,683,616 | CID 16,683,618 |
| CID 16,683,620 | CID 16,683,621 | CID 16,683,623 | CID 16,683,653 | CID 16,683,881 | CID 16,683,992 |
| CID 16,684,366 | CID 16,684,410 | CID 16,684,725 | CID 16,684,726 | CID 16,684,730 | CID 16,685,150 |
| CID 16,685,183 | CID 16,685,196 | CID 16,685,245 | CID 16,685,271 | CID 16,685,394 | CID 16,685,395 |
| CID 16,685,549 | CID 16,685,550 | CID 16,685,551 | CID 16,685,552 | CID 16,685,588 | CID 16,685,683 |
| CID 16,685,687 | CID 16,685,688 | CID 16,686,075 | CID 16,686,100 | CID 16,686,112 | CID 16,686,186 |
| CID 16,686,196 | CID 16,686,225 | CID 16,686,229 | CID 16,686,232 | CID 16,686,233 | CID 16,686,253 |
| CID 16,686,296 | CID 16,686,298 | CID 16,686,321 | CID 16,686,393 | CID 16,686,395 | CID 16,686,397 |
| CID 16,686,399 | CID 16,686,401 | CID 16,686,432 | CID 16,686,484 | CID 16,686,517 | CID 16,686,548 |
| CID 16,686,570 | CID 16,686,603 | CID 16,686,636 | CID 16,686,659 | CID 16,686,663 | CID 16,686,674 |
| CID 16,686,677 | CID 16,686,683 | CID 16,686,685 | CID 16,686,688 | CID 16,686,700 | CID 16,686,707 |
| CID 16,686,709 | CID 16,686,711 | CID 16,686,714 | CID 16,686,716 | CID 16,686,719 | CID 16,686,722 |
| CID 16,686,723 | CID 16,686,725 | CID 16,686,730 | CID 16,686,754 | CID 16,686,756 | CID 16,686,763 |
| CID 16,686,767 | CID 16,686,789 | CID 16,686,792 | CID 16,686,794 | CID 16,686,796 | CID 16,686,798 |
| CID 16,686,802 | CID 16,686,808 | CID 16,686,810 | CID 16,686,812 | CID 16,686,816 | CID 16,686,830 |
| CID 16,686,841 | CID 16,686,843 | CID 16,686,846 | CID 16,686,848 | CID 16,686,851 | CID 16,686,855 |
| CID 16,686,860 | CID 16,686,870 | CID 16,686,878 | CID 16,686,890 | CID 16,686,893 | CID 16,686,895 |
| CID 16,686,898 | CID 16,686,900 | CID 16,686,904 | CID 16,686,907 | CID 16,686,909 | CID 16,686,911 |
| CID 16,686,913 | CID 16,686,917 | CID 16,686,921 | CID 16,686,928 | CID 16,686,930 | CID 16,686,932 |
| CID 16,686,937 | CID 16,686,939 | CID 16,686,941 | CID 16,686,943 | CID 16,686,948 | CID 16,686,950 |
| CID 16,686,952 | CID 16,686,954 | CID 16,686,957 | CID 16,686,959 | CID 16,686,968 | CID 16,686,977 |
| CID 16,686,979 | CID 16,686,982 | CID 16,686,983 | CID 16,686,986 | CID 16,686,988 | CID 16,686,991 |
| CID 16,687,010 | CID 16,687,016 | CID 16,687,021 | CID 16,687,023 | CID 16,687,026 | CID 16,687,029 |
| CID 16,687,034 | CID 16,687,038 | CID 16,687,043 | CID 16,687,044 | CID 16,687,053 | CID 16,687,055 |
| CID 16,687,059 | CID 16,687,061 | CID 16,687,063 | CID 16,687,066 | CID 16,687,069 | CID 16,687,071 |
| CID 16,687,099 | CID 16,687,102 | CID 16,687,107 | CID 16,687,110 | CID 16,687,112 | CID 16,687,116 |
| CID 16,687,119 | CID 16,687,121 | CID 16,687,125 | CID 16,687,140 | CID 16,687,142 | CID 16,687,144 |
| CID 16,687,148 | CID 16,687,151 | CID 16,687,155 | CID 16,687,166 | CID 16,687,174 | CID 16,687,177 |
| CID 16,687,180 | CID 16,687,183 | CID 16,687,185 | CID 16,687,187 | CID 16,687,189 | CID 16,687,191 |
| CID 16,687,218 | CID 16,687,220 | CID 16,687,222 | CID 16,687,224 | CID 16,687,226 | CID 16,687,232 |
| CID 16,687,234 | CID 16,687,256 | CID 16,687,259 | CID 16,687,261 | CID 16,687,263 | CID 16,687,268 |
| CID 16,687,286 | CID 16,687,298 | CID 16,687,306 | CID 16,687,332 | CID 16,687,334 | CID 16,687,339 |
| CID 16,687,354 | CID 16,687,371 | CID 16,687,376 | CID 16,687,390 | CID 16,687,408 | CID 16,687,417 |
| CID 16,687,458 | CID 16,687,461 | CID 16,687,464 | CID 16,687,465 | CID 16,687,471 | CID 16,687,475 |
| CID 16,687,482 | CID 16,687,483 | CID 16,687,518 | CID 16,687,546 | CID 16,687,556 | CID 16,687,558 |
| CID 16,687,566 | CID 16,687,573 | CID 16,687,608 | CID 16,687,610 | CID 16,687,630 | CID 16,687,631 |
| CID 16,687,633 | CID 16,687,642 | CID 16,687,648 | CID 16,687,649 | CID 16,687,666 | CID 16,687,668 |
| CID 16,687,675 | CID 16,687,677 | CID 16,687,684 | CID 16,687,688 | CID 16,687,717 | CID 16,687,722 |
| CID 16,687,726 | CID 16,687,741 | CID 16,687,743 | CID 16,687,765 | CID 16,687,796 | CID 16,687,800 |
| CID 16,687,815 | CID 16,687,828 | CID 16,687,833 | CID 16,687,837 | CID 16,687,872 | CID 16,687,875 |
| CID 16,687,876 | CID 16,687,894 | CID 16,687,898 | CID 16,687,912 | CID 16,687,918 | CID 16,687,923 |
| CID 16,687,925 | CID 16,687,931 | CID 16,687,934 | CID 16,687,936 | CID 16,687,940 | CID 16,687,962 |
| CID 16,687,968 | CID 16,687,972 | CID 16,687,982 | CID 16,687,985 | CID 16,687,990 | CID 16,688,008 |
| CID 16,688,015 | CID 16,688,017 | CID 16,688,019 | CID 16,688,023 | CID 16,688,027 | CID 16,688,032 |
| CID 16,688,037 | CID 16,688,044 | CID 16,688,052 | CID 16,688,060 | CID 16,688,062 | CID 16,688,085 |
| CID 16,688,119 | CID 16,688,127 | CID 16,688,128 | CID 16,688,132 | CID 16,688,150 | CID 16,688,155 |
| CID 16,688,161 | CID 16,688,166 | CID 16,688,171 | CID 16,688,175 | CID 16,688,181 | CID 16,688,199 |
| CID 16,688,203 | CID 16,688,204 | CID 16,688,213 | CID 16,688,236 | CID 16,688,237 | CID 16,688,238 |
| CID 16,688,242 | CID 16,688,246 | CID 16,688,273 | CID 16,688,277 | CID 16,688,296 | CID 16,688,304 |
| CID 16,688,309 | CID 16,688,313 | CID 16,688,340 | CID 16,688,344 | CID 16,688,345 | CID 16,688,347 |
| CID 16,688,354 | CID 16,688,358 | CID 16,688,360 | CID 16,688,361 | CID 16,688,378 | CID 16,688,385 |
| CID 16,688,391 | CID 16,688,396 | CID 16,688,400 | CID 16,688,451 | CID 16,688,470 | CID 16,688,480 |
| CID 16,688,481 | CID 16,688,483 | CID 16,688,490 | CID 16,688,492 | CID 16,688,502 | CID 16,688,509 |
| CID 16,688,511 | CID 16,688,513 | CID 16,688,514 | CID 16,688,522 | CID 16,688,529 | CID 16,688,534 |
| CID 16,688,538 | CID 16,688,546 | CID 16,688,549 | CID 16,688,551 | CID 16,688,564 | CID 16,688,571 |
| CID 16,688,572 | CID 16,688,581 | CID 16,688,584 | CID 16,688,600 | CID 16,688,605 | CID 16,688,607 |
| CID 16,688,612 | CID 16,688,623 | CID 16,688,630 | CID 16,688,633 | CID 16,688,640 | CID 16,688,641 |
| CID 16,688,650 | CID 16,688,674 | CID 16,688,678 | CID 16,688,722 | CID 16,688,726 | CID 16,688,729 |
| CID 16,688,731 | CID 16,688,735 | CID 16,688,740 | CID 16,688,742 | CID 16,688,744 | CID 16,688,746 |
| CID 16,688,769 | CID 16,688,787 | CID 16,688,789 | CID 16,688,793 | CID 16,688,804 | CID 16,688,811 |
| CID 16,688,822 | CID 16,688,826 | CID 16,688,828 | CID 16,688,834 | CID 16,688,841 | CID 16,688,854 |
| CID 16,688,859 | CID 16,688,866 | CID 16,688,871 | CID 16,688,873 | CID 16,688,874 | CID 16,688,875 |
| CID 16,688,888 | CID 16,688,899 | CID 16,688,903 | CID 16,688,904 | CID 16,688,907 | CID 16,688,910 |
| CID 16,688,911 | CID 16,688,954 | CID 16,688,968 | CID 16,688,976 | CID 16,689,000 | CID 16,689,003 |
| CID 16,689,020 | CID 16,689,026 | CID 16,689,028 | CID 16,689,030 | CID 16,689,032 | CID 16,689,034 |
| CID 16,689,035 | CID 16,689,039 | CID 16,689,043 | CID 16,689,066 | CID 16,689,069 | CID 16,689,074 |
| CID 16,689,091 | CID 16,689,122 | CID 16,689,148 | CID 16,689,159 | CID 16,689,180 | CID 16,689,193 |
| CID 16,689,197 | CID 16,689,229 | CID 16,689,244 | CID 16,689,275 | CID 16,689,282 | CID 16,689,288 |
| CID 16,689,289 | CID 16,689,296 | CID 16,689,306 | CID 16,689,321 | CID 16,689,323 | CID 16,689,331 |
| CID 16,689,333 | CID 16,689,362 | CID 16,689,378 | CID 16,689,389 | CID 16,689,407 | CID 16,689,423 |
| CID 16,689,425 | CID 16,689,428 | CID 16,689,429 | CID 16,689,433 | CID 16,689,435 | CID 16,689,438 |
| CID 16,689,447 | CID 16,689,486 | CID 16,689,496 | CID 16,689,500 | CID 16,689,506 | CID 16,689,509 |
| CID 16,689,515 | CID 16,689,518 | CID 16,689,523 | CID 16,689,524 | CID 16,689,527 | CID 16,689,520 |
| CID 16,689,562 | CID 16,689,563 | CID 16,689,564 | CID 16,689,565 | CID 16,689,571 | CID 16,689,573 |
| CID 16,689,584 | CID 16,689,586 | CID 16,689,611 | CID 16,689,613 | CID 16,689,622 | CID 16,689,627 |
| CID 16,689,630 | CID 16,689,632 | CID 16,689,635 | CID 16,689,637 | CID 16,689,640 | CID 16,689,647 |
| CID 16,689,663 | CID 16,689,666 | CID 16,689,673 | CID 16,689,678 | CID 16,689,683 | CID 16,689,685 |
| CID 16,689,691 | CID 16,689,695 | CID 16,689,727 | CID 16,689,729 | CID 16,689,736 | CID 16,689,738 |
| CID 16,689,740 | CID 16,689,741 | CID 16,689,753 | CID 16,689,755 | CID 16,689,758 | CID 16,689,760 |
| CID 16,690,353 | CID 16,692,259 | CID 16,693,842 | CID 16,693,976 | CID 16,694,718 | CID 16,694,762 |
| CID 16,695,738 | CID 16,696,653 | CID 16,696,654 | CID 16,696,933 | CID 16,697,139 | CID 16,697,406 |
| CID 16,697,408 | CID 16,697,957 | CID 16,697,959 | CID 16,697,961 | CID 16,698,179 | CID 16,699,328 |
| CID 16,699,521 | CID 16,700,362 | CID 16,700,586 | CID 16,700,907 | CID 16,700,908 | CID 16,701,189 |
| CID 16,701,408 | CID 16,701,417 | CID 16,701,539 | CID 16,702,362 | CID 16,702,435 | CID 16,703,268 |
| CID 16,703,361 | CID 16,703,710 | CID 16,704,622 | CID 16,705,392 | CID 16,705,462 | CID 16,705,463 |
| CID 16,705,466 | CID 16,705,531 | CID 16,705,533 | CID 16,705,535 | CID 16,705,545 | CID 16,705,675 |
| CID 16,706,113 | CID 16,706,116 | CID 16,706,118 | CID 16,706,119 | CID 16,706,769 | CID 16,707,877 |
| CID 16,707,993 | CID 16,708,818 | CID 16,710,497 | CID 16,710,763 | CID 16,711,795 | CID 16,711,797 |
| CID 16,715,788 | CID 16,715,938 | CID 16,717,102 | CID 16,717,107 | CID 16,717,535 | CID 16,717,578 |
| CID 16,717,583 | CID 16,717,592 | CID 16,717,593 | CID 16,717,595 | CID 16,717,603 | CID 16,717,605 |
| CID 16,717,643 | CID 16,717,644 | CID 16,717,652 | CID 16,717,657 | CID 16,717,678 | CID 16,726,724 |
| CID 16,726,725 | CID 16,726,928 | CID 16,727,069 | CID 16,742,676 | CID 16,750,698 | CID 16,750,700 |
| CID 16,750,702 | CID 16,750,704 | CID 16,750,706 | CID 16,751,742 | CID 16,752,035 | CID 16,752,037 |
| CID 16,752,039 | CID 16,752,157 | CID 16,752,159 | CID 16,752,280 | CID 16,752,282 | CID 16,752,284 |
| CID 16,752,384 | CID 16,752,386 | CID 16,752,388 | CID 16,752,390 | CID 16,752,503 | CID 16,752,505 |
| CID 16,752,507 | CID 16,752,509 | CID 16,760,656 | CID 17,757,229 | CID 17,757,914 | CID 17,764,892 |
| CID 17,778,956 | CID 17,778,957 | CID 17,786,555 | CID 17,786,556 | CID 17,817,953 | CID 17,822,508 |
| CID 17,822,510 | CID 17,826,684 | CID 17,849,914 | CID 17,884,946 | CID 17,897,207 | CID 17,917,039 |
| CID 17,936,221 | CID 17,945,298 | CID 17,964,729 | CID 17,976,351 | CID 18,000,152 | CID 18,006,734 |
| CID 18,182,969 | CID 18,350,096 | CID 18,350,179 | CID 18,350,195 | CID 18,350,251 | CID 18,350,260 |
| CID 18,350,272 | CID 18,352,342 | CID 18,352,411 | CID 18,352,412 | CID 18,362,431 | CID 18,364,093 |
| CID 18,364,110 | CID 18,369,789 | CID 18,377,198 | CID 18,387,057 | CID 18,387,058 | CID 18,387,060 |
| CID 18,387,072 | CID 18,401,200 | CID 18,458,528 | CID 18,458,565 | CID 18,468,794 | CID 18,502,979 |
| CID 18,503,509 | CID 18,503,583 | CID 18,503,597 | CID 18,503,607 | CID 18,503,614 | CID 18,503,626 |
| CID 18,503,641 | CID 18,503,668 | CID 18,503,674 | CID 18,503,676 | CID 18,503,678 | CID 18,503,713 |
| CID 18,503,717 | CID 18,503,718 | CID 18,503,720 | CID 18,503,730 | CID 18,503,732 | CID 18,503,745 |
| CID 18,503,756 | CID 18,503,760 | CID 18,503,762 | CID 18,503,780 | CID 18,503,786 | CID 18,503,788 |
| CID 18,503,795 | CID 18,503,809 | CID 18,503,817 | CID 18,503,829 | CID 18,503,834 | CID 18,503,854 |
| CID 18,503,855 | CID 18,503,866 | CID 18,513,362 | CID 18,513,368 | CID 18,513,395 | CID 18,517,340 |
| CID 18,517,342 | CID 18,517,343 | CID 18,533,808 | CID 18,619,949 | CID 18,620,401 | CID 18,698,857 |
| CID 18,698,858 | CID 18,698,859 | CID 18,698,860 | CID 18,719,776 | CID 18,755,853 | CID 18,759,325 |
| CID 18,764,251 | CID 18,764,466 | CID 18,794,972 | CID 18,801,121 | CID 18,801,139 | CID 18,931,796 |
| CID 18,951,198 | CID 18,954,064 | CID 18,963,165 | CID 18,968,263 | CID 18,972,203 | CID 18,981,572 |
| CID 18,982,367 | CID 18,982,494 | CID 18,982,517 | CID 18,986,400 | CID 18,986,401 | CID 19,019,300 |
| CID 19,042,246 | CID 19,044,994 | CID 19,045,026 | CID 19,068,398 | CID 19,073,968 | CID 19,073,976 |
| CID 19,083,438 | CID 19,093,335 | CID 19,097,031 | CID 19,097,032 | CID 19,097,034 | CID 19,097,036 |
| CID 19,347,784 | CID 19,349,306 | CID 19,351,976 | CID 19,354,077 | CID 19,366,336 | CID 19,366,341 |
| CID 19,366,346 | CID 19,366,352 | CID 19,372,168 | CID 19,372,178 | CID 19,373,039 | CID 19,373,841 |
| CID 19,373,847 | CID 19,373,849 | CID 19,379,549 | CID 19,417,354 | CID 19,432,230 | CID 19,594,006 |
| CID 19,594,014 | CID 19,594,015 | CID 19,594,016 | CID 19,609,633 | CID 19,698,871 | CID 19,700,212 |
| CID 19,734,742 | CID 19,734,744 | CID 19,734,745 | CID 19,739,452 | CID 19,748,302 | CID 19,762,749 |
| CID 19,762,750 | CID 19,762,751 | CID 19,762,752 | CID 19,762,753 | CID 19,762,755 | CID 19,774,966 |
| CID 19,795,275 | CID 19,795,276 | CID 19,795,277 | CID 19,795,280 | CID 19,795,286 | CID 19,795,293 |
| CID 19,795,298 | CID 19,795,302 | CID 19,821,446 | CID 19,840,059 | CID 19,877,071 | CID 19,887,685 |
| CID 19,887,689 | CID 19,887,691 | CID 19,887,696 | CID 19,887,720 | CID 19,887,725 | CID 19,899,974 |
| CID 19,913,507 | CID 19,969,014 | CID 19,969,017 | CID 19,969,018 | CID 19,969,019 | CID 19,969,021 |
| CID 19,969,022 | CID 19,969,023 | CID 19,969,024 | CID 19,969,026 | CID 19,969,027 | CID 19,969,031 |
| CID 19,969,032 | CID 19,969,036 | CID 19,969,037 | CID 19,969,039 | CID 19,969,040 | CID 19,969,041 |
| CID 19,969,042 | CID 19,969,043 | CID 19,971,369 | CID 19,975,042 | CID 19,981,557 | CID 19,983,424 |
| CID 19,991,813 | CID 19,993,400 | CID 19,993,402 | CID 19,993,412 | CID 19,993,418 | CID 20,038,203 |
| CID 20,056,484 | CID 20,056,601 | CID 20,063,739 | CID 20,063,745 | CID 20,083,778 | CID 20,084,143 |
| CID 20,084,144 | CID 20,084,145 | CID 20,146,627 | CID 20,146,628 | CID 20,186,109 | CID 20,195,292 |
| CID 20,235,371 | CID 20,259,845 | CID 20,291,586 | CID 20,291,592 | CID 20,314,491 | CID 20,391,574 |
| CID 20,401,169 | CID 20,443,249 | CID 20,443,251 | CID 20,463,996 | CID 20,474,401 | CID 20,474,407 |
| CID 20,474,432 | CID 20,474,444 | CID 20,493,178 | CID 20,529,305 | CID 20,529,307 | CID 20,529,309 |
| CID 20,549,661 | CID 20,554,899 | CID 20,562,912 | CID 20,658,850 | CID 20,669,215 | CID 20,670,558 |
| CID 20,835,778 | CID 20,835,967 | CID 20,836,032 | CID 20,836,033 | CID 20,836,037 | CID 20,836,063 |
| CID 20,836,064 | CID 20,836,065 | CID 20,836,066 | CID 20,836,067 | CID 20,836,069 | CID 20,841,651 |
| CID 20,846,195 | CID 20,846,196 | CID 20,981,344 | CID 20,981,345 | CID 20,981,346 | CID 20,981,347 |
| CID 21,096,833 | CID 21,113,988 | CID 21,119,588 | CID 21,126,179 | CID 21,127,108 | CID 21,127,109 |
| CID 21,139,532 | CID 21,139,533 | CID 21,188,886 | CID 21,188,887 | CID 21,188,889 | CID 21,188,890 |
| CID 21,201,179 | CID 21,263,248 | CID 21,287,599 | CID 21,290,433 | CID 21,292,545 | CID 21,292,548 |
| CID 21,523,241 | CID 21,536,746 | CID 21,646,515 | CID 21,646,518 | CID 21,646,542 | CID 21,646,556 |
| CID 21,646,561 | CID 21,646,575 | CID 21,649,357 | CID 21,685,678 | CID 21,732,577 | CID 21,732,578 |
| CID 21,732,579 | CID 21,737,067 | CID 21,756,111 | CID 21,840,658 | CID 21,853,879 | CID 21,853,881 |
| CID 21,853,883 | CID 21,863,617 | CID 21,863,626 | CID 21,863,638 | CID 21,863,662 | CID 21,881,458 |
| CID 21,881,691 | CID 21,881,814 | CID 21,889,414 | CID 21,893,958 | CID 21,900,090 | CID 21,902,214 |
| CID 21,903,750 | CID 21,903,777 | CID 21,924,639 | CID 21,924,665 | CID 21,953,576 | CID 21,964,863 |
| CID 21,972,194 | CID 21,972,202 | CID 21,982,758 | CID 21,987,541 | CID 21,989,411 | CID 21,993,039 |
| CID 21,993,041 | CID 21,993,046 | CID 21,996,680 | CID 21,996,687 | CID 21,996,691 | CID 21,996,700 |
| CID 21,996,702 | CID 21,996,706 | CID 21,996,719 | CID 22,005,423 | CID 22,005,428 | CID 22,005,432 |
| CID 22,020,061 | CID 22,020,062 | CID 22,020,065 | CID 22,024,009 | CID 22,035,134 | CID 22,053,046 |
| CID 22,053,047 | CID 22,056,400 | CID 22,056,405 | CID 22,058,812 | CID 22,073,265 | CID 22,116,673 |
| CID 22,119,339 | CID 22,119,529 | CID 22,129,262 | CID 22,131,377 | CID 22,168,435 | CID 22,227,347 |
| CID 22,228,659 | CID 22,228,668 | CID 22,228,669 | CID 22,229,092 | CID 22,234,749 | CID 22,239,852 |
| CID 22,245,544 | CID 22,257,403 | CID 22,257,408 | CID 22,257,416 | CID 22,257,444 | CID 22,266,542 |
| CID 22,311,743 | CID 22,327,955 | CID 22,342,067 | CID 22,342,072 | CID 22,342,089 | CID 22,342,093 |
| CID 22,342,111 | CID 22,342,138 | CID 22,342,145 | CID 22,342,158 | CID 22,342,172 | CID 22,342,180 |
| CID 22,342,188 | CID 22,342,193 | CID 22,342,196 | CID 22,342,197 | CID 22,342,212 | CID 22,342,213 |
| CID 22,342,223 | CID 22,342,226 | CID 22,342,228 | CID 22,342,235 | CID 22,342,237 | CID 22,342,243 |
| CID 22,342,246 | CID 22,342,251 | CID 22,342,260 | CID 22,342,268 | CID 22,342,271 | CID 22,348,440 |
| CID 22,348,441 | CID 22,373,310 | CID 22,382,064 | CID 22,395,621 | CID 22,416,288 | CID 22,476,867 |
| CID 22,476,868 | CID 22,476,869 | CID 22,476,870 | CID 22,476,873 | CID 22,476,877 | CID 22,559,686 |
| CID 22,593,186 | CID 22,593,461 | CID 22,597,237 | CID 22,617,368 | CID 22,619,535 | CID 22,619,538 |
| CID 22,619,540 | CID 22,619,543 | CID 22,619,546 | CID 22,619,548 | CID 22,619,550 | CID 22,619,556 |
| CID 22,619,560 | CID 22,619,562 | CID 22,619,563 | CID 22,619,565 | CID 22,619,568 | CID 22,619,570 |
| CID 22,619,572 | CID 22,619,575 | CID 22,619,577 | CID 22,619,579 | CID 22,619,583 | CID 22,619,586 |
| CID 22,619,595 | CID 22,619,597 | CID 22,619,599 | CID 22,619,601 | CID 22,619,605 | CID 22,619,610 |
| CID 22,622,810 | CID 22,667,472 | CID 22,667,473 | CID 22,677,101 | CID 22,677,105 | CID 22,677,107 |
| CID 22,712,891 | CID 22,718,954 | CID 22,721,302 | CID 22,724,594 | CID 22,726,239 | CID 22,741,572 |
| CID 22,741,573 | CID 22,741,582 | CID 22,836,337 | CID 22,924,281 | CID 22,924,283 | CID 22,930,289 |
| CID 22,944,824 | CID 22,987,297 | CID 22,988,973 | CID 22,988,978 | CID 22,988,989 | CID 22,988,998 |
| CID 22,989,008 | CID 22,989,019 | CID 22,989,021 | CID 22,989,034 | CID 22,989,043 | CID 22,989,045 |
| CID 22,989,047 | CID 22,996,790 | CID 22,996,913 | CID 22,996,914 | CID 22,996,922 | CID 22,998,772 |
| CID 23,034,978 | CID 23,035,001 | CID 23,069,406 | CID 23,069,577 | CID 23,106,338 | CID 23,132,928 |
| CID 23,132,939 | CID 23,132,954 | CID 23,132,960 | CID 23,132,976 | CID 23,132,994 | CID 23,133,066 |
| CID 23,133,085 | CID 23,133,101 | CID 23,133,103 | CID 23,133,111 | CID 23,133,135 | CID 23,133,208 |
| CID 23,133,220 | CID 23,133,233 | CID 23,133,240 | CID 23,133,243 | CID 23,158,823 | CID 23,172,887 |
| CID 23,173,756 | CID 23,234,545 | CID 23,237,442 | CID 23,237,443 | CID 23,237,638 | CID 23,237,639 |
| CID 23,237,640 | CID 23,263,168 | CID 23,290,347 | CID 23,290,357 | CID 23,290,626 | CID 23,297,456 |
| CID 23,297,457 | CID 23,297,458 | CID 23,297,460 | CID 23,297,461 | CID 23,349,105 | CID 23,358,537 |
| CID 23,364,976 | CID 23,368,821 | CID 23,434,970 | CID 23,447,009 | CID 23,452,070 | CID 23,452,071 |
| CID 23,496,438 | CID 23,505,292 | CID 23,622,502 | CID 23,622,503 | CID 23,626,513 | CID 23,626,673 |
| CID 23,626,677 | CID 23,626,679 | CID 23,626,680 | CID 23,626,842 | CID 23,626,844 | CID 23,627,000 |
| CID 23,627,001 | CID 23,627,004 | CID 23,627,005 | CID 23,627,157 | CID 23,627,158 | CID 23,630,531 |
| CID 23,633,149 | CID 23,638,203 | CID 23,638,567 | CID 23,659,334 | CID 23,665,040 | CID 23,667,254 |
| CID 23,667,270 | CID 23,667,271 | CID 23,669,627 | CID 23,667,988 | CID 23,678,227 | CID 23,686,987 |
| CID 23,686,988 | CID 23,686,989 | CID 23,695,002 | CID 23,700,084 | CID 23,715,661 | CID 23,716,568 |
| CID 23,716,909 | CID 23,719,542 | CID 23,719,543 | CID 24,182,112 | CID 24,182,113 | CID 24,182,140 |
| CID 24,182,326 | CID 24,182,329 | CID 24,182,333 | CID 24,182,335 | CID 24,182,336 | CID 24,182,337 |
| CID 24,182,338 | CID 24,182,339 | CID 24,182,340 | CID 24,182,341 | CID 24,182,342 | CID 24,182,343 |
| CID 24,183,194 | CID 24,204,782 | CID 24,204,783 | CID 24,204,784 | CID 24,204,785 | CID 24,741,041 |
| CID 24,756,103 | CID 24,798,913 | CID 24,809,138 | CID 24,867,560 | CID 24,884,187 | CID 24,906,211 |
| CID 25,022,171 | CID 25,076,497 | CID 25,076,666 | CID 25,193,242 | CID 25,194,794 | CID 25,194,796 |
| CID 25,194,798 | CID 25,228,294 | CID 42,643,355 | CID 44,148,022 | CID 44,153,142 | CID 44,153,290 |
| CID 44,228,599 | CID 44,233,597 | CID 44,238,265 | CID 44,238,631 | CID 44,239,672 | CID 44,240,267 |
| CID 44,249,223 | CID 44,512,541 | CID 44,512,542 | CID 44,517,919 | CID 44,517,925 | CID 44,541,942 |
| CID 44,597,116 | CID 44,717,411 | CID 44,717,411 | CID 44,717,411 | CID 44,717,573 | CID 44,817,506 |
| CID 44,887,160 | CID 45,044,973 | CID 45,045,188 | CID 45,045,192 | CID 45,045,193 | CID 45,045,194 |
| CID 45,045,196 | CID 45,045,199 | CID 45,045,200 | CID 45,045,210 | CID 45,045,211 | CID 45,049,019 |
| CID 45,049,600 | CID 45,050,451 | CID 45,050,453 | CID 45,357,504 | CID 45,480,132 | CID 45,933,604 |
| CID 45,933,659 | CID 46,183,771 | CID 46,188,363 | CID 46,189,690 | CID 46,237,239 | CID 46,872,310 |
| CID 46,899,649 | CID 46,928,906 | CID 46,928,909 | CID 46,929,660 | CID 46,929,662 | CID 46,929,664 |
| CID 46,929,767 | CID 46,929,769 | CID 46,929,771 | CID 46,929,773 | CID 46,929,775 | CID 46,929,877 |
| CID 46,292,879 | CID 46,929,881 | CID 46,929,883 | CID 46,929,885 | CID 46,929,983 | CID 46,929,985 |
| CID 46,929,987 | CID 46,929,989 | CID 46,929,991 | CID 46,930,089 | CID 46,930,738 | CID 46,930,740 |
| CID 46,930,742 | CID 46,930,744 | CID 46,930,832 | CID 46,930,834 | CID 46,930,836 | CID 46,930,838 |
| CID 46,930,840 | CID 46,930,842 | CID 46,930,931 | CID 46,930,933 | CID 46,930,935 | CID 49,793,478 |
| CID 49,836,322 | CID 49,853,493 | CID 49,874,344 | CID 49,874,345 | CID 49,874,346 | CID 50,905,223 |
| CID 50,905,225 | CID 50,905,228 | CID 50,905,230 | CID 50,907,557 | CID 50,907,559 | CID 50,907,773 |
| CID 50,907,775 | CID 50,907,777 | CID 50,907,779 | CID 50,908,230 | CID 50,908,463 | CID 50,908,465 |
| CID 50,908,469 | CID 50,908,470 | CID 50,911,248 | CID 50,911,456 | CID 50,933,198 | CID 50,934,395 |
| CID 50,942,467 | CID 50,942,549 | CID 51,031,263 | CID 51,050,603 | CID 52,951,063 | CID 52,952,662 |
| CID 53,297,348 | CID 53,443,043 | CID 53,465,423 | CID 53,469,680 | CID 53,471,861 | CID 54,599,893 |
| CID 54,600,651 | CID 54,602,403 | CID 54,605,141 | CID 54,605,327 | CID 54,607,458 | CID 54,607,459 |
| CID 54,607,892 | CID 54,609,460 | CID 54,609,461 | CID 54,690,107 | CID 54,690,110 | CID 54,690,112 |
| CID 56,603,832 | CID 56,641,575 | CID 56,642,813 | CID 56,642,814 | CID 56,642,815 | CID 56,649,287 |
| CID 56,649,288 | CID 56,649,289 | CID 56,649,290 | CID 56,649,291 | CID 56,649,292 | CID 56,649,293 |
| CID 56,650,053 | CID 56,838,736 | CID 56,842,480 | CID 56,927,674 | CID 56,931,004 | CID 56,954,063 |
| CID 57,347,420 | CID 57,348,099 | CID 57,348,199 | CID 57,348,242 | CID 57,350,786 | CID 57,353,046 |
| CID 57,369,581 | CID 57,369,582 | CID 57,370,240 | CID 57,371,214 | CID 57,404,131 | CID 57,404,132 |
| CID 57,404,141 | CID 57,404,144 | CID 57,479,380 | CID 57,479,381 | CID 57,801,122 | CID 57,801,123 |
| CID 58,271,550 | CID 58,271,568 | CID 58,271,571 | CID 58,708,617 | CID 58,954,727 | CID 58,981,248 |
| CID 59,423,141 | CID 59,469,260 | CID 59,862,225 | CID 59,876,870 | CID 59,941,562 | CID 59,945,033 |
| CID 59,945,049 | CID 59,953,892 | CID 60,153,069 | CID 60,203,006 | CID 70,682,647 | CID 71,295,992 |
| CID 71,300,790 | CID 71,301,085 | CID 71,301,086 | CID 71,301,087 | CID 71,301,088 | CID 71,301,100 |
| CID 71,301,433 | CID 71,306,778 | CID 71,306,778 | CID 71,306,979 | CID 71,311,282 | CID 71,312,650 |
| CID 71,340,449 | CID 71,345,114 | CID 71,361,359 | CID 71,372,842 | CID 71,430,991 | CID 71,434,327 |
| CID 71,434,328 | CID 71,447,128 | CID 71,475,865 | CID 71,479,473 | CID 71,500,253 | CID 71,517,948 |
| CID 71,528,218 | CID 71,578,862 | CID 71,658,082 | CID 71,732,632 | CID 71,732,634 | CID 71,741,547 |
| CID 72,164,734 | CID 72,203,693 | CID 72,736,046 | CID 72,736,768 | CID 72,941,503 | CID 73,212,144 |
| CID 73,307,636 | CID 73,555,299 | CID 73,555,452 | CID 73,555,495 | CID 73,556,098 | CID 73,557,134 |
| CID 73,780,045 | CID 73,894,307 | CID 73,894,317 | CID 73,894,318 | CID 73,894,319 | CID 74,082,032 |
| CID 74,412,563 | CID 76,963,945 | CID 77,620,826 | CID 84,819,435 | CID 85,524,452 | CID 85,770,041 |
| CID 85,787,724 | CID 85,896,371 | CID 85,896,374 | CID 85,972,541 | CID 86,154,368 | CID 86,600,076 |
| CID 86,600,078 | CID 86,600,080 | CID 86,629,112 | CID 86,638,468 | CID 86,638,469 | CID 86,638,470 |
| CID 86,642,991 | CID 86,664,723 | CID 86,738,015 | CID 86,745,982 | CID 86,745,983 | CID 86,745,984 |
| CID 86,748,578 | CID 86,748,579 | CID 86,755,560 | CID 87,109,675 | CID 87,148,513 | CID 87,261,616 |
| CID 87,261,618 | CID 87,261,627 | CID 87,261,632 | CID 87,261,634 | CID 87,261,636 | CID 87,261,653 |
| CID 87,261,659 | CID 87,261,710 | CID 87,261,723 | CID 87,261,725 | CID 87,261,733 | CID 87,261,734 |
| CID 87,261,738 | CID 87,261,740 | CID 87,261,750 | CID 87,261,751 | CID 87,261,753 | CID 87,261,754 |
| CID 87,261,758 | CID 87,273,685 | CID 87,315,251 | CID 87,315,420 | CID 87,315,759 | CID 87,357,341 |
| CID 87,357,342 | CID 87,372,967 | CID 87,439,741 | CID 87,475,080 | CID 87,475,225 | CID 87,477,991 |
| CID 87,574,556 | CID 87,574,667 | CID 87,575,582 | CID 87,575,818 | CID 87,575,929 | CID 87,625,935 |
| CID 87,684,551 | CID 87,684,552 | CID 87,702,250 | CID 87,740,953 | CID 87,743,651 | CID 87,886,395 |
| CID 87,930,618 | CID 87,932,050 | CID 87,950,566 | CID 87,973,266 | CID 88,095,364 | CID 88,096,059 |
| CID 88,104,535 | CID 88,157,706 | CID 88,176,249 | CID 88,198,568 | CID 88,222,470 | CID 88,254,467 |
| CID 88,258,496 | CID 88,296,023 | CID 88,374,845 | CID 88,413,526 | CID 88,435,151 | CID 88,445,516 |
| CID 88,445,518 | CID 88,473,380 | CID 88,473,411 | CID 88,473,488 | CID 88,473,759 | CID 88,492,068 |
| CID 88,492,070 | CID 88,499,769 | CID 88,607,097 | CID 88,619,303 | CID 88,622,678 | CID 88,624,465 |
| CID 88,624,466 | CID 88,626,735 | CID 88,638,060 | CID 88,643,234 | CID 88,689,628 | CID 88,701,486 |
| CID 88,735,730 | CID 88,738,422 | CID 88,747,108 | CID 88,749,053 | CID 88,749,265 | CID 88,749,279 |
| CID 88,749,282 | CID 88,760,630 | CID 88,760,633 | CID 88,760,980 | CID 88,775,758 | CID 88,804,969 |
| CID 90,471,465 | CID 90,472,492 | CID 90,472,811 | CID 90,473,364 | CID 90,473,686 | CID 90,476,658 |
| CID 90,476,658 | CID 90,477,046 | CID 90,478,732 | CID 90,659,536 | CID 90,661,669 | CID 91,654,628 |
| CID 91,666,506 | CID 91,666,539 | CID 91,668,039 | CID 91,668,040 | CID 91,867,184 | CID 91,868,044 |
| CID 91,868,424 | CID 91,886,666 | CID 91,979,898 | CID 91,996,051 | CID 91,996,299 | CID 91,997,848 |
| CID 92,004,796 | CID 92,026,277 | CID 92,028,425 | CID 92,028,431 | CID 92,029,717 | CID 92,043,174 |
| CID 102,600,862 | CID 102,601,227 | CID 102,601,620 | CID 102,602,550 | CID 117,064,703 | CID 117,064,732 |
| CID 117,064,750 | CID 117,064,773 | CID 117,064,814 | CID 117,064,943 | CID 117,065,015 | CID 117,065,049 |
| CID 117,065,281 | CID 117,065,286 | CID 117,065,352 | CID 117,065,353 | CID 117,094,805 | CID 118,885,762 |
| CID 118,856,320 | CID 119,025,724 | CID 119,075,307 | CID 119,075,410 | CID 119,075,411 | CID 119,075,412 |
| CID 119,075,413 | CID 119,075,414 | CID 121,225,415 | CID 121,233,523 | CID 121,235,197 | CID 121,513,981 |
| CID 122,129,633 | CID 122,129,634 | CID 122,404,843 | CID 122,409,375 | CID 124,219,876 | CID 127,262,626 |
| CID 127,262,627 | CID 129,627,836 | CID 129,627,837 | CID 129,628,445 | CID 129,631,374 | CID 129,631,549 |
| CID 129,631,550 | CID 129,632,304 | CID 129,636,318 | CID 129,637,112 | CID 129,637,113 | CID 129,640,894 |
| CID 129,655,685 | CID 129,655,962 | CID 129,656,034 | CID 129,656,045 | CID 129,656,052 | CID 129,663,025 |
| CID 129,663,276 | CID 129,664,704 | CID 129,664,993 | CID 129,666,672 | CID 129,666,688 | CID 129,666,712 |
| CID 129,666,726 | CID 129,672,591 | CID 129,672,604 | CID 129,672,608 | CID 129,672,616 | CID 129,677,390 |
| CID 129,677,407 | CID 129,678,204 | CID 129,681,485 | CID 129,681,498 | CID 129,681,530 | CID 129,684,391 |
| CID 124,684,392 | CID 129,691,988 | CID 129,692,842 | CID 129,708,928 | CID 129,723,561 | CID 129,738,338 |
| CID 129,741,653 | CID 129,760,359 | CID 129,762,840 | CID 129,772,466 | CID 129,784,023 | CID 129,794,466 |
| CID 129,801,784 | CID 129,801,784 | CID 129,801,784 | CID 129,809,167 | CID 129,809,204 | CID 129,809,234 |
| CID 129,809,241 | CID 129,809,273 | CID 129,809,286 | CID 129,809,383 | CID 129,815,113 | CID 129,815,572 |
| CID 129,815,618 | CID 129,815,644 | CID 129,815,671 | CID 129,816,899 | CID 129,821,553 | CID 129,821,554 |
| CID 129,826,505 | CID 129,827,852 | CID 129,851,725 | CID 129,852,375 | CID 129,875,229 | CID 129,885,650 |
| CID 129,890,416 | CID 129,893,591 | CID 129,893,718 | CID 129,896,656 | CID 131,635,452 | CID 131,697,748 |
| CID 131,707,326 | CID 131,707,326 | CID 131,712,430 | CID 131,713,084 | CID 131,719,336 | CID 131,731,322 |
| CID 131,738,151 | CID 131,739,790 | CID 131,739,792 | CID 131,842,086 | CID 131,842,288 | CID 131,845,848 |
| CID 131,870,833 | CID 131,873,231 | CID 131,876,773 | CID 131,878,506 | CID 131,880,760 | CID 131,883,481 |
| CID 131,883,482 | CID 131,883,905 | CID 131,883,906 | CID 131,884,069 | CID 131,884,369 | CID 131,884,370 |

TABLE 7
Exemplar PANDA Agents with structural and transcriptional activity rescue
verified by our experiments. Compounds were randomly selected from Table
1-Table 6, together with other compounds having only one or two cysteine-
binding potential and experimentally tested their ability in folding p53-
R175H and transcriptionally activating p53-R175H on PUMA promoter using
the PAb1620 IP assay and luciferase reporter assay, respectively. Increasing
‘+’ represents increasing transcriptional activity of p53-R175H
on PUMA promoter upon compound treatment.

| | NSC No. | Degree of mp53 | Degree of mp53 |
|---|---|---|---|
| | or Sigma | Structural | transcriptional |
| Formula | Cat No. | Rescue | activity rescue |
| KAsO₂ | NSC3060 | + | +++++ |
| AsCl₃ | Sigma 200077 | + | +++++ |
| KAsNa₂O₄ | Sigma A6756 | + | +++++ |
| NaAsO₂ | Sigma S7400 | + | +++++ |
| AsI₃ | Sigma 401145 | + | +++++ |
| As₂O₃ | Sigma 202673 | + | +++++ |
| As₂O₅ | Sigma 483257 | + | +++++ |
| KAsF₆ | Sigma 342246 | + | +++ |
| LiAsF₆ | Sigma 308315 | + | +++ |
| As₄S₄ | Sigma 448060 | + | ++++ |
| SbCl₃ | Sigma 337374 | + | +++ |
| SbF₃ | Sigma 381292 | + | ++ |
| SbAc₃ | Sigma 483265 | + | ++ |
| Sb₂O₃ | Sigma 202649 | + | +++ |
| Sb(OC₂H₅)₃ | Sigma 213314 | + | +++ |
| Sb(OCH₃)₃ | Sigma 538345 | + | +++ |
| SbI₃ | Sigma 401188 | + | +++ |
| Sb₂O₅ | Sigma 255998 | + | +++ |
| Sb₂(SO₄)₃ | Sigma 10783 | + | +++ |
| BiI₃ | Sigma 229474 | + | +++ |
| C₁₆H₁₈As₂N₄O₂ | NSC92909 | + | + |
| C₁₃H₁₄As₂O₆ | NSC48300 | + | +++ |
| C₁₇H₂₈AsClN₄O₆S | NSC721951 | + | + |
| C₁₀H₁₃NO₈Sb | NSC31660 | + | +++ |
| C₆H₁₂NaO₈Sb+ | NSC15609 | + | +++ |
| (CH₃CO₂)₃Sb | Sigma 483265 | + | + |
| C₈H₄K₂O₁₂Sb₂•xH₂O | Sigma 244791 | + | ++ |
| C₁₃H₂₁NaO₉Sb+ | NSC15623 | + | +++ |
| HOC₆H₄COOBiO | Sigma 480789 | + | + |
| [O₂CCH₂C(OH)(CO₂)CH₂CO₂]Bi | Sigma 480746 | + | +++ |
| (CH₃CO₂)₃Bi | Sigma 401587 | + | +++ |

TABLE 8
Exemplar p53 SNP

| | | | | | |
|---|---|---|---|---|---|
| P47S | P72R | V217M | G360A | R110L/P | R267W |
| P278A | R290H | N311S | E339K | S366A | |

### 6.27 Exemplar Wildtype Human p53s

Wildtype human p53 isoform a (NCBI Reference Sequence: NP_000537.3 cellular tumor antigen p53 isoform a [Homo sapiens]; NCBI Reference Sequence: NP_001119584.1, NP_001119584.1 cellular tumor antigen p53 isoform a [Homo sapiens]), also known as p53 isoform 1 (UniProt database identifier: P04637-1, sp|P04637|P53_HUMAN Cellular tumor antigen p53 OS≡Homo sapiens GN=TP53 PE=1 SV=4), also known as p53, full-length p53, and p53α. PANDA Cysteines are underlined.

393aa
MEEPQSDPSVEPPLSQETFSDLWKLLPENNVLSPLPSQAMDDLMLSPD
DIEQWETEDPGPDEAPRMPEAAPPVAPAPAAPTPAAPAPAPSWPLSSS
VPSQKTYQGSYGFRLGFLHSGTAKSVTCTYSPALNKMFCQLAKTCPVQ
LWVDSTPPPGTRVRAMAIYKQSQHMTEVVRRCPHHERCSDSDGLAPPQ
HLIRVEGNLRVEYLDDRNTFRHSVVVPYEPPEVGSDCTTIHYNYMCNS
SCMGGMNRRPILTIITLEDSSGNLLGRNSFEVRVCACPGRDRRTEEEN
LRKKGEPHHELPPGSTKRALPNNTSSSPQPKKKPLDGEYFTLQIRGRE
RFEMFPELNEALELKDAQAGKEPGGSRAHSSHLKSKKGQSTSRHKKLM
FKTEGPDSD

Wildtype human p53 isoform b (NCBI Reference Sequence: NP_001119586.1, NP_001119586.1 cellular tumor antigen p53 isoform b [Homo sapiens]), also known as p53 isoform 2 (UniProt database identifier: P04637-2, sp|P04637-2|P53_HUMAN Isoform 2 of Cellular tumor antigen p53 OS=Homo sapiens GN=TP53), also known as p53β. PANDA Cysteines are underlined.

341aa
MEEPQSDPSVEPPLSQETFSDLWKLLPENNVLSPLPSQAMDDLMLSPD
DIEQWFTEDPGPDEAPRMPEAAPPVAPAPPAPTPAAPAPAPSWPLSSS
VPSQKTYQGSYGFRLGFLHSGTAKSVTCTYSPALNKMFCQLAKTCPVQ
LWVDSTPPPGTRVRAMAIYKQSQHMTEVVRRCPHHERCSDSDGLAPPQ
HLIRVEGNLRVEYLDDRNTFRHSVVVPYEPPEVGSDCTTIHYNYMCNS
SCMGGMNRRPILTIITLEDSSGNLLGRNSFEVRVCACPGRDRRTEEEN
LRKKGEPHHELPPGSTKRALPNNTSSSPQPKKKPLDGEYFTLQDQTSF
QKENC

Wildtype human p53 isoform c (NCBI Reference Sequence: NP_001119585.1, NP_001119585.1 cellular tumor antigen p53 isoform c [Homo sapiens]) also known as p53 isoform 3 (UniProt database identifier P04637-3, sp|P04637-3|P53_HUMAN Isoform 3 of Cellular tumor antigen p53 OS=Homo sapiens GN=TP53), also known as p53γ. PANDA Cysteines are underlined.

346aa
MEEPQSDPSVEPPLSQETPSDLWKLLPENNVLSPLPSQAMDDLMLSPD
DIEQWFTEDPGPDEAPRMPEAAPPVAPAPAAPTPAAPAPAPSWPLSSS
VPSQKTYQGSYGFRLGFLHSGTAKSVTCTYSPALNKMFCQLAKTCPVQ
LWVDSTPPPGTRVRAMAIYKQSQHMTEVVRRCPHHERCSDSDGLAPPQ
HLIRVEGNLRVEYLDDRNTFRHSVVVPYEPPEVGSDCTTIHYNYMCNS
SCMGGMNRRPILTIITLEDSSGNLLGRNSFEVRVCACPGRDRRTEEEN
LRKKGEPHHELPPGSTKRALPNNTSSSPQPKKKPLDGEYFTLQMLLDL
RWCYFLINSS

Wildtype human p53 isoform g (NCBI Reference Sequence: NP_001119590.1, NP_001119590.1 cellular tumor antigen p53 isoform g [Homo sapiens]; NCBI Reference Sequence: NP_001263689.1, NP_001263689.1 cellular tumor antigen p53 isoform g [Homo sapiens]; NCBI Reference Sequence: NP_001263690.1, NP_001263690.1 cellular tumor antigen p53 isoform g [Homo sapiens]) also known as p53 isoform 4 (UniProt database identifier: P04637-4, sp|P04637-4|P53_HUMAN Isoform 4 of Cellular tumor antigen p53 OS=Homo sapiens GN=TP53), also known as A40p53α. PANDA Cysteines are underlined.

354aa
MDDLMLSPDDIEQWFTEDPGPDEAPRMPEAAPPVAPAPAAPTPAAPAP
APSWPLSSSVPSQKTYQGSYGFRLGFLHSGTAKSVTCTYSPALNKMFC
QLAKTCPVQLWVDSTPPPGTRVRAMAIYKQSQHMTEVVRRCPHHERCS
DSDGLAPPQHLIRVEGNLRVEYLDDRNTFRHSVVVPYEPPEVGSDCTT
IHYNYMCNSSCMGGMNRRPILTIITLEDSSGNLLGRNSFEVRVCACPG
RDRRTEEENLRKKGEPHHELPPGSTKRALPNNTSSSPQPKKKPLDGEY
FTLQIRGRERFEMFRELNEALELKDAQAGKEPGGSRAHSSHLKSKKGQ
STSRHKKLMFKTEGPDSD

Wildtype human p53 isoform i (NCBI Reference Sequence: NP_001263625.1, NP_001263625.1 cellular tumor antigen p53 isoform i [Homo sapiens]), also known as p53 isoform 5 (UniProt database identifier P04637-5, sp|P04637-5|P53_HUMAN Isoform 5 of Cellular tumor antigen p53 OS=Homo sapiens GN=TP53), also known as A40p53β. PANDA Cysteines are underlined.

302aa
MDDLMLSPDDIEQWFTEDPGPDEAPRMPEAAPPVAPAPAAPTPAAPAP
APSWPLSSSVPSQKTYQGSYGFRLGFLHSGTAKSVTCTYSPALNKMFC
QLAKTCPVQLWVDSTPPPGTRVRAMAIYKQSQHMTEVVRRCPHHERCS
DSDGLAPPQHLIRVEGNLRVEYLDDRNTFRNSVVVPYEPPEVGSDCTT
IHYNYNCNSSCMGGMNRRPILTIITLEDSSGYLLGRNSFEVRVCACPG
RDRRTEEENLRKKGEPHHELPPGSTKRALPNNTSSSPQPKKKPLDGEY
FTLQDQTSFQKENC

Wildtype human p53 isoform h (NCBI Reference Sequence: NP_001263624.1, NP_001263624.1 cellular tumor antigen p53 isoform h [Homo sapiens]), also known as p53 isoform 6 (UniProt database identifier: P04637-6, sp|P04637-6|P53_HUMAN Isoform 6 of Cellular tumor antigen p53 OS=Homo sapiens GN=TP53), also known as A40p53γ. PANDA Cysteines are underlined.

307aa
MDDLMLSPDDIEQWFTEDPGPDEAPRMPEAAPPVAPAPAAPTPAAPAP
APSWPLSSSVPSQKTYQGSYGFRLGFLHSGTAKSVTCTYSPALNKMFC
QLAKTCPVQLWVDSTPPPGTRVRAMAIYKQSQHMTEVVRRCPHHERCS
DSDGLAPPQHLIRVEGNLRVEYLDDRNTFRHSVVVPYEPPEVGSDCTT
IHYNYMCNSSCMGGMNRRPILTIITLEDSSGNLLGRNSFEVRVCACPG
RDRRTEEENLRKKGEPHHELPPGSTKRALPNNTSSSPQPKKKPLDGEY
FTLQMLLDLRWCYFLINSS

### 7. REFERENCES

The following publications, references, patents and patent applications are hereby incorporated by reference in their entireties.
Alexandrova, E. M., Yallowitz, A. R., Li, D., Xu, S., Schulz, R., Proia, D. A., Lozano, G., Dobbelstein, M., and Moll, U. M. (2015). Improving survival by exploiting tumour dependence on stabilized mutant p53 for treatment. Nature 523, 352-356.
Aryee, D. N., Niedan, S., Ban, J., Schwentner, R., Muehlbacher, K., Kauer, M., Kofler, R., and Kovar, H. (2013). Variability in functional p53 reactivation by PRIMA-1(Met)/APR-246 in Ewing sarcoma. British journal of cancer 109, 2696-2704.
Basse, N., Kaar, J. L., Settanni, G., Joerger, A. C., Rutherford, T. J., and Fersht, A. R. (2010). Toward the rational design of p53-stabilizing drugs: probing the surface of the oncogenic Y220C mutant. Chemistry & biology 17, 46-56.
Bauer, M. R., Joerger, A. C., and Fersht, A. R. (2016). 2-Sulfonylpyrimidines: Mild alkylating agents with anticancer activity toward p53-compromised cells. Proceedings of the National Academy of Sciences of the United States of America 113, E5271-5280.
Boeckler, F. M., Joerger, A. C., Jaggi, G., Rutherford, T. J., Veprintsev, D. B., and Fersht, A. R. (2008). Targeted rescue of a destabilized mutant of p53 by an in silico screened drug. Proceedings of the National Academy of Sciences of the United States of America 105, 10360-10365.
Bullock, A. N, and Fersht, A. R. (2001). Rescuing the function of mutant p53. Nature reviews Cancer 1, 68-76.
Bullock, A. N, Henckel, J., DeDecker, B. S., Johnson, C. M., Nikolova, P. V., Proctor, M. R., Lane, D. P., and Fersht, A. R. (1997). Thermodynamic stability of wild-type and mutant p53 core domain. Proceedings of the National Academy of Sciences of the United States of America 94, 14338-14342.
Bullock, A. N, Henckel, J., and Fersht, A. R. (2000). Quantitative analysis of residual folding and DNA binding in mutant p53 core domain: definition of mutant states for rescue in cancer therapy. Oncogene 19, 1245-1256.
Bykov, V. J., Issaeva, N., Shilov, A., Hultcrantz, M., Pugacheva, E., Chumakov, P., Bergman, J., Wiman, K. G., and Selivanova, G. (2002). Restoration of the tumor suppressor function to mutant p53 by a low-molecular-weight compound. Nature medicine 8, 282-288.
Cancer Genome Atlas Research, N., Ley, T. J., Miller, C., Ding, L., Raphael, B. J., Mungall, A. J., Robertson, A., Hoadley, K., Triche, T. J., Jr., Laird, P. W., et al. (2013). Genomic and epigenomic landscapes of adult de novo acute myeloid leukemia. The New England journal of medicine 368, 2059-2074.
Demma, M., Maxwell, E., Ramos, R., Liang, L., Li, C., Hesk, D., Rossman, R., Mallams, A., Doll, R., Liu, M., et al. (2010). SCH529074, a small molecule activator of mutant p53, which binds p53 DNA binding domain (DBD), restores growth-suppressive function to mutant p53 and interrupts HDM2-mediated ubiquitination of wild type p53. The Journal of biological chemistry 285, 10198-10212.
Demma, M. J., Wong, S., Maxwell, E., and Dasmahapatra, B. (2004). CP-31398 restores DNA-binding activity to mutant p53 in vitro but does not affect p53 homologs p63 and p73. The Journal of biological chemistry 279, 45887-45896.
Dolgin, E. (2017). The most popular genes in the human genome. Nature 551, 427-431.
Donoghue, N., Yam, P. T., Jiang, X. M., and Hogg, P. J. (2000). Presence of closely spaced protein thiols on the surface of mammalian cells. Protein science: a publication of the Protein Society 9, 2436-2445.
Feldser, D. M., Kostova, K. K., Winslow, M. M., Taylor, S. E., Cashman, C., Whittaker, C. A., Sanchez-Rivera, F. J., Resnick, R., Bronson, R., Hemann, M. T., et al. (2010). Stage-specific sensitivity to p53 restoration during lung cancer progression. Nature 468, 572-575.
Fogal, V., Hsieh, J. K., Royer, C., Zhong, S., and Lu, X. (2005). Cell cycle-dependent nudear retention of p53 by E2F1 requires phosphorylation of p53 at Ser315. The EMBO journal 24, 2768-2782.
Foster, B. A., Coffey, H. A., Morin, M. J., and Rastinejad, F. (1999). Pharmacological rescue of mutant p53 conformation and function. Science 286, 2507-2510.
Freed-Pastor, W. A., and Prives, C. (2012). Mutant p53: one name, many proteins. Genes & development 26, 1268-1286.
Gao, J., Aksoy, B. A., Dogrusoz, U., Dresdner, G., Gross, B., Sumer, S. O., Sun, Y., Jacobsen, A., Sinha, R., Larsson, E., et al. Integrative analysis of complex cancer genomics and clinical profiles using the cBioPortal. Sci Signal 6, p11.
Gillotin, S., Yap, D., and Lu, X. (2010). Mutation at Ser392 specifically sensitizes mutant p53H₁₇₅ to mdm2-mediated degradation. Cell cycle 9, 1390-1398.
Grellety, T., Laroche-Clary, A., Chaire, V., Lagarde, P., Chibon, F., Neuville, A., and Italiano, A. (2015). PRIMA-1(MET) induces death in soft-tissue sarcomas cell independent of p53. BMC cancer 15, 684.
Heredia-Moya, J., and Kirk, K. L. (2008). An improved synthesis of arsenic-biotin conjugates. Bioorganic & medicinal chemistry 16, 5743-5746.
Hu, J., Liu, Y. F., Wu, C. F., Xu, F., Shen, Z. X., Zhu, Y. M., Li, J. M., Tang, W., Zhao, W. L., Wu, W., et al. (2009). Long-term efficacy and safety of all-trans retinoic acid/arsenic trioxide-based therapy in newly diagnosed acute promyelocytic leukemia. Proceedings of the National Academy of Sciences of the United States of America 106, 3342-3347.
Joerger, A. C., and Fersht, A. R. (2007). Structure-function-rescue: the diverse nature of common p53 cancer mutants. Oncogene 26, 2226-2242.
Joerger, A. C., and Fersht, A. R. (2016). The p53 Pathway: Origins, Inactivation in Cancer, and Emerging Therapeutic Approaches. Annual review of biochemistry 85, 375-404.
Kandoth, C., McLellan, M. D., Vandin, F., Ye, K., Niu, B., Lu, C., Xie, M., Zhang, Q., McMichael, J. F., Wyczalkowski, M. A., et al. (2013). Mutational landscape and significance across 12 major cancer types. Nature 502, 333-339.
Khoo, K. H., Verma, C. S., and Lane, D. P. (2014). Drugging the p53 pathway: understanding the route to clinical efficacy. Nature reviews Drug discovery 13, 217-236.
Lambert, J. M., Gorzov, P., Veprintsev, D. B., Soderqvist, M., Segerback, D., Bergman, J., Fersht, A. R., Hainaut, P., Wiman, K. G., and Bykov, V. J. (2009). PRIMA-1 reactivates mutant p53 by covalent binding to the core domain. Cancer cell 15, 376-388.
Li, D., Marchenko, N. D., and Moll, U. M. (2011). SAHA shows preferential cytotoxicity in mutant p53 cancer cells by destabilizing mutant p53 through inhibition of the HDAC6-Hsp90 chaperone axis. Cell death and differentiation 18, 1904-1913.
Lindsley, R. C., Saber, W., Mar, B. G., Redd, R., Wang, T., Haagenson, M. D., Grauman, P. V., Hu, Z. H., Spellman, S. R., Lee, S. J., et al. (2017). Prognostic Mutations in Myelodysplastic Syndrome after Stem-Cell Transplantation. The New England journal of medicine 376, 536-547.
Liu, X., Wilcken, R., Joerger, A. C., Chuckowree, I. S., Amin, J., Spencer, J., and Fersht, A. R. (2013). Small molecule induced reactivation of mutant p53 in cancer cells. Nucleic acids research 41, 6034-6044.
Lo-Coco, F., Avvisati, G., Vignetti, M., Thiede, C., Orlando, S. M., Iacobelli, S., Ferrara, F., Fazi, P., Cicconi, L., Di Bona, E., et al. (2013). Retinoic acid and arsenic trioxide for acute promyelocytic leukemia. The New England journal of medicine 369, 111-121.
Lu, M., Breyssens, H., Salter, V., Zhong, S., Hu, Y., Baer, C., Ratnayaka, I., Sullivan, A., Brown, N. R., Endicott, J., et al. (2013). Restoring p53 function in human melanoma cells by inhibiting MDM2 and cyclin B1/CDK1-phosphorylated nuclear iASPP. Cancer cell 23, 618-633.
Lu, M., Muers, M. R., and Lu, X. (2016a). Introducing STRaNDs: shuttling transcriptional regulators that are non-DNA binding. Nature reviews Molecular cell biology 17, 523-532.
Lu, M., Breyssens, H., Salter, V., Zhong, S., Hu, Y., Baer, C., Ratnayaka, I., Sullivan, A., Brown, N. R., Endicott, J., et al. (2013). Restoring p53 function in human melanoma cells by inhibiting MDM2 and cyclin B1/CDK1-phosphorylated nuclear iASPP. Cancer cell 23, 618-633.
Lu, M., Zak, J., Chen, S., Sanchez-Pulido, L., Severson, D. T., Endicott, J., Ponting, C. P., Schofield, C. J., and Lu, X. (2014). A code for RanGDP binding in ankyrin repeats defines a nuclear import pathway. Cell 157, 1130-1145.
Lu, T., Zou, Y., Xu, G., Potter, J. A., Taylor, G. L., Duan, Q., Yang, Q., Xiong, H., Qiu, H., Ye, D., et al. (2016b). PRIMA-1Met suppresses colorectal cancer independent of p53 by targeting MEK. Oncotarget.
Lukashchuk, N., and Vousden, K. H. (2007). Ubiquitination and degradation of mutant p53. Molecular and cellular biology 27, 8284-8295.
Martins, C. P., Brown-Swigart, L., and Evan, G. I. (2006). Modeling the therapeutic efficacy of p53 restoration in tumors. Cell 127, 1323-1334.
Muller, P. A., Caswell, P. T., Doyle, B., Iwanicki, M. P., Tan, E. H., Karim, S., Lukashchuk, N., Gillespie, D. A., Ludwig, R. L., Gosselin, P., et al. (2009). Mutant p53 drives invasion by promoting integrin recycling. Cell 139, 1327-1341.
Muller, P. A., and Vousden, K. H. (2013). p53 mutations in cancer. Nature cell biology 15, 2-8.
Muller, P. A., and Vousden, K. H. (2014). Mutant p53 in cancer new functions and therapeutic opportunities. Cancer cell 25, 304-317.
Parrales, A., Ranjan, A., lyer, S. V., Padhye, S., Weir, S. J., Roy, A., and Iwakuma, T. (2016). DNAJA1 controls the fate of misfolded mutant p53 through the mevalonate pathway. Nature cell biology 18, 1233-1243.
Patyka, M., Sharifi, Z., Petrecca, K., Mansure, J., Jean-Claude, B., and Sabri, S. (2016). Sensitivity to PRIMA-1MET is associated with decreased MGMT in human glioblastoma cells and glioblastoma stem cells irrespective of p53 status. Oncotarget.
Puca, R., Nardinocchi, L., Porru, M., Simon, A. J., Rechavi, G., Leonetti, C., Givol, D., and D'Orazi, G. (2011). Restoring p53 active conformation by zinc increases the response of mutant p53 tumor cells to anticancer drugs. Cell cycle 10, 1679-1689.
Riley, T., Sontag, E., Chen, P., and Levine, A. (2008). Transcriptional control of human p53-regulated genes. Nature reviews Molecular cell biology 9, 402-412.
Rippin, T. M., Bykov, V. J., Freund, S. M., Selivanova, G., Wiman, K. G., and Fersht, A. R. (2002). Characterization of the p53-rescue drug CP-31398 in vitro and in living cells. Oncogene 21, 2119-2129.
Shoemaker, R. H. (2006). The NCI60 human tumour cell line anticancer drug screen. Nature reviews Cancer 6, 813-823.
Soragni, A., Janzen, D. M., Johnson, L. M., Lindgren, A. G., Thai-Quynh Nguyen, A., Tiourin, E., Soriaga, A. B., Lu, J., Jiang, L., Faull, K. F., et al. (2016). A Designed Inhibitor of p53 Aggregation Rescues p53 Tumor Suppression in Ovarian Carcinomas. Cancer cell 29, 90-103.
Tessoulin, B., Descamps, G., Moreau, P., Maiga, S., Lode, L., Godon, C., Marionneau-Lambot, S., Oullier, T., Le Gouill, S., Amiot, M., et al. (2014). PRIMA-1Met induces myeloma cell death independent of p53 by impairing the GSH/ROS balance. Blood 124, 1626-1636.
Vassilev, L. T., Vu, B. T., Graves, B., Carvajal, D., Podlaski, F., Filipovic, Z., Kong, N., Kammlott, U., Lukacs, C., Klein, C., et al. (2004). In vivo activation of the p53 pathway by small-molecule antagonists of MDM2. Science 303, 844-848.
Ventura, A., Kirsch, D. G., McLaughlin, M. E., Tuveson, D. A., Grimm, J., Lintault, L., Newman, J., Reczek, E. E., Weissleder, R., and Jacks, T. (2007). Restoration of p53 function leads to tumour regression in vivo. Nature 445, 661-665.
Vogelstein, B., Lane, D., and Levine, A. J. (2000). Surfing the p53 network. Nature 408, 307-310.
Wang, Y., Suh, Y. A., Fuller, M. Y., Jackson, J. G., Xiong, S., Terzian, T., Quintas-Cardama, A., Bankson, J. A., El-Naggar, A. K., and Lozano, G. (2011). Restoring expression of wild-type p53 suppresses tumor growth but does not cause tumor regression in mice with a p53 missense mutation. The Journal of clinical investigation 121, 893-904.
Wassman, C. D., Baronio, R., Demir, O., Wallentine, B. D., Chen, C. K., Hall, L. V., Salehi, F., Lin, D. W., Chung, B. P., Hatfield, G. W., et al. (2013). Computational identification of a transiently open L1/S3 pocket for reactivation of mutant p53. Nature communications 4, 1407.
Weinmann, L., Wischhusen, J., Demma, M. J., Naumann, U., Roth, P., Dasmahapatra, B., and Weller, M. (2008). A novel p53 rescue compound induces p53-dependent growth arrest and sensitises glioma cells to Apo2L/TRAIL-induced apoptosis. Cell death and differentiation 15, 718-729.
Xue, W., Zender, L., Miething, C., Dickins, R. A., Hemando, E., Krizhanovsky, V., Cordon-Cardo, C., and Lowe, S. W. (2007). Senescence and tumour clearance is triggered by p53 restoration in murine liver carcinomas. Nature 445, 656-660.
Zhang, T. D., Chen, G. Q., Wang, Z. G., Wang, Z. Y., Chen, S. J., and Chen, Z. (2001). Arsenic trioxide, a therapeutic agent for APL. Oncogene 20, 7146-7153.
Zhang, X. W., Yan, X. J., Zhou, Z. R., Yang, F. F., Wu, Z. Y., Sun, H. B., Liang, W. X., Song, A. X., Lallemand-Breitenbach, V., Jeanne, M., et al. (2010). Arsenic troxide controls the fate of the PML-RARalpha oncoprotein by directly binding PML. Science 328, 240-243.
Zhu, J., Chen, Z., Lallemand-Breitenbach, V., and de The, H. (2002). How acute promyelocytic leukaemia revived arsenic. Nature reviews Cancer 2, 705-713.

APPENDIX A
Atomic Coordinates of FIG. 14 Left Panel (in vivo formed PANDA)

| HEADER | — | | | | 14 SEP. 17 | xxxx | | | | | | |
|---|---|---|---|---|---|---|---|---|---|---|---|---|
| COMPND | — | | | | | | | | | | | |
| REMARK | 3 | REFINEMENT. | | | | | | | | | | |
| REMARK | 3 | PROGRAM: | | REFMAC | | | | | | | | |
| | | | | 5.8.01.58 | | | | | | | | |
| REMARK | 3 | AUTHORS: | | MURSHUDOV, | | | | | | | | |
| REMARK | 3 | | | SKUBAK, | | | | | | | | |
| REMARK | 3 | | | LEBEDEV, | | | | | | | | |
| | | | | PANNU, | | | | | | | | |
| | | | | STEINER, | | | | | | | | |
| | | | | NICHOLLS, | | | | | | | | |
| | | | | WINN, LONG, | | | | | | | | |
| | | | | VAGIN | | | | | | | | |
| REMARK | 3 | REFINEMENT TARGET: | | | | | | | | | | |
| | | MAXIMUM LIKELIHOOD | | | | | | | | | | |
| REMARK | 3 | | | | | | | | | | | |
| REMARK | 3 | DATA USED IN REFINEMENT. | | | | | | | | | | |
| REMARK | 3 | RESOLUTION | | (ANGSTROMS): | 1.92 | | | | | | | |
| | | RANGE HIGH | | | | | | | | | | |
| REMARK | 3 | RESOLUTION | | (ANGSTROMS): | 66.55 | | | | | | | |
| | | RANGE LOW | | | | | | | | | | |
| REMARK | 3 | DATA CUTOFF | | (SIGMA (F)): | NONE | | | | | | | |
| REMARK | 3 | COMPLETENESS | | (%): | 94.96 | | | | | | | |
| | | FOR RANGE | | | | | | | | | | |
| REMARK | 3 | NUMBER OF | | | 26004 | | | | | | | |
| | | REFLECTIONS: | | | | | | | | | | |
| REMARK | 3 | | | | | | | | | | | |
| REMARK | 3 | FIT TO DATA USED IN REFINEMENT. | | | | | | | | | | |
| REMARK | 3 | CROSS-VALIDATION METHOD: | | THROUGHOUT | | | | | | | | |
| REMARK | 3 | FREE R VALUE TEST SET SELECTION: | | RANDOM | | | | | | | | |
| REMARK | 3 | R VALUE | | (WORKING + | 0.28464 | | | | | | | |
| | | | | TEST SET): | | | | | | | | |
| REMARK | 3 | R VALUE | | (WORKING SET): | 0.28210 | | | | | | | |
| REMARK | 3 | FREE R VALUE: | | | 0.33681 | | | | | | | |
| REMARK | 3 | FREE R VALUE | | (%): | 4.5 | | | | | | | |
| | | TEST SET SIZE | | | | | | | | | | |
| REMARK | 3 | FREE R VALUE | | | 1224 | | | | | | | |
| | | TEST SET COUNT: | | | | | | | | | | |
| REMARK | 3 | | | | | | | | | | | |
| REMARK | 3 | FIT IN THE HIGHEST | | | | | | | | | | |
| | | RESOLUTION BIN. | | | | | | | | | | |
| REMARK | 3 | TOTAL NUMBER | | | 20 | | | | | | | |
| | | OF BINS USED: | | | | | | | | | | |
| REMARK | 3 | BIN RESOLUTION | | | 1.920 | | | | | | | |
| | | RANGE HIGH: | | | | | | | | | | |
| REMARK | 3 | BIN RESOLUTION | | | 1.970 | | | | | | | |
| | | RANGE LOW: | | | | | | | | | | |
| REMARK | 3 | REFLECTION | | (WORKING SET): | 1903 | | | | | | | |
| | | IN BIN | | | | | | | | | | |
| REMARK | 3 | BIN | | (WORKING + | 94.30 | | | | | | | |
| | | COMPLETENESS | | TEST) (%): | | | | | | | | |
| REMARK | 3 | BIN R VALUE | | (WORKING SET): | 0.371 | | | | | | | |
| REMARK | 3 | BIN FREE R VALUE | | | 99 | | | | | | | |
| | | SET COUNT: | | | | | | | | | | |
| REMARK | 3 | BIN FREE P VALUE: | | | 0.417 | | | | | | | |
| REMARK | 3 | | | | | | | | | | | |
| REMARK | 3 | NUMBER OF | | | | | | | | | | |
| | | NON-HYDROGEN | | | | | | | | | | |
| | | ATOMS USED IN | | | | | | | | | | |
| | | REFINEMENT. | | | | | | | | | | |
| REMARK | 3 | ALL ATOMS: | | 3196 | | | | | | | | |
| REMARK | 3 | | | | | | | | | | | |
| REMARK | 3 | B VALUES | | | | | | | | | | |
| REMARK | 3 | FROM WILSON PLOT | | (A**2): | NULL | | | | | | | |
| REMARK | 3 | MEAN L VALUE | | (OVERALL, A**2): | 24.184 | | | | | | | |
| REMARK | 3 | OVERALL ANISOTROPIC B VALUE. | | | | | | | | | | |
| REMARK | 3 | B11 | | (A**2): | 0.23 | | | | | | | |
| REMARK | 3 | B22 | | (A**2): | −1.72 | | | | | | | |
| REMARK | 3 | B33 | | (A**2): | 1.28 | | | | | | | |
| REMARK | 3 | B12 | | (A**2): | 0.00 | | | | | | | |
| REMARK | 3 | B13 | | (A**2): | 1.69 | | | | | | | |
| REMARK | 3 | B23 | | (A**2): | 0.00 | | | | | | | |
| REMARK | 3 | | | | | | | | | | | |
| REMARK | 3 | ESTIMATED | | | | | | | | | | |
| | | OVERALL | | | | | | | | | | |
| | | COORDINATE | | | | | | | | | | |
| | | ERROR. | | | | | | | | | | |
| REMARK | 3 | ESU- BASED ON | | (A): | 0.275 | | | | | | | |
| | | R VALUE | | | | | | | | | | |
| REMARK | 3 | ESU BASED ON | | (A): | 0.234 | | | | | | | |
| | | FREE R VALUE | | | | | | | | | | |
| REMARK | 3 | ESU BASED ON | | (A): | 0.258 | | | | | | | |
| | | MAXIMUM | | | | | | | | | | |
| | | LIKELIHOOD | | | | | | | | | | |
| REMARK | 3 | ESU FOR B VALUES | | (A**2): | 9.225 | | | | | | | |
| | | BASED ON | | | | | | | | | | |
| | | MAXIMUM | | | | | | | | | | |
| | | LIKELIHOOD | | | | | | | | | | |
| REMARK | 3 | | | | | | | | | | | |
| REMARK | 3 | CORRELATION | | | | | | | | | | |
| | | COEFFICIENTS. | | | | | | | | | | |
| REMARK | 3 | CORRELATION | | | 0.877 | | | | | | | |
| | | COEFFICIENT FO-FC: | | | | | | | | | | |
| REMARK | 3 | CORRELATION | | | 0.823 | | | | | | | |
| | | COEFFICIENT | | | | | | | | | | |
| | | FO-FC FREE: | | | | | | | | | | |
| REMARK | 3 | | | | | | | | | | | |
| REMARK | 3 | RMS | | | COUNT | RMS | WEIGHT | | | | | |
| | | DEVIATIONS | | | | | | | | | | |
| | | FROM IDEAL | | | | | | | | | | |
| | | VALUES | | | | | | | | | | |
| REMARK | 3 | BOND LENGTHS | | (A): | 3109; | 0.007; | 0.019 | | | | | |
| | | REFINED ATOMS | | | | | | | | | | |
| REMARK | 3 | BOND LENGTHS | | (A): | 2785; | 0.002; | 0.020 | | | | | |
| | | OTHERS | | | | | | | | | | |
| REMARK | 3 | BOND ANGLES | | (DEGREES): | 4210; | 1.173; | 1.960 | | | | | |
| | | REFINED ATOMS | | | | | | | | | | |
| REMARK | 3 | BOND ANGLES | | (DEGREES): | 6467; | 0.906; | 3.004 | | | | | |
| | | OTHERS | | | | | | | | | | |
| REMARK | 3 | TORSION ANGLES, | | (DEGREES): | 384; | 6.559; | 5.000 | | | | | |
| | | PERIOD 1 | | | | | | | | | | |
| REMARK | 3 | TORSION ANGLES, | | (DEGREES): | 145; | 31.368; | 22.690 | | | | | |
| | | PERIOD 2 | | | | | | | | | | |
| REMARK | 3 | TORSION ANGLES, | | (DEGREES): | 516; | 13.571; | 15.000 | | | | | |
| | | PERIOD 3 | | | | | | | | | | |
| REMARK | 3 | TORSION ANGLES, | | (DEGREES): | 32; | 11.949; | 15.000 | | | | | |
| | | PERIOD 4 | | | | | | | | | | |
| REMARK | 3 | CHIRAL-CENTER | | (A**3): | 459; | 0.066; | 0.200 | | | | | |
| | | RESTRAINTS | | | | | | | | | | |
| REMARK | 3 | GENERAL PLANES | | (A): | 3461; | 0.005; | 0.021 | | | | | |
| | | REFINED ATOMS | | | | | | | | | | |
| REMARK | 3 | GENERAL PLANES | | (A): | 639; | 0.001; | 0.020 | | | | | |
| | | OTHERS | | | | | | | | | | |
| REMARK | 3 | | | | | | | | | | | |
| REMARK | 3 | ISOTROPIC | | | COUNT | RMS | WEIGHT | | | | | |
| | | THERMAL | | | | | | | | | | |
| | | FACTOR | | | | | | | | | | |
| | | RESTRAINTS. | | | | | | | | | | |
| REMARK | 3 | MAIN-CHAIN BOND | | (A**2): | 1539; | 0.721; | 2.497 | | | | | |
| | | REFINED ATOMS | | | | | | | | | | |
| REMARK | 3 | MAIN-CHAIN BOND | | (A**2): | 1538; | 0.721; | 2.496 | | | | | |
| | | OTHER ATOMS | | | | | | | | | | |
| REMARK | 3 | MAIN-CHAIN ANGLE | | (A**2): | 1922; | 1.331; | 3.739 | | | | | |
| | | REFINED ATOMS | | | | | | | | | | |
| REMARK | 3 | MAIN-CHAIN ANGLE | | (A**2): | 1923; | 1.330; | 3.740 | | | | | |
| | | OTHER ATOMS | | | | | | | | | | |
| REMARK | 3 | SIDE-CHAIN BOND | | (A**2): | 1570; | 0.670; | 2.492 | | | | | |
| | | REFINED ATOMS | | | | | | | | | | |
| REMARK | 3 | SIDE-CHAIN BOND | | (A**2): | 1571; | 0.670; | 2.493 | | | | | |
| | | OTHER ATOMS | | | | | | | | | | |
| REMARK | 3 | SIDE-CHAIN ANGLE | | (A**2): | 2289; | 0.813; | 3.714 | | | | | |
| | | OTHER ATOMS | | | | | | | | | | |
| REMARK | 3 | LONG RANGE B | | (A**2): | 3347; | 3.790; | 29.140 | | | | | |
| | | REFINED ATOMS | | | | | | | | | | |
| REMARK | 3 | LONG RANGE B | | (A**2): | 3326; | 3.624; | 29.116 | | | | | |
| | | OTHER ATOMS | | | | | | | | | | |
| REMARK | 3 | | | | | | | | | | | |
| REMARK | 3 | NCS RESTRAINTS | | | | | | | | | | |
| | | STATISTICS | | | | | | | | | | |
| REMARK | 3 | NUMBER OF | | NULL | | | | | | | | |
| | | NCS GROUPS: | | | | | | | | | | |
| REMARK | 3 | | | | | | | | | | | |
| REMARK | 3 | TWIN DETAILS | | | | | | | | | | |
| REMARK | 3 | NUMBER OF TWIN | | NULL | | | | | | | | |
| | | DOMAINS: | | | | | | | | | | |
| REMARK | 3 | | | | | | | | | | | |
| REMARK | 3 | | | | | | | | | | | |
| REMARK | 3 | TLS DETAILS | | | | | | | | | | |
| REMARK | 3 | NUMBER OF | | NULL | | | | | | | | |
| | | TLS GROUPS: | | | | | | | | | | |
| REMARK | 3 | | | | | | | | | | | |
| REMARK | 3 | | | | | | | | | | | |
| REMARK | 3 | BULK SOLVENT | | | | | | | | | | |
| | | MODELLING. | | | | | | | | | | |
| REMARK | 3 | METHOD USED: | | MASK | | | | | | | | |
| REMARK | 3 | PARAMETERS | | | | | | | | | | |
| | | FOR MASK | | | | | | | | | | |
| | | CALCULATION | | | | | | | | | | |
| REMARK | 3 | VOW PROBE RADIUS: | | 1.20 | | | | | | | | |
| REMARK | 3 | ION PROBE RADIUS: | | 0.80 | | | | | | | | |
| REMARK | 3 | SHRINKAGE RADIUS: | | 0.80 | | | | | | | | |
| REMARK | 3 | | | | | | | | | | | |
| REMARK | 3 | OTHER REFINEMENT | | | | | | | | | | |
| | | REMARKS: | | | | | | | | | | |
| REMARK | 3 | HYDROGENS HAVE | | | | | | | | | | |
| | | BEEN ADDED IN THE | | | | | | | | | | |
| | | RIDING POSITIONS | | | | | | | | | | |
| REMARK | 3 | U VALUES: | | REFINED | | | | | | | | |
| | | | | INDIVIDUALLY | | | | | | | | |
| REMARK | 3 | | | | | | | | | | | |
| LINK | AS | ARS | | D | 1 | SG | CYS | A | 141 | 1555 | 1555 | 2.14 |
| LINK | AS | ARS | | D | 1 | SG | CYS | A | 124 | 1555 | 1555 | 2.14 |
| LINK | AS | ARS | | D | 1 | SG | CYS | A | 135 | 1555 | 1555 | 2.14 |
| LINK | AS | ARS | | D | 2 | SG | CYS | B | 124 | 1555 | 1555 | 2.14 |
| LINK | AS | ARS | | D | 2 | SG | CYS | B | 135 | 1555 | 1555 | 2.14 |
| LINK | AS | ARS | | D | 2 | SG | CYS | B | 141 | 1555 | 1555 | 2.14 |
| LINKR | ZN | ZN | | C | 1 | SG | CYS | A | 242 | | | ZN-CYS |
| LINKR | ZN | ZN | | C | 1 | SG | CYS | A | 176 | | | ZN-CYS |
| LINKR | ZN | ZN | | C | 1 | SG | CYS | A | 238 | | | ZN-CYS |
| LINKR | ZN | ZN | | C | 1 | ND1 | HIS | A | 179 | | | ZN- |
| | | | | | | | | | | | | HISND |
| LINER | ZN | ZN | | C | 2 | SG | CYS | B | 238 | | | ZN-CYS |
| LINER | ZN | ZN | | C | 2 | SG | CYS | B | 176 | | | ZN-CYS |
| LINER | ZN | ZN | | C | 2 | SG | CYS | B | 242 | | | ZN-CYS |
| LINER | ZN | ZN | | C | 2 | ND1 | HIS | B | 179 | | | ZN- |
| | | | | | | | | | | | | HISND |
| LINKP | | CYS | | B | 242 | | ILE | B | 251 | | | gap |
| LINKP | | MET | | A | 243 | | SER | A | 249 | | | gap |
| LINEN | | LYS | | B | 164 | | SER | B | 166 | | | gap |
| LINER | | SER | | B | 240 | | ILE | B | 251 | | | gap |
| CRYST1 | 41.510 | 68.760 | 66.670 | 90.00 | 93.48 | 90.00 | P 1 21 1 | | | | | |
| SCALE1 | | 0.024091 | 0.000000 | 0.001466 | 0.00000 | | | | | | | |
| SCALE2 | | −0.000000 | 0.014543 | 0.000000 | 0.00000 | | | | | | | |
| SCALES | | 0.000000 | −0.000000 | 0.015027 | 0.000000 | | | | 0.00000 | | | |

| | | | | | | | | | | | |
|---|---|---|---|---|---|---|---|---|---|---|---|
| ATOM | 1 | N | SER | A | 95 | −86.876 | 42.991 | 11.004 | 1.00 | 44.58 | N |
| ATOM | 2 | CA | SER | A | 95 | −86.567 | 44.261 | 11.736 | 1.00 | 44.18 | C |
| ATOM | 3 | CB | SER | A | 95 | −85.479 | 45.056 | 11.003 | 1.00 | 44.88 | C |
| ATOM | 4 | OG | SER | A | 95 | −85.878 | 45.367 | 9.680 | 1.00 | 45.67 | O |
| ATOM | 5 | C | SER | A | 95 | −87.811 | 45.132 | 11.925 | 1.00 | 43.43 | C |
| ATOM | 6 | O | SER | A | 95 | −88.871 | 44.862 | 11.353 | 1.00 | 43.65 | O |
| ATOM | 7 | N | SER | A | 96 | −87.660 | 46.173 | 12.739 | 1.00 | 41.71 | N |
| ATOM | 8 | CA | SER | A | 96 | −88.741 | 47.114 | 13.034 | 1.00 | 40.07 | C |
| ATOM | 9 | CB | SER | A | 96 | −89.744 | 46.465 | 13.994 | 1.00 | 40.26 | C |
| ATOM | 10 | OG | SER | A | 96 | −90.676 | 47.410 | 14.501 | 1.00 | 41.29 | O |
| ATOM | 11 | C | SER | A | 96 | −88.183 | 48.405 | 13.638 | 1.00 | 38.37 | C |
| ATOM | 12 | O | SER | A | 96 | −87.008 | 48.465 | 14.015 | 1.00 | 38.40 | O |
| ATOM | 13 | N | VAL | A | 97 | −89.023 | 49.437 | 13.699 | 1.00 | 35.99 | N |
| ATOM | 14 | CA | VAL | A | 97 | −88.697 | 50.679 | 14.408 | 1.00 | 34.73 | C |
| ATOM | 15 | CB | VAL | A | 97 | −88.278 | 51.819 | 13.449 | 1.00 | 34.80 | C |
| ATOM | 16 | CG1 | VAL | A | 97 | −87.762 | 53.018 | 14.241 | 1.00 | 34.96 | C |
| ATOM | 17 | CG2 | VAL | A | 97 | −87.218 | 51.347 | 12.462 | 1.00 | 34.70 | C |
| ATOM | 18 | C | VAL | A | 97 | −89.927 | 51.121 | 15.208 | 1.00 | 33.50 | C |
| ATOM | 19 | O | VAL | A | 97 | −90.929 | 51.508 | 14.606 | 1.00 | 33.90 | O |
| ATOM | 20 | N | PRO | A | 98 | −89.861 | 51.073 | 16.559 | 1.00 | 31.36 | N |
| ATOM | 21 | CA | PRO | A | 98 | −91.011 | 51.509 | 17.364 | 1.00 | 30.05 | C |
| ATOM | 22 | CB | PRO | A | 98 | −90.619 | 51.115 | 18.792 | 1.00 | 30.45 | C |
| ATOM | 23 | CG | PRO | A | 98 | −89.139 | 51.021 | 18.778 | 1.00 | 30.79 | C |
| ATOM | 24 | CD | PRO | A | 98 | −88.759 | 50.574 | 17.403 | 1.00 | 31.25 | C |
| ATOM | 25 | C | PRO | A | 98 | −91.276 | 53.013 | 17.272 | 1.00 | 28.57 | C |
| ATOM | 26 | O | PRO | A | 98 | −90.346 | 53.797 | 17.070 | 1.00 | 28.41 | O |
| ATOM | 27 | N | SER | A | 99 | −92.540 | 53.394 | 17.432 | 1.00 | 26.64 | N |
| ATOM | 28 | CA | SER | A | 99 | −92.979 | 54.778 | 17.240 | 1.00 | 25.89 | C |
| ATOM | 29 | CB | SER | A | 99 | −94.513 | 54.853 | 17.260 | 1.00 | 25.60 | C |
| ATOM | 30 | OG | SER | A | 99 | −94.971 | 56.174 | 17.040 | 1.00 | 25.95 | O |
| ATOM | 31 | C | SER | A | 99 | −92.406 | 55.718 | 18.301 | 1.00 | 24.69 | C |
| ATOM | 32 | O | SER | A | 99 | −92.205 | 55.310 | 19.442 | 1.00 | 24.14 | O |
| ATOM | 33 | N | GLN | A | 100 | −92.134 | 56.962 | 17.904 | 1.00 | 24.42 | N |
| ATOM | 34 | CA | GLN | A | 100 | −91.755 | 58.038 | 18.836 | 1.00 | 24.28 | C |
| ATOM | 35 | CB | GLN | A | 100 | −90.358 | 58.591 | 18.498 | 1.00 | 24.46 | C |
| ATOM | 36 | CG | GLN | A | 100 | −90.246 | 59.425 | 17.223 | 1.00 | 24.39 | C |
| ATOM | 37 | CD | GLN | A | 100 | −88.813 | 59.847 | 16.937 | 1.00 | 24.37 | C |
| ATOM | 38 | OE1 | GLN | A | 100 | −87.966 | 59.018 | 16.607 | 1.00 | 24.05 | O |
| ATOM | 39 | NE2 | GLN | A | 100 | −88.538 | 61.140 | 17.064 | 1.00 | 24.25 | N |
| ATOM | 40 | C | GLN | A | 100 | −92.797 | 59.162 | 18.862 | 1.00 | 23.91 | C |
| ATOM | 41 | O | GLN | A | 100 | −92.500 | 60.282 | 19.287 | 1.00 | 24.45 | O |
| ATOM | 42 | N | LYS | A | 101 | −94.019 | 58.855 | 18.425 | 1.00 | 23.59 | N |
| ATOM | 43 | CA | LYS | A | 101 | −95.100 | 59.838 | 18.376 | 1.00 | 23.26 | C |
| ATOM | 44 | CB | LYS | A | 101 | −96.251 | 59.354 | 17.481 | 1.00 | 23.95 | C |
| ATOM | 45 | CG | LYS | A | 101 | −95.967 | 59.504 | 15.997 | 1.00 | 24.31 | C |
| ATOM | 46 | CD | LYS | A | 101 | −97.157 | 59.082 | 15.151 | 1.00 | 24.54 | C |
| ATOM | 47 | CE | LYS | A | 101 | −96.808 | 59.112 | 13.673 | 1.00 | 24.66 | C |
| ATOM | 48 | NZ | LYS | A | 101 | −97.977 | 58.775 | 12.814 | 1.00 | 24.74 | N |
| ATOM | 49 | C | LYS | A | 101 | −95.623 | 60.116 | 19.775 | 1.00 | 22.76 | C |
| ATOM | 50 | O | LYS | A | 101 | −96.087 | 59.202 | 20.461 | 1.00 | 22.20 | O |
| ATOM | 51 | N | THR | A | 102 | −95.544 | 61.379 | 20.196 | 1.00 | 22.17 | N |
| ATOM | 52 | CA | THR | A | 102 | −96.134 | 61.805 | 21.460 | 1.00 | 21.82 | C |
| ATOM | 53 | CB | THR | A | 102 | −95.877 | 63.304 | 21.735 | 1.00 | 21.53 | O |
| ATOM | 54 | OG1 | THR | A | 102 | −94.468 | 63.521 | 21.879 | 1.00 | 21.23 | O |
| ATOM | 55 | CG2 | THR | A | 102 | −96.589 | 63.778 | 23.002 | 1.00 | 21.41 | C |
| ATOM | 56 | C | THR | A | 102 | −97.633 | 61.504 | 21.439 | 1.00 | 21.91 | C |
| ATOM | 57 | O | THR | A | 102 | −98.302 | 61.681 | 20.421 | 1.00 | 21.53 | O |
| ATOM | 58 | N | THR | A | 103 | −98.141 | 61.022 | 22.566 | 1.00 | 22.51 | N |
| ATOM | 59 | CA | TYR | A | 103 | −99.525 | 60.592 | 22.665 | 1.00 | 22.97 | C |
| ATOM | 60 | CB | TYR | A | 103 | −99.646 | 59.147 | 22.160 | 1.00 | 23.45 | C |
| ATOM | 61 | CG | TYR | A | 103 | −101.029 | 58.543 | 22.278 | 1.00 | 23.81 | C |
| ATOM | 62 | CD1 | TYR | A | 103 | −102.152 | 59.231 | 21.817 | 1.00 | 24.24 | C |
| ATOM | 63 | CE1 | TYR | A | 103 | −103.420 | 58.682 | 21.910 | 1.00 | 24.43 | C |
| ATOM | 64 | CZ | TYR | A | 103 | −103.579 | 57.423 | 22.464 | 1.00 | 24.46 | C |
| ATOM | 65 | OH | TYR | A | 103 | −104.840 | 56.896 | 22.551 | 1.00 | 24.90 | O |
| ATOM | 66 | CE2 | TYR | A | 103 | −102.482 | 56.714 | 22.929 | 1.00 | 24.26 | C |
| ATOM | 67 | CD2 | TYR | A | 103 | −101.215 | 57.274 | 22.828 | 1.00 | 24.19 | C |
| ATOM | 68 | C | TYR | A | 103 | −99.957 | 60.701 | 24.119 | 1.00 | 22.98 | C |
| ATOM | 69 | O | TYR | A | 103 | −99.518 | 59.912 | 24.954 | 1.00 | 22.67 | O |
| ATOM | 70 | N | GLN | A | 104 | −100.787 | 61.696 | 24.424 | 1.00 | 22.98 | N |
| ATOM | 71 | CA | GLN | A | 104 | −101.310 | 61.851 | 25.781 | 1.00 | 23.33 | C |
| ATOM | 72 | CB | GLN | A | 104 | −101.903 | 63.246 | 25.994 | 1.00 | 24.15 | C |
| ATOM | 73 | CG | GLN | A | 104 | −100.870 | 64.373 | 25.971 | 1.00 | 24.57 | C |
| ATOM | 74 | CD | GLN | A | 104 | −101.223 | 65.555 | 26.867 | 1.00 | 25.01 | C |
| ATOM | 75 | OE1 | GLN | A | 104 | −102.182 | 65.513 | 27.645 | 1.00 | 25.40 | O |
| ATOM | 76 | OE2 | GLN | A | 104 | −100.441 | 66.625 | 26.755 | 1.00 | 25.38 | N |
| ATOM | 77 | C | GLN | A | 104 | −102.340 | 60.776 | 26.126 | 1.00 | 22.63 | C |
| ATOM | 78 | O | GLN | A | 104 | −102.382 | 60.308 | 27.264 | 1.00 | 22.22 | O |
| ATOM | 79 | N | GLY | A | 105 | −103.161 | 60.388 | 25.150 | 1.00 | 22.16 | N |
| ATOM | 80 | CA | GLY | A | 105 | −104.205 | 59.389 | 25.362 | 1.00 | 21.43 | C |
| ATOM | 81 | C | GLY | A | 105 | −105.304 | 59.864 | 26.297 | 1.00 | 21.13 | C |
| ATOM | 82 | C | GLY | A | 105 | −105.446 | 61.062 | 26.544 | 1.00 | 21.02 | O |
| ATOM | 83 | N | SER | A | 106 | −106.063 | 58.912 | 26.834 | 1.00 | 20.71 | N |
| ATOM | 84 | CA | SER | A | 106 | −107.252 | 59.209 | 27.644 | 1.00 | 20.40 | C |
| ATOM | 85 | CB | SER | A | 106 | −108.169 | 57.983 | 27.707 | 1.00 | 20.49 | C |
| ATOM | 86 | OG | SER | A | 106 | −107.549 | 56.896 | 28.378 | 1.00 | 20.66 | O |
| ATOM | 87 | C | SER | A | 106 | −106.955 | 59.692 | 29.068 | 1.00 | 19.98 | C |
| ATOM | 88 | O | SER | A | 106 | −107.842 | 60.229 | 29.727 | 1.00 | 20.11 | O |
| ATOM | 89 | N | TYR | A | 107 | −105.728 | 59.491 | 29.546 | 1.00 | 19.73 | N |
| ATOM | 90 | CA | TYR | A | 107 | −105.333 | 59.925 | 30.891 | 1.00 | 19.40 | C |
| ATOM | 91 | CB | TYR | A | 107 | −104.541 | 58.806 | 31.577 | 1.00 | 19.74 | C |
| ATOM | 92 | CG | TYR | A | 107 | −105.360 | 57.543 | 31.731 | 1.00 | 19.91 | C |
| ATOM | 93 | CD1 | TYR | A | 107 | −106.302 | 57.420 | 32.753 | 1.00 | 20.16 | C |
| ATOM | 94 | CE1 | TYR | A | 107 | −107.072 | 56.724 | 32.885 | 1.00 | 20.24 | C |
| ATOM | 95 | CZ | TYR | A | 107 | −106.905 | 55.232 | 31.985 | 1.00 | 20.22 | C |
| ATOM | 96 | OH | TYR | A | 107 | −107.654 | 54.089 | 32.110 | 1.00 | 20.64 | O |
| ATOM | 97 | CE2 | TYR | A | 107 | −105.986 | 55.334 | 30.958 | 1.00 | 20.14 | C |
| ATOM | 98 | CD2 | TYR | A | 107 | −105.225 | 56.486 | 30.832 | 1.00 | 20.09 | C |
| ATOM | 99 | C | TYR | A | 107 | −104.556 | 61.244 | 30.926 | 1.00 | 18.78 | C |
| ATOM | 100 | O | TYR | A | 107 | −104.248 | 61.740 | 32.013 | 1.00 | 18.68 | O |
| ATOM | 101 | N | GLY | A | 108 | −104.253 | 61.818 | 29.760 | 1.00 | 18.27 | N |
| ATOM | 102 | CA | GLY | A | 108 | −103.518 | 63.084 | 29.688 | 1.00 | 18.06 | C |
| ATOM | 103 | C | GLY | A | 108 | −102.067 | 62.915 | 30.109 | 1.00 | 18.07 | C |
| ATOM | 104 | O | GLY | A | 108 | −101.552 | 63.695 | 30.911 | 1.00 | 17.80 | O |
| ATOM | 105 | N | PHE | A | 108 | −101.423 | 61.879 | 29.571 | 1.00 | 17.63 | N |
| ATOM | 106 | CA | PHE | A | 108 | −100.046 | 61.522 | 29.923 | 1.00 | 17.58 | C |
| ATOM | 107 | CG | PHE | A | 108 | −99.731 | 60.109 | 29.403 | 1.00 | 17.91 | C |
| ATOM | 108 | CG | PHE | A | 108 | −98.311 | 59.668 | 29.640 | 1.00 | 18.11 | C |
| ATOM | 109 | CD1 | PHE | A | 109 | −97.837 | 59.476 | 30.933 | 1.00 | 18.41 | C |
| ATOM | 110 | CE1 | PHE | A | 109 | −96.528 | 59.070 | 31.160 | 1.00 | 18.54 | C |
| ATOM | 111 | CZ | PHE | A | 109 | −95.680 | 58.842 | 30.088 | 1.00 | 18.61 | C |
| ATOM | 112 | CE2 | PHE | A | 109 | −96.141 | 59.021 | 28.791 | 1.00 | 18.47 | C |
| ATOM | 113 | CD2 | PHE | A | 109 | −97.450 | 59.432 | 28.572 | 1.00 | 18.37 | C |
| ATOM | 114 | C | PHE | A | 109 | −99.034 | 62.526 | 29.361 | 1.00 | 17.44 | C |
| ATOM | 115 | O | PHE | A | 109 | −98.928 | 62.673 | 28.147 | 1.00 | 17.13 | O |
| ATOM | 116 | N | ARG | A | 110 | −98.304 | 63.208 | 30.249 | 1.00 | 17.20 | N |
| ATOM | 117 | CA | ARG | A | 110 | −97.169 | 64.069 | 29.870 | 1.00 | 17.16 | C |
| ATOM | 118 | CB | ARG | A | 110 | −97.467 | 65.544 | 30.130 | 1.00 | 17.94 | C |
| ATOM | 119 | CG | ARG | A | 110 | −98.602 | 66.141 | 29.327 | 1.00 | 18.73 | C |
| ATOM | 120 | CD | ARG | A | 110 | −99.396 | 67.091 | 30.205 | 1.00 | 19.38 | C |
| ATOM | 121 | NE | ARG | A | 110 | −100.211 | 66.316 | 31.141 | 1.00 | 19.97 | N |
| ATOM | 122 | CZ | ARG | A | 110 | −100.656 | 66.727 | 32.328 | 1.00 | 20.01 | C |
| ATOM | 123 | NH1 | ARG | A | 110 | −101.389 | 65.893 | 33.049 | 1.00 | 19.77 | N |
| ATOM | 124 | NH2 | ARG | A | 110 | −100.376 | 67.938 | 32.815 | 1.00 | 20.24 | N |
| ATOM | 125 | C | ARG | A | 110 | −95.965 | 63.718 | 30.721 | 1.00 | 16.13 | C |
| ATOM | 126 | O | ARG | A | 110 | −96.099 | 63.071 | 31.756 | 1.00 | 15.80 | O |
| ATOM | 127 | N | LEU | A | 111 | −94.797 | 64.184 | 30.289 | 1.00 | 15.41 | N |
| ATOM | 128 | CA | LEU | A | 111 | −93.583 | 64.120 | 31.098 | 1.00 | 14.89 | C |
| ATOM | 129 | CB | LEU | A | 111 | −92.398 | 63.623 | 30.272 | 1.00 | 14.68 | C |
| ATOM | 130 | CG | LEU | A | 111 | −92.488 | 62.184 | 29.765 | 1.00 | 14.49 | C |
| ATOM | 131 | CD1 | LEU | A | 111 | −91.210 | 61.813 | 29.025 | 1.00 | 14.52 | C |
| ATOM | 132 | CD2 | LEU | A | 111 | −92.771 | 61.186 | 30.883 | 1.00 | 14.42 | C |
| ATOM | 133 | C | LEU | A | 111 | −93.262 | 65.488 | 31.687 | 1.00 | 14.70 | C |
| ATOM | 134 | O | LEU | A | 111 | −93.602 | 66.519 | 31.111 | 1.00 | 14.66 | O |
| ATOM | 135 | N | GLY | A | 112 | −92.606 | 65.466 | 32.841 | 1.00 | 14.43 | N |
| ATOM | 136 | CA | GLY | A | 112 | −92.120 | 66.662 | 33.521 | 1.00 | 14.51 | C |
| ATOM | 137 | C | GLY | A | 112 | −90.672 | 66.437 | 33.895 | 1.00 | 14.49 | C |
| ATOM | 138 | O | GLY | A | 112 | −90.227 | 65.289 | 34.034 | 1.00 | 13.91 | O |
| ATOM | 139 | N | PHE | A | 113 | −89.926 | 67.527 | 34.038 | 1.00 | 14.47 | N |
| ATOM | 140 | CA | PHE | A | 113 | −88.492 | 67.441 | 34.325 | 1.00 | 14.47 | C |
| ATOM | 141 | CB | PHE | A | 113 | −87.684 | 67.693 | 33.046 | 1.00 | 14.46 | C |
| ATOM | 142 | CG | PHE | A | 113 | −87.950 | 66.691 | 31.959 | 1.00 | 14.38 | C |
| ATOM | 143 | CD1 | PHE | A | 113 | −88.980 | 66.891 | 31.046 | 1.00 | 14.44 | C |
| ATOM | 144 | CE1 | PHE | A | 113 | −89.238 | 65.957 | 30.056 | 1.00 | 14.26 | C |
| ATOM | 145 | CZ | PHE | A | 113 | −88.464 | 64.811 | 29.968 | 1.00 | 14.40 | C |
| ATOM | 146 | CE2 | PHE | A | 113 | −87.441 | 64.595 | 30.878 | 1.00 | 14.33 | C |
| ATOM | 147 | CD2 | PHE | A | 113 | −87.193 | 65.527 | 31.867 | 1.00 | 14.36 | C |
| ATOM | 148 | C | PHE | A | 113 | −88.102 | 68.436 | 35.400 | 1.00 | 14.55 | C |
| ATOM | 149 | O | PHE | A | 113 | −88.798 | 69.437 | 35.613 | 1.00 | 14.66 | O |
| ATOM | 150 | N | LEU | A | 114 | −86.999 | 68.144 | 36.090 | 1.00 | 14.36 | N |
| ATOM | 151 | CA | LEU | A | 114 | −86.396 | 69.105 | 37.007 | 1.00 | 14.33 | C |
| ATOM | 152 | CB | LEU | A | 114 | −85.739 | 68.445 | 37.943 | 1.00 | 14.30 | C |
| ATOM | 153 | CG | LEU | A | 114 | −85.831 | 67.289 | 38.842 | 1.00 | 14.22 | C |
| ATOM | 154 | CD1 | LEU | A | 114 | −84.705 | 66.931 | 39.794 | 1.00 | 14.28 | C |
| ATOM | 155 | CD2 | LEU | A | 114 | −87.092 | 67.621 | 39.621 | 1.00 | 14.27 | C |
| ATOM | 156 | C | LEU | A | 114 | −85.700 | 70.180 | 36.192 | 1.00 | 14.40 | C |
| ATOM | 157 | O | LEU | A | 114 | −85.362 | 69.965 | 35.020 | 1.00 | 14.11 | O |
| ATOM | 158 | N | HIS | A | 115 | −85.498 | 71.329 | 36.830 | 1.00 | 14.50 | N |
| ATOM | 159 | CA | HIS | A | 115 | −84.817 | 74.472 | 36.238 | 1.00 | 14.91 | C |
| ATOM | 160 | CB | HIS | A | 115 | −85.760 | 73.676 | 36.208 | 1.00 | 15.07 | C |
| ATOM | 161 | CG | HIS | A | 115 | −87.078 | 73.383 | 35.559 | 1.00 | 15.28 | C |
| ATOM | 162 | ND1 | HIS | A | 115 | −88.277 | 73.493 | 36.228 | 1.00 | 15.70 | N |
| ATOM | 163 | CE1 | HIS | A | 115 | −89.262 | 73.162 | 35.416 | 1.00 | 15.77 | C |
| ATOM | 164 | NE2 | HIS | A | 115 | −88.748 | 72.823 | 34.249 | 1.00 | 15.95 | N |
| ATOM | 165 | CD2 | HIS | A | 115 | −87.381 | 72.945 | 34.315 | 1.00 | 15.85 | C |
| ATOM | 166 | C | HIS | A | 115 | −83.551 | 72.737 | 37.058 | 1.00 | 14.89 | C |
| ATOM | 167 | O | HIS | A | 115 | −83.475 | 73.689 | 37.834 | 1.00 | 14.78 | O |
| ATOM | 168 | N | HIS | A | 115 | −82.569 | 71.861 | 36.863 | 1.00 | 15.07 | N |
| ATOM | 169 | CA | SER | A | 116 | −81.372 | 71.776 | 37.715 | 1.00 | 15.35 | C |
| ATOM | 170 | CB | SER | A | 116 | −80.870 | 70.329 | 37.751 | 1.00 | 15.20 | C |
| ATOM | 171 | OG | SER | A | 116 | −81.864 | 69.470 | 38.261 | 1.00 | 15.40 | O |
| ATOM | 172 | C | SER | A | 116 | −80.207 | 72.665 | 37.310 | 1.00 | 15.56 | C |
| ATOM | 173 | O | SER | A | 116 | −79.186 | 72.689 | 38.016 | 1.00 | 15.61 | O |
| ATOM | 174 | N | GLY | A | 117 | −80.328 | 73.367 | 36.182 | 1.00 | 15.85 | N |
| ATOM | 175 | CA | GLY | A | 117 | −79.243 | 74.201 | 35.670 | 1.00 | 16.31 | C |
| ATOM | 176 | C | GLY | A | 117 | −78.035 | 73.413 | 35.182 | 1.00 | 16.67 | C |
| ATOM | 177 | O | GLY | A | 117 | −78.104 | 72.197 | 35.010 | 1.00 | 16.67 | O |
| ATOM | 178 | N | THR | A | 118 | −76.922 | 74.119 | 34.988 | 1.00 | 17.15 | N |
| ATOM | 179 | CA | THR | A | 118 | −75.729 | 73.564 | 34.341 | 1.00 | 17.52 | C |
| ATOM | 180 | CB | THR | A | 118 | −75.577 | 74.152 | 32.927 | 1.00 | 17.30 | C |
| ATOM | 181 | OG1 | THR | A | 118 | −75.417 | 75.571 | 33.011 | 1.00 | 17.46 | O |
| ATOM | 182 | OG2 | THR | A | 118 | −76.797 | 73.834 | 32.081 | 1.00 | 17.35 | C |
| ATOM | 183 | C | THR | A | 118 | −74.428 | 73.798 | 35.131 | 1.00 | 18.06 | C |
| ATOM | 184 | O | THR | A | 118 | −73.351 | 73.918 | 34.536 | 1.00 | 17.89 | O |
| ATOM | 185 | N | ALA | A | 119 | −74.528 | 73.838 | 36.462 | 1.00 | 18.66 | N |
| ATOM | 186 | CA | ALA | A | 119 | −73.358 | 74.032 | 37.333 | 1.00 | 19.48 | C |
| ATOM | 187 | CB | ALA | A | 119 | −73.799 | 74.296 | 38.768 | 1.00 | 19.28 | C |
| ATOM | 188 | C | ALA | A | 119 | −72.457 | 72.804 | 37.278 | 1.00 | 20.44 | C |
| ATOM | 189 | O | ALA | A | 119 | −72.929 | 71.709 | 36.996 | 1.00 | 20.37 | O |
| ATOM | 190 | N | LYS | A | 120 | −71.167 | 72.979 | 37.564 | 1.00 | 21.53 | N |
| ATOM | 191 | CA | LYS | A | 120 | −70.202 | 71.871 | 37.481 | 1.00 | 22.15 | C |
| ATOM | 192 | CB | LYS | A | 120 | −68.803 | 72.327 | 37.918 | 1.00 | 23.05 | C |
| ATOM | 193 | CG | LYS | A | 120 | −67.695 | 71.375 | 37.488 | 1.00 | 23.75 | C |
| ATOM | 194 | C | LYS | A | 120 | −66.351 | 71.743 | 38.092 | 1.00 | 24.24 | C |
| ATOM | 195 | CE | LYS | A | 120 | −65.372 | 70.594 | 37.938 | 1.00 | 24.70 | C |
| ATOM | 196 | NE | LYS | A | 120 | −63.974 | 70.962 | 38.290 | 1.00 | 25.08 | N |
| ATOM | 197 | C | LYS | A | 120 | −70.633 | 70.644 | 38.305 | 1.00 | 22.34 | C |
| ATOM | 198 | O | LYS | A | 120 | −70.451 | 69.504 | 37.874 | 1.00 | 22.46 | O |
| ATOM | 199 | N | SER | A | 121 | −71.217 | 70.896 | 39.473 | 1.00 | 22.24 | N |
| ATOM | 200 | CA | SER | A | 121 | −71.695 | 69.842 | 40.369 | 1.00 | 21.97 | C |
| ATOM | 201 | CB | SER | A | 121 | −71.942 | 70.437 | 41.757 | 1.00 | 22.42 | C |
| ATOM | 202 | OG | SER | A | 121 | −72.919 | 71.461 | 41.688 | 1.00 | 22.46 | O |
| ATOM | 203 | C | SER | A | 121 | −72.970 | 69.108 | 39.919 | 1.00 | 21.64 | C |
| ATOM | 204 | O | SER | A | 121 | −73.307 | 68.081 | 40.512 | 1.00 | 21.61 | O |
| ATOM | 205 | N | VAL | A | 122 | −73.686 | 69.622 | 38.913 | 1.00 | 21.11 | N |
| ATOM | 206 | CA | VAL | A | 122 | −74.905 | 68.962 | 38.426 | 1.00 | 20.74 | C |
| ATCN | 207 | CB | VAL | A | 122 | −75.791 | 69.895 | 37.545 | 1.00 | 20.91 | C |
| ATOM | 208 | CG1 | VAL | A | 122 | −75.235 | 70.084 | 36.133 | 1.00 | 20.95 | C |
| ATOM | 209 | CG2 | VAL | A | 122 | −77.218 | 69.367 | 37.469 | 1.00 | 20.99 | C |
| ATOM | 210 | C | VAL | A | 122 | −74.537 | 67.667 | 37.694 | 1.00 | 20.10 | C |
| ATOM | 211 | O | THR | A | 122 | −73.660 | 67.662 | 36.828 | 1.00 | 20.56 | O |
| ATOM | 212 | N | THR | A | 123 | −75.271 | 66.569 | 38.098 | 1.00 | 19.17 | N |
| ATOM | 213 | CA | THR | A | 123 | −74.959 | 65.261 | 37.484 | 1.00 | 19.09 | C |
| ATOM | 214 | CB | THR | A | 123 | −74.804 | 64.177 | 38.565 | 1.00 | 18.89 | C |
| ATOM | 215 | OG1 | THR | A | 123 | −75.995 | 64.116 | 39.356 | 1.00 | 18.78 | O |
| ATOM | 216 | CG2 | THR | A | 123 | −73.617 | 64.491 | 39.476 | 1.00 | 19.14 | C |
| ATOM | 217 | C | THR | A | 123 | −76.095 | 64.868 | 36.528 | 1.00 | 18.85 | C |
| ATOM | 218 | O | THR | A | 123 | −75.970 | 63.887 | 35.793 | 1.00 | 18.92 | O |
| ATOM | 219 | N | CYS | A | 124 | −77.198 | 65.617 | 36.552 | 1.00 | 19.06 | N |
| ATOM | 220 | CA | CYS | A | 124 | −78.333 | 65.380 | 35.657 | 1.00 | 19.13 | C |
| ATOM | 221 | CB | CYS | A | 124 | −79.262 | 64.332 | 36.225 | 1.00 | 20.01 | C |
| ATOM | 222 | SG | CYS | A | 124 | −80.724 | 63.995 | 35.247 | 1.00 | 21.65 | S |
| ATOM | 223 | C | CYS | A | 124 | −79.106 | 66.675 | 35.397 | 1.00 | 18.26 | C |
| ATOM | 224 | O | CYS | A | 124 | −79.491 | 67.369 | 36.342 | 1.00 | 18.58 | O |
| ATOM | 225 | N | THR | A | 125 | −79.337 | 66.986 | 34.120 | 1.00 | 17.40 | N |
| ATOM | 226 | CA | THR | A | 125 | −79.929 | 68.275 | 33.725 | 1.00 | 16.72 | C |
| ATOM | 227 | CB | THR | A | 125 | −78.884 | 69.417 | 33.774 | 1.00 | 16.56 | C |
| ATOM | 228 | OG1 | THR | A | 125 | −79.464 | 70.644 | 33.310 | 1.00 | 16.21 | O |
| ATOM | 229 | CG2 | THR | A | 125 | −77.644 | 69.088 | 32.947 | 1.00 | 16.54 | C |
| ATOM | 230 | C | THR | A | 125 | −80.610 | 68.208 | 32.350 | 1.00 | 16.11 | C |
| ATOM | 231 | O | THR | A | 125 | −80.073 | 67.624 | 31.406 | 1.00 | 16.06 | O |
| ATOM | 232 | N | TYR | A | 126 | −81.794 | 68.814 | 32.266 | 1.00 | 15.27 | N |
| ATOM | 233 | CA | TYR | A | 126 | −82.659 | 68.744 | 31.087 | 1.00 | 14.80 | C |
| ATOM | 234 | CB | TYR | A | 126 | −84.086 | 684.26 | 31.532 | 1.00 | 14.70 | C |
| ATOM | 235 | CG | TYR | A | 126 | −85.141 | 68.499 | 30.446 | 1.00 | 14.53 | C |
| ATOM | 236 | CD1 | TYR | A | 126 | −85.173 | 67.570 | 29.405 | 1.00 | 14.81 | C |
| ATOM | 237 | CE1 | TYR | A | 126 | −86.155 | 67.632 | 28.416 | 1.00 | 14.79 | C |
| ATOM | 238 | CZ | TYR | A | 126 | −87.117 | 68.622 | 28.474 | 1.00 | 14.80 | C |
| ATOM | 239 | OH | TYR | A | 126 | −86.093 | 6.8702 | 27.508 | 1.00 | 15.34 | O |
| ATOM | 240 | CE2 | TYR | A | 126 | −87.112 | 69.547 | 29.501 | 1.00 | 14.56 | C |
| ATOM | 241 | CD2 | TYR | A | 126 | −86.133 | 69.479 | 30.480 | 1.00 | 14.60 | C |
| ATOM | 242 | C | TYR | A | 126 | −82.638 | 70.078 | 30.350 | 1.00 | 14.56 | C |
| ATOM | 243 | O | TYR | A | 126 | −82.813 | 71.124 | 30.968 | 1.00 | 14.49 | O |
| ATOM | 244 | N | SER | A | 127 | −82.415 | 70.031 | 29.039 | 1.00 | 14.34 | N |
| ATOM | 245 | CA | SER | A | 127 | −82.564 | 71.203 | 28.177 | 1.00 | 14.23 | C |
| ATOM | 246 | CB | SER | A | 127 | −81.580 | 71.154 | 27.011 | 1.00 | 14.13 | C |
| ATOM | 247 | OG | SER | A | 127 | −81.854 | 72.187 | 26.077 | 1.00 | 14.00 | O |
| ATOM | 248 | C | SER | A | 127 | −83.996 | 71.220 | 27.626 | 1.00 | 14.15 | C |
| ATOM | 249 | O | SER | A | 127 | −84.356 | 70.327 | 26.855 | 1.00 | 13.80 | O |
| ATOM | 250 | N | PRO | A | 128 | −84.819 | 72.209 | 28.037 | 1.00 | 14.22 | N |
| ATOM | 251. | CA | PRO | A | 128 | −86.135 | 72.349 | 27.392 | 1.00 | 14.43 | C |
| ATOM | 252 | CB | PRO | A | 128 | −86.862 | 73.379 | 28.265 | 1.00 | 14.31 | C |
| ATOM | 253 | CG | PRO | A | 128 | −85.812 | 74.039 | 29.077 | 1.00 | 14.22 | C |
| ATOM | 254 | CD | PRO | A | 128 | −84.723 | 73.035 | 29.254 | 1.00 | 14.12 | C |
| ATOM | 255 | C | PRO | A | 128 | −86.060 | 72.811 | 25.933 | 1.00 | 14.50 | C |
| ATOM | 256 | O | PRO | A | 128 | −86.922 | 72.443 | 25.144 | 1.00 | 14.79 | O |
| ATOM | 257 | N | ALA | A | 129 | −85.046 | 73.600 | 25.583 | 1.00 | 14.78 | N |
| ATOM | 258 | CA | ALA | A | 129 | −84.843 | 74.037 | 24.196 | 1.00 | 15.09 | C |
| ATOM | 259 | CB | ALA | A | 129 | −83.828 | 75.170 | 24.140 | 1.00 | 15.14 | C |
| ATOM | 260 | C | ALA | A | 129 | −84.423 | 72.894 | 23.255 | 1.00 | 15.26 | C |
| ATOM | 261 | O | ALA | A | 129 | −84.697 | 72.953 | 22.056 | 1.00 | 15.80 | O |
| ATOM | 262 | N | LEU | A | 130 | −83.761 | 71.869 | 23.795 | 1.00 | 15.21 | N |
| ATOM | 263 | CA | LEU | A | 130 | −83.360 | 70.682 | 23.023 | 1.00 | 15.13 | C |
| ATOM | 264 | CB | LEU | A | 130 | −81.890 | 70.356 | 23.312 | 1.00 | 15.17 | C |
| ATOM | 265 | CG | LEU | A | 130 | −80.838 | 71.383 | 22.890 | 1.00 | 15.25 | C |
| ATOM | 266 | CD1 | LEU | A | 130 | −79.624 | 71.340 | 23.809 | 1.00 | 15.45 | C |
| ATOM | 267 | CD2 | LEU | A | 130 | −80.410 | 71.141 | 21.450 | 1.00 | 15.34 | C |
| ATOM | 268 | C | LEU | A | 130 | −84.208 | 69.431 | 23.317 | 1.00 | 15.14 | C |
| ATOM | 269 | O | LEU | A | 130 | −84.022 | 68.394 | 22.655 | 1.00 | 15.22 | O |
| ATOM | 270 | N | ASN | A | 131 | −85.120 | 69.515 | 24.292 | 1.00 | 14.91 | N |
| ATOM | 271 | CA | ASN | A | 131 | −85.840 | 63.343 | 24.827 | 1.00 | 14.94 | C |
| ATOM | 272 | CB | ASN | A | 131 | −87.037 | 67.967 | 23.935 | 1.00 | 14.97 | C |
| ATOM | 273 | CG | ASN | A | 131 | −87.871 | 66.831 | 24.510 | 1.00 | 15.05 | C |
| ATOM | 274 | OD1 | ASN | A | 131 | −88.020 | 66.708 | 25.726 | 1.00 | 15.39 | O |
| ATOM | 275 | ND2 | ASN | A | 131 | −88.394 | 65.892 | 23.633 | 1.00 | 15.07 | N |
| ATOM | 276 | C | ASN | A | 131 | −84.866 | 67.170 | 25.020 | 1.00 | 14.96 | C |
| ATOM | 277 | O | ASN | A | 131 | −85.053 | 66.062 | 24.493 | 1.00 | 14.38 | O |
| ATOM | 278 | N | LYS | A | 132 | −83.798 | 67.459 | 25.757 | 1.00 | 15.15 | N |
| ATOM | 279 | CA | LYS | A | 132 | −82.661 | 66.567 | 25.843 | 1.00 | 15.72 | C |
| ATOM | 280 | CB | LYS | A | 132 | −81.584 | 66.961 | 24.831 | 1.00 | 15.86 | C |
| ATOM | 281 | CG | LYS | A | 132 | −80.348 | 66.073 | 24.877 | 1.00 | 15.82 | C |
| ATOM | 282 | CD | LYS | A | 132 | −79.384 | 66.365 | 23.738 | 1.00 | 15.80 | C |
| ATOM | 283 | CE | LYS | A | 132 | −78.186 | 65.429 | 23.792 | 1.00 | 15.92 | C |
| ATOM | 284 | NZ | LYS | A | 132 | −77.509 | 65.231 | 22.478 | 1.00 | 15.93 | N |
| ATOM | 285 | C | LYS | A | 132 | −82.075 | 66.581 | 27.237 | 1.00 | 16.24 | C |
| ATOM | 286 | O | LYS | A | 132 | −81.748 | 67.641 | 27.768 | 1.00 | 16.06 | O |
| ATOM | 287 | N | MET | A | 133 | −81.932 | 65.382 | 27.797 | 1.00 | 17.05 | N |
| ATOM | 288 | CA | MET | A | 133 | −81.354 | 65.173 | 29.103 | 1.00 | 17.78 | C |
| ATOM | 289 | CB | MET | A | 133 | −82.020 | 63.958 | 29.765 | 1.00 | 18.71 | C |
| ATOM | 290 | CG | MET | A | 133 | −81.790 | 63.840 | 31.264 | 1.00 | 19.37 | C |
| ATOM | 291 | SD | MET | A | 133 | −82.730 | 64.977 | 32.298 | 1.00 | 20.84 | S |
| ATOM | 292 | CE | MET | A | 133 | −84.032 | 63.923 | 32.935 | 1.00 | 20.38 | C |
| ATOM | 293 | C | MET | A | 133 | −79.846 | 64.957 | 28.938 | 1.00 | 17.88 | C |
| ATOM | 294 | O | MET | A | 133 | −79.392 | 64.334 | 27.966 | 1.00 | 17.35 | O |
| ATOM | 295 | N | PHE | A | 134 | −79.082 | 65.506 | 29.877 | 1.00 | 18.10 | N |
| ATOM | 296 | CA | PHE | A | 134 | −77.640 | 65.315 | 29.951 | 1.00 | 18.42 | C |
| ATOM | 297 | CB | PHE | A | 134 | −76.909 | 66.652 | 29.804 | 1.00 | 18.05 | C |
| ATOM | 298 | CG | PHE | A | 134 | −77.057 | 67.284 | 28.444 | 1.00 | 17.69 | C |
| ATOM | 299 | CD1 | PHE | A | 134 | −78.241 | 67.927 | 28.083 | 1.00 | 17.51 | C |
| ATOM | 300 | CE1 | PHE | A | 134 | −78.377 | 68.522 | 26.836 | 1.00 | 17.36 | C |
| ATOM | 301 | CZ | PHE | A | 134 | −77.329 | 68.478 | 25.929 | 1.00 | 17.28 | C |
| ATOM | 302 | CE2 | PHE | A | 134 | −76.143 | 67.847 | 26.274 | 1.00 | 17.33 | C |
| ATOM | 303 | CD2 | PHE | A | 134 | −76.010 | 67.254 | 27.524 | 1.00 | 17.61 | C |
| ATOM | 304 | C | PHE | A | 134 | −77.360 | 64.687 | 31.311 | 1.00 | 19.17 | C |
| ATOM | 305 | O | PHE | A | 134 | −77.693 | 65.278 | 32.336 | 1.00 | 18.76 | O |
| ATOM | 306 | N | CYS | A | 135 | −76.772 | 63.489 | 31.314 | 1.00 | 20.84 | N |
| ATOM | 307 | CA | CYS | A | 135 | −76.604 | 62.691 | 32.533 | 1.00 | 22.25 | C |
| ATOM | 308 | CB | CYS | A | 135 | −77.531 | 61.471 | 32.501 | 1.00 | 23.89 | C |
| ATOM | 309 | SG | CYS | A | 135 | −79.245 | 61.795 | 32.030 | 1.00 | 27.30 | S |
| ATOM | 310 | C | CYS | A | 135 | −75.173 | 62.187 | 32.689 | 1.00 | 21.84 | C |
| ATOM | 311 | O | CYS | A | 135 | −74.543 | 61.798 | 31.712 | 1.00 | 21.38 | O |
| ATOM | 312 | N | GLN | A | 136 | −74.666 | 62.183 | 33.920 | 1.00 | 21.77 | N |
| ATOM | 313 | CA | GLN | A | 136 | −73.467 | 61.396 | 34.240 | 1.00 | 21.82 | C |
| ATOM | 314 | CB | GLN | A | 136 | −72.822 | 61.839 | 35.557 | 1.00 | 21.72 | C |
| ATOM | 315 | CG | GLN | A | 136 | −71.915 | 63.055 | 35.436 | 1.00 | 21.88 | C |
| ATOM | 316 | CD | GLN | A | 136 | −71.363 | 63.521 | 36.775 | 1.00 | 22.03 | C |
| ATOM | 317 | OE1 | GLN | A | 136 | −71.486 | 62.832 | 37.788 | 1.00 | 22.47 | O |
| ATOM | 318 | NE2 | GLN | A | 136 | −70.753 | 64.698 | 36.783 | 1.00 | 22.05 | N |
| ATOM | 319 | C | GLN | A | 136 | −73.871 | 59.923 | 34.291 | 1.00 | 21.68 | C |
| ATOM | 320 | O | GLN | A | 136 | −75.023 | 59.594 | 34.595 | 1.00 | 21.46 | O |
| ATOM | 321 | N | LEU | A | 137 | −72.921 | 59.046 | 33.986 | 1.00 | 21.81 | N |
| ATOM | 322 | CA | LEU | A | 137 | −73.199 | 57.617 | 33.849 | 1.00 | 21.92 | C |
| ATOM | 323 | CB | LEU | A | 137 | −72.010 | 56.897 | 33.198 | 1.00 | 22.18 | C |
| ATOM | 324 | CG | LEU | A | 137 | −72.316 | 55.532 | 32.566 | 1.00 | 22.42 | C |
| ATOM | 325 | CD1 | LEU | A | 137 | −71.504 | 55.346 | 31.293 | 1.00 | 22.48 | C |
| ATOM | 326 | CD2 | LEU | A | 137 | −72.082 | 54.379 | 33.533 | 1.00 | 22.61 | C |
| ATOM | 327 | C | LEU | A | 137 | −73.534 | 57.001 | 35.209 | 1.00 | 21.82 | C |
| ATOM | 328 | O | LEU | A | 137 | −72.835 | 57.245 | 36.193 | 1.00 | 21.50 | O |
| ATOM | 329 | N | ALA | A | 138 | −74.622 | 56.228 | 35.248 | 1.00 | 21.58 | N |
| ATOM | 330 | CA | ALA | A | 138 | −75.117 | 55.567 | 36.472 | 1.00 | 21.47 | C |
| ATOM | 331 | CB | ALA | A | 138 | −74.902 | 54.559 | 36.990 | 1.00 | 21.37 | C |
| ATOM | 332 | C | ALA | A | 138 | −75.550 | 56.518 | 37.601 | 1.00 | 21.41 | C |
| ATOM | 333 | O | ALA | A | 138 | −75.548 | 56.135 | 38.773 | 1.00 | 21.95 | O |
| ATOM | 334 | N | LYS | A | 139 | −75.935 | 57.742 | 37.250 | 1.00 | 21.11 | N |
| ATOM | 335 | CA | LYS | A | 139 | −76.411 | 58.713 | 38.230 | 1.00 | 20.94 | C |
| ATOM | 336 | CB | LYS | A | 139 | −75.778 | 60.077 | 37.978 | 1.00 | 21.15 | C |
| ATOM | 337 | CG | LYS | A | 139 | −74.252 | 60.082 | 38.040 | 1.00 | 21.34 | C |
| ATOM | 338 | CD | LYS | A | 139 | −73.711 | 59.425 | 39.302 | 1.00 | 21.54 | C |
| ATOM | 339 | CE | LYS | A | 139 | −72.256 | 59.787 | 39.555 | 1.00 | 21.78 | C |
| ATOM | 340 | NZ | LYS | A | 139 | −71.853 | 59.373 | 40.294 | 1.00 | 21.89 | N |
| ATOM | 341 | C | LYS | A | 139 | −77.921 | 58.798 | 38.154 | 1.00 | 20.81 | C |
| ATOM | 342 | O | LYS | A | 139 | −78.523 | 58.362 | 37.170 | 1.00 | 20.96 | O |
| ATOM | 343 | N | THR | A | 140 | −78.529 | 59.343 | 39.205 | 1.00 | 20.70 | N |
| ATOM | 344 | CA | THR | A | 140 | −79.982 | 59.445 | 39.284 | 1.00 | 20.69 | C |
| ATOM | 345 | CB | THR | A | 140 | −80.454 | 59.895 | 40.680 | 1.00 | 20.56 | C |
| ATOM | 346 | OG1 | THR | A | 140 | −79.930 | 59.004 | 41.671 | 1.00 | 20.31 | O |
| ATOM | 347 | CG2 | THR | A | 140 | −81.980 | 59.895 | 40.768 | 1.00 | 20.56 | C |
| ATOM | 348 | C | THR | A | 140 | −80.487 | 60.407 | 38.210 | 1.00 | 20.82 | C |
| ATOM | 349 | O | THR | A | 140 | −79.927 | 61.486 | 38.014 | 1.00 | 20.69 | O |
| ATOM | 350 | N | CYS | A | 141 | −81.537 | 59.985 | 37.511 | 1.00 | 20.81 | N |
| ATOM | 351 | CA | CYS | A | 141 | −82.087 | 60.725 | 36.392 | 1.00 | 20.97 | C |
| ATOM | 352 | CB | CYS | A | 141 | −81.673 | 60.054 | 35.078 | 1.00 | 22.35 | C |
| ATOM | 353 | SG | CYS | A | 141 | −81.627 | 61.191 | 33.683 | 1.00 | 25.00 | S |
| ATOM | 354 | C | CYS | A | 141 | −83.607 | 60.803 | 36.555 | 1.00 | 19.87 | C |
| ATOM | 355 | O | CYS | A | 141 | −84.329 | 59.960 | 36.021 | 1.00 | 19.31 | O |
| ATOM | 356 | N | PRO | A | 142 | −84.093 | 61.802 | 37.330 | 1.00 | 18.88 | N |
| ATOM | 357 | CA | PRO | A | 142 | −85.525 | 61.923 | 37.600 | 1.00 | 18.34 | C |
| ATOM | 358 | CB | PRO | A | 142 | −85.611 | 63.106 | 38.584 | 1.00 | 18.60 | C |
| ATOM | 359 | CG | PRO | A | 142 | −84.241 | 63.260 | 39.142 | 1.00 | 18.84 | C |
| ATOM | 360 | CD | PRO | A | 142 | −83.319 | 62.836 | 38.046 | 1.00 | 18.90 | C |
| ATOM | 361 | C | PRO | A | 142 | −86.339 | 62.226 | 36.347 | 1.00 | 17.73 | C |
| ATOM | 362 | O | PRO | A | 142 | −85.980 | 63.127 | 35.582 | 1.00 | 17.99 | O |
| ATOM | 363 | N | VAL | A | 143 | −87.408 | 61.463 | 36.140 | 1.00 | 16.79 | N |
| ATOM | 364 | CA | VAL | A | 143 | −88.371 | 61.724 | 35.068 | 1.00 | 16.23 | C |
| ATOM | 365 | CB | VAL | A | 143 | −88.269 | 60.688 | 33.924 | 1.00 | 16.14 | C |
| ATOM | 366 | 001 | VAL | A | 143 | −89.315 | 60.965 | 32.846 | 1.00 | 16.15 | C |
| ATOM | 367 | CG2 | VAL | A | 143 | −86.870 | 60.699 | 33.317 | 1.00 | 16.16 | C |
| ATOM | 368 | C | VAL | A | 143 | −89.761 | 61.699 | 35.695 | 1.00 | 15.85 | C |
| ATOM | 369 | O | VAL | A | 143 | −90.160 | 60.690 | 36.277 | 1.00 | 15.81 | O |
| ATOM | 370 | N | GLN | A | 144 | −90.479 | 62.816 | 35.592 | 1.00 | 15.38 | N |
| ATOM | 371 | CA | GLN | A | 144 | −91.808 | 62.944 | 36.195 | 1.00 | 15.13 | C |
| ATOM | 372 | CB | GLN | A | 144 | −92.097 | 64.387 | 36.605 | 1.00 | 14.86 | C |
| ATOM | 373 | CG | GLN | A | 144 | −91.127 | 64.982 | 37.614 | 1.00 | 14.56 | C |
| ATOM | 374 | CD | GLN | A | 144 | −91.244 | 66.497 | 37.724 | 1.00 | 14.42 | C |
| ATOM | 375 | OE1 | GLN | A | 144 | −92.112 | 67.117 | 37.111 | 1.00 | 13.84 | O |
| ATOM | 376 | OE2 | GLN | A | 144 | −90.363 | 67.097 | 38.516 | 1.00 | 14.56 | N |
| ATOM | 377 | C | GLN | A | 144 | −92.858 | 62.514 | 35.194 | 1.00 | 15.01 | C |
| ATOM | 378 | O | GLN | A | 144 | −92.754 | 62.854 | 34.020 | 1.00 | 15.07 | O |
| ATOM | 379 | N | LEU | A | 145 | −93.857 | 61.771 | 35.666 | 1.00 | 15.11 | N |
| ATOM | 380 | CA | LEU | A | 145 | −95.039 | 61.437 | 34.880 | 1.00 | 15.11 | C |
| ATOM | 381 | CB | LEU | A | 145 | −95.393 | 59.957 | 35.014 | 1.00 | 14.89 | C |
| ATOM | 382 | CG | LEU | A | 145 | −94.278 | 58.919 | 34.973 | 1.00 | 14.75 | C |
| ATOM | 383 | 001 | LEU | A | 145 | −94.897 | 57.542 | 35.126 | 1.00 | 14.70 | C |
| ATOM | 384 | 002 | LEU | A | 145 | −93.463 | 59.009 | 33.694 | 1.00 | 14.72 | C |
| ATOM | 385 | C | LEU | A | 145 | −96.200 | 62.262 | 35.408 | 1.00 | 15.26 | C |
| ATOM | 386 | O | LEU | A | 145 | −96.487 | 62.231 | 36.607 | 1.00 | 15.25 | O |
| ATOM | 387 | N | TRP | A | 146 | −96.863 | 62.982 | 34.512 | 1.00 | 15.48 | N |
| ATOM | 388 | CA | TRP | A | 146 | −98.018 | 63.796 | 34.855 | 1.00 | 15.94 | C |
| ATOM | 389 | CB | TRP | A | 146 | −97.786 | 65.249 | 34.442 | 1.00 | 16.03 | C |
| ATOM | 390 | CG | TRP | A | 146 | −96.762 | 65.968 | 35.279 | 1.00 | 16.22 | C |
| ATOM | 391 | CD1 | TRP | A | 146 | −95.407 | 65.804 | 35.245 | 1.00 | 16.33 | C |
| ATOM | 392 | NE1 | TRP | A | 146 | −94.801 | 66.645 | 36.153 | 1.00 | 16.38 | N |
| ATOM | 393 | CE2 | TRP | A | 146 | −95.768 | 67.374 | 36.792 | 1.00 | 16.53 | C |
| ATOM | 394 | CD2 | TRP | A | 146 | −97.019 | 66.975 | 36.264 | 1.00 | 16.54 | C |
| ATOM | 395 | CE3 | TRP | A | 146 | −97.184 | 67.576 | 36.757 | 1.00 | 16.72 | C |
| ATOM | 396 | CZ3 | TRP | A | 146 | −98.067 | 68.547 | 37.746 | 1.00 | 16.85 | C |
| ATOM | 397 | CH2 | TRP | A | 146 | −96.807 | 68.921 | 38.252 | 1.00 | 16.90 | C |
| ATOM | 398 | CZ2 | TRP | A | 146 | −95.651 | 68.344 | 37.790 | 1.00 | 16.78 | C |
| ATOM | 399 | C | TRP | A | 146 | −99.232 | 63.227 | 34.130 | 1.00 | 16.08 | C |
| ATOM | 400 | O | TRP | A | 146 | −99.135 | 62.826 | 32.967 | 1.00 | 15.66 | O |
| ATOM | 401 | N | VAL | A | 147 | −100.363 | 63.190 | 34.828 | 1.00 | 16.65 | N |
| ATOM | 402 | CA | VAL | A | 147 | −101.623 | 62.684 | 34.278 | 1.00 | 17.20 | C |
| ATOM | 403 | CB | VAL | A | 147 | −101.933 | 61.249 | 34.756 | 1.00 | 17.09 | C |
| ATOM | 404 | CG1 | VAL | A | 147 | −100.964 | 60.260 | 34.135 | 1.00 | 17.13 | C |
| ATOM | 405 | CG2 | VAL | A | 147 | −101.913 | 61.133 | 36.281 | 1.00 | 17.24 | C |
| ATOM | 406 | C | VAL | A | 147 | −102.772 | 63.602 | 34.673 | 1.00 | 17.92 | C |
| ATOM | 407 | O | VAL | A | 147 | −102.718 | 34.246 | 35.718 | 1.00 | 18.66 | O |
| ATOM | 408 | N | ASP | A | 148 | −103.786 | 63.677 | 33.815 | 1.00 | 18.54 | N |
| ATOM | 409 | CA | ASP | A | 148 | −105.033 | 64.378 | 34.125 | 1.00 | 18.97 | C |
| ATOM | 410 | CB | ASP | A | 148 | −105.647 | 64.977 | 32.854 | 1.00 | 19.12 | C |
| ATOM | 411 | CG | ASP | A | 148 | −104.753 | 66.033 | 32.208 | 1.00 | 19.25 | C |
| ATOM | 412 | OD1 | ASP | A | 148 | −104.013 | 66.737 | 32.933 | 1.00 | 19.04 | O |
| ATOM | 413 | OD2 | ASP | A | 148 | −104.789 | 66.161 | 30.967 | 1.00 | 19.56 | O |
| ATOM | 414 | C | ASP | A | 148 | −106.021 | 63.447 | 34.827 | 1.00 | 19.52 | C |
| ATOM | 415 | O | ASP | A | 148 | −106.790 | 63.898 | 35.675 | 1.00 | 19.23 | O |
| ATOM | 416 | N | SER | A | 149 | −105.996 | 62.158 | 34.472 | 1.00 | 20.52 | N |
| ATOM | 417 | CA | SER | A | 149 | −106.761 | 61.114 | 35.162 | 1.00 | 21.22 | C |
| ATOM | 418 | CB | SER | A | 149 | −107.820 | 60.521 | 34.230 | 1.00 | 21.57 | C |
| ATOM | 419 | OG | SER | A | 149 | −108.926 | 61.388 | 34.125 | 1.00 | 22.78 | O |
| ATOM | 420 | C | SER | A | 149 | −105.831 | 60.003 | 35.622 | 1.00 | 21.38 | C |
| ATOM | 421 | O | SER | A | 149 | −104.910 | 59.638 | 34.905 | 1.00 | 21.44 | O |
| ATOM | 422 | N | THR | A | 150 | −106.096 | 594.54 | 36.804 | 1.00 | 21.90 | N |
| ATOM | 423 | CA | THR | A | 150 | −105.252 | 58.416 | 37.396 | 1.00 | 22.37 | C |
| ATOM | 424 | CB | THR | A | 150 | −105.591 | 58.206 | 38.891 | 1.00 | 22.87 | C |
| ATOM | 425 | OG1 | THR | A | 150 | −105.603 | 59.472 | 39.565 | 1.00 | 22.59 | O |
| ATOM | 426 | CG2 | THR | A | 150 | −104.574 | 57.278 | 39.573 | 1.00 | 22.86 | C |
| ATOM | 427 | C | THR | A | 150 | −105.453 | 57.094 | 36.640 | 1.00 | 22.58 | C |
| ATOM | 428 | O | THR | A | 150 | −106.569 | 56.577 | 36.613 | 1.00 | 22.73 | O |
| ATOM | 429 | N | PRO | A | 151 | −104.386 | 56.535 | 36.006 | 1.00 | 22.69 | N |
| ATOM | 430 | CA | PRO | A | 151 | −104.502 | 552.16 | 35.406 | 1.00 | 23.01 | C |
| ATOM | 431 | CB | PRO | A | 151 | −103.132 | 54.997 | 34.738 | 1.00 | 23.07 | C |
| ATOM | 432 | CG | PRO | A | 151 | −102.497 | 56.338 | 34.660 | 1.00 | 22.94 | C |
| ATOM | 433 | CD | PRO | A | 151 | −103.040 | 57.122 | 35.811 | 1.00 | 22.70 | C |
| ATOM | 434 | C | PRO | A | 151 | −104.752 | 54.150 | 36.478 | 1.00 | 23.14 | C |
| ATOM | 435 | O | PRO | A | 151 | −104.299 | 54.322 | 37.605 | 1.00 | 23.51 | O |
| ATOM | 436 | N | PRO | A | 152 | −105.457 | 53.052 | 36.136 | 1.00 | 23.44 | N |
| ATOM | 437 | CA | PRO | A | 152 | −105.814 | 52−082 | 37.186 | 1.00 | 23.44 | C |
| ATOM | 438 | CB | PRO | A | 152 | −106.834 | 51.161 | 36.500 | 1.00 | 23.61 | C |
| ATOM | 439 | CG | PRO | A | 152 | −106.695 | 51.388 | 35.033 | 1.00 | 23.66 | C |
| ATOM | 440 | CD | PRO | A | 152 | −105.834 | 52.591 | 34.786 | 1.00 | 23.67 | C |
| ATOM | 441 | C | PRO | A | 152 | −104.618 | 51.285 | 37.736 | 1.00 | 23.21 | C |
| ATOM | 442 | O | PRO | A | 152 | −103.512 | 51.410 | 37.210 | 1.00 | 23.17 | O |
| ATOM | 443 | N | PRO | A | 153 | −104.830 | 50.487 | 38.806 | 1.00 | 23.12 | N |
| ATOM | 444 | CA | PRO | A | 153 | −103.747 | 49.628 | 39.301 | 1.00 | 22.69 | C |
| ATOM | 445 | CB | PRO | A | 153 | −104.349 | 48.963 | 40.549 | 1.00 | 22.96 | C |
| ATOM | 446 | CG | PRO | A | 153 | −105.515 | 49.807 | 40.924 | 1.00 | 23.39 | C |
| ATOM | 447 | CD | PRO | A | 153 | −106.041 | 50.368 | 39.638 | 1.00 | 23.32 | C |
| ATOM | 448 | C | PRO | A | 153 | −103.354 | 48.577 | 38.265 | 1.00 | 21.84 | C |
| ATOM | 449 | O | PRO | A | 153 | −104.214 | 48.081 | 37.532 | 1.00 | 21.87 | O |
| ATOM | 450 | N | GLY | A | 154 | −102.063 | 48.267 | 38.195 | 1.00 | 21.16 | N |
| ATOM | 451 | CA | GLY | A | 154 | −101.534 | 47.339 | 37.194 | 1.00 | 20.47 | C |
| ATOM | 452 | C | GLY | A | 154 | −101.052 | 47.991 | 35.906 | 1.00 | 19.84 | C |
| ATOM | 453 | O | GLY | A | 154 | −100.548 | 47.297 | 35.021 | 1.00 | 19.87 | O |
| ATOM | 454 | N | THR | A | 155 | −101.211 | 49.312 | 35.789 | 1.00 | 19.39 | N |
| ATOM | 455 | CA | THR | A | 155 | −100.635 | 50.079 | 34.685 | 1.00 | 19.01 | C |
| ATOM | 456 | CB | THR | A | 155 | −101.191 | 51.525 | 34.644 | 1.00 | 19.35 | C |
| ATOM | 457 | OG1 | THR | A | 155 | −102.623 | 51.491 | 34.669 | 1.00 | 19.79 | O |
| ATOM | 458 | OG2 | THR | A | 155 | −100.751 | 52.265 | 33.390 | 1.00 | 19.53 | C |
| ATOM | 459 | C | THR | A | 155 | −99.122 | 50.112 | 34.869 | 1.00 | 18.36 | C |
| ATOM | 460 | O | THR | A | 155 | −98.630 | 50.140 | 35.995 | 1.00 | 18.36 | O |
| ATOM | 461 | N | ARG | A | 156 | −98.395 | 50.105 | 33.758 | 1.00 | 17.94 | N |
| ATOM | 462 | CA | ARG | A | 156 | −96.943 | 49.981 | 33.764 | 1.00 | 17.47 | C |
| ATOM | 463 | CB | ARG | A | 156 | −95.557 | 48.569 | 33.299 | 1.00 | 17.31 | C |
| ATOM | 464 | CG | ARG | A | 156 | −96.578 | 47.537 | 34.426 | 1.00 | 17.29 | C |
| ATOM | 465 | CD | ARG | A | 156 | −97.113 | 46.182 | 33.994 | 1.00 | 17.38 | C |
| ATOM | 466 | NE | ARG | A | 156 | −96.336 | 45.559 | 32.904 | 1.00 | 17.65 | N |
| ATOM | 467 | CZ | ARG | A | 156 | −95.175 | 44.953 | 33.032 | 1.00 | 17.66 | C |
| ATOM | 468 | NH1 | ARG | A | 156 | −94.600 | 44.778 | 34.222 | 1.00 | 17.98 | N |
| ATOM | 469 | NH2 | ARG | A | 156 | −94.577 | 44.478 | 31.944 | 1.00 | 17.82 | N |
| ATOM | 470 | C | ARG | A | 156 | −96.297 | 51.069 | 32.902 | 1.00 | 17.20 | C |
| ATOM | 471 | O | ARG | A | 156 | −96.912 | 51.585 | 31.959 | 1.00 | 16.98 | O |
| ATOM | 472 | N | VAL | A | 157 | −95.062 | 51.420 | 33.256 | 1.00 | 16.61 | N |
| ATOM | 473 | CA | VAL | A | 157 | −94.319 | 52.481 | 32.585 | 1.00 | 16.09 | C |
| ATOM | 474 | CB | VAL | A | 157 | −93.945 | 53.629 | 33.546 | 1.00 | 16.10 | C |
| ATOM | 475 | CG1 | VAL | A | 157 | −93.511 | 54.860 | 32.759 | 1.00 | 16.10 | C |
| ATOM | 476 | CG2 | VAL | A | 157 | −95.105 | 53.960 | 34.474 | 1.00 | 16.17 | C |
| ATOM | 477 | C | VAL | A | 157 | −93.051 | 51.880 | 32.001 | 1.00 | 15.84 | C |
| ATOM | 478 | O | VAL | A | 157 | −92.157 | 51.485 | 32.744 | 1.00 | 15.96 | O |
| ATOM | 479 | N | ARG | A | 158 | −92.985 | 51.806 | 30.675 | 1.00 | 15.34 | N |
| ATOM | 480 | CA | ARG | A | 158 | −91.833 | 51.240 | 29.977 | 1.00 | 15.13 | C |
| ATOM | 481 | CB | ARG | A | 158 | −92.318 | 50.315 | 28.871 | 1.00 | 14.82 | C |
| ATOM | 482 | CG | ARG | A | 158 | −91.226 | 49.616 | 28.070 | 1.00 | 14.70 | C |
| ATOM | 483 | CD | ARG | A | 158 | −91.849 | 48.800 | 26.949 | 1.00 | 14.49 | C |
| ATOM | 484 | NE | ARG | A | 158 | −92.874 | 47.891 | 27.461 | 1.00 | 14.22 | N |
| ATOM | 485 | CZ | ARG | A | 158 | −92.646 | 46.693 | 28.006 | 1.00 | 14.21 | C |
| ATOM | 486 | NH1 | ARG | A | 158 | −91.414 | 46.193 | 28.117 | 1.00 | 14.29 | N |
| ATOM | 487 | NE2 | ARG | A | 158 | −93.672 | 45.973 | 28.447 | 1.00 | 13.99 | N |
| ATOM | 488 | C | ARG | A | 158 | −90.958 | 52.346 | 29.399 | 1.00 | 14.98 | C |
| ATOM | 489 | O | ARG | A | 158 | −91.476 | 53.331 | 28.873 | 1.00 | 15.02 | O |
| ATOM | 490 | N | ALA | A | 159 | −89.640 | 52.179 | 29.518 | 1.00 | 14.80 | N |
| ATOM | 491 | CA | ALA | A | 159 | −88.659 | 53.044 | 28.852 | 1.00 | 14.93 | C |
| ATOM | 492 | CB | ALA | A | 159 | −87.725 | 53.668 | 29.667 | 1.00 | 14.73 | C |
| ATOM | 493 | C | ALA | A | 159 | −87.868 | 52.211 | 27.845 | 1.00 | 15.10 | C |
| ATOM | 494 | O | ALA | A | 159 | −87.461 | 51.093 | 28.161 | 1.00 | 14.95 | O |
| ATOM | 495 | N | MET | A | 160 | −87.678 | 52.752 | 26.639 | 1.00 | 15.49 | N |
| ATOM | 496 | CA | MET | A | 160 | −86.920 | 52.092 | 25.571 | 1.00 | 16.16 | C |
| ATOM | 497 | CB | MET | A | 160 | −87.864 | 51.351 | 24.622 | 1.00 | 16.52 | C |
| ATOM | 498 | CG | MET | A | 160 | −87.180 | 50.691 | 23.426 | 1.00 | 16.98 | C |
| ATOM | 499 | SD | MET | A | 160 | −88.345 | 49.887 | 22.318 | 1.00 | 17.63 | S |
| ATOM | 500 | CE | MET | A | 160 | −68.866 | 46.503 | 23.323 | 1.00 | 17.54 | C |
| ATOM | 501 | C | MET | A | 160 | −86.107 | 53.111 | 24.775 | 1.00 | 16.42 | C |
| ATOM | 502 | O | MET | A | 160 | −86.617 | 54.181 | 24.424 | 1.00 | 16.59 | O |
| ATOM | 503 | N | ALA | A | 161 | −84.663 | 52.749 | 24.463 | 1.00 | 16.59 | N |
| ATOM | 504 | CA | ALA | A | 161 | −83.952 | 53.614 | 23.708 | 1.00 | 17.08 | C |
| ATOM | 505 | CB | ALA | A | 161 | −82.543 | 53.516 | 24.276 | 1.00 | 17.03 | C |
| ATOM | 506 | C | ALA | A | 161 | −83.941 | 53.233 | 22.231 | 1.00 | 17.57 | C |
| ATOM | 507 | O | ALA | A | 161 | −83.843 | 52.051 | 21.904 | 1.00 | 17.68 | O |
| ATOM | 508 | N | ILE | A | 162 | −64.054 | 54.232 | 21.354 | 1.00 | 18.27 | N |
| ATOM | 509 | CA | ILE | A | 162 | −83.751 | 54.078 | 19.924 | 1.00 | 18.78 | C |
| ATOM | 510 | CB | ILE | A | 162 | −85.014 | 54.197 | 19.029 | 1.00 | 18.53 | C |
| ATOM | 511 | CG1 | ILE | A | 162 | −85.579 | 55.631 | 18.992 | 1.00 | 18.49 | C |
| ATOM | 512 | CD1 | ILE | A | 162 | −86.505 | 55.870 | 17.816 | 1.00 | 18.56 | C |
| ATOM | 513 | CG2 | ILE | A | 162 | −86.076 | 53.205 | 19.465 | 1.00 | 18.49 | C |
| ATOM | 514 | C | ILE | A | 162 | −82.710 | 55.108 | 19.474 | 1.00 | 19.64 | C |
| ATOM | 515 | O | ILE | A | 162 | −82.552 | 56.157 | 20.103 | 1.00 | 19.43 | O |
| ATOM | 516 | N | TYR | A | 163 | −82.018 | 54.799 | 18.378 | 1.00 | 21.00 | N |
| ATOM | 517 | CA | TYR | A | 163 | −81.080 | 55.733 | 17.746 | 1.00 | 22.33 | C |
| ATOM | 518 | CB | TYR | A | 163 | −80.025 | 54.973 | 16.931 | 1.00 | 21.91 | C |
| ATOM | 519 | CG | TYR | A | 163 | −79.062 | 54.180 | 17.789 | 1.00 | 21.28 | C |
| ATOM | 520 | CD1 | TYR | A | 163 | −78.191 | 54.825 | 18.668 | 1.00 | 21.45 | C |
| ATOM | 521 | CE1 | TYR | A | 163 | −77.309 | 54.108 | 19.462 | 1.00 | 21.53 | C |
| ATOM | 522 | CZ | TYR | A | 163 | −77.281 | 52.724 | 19.376 | 1.00 | 21.36 | C |
| ATOM | 523 | OH | TYR | A | 163 | −76.410 | 52.008 | 20.156 | 1.00 | 21.56 | O |
| ATOM | 524 | CE2 | TYR | A | 163 | −78.128 | 52.060 | 18.506 | 1.00 | 21.21 | C |
| ATOM | 525 | CD2 | TYR | A | 163 | −79.012 | 52.787 | 17.720 | 1.00 | 21.21 | C |
| ATOM | 526 | C | TYR | A | 163 | −81.826 | 56.739 | 16.857 | 1.00 | 23.97 | C |
| ATOM | 527 | O | TYR | A | 163 | −82.626 | 56.353 | 16.004 | 1.00 | 24.14 | O |
| ATOM | 528 | N | LYS | A | 164 | −81.537 | 58.024 | 17.065 | 1.00 | 26.10 | N |
| ATOM | 529 | CA | LYS | A | 164 | −82.220 | 59.135 | 16.385 | 1.00 | 27.86 | C |
| ATOM | 530 | CB | LYS | A | 164 | −81.710 | 60.455 | 16.974 | 1.00 | 28.77 | C |
| ATOM | 531 | CG | LYS | A | 164 | −82.482 | 61.713 | 16.631 | 1.00 | 29.61 | C |
| ATOM | 532 | CD | LYS | A | 164 | −81.822 | 62.889 | 17.338 | 1.00 | 29.96 | C |
| ATOM | 533 | CE | LYS | A | 164 | −82.416 | 64.225 | 16.925 | 1.00 | 30.68 | C |
| ATOM | 534 | NZ | LYS | A | 164 | −81.597 | 65.372 | 17.411 | 1.00 | 30.89 | N |
| ATOM | 535 | C | LYS | A | 164 | −82.016 | 59.114 | 14.862 | 1.00 | 28.86 | C |
| ATOM | 536 | O | LYS | A | 164 | −82.982 | 59.239 | 14.103 | 1.00 | 29.38 | O |
| ATOM | 537 | N | GLN | A | 165 | −80.763 | 58.943 | 14.434 | 1.00 | 29.06 | N |
| ATOM | 538 | CA | GLN | A | 165 | −80.399 | 58.911 | 13.005 | 1.00 | 29.89 | C |
| ATOM | 539 | CB | GLN | A | 165 | −78.874 | 58.880 | 12.835 | 1.00 | 29.89 | C |
| ATOM | 540 | CG | GLN | A | 165 | −78.179 | 60.204 | 13.117 | 1.00 | 30.40 | C |
| ATOM | 541 | CD | GLN | A | 165 | −76.663 | 60.093 | 13.173 | 1.00 | 30.68 | C |
| ATOM | 542 | OE1 | GLN | A | 165 | −76.083 | 59.066 | 12.820 | 1.00 | 30.65 | O |
| ATOM | 543 | NE2 | GLN | A | 165 | −76.012 | 61.160 | 13.628 | 1.00 | 31.04 | N |
| ATOM | 544 | C | GLN | A | 165 | −81.007 | 57.709 | 12.274 | 1.00 | 30.10 | C |
| ATOM | 545 | O | GLN | A | 165 | −80.913 | 56.579 | 12.758 | 1.00 | 30.12 | O |
| ATOM | 546 | N | SER | A | 166 | −81.598 | 57.961 | 11.102 | 1.00 | 30.14 | N |
| ATOM | 547 | CA | SER | A | 166 | −82.323 | 56.925 | 10.340 | 1.00 | 29.92 | C |
| ATOM | 548 | CB | SER | A | 166 | −83.168 | 57.546 | 9.215 | 1.00 | 30.15 | C |
| ATOM | 549 | OG | SER | A | 166 | −82.480 | 58.587 | 8.546 | 1.00 | 30.39 | O |
| ATOM | 550 | C | SER | A | 166 | −81.440 | 55.785 | 9.792 | 1.00 | 29.63 | C |
| ATOM | 551 | O | SER | A | 166 | −81.941 | 54.687 | 9.543 | 1.00 | 29.09 | O |
| ATOM | 552 | N | GLN | A | 167 | −80.144 | 56.049 | 9.618 | 1.00 | 29.23 | N |
| ATOM | 553 | CA | GLN | A | 167 | −79.181 | 55.024 | 9.170 | 1.00 | 29.16 | C |
| ATOM | 554 | CB | GLN | A | 167 | −77.947 | 55.662 | 8.507 | 1.00 | 29.27 | C |
| ATOM | 555 | CG | GLN | A | 167 | −77.014 | 56.469 | 9.413 | 1.00 | 29.58 | C |
| ATOM | 556 | CD | GLN | A | 167 | −77.426 | 57.923 | 9.584 | 1.00 | 30.21 | C |
| ATOM | 557 | OE1 | GLN | A | 167 | −76.549 | 58.314 | 9.248 | 1.00 | 31.03 | O |
| ATOM | 558 | NE2 | GLN | A | 167 | −76.525 | 58.729 | 10.134 | 1.00 | 29.60 | N |
| ATOM | 559 | C | GLN | A | 167 | −78.733 | 54.034 | 10.257 | 1.00 | 28.69 | C |
| ATOM | 560 | O | GLN | A | 167 | −78.085 | 53.036 | 9.936 | 1.00 | 29.01 | O |
| ATOM | 561 | N | HIS | A | 168 | −79.034 | 54.327 | 11.524 | 1.00 | 27.88 | N |
| ATOM | 562 | CA | HIS | A | 168 | −78.836 | 53.375 | 12.631 | 1.00 | 27.65 | C |
| ATOM | 563 | CB | HIS | A | 168 | −77.810 | 53.920 | 13.624 | 1.00 | 27.44 | C |
| ATOM | 564 | CG | HIS | A | 168 | −76.553 | 54.403 | 12.980 | 1.00 | 27.19 | C |
| ATOM | 565 | ND1 | HIS | A | 168 | −75.647 | 53.553 | 12.385 | 1.00 | 26.72 | N |
| ATOM | 566 | CE1 | HIS | A | 168 | −74.645 | 54.260 | 11.897 | 1.00 | 26.84 | C |
| ATOM | 567 | NE2 | HIS | A | 168 | −74.867 | 55.535 | 12.154 | 1.00 | 27.32 | N |
| ATOM | 568 | CD2 | HIS | A | 168 | −76.057 | 55.651 | 12.836 | 1.00 | 27.08 | C |
| ATOM | 569 | C | HIS | A | 168 | −80.144 | 53.087 | 13.362 | 1.00 | 27.96 | C |
| ATOM | 570 | O | HIS | A | 168 | −80.135 | 52.512 | 14.457 | 1.00 | 27.27 | O |
| ATOM | 571 | N | MET | A | 169 | −81.261 | 53.444 | 12.730 | 1.00 | 27.86 | N |
| ATOM | 572 | CA | MET | A | 169 | −82.575 | 53.421 | 13.362 | 1.00 | 28.04 | C |
| ATOM | 573 | CB | MET | A | 169 | −83.569 | 54.213 | 12.503 | 1.00 | 29.38 | C |
| ATOM | 574 | CG | MET | A | 169 | −84.968 | 54.356 | 13.076 | 1.00 | 30.82 | C |
| ATOM | 575 | SD | MET | A | 169 | −85.817 | 55.870 | 12.567 | 1.00 | 32.31 | S |
| ATOM | 576 | CE | MET | A | 169 | −85.457 | 56.951 | 13.949 | 1.00 | 31.81 | C |
| ATOM | 577 | C | MET | A | 169 | −83.092 | 52.004 | 13.635 | 1.00 | 26.96 | C |
| ATOM | 578 | O | MET | A | 169 | −83.896 | 51.797 | 14.549 | 1.00 | 26.40 | O |
| ATOM | 579 | N | THR | A | 170 | −82.626 | 54.040 | 12.841 | 1.00 | 25.54 | N |
| ATOM | 580 | CA | THR | A | 170 | −82.956 | 49.627 | 13.030 | 1.00 | 24.52 | C |
| ATOM | 581 | CB | THR | A | 170 | −82.894 | 48.862 | 11.691 | 1.00 | 24.51 | C |
| ATOM | 582 | OG1 | THR | A | 170 | −81.646 | 49.129 | 11.037 | 1.00 | 24.55 | O |
| ATOM | 583 | CG1 | THR | A | 170 | −84.042 | 49.279 | 10.783 | 1.00 | 24.39 | C |
| ATOM | 584 | C | THR | A | 170 | −82.056 | 48.898 | 14.038 | 1.00 | 23.17 | C |
| ATOM | 585 | O | THR | A | 170 | −82.344 | 47.750 | 14.382 | 1.00 | 23.82 | O |
| ATOM | 586 | N | GLU | A | 171 | −80.982 | 49.541 | 14.502 | 1.00 | 21.79 | N |
| ATOM | 587 | CA | GLU | A | 171 | −80.054 | 48.920 | 15.459 | 1.00 | 20.80 | C |
| ATOM | 588 | CB | GLU | A | 171 | −78.709 | 49.652 | 15.482 | 1.00 | 20.90 | C |
| ATOM | 589 | CG | GLU | A | 171 | −77.987 | 49.673 | 14.148 | 1.00 | 21.15 | C |
| ATOM | 590 | CD | GLU | A | 171 | −76.698 | 50.469 | 14.159 | 1.00 | 21.23 | C |
| ATOM | 591 | OE1 | GLU | A | 171 | −76.120 | 50.650 | 13.066 | 1.00 | 21.76 | N |
| ATOM | 592 | OE2 | GLU | A | 171 | −76.236 | 50.902 | 15.236 | 1.00 | 21.38 | O |
| ATOM | 593 | C | GLU | A | 171 | −80.625 | 48.920 | 16.871 | 1.00 | 19.91 | C |
| ATOM | 594 | O | GLU | A | 171 | −81.256 | 49.890 | 17.289 | 1.00 | 18.81 | O |
| ATOM | 595 | N | VAL | A | 172 | −80.376 | 47.838 | 17.609 | 1.00 | 18.94 | N |
| ATOM | 596 | CA | VAL | A | 172 | −80.751 | 47.762 | 19.014 | 1.00 | 18.73 | C |
| ATOM | 597 | CB | VAL | A | 172 | −80.689 | 46.311 | 19.558 | 1.00 | 18.50 | C |
| ATOM | 598 | CG1 | VAL | A | 172 | −80.975 | 46.270 | 21.057 | 1.00 | 18.40 | C |
| ATOM | 599 | CG2 | VAL | A | 172 | −81.684 | 45.425 | 18.814 | 1.00 | 18.57 | C |
| ATOM | 600 | C | VAL | A | 172 | −79.795 | 48.671 | 19.786 | 1.00 | 18.63 | C |
| ATOM | 601 | O | VAL | A | 172 | −78.576 | 48.568 | 19.623 | 1.00 | 18.49 | O |
| ATOM | 602 | N | VAL | A | 173 | −80.347 | 49.564 | 20.608 | 1.00 | 18.31 | N |
| ATOM | 603 | CA | VAL | A | 173 | −79.529 | 50.380 | 21.499 | 1.00 | 18.21 | C |
| ATOM | 604 | CB | VAL | A | 173 | −80.272 | 51.613 | 22.063 | 1.00 | 18.41 | C |
| ATOM | 605 | CG1 | VAL | A | 173 | −79.339 | 52.448 | 22.939 | 1.00 | 18.46 | C |
| ATOM | 606 | CG2 | VAL | A | 173 | −80.833 | 52.472 | 20.940 | 1.00 | 18.55 | C |
| ATOM | 607 | C | VAL | A | 173 | −79.115 | 49.466 | 22.649 | 1.00 | 18.05 | C |
| ATOM | 608 | O | VAL | A | 173 | −79.966 | 48.942 | 23.375 | 1.00 | 17.43 | O |
| ATOM | 609 | N | ARG | A | 174 | −77.810 | 49.267 | 22.788 | 1.00 | 17.95 | N |
| ATOM | 610 | CA | ARG | A | 174 | −77.255 | 48.448 | 23.854 | 1.00 | 18.51 | C |
| ATOM | 611 | CB | ARG | A | 174 | −76.993 | 47.006 | 23.366 | 1.00 | 18.81 | C |
| ATOM | 612 | CG | ARG | A | 174 | −76.112 | 46.884 | 22.129 | 1.00 | 19.15 | C |
| ATOM | 613 | CD | ARG | A | 174 | −75.911 | 45.430 | 21.699 | 1.00 | 19.79 | C |
| ATOM | 614 | NE | ARG | A | 174 | −74.892 | 44.731 | 22.491 | 1.00 | 20.17 | N |
| ATOM | 615 | CZ | ARG | A | 174 | −74.369 | 43.534 | 22.195 | 1.00 | 20.88 | C |
| ATOM | 616 | NH1 | ARG | A | 174 | −74.745 | 42.861 | 21.106 | 1.00 | 21.04 | N |
| ATOM | 617 | NH2 | ARG | A | 174 | −73.446 | 42.998 | 22.996 | 1.00 | 21.01 | N |
| ATOM | 618 | C | ARG | A | 174 | −75.974 | 49.097 | 24.344 | 1.00 | 18.67 | C |
| ATOM | 619 | O | ARG | A | 174 | −75.470 | 50.051 | 23.740 | 1.00 | 18.14 | O |
| ATOM | 620 | N | ARG | A | 175 | −75.455 | 48.574 | 25.444 | 1.00 | 19.20 | N |
| ATOM | 621 | CA | ARG | A | 175 | −74.187 | 49.052 | 25.983 | 1.00 | 19.94 | C |
| ATOM | 622 | CB | ARG | A | 175 | −73.970 | 48.521 | 27.395 | 1.00 | 20.13 | C |
| ATOM | 623 | CG | ARG | A | 175 | −75.008 | 49.053 | 28.374 | 1.00 | 20.11 | C |
| ATOM | 624 | CD | ARG | A | 175 | −74.409 | 49.354 | 29.729 | 1.00 | 20.30 | O |
| ATOM | 625 | NE | ARG | A | 175 | −74.173 | 48.146 | 30.501 | 1.00 | 20.44 | N |
| ATOM | 626 | CZ | ARG | A | 175 | −73.637 | 48.111 | 31.720 | 1.00 | 20.54 | C |
| ATOM | 627 | NH1 | ARG | A | 175 | −73.474 | 46.942 | 32.331 | 1.00 | 20.56 | N |
| ATOM | 628 | NH2 | ARG | A | 175 | −73.242 | 49.225 | 32.326 | 1.00 | 20.88 | N |
| ATOM | 629 | C | ARG | A | 175 | −73.026 | 48.675 | 25.073 | 1.00 | 20.42 | C |
| ATOM | 630 | O | ARG | A | 175 | −73.082 | 47.683 | 24.342 | 1.00 | 20.53 | O |
| ATOM | 631 | N | CYS | A | 176 | −71.975 | 49.484 | 25.124 | 1.00 | 21.38 | N |
| ATOM | 632 | CA | CYS | A | 176 | −70.783 | 49.264 | 24.316 | 1.00 | 21.50 | C |
| ATOM | 633 | CB | CYS | A | 176 | −70.000 | 50.568 | 24.220 | 1.00 | 21.73 | C |
| ATOM | 634 | SG | CYS | A | 176 | −69.225 | 51.090 | 25.768 | 1.00 | 22.09 | S |
| ATOM | 635 | C | CYS | A | 176 | −69.950 | 48.148 | 24.961 | 1.00 | 21.42 | C |
| ATOM | 636 | O | CYS | A | 176 | −70.250 | 47.741 | 26.084 | 1.00 | 21.21 | O |
| ATOM | 637 | N | PRO | A | 177 | −68.918 | 47.633 | 24.258 | 1.00 | 21.95 | N |
| ATOM | 638 | CA | PRO | A | 177 | −68.125 | 46.526 | 24.826 | 1.00 | 21.97 | C |
| ATOM | 639 | CB | PRO | A | 177 | −67.083 | 46.246 | 23.736 | 1.00 | 21.87 | C |
| ATOM | 640 | CG | PRO | A | 177 | −67.746 | 46.678 | 22.477 | 1.00 | 22.03 | C |
| ATOM | 641 | CD | PRO | A | 177 | −68.545 | 47.892 | 22.852 | 1.00 | 21.93 | C |
| ATOM | 642 | C | PRO | A | 177 | −67.445 | 46.807 | 26.176 | 1.00 | 22.09 | C |
| ATOM | 643 | O | PRO | A | 177 | −67.330 | 45.892 | 26.995 | 1.00 | 22.32 | O |
| ATOM | 644 | N | HIS | A | 178 | −66.994 | 48.043 | 26.399 | 1.00 | 22.32 | N |
| ATOM | 645 | CA | HIS | A | 178 | −66.481 | 48.462 | 27.720 | 1.00 | 22.41 | C |
| ATOM | 646 | CB | HIS | A | 178 | −65.816 | 49.859 | 27.652 | 1.00 | 22.62 | C |
| ATOM | 647 | CG | HIS | A | 178 | −65.591 | 50.502 | 28.991 | 1.00 | 22.54 | C |
| ATOM | 648 | ND1 | HIS | A | 178 | −64.996 | 49.842 | 30.045 | 1.00 | 22.42 | N |
| ATOM | 649 | CES | HIS | A | 178 | −64.934 | 50.649 | 31.090 | 1.00 | 22.44 | C |
| ATOM | 650 | NE2 | HIS | A | 178 | −65.458 | 51.813 | 30.749 | 1.00 | 22.50 | N |
| ATOM | 651 | CD2 | HIS | A | 178 | −65.673 | 51.749 | 29.442 | 1.00 | 22.46 | C |
| ATOM | 652 | C | HIS | A | 178 | −67.567 | 48.424 | 28.807 | 1.00 | 22.73 | C |
| ATOM | 653 | O | HIS | A | 178 | −67.410 | 47.739 | 29.824 | 1.00 | 23.18 | O |
| ATOM | 654 | N | HIS | A | 179 | −66.664 | 49.143 | 28.587 | 1.00 | 22.43 | N |
| ATOM | 655 | CA | HIS | A | 179 | −69.695 | 49.289 | 29.621 | 1.00 | 22.50 | C |
| ATOM | 656 | CB | HIS | A | 179 | −70.600 | 50.498 | 29.342 | 1.00 | 22.13 | C |
| ATOM | 657 | CG | HIS | A | 179 | −69.978 | 51.805 | 29.726 | 1.00 | 21.78 | C |
| ATOM | 658 | ND1 | HIS | A | 179 | −69.837 | 52.857 | 28.847 | 1.00 | 21.85 | N |
| ATOM | 659 | CE1 | HIS | A | 179 | −69.249 | 53.866 | 29.466 | 1.00 | 21.54 | C |
| ATOM | 660 | NE2 | HIS | A | 179 | −66.999 | 53.505 | 30.710 | 1.00 | 21.22 | N |
| ATOM | 661 | CD2 | HIS | A | 179 | −69.444 | 52.221 | 30.899 | 1.00 | 21.44 | C |
| ATOM | 662 | C | HIS | A | 179 | −70.506 | 48.015 | 29.891 | 1.00 | 23.20 | C |
| ATOM | 663 | O | HIS | A | 179 | −70.879 | 47.773 | 31.038 | 1.00 | 22.97 | C |
| ATOM | 664 | N | GLU | A | 860 | −70.736 | 47.187 | 28.870 | 1.00 | 24.27 | N |
| ATOM | 665 | CA | GLU | A | 180 | −71.340 | 45.854 | 29.075 | 1.00 | 25.38 | C |
| ATOM | 666 | CB | GLU | A | 180 | −71.599 | 45.144 | 27.738 | 1.00 | 25.54 | C |
| ATOM | 667 | CG | GLU | A | 180 | −72.483 | 43.903 | 27.858 | 1.00 | 25.99 | C |
| ATOM | 668 | CO | GLU | A | 180 | −72.420 | 43.000 | 26.644 | 1.00 | 26.00 | C |
| ATOM | 669 | OE1 | GLU | A | 180 | −72.553 | 43.506 | 25.512 | 1.00 | 26.11 | O |
| ATOM | 670 | OE2 | GLU | A | 180 | −72.249 | 41.776 | 26.825 | 1.00 | 26.36 | O |
| ATOM | 671 | C | GLU | A | 180 | −70.481 | 44.944 | 29.970 | 1.00 | 26.60 | C |
| ATOM | 672 | O | GLU | A | 180 | −71.018 | 44.116 | 30.707 | 1.00 | 26.55 | O |
| ATOM | 673 | N | ARG | A | 181 | −69.158 | 45.108 | 29.902 | 1.00 | 28.13 | N |
| ATOM | 674 | CA | ARG | A | 181 | −68.211 | 44.313 | 30.701 | 1.00 | 29.67 | C |
| ATOM | 675 | CB | ARG | A | 181 | −66.919 | 44.073 | 29.894 | 1.00 | 30.54 | C |
| ATOM | 676 | CG | ARG | A | 181 | −67.009 | 42.925 | 28.897 | 1.00 | 31.84 | C |
| ATOM | 677 | CD | ARG | A | 181 | −66.974 | 41.570 | 29.593 | 1.00 | 32.76 | C |
| ATOM | 678 | NE | ARG | A | 181 | −66.517 | 40.505 | 28.700 | 1.00 | 33.86 | N |
| ATOM | 679 | CZ | ARG | A | 181 | −67.257 | 39.892 | 27.772 | 1.00 | 34.70 | C |
| ATOM | 680 | NH1 | ARG | A | 181 | −68.537 | 40.215 | 27.569 | 1.00 | 35.19 | N |
| ATOM | 681 | NH2 | ARG | A | 181 | −66.706 | 36.934 | 27.029 | 1.00 | 34.90 | N |
| ATOM | 682 | C | ARG | A | 181 | −67.871 | 44.913 | 32.079 | 1.00 | 29.78 | C |
| ATOM | 683 | O | ARG | A | 181 | −66.977 | 44.408 | 32.764 | 1.00 | 29.61 | O |
| ATOM | 664 | N | CYS | A | 182 | −66.580 | 45.964 | 32.495 | 1.00 | 30.48 | N |
| ATOM | 665 | CA | CYS | A | 182 | −66.338 | 46.592 | 33.797 | 1.00 | 30.66 | C |
| ATOM | 686 | CB | CYS | A | 182 | −68.908 | 48.018 | 33.849 | 1.00 | 31.12 | C |
| ATOM | 687 | SG | CYS | A | 182 | −67.842 | 49.271 | 33.103 | 1.00 | 31.92 | S |
| ATOM | 688 | C | CYS | A | 182 | −68.925 | 45.770 | 34.942 | 1.00 | 30.87 | C |
| ATOM | 689 | O | CYS | A | 182 | −69.954 | 45.103 | 34.776 | 1.00 | 31.57 | O |
| ATOM | 690 | N | SER | A | 183 | −68.270 | 45.843 | 36.101 | 1.00 | 30.13 | N |
| ATOM | 691 | CA | SER | A | 183 | −68.771 | 42.249 | 37.344 | 1.00 | 29.92 | C |
| ATOM | 692 | CB | SER | A | 183 | −67.608 | 44.758 | 38.213 | 1.00 | 29.70 | C |
| ATOM | 693 | OG | SER | A | 183 | −66.894 | 43.730 | 37.554 | 1.00 | 29.48 | O |
| ATOM | 694 | C | SER | A | 183 | −69.626 | 46.274 | 38.093 | 1.00 | 29.65 | C |
| ATOM | 695 | O | SER | A | 183 | −69.298 | 46.683 | 39.209 | 1.00 | 29.92 | O |
| ATOM | 696 | N | ASP | A | 184 | −70.725 | 46.677 | 37.454 | 1.00 | 29.01 | N |
| ATOM | 697 | CA | ASP | A | 184 | −71.682 | 47.639 | 38.009 | 1.00 | 28.13 | C |
| ATOM | 698 | CB | ASP | A | 184 | −71.695 | 48.937 | 37.177 | 1.00 | 28.06 | C |
| ATOM | 699 | CG | ASP | A | 184 | −72.109 | 48.722 | 35.707 | 1.00 | 28.19 | C |
| ATOM | 700 | OD1 | ASP | A | 184 | −72.537 | 47.606 | 35.325 | 1.00 | 27.47 | O |
| ATOM | 701 | OD2 | ASP | A | 184 | −72.001 | 49.690 | 34.922 | 1.00 | 27.68 | O |
| ATOM | 702 | C | ASP | A | 184 | −73.075 | 47.002 | 38.079 | 1.00 | 27.71 | C |
| ATOM | 703 | O | ASP | A | 184 | −74.093 | 47.701 | 38.018 | 1.00 | 27.86 | O |
| ATOM | 704 | N | SER | A | 185 | −73.108 | 45.674 | 38.205 | 1.00 | 26.76 | N |
| ATOM | 705 | CA | SER | A | 185 | −74.356 | 44.922 | 38.190 | 1.00 | 26.46 | C |
| ATOM | 706 | CB | SER | A | 185 | −74.077 | 43.419 | 38.092 | 1.00 | 26.56 | C |
| ATOM | 707 | OG | SER | A | 185 | −75.282 | 42.673 | 38.044 | 1.00 | 26.66 | O |
| ATOM | 708 | C | SER | A | 185 | −75.144 | 45.216 | 39.460 | 1.00 | 25.62 | C |
| ATOM | 709 | O | SER | A | 185 | −74.568 | 45.278 | 40.546 | 1.00 | 25.80 | O |
| ATOM | 710 | N | ASP | A | 186 | −76.453 | 45.408 | 39.308 | 1.00 | 24.74 | N |
| ATOM | 711 | CA | ASP | A | 186 | −77.355 | 45.596 | 40.446 | 1.00 | 24.10 | C |
| ATOM | 712 | CB | ASP | A | 186 | −76.253 | 46.835 | 40.229 | 1.00 | 23.75 | C |
| ATOM | 713 | CG | ASP | A | 186 | −79.330 | 46.637 | 39.157 | 1.00 | 23.41 | C |
| ATOM | 714 | OD1 | ASP | A | 186 | −79.339 | 45.612 | 38.446 | 1.00 | 23.02 | O |
| ATOM | 715 | OD2 | ASP | A | 186 | −80.177 | 47.542 | 39.023 | 1.00 | 23.41 | O |
| ATOM | 716 | C | ASP | A | 186 | −78.185 | 44.341 | 40.767 | 1.00 | 23.70 | C |
| ATOM | 717 | O | ASP | A | 186 | −79.120 | 44.408 | 41.567 | 1.00 | 23.58 | O |
| ATOM | 718 | N | GLY | A | 187 | −77.857 | 43.214 | 40.129 | 1.00 | 23.05 | N |
| ATOM | 719 | CA | GLY | A | 187 | −78.567 | 41.952 | 40.338 | 1.00 | 23.08 | C |
| ATOM | 720 | C | GLY | A | 187 | −79.892 | 41.783 | 39.606 | 1.00 | 22.50 | C |
| ATOM | 721 | O | GLY | A | 187 | −80.454 | 40.690 | 39.619 | 1.00 | 23.19 | O |
| ATOM | 722 | N | LEU | A | 188 | −80.395 | 42.846 | 38.976 | 1.00 | 22.14 | N |
| ATOM | 723 | CA | LEU | A | 188 | −81.685 | 42.829 | 38.270 | 1.00 | 21.61 | C |
| ATOM | 724 | CB | LEU | A | 188 | −82.634 | 43.866 | 38.892 | 1.00 | 21.79 | C |
| ATOM | 725 | CG | LEU | A | 188 | −82.820 | 43.817 | 40.416 | 1.00 | 22.14 | C |
| ATOM | 726 | CD1 | LEU | A | 188 | −83.805 | 44.878 | 40.888 | 1.00 | 22.39 | C |
| ATOM | 727 | CD2 | LEU | A | 188 | −83.278 | 42.442 | 40.879 | 1.00 | 22.25 | C |
| ATOM | 728 | C | LEU | A | 188 | −81.514 | 43.092 | 36.766 | 1.00 | 20.99 | C |
| ATOM | 729 | O | LEU | A | 188 | −82.023 | 42.330 | 35.936 | 1.00 | 21.46 | O |
| ATOM | 730 | N | ALA | A | 189 | −80.789 | 44.156 | 36.423 | 1.00 | 20.06 | N |
| ATOM | 731 | CA | ALA | A | 189 | −80.599 | 44.575 | 35.033 | 1.00 | 19.56 | C |
| ATOM | 732 | CO | ALA | A | 189 | −80.055 | 45.998 | 34.986 | 1.00 | 19.46 | C |
| ATOM | 733 | C | ALA | A | 189 | −79.653 | 43.645 | 34.268 | 1.00 | 19.20 | C |
| ATOM | 734 | O | ALA | A | 189 | −78.565 | 43.335 | 34.770 | 1.00 | 18.56 | O |
| ATOM | 735 | N | PRO | A | 190 | −80.051 | 43.210 | 33.050 | 1.00 | 18.89 | N |
| ATOM | 736 | CA | PRO | A | 190 | −79.090 | 42.526 | 32.180 | 1.00 | 18.97 | C |
| ATOM | 737 | CO | PRO | A | 190 | −79.914 | 42.165 | 30.936 | 1.00 | 19.09 | C |
| ATOM | 738 | CG | PRO | A | 190 | −81.341 | 42.255 | 31.352 | 1.00 | 18.84 | C |
| ATOM | 739 | CD | PRO | A | 190 | −81.394 | 43.283 | 32.437 | 1.00 | 19.04 | C |
| ATOM | 740 | C | PRO | A | 190 | −77.931 | 43.462 | 31.806 | 1.00 | 18.90 | C |
| ATOM | 741 | O | PRO | A | 190 | −78.166 | 44.657 | 31.596 | 1.00 | 18.50 | O |
| ATOM | 742 | N | PRO | A | 191 | −76.693 | 42.932 | 31.730 | 1.00 | 19.04 | N |
| ATOM | 743 | CA | PRO | A | 191 | −75.522 | 43.790 | 31.496 | 1.00 | 19.27 | C |
| ATOM | 744 | CO | PRO | A | 191 | −74.336 | 42.816 | 31.607 | 1.00 | 19.14 | C |
| ATOM | 745 | CG | PRO | A | 191 | −74.912 | 41.471 | 31.360 | 1.00 | 19.22 | C |
| ATOM | 746 | CD | PRO | A | 191 | −76.314 | 41.514 | 31.875 | 1.00 | 19.04 | C |
| ATCN | 747 | C | PRO | A | 191 | −75.504 | 44.549 | 30.153 | 1.00 | 19.02 | C |
| ATOM | 748 | O | PRO | A | 191 | −74.877 | 45.606 | 30.075 | 1.00 | 19.34 | O |
| ATOM | 749 | N | GLN | A | 192 | −76.182 | 44.032 | 29.126 | 1.00 | 19.10 | N |
| ATOM | 750 | CA | GLN | A | 192 | −76.326 | 44.742 | 27.842 | 1.00 | 19.03 | C |
| ATOM | 751 | CB | GLN | A | 192 | −76.861 | 43.819 | 26.736 | 1.00 | 19.89 | C |
| ATOM | 752 | CG | GLN | A | 192 | −75.923 | 42.735 | 26.248 | 1.00 | 20.69 | C |
| ATOM | 753 | CD | GLN | A | 192 | −76.544 | 41.875 | 25.158 | 1.00 | 21.63 | C |
| ATOM | 754 | OE1 | GLN | A | 192 | −77.495 | 42.281 | 24.486 | 1.00 | 23.23 | O |
| ATOM | 755 | NE2 | GLN | A | 192 | −76.001 | 40.676 | 24.975 | 1.00 | 22.81 | N |
| ATOM | 756 | C | GLN | A | 192 | −77.253 | 45.963 | 27.889 | 1.00 | 18.49 | C |
| ATOM | 757 | O | GLN | A | 192 | −77.207 | 46.782 | 26.973 | 1.00 | 18.80 | O |
| ATOM | 758 | N | HIS | A | 193 | −78.111 | 46.073 | 28.909 | 1.00 | 17.79 | N |
| ATOM | 759 | CA | HIS | A | 193 | −79.138 | 47.128 | 29.935 | 1.00 | 16.99 | C |
| ATOM | 760 | CB | HIS | A | 193 | −80.724 | 46.780 | 29.910 | 1.00 | 16.80 | C |
| ATOM | 761 | CG | HIS | A | 193 | −81.259 | 45.795 | 29.364 | 1.00 | 16.42 | C |
| ATOM | 762 | ND1 | HIS | A | 193 | −82.618 | 45.916 | 29.561 | 1.00 | 16.22 | N |
| ATOM | 763 | CE1 | HIS | A | 193 | −83.238 | 44.912 | 28.968 | 1.00 | 15.89 | C |
| ATOM | 764 | NE2 | HIS | A | 193 | −82.332 | 44.151 | 28.382 | 1.00 | 15.87 | N |
| ATOM | 765 | CD2 | HIS | A | 193 | −81.086 | 44.678 | 28.617 | 1.00 | 16.20 | C |
| ATOM | 766 | C | HIS | A | 193 | −78.568 | 48.510 | 29.260 | 1.00 | 16.73 | C |
| ATOM | 767 | O | HIS | A | 193 | −77.819 | 48.682 | 30.226 | 1.00 | 16.04 | O |
| ATOM | 768 | N | HIS | A | 194 | −78.943 | 49.493 | 28.44 | 1.00 | 16.92 | N |
| ATOM | 769 | CA | LEU | A | 194 | −78.496 | 50.874 | 28.625 | 1.00 | 16.75 | C |
| ATOM | 770 | CB | LEU | A | 194 | −78.644 | 51.670 | 27.317 | 1.00 | 17.06 | C |
| ATOM | 771 | CG | LEU | A | 194 | −78.518 | 53.199 | 27.370 | 1.00 | 17.32 | C |
| ATOM | 772 | CD1 | LEU | A | 194 | −77.192 | 53.637 | 27.963 | 1.00 | 17.50 | C |
| ATOM | 773 | CD2 | LEU | A | 194 | −78.699 | 53.796 | 25.985 | 1.00 | 17.66 | C |
| ATOM | 774 | C | LEU | A | 194 | −79.254 | 51.574 | 29.748 | 1.00 | 16.68 | C |
| ATOM | 775 | O | LEU | A | 194 | −78.640 | 52.237 | 30.588 | 1.00 | 16.14 | O |
| ATOM | 776 | N | ILE | A | 195 | −80.584 | 51.447 | 29.731 | 1.00 | 16.40 | N |
| ATOM | 777 | CA | ILE | A | 195 | −83.462 | 52.202 | 30.707 | 1.00 | 16.53 | C |
| ATOM | 778 | CB | ILE | A | 195 | −82.793 | 52.587 | 30.056 | 1.00 | 16.53 | C |
| ATOM | 779 | CG1 | ILE | A | 195 | −82.503 | 53.459 | 28.820 | 1.00 | 16.58 | C |
| ATOM | 780 | CD1 | ILE | A | 195 | −83.730 | 53.982 | 28.096 | 1.00 | 16.66 | C |
| ATOM | 781 | CG2 | ILE | A | 195 | −83.644 | 53.369 | 31.062 | 1.00 | 16.42 | C |
| ATOM | 782 | C | ILE | A | 195 | −81.774 | 51.134 | 31.854 | 1.00 | 16.45 | C |
| ATOM | 783 | O | ILE | A | 195 | −82.135 | 49.979 | 31.621 | 1.00 | 15.92 | O |
| ATOM | 784 | N | ARG | A | 196 | −81.626 | 51.629 | 33.083 | 1.00 | 16.57 | N |
| ATOM | 785 | CA | ARG | A | 196 | −82.074 | 50.946 | 34.296 | 1.00 | 16.95 | C |
| ATOM | 786 | CB | ARG | A | 196 | −80.885 | 50.563 | 35.173 | 1.00 | 16.90 | C |
| ATOM | 787 | CG | ARG | A | 196 | −79.894 | 49.596 | 34.562 | 1.00 | 17.12 | C |
| ATOM | 788 | CD | ARG | A | 196 | −78.775 | 49.360 | 35.564 | 1.00 | 17.13 | C |
| ATOM | 789 | NE | ARG | A | 196 | −77.700 | 48.526 | 35.035 | 1.00 | 17.54 | N |
| ATOM | 790 | CZ | ARG | A | 196 | −76.547 | 48.282 | 35.662 | 1.00 | 17.55 | C |
| ATOM | 791 | NH1 | ARG | A | 196 | −76.293 | 48.807 | 36.859 | 1.00 | 17.75 | N |
| ATOM | 792 | NH2 | ARG | A | 196 | −75.635 | 47.508 | 35.082 | 1.00 | 17.78 | N |
| ATOM | 793 | C | ARG | A | 196 | −82.950 | 51.880 | 35.115 | 1.00 | 17.01 | C |
| ATOM | 794 | O | ARG | A | 196 | −82.986 | 53.085 | 34.869 | 1.00 | 16.53 | O |
| ATOM | 795 | N | VAL | A | 197 | −83.632 | 51.301 | 36.100 | 1.00 | 17.61 | N |
| ATOM | 796 | CA | VAL | A | 197 | −84.335 | 52.050 | 37.136 | 1.00 | 18.37 | C |
| ATOM | 797 | CB | VAL | A | 197 | −85.841 | 51.722 | 37.144 | 1.00 | 18.32 | C |
| ATOM | 798 | CG1 | VAL | A | 197 | −86.534 | 52.291 | 38.382 | 1.00 | 18.41 | C |
| ATOM | 799 | CG2 | VAL | A | 197 | −86.492 | 52.247 | 35.873 | 1.00 | 18.32 | C |
| ATOM | 800 | C | VAL | A | 197 | −83.718 | 51.697 | 38.485 | 1.00 | 19.43 | C |
| ATOM | 801 | O | VAL | A | 197 | −83.497 | 50.518 | 38.780 | 1.00 | 19.61 | O |
| ATOM | 802 | N | GLU | A | 198 | −83.464 | 52.720 | 39.303 | 1.00 | 20.31 | N |
| ATOM | 803 | CA | GLU | A | 198 | −82.940 | 52.528 | 40.662 | 1.00 | 21.36 | C |
| ATOM | 804 | CB | GLU | A | 198 | −81.765 | 53.476 | 40.958 | 1.00 | 22.15 | C |
| ATOM | 805 | CG | GLU | A | 198 | −82.064 | 54.970 | 40.862 | 1.00 | 22.85 | C |
| ATOM | 806 | CD | GLU | A | 198 | −81.019 | 55.834 | 41.557 | 1.00 | 23.59 | C |
| ATOM | 807 | OE1 | GLU | A | 198 | −81.392 | 56.919 | 42.048 | 1.00 | 25.09 | O |
| ATOM | 808 | OE2 | GLU | A | 198 | −79.833 | 55.439 | 41.616 | 1.00 | 24.35 | O |
| ATOM | 809 | C | GLU | A | 198 | −84.037 | 52.710 | 41.705 | 1.00 | 21.49 | C |
| ATOM | 810 | O | GLU | A | 198 | −85.012 | 53.435 | 41.477 | 1.00 | 21.75 | O |
| ATOM | 811 | N | GLY | A | 199 | −83.867 | 52.041 | 42.842 | 1.00 | 21.41 | N |
| ATOM | 812 | CA | GLY | A | 199 | −84.748 | 52.204 | 43.994 | 1.00 | 21.44 | C |
| ATOM | 813 | C | GLY | A | 199 | −86.153 | 51.661 | 43.804 | 1.00 | 21.59 | C |
| ATOM | 814 | O | GLY | A | 199 | −87.114 | 52.259 | 44.290 | 1.00 | 21.61 | O |
| ATOM | 815 | N | ASN | A | 200 | −86.274 | 50.537 | 43.099 | 1.00 | 21.43 | N |
| ATOM | 816 | CA | ASN | A | 200 | −87.572 | 49.898 | 42.879 | 1.00 | 21.45 | C |
| ATOM | 817 | CB | ASN | A | 200 | −88.341 | 50.611 | 41.758 | 1.00 | 21.58 | C |
| ATOM | 818 | CG | ASN | A | 200 | −89.817 | 50.241 | 41.722 | 1.00 | 21.85 | C |
| ATOM | 819 | OD1 | ASN | A | 200 | −90.256 | 49.274 | 42.348 | 1.00 | 22.19 | O |
| ATOM | 820 | ND1 | ASN | A | 200 | −90.590 | 51.000 | 40.952 | 1.00 | 22.05 | N |
| ATOM | 821 | C | ASN | A | 200 | −87.401 | 48.402 | 42.581 | 1.00 | 21.43 | C |
| ATOM | 822 | O | ASN | A | 200 | −86.953 | 48.013 | 41.499 | 1.00 | 21.37 | O |
| ATOM | 823 | N | LEU | A | 201 | −87.791 | 47.579 | 43.554 | 1.00 | 21.20 | N |
| ATOM | 824 | CA | LEU | A | 201 | −87.580 | 46.131 | 43.520 | 1.00 | 21.01 | C |
| ATOM | 825 | CB | LEU | A | 201 | −87.660 | 45.579 | 44.953 | 1.00 | 21.89 | C |
| ATOM | 826 | CG | LEU | A | 201 | −87.014 | 44.241 | 45.316 | 1.00 | 22.49 | C |
| ATOM | 827 | CD1 | LEU | A | 201 | −85.533 | 44.188 | 44.965 | 1.00 | 22.85 | C |
| ATOM | 828 | CD2 | LEU | A | 201 | −87.213 | 43.986 | 46.805 | 1.00 | 22.72 | C |
| ATOM | 829 | C | LEU | A | 201 | −88.556 | 45.389 | 42.593 | 1.00 | 20.31 | C |
| ATOM | 830 | O | LEU | A | 201 | −88.345 | 44.212 | 42.285 | 1.00 | 20.72 | O |
| ATOM | 831 | N | ARG | A | 202 | −89.604 | 46.080 | 42.145 | 1.00 | 19.07 | N |
| ATOM | 832 | CA | ARG | A | 202 | −90.563 | 45.545 | 41.180 | 1.00 | 18.25 | C |
| ATOM | 833 | CB | ARG | A | 202 | −91.955 | 46.126 | 41.473 | 1.00 | 17.92 | C |
| ATOM | 834 | CG | ARG | A | 202 | −92.451 | 45.869 | 42.890 | 1.00 | 17.79 | C |
| ATOM | 835 | CD | ARG | A | 202 | −93.742 | 46.617 | 43.185 | 1.00 | 17.77 | C |
| ATOM | 836 | NE | ARG | A | 202 | −94.859 | 46.108 | 42.395 | 1.00 | 17.90 | N |
| ATOM | 837 | CZ | ARG | A | 202 | −96.061 | 46.680 | 42.290 | 1.00 | 18.05 | C |
| ATOM | 838 | NH1 | ARG | A | 202 | −96.348 | 47.820 | 42.921 | 1.00 | 18.27 | N |
| ATOM | 839 | NH2 | ARG | A | 202 | −96.987 | 46.106 | 41.523 | 1.00 | 18.12 | N |
| ATOM | 840 | C | ARG | A | 202 | −90.170 | 45.818 | 39.714 | 1.00 | 17.94 | C |
| ATOM | 841 | O | ARG | A | 202 | −90.962 | 45.558 | 38.806 | 1.00 | 17.91 | O |
| ATOM | 842 | N | VAL | A | 203 | −88.964 | 46.356 | 39.486 | 1.00 | 17.41 | N |
| ATOM | 843 | CA | VAL | A | 203 | −88.448 | 46.617 | 38.134 | 1.00 | 16.81 | C |
| ATOM | 844 | CB | VAL | A | 203 | −87.101 | 47.396 | 38.166 | 1.00 | 16.83 | C |
| ATOM | 845 | CG1 | VAL | A | 203 | −85.975 | 46.573 | 38.793 | 1.00 | 16.71 | C |
| ATOM | 846 | CG2 | VAL | A | 203 | −86.708 | 47.871 | 36.773 | 1.00 | 16.84 | C |
| ATOM | 847 | C | VAL | A | 203 | −88.316 | 45.328 | 37.317 | 1.00 | 16.28 | C |
| ATOM | 848 | O | VAL | A | 203 | −87.979 | 44.270 | 37.854 | 1.00 | 16.33 | O |
| ATOM | 849 | N | GLU | A | 204 | −88.609 | 45.448 | 36.022 | 1.00 | 15.49 | N |
| ATOM | 850 | CA | GLU | A | 204 | −88.630 | 44.330 | 35.081 | 1.00 | 14.75 | C |
| ATOM | 851 | CB | GLU | A | 204 | −90.082 | 43.993 | 34.751 | 1.00 | 14.56 | C |
| ATOM | 852 | CG | GLU | A | 204 | −90.296 | 42.896 | 33.719 | 1.00 | 14.36 | C |
| ATOM | 853 | CD | GLU | A | 204 | −91.761 | 42.748 | 33.358 | 1.00 | 14.30 | C |
| ATOM | 854 | OE1 | GLU | A | 204 | −92.603 | 42.695 | 34.275 | 1.00 | 14.22 | O |
| ATOM | 855 | OE2 | GLU | A | 204 | −92.076 | 42.695 | 32.157 | 1.00 | 14.37 | O |
| ATOM | 856 | C | GLU | A | 204 | −87.892 | 44.731 | 33.813 | 1.00 | 14.43 | C |
| ATOM | 857 | O | GLU | A | 204 | −88.101 | 45.836 | 33.304 | 1.00 | 14.07 | O |
| ATOM | 858 | N | TYR | A | 205 | −87.056 | 43.824 | 33.299 | 1.00 | 14.18 | N |
| ATOM | 859 | CA | TYR | A | 205 | −86.270 | 44.051 | 32.082 | 1.00 | 14.25 | C |
| ATOM | 860 | CB | TYR | A | 205 | −84.772 | 43.902 | 32.385 | 1.00 | 14.16 | C |
| ATOM | 861 | CG | TYR | A | 205 | −84.291 | 44.962 | 33.336 | 1.00 | 14.10 | C |
| ATOM | 862 | CD1 | TYR | A | 205 | −84.334 | 44.765 | 34.719 | 1.00 | 14.32 | C |
| ATOM | 863 | CE1 | TYR | A | 205 | −83.916 | 45.758 | 35.595 | 1.00 | 14.26 | C |
| ATOM | 864 | CZ | TYR | A | 205 | −83.461 | 46.970 | 35.083 | 1.00 | 14.33 | C |
| ATOM | 865 | OH | TYR | A | 205 | −83.040 | 47.981 | 35.915 | 1.00 | 14.51 | O |
| ATOM | 866 | CE2 | TYR | A | 205 | −83.419 | 47.178 | 33.717 | 1.00 | 14.24 | C |
| ATOM | 867 | CD2 | TYR | A | 205 | −83.840 | 46.187 | 32.858 | 1.00 | 14.10 | C |
| ATOM | 868 | C | TYR | A | 205 | −86.684 | 43.071 | 30.993 | 1.00 | 14.28 | C |
| ATOM | 869 | O | TYR | A | 205 | −86.848 | 41.883 | 31.263 | 1.00 | 14.03 | O |
| ATOM | 870 | N | LEU | A | 206 | −86.835 | 43.573 | 29.768 | 1.00 | 14.50 | N |
| ATOM | 871 | CA | LEU | A | 206 | −87.279 | 42.763 | 28.628 | 1.00 | 14.91 | C |
| ATOM | 872 | CB | LEU | A | 206 | −88.610 | 43.301 | 28.073 | 1.00 | 14.64 | C |
| ATOM | 873 | CG | LEU | A | 206 | −89.238 | 42.617 | 26.845 | 1.00 | 14.72 | C |
| ATOM | 874 | CD1 | LEU | A | 206 | −89.379 | 41.114 | 27.026 | 1.00 | 14.53 | C |
| ATOM | 875 | CD2 | LEU | A | 206 | −90.597 | 43.229 | 26.521 | 1.00 | 14.68 | C |
| ATOM | 876 | C | LEU | A | 206 | −86.233 | 42.727 | 27.522 | 1.00 | 15.20 | C |
| ATOM | 877 | O | LEU | A | 206 | −85.797 | 43.800 | 27.062 | 1.00 | 14.80 | O |
| ATOM | 878 | N | ASP | A | 207 | −85.823 | 41.539 | 27.136 | 1.00 | 15.94 | N |
| ATOM | 879 | CA | ASP | A | 207 | −85.168 | 41.278 | 25.858 | 1.00 | 16.57 | C |
| ATOM | 880 | CB | ASP | A | 207 | −84.117 | 40.153 | 25.986 | 1.00 | 16.69 | C |
| ATOM | 881 | CG | ASP | A | 207 | −82.837 | 40.591 | 26.685 | 1.00 | 16.69 | C |
| ATOM | 882 | OD1 | ASP | A | 207 | −82.629 | 41.801 | 26.918 | 1.00 | 16.46 | O |
| ATOM | 883 | OD2 | ASP | A | 207 | −82.018 | 39.697 | 26.988 | 1.00 | 16.54 | O |
| ATOM | 884 | C | ASP | A | 207 | −86.281 | 40.792 | 24.931 | 1.00 | 17.31 | C |
| ATOM | 885 | O | ASP | A | 207 | −86.667 | 39.626 | 24.996 | 1.00 | 16.71 | O |
| ATOM | 886 | N | ASP | A | 208 | −86.797 | 41.685 | 24.082 | 1.00 | 18.67 | N |
| ATOM | 887 | CA | ASP | A | 208 | −87.950 | 41.367 | 23.219 | 1.00 | 19.78 | C |
| ATOM | 888 | CB | ASP | A | 208 | −88.330 | 42.569 | 22.344 | 1.00 | 19.73 | C |
| ATOM | 889 | CG | ASP | A | 208 | −89.696 | 42.411 | 21.670 | 1.00 | 19.89 | C |
| ATOM | 890 | OD1 | ASP | A | 208 | −89.787 | 41.713 | 20.635 | 1.00 | 19.80 | O |
| ATOM | 891 | OD2 | ASP | A | 208 | −90.673 | 43.014 | 22.158 | 1.00 | 19.54 | O |
| ATOM | 892 | C | ASP | A | 208 | −87.639 | 40.144 | 22.352 | 1.00 | 21.14 | C |
| ATOM | 893 | O | ASP | A | 208 | −86.612 | 40.097 | 21.681 | 1.00 | 20.71 | O |
| ATOM | 894 | N | ARG | A | 209 | −88.521 | 39.148 | 22.394 | 1.00 | 23.02 | N |
| ATOM | 895 | CA | ARG | A | 209 | −88.288 | 37.880 | 21.685 | 1.00 | 24.37 | C |
| ATOM | 896 | CO | ARG | A | 209 | −89.266 | 36.790 | 22.165 | 1.00 | 25.73 | C |
| ATOM | 897 | CO | ARG | A | 209 | −90.739 | 37.014 | 21.841 | 1.00 | 26.88 | C |
| ATOM | 898 | Cl) | ARG | A | 209 | −91.588 | 35.860 | 22.359 | 1.00 | 28.16 | C |
| ATOM | 899 | NE | ARG | A | 209 | −91.319 | 34.611 | 21.637 | 1.00 | 29.00 | N |
| ATOM | 900 | CZ | ARG | A | 209 | −91.871 | 34.248 | 20.474 | 1.00 | 29.70 | C |
| ATOM | 901. | NH1 | ARG | A | 209 | −92.755 | 35.027 | 19.844 | 1.00 | 30.45 | N |
| ATOM | 902 | NH2 | ARG | A | 209 | −91.533 | 33.081 | 19.928 | 1.00 | 29.60 | N |
| ATOM | 903 | C | ARG | A | 209 | −88.312 | 38.006 | 20.153 | 1.00 | 23.98 | C |
| ATOM | 904 | O | ARG | A | 209 | −87.679 | 37.205 | 19.459 | 1.00 | 23.89 | O |
| ATOM | 905 | N | ASN | A | 210 | −89.018 | 39.018 | 19.644 | 1.00 | 24.03 | N |
| ATOM | 906 | CA | ASN | A | 210 | −89.208 | 39.222 | 18.204 | 1.00 | 23.80 | C |
| ATOM | 907 | CB | ASN | A | 210 | −90.671 | 39.585 | 17.925 | 1.00 | 24.46 | C |
| ATOM | 908 | CG | ASN | A | 210 | −91.647 | 38.554 | 18.472 | 1.00 | 24.80 | C |
| ATOM | 909 | OD1 | ASN | A | 210 | −92.622 | 38.904 | 19.131 | 1.00 | 25.49 | O |
| ATOM | 910 | ND2 | ASN | A | 210 | −91.384 | 37.277 | 18.206 | 1.00 | 24.91 | N |
| ATOM | 911 | C | ASN | A | 210 | −88.289 | 40.288 | 17.596 | 1.00 | 22.84 | C |
| ATOM | 912 | O | ASN | A | 210 | −87.735 | 40.071 | 16.517 | 1.00 | 23.28 | O |
| ATOM | 913 | N | THR | A | 211 | −88.145 | 41.432 | 18.273 | 1.00 | 21.54 | N |
| ATOM | 914 | CA | THR | A | 211 | −87.311 | 42.550 | 17.792 | 1.00 | 20.65 | C |
| ATOM | 915 | CB | THR | A | 211 | −88.010 | 43.909 | 18.024 | 1.00 | 20.60 | C |
| ATOM | 916 | OG1 | THR | A | 211 | −88.099 | 44.178 | 19.428 | 1.00 | 20.22 | O |
| ATOM | 917 | CG2 | THR | A | 211 | −89.417 | 43.911 | 17.414 | 1.00 | 20.52 | C |
| ATOM | 918 | C | THR | A | 211 | −85.914 | 42.624 | 18.431 | 1.00 | 19.91 | C |
| ATOM | 919 | O | THR | A | 211 | −85.072 | 43.408 | 17.979 | 1.00 | 19.37 | O |
| ATOM | 920 | N | THR | A | 212 | −85.688 | 41.854 | 19.499 | 1.00 | 18.91 | N |
| ATOM | 921 | CA | PHE | A | 212 | −84.396 | 41.810 | 20.212 | 1.00 | 18.65 | C |
| ATOM | 922 | CB | PHE | A | 212 | −83.247 | 41.372 | 19.270 | 1.00 | 18.94 | C |
| ATOM | 923 | CG | PHE | A | 212 | −83.308 | 39.916 | 18.852 | 1.00 | 19.45 | C |
| ATOM | 924 | CD1 | PHE | A | 212 | −84.463 | 39.364 | 18.290 | 1.00 | 19.49 | C |
| ATOM | 925 | OE1 | PHE | A | 212 | −84.504 | 38.03 | 17.909 | 1.00 | 19.59 | C |
| ATOM | 926 | CZ | PHE | A | 212 | −83.388 | 37.229 | 18.073 | 1.00 | 19.75 | C |
| ATOM | 927 | CE2 | PHE | A | 212 | −82.233 | 37.759 | 18.627 | 1.00 | 19.89 | C |
| ATOM | 928 | CD2 | PHE | A | 212 | −82.192 | 39.093 | 19.006 | 1.00 | 19.68 | C |
| ATOM | 929 | C | PHE | A | 212 | −84.054 | 43.121 | 20.953 | 1.00 | 18.05 | C |
| ATOM | 930 | O | PHE | A | 212 | −82.947 | 43.266 | 21.476 | 1.00 | 18.20 | O |
| ATOM | 931 | N | ARG | A | 213 | −85.010 | 44.051 | 21.033 | 1.00 | 17.21 | N |
| ATOM | 932 | CA | ARG | A | 213 | −84.774 | 45.373 | 21.627 | 1.00 | 16.60 | C |
| ATOM | 933 | CB | ARG | A | 213 | −85.738 | 46.423 | 21.061 | 1.00 | 16.72 | C |
| ATOM | 934 | CG | ARG | A | 213 | −85.376 | 46.931 | 19.677 | 1.00 | 16.83 | C |
| ATOM | 935 | CD | ARG | A | 213 | −86.426 | 47.917 | 19.198 | 1.00 | 16.91 | C |
| ATOM | 936 | NE | ARG | A | 213 | −86.188 | 48.395 | 17.837 | 1.00 | 16.98 | N |
| ATOM | 937 | CZ | ARG | A | 213 | −85.314 | 49.341 | 17.482 | 1.00 | 17.22 | C |
| ATOM | 938 | NH1 | ARG | A | 213 | −84.527 | 49.938 | 18.381 | 1.00 | 17.10 | N |
| ATOM | 939 | NH2 | ARG | A | 213 | −85.213 | 49.468 | 16.200 | 1.00 | 17.20 | N |
| ATOM | 940 | C | ARG | A | 213 | −84.937 | 45.309 | 23.135 | 1.00 | 15.67 | C |
| ATOM | 941 | O | ARG | A | 213 | −85.716 | 44.502 | 23.651 | 1.00 | 15.20 | O |
| ATOM | 942 | N | HIS | A | 214 | −84.195 | 46.174 | 23.825 | 1.00 | 14.67 | N |
| ATOM | 943 | CA | HIS | A | 214 | −84.193 | 46.224 | 25.278 | 1.00 | 14.13 | C |
| ATOM | 944 | CB | HIS | A | 214 | −82.812 | 46.595 | 25.816 | 1.00 | 14.01 | C |
| ATOM | 945 | CG | HIS | A | 214 | −81.714 | 45.715 | 25.325 | 1.00 | 14.08 | C |
| ATOM | 946 | ND1 | HIS | A | 214 | −80.387 | 46.077 | 25.401 | 1.00 | 14.21 | N |
| ATOM | 947 | CE1 | HIS | A | 214 | −79.644 | 45.115 | 24.885 | 1.00 | 14.12 | C |
| ATOM | 948 | NE2 | HIS | A | 214 | −80.442 | 44.149 | 24.467 | 1.00 | 13.99 | N |
| ATOM | 949 | CD2 | HIS | A | 214 | −81.742 | 44.499 | 24.732 | 1.00 | 14.14 | C |
| ATOM | 950 | C | HIS | A | 214 | −85.178 | 47.264 | 25.771 | 1.00 | 13.66 | C |
| ATOM | 951 | O | HIS | A | 214 | −85.304 | 48.326 | 25.172 | 1.00 | 13.22 | O |
| ATOM | 952 | N | SER | A | 215 | −85.863 | 46.950 | 26.869 | 1.00 | 13.39 | N |
| ATOM | 953 | CA | SER | A | 215 | −86.666 | 47.931 | 27.590 | 1.00 | 13.45 | C |
| ATOM | 954 | CB | SER | A | 215 | −86.078 | 48.034 | 27.001 | 1.00 | 13.37 | C |
| ATOM | 955 | OG | SER | A | 215 | −88.743 | 46.783 | 27.009 | 1.00 | 13.24 | O |
| ATOM | 956 | C | SER | A | 215 | −86.733 | 47.594 | 29.069 | 1.00 | 13.46 | C |
| ATOM | 957 | O | SER | A | 215 | −86.467 | 46.460 | 29.467 | 1.00 | 13.35 | O |
| ATOM | 958 | N | VAL | A | 216 | −87.089 | 48.599 | 29.867 | 1.00 | 13.52 | N |
| ATOM | 959 | CA | VAL | A | 216 | −87.199 | 48.473 | 31.325 | 1.00 | 13.76 | C |
| ATOM | 960 | CB | VAL | A | 216 | −86.061 | 49.226 | 32.068 | 1.00 | 13.79 | C |
| ATOM | 961 | CG1 | VAL | A | 216 | −85.989 | 50.709 | 31.691 | 1.00 | 13.60 | C |
| ATOM | 962 | CG2 | VAL | A | 216 | −86.193 | 49.055 | 33.575 | 1.00 | 13.64 | C |
| ATOM | 963 | C | VAL | A | 216 | −88.571 | 48.981 | 31.770 | 1.00 | 14.03 | C |
| ATOM | 964 | O | VAL | A | 216 | −88.998 | 50.063 | 31.343 | 1.00 | 13.76 | O |
| ATOM | 965 | N | VAL | A | 217 | −89.240 | 48.198 | 32.622 | 1.00 | 14.30 | N |
| ATOM | 966 | CA | VAL | A | 217 | −90.625 | 48.448 | 33.032 | 1.00 | 14.89 | C |
| ATOM | 967 | CB | VAL | A | 217 | −91.600 | 47.361 | 32.525 | 1.00 | 14.92 | C |
| ATOM | 968 | CG1 | VAL | A | 217 | −93.050 | 47.790 | 32.740 | 1.00 | 14.91 | C |
| ATOM | 969 | CG2 | VAL | A | 217 | −91.355 | 47.056 | 31.064 | 1.00 | 14.89 | C |
| ATOM | 970 | C | VAL | A | 217 | −90.770 | 48.452 | 34.543 | 1.00 | 15.48 | C |
| ATOM | 971 | O | VAL | A | 217 | −90.223 | 47.576 | 35.228 | 1.00 | 14.87 | O |
| ATOM | 972 | N | VAL | A | 218 | −91.532 | 49.428 | 35.045 | 1.00 | 15.85 | N |
| ATOM | 973 | CA | VAL | A | 218 | −91.923 | 49.475 | 36.450 | 1.00 | 16.56 | C |
| ATOM | 974 | CB | VAL | A | 218 | −92.2417 | 50.560 | 37.238 | 1.00 | 16.79 | C |
| ATOM | 975 | CG1 | VAL | A | 218 | −89.646 | 50.395 | 37.011 | 1.00 | 16.93 | C |
| ATOM | 976 | CG2 | VAL | A | 218 | −91.592 | 51.966 | 36.865 | 1.00 | 16.63 | C |
| ATOM | 977 | C | VAL | A | 218 | −93.433 | 49.700 | 36.562 | 1.00 | 16.80 | C |
| ATOM | 978 | O | VAL | A | 218 | −94.040 | 50.290 | 35.665 | 1.00 | 16.78 | O |
| ATOM | 979 | N | PRO | A | 219 | −94.047 | 49.201 | 37.650 | 1.00 | 17.50 | N |
| ATOM | 980 | CA | PRO | A | 219 | −95.444 | 49.507 | 37.968 | 1.00 | 17.52 | C |
| ATOM | 981 | CB | PRO | A | 219 | −95.636 | 48.866 | 39.341 | 1.00 | 17.56 | C |
| ATOM | 982 | CO | PRO | A | 219 | −94.662 | 47.745 | 39.375 | 1.00 | 17.48 | C |
| ATOM | 983 | CO | PRO | A | 219 | −93.511 | 48.123 | 38.502 | 1.00 | 17.31 | C |
| ATOM | 984 | C | PRO | A | 219 | −95.695 | 51.010 | 38.059 | 1.00 | 17.78 | C |
| ATOM | 985 | O | PRO | A | 219 | −94.878 | 51.731 | 38.638 | 1.00 | 17.11 | O |
| ATOM | 986 | N | TYR | A | 220 | −96.793 | 51.478 | 37.467 | 1.00 | 18.04 | N |
| ATOM | 987 | CA | TYR | A | 220 | −97.209 | 52.862 | 37.650 | 1.00 | 18.45 | C |
| ATOM | 988 | CB | TYR | A | 220 | −98.295 | 53.306 | 36.658 | 1.00 | 17.70 | C |
| ATOM | 989 | CG | TYR | A | 220 | −98.831 | 54.684 | 37.003 | 1.00 | 17.01 | C |
| ATOM | 990 | CD1 | TYR | A | 220 | −98.109 | 55.840 | 36.677 | 1.00 | 16.78 | C |
| ATOM | 991 | CE1 | TYR | A | 220 | −98.580 | 57.103 | 37.017 | 1.00 | 16.34 | C |
| ATOM | 992 | CZ | TYR | A | 220 | −99.785 | 57.224 | 37.701 | 1.00 | 16.30 | C |
| ATOM | 993 | OH | TYR | A | 220 | −100.262 | 58.470 | 38.044 | 1.00 | 15.77 | O |
| ATOM | 994 | CE2 | TYR | A | 220 | −100.502 | 56.092 | 38.056 | 1.00 | 16.40 | C |
| ATOM | 995 | CD2 | TYR | A | 220 | −100.028 | 54.835 | 37.707 | 1.00 | 16.59 | C |
| ATOM | 996 | C | TYR | A | 220 | −97.730 | 53.011 | 39.072 | 1.00 | 19.42 | C |
| ATOM | 997 | O | TYR | A | 220 | −98.553 | 52.219 | 39.528 | 1.00 | 19.35 | O |
| ATOM | 998 | N | PRO | A | 221 | −97.235 | 54.034 | 39.757 | 1.00 | 21.00 | N |
| ATOM | 999 | CA | PRO | A | 221 | −97.655 | 54.358 | 41.107 | 1.00 | 22.02 | C |
| ATOM | 1000 | CB | PRO | A | 221 | −96.444 | 54.406 | 42.047 | 1.00 | 23.11 | C |
| ATOM | 1001 | CG | PRO | A | 221 | −95.540 | 53.171 | 42.019 | 1.00 | 23.90 | C |
| ATOM | 1002 | CD | PRO | A | 221 | −96.026 | 52.030 | 42.900 | 1.00 | 24.54 | C |
| ATOM | 1003 | OE1 | PRO | A | 221 | −96.323 | 52.279 | 44.087 | 1.00 | 24.83 | O |
| ATOM | 1004 | OE2 | PRO | A | 221 | −96.067 | 50.874 | 42.412 | 1.00 | 25.37 | O |
| ATOM | 1005 | C | PRO | A | 221 | −98.285 | 55.744 | 41.010 | 1.00 | 22.22 | C |
| ATOM | 1006 | O | PRO | A | 221 | −97.704 | 56.624 | 40.369 | 1.00 | 21.41 | O |
| ATOM | 1007 | N | PRO | A | 222 | −99.470 | 55.950 | 41.626 | 1.00 | 22.39 | N |
| ATOM | 1008 | CA | PRO | A | 222 | −100.015 | 57.311 | 41.635 | 1.00 | 22.63 | C |
| ATOM | 1009 | CB | PRO | A | 222 | −101.394 | 57.141 | 42.282 | 1.00 | 22.66 | C |
| ATOM | 1010 | CG | PRO | A | 222 | −101.288 | 55.902 | 43.100 | 1.00 | 22.54 | C |
| ATOM | 1011 | CD | PRO | A | 222 | −100.323 | 55.008 | 42.378 | 1.00 | 22.55 | C |
| ATOM | 1012 | C | PRO | A | 222 | −99.140 | 58.249 | 42.468 | 1.00 | 22.90 | C |
| ATOM | 1013 | O | PRO | A | 222 | −98.354 | 57.771 | 43.293 | 1.00 | 22.55 | O |
| ATOM | 1014 | N | PRO | A | 223 | −99.256 | 59.573 | 42.253 | 1.00 | 23.76 | N |
| ATOM | 1015 | CA | PRO | A | 223 | −98.493 | 60.469 | 43.117 | 1.00 | 24.38 | C |
| ATOM | 1016 | CB | PRO | A | 223 | −98.849 | 61.870 | 42.591 | 1.00 | 24.23 | C |
| ATOM | 1017 | CG | PRO | A | 223 | −99.406 | 61.651 | 41.225 | 1.00 | 24.10 | C |
| ATOM | 1018 | CD | PRO | A | 223 | −100.085 | 60.318 | 41.290 | 1.00 | 23.95 | C |
| ATOM | 1019 | C | PRO | A | 223 | −98.929 | 60.303 | 44.569 | 1.00 | 25.11 | C |
| ATOM | 1020 | O | PRO | A | 223 | −100.103 | 60.026 | 44.834 | 1.00 | 25.10 | O |
| ATOM | 1021 | N | GLU | A | 224 | −97.986 | 60.440 | 45.494 | 1.00 | 26.00 | N |
| ATOM | 1022 | CA | GLU | A | 224 | −98.298 | 60.358 | 46.918 | 1.00 | 26.69 | C |
| ATOM | 1023 | CB | GLU | A | 224 | −97.009 | 60.323 | 47.744 | 1.00 | 27.64 | C |
| ATOM | 1024 | CG | GLU | A | 224 | −96.192 | 59.049 | 47.525 | 1.00 | 28.15 | C |
| ATOM | 1025 | CD | GLU | A | 224 | −94.806 | 59.094 | 48.149 | 1.00 | 28.57 | C |
| ATOM | 1026 | OE1 | GLU | A | 224 | −94.341 | 58.038 | 48.632 | 1.00 | 29.30 | O |
| ATOM | 1027 | OE2 | GLU | A | 224 | −94.172 | 60.171 | 48.151 | 1.00 | 28.47 | O |
| ATOM | 1028 | C | GLU | A | 224 | −99.192 | 61.543 | 47.301 | 1.00 | 26.87 | C |
| ATOM | 1029 | O | GLU | A | 224 | −99.254 | 62.537 | 46.570 | 1.00 | 26.18 | O |
| ATOM | 1030 | N | VAL | A | 225 | −99.899 | 61.421 | 48.424 | 1.00 | 27.06 | N |
| ATOM | 1031 | CA | VAL | A | 225 | −100.908 | 62.415 | 48.830 | 1.00 | 27.24 | C |
| ATOM | 1032 | CB | VAL | A | 225 | −101.585 | 62.037 | 50.175 | 1.00 | 27.49 | C |
| ATOM | 1033 | CG1 | VAL | A | 225 | −102.406 | 63.197 | 50.742 | 1.00 | 27.59 | C |
| ATOM | 1034 | CG2 | VAL | A | 225 | −102.464 | 60.803 | 49.996 | 1.00 | 27.59 | C |
| ATOM | 1035 | C | VAL | A | 225 | −100.292 | 63.817 | 48.901 | 1.00 | 27.04 | C |
| ATOM | 1036 | O | VAL | A | 225 | −99.211 | 64.002 | 49.461 | 1.00 | 26.27 | O |
| ATOM | 1037 | N | GLY | A | 226 | −100.981 | 64.784 | 48.300 | 1.00 | 27.12 | N |
| ATOM | 1038 | CA | GLY | A | 226 | −100.489 | 66.156 | 48.213 | 1.00 | 27.30 | C |
| ATOM | 1039 | C | GLY | A | 226 | −99.348 | 66.384 | 47.229 | 1.00 | 27.14 | C |
| ATOM | 1040 | O | GLY | A | 226 | −98.695 | 67.429 | 47.294 | 1.00 | 27.24 | O |
| ATOM | 1041 | N | SER | A | 227 | −99.098 | 65.421 | 46.333 | 1.00 | 26.64 | N |
| ATOM | 1042 | CA | SER | A | 227 | −98.124 | 65.572 | 45.247 | 1.00 | 26.08 | C |
| ATOM | 1043 | CB | SER | A | 227 | −97.128 | 64.414 | 45.224 | 1.00 | 26.21 | C |
| ATOM | 1044 | OG | SER | A | 227 | −96.099 | 64.652 | 44.274 | 1.00 | 26.37 | O |
| ATOM | 1045 | C | SFR | A | 227 | −98.847 | 65.632 | 43.911 | 1.00 | 25.99 | C |
| ATOM | 1046 | O | SER | A | 227 | −99.955 | 65.103 | 43.760 | 1.00 | 25.73 | O |
| ATOM | 1047 | N | ASP | A | 228 | −98.192 | 66.265 | 42.941 | 1.00 | 25.50 | N |
| ATOM | 1048 | CA | ASP | A | 228 | −98.771 | 66.506 | 41.622 | 1.00 | 25.17 | C |
| ATOM | 1049 | CB | ASP | A | 228 | −98.366 | 67.895 | 41.118 | 1.00 | 25.94 | C |
| ATOM | 1050 | CG | ASP | A | 228 | −98.680 | 69.005 | 42.112 | 1.00 | 26.96 | C |
| ATOM | 1051 | OD1 | ASP | A | 228 | −99.509 | 68.792 | 43.021 | 1.00 | 28.10 | O |
| ATOM | 1052 | OD2 | ASP | A | 228 | −98.095 | 70.100 | 41.975 | 1.00 | 27.93 | O |
| ATOM | 1053 | C | ASP | A | 228 | −98.361 | 65.461 | 40.582 | 1.00 | 23.76 | C |
| ATOM | 1054 | O | ASP | A | 228 | −99.048 | 65.9308 | 39.574 | 1.00 | 22.77 | O |
| ATOM | 1055 | N | CYS | A | 228 | −97.250 | 64.758 | 40.816 | 1.00 | 22.53 | N |
| ATOM | 1056 | CA | CYS | A | 229 | −96.700 | 63.834 | 39.817 | 1.00 | 22.13 | C |
| ATOM | 1057 | CB | CYS | A | 229 | −95.749 | 64.597 | 38.895 | 1.00 | 21.92 | C |
| ATOM | 1058 | SG | CYS | A | 229 | −94.169 | 65.023 | 39.661 | 1.00 | 21.35 | S |
| ATOM | 1059 | C | CYS | A | 229 | −95.961 | 62.657 | 40.436 | 1.00 | 21.39 | C |
| ATOM | 1060 | O | CYS | A | 229 | −95.586 | 62.697 | 41.606 | 1.00 | 21.70 | O |
| ATOM | 1061 | N | THR | A | 230 | −95.764 | 61.617 | 39.625 | 1.00 | 20.92 | N |
| ATOM | 1062 | CA | THR | A | 230 | −94.986 | 60.440 | 39.996 | 1.00 | 20.48 | C |
| ATOM | 1063 | CB | THR | A | 230 | −95.617 | 59.144 | 39.449 | 1.00 | 20.78 | C |
| ATOM | 1064 | OG1 | THR | A | 230 | −97.029 | 59.144 | 39.669 | 1.00 | 21.69 | O |
| ATOM | 1065 | CG2 | THR | A | 230 | −94.976 | 57.924 | 40.099 | 1.00 | 20.96 | C |
| ATOM | 1066 | C | THR | A | 230 | −93.603 | 60.562 | 39.379 | 1.00 | 19.47 | C |
| ATOM | 1067 | O | THR | A | 230 | −93.489 | 60.848 | 38.191 | 1.00 | 19.65 | O |
| ATOM | 1068 | N | THR | A | 231 | −92.567 | 60.299 | 40.173 | 1.00 | 18.31 | N |
| ATOM | 1069 | CA | THR | A | 231 | −91.177 | 60.370 | 39.715 | 1.00 | 17.57 | C |
| ATOM | 1070 | CB | THR | A | 231 | −90.337 | 61.219 | 40.694 | 1.00 | 17.52 | C |
| ATOM | 1071 | OG1 | THR | A | 231 | −90.696 | 62.597 | 40.538 | 1.00 | 17.51 | C |
| ATOM | 1072 | CG2 | THR | A | 231 | −88.831 | 61.070 | 40.443 | 1.00 | 17.60 | C |
| ATOM | 1073 | C | THR | A | 231 | −90.583 | 58.965 | 39.582 | 1.00 | 16.91 | C |
| ATOM | 1074 | O | THR | A | 231 | −90.729 | 58.148 | 40.491 | 1.00 | 16.59 | O |
| ATOM | 1075 | N | ILE | A | 232 | −89.934 | 58.700 | 38.440 | 1.00 | 16.32 | N |
| ATOM | 1076 | CA | ILE | A | 232 | −89.100 | 57.506 | 38.241 | 1.00 | 15.84 | C |
| ATOM | 1077 | CB | ILE | A | 232 | −89.416 | 56.770 | 36.921 | 1.00 | 15.69 | C |
| ATOM | 1078 | CG1 | ILE | A | 232 | −90.921 | 56.497 | 36.803 | 1.00 | 15.63 | C |
| ATOM | 1079 | CD1 | ILE | A | 232 | −91.304 | 55.579 | 35.661 | 1.00 | 15.56 | C |
| ATOM | 1080 | CG2 | ILE | A | 232 | −88.608 | 55.471 | 36.825 | 1.00 | 15.66 | C |
| ATOM | 1081 | C | ILE | A | 232 | −87.636 | 57.927 | 38.193 | 1.00 | 15.55 | C |
| ATOM | 1082 | O | ILE | A | 232 | −87.280 | 58.853 | 37.456 | 1.00 | 15.12 | O |
| ATOM | 1083 | N | HIS | A | 233 | −86.793 | 57.229 | 38.952 | 1.00 | 15.19 | N |
| ATOM | 1084 | CA | HIS | A | 233 | −85.346 | 57.442 | 38.897 | 1.00 | 15.18 | C |
| ATOM | 1085 | CB | HIS | A | 233 | −84.710 | 57.267 | 40.279 | 1.00 | 15.46 | C |
| ATOM | 1086 | CG | HIS | A | 233 | −85.099 | 58.329 | 41.261 | 1.00 | 15.92 | C |
| ATOM | 1087 | ND1 | HIS | A | 233 | −85.146 | 58.108 | 42.621 | 1.00 | 16.51 | N |
| ATOM | 1088 | CE1 | HIS | A | 233 | −85.522 | 59.219 | 43.232 | 1.00 | 16.31 | C |
| ATOM | 1089 | NE2 | HIS | A | 233 | −85.722 | 60.150 | 42.318 | 1.00 | 16.15 | N |
| ATOM | 1090 | CD2 | HIS | A | 233 | −85.467 | 59.619 | 41.077 | 1.00 | 16.05 | C |
| ATOM | 1091 | C | HIS | A | 233 | −84.717 | 55.483 | 37.889 | 1.00 | 14.80 | C |
| ATOM | 1092 | O | HIS | A | 233 | −84.557 | 55.294 | 38.172 | 1.00 | 14.59 | O |
| ATOM | 1093 | N | TYR | A | 234 | −84.383 | 56.997 | 36.706 | 1.00 | 14.39 | N |
| ATOM | 1094 | CA | TYR | A | 234 | −83.645 | 56.214 | 35.715 | 1.00 | 14.30 | C |
| ATOM | 1095 | CB | TYR | A | 234 | −84.012 | 56.635 | 34.287 | 1.00 | 14.02 | C |
| ATOM | 1096 | CG | TYR | A | 234 | −85.408 | 56.200 | 33.886 | 1.00 | 13.67 | C |
| ATOM | 1097 | CD1 | TYR | A | 234 | −85.684 | 54.860 | 33.605 | 1.00 | 13.50 | C |
| ATOM | 1098 | CE1 | TYR | A | 234 | −86.955 | 54.448 | 33.241 | 1.00 | 13.24 | C |
| ATOM | 1099 | CZ | TYR | A | 234 | −87.982 | 55.370 | 33.164 | 1.00 | 13.27 | C |
| ATOM | 1100 | OH | TYR | A | 234 | −89.243 | 54.936 | 32.803 | 1.00 | 12.93 | O |
| ATOM | 1101 | CE2 | TYR | A | 234 | −87.740 | 56.708 | 33.438 | 1.00 | 13.18 | C |
| ATOM | 1102 | CD2 | TYR | A | 234 | −86.456 | 57.115 | 33.799 | 1.00 | 13.49 | C |
| ATOM | 1103 | C | TYR | A | 234 | −82.137 | 56.323 | 35.957 | 1.00 | 14.63 | C |
| ATOM | 1104 | O | TYR | A | 234 | −81.672 | 57.205 | 36.678 | 1.00 | 14.71 | O |
| ATOM | 1105 | N | ASN | A | 235 | −81.399 | 55.375 | 35.393 | 1.00 | 15.16 | N |
| ATOM | 1106 | CA | ASN | A | 235 | −79.940 | 55.410 | 35.345 | 1.00 | 15.53 | C |
| ATOM | 1107 | CB | ASN | A | 235 | −79.304 | 54.380 | 36.292 | 1.00 | 15.77 | C |
| ATOM | 1108 | CG | ASN | A | 235 | −79.441 | 54.730 | 37.763 | 1.00 | 16.18 | C |
| ATOM | 1109 | CD1 | ASN | A | 235 | −79.231 | 53.867 | 38.619 | 1.00 | 17.09 | O |
| ATOM | 1110 | ND2 | ASN | A | 235 | −79.768 | 55.980 | 38.074 | 1.00 | 16.29 | N |
| ATOM | 1111 | C | ASN | A | 235 | −79.575 | 55.029 | 33.933 | 1.00 | 15.23 | C |
| ATOM | 1112 | O | ASN | A | 235 | −79.979 | 53.950 | 33.466 | 1.00 | 15.25 | O |
| ATOM | 1113 | N | TYR | A | 236 | −78.838 | 55.900 | 33.248 | 1.00 | 15.74 | N |
| ATOM | 1114 | CA | TYR | A | 236 | −78.305 | 55.587 | 31.927 | 1.00 | 15.88 | C |
| ATOM | 1115 | CB | TYR | A | 236 | −78.383 | 56.801 | 31.011 | 1.00 | 15.87 | C |
| ATOM | 1116 | CG | TYR | A | 236 | −79.803 | 57.183 | 30.675 | 1.00 | 15.73 | C |
| ATOM | 1117 | CD1 | TYR | A | 236 | −80.851 | 57.904 | 31.578 | 1.00 | 15.57 | C |
| ATOM | 1118 | CE1 | TYR | A | 236 | −81.890 | 58.250 | 31.279 | 1.00 | 15.76 | C |
| ATOM | 1119 | CZ | TYR | A | 236 | −82.441 | 57.859 | 30.069 | 1.00 | 15.50 | C |
| ATOM | 1120 | OH | TYR | A | 236 | −83.735 | 58.207 | 29.785 | 1.00 | 15.48 | O |
| ATOM | 1121 | CE2 | TYR | A | 236 | −81.689 | 57.135 | 29.155 | 1.00 | 15.49 | C |
| ATOM | 1122 | CD2 | TYR | A | 236 | −80.383 | 56.795 | 29.463 | 1.00 | 15.50 | C |
| ATOM | 1123 | C | TYR | A | 236 | −76.869 | 55.112 | 32.112 | 1.00 | 16.41 | C |
| ATOM | 1124 | O | TYR | A | 236 | −76.057 | 55.794 | 32.747 | 1.00 | 16.23 | O |
| ATOM | 1125 | N | MET | A | 237 | −76.574 | 53.941 | 31.554 | 1.00 | 17.17 | N |
| ATOM | 1126 | CA | MET | A | 237 | −75.373 | 53.180 | 31.897 | 1.00 | 17.82 | C |
| ATOM | 1127 | CB | MET | A | 237 | −75.803 | 51.753 | 32.348 | 1.00 | 17.80 | C |
| ATOM | 1128 | CG | MET | A | 237 | −76.826 | 51.640 | 33.376 | 1.00 | 17.64 | C |
| ATOM | 1129 | SD | MET | A | 237 | −76.336 | 52.407 | 34.931 | 1.00 | 17.32 | S |
| ATOM | 1130 | CE | MET | A | 237 | −74.765 | 51.608 | 35.270 | 1.00 | 17.22 | C |
| ATOM | 1131 | C | MET | A | 237 | −74.301 | 53.158 | 30.797 | 1.00 | 18.56 | C |
| ATOM | 1132 | O | MET | A | 237 | −73.337 | 52.388 | 30.886 | 1.00 | 18.83 | O |
| ATOM | 1133 | N | CYS | A | 238 | −74.459 | 54.021 | 29.790 | 1.00 | 19.35 | N |
| ATOM | 1134 | CA | CYS | A | 238 | −73.581 | 54.091 | 28.614 | 1.00 | 20.14 | C |
| ATOM | 1135 | CB | CYS | A | 238 | −73.642 | 52.769 | 27.827 | 1.00 | 20.00 | C |
| ATOM | 1136 | SG | CYS | A | 238 | −72.643 | 52.668 | 26.327 | 1.00 | 20.15 | C |
| ATOM | 1137 | C | CYS | A | 238 | −74.044 | 55.265 | 27.732 | 1.00 | 21.02 | C |
| ATOM | 1138 | O | CYS | A | 238 | −75.164 | 55.741 | 27.886 | 1.00 | 21.10 | O |
| ATOM | 1139 | N | ASN | A | 239 | −73.192 | 55.743 | 26.826 | 1.00 | 22.21 | N |
| ATOM | 1140 | CA | ASN | A | 239 | −73.605 | 56.733 | 25.800 | 1.00 | 22.95 | O |
| ATOM | 1141 | CB | ASN | A | 239 | −72.690 | 57.975 | 25.820 | 1.00 | 22.96 | C |
| ATOM | 1142 | CG | ASN | A | 239 | −73.231 | 59.145 | 24.991 | 1.00 | 23.52 | C |
| ATOM | 1143 | OD1 | ASN | A | 239 | −72.473 | 59.826 | 24.289 | 1.00 | 23.67 | O |
| ATOM | 1144 | ND2 | ASN | A | 239 | −74.537 | 59.378 | 25.058 | 1.00 | 23.11 | N |
| ATOM | 1145 | C | ASN | A | 239 | −73.604 | 56.092 | 24.407 | 1.00 | 23.38 | C |
| ATOM | 1146 | O | ASN | A | 239 | −73.544 | 56.798 | 23.400 | 1.00 | 24.01 | O |
| ATOM | 1147 | N | SER | A | 240 | −73.674 | 54.759 | 24.359 | 1.00 | 23.96 | N |
| ATOM | 1148 | CA | SER | A | 240 | −73.546 | 53.954 | 23.129 | 1.00 | 23.95 | C |
| ATOM | 1149 | CB | SER | A | 240 | −74.674 | 54.254 | 22.130 | 1.00 | 24.04 | C |
| ATOM | 1150 | OG | SER | A | 240 | −74.495 | 53.497 | 20.946 | 1.00 | 24.02 | O |
| ATOM | 1151 | C | SER | A | 240 | −72.183 | 54.057 | 22.434 | 1.00 | 24.01 | C |
| ATOM | 1152 | O | SER | A | 240 | −71.467 | 53.061 | 22.312 | 1.00 | 24.06 | O |
| ATOM | 1153 | N | SER | A | 241 | −71.846 | 55.259 | 21.972 | 1.00 | 24.20 | N |
| ATOM | 1154 | CA | SER | A | 241 | −70.624 | 55.518 | 21.218 | 1.00 | 24.67 | C |
| ATOM | 1155 | CB | SER | A | 241 | −70.943 | 56.565 | 20.143 | 1.00 | 25.19 | C |
| ATOM | 1156 | OG | SER | A | 241 | −71.392 | 57.777 | 20.731 | 1.00 | 25.20 | O |
| ATOM | 1157 | C | SER | A | 241 | −69.440 | 55.996 | 22.090 | 1.00 | 24.56 | C |
| ATOM | 1158 | O | SER | A | 241 | −68.633 | 56.807 | 21.634 | 1.00 | 24.54 | O |
| ATOM | 1159 | N | CYS | A | 242 | −69.316 | 55.477 | 23.316 | 1.00 | 24.93 | N |
| ATOM | 1160 | CA | CYS | A | 242 | −68.317 | 55.982 | 24.286 | 1.00 | 25.36 | C |
| ATOM | 1161 | CB | CYS | A | 242 | −68.484 | 55.323 | 25.653 | 1.00 | 24.29 | C |
| ATOM | 1162 | SG | CYS | A | 242 | −70.178 | 55.106 | 26.204 | 1.00 | 23.61 | S |
| ATOM | 1163 | C | CYS | A | 242 | −66.859 | 55.796 | 23.875 | 1.00 | 26.80 | C |
| ATOM | 1164 | O | CYS | A | 242 | −66.005 | 56.591 | 24.276 | 1.00 | 27.79 | O |
| ATOM | 1165 | N | MET | A | 243 | −66.578 | 54.739 | 23.113 | 1.00 | 28.40 | N |
| ATOM | 1166 | CA | MET | A | 243 | −65.217 | 54.428 | 22.658 | 1.00 | 29.70 | C |
| ATOM | 1167 | CB | MET | A | 243 | −64.753 | 53.095 | 23.261 | 1.00 | 30.66 | C |
| ATOM | 1168 | CG | MET | A | 243 | −65.447 | 51.861 | 22.711 | 1.00 | 31.33 | C |
| ATOM | 1169 | SD | MET | A | 243 | −65.640 | 50.560 | 23.939 | 1.00 | 32.58 | C |
| ATOM | 1170 | CE | MET | A | 243 | −64.428 | 49.378 | 23.367 | 1.00 | 31.90 | C |
| ATOM | 1171 | C | MET | A | 243 | −65.090 | 54.428 | 21.125 | 1.00 | 30.09 | C |
| ATOM | 1172 | O | MET | A | 243 | −64.216 | 53.759 | 20.569 | 1.00 | 30.33 | O |
| ATOM | 1173 | N | GLY | A | 244 | −65.948 | 55.197 | 20.454 | 1.00 | 30.35 | N |
| ATOM | 1174 | CA | GLY | A | 244 | −65.920 | 55.329 | 18.998 | 1.00 | 30.83 | C |
| ATOM | 1175 | C | GLY | A | 244 | −66.941 | 54.447 | 18.303 | 1.00 | 30.70 | C |
| ATOM | 1176 | O | GLY | A | 244 | −67.905 | 53.993 | 18.926 | 1.00 | 30.61 | O |
| ATOM | 1177 | N | GLY | A | 245 | −66.733 | 54.227 | 17.003 | 1.00 | 30.66 | N |
| ATOM | 1178 | CA | GLY | A | 245 | −67.671 | 53.477 | 16.154 | 1.00 | 30.63 | C |
| ATOM | 1179 | C | GLY | A | 245 | −68.290 | 54.319 | 15.049 | 1.00 | 30.69 | C |
| ATOM | 1180 | O | GLY | A | 245 | −68.155 | 55.544 | 15.037 | 1.00 | 30.33 | O |
| ATOM | 1181 | N | MET | A | 246 | −68.970 | 53.651 | 14.119 | 1.00 | 30.06 | N |
| ATOM | 1182 | CA | MET | A | 246 | −69.726 | 54.322 | 13.057 | 1.00 | 29.85 | C |
| ATOM | 1183 | CB | MET | A | 246 | −70.182 | 53.301 | 12.010 | 1.00 | 30.07 | C |
| ATOM | 1184 | CG | MET | A | 246 | −70.882 | 53.891 | 10.790 | 1.00 | 30.06 | C |
| ATOM | 1185 | SD | MET | A | 246 | −71.322 | 52.631 | 9.578 | 1.00 | 29.83 | S |
| ATOM | 1186 | CE | MET | A | 246 | −72.703 | 51.807 | 10.355 | 1.00 | 30.08 | C |
| ATOM | 1187 | C | MET | A | 246 | −70.938 | 55.067 | 13.613 | 1.00 | 29.87 | C |
| ATOM | 1188 | O | MET | A | 246 | −71.204 | 56.204 | 13.215 | 1.00 | 29.98 | O |
| ATOM | 1189 | N | ASN | A | 247 | −71−664 | 54.422 | 14.527 | 1.00 | 30.03 | N |
| ATOM | 1190 | CA | ASN | A | 247 | −72.911 | 54.965 | 15.063 | 1.00 | 30.10 | C |
| ATOM | 1192 | CB | ASN | A | 247 | −73.742 | 53.846 | 15.716 | 1.00 | 29.96 | C |
| ATOM | 1192 | CG | ASN | A | 247 | −75.149 | 54.288 | 16.096 | 1.00 | 30.08 | C |
| ATOM | 1193 | OD1 | ASN | A | 247 | −75.491 | 55.470 | 16.042 | 1.00 | 30.46 | O |
| ATOM | 1194 | ND1 | ASN | A | 247 | −75.977 | 53.325 | 16.484 | 1.00 | 30.07 | N |
| ATOM | 1195 | C | ASN | A | 247 | −72.654 | 56.1.00 | 16.058 | 1.00 | 30.18 | C |
| ATOM | 1196 | O | ASN | A | 247 | −72.304 | 55.853 | 17.214 | 1.00 | 30.09 | O |
| ATOM | 1197 | N | ARG | A | 248 | −72.833 | 57.336 | 15.588 | 1.00 | 30.06 | N |
| ATOM | 1198 | CA | ARG | A | 248 | −72.839 | 56.532 | 16.442 | 1.00 | 30.04 | C |
| ATOM | 1199 | CB | ARG | A | 248 | −71.961 | 59.620 | 15.837 | 1.00 | 30.57 | C |
| ATOM | 1200 | CG | ARG | A | 248 | −70.498 | 59.281 | 15.729 | 1.00 | 31.16 | C |
| ATOM | 1201 | CD | ARG | A | 248 | −69.750 | 59.562 | 17.020 | 1.00 | 31.59 | C |
| ATOM | 1202 | NE | ARG | A | 248 | −66.370 | 59.165 | 16.811 | 1.00 | 32.18 | N |
| ATOM | 1203 | CZ | ARG | A | 248 | −67.935 | 57.912 | 16.866 | 1.00 | 32.37 | C |
| ATOM | 1204 | NH1 | ARG | A | 248 | −66.652 | 57.662 | 16.630 | 1.00 | 32.59 | N |
| ATOM | 1205 | NO2 | ARG | A | 248 | −68.771 | 56.908 | 17.141 | 1.00 | 32.92 | N |
| ATOM | 1206 | C | ARG | A | 248 | −74.241 | 59.115 | 16.596 | 1.00 | 29.32 | C |
| ATOM | 1207 | O | ARG | A | 248 | −74.389 | 60.312 | 16.866 | 1.00 | 30.21 | O |
| ATOM | 1208 | N | SER | A | 249 | −75.273 | 58.293 | 16.426 | 1.00 | 28.32 | N |
| ATOM | 1209 | CA | SER | A | 249 | −76.637 | 58.779 | 16.560 | 1.00 | 27.25 | C |
| ATOM | 1210 | CB | SER | A | 249 | −77.639 | 57.697 | 16.185 | 1.00 | 27.38 | C |
| ATOM | 1211 | OG | SER | A | 249 | −78.952 | 58.216 | 16.170 | 1.00 | 27.71 | O |
| ATOM | 1212 | C | SER | A | 249 | −76.882 | 59.225 | 17.999 | 1.00 | 26.02 | C |
| ATOM | 1213 | O | SER | A | 249 | −76.434 | 58.552 | 18.932 | 1.00 | 25.86 | O |
| ATOM | 1214 | N | PRO | A | 250 | −77.559 | 60.376 | 18.184 | 1.00 | 24.76 | N |
| ATOM | 1215 | CA | PRO | A | 250 | −78.095 | 60.671 | 19.511 | 1.00 | 23.91 | C |
| ATOM | 1216 | CB | PRO | A | 250 | −78.716 | 62.066 | 19.347 | 1.00 | 24.15 | C |
| ATOM | 1217 | CG | PRO | A | 250 | −76.049 | 62.655 | 18.154 | 1.00 | 24.46 | C |
| ATOM | 1218 | CD | PRO | A | 250 | −77.755 | 61.500 | 17.249 | 1.00 | 24.67 | C |
| ATOM | 1219 | C | PRO | A | 250 | −79.158 | 59.641 | 19.891 | 1.00 | 22.84 | C |
| ATOM | 1220 | O | PRO | A | 250 | −79.842 | 59.111 | 19.013 | 1.00 | 22.78 | O |
| ATOM | 1221 | N | ILE | A | 251 | −79.279 | 59.356 | 21.184 | 1.00 | 21.50 | N |
| ATOM | 1222 | CA | ILE | A | 251 | −80.233 | 56.362 | 21.672 | 1.00 | 20.17 | C |
| ATOM | 1223 | CB | ILE | A | 251 | −79.644 | 57.530 | 22.831 | 1.00 | 20.23 | C |
| ATOM | 1224 | CG1 | ILE | A | 251 | −78.332 | 56.856 | 22.398 | 1.00 | 20.27 | C |
| ATOM | 1225 | CD1 | ILE | A | 251 | −77.377 | 56.580 | 23.539 | 1.00 | 20.20 | C |
| ATOM | 1226 | CG2 | ILE | A | 251 | −80.643 | 56.475 | 23.303 | 1.00 | 20.29 | C |
| ATOM | 1227 | C | ILE | A | 251 | −81.505 | 59.071 | 22.133 | 1.00 | 19.06 | C |
| ATOM | 1228 | O | ILE | A | 251 | −81.436 | 60.092 | 22.823 | 1.00 | 18.92 | O |
| ATOM | 1229 | N | LEU | A | 252 | −82.652 | 58.523 | 21.727 | 1.00 | 17.53 | N |
| ATOM | 1230 | CA | LEU | A | 252 | −83.971 | 56.961 | 22.187 | 1.00 | 16.31 | C |
| ATOM | 1231 | CB | LEU | A | 252 | −84.939 | 59.027 | 21.005 | 1.00 | 16.19 | C |
| ATOM | 1232 | CG | LEU | A | 252 | −86.314 | 59.650 | 21.279 | 1.00 | 16.10 | C |
| ATOM | 1233 | CD1 | LEU | A | 252 | −86.222 | 61.170 | 21.257 | 1.00 | 16.07 | C |
| ATOM | 1234 | CD2 | LEU | A | 252 | −87.361 | 59.176 | 20.285 | 1.00 | 16.15 | C |
| ATOM | 1235 | C | LEU | A | 252 | −84.510 | 57.961 | 23.214 | 1.00 | 15.59 | C |
| ATOM | 1236 | O | LEU | A | 252 | −84.508 | 56.759 | 22.952 | 1.00 | 14.86 | O |
| ATOM | 1237 | N | THR | A | 253 | −84.970 | 58.480 | 24.365 | 1.00 | 14.75 | N |
| ATOM | 1238 | CA | THR | A | 253 | −85.694 | 57.644 | 25.346 | 1.00 | 14.43 | C |
| ATOM | 1239 | CB | THR | A | 253 | −85.378 | 58.063 | 26.793 | 1.00 | 14.37 | C |
| ATOM | 1240 | OG1 | THR | A | 253 | −84.010 | 57.757 | 27.092 | 1.00 | 14.22 | O |
| ATOM | 1241 | CG2 | THR | A | 253 | −86.279 | 57.330 | 27.798 | 1.00 | 14.28 | C |
| ATOM | 1242 | C | THR | A | 253 | −87.193 | 57.798 | 25.089 | 1.00 | 14.32 | C |
| ATOM | 1243 | O | THR | A | 253 | −87.723 | 58.896 | 25.208 | 1.00 | 14.34 | O |
| ATOM | 1244 | N | ILE | A | 254 | −87.852 | 56.698 | 24.735 | 1.00 | 14.11 | N |
| ATOM | 1245 | CA | ILE | A | 254 | −89.303 | 56.666 | 24.527 | 1.00 | 14.04 | C |
| ATOM | 1246 | CB | ILE | A | 254 | −89.684 | 55.825 | 23.286 | 1.00 | 14.10 | C |
| ATOM | 1247 | CG1 | ILE | A | 254 | −88.869 | 56.267 | 22.061 | 1.00 | 14.08 | C |
| ATOM | 1248 | CD1 | ILE | A | 254 | −88.939 | 53.315 | 20.886 | 1.00 | 14.07 | C |
| ATOM | 1249 | CG2 | ILE | A | 254 | −91−181 | 55.942 | 22.998 | 1.00 | 14.27 | C |
| ATOM | 1250 | C | ILE | A | 254 | −89.942 | 56.071 | 25.780 | 1.00 | 13.92 | C |
| ATOM | 1251 | O | ILE | A | 254 | −89.692 | 54.907 | 26.106 | 1.00 | 13.59 | O |
| ATOM | 1252 | N | ILE | A | 255 | −90.736 | 56.879 | 26.488 | 1.00 | 13.92 | N |
| ATOM | 1253 | CA | ILE | A | 255 | −91.472 | 56.431 | 27.668 | 1.00 | 14.17 | C |
| ATOM | 1254 | CB | ILE | A | 255 | −91.508 | 57.501 | 28.768 | 1.00 | 14.18 | C |
| ATOM | 1255 | CG1 | ILE | A | 255 | −90.089 | 57.945 | 29.172 | 1.00 | 14.33 | C |
| ATOM | 1256 | CD1 | ILE | A | 255 | −89.165 | 56.823 | 29.614 | 1.00 | 14.26 | C |
| ATOM | 1257 | CG2 | ILE | A | 255 | −92.252 | 56.977 | 30.018 | 1.00 | 14.12 | C |
| ATOM | 1258 | C | ILE | A | 255 | −92.894 | 56.111 | 27.241 | 1.00 | 14.57 | C |
| ATOM | 1259 | O | ILE | A | 255 | −93.564 | 56.961 | 26.669 | 1.00 | 14.42 | O |
| ATOM | 1260 | N | THR | A | 256 | −93.345 | 54.891 | 27.511 | 1.00 | 15.20 | N |
| ATOM | 1261 | CA | THR | A | 256 | −94.722 | 54.496 | 27.215 | 1.00 | 15.99 | C |
| ATOM | 1262 | CB | THR | A | 256 | −94.786 | 53.320 | 26.224 | 1.00 | 16.05 | C |
| ATOM | 1263 | OG1 | THR | A | 256 | −93.920 | 52.276 | 26.661 | 1.00 | 16.39 | O |
| ATOM | 1264 | CG2 | THR | A | 256 | −94.356 | 53.756 | 24.843 | 1.00 | 16.10 | C |
| ATOM | 1265 | C | THR | A | 256 | −95.454 | 54.133 | 28.500 | 1.00 | 16.49 | C |
| ATOM | 1266 | O | THR | A | 256 | −94.861 | 53.603 | 29.437 | 1.00 | 16.93 | O |
| ATOM | 1267 | N | LEU | A | 257 | −96.741 | 54.457 | 28.534 | 1.00 | 17.21 | N |
| ATOM | 1268 | CA | LEU | A | 257 | −97.637 | 54.083 | 29.609 | 1.00 | 17.60 | C |
| ATOM | 1269 | CB | LEU | A | 257 | −98.464 | 55.301 | 30.033 | 1.00 | 18.13 | C |
| ATOM | 1270 | CG | LEU | A | 257 | −99.169 | 55.223 | 31.365 | 1.00 | 18.26 | C |
| ATOM | 1271 | CD1 | LEU | A | 257 | −96.181 | 55.466 | 32.511 | 1.00 | 18.50 | C |
| ATOM | 1272 | CD2 | LEU | A | 257 | −100.306 | 56.229 | 31.465 | 1.00 | 18.55 | C |
| ATOM | 1273 | C | LEU | A | 257 | −98.530 | 52.994 | 29.033 | 1.00 | 17.87 | C |
| ATOM | 1274 | O | LEU | A | 257 | −99.166 | 53.211 | 27.999 | 1.00 | 18.19 | O |
| ATOM | 1275 | N | GLU | A | 258 | −98.565 | 51.828 | 29.675 | 1.00 | 17.85 | N |
| ATOM | 1276 | CA | GLU | A | 258 | −99.359 | 50.690 | 29.192 | 1.00 | 18.16 | C |
| ATOM | 1277 | CB | GLU | A | 258 | −98.484 | 49.673 | 28.430 | 1.00 | 18.26 | C |
| ATOM | 1278 | CG | GLU | A | 258 | −96.973 | 49.827 | 28.622 | 1.00 | 18.42 | C |
| ATOM | 1279 | CD | GLU | A | 258 | −96.191 | 48.586 | 28.222 | 1.00 | 18.38 | C |
| ATOM | 1280 | OE1 | GLU | A | 258 | −96.323 | 47.554 | 28.908 | 1.00 | 18.75 | O |
| ATOM | 1281 | OE2 | GLU | A | 258 | −95.419 | 48.651 | 27.241 | 1.00 | 18.17 | O |
| ATOM | 1282 | C | GLU | A | 258 | −100.102 | 50.012 | 30.341 | 1.00 | 18.30 | C |
| ATOM | 1283 | O | GLU | A | 258 | −99.590 | 49.953 | 31.456 | 1.00 | 18.32 | O |
| ATOM | 1264 | N | ASP | A | 259 | −101.308 | 49.506 | 30.068 | 1.00 | 18.85 | N |
| ATOM | 1285 | CA | ASP | A | 259 | −102.068 | 48.747 | 31.074 | 1.00 | 19.18 | C |
| ATOM | 1266 | CB | ASP | A | 259 | −103.585 | 48.713 | 30.755 | 1.00 | 19.22 | C |
| ATOM | 1267 | CG | ASP | A | 259 | −103.959 | 47.804 | 29.581 | 1.00 | 19.22 | C |
| ATOM | 1288 | OD1 | ASP | A | 259 | −103.135 | 47.008 | 29.088 | 1.00 | 19.07 | O |
| ATOM | 1289 | OD2 | ASP | A | 259 | −105.128 | 47.889 | 29.144 | 1.00 | 19.50 | O |
| ATOM | 1290 | C | ASP | A | 259 | −101.462 | 47.351 | 31.258 | 1.00 | 19.45 | C |
| ATOM | 1291 | O | ASP | A | 259 | −100.533 | 46.976 | 30.534 | 1.00 | 18.62 | O |
| ATOM | 1292 | N | SER | A | 260 | −101.991 | 46.586 | 32.215 | 1.00 | 20.34 | N |
| ATOM | 1293 | CA | SER | A | 260 | −101.456 | 45.257 | 32.544 | 1.00 | 21.14 | C |
| ATOM | 1294 | CB | SER | A | 260 | −102.192 | 44.656 | 33.749 | 1.00 | 21.13 | C |
| ATOM | 1295 | OG | SER | A | 260 | −103.593 | 44.628 | 33.540 | 1.00 | 21.24 | O |
| ATOM | 1296 | C | SER | A | 260 | −101.463 | 44.263 | 31.372 | 1.00 | 21.97 | C |
| ATOM | 1297 | O | SER | A | 260 | −100.614 | 43.369 | 31.323 | 1.00 | 23.27 | O |
| ATOM | 1298 | N | SER | A | 261 | −102.396 | 44.435 | 30.433 | 1.00 | 22.35 | N |
| ATOM | 1299 | CA | SER | A | 261 | −102.465 | 43.610 | 29.217 | 1.00 | 22.27 | C |
| ATOM | 1300 | CB | SER | A | 261 | −103.910 | 43.567 | 28.700 | 1.00 | 22.60 | C |
| ATOM | 1301 | OG | SER | A | 261 | −104.782 | 43.023 | 29.671 | 1.00 | 23.01 | O |
| ATOM | 1302 | C | SER | A | 261 | −101.533 | 44.062 | 28.076 | 1.00 | 22.05 | C |
| ATOM | 1303 | O | SER | A | 261 | −101.546 | 43.459 | 26.999 | 1.00 | 22.14 | O |
| ATOM | 1304 | N | GLY | A | 262 | −100.739 | 45.113 | 28.297 | 1.00 | 21.65 | N |
| ATOM | 1305 | CA | GLY | A | 262 | −99.803 | 45.622 | 27.291 | 1.00 | 21.53 | C |
| ATOM | 1306 | C | GLY | A | 262 | −100.373 | 46.613 | 26.287 | 1.00 | 21.29 | C |
| ATOM | 1307 | O | GLY | A | 262 | −99.684 | 46.986 | 25.336 | 1.00 | 21.55 | O |
| ATOM | 1308 | N | ASN | A | 263 | −101.619 | 47.045 | 26.483 | 1.00 | 20.93 | N |
| ATOM | 1309 | CA | ASN | A | 263 | −102.240 | 48.037 | 25.601 | 1.00 | 20.49 | C |
| ATOM | 1310 | CB | ASN | A | 263 | −103.754 | 48.083 | 25.825 | 1.00 | 20.60 | C |
| ATOM | 1311 | CG | ASN | A | 263 | −104.449 | 46.816 | 25.362 | 1.00 | 20.66 | C |
| ATOM | 1312 | OD1 | ASN | A | 263 | −104.373 | 46.452 | 24.191 | 1.00 | 20.86 | O |
| ATOM | 1313 | ND2 | ASN | A | 263 | −105.135 | 46.143 | 26.278 | 1.00 | 21.05 | N |
| ATOM | 1314 | C | ASN | A | 263 | −101 .633 | 49.417 | 25.854 | 1.00 | 19.95 | C |
| ATOM | 1315 | O | ASN | A | 263 | −101.511 | 49.831 | 27.003 | 1.00 | 19.73 | O |
| ATOM | 1316 | N | LEU | A | 264 | −101.242 | 50.113 | 24.786 | 1.00 | 19.85 | N |
| ATOM | 1317 | CA | LEU | A | 264 | −100.637 | 51.442 | 24.912 | 1.00 | 19.81 | C |
| ATOM | 1318 | CB | LEU | A | 264 | −100.044 | 51.922 | 23.584 | 1.00 | 19.91 | C |
| ATOM | 1319 | CG | LEN | A | 264 | −99.211 | 53.215 | 23.701 | 1.00 | 20.07 | C |
| ATOM | 1320 | CD1 | LEU | A | 264 | −97.738 | 52.898 | 23.892 | 1.00 | 19.97 | C |
| ATOM | 1321 | CD2 | LEU | A | 264 | −99.412 | 54.127 | 22.498 | 1.00 | 20.28 | C |
| ATOM | 1322 | C | LEU | A | 264 | −101.675 | 52.455 | 25.393 | 1.00 | 19.71 | C |
| ATOM | 1323 | O | LEU | A | 264 | −102.744 | 52.589 | 24.786 | 1.00 | 19.97 | O |
| ATOM | 1324 | N | LEU | A | 265 | −101.350 | 53.146 | 26.485 | 1.00 | 18.92 | N |
| ATOM | 1325 | CA | LEU | A | 265 | −102.181 | 54.219 | 27.033 | 1.00 | 18.46 | C |
| ATOM | 1326 | CB | LEU | A | 265 | −102.294 | 54.087 | 28.558 | 1.00 | 18.22 | C |
| ATOM | 1327 | CG | LEU | A | 265 | −102.739 | 52.717 | 29.092 | 1.00 | 17.94 | C |
| ATOM | 1328 | CD1 | LEU | A | 265 | −102.911 | 52.764 | 30.600 | 1.00 | 17.71 | C |
| ATOM | 1329 | CD2 | LEU | A | 265 | −104.021 | 52.234 | 28.421 | 1.00 | 18.07 | C |
| ATOM | 1330 | C | LEU | A | 265 | −101.658 | 55.605 | 26.655 | 1.00 | 18.33 | C |
| ATOM | 1331 | O | LEU | A | 265 | −102.452 | 56.508 | 26.395 | 1.00 | 18.54 | O |
| ATOM | 1332 | N | GLY | A | 266 | −100.335 | 55.774 | 26.636 | 1.00 | 17.96 | N |
| ATOM | 1333 | CA | GLY | A | 266 | −99.713 | 57.028 | 26.199 | 1.00 | 17.79 | C |
| ATOM | 1334 | C | GLY | A | 266 | −98.238 | 56.868 | 25.868 | 1.00 | 17.55 | C |
| ATOM | 1335 | O | GLY | A | 266 | −97.636 | 55.848 | 25.206 | 1.00 | 18.06 | O |
| ATOM | 1336 | N | ARG | A | 267 | −97.669 | 57.869 | 25.194 | 1.00 | 17.17 | N |
| ATOM | 1337 | CA | ARG | A | 267 | −96.241 | 57.892 | 24.858 | 1.00 | 16.65 | C |
| ATOM | 1338 | CB | ARG | A | 267 | −95.993 | 57.270 | 23.473 | 1.00 | 16.99 | C |
| ATOM | 1339 | CG | ARG | A | 267 | −94.517 | 57.069 | 23.117 | 1.00 | 17.16 | C |
| ATOM | 1340 | CD | ARG | A | 267 | −94.289 | 56.772 | 21.644 | 1.00 | 17.30 | C |
| ATOM | 1341 | NE | ARG | A | 267 | −94.933 | 55.537 | 21.218 | 1.00 | 17.49 | N |
| ATOM | 1342 | CZ | ARG | A | 267 | −96.117 | 55.435 | 20.609 | 1.00 | 17.73 | C |
| ATOM | 1343 | NH1 | ARG | A | 267 | −96.864 | 56.506 | 20.319 | 1.00 | 17.77 | N |
| ATOM | 1344 | NH2 | ARG | A | 267 | −96.566 | 54.226 | 20.285 | 1.00 | 17.91 | N |
| ATOM | 1345 | C | ARG | A | 267 | −95.661 | 59.309 | 24.872 | 1.00 | 16.05 | C |
| ATOM | 1346 | O | ARG | A | 267 | −96.333 | 60.272 | 24.483 | 1.00 | 15.89 | O |
| ATOM | 1347 | N | AASN | A | 268 | −94.415 | 59.416 | 25.336 | 0.50 | 15.68 | N |
| ATOM | 1348 | N | BASN | A | 268 | −94.411 | 59.424 | 25.314 | 0.50 | 15.58 | N |
| ATOM | 1349 | CA | AASN | A | 268 | −93.617 | 60.636 | 25.236 | 0.50 | 15.52 | C |
| ATOM | 1350 | CA | BASN | A | 268 | −93.640 | 60.655 | 25.181 | 0.50 | 15.36 | C |
| ATOM | 1351 | CB | AASN | A | 268 | −93.699 | 61.444 | 26.529 | 0.50 | 15.50 | C |
| ATOM | 1352 | CB | BASN | A | 268 | −93.818 | 61.538 | 26.419 | 0.50 | 15.20 | C |
| ATOM | 1353 | CG | AASN | A | 268 | −94.826 | 62.447 | 26.518 | 0.50 | 15.63 | C |
| ATOM | 1354 | CG | BASN | A | 268 | −93.659 | 63.021 | 26.119 | 0.50 | 15.23 | C |
| ATOM | 1355 | OD1 | AASN | A | 268 | −94.889 | 63.328 | 25.655 | 0.50 | 15.61 | O |
| ATOM | 1356 | OD1 | BASN | A | 268 | −92.655 | 63.447 | 25.547 | 0.50 | 14.93 | O |
| ATOM | 1357 | ND2 | AASN | A | 268 | −95.725 | 62.322 | 27.485 | 0.50 | 15.63 | N |
| ATOM | 1358 | ND2 | BASN | A | 268 | −94.652 | 63.326 | 26.512 | 0.50 | 15.19 | N |
| ATOM | 1359 | C | AASN | A | 268 | −92.178 | 63.816 | 24.970 | 0.50 | 15.28 | C |
| ATOM | 1360 | C | BASN | A | 268 | −92.184 | 60.228 | 24.990 | 0.50 | 15.20 | C |
| ATOM | 1361 | O | AASN | A | 268 | −91.863 | 60.241 | 24.960 | 0.50 | 15.21 | O |
| ATOM | 1362 | O | BASN | A | 268 | −91.866 | 59.050 | 25.047 | 0.50 | 15.17 | O |
| ATOM | 1363 | N | SER | A | 269 | −91.310 | 61.210 | 24.738 | 1.00 | 15.09 | N |
| ATOM | 1364 | CA | SER | A | 269 | −89.893 | 60.937 | 24.548 | 1.00 | 15.14 | C |
| ATOM | 1365 | CB | SER | A | 269 | −89.633 | 60.362 | 23.149 | 1.00 | 15.35 | C |
| ATOM | 1366 | OG | SER | A | 269 | −90.017 | 61.285 | 22.151 | 1.00 | 15.70 | O |
| ATOM | 1367 | C | SER | A | 269 | −89.013 | 62.148 | 24.756 | 1.00 | 14.87 | C |
| ATOM | 1368 | O | SER | A | 269 | −89.468 | 63.294 | 24.731 | 1.00 | 14.71 | O |
| ATOM | 1369 | N | PHE | A | 270 | −87.736 | 61.863 | 24.960 | 1.00 | 14.71 | N |
| ATOM | 1370 | CA | PHE | A | 270 | −86.724 | 62.887 | 25.095 | 1.00 | 14.56 | C |
| ATOM | 1371 | CB | PHE | A | 270 | −86.689 | 63.432 | 26.524 | 1.00 | 14.32 | C |
| ATOM | 1372 | CG | PHE | A | 270 | −86.431 | 62.390 | 27.579 | 1.00 | 14.23 | C |
| ATOM | 1373 | CD1 | PHE | A | 270 | −87.484 | 61.648 | 28.119 | 1.00 | 14.16 | C |
| ATOM | 1374 | OE1 | PHE | A | 270 | −87.251 | 60.696 | 29.100 | 1.00 | 14.04 | C |
| ATOM | 1375 | CZ | PHE | A | 270 | −85.962 | 60.476 | 29.560 | 1.00 | 13.98 | C |
| ATOM | 1376 | CE2 | PHE | A | 270 | −84.911 | 61.209 | 29.037 | 1.00 | 14.10 | C |
| ATOM | 1377 | CD2 | PHE | A | 270 | −85.144 | 62.168 | 28.055 | 1.00 | 14.10 | C |
| ATOM | 1378 | C | PHE | A | 270 | −85.375 | 62.312 | 24.704 | 1.00 | 14.45 | C |
| ATOM | 1379 | O | PHE | A | 270 | −85.112 | 61.127 | 24.905 | 1.00 | 14.39 | O |
| ATOM | 1380 | N | GLU | A | 271 | −84.533 | 63.167 | 24.141 | 1.00 | 14.90 | N |
| ATOM | 1381 | CA | GLU | A | 271 | −83.182 | 62.797 | 23.744 | 1.00 | 15.13 | C |
| ATOM | 1382 | CB | GLU | A | 271 | −82.618 | 63.864 | 22.807 | 1.00 | 15.44 | C |
| ATOM | 1383 | CG | GLU | A | 271 | −81.342 | 63.476 | 22.083 | 1.00 | 15.89 | C |
| ATOM | 1384 | CD | GLU | A | 271 | −81.013 | 64.410 | 20.932 | 1.00 | 16.14 | C |
| ATOM | 1385 | OE1 | GLU | A | 271 | −81.912 | 64.703 | 20.110 | 1.00 | 17.03 | O |
| ATOM | 1386 | OE2 | GLU | A | 271 | −79.847 | 64.842 | 20.837 | 1.00 | 15.39 | O |
| ATOM | 1387 | C | GLU | A | 271 | −82.347 | 62.572 | 25.013 | 1.00 | 15.17 | C |
| ATOM | 1388 | O | GLU | A | 271 | −82.677 | 63.282 | 26.023 | 1.00 | 14.60 | O |
| ATOM | 1389 | N | VAL | A | 272 | −81.290 | 61.862 | 24.979 | 1.00 | 15.56 | N |
| ATOM | 1390 | CA | VAL | A | 272 | −80.410 | 61.712 | 26.144 | 1.00 | 15.97 | C |
| ATOM | 1391 | CB | VAL | A | 272 | −80.735 | 60.454 | 25.986 | 1.00 | 16.23 | C |
| ATOM | 1392 | CG1 | VAL | A | 272 | −79.972 | 60.477 | 28.313 | 1.00 | 16.25 | C |
| ATOM | 1393 | CG2 | VAL | A | 272 | −82.225 | 60.341 | 27.241 | 1.00 | 16.44 | C |
| ATOM | 1394 | C | VAL | A | 272 | −78.960 | 61.518 | 25.711 | 1.00 | 15.35 | C |
| ATOM | 1395 | O | VAL | A | 272 | −78.653 | 60.988 | 24.705 | 1.00 | 15.94 | O |
| ATOM | 1396 | N | ARG | A | 273 | −78.081 | 62.245 | 26.490 | 1.00 | 17.37 | N |
| ATOM | 1397 | CA | ARG | A | 273 | −76.639 | 62.122 | 26.321 | 1.00 | 18.15 | C |
| ATOM | 1398 | CB | ARG | A | 273 | −76.065 | 63.426 | 25.755 | 1.00 | 19.16 | C |
| ATOM | 1399 | CG | ARG | A | 273 | −74.551 | 63.473 | 25.542 | 1.00 | 20.20 | C |
| ATOM | 1400 | CD | ARG | A | 273 | −74.079 | 64.808 | 25.083 | 1.00 | 20.98 | C |
| ATOM | 1401 | NE | ARG | A | 273 | −72.623 | 64.924 | 25.147 | 1.00 | 21.82 | N |
| ATOM | 1402 | CZ | ARG | A | 273 | −71.764 | 64.308 | 24.332 | 1.00 | 22.51 | C |
| ATOM | 1403 | NH1 | ARG | A | 273 | −72.189 | 63.506 | 23.353 | 1.00 | 22.88 | N |
| ATOM | 1404 | NH2 | ARG | A | 273 | −70.454 | 64.492 | 24.500 | 1.00 | 22.89 | N |
| ATOM | 1405 | C | ARG | A | 273 | −76.028 | 61.797 | 27.682 | 1.00 | 18.14 | C |
| ATOM | 1406 | O | ARG | A | 273 | −76.214 | 62.541 | 28.542 | 1.00 | 17.96 | O |
| ATOM | 1407 | N | VAL | A | 274 | −75.352 | 60.655 | 27.767 | 1.00 | 18.25 | N |
| ATOM | 1408 | CA | VAL | A | 274 | −74.514 | 60.315 | 28.912 | 1.00 | 18.37 | C |
| ATOM | 1409 | CB | VAL | A | 274 | −74.470 | 58.788 | 29.152 | 1.00 | 18.44 | C |
| ATOM | 1410 | CG1 | VAL | A | 274 | −73.406 | 58.407 | 30.180 | 1.00 | 18.51 | C |
| ATOM | 1411 | CG2 | VAL | A | 274 | −75.838 | 58.313 | 29.606 | 1.00 | 18.50 | C |
| ATOM | 1412 | C | VAL | A | 274 | −73.120 | 60.885 | 28.680 | 1.00 | 18.33 | C |
| ATOM | 1413 | O | VAL | A | 274 | −72.493 | 60.606 | 27.660 | 1.00 | 18.36 | O |
| ATOM | 1414 | N | VAL | A | 275 | −72.644 | 61.692 | 29.625 | 1.00 | 18.50 | N |
| ATOM | 1415 | CA | CYS | A | 275 | −71.352 | 62.366 | 29.482 | 1.00 | 18.30 | C |
| ATOM | 1416 | CB | CYS | A | 275 | −71.500 | 63.602 | 28.591 | 1.00 | 18.03 | C |
| ATOM | 1417 | SG | CYS | A | 275 | −72.756 | 64.770 | 29.162 | 1.00 | 17.50 | S |
| ATOM | 1418 | C | CYS | A | 275 | −70.752 | 62.751 | 30.826 | 1.00 | 18.55 | C |
| ATOM | 1419 | O | CYS | A | 275 | −71.462 | 62.874 | 31.830 | 1.00 | 18.75 | O |
| ATOM | 1420 | N | ALA | A | 276 | −69.436 | 62.939 | 30.834 | 1.00 | 18.35 | N |
| ATOM | 1421 | CA | ALA | A | 276 | −68.703 | 63.265 | 32.058 | 1.00 | 18.39 | C |
| ATOM | 1422 | CB | ALA | A | 276 | −67.201 | 63.223 | 31.804 | 1.00 | 18.37 | C |
| ATOM | 1423 | C | ALA | A | 276 | −69.095 | 64.613 | 32.660 | 1.00 | 18.36 | C |
| ATOM | 1424 | O | ALA | A | 276 | −69.150 | 64.745 | 33.886 | 1.00 | 18.03 | O |
| ATOM | 1425 | N | CYS | A | 277 | −69.384 | 65.591 | 31.799 | 1.00 | 18.49 | N |
| ATOM | 1426 | CA | CYS | A | 277 | −69.703 | 66.958 | 32.215 | 1.00 | 18.64 | C |
| ATOM | 1427 | CB | CYS | A | 277 | −68.751 | 67.900 | 31.793 | 1.00 | 19.02 | C |
| ATOM | 1428 | SG | CYS | A | 277 | −66.948 | 67.467 | 32.475 | 1.00 | 20.15 | S |
| ATOM | 1429 | C | CYS | A | 277 | −71_039 | 67.428 | 31.611 | 1.00 | 18.25 | C |
| ATOM | 1430 | O | CYS | A | 277 | −71−040 | 68.219 | 30.666 | 1.00 | 17.68 | O |
| ATOM | 1431 | N | PRO | A | 278 | −72.181 | 66.961 | 32.169 | 1.00 | 18.12 | N |
| ATOM | 1432 | CA | PRO | A | 278 | −73.514 | 67.322 | 31.638 | 1.00 | 18.02 | C |
| ATOM | 1433 | CB | PRO | A | 278 | −74.480 | 66.720 | 32.664 | 1.00 | 18.10 | C |
| ATOM | 1434 | CG | PRO | A | 278 | −73.711 | 65.640 | 33.337 | 1.00 | 18.17 | C |
| ATOM | 1435 | CD | PRO | A | 278 | −72.292 | 66.118 | 33.375 | 1.00 | 18.21 | C |
| ATOM | 1436 | C | PRO | A | 278 | −73.764 | 68.830 | 31.496 | 1.00 | 17.82 | C |
| ATOM | 1437 | O | PRO | A | 278 | −74.233 | 69.277 | 30.445 | 1.00 | 17.71 | O |
| ATOM | 1438 | N | GLY | A | 279 | −73.450 | 69.591 | 32.542 | 1.00 | 17.77 | N |
| ATOM | 1439 | CA | GLY | A | 279 | −73.617 | 71.047 | 32.530 | 1.00 | 17.83 | C |
| ATOM | 1440 | C | GLY | A | 279 | −72.904 | 71.720 | 31.367 | 1.00 | 18.05 | C |
| ATOM | 1441 | O | GLY | A | 279 | −73.508 | 72.500 | 30.631 | 1.00 | 17.93 | O |
| ATOM | 1442 | N | ARG | A | 280 | −71.623 | 71.396 | 31.194 | 1.00 | 18.05 | N |
| ATOM | 1443 | C | ARG | A | 280 | −70.813 | 71.942 | 30.097 | 1.00 | 18.73 | C |
| ATOM | 1444 | CB | ARG | A | 280 | −69.354 | 71.470 | 30.218 | 1.00 | 19.52 | C |
| ATOM | 1445 | CG | ARG | A | 280 | −68.431 | 71.974 | 29.111 | 1.00 | 20.36 | C |
| ATOM | 1446 | CD | ARG | A | 280 | −66.916 | 71.650 | 29.410 | 1.00 | 21.23 | C |
| ATOM | 1447 | NE | ARG | A | 280 | −66.738 | 70.206 | 29.420 | 1.00 | 22.23 | N |
| ATOM | 1448 | CZ | ARG | A | 280 | −65.624 | 69.606 | 29.843 | 1.00 | 22.68 | C |
| ATOM | 1449 | NH1 | ARG | A | 280 | −64.594 | 70.304 | 30.322 | 1.00 | 23.10 | N |
| ATOM | 1450 | NH2 | ARG | A | 280 | −65.543 | 68.278 | 29.798 | 1.00 | 23.18 | N |
| ATOM | 1451 | C | ARG | A | 280 | −71.364 | 71.567 | 28.720 | 1.00 | 18.32 | C |
| ATOM | 1452 | O | ARG | A | 280 | −71.479 | 72.423 | 27.834 | 1.00 | 18.43 | O |
| ATOM | 1453 | N | ASP | A | 281 | −71_695 | 70.291 | 28.544 | 1.00 | 17.62 | N |
| ATOM | 1454 | CA | ASP | A | 281 | −72.242 | 69.810 | 27.276 | 1.00 | 17.36 | C |
| ATOM | 1455 | CB | ASP | A | 281 | −72.265 | 68.273 | 27.227 | 1.00 | 17.64 | C |
| ATOM | 1456 | CG | ASP | A | 281 | −70.868 | 67.663 | 27.106 | 1.00 | 18.17 | C |
| ATOM | 1457 | OD1 | ASP | A | 281 | −69.896 | 68.391 | 26.800 | 1.00 | 18.42 | O |
| ATOM | 1458 | OD2 | ASP | A | 281 | −70.742 | 66.436 | 27.307 | 1.00 | 18.60 | O |
| ATOM | 1459 | C | ASP | A | 281 | −73.623 | 70.393 | 26.959 | 1.00 | 16.51 | C |
| ATOM | 1460 | O | ASP | A | 281 | −73.900 | 70.675 | 25.798 | 1.00 | 16.16 | O |
| ATOM | 1461 | N | ARG | A | 282 | −74.476 | 70.579 | 27.969 | 1.00 | 16.28 | N |
| ATOM | 1462 | CA | ARG | A | 282 | −75.751 | 71.288 | 27.762 | 1.00 | 16.22 | C |
| ATOM | 1463 | CB | ARG | A | 282 | −76.663 | 71.250 | 29.001 | 1.00 | 16.16 | C |
| ATOM | 1464 | CG | ARG | A | 282 | −77.967 | 72.020 | 28.783 | 1.00 | 16.00 | C |
| ATOM | 1465 | CD | ARG | A | 282 | −78.933 | 71.999 | 29.948 | 1.00 | 15.93 | C |
| ATOM | 1466 | NE | ARG | A | 282 | −79.892 | 73.108 | 29.825 | 1.00 | 15.87 | N |
| ATOM | 1467 | CZ | ARG | A | 282 | −80.590 | 73.653 | 30.825 | 1.00 | 16.11 | C |
| ATOM | 1468 | NH1 | ARG | A | 282 | −80.477 | 73.211 | 32.083 | 1.00 | 16.22 | N |
| ATOM | 1469 | NH2 | ARG | A | 282 | −81.423 | 74.661 | 30.562 | 1.00 | 16.09 | N |
| ATOM | 1470 | C | ARG | A | 282 | −75.508 | 72.739 | 27.330 | 1.00 | 16.31 | C |
| ATOM | 1471 | O | ARG | A | 282 | −76.186 | 73.234 | 24.436 | 1.00 | 15.27 | O |
| ATOM | 1472 | N | ARG | A | 283 | −74.559 | 73.415 | 27.977 | 1.00 | 17.10 | N |
| ATOM | 1473 | CA | ARG | A | 283 | −74.194 | 74.789 | 27.589 | 1.00 | 17.86 | C |
| ATOM | 1474 | CB | ARG | A | 283 | −73.160 | 75.384 | 28.562 | 1.00 | 17.91 | C |
| ATOM | 1475 | CG | ARG | A | 283 | −73.721 | 75.805 | 29.907 | 1.00 | 18.05 | C |
| ATOM | 1476 | CD | ARG | A | 283 | −72.655 | 76.474 | 30.768 | 1.00 | 18.10 | C |
| ATOM | 1477 | NE | ARG | A | 283 | −73.034 | 76.473 | 32.179 | 1.00 | 18.15 | N |
| ATOM | 1478 | CZ | ARG | A | 283 | −72.247 | 76.846 | 33.191 | 1.00 | 18.62 | C |
| ATOM | 1479 | NH1 | ARG | A | 283 | −72.175 | 76.787 | 34.434 | 1.00 | 18.74 | N |
| ATOM | 1480 | NH2 | ARG | A | 283 | −71.005 | 77.283 | 32.980 | 1.00 | 18.86 | N |
| ATOM | 1481 | C | ARG | A | 283 | −73.669 | 74.866 | 26.154 | 1.00 | 18.59 | C |
| ATOM | 1482 | O | ARG | A | 283 | −73.977 | 75.812 | 25.434 | 1.00 | 18.94 | O |
| ATOM | 1483 | N | THR | A | 283 | −73.905 | 73.855 | 25.743 | 1.00 | 19.42 | N |
| ATOM | 1484 | CA | THR | A | 283 | −72.293 | 73.827 | 24.413 | 1.00 | 19.98 | C |
| ATOM | 1485 | CB | THR | A | 283 | −71.144 | 72.805 | 24.357 | 1.00 | 19.84 | C |
| ATOM | 1486 | OG1 | THR | A | 283 | −70.303 | 72.986 | 225.503 | 1.00 | 19.68 | O |
| ATOM | 1487 | CG2 | THR | A | 283 | −70.302 | 72.992 | 23.097 | 1.00 | 19.97 | C |
| ATOM | 1488 | C | THR | A | 283 | −73.317 | 73.560 | 23.302 | 1.00 | 20.82 | C |
| ATOM | 1489 | O | THR | A | 284 | −73.316 | 74.246 | 22.282 | 1.00 | 20.13 | O |
| ATOM | 1490 | N | GLU | A | 285 | −74.193 | 72.580 | 23.503 | 1.00 | 21.78 | N |
| ATOM | 1491 | CA | GLU | A | 285 | −75.215 | 72.257 | 22.496 | 1.00 | 22.99 | C |
| ATOM | 1492 | CB | GLU | A | 285 | −75.897 | 70.922 | 22.812 | 1.00 | 23.04 | C |
| ATOM | 1493 | CG | GLU | A | 285 | −74.944 | 69.739 | 22.687 | 1.00 | 23.16 | C |
| ATOM | 1494 | CD | GLU | A | 285 | −75.632 | 68.387 | 22.674 | 1.00 | 23.13 | C |
| ATOM | 1495 | OE1 | GLU | A | 285 | −76.813 | 68.301 | 22.273 | 1.00 | 23.74 | O |
| ATOM | 1496 | OE2 | GLU | A | 285 | −74.973 | 67.397 | 23.052 | 1.00 | 22.79 | O |
| ATOM | 1497 | C | GLU | A | 285 | −76.252 | 73.368 | 22.325 | 1.00 | 23.72 | C |
| ATOM | 1498 | O | GLU | A | 285 | −76.729 | 73.60S | 21.214 | 1.00 | 23.80 | O |
| ATOM | 1499 | N | GLU | A | 286 | −76.586 | 74.052 | 23.416 | 1.00 | 24.78 | N |
| ATOM | 1500 | CA | GLU | A | 286 | −77.533 | 75.170 | 23.360 | 1.00 | 26.41 | C |
| ATOM | 1501 | CB | GLU | A | 286 | −78.021 | 75.542 | 24.763 | 1.00 | 26.14 | C |
| ATOM | 1502 | CG | GLU | A | 286 | −78.980 | 74.512 | 25.344 | 1.00 | 26.18 | C |
| ATOM | 1503 | CD | GLU | A | 286 | −79.510 | 74.885 | 26.714 | 1.00 | 26.30 | C |
| ATOM | 1504 | OE1 | GLU | A | 286 | −79.105 | 75.934 | 27.264 | 1.00 | 26.64 | O |
| ATOM | 1505 | OE2 | GLU | A | 286 | −80.345 | 74.122 | 27.242 | 1.00 | 26.01 | O |
| ATOM | 1506 | C | GLU | A | 286 | −76.990 | 76.405 | 22.623 | 1.00 | 27.63 | C |
| ATOM | 1507 | O | GLU | A | 286 | −77.764 | 77.102 | 21.967 | 1.00 | 27.24 | O |
| ATOM | 1508 | N | GLU | A | 287 | −75.682 | 76.669 | 22.716 | 1.00 | 29.62 | N |
| ATOM | 1509 | CA | GLU | A | 287 | −75.071 | 77.788 | 21.964 | 1.00 | 31.48 | C |
| ATOM | 1510 | CB | GLU | A | 287 | −73.681 | 78.183 | 22.513 | 1.00 | 31.53 | C |
| ATOM | 1511 | CG | GLU | A | 287 | −72.554 | 77.168 | 22.359 | 1.00 | 31.30 | C |
| ATOM | 1512 | CD | GLU | A | 287 | −71.173 | 77.730 | 22.687 | 1.00 | 31.36 | C |
| ATOM | 1513 | OE1 | GLU | A | 287 | −70.350 | 76.983 | 23.259 | 1.00 | 30.38 | O |
| ATOM | 1514 | OE2 | GLU | A | 287 | −70.900 | 78.913 | 22.381 | 1.00 | 31.84 | O |
| ATOM | 1515 | C | GLU | A | 287 | −75.020 | 77.557 | 20.442 | 1.00 | 33.83 | C |
| ATOM | 1516 | O | GLU | A | 287 | −75.051 | 78.519 | 19.674 | 1.00 | 34.83 | O |
| ATOM | 1517 | N | ASN | A | 288 | −74.965 | 76.294 | 20.016 | 1.00 | 36.59 | N |
| ATOM | 1518 | CA | ASN | A | 288 | −75.017 | 75.943 | 18.585 | 1.00 | 38.59 | C |
| ATOM | 1519 | CB | ASN | A | 288 | −74.488 | 74.521 | 18.358 | 1.00 | 38.76 | C |
| ATOM | 1520 | CG | ASN | A | 288 | −73.035 | 74.359 | 18.760 | 1.00 | 39.15 | C |
| ATOM | 1521 | OD1 | ASN | A | 288 | −72.299 | 75.338 | 18.913 | 1.00 | 39.68 | O |
| ATOM | 1522 | ND2 | ASN | A | 288 | −72.612 | 73.112 | 18.934 | 1.00 | 38.95 | N |
| ATOM | 1523 | C | ASN | A | 288 | −76.415 | 76.052 | 17.960 | 1.00 | 40.45 | C |
| ATOM | 1524 | O | ASN | A | 288 | −76.534 | 76.047 | 16.737 | 1.00 | 40.69 | O |
| ATOM | 1525 | N | LEU | A | 289 | −77.458 | 76.139 | 18.788 | 1.00 | 43.13 | N |
| ATOM | 1526 | CA | LEU | A | 289 | −78.850 | 76.149 | 18.310 | 1.00 | 45.17 | C |
| ATOM | 1527 | CB | LEU | A | 289 | −79.823 | 75.924 | 19.487 | 1.00 | 44.97 | C |
| ATOM | 1528 | CG | LEU | A | 289 | −81.114 | 75.139 | 19.230 | 1.00 | 44.92 | C |
| ATOM | 1529 | CD1 | LEU | A | 289 | −80.821 | 73.722 | 18.752 | 1.00 | 44.79 | C |
| ATOM | 1530 | CD2 | LEU | A | 289 | −81.958 | 75.093 | 20.496 | 1.00 | 44.90 | C |
| ATOM | 1531 | C | LEU | A | 289 | −79.203 | 77.439 | 17.550 | 1.00 | 47.06 | C |
| ATOM | 1532 | O | LEU | A | 289 | −80.004 | 77.409 | 16.612 | 1.00 | 47.68 | O |
| ATOM | 1533 | N | ARG | A | 290 | −78.591 | 78.553 | 17.956 | 1.00 | 49.56 | N |
| ATOM | 1534 | CA | ARG | A | 290 | −78.765 | 79.867 | 17.293 | 1.00 | 51.33 | C |
| ATOM | 1535 | CB | ARG | A | 290 | −79.083 | 81.009 | 18.292 | 1.00 | 51.69 | C |
| ATOM | 1536 | CG | ARG | A | 290 | −78.627 | 80.824 | 19.741 | 1.00 | 51.68 | C |
| ATOM | 1537 | CD | ARG | A | 290 | −78.334 | 82.141 | 20.444 | 1.00 | 51.55 | C |
| ATOM | 1538 | NE | ARG | A | 290 | −79.427 | 83.113 | 20.392 | 1.00 | 51.43 | N |
| ATOM | 1539 | CZ | ARG | A | 290 | −79.408 | 847.302 | 21.000 | 1.00 | 51.04 | C |
| ATOM | 1540 | NH1 | ARG | A | 290 | −78.359 | 84.686 | 21.731 | 1.00 | 51.50 | N |
| ATOM | 1541 | NH2 | ARG | A | 290 | −81.452 | 85.118 | 20.884 | 1.00 | 51.52 | N |
| ATOM | 1542 | C | ARG | A | 290 | −77.567 | 80.247 | 16.405 | 1.00 | 53.16 | C |
| ATOM | 1543 | O | ARG | A | 290 | −80.849 | 15.344 | 15.344 | 1.00 | 53.45 | O |
| ATOM | 1544 | N | LYS | A | 291 | −76.351 | 79.905 | 16.842 | 1.00 | 54.52 | N |
| ATOM | 1545 | CA | LYS | A | 291 | −75.116 | 80.176 | 16.081 | 1.00 | 55.31 | C |
| ATOM | 1546 | CB | LYS | A | 291 | −73.890 | 79.690 | 16.870 | 1.00 | 55.63 | C |
| ATOM | 1547 | CG | LYS | A | 291 | −72.546 | 80.196 | 16.366 | 1.00 | 55.82 | C |
| ATOM | 1548 | CD | LYS | A | 291 | −71.404 | 79.572 | 17.158 | 1.00 | 56.00 | C |
| ATOM | 1549 | CE | LYS | A | 291 | −70.051 | 80.158 | 16.780 | 1.00 | 55.67 | C |
| ATOM | 1550 | NZ | LYS | A | 291 | −69.780 | 81.459 | 17.452 | 1.00 | 55.36 | N |
| ATOM | 1551 | C | LYS | A | 291 | −75.131 | 79.545 | 14.679 | 1.00 | 56.29 | C |
| ATOM | 1552 | O | LYS | A | 291 | −74.635 | 80.147 | 13.723 | 1.00 | 56.31 | O |
| ATOM | 1553 | N | LYS | A | 292 | −75.689 | 78.337 | 14.574 | 1.00 | 56.88 | N |
| ATOM | 1554 | CA | LYS | A | 292 | −75.950 | 77.687 | 13.282 | 1.00 | 57.00 | C |
| ATOM | 1555 | CB | LYS | A | 292 | −74.718 | 76.907 | 12.797 | 1.00 | 57.30 | C |
| ATOM | 1556 | CG | LYS | A | 292 | −73.688 | 77.745 | 12.047 | 1.00 | 57.29 | C |
| ATOM | 1557 | CD | LYS | A | 292 | −72.919 | 76.929 | 11.016 | 1.00 | 57.68 | C |
| ATOM | 1558 | CE | LYS | A | 292 | −73.721 | 76.729 | 9.736 | 1.00 | 57.70 | C |
| ATOM | 1559 | NZ | LYS | A | 292 | −72.954 | 75.977 | 8.705 | 1.00 | 57.56 | N |
| ATOM | 1560 | C | LYS | A | 292 | −77.161 | 76.756 | 13.374 | 1.00 | 57.04 | C |
| ATOM | 1561 | O | LYS | A | 292 | −77.062 | 75.629 | 13.860 | 1.00 | 56.87 | O |
| TER | 1562 | | LYS | A | 292 | | | | | | |
| ATOM | 1563 | N | VAL | B | 97 | −82.117 | 46.630 | 2.393 | 1.00 | 32.99 | N |
| ATOM | 1564 | CA | VAL | B | 97 | −81.308 | 45.372 | 2.344 | 1.00 | 33.37 | C |
| ATOM | 1565 | CB | VAL | B | 97 | −82.031 | 44.186 | 3.029 | 1.00 | 33.08 | C |
| ATOM | 1566 | CG1 | VAL | B | 97 | −81−096 | 42.989 | 3.171 | 1.00 | 33.07 | C |
| ATOM | 1567 | CG2 | VAL | B | 97 | −82.567 | 44.594 | 4.397 | 1.00 | 33.20 | C |
| ATOM | 1568 | C | VAL | B | 97 | −80.991 | 45.005 | 0.886 | 1.00 | 33.46 | C |
| ATOM | 1569 | O | VAL | B | 97 | −81.914 | 44.753 | 0.107 | 1.00 | 34.15 | O |
| ATOM | 1570 | N | PRO | B | 98 | −79.692 | 44.976 | 0.511 | 1.00 | 33.59 | N |
| ATOM | 1571 | CA | PRO | B | 98 | −79.326 | 44.566 | −0.846 | 1.00 | 33.44 | C |
| ATOM | 1572 | CB | PRO | B | 98 | −77.875 | 45.035 | −0.983 | 1.00 | 33.59 | C |
| ATOM | 1573 | CG | PRO | B | 98 | −77.343 | 45.078 | 0.404 | 1.00 | 33.75 | C |
| ATOM | 1574 | CD | PRO | B | 98 | −78.504 | 45.258 | 1.338 | 1.00 | 33.84 | C |
| ATOM | 1575 | C | PRO | B | 98 | −79.445 | 43.054 | −1.014 | 1.00 | 32.98 | C |
| ATOM | 1576 | O | PRO | B | 98 | −79.315 | 42.310 | −0.038 | 1.00 | 33.20 | O |
| ATOM | 1577 | N | SER | B | 99 | −79.694 | 42.620 | −2.246 | 1.00 | 33.05 | N |
| ATOM | 1578 | CA | SER | B | 99 | −80.004 | 41.222 | −2.538 | 1.00 | 33.06 | C |
| ATOM | 1579 | CB | SER | B | 99 | −80.488 | 41.074 | −3.983 | 1.00 | 33.19 | C |
| ATOM | 1580 | OG | SER | B | 99 | −80.881 | 39.740 | −4.252 | 1.00 | 33.64 | O |
| ATOM | 1581 | C | SER | B | 99 | −78.813 | 40.297 | −2.306 | 1.00 | 32.95 | C |
| ATOM | 1582 | O | SER | B | 99 | −77.669 | 40.665 | −2.580 | 1.00 | 32.43 | O |
| ATOM | 1583 | N | GLN | B | 100 | −79.109 | 39.101 | −1.796 | 1.00 | 33.27 | N |
| ATOM | 1584 | CA | GLN | B | 100 | −78.125 | 38.033 | −1.602 | 1.00 | 33.77 | C |
| ATOM | 1585 | CB | GLN | B | 100 | −78.070 | 37.639 | −0.118 | 1.00 | 34.47 | C |
| ATOM | 1586 | CG | GLN | B | 100 | −79.262 | 36.830 | 0.385 | 1.00 | 34.63 | C |
| ATOM | 1587 | CD | GLN | B | 100 | −79.492 | 36.993 | 1.874 | 1.00 | 34.92 | C |
| ATOM | 1588 | OE1 | GLN | B | 100 | −79.979 | 38.032 | 2.326 | 1.00 | 34.94 | O |
| ATOM | 1589 | NE2 | GLN | B | 100 | −79.152 | 35.964 | 2.644 | 1.00 | 34.36 | N |
| ATOM | 1590 | C | GLN | B | 100 | −78.442 | 36.804 | −2.468 | 1.00 | 34.18 | C |
| ATOM | 1591 | O | GLN | B | 100 | −77.915 | 35.718 | −2.215 | 1.00 | 33.76 | O |
| ATOM | 1592 | N | LYS | B | 101 | −79.292 | 36.978 | −3.485 | 1.00 | 34.63 | N |
| ATOM | 1593 | CA | LYS | B | 101 | −79.709 | 35.870 | −4.347 | 1.00 | 35.39 | C |
| ATOM | 1594 | CB | LYS | B | 101 | −81.005 | 36.210 | −5.109 | 1.00 | 35.90 | C |
| ATOM | 1595 | CG | LYS | B | 101 | −81.478 | 35.168 | −6.130 | 1.00 | 36.44 | C |
| ATOM | 1596 | CD | LYS | B | 101 | −81.713 | 33.786 | −5.523 | 1.00 | 37.32 | C |
| ATOM | 1597 | CE | LYS | B | 101 | −81.920 | 32.716 | −6.586 | 1.00 | 37.39 | C |
| ATOM | 1598 | NZ | LYS | B | 101 | −83.216 | 32.857 | −7.308 | 1.00 | 37.54 | N |
| ATOM | 1599 | C | LYS | B | 101 | −78.595 | 35.541 | −5.328 | 1.00 | 35.04 | C |
| ATOM | 1600 | O | LYS | B | 101 | −78.083 | 36.426 | −6.009 | 1.00 | 34.03 | O |
| ATOM | 1601 | N | THR | B | 102 | −78.234 | 34.234 | −5.400 | 1.00 | 35.64 | N |
| ATOM | 1602 | CA | THR | B | 102 | −77.241 | 33.789 | −6.356 | 1.00 | 35.57 | C |
| ATOM | 1603 | CB | THR | B | 102 | −76.925 | 32.296 | −6.137 | 1.00 | 35.40 | C |
| ATOM | 1604 | OG1 | THR | B | 102 | −76.436 | 32.108 | −4.803 | 1.00 | 35.17 | O |
| ATOM | 1605 | CG2 | THR | B | 102 | −75.876 | 31.797 | −7.132 | 1.00 | 34.85 | C |
| ATOM | 1606 | C | THR | B | 102 | −77.744 | 34.026 | −7.782 | 1.00 | 36.08 | C |
| ATOM | 1607 | O | THR | B | 102 | −78.878 | 33.684 | −8.116 | 1.00 | 36.44 | O |
| ATOM | 1608 | N | TYR | B | 103 | −76.889 | 34.633 | −8.600 | 1.00 | 36.65 | N |
| ATOM | 1609 | CA | TYR | B | 103 | −77.220 | 35.009 | −9.970 | 1.00 | 37.02 | C |
| ATOM | 1610 | CB | TYR | B | 103 | −77.775 | 36.441 | −9.986 | 1.00 | 37.05 | C |
| ATOM | 1611 | CG | TYR | B | 103 | −77.833 | 37.107 | −11.346 | 1.00 | 37.37 | C |
| ATOM | 1612 | CD1 | TYR | B | 103 | −76.614 | 36.576 | −12.375 | 1.00 | 37.66 | C |
| ATOM | 1613 | CE1 | TYR | B | 103 | −76.671 | 37.190 | −13.619 | 1.00 | 37.64 | C |
| ATOM | 1614 | CZ | TYR | B | 103 | −77.946 | 38.354 | −13.847 | 1.00 | 37.92 | C |
| ATOM | 1615 | OH | TYR | B | 103 | −77.995 | 38.973 | −15.074 | 1.00 | 38.27 | O |
| ATOM | 1616 | CE2 | TYR | B | 103 | −77.170 | 36.900 | 12.841 | 1.00 | 37.66 | C |
| ATOM | 1617 | CD2 | TYR | B | 103 | −77.119 | 38.281 | −11.601 | 1.00 | 37.60 | C |
| ATOM | 1618 | C | TYR | B | 103 | −75.945 | 34.885 | −10.800 | 1.00 | 37.35 | C |
| ATOM | 1619 | O | TYR | B | 103 | −75.059 | 35.733 | −10.714 | 1.00 | 37.70 | O |
| ATOM | 1620 | N | GLN | B | 104 | −75.645 | 33.806 | −11.575 | 1.00 | 37.43 | N |
| ATOM | 1621 | CA | GLN | B | 104 | −74.615 | 33.492 | −12.307 | 1.00 | 37.28 | C |
| ATOM | 1622 | CB | GLN | B | 104 | −74.589 | 32.013 | −12.689 | 1.00 | 37.48 | C |
| ATOM | 1623 | CG | GLN | B | 104 | −74.597 | 31.091 | −11.478 | 1.00 | 37.30 | C |
| ATOM | 1624 | CD | GLN | B | 104 | −74.321 | 29.645 | −11.833 | 1.00 | 37.43 | C |
| ATOM | 1625 | OE1 | GLN | B | 104 | −74.500 | 29.226 | −12.977 | 1.00 | 37.32 | O |
| ATOM | 1626 | NE2 | GLN | B | 104 | −73.883 | 28.870 | −10.848 | 1.00 | 36.79 | N |
| ATOM | 1627 | C | GLN | B | 104 | −74.417 | 34.378 | −13.538 | 1.00 | 37.31 | C |
| ATOM | 1628 | O | GLN | B | 104 | −73.288 | 34.765 | −13.846 | 1.00 | 37.51 | O |
| ATOM | 1629 | N | GLY | B | 105 | −75.507 | 34.690 | −14.236 | 1.00 | 37.14 | N |
| ATOM | 1630 | CA | GLY | B | 105 | −75.473 | 35.624 | −15.360 | 1.00 | 37.62 | C |
| ATOM | 1631 | C | GLY | B | 105 | −74.915 | 35.039 | −16.643 | 1.00 | 37.98 | C |
| ATOM | 1632 | O | GLY | B | 105 | −74.696 | 33.830 | −16.749 | 1.00 | 39.23 | O |
| ATOM | 1633 | N | SER | B | 106 | −74.673 | 35.918 | 17.611 | 1.00 | 38.28 | O |
| ATOM | 1634 | CA | SER | B | 106 | −74.211 | 35.522 | −18.965 | 1.00 | 37.96 | C |
| ATOM | 1635 | CB | SER | B | 106 | −74.382 | 36.717 | −19.916 | 1.00 | 38.09 | C |
| ATOM | 1636 | OG | SER | B | 106 | −75.698 | 37.245 | −19.910 | 1.00 | 37.88 | O |
| ATOM | 1637 | C | SER | B | 106 | −72.864 | 34.926 | −19.065 | 1.00 | 37.87 | C |
| ATOM | 1638 | O | SER | B | 106 | −72.594 | 34.136 | −19.971 | 1.00 | 38.03 | O |
| ATOM | 1639 | N | TYR | B | 107 | −71.980 | 35.304 | −18.140 | 1.00 | 37.30 | N |
| ATOM | 1640 | C | TYR | B | 107 | −70.582 | 34.857 | −18.151 | 1.00 | 36.35 | C |
| ATOM | 1641 | CO | TYR | B | 107 | −69.656 | 36.045 | −17.854 | 1.00 | 36.86 | C |
| ATOM | 1642 | CG | TYR | B | 107 | −69.995 | 37.256 | −18.695 | 1.00 | 38.07 | C |
| ATOM | 1643 | CD1 | TYR | B | 107 | −69.548 | 37.362 | −20.015 | 1.00 | 38.23 | C |
| ATOM | 1644 | CE1 | TYR | B | 107 | −69.676 | 38.460 | −20.798 | 1.00 | 38.37 | C |
| ATOM | 1645 | CZ | TYR | B | 107 | −70.667 | 39.469 | −20.266 | 1.00 | 38.55 | C |
| ATOM | 1646 | OH | TYR | B | 107 | −70.993 | 40.557 | −21.039 | 1.00 | 39.13 | O |
| ATOM | 1647 | CE2 | TYR | B | 107 | −71.131 | 39.383 | −18.963 | 1.00 | 38.48 | C |
| ATOM | 1648 | CD2 | TYR | B | 107 | −70.800 | 38.281 | −18.188 | 1.00 | 38.23 | C |
| ATOM | 1649 | C | TYR | B | 107 | −70.311 | 33.691 | −17.192 | 1.00 | 34.76 | C |
| ATOM | 1650 | O | TYR | B | 107 | −69.154 | 33.297 | −17.011 | 1.00 | 35.22 | O |
| ATOM | 1651 | N | GLY | B | 108 | −71.372 | 33.134 | −16.600 | 1.00 | 33.25 | N |
| ATOM | 1652 | CA | GLY | B | 108 | −71.278 | 31.955 | −15.739 | 1.00 | 31.73 | C |
| ATOM | 1653 | C | GLY | B | 108 | −70.431 | 32.192 | −14.510 | 1.00 | 30.37 | C |
| ATOM | 1654 | O | GLY | B | 108 | −69.457 | 31.476 | −14.272 | 1.00 | 30.42 | O |
| ATOM | 1655 | N | PHE | B | 109 | −70.812 | 33.203 | −13.734 | 1.00 | 29.17 | N |
| ATOM | 1656 | CA | PHE | B | 109 | −70.040 | 33.626 | −12.574 | 1.00 | 28.09 | C |
| ATOM | 1657 | CB | PHE | B | 109 | −70.385 | 35.072 | −12.190 | 1.00 | 27.63 | C |
| ATOM | 1658 | CG | PHE | B | 109 | −69.658 | 35.569 | −10.970 | 1.00 | 26.96 | C |
| ATOM | 1659 | CD1 | PHE | B | 109 | −68.276 | 35.418 | −10.852 | 1.00 | 26.87 | C |
| ATOM | 1660 | CE1 | PHE | B | 109 | −67.607 | 35.871 | −9.727 | 1.00 | 26.91 | C |
| ATOM | 1661 | CZ | PHE | B | 109 | −68.310 | 36.487 | −8.704 | 1.00 | 26.81 | C |
| ATOM | 1662 | CE2 | PHE | B | 109 | −69.684 | 36.650 | −8.808 | 1.00 | 26.99 | C |
| ATOM | 1663 | CD2 | PHE | B | 109 | −70.351 | 36.192 | −9.935 | 1.00 | 26.93 | C |
| ATOM | 1664 | C | PHE | B | 109 | −70.259 | 32.688 | −11.388 | 1.00 | 28.00 | C |
| ATOM | 1665 | O | PHE | B | 109 | −71.388 | 35.522 | −10.914 | 1.00 | 28.82 | O |
| ATOM | 1666 | N | ARG | B | 110 | −69.165 | 32.084 | −10.927 | 1.00 | 27.00 | N |
| ATOM | 1667 | CA | ARG | B | 110 | −63.160 | 31.185 | −9.779 | 1.00 | 26.80 | C |
| ATOM | 1668 | CB | ARG | B | 110 | −69.087 | 29.728 | −10.241 | 1.00 | 27.21 | C |
| ATOM | 1669 | CG | ARG | B | 110 | −70.324 | 29.241 | −10.976 | 1.00 | 27.75 | C |
| ATOM | 1670 | CD | ARG | B | 110 | −70.170 | 27.786 | −11.397 | 1.00 | 28.41 | C |
| ATOM | 1671 | NE | ARG | B | 110 | −71.373 | 27.252 | −12.044 | 1.00 | 28.77 | N |
| ATOM | 1672 | CZ | ARG | B | 110 | −71.761 | 27.512 | −13.296 | 1.00 | 29.33 | C |
| ATOM | 1673 | NH1 | ARG | B | 110 | −72.880 | 26.958 | −13.762 | 1.00 | 29.35 | N |
| ATOM | 1674 | NH2 | ARG | B | 110 | −71.055 | 28.323 | −14.091 | 1.00 | 29.80 | N |
| ATOM | 1675 | C | ARG | B | 110 | −67.952 | 31.503 | −8.913 | 1.00 | 26.01 | C |
| ATOM | 1676 | O | ARG | B | 110 | −66.943 | 32.006 | −9.413 | 1.00 | 25.40 | O |
| ATOM | 1677 | N | LEU | B | 111 | −68.067 | 31.210 | −7.619 | 1.00 | 25.41 | N |
| ATOM | 1678 | CA | LEU | B | 111 | −66.952 | 31.338 | −6.682 | 1.00 | 24.90 | C |
| ATOM | 1679 | CB | LEU | B | 111 | −67.431 | 31.909 | −5.342 | 1.00 | 25.19 | C |
| ATOM | 1680 | CG | LEU | B | 111 | −67.976 | 33.343 | −5.369 | 1.00 | 25.34 | C |
| ATOM | 1681 | CD1 | LEU | B | 111 | −68.571 | 33.713 | −4.018 | 1.00 | 25.56 | C |
| ATOM | 1682 | CD2 | LEU | B | 111 | −66.903 | 34.349 | −5.760 | 1.00 | 25.24 | C |
| ATOM | 1683 | C | LEU | B | 111 | −66.272 | 29.989 | −6.466 | 1.00 | 24.23 | C |
| ATOM | 1684 | O | LEU | B | 111 | −66.884 | 28.936 | −6.646 | 1.00 | 24.08 | O |
| ATOM | 1685 | N | GLY | B | 112 | −64.995 | 30.045 | −6.094 | 1.00 | 23.61 | N |
| ATOM | 1686 | CA | GLY | B | 112 | −64.200 | 28.864 | −5.743 | 1.00 | 22.84 | C |
| ATOM | 1687 | C | GLY | B | 112 | −63.255 | 29.216 | −4.610 | 1.00 | 22.46 | C |
| ATOM | 1688 | O | GLY | B | 112 | −62.930 | 30.389 | −4.406 | 1.00 | 21.95 | O |
| ATOM | 1689 | N | PHE | B | 113 | −62.833 | 28.206 | −3.854 | 1.00 | 21.49 | N |
| ATOM | 1690 | CA | PHE | B | 113 | −61.994 | 28.417 | −2.667 | 1.00 | 21.47 | C |
| ATOM | 1691 | CB | PHE | B | 113 | −62.846 | 28.437 | −1.386 | 1.00 | 21.21 | C |
| ATOM | 1692 | CG | PHE | B | 113 | −64.140 | 29.202 | −1.515 | 1.00 | 21.17 | C |
| ATOM | 1693 | CD1 | PHE | B | 113 | −65.263 | 28.605 | −2.072 | 1.00 | 21.05 | C |
| ATOM | 1694 | CE1 | PHE | B | 113 | −66.453 | 29.304 | −2.201 | 1.00 | 21.07 | C |
| ATOM | 1695 | CZ | PHE | B | 113 | −66.535 | 30.616 | −1.757 | 1.00 | 21.04 | C |
| ATOM | 1696 | CE2 | PHE | B | 113 | −65.425 | 31.222 | −1.193 | 1.00 | 21.07 | C |
| ATOM | 1697 | CD2 | PHE | B | 113 | −64.237 | 30.516 | −1.073 | 1.00 | 21.16 | C |
| ATOM | 1698 | C | PHE | B | 113 | −60.948 | 27.311 | −2.571 | 1.00 | 21.11 | C |
| ATOM | 1699 | O | PHE | B | 113 | −61.156 | 26.209 | −3.081 | 1.00 | 20.99 | O |
| ATOM | 1700 | N | LEU | B | 114 | −59.828 | 27.605 | −1.915 | 1.00 | 21.10 | N |
| ATOM | 1701 | CA | LEU | B | 114 | −58.784 | 26.602 | −1.710 | 1.00 | 21.24 | C |
| ATOM | 1702 | CB | LEU | B | 114 | −57.524 | 27.234 | −1.114 | 1.00 | 21.46 | C |
| ATOM | 1703 | CG | LEU | B | 114 | −56.779 | 28.245 | −1.994 | 1.00 | 21.22 | C |
| ATOM | 1704 | CD1 | LEU | B | 114 | −55.626 | 28.871 | −1.226 | 1.00 | 21.30 | C |
| ATOM | 1705 | CD2 | LEU | B | 114 | −56.269 | 27.586 | −3.267 | 1.00 | 21.45 | C |
| ATOM | 1706 | C | LEU | B | 114 | −59.294 | 25.485 | −0.803 | 1.00 | 21.74 | C |
| ATOM | 1707 | O | LEU | B | 114 | −60.107 | 25.726 | 0.100 | 1.00 | 21.33 | O |
| ATOM | 1708 | N | HIS | B | 115 | −58.839 | 24.263 | −1.076 | 1.00 | 22.16 | N |
| ATOM | 1709 | CA | HIS | B | 115 | −59.161 | 23.096 | −0.253 | 1.00 | 22.62 | C |
| ATOM | 1710 | CB | HIS | B | 115 | −59.320 | 21.852 | −1.135 | 1.00 | 23.02 | C |
| ATOM | 1711 | CG | HIS | B | 115 | −60.296 | 22.045 | −2.254 | 1.00 | 23.24 | C |
| ATOM | 1712 | ND1 | HIS | B | 115 | −59.906 | 22.146 | −3.572 | 1.00 | 23.49 | N |
| ATOM | 1713 | CE1 | HIS | B | 115 | −60.970 | 22.348 | −4.329 | 1.00 | 23.46 | C |
| ATOM | 1714 | NE2 | HIS | B | 115 | −62.034 | 22.388 | −3.549 | 1.00 | 23.70 | N |
| ATOM | 1715 | CD2 | HIS | B | 115 | −61.638 | 22.216 | −2.244 | 1.00 | 23.52 | C |
| ATOM | 1716 | C | HIS | B | 115 | −58.054 | 22.961 | 0.784 | 1.00 | 22.73 | C |
| ATOM | 1717 | O | HIS | B | 115 | −57.154 | 22.132 | 0.662 | 1.00 | 22.25 | O |
| ATOM | 1718 | N | SER | B | 116 | −58.146 | 23.808 | 1.807 | 1.00 | 23.19 | N |
| ATOM | 1719 | CA | SER | B | 116 | −57.051 | 24.052 | 2.746 | 1.00 | 23.59 | C |
| ATOM | 1720 | CB | SER | B | 116 | −57.191 | 25.451 | 3.347 | 1.00 | 23.90 | C |
| ATOM | 1721 | OG | SER | B | 116 | −57.357 | 26.422 | 2.332 | 1.00 | 23.53 | O |
| ATOM | 1722 | C | SER | B | 116 | −56.949 | 23.042 | 3.881 | 1.00 | 24.07 | C |
| ATOM | 1723 | O | SER | B | 116 | −55.873 | 22.865 | 4.444 | 1.00 | 24.03 | O |
| ATOM | 1724 | N | GLY | B | 117 | −58.064 | 22.396 | 4.220 | 1.00 | 24.74 | N |
| ATOM | 1725 | CA | GLY | B | 117 | −58.123 | 21.486 | 5.360 | 1.00 | 24.96 | C |
| ATOM | 1726 | C | GLY | B | 117 | −58.411 | 22.222 | 6.655 | 1.00 | 25.59 | C |
| ATOM | 1727 | O | GLY | B | 117 | −58.737 | 23.414 | 6.642 | 1.00 | 25.21 | O |
| ATOM | 1728 | N | THR | B | 118 | −58.274 | 21.506 | 7.771 | 1.00 | 25.98 | N |
| ATOM | 1729 | CA | THR | B | 118 | −58.613 | 22.022 | 9.098 | 1.00 | 26.57 | C |
| ATOM | 1730 | CB | THR | B | 118 | −59.917 | 21.371 | 9.605 | 1.00 | 26.20 | C |
| ATOM | 1731 | OG1 | THR | B | 118 | −59.763 | 19.946 | 9.646 | 1.00 | 26.63 | O |
| ATOM | 1732 | CG2 | THR | B | 118 | −61.083 | 21.745 | 8.702 | 1.00 | 25.80 | C |
| ATOM | 1733 | C | THR | B | 118 | −57.505 | 21.809 | 10.141 | 1.00 | 27.37 | C |
| ATOM | 1734 | O | THR | B | 118 | −57.793 | 21.708 | 11.335 | 1.00 | 26.63 | O |
| ATOM | 1735 | N | ALA | B | 119 | −56.246 | 21.767 | 9.700 | 1.00 | 29.06 | N |
| ATOM | 1736 | CA | ALA | B | 119 | −55.109 | 21.608 | 10.620 | 1.00 | 30.58 | C |
| ATOM | 1737 | CB | ALA | B | 119 | −53.842 | 21.273 | 9.851 | 1.00 | 30.37 | C |
| ATOM | 1738 | C | ALA | B | 119 | −54.898 | 22.872 | 11.454 | 1.00 | 32.18 | C |
| ATOM | 1739 | O | ALA | B | 119 | −55.346 | 23.951 | 11.067 | 1.00 | 32.62 | O |
| ATOM | 1740 | N | LYS | B | 120 | −54.205 | 22.734 | 12.587 | 1.00 | 33.74 | N |
| ATOM | 1741 | CA | LYS | B | 120 | −53.914 | 23.870 | 13.488 | 1.00 | 34.69 | C |
| ATOM | 1742 | CB | LYS | B | 120 | −52.916 | 23.465 | 14.580 | 1.00 | 35.55 | C |
| ATOM | 1743 | CG | LYS | B | 120 | −53.453 | 22.535 | 15.654 | 1.00 | 36.30 | C |
| ATOM | 1744 | CD | LYS | B | 120 | −52.316 | 22.028 | 16.531 | 1.00 | 36.63 | C |
| ATOM | 1745 | CE | LYS | B | 120 | −52.804 | 21.054 | 17.588 | 1.00 | 36.89 | C |
| ATOM | 1746 | NZ | LYS | B | 120 | −53.769 | 21.684 | 18.531 | 1.00 | 37.13 | N |
| ATOM | 1747 | C | LYS | B | 120 | −53.342 | 25.090 | 12.759 | 1.00 | 34.86 | C |
| ATOM | 1748 | O | LYS | B | 120 | −53.729 | 26.229 | 13.039 | 1.00 | 35.39 | O |
| ATOM | 1749 | N | SER | B | 121 | −52.428 | 24.836 | 11.822 | 1.00 | 34.61 | N |
| ATOM | 1750 | CA | SER | B | 121 | −51.695 | 25.896 | 11.130 | 1.00 | 33.86 | C |
| ATOM | 1751 | CB | SER | B | 121 | −50.367 | 25.350 | 10.591 | 1.00 | 33.90 | C |
| ATOM | 1752 | OG | SER | B | 121 | −50.566 | 24.498 | 9.475 | 1.00 | 33.91 | O |
| ATOM | 1753 | C | SER | B | 121 | −52.459 | 26.588 | 9.993 | 1.00 | 33.21 | C |
| ATOM | 1754 | O | SER | B | 121 | −51.921 | 27.526 | 9.398 | 1.00 | 33.66 | O |
| ATOM | 1755 | N | VAL | B | 122 | −53.679 | 26.139 | 9.676 | 1.00 | 32.11 | N |
| ATOM | 1756 | CA | VAL | B | 122 | −54.491 | 26.809 | 8.653 | 1.00 | 31.37 | C |
| ATOM | 1757 | CB | VAL | B | 122 | −55.742 | 25.976 | 8.236 | 1.00 | 31.87 | C |
| ATOM | 1758 | CG1 | VAL | B | 122 | −56.855 | 26.031 | 9.280 | 1.00 | 31.76 | C |
| ATOM | 1759 | CG2 | VAL | B | 122 | −56.270 | 26.436 | 6.883 | 1.00 | 31.95 | C |
| ATOM | 1760 | C | VAL | B | 122 | −54.876 | 28.213 | 9.139 | 1.00 | 30.21 | C |
| ATOM | 1761 | O | VAL | B | 122 | −55.268 | 28.388 | 10.296 | 1.00 | 30.74 | O |
| ATOM | 1762 | N | THR | B | 123 | −54.692 | 29.207 | 8.271 | 1.00 | 28.68 | N |
| ATOM | 1763 | CA | THR | B | 123 | −55.069 | 30.595 | 8.555 | 1.00 | 27.78 | C |
| ATOM | 1764 | CB | THR | B | 123 | −53.939 | 31.565 | 8.168 | 1.00 | 27.30 | C |
| ATOM | 1765 | OG1 | THR | B | 123 | −53.650 | 31.437 | 6.769 | 1.00 | 27.32 | O |
| ATOM | 1765 | CG2 | THR | B | 123 | −52.680 | 31.268 | 8.976 | 1.00 | 27.25 | C |
| ATOM | 1767 | C | THR | B | 123 | −56.359 | 31.015 | 7.842 | 1.00 | 27.38 | C |
| ATOM | 1768 | O | THR | B | 123 | −56.968 | 32.017 | 8.212 | 1.00 | 26.62 | O |
| ATOM | 1769 | N | CYS | B | 124 | −56.760 | 30.261 | 6.820 | 1.00 | 27.47 | N |
| ATOM | 1770 | CA | CYS | B | 124 | −58.006 | 30.503 | 6.098 | 1.00 | 27.61 | C |
| ATOM | 1771 | CB | CYS | B | 124 | −57.775 | 31.523 | 4.985 | 1.00 | 28.88 | C |
| ATOM | 1772 | SG | CYS | B | 124 | −59.210 | 31.821 | 3.929 | 1.00 | 32.04 | S |
| ATOM | 1773 | C | CYS | B | 124 | −58.509 | 29.185 | 5.511 | 1.00 | 26.28 | C |
| ATOM | 1774 | O | CYS | B | 124 | −57.750 | 28.484 | 4.835 | 1.00 | 26.84 | O |
| ATOM | 1775 | N | THR | B | 125 | −59.774 | 28.847 | 5.769 | 1.00 | 24.72 | N |
| ATOM | 1776 | CA | THR | B | 125 | −60.350 | 27.580 | 5.296 | 1.00 | 23.54 | C |
| ATOM | 1777 | CB | THR | B | 125 | −60.046 | 24.411 | 6.265 | 1.00 | 23.49 | C |
| ATOM | 1778 | OG1 | THR | B | 125 | −60.468 | 25.174 | 5.680 | 1.00 | 23.60 | O |
| ATOM | 1779 | CG2 | THR | B | 125 | −60.728 | 26.592 | 7.626 | 1.00 | 23.56 | C |
| ATOM | 1780 | C | THR | B | 125 | −61.853 | 27.692 | 5.047 | 1.00 | 22.60 | C |
| ATOM | 1781 | O | THR | B | 125 | −62.571 | 23.358 | 5.795 | 1.00 | 22.47 | O |
| ATOM | 1782 | N | TYR | B | 126 | −62.309 | 27.021 | 3.993 | 1.00 | 21.27 | N |
| ATOM | 1783 | CA | TYR | B | 126 | −63.689 | 27.102 | 3.350 | 1.00 | 20.74 | C |
| ATOM | 1784 | CB | TYR | B | 126 | −63.699 | 27.459 | 2.043 | 1.00 | 20.11 | C |
| ATOM | 1785 | CG | TYR | B | 126 | −65.063 | 27.389 | 1.403 | 1.00 | 20.04 | C |
| ATOM | 1786 | CD1 | TYR | B | 126 | −65.994 | 28.413 | 1.603 | 1.00 | 19.90 | C |
| ATOM | 1787 | CE1 | TYR | B | 126 | −67.250 | 28.356 | 1.021 | 1.00 | 19.95 | C |
| ATOM | 1788 | CZ | TYR | B | 126 | −67.585 | 27.276 | 0.226 | 1.00 | 19.80 | C |
| ATOM | 1789 | OH | TYR | B | 126 | −68.818 | 27.205 | −0.366 | 1.00 | 19.83 | O |
| ATOM | 1790 | CE2 | TYR | B | 126 | −66.677 | 26.259 | 0.010 | 1.00 | 19.98 | C |
| ATOM | 1791 | CD2 | TYR | B | 126 | −64.425 | 26.323 | 0.596 | 1.00 | 20.00 | C |
| ATOM | 1792 | C | TYR | B | 126 | −64.413 | 25.776 | 3.767 | 1.00 | 20.50 | C |
| ATOM | 1793 | O | TYR | B | 126 | −63.847 | 24.710 | 3.533 | 1.00 | 19.68 | O |
| ATOM | 1794 | N | SER | B | 127 | −65.663 | 25.853 | 4.223 | 1.00 | 20.45 | N |
| ATOM | 1795 | CA | SER | B | 127 | −66.531 | 24.685 | 4.348 | 1.00 | 20.29 | C |
| ATOM | 1796 | CB | SER | B | 127 | −67.284 | 24.714 | 5.677 | 1.00 | 20.33 | C |
| ATOM | 1797 | OG | SER | B | 127 | −68.131 | 25.583 | 5.796 | 1.00 | 20.27 | O |
| ATOM | 1798 | C | SER | B | 127 | −67.531 | 24.656 | 3.189 | 1.00 | 20.26 | C |
| ATOM | 1799 | O | SER | B | 127 | −68.358 | 25.564 | 3.075 | 1.00 | 20.10 | O |
| ATOM | 1800 | N | PRO | B | 128 | −67.441 | 23.632 | 2.311 | 1.00 | 20.25 | N |
| ATOM | 1801 | CA | PRO | B | 128 | −68.493 | 23.369 | 1.322 | 1.00 | 20.25 | C |
| ATOM | 1802 | CB | PRO | B | 128 | −67.944 | 22.171 | 0.537 | 1.00 | 20.29 | C |
| ATOM | 1803 | CO | PRO | B | 128 | −66.470 | 22.292 | 0.657 | 1.00 | 20.28 | C |
| ATOM | 1804 | CD | PRO | B | 128 | −66.228 | 22.840 | 2.030 | 1.00 | 20.33 | C |
| ATOM | 1805 | C | PRO | B | 128 | −69.843 | 23.020 | 1.949 | 1.00 | 20.06 | C |
| ATOM | 1806 | O | PRO | B | 128 | −70.877 | 23.486 | 1.469 | 1.00 | 19.98 | O |
| ATOM | 1807 | N | ALA | B | 129 | −69.818 | 22.209 | 3.007 | 1.00 | 20.27 | N |
| ATOM | 1808 | CA | ALA | B | 129 | −71.027 | 21.825 | 3.746 | 1.00 | 20.59 | C |
| ATOM | 1809 | CB | ALA | B | 129 | −70.675 | 20.837 | 4.852 | 1.00 | 20.50 | C |
| ATOM | 1810 | C | ALA | B | 129 | −71.789 | 23.020 | 4.328 | 1.00 | 21.01 | C |
| ATOM | 1811 | O | ALA | B | 129 | −73.019 | 23.029 | 4.316 | 1.00 | 21.00 | O |
| ATOM | 1812 | N | LEU | B | 130 | −71.060 | 24.023 | 4.820 | 1.00 | 21.78 | N |
| ATOM | 1813 | CA | LEU | B | 130 | −71−666 | 25.217 | 5.430 | 1.00 | 22.18 | C |
| ATOM | 1814 | CB | LEU | B | 130 | −70.924 | 25.562 | 6.725 | 1.00 | 22.31 | C |
| ATOM | 1815 | CG | LEU | B | 130 | −70.693 | 24.443 | 7.750 | 1.00 | 22.24 | C |
| ATOM | 1816 | C01 | LEU | B | 130 | −69.703 | 24.895 | 8.813 | 1.00 | 22.35 | C |
| ATOM | 1817 | Cr.2 | LEU | B | 130 | −72.001 | 23.993 | 8.384 | 1.00 | 22.18 | C |
| ATOM | 1818 | C | LEU | B | 130 | −71.672 | 26.456 | 4.517 | 1.00 | 22.84 | C |
| ATOM | 1819 | O | LEU | B | 130 | −72.217 | 27.496 | 4.904 | 1.00 | 23.35 | O |
| ATOM | 1820 | N | ASN | B | 131 | −71.088 | 26.346 | 3.318 | 1.00 | 23.09 | N |
| ATOM | 1821 | CA | ASN | B | 131 | −70.750 | 27.510 | 2.470 | 1.00 | 22.74 | C |
| ATOM | 1822 | CB | ASN | B | 131 | −71−939 | 27.934 | 1.587 | 1.00 | 22.89 | C |
| ATOM | 1823 | CG | ASN | B | 131 | −71.547 | 28.933 | 0.498 | 1.00 | 23.23 | C |
| ATOM | 1824 | OD1 | ASN | B | 131 | −70.374 | 29.081 | 0.155 | 1.00 | 23.14 | O |
| ATOM | 1825 | ND2 | ASN | B | 131 | −72.539 | 29.625 | −0.048 | 1.00 | 23.38 | N |
| ATOM | 1826 | C | ASN | B | 131 | −70.235 | 28.662 | 3.337 | 1.00 | 22.58 | C |
| ATOM | 1827 | O | ASN | B | 131 | −70.809 | 29.755 | 3.375 | 1.00 | 22.46 | O |
| ATOM | 1828 | N | LYS | B | 132 | −69.151 | 28.379 | 4.052 | 1.00 | 22.80 | N |
| ATOM | 1829 | CA | LYS | B | 132 | −68.679 | 29.247 | 5.119 | 1.00 | 22.74 | C |
| ATOM | 1830 | CB | LYS | B | 132 | −69.175 | 28.736 | 6.471 | 1.00 | 22.45 | C |
| ATOM | 1831 | CG | LYS | B | 132 | −68.891 | 29.673 | 7.634 | 1.00 | 22.59 | C |
| ATOM | 1832 | CD | LYS | B | 132 | −69.639 | 29.235 | 8.879 | 1.00 | 22.77 | C |
| ATOM | 1833 | CE | LYS | B | 132 | −69.417 | 30.190 | 10.039 | 1.00 | 22.96 | C |
| ATOM | 1834 | NZ | LYS | B | 132 | −70.459 | 30.014 | 11.091 | 1.00 | 22.96 | N |
| ATOM | 1835 | C | LYS | B | 132 | −67.165 | 29.316 | 5.129 | 1.00 | 23.01 | C |
| ATOM | 1836 | O | LYS | B | 132 | −66.493 | 28.288 | 5.192 | 1.00 | 22.59 | O |
| ATOM | 1837 | N | MET | B | 133 | −66.649 | 30.537 | 5.079 | 1.00 | 23.37 | N |
| ATOM | 1838 | CA | MET | B | 133 | −65.227 | 30.794 | 5.193 | 1.00 | 24.69 | C |
| ATOM | 1839 | CB | MET | B | 133 | −64.876 | 32.052 | 4.404 | 1.00 | 25.69 | C |
| ATOM | 1840 | CG | MET | B | 133 | −63.395 | 32.351 | 4.341 | 1.00 | 26.72 | C |
| ATOM | 1841 | SD | MET | B | 133 | −65.541 | 31.280 | 3.180 | 1.00 | 28.42 | S |
| ATOM | 1842 | CE | MET | B | 133 | −62.927 | 32.123 | 1.652 | 1.00 | 28.32 | C |
| ATOM | 1843 | C | MET | B | 133 | −64.867 | 30.984 | 6.665 | 1.00 | 24.59 | C |
| ATOM | 1844 | O | MET | B | 133 | −65.599 | 31.640 | 7.406 | 1.00 | 24.31 | O |
| ATOM | 1845 | N | PHE | B | 134 | −63.733 | 30.419 | 7.069 | 1.00 | 24.91 | N |
| ATOM | 1846 | CA | PHE | B | 134 | −63.191 | 30.586 | 8.413 | 1.00 | 25.88 | C |
| ATOM | 1847 | CB | PHE | B | 134 | −63.045 | 29.225 | 9.096 | 1.00 | 25.87 | C |
| ATOM | 1848 | CG | PHE | B | 134 | −64.358 | 25.587 | 9.457 | 1.00 | 25.51 | C |
| ATOM | 1849 | CD1 | PHE | B | 134 | −65.157 | 28.003 | 8.476 | 1.00 | 25.48 | C |
| ATOM | 1850 | CE1 | PHE | B | 134 | −66.372 | 27.417 | 8.804 | 1.00 | 25.35 | C |
| ATOM | 1851 | CZ | PHE | B | 134 | −66.803 | 27.409 | 10.122 | 1.00 | 25.26 | C |
| ATOM | 1852 | CE2 | PHE | B | 134 | −66.016 | 27.984 | 11.110 | 1.00 | 25.40 | C |
| ATOM | 1853 | CD2 | PHE | B | 134 | −64.802 | 28.567 | 10.778 | 1.00 | 25.51 | C |
| ATOM | 1854 | C | PHE | B | 134 | −61.838 | 31.283 | 8.295 | 1.00 | 26.99 | C |
| ATOM | 1855 | O | PHE | B | 134 | −60.898 | 30.701 | 7.767 | 1.00 | 26.09 | O |
| ATOM | 1856 | N | CYS | B | 135 | −61.760 | 32.525 | 8.782 | 1.00 | 29.19 | N |
| ATOM | 1857 | CA | CYS | B | 135 | −60.578 | 33.389 | 8.641 | 1.00 | 30.69 | C |
| ATOM | 1858 | CB | CYS | B | 135 | −60.959 | 34.681 | 7.919 | 1.00 | 32.36 | C |
| ATOM | 1859 | SG | CYS | B | 135 | −61.559 | 34.454 | 6.239 | 1.00 | 36.51 | S |
| ATOM | 1860 | C | CYS | B | 135 | −59.995 | 33.790 | 9.988 | 1.00 | 30.21 | C |
| ATOM | 1861 | O | CYS | B | 135 | −60.720 | 33.915 | 10.972 | 1.00 | 30.49 | O |
| ATOM | 1862 | N | GLN | B | 136 | −58.684 | 34.007 | 10.016 | 1.00 | 29.43 | N |
| ATOM | 1863 | CA | GLN | B | 136 | −58.032 | 34.688 | 11.136 | 1.00 | 29.04 | C |
| ATOM | 1864 | CB | GLN | B | 136 | −56.567 | 34.276 | 11.241 | 1.00 | 28.92 | C |
| ATOM | 1865 | CO | GLN | B | 136 | −56.359 | 32.820 | 11.618 | 1.00 | 29.27 | C |
| ATOM | 1866 | CD | GLN | B | 136 | −54.912 | 32.500 | 11.936 | 1.00 | 29.22 | C |
| ATOM | 1867 | OE1 | GLN | B | 136 | −53.993 | 33.063 | 11.340 | 1.00 | 29.54 | O |
| ATOM | 1868 | OE2 | GLN | B | 136 | −54.702 | 31.594 | 12.880 | 1.00 | 28.79 | N |
| ATOM | 1869 | C | GLN | B | 136 | −58.157 | 36.195 | 10.927 | 1.00 | 28.18 | C |
| ATOM | 1870 | O | GLN | B | 136 | −58.413 | 36.648 | 9.808 | 1.00 | 28.42 | O |
| ATOM | 1871 | N | LEU | B | 137 | −57.981 | 36.963 | 12.002 | 1.00 | 27.96 | N |
| ATOM | 1872 | CA | LEU | B | 137 | −58.142 | 38.422 | 11.952 | 1.00 | 27.37 | C |
| ATOM | 1873 | CB | LEU | B | 137 | −58.053 | 39.034 | 13.356 | 1.00 | 27.09 | C |
| ATOM | 1874 | CG | LEU | B | 137 | −58.215 | 40.556 | 13.471 | 1.00 | 27.19 | C |
| ATOM | 1875 | CD1 | LEU | B | 137 | −59.582 | 40.997 | 12.98/2 | 1.00 | 27.06 | C |
| ATOM | 1876 | CD2 | LEU | B | 137 | −57.991 | 41.016 | 14.904 | 1.00 | 27.42 | C |
| ATOM | 1877 | C | LEU | B | 137 | −57.104 | 39.079 | 11.035 | 1.00 | 27.43 | C |
| ATOM | 1878 | O | LEU | B | 137 | −55.900 | 38.899 | 11.222 | 1.00 | 27.02 | O |
| ATOM | 1879 | N | ALA | B | 138 | −57.594 | 39.817 | 10.038 | 1.00 | 27.68 | N |
| ATOM | 1880 | CA | ALA | B | 138 | −56.764 | 40.599 | 9.109 | 1.00 | 28.32 | C |
| ATOM | 1881 | CB | ALA | B | 138 | −55.956 | 41.648 | 9.875 | 1.00 | 28.40 | C |
| ATOM | 1882 | C | ALA | B | 138 | −55.841 | 39.794 | 8.169 | 1.00 | 28.77 | C |
| ATOM | 1883 | O | ALA | B | 138 | −55.052 | 40.395 | 7.437 | 1.00 | 29.15 | O |
| ATOM | 1884 | N | LYS | B | 139 | −55.945 | 38.463 | 8.160 | 1.00 | 29.51 | N |
| ATOM | 1885 | CA | LYS | B | 139 | −55.041 | 37.630 | 7.360 | 1.00 | 29.97 | C |
| ATOM | 1886 | CB | LYS | B | 139 | −54.882 | 36.237 | 7.975 | 1.00 | 30.20 | C |
| ATOM | 1887 | CG | LYS | B | 139 | −54.388 | 36.218 | 9.416 | 1.00 | 30.43 | C |
| ATOM | 1888 | CD | LYS | B | 139 | −53.036 | 36.891 | 9.598 | 1.00 | 30.95 | C |
| ATOM | 1889 | CE | LYS | B | 139 | −52.515 | 36.719 | 11.017 | 1.00 | 30.99 | C |
| ATOM | 1890 | NZ | LYS | B | 139 | −51.028 | 36.747 | 11.057 | 1.00 | 30.94 | N |
| ATOM | 1891 | C | LYS | B | 139 | −55.553 | 37.504 | 5.935 | 1.00 | 30.48 | C |
| ATOM | 1892 | O | LYS | B | 139 | −56.698 | 37.870 | 5.637 | 1.00 | 30.94 | O |
| ATOM | 1893 | N | THR | B | 140 | −54.696 | 36.980 | 5.059 | 1.00 | 30.59 | N |
| ATOM | 1894 | CA | THR | B | 140 | −55.046 | 36.775 | 3.657 | 1.00 | 30.52 | C |
| ATOM | 1895 | CB | THR | B | 140 | −53.848 | 36.278 | 2.825 | 1.00 | 30.77 | C |
| ATOM | 1896 | OG1 | THR | B | 140 | −52.175 | 37.122 | 3.057 | 1.00 | 31.01 | O |
| ATOM | 1897 | CG1 | THR | B | 140 | −54.184 | 36.280 | 1.338 | 1.00 | 30.83 | C |
| ATOM | 1898 | C | THR | B | 140 | −56.163 | 35.744 | 3.545 | 1.00 | 30.32 | C |
| ATOM | 1899 | O | THR | B | 140 | −56.082 | 34.662 | 4.136 | 1.00 | 29.82 | O |
| ATOM | 1900 | N | CYS | B | 141 | −57.199 | 36.099 | 2.790 | 1.00 | 29.85 | N |
| ATOM | 1901 | CA | CYS | B | 141 | −58.350 | 35.239 | 2.586 | 1.00 | 30.33 | C |
| ATOM | 1902 | CB | CYS | B | 141 | −59.554 | 35.791 | 3.348 | 1.00 | 31.71 | C |
| ATOM | 1903 | SG | CYS | B | 141 | −60.950 | 34.656 | 3.372 | 1.00 | 34.64 | S |
| ATOM | 1904 | C | CYS | B | 141 | −58.629 | 35.112 | 1.050 | 1.00 | 28.52 | C |
| ATOM | 1905 | O | CYS | B | 141 | −59.384 | 35.912 | 0.521 | 1.00 | 28.14 | O |
| ATOM | 1906 | N | PRO | B | 142 | −57.997 | 34.116 | 0.415 | 1.00 | 27.14 | N |
| ATOM | 1907 | CA | PRO | B | 142 | −58.162 | 33.967 | −1.033 | 1.00 | 26.42 | C |
| ATOM | 1908 | CB | PRO | B | 142 | −57.148 | 32.872 | −1.407 | 1.00 | 26.66 | C |
| ATOM | 1909 | CG | PRO | B | 142 | −56.265 | 32.705 | −0.221 | 1.00 | 26.90 | C |
| ATOM | 1910 | CD | PRO | B | 142 | −57.102 | 33.078 | 0.960 | 1.00 | 27.10 | C |
| ATOM | 1911 | C | PRO | B | 142 | −59.574 | 33.544 | −1.434 | 1.00 | 25.37 | C |
| ATOM | 1912 | O | PRO | B | 142 | −60.124 | 32.602 | −0.858 | 1.00 | 25.05 | O |
| ATOM | 1913 | N | VAL | B | 143 | −60.140 | 34.257 | −2.404 | 1.00 | 24.37 | N |
| ATOM | 1914 | CA | VAL | B | 143 | −61.460 | 33.969 | −2.956 | 1.00 | 24.00 | C |
| ATOM | 1915 | CB | VAL | B | 143 | −62.483 | 35.050 | −2.548 | 1.00 | 23.98 | C |
| ATOM | 1916 | CG1 | VAL | B | 143 | −63.827 | 34.822 | −3.236 | 1.00 | 23.94 | C |
| ATOM | 1917 | CG2 | VAL | B | 143 | −62.642 | 35.083 | −1.030 | 1.00 | 24.12 | C |
| ATOM | 1918 | C | VAL | B | 143 | −61−311 | 33.949 | −4.475 | 1.00 | 24.00 | C |
| ATOM | 1919 | O | VAL | B | 143 | −60.916 | 34.953 | −5.065 | 1.00 | 23.78 | O |
| ATOM | 1920 | N | GLN | B | 144 | −61.620 | 32.810 | −5.095 | 1.00 | 23.83 | N |
| ATOM | 1921 | CA | GLN | B | 144 | −61.405 | 32.613 | −6.529 | 1.00 | 24.01 | C |
| ATOM | 1922 | CB | GLN | B | 144 | −60.951 | 31.177 | −6.821 | 1.00 | 23.82 | C |
| ATOM | 1923 | CG | GLN | B | 144 | −59.793 | 30.674 | −5.963 | 1.00 | 23.71 | C |
| ATOM | 1924 | CD | GLN | B | 144 | −59.521 | 29.191 | −6.139 | 1.00 | 23.57 | C |
| ATOM | 1925 | OE1 | GLN | B | 144 | −60.395 | 28.423 | −6.546 | 1.00 | 23.56 | O |
| ATOM | 1926 | NE2 | GLN | B | 144 | −58.302 | 28.778 | −5.824 | 1.00 | 23.49 | N |
| ATOM | 1927 | C | GLN | B | 144 | −62.683 | 32.909 | −7.312 | 1.00 | 24.29 | C |
| ATOM | 1928 | O | GLN | B | 144 | −63.775 | 32.523 | −6.891 | 1.00 | 24.15 | O |
| ATOM | 1929 | N | LEU | B | 145 | −62.534 | 33.607 | −8.436 | 1.00 | 24.88 | N |
| ATOM | 1930 | CA | LEU | B | 145 | −63.629 | 33.884 | −9.362 | 1.00 | 25.69 | C |
| ATOM | 1931 | CB | LEU | B | 145 | −63.548 | 35.322 | −9.887 | 1.00 | 25.60 | C |
| ATOM | 1932 | CG | LEU | B | 145 | −63.244 | 36.429 | −8.876 | 1.00 | 25.42 | C |
| ATOM | 1933 | CD1 | LEU | B | 145 | −63.275 | 37.779 | −9.571 | 1.00 | 25.41 | C |
| ATOM | 1934 | CD2 | LEU | B | 145 | −64.209 | 36.406 | −7.700 | 1.00 | 25.28 | C |
| ATOM | 1935 | C | LEU | B | 145 | −63.521 | 32.911 | −10.530 | 1.00 | 27.03 | C |
| ATOM | 1936 | O | LEU | B | 145 | −62.454 | 32.787 | −11.139 | 1.00 | 27.63 | O |
| ATOM | 1937 | N | TRP | B | 146 | −64.620 | 32.219 | −10.824 | 1.00 | 28.21 | N |
| ATOM | 1938 | CA | TRP | B | 146 | −64.689 | 31.273 | −11.929 | 1.00 | 29.08 | C |
| ATOM | 1939 | CB | TRP | B | 146 | −65.009 | 29.871 | −11.415 | 1.00 | 28.72 | C |
| ATOM | 1940 | CG | TRP | B | 146 | −63.867 | 29.222 | −10.713 | 1.00 | 28.97 | C |
| ATOM | 1941 | CD1 | TRP | B | 146 | −63.418 | 29.495 | −9.456 | 1.00 | 29.10 | C |
| ATOM | 1942 | NE1 | TRP | B | 146 | −62.348 | 28.692 | −9.148 | 1.00 | 29.21 | N |
| ATOM | 1943 | CE2 | TRP | B | 146 | −62.087 | 27.872 | −10.214 | 1.00 | 29.34 | C |
| ATOM | 1944 | CD2 | TRP | B | 146 | −63.026 | 28.181 | −11.223 | 1.00 | 29.13 | C |
| ATOM | 1945 | CE3 | TRP | B | 146 | −62.975 | 27.476 | −12.435 | 1.00 | 29.27 | C |
| ATOM | 1946 | CZ3 | TRP | B | 146 | −61.994 | 26.495 | −12.599 | 1.00 | 29.66 | C |
| ATOM | 1947 | CH2 | TRP | B | 146 | −61.069 | 26.214 | −11.574 | 1.00 | 29.68 | C |
| ATOM | 1948 | CZ2 | TRP | B | 146 | −61.099 | 26.892 | −10.379 | 1.00 | 29.48 | C |
| ATOM | 1949 | C | TRP | B | 146 | −65.768 | 31.732 | −12.898 | 1.00 | 30.34 | C |
| ATOM | 1950 | O | TRP | B | 146 | −66.956 | 31.699 | −12.566 | 1.00 | 30.59 | O |
| ATOM | 1951 | N | VAL | B | 147 | −65.341 | 32.172 | −14.083 | 1.00 | 31.26 | N |
| ATOM | 1952 | CA | VAL | B | 147 | −66.258 | 32.547 | −15.159 | 1.00 | 32.06 | C |
| ATOM | 1953 | CB | VAL | B | 147 | −66.052 | 34.000 | −15.658 | 1.00 | 32.41 | C |
| ATOM | 1954 | CG1 | VAL | B | 147 | −66.481 | 34.997 | −14.590 | 1.00 | 32.52 | C |
| ATOM | 1955 | CG2 | VAL | B | 147 | −64.614 | 34.274 | −16.069 | 1.00 | 32.64 | C |
| ATOM | 1956 | C | VAL | B | 147 | −66.118 | 31.559 | −16.319 | 1.00 | 32.81 | C |
| ATOM | 1957 | O | VAL | B | 147 | −65.002 | 31.176 | −16.687 | 1.00 | 32.47 | O |
| ATOM | 1958 | N | AASP | B | 148 | −67.259 | 31.142 | −16.864 | 1.00 | 33.08 | N |
| ATOM | 1959 | N | BASP | B | 148 | −67.251 | 31.143 | −16.883 | 1.00 | 33.41 | N |
| ATOM | 1960 | CA | AASP | B | 148 | −67.311 | 30.287 | −18.045 | 1.00 | 33.65 | C |
| ATOM | 1961 | CA | BASP | B | 148 | −67.259 | 30.260 | −18.047 | 1.00 | 34.19 | C |
| ATOM | 1962 | CB | AASP | B | 148 | −68.697 | 29.659 | −18.160 | 1.00 | 33.13 | C |
| ATOM | 1963 | CB | BASP | B | 148 | −68.505 | 29.360 | −18.020 | 1.00 | 34.03 | C |
| ATOM | 1964 | CA | AASP | B | 148 | −69.016 | 28.745 | −17.001 | 1.00 | 32.57 | C |
| ATOM | 1965 | CG | BASP | B | 148 | −69.765 | 30.075 | −18.462 | 1.00 | 33.99 | C |
| ATOM | 1966 | OD1 | AASP | B | 148 | −69.076 | 28.137 | −16.447 | 1.00 | 32.08 | O |
| ATOM | 1967 | OD1 | BASP | B | 148 | −69.664 | 31.194 | −19.005 | 1.00 | 33.96 | O |
| ATOM | 1968 | OD2 | AASP | B | 148 | −70.205 | 28.635 | −16.647 | 1.00 | 32.38 | O |
| ATOM | 1969 | OD2 | BASP | B | 148 | −70.864 | 29.507 | −18.275 | 0.50 | 33.43 | O |
| ATOM | 1970 | C | AASP | B | 148 | −66.982 | 31.058 | −19.326 | 0.50 | 34.49 | C |
| ATOM | 1971 | C | BASP | B | 148 | −67.133 | 31.037 | −19.376 | 0.50 | 34.84 | C |
| ATOM | 1972 | O | AASP | B | 148 | −66.505 | 30.469 | −20.299 | 0.50 | 35.09 | O |
| ATOM | 1973 | O | BASP | B | 148 | −66.968 | 30.426 | −20.435 | 0.50 | 35.56 | O |
| ATOM | 1974 | N | SER | B | 149 | −67.232 | 32.369 | −19.314 | 1.00 | 35.22 | N |
| ATOM | 1975 | CA | SER | B | 149 | −66.913 | 33.259 | −20.441 | 1.00 | 35.79 | C |
| ATOM | 1976 | CB | SER | B | 149 | −68.193 | 33.725 | −21.143 | 1.00 | 35.45 | C |
| ATOM | 1977 | OG | SER | B | 149 | −69.072 | 32.646 | −21.415 | 1.00 | 35.93 | O |
| ATOM | 1978 | C | SER | B | 149 | −66.160 | 34.483 | −19.905 | 1.00 | 36.63 | C |
| ATOM | 1979 | O | SER | B | 149 | −66.368 | 34.888 | −18.760 | 1.00 | 36.45 | O |
| ATOM | 1980 | N | TOR | B | 150 | −65.293 | 35.069 | −20.730 | 1.00 | 37.62 | N |
| ATOM | 1981 | CA | TOR | B | 150 | −64.583 | 36.298 | −20.360 | 1.00 | 37.73 | C |
| ATOM | 1982 | CB | TOR | B | 150 | −63.402 | 36.591 | −21.316 | 1.00 | 38.11 | C |
| ATOM | 1983 | OG1 | TOR | B | 150 | −62.497 | 35.480 | −21.327 | 1.00 | 37.98 | O |
| ATOM | 1984 | CG2 | TOR | B | 150 | −62.641 | 37.852 | −20.889 | 1.00 | 38.08 | C |
| ATOM | 1985 | C | TOR | B | 150 | −65.555 | 37.486 | −20.401 | 1.00 | 37.70 | C |
| ATOM | 1986 | O | TOR | B | 150 | −66.136 | 37.757 | −21.456 | 1.00 | 37.58 | O |
| ATOM | 1987 | N | PRO | B | 151 | −65.750 | 38.186 | −19.259 | 1.00 | 37.25 | N |
| ATOM | 1988 | CA | PRO | B | 151 | −66.531 | 39.421 | −19.306 | 1.00 | 37.28 | C |
| ATOM | 1989 | CB | PRO | B | 151 | −66.890 | 39.659 | −17.839 | 1.00 | 37.38 | C |
| ATOM | 1990 | CG | PRO | B | 151 | −65.741 | 39.090 | −17.088 | 1.00 | 37.26 | C |
| ATOM | 1991 | CD | PRO | B | 151 | −65.221 | 37.934 | −17.903 | 1.00 | 37.06 | C |
| ATOM | 1992 | C | PRO | B | 151 | −65.676 | 40.564 | −19.865 | 1.00 | 37.52 | C |
| ATOM | 1993 | O | PRO | B | 151 | −64.441 | 40.470 | −19.831 | 1.00 | 37.58 | O |
| ATOM | 1994 | N | PRO | B | 152 | −66.316 | 41.637 | −20.374 | 1.00 | 37.11 | N |
| ATOM | 1995 | CA | PRO | B | 152 | −65.559 | 42.692 | −21.061 | 1.00 | 37.44 | C |
| ATOM | 1996 | CB | PRO | B | 152 | −66.655 | 43.592 | −21.640 | 1.00 | 37.17 | C |
| ATOM | 1997 | CG | PRO | B | 152 | −67.829 | 43.388 | −20.747 | 1.00 | 36.94 | C |
| ATOM | 1998 | CD | PRO | B | 152 | −67.746 | 41.975 | −20.244 | 1.00 | 36.96 | C |
| ATOM | 1999 | C | PRO | B | 152 | −64.645 | 43.483 | −20.110 | 1.00 | 37.58 | C |
| ATOM | 2000 | O | PRO | B | 152 | −64.975 | 43.598 | −18.927 | 1.00 | 38.39 | O |
| ATOM | 2001 | N | PRO | B | 153 | −63.500 | 44.010 | −20.609 | 1.00 | 37.41 | N |
| ATOM | 2002 | CA | PRO | B | 153 | −62.639 | 44.832 | −19.741 | 1.00 | 36.61 | C |
| ATOM | 2003 | CB | PRO | B | 153 | −61.422 | 45.146 | −20.625 | 1.00 | 37.35 | C |
| ATOM | 2004 | CG | PRO | B | 153 | −61.460 | 44.143 | −21.725 | 1.00 | 37.46 | C |
| ATOM | 2005 | CD | PRO | B | 153 | −62.913 | 43.855 | −21.954 | 1.00 | 37.84 | C |
| ATOM | 2006 | C | PRO | B | 153 | −63.338 | 46.117 | −19.293 | 1.00 | 35.57 | C |
| ATOM | 2007 | O | PRO | B | 153 | −64.119 | 46.690 | −20.057 | 1.00 | 36.69 | O |
| ATOM | 2008 | N | GLY | B | 154 | −63.051 | 46.546 | −18.065 | 1.00 | 33.68 | N |
| ATOM | 2009 | CA | GLY | B | 154 | −63.848 | 47.564 | −17.372 | 1.00 | 32.89 | C |
| ATOM | 2010 | C | GLY | B | 154 | −64.715 | 46.968 | −16.266 | 1.00 | 32.02 | C |
| ATOM | 2011 | O | GLY | B | 154 | −65.120 | 47.682 | −15.347 | 1.00 | 31.69 | O |
| ATOM | 2012 | N | THR | B | 155 | −65.006 | 45.665 | −16.364 | 1.00 | 30.87 | N |
| ATOM | 2013 | CA | THR | B | 155 | −65.732 | 44.910 | −15.331 | 1.00 | 29.73 | C |
| ATOM | 2014 | CB | THR | B | 155 | −65.847 | 43.414 | −15.729 | 1.00 | 29.76 | C |
| ATOM | 2015 | OG1 | THR | B | 155 | −66.660 | 43.299 | −16.903 | 1.00 | 29.56 | O |
| ATOM | 2016 | CG2 | THR | B | 155 | −66.649 | 42.550 | −14.619 | 1.00 | 29.83 | C |
| ATOM | 2017 | C | THR | B | 155 | −65.071 | 45.036 | −13.954 | 1.00 | 28.43 | C |
| ATOM | 2018 | O | THR | B | 155 | −63.843 | 46.739 | −13.850 | 1.00 | 28.99 | O |
| ATOM | 2019 | N | ARG | B | 156 | −65.901 | 47.775 | −12.911 | 1.00 | 26.96 | N |
| ATOM | 2020 | CA | ARG | B | 156 | −65.453 | 45.307 | −11.533 | 1.00 | 25.90 | C |
| ATOM | 2021 | CB | ARG | B | 156 | −65.807 | 46.739 | −11.114 | 1.00 | 25.70 | C |
| ATOM | 2022 | CG | ARG | B | 156 | −64.772 | 47.775 | −11.548 | 1.00 | 25.85 | C |
| ATOM | 2023 | CD | ARG | B | 156 | −65.391 | 49.085 | −12.022 | 1.00 | 25.66 | C |
| ATOM | 2024 | NE | ARG | B | 156 | −66.165 | 49.779 | −10.991 | 1.00 | 25.66 | N |
| ATOM | 2025 | CZ | ARG | B | 156 | −65.657 | 50.497 | −9.985 | 1.00 | 25.67 | C |
| ATOM | 2026 | NH1 | ARG | B | 156 | −64.340 | 50.632 | −9.815 | 1.00 | 25.32 | N |
| ATOM | 2027 | NH2 | ARG | B | 156 | −66.482 | 51.087 | −9.123 | 1.00 | 25.67 | N |
| ATOM | 2028 | C | ARG | B | 156 | −66.066 | 44.278 | −10.572 | 1.00 | 24.75 | C |
| ATOM | 2029 | O | ARG | B | 156 | −67.210 | 43.857 | −10.758 | 1.00 | 24.03 | O |
| ATOM | 2030 | N | VAL | B | 157 | −65.293 | 43.891 | −9.554 | 1.00 | 23.92 | N |
| ATOM | 2031 | CA | VAL | B | 157 | −65.694 | 42.872 | −8.567 | 1.00 | 23.76 | C |
| ATOM | 2032 | CB | VAL | B | 157 | −64.634 | 41.743 | −8.450 | 1.00 | 23.46 | C |
| ATOM | 2033 | CG1 | VAL | B | 157 | −65.207 | 40.536 | −7.714 | 1.00 | 23.68 | C |
| ATOM | 2034 | CG2 | VAL | B | 157 | −64.110 | 41.336 | −9.821 | 1.00 | 23.49 | C |
| ATOM | 2035 | C | VAL | B | 157 | −65.876 | 43.526 | −7.191 | 1.00 | 23.40 | C |
| ATOM | 2036 | O | VAL | B | 157 | −64.892 | 43.872 | −6.542 | 1.00 | 23.92 | O |
| ATOM | 2037 | N | ARG | B | 158 | −67.126 | 43.678 | −6.748 | 1.00 | 22.79 | N |
| ATOM | 2038 | CA | ARG | B | 158 | −67.440 | 44.304 | −5.453 | 1.00 | 22.33 | C |
| ATOM | 2039 | CB | ARG | B | 158 | −68.634 | 45.259 | −5.606 | 1.00 | 22.03 | C |
| ATOM | 2040 | CG | ARG | B | 158 | −69.081 | 45.965 | −4.322 | 1.00 | 21.99 | C |
| ATOM | 2041 | CD | ARG | B | 158 | −70.249 | 46.920 | −4.547 | 1.00 | 21.76 | C |
| ATOM | 2042 | NE | ARG | B | 158 | −70.033 | 47.799 | −5.695 | 1.00 | 21.91 | N |
| ATOM | 2043 | CZ | ARG | B | 158 | −69.331 | 48.936 | −5.694 | 1.00 | 22.09 | C |
| ATOM | 2044 | NH1 | ARG | B | 158 | −68.765 | 49.418 | −4.584 | 1.00 | 22.08 | N |
| ATOM | 2045 | NH2 | ARG | B | 158 | −69.207 | 49.614 | −6.830 | 1.00 | 22.02 | N |
| ATOM | 2046 | C | ARG | B | 158 | −67.733 | 43.253 | −4.374 | 1.00 | 22.03 | C |
| ATOM | 2047 | O | ARG | B | 158 | −68.409 | 42.262 | −4.648 | 1.00 | 22.03 | O |
| ATOM | 2048 | N | ALA | B | 159 | −57.228 | 43.493 | −3.161 | 1.00 | 21.29 | N |
| ATOM | 2049 | CA | ALA | B | 159 | −67.541 | 42.692 | −1.973 | 1.00 | 21.05 | C |
| ATOM | 2050 | CB | ALA | B | 159 | −66.275 | 42.062 | −1.412 | 1.00 | 21.06 | C |
| ATOM | 2051 | C | ALA | B | 159 | −68.227 | 43.558 | −0.905 | 1.00 | 20.94 | C |
| ATOM | 2052 | O | ALA | B | 159 | −67.767 | 44.664 | −0.606 | 1.00 | 20.32 | O |
| ATOM | 2053 | N | MET | B | 160 | −69.332 | 43.050 | −0.353 | 1.00 | 21.12 | N |
| ATOM | 2054 | CA | MET | B | 160 | −70.099 | 43.725 | 0.703 | 1.00 | 21.26 | C |
| ATOM | 2055 | CB | MET | B | 160 | −71.353 | 44.373 | 0.107 | 1.00 | 21.68 | C |
| ATOM | 2056 | CG | MET | B | 160 | −72.270 | 45.054 | 1.121 | 1.00 | 21.85 | C |
| ATOM | 2057 | SD | MET | B | 160 | −73.753 | 45.742 | 0.367 | 1.00 | 22.02 | S |
| ATOM | 2058 | CE | MET | B | 160 | −73.133 | 47.301 | −0.257 | 1.00 | 21.80 | C |
| ATOM | 2059 | C | MET | B | 160 | −70.577 | 42.719 | 1.775 | 1.00 | 21.25 | C |
| ATOM | 2060 | O | MET | B | 160 | −70.848 | 41.578 | 1.457 | 1.00 | 20.94 | O |
| ATOM | 2061 | N | ALA | B | 161 | −70.524 | 43.163 | 3.033 | 1.00 | 21.56 | N |
| ATOM | 2062 | CA | ALA | B | 161 | −71.020 | 42.357 | 4.156 | 1.00 | 22.03 | C |
| ATOM | 2063 | CB | ALA | B | 161 | −70.124 | 42.529 | 5.371 | 1.00 | 21.98 | C |
| ATOM | 2064 | C | ALA | B | 161 | −72.457 | 42.741 | 4.514 | 1.00 | 22.30 | C |
| ATOM | 2065 | O | ALA | B | 161 | −72.776 | 43.926 | 4.594 | 1.00 | 22.81 | O |
| ATOM | 2066 | N | ILE | B | 162 | −73.317 | 41.740 | 4.706 | 1.00 | 22.89 | N |
| ATOM | 2067 | CA | ILE | B | 162 | −74.639 | 41.936 | 5.324 | 1.00 | 23.12 | C |
| ATOM | 2068 | CB | ILE | B | 162 | −75.810 | 41.866 | 4.300 | 1.00 | 23.18 | C |
| ATOM | 2069 | CG1 | ILE | B | 162 | −76.102 | 40.432 | 3.815 | 1.00 | 23.38 | C |
| ATOM | 2070 | CD1 | ILE | B | 162 | −77.488 | 40.275 | 3.223 | 1.00 | 23.41 | C |
| ATOM | 2071 | CG2 | ILE | B | 162 | −75.534 | 42.778 | 3.110 | 1.00 | 22.95 | C |
| ATOM | 2072 | C | ILE | B | 162 | −74.834 | 40.916 | 6.445 | 1.00 | 23.48 | C |
| ATOM | 2073 | O | ILE | B | 162 | −72.262 | 39.824 | 6.399 | 1.00 | 23.42 | O |
| ATOM | 2074 | N | TYR | B | 163 | −75.626 | 41.280 | 7.453 | 1.00 | 23.86 | N |
| ATOM | 2075 | CA | TYR | B | 163 | −76.044 | 40.329 | 8.487 | 1.00 | 24.51 | C |
| ATOM | 2076 | CB | TYR | B | 163 | −76.631 | 41.047 | 9.709 | 1.00 | 23.78 | C |
| ATOM | 2077 | CG | TYR | B | 163 | −75.599 | 41.827 | 10.500 | 1.00 | 23.31 | C |
| ATOM | 2078 | CD1 | TYR | B | 163 | −74.595 | 41.165 | 11.212 | 1.00 | 22.88 | C |
| ATOM | 2079 | CE1 | TYR | B | 163 | −73.641 | 41.867 | 11.935 | 1.00 | 22.86 | C |
| ATOM | 2080 | CZ | TYR | B | 163 | −73.682 | 43.255 | 11.954 | 1.00 | 22.88 | C |
| ATOM | 2081 | OH | TYR | B | 163 | −72.739 | 43.945 | 12.688 | 1.00 | 22.71 | O |
| ATOM | 2082 | CE2 | TYR | B | 163 | −74.665 | 43.937 | 11.255 | 1.00 | 22.88 | C |
| ATOM | 2083 | CD2 | TYR | B | 163 | −75.618 | 43.222 | 10.535 | 1.00 | 23.03 | C |
| ATOM | 2084 | C | TYR | B | 163 | −77.065 | 39.368 | 7.883 | 1.00 | 25.80 | C |
| ATOM | 2085 | O | TYR | B | 163 | −77.957 | 39.791 | 7.156 | 1.00 | 26.15 | O |
| ATOM | 2086 | N | LYS | B | 164 | −76.915 | 38.080 | 8.176 | 1.00 | 27.97 | N |
| ATOM | 2087 | CA | LYS | B | 164 | −77.781 | 37.047 | 7.612 | 1.00 | 29.55 | C |
| ATOM | 2088 | CB | LYS | B | 164 | −77.102 | 35.675 | 7.700 | 1.00 | 29.84 | C |
| ATOM | 2089 | CO | LYS | B | 164 | −77.597 | 34.688 | 6.656 | 1.00 | 30.34 | C |
| ATOM | 2090 | CD | LYS | B | 164 | −77.201 | 33.254 | 6.980 | 1.00 | 30.70 | C |
| ATOM | 2091 | CE | LYS | B | 164 | −78.104 | 32.259 | 6.265 | 1.00 | 31.12 | C |
| ATOM | 2092 | NZ | LYS | B | 164 | −77.837 | 30.856 | 6.684 | 1.00 | 31.52 | N |
| ATOM | 2093 | C | LYS | B | 164 | −79.127 | 37.026 | 8.338 | 1.00 | 30.83 | C |
| ATOM | 2094 | O | LYS | B | 164 | −80.183 | 36.986 | 7.703 | 1.00 | 30.58 | O |
| ATOM | 2095 | N | GLN | B | 165 | −79.051 | 37.040 | 9.671 | 1.00 | 32.64 | N |
| ATOM | 2096 | CA | GLN | B | 165 | −80.200 | 37.186 | 10.594 | 1.00 | 33.67 | C |
| ATOM | 2097 | CB | GLN | B | 165 | −79.724 | 37.642 | 11.989 | 1.00 | 34.21 | C |
| ATOM | 2098 | CG | GLN | B | 165 | −78.644 | 36.789 | 12.663 | 1.00 | 34.86 | C |
| ATOM | 2099 | CD | GLN | B | 165 | −77.218 | 37.264 | 12.394 | 1.00 | 34.99 | C |
| ATOM | 2100 | OE1 | GLN | B | 165 | −76.921 | 38.457 | 12.455 | 1.00 | 34.60 | O |
| ATOM | 2101. | NE2 | GLN | B | 165 | −76.330 | 36.321 | 12.089 | 1.00 | 35.10 | N |
| ATOM | 2102 | C | GLN | B | 165 | −81.239 | 38.203 | 10.104 | 1.00 | 34.06 | C |
| ATOM | 2103 | O | GLN | B | 165 | −80.675 | 39.331 | 9.773 | 1.00 | 35.00 | O |
| ATOM | 2104 | N | SER | B | 166 | −82.519 | 37.819 | 10.088 | 1.00 | 34.38 | N |
| ATOM | 2105 | CA | SER | B | 166 | −83.585 | 38.662 | 9.514 | 1.00 | 34.51 | C |
| ATOM | 2106 | CB | SER | B | 166 | −84.929 | 37.920 | 9.494 | 1.00 | 34.83 | C |
| ATOM | 2107 | OG | SER | B | 166 | −85.489 | 37.823 | 10.793 | 1.00 | 35.33 | O |
| ATOM | 2108 | C | SER | B | 166 | −83.739 | 40.015 | 10.223 | 1.00 | 34.45 | C |
| ATOM | 2109 | O | SER | B | 166 | −83.779 | 41.060 | 9.569 | 1.00 | 34.69 | O |
| ATOM | 2110 | N | GLN | B | 167 | −83.808 | 39.986 | 11.554 | 1.00 | 34.47 | N |
| ATOM | 2111 | CA | GLN | B | 167 | −83.928 | 41.218 | 12.363 | 1.00 | 34.44 | C |
| ATOM | 2112 | CB | GLN | B | 167 | −84.406 | 40.911 | 13.800 | 1.00 | 35.21 | C |
| ATOM | 2113 | CG | GLN | B | 167 | −83.351 | 40.427 | 14.798 | 1.00 | 35.89 | C |
| ATOM | 2114 | CD | GLN | B | 167 | −82.808 | 39.044 | 14.492 | 1.00 | 36.36 | C |
| ATOM | 2115 | OE1 | GLN | B | 167 | −83.523 | 38.179 | 13.981 | 1.00 | 37.62 | O |
| ATOM | 2116 | NE2 | GLN | B | 167 | −81.541 | 38.823 | 14.812 | 1.00 | 36.90 | N |
| ATOM | 2117 | C | GLN | B | 167 | −82.682 | 42.128 | 12.396 | 1.00 | 33.69 | C |
| ATOM | 2118 | O | GLN | B | 167 | −82.765 | 43.239 | 12.927 | 1.00 | 34.07 | O |
| ATOM | 2119 | N | HIS | B | 168 | −81.545 | 41.660 | 11.871 | 1.00 | 31.96 | N |
| ATOM | 2120 | C | HIS | B | 168 | −80.341 | 42.493 | 11.714 | 1.00 | 31.00 | C |
| ATOM | 2121 | CB | HIS | B | 168 | −79.132 | 41.819 | 12.379 | 1.00 | 31.49 | C |
| ATOM | 2122 | CO | HIS | B | 168 | −79.299 | 41.573 | 13.847 | 1.00 | 31.99 | C |
| ATOM | 2123 | ND1 | HIS | B | 168 | −79.837 | 42.508 | 14.705 | 1.00 | 32.23 | N |
| ATOM | 2124 | CE1 | HIS | B | 168 | −79.845 | 42.022 | 15.934 | 1.00 | 32.66 | C |
| ATOM | 2125 | NE2 | HIS | B | 168 | −79.324 | 40.810 | 15.906 | 1.00 | 32.72 | N |
| ATOM | 2126 | 002 | HIS | B | 168 | −78.967 | 40.508 | 14.614 | 1.00 | 32.40 | C |
| ATOM | 2127 | C | HIS | B | 168 | −79.980 | 42.843 | 10.254 | 1.00 | 29.99 | C |
| ATOM | 2128 | O | HIS | B | 168 | −79.043 | 43.614 | 10.041 | 1.00 | 29.40 | O |
| ATOM | 2129 | N | MET | B | 169 | −80.709 | 42.311 | 9.262 | 1.00 | 28.67 | N |
| ATOM | 2130 | CA | MET | B | 169 | −80.392 | 42.544 | 7.832 | 1.00 | 28.13 | C |
| ATOM | 2131 | CB | MET | B | 169 | −81.417 | 41.860 | 6.905 | 1.00 | 29.32 | C |
| ATOM | 2132 | CG | MET | B | 169 | −81.270 | 40.350 | 6.790 | 1.00 | 30.37 | C |
| ATOM | 2133 | SD | MET | B | 169 | −81.826 | 39.662 | 5.217 | 1.00 | 32.48 | S |
| ATOM | 2134 | CE | MET | B | 169 | −83.593 | 39.942 | 5.334 | 1.00 | 32.32 | C |
| ATOM | 2135 | C | MET | B | 169 | −80.729 | 44.024 | 7.433 | 1.00 | 26.06 | C |
| ATOM | 2136 | O | MET | B | 169 | −79.466 | 44.374 | 6.574 | 1.00 | 24.86 | O |
| ATOM | 2137 | N | THR | B | 170 | −81.093 | 44.879 | 8.052 | 1.00 | 24.26 | N |
| ATOM | 2138 | CA | THR | B | 170 | −81.059 | 46.323 | 7.786 | 1.00 | 23.49 | C |
| ATOM | 2139 | CB | THR | B | 170 | −82.370 | 46.999 | 8.222 | 1.00 | 22.97 | C |
| ATOM | 2140 | OG1 | THR | B | 170 | −82.676 | 46.620 | 7.565 | 1.00 | 22.63 | O |
| ATOM | 2141 | CG2 | THR | B | 170 | −83.512 | 46.585 | 7.307 | 1.00 | 22.92 | C |
| ATOM | 2142 | C | THR | B | 170 | −79.878 | 47.061 | 8.442 | 1.00 | 23.05 | C |
| ATOM | 2143 | O | THR | B | 170 | −79.589 | 48.198 | 8.067 | 1.00 | 23.60 | O |
| ATOM | 2144 | N | GLU | B | 171 | −79.207 | 46.431 | 9.410 | 1.00 | 22.48 | N |
| ATOM | 2145 | CA | GLU | B | 171 | −78.050 | 47.040 | 10.081 | 1.00 | 22.13 | C |
| ATOM | 2146 | CB | GLU | B | 171 | −77.754 | 46.346 | 11.409 | 1.00 | 22.66 | C |
| ATOM | 2147 | CG | GLU | B | 171 | −78.893 | 46.449 | 12.406 | 1.00 | 23.20 | C |
| ATOM | 2148 | CD | GLU | B | 171 | −78.696 | 45.581 | 13.626 | 1.00 | 23.58 | C |
| ATOM | 2149 | OE1 | GLU | B | 171 | −79.694 | 44.984 | 14.077 | 1.00 | 24.73 | O |
| ATOM | 2150 | OE2 | GLU | B | 171 | −77.559 | 45.495 | 14.137 | 1.00 | 23.96 | O |
| ATOM | 2151 | C | GLU | B | 171 | −76.811 | 46.975 | 9.204 | 1.00 | 21.41 | C |
| ATOM | 2152 | O | GLU | B | 171 | −76.485 | 45.921 | 8.651 | 1.00 | 21.09 | O |
| ATOM | 2153 | N | VAL | B | 172 | −76.116 | 48.102 | 9.097 | 1.00 | 20.67 | N |
| ATOM | 2154 | CA | VAI | B | 172 | −74.896 | 48.183 | 8.308 | 1.00 | 20.16 | C |
| ATOM | 2155 | CB | VAL | B | 172 | −74.437 | 49.645 | 8.102 | 1.00 | 20.31 | C |
| ATOM | 2156 | CG1 | VAL | B | 172 | −73.061 | 49.702 | 7.440 | 1.00 | 20.38 | C |
| ATOM | 2157 | CG2 | VAL | B | 172 | −75.460 | 50.422 | 7.280 | 1.00 | 20.37 | C |
| ATOM | 2158 | C | VAL | B | 172 | −73.822 | 47.400 | 9.046 | 1.00 | 19.58 | C |
| ATOM | 2159 | O | VAL | B | 172 | −73.621 | 47.604 | 10.241 | 1.00 | 18.60 | O |
| ATOM | 2160 | N | VAL | B | 173 | −73.158 | 46.494 | 8.330 | 1.00 | 19.02 | N |
| ATOM | 2161 | CA | VAL | B | 173 | −72.045 | 45.736 | 8.881 | 1.00 | 19.13 | C |
| ATOM | 2162 | CB | VAL | B | 173 | −71.742 | 44.449 | 8.075 | 1.00 | 18.97 | C |
| ATOM | 2163 | CG1 | VAL | B | 173 | −70.607 | 43.663 | 8.723 | 1.00 | 19.10 | C |
| ATOM | 2164 | CG2 | VAL | B | 173 | −72.981 | 43.568 | 7.955 | 1.00 | 19.03 | C |
| ATOM | 2165 | C | VAL | B | 173 | −70.831 | 46.666 | 8.845 | 1.00 | 19.36 | C |
| ATOM | 2166 | O | VAL | B | 173 | −70.552 | 47.294 | 7.823 | 1.00 | 18.90 | O |
| ATOM | 2167 | N | ARG | B | 174 | −70.125 | 46.758 | 9.966 | 1.00 | 20.09 | N |
| ATOM | 2168 | CA | ARG | B | 174 | −68.877 | 47.510 | 10.031 | 1.00 | 20.84 | C |
| ATOM | 2169 | CB | ARG | B | 174 | −69.141 | 49.023 | 10.168 | 1.00 | 20.77 | C |
| ATOM | 2170 | CG | ARG | B | 174 | −69.644 | 49.507 | 11.523 | 1.00 | 20.68 | C |
| ATOM | 2171 | CD | ARG | B | 174 | −71.053 | 49.032 | 11.838 | 1.00 | 20.80 | C |
| ATOM | 2172 | NE | ARG | B | 174 | −71.619 | 49.776 | 12.961 | 1.00 | 20.95 | N |
| ATOM | 2173 | CZ | ARG | B | 174 | −72.917 | 49.865 | 13.260 | 1.00 | 21.08 | C |
| ATOM | 2174 | NH1 | ARG | B | 174 | −73.847 | 49.258 | 12.518 | 1.00 | 21.29 | N |
| ATOM | 2175 | NH2 | ARG | B | 174 | −73.286 | 50.579 | 14.319 | 1.00 | 21.05 | N |
| ATOM | 2176 | C | ARG | B | 174 | −68.002 | 46.989 | 11.161 | 1.00 | 22.09 | C |
| ATOM | 2177 | O | ARG | B | 174 | −66.395 | 46.079 | 11.893 | 1.00 | 21.80 | O |
| ATOM | 2178 | N | ARG | B | 175 | −66.813 | 47.570 | 11.277 | 1.00 | 23.78 | N |
| ATOM | 2179 | CA | ARG | B | 175 | −65.884 | 47.258 | 12.361 | 1.00 | 25.96 | C |
| ATOM | 2180 | CB | ARG | B | 175 | −64.519 | 47.880 | 12.071 | 1.00 | 25.90 | C |
| ATOM | 2181 | CG | ARG | B | 175 | −63.765 | 47.172 | 10.965 | 1.00 | 26.12 | C |
| ATOM | 2182 | CD | ARG | B | 175 | −62.297 | 46.991 | 11.307 | 1.00 | 26.60 | C |
| ATOM | 2183 | NE | ARG | B | 175 | −61.461 | 48.112 | 10.901 | 1.00 | 27.01 | N |
| ATOM | 2184 | CZ | ARG | B | 175 | −60.165 | 48.238 | 11.190 | 1.00 | 26.97 | C |
| ATOM | 2185 | NH1 | ARG | B | 175 | −59.510 | 49.311 | 10.767 | 1.00 | 26.97 | N |
| ATOM | 2186 | NH2 | ARG | B | 175 | −59.517 | 47.314 | 11.900 | 1.00 | 26.85 | N |
| ATOM | 2187 | C | ARG | B | 175 | −66.360 | 47.735 | 13.730 | 1.00 | 27.77 | C |
| ATOM | 2188 | O | ARG | B | 175 | −67.184 | 48.643 | 13.837 | 1.00 | 27.78 | O |
| ATOM | 2189 | N | CYS | B | 176 | −65.803 | 47.122 | 14.772 | 1.00 | 29.68 | N |
| ATOM | 2190 | CA | CYS | B | 176 | −66.109 | 47.494 | 16.145 | 1.00 | 31.43 | C |
| ATOM | 2191 | CB | CYS | B | 176 | −65.789 | 46.340 | 17.096 | 1.00 | 32.26 | C |
| ATOM | 2192 | SG | CYS | B | 176 | −64.040 | 46.084 | 17.478 | 1.00 | 32.07 | S |
| ATOM | 2193 | C | CYS | B | 176 | −65.319 | 48.761 | 16.516 | 1.00 | 32.50 | C |
| ATOM | 2194 | O | CYS | B | 176 | −64.336 | 49.091 | 15.845 | 1.00 | 32.88 | O |
| ATOM | 2195 | N | PRO | B | 177 | −65.755 | 49.483 | 17.569 | 1.00 | 33.52 | N |
| ATOM | 2196 | CA | PRO | B | 177 | −65.073 | 50.696 | 18.044 | 1.00 | 34.01 | C |
| ATOM | 2197 | CB | PRO | B | 177 | −65.828 | 51.021 | 19.331 | 1.00 | 34.15 | C |
| ATOM | 2198 | CG | PRO | B | 177 | −67.209 | 50.540 | 19.077 | 1.00 | 33.88 | C |
| ATOM | 2199 | CD | PRO | B | 177 | −67.067 | 49.311 | 18.229 | 1.00 | 33.71 | C |
| ATOM | 2200 | C | PRO | B | 177 | −63.566 | 50.570 | 18.334 | 1.00 | 34.75 | C |
| ATOM | 2201 | O | PRO | B | 177 | −62.798 | 51.453 | 17.953 | 1.00 | 34.66 | O |
| ATOM | 2202 | N | HIS | B | 178 | −63.164 | 49.499 | 19.018 | 1.00 | 35.32 | N |
| ATOM | 2203 | CA | HIS | B | 178 | −61.749 | 49.257 | 19.364 | 1.00 | 36.20 | C |
| ATOM | 2204 | CB | HIS | B | 178 | −61.622 | 48.043 | 20.306 | 1.00 | 36.25 | C |
| ATOM | 2205 | CG | HIS | B | 178 | −60.219 | 47.549 | 20.488 | 1.00 | 36.56 | C |
| ATOM | 2206 | ND1 | HIS | B | 178 | −59.289 | 48.208 | 21.263 | 1.00 | 36.70 | N |
| ATOM | 2207 | CE1 | HIS | B | 178 | −58.150 | 47.539 | 21.244 | 1.00 | 36.74 | C |
| ATOM | 2208 | NH2 | HIS | B | 178 | −58.306 | 46.471 | 20.482 | 1.00 | 36.89 | N |
| ATOM | 2209 | CD2 | HIS | B | 178 | −59.592 | 46.452 | 20.000 | 1.00 | 36.57 | C |
| ATOM | 2210 | C | HIS | B | 178 | −60.812 | 49.110 | 18.149 | 1.00 | 37.06 | C |
| ATOM | 2211 | O | HIS | B | 178 | −59.870 | 49.893 | 18.006 | 1.00 | 38.21 | O |
| ATOM | 2212 | N | HIS | B | 179 | −61.067 | 48.123 | 17.286 | 1.00 | 38.25 | N |
| ATOM | 2213 | CA | HIS | B | 179 | −60.190 | 47.856 | 16.125 | 1.00 | 39.14 | C |
| ATOM | 2214 | CB | HIS | B | 179 | −60.494 | 46.503 | 15.487 | 1.00 | 37.91 | C |
| ATOM | 2215 | CG | HIS | B | 179 | −60.186 | 45.333 | 16.368 | 1.00 | 37.00 | C |
| ATOM | 2216 | ND1 | HIS | B | 179 | −61.146 | 44.421 | 16.752 | 1.00 | 36.10 | N |
| ATOM | 2217 | CE1 | HIS | B | 179 | −60.590 | 43.500 | 17.521 | 1.00 | 36.36 | C |
| ATOM | 2218 | NE2 | HIS | B | 179 | −59.306 | 43.783 | 17.649 | 1.00 | 36.17 | N |
| ATOM | 2219 | CD2 | HIS | B | 179 | −59.027 | 44.924 | 16.936 | 1.00 | 36.80 | C |
| ATOM | 2220 | C | HIS | B | 179 | −60.227 | 48.944 | 15.039 | 1.00 | 40.95 | C |
| ATOM | 2221 | O | HIS | B | 179 | −59.304 | 49.025 | 14.224 | 1.00 | 41.56 | O |
| ATOM | 2222 | N | GLU | B | 180 | −61.290 | 49.751 | 15.013 | 1.00 | 43.65 | N |
| ATOM | 2223 | CA | GLU | B | 180 | −61.308 | 50.989 | 14.225 | 1.00 | 44.96 | C |
| ATOM | 2224 | CB | GLU | B | 180 | −62.666 | 51.690 | 14.349 | 1.00 | 44.78 | C |
| ATOM | 2225 | CD | GLU | B | 180 | −62.800 | 52.988 | 13.553 | 1.00 | 44.38 | C |
| ATOM | 2226 | CG | GLU | B | 180 | −64.066 | 53.761 | 13.882 | 1.00 | 44.13 | C |
| ATOM | 2227 | OE1 | GLU | B | 180 | −65.017 | 53.164 | 14.428 | 1.00 | 43.63 | O |
| ATOM | 2228 | OE2 | GLU | B | 180 | −64.110 | 54.975 | 13.589 | 1.00 | 43.02 | O |
| ATOM | 2229 | C | GLU | B | 180 | −60.204 | 51.927 | 14.712 | 1.00 | 47.08 | C |
| ATOM | 2230 | O | GLU | B | 180 | −59.379 | 52.386 | 13.922 | 1.00 | 48.26 | O |
| ATOM | 2231 | N | ARG | B | 181 | −60.200 | 52.187 | 16.019 | 1.00 | 49.23 | N |
| ATOM | 2232 | CA | ARG | B | 181 | −59.236 | 53.102 | 16.645 | 1.00 | 51.00 | C |
| ATOM | 2233 | CB | ARG | B | 181 | −59.775 | 53.604 | 17.988 | 1.00 | 51.65 | C |
| ATOM | 2234 | CG | ARG | B | 181 | −61.099 | 54.342 | 17.858 | 1.00 | 51.98 | C |
| ATOM | 2235 | CD | ARG | B | 181 | −61.492 | 55.073 | 19.133 | 1.00 | 52.29 | C |
| ATOM | 2236 | NE | ARG | B | 181 | −60.991 | 56.446 | 19.156 | 1.00 | 52.62 | O |
| ATOM | 2237 | CZ | ARG | B | 181 | −61.493 | 57.466 | 18.449 | 1.00 | 52.65 | N |
| ATOM | 2238 | NH1 | ARG | B | 181 | −62.530 | 57.298 | 17.624 | 1.00 | 52.51 | N |
| ATOM | 2239 | NH2 | ARG | B | 181 | −60.943 | 58.672 | 18.560 | 1.00 | 52.59 | N |
| ATOM | 2240 | C | ARG | B | 181 | −57.829 | 52.510 | 16.820 | 1.00 | 51.83 | C |
| ATOM | 2241 | O | ARG | B | 181 | −56.890 | 53.244 | 17.139 | 1.00 | 52.18 | O |
| ATOM | 2242 | N | CYS | B | 182 | −57.687 | 51.196 | 16.632 | 1.00 | 53.07 | N |
| ATOM | 2243 | CA | CYS | B | 182 | −56.369 | 50.566 | 16.498 | 1.00 | 53.83 | C |
| ATOM | 2244 | CO | CYS | B | 182 | −56.472 | 49.041 | 16.591 | 1.00 | 54.36 | C |
| ATOM | 2245 | SG | CYS | B | 182 | −56.941 | 48.431 | 18.225 | 1.00 | 55.30 | S |
| ATOM | 2246 | C | CYS | B | 182 | −55.723 | 50.955 | 15.169 | 1.00 | 54.07 | C |
| ATOM | 2247 | O | CYS | B | 182 | −56.414 | 51.141 | 14.163 | 1.00 | 53.80 | O |
| ATOM | 2248 | N | SER | B | 183 | −54.397 | 51.062 | 15.178 | 1.00 | 54.36 | N |
| ATOM | 2249 | CA | SER | B | 183 | −53.631 | 51.499 | 14.010 | 1.00 | 54.99 | C |
| ATOM | 2250 | CB | SER | B | 183 | −52.282 | 52.065 | 14.460 | 1.00 | 54.85 | C |
| ATOM | 2251 | OG | SER | B | 183 | −51.493 | 52.484 | 13.360 | 1.00 | 54.33 | O |
| ATOM | 2252 | C | SER | B | 183 | −53.430 | 50.343 | 13.022 | 1.00 | 55.86 | C |
| ATOM | 2253 | O | SER | B | 183 | −52.569 | 49.484 | 13.231 | 1.00 | 55.82 | O |
| ATOM | 2254 | N | ASP | B | 184 | −54.231 | 50.340 | 11.952 | 1.00 | 56.61 | N |
| ATOM | 2255 | CA | ASP | B | 184 | −54.231 | 49.283 | 10.927 | 1.00 | 56.80 | C |
| ATOM | 2256 | CB | ASP | B | 184 | −55.490 | 48.432 | 11.039 | 1.00 | 57.36 | C |
| ATOM | 2257 | CG | ASP | B | 184 | −55.691 | 47.839 | 12.423 | 1.00 | 57.78 | C |
| ATOM | 2258 | OD1 | ASP | B | 184 | −54.707 | 47.360 | 13.025 | 1.00 | 58.65 | O |
| ATOM | 2259 | OD2 | ASP | B | 184 | −56.843 | 47.844 | 12.906 | 1.00 | 59.09 | O |
| ATOM | 2260 | C | ASP | B | 184 | −54.107 | 49.835 | 9.494 | 1.00 | 56.76 | C |
| ATOM | 2261 | O | ASP | B | 184 | −54.417 | 49.123 | 8.536 | 1.00 | 56.99 | O |
| ATOM | 2262 | N | SER | B | 185 | −53.650 | 51.078 | 9.340 | 1.00 | 56.43 | N |
| ATOM | 2263 | CA | SER | B | 185 | −53.706 | 51.763 | 8.043 | 1.00 | 55.96 | C |
| ATOM | 2264 | CB | SER | B | 185 | −53.582 | 53.281 | 8.226 | 1.00 | 56.03 | C |
| ATOM | 2265 | OG | SER | B | 185 | −53.856 | 53.970 | 7.016 | 1.00 | 56.45 | O |
| ATOM | 2266 | C | SER | B | 185 | −52.641 | 51.274 | 7.054 | 1.00 | 55.27 | C |
| ATOM | 2267 | O | SER | B | 185 | −51.443 | 51.506 | 7.245 | 1.00 | 55.53 | O |
| ATOM | 2268 | N | ASP | B | 186 | −53.097 | 50.587 | 6.006 | 1.00 | 54.07 | N |
| ATOM | 2269 | CA | ASP | B | 186 | −52.297 | 50.389 | 4.790 | 1.00 | 52.89 | C |
| ATOM | 2270 | CB | ASP | B | 186 | −52.755 | 49.142 | 4.003 | 1.00 | 53.24 | C |
| ATOM | 2271 | CG | ASP | B | 186 | −54.162 | 49.274 | 3.416 | 1.00 | 54.05 | C |
| ATOM | 2272 | OD1 | ASP | B | 186 | −54.969 | 50.084 | 3.922 | 1.00 | 54.94 | O |
| ATOM | 2273 | OD2 | ASP | B | 186 | −54.462 | 48.545 | 2.445 | 1.00 | 53.48 | O |
| ATOM | 2274 | C | ASP | B | 186 | −52.313 | 51.647 | 3.901 | 1.00 | 50.82 | C |
| ATOM | 2275 | O | ASP | B | 186 | −51.536 | 51.739 | 2.947 | 1.00 | 51.77 | O |
| ATOM | 2276 | N | GLY | B | 187 | −53.210 | 52.592 | 4.209 | 1.00 | 48.20 | N |
| ATOM | 2277 | CA | GLY | B | 187 | −53.261 | 53.899 | 3.550 | 1.00 | 45.78 | C |
| ATOM | 2278 | C | GLY | B | 187 | −54.526 | 54.102 | 2.738 | 1.00 | 43.20 | C |
| ATOM | 2279 | O | GLY | B | 187 | −55.020 | 55.227 | 2.630 | 1.00 | 43.11 | O |
| ATOM | 2280 | N | LEU | B | 188 | −55.051 | 53.015 | 2.175 | 1.00 | 39.45 | N |
| ATOM | 2281 | CA | LEU | B | 188 | −56.179 | 53.070 | 1.243 | 1.00 | 37.31 | C |
| ATOM | 2282 | CB | LEU | B | 188 | −55.949 | 52.081 | 0.091 | 1.00 | 37.07 | C |
| ATOM | 2283 | CG | LEU | B | 188 | −54.540 | 52.044 | −0.526 | 1.00 | 37.12 | C |
| ATOM | 2284 | CD1 | LEU | B | 188 | −54.533 | 51.172 | −1.774 | 1.00 | 37.28 | C |
| ATOM | 2285 | CD2 | LEU | B | 188 | −54.017 | 53.439 | −0.843 | 1.00 | 36.93 | C |
| ATOM | 2286 | C | LEU | B | 188 | −57.537 | 52.804 | 1.906 | 1.00 | 35.05 | C |
| ATOM | 2287 | O | LEU | B | 188 | −56.523 | 53.470 | 1.576 | 1.00 | 35.89 | O |
| ATOM | 2288 | N | ALA | B | 189 | −57.590 | 51.845 | 2.833 | 1.00 | 31.82 | N |
| ATOM | 2289 | CA | ALA | B | 189 | −56.860 | 51.399 | 3.422 | 1.00 | 29.80 | C |
| ATOM | 2290 | CB | ALA | B | 189 | −56.695 | 50.029 | 4.058 | 1.00 | 29.51 | C |
| ATOM | 2291 | C | ALA | B | 189 | −59.406 | 52.383 | 4.461 | 1.00 | 27.73 | C |
| ATOM | 2292 | O | ALA | B | 189 | −58.643 | 52.871 | 5.294 | 1.00 | 26.91 | O |
| ATOM | 2293 | N | PRO | B | 190 | −60.728 | 52.669 | 4.422 | 1.00 | 26.10 | N |
| ATOM | 2294 | CA | PRO | B | 190 | −61−350 | 53.415 | 5.521 | 1.00 | 25.07 | C |
| ATOM | 2295 | CB | PRO | B | 190 | −62.602 | 53.597 | 5.063 | 1.00 | 25.16 | C |
| ATOM | 2296 | CG | PRO | B | 190 | −62.800 | 53.353 | 3.600 | 1.00 | 25.44 | C |
| ATOM | 2297 | CD | PRO | B | 190 | −61.683 | 52.403 | 3.329 | 1.00 | 25.45 | C |
| ATOM | 2298 | C | PRO | B | 190 | −61.313 | 52.604 | 6.821 | 1.00 | 24.27 | C |
| ATOM | 2299 | O | PRO | B | 190 | −61.541 | 51.394 | 6.772 | 1.00 | 23.45 | O |
| ATOM | 2300 | N | PRO | B | 191 | −61.041 | 53.257 | 7.974 | 1.00 | 23.61 | N |
| ATOM | 2301 | CA | PRO | B | 191 | −60.932 | 52.515 | 9.247 | 1.00 | 23.21 | C |
| ATOM | 2302 | CB | PRO | B | 191 | −60.543 | 53.598 | 10.270 | 1.00 | 23.38 | C |
| ATOM | 2303 | CG | PRO | B | 191 | −60.608 | 54.905 | 9.612 | 1.00 | 23.42 | C |
| ATOM | 2304 | CD | PRO | B | 191 | −60.714 | 54.687 | 8.138 | 1.00 | 23.61 | C |
| ATOM | 2305 | C | PRO | B | 191 | −62.187 | 51.749 | 9.718 | 1.00 | 22.37 | C |
| ATOM | 2306 | O | PRO | B | 191 | −62.053 | 50.826 | 10.513 | 1.00 | 22.53 | O |
| ATOM | 2307 | N | GLN | B | 192 | −63.375 | 52.125 | 9.242 | 1.00 | 22.15 | N |
| ATOM | 2308 | CA | GLN | B | 192 | −64.626 | 51.443 | 9.615 | 1.00 | 22.09 | C |
| ATOM | 2309 | CB | GLN | B | 192 | −65.814 | 52.397 | 9.503 | 1.00 | 22.48 | C |
| ATOM | 2310 | CG | GLN | B | 192 | −65.716 | 53.598 | 10.427 | 1.00 | 23.40 | C |
| ATOM | 2311 | CD | GLN | B | 192 | −66.668 | 54.571 | 10.266 | 1.00 | 23.79 | C |
| ATOM | 2312 | OE1 | GLN | B | 192 | −67.603 | 54.545 | 9.273 | 1.00 | 24.27 | O |
| ATOM | 2313 | NE2 | GLN | B | 192 | −67.026 | 55.444 | 11.247 | 1.00 | 24.33 | N |
| ATOM | 2314 | C | GLN | B | 192 | −64.942 | 50.184 | 8.806 | 1.00 | 21.25 | C |
| ATOM | 2315 | O | GLN | B | 192 | −65.709 | 49.343 | 9.277 | 1.00 | 21.44 | O |
| ATOM | 2316 | N | HIS | B | 193 | −64.378 | 50.058 | 7.602 | 1.00 | 20.18 | N |
| ATOM | 2317 | CA | HIS | B | 193 | −64.678 | 48.918 | 6.721 | 1.00 | 19.65 | C |
| ATOM | 2318 | CB | HIS | B | 193 | −64.085 | 49.125 | 5.319 | 1.00 | 19.51 | C |
| ATOM | 2319 | CG | HIS | B | 193 | −64.872 | 50.069 | 4.465 | 1.00 | 19.70 | C |
| ATOM | 2320 | ND1 | HIS | B | 193 | −65.152 | 48.816 | 3.139 | 1.00 | 19.15 | N |
| ATOM | 2321 | CE1 | HIS | B | 193 | −65.680 | 50.815 | 2.645 | 1.00 | 19.34 | C |
| ATOM | 2322 | NE2 | HIS | B | 193 | −66.068 | 51.696 | 3.606 | 1.00 | 19.27 | N |
| ATOM | 2323 | CD2 | HIS | B | 193 | −65.460 | 51.254 | 4.754 | 1.00 | 19.42 | C |
| ATOM | 2324 | C | HIS | B | 193 | −64.195 | 47.577 | 7.290 | 1.00 | 19.22 | C |
| ATOM | 2325 | O | HIS | B | 193 | −63.026 | 47.424 | 7.639 | 1.00 | 18.63 | O |
| ATOM | 2326 | N | LEU | B | 194 | −65.114 | 46.618 | 7.374 | 1.00 | 19.13 | N |
| ATOM | 2327 | CA | LEU | B | 194 | −64.790 | 45.421 | 7.750 | 1.00 | 18.79 | C |
| ATOM | 2328 | CB | LEU | B | 194 | −66.076 | 44.428 | 7.931 | 1.00 | 18.88 | C |
| ATOM | 2329 | CG | LEU | B | 194 | −65.950 | 42.944 | 8.296 | 1.00 | 18.66 | C |
| ATOM | 2330 | CBS | LEU | B | 194 | −65.305 | 42.781 | 9.658 | 1.00 | 18.62 | C |
| ATOM | 2331 | CO2 | LEU | B | 194 | −67.316 | 42.276 | 8.255 | 1.00 | 18.56 | C |
| ATOM | 2332 | C | LEU | B | 194 | −63.901 | 44.566 | 6.700 | 1.00 | 18.93 | C |
| ATOM | 2333 | O | LEU | B | 194 | −62.882 | 43.970 | 7.045 | 1.00 | 18.95 | O |
| ATOM | 2334 | N | ILE | B | 195 | −64.291 | 44.672 | 5.429 | 1.00 | 18.81 | N |
| ATOM | 2335 | CA | ILE | B | 195 | −63.618 | 43.959 | 4.334 | 1.00 | 18.92 | C |
| ATOM | 2336 | CB | ILE | B | 195 | −64.637 | 43.379 | 3.316 | 1.00 | 18.46 | C |
| ATOM | 2337 | CG1 | ILE | B | 195 | −65.746 | 42.598 | 4.045 | 1.00 | 18.26 | C |
| ATOM | 2338 | CD1 | ILE | B | 195 | −66.846 | 42.059 | 3.150 | 1.00 | 18.02 | C |
| ATOM | 2339 | CG2 | ILE | B | 195 | −63.924 | 42.477 | 2.303 | 1.00 | 18.45 | C |
| ATOM | 2340 | C | ILE | B | 195 | −62.628 | 44.870 | 3.596 | 1.00 | 19.25 | C |
| ATOM | 2341 | O | ILE | B | 195 | −63.004 | 45.942 | 3.122 | 1.00 | 19.63 | O |
| ATOM | 2342 | N | ARG | B | 196 | −61.376 | 44.415 | 3.503 | 1.00 | 20.33 | N |
| ATOM | 2343 | CA | ARG | B | 196 | −60.318 | 45.036 | 2.685 | 1.00 | 21.07 | C |
| ATOM | 2344 | CB | ARG | B | 196 | −59.125 | 45.413 | 3.559 | 1.00 | 20.95 | C |
| ATOM | 2345 | CG | ARG | B | 196 | −59.338 | 46.635 | 4.418 | 1.00 | 20.93 | C |
| ATOM | 2346 | CD | ARG | B | 196 | −58.095 | 46.921 | 5.240 | 1.00 | 20.80 | C |
| ATOM | 2347 | NE | ARG | B | 196 | −58.375 | 47.841 | 6.340 | 1.00 | 20.90 | N |
| ATOM | 2348 | CZ | ARG | B | 196 | −57.499 | 48.202 | 7.277 | 1.00 | 20.98 | C |
| ATOM | 2349 | NH1 | ARG | B | 196 | −56.258 | 47.723 | 7.279 | 1.00 | 21.03 | N |
| ATOM | 2350 | NH2 | ARG | B | 196 | −57.875 | 49.050 | 8.228 | 1.00 | 21.36 | N |
| ATOM | 2351 | C | ARG | B | 196 | −59.815 | 44.060 | 1.632 | 1.00 | 21.79 | C |
| ATOM | 2352 | O | ARG | B | 196 | −60.016 | 42.855 | 1.762 | 1.00 | 21.85 | O |
| ATOM | 2353 | N | VAL | B | 197 | −59.141 | 44.588 | 0.610 | 1.00 | 22.51 | N |
| ATOM | 2354 | CA | VAL | B | 197 | −58.396 | 43.770 | −0.356 | 1.00 | 23.28 | C |
| ATOM | 2355 | CB | VAL | B | 197 | −58.818 | 44.063 | −1.814 | 1.00 | 23.12 | C |
| ATOM | 2356 | CG1 | VAL | B | 197 | −57.888 | 43.383 | −2.818 | 1.00 | 23.19 | C |
| ATOM | 2357 | CG2 | VAL | B | 197 | −60.254 | 43.609 | −2.040 | 1.00 | 23.00 | C |
| ATOM | 2358 | C | VAL | B | 197 | −56.896 | 44.006 | −0.157 | 1.00 | 24.36 | C |
| ATOM | 2359 | O | VAL | B | 197 | −56.457 | 45.138 | 0.056 | 1.00 | 24.54 | O |
| ATOM | 2360 | N | GLU | B | 198 | −56.135 | 42.915 | −0.233 | 1.00 | 25.41 | N |
| ATOM | 2361 | CA | GLU | B | 198 | −54.685 | 42.906 | −0.069 | 1.00 | 26.25 | C |
| ATOM | 2362 | CB | GLU | B | 198 | −54.290 | 41.655 | 0.717 | 1.00 | 26.63 | C |
| ATOM | 2363 | CG | GLU | B | 198 | −52.829 | 41.566 | 1.136 | 1.00 | 27.07 | C |
| ATOM | 2364 | CO | GLU | B | 198 | −52.547 | 40.369 | 2.030 | 1.00 | 27.51 | C |
| ATOM | 2365 | OE1 | GLU | B | 198 | −53.431 | 39.493 | 2.176 | 1.00 | 27.92 | O |
| ATOM | 2366 | OE2 | GLU | B | 198 | −51.431 | 40.298 | 2.589 | 1.00 | 27.94 | O |
| ATOM | 2367 | C | GLU | B | 198 | −54.020 | 42.876 | −1.445 | 1.00 | 26.83 | C |
| ATOM | 2368 | O | GLU | B | 198 | −54.467 | 42.146 | −2.335 | 1.00 | 27.65 | O |
| ATOM | 2369 | N | GLY | B | 199 | −52.970 | 43.674 | −1.618 | 1.00 | 27.02 | N |
| ATOM | 2370 | CA | GLY | B | 199 | −52.118 | 43.606 | −2.808 | 1.00 | 27.18 | C |
| ATOM | 2371 | C | GLY | B | 199 | −52.771 | 44.017 | −4.114 | 1.00 | 26.98 | C |
| ATOM | 2372 | O | GLY | B | 199 | −52.565 | 43.372 | −5.142 | 1.00 | 27.26 | O |
| ATOM | 2373 | N | ASN | B | 200 | −53.560 | 45.087 | −4.071 | 1.00 | 26.61 | N |
| ATOM | 2374 | CA | ASN | B | 200 | −54.173 | 45.658 | −5.265 | 1.00 | 26.45 | C |
| ATOM | 2375 | CB | ASN | B | 200 | −55.579 | 45.084 | −5.466 | 1.00 | 26.67 | C |
| ATOM | 2376 | CG | ASN | B | 200 | −56.119 | 45.317 | −6.864 | 1.00 | 26.18 | C |
| ATOM | 2377 | OD1 | ASN | B | 200 | −55.688 | 46.221 | −7.584 | 1.00 | 27.02 | O |
| ATOM | 2378 | ND2 | ASN | B | 200 | −57.803 | 44.498 | −7.253 | 1.00 | 26.35 | N |
| ATOM | 2379 | C | ASN | B | 200 | −54.218 | 47.177 | −5.104 | 1.00 | 26.78 | C |
| ATOM | 2380 | O | ASN | B | 200 | −54.881 | 47.693 | −4.200 | 1.00 | 26.89 | O |
| ATOM | 2381 | N | LEU | B | 201 | −53.510 | 47.886 | −5.982 | 1.00 | 26.72 | N |
| ATOM | 2382 | CA | LEU | B | 201 | −53.363 | 49.344 | −5.864 | 1.00 | 26.62 | C |
| ATOM | 2383 | CB | LEU | B | 201 | −52.026 | 49.799 | −6.461 | 1.00 | 27.20 | C |
| ATOM | 2384 | CG | LEU | B | 201 | −51.267 | 50.832 | −5.628 | 1.00 | 27.44 | C |
| ATOM | 2385 | CD1 | LEU | B | 201 | −50.708 | 50.204 | −4.356 | 1.00 | 27.67 | C |
| ATOM | 2386 | CD2 | LEU | B | 201 | −50.151 | 51.456 | −6.451 | 1.00 | 27.61 | C |
| ATOM | 2387 | C | LEU | B | 201 | −54.530 | 50.099 | −6.506 | 1.00 | 25.97 | C |
| ATOM | 2388 | O | LEU | B | 201 | −54.788 | 51.528 | −6.165 | 1.00 | 27.14 | O |
| ATOM | 2389 | N | ARG | B | 202 | −55.240 | 49.433 | −7.414 | 1.00 | 24.95 | N |
| ATOM | 2390 | CA | ARG | B | 202 | −56.464 | 49.965 | −8.013 | 1.00 | 24.29 | C |
| ATOM | 2391 | CB | ARG | B | 202 | −56.659 | 49.362 | −9.417 | 1.00 | 24.68 | C |
| ATOM | 2392 | CG | ARG | B | 202 | −55.506 | 49.638 | −10.384 | 1.00 | 25.08 | C |
| ATOM | 2393 | CD | ARG | B | 202 | −55.499 | 48.685 | −11.576 | 1.00 | 25.59 | C |
| ATOM | 2394 | NE | ARG | B | 202 | −56.587 | 48.948 | −12.519 | 1.00 | 26.03 | N |
| ATOM | 2395 | CZ | ARG | B | 202 | −56.997 | 48.118 | −13.485 | 1.00 | 26.21 | C |
| ATOM | 2396 | NH1 | ARG | B | 202 | −56.427 | 46.924 | −13.676 | 1.00 | 26.40 | N |
| ATOM | 2397 | NH2 | ARG | B | 202 | −58.012 | 48.485 | −14.265 | 1.00 | 26.25 | N |
| ATOM | 2398 | C | ARG | B | 202 | −57.713 | 49.725 | −7.141 | 1.00 | 23.26 | C |
| ATOM | 2399 | O | ARG | B | 202 | −58.832 | 49.973 | −7.597 | 1.00 | 23.14 | O |
| ATOM | 2400 | N | VAL | B | 203 | −57.528 | 49.233 | −5.909 | 1.00 | 22.59 | N |
| ATOM | 2401 | CA | VAL | B | 203 | −58.627 | 49.075 | −4.936 | 1.00 | 22.04 | C |
| ATOM | 2402 | CB | VAL | B | 203 | −58.134 | 48.479 | −3.581 | 1.00 | 21.98 | C |
| ATOM | 2403 | CG1 | VAL | B | 203 | −57.181 | 49.418 | −2.846 | 1.00 | 21.97 | C |
| ATOM | 2404 | CG2 | VAL | B | 203 | −59.310 | 48.106 | −2.684 | 1.00 | 21.96 | C |
| ATOM | 2405 | C | VAL | B | 203 | −59.390 | 50.381 | −4.690 | 1.00 | 21.58 | C |
| ATOM | 2406 | O | VAL | B | 203 | −58.788 | 51.448 | −4.540 | 1.00 | 21.51 | O |
| ATOM | 2407 | N | GLU | B | 204 | −60.717 | 50.274 | −4.661 | 1.00 | 21.10 | N |
| ATOM | 2408 | CA | GLU | B | 204 | −61.606 | 51.405 | −4.417 | 1.00 | 21.01 | C |
| ATOM | 2409 | CB | GLU | B | 204 | −62.340 | 51.767 | −5.708 | 1.00 | 21.07 | C |
| ATOM | 2410 | CG | GLU | B | 204 | −63.401 | 52.851 | −5.582 | 1.00 | 21.45 | C |
| ATOM | 2411 | CD | GLU | B | 204 | −64.197 | 53.036 | −6.860 | 1.00 | 21.74 | C |
| ATOM | 2412 | OE1 | GLU | B | 204 | −63.600 | 52.996 | −7.956 | 1.00 | 21.73 | O |
| ATOM | 2413 | OE2 | GLU | B | 204 | −65.425 | 53.226 | −6.767 | 1.00 | 22.20 | O |
| ATOM | 2414 | C | GLU | B | 204 | −62.598 | 51.038 | −3.307 | 1.00 | 20.64 | C |
| ATOM | 2415 | O | GLU | B | 204 | −63.122 | 49.926 | −3.285 | 1.00 | 20.15 | O |
| ATOM | 2416 | N | TYR | B | 205 | −62.857 | 51.990 | −2.410 | 1.00 | 20.43 | N |
| ATOM | 2417 | CA | TYR | B | 205 | −63.724 | 51.782 | −1.248 | 1.00 | 20.18 | C |
| ATOM | 2418 | CB | TYR | B | 205 | −62.926 | 51.996 | 0.029 | 1.00 | 20.09 | C |
| ATOM | 2419 | CG | TYR | B | 205 | −61.932 | 50.904 | 0.304 | 1.00 | 19.82 | C |
| ATOM | 2420 | C | TYR | B | 205 | −62.322 | 49.733 | 0.953 | 1.00 | 19.70 | C |
| ATOM | 2421 | OE1 | TYR | B | 205 | −61.411 | 48.723 | 1.221 | 1.00 | 19.81 | C |
| ATOM | 2422 | CZ | TYR | B | 205 | −60.087 | 48.872 | 0.836 | 1.00 | 19.73 | C |
| ATOM | 2423 | OH | TYR | B | 205 | −59.191 | 47.863 | 1.107 | 1.00 | 19.41 | O |
| ATOM | 2424 | CE2 | TYR | B | 205 | −59.673 | 50.031 | 0.192 | 1.00 | 19.74 | C |
| ATOM | 2425 | CD2 | TYR | B | 205 | −60.593 | 51.039 | −0.070 | 1.00 | 19.84 | C |
| ATOM | 2426 | C | TYR | B | 205 | −64.896 | 52.750 | −1.274 | 1.00 | 20.37 | C |
| ATOM | 2427 | O | TYR | B | 205 | −64.697 | 53.956 | −1.462 | 1.00 | 20.69 | O |
| ATOM | 2428 | N | LEU | B | 206 | −66.107 | 52.221 | −1.078 | 1.00 | 20.26 | N |
| ATOM | 2429 | CA | LEU | B | 206 | −67.340 | 53.017 | −1.130 | 1.00 | 20.60 | C |
| ATOM | 2430 | CB | LEU | B | 206 | −68.275 | 52.478 | −2.223 | 1.00 | 20.20 | C |
| ATOM | 2431 | CG | LEU | B | 206 | −69.688 | 53.079 | −2.305 | 1.00 | 20.35 | C |
| ATOM | 2432 | CD1 | LEU | B | 206 | −69.635 | 54.585 | −2.524 | 1.00 | 20.57 | C |
| ATOM | 2433 | CD2 | LEU | B | 206 | −70.510 | 52.411 | −3.394 | 1.00 | 20.26 | C |
| ATOM | 2434 | C | LEU | B | 206 | −68.080 | 53.013 | 0.209 | 1.00 | 20.95 | O |
| ATOM | 2435 | O | LEU | B | 206 | −68.334 | 51.949 | 0.770 | 1.00 | 20.42 | N |
| ATOM | 2436 | N | LEU | B | 207 | −68.402 | 54.210 | 0.705 | 1.00 | 21.64 | C |
| ATOM | 2437 | CA | ASP | B | 207 | −69.483 | 54.420 | 1.669 | 1.00 | 22.34 | C |
| ATOM | 2438 | CB | ASP | B | 207 | −69.102 | 55.474 | 2.716 | 1.00 | 22.62 | C |
| ATOM | 2439 | CG | ASP | B | 207 | −67.933 | 55.057 | 3.582 | 1.00 | 22.53 | O |
| ATOM | 2440 | OD1 | ASP | B | 207 | −67.427 | 53.927 | 3.422 | 1.00 | 22.27 | O |
| ATOM | 2441 | OD2 | ASP | B | 207 | −67.520 | 55.872 | 4.431 | 1.00 | 22.64 | C |
| ATOM | 2442 | C | ASP | B | 207 | −70.670 | 54.938 | 0.865 | 1.00 | 23.41 | O |
| ATOM | 2443 | O | ASP | B | 207 | −70.631 | 56.068 | 0.366 | 1.00 | 23.55 | N |
| ATOM | 2444 | N | ASP | B | 208 | −71.716 | 54.125 | 0.726 | 1.00 | 24.10 | C |
| ATOM | 2445 | CA | ASP | B | 208 | −72.864 | 54.490 | −0.109 | 1.00 | 25.11 | C |
| ATOM | 2446 | CB | ASP | B | 208 | −73.787 | 53.286 | −0.293 | 1.00 | 25.35 | C |
| ATOM | 2447 | CG | ASP | B | 208 | −74.877 | 53.528 | −1.323 | 1.00 | 25.47 | C |
| ATOM | 2448 | OD1 | ASP | B | 208 | −75.769 | 54.360 | −1.068 | 1.00 | 25.59 | O |
| ATOM | 2449 | OD2 | ASP | B | 208 | −74.856 | 52.865 | −2.383 | 1.00 | 26.14 | O |
| ATOM | 2450 | C | ASP | B | 208 | −73.616 | 55.668 | 0.518 | 1.00 | 26.07 | C |
| ATOM | 2451 | O | ASP | B | 208 | −73.849 | 55.682 | 1.729 | 1.00 | 25.45 | O |
| ATOM | 2452 | N | ARG | B | 209 | −73.985 | 56.646 | −0.311 | 1.00 | 27.25 | N |
| ATOM | 2453 | CA | ARG | B | 209 | −74.576 | 57.898 | 0.184 | 1.00 | 28.79 | C |
| ATOM | 2454 | CB | ARG | B | 209 | −74.571 | 58.988 | −0.910 | 1.00 | 30.34 | C |
| ATOM | 2455 | CO | ARG | B | 209 | −15.674 | 58.880 | −1.959 | 1.00 | 31.63 | C |
| ATOM | 2456 | CD | ARG | B | 209 | −75.508 | 59.896 | −3.079 | 1.00 | 33.19 | C |
| ATOM | 2457 | NE | ARG | B | 209 | −76.594 | 59.802 | −4.059 | 1.00 | 34.35 | N |
| ATOM | 2458 | CZ | ARG | B | 209 | −76.704 | 58.867 | −5.009 | 1.00 | 35.03 | C |
| ATOM | 2459 | NE1 | ARG | B | 209 | −75.791 | 57.903 | −5.147 | 1.00 | 35.79 | N |
| ATOM | 2460 | NE2 | ARG | B | 209 | −77.747 | 58.895 | −5.839 | 1.00 | 35.37 | N |
| ATOM | 2461 | C | ARG | B | 209 | −75.987 | 57.722 | 0.774 | 1.00 | 28.59 | C |
| ATOM | 2462 | O | ARG | B | 209 | −76.347 | 58.419 | 1.723 | 1.00 | 28.90 | O |
| ATOM | 2463 | N | ASN | B | 210 | −76.767 | 56.796 | 0.215 | 1.00 | 28.99 | N |
| ATOM | 2464 | CA | ASN | B | 210 | −78.166 | 56.590 | 0.615 | 1.00 | 28.48 | C |
| ATOM | 2465 | CB | ASN | B | 210 | −79.044 | 56.415 | −0.626 | 1.00 | 28.57 | C |
| ATOM | 2466 | CG | ASN | B | 210 | −78.949 | 57.594 | −1.578 | 1.00 | 28.73 | C |
| ATOM | 2467 | OG1 | ASN | B | 210 | −78.597 | 57.435 | −2.746 | 1.00 | 28.72 | O |
| ATOM | 2468 | ND2 | ASN | B | 210 | −79.245 | 58.790 | −1.076 | 1.00 | 28.26 | N |
| ATOM | 2469 | C | ASN | B | 210 | −78.359 | 55.412 | 1.577 | 1.00 | 28.23 | C |
| ATOM | 2470 | O | ASN | B | 210 | −79.085 | 55.541 | 2.565 | 1.00 | 28.75 | C |
| ATOM | 2471 | N | THR | B | 211 | −77.718 | 54.275 | 1.297 | 1.00 | 27.62 | N |
| ATOM | 2472 | CA | THR | B | 211 | −77.826 | 53.089 | 2.164 | 1.00 | 27.11 | C |
| ATOM | 2473 | CB | THR | B | 211 | −77.588 | 51.769 | 1.388 | 1.00 | 27.28 | C |
| ATOM | 2474 | OG1 | THR | B | 211 | −76.220 | 51.683 | 0.969 | 1.00 | 26.86 | O |
| ATOM | 2475 | CG2 | THR | B | 211 | −78.499 | 51.670 | 0.173 | 1.00 | 27.44 | C |
| ATOM | 2476 | C | THR | B | 211 | −76.876 | 53.101 | 3.372 | 1.00 | 26.30 | C |
| ATOM | 2477 | O | THR | B | 211 | −77.131 | 52.396 | 4.346 | 1.00 | 26.02 | O |
| ATOM | 2478 | N | PHE | B | 212 | −15.785 | 53.874 | 3.289 | 1.00 | 25.59 | N |
| ATOM | 2479 | CA | PHE | B | 212 | −74.701 | 53.911 | 4.302 | 1.00 | 25.18 | C |
| ATOM | 2480 | CB | PHE | B | 212 | −75.221 | 54.335 | 5.691 | 1.00 | 25.84 | C |
| ATOM | 2481 | CG | PHE | B | 212 | −75.744 | 55.745 | 5.740 | 1.00 | 26.52 | C |
| ATOM | 2482 | CD1 | PHE | B | 212 | −77.028 | 56.046 | 5.289 | 1.00 | 27.03 | C |
| ATOM | 2483 | CE1 | PHE | B | 212 | −77.512 | 57.349 | 5.338 | 1.00 | 27.44 | C |
| ATOM | 2484 | CZ | PHE | B | 212 | −76.714 | 58.367 | 5.845 | 1.00 | 27.55 | C |
| ATOM | 2485 | CE2 | PHE | B | 212 | −75.436 | 58.080 | 6.306 | 1.00 | 27.25 | C |
| ATOM | 2486 | CD2 | PHE | B | 212 | −74.956 | 56.778 | 6.252 | 1.00 | 27.01 | C |
| ATOM | 2487 | C | PHE | B | 212 | −73.856 | 52.624 | 4.397 | 1.00 | 24.04 | C |
| ATOM | 2488 | O | PHE | B | 212 | −73.007 | 52.501 | 5.287 | 1.00 | 24.00 | O |
| ATOM | 2489 | N | ARG | B | 213 | −74.038 | 51.702 | 3.452 | 1.00 | 22.45 | N |
| ATOM | 2490 | CA | ARG | B | 213 | −73.358 | 50.408 | 3.484 | 1.00 | 21.60 | C |
| ATOM | 2491 | CB | ARG | B | 213 | −74.175 | 49.355 | 2.738 | 1.00 | 21.66 | C |
| ATOM | 2492 | CG | ARG | B | 213 | −74.490 | 49.013 | 3.413 | 1.00 | 21.77 | C |
| ATOM | 2493 | CD | ARG | B | 213 | −76.002 | 47.667 | 2.941 | 1.00 | 21.74 | C |
| ATOM | 2494 | NE | ARG | B | 213 | −77.336 | 47.367 | 3.463 | 1.00 | 21.88 | N |
| ATOM | 2495 | CZ | ARG | B | 213 | −77.614 | 49.688 | 4.581 | 1.00 | 21.60 | C |
| ATOM | 2496 | NH1 | ARG | B | 213 | −76.659 | 46.412 | 5.387 | 1.00 | 21.32 | N |
| ATOM | 2497 | NH2 | ARG | B | 213 | −78.887 | 46.484 | 4.905 | 1.00 | 21.68 | N |
| ATOM | 2498 | C | ARG | B | 213 | −71.968 | 50.509 | 2.868 | 1.00 | 20.52 | C |
| ATOM | 2499 | O | ARG | B | 213 | −71.752 | 51.265 | 1.913 | 1.00 | 20.30 | O |
| ATOM | 2500 | N | HIS | B | 214 | −71.036 | 49.732 | 3.417 | 1.00 | 19.37 | N |
| ATOM | 2501 | CA | HIS | B | 214 | −69.642 | 49.740 | 2.978 | 1.00 | 18.39 | C |
| ATOM | 2502 | CB | HIS | B | 214 | −68.714 | 49.541 | 4.168 | 1.00 | 17.99 | C |
| ATOM | 2503 | CG | HIS | B | 214 | −68.872 | 50.580 | 5.230 | 1.00 | 17.93 | C |
| ATOM | 2504 | ND1 | HIS | B | 214 | −68.501 | 50.364 | 6.539 | 1.00 | 17.64 | N |
| ATOM | 2505 | CE1 | HIS | B | 214 | −68.764 | 51.447 | 7.249 | 1.00 | 17.84 | C |
| ATOM | 2506 | NE1 | HIS | B | 214 | −69.301 | 52.351 | 6.449 | 1.00 | 17.83 | N |
| ATOM | 2507 | CD2 | HIS | B | 214 | −69.382 | 51.834 | 5.181 | 1.00 | 17.53 | C |
| ATOM | 2508 | C | HIS | B | 214 | −69.734 | 48.649 | 1.949 | 1.00 | 18.15 | C |
| ATOM | 2509 | O | HIS | B | 214 | −69.958 | 47.568 | 2.016 | 1.00 | 18.18 | O |
| ATOM | 2510 | N | SER | B | 215 | −68.489 | 48.938 | 1.000 | 1.00 | 17.57 | N |
| ATOM | 2511 | CA | SER | B | 215 | −68.032 | 47.930 | 0.046 | 1.00 | 17.49 | C |
| ATOM | 2512 | CB | SER | B | 215 | −69.040 | 47.753 | −1.095 | 1.00 | 17.42 | C |
| ATOM | 2513 | OG | SER | B | 215 | −69.136 | 48.922 | −1.886 | 1.00 | 17.46 | O |
| ATOM | 2514 | C | SER | B | 215 | −66.662 | 48.271 | −0.513 | 1.00 | 17.35 | C |
| ATOM | 2515 | O | SER | B | 215 | −66.228 | 49.428 | −0.482 | 1.00 | 16.78 | O |
| ATOM | 2516 | N | VAL | B | 216 | −65.992 | 47.242 | −1.019 | 1.00 | 17.57 | N |
| ATOM | 2517 | CA | VAL | B | 216 | −64.655 | 47.369 | −1.601 | 1.00 | 18.20 | C |
| ATOM | 2518 | CB | VAL | B | 216 | −63.550 | 46.708 | −0.722 | 1.00 | 18.33 | C |
| ATOM | 2519 | CG1 | VAL | B | 216 | −63.891 | 45.268 | −0.332 | 1.00 | 18.38 | C |
| ATOM | 2520 | CG2 | VAL | B | 216 | −62.196 | 46.763 | −1.421 | 1.00 | 18.49 | C |
| ATOM | 2521 | C | VAL | B | 216 | −64.716 | 46.765 | −2.995 | 1.00 | 18.49 | C |
| ATOM | 2522 | O | VAL | B | 216 | −65.264 | 45.679 | −3.174 | 1.00 | 18.75 | O |
| ATOM | 2523 | N | VAL | B | 217 | −64.165 | 47.480 | −3.973 | 1.00 | 18.94 | N |
| ATOM | 2524 | CA | VAL | B | 217 | −64.287 | 47.099 | −5.377 | 1.00 | 19.22 | C |
| ATOM | 2525 | CB | VAL | B | 217 | −65.415 | 47.894 | −6.089 | 1.00 | 19.13 | C |
| ATOM | 2526 | CG1 | VAL | B | 217 | −65.151 | 49.394 | −6.084 | 1.00 | 19.16 | C |
| ATOM | 2527 | CG2 | VAL | B | 217 | −65.628 | 47.384 | −7.510 | 1.00 | 18.92 | C |
| ATOM | 2528 | C | VAL | B | 217 | −62.943 | 47.206 | −6.106 | 1.00 | 19.80 | C |
| ATOM | 2529 | O | VAL | B | 217 | −62.240 | 48.216 | −5.991 | 1.00 | 19.61 | O |
| ATOM | 2530 | N | VAL | B | 218 | −62.593 | 46.140 | −6.827 | 1.00 | 20.33 | N |
| ATOM | 2531 | CA | VAL | B | 218 | −61.386 | 46.094 | −7.654 | 1.00 | 21.08 | C |
| ATOM | 2532 | CB | VAL | B | 218 | −60.381 | 44.992 | −7.219 | 1.00 | 20.99 | C |
| ATOM | 2533 | CG1 | VAL | B | 218 | −59.877 | 45.253 | −5.809 | 1.00 | 20.74 | C |
| ATOM | 2534 | CG2 | VAL | B | 218 | −60.965 | 43.581 | −7.344 | 1.00 | 20.95 | C |
| ATOM | 2535 | C | VAL | B | 218 | −61.766 | 45.847 | −9.110 | 1.00 | 21.97 | C |
| ATOM | 2536 | O | VAL | B | 218 | −62.865 | 45.355 | −9.389 | 1.00 | 21.98 | O |
| ATOM | 2537 | N | PRO | B | 219 | −60.865 | 46.199 | −10.046 | 1.00 | 23.29 | N |
| ATOM | 2538 | CA | PRO | B | 219 | −61.025 | 45.755 | −11.427 | 1.00 | 23.76 | C |
| ATOM | 2539 | CB | PRO | B | 219 | −59.875 | 46.461 | −12.151 | 1.00 | 23.79 | C |
| ATOM | 2540 | CG | PRO | B | 219 | −59.688 | 47.713 | −11.368 | 1.00 | 23.54 | C |
| ATOM | 2541 | CD | PRO | B | 219 | −59.888 | 47.300 | −9.946 | 1.00 | 23.43 | C |
| ATOM | 2542 | C | PRO | B | 219 | −60.912 | 44.236 | −11.583 | 1.00 | 24.51 | C |
| ATOM | 2543 | O | PRO | B | 219 | −59.980 | 43.618 | −11.055 | 1.00 | 24.19 | O |
| ATOM | 2544 | N | TYR | B | 220 | −61.877 | 43.652 | −12.289 | 1.00 | 25.61 | N |
| ATOM | 2545 | CA | TYR | B | 220 | −61.800 | 42.264 | −12.710 | 1.00 | 26.56 | C |
| ATOM | 2546 | CB | TYR | B | 220 | −63.110 | 41.814 | −13.368 | 1.00 | 26.49 | C |
| ATOM | 2547 | CG | TYR | B | 220 | −63.010 | 40.446 | −13.992 | 1.00 | 26.34 | C |
| ATOM | 2548 | CD1 | TYR | B | 220 | −63.198 | 39.296 | −13.230 | 1.00 | 26.15 | C |
| ATOM | 2549 | CE1 | TYR | B | 220 | −63.092 | 38.037 | −13.799 | 1.00 | 26.14 | C |
| ATOM | 2550 | CZ | TYR | B | 220 | −62.783 | 37.919 | −15.146 | 1.00 | 26.28 | C |
| ATOM | 2551 | OH | TYR | B | 220 | −62.674 | 36.679 | −15.719 | 1.00 | 26.21 | O |
| ATOM | 2552 | CE2 | TYR | B | 220 | −62.579 | 39.045 | −15.921 | 1.00 | 26.46 | C |
| ATOM | 2553 | CD2 | TYR | B | 220 | −62.695 | 40.298 | −15.346 | 1.00 | 26.44 | C |
| ATOM | 2554 | C | TYR | B | 220 | −60.644 | 42.097 | −13.695 | 1.00 | 27.84 | C |
| ATOM | 2555 | O | GLU | B | 220 | −60.549 | 42.837 | −14.679 | 1.00 | 28.12 | O |
| ATOM | 2556 | N | GLU | B | 221 | −59.769 | 41.135 | −13.411 | 1.00 | 29.43 | N |
| ATOM | 2557 | CA | GLU | B | 221 | −58.716 | 40.713 | −14.329 | 1.00 | 30.40 | C |
| ATOM | 2558 | CB | GLU | B | 221 | −57.340 | 40.815 | −13.663 | 1.00 | 31.14 | C |
| ATOM | 2559 | CG | GLU | B | 221 | −56.848 | 42.236 | −13.406 | 1.00 | 31.76 | C |
| ATOM | 2560 | CD | GLU | B | 221 | −56.399 | 42.981 | −14.660 | 1.00 | 32.11 | C |
| ATOM | 2561 | OE1 | GLU | B | 221 | −56.396 | 42.401 | −15.770 | 1.00 | 32.82 | O |
| ATOM | 2562 | OE2 | GLU | B | 221 | −56.039 | 44.168 | −14.529 | 1.00 | 31.94 | O |
| ATOM | 2563 | C | GLU | B | 221 | −58.995 | 39.259 | −14.698 | 1.00 | 30.43 | C |
| ATOM | 2564 | O | GLU | B | 221 | −59.401 | 38.477 | −13.835 | 1.00 | 30.01 | O |
| ATOM | 2565 | N | PRO | B | 220 | −58.783 | 38.883 | −15.975 | 1.00 | 31.18 | N |
| ATOM | 2566 | CA | PRO | B | 220 | −58.946 | 37.478 | −16.346 | 1.00 | 31.47 | C |
| ATOM | 2567 | CB | PRO | B | 220 | −58.893 | 37.516 | −17.874 | 1.00 | 31.54 | C |
| ATOM | 2568 | CG | PRO | B | 220 | −58.029 | 38.684 | −18.183 | 1.00 | 31.65 | C |
| ATOM | 2569 | CD | PRO | B | 222 | −58.323 | 39.698 | −17.116 | 1.00 | 31.51 | C |
| ATOM | 2570 | C | PRO | B | 222 | −57.808 | 36.633 | −15.767 | 1.00 | 32.03 | C |
| ATOM | 2571 | O | PRO | B | 222 | −56.803 | 37.197 | −15.325 | 1.00 | 31.22 | O |
| ATOM | 2572 | N | PRO | B | 223 | −57.957 | 35.293 | −15.160 | 1.00 | 33.21 | N |
| ATOM | 2573 | CA | PRO | B | 223 | −56.937 | 34.460 | −15.110 | 1.00 | 34.41 | C |
| ATOM | 2574 | CB | PRO | B | 223 | −57.445 | 33.025 | −15.331 | 1.00 | 33.93 | C |
| ATOM | 2575 | CG | PRO | B | 223 | −58.494 | 33.117 | −16.385 | 1.00 | 33.68 | C |
| ATOM | 2576 | CD | PRO | B | 223 | −59.079 | 34.488 | −16.274 | 1.00 | 33.27 | C |
| ATOM | 2577 | C | PRO | B | 223 | −55.525 | 34.626 | −15.689 | 1.00 | 35.94 | C |
| ATOM | 2578 | O | PRO | B | 223 | −55.368 | 34.943 | −16.870 | 1.00 | 36.28 | O |
| ATOM | 2579 | N | GLU | B | 224 | −54.522 | 34.430 | −14.837 | 1.00 | 37.98 | N |
| ATOM | 2580 | CA | GLU | B | 224 | −53.129 | 34.371 | −15.271 | 1.00 | 39.93 | C |
| ATOM | 2581 | CB | GLU | B | 224 | −52.175 | 34.244 | −14.073 | 1.00 | 40.50 | C |
| ATOM | 2582 | CG | GLU | B | 224 | −52.266 | 35.342 | −13.016 | 1.00 | 40.89 | C |
| ATOM | 2583 | CD | GLU | B | 224 | −51.173 | 36.384 | −13.141 | 1.00 | 41.15 | C |
| ATOM | 2584 | OE1 | GLU | B | 224 | −50.260 | 36.387 | −12.288 | 1.00 | 41.65 | O |
| ATOM | 2585 | OE2 | GLU | B | 224 | −51.226 | 37.192 | −14.092 | 1.00 | 41.48 | O |
| ATOM | 2586 | C | GLU | B | 224 | −53.030 | 33.107 | −16.113 | 1.00 | 41.31 | C |
| ATOM | 2587 | O | GLU | B | 224 | −53.398 | 32.033 | −15.629 | 1.00 | 41.87 | O |
| ATOM | 2588 | N | VAL | B | 225 | −52.561 | 33.218 | −17.359 | 1.00 | 42.64 | N |
| ATOM | 2589 | CA | VAL | B | 225 | −52.472 | 32.035 | −18.226 | 1.00 | 42.95 | C |
| ATOM | 2590 | CB | VAL | B | 225 | −52.045 | 32.344 | −19.682 | 1.00 | 43.52 | C |
| ATOM | 2591 | CG1 | VAL | B | 225 | −51.992 | 31.056 | −20.505 | 1.00 | 43.67 | C |
| ATOM | 2592 | CG2 | VAL | B | 225 | −53.017 | 33.328 | −20.325 | 1.00 | 43.44 | C |
| ATOM | 2593 | C | VAL | B | 225 | −51.516 | 31.064 | −17.532 | 1.00 | 43.29 | C |
| ATOM | 2594 | O | VAL | B | 225 | −50.380 | 31.413 | −17.194 | 1.00 | 43.35 | O |
| ATOM | 2595 | N | GLY | B | 226 | −52.013 | 29.849 | −17.330 | 1.00 | 42.79 | N |
| ATOM | 2596 | CA | GLY | B | 226 | −51.604 | 28.995 | −16.226 | 1.00 | 42.74 | C |
| ATOM | 2597 | C | GLY | B | 226 | −52.896 | 28.679 | −15.495 | 1.00 | 42.36 | C |
| ATOM | 2598 | O | GLY | B | 226 | −53.695 | 27.890 | −15.984 | 1.00 | 43.18 | O |
| ATOM | 2599 | N | SER | B | 227 | −53.136 | 29.341 | −14.363 | 1.00 | 41.55 | N |
| ATOM | 2600 | CA | SER | B | 227 | −54.376 | 29.156 | −13.586 | 1.00 | 40.50 | C |
| ATOM | 2601 | CB | SER | B | 227 | −54.368 | 30.077 | −12.359 | 1.00 | 40.25 | C |
| ATOM | 2602 | OG | SER | B | 227 | −55.496 | 29.849 | −11.531 | 1.00 | 40.45 | O |
| ATOM | 2603 | C | SER | B | 227 | −55.645 | 29.411 | −14.418 | 1.00 | 39.45 | C |
| ATOM | 2604 | O | SER | B | 227 | −55.601 | 30.133 | −15.414 | 1.00 | 39.24 | O |
| ATOM | 2605 | N | ASP | B | 228 | −56.760 | 28.798 | −14.013 | 1.00 | 39.58 | N |
| ATOM | 2606 | CA | ASP | B | 228 | −58.068 | 29.014 | −14.662 | 1.00 | 39.77 | C |
| ATOM | 2607 | CB | ASP | B | 228 | −58.633 | 27.681 | −15.188 | 1.00 | 41.64 | C |
| ATOM | 2608 | CG | ASP | B | 228 | −58.082 | 27.300 | −16.562 | 1.00 | 42.69 | C |
| ATOM | 2609 | OD1 | ASP | B | 228 | −57.112 | 27.931 | −17.046 | 1.00 | 43.68 | O |
| ATOM | 2610 | OD2 | ASP | B | 228 | −58.629 | 26.345 | −17.162 | 1.00 | 44.43 | O |
| ATOM | 2611 | C | ASP | B | 228 | −59.081 | 29.732 | −13.753 | 1.00 | 38.09 | C |
| ATOM | 2612 | O | ASP | B | 228 | −60.297 | 29.621 | −13.946 | 1.00 | 37.98 | O |
| ATOM | 2613 | N | CYS | B | 229 | −58.571 | 30.493 | −12.786 | 1.00 | 35.68 | N |
| ATOM | 2614 | CA | CYS | B | 229 | −59.403 | 31.493 | −11.975 | 1.00 | 34.11 | C |
| ATOM | 2615 | CB | CYS | B | 229 | −59.915 | 30.640 | −10.741 | 1.00 | 33.56 | C |
| ATOM | 2616 | SG | CYS | B | 229 | −58.612 | 29.955 | −9.699 | 1.00 | 33.88 | S |
| ATOM | 2617 | C | CYS | B | 229 | −58.640 | 32.627 | −11.555 | 1.00 | 32.49 | C |
| ATOM | 2618 | O | CYS | B | 229 | −57.405 | 32.661 | −11.577 | 1.00 | 32.01 | O |
| ATOM | 2619 | N | THR | B | 230 | −59.402 | 33.650 | −11.182 | 1.00 | 31.46 | N |
| ATOM | 2620 | CA | THR | B | 230 | −58.868 | 34.917 | −10.696 | 1.00 | 30.62 | C |
| ATOM | 2621 | CB | THR | B | 230 | −59.618 | 36.096 | −11.338 | 1.00 | 30.34 | C |
| ATOM | 2622 | OG1 | THR | B | 230 | −59.402 | 36.068 | −12.753 | 1.00 | 30.24 | O |
| ATOM | 2623 | CG2 | THR | B | 230 | −59.146 | 37.438 | −10.769 | 1.00 | 30.25 | C |
| ATOM | 2624 | C | THR | B | 230 | −59.048 | 34.957 | −9.190 | 1.00 | 29.53 | C |
| ATOM | 2625 | O | THR | B | 230 | −60.174 | 34.869 | −8.708 | 1.00 | 29.41 | O |
| ATOM | 2626 | N | THR | B | 231 | −57.949 | 35.097 | −8.454 | 1.00 | 28.69 | N |
| ATOM | 2627 | CA | THR | B | 231 | −57.992 | 35.121 | −6.995 | 1.00 | 28.06 | C |
| ATOM | 2628 | CB | THR | B | 231 | −56.834 | 34.299 | −6.389 | 1.00 | 28.55 | C |
| ATOM | 2629 | OG1 | THR | B | 231 | −56.717 | 33.048 | −7.078 | 1.00 | 28.25 | O |
| ATOM | 2630 | CG2 | THR | B | 231 | −57.070 | 34.031 | −4.902 | 1.00 | 28.74 | C |
| ATOM | 2631 | C | THR | B | 231 | −57.916 | 36.564 | −6.490 | 1.00 | 27.52 | C |
| ATOM | 2632 | O | THR | B | 231 | −57.023 | 37.318 | −6.890 | 1.00 | 27.03 | O |
| ATOM | 2633 | N | ILE | B | 232 | −58.870 | 36.944 | −5.639 | 1.00 | 26.72 | N |
| ATOM | 2634 | CA | ILE | B | 232 | −58.773 | 38.169 | −4.840 | 1.00 | 26.34 | C |
| ATOM | 2635 | CB | ILE | B | 232 | −60.101 | 38.958 | −4.819 | 1.00 | 26.28 | C |
| ATOM | 2636 | CG1 | ILE | B | 232 | −60.433 | 39.470 | −6.226 | 1.00 | 26.19 | C |
| ATOM | 2637 | CD1 | ILE | B | 232 | −61.887 | 39.841 | −6.416 | 1.00 | 26.04 | C |
| ATOM | 2638 | CG2 | ILE | B | 232 | −60.024 | 40.137 | −3.845 | 1.00 | 26.56 | C |
| ATOM | 2639 | C | ILE | B | 232 | −58.377 | 37.769 | −3.421 | 1.00 | 25.63 | C |
| ATOM | 2640 | O | ILE | B | 232 | −58.925 | 36.813 | −2.864 | 1.00 | 25.36 | O |
| ATOM | 2641 | N | HIS | B | 233 | −57.425 | 38.506 | −2.851 | 1.00 | 25.03 | N |
| ATOM | 2642 | CA | HIS | B | 233 | −57.002 | 38.319 | −2.466 | 1.00 | 24.75 | C |
| ATOM | 2643 | CE | HIS | B | 233 | −55.489 | 38.523 | −1.320 | 1.00 | 24.86 | C |
| ATOM | 2644 | CG | HIS | B | 233 | −54.666 | 37.374 | −1.813 | 1.00 | 25.31 | C |
| ATOM | 2645 | ND1 | HIS | B | 233 | −53.356 | 37.520 | −2.213 | 1.00 | 25.44 | N |
| ATOM | 2646 | CE1 | HIS | B | 233 | −52.880 | 36.347 | −2.593 | 1.00 | 25.37 | C |
| ATOM | 2647 | NE1 | HIS | B | 233 | −53.836 | 35.446 | −2.458 | 1.00 | 25.01 | N |
| ATOM | 2648 | CD2 | HIS | B | 233 | −54.963 | 36.061 | −1.970 | 1.00 | 25.23 | C |
| ATOM | 2649 | C | HIS | B | 233 | −57.743 | 39.305 | −0.573 | 1.00 | 24.45 | C |
| ATOM | 2650 | O | HIS | B | 233 | −57.345 | 40.466 | −0.458 | 1.00 | 24.85 | O |
| ATOM | 2651 | N | TYR | B | 234 | −58.824 | 38.847 | 0.051 | 1.00 | 23.65 | N |
| ATOM | 2652 | CA | TYR | B | 234 | −59.551 | 39.674 | 1.013 | 1.00 | 23.49 | C |
| ATOM | 2653 | CB | TYR | B | 234 | −61.027 | 39.271 | 1.088 | 1.00 | 23.06 | C |
| ATOM | 2654 | CG | TYR | B | 234 | −61.800 | 39.610 | −0.166 | 1.00 | 22.63 | C |
| ATOM | 2655 | COI | TYR | B | 234 | −62.208 | 40.920 | −0.424 | 1.00 | 22.36 | C |
| ATOM | 2656 | CE1 | TYR | B | 234 | −62.914 | 41.238 | −1.572 | 1.00 | 22.33 | C |
| ATOM | 2657 | CZ | TYR | B | 234 | −63.217 | 40.242 | −2.486 | 1.00 | 22.49 | C |
| ATOM | 2658 | OH | TYR | B | 234 | −63.912 | 40.551 | −3.633 | 1.00 | 22.68 | O |
| ATOM | 2659 | CE2 | TYR | B | 234 | −62.823 | 38.936 | −2.253 | 1.00 | 22.49 | C |
| ATOM | 2660 | CD2 | TYR | B | 234 | −62.117 | 38.629 | −1.100 | 1.00 | 22.33 | C |
| ATOM | 2661 | C | TYR | B | 234 | −58.901 | 39.613 | 2.396 | 1.00 | 23.65 | C |
| ATOM | 2662 | O | TYR | B | 234 | −58.211 | 38.649 | 2.729 | 1.00 | 23.47 | O |
| ATOM | 2663 | N | ASN | B | 235 | −59.103 | 40.674 | 3.173 | 1.00 | 24.01 | N |
| ATOM | 2664 | CA | ASN | B | 235 | −58.732 | 40.724 | 4.586 | 1.00 | 24.46 | C |
| ATOM | 2665 | CB | ASN | B | 235 | −57.626 | 41.759 | 4.857 | 1.00 | 24.48 | C |
| ATOM | 2666 | CG | ASN | B | 235 | −56.321 | 41.474 | 4.118 | 1.00 | 24.71 | C |
| ATOM | 2667 | OD1 | ASN | B | 235 | −55.513 | 42.383 | 3.923 | 1.00 | 25.10 | O |
| ATOM | 2668 | ND2 | ASN | B | 235 | −56.096 | 40.223 | 3.725 | 1.00 | 24.38 | O |
| ATOM | 2669 | C | ASN | B | 235 | −59.985 | 41.137 | 5.352 | 1.00 | 24.60 | C |
| ATOM | 2670 | O | ASN | B | 235 | −60.622 | 42.134 | 5.003 | 1.00 | 24.04 | O |
| ATOM | 2671 | N | TYR | B | 236 | −60.339 | 40.365 | 6.377 | 1.00 | 24.57 | N |
| ATOM | 2672 | CA | TYR | B | 236 | −61.441 | 40.707 | 7.270 | 1.00 | 24.69 | C |
| ATOM | 2673 | CB | TYR | B | 236 | −62.300 | 39.484 | 7.558 | 1.00 | 24.40 | C |
| ATOM | 2674 | CG | TYR | B | 236 | −63.100 | 39.041 | 6.359 | 1.00 | 23.91 | C |
| ATOM | 2675 | CD1 | TYR | B | 236 | −62.536 | 38.234 | 5.373 | 1.00 | 23.72 | C |
| ATOM | 2676 | CE1 | TYR | B | 236 | −63.272 | 37.826 | 4.268 | 1.00 | 23.66 | C |
| ATOM | 2677 | CZ | TYR | B | 236 | −64.591 | 38.231 | 4.135 | 1.00 | 23.77 | C |
| ATOM | 2678 | OH | TYR | B | 236 | −65.327 | 37.832 | 3.042 | 1.00 | 23.88 | O |
| ATOM | 2679 | CE2 | TYR | B | 236 | −65.174 | 39.038 | 5.099 | 1.00 | 23.67 | C |
| ATOM | 2680 | CD2 | TYR | B | 236 | −64.432 | 39.438 | 6.202 | 1.00 | 23.66 | C |
| ATOM | 2681 | C | TYR | B | 236 | −60.856 | 41.285 | 8.548 | 1.00 | 25.38 | C |
| ATOM | 2682 | O | TYR | B | 236 | −60.049 | 40.635 | 9.216 | 1.00 | 25.96 | O |
| ATOM | 2683 | N | MET | B | 237 | −61.292 | 42.499 | 8.887 | 1.00 | 25.88 | N |
| ATOM | 2684 | CA | MET | B | 237 | −60.593 | 43.363 | 9.842 | 1.00 | 26.36 | C |
| ATOM | 2685 | CB | MET | B | 237 | −60.518 | 44.774 | 9.244 | 1.00 | 25.39 | C |
| ATOM | 2686 | CG | MET | B | 237 | −59.819 | 44.861 | 7.896 | 1.00 | 25.84 | C |
| ATOM | 2687 | SD | MET | B | 237 | −58.183 | 44.119 | 7.886 | 1.00 | 24.75 | S |
| ATOM | 2688 | CE | MET | B | 237 | −57.398 | 44.956 | 7.264 | 1.00 | 24.80 | C |
| ATOM | 2689 | C | MET | B | 237 | −61.208 | 43.419 | 11.248 | 1.00 | 27.33 | C |
| ATOM | 2690 | O | MET | B | 237 | −60.834 | 44.268 | 12.062 | 1.00 | 27.26 | O |
| ATOM | 2691 | N | CYS | B | 238 | −62.120 | 42.498 | 11.542 | 1.00 | 28.83 | N |
| ATOM | 2692 | CA | CYS | B | 238 | −62.760 | 42.416 | 12.851 | 1.00 | 30.31 | C |
| ATOM | 2693 | CB | CYS | B | 238 | −64.017 | 43.287 | 12.855 | 1.00 | 31.55 | C |
| ATOM | 2694 | SG | CYS | B | 238 | −64.714 | 43.697 | 14.468 | 1.00 | 33.21 | S |
| ATOM | 2695 | C | CYS | B | 238 | −63.115 | 40.952 | 13.104 | 1.00 | 30.59 | C |
| ATOM | 2696 | O | CYS | B | 238 | −63.492 | 40.243 | 12.166 | 1.00 | 30.74 | O |
| ATOM | 2697 | N | ASN | B | 239 | −62.972 | 40.500 | 14.351 | 1.00 | 30.60 | N |
| ATOM | 2698 | CA | ASN | B | 239 | −63.375 | 39.136 | 14.730 | 1.00 | 31.29 | C |
| ATOM | 2699 | CB | ASN | B | 239 | −62.786 | 38.721 | 16.089 | 1.00 | 30.98 | C |
| ATOM | 2700 | CG | ASN | B | 239 | −61.340 | 38.263 | 15.988 | 1.00 | 31.01 | C |
| ATOM | 2701 | OD1 | ASN | B | 239 | −61.017 | 37.345 | 15.231 | 1.00 | 31.20 | O |
| ATOM | 2702 | ND2 | ASN | B | 239 | −60.464 | 38.889 | 16.765 | 1.00 | 30.84 | N |
| ATOM | 2703 | C | ASN | B | 239 | −64.899 | 39.026 | 14.760 | 1.00 | 31.89 | C |
| ATOM | 2704 | O | ASN | B | 239 | −65.603 | 40.033 | 14.855 | 1.00 | 31.50 | O |
| ATOM | 2705 | N | SER | B | 240 | −65.401 | 37.799 | 14.670 | 1.00 | 32.27 | N |
| ATOM | 2706 | CA | SER | B | 240 | −65.835 | 37.543 | 14.816 | 1.00 | 32.18 | C |
| ATOM | 2707 | CB | SER | B | 240 | −67.160 | 36.097 | 14.454 | 1.00 | 31.99 | C |
| ATOM | 2708 | OG | SER | B | 240 | −66.816 | 35.840 | 13.106 | 1.00 | 31.67 | O |
| ATOM | 2709 | C | SER | B | 240 | −67.317 | 37.851 | 16.236 | 1.00 | 32.65 | C |
| ATOM | 2710 | O | SER | B | 240 | −68.451 | 38.289 | 16.421 | 1.00 | 32.79 | O |
| ATOM | 2711 | N | SER | B | 241 | −66.447 | 37.629 | 17.224 | 1.00 | 33.40 | N |
| ATOM | 2712 | CA | SER | B | 241 | −66.749 | 37.929 | 18.628 | 1.00 | 33.76 | C |
| ATOM | 2713 | CB | SER | B | 241 | −65.702 | 37.286 | 19.549 | 1.00 | 33.87 | C |
| ATOM | 2714 | OG | SER | B | 241 | −64.400 | 37.773 | 19.276 | 1.00 | 33.42 | O |
| ATOM | 2715 | C | SER | B | 241 | −66.865 | 39.431 | 18.940 | 1.00 | 34.35 | C |
| ATOM | 2716 | O | SER | B | 241 | −67.455 | 39.801 | 19.959 | 1.00 | 34.31 | O |
| ATOM | 2717 | N | CYS | B | 242 | −66.292 | 40.284 | 18.089 | 1.00 | 34.26 | N |
| ATOM | 2718 | CA | CYS | B | 242 | −66.465 | 41.735 | 18.210 | 1.00 | 35.26 | C |
| ATOM | 2719 | CB | CYS | B | 242 | −65.680 | 42.475 | 17.127 | 1.00 | 35.54 | C |
| ATOM | 2720 | SG | CYS | B | 242 | −63.890 | 42.611 | 17.366 | 1.00 | 36.01 | S |
| ATOM | 2721 | C | CYS | B | 242 | −67.936 | 42.133 | 18.104 | 1.00 | 35.08 | C |
| ATOM | 2722 | O | CYS | B | 242 | −68.330 | 43.196 | 18.577 | 1.00 | 35.07 | O |
| ATOM | 2723 | N | ILE | B | 251 | −72.643 | 36.788 | 9.396 | 1.00 | 24.89 | N |
| ATOM | 2724 | CA | ILE | B | 251 | −72.221 | 37.688 | 8.326 | 1.00 | 24.81 | C |
| ATOM | 2725 | CB | ILE | B | 251 | −70.897 | 38.420 | 8.676 | 1.00 | 25.06 | C |
| ATOM | 2726 | CG1 | ILE | B | 251 | −71.012 | 39.139 | 10.026 | 1.00 | 25.23 | C |
| ATOM | 2727 | CD1 | ILE | B | 251 | −69.113 | 39.740 | 10.519 | 1.00 | 25.28 | C |
| ATOM | 2728 | CG2 | ILE | B | 251 | −70.527 | 39.438 | 7.595 | 1.00 | 24.99 | C |
| ATOM | 2729 | C | ILE | B | 251 | −72.044 | 36.920 | 7.008 | 1.00 | 24.64 | C |
| ATOM | 2730 | O | ILE | B | 251 | −71.288 | 35.950 | 6.950 | 1.00 | 24.25 | O |
| ATOM | 2731 | N | LEU | B | 252 | −72.756 | 37.362 | 5.969 | 1.00 | 24.59 | N |
| ATOM | 2732 | CA | LEU | B | 252 | −72.510 | 36.935 | 4.590 | 1.00 | 24.81 | C |
| ATOM | 2733 | CB | LEU | B | 252 | −73.815 | 36.818 | 3.798 | 1.00 | 24.31 | C |
| ATOM | 2734 | CG | LEU | B | 252 | −74.927 | 35.885 | 4.266 | 1.00 | 25.95 | C |
| ATOM | 2735 | CD1 | LEU | B | 252 | −75.960 | 35.758 | 3.152 | 1.00 | 26.15 | C |
| ATOM | 2736 | CD2 | LEU | B | 252 | −74.383 | 34.522 | 4.670 | 1.00 | 26.34 | C |
| ATOM | 2737 | C | LEU | B | 252 | −71.661 | 37.972 | 3.876 | 1.00 | 24.10 | C |
| ATOM | 2738 | O | LEU | B | 252 | −71.883 | 39.170 | 4.046 | 1.00 | 23.74 | O |
| ATOM | 2739 | N | THR | B | 253 | −70.709 | 37.502 | 3.072 | 1.00 | 23.12 | N |
| ATOM | 2740 | CA | THR | B | 253 | −70.048 | 38.332 | 2.071 | 1.00 | 22.35 | C |
| ATOM | 2741 | CB | THR | B | 253 | −65.554 | 37.993 | 1.945 | 1.00 | 22.16 | C |
| ATOM | 2742 | OG1 | THR | B | 253 | −67.947 | 38.072 | 3.233 | 1.00 | 22.26 | O |
| ATOM | 2743 | OG2 | THR | B | 253 | −67.842 | 38.967 | 1.011 | 1.00 | 21.99 | C |
| ATOM | 2744 | C | THR | B | 253 | −70.731 | 38.063 | 0.743 | 1.00 | 22.31 | C |
| ATOM | 2745 | O | THR | B | 253 | −70.896 | 36.902 | 0.355 | 1.00 | 22.47 | O |
| ATOM | 2746 | N | ILE | B | 254 | −71.131 | 39.136 | 0.062 | 1.00 | 21.95 | N |
| ATOM | 2747 | CA | ILE | B | 254 | −71.727 | 39.062 | −1.266 | 1.00 | 21.81 | C |
| ATOM | 2748 | CB | ILE | B | 254 | −73.049 | 39.867 | −1.344 | 1.00 | 21.91 | C |
| ATOM | 2749 | CG1 | ILE | B | 254 | −74.054 | 39.374 | −0.293 | 1.00 | 21.83 | C |
| ATOM | 2750 | CD1 | ILE | B | 254 | −75.198 | 40.334 | −0.038 | 1.00 | 21.97 | C |
| ATOM | 2751 | CG2 | ILE | B | 254 | −73.664 | 39.762 | −2.737 | 1.00 | 21.92 | C |
| ATOM | 2752 | C | ILE | B | 254 | −70.719 | 39.630 | −2.266 | 1.00 | 21.71 | C |
| ATOM | 2753 | O | ILE | B | 254 | −70.400 | 40.821 | −2.220 | 1.00 | 21.04 | O |
| ATOM | 2754 | N | ILE | B | 255 | −70.214 | 38.769 | −3.149 | 1.00 | 22.22 | N |
| ATOM | 2755 | CA | ILE | B | 255 | −69.353 | 39.185 | −4.256 | 1.00 | 22.62 | C |
| ATOM | 2756 | CB | ILE | B | 255 | −68.286 | 38.120 | −4.610 | 1.00 | 22.58 | C |
| ATOM | 2757 | CG1 | ILE | B | 255 | −67.494 | 37.688 | −3.364 | 1.00 | 22.32 | C |
| ATOM | 2758 | CD1 | ILE | B | 255 | −66.808 | 38.813 | −2.620 | 1.00 | 22.08 | C |
| ATOM | 2759 | CG2 | ILE | B | 255 | −67.340 | 38.637 | −5.695 | 1.00 | 22.56 | C |
| ATOM | 2760 | C | ILE | B | 255 | −70.239 | 39.440 | −5.470 | 1.00 | 23.74 | C |
| ATOM | 2761 | O | ILE | B | 255 | −70.895 | 38.521 | −5.962 | 1.00 | 24.13 | O |
| ATOM | 2762 | N | THR | B | 256 | −70.262 | 40.691 | −5.933 | 1.00 | 24.82 | N |
| ATOM | 2762 | CA | THR | B | 256 | −71.028 | 41.096 | −7.113 | 1.00 | 25.57 | C |
| ATOM | 2764 | CB | THR | B | 256 | −71.916 | 42.330 | −6.832 | 1.00 | 25.46 | C |
| ATOM | 2765 | OG1 | THR | B | 256 | −71.224 | 43.239 | −5.974 | 1.00 | 25.91 | O |
| ATOM | 2766 | CG2 | THR | B | 256 | −73.206 | 41.935 | −6.162 | 1.00 | 25.33 | C |
| ATOM | 2767 | C | THR | B | 256 | −70.085 | 41.446 | −8.256 | 1.00 | 26.41 | C |
| ATOM | 2768 | O | THR | B | 256 | −69.114 | 42.179 | −8.069 | 1.00 | 26.75 | O |
| ATOM | 2769 | N | LEU | B | 257 | −70.377 | 40.908 | −9.436 | 1.00 | 27.49 | N |
| ATOM | 2770 | CA | LEU | B | 257 | −69.681 | 41.282 | −10.655 | 1.00 | 28.46 | C |
| ATOM | 2771 | CB | LEU | B | 257 | −69.535 | 40.076 | −11.588 | 1.00 | 28.44 | C |
| ATOM | 2772 | CG | LEU | B | 257 | −68.241 | 40.079 | −12.406 | 1.00 | 28.50 | C |
| ATOM | 2773 | CD1 | LEU | B | 257 | −67.083 | 39.578 | −11.551 | 1.00 | 28.67 | C |
| ATOM | 2774 | CD2 | LEU | B | 257 | −68.380 | 39.236 | −13.663 | 1.00 | 28.64 | C |
| ATOM | 2775 | C | LEU | B | 257 | −70.501 | 42.387 | −11.317 | 1.00 | 29.30 | C |
| ATOM | 2776 | O | LEU | B | 257 | −71.716 | 42.242 | −11.491 | 1.00 | 29.54 | O |
| ATOM | 2777 | N | GLU | B | 258 | −69.836 | 43.489 | −11.662 | 1.00 | 30.07 | N |
| ATOM | 2778 | CA | GLU | B | 258 | −70.483 | 44.652 | −12.267 | 1.00 | 30.54 | C |
| ATOM | 2779 | CB | GLU | B | 258 | −70.455 | 45.844 | −11.313 | 1.00 | 30.74 | C |
| ATOM | 2780 | CG | GLU | B | 258 | −70.977 | 45.596 | −9.907 | 1.00 | 30.96 | C |
| ATOM | 2781 | CD | GLU | B | 258 | −70.572 | 46.693 | −8.933 | 1.00 | 31.13 | C |
| ATOM | 2782 | OE1 | GLU | B | 258 | −69.981 | 47.716 | −9.355 | 1.00 | 30.64 | O |
| ATOM | 2783 | OE2 | GLU | B | 258 | −70.843 | 46.530 | −7.728 | 1.00 | 31.18 | O |
| ATOM | 2784 | C | GLU | B | 258 | −69.731 | 45.077 | −13.512 | 1.00 | 31.43 | C |
| ATOM | 2785 | O | GLU | B | 258 | −68.504 | 44.990 | −13.549 | 1.00 | 31.38 | O |
| ATOM | 2786 | N | ASP | B | 259 | −70.452 | 45.577 | −14.512 | 1.00 | 32.36 | N |
| ATOM | 2787 | CA | ASP | B | 259 | −69.804 | 46.257 | −15.639 | 1.00 | 33.46 | C |
| ATOM | 2788 | CB | ASP | B | 259 | −70.781 | 46.488 | −16.816 | 1.00 | 34.21 | C |
| ATOM | 2789 | CG | ASP | B | 259 | −71.996 | 47.344 | −16.447 | 1.00 | 35.04 | C |
| ATOM | 2790 | OD1 | ASP | B | 259 | −72.015 | 47.868 | −15.316 | 1.00 | 36.05 | O |
| ATOM | 2791 | OD2 | ASP | B | 259 | −72.891 | 47.486 | −17.309 | 1.00 | 35.72 | O |
| ATOM | 2792 | C | ASP | B | 259 | −69.171 | 47.570 | −15.153 | 1.00 | 35.55 | C |
| ATOM | 2793 | O | ASP | B | 259 | −69.359 | 47.968 | −13.997 | 1.00 | 33.27 | O |
| ATOM | 2794 | N | SER | B | 260 | −68.431 | 48.237 | −16.033 | 1.00 | 33.59 | N |
| ATOM | 2795 | CA | SER | B | 260 | −67.788 | 49.519 | −15.711 | 1.00 | 33.59 | C |
| ATOM | 2796 | CB | SER | B | 260 | −66.969 | 50.013 | −16.908 | 1.00 | 34.14 | C |
| ATOM | 2797 | OG | SER | B | 260 | −67.753 | 50.016 | −18.088 | 1.00 | 34.37 | O |
| ATOM | 2798 | C | SER | B | 260 | −68.769 | 50.615 | −15.259 | 1.00 | 34.78 | C |
| ATOM | 2799 | O | SER | B | 260 | −68.371 | 51.534 | −14.544 | 1.00 | 34.27 | O |
| ATOM | 2800 | N | SER | B | 261 | −70.033 | 50.509 | −15.680 | 1.00 | 34.21 | N |
| ATOM | 2801 | CA | SER | B | 261 | −71.102 | 51.432 | −15.272 | 1.00 | 34.35 | C |
| ATOM | 2802 | CB | SER | B | 261 | −72.172 | 51.501 | −16.368 | 1.00 | 34.76 | C |
| ATOM | 2803 | OG | SER | B | 261 | −71.589 | 51.663 | −17.651 | 1.00 | 35.09 | O |
| ATOM | 2804 | C | SER | B | 261 | −71.790 | 51.091 | −13.938 | 1.00 | 34.98 | C |
| ATOM | 2805 | O | SER | B | 261 | −72.639 | 51.589 | −13.482 | 1.00 | 34.99 | O |
| ATOM | 2806 | N | GLY | B | 262 | −71.451 | 49.949 | −13.331 | 1.00 | 35.46 | N |
| ATOM | 2807 | CA | GLY | B | 262 | −72.001 | 49.545 | −12.027 | 1.00 | 35.60 | C |
| ATOM | 2808 | C | GLY | B | 262 | −73.226 | 48.642 | −12.041 | 1.00 | 35.74 | C |
| ATOM | 2809 | O | GLY | B | 262 | −73.739 | 48.295 | −10.976 | 1.00 | 36.65 | O |
| ATOM | 2810 | N | ASN | B | 263 | −73.688 | 48.248 | −13.229 | 1.00 | 35.61 | N |
| ATOM | 2811 | CA | ASN | B | 263 | −74.864 | 47.381 | −13.369 | 1.00 | 35.61 | C |
| ATOM | 2812 | CB | ASN | B | 263 | −75.484 | 47.514 | −14.766 | 1.00 | 36.17 | C |
| ATOM | 2813 | CG | ASN | B | 263 | −76.048 | 48.900 | −15.033 | 1.00 | 36.45 | C |
| ATOM | 2814 | OD1 | ASN | B | 263 | −77.261 | 49.100 | −15.006 | 1.00 | 37.19 | O |
| ATOM | 2815 | ND2 | ASN | B | 263 | −75.171 | 49.860 | −15.301 | 1.00 | 36.97 | N |
| ATOM | 2816 | C | ASN | B | 263 | −74.493 | 45.920 | −13.117 | 1.00 | 34.52 | C |
| ATOM | 2817 | O | ASN | B | 263 | −73.389 | 45.492 | −13.450 | 1.00 | 35.12 | O |
| ATOM | 2818 | N | LEU | B | 264 | −75.435 | 45.163 | −12.559 | 1.00 | 33.56 | N |
| ATOM | 2819 | CA | LEU | B | 264 | −75.210 | 43.865 | −12.157 | 1.00 | 32.99 | C |
| ATOM | 2820 | CB | LEU | B | 264 | −76.416 | 43.259 | −11.353 | 1.00 | 33.46 | C |
| ATOM | 2821 | CG | LEU | B | 264 | −76.364 | 41.846 | −10.754 | 1.00 | 33.78 | C |
| ATOM | 2822 | CD1 | LEU | B | 264 | −75.432 | 41.797 | −9.553 | 1.00 | 33.88 | C |
| ATOM | 2823 | CD2 | LEU | B | 264 | −77.760 | 41.380 | −10.368 | 1.00 | 34.02 | C |
| ATOM | 2824 | C | LEU | B | 264 | −74.974 | 42.815 | −13.338 | 1.00 | 32.07 | C |
| ATOM | 2825 | O | LEU | B | 264 | −75.835 | 42.677 | −14.205 | 1.00 | 31.46 | O |
| ATOM | 2826 | N | LEU | B | 265 | −73.806 | 42.168 | −13.353 | 1.00 | 31.42 | N |
| ATOM | 2827 | CA | LEU | B | 265 | −73.523 | 41.047 | −14.264 | 1.00 | 30.85 | C |
| ATOM | 2828 | CB | LEU | B | 265 | −72.101 | 41.146 | −14.821 | 1.00 | 30.51 | C |
| ATOM | 2829 | CG | LEU | B | 265 | −71.720 | 42.422 | −15.572 | 1.00 | 30.14 | C |
| ATOM | 2830 | CD1 | LEU | B | 265 | −70.297 | 42.291 | −16.086 | 1.00 | 30.19 | C |
| ATOM | 2831 | CD2 | LEU | B | 265 | −72.679 | 42.716 | −16.717 | 1.00 | 30.25 | C |
| ATOM | 2832 | C | LEU | B | 265 | −73.704 | 39.688 | −13.575 | 1.00 | 30.73 | C |
| ATOM | 2833 | O | LEU | B | 265 | −74.208 | 38.748 | −14.193 | 1.00 | 31.47 | O |
| ATOM | 2834 | N | GLY | B | 266 | −73.276 | 39.583 | −12.315 | 1.00 | 29.69 | N |
| ATOM | 2835 | CA | GLY | B | 266 | −73.427 | 38.351 | −11.535 | 1.00 | 29.18 | C |
| ATOM | 2836 | C | GLY | B | 266 | −73.362 | 38.575 | −10.033 | 1.00 | 28.88 | C |
| ATOM | 2837 | O | GLY | B | 266 | −72.942 | 39.641 | −9.571 | 1.00 | 29.06 | O |
| ATOM | 2838 | N | ARG | B | 267 | −73.782 | 37.565 | −9.275 | 1.00 | 28.17 | N |
| ATOM | 2839 | CA | ARG | B | 267 | −73.764 | 37.611 | −7.813 | 1.00 | 27.69 | C |
| ATOM | 2840 | CB | ARG | B | 267 | −75.040 | 38.266 | −7.266 | 1.00 | 27.71 | C |
| ATOM | 2841 | CG | ARG | B | 267 | −74.926 | 38.691 | −5.806 | 1.00 | 27.72 | C |
| ATOM | 2842 | CD | ARG | B | 267 | −76.253 | 39.094 | −5.177 | 1.00 | 27.88 | C |
| ATOM | 2843 | BE | ARG | B | 267 | −76.919 | 40.217 | −5.848 | 1.00 | 28.17 | N |
| ATOM | 2844 | CZ | ARG | B | 267 | −76.010 | 40.150 | −6.622 | 1.00 | 28.38 | C |
| ATOM | 2845 | NH1 | ARG | B | 267 | −76.630 | 38.998 | −6.868 | 1.00 | 28.55 | N |
| ATOM | 2846 | NH2 | ARG | B | 267 | −78.497 | 41.273 | −7.149 | 1.00 | 28.83 | N |
| ATOM | 2847 | C | ARG | B | 267 | −73.606 | 36.214 | −7.210 | 1.00 | 27.40 | C |
| ATOM | 2848 | O | ARG | B | 267 | −74.286 | 35.267 | −7.613 | 1.00 | 27.61 | O |
| ATOM | 2849 | N | ASN | B | 268 | −72.698 | 36.103 | −6.243 | 1.00 | 26.93 | N |
| ATOM | 2850 | CA | ASN | B | 268 | −72.572 | 34.917 | −5.397 | 1.00 | 26.51 | C |
| ATOM | 2851 | CB | ASN | B | 268 | −71.441 | 34.017 | −5.902 | 1.00 | 26.53 | C |
| ATOM | 2852 | CG | ASN | B | 268 | −71.884 | 33.090 | −7.023 | 1.00 | 26.77 | C |
| ATOM | 2853 | OD1 | ASN | B | 268 | −72.599 | 32.117 | −6.786 | 1.00 | 26.99 | O |
| ATOM | 2854 | ND2 | ASN | B | 268 | −71.449 | 33.379 | −8.248 | 1.00 | 26.35 | N |
| ATOM | 2855 | C | ASN | B | 268 | −72.327 | 35.372 | −3.958 | 1.00 | 26.00 | C |
| ATOM | 2856 | O | ASN | B | 268 | −72.069 | 36.557 | −3.709 | 1.00 | 25.82 | O |
| ATOM | 2857 | N | SER | B | 269 | −72.431 | 34.439 | −3.014 | 1.00 | 25.19 | N |
| ATOM | 2858 | CA | SER | B | 269 | −72.218 | 34.758 | −1.605 | 1.00 | 24.84 | C |
| ATOM | 2859 | CB | SER | B | 269 | −73.487 | 35.366 | −0.995 | 1.00 | 24.79 | C |
| ATOM | 2860 | OG | SER | B | 269 | −74.532 | 34.412 | −0.920 | 1.00 | 24.47 | O |
| ATOM | 2861 | C | SER | B | 269 | −71.762 | 33.571 | −0.765 | 1.00 | 24.44 | C |
| ATOM | 2862 | O | SER | B | 269 | −71.960 | 32.414 | −1.129 | 1.00 | 24.72 | O |
| ATOM | 2863 | N | PHE | B | 270 | −71.143 | 33.891 | 0.365 | 1.00 | 24.11 | N |
| ATOM | 2864 | CA | PHE | B | 270 | −70.681 | 32.901 | 1.322 | 1.00 | 23.63 | C |
| ATOM | 2865 | CB | PHE | B | 270 | −69.285 | 32.392 | 0.941 | 1.00 | 23.64 | C |
| ATOM | 2866 | CG | PHE | B | 270 | −68.233 | 33.469 | 0.856 | 1.00 | 23.25 | C |
| ATOM | 2867 | CD1 | PHE | B | 270 | −68.035 | 34.181 | −0.324 | 1.00 | 23.36 | C |
| ATOM | 2868 | CE1 | PHE | B | 270 | −67.059 | 35.167 | −0.404 | 1.00 | 23.07 | C |
| ATOM | 2869 | CZ | PHE | B | 270 | −66.264 | 35.446 | 0.699 | 1.00 | 23.04 | C |
| ATOM | 2870 | CE2 | PHE | B | 270 | −66.447 | 34.740 | 1.878 | 1.00 | 23.09 | C |
| ATOM | 2871 | CD2 | PHE | B | 270 | −67.423 | 33.756 | 1.949 | 1.00 | 23.17 | C |
| ATOM | 2872 | C | PHE | B | 270 | −70.675 | 33.506 | 2.716 | 1.00 | 23.42 | C |
| ATOM | 2873 | O | PHE | B | 270 | −70.441 | 34.711 | 2.874 | 1.00 | 23.86 | O |
| ATOM | 2874 | N | GLU | B | 271 | −70.943 | 32.676 | 3.720 | 1.00 | 23.07 | N |
| ATOM | 2875 | CA | GLU | B | 271 | −70.872 | 33.100 | 5.115 | 1.00 | 23.11 | C |
| ATOM | 2876 | CB | GLU | B | 271 | −71.572 | 32.079 | 6.022 | 1.00 | 23.92 | C |
| ATOM | 2877 | CG | GLU | B | 271 | −71−781 | 32.548 | 7.460 | 1.00 | 24.58 | C |
| ATOM | 2878 | CD | GLU | B | 271 | −72.584 | 31.568 | 8.305 | 1.00 | 25.49 | C |
| ATOM | 2879 | OE1 | GLU | B | 271 | −73.557 | 30.969 | 7.795 | 1.00 | 25.92 | O |
| ATOM | 2880 | OE2 | GLU | B | 271 | −72.245 | 31.403 | 9.496 | 1.00 | 26.74 | O |
| ATOM | 2881 | C | GLU | B | 271 | −69.407 | 33.252 | 5.513 | 1.00 | 22.35 | C |
| ATOM | 2882 | O | GLU | B | 271 | −68.539 | 32.605 | 4.932 | 1.00 | 21.13 | O |
| ATOM | 2883 | N | VAL | B | 272 | −69.137 | 34.124 | 6.481 | 1.00 | 22.04 | N |
| ATOM | 2884 | CA | VAL | B | 272 | −67.801 | 34.240 | 7.065 | 1.00 | 22.39 | C |
| ATOM | 2885 | CB | VAL | B | 272 | −67.110 | 35.591 | 6.767 | 1.00 | 22.71 | C |
| ATOM | 2886 | CG1 | VAL | B | 272 | −65.593 | 35.424 | 6.766 | 1.00 | 22.60 | C |
| ATOM | 2887 | CG2 | VAL | B | 272 | −67.583 | 36.176 | 5.452 | 1.00 | 22.84 | C |
| ATOM | 2868 | C | VAL | B | 272 | −67.882 | 34.133 | 8.574 | 1.00 | 22.89 | C |
| ATOM | 2889 | O | VAL | B | 272 | −68.878 | 34.532 | 9.188 | 1.00 | 23.24 | O |
| ATOM | 2890 | N | ARG | B | 273 | −66.819 | 33.591 | 9.155 | 1.00 | 23.06 | N |
| ATOM | 2891 | CA | ARG | B | 273 | −66.542 | 33.730 | 10.568 | 1.00 | 23.66 | C |
| ATOM | 2892 | CB | ARG | B | 273 | −66.850 | 32.435 | 11.319 | 1.00 | 23.66 | C |
| ATOM | 2893 | CG | ARG | B | 273 | −66.237 | 32.390 | 12.705 | 1.00 | 23.56 | C |
| ATOM | 2894 | CD | ARG | B | 273 | −66.730 | 31.222 | 13.530 | 1.00 | 23.56 | C |
| ATOM | 2895 | NE | ARG | B | 273 | −65.997 | 31.182 | 14.790 | 1.00 | 23.67 | N |
| ATOM | 2896 | CZ | ARG | B | 273 | −66.210 | 31.991 | 15.831 | 1.00 | 24.10 | C |
| ATOM | 2897 | NH1 | ARG | B | 273 | −67.155 | 32.937 | 15.802 | 1.00 | 24.18 | N |
| ATOM | 2898 | NH2 | ARG | B | 273 | −65.462 | 31.857 | 16.925 | 1.00 | 24.18 | N |
| ATOM | 2899 | C | ARG | B | 273 | −65.067 | 34.090 | 10.715 | 1.00 | 24.00 | C |
| ATOM | 2900 | O | ARG | B | 273 | −64.204 | 33.372 | 10.214 | 1.00 | 23.46 | O |
| ATOM | 2901 | N | VAL | B | 274 | −64.791 | 35.191 | 11.407 | 1.00 | 25.13 | N |
| ATOM | 2902 | CA | VAL | B | 274 | −63.423 | 35.616 | 11.698 | 1.00 | 25.93 | C |
| ATOM | 2903 | CB | VAL | B | 274 | −63.255 | 37.140 | 11.535 | 1.00 | 26.01 | C |
| ATOM | 2904 | CG1 | VAL | B | 274 | −61.785 | 37.536 | 11.651 | 1.00 | 25.84 | C |
| ATOM | 2905 | CG2 | VAL | B | 274 | −63.830 | 37.601 | 10.202 | 1.00 | 25.76 | C |
| ATOM | 2906 | C | VAL | B | 274 | −63.087 | 35.1.79 | 13.125 | 1.00 | 26.74 | C |
| ATOM | 2907 | O | VAL | B | 274 | −63.788 | 35.548 | 14.072 | 1.00 | 27.66 | O |
| ATOM | 2906 | N | CYS | B | 275 | −62.021 | 34.391 | 13.266 | 1.00 | 27.46 | N |
| ATOM | 2909 | CA | CYS | B | 275 | −61.685 | 33.721 | 14.526 | 1.00 | 28.04 | C |
| ATOM | 2910 | CB | CYS | B | 275 | −62.580 | 32.497 | 14.714 | 1.00 | 27.87 | C |
| ATOM | 2911 | SG | CYS | B | 275 | −62.434 | 31.279 | 13.384 | 1.00 | 28.62 | S |
| ATOM | 2912 | C | CYS | B | 275 | −60.226 | 33.276 | 14.528 | 1.00 | 28.34 | C |
| ATOM | 2913 | O | CYS | B | 275 | −59.666 | 32.997 | 13.473 | 1.00 | 29.71 | O |
| ATOM | 2914 | N | ALA | B | 276 | −59.627 | 33.180 | 15.711 | 1.00 | 28.51 | N |
| ATOM | 2915 | CA | ALA | B | 276 | −58.204 | 32.816 | 15.838 | 1.00 | 28.63 | C |
| ATOM | 2916 | CB | ALA | B | 276 | −57.697 | 33.148 | 17.232 | 1.00 | 28.70 | C |
| ATOM | 2917 | C | ALA | B | 276 | −57.891 | 31.349 | 15.496 | 1.00 | 28.94 | C |
| ATOM | 2918 | O | ALA | B | 276 | −56.772 | 31.048 | 15.073 | 1.00 | 28.82 | O |
| ATOM | 2919 | N | CYS | B | 277 | −58.867 | 30.454 | 15.860 | 1.00 | 29.34 | N |
| ATOM | 2920 | CA | CYS | B | 277 | −58.729 | 29.024 | 15.352 | 1.00 | 29.69 | C |
| ATOM | 2921 | CB | CYS | B | 277 | −59.000 | 28.161 | 16.595 | 1.00 | 30.45 | C |
| ATOM | 2922 | SG | CYS | B | 277 | −57.724 | 28.207 | 17.869 | 1.00 | 33.00 | S |
| ATOM | 2923 | C | CYS | B | 277 | −59.702 | 28.618 | 14.233 | 1.00 | 28.86 | C |
| ATOM | 2924 | O | CYS | B | 277 | −60.704 | 27.950 | 14.502 | 1.00 | 29.04 | O |
| ATOM | 2925 | N | PRO | B | 278 | −59.410 | 29.014 | 12.975 | 1.00 | 27.95 | N |
| ATOM | 2926 | CA | PRO | B | 278 | −60.341 | 28.678 | 11.876 | 1.00 | 27.88 | C |
| ATOM | 2927 | CB | PRO | B | 278 | −59.765 | 29.452 | 10.683 | 1.00 | 27.91 | C |
| ATOM | 2928 | CG | PRO | B | 278 | −58.329 | 29.639 | 11.012 | 1.00 | 27.93 | C |
| ATOM | 2929 | CD | PRO | B | 278 | −58.289 | 29.832 | 12.497 | 1.00 | 27.99 | C |
| ATOM | 2930 | C | PRO | B | 278 | −60.437 | 27.175 | 11.560 | 1.00 | 27.74 | C |
| ATOM | 2931 | O | PRO | B | 278 | −61.521 | 26.691 | 11.221 | 1.00 | 27.58 | O |
| ATOM | 2932 | N | GLY | B | 279 | −59.320 | 26.458 | 11.671 | 1.00 | 27.71 | N |
| ATOM | 2933 | CA | GLY | B | 279 | −59.289 | 25.016 | 11.435 | 1.00 | 27.84 | C |
| ATOM | 2934 | C | GLY | B | 279 | −60.163 | 24.239 | 12.402 | 1.00 | 28.30 | C |
| ATOM | 2935 | O | GLY | B | 279 | −60.988 | 23.424 | 11.960 | 1.00 | 28.32 | O |
| ATOM | 2936 | N | ARG | B | 280 | −60.000 | 24.501 | 13.698 | 1.00 | 28.70 | N |
| ATOM | 2937 | CA | ARG | B | 280 | −60.801 | 23.814 | 14.715 | 1.00 | 29.81 | C |
| ATOM | 2938 | CB | ARG | B | 280 | −60.271 | 24.073 | 16.129 | 1.00 | 31.09 | C |
| ATOM | 2939 | CG | ARG | B | 280 | −60.864 | 23.127 | 17.168 | 1.00 | 32.27 | C |
| ATOM | 2940 | CD | ARG | B | 280 | −60.264 | 23.333 | 18.547 | 1.00 | 33.56 | C |
| ATOM | 2941 | NE | ARG | B | 280 | −60.606 | 24.641 | 19.108 | 1.00 | 34.70 | N |
| ATOM | 2942 | CZ | ARG | B | 280 | −60.227 | 25.081 | 20.309 | 1.00 | 35.83 | C |
| ATOM | 2943 | NH1 | ARG | B | 280 | −59.481 | 24.326 | 21.116 | 1.00 | 36.62 | N |
| ATOM | 2944 | NH2 | ARG | B | 280 | −60.598 | 26.295 | 20.710 | 1.00 | 36.67 | N |
| ATOM | 2945 | C | ARG | B | 280 | −62.278 | 24.199 | 14.631 | 1.00 | 28.90 | C |
| ATOM | 2946 | O | ARG | B | 280 | −63.144 | 23.322 | 14.685 | 1.00 | 28.43 | O |
| ATOM | 2947 | N | ASP | B | 281 | −62.555 | 25.499 | 14.495 | 1.00 | 27.97 | N |
| ATOM | 2948 | CA | ASP | B | 281 | −63.937 | 26.003 | 14.427 | 1.00 | 27.48 | C |
| ATOM | 2949 | CB | ASP | B | 281 | −63.972 | 27.541 | 14.381 | 1.00 | 27.51 | C |
| ATOM | 2950 | CG | ASP | B | 281 | −63.635 | 28.185 | 15.719 | 1.00 | 27.50 | C |
| ATOM | 2951 | OD1 | ASP | B | 281 | −63.526 | 27.473 | 16.741 | 1.00 | 27.61 | O |
| ATOM | 2952 | OD2 | ASP | B | 281 | −63.483 | 29.423 | 15.751 | 1.00 | 27.46 | O |
| ATOM | 2953 | C | ASP | B | 281 | −64.727 | 25.446 | 13.247 | 1.00 | 27.16 | C |
| ATOM | 2954 | O | ASP | B | 281 | −65.916 | 25.171 | 13.384 | 1.00 | 27.48 | O |
| ATOM | 2955 | N | ARG | B | 282 | −64.073 | 25.293 | 12.096 | 1.00 | 26.30 | N |
| ATOM | 2956 | CA | ARG | B | 282 | −64.703 | 24.656 | 10.939 | 1.00 | 25.54 | C |
| ATOM | 2957 | CB | ARG | B | 282 | −63.804 | 24.745 | 9.699 | 1.00 | 25.61 | C |
| ATOM | 2958 | CG | ARG | B | 282 | −64.492 | 24.289 | 8.419 | 1.00 | 25.63 | C |
| ATOM | 2959 | CD | ARG | B | 282 | −63.543 | 24.161 | 7.234 | 1.00 | 25.79 | C |
| ATOM | 2960 | NE | ARG | B | 282 | −63.892 | 23.006 | 6.401 | 1.00 | 25.61 | N |
| ATOM | 2961 | CZ | ARG | B | 282 | −63.110 | 22.462 | 5.467 | 1.00 | 25.78 | C |
| ATOM | 2962 | NH1 | ARG | B | 282 | −61.906 | 22.960 | 5.192 | 1.00 | 26.00 | N |
| ATOM | 2963 | NH2 | ARG | B | 282 | −63.546 | 21.405 | 4.786 | 1.00 | 26.02 | N |
| ATOM | 2964 | C | ARG | B | 282 | −65.036 | 23.195 | 11.244 | 1.00 | 24.86 | C |
| ATOM | 2965 | O | ARG | B | 282 | −66.149 | 22.739 | 10.974 | 1.00 | 24.38 | O |
| ATOM | 2966 | N | ARG | B | 283 | −64.069 | 22.478 | 11.810 | 1.00 | 24.56 | N |
| ATOM | 2967 | CA | ARG | B | 283 | −64.236 | 21.056 | 12.129 | 1.00 | 24.79 | C |
| ATOM | 2968 | CB | ARG | B | 283 | −62.919 | 20.452 | 12.643 | 1.00 | 24.43 | C |
| ATOM | 2969 | CG | ARG | B | 283 | −62.823 | 18.946 | 12.450 | 1.00 | 24.32 | C |
| ATOM | 2970 | CD | ARG | B | 283 | −61.582 | 18.368 | 13.109 | 1.00 | 24.20 | C |
| ATOM | 2971 | NE | ARG | B | 283 | −60.344 | 18.818 | 12.474 | 1.00 | 23.85 | N |
| ATOM | 2972 | CZ | ARG | B | 283 | −59.122 | 18.384 | 12.788 | 1.00 | 23.57 | C |
| ATOM | 2973 | NH1 | ARG | B | 283 | −58.066 | 18.368 | 12.140 | 1.00 | 23.34 | N |
| ATOM | 2974 | NH2 | ARG | B | 283 | −58.938 | 17.468 | 13.379 | 1.00 | 23.37 | N |
| ATOM | 2975 | C | ARG | B | 283 | −65.363 | 20.830 | 13.147 | 1.00 | 25.29 | C |
| ATOM | 2976 | O | ARG | B | 283 | −66.172 | 19.914 | 12.984 | 1.00 | 25.05 | O |
| ATOM | 2977 | N | THR | B | 284 | −65.417 | 21.679 | 14.175 | 1.00 | 26.04 | N |
| ATOM | 2978 | CA | THR | B | 284 | −66.455 | 21.596 | 15.215 | 1.00 | 26.89 | C |
| ATOM | 2979 | CB | THR | B | 284 | −66.074 | 22.420 | 16.464 | 1.00 | 26.56 | C |
| ATOM | 2980 | OG1 | THR | B | 284 | −64.732 | 22.102 | 16.856 | 1.00 | 26.61 | O |
| ATOM | 2981 | CG2 | THR | B | 284 | −67.023 | 22.126 | 17.637 | 1.00 | 26.69 | C |
| ATOM | 2982 | C | THR | B | 284 | −67.834 | 22.044 | 14.715 | 1.00 | 27.73 | C |
| ATOM | 2983 | O | THR | B | 284 | −68.581 | 21.499 | 15.147 | 1.00 | 27.95 | O |
| ATOM | 2984 | N | GLU | B | 285 | −67.875 | 23.043 | 13.831 | 1.00 | 28.81 | N |
| ATOM | 2985 | CA | GLU | B | 285 | −69.140 | 23.448 | 13.204 | 1.00 | 29.61 | C |
| ATOM | 2986 | CB | GLU | B | 285 | −69.010 | 24.779 | 12.450 | 1.00 | 29.54 | C |
| ATOM | 2987 | CG | GLU | B | 285 | −69.052 | 25.999 | 13.367 | 1.00 | 29.76 | C |
| ATOM | 2988 | CD | GLU | B | 285 | −69.380 | 27.300 | 12.652 | 1.00 | 29.47 | C |
| ATOM | 2989 | OE1 | GLU | B | 285 | −70.291 | 27.307 | 11.800 | 1.00 | 28.87 | O |
| ATOM | 2990 | OE2 | GLU | B | 285 | −68.738 | 28.330 | 12.958 | 1.00 | 29.79 | O |
| ATOM | 2991 | C | GLU | B | 285 | −69.673 | 22.351 | 12.283 | 1.00 | 30.64 | C |
| ATOM | 2992 | O | GLU | B | 285 | −70.876 | 22.089 | 12.269 | 1.00 | 30.88 | O |
| ATOM | 2993 | N | GLU | B | 286 | −68.774 | 21.712 | 11.534 | 1.00 | 31.54 | N |
| ATOM | 2994 | CA | GLU | B | 286 | −69.132 | 20.567 | 10.685 | 1.00 | 32.36 | C |
| ATOM | 2995 | CB | GLU | B | 286 | −67.988 | 20.219 | 9.720 | 1.00 | 31.80 | C |
| ATOM | 2996 | CG | GLU | B | 286 | −67.884 | 21.190 | 8.551 | 1.00 | 31.31 | C |
| ATOM | 2997 | CD | GLU | B | 286 | −66.672 | 20.964 | 7.662 | 1.00 | 30.84 | C |
| ATOM | 2998 | OE1 | GLU | B | 286 | −65.841 | 20.076 | 7.952 | 1.00 | 31.62 | O |
| ATOM | 2999 | OE2 | GLU | B | 286 | −66.547 | 21.689 | 6.656 | 1.00 | 30.18 | O |
| ATOM | 3000 | C | GLU | B | 286 | −69.549 | 19.340 | 11.504 | 1.00 | 33.60 | C |
| ATOM | 3001 | O | GLU | B | 286 | −70.382 | 18.555 | 11.052 | 1.00 | 33.26 | O |
| ATOM | 3002 | N | GLU | B | 287 | −68.962 | 19.181 | 12.692 | 1.00 | 35.57 | N |
| ATOM | 3003 | CA | GLU | B | 287 | −69.406 | 18.169 | 13.666 | 1.00 | 37.79 | C |
| ATOM | 3004 | CB | GLU | B | 287 | −68.468 | 18.153 | 14.898 | 1.00 | 38.44 | C |
| ATOM | 3005 | CG | GLU | B | 287 | −68.982 | 17.478 | 16.174 | 1.00 | 39.37 | C |
| ATOM | 3006 | CD | GLU | B | 287 | −69.083 | 15.966 | 16.072 | 1.00 | 40.47 | C |
| ATOM | 3007 | OE1 | GLU | B | 287 | −69.684 | 15.459 | 15.101 | 1.00 | 41.13 | O |
| ATOM | 3008 | OE2 | GLU | B | 287 | −68.572 | 15.276 | 16.983 | 1.00 | 41.23 | O |
| ATOM | 3009 | C | GLU | B | 287 | −70.873 | 18.377 | 14.080 | 1.00 | 38.77 | C |
| ATOM | 3010 | O | GLU | B | 287 | −71.643 | 17.415 | 14.139 | 1.00 | 39.68 | O |
| ATOM | 3011 | N | ASN | B | 288 | −71.246 | 19.628 | 14.350 | 1.00 | 39.53 | N |
| ATOM | 3012 | CA | ASN | B | 288 | −72.611 | 19.974 | 14.781 | 1.00 | 39.40 | C |
| ATOM | 301.3 | CB | ASN | B | 288 | −72.682 | 21.454 | 15.196 | 1.00 | 38.86 | C |
| ATOM | 3014 | CG | ASN | B | 288 | −73.895 | 21.768 | 16.058 | 1.00 | 38.77 | C |
| ATOM | 3015 | OD1 | ASN | B | 288 | −74.113 | 21.140 | 17.095 | 1.00 | 38.20 | O |
| ATOM | 3016 | ND2 | ASN | B | 288 | −74.681 | 22.757 | 15.642 | 1.00 | 38.12 | N |
| ATOM | 3017 | C | ASN | B | 288 | −73.683 | 19.691 | 13.715 | 1.00 | 40.07 | C |
| ATOM | 3018 | O | ASN | B | 288 | −74.825 | 19.379 | 14.058 | 1.00 | 40.36 | O |
| ATOM | 3019 | N | LEU | B | 289 | −73.308 | 19.802 | 12.439 | 1.00 | 40.69 | N |
| ATOM | 3020 | CA | LEU | B | 289 | −74.236 | 19.625 | 11.315 | 1.00 | 41.08 | C |
| ATOM | 3021 | CB | LEU | B | 289 | −73.534 | 19.976 | 9.995 | 1.00 | 41.24 | C |
| ATOM | 3022 | CG | LEU | B | 289 | −74.340 | 19.942 | 8.692 | 1.00 | 41.40 | C |
| ATOM | 3023 | CD1 | LEU | B | 289 | −75.533 | 20.885 | 8.743 | 1.00 | 41.65 | C |
| ATOM | 3024 | CD2 | LEU | B | 289 | −73.440 | 20.283 | 7.514 | 1.00 | 41.18 | C |
| ATOM | 3025 | C | LEU | B | 289 | −74.788 | 18.203 | 11.244 | 1.00 | 41.57 | C |
| ATOM | 3026 | O | LEU | B | 289 | −75.954 | 17.999 | 10.904 | 1.00 | 42.31 | O |
| TER | 3027 | | LEU | B | 289 | | | | | | |
| HETATM | 3028 | ZN | ZN | C | 1 | −70.362 | 52.857 | 26.815 | 1.00 | 21.87 | ZN |
| HETATM | 3029 | ZN | ZN | C | 2 | −63.160 | 44.431 | 16.074 | 1.00 | 34.71 | ZN |
| HETATM | 3030 | AS | ARS | D | 1 | −79.922 | 62.501 | 33.936 | 1.00 | 31.59 | AS |
| HETATM | 3031 | AS | ARS | D | 2 | −60.144 | 33.454 | 4.974 | 1.00 | 42.70 | AS |
| HETATM | 3032 | O | HOH | E | 1 | −89.664 | 52.225 | 33.209 | 1.00 | 5.08 | O |
| HETATM | 3033 | O | HOH | E | 2 | −71.209 | 47.481 | 5.203 | 1.00 | 13.47 | O |
| HETATM | 3034 | O | HOH | E | 3 | −59.170 | 38.048 | 7.109 | 1.00 | 18.65 | O |
| HETATM | 3035 | O | HOH | E | 4 | −69.506 | 45.733 | 3.833 | 1.00 | 10.05 | O |
| HETATM | 3036 | O | HOH | E | 5 | −91.753 | 43.624 | 29.693 | 1.00 | 11.54 | O |
| HETATM | 3037 | O | HOH | E | 6 | −67.587 | 51.926 | −5.930 | 1.00 | 15.36 | O |
| HETATM | 3038 | O | HOH | E | 7 | −105.122 | 56.277 | 26.173 | 1.00 | 25.77 | O |
| HETATM | 3039 | O | HOH | E | 8 | −76.695 | 43.772 | 7.248 | 1.00 | 18.79 | O |
| HETATM | 3040 | O | HOH | E | 9 | −82.745 | 48.530 | 22.988 | 1.00 | 17.98 | O |
| HETATM | 3041 | O | HOH | E | 10 | −88.007 | 45.586 | 24.751 | 1.00 | 8.79 | O |
| HETATM | 3042 | O | HOH | E | 11 | −77.628 | 47.212 | 32.538 | 1.00 | 10.03 | O |
| HETATM | 3043 | O | HOH | E | 12 | −77.784 | 58.342 | 34.542 | 1.00 | 22.09 | O |
| HETATM | 3044 | O | HOH | E | 13 | −85.435 | 65.894 | 35.396 | 1.00 | 14.76 | O |
| HETATM | 3045 | O | HOH | E | 14 | −99.770 | 44.880 | 36.154 | 1.00 | 15.31 | O |
| HETATM | 3046 | O | HOH | E | 15 | −94.409 | 54.447 | 39.200 | 1.00 | 16.72 | O |
| HETATM | 3047 | O | HOH | E | 16 | −55.141 | 38.993 | −16.337 | 1.00 | 25.78 | O |
| HETATM | 3048 | O | HOH | E | 17 | −81.942 | 50.027 | 27.694 | 1.00 | 10.34 | O |
| HETATM | 3049 | O | HOH | E | 18 | −92.746 | 61.361 | 21.980 | 1.00 | 22.28 | O |
| HETATM | 3050 | O | HOH | E | 19 | −77.651 | 41.505 | 28.779 | 1.00 | 25.73 | O |
| HETATM | 3051 | O | HOH | E | 20 | −82.761 | 47.894 | 38.601 | 1.00 | 21.72 | O |
| HETATM | 3052 | O | HOH | E | 21 | −60.708 | 21.830 | 2.998 | 1.00 | 11.83 | O |
| HETATM | 3053 | O | HOH | E | 22 | −90.094 | 45.436 | 23.191 | 1.00 | 17.09 | O |
| HETATM | 3054 | O | HOH | E | 23 | −66.806 | 46.093 | 4.465 | 1.00 | 15.00 | O |
| HETATM | 3055 | O | HOH | E | 24 | −91.333 | 52.592 | 25.599 | 1.00 | 10.38 | O |
| HETATM | 3056 | O | HOH | E | 25 | −82.207 | 73.887 | 34.170 | 1.00 | 5.81 | O |
| HETATM | 3057 | O | HOH | E | 26 | −56.748 | 27.080 | 12.461 | 1.00 | 21.95 | O |
| HETATM | 3058 | O | HOH | E | 27 | −109.583 | 61.868 | 28.438 | 1.00 | 23.08 | O |
| HETATM | 3059 | O | HOH | E | 28 | −82.976 | 67.020 | 35.610 | 1.00 | 26.10 | O |
| HETATM | 3060 | O | HOH | E | 29 | −61.086 | 54.146 | −2.330 | 1.00 | 15.15 | O |
| HETATM | 3061 | O | HOH | E | 30 | −82.077 | 45.396 | 11.678 | 1.00 | 44.01 | O |
| HETATM | 3062 | O | HOH | E | 31 | −95.655 | 67.313 | 42.943 | 1.00 | 22.22 | O |
| HETATM | 3063 | O | HOH | E | 32 | −76.960 | 50.357 | 10.253 | 1.00 | 22.79 | O |
| HETATM | 3064 | O | HOH | E | 33 | −67.847 | 47.437 | 6.572 | 1.00 | 13.65 | O |
| HETATM | 3065 | O | HOH | E | 34 | −98.494 | 60.603 | 37.907 | 1.00 | 24.89 | O |
| HETATM | 3066 | O | HOH | E | 35 | −60.532 | 49.108 | 7.543 | 1.00 | 19.17 | O |
| HETATM | 3067 | O | HOH | E | 36 | −83.185 | 75.351 | 27.276 | 1.00 | 14.18 | O |
| HETATM | 3068 | O | HOH | E | 37 | −75.204 | 78.099 | 25.779 | 1.00 | 9.45 | O |
| HETATM | 3069 | O | HOH | E | 38 | −89.568 | 46.228 | 20.459 | 1.00 | 13.58 | O |
| HETATM | 3070 | O | HOH | E | 39 | −75.437 | 34.524 | −3.381 | 1.00 | 21.51 | O |
| HETATM | 3071 | O | HOH | E | 40 | −77.183 | 76.135 | 29.160 | 1.00 | 17.88 | O |
| HETATM | 3072 | O | HOH | E | 41 | −69.758 | −42.192 | 36.078 | 1.00 | 34.41 | O |
| HETATM | 3073 | O | HOH | E | 42 | −59.429 | 55.003 | 13.479 | 1.00 | 32.87 | O |
| HETATM | 3074 | O | HOH | E | 43 | −80.545 | 40.087 | 35.633 | 1.00 | 37.09 | O |
| HETATM | 3075 | O | HOH | E | 44 | −76.668 | 73.909 | 38.570 | 1.00 | 11.44 | O |
| HETATM | 3076 | O | HOH | E | 45 | −72.828 | 31.897 | −3.888 | 1.00 | 25.86 | O |
| HETATM | 3077 | O | HOH | E | 46 | −103.162 | 62.293 | 22.405 | 1.00 | 26.51 | O |
| HETATM | 3078 | O | HOH | E | 47 | −103.595 | 59.466 | 41.972 | 1.00 | 31.02 | O |
| HETATM | 3079 | O | HOH | E | 48 | −101.449 | 57.927 | 46.115 | 1.00 | 19.43 | O |
| HETATM | 3080 | O | HOH | E | 49 | −85.865 | 42.610 | 37.312 | 1.00 | 18.95 | O |
| HETATM | 3081 | O | HOH | E | 50 | −59.724 | 40.755 | −10.579 | 1.00 | 17.06 | O |
| HETATM | 3082 | O | HOH | E | 51 | −92.194 | 55.758 | 40.528 | 1.00 | 25.83 | O |
| HETATM | 3083 | O | HOH | E | 52 | −57.133 | 24.809 | 18.819 | 1.00 | 41.84 | O |
| HETATM | 3084 | O | HOH | E | 53 | −55.375 | 35.266 | −10.246 | 1.00 | 14.78 | O |
| HETATM | 3085 | O | HOH | E | 54 | −88.879 | 70.749 | 26.071 | 1.00 | 23.03 | O |
| HETATM | 3086 | O | HOH | E | 55 | −90.038 | 65.133 | 26.574 | 1.00 | 23.98 | O |
| HETATM | 3087 | O | HOH | E | 56 | −80.238 | 48.750 | 26.041 | 1.00 | 12.80 | O |
| HETATM | 3086 | O | HOH | E | 57 | −75.448 | −42.869 | 41.665 | 1.00 | 26.00 | O |
| HETATM | 3089 | O | HOH | E | 58 | −61.169 | 53.745 | −8.955 | 1.00 | 24.13 | O |
| HETATM | 3090 | O | HOH | E | 59 | −66.459 | 54.237 | 6.414 | 1.00 | 17.57 | O |
| HETATM | 3091 | O | HOH | E | 60 | −52.960 | 52.967 | 11.227 | 1.00 | 41.76 | O |
| HETATM | 3092 | O | HOH | E | 61 | −73.584 | 57.698 | 3.512 | 1.00 | 41.69 | O |
| HETATM | 3093 | O | HOH | E | 62 | −87.290 | 54.662 | 40.828 | 1.00 | 15.26 | O |
| HETATM | 3094 | O | HOH | E | 63 | −56.580 | 48.326 | 0.516 | 1.00 | 11.44 | O |
| HETATM | 3095 | O | HOH | E | 64 | −83.204 | 49.804 | 20.747 | 1.00 | 19.08 | O |
| HETATM | 3096 | O | HOH | E | 65 | −83.706 | 50.229 | 25.010 | 1.00 | 17.65 | O |
| HETATM | 3097 | O | HOH | E | 66 | −63.803 | 43.276 | −4.318 | 1.00 | 14.92 | O |
| HETATM | 3098 | O | HOH | E | 67 | −103.243 | 58.158 | 28.487 | 1.00 | 15.09 | O |
| HETATM | 3099 | O | HOH | E | 68 | −83.498 | 48.158 | 29.169 | 1.00 | 10.28 | O |
| HETATM | 3100 | O | HOH | E | 69 | −89.281 | 64.788 | 40.375 | 1.00 | 15.60 | O |
| HETATM | 3101 | O | HOH | E | 70 | −84.688 | 49.155 | 41.191 | 1.00 | 15.52 | O |
| HETATM | 3102 | O | HOH | E | 71 | −79.871 | 41.592 | 27.292 | 1.00 | 22.67 | O |
| HETATM | 3103 | O | HOH | E | 72 | −69.490 | 49.878 | 14.907 | 1.00 | 30.11 | O |
| HETATM | 3104 | O | HOH | E | 73 | −73.559 | 46.387 | 5.062 | 1.00 | 20.73 | O |
| HETATM | 3105 | O | HOH | E | 74 | −82.498 | 52.140 | 17.585 | 1.00 | 17.79 | O |
| HETATM | 3106 | O | HOH | E | 75 | −53.788 | 24.248 | 3.591 | 1.00 | 21.90 | O |
| HETATM | 3107 | O | HOH | E | 76 | −77.750 | 59.889 | −2.726 | 1.00 | 41.87 | O |
| HETATM | 3108 | O | HOH | E | 77 | −50.934 | 22.073 | 12.093 | 1.00 | 20.04 | O |
| HETATM | 3109 | O | HOH | E | 78 | −68.606 | 53.008 | 21.278 | 1.00 | 28.58 | O |
| HETATM | 3110 | O | HOH | E | 79 | −86.292 | 41.620 | 34.804 | 1.00 | 14.10 | O |
| HETATM | 3111 | O | HOH | E | 80 | −81.480 | 66.767 | 38.024 | 1.00 | 25.04 | O |
| HETATM | 3112 | O | HOH | E | 81 | −76.688 | 59.038 | 41.746 | 1.00 | 29.14 | O |
| HETATM | 3113 | O | HOH | E | 82 | −82.777 | 63.346 | 42.410 | 1.00 | 25.00 | O |
| HETATM | 3114 | O | HOH | E | 83 | −84.018 | 72.114 | 32.905 | 1.00 | 28.85 | O |
| HETATM | 3115 | O | HOH | E | 84 | −95.459 | 61.386 | 44.347 | 1.00 | 19.99 | O |
| HETATM | 3116 | O | HOH | E | 85 | −99.973 | 63.295 | 38.023 | 1.00 | 15.69 | O |
| HETATM | 3117 | O | HOH | E | 86 | −103.069 | 53.884 | 39.776 | 1.00 | 21.47 | O |
| HETATM | 3118 | O | HOH | E | 87 | −99.974 | 49.841 | 38.988 | 1.00 | 26.64 | O |
| HETATM | 3119 | O | HOH | E | 88 | −93.272 | 44.134 | 38.296 | 1.00 | 17.33 | O |
| HETATM | 3120 | O | HOH | E | 89 | −84.497 | 47.074 | 43.167 | 1.00 | 31.47 | O |
| HETATM | 3121 | O | HOH | E | 90 | −95.616 | 58.048 | 43.705 | 1.00 | 24.24 | O |
| HETATM | 3122 | O | HOH | E | 91 | −93.058 | 53.308 | 20.984 | 1.00 | 20.72 | O |
| HETATM | 3123 | O | HOH | E | 92 | −64.726 | 25.612 | −4.759 | 1.00 | 19.02 | O |
| HETATM | 3124 | O | HOH | E | 93 | −64.080 | 54.392 | 7.733 | 1.00 | 25.88 | O |
| HETATM | 3125 | O | HOH | E | 94 | −75.414 | 46.688 | 14.586 | 1.00 | 23.70 | O |
| HETATM | 3126 | O | HOH | E | 95 | −76.919 | 60.434 | 22.878 | 1.00 | 29.02 | O |
| HETATM | 3127 | O | HOH | E | 96 | −72.121 | 51.684 | −9.181 | 1.00 | 22.07 | O |
| HETATM | 3128 | O | HOH | E | 97 | −69.801 | 29.308 | −6.777 | 1.00 | 29.73 | O |
| HETATM | 3129 | O | HOH | E | 98 | −67.793 | 52.700 | 27.214 | 1.00 | 19.63 | O |
| HETATM | 3130 | O | HOH | E | 99 | −65.229 | 47.087 | 31.188 | 1.00 | 20.46 | O |
| HETATM | 3131 | O | HOH | E | 100 | −71.780 | 36.479 | −15.284 | 1.00 | 20.89 | O |
| HETATM | 3132 | O | HOH | E | 101 | −66.175 | 17.583 | 11.874 | 1.00 | 17.74 | O |
| HETATM | 3133 | O | HOH | E | 102 | −72.897 | 49.688 | 21.236 | 1.00 | 17.64 | O |
| HETATM | 3134 | O | HOH | E | 103 | −97.840 | 62.433 | 25.902 | 1.00 | 24.04 | O |
| HETATM | 3135 | O | HOH | E | 104 | −80.929 | 42.036 | 22.884 | 1.00 | 17.19 | O |
| HETATM | 3136 | O | HOH | E | 105 | −87.861 | 56.034 | 43.071 | 1.00 | 19.39 | O |
| HETATM | 3137 | O | HOH | E | 106 | −67.652 | 29.224 | −13.993 | 1.00 | 29.59 | O |
| HETATM | 3138 | O | HOH | E | 107 | −67.029 | 51.476 | 12.895 | 1.00 | 26.62 | O |
| HETATM | 3139 | O | HOH | E | 108 | −62.685 | 44.719 | 19.793 | 1.00 | 27.42 | O |
| HETATM | 3140 | O | HOH | E | 109 | −58.392 | 45.582 | −15.053 | 1.00 | 39.55 | O |
| HETATM | 3141 | O | HOH | E | 110 | −68.600 | 57.559 | 11.613 | 1.00 | 29.66 | O |
| HETATM | 3142 | O | HOH | E | 111 | −71.053 | 57.875 | 9.205 | 1.00 | 28.87 | O |
| HETATM | 3143 | O | HOH | E | 112 | −58.345 | 30.273 | −17.991 | 1.00 | 36.85 | O |
| HETATM | 3144 | O | HOH | E | 113 | −62.076 | 31.591 | −14.780 | 1.00 | 35.18 | O |
| HETATM | 3145 | O | HOH | E | 114 | −69.515 | 45.588 | 14.301 | 1.00 | 25.81 | O |
| HETATM | 3146 | O | HOH | E | 115 | −67.400 | 42.679 | 12.960 | 1.00 | 30.03 | O |
| HETATM | 3147 | O | HOH | E | 116 | −54.107 | 19.763 | 13.350 | 1.00 | 27.18 | O |
| HETATM | 3148 | O | HOH | E | 117 | −71.926 | 77.526 | 37.394 | 1.00 | 16.72 | O |
| HETATM | 3149 | O | HOH | E | 118 | −70.190 | 75.537 | 37.394 | 1.00 | 23.42 | O |
| HETATM | 3150 | O | HOH | E | 119 | −70.548 | 74.002 | 40.182 | 1.00 | 29.73 | O |
| HETATM | 3151 | O | HOH | E | 120 | −54.175 | 46.140 | −9.850 | 1.00 | 21.67 | O |
| HETATM | 3152 | O | HOH | E | 121 | −58.883 | 46.277 | −12.947 | 1.00 | 30.55 | O |
| HETATM | 3153 | O | HOH | E | 122 | −70.371 | 43.756 | 24.324 | 1.00 | 24.68 | O |
| HETATM | 3154 | O | HOH | E | 123 | −83.507 | 54.035 | 16.237 | 1.00 | 59.51 | O |
| HETATM | 3155 | O | HOH | E | 124 | −52.052 | 42.103 | 5.232 | 1.00 | 25.40 | O |
| HETATM | 3156 | O | HOH | E | 125 | −54.196 | 31.640 | −3.139 | 1.00 | 27.56 | O |
| HETATM | 3157 | O | HOH | E | 126 | −69.781 | 58.858 | 0.675 | 1.00 | 23.56 | O |
| HETATM | 3158 | O | HOH | E | 127 | −57.856 | 51.065 | 11.860 | 1.00 | 30.34 | O |
| HETATM | 3159 | O | HOH | E | 128 | −55.012 | 46.601 | −1.743 | 1.00 | 17.94 | O |
| HETATM | 3160 | O | HOH | E | 129 | −76.148 | 42.728 | −3.456 | 1.00 | 24.18 | O |
| HETATM | 3161 | O | HOH | E | 130 | −73.919 | 43.481 | −2.116 | 1.00 | 22.90 | O |
| HETATM | 3162 | O | HOH | E | 131 | −76.043 | 49.279 | −0.387 | 1.00 | 21.18 | O |
| HETATM | 3163 | O | HOH | E | 132 | −71.128 | 50.114 | −0.487 | 1.00 | 29.14 | O |
| HETATM | 3164 | O | HOH | E | 133 | −69.676 | 59.336 | 24.913 | 1.00 | 39.28 | O |
| HETATM | 3165 | O | HOH | E | 134 | −69.959 | 59.928 | 27.554 | 1.00 | 17.29 | O |
| HETATM | 3166 | O | HOH | E | 135 | −70.066 | 57.577 | 28.592 | 1.00 | 26.78 | O |
| HETATM | 3167 | O | HOH | E | 136 | −70.365 | 59.873 | 33.572 | 1.00 | 22.20 | O |
| HETATM | 3168 | O | HOH | E | 137 | −70.924 | 52.296 | 16.259 | 1.00 | 23.64 | O |
| HETATM | 3169 | O | HOH | E | 138 | −68.059 | 49.481 | 36.909 | 1.00 | 28.99 | O |
| HETATM | 3170 | O | HOH | E | 139 | −92.425 | 45.870 | 24.462 | 1.00 | 32.22 | O |
| HETATM | 3171 | O | HOH | E | 140 | −70.593 | 73.375 | 34.037 | 1.00 | 19.44 | O |
| HETATM | 3172 | O | HOH | E | 141 | −70.381 | 68.367 | 35.515 | 1.00 | 29.60 | O |
| HETATM | 3173 | O | HOH | E | 142 | −70.832 | 77.231 | 26.320 | 1.00 | 26.91 | O |
| HETATM | 3174 | O | HOH | E | 143 | −82.839 | 69.582 | 34.960 | 1.00 | 18.99 | O |
| HETATM | 3175 | O | HOH | E | 144 | −102.602 | 60.488 | 44.676 | 1.00 | 27.35 | O |
| HETATM | 3176 | O | HOH | E | 145 | −79.427 | 50.334 | 11.103 | 1.00 | 55.01 | O |
| HETATM | 3177 | O | HOH | E | 146 | −63.396 | 45.954 | 14.353 | 1.00 | 47.29 | O |
| HETATM | 3178 | O | HOH | E | 147 | −52.773 | 55.530 | 10.280 | 1.00 | 49.57 | O |
| HETATM | 3179 | O | HOH | E | 148 | −89.693 | 48.863 | 39.532 | 1.00 | 51.10 | O |
| HETATM | 3180 | O | HOH | E | 149 | −104.729 | 65.257 | 26.309 | 1.00 | 33.94 | O |
| HETATM | 3181 | O | HOH | E | 150 | −65.355 | 47.093 | 2.538 | 1.00 | 27.90 | O |
| HETATM | 3182 | O | HOH | E | 151 | −79.622 | 78.600 | 14.175 | 1.00 | 32.96 | O |
| HETATM | 3183 | O | HOH | E | 152 | −92.653 | 57.258 | 14.907 | 1.00 | 21.40 | O |
| HETATM | 3184 | O | HOH | E | 153 | −68.813 | 49.274 | −11.365 | 1.00 | 19.58 | O |
| HETATM | 3185 | O | HOH | E | 154 | −68.354 | 52.897 | 23.733 | 1.00 | 39.44 | O |
| HETATM | 3186 | O | HOH | E | 155 | −61_714 | 29.744 | 18.342 | 1.00 | 30.53 | O |
| HETATM | 3187 | O | HOH | E | 156 | −88.957 | 56.165 | 15.691 | 1.00 | 30.53 | O |
| HETATM | 3188 | O | HOH | E | 157 | −70.218 | 30.473 | −2.090 | 1.00 | 17.06 | O |
| HETATM | 3189 | O | HOH | E | 158 | −72.024 | 25.624 | −9.927 | 1.00 | 35.41 | O |
| HETATM | 3190 | O | HOH | E | 159 | −103.811 | 48.879 | 34.220 | 1.00 | 28.68 | O |
| HETATM | 3191 | O | HOH | E | 160 | −84.794 | 45.802 | 14.082 | 1.00 | 43.97 | O |
| HETATM | 3192 | O | HOH | E | 161 | −99.058 | 61.581 | 12./152 | 1.00 | 33.00 | O |
| HETATM | 3193 | O | HOH | E | 162 | −72.897 | 45.361 | −6.669 | 1.00 | 25.13 | O |
| HETATM | 3194 | O | HOH | E | 163 | −78.162 | 30.405 | 9.747 | 1.00 | 28.31 | O |
| HETATM | 3195 | O | HOH | E | 164 | −81.159 | 38.504 | −8.512 | 1.00 | 24.93 | O |
| HETATM | 3196 | O | HOH | E | 165 | −70.363 | 70.455 | 33.678 | 1.00 | 33.26 | O |
| HETATM | 3197 | O | HOH | E | 166 | −65.978 | 47.375 | 36.006 | 1.00 | 27.95 | O |
| HETATM | 3198 | O | HOH | E | 167 | −63.904 | 72.823 | 29.870 | 1.00 | 31.88 | O |

END

APPENDIX B
Atomic Coordinates of FIG. 14 Right Panel (in vitro formed PANDA)

| HEADER | ———— | 11-AUG-17 xxxx | | | | | |
|---|---|---|---|---|---|---|---|
| COMPND | ——— | | | | | | |
| REMARK | 3 | | | | | | |
| REMARK | 3 | REFINEMENT. | | | | | |
| REMARK | 3 | PROGRAM : REEMAC 5.8.0158 | | | | | |
| REMARK | 3 | AUTHORS : MURSHUDOV,SKUBAK,LEBEDEV,PANNU, | | | | | |
| REMARK | 3 | STEINER,NICHOLLS,WINN,LONG,VAGIN | | | | | |
| REMARK | 3 | | | | | | |
| REMARK | 3 | REFINEMENT TARGET : MAXIMUM LIKELIHOOD | | | | | |
| REMARK | 3 | | | | | | |
| REMARK | 3 | DATA USED IN REFINEMENT. | | | | | |
| REMARK | 3 | RESOLUTION RANGE HIGH (ANGSTROMS) : 2.30 | | | | | |
| REMARK | 3 | RESOLUTION RANGE LOW (ANGSTROMS) : 84.16 | | | | | |
| REMARK | 3 | DATA CUTOFF (SIGMA(F)) : NONE | | | | | |
| REMARK | 3 | COMPLETENESS FOR RANGE (%) : 97.43 | | | | | |
| REMARK | 3 | NUMBER OF REFLECTIONS : 32384 | | | | | |
| REMARK | 3 | | | | | | |
| REMARK | 3 | FIT TO DATA USED IN REFINEMENT. | | | | | |
| REMARK | 3 | CROSS-VALIDATION METHOD : THROUGHOUT | | | | | |
| REMARK | 3 | FREE R VALUE TEST SET SELECTION : RANDOM | | | | | |
| REMARK | 3 | R VALUE (WORKING + TEST SET) : 0.25722 | | | | | |
| REMARK | 3 | R VALUE (WORKING SET) : 0.25549 | | | | | |
| REMARK | 3 | FREE R VALUE : 0.29192 | | | | | |
| REMARK | 3 | FREE R VALUE TEST SET SIZE (%) : 5.0 | | | | | |
| REMARK | 3 | FREE R VALUE TEST SET COUNT : 1708 | | | | | |
| REMARK | 3 | | | | | | |
| REMARK | 3 | FIT IN THE HIGHEST RESOLUTION BIN. | | | | | |
| REMARK | 3 | TOTAL NUMBER OF BINS USED : 20 | | | | | |
| REMARK | 3 | BIN RESOLUTION RANGE HIGH : 2.300 | | | | | |
| REMARK | 3 | BIN RESOLUTION RANGE LOW : 2.360 | | | | | |
| REMARK | 3 | REFLECTION IN BIN (WORKING SET) : 2468 | | | | | |
| REMARK | 3 | BIN COMPLETENESS (WORKING + TEST) (%) : 98.88 | | | | | |
| REMARK | 3 | BIN R VALUE (WORKING SET) : 0.352 | | | | | |
| REMARK | 3 | BIN FREE R VALUE SET COUNT : 82 | | | | | |
| REMARK | 3 | BIN FREE R VALUE : 0.336 | | | | | |
| REMARK | 3 | | | | | | |
| REMARK | 3 | NUMBER OF NON-HYDROGEN ATOMS USED IN REFINEMENT. | | | | | |
| REMARK | 3 | ALL ATOMS : 6024 | | | | | |
| REMARK | 3 | | | | | | |
| REMARK | 3 | B VALUES. | | | | | |
| REMARK | 3 | FROM WILSON PLOT (A**2) : NULL | | | | | |
| REMARK | 3 | MEAN B VALUE (OVERALL, A**2) : 34.234 | | | | | |
| REMARK | 3 | OVERALL ANISOTROPIC B VALUE. | | | | | |
| REMARK | 3 | B11 (A**2) : −2.83 | | | | | |
| REMARK | 3 | B22 (A**2) : 4.92 | | | | | |
| REMARK | 3 | B33 (A**2) : −2.10 | | | | | |
| REMARK | 3 | B12 (A**2) : −0.00 | | | | | |
| REMARK | 3 | B13 (A**2) : 1.22 | | | | | |
| REMARK | 3 | B23 (A**2) : 0.00 | | | | | |
| REMARK | 3 | | | | | | |
| REMARK | 3 | ESTIMATED OVERALL COORDINATE ERROR. | | | | | |
| REMARK | 3 | ESU BASED ON R VALUE (A) : 0.514 | | | | | |
| REMARK | 3 | ESU BASED ON FREE R VALUE (A) : 0.297 | | | | | |
| REMARK | 3 | ESU BASED ON MAXIMUM LIKELIHOOD (A) : 0.255 | | | | | |
| REMARK | 3 | ESU FOR B VALUES BASED ON MAXIMUM LIKELIHOOD (A**2) : 10.679 | | | | | |
| REMARK | 3 | | | | | | |
| REMARK | 3 | CORRELATION COEFFICIENTS. | | | | | |
| REMARK | 3 | CORRELATION COEFFICIENT FO-FC : 0.893 | | | | | |
| REMARK | 3 | CORRELATION COEFFICIENT FO-FC FREE : 0.856 | | | | | |
| REMARK | 3 | | | | | | |
| REMARK | 3 | RMS DEVIATIONS FROM IDEAL VALUES | COUNT | | RMS | | WEIGHT |
| REMARK | 3 | BOND LENGTHS REFINED ATOMS (A) : | 5975 | ; | 0.009 | ; | 0.019 |
| REMARK | 3 | BOND LENGTHS OTHERS (A) : | 5381 | ; | 0.002 | ; | 0.020 |
| REMARK | 3 | BOND ANGLES REFINED ATOMS (DEGREES) : | 8097 | ; | 1.346 | ; | 1.962 |
| REMARK | 3 | BOND ANGLES OTHERS (DEGREES) : | 12500 | ; | 0.933 | ; | 3.002 |
| REMARK | 3 | TORSION ANGLES, PERIOD 1 (DEGREES) : | 735 | ; | 5.871 | ; | 5.000 |
| REMARK | 3 | TORSION ANGLES, PERIOD 2 (DEGREES) : | 274 | ; | 30.963 | ; | 22.555 |
| REMARK | 3 | TORSION ANGLES, PERIOD 3 (DEGREES) : | 986 | ; | 13.454 | ; | 15.000 |
| REMARK | 3 | TORSION ANGLES, PERIOD 4 (DEGREES) : | 62 | ; | 14.606 | ; | 15.000 |
| REMARK | 3 | CHIRAL-CENTER RESTRAINTS (A**3) : | 889 | ; | 0.073 | ; | 0.200 |
| REMARK | 3 | GENERAL PLANES REFINED ATOMS (A) : | 6619 | ; | 0.006 | ; | 0.021 |
| REMARK | 3 | GENERAL PLANES OTHERS (A) : | 1227 | ; | 0.001 | ; | 0.020 |
| REMARK | 3 | | | | | | |
| REMARK | 3 | ISOTROPIC THERMAL FACTOR RESTRAINTS. | COUNT | | RMS | | WEIGHT |
| REMARK | 3 | MAIN-CHAIN BOND REFINED ATOMS (A**2) : | 2961 | ; | 1.571 | ; | 3.444 |
| REMARK | 3 | MAIN-CHAIN BOND OTHER ATOMS (A**2) : | 2960 | ; | 1.571 | ; | 3.443 |
| REMARK | 3 | MAIN-CHAIN ANGLE REFINED ATOMS (A**2) : | 3689 | ; | 2.815 | ; | 5.141 |
| REMARK | 3 | MAIN-CHAIN ANGLE OTHER ATOMS (A**2) : | 3690 | ; | 2.814 | ; | 5.142 |
| REMARK | 3 | SIDE-CHAIN BOND REFINED ATOMS (A**2) : | 3014 | ; | 1.417 | ; | 3.600 |
| REMARK | 3 | SIDE-CHAIN BOND OTHER ATOMS (A**2) : | 3014 | ; | 1.417 | ; | 3.600 |
| REMARK | 3 | SIDE-CHAIN ANGLE OTHER ATOMS (A**2) : | 4409 | ; | 2.302 | ; | 5.324 |
| REMARK | 3 | LONG RANGE B REFINED ATOMS (A**2) : | 6103 | ; | 4.855 | ; | 39.321 |
| REMARK | 3 | LONG RANGE B OTHER ATOMS (A**2) : | 6086 | ; | 4.853 | ; | 39.339 |

| REMARK | 3 | | | | | | | | | | |
|---|---|---|---|---|---|---|---|---|---|---|---|
| REMARK | 3 | NCS RESTRAINTS STATISTICS | | | | | | | | | |
| REMARK | 3 | NCS TYPE: LOCAL | | | | | | | | | |
| REMARK | 3 | NUMBER OF DIFFERENT NCS PAIRS : 6 | | | | | | | | | |
| REMARK | 3 | GROUP | CHAIN1 | | RANGE | CHAIN2 | | RANGE | COUNT | RMS | WEIGHT |
| REMARK | 3 | 1 | A | 96 | 287 | B | 96 | 287 | 11606 | 0.06 | 0.05 |
| REMARK | 3 | 2 | A | 97 | 290 | C | 97 | 290 | 10180 | 0.08 | 0.05 |
| REMARK | 3 | 3 | A | 96 | 288 | D | 96 | 288 | 10296 | 0.08 | 0.05 |
| REMARK | 3 | 4 | B | 97 | 288 | C | 97 | 288 | 10034 | 0.08 | 0.05 |
| REMARK | 3 | 5 | B | 96 | 288 | D | 96 | 288 | 10236 | 0.08 | 0.05 |
| REMARK | 3 | 6 | C | 97 | 287 | D | 97 | 287 | 9974 | 0.07 | 0.05 |

| REMARK | 3 | | | | | | | | | | | |
|---|---|---|---|---|---|---|---|---|---|---|---|---|
| REMARK | 3 | TWIN DETAILS | | | | | | | | | | |
| REMARK | 3 | NUMBER OF TWIN DOMAINS : NULL | | | | | | | | | | |
| REMARK | 3 | | | | | | | | | | | |
| REMARK | 3 | | | | | | | | | | | |
| REMARK | 3 | TLS DETAILS | | | | | | | | | | |
| REMARK | 3 | NUMBER OF TLS GROUPS : NULL | | | | | | | | | | |
| REMARK | 3 | | | | | | | | | | | |
| REMARK | 3 | | | | | | | | | | | |
| REMARK | 3 | BULK SOLVENT MODELLING. | | | | | | | | | | |
| REMARK | 3 | METHOD USED : MASK | | | | | | | | | | |
| REMARK | 3 | PARAMETERS FOR MASK CALCULATION | | | | | | | | | | |
| REMARK | 3 | VDW PROBE RADIUS : 1.20 | | | | | | | | | | |
| REMARK | 3 | ION PROBE RADIUS : 0.80 | | | | | | | | | | |
| REMARK | 3 | SHRINKAGE RADIUS : 0.80 | | | | | | | | | | |
| REMARK | 3 | | | | | | | | | | | |
| REMARK | 3 | OTHER REFINEMENT REMARKS: | | | | | | | | | | |
| REMARK | 3 | HYDROGENS HAVE BEEN ADDED IN THE RIDING POSITIONS | | | | | | | | | | |
| REMARK | 3 | U VALUES : REFINED INDIVIDUALLY | | | | | | | | | | |
| REMARK | 3 | | | | | | | | | | | |
| LINK | | AS | ARS | F | 1 | SG | CYS | C | 141 | 1555 | 1555 | 2.14 |
| LINK | | AS | ARS | F | 1 | SG | CYS | C | 124 | 1555 | 1555 | 2.14 |
| LINK | | AS | ARS | F | 1 | SG | CYS | C | 135 | 1555 | 1555 | 2.14 |
| LINK | | AS | ARS | F | 2 | SG | CYS | D | 135 | 1555 | 1555 | 2.14 |
| LINK | | AS | ARS | F | 2 | SG | CYS | D | 141 | 1555 | 1555 | 2.14 |
| LINK | | AS | ARS | F | 2 | SG | CYS | D | 124 | 1555 | 1555 | 2.14 |
| LINKR | | ZN | ZN | E | 2 | SG | CYS | B | 176 | | | ZN-CYS |
| LINKR | | ZN | ZN | E | 2 | SG | CYS | B | 242 | | | ZN-CYS |
| LINKR | | ZN | ZN | E | 2 | SG | CYS | B | 238 | | | ZN-CYS |
| LINKR | | ZN | ZN | E | 2 | ND1 | HIS | B | 179 | | | ZN-HISND |
| LINKR | | ZN | ZN | E | 1 | SG | CYS | A | 238 | | | ZN-CYS |
| LINKR | | ZN | ZN | E | 1 | SG | CYS | A | 242 | | | ZN-CYS |
| LINKR | | ZN | ZN | E | 1 | SG | CYS | A | 176 | | | ZN-CYS |
| LINKR | | ZN | ZN | E | 1 | ND1 | HIS | A | 179 | | | ZN-HISND |
| LINKR | | | PRO | C | 177 | | ALA | C | 189 | | | gap |
| LINKR | | | SER | C | 241 | | PRO | C | 250 | | | gap |
| LINKR | | | PRO | D | 177 | | LEU | D | 188 | | | gap |
| LINKR | | | SER | D | 241 | | SER | D | 249 | | | gap |

| | | | | | | | | | | |
|---|---|---|---|---|---|---|---|---|---|---|
| CRYST1 | 66.673 | 68.634 | 84.163 | 90.00 | 90.25 | 90.00 | P | 1 | 21 | 1 |

| | | | | |
|---|---|---|---|---|
| SCALE1 | 0.014562 | 0.000000 | 0.000064 | 0.00000 |
| SCALE2 | −0.000000 | 0.014570 | 0.000000 | 0.00000 |
| SCALE3 | 0.000000 | −0.000000 | 0.011882 | 0.00000 |

| | | | | | | | | | | | |
|---|---|---|---|---|---|---|---|---|---|---|---|
| ATOM | 1 | N | SER | A | 95 | −14.627 | 23.029 | −33.776 | 1.00 | 55.61 | N |
| ATOM | 2 | CA | SER | A | 95 | −13.173 | 23.286 | −34.047 | 1.00 | 55.20 | C |
| ATOM | 3 | CB | SER | A | 95 | −12.945 | 24.782 | −34.321 | 1.00 | 55.70 | C |
| ATOM | 4 | OG | SER | A | 95 | −13.321 | 25.567 | −33.199 | 1.00 | 55.62 | O |
| ATOM | 5 | C | SER | A | 95 | −12.267 | 22.802 | −32.899 | 1.00 | 53.35 | C |
| ATOM | 6 | O | SER | A | 95 | −11.213 | 23.397 | −32.629 | 1.00 | 53.86 | O |
| ATOM | 7 | N | SER | A | 96 | −12.677 | 21.714 | −32.243 | 1.00 | 49.49 | N |
| ATOM | 8 | CA | SER | A | 96 | −11.939 | 21.141 | −31.109 | 1.00 | 46.38 | C |
| ATOM | 9 | CB | SER | A | 96 | −12.902 | 20.396 | −30.174 | 1.00 | 47.46 | C |
| ATOM | 10 | OG | SER | A | 96 | —12.226 | 19.755 | −29.093 | 1.00 | 50.16 | O |
| ATOM | 11 | C | SER | A | 96 | −10.829 | 20.192 | −31.577 | 1.00 | 42.79 | C |
| ATOM | 12 | O | SER | A | 96 | −10.994 | 19.455 | −32.560 | 1.00 | 42.24 | O |
| ATOM | 13 | N | VAL | A | 97 | −9.709 | 20.220 | −30.852 | 1.00 | 38.31 | N |
| ATOM | 14 | CA | VAL | A | 97 | −8.560 | 19.351 | −31.085 | 1.00 | 35.88 | C |
| ATOM | 15 | CB | VAL | A | 97 | −7.346 | 20.175 | −31.554 | 1.00 | 36.79 | C |
| ATOM | 16 | CG1 | VAL | A | 97 | −6.084 | 19.324 | −31.699 | 1.00 | 38.20 | C |
| ATOM | 17 | CG2 | VAL | A | 97 | −7.676 | 20.864 | −32.886 | 1.00 | 37.51 | C |
| ATOM | 18 | C | VAL | A | 97 | −6.201 | 18.624 | −29.777 | 1.00 | 33.29 | C |
| ATOM | 19 | O | VAL | A | 97 | −7.883 | 19.280 | −28.799 | 1.00 | 30.83 | O |
| ATOM | 20 | N | PRO | A | 98 | −8.226 | 17.264 | −29.775 | 1.00 | 30.57 | N |
| ATOM | 21 | CA | PRO | A | 98 | −7.799 | 16.523 | −28.592 | 1.00 | 30.15 | C |
| ATOM | 22 | CB | PRO | A | 98 | −8.007 | 15.045 | −28.970 | 1.00 | 30.13 | C |
| ATOM | 23 | CG | PRO | A | 98 | −8.810 | 15.051 | −30.196 | 1.00 | 30.80 | C |
| ATOM | 24 | CD | PRO | A | 98 | −8.697 | 16.372 | −30.847 | 1.00 | 30.89 | C |
| ATOM | 25 | C | PRO | A | 98 | −6.332 | 16.757 | −28.271 | 1.00 | 29.77 | C |
| ATOM | 26 | O | PRO | A | 98 | −5.523 | 16.897 | −29.185 | 1.00 | 29.28 | O |
| ATOM | 27 | N | SER | A | 99 | −6.005 | 16.833 | −26.981 | 1.00 | 28.87 | N |
| ATOM | 28 | CA | SER | A | 99 | −4.642 | 17.120 | −26.559 | 1.00 | 29.21 | C |
| ATOM | 29 | CB | SER | A | 99 | −4.577 | 17.353 | −25.042 | 1.00 | 30.19 | C |
| ATOM | 30 | OG | SER | A | 99 | −3.231 | 17.378 | −24.590 | 1.00 | 31.00 | O |
| ATOM | 31 | C | SER | A | 99 | −3.710 | 15.976 | −26.930 | 1.00 | 29.17 | C |
| ATOM | 32 | O | SER | A | 99 | −4.093 | 14.806 | −26.867 | 1.00 | 28.26 | O |
| ATOM | 33 | N | GLN | A | 100 | −2.490 | 16.332 | −27.314 | 1.00 | 29.67 | N |
| ATOM | 34 | CA | GLN | A | 100 | −1.431 | 15.361 | −27.605 | 1.00 | 30.31 | C |
| ATOM | 35 | CB | GLN | A | 100 | −1.003 | 15.483 | −29.067 | 1.00 | 30.08 | C |
| ATOM | 36 | CG | GLN | A | 100 | −0.276 | 16.765 | −29.442 | 1.00 | 29.96 | C |
| ATOM | 37 | CD | GLN | A | 100 | 0.184 | 16.759 | −30.884 | 1.00 | 30.51 | C |
| ATOM | 38 | OE1 | GLN | A | 100 | −0.622 | 16.833 | −31.823 | 1.00 | 31.13 | O |
| ATOM | 39 | NE2 | GLN | A | 100 | 1.497 | 16.691 | −31.076 | 1.00 | 30.81 | N |
| ATOM | 40 | C | GLN | A | 100 | −0.218 | 15.489 | −26.670 | 1.00 | 30.76 | C |
| ATOM | 41 | O | GLN | A | 100 | 0.825 | 14.894 | −26.934 | 1.00 | 29.98 | O |
| ATOM | 42 | N | LYS | A | 101 | −0.363 | 16.249 | −25.583 | 1.00 | 32.59 | N |
| ATOM | 43 | CA | LYS | A | 101 | 0.737 | 16.494 | −24.653 | 1.00 | 33.70 | C |
| ATOM | 44 | CB | LYS | A | 101 | 0.366 | 17.593 | −23.646 | 1.00 | 35.98 | C |
| ATOM | 45 | CG | LYS | A | 101 | 1.421 | 17.937 | −22.587 | 1.00 | 39.59 | C |
| ATOM | 46 | CD | LYS | A | 101 | 2.733 | 18.430 | −23.173 | 1.00 | 42.11 | C |
| ATOM | 47 | CE | LYS | A | 101 | 3.731 | 18.742 | −22.055 | 1.00 | 44.20 | C |
| ATOM | 48 | NZ | LYS | A | 101 | 3.382 | 19.987 | −21.309 | 1.00 | 45.71 | N |
| ATOM | 49 | C | LYS | A | 101 | 1.086 | 15.198 | −23.922 | 1.00 | 31.90 | C |
| ATOM | 50 | O | LYS | A | 101 | 0.214 | 14.551 | −23.352 | 1.00 | 28.77 | O |
| ATOM | 51 | N | THR | A | 102 | 2.365 | 14.830 | −23.966 | 1.00 | 31.39 | N |
| ATOM | 52 | CA | THR | A | 102 | 2.836 | 13.624 | −23.298 | 1.00 | 32.15 | C |
| ATOM | 53 | CB | THR | A | 102 | 4.303 | 13.337 | −23.651 | 1.00 | 32.13 | C |
| ATOM | 54 | OG1 | THR | A | 102 | 4.404 | 13.167 | −25.070 | 1.00 | 31.70 | O |
| ATOM | 55 | CG2 | THR | A | 102 | 4.814 | 12.070 | −22.960 | 1.00 | 32.37 | C |
| ATOM | 56 | C | THR | A | 102 | 2.674 | 13.762 | −21.782 | 1.00 | 32.61 | C |
| ATOM | 57 | O | THR | A | 102 | 2.882 | 14.832 | −21.209 | 1.00 | 32.02 | O |
| ATOM | 58 | N | TYR | A | 103 | 2.266 | 12.667 | −21.149 | 1.00 | 33.21 | N |
| ATOM | 59 | CA | TYR | A | 103 | 1.988 | 12.648 | −19.725 | 1.00 | 32.97 | C |
| ATOM | 60 | CB | TYR | A | 103 | 0.577 | 13.178 | −19.522 | 1.00 | 33.83 | C |
| ATOM | 61 | CG | TYR | A | 103 | 0.055 | 13.176 | −18.109 | 1.00 | 35.21 | C |
| ATOM | 62 | CD1 | TYR | A | 103 | 0.738 | 13.823 | −17.064 | 1.00 | 36.65 | C |
| ATOM | 63 | CE1 | TYR | A | 103 | 0.237 | 13.824 | −15.768 | 1.00 | 36.76 | C |
| ATOM | 64 | CZ | TYR | A | 103 | −0.941 | 13.122 | −15.486 | 1.00 | 37.59 | C |
| ATOM | 65 | OH | TYR | A | 103 | −1.442 | 13.094 | −14.204 | 1.00 | 38.34 | O |
| ATOM | 66 | CE2 | TYR | A | 103 | −1.611 | 12.455 | −16.495 | 1.00 | 36.31 | C |
| ATOM | 67 | CD2 | TYR | A | 103 | −1.089 | 12.484 | −17.801 | 1.00 | 35.90 | C |
| ATOM | 68 | C | TYR | A | 103 | 2.164 | 11.232 | −19.197 | 1.00 | 31.55 | C |
| ATOM | 69 | O | TYR | A | 103 | 1.341 | 10.370 | −19.486 | 1.00 | 30.86 | O |
| ATOM | 70 | N | GLN | A | 104 | 3.246 | 10.985 | −18.460 | 1.00 | 30.26 | N |
| ATOM | 71 | CA | GLN | A | 104 | 3.483 | 9.661 | −17.874 | 1.00 | 30.21 | C |
| ATOM | 72 | CB | GLN | A | 104 | 4.937 | 9.483 | −17.415 | 1.00 | 31.15 | C |
| ATOM | 73 | CG | GLN | A | 104 | 5.954 | 9.619 | −18.546 | 1.00 | 32.35 | C |
| ATOM | 74 | CD | GLN | A | 104 | 7.246 | 8.839 | −18.352 | 1.00 | 33.05 | C |
| ATOM | 75 | OE1 | GLN | A | 104 | 7.457 | 8.200 | −17.329 | 1.00 | 33.24 | O |
| ATOM | 76 | NE2 | GLN | A | 104 | 8.104 | 8.883 | −19.357 | 1.00 | 33.95 | N |
| ATOM | 77 | C | GLN | A | 104 | 2.524 | 9.334 | −16.736 | 1.00 | 29.46 | C |
| ATOM | 78 | O | GLN | A | 104 | 2.173 | 8.173 | −16.522 | 1.00 | 29.04 | O |
| ATOM | 79 | N | GLY | A | 105 | 2.122 | 10.356 | −15.998 | 1.00 | 29.02 | N |
| ATOM | 80 | CA | GLY | A | 105 | 1.157 | 10.218 | −14.903 | 1.00 | 29.56 | C |
| ATOM | 81 | C | GLY | A | 105 | 1.680 | 9.474 | −13.692 | 1.00 | 29.22 | C |
| ATOM | 82 | O | GLY | A | 105 | 2.875 | 9.201 | −13.592 | 1.00 | 29.47 | O |
| ATOM | 83 | N | SER | A | 106 | 0.766 | 9.143 | −12.780 | 1.00 | 28.69 | N |
| ATOM | 84 | CA | SER | A | 106 | 1.095 | 8.456 | −11.520 | 1.00 | 29.03 | C |
| ATOM | 85 | CB | SER | A | 106 | −0.168 | 8.258 | −10.681 | 1.00 | 29.01 | C |
| ATOM | 86 | OG | SER | A | 106 | −0.803 | 9.502 | −10.472 | 1.00 | 28.27 | O |
| ATOM | 87 | C | SER | A | 106 | 1.738 | 7.095 | −11.711 | 1.00 | 30.13 | C |
| ATOM | 88 | O | SER | A | 106 | 2.626 | 6.707 | −10.946 | 1.00 | 31.08 | O |
| ATOM | 89 | N | TYR | A | 107 | 1.276 | 6.373 | −12.729 | 1.00 | 30.10 | N |
| ATOM | 90 | CA | TYR | A | 107 | 1.691 | 4.992 | −12.975 | 1.00 | 29.68 | C |
| ATOM | 91 | CB | TYR | A | 107 | 0.501 | 4.209 | −13.536 | 1.00 | 30.54 | C |
| ATOM | 92 | CG | TYR | A | 107 | −0.675 | 4.240 | −12.583 | 1.00 | 30.98 | C |
| ATOM | 93 | CD1 | TYR | A | 107 | −0.719 | 3.401 | −11.467 | 1.00 | 31.65 | C |
| ATOM | 94 | CE1 | TYR | A | 107 | −1.786 | 3.443 | −10.581 | 1.00 | 31.89 | C |
| ATOM | 95 | CZ | TYR | A | 107 | −2.820 | 4.338 | −10.798 | 1.00 | 32.28 | C |
| ATOM | 96 | OH | TYR | A | 107 | −3.882 | 4.386 | −9.926 | 1.00 | 34.34 | O |
| ATOM | 97 | CE2 | TYR | A | 107 | −2.795 | 5.179 | −11.899 | 1.00 | 32.47 | C |
| ATOM | 98 | CD2 | TYR | A | 107 | −1.723 | 5.133 | −12.777 | 1.00 | 31.49 | C |
| ATOM | 99 | C | TYR | A | 107 | 2.926 | 4.852 | −13.875 | 1.00 | 28.22 | C |
| ATOM | 100 | O | TYR | A | 107 | 3.381 | 3.737 | −14.121 | 1.00 | 29.04 | O |
| ATOM | 101 | N | GLY | A | 108 | 3.477 | 5.972 | −14.350 | 1.00 | 27.41 | N |
| ATOM | 102 | CA | GLY | A | 108 | 4.661 | 5.945 | −15.210 | 1.00 | 27.51 | C |
| ATOM | 103 | C | GLY | A | 108 | 4.389 | 5.304 | −16.559 | 1.00 | 27.61 | C |
| ATOM | 104 | O | GLY | A | 108 | 5.133 | 4.413 | −16.977 | 1.00 | 28.01 | O |
| ATOM | 105 | N | PHE | A | 109 | 3.324 | 5.761 | −17.223 | 1.00 | 26.88 | N |
| ATOM | 106 | CA | PHE | A | 109 | 2.878 | 5.229 | −18.511 | 1.00 | 26.32 | C |
| ATOM | 107 | CB | PHE | A | 109 | 1.424 | 5.649 | −18.781 | 1.00 | 26.35 | C |
| ATOM | 108 | CG | PHE | A | 109 | 0.892 | 5.226 | −20.135 | 1.00 | 26.06 | C |
| ATOM | 109 | CD1 | PHE | A | 109 | 0.881 | 3.887 | −20.498 | 1.00 | 26.38 | C |
| ATOM | 110 | CE1 | PHE | A | 109 | 0.376 | 3.488 | −21.737 | 1.00 | 26.45 | C |
| ATOM | 111 | CZ | PHE | A | 109 | −0.133 | 4.443 | −22.619 | 1.00 | 26.40 | C |
| ATOM | 112 | CE2 | PHE | A | 109 | −0.132 | 5.787 | −22.261 | 1.00 | 25.52 | C |
| ATOM | 113 | CD2 | PHE | A | 109 | 0.372 | 6.168 | −21.031 | 1.00 | 25.12 | C |
| ATOM | 114 | C | PHE | A | 109 | 3.767 | 5.704 | −19.656 | 1.00 | 26.01 | C |
| ATOM | 115 | O | PHE | A | 109 | 3.920 | 6.910 | −19.863 | 1.00 | 25.89 | O |
| ATOM | 116 | N | ARG | A | 110 | 4.357 | 4.762 | −20.398 | 1.00 | 25.91 | N |
| ATOM | 117 | CA | ARG | A | 110 | 5.033 | 5.077 | −21.661 | 1.00 | 25.87 | C |
| ATOM | 118 | CB | ARG | A | 110 | 6.555 | 5.313 | −21.464 | 1.00 | 27.84 | C |
| ATOM | 119 | CG | ARG | A | 110 | 7.234 | 4.222 | −20.722 | 1.00 | 29.95 | C |
| ATOM | 120 | CD | ARG | A | 110 | 8.602 | 4.609 | −20.115 | 1.00 | 32.82 | C |
| ATOM | 121 | NE | ARG | A | 110 | 9.252 | 3.388 | −19.630 | 1.00 | 34.27 | N |
| ATOM | 122 | CZ | ARG | A | 110 | 8.933 | 2.725 | −18.511 | 1.00 | 34.79 | C |
| ATOM | 123 | NH1 | ARG | A | 110 | 7.929 | 3.155 | −17.694 | 1.00 | 35.06 | N |
| ATOM | 124 | NH2 | ARG | A | 110 | 9.578 | 1.598 | −18.222 | 1.00 | 35.60 | N |
| ATOM | 125 | C | ARG | A | 110 | 4.788 | 4.004 | −22.694 | 1.00 | 24.24 | C |
| ATOM | 126 | O | ARG | A | 110 | 4.329 | 2.929 | −22.373 | 1.00 | 23.22 | O |
| ATOM | 127 | N | LEU | A | 111 | 5.091 | 4.319 | −23.948 | 1.00 | 23.32 | N |
| ATOM | 128 | CA | LEU | A | 111 | 4.946 | 3.407 | −25.073 | 1.00 | 22.67 | C |
| ATOM | 129 | CB | LEU | A | 111 | 4.358 | 4.121 | −26.286 | 1.00 | 22.29 | C |
| ATOM | 130 | CG | LEU | A | 111 | 2.963 | 4.708 | −26.148 | 1.00 | 22.51 | C |
| ATOM | 131 | CD1 | LEU | A | 111 | 2.625 | 5.468 | −27.412 | 1.00 | 22.92 | C |
| ATOM | 132 | CD2 | LEU | A | 111 | 1.950 | 3.606 | −25.895 | 1.00 | 23.41 | C |
| ATOM | 133 | C | LEU | A | 111 | 6.282 | 2.792 | −25.465 | 1.00 | 22.10 | C |
| ATOM | 134 | O | LEU | A | 111 | 7.334 | 3.411 | −25.312 | 1.00 | 21.74 | O |
| ATOM | 135 | N | GLY | A | 112 | 6.204 | 1.560 | −25.970 | 1.00 | 20.85 | N |
| ATOM | 136 | CA | GLY | A | 112 | 7.341 | 0.849 | −26.528 | 1.00 | 20.23 | C |
| ATOM | 137 | C | GLY | A | 112 | 6.966 | 0.265 | −27.877 | 1.00 | 19.86 | C |
| ATOM | 138 | O | GLY | A | 112 | 5.785 | 0.086 | −28.192 | 1.00 | 19.47 | O |
| ATOM | 139 | N | PHE | A | 113 | 7.990 | −0.026 | −28.671 | 1.00 | 18.89 | N |
| ATOM | 140 | CA | PHE | A | 113 | 7.817 | −0.561 | −30.012 | 1.00 | 19.30 | C |
| ATOM | 141 | CB | PHE | A | 113 | 7.971 | 0.562 | −31.044 | 1.00 | 19.64 | C |
| ATOM | 142 | CG | PHE | A | 113 | 7.124 | 1.761 | −30.744 | 1.00 | 21.02 | C |
| ATOM | 143 | CD1 | PHE | A | 113 | 5.785 | 1.804 | −31.146 | 1.00 | 21.06 | C |
| ATOM | 144 | CE1 | PHE | A | 113 | 4.995 | 2.900 | −30.850 | 1.00 | 21.39 | C |
| ATOM | 145 | CZ | PHE | A | 113 | 5.525 | 3.962 | −30.125 | 1.00 | 21.27 | C |
| ATOM | 146 | CE2 | PHE | A | 113 | 6.848 | 3.925 | −29.720 | 1.00 | 21.60 | C |
| ATOM | 147 | CD2 | PHE | A | 113 | 7.638 | 2.827 | −30.021 | 1.00 | 21.42 | C |
| ATOM | 148 | C | PHE | A | 113 | 8.851 | −1.650 | −30.233 | 1.00 | 19.21 | C |
| ATOM | 149 | O | PHE | A | 113 | 9.919 | −1.610 | −29.629 | 1.00 | 19.02 | O |
| ATOM | 150 | N | LEU | A | 114 | 8.535 | −2.620 | −31.086 | 1.00 | 18.88 | N |
| ATOM | 151 | CA | LEU | A | 114 | 9.505 | −3.655 | −31.435 | 1.00 | 19.56 | C |
| ATOM | 152 | CB | LEU | A | 114 | 8.882 | −4.779 | −32.259 | 1.00 | 19.11 | C |
| ATOM | 153 | CG | LEU | A | 114 | 7.773 | −5.636 | −31.668 | 1.00 | 18.84 | C |
| ATOM | 154 | CD1 | LEU | A | 114 | 7.309 | −6.652 | −32.701 | 1.00 | 18.43 | C |
| ATOM | 155 | CD2 | LEU | A | 114 | 8.240 | −6.337 | −30.401 | 1.00 | 18.72 | C |
| ATOM | 156 | C | LEU | A | 114 | 10.640 | −3.011 | −32.240 | 1.00 | 20.45 | C |
| ATOM | 157 | O | LEU | A | 114 | 10.416 | −2.009 | −32.947 | 1.00 | 20.91 | O |
| ATOM | 158 | N | HIS | A | 115 | 11.855 | −3.559 | −32.100 | 1.00 | 20.64 | N |
| ATOM | 159 | CA | HIS | A | 115 | 13.020 | −3.079 | −32.827 | 1.00 | 20.72 | C |
| ATOM | 160 | CB | HIS | A | 115 | 14.240 | −3.066 | −31.787 | 1.00 | 21.07 | C |
| ATOM | 161 | CG | HIS | A | 115 | 14.005 | −2.297 | −30.638 | 1.00 | 21.51 | C |
| ATOM | 162 | ND1 | HIS | A | 115 | 13.910 | −2.906 | −29.405 | 1.00 | 21.49 | N |
| ATOM | 163 | CE1 | HIS | A | 115 | 13.699 | −1.986 | −28.479 | 1.00 | 21.50 | C |
| ATOM | 164 | NE2 | HIS | A | 115 | 13.627 | −0.805 | −29.070 | 1.00 | 21.33 | N |
| ATOM | 165 | CD2 | HIS | A | 115 | 13.798 | −0.973 | −30.422 | 1.00 | 21.73 | C |
| ATOM | 166 | C | HIS | A | 115 | 13.217 | −3.940 | −34.066 | 1.00 | 21.11 | C |
| ATOM | 167 | O | HIS | A | 115 | 14.055 | −4.837 | −34.101 | 1.00 | 21.22 | O |
| ATOM | 168 | N | SER | A | 116 | 12.421 | −3.645 | −35.087 | 1.00 | 21.64 | N |
| ATOM | 169 | CA | SER | A | 116 | 12.196 | −4.553 | −36.205 | 1.00 | 21.17 | C |
| ATOM | 170 | CB | SER | A | 116 | 10.737 | −4.439 | −36.654 | 1.00 | 22.26 | C |
| ATOM | 171 | OG | SER | A | 116 | 9.864 | −4.694 | −35.567 | 1.00 | 22.55 | O |
| ATOM | 172 | C | SER | A | 116 | 13.122 | −4.339 | −37.396 | 1.00 | 21.47 | C |
| ATOM | 173 | O | SER | A | 116 | 13.213 | −5.235 | −38.257 | 1.00 | 21.22 | O |
| ATOM | 174 | N | GLY | A | 117 | 13.780 | −3.175 | −37.472 | 1.00 | 20.22 | N |
| ATOM | 175 | CA | GLY | A | 117 | 14.758 | −2.923 | −38.538 | 1.00 | 19.73 | C |
| ATOM | 176 | C | GLY | A | 117 | 14.072 | −2.500 | −39.824 | 1.00 | 19.41 | C |
| ATOM | 177 | O | GLY | A | 117 | 12.851 | −2.337 | −39.849 | 1.00 | 19.54 | O |
| ATOM | 178 | N | THR | A | 118 | 14.847 | −2.329 | −40.887 | 1.00 | 19.45 | N |
| ATOM | 179 | CA | THR | A | 118 | 14.317 | −1.796 | −42.154 | 1.00 | 20.80 | C |
| ATOM | 180 | CB | THR | A | 118 | 14.893 | −0.396 | −42.473 | 1.00 | 20.39 | C |
| ATOM | 181 | OG1 | THR | A | 118 | 16.321 | −0.448 | −42.524 | 1.00 | 19.77 | O |
| ATOM | 182 | CG2 | THR | A | 118 | 14.487 | 0.577 | −41.438 | 1.00 | 19.99 | C |
| ATOM | 183 | C | THR | A | 118 | 14.521 | −2.728 | −43.353 | 1.00 | 21.83 | C |
| ATOM | 184 | O | THR | A | 118 | 14.537 | −2.271 | −44.495 | 1.00 | 21.26 | O |
| ATOM | 185 | N | ALA | A | 119 | 14.632 | −4.034 | −43.091 | 1.00 | 24.00 | N |
| ATOM | 186 | CA | ALA | A | 119 | 14.684 | −5.030 | −44.150 | 1.00 | 25.79 | C |
| ATOM | 187 | CB | ALA | A | 119 | 14.882 | −6.425 | −43.564 | 1.00 | 25.77 | C |
| ATOM | 188 | C | ALA | A | 119 | 13.409 | −4.982 | −44.999 | 1.00 | 28.02 | C |
| ATOM | 189 | O | ALA | A | 119 | 12.312 | −4.722 | −44.481 | 1.00 | 28.20 | O |
| ATOM | 190 | N | LYS | A | 120 | 13.580 | −5.238 | −46.299 | 1.00 | 30.32 | N |
| ATOM | 191 | CA | LYS | A | 120 | 12.496 | −5.121 | −47.297 | 1.00 | 32.05 | C |
| ATOM | 192 | CB | LYS | A | 120 | 13.007 | −5.570 | −48.683 | 1.00 | 34.83 | C |
| ATOM | 193 | CG | LYS | A | 120 | 12.550 | −4.738 | −49.887 | 1.00 | 37.72 | C |
| ATOM | 194 | CD | LYS | A | 120 | 13.534 | −4.859 | −51.054 | 1.00 | 39.67 | C |
| ATOM | 195 | CE | LYS | A | 120 | 14.720 | −3.909 | −50.895 | 1.00 | 40.64 | C |
| ATOM | 196 | NZ | LYS | A | 120 | 15.701 | −4.052 | −52.002 | 1.00 | 40.71 | N |
| ATOM | 197 | C | LYS | A | 120 | 11.234 | −5.918 | −46.922 | 1.00 | 32.06 | C |
| ATOM | 198 | O | LYS | A | 120 | 10.128 | −5.489 | −47.215 | 1.00 | 32.50 | O |
| ATOM | 199 | N | SER | A | 121 | 11.414 | −7.065 | −46.267 | 1.00 | 33.07 | N |
| ATOM | 200 | CA | SER | A | 121 | 10.312 | −7.939 | −45.855 | 1.00 | 34.60 | C |
| ATOM | 201 | CB | SER | A | 121 | 10.868 | −9.342 | −45.595 | 1.00 | 34.59 | C |
| ATOM | 202 | OG | SER | A | 121 | 11.698 | −9.351 | −44.443 | 1.00 | 34.98 | O |
| ATOM | 203 | C | SER | A | 121 | 9.516 | −7.490 | −44.605 | 1.00 | 35.65 | C |
| ATOM | 204 | O | SER | A | 121 | 8.591 | −8.203 | −44.193 | 1.00 | 36.14 | O |
| ATOM | 205 | N | VAL | A | 122 | 9.870 | −6.349 | −44.000 | 1.00 | 35.34 | N |
| ATOM | 206 | CA | VAL | A | 122 | 9.261 | −5.939 | −42.737 | 1.00 | 35.33 | C |
| ATOM | 207 | CB | VAL | A | 122 | 10.209 | −4.998 | −41.933 | 1.00 | 37.44 | C |
| ATOM | 208 | CG1 | VAL | A | 122 | 10.170 | −3.557 | −42.439 | 1.00 | 37.52 | C |
| ATOM | 209 | CG2 | VAL | A | 122 | 9.898 | −5.033 | −40.449 | 1.00 | 38.16 | C |
| ATOM | 210 | C | VAL | A | 122 | 7.868 | −5.335 | −42.989 | 1.00 | 33.86 | C |
| ATOM | 211 | O | VAL | A | 122 | 7.700 | −4.486 | −43.860 | 1.00 | 35.32 | O |
| ATOM | 212 | N | THR | A | 123 | 6.870 | −5.815 | −42.252 | 1.00 | 31.47 | N |
| ATOM | 213 | CA | THR | A | 123 | 5.484 | −5.342 | −42.385 | 1.00 | 30.23 | C |
| ATOM | 214 | CB | THR | A | 123 | 4.475 | −6.456 | −42.074 | 1.00 | 30.48 | C |
| ATOM | 215 | OG1 | THR | A | 123 | 4.708 | −6.960 | −40.763 | 1.00 | 31.07 | O |
| ATOM | 216 | CG2 | THR | A | 123 | 4.617 | −7.587 | −43.083 | 1.00 | 31.73 | C |
| ATOM | 217 | C | THR | A | 123 | 5.182 | −4.147 | −41.480 | 1.00 | 28.98 | C |
| ATOM | 218 | O | THR | A | 123 | 4.266 | −3.366 | −41.780 | 1.00 | 28.58 | O |
| ATOM | 219 | N | CYS | A | 124 | 5.937 | −4.021 | −40.386 | 1.00 | 26.87 | N |
| ATOM | 220 | CA | CYS | A | 124 | 5.766 | −2.933 | −39.428 | 1.00 | 26.51 | C |
| ATOM | 221 | CB | CYS | A | 124 | 4.772 | −3.398 | −38.351 | 1.00 | 27.68 | C |
| ATOM | 222 | SG | CYS | A | 124 | 4.383 | −2.174 | −37.102 | 1.00 | 30.56 | S |
| ATOM | 223 | C | CYS | A | 124 | 7.118 | −2.565 | −38.791 | 1.00 | 24.79 | C |
| ATOM | 224 | O | CYS | A | 124 | 7.844 | −3.451 | −38.334 | 1.00 | 23.89 | O |
| ATOM | 225 | N | THR | A | 125 | 7.453 | −1.276 | −38.774 | 1.00 | 22.85 | N |
| ATOM | 226 | CA | THR | A | 125 | 8.708 | −0.807 | −38.166 | 1.00 | 22.23 | C |
| ATOM | 227 | CB | THR | A | 125 | 9.924 | −0.927 | −39.124 | 1.00 | 21.98 | C |
| ATOM | 228 | OG1 | THR | A | 125 | 11.130 | −0.523 | −38.452 | 1.00 | 20.92 | O |
| ATOM | 229 | CG2 | THR | A | 125 | 9.750 | −0.074 | −40.381 | 1.00 | 22.30 | C |
| ATOM | 230 | C | THR | A | 125 | 8.593 | 0.626 | −37.645 | 1.00 | 22.09 | C |
| ATOM | 231 | O | THR | A | 125 | 7.954 | 1.478 | −38.276 | 1.00 | 23.52 | O |
| ATOM | 232 | N | TYR | A | 126 | 9.243 | 0.873 | −36.506 | 1.00 | 21.01 | N |
| ATOM | 233 | CA | TYR | A | 126 | 9.196 | 2.152 | −35.811 | 1.00 | 19.96 | C |
| ATOM | 234 | CB | TYR | A | 126 | 8.607 | 1.914 | −34.355 | 1.00 | 20.14 | C |
| ATOM | 235 | CG | TYR | A | 126 | 8.665 | 3.137 | −33.478 | 1.00 | 20.21 | C |
| ATOM | 236 | CD1 | TYR | A | 126 | 7.884 | 4.127 | −33.556 | 1.00 | 20.52 | C |
| ATOM | 237 | CE1 | TYR | A | 126 | 7.934 | 5.254 | −32.741 | 1.00 | 20.39 | C |
| ATOM | 238 | CZ | TYR | A | 126 | 8.977 | 5.393 | −31.846 | 1.00 | 20.56 | C |
| ATOM | 239 | OH | TYR | A | 126 | 9.037 | 6.491 | −31.032 | 1.00 | 20.22 | O |
| ATOM | 240 | CE2 | TYR | A | 126 | 9.960 | 4.427 | −31.753 | 1.00 | 20.07 | C |
| ATOM | 241 | CD2 | TYR | A | 126 | 9.898 | 3.308 | −32.566 | 1.00 | 19.94 | C |
| ATOM | 242 | C | TYR | A | 126 | 10.541 | 2.849 | −35.873 | 1.00 | 19.25 | C |
| ATOM | 243 | O | TYR | A | 126 | 11.566 | 2.232 | −35.649 | 1.00 | 19.18 | O |
| ATOM | 244 | N | SER | A | 127 | 10.515 | 4.145 | −36.169 | 1.00 | 18.85 | N |
| ATOM | 245 | CA | SER | A | 127 | 11.704 | 5.006 | −36.200 | 1.00 | 18.11 | C |
| ATOM | 246 | CB | SER | A | 127 | 11.610 | 5.976 | −37.376 | 1.00 | 18.35 | C |
| ATOM | 247 | OG | SER | A | 127 | 12.559 | 7.001 | −37.261 | 1.00 | 18.80 | O |
| ATOM | 248 | C | SER | A | 127 | 11.766 | 5.802 | −34.909 | 1.00 | 17.59 | C |
| ATOM | 249 | O | SER | A | 127 | 10.929 | 6.671 | −34.704 | 1.00 | 17.27 | O |
| ATOM | 250 | N | PRO | A | 128 | 12.746 | 5.513 | −34.030 | 1.00 | 17.21 | N |
| ATOM | 251 | CA | PRO | A | 128 | 12.918 | 6.358 | −32.835 | 1.00 | 17.07 | C |
| ATOM | 252 | CB | PRO | A | 128 | 14.070 | 5.682 | −32.088 | 1.00 | 16.98 | C |
| ATOM | 253 | CG | PRO | A | 128 | 14.016 | 4.254 | −32.512 | 1.00 | 16.95 | C |
| ATOM | 254 | CD | PRO | A | 128 | 13.525 | 4.257 | −33.927 | 1.00 | 16.99 | C |
| ATOM | 255 | C | PRO | A | 128 | 13.267 | 7.816 | −33.154 | 1.00 | 17.04 | C |
| ATOM | 256 | O | PRO | A | 128 | 12.792 | 8.707 | −32.470 | 1.00 | 17.45 | O |
| ATOM | 257 | N | ALA | A | 129 | 14.072 | 8.051 | −34.185 | 1.00 | 17.39 | N |
| ATOM | 258 | CA | ALA | A | 129 | 14.497 | 9.412 | −34.532 | 1.00 | 17.72 | C |
| ATOM | 259 | CB | ALA | A | 129 | 15.549 | 9.381 | −35.627 | 1.00 | 17.25 | C |
| ATOM | 260 | C | ALA | A | 129 | 13.328 | 10.302 | −34.945 | 1.00 | 18.26 | C |
| ATOM | 261 | O | ALA | A | 129 | 13.288 | 11.477 | −34.596 | 1.00 | 18.85 | O |
| ATOM | 262 | N | LEU | A | 130 | 12.376 | 9.721 | −35.671 | 1.00 | 18.82 | N |
| ATOM | 263 | CA | LEU | A | 130 | 11.194 | 10.439 | −36.154 | 1.00 | 19.75 | C |
| ATOM | 264 | CB | LEU | A | 130 | 10.865 | 10.002 | −37.587 | 1.00 | 20.08 | C |
| ATOM | 265 | CG | LEU | A | 130 | 11.881 | 10.360 | −38.678 | 1.00 | 20.56 | C |
| ATOM | 266 | CD1 | LEU | A | 130 | 11.668 | 9.509 | −39.937 | 1.00 | 20.71 | C |
| ATOM | 267 | CD2 | LEU | A | 130 | 11.873 | 11.857 | −39.018 | 1.00 | 20.57 | C |
| ATOM | 268 | C | LEU | A | 130 | 9.950 | 10.254 | −35.268 | 1.00 | 20.08 | C |
| ATOM | 269 | O | LEU | A | 130 | 8.949 | 10.934 | −35.480 | 1.00 | 20.12 | O |
| ATOM | 270 | N | ASN | A | 131 | 10.007 | 9.352 | −34.286 | 1.00 | 20.34 | N |
| ATOM | 271 | CA | ASN | A | 131 | 8.829 | 8.916 | −33.497 | 1.00 | 20.38 | C |
| ATOM | 272 | CB | ASN | A | 131 | 8.446 | 9.983 | −32.458 | 1.00 | 19.91 | C |
| ATOM | 273 | CG | ASN | A | 131 | 7.359 | 9.516 | −31.498 | 1.00 | 20.09 | C |
| ATOM | 274 | OD1 | ASN | A | 131 | 7.246 | 8.317 | −31.192 | 1.00 | 20.18 | O |
| ATOM | 275 | ND2 | ASN | A | 131 | 6.540 | 10.465 | −31.031 | 1.00 | 19.02 | N |
| ATOM | 276 | C | ASN | A | 131 | 7.669 | 8.589 | −34.445 | 1.00 | 20.49 | C |
| ATOM | 277 | O | ASN | A | 131 | 6.553 | 9.100 | −34.317 | 1.00 | 20.55 | O |
| ATOM | 278 | N | LYS | A | 132 | 7.971 | 7.736 | −35.415 | 1.00 | 20.63 | N |
| ATOM | 279 | CA | LYS | A | 132 | 7.074 | 7.458 | −36.530 | 1.00 | 20.29 | C |
| ATOM | 280 | CB | LYS | A | 132 | 7.548 | 8.201 | −37.767 | 1.00 | 20.77 | C |
| ATOM | 281 | CG | LYS | A | 132 | 6.563 | 8.182 | −38.919 | 1.00 | 21.51 | C |
| ATOM | 282 | CD | LYS | A | 132 | 6.974 | 9.196 | −39.968 | 1.00 | 21.84 | C |
| ATOM | 283 | CE | LYS | A | 132 | 6.076 | 9.128 | −41.187 | 1.00 | 22.52 | C |
| ATOM | 284 | NZ | LYS | A | 132 | 6.346 | 10.240 | −42.137 | 1.00 | 23.49 | N |
| ATOM | 285 | C | LYS | A | 132 | 6.997 | 5.964 | −36.810 | 1.00 | 19.90 | C |
| ATOM | 286 | O | LYS | A | 132 | 8.015 | 5.306 | −36.995 | 1.00 | 18.84 | O |
| ATOM | 287 | N | MET | A | 133 | 5.775 | 5.447 | −36.843 | 1.00 | 20.31 | N |
| ATOM | 288 | CA | MET | A | 133 | 5.516 | 4.063 | −37.187 | 1.00 | 21.26 | C |
| ATOM | 289 | CB | MET | A | 133 | 4.309 | 3.566 | −36.413 | 1.00 | 22.67 | C |
| ATOM | 290 | CG | MET | A | 133 | 3.914 | 2.123 | −36.697 | 1.00 | 24.99 | C |
| ATOM | 291 | SD | MET | A | 133 | 5.076 | 0.934 | −36.006 | 1.00 | 28.44 | S |
| ATOM | 292 | CE | MET | A | 133 | 4.575 | 0.999 | −34.270 | 1.00 | 26.66 | C |
| ATOM | 293 | C | MET | A | 133 | 5.265 | 3.989 | −38.704 | 1.00 | 20.85 | C |
| ATOM | 294 | O | MET | A | 133 | 4.530 | 4.803 | −39.263 | 1.00 | 19.44 | O |
| ATM | 295 | N | PHE | A | 134 | 5.895 | 3.009 | −39.355 | 1.00 | 19.93 | N |
| ATOM | 296 | CA | PHE | A | 134 | 5.647 | 2.695 | −40.753 | 1.00 | 19.71 | C |
| ATOM | 297 | CB | PHE | A | 134 | 6.949 | 2.722 | −41.548 | 1.00 | 19.74 | C |
| ATOM | 298 | CG | PHE | A | 134 | 7.620 | 4.067 | −41.580 | 1.00 | 19.77 | C |
| ATOM | 299 | CD1 | PHE | A | 134 | 8.374 | 4.511 | −40.493 | 1.00 | 19.87 | C |
| ATOM | 300 | CE1 | PHE | A | 134 | 8.988 | 5.752 | −40.515 | 1.00 | 20.54 | C |
| ATOM | 301 | CZ | PHE | A | 134 | 8.663 | 6.577 | −41.638 | 1.00 | 20.71 | C |
| ATOM | 302 | CE2 | PHE | A | 134 | 8.117 | 6.144 | −42.719 | 1.00 | 20.82 | C |
| ATOM | 303 | CD2 | PHE | A | 134 | 7.505 | 4.892 | −42.693 | 1.00 | 20.27 | C |
| ATOM | 304 | C | PHE | A | 134 | 5.032 | 1.298 | −40.792 | 1.00 | 20.04 | C |
| ATOM | 305 | O | PHE | A | 134 | 5.648 | 0.348 | −40.298 | 1.00 | 19.56 | O |
| ATOM | 306 | N | CYS | A | 135 | 3.819 | 1.176 | −41.347 | 1.00 | 20.98 | N |
| ATOM | 307 | CA | CYS | A | 135 | 3.147 | −0.118 | −41.460 | 1.00 | 21.78 | C |
| ATOM | 308 | CB | CYS | A | 135 | 2.108 | −0.333 | −40.356 | 1.00 | 23.13 | C |
| ATOM | 309 | SG | CYS | A | 135 | 0.973 | 1.030 | −40.179 | 1.00 | 25.34 | S |
| ATOM | 310 | C | CYS | A | 135 | 2.456 | −0.314 | −42.798 | 1.00 | 22.04 | C |
| ATOM | 311 | O | CYS | A | 135 | 2.129 | 0.651 | −43.500 | 1.00 | 21.98 | O |
| ATOM | 312 | N | GLN | A | 136 | 2.230 | −1.584 | −43.129 | 1.00 | 22.94 | N |
| ATOM | 313 | CA | GLN | A | 136 | 1.416 | −1.957 | −44.272 | 1.00 | 23.88 | C |
| ATOM | 314 | CB | GLN | A | 136 | 1.835 | −3.324 | −44.802 | 1.00 | 25.24 | C |
| ATOM | 315 | CG | GLN | A | 136 | 3.124 | −3.293 | −45.626 | 1.00 | 26.88 | C |
| ATOM | 316 | CD | GLN | A | 136 | 3.494 | −4.661 | −46.191 | 1.00 | 28.43 | C |
| ATOM | 317 | OE1 | GLN | A | 136 | 3.199 | −5.684 | −45.600 | 1.00 | 29.60 | O |
| ATOM | 318 | NE2 | GLN | A | 136 | 4.140 | −4.673 | −47.359 | 1.00 | 30.13 | N |
| ATOM | 319 | C | GLN | A | 136 | −0.073 | −1.918 | −43.907 | 1.00 | 23.33 | C |
| ATOM | 320 | O | GLN | A | 136 | −0.436 | −2.141 | −42.740 | 1.00 | 22.36 | O |
| ATOM | 321 | N | LEU | A | 137 | −0.908 | −1.629 | −44.898 | 1.00 | 23.60 | N |
| ATOM | 322 | CA | LEU | A | 137 | −2.349 | −1.513 | −44.706 | 1.00 | 24.34 | C |
| ATOM | 323 | CB | LEU | A | 137 | −3.060 | −1.071 | −46.002 | 1.00 | 25.04 | C |
| ATOM | 324 | CG | LEU | A | 137 | −4.580 | −0.818 | −45.825 | 1.00 | 26.23 | C |
| ATOM | 325 | CD1 | LEU | A | 137 | −5.081 | 0.574 | −46.232 | 1.00 | 25.98 | C |
| ATOM | 326 | CD2 | LEU | A | 137 | −5.384 | −1.952 | −46.471 | 1.00 | 27.00 | C |
| ATOM | 327 | C | LEU | A | 137 | −2.964 | −2.805 | −44.172 | 1.00 | 24.77 | C |
| ATOM | 328 | O | LEU | A | 137 | −2.707 | −3.869 | −44.704 | 1.00 | 23.81 | O |
| ATOM | 329 | N | ALA | A | 138 | −3.749 | −2.681 | −43.092 | 1.00 | 25.29 | N |
| ATOM | 330 | CA | ALA | A | 138 | −4.452 | −3.801 | −42.462 | 1.00 | 26.21 | C |
| ATOM | 331 | CB | ALA | A | 138 | −5.472 | −4.420 | −43.437 | 1.00 | 26.60 | C |
| ATOM | 332 | C | ALA | A | 138 | −3.552 | −4.909 | −41.885 | 1.00 | 26.15 | C |
| ATOM | 333 | O | ALA | A | 138 | −4.013 | −6.031 | −41.681 | 1.00 | 27.20 | O |
| ATOM | 334 | N | LYS | A | 139 | −2.287 | −4.604 | −41.610 | 1.00 | 25.65 | N |
| ATOM | 335 | CA | LYS | A | 139 | −1.364 | −5.579 | −41.023 | 1.00 | 26.48 | C |
| ATOM | 336 | CB | LYS | A | 139 | 0.002 | −5.538 | −41.708 | 1.00 | 27.71 | C |
| ATOM | 337 | CG | LYS | A | 139 | −0.040 | −5.891 | −43.194 | 1.00 | 28.84 | C |
| ATOM | 338 | CD | LYS | A | 139 | −0.295 | −7.370 | −43.500 | 1.00 | 29.36 | C |
| ATOM | 339 | CE | LYS | A | 139 | −1.079 | −7.546 | −44.805 | 1.00 | 30.26 | C |
| ATOM | 340 | NZ | LYS | A | 139 | −1.631 | −8.922 | −44.881 | 1.00 | 31.49 | N |
| ATOM | 341 | C | LYS | A | 139 | −1.187 | −5.282 | −39.543 | 1.00 | 26.02 | C |
| ATOM | 342 | O | LYS | A | 139 | −1.377 | −4.144 | −39.093 | 1.00 | 24.90 | O |
| ATOM | 343 | N | THR | A | 140 | −0.820 | −6.323 | −38.797 | 1.00 | 25.37 | N |
| ATOM | 344 | CA | THR | A | 140 | −0.537 | −6.226 | −37.372 | 1.00 | 25.66 | C |
| ATOM | 345 | CB | THR | A | 140 | −0.009 | −7.562 | −36.827 | 1.00 | 25.21 | C |
| ATOM | 346 | OG1 | THR | A | 140 | −0.912 | −8.607 | −37.194 | 1.00 | 24.79 | O |
| ATOM | 347 | OG2 | THR | A | 140 | 0.126 | −7.527 | −35.309 | 1.00 | 25.38 | C |
| ATOM | 348 | C | THR | A | 140 | 0.508 | −5.145 | −37.090 | 1.00 | 26.08 | C |
| ATOM | 349 | O | THR | A | 140 | 1.571 | −5.091 | −37.727 | 1.00 | 24.90 | O |
| ATOM | 350 | N | CYS | A | 141 | 0.185 | −4.285 | −36.129 | 1.00 | 27.39 | N |
| ATOM | 351 | CA | CYS | A | 141 | 1.061 | −3.205 | −35.710 | 1.00 | 27.90 | C |
| ATOM | 352 | CB | CYS | A | 141 | 0.519 | −1.880 | −36.244 | 1.00 | 29.32 | C |
| ATOM | 353 | SG | CYS | A | 141 | 1.648 | −0.494 | −36.114 | 1.00 | 34.50 | S |
| ATOM | 354 | C | CYS | A | 141 | 1.110 | −3.247 | −34.183 | 1.00 | 27.05 | C |
| ATOM | 355 | O | CYS | A | 141 | 0.216 | −2.721 | −33.518 | 1.00 | 27.59 | O |
| ATOM | 356 | N | PRO | A | 142 | 2.130 | −3.929 | −33.612 | 1.00 | 25.82 | N |
| ATOM | 357 | CA | PRO | A | 142 | 2.187 | −4.022 | −32.153 | 1.00 | 25.28 | C |
| ATOM | 358 | CB | PRO | A | 142 | 3.245 | −5.110 | −31.889 | 1.00 | 24.99 | C |
| ATOM | 359 | CG | PRO | A | 142 | 3.670 | −5.620 | −33.212 | 1.00 | 24.69 | C |
| ATOM | 360 | CD | PRO | A | 142 | 3.261 | −4.633 | −34.244 | 1.00 | 24.99 | C |
| ATOM | 361 | C | PRO | A | 142 | 2.603 | −2.701 | −31.509 | 1.00 | 24.22 | C |
| ATOM | 362 | O | PRO | A | 142 | 3.545 | −2.057 | −31.984 | 1.00 | 24.69 | O |
| ATOM | 363 | N | VAL | A | 143 | 1.884 | −2.306 | −30.462 | 1.00 | 23.73 | N |
| ATOM | 364 | CA | VAL | A | 143 | 2.258 | −1.171 | −29.628 | 1.00 | 23.65 | C |
| ATOM | 365 | CB | VAL | A | 143 | 1.286 | 0.017 | −29.778 | 1.00 | 23.37 | C |
| ATOM | 366 | CG1 | VAL | A | 143 | 1.593 | 1.122 | −28.778 | 1.00 | 23.00 | C |
| ATOM | 367 | CG2 | VAL | A | 143 | 1.321 | 0.550 | −31.207 | 1.00 | 23.54 | C |
| ATOM | 368 | C | VAL | A | 143 | 2.292 | −1.670 | −28.186 | 1.00 | 23.27 | C |
| ATOM | 369 | O | VAL | A | 143 | 1.353 | −2.329 | −27.728 | 1.00 | 22.47 | O |
| ATOM | 370 | N | GLN | A | 144 | 3.392 | −1.364 | −27.490 | 1.00 | 23.03 | N |
| ATOM | 371 | CA | GLN | A | 144 | 3.622 | −1.838 | −26.129 | 1.00 | 23.49 | C |
| ATOM | 372 | CB | GLN | A | 144 | 5.087 | −2.232 | −25.944 | 1.00 | 23.18 | C |
| ATOM | 373 | CG | GLN | A | 144 | 5.595 | −3.295 | −26.916 | 1.00 | 22.44 | C |
| ATOM | 374 | CD | GLN | A | 144 | 7.087 | −3.542 | −26.807 | 1.00 | 21.71 | C |
| ATOM | 375 | OE1 | GLN | A | 144 | 7.832 | −2.722 | −26.276 | 1.00 | 20.59 | O |
| ATOM | 376 | NE2 | GLN | A | 144 | 7.529 | −4.689 | −27.307 | 1.00 | 20.95 | N |
| ATOM | 377 | C | GLN | A | 144 | 3.267 | −0.761 | −25.117 | 1.00 | 24.26 | C |
| ATOM | 378 | O | GLN | A | 144 | 3.551 | 0.413 | −25.337 | 1.00 | 25.03 | O |
| ATOM | 379 | N | LEU | A | 145 | 2.656 | −1.178 | −24.010 | 1.00 | 24.86 | N |
| ATOM | 380 | CA | LEU | A | 145 | 2.355 | −0.308 | −22.883 | 1.00 | 24.78 | C |
| ATOM | 381 | CB | LEU | A | 145 | 0.909 | −0.509 | −22.429 | 1.00 | 24.16 | C |
| ATOM | 382 | CG | LEU | A | 145 | −0.148 | −0.591 | −23.529 | 1.00 | 23.70 | C |
| ATOM | 383 | CD1 | LEU | A | 145 | −1.526 | −0.714 | −22.889 | 1.00 | 23.19 | C |
| ATOM | 384 | CD2 | LEU | A | 145 | −0.143 | 0.624 | −24.448 | 1.00 | 23.63 | C |
| ATOM | 385 | C | LEU | A | 145 | 3.325 | −0.667 | −21.755 | 1.00 | 26.38 | C |
| ATOM | 386 | O | LEU | A | 145 | 3.361 | −1.810 | −21.288 | 1.00 | 26.32 | O |
| ATOM | 387 | N | TRP | A | 146 | 4.121 | 0.310 | −21.339 | 1.00 | 28.19 | N |
| ATOM | 388 | CA | TRP | A | 146 | 5.075 | 0.146 | −20.246 | 1.00 | 28.76 | C |
| ATOM | 389 | CB | TRP | A | 146 | 6.479 | 0.559 | −20.694 | 1.00 | 28.46 | C |
| ATOM | 390 | CG | TRP | A | 146 | 7.111 | −0.360 | −21.711 | 1.00 | 28.27 | C |
| ATOM | 391 | CD1 | TRP | A | 146 | 6.806 | −0.455 | −23.036 | 1.00 | 27.81 | C |
| ATOM | 392 | NE1 | TRP | A | 146 | 7.598 | −1.402 | −23.637 | 1.00 | 28.02 | N |
| ATOM | 393 | CE2 | TRP | A | 146 | 8.450 | −1.934 | −22.706 | 1.00 | 28.64 | C |
| ATOM | 394 | CD2 | TRP | A | 146 | 8.173 | −1.298 | −21.475 | 1.00 | 28.94 | C |
| ATOM | 395 | CE3 | TRP | A | 146 | 8.931 | −1.650 | −20.344 | 1.00 | 29.13 | C |
| ATOM | 396 | CZ3 | TRP | A | 146 | 9.892 | −2.649 | −20.463 | 1.00 | 29.12 | C |
| ATOM | 397 | CH2 | TRP | A | 146 | 10.153 | −3.265 | −21.713 | 1.00 | 29.79 | C |
| ATOM | 398 | CZ2 | TRP | A | 146 | 9.439 | −2.922 | −22.840 | 1.00 | 29.16 | C |
| ATOM | 399 | C | TRP | A | 146 | 4.619 | 1.028 | −19.089 | 1.00 | 30.60 | C |
| ATOM | 400 | O | TRP | A | 146 | 4.370 | 2.222 | −19.291 | 1.00 | 31.91 | O |
| ATOM | 401 | N | VAL | A | 147 | 4.511 | 0.435 | −17.896 | 1.00 | 30.81 | N |
| ATOM | 402 | CA | VAL | A | 147 | 4.178 | 1.166 | −16.667 | 1.00 | 31.85 | C |
| ATOM | 403 | CB | VAL | A | 147 | 2.724 | 0.904 | −16.190 | 1.00 | 32.15 | C |
| ATOM | 404 | CG1 | VAL | A | 147 | 1.732 | 1.514 | −17.160 | 1.00 | 31.82 | C |
| ATOM | 405 | CG2 | VAL | A | 147 | 2.446 | −0.582 | −16.001 | 1.00 | 32.49 | C |
| ATOM | 406 | C | VAL | A | 147 | 5.155 | 0.841 | −15.532 | 1.00 | 32.16 | C |
| ATOM | 407 | O | VAL | A | 147 | 5.660 | −0.283 | −15.451 | 1.00 | 31.05 | O |
| ATOM | 408 | N | ASP | A | 148 | 5.404 | 1.820 | −14.667 | 1.00 | 33.57 | N |
| ATOM | 409 | CA | ASP | A | 148 | 6.146 | 1.593 | −13.417 | 1.00 | 34.91 | C |
| ATOM | 410 | CB | ASP | A | 148 | 6.670 | 2.914 | −12.824 | 1.00 | 35.85 | C |
| ATOM | 411 | CG | ASP | A | 148 | 7.638 | 3.646 | −13.747 | 1.00 | 36.55 | C |
| ATOM | 412 | OD1 | ASP | A | 148 | 8.330 | 2.966 | −14.524 | 1.00 | 36.32 | O |
| ATOM | 413 | OD2 | ASP | A | 148 | 7.741 | 4.887 | −13.645 | 1.00 | 38.55 | O |
| ATOM | 414 | C | ASP | A | 148 | 5.279 | 0.896 | −12.371 | 1.00 | 35.13 | C |
| ATOM | 415 | O | ASP | A | 148 | 5.777 | 0.049 | −11.624 | 1.00 | 35.08 | O |
| ATOM | 416 | N | SER | A | 149 | 3.994 | 1.257 | −12.318 | 1.00 | 35.46 | N |
| ATOM | 417 | CA | SER | A | 149 | 3.051 | 0.702 | −11.336 | 1.00 | 35.95 | C |
| ATOM | 418 | CB | SER | A | 149 | 2.704 | 1.751 | −10.274 | 1.00 | 35.98 | C |
| ATOM | 419 | OG | SER | A | 149 | 3.863 | 2.405 | −9.796 | 1.00 | 36.47 | O |
| ATOM | 420 | C | SER | A | 149 | 1.774 | 0.250 | −12.035 | 1.00 | 36.84 | C |
| ATOM | 421 | O | SER | A | 149 | 1.220 | 0.997 | −12.837 | 1.00 | 37.32 | O |
| ATOM | 422 | N | THR | A | 150 | 1.312 | −0.960 | −11.738 | 1.00 | 36.86 | N |
| ATOM | 423 | CA | THR | A | 150 | 0.083 | −1.490 | −12.320 | 1.00 | 37.13 | C |
| ATOM | 424 | CB | THR | A | 150 | −0.164 | −2.947 | −11.868 | 1.00 | 36.89 | C |
| ATOM | 425 | OG1 | THR | A | 150 | 0.989 | −3.738 | −12.152 | 1.00 | 36.06 | O |
| ATOM | 426 | CG2 | THR | A | 150 | −1.367 | −3.551 | −12.577 | 1.00 | 37.45 | C |
| ATOM | 427 | C | THR | A | 150 | −1.122 | −0.622 | −11.921 | 1.00 | 38.61 | C |
| ATOM | 428 | O | THR | A | 150 | −1.362 | −0.418 | −10.731 | 1.00 | 38.30 | O |
| ATOM | 429 | N | PRO | A | 151 | −1.868 | −0.085 | −12.912 | 1.00 | 40.11 | N |
| ATOM | 430 | CA | PRO | A | 151 | −3.086 | 0.654 | −12.565 | 1.00 | 40.67 | C |
| ATOM | 431 | CB | PRO | A | 151 | −3.420 | 1.451 | −13.834 | 1.00 | 41.17 | C |
| ATOM | 432 | CG | PRO | A | 151 | −2.596 | 0.888 | −14.926 | 1.00 | 41.42 | C |
| ATOM | 433 | CD | PRO | A | 151 | −1.591 | −0.065 | −14.354 | 1.00 | 40.58 | C |
| ATOM | 434 | C | PRO | A | 151 | −4.238 | −0.266 | −12.155 | 1.00 | 40.60 | C |
| ATOM | 435 | O | PRO | A | 151 | −4.188 | −1.462 | −12.447 | 1.00 | 42.90 | O |
| ATOM | 436 | N | PRO | A | 152 | −5.264 | 0.282 | −11.471 | 1.00 | 40.34 | N |
| ATOM | 437 | CA | PRO | A | 152 | −6.337 | −0.567 | −10.927 | 1.00 | 41.53 | C |
| ATOM | 438 | CB | PRO | A | 152 | −7.200 | 0.403 | −10.108 | 1.00 | 41.33 | C |
| ATOM | 439 | CG | PRO | A | 152 | −6.778 | 1.799 | −10.501 | 1.00 | 40.27 | C |
| ATOM | 440 | CD | PRO | A | 152 | −5.451 | 1.714 | −11.185 | 1.00 | 39.68 | C |
| ATOM | 441 | C | PRO | A | 152 | −7.183 | −1.249 | −12.004 | 1.00 | 43.94 | C |
| ATOM | 442 | O | PRO | A | 152 | −7.219 | −0.777 | −13.135 | 1.00 | 44.40 | O |
| ATOM | 443 | N | PRO | A | 153 | −7.860 | −2.370 | −11.654 | 1.00 | 43.48 | N |
| ATOM | 444 | CA | PRO | A | 153 | −8.777 | −3.022 | −12.619 | 1.00 | 41.05 | C |
| ATOM | 445 | CB | PRO | A | 153 | −9.378 | −4.172 | −11.813 | 1.00 | 43.42 | C |
| ATOM | 446 | CG | PRO | A | 153 | −8.309 | −4.514 | −10.821 | 1.00 | 44.44 | C |
| ATOM | 447 | CD | PRO | A | 153 | −7.708 | −3.173 | −10.425 | 1.00 | 44.17 | C |
| ATOM | 448 | C | PRO | A | 153 | −9.855 | −2.067 | −13.114 | 1.00 | 37.15 | C |
| ATOM | 449 | O | PRO | A | 153 | −10.228 | −1.141 | −12.400 | 1.00 | 36.33 | O |
| ATOM | 450 | N | GLY | A | 154 | −10.325 | −2.275 | −14.342 | 1.00 | 33.47 | N |
| ATOM | 451 | CA | GLY | A | 154 | −11.272 | −1.352 | −14.998 | 1.00 | 29.97 | C |
| ATOM | 452 | C | GLY | A | 154 | −10.608 | −0.180 | −15.720 | 1.00 | 28.01 | C |
| ATOM | 453 | O | GLY | A | 154 | −11.281 | 0.612 | −16.362 | 1.00 | 27.72 | O |
| ATOM | 454 | N | THR | A | 155 | −9.280 | −0.110 | −15.657 | 1.00 | 25.29 | N |
| ATOM | 455 | CA | THR | A | 155 | −8.474 | 0.791 | −16.480 | 1.00 | 24.07 | C |
| ATOM | 456 | CB | THR | A | 155 | −6.978 | 0.732 | −16.081 | 1.00 | 23.76 | C |
| ATOM | 457 | OG1 | THR | A | 155 | −6.828 | 1.187 | −14.729 | 1.00 | 24.22 | O |
| ATOM | 458 | CG2 | THR | A | 155 | −6.090 | 1.572 | −16.985 | 1.00 | 23.99 | C |
| ATOM | 459 | C | THR | A | 155 | −8.628 | 0.397 | −17.944 | 1.00 | 23.01 | C |
| ATOM | 460 | O | THR | A | 155 | −8.732 | −0.798 | −18.281 | 1.00 | 22.74 | O |
| ATOM | 461 | N | ARG | A | 156 | −8.638 | 1.418 | −18.810 | 1.00 | 21.80 | N |
| ATOM | 462 | CA | ARG | A | 156 | −8.845 | 1.242 | −20.246 | 1.00 | 21.38 | C |
| ATOM | 463 | CB | ARG | A | 156 | −10.246 | 1.741 | −20.623 | 1.00 | 20.62 | C |
| ATOM | 464 | CG | ARG | A | 156 | −11.323 | 0.667 | −20.551 | 1.00 | 20.69 | C |
| ATOM | 465 | CD | ARG | A | 156 | −12.647 | 1.166 | −20.003 | 1.00 | 20.58 | C |
| ATOM | 466 | NE | ARG | A | 156 | −13.333 | 2.146 | −20.846 | 1.00 | 20.43 | N |
| ATOM | 467 | CZ | ARG | A | 156 | −14.238 | 1.873 | −21.791 | 1.00 | 20.39 | C |
| ATOM | 468 | NH1 | ARG | A | 156 | −14.597 | 0.621 | −22.085 | 1.00 | 20.74 | N |
| ATOM | 469 | NH2 | ARG | A | 156 | −14.801 | 2.876 | −22.466 | 1.00 | 19.82 | N |
| ATOM | 470 | C | ARG | A | 156 | −7.763 | 1.946 | −21.061 | 1.00 | 21.65 | C |
| ATOM | 471 | O | ARG | A | 156 | −7.141 | 2.899 | −20.590 | 1.00 | 21.91 | O |
| ATOM | 472 | N | VAL | A | 157 | −7.576 | 1.469 | −22.298 | 1.00 | 21.02 | N |
| ATOM | 473 | CA | VAL | A | 157 | −6.570 | 1.974 | −23.225 | 1.00 | 20.37 | C |
| ATOM | 474 | CB | VAL | A | 157 | −5.506 | 0.902 | −23.545 | 1.00 | 20.18 | C |
| ATOM | 475 | OG1 | VAL | A | 157 | −4.318 | 1.518 | −24.271 | 1.00 | 20.13 | C |
| ATOM | 476 | CG2 | VAL | A | 157 | −5.046 | 0.211 | −22.261 | 1.00 | 20.50 | C |
| ATOM | 477 | C | VAL | A | 157 | −7.261 | 2.407 | −24.521 | 1.00 | 20.83 | C |
| ATOM | 478 | O | VAL | A | 157 | −7.819 | 1.575 | −25.228 | 1.00 | 22.05 | O |
| ATOM | 479 | N | ARG | A | 158 | −7.215 | 3.707 | −24.822 | 1.00 | 19.34 | N |
| ATOM | 480 | CA | ARG | A | 158 | −7.853 | 4.273 | −26.002 | 1.00 | 18.86 | C |
| ATOM | 481 | CB | ARG | A | 158 | −8.705 | 5.478 | −25.613 | 1.00 | 18.57 | C |
| ATOM | 482 | CG | ARG | A | 158 | −9.573 | 6.055 | −26.751 | 1.00 | 18.02 | C |
| ATOM | 483 | CD | ARG | A | 158 | −10.413 | 7.221 | −26.277 | 1.00 | 17.77 | C |
| ATOM | 484 | NE | ARG | A | 158 | −11.285 | 6.812 | −25.183 | 1.00 | 17.43 | N |
| ATOM | 485 | CZ | ARG | A | 158 | −12.473 | 6.218 | −25.301 | 1.00 | 17.89 | C |
| ATOM | 486 | NH1 | ARG | A | 158 | −13.023 | 5.985 | −26.494 | 1.00 | 18.23 | N |
| ATOM | 487 | NH2 | ARG | A | 158 | −13.133 | 5.873 | −24.199 | 1.00 | 18.41 | N |
| ATOM | 488 | C | ARG | A | 158 | −6.815 | 4.716 | −27.020 | 1.00 | 18.57 | C |
| ATOM | 489 | O | ARG | A | 158 | −5.791 | 5.266 | −26.651 | 1.00 | 17.79 | O |
| ATOM | 490 | N | ALA | A | 159 | −7.097 | 4.464 | −28.297 | 1.00 | 18.77 | N |
| ATOM | 491 | CA | ALA | A | 159 | −6.292 | 4.980 | −29.404 | 1.00 | 18.35 | C |
| ATOM | 492 | CB | ALA | A | 159 | −5.718 | 3.828 | −30.223 | 1.00 | 18.12 | C |
| ATOM | 493 | C | ALA | A | 159 | −7.178 | 5.872 | −30.265 | 1.00 | 18.06 | C |
| ATOM | 494 | O | ALA | A | 159 | −8.353 | 5.566 | −30.492 | 1.00 | 17.72 | O |
| ATOM | 495 | N | MET | A | 160 | −6.616 | 6.985 | −30.727 | 1.00 | 18.46 | N |
| ATOM | 496 | CA | MET | A | 160 | −7.357 | 7.961 | −31.562 | 1.00 | 18.39 | C |
| ATOM | 497 | CB | MET | A | 160 | −8.022 | 9.033 | −30.684 | 1.00 | 18.38 | C |
| ATOM | 498 | CG | MET | A | 160 | −8.649 | 10.203 | −31.426 | 1.00 | 18.89 | C |
| ATOM | 499 | SD | MET | A | 160 | −9.357 | 11.528 | −30.411 | 1.00 | 19.48 | S |
| ATOM | 500 | CE | MET | A | 160 | −10.535 | 10.672 | −29.374 | 1.00 | 19.31 | C |
| ATOM | 501 | C | MET | A | 160 | −6.364 | 8.584 | −32.541 | 1.00 | 17.73 | C |
| ATOM | 502 | O | MET | A | 160 | −5.230 | 8.882 | −32.166 | 1.00 | 17.06 | O |
| ATOM | 503 | N | ALA | A | 161 | −6.806 | 8.789 | −33.782 | 1.00 | 17.42 | N |
| ATOM | 504 | CA | ALA | A | 161 | −5.993 | 9.461 | −34.795 | 1.00 | 17.86 | C |
| ATOM | 505 | CB | ALA | A | 161 | −6.113 | 8.744 | −36.121 | 1.00 | 17.68 | C |
| ATOM | 506 | C | ALA | A | 161 | −6.391 | 10.940 | −34.946 | 1.00 | 18.20 | C |
| ATOM | 507 | O | ALA | A | 161 | −7.562 | 11.302 | −34.810 | 1.00 | 17.67 | O |
| ATOM | 508 | N | ILE | A | 162 | −5.392 | 11.784 | −35.208 | 1.00 | 18.67 | N |
| ATOM | 509 | CA | ILE | A | 162 | −5.609 | 13.177 | −35.642 | 1.00 | 19.84 | C |
| ATOM | 510 | CB | ILE | A | 162 | −5.519 | 14.199 | −34.484 | 1.00 | 19.92 | C |
| ATOM | 511 | CG1 | ILE | A | 162 | −4.127 | 14.224 | −33.844 | 1.00 | 20.25 | C |
| ATOM | 512 | CD1 | ILE | A | 162 | −3.883 | 15.396 | −32.923 | 1.00 | 20.51 | C |
| ATOM | 513 | CG2 | ILE | A | 162 | −6.547 | 13.898 | −33.407 | 1.00 | 20.18 | C |
| ATOM | 514 | C | ILE | A | 162 | −4.588 | 13.540 | −36.712 | 1.00 | 21.08 | C |
| ATOM | 515 | O | ILE | A | 162 | −3.515 | 12.940 | −36.770 | 1.00 | 21.83 | O |
| ATOM | 516 | N | TYR | A | 163 | −4.908 | 14.524 | −37.542 | 1.00 | 22.55 | N |
| ATOM | 517 | CA | TYR | A | 163 | −3.959 | 15.008 | −38.558 | 1.00 | 24.03 | C |
| ATOM | 518 | CB | TYR | A | 163 | −4.701 | 15.763 | −39.661 | 1.00 | 23.37 | C |
| ATOM | 519 | CG | TYR | A | 163 | −5.622 | 14.873 | −40.464 | 1.00 | 22.84 | C |
| ATOM | 520 | CD1 | TYR | A | 163 | −5.109 | 13.825 | −41.217 | 1.00 | 22.99 | C |
| ATOM | 521 | CE1 | TYR | A | 163 | −5.939 | 12.988 | −41.955 | 1.00 | 22.63 | C |
| ATOM | 522 | CZ | TYR | A | 163 | −7.299 | 13.201 | −41.947 | 1.00 | 22.54 | C |
| ATOM | 523 | OH | TYR | A | 163 | −8.101 | 12.353 | −42.675 | 1.00 | 23.34 | O |
| ATOM | 524 | CE2 | TYR | A | 163 | −7.844 | 14.245 | −41.197 | 1.00 | 22.74 | C |
| ATOM | 525 | CD2 | TYR | A | 163 | −7.010 | 15.066 | −40.465 | 1.00 | 22.96 | C |
| ATOM | 526 | C | TYR | A | 163 | −2.911 | 15.905 | −37.905 | 1.00 | 25.64 | C |
| ATOM | 527 | O | TYR | A | 163 | −3.229 | 16.686 | −37.003 | 1.00 | 25.94 | O |
| ATOM | 528 | N | LYS | A | 164 | −1.667 | 15.779 | −38.351 | 1.00 | 27.86 | N |
| ATOM | 529 | CA | LYS | A | 164 | −0.539 | 16.517 | −37.761 | 1.00 | 30.27 | C |
| ATOM | 530 | CB | LYS | A | 164 | 0.798 | 15.831 | −38.061 | 1.00 | 31.39 | C |
| ATOM | 531 | CG | LYS | A | 164 | 1.939 | 16.378 | −37.190 | 1.00 | 32.25 | C |
| ATOM | 532 | CD | LYS | A | 164 | 3.261 | 15.643 | −37.334 | 1.00 | 33.35 | C |
| ATOM | 533 | CE | LYS | A | 164 | 4.056 | 16.140 | −38.537 | 1.00 | 33.82 | C |
| ATOM | 534 | NZ | LYS | A | 164 | 5.493 | 15.752 | −38.490 | 1.00 | 33.22 | N |
| ATOM | 535 | C | LYS | A | 164 | −0.493 | 17.975 | −38.227 | 1.00 | 31.70 | C |
| ATOM | 536 | O | LYS | A | 164 | −0.196 | 18.867 | −37.427 | 1.00 | 33.94 | O |
| ATOM | 537 | N | GLN | A | 165 | −0.790 | 18.223 | −39.499 | 1.00 | 32.37 | N |
| ATOM | 538 | CA | GLN | A | 165 | −0.698 | 19.563 | −40.076 | 1.00 | 33.96 | C |
| ATOM | 539 | CB | GLN | A | 165 | −0.806 | 19.501 | −41.600 | 1.00 | 35.04 | C |
| ATOM | 540 | CG | GLN | A | 165 | 0.342 | 18.751 | −42.266 | 1.00 | 35.36 | C |
| ATOM | 541 | CD | GLN | A | 165 | 0.210 | 18.678 | −43.774 | 1.00 | 35.69 | C |
| ATOM | 542 | OE1 | GLN | A | 165 | −0.854 | 18.944 | −44.367 | 1.00 | 37.44 | O |
| ATOM | 543 | NE2 | GLN | A | 165 | 1.316 | 18.340 | −44.418 | 1.00 | 36.82 | N |
| ATOM | 544 | C | GLN | A | 165 | −1.770 | 20.507 | −39.518 | 1.00 | 34.38 | C |
| ATOM | 545 | O | GLN | A | 165 | −2.965 | 20.171 | −39.500 | 1.00 | 35.83 | O |
| ATOM | 546 | N | SER | A | 166 | −1.329 | 21.691 | −39.082 | 1.00 | 34.94 | N |
| ATOM | 547 | CA | SER | A | 166 | −2.187 | 22.653 | −38.374 | 1.00 | 35.17 | C |
| ATOM | 548 | CB | SER | A | 166 | −1.387 | 23.911 | −37.998 | 1.00 | 35.01 | C |
| ATOM | 549 | OG | SER | A | 166 | −0.661 | 24.417 | −39.107 | 1.00 | 35.37 | O |
| ATOM | 550 | C | SER | A | 166 | −3.455 | 23.046 | −39.139 | 1.00 | 35.29 | C |
| ATOM | 551 | O | SER | A | 166 | −4.504 | 23.262 | −38.523 | 1.00 | 34.26 | O |
| ATOM | 552 | N | GLN | A | 167 | −3.360 | 23.128 | −40.466 | 1.00 | 35.40 | N |
| ATOM | 553 | CA | GLN | A | 167 | −4.518 | 23.460 | −41.306 | 1.00 | 36.40 | C |
| ATOM | 554 | CB | GLN | A | 167 | −4.078 | 23.948 | −42.705 | 1.00 | 38.54 | C |
| ATOM | 555 | CG | GLN | A | 167 | −3.538 | 22.892 | −43.665 | 1.00 | 39.85 | C |
| ATOM | 556 | CD | GLN | A | 167 | −2.056 | 22.598 | −43.494 | 1.00 | 42.19 | C |
| ATOM | 557 | OE1 | GLN | A | 167 | −1.444 | 22.924 | −42.466 | 1.00 | 40.05 | O |
| ATOM | 558 | NE2 | GLN | A | 167 | −1.470 | 21.958 | −44.509 | 1.00 | 43.10 | N |
| ATOM | 559 | C | GLN | A | 167 | −5.576 | 22.350 | −41.428 | 1.00 | 35.37 | C |
| ATOM | 560 | O | GLN | A | 167 | −6.711 | 22.631 | −41.830 | 1.00 | 35.09 | O |
| ATOM | 561 | N | HIS | A | 168 | −5.203 | 21.106 | −41.111 | 1.00 | 34.37 | N |
| ATOM | 562 | CA | HIS | A | 168 | −6.130 | 19.962 | −41.127 | 1.00 | 32.72 | C |
| ATOM | 563 | CB | HIS | A | 168 | −5.549 | 18.809 | −41.947 | 1.00 | 32.21 | C |
| ATOM | 564 | CG | HIS | A | 168 | −5.344 | 19.132 | −43.391 | 1.00 | 31.62 | C |
| ATOM | 565 | ND1 | HIS | A | 168 | −6.372 | 19.540 | −44.213 | 1.00 | 31.67 | N |
| ATOM | 566 | CE1 | HIS | A | 168 | −5.895 | 19.771 | −45.424 | 1.00 | 32.38 | C |
| ATOM | 567 | NE2 | HIS | A | 168 | −4.593 | 19.539 | −45.411 | 1.00 | 32.52 | N |
| ATOM | 568 | CD2 | HIS | A | 168 | −4.223 | 19.155 | −44.144 | 1.00 | 32.16 | C |
| ATOM | 569 | C | HIS | A | 168 | −6.529 | 19.433 | −39.731 | 1.00 | 32.59 | C |
| ATOM | 570 | O | HIS | A | 168 | −7.380 | 18.556 | −39.652 | 1.00 | 32.16 | O |
| ATOM | 571 | N | MET | A | 169 | −5.925 | 19.942 | −38.661 | 1.00 | 32.88 | N |
| ATOM | 572 | CA | MET | A | 169 | −6.062 | 19.345 | −37.313 | 1.00 | 34.44 | C |
| ATOM | 573 | CB | MET | A | 169 | −5.193 | 20.093 | −36.282 | 1.00 | 38.23 | C |
| ATOM | 574 | CG | MET | A | 169 | −5.000 | 19.302 | −34.996 | 1.00 | 42.56 | C |
| ATOM | 575 | SD | MET | A | 169 | −3.561 | 19.742 | −34.007 | 1.00 | 51.64 | S |
| ATOM | 576 | CE | MET | A | 169 | −2.407 | 18.596 | −34.728 | 1.00 | 50.03 | C |
| ATOM | 577 | C | MET | A | 169 | −7.504 | 19.219 | −36.781 | 1.00 | 32.93 | C |
| ATOM | 578 | O | MET | A | 169 | −7.804 | 18.316 | −35.992 | 1.00 | 31.69 | O |
| ATOM | 579 | N | THR | A | 170 | −8.385 | 20.128 | −37.204 | 1.00 | 30.87 | N |
| ATOM | 580 | CA | THR | A | 170 | −9.787 | 20.113 | −36.766 | 1.00 | 29.94 | C |
| ATOM | 581 | CB | THR | A | 170 | −10.436 | 21.501 | −36.851 | 1.00 | 29.47 | C |
| ATOM | 582 | OG1 | THR | A | 170 | −10.452 | 21.945 | −38.214 | 1.00 | 29.19 | O |
| ATOM | 583 | CG2 | THR | A | 170 | −9.666 | 22.484 | −35.961 | 1.00 | 29.84 | C |
| ATOM | 584 | C | THR | A | 170 | −10.661 | 19.104 | −37.518 | 1.00 | 29.75 | C |
| ATOM | 585 | O | THR | A | 170 | −11.752 | 18.770 | −37.039 | 1.00 | 29.47 | O |
| ATOM | 586 | N | GLU | A | 171 | −10.199 | 18.628 | −38.673 | 1.00 | 29.42 | N |
| ATOM | 587 | CA | GLU | A | 171 | −10.925 | 17.600 | −39.425 | 1.00 | 29.97 | C |
| ATOM | 588 | CB | GLU | A | 171 | −10.398 | 17.472 | −40.858 | 1.00 | 33.24 | C |
| ATOM | 589 | CG | GLU | A | 171 | −10.514 | 18.748 | −41.687 | 1.00 | 35.31 | C |
| ATOM | 590 | CD | GLU | A | 171 | −10.233 | 18.486 | −43.154 | 1.00 | 38.93 | C |
| ATOM | 591 | OE1 | GLU | A | 171 | −9.251 | 19.039 | −43.711 | 1.00 | 42.47 | O |
| ATOM | 592 | OE2 | GLU | A | 171 | −11.000 | 17.692 | −43.742 | 1.00 | 43.26 | O |
| ATOM | 593 | C | GLU | A | 171 | −10.815 | 16.237 | −38.747 | 1.00 | 27.56 | C |
| ATOM | 594 | O | GLU | A | 171 | −9.760 | 15.869 | −38.214 | 1.00 | 26.38 | O |
| ATOM | 595 | N | VAL | A | 172 | −11.901 | 15.481 | −38.804 | 1.00 | 25.98 | N |
| ATOM | 596 | CA | VAL | A | 172 | −11.967 | 14.153 | −38.208 | 1.00 | 25.78 | C |
| ATOM | 597 | CB | VAL | A | 172 | −13.436 | 13.678 | −38.047 | 1.00 | 25.30 | C |
| ATOM | 598 | CG1 | VAL | A | 172 | −13.499 | 12.232 | −37.540 | 1.00 | 24.83 | C |
| ATOM | 599 | CG2 | VAL | A | 172 | −14.207 | 14.647 | −37.145 | 1.00 | 25.13 | C |
| ATOM | 600 | C | VAL | A | 172 | −11.242 | 13.175 | −39.128 | 1.00 | 24.83 | C |
| ATOM | 601 | O | VAL | A | 172 | −11.544 | 13.096 | −40.332 | 1.00 | 24.48 | O |
| ATOM | 602 | N | VAL | A | 173 | −10.285 | 12.445 | −38.558 | 1.00 | 24.28 | N |
| ATOM | 603 | CA | VAL | A | 173 | −9.601 | 11.384 | −39.287 | 1.00 | 25.13 | C |
| ATOM | 604 | CB | VAL | A | 173 | −8.312 | 10.904 | −38.585 | 1.00 | 25.06 | C |
| ATOM | 605 | CG1 | VAL | A | 173 | −7.704 | 9.716 | −39.329 | 1.00 | 25.06 | C |
| ATOM | 606 | CG2 | VAL | A | 173 | −7.289 | 12.029 | −38.497 | 1.00 | 25.50 | C |
| ATOM | 607 | C | VAL | A | 173 | −10.561 | 10.220 | −39.466 | 1.00 | 24.71 | C |
| ATOM | 608 | O | VAL | A | 173 | −10.956 | 9.607 | −38.487 | 1.00 | 23.43 | O |
| ATOM | 609 | N | ARG | A | 174 | −10.962 | 9.970 | −40.701 | 1.00 | 25.23 | N |
| ATOM | 610 | CA | ARG | A | 174 | −11.845 | 8.872 | −41.046 | 1.00 | 26.47 | C |
| ATOM | 611 | CB | ARG | A | 174 | −13.290 | 9.317 | −41.333 | 1.00 | 28.99 | C |
| ATOM | 612 | CG | ARG | A | 174 | −13.423 | 10.476 | −42.302 | 1.00 | 31.82 | C |
| ATOM | 613 | CD | ARG | A | 174 | −14.884 | 10.758 | −42.680 | 1.00 | 34.23 | C |
| ATOM | 614 | NE | ARG | A | 174 | −15.552 | 11.631 | −41.698 | 1.00 | 37.41 | N |
| ATOM | 615 | CZ | ARG | A | 174 | −16.320 | 11.239 | −40.673 | 1.00 | 39.75 | C |
| ATOM | 616 | NH1 | ARG | A | 174 | −16.576 | 9.950 | −40.439 | 1.00 | 40.33 | N |
| ATOM | 617 | NH2 | ARG | A | 174 | −16.819 | 12.167 | −39.854 | 1.00 | 39.91 | N |
| ATOM | 618 | C | ARG | A | 174 | −11.275 | 8.176 | −42.268 | 1.00 | 25.27 | C |
| ATOM | 619 | O | ARG | A | 174 | −10.398 | 8.711 | −42.963 | 1.00 | 23.64 | O |
| ATOM | 620 | N | ARG | A | 175 | −11.780 | 6.968 | −42.531 | 1.00 | 23.29 | N |
| ATOM | 621 | CA | ARG | A | 175 | −11.439 | 6.231 | −43.724 | 1.00 | 22.83 | C |
| ATOM | 622 | CB | ARG | A | 175 | −11.996 | 4.808 | −43.619 | 1.00 | 22.45 | C |
| ATOM | 623 | CG | ARG | A | 175 | −11.190 | 3.888 | −42.716 | 1.00 | 21.60 | C |
| ATOM | 624 | CD | ARG | A | 175 | −10.921 | 2.522 | −43.309 | 1.00 | 21.47 | C |
| ATOM | 625 | NE | ARG | A | 175 | −12.047 | 1.608 | −43.262 | 1.00 | 20.91 | N |
| ATOM | 626 | CZ | ARG | A | 175 | −12.058 | 0.379 | −43.803 | 1.00 | 20.16 | C |
| ATOM | 627 | NH1 | ARG | A | 175 | −13.168 | −0.355 | −43.705 | 1.00 | 19.50 | N |
| ATOM | 628 | NH2 | ARG | A | 175 | −11.011 | −0.111 | −44.483 | 1.00 | 20.11 | N |
| ATOM | 629 | C | ARG | A | 175 | −11.958 | 6.924 | −44.986 | 1.00 | 22.31 | C |
| ATOM | 630 | O | ARG | A | 175 | −12.969 | 7.608 | −44.937 | 1.00 | 21.70 | O |
| ATOM | 631 | N | CYS | A | 176 | −11.253 | 6.744 | −46.110 | 1.00 | 21.81 | N |
| ATOM | 632 | CA | CYS | A | 176 | −11.636 | 7.387 | −47.363 | 1.00 | 21.70 | C |
| ATOM | 633 | CB | CYS | A | 176 | −10.492 | 7.307 | −48.379 | 1.00 | 21.82 | C |
| ATOM | 634 | SG | CYS | A | 176 | −10.186 | 5.663 | −49.042 | 1.00 | 22.63 | S |
| ATOM | 635 | C | CYS | A | 176 | −12.880 | 6.659 | −47.888 | 1.00 | 21.49 | C |
| ATOM | 636 | O | CYS | A | 176 | −13.079 | 5.506 | −47.563 | 1.00 | 20.33 | O |
| ATOM | 637 | N | PRO | A | 177 | −13.639 | 7.290 | −48.795 | 1.00 | 21.19 | N |
| ATOM | 638 | CA | PRO | A | 177 | −14.822 | 6.635 | −49.367 | 1.00 | 21.19 | C |
| ATOM | 639 | CB | PRO | A | 177 | −15.230 | 7.585 | −50.497 | 1.00 | 21.96 | C |
| ATOM | 640 | CG | PRO | A | 177 | −14.752 | 8.922 | −50.049 | 1.00 | 21.76 | C |
| ATOM | 641 | CD | PRO | A | 177 | −13.437 | 8.637 | −49.379 | 1.00 | 21.49 | C |
| ATOM | 642 | C | PRO | A | 177 | −14.617 | 5.216 | −49.923 | 1.00 | 21.40 | C |
| ATOM | 643 | O | PRO | A | 177 | −15.443 | 4.344 | −49.683 | 1.00 | 20.89 | O |
| ATOM | 644 | N | HIS | A | 178 | −13.505 | 4.986 | −50.616 | 1.00 | 21.28 | N |
| ATOM | 645 | CA | HIS | A | 178 | −13.192 | 3.645 | −51.117 | 1.00 | 20.90 | C |
| ATOM | 646 | CB | HIS | A | 178 | −11.950 | 3.650 | −52.034 | 1.00 | 20.75 | C |
| ATOM | 647 | CG | HIS | A | 178 | −11.268 | 2.323 | −52.123 | 1.00 | 20.31 | C |
| ATOM | 648 | ND1 | HIS | A | 178 | −11.847 | 1.229 | −52.731 | 1.00 | 19.80 | N |
| ATOM | 649 | CE1 | HIS | A | 178 | −11.021 | 0.201 | −52.659 | 1.00 | 20.56 | C |
| ATOM | 650 | NE2 | HIS | A | 178 | −9.938 | 0.580 | −51.999 | 1.00 | 20.73 | N |
| ATOM | 651 | CD2 | HIS | A | 178 | −10.070 | 1.906 | −51.655 | 1.00 | 20.36 | C |
| ATOM | 652 | C | HIS | A | 178 | −13.016 | 2.629 | −49.992 | 1.00 | 21.35 | C |
| ATOM | 653 | O | HIS | A | 178 | −13.608 | 1.548 | −50.036 | 1.00 | 21.06 | O |
| ATOM | 654 | N | HIS | A | 179 | −12.186 | 2.960 | −49.004 | 1.00 | 21.99 | N |
| ATOM | 655 | CA | HIS | A | 179 | −11.918 | 2.033 | −47.896 | 1.00 | 22.64 | C |
| ATOM | 656 | CB | HIS | A | 179 | −10.674 | 2.442 | −47.086 | 1.00 | 22.06 | C |
| ATOM | 657 | CG | HIS | A | 179 | −9.385 | 1.956 | −47.676 | 1.00 | 21.42 | C |
| ATOM | 658 | ND1 | HIS | A | 179 | −8.376 | 2.800 | −48.095 | 1.00 | 20.74 | N |
| ATOM | 659 | CE1 | HIS | A | 179 | −7.378 | 2.085 | −48.577 | 1.00 | 21.06 | C |
| ATOM | 660 | NE2 | HIS | A | 179 | −7.697 | 0.808 | −48.480 | 1.00 | 21.08 | N |
| ATOM | 661 | CD2 | HIS | A | 179 | −8.946 | 0.700 | −47.921 | 1.00 | 21.22 | C |
| ATOM | 662 | C | HIS | A | 179 | −13.150 | 1.841 | −46.994 | 1.00 | 23.30 | C |
| ATOM | 663 | O | HIS | A | 179 | −13.437 | 0.730 | −46.558 | 1.00 | 24.60 | O |
| ATOM | 664 | N | GLU | A | 180 | −13.694 | 2.907 | −46.764 | 1.00 | 24.97 | N |
| ATOM | 665 | CA | GLU | A | 180 | −15.188 | 2.821 | −46.084 | 1.00 | 28.45 | C |
| ATOM | 666 | CB | GLU | A | 180 | −15.866 | 4.191 | −46.063 | 1.00 | 28.61 | C |
| ATOM | 667 | CG | GLU | A | 180 | −17.214 | 4.211 | −45.351 | 1.00 | 29.76 | C |
| ATOM | 668 | CD | GLU | A | 180 | −17.780 | 5.611 | −45.166 | 1.00 | 31.32 | C |
| ATOM | 669 | OE1 | GLU | A | 180 | −17.036 | 6.610 | −45.322 | 1.00 | 30.62 | O |
| ATOM | 670 | OE2 | GLU | A | 180 | −18.985 | 5.708 | −44.834 | 1.00 | 34.95 | O |
| ATOM | 671 | C | GLU | A | 180 | −16.142 | 1.779 | −46.698 | 1.00 | 30.91 | C |
| ATOM | 672 | O | GLU | A | 180 | −16.855 | 1.095 | −45.962 | 1.00 | 32.85 | O |
| ATOM | 673 | N | ARG | A | 181 | −16.152 | 1.668 | −48.028 | 1.00 | 33.54 | N |
| ATOM | 674 | CA | ARG | A | 181 | −17.047 | 0.742 | −48.733 | 1.00 | 34.92 | C |
| ATOM | 675 | CB | ARG | A | 181 | −17.545 | 1.389 | −50.037 | 1.00 | 37.46 | C |
| ATOM | 676 | CG | ARG | A | 181 | −18.400 | 2.630 | −49.786 | 1.00 | 41.29 | C |
| ATOM | 677 | CD | ARG | A | 181 | −19.001 | 3.218 | −51.064 | 1.00 | 44.07 | C |
| ATOM | 678 | NE | ARG | A | 181 | −20.174 | 4.065 | −50.794 | 1.00 | 47.57 | N |
| ATOM | 679 | CZ | ARG | A | 181 | −21.389 | 3.622 | −50.445 | 1.00 | 49.10 | C |
| ATOM | 680 | NH1 | ARG | A | 181 | −21.649 | 2.317 | −50.318 | 1.00 | 49.66 | N |
| ATOM | 681 | NH2 | ARG | A | 181 | −22.366 | 4.498 | −50.218 | 1.00 | 49.89 | N |
| ATOM | 682 | C | ARG | A | 181 | −16.450 | −0.651 | −49.006 | 1.00 | 33.63 | C |
| ATOM | 683 | O | ARG | A | 181 | −17.139 | −1.503 | −49.559 | 1.00 | 34.73 | O |
| ATOM | 684 | N | CYS | A | 182 | −15.194 | −0.883 | −48.610 | 1.00 | 32.02 | N |
| ATOM | 685 | CA | CYS | A | 182 | −14.529 | −2.180 | −48.818 | 1.00 | 31.47 | C |
| ATOM | 686 | CB | CYS | A | 182 | −13.034 | −2.117 | −48.485 | 1.00 | 31.67 | C |
| ATOM | 687 | SG | CYS | A | 182 | −11.988 | −1.394 | −49.769 | 1.00 | 29.55 | S |
| ATOM | 688 | C | CYS | A | 182 | −15.129 | −3.283 | −47.970 | 1.00 | 32.29 | C |
| ATOM | 689 | O | CYS | A | 182 | −15.628 | −3.033 | −46.879 | 1.00 | 32.51 | O |
| ATOM | 690 | N | SER | A | 183 | −15.056 | −4.510 | −48.484 | 1.00 | 33.19 | N |
| ATOM | 691 | CA | SER | A | 183 | −15.533 | −5.694 | −47.778 | 1.00 | 32.22 | C |
| ATOM | 692 | CB | SER | A | 183 | −16.057 | −6.740 | −48.768 | 1.00 | 32.47 | C |
| ATOM | 693 | OG | SER | A | 183 | −17.187 | −6.239 | −49.455 | 1.00 | 33.63 | O |
| ATOM | 694 | C | SER | A | 183 | −14.397 | −6.255 | −46.936 | 1.00 | 30.84 | C |
| ATOM | 695 | O | SER | A | 183 | −13.865 | −7.331 | −47.217 | 1.00 | 31.02 | O |
| ATOM | 696 | N | ASP | A | 184 | −14.035 | −5.511 | −45.894 | 1.00 | 29.27 | N |
| ATOM | 697 | CA | ASP | A | 184 | −12.933 | −5.882 | −45.006 | 1.00 | 26.83 | C |
| ATOM | 698 | CB | ASP | A | 184 | −11.710 | −4.996 | −45.283 | 1.00 | 26.08 | C |
| ATOM | 699 | CG | ASP | A | 184 | −11.976 | −3.491 | −45.047 | 1.00 | 25.54 | C |
| ATOM | 700 | OD1 | ASP | A | 184 | −12.938 | −3.108 | −44.347 | 1.00 | 23.90 | O |
| ATOM | 701 | OD2 | ASP | A | 184 | −11.174 | −2.686 | −45.554 | 1.00 | 25.57 | O |
| ATOM | 702 | C | ASP | A | 184 | −13.351 | −5.811 | −43.541 | 1.00 | 26.80 | C |
| ATOM | 703 | O | ASP | A | 184 | −12.509 | −5.645 | −42.658 | 1.00 | 27.86 | O |
| ATOM | 704 | N | SER | A | 185 | −14.648 | −5.949 | −43.279 | 1.00 | 26.14 | N |
| ATOM | 705 | CA | SER | A | 185 | −15.168 | −5.937 | −41.916 | 1.00 | 26.05 | C |
| ATOM | 706 | CB | SER | A | 185 | −16.701 | −5.879 | −41.931 | 1.00 | 26.35 | C |
| ATOM | 707 | OG | SER | A | 185 | −17.222 | −5.862 | −40.611 | 1.00 | 26.75 | O |
| ATOM | 708 | C | SER | A | 185 | −14.715 | −7.170 | −41.121 | 1.00 | 26.16 | C |
| ATOM | 709 | O | SER | A | 185 | −14.549 | −8.254 | −41.677 | 1.00 | 25.12 | O |
| ATOM | 710 | N | ASP | A | 186 | −14.521 | −6.967 | −39.816 | 1.00 | 26.20 | N |
| ATOM | 711 | CA | ASP | A | 186 | −14.262 | −8.044 | −38.851 | 1.00 | 25.19 | C |
| ATOM | 712 | CB | ASP | A | 186 | −13.005 | −7.732 | −38.009 | 1.00 | 25.78 | C |
| ATOM | 713 | CG | ASP | A | 186 | −13.106 | −6.427 | −37.196 | 1.00 | 26.30 | C |
| ATOM | 714 | OD1 | ASP | A | 186 | −14.106 | −5.692 | −37.269 | 1.00 | 26.55 | O |
| ATOM | 715 | OD2 | ASP | A | 186 | −12.115 | −6.100 | −36.511 | 1.00 | 27.88 | O |
| ATOM | 716 | C | ASP | A | 186 | −15.489 | −8.336 | −37.955 | 1.00 | 24.36 | C |
| ATOM | 717 | O | ASP | A | 186 | −15.387 | −9.052 | −36.961 | 1.00 | 24.76 | O |
| ATOM | 718 | N | GLY | A | 187 | −16.645 | −7.772 | −38.298 | 1.00 | 23.91 | N |
| ATOM | 719 | CA | GLY | A | 187 | −17.855 | −7.913 | −37.487 | 1.00 | 23.24 | C |
| ATOM | 720 | C | GLY | A | 187 | −17.933 | −7.011 | −36.258 | 1.00 | 23.10 | C |
| ATOM | 721 | O | GLY | A | 187 | −19.013 | −6.834 | −35.705 | 1.00 | 22.12 | O |
| ATOM | 722 | N | LEU | A | 188 | −16.804 | −6.446 | −35.824 | 1.00 | 23.43 | N |
| ATOM | 723 | CA | LEU | A | 188 | −16.766 | −5.584 | −34.640 | 1.00 | 23.91 | C |
| ATOM | 724 | CB | LEU | A | 188 | −15.666 | −6.040 | −33.682 | 1.00 | 23.72 | C |
| ATOM | 725 | CG | LEU | A | 188 | −15.746 | −7.485 | −33.180 | 1.00 | 23.62 | C |
| ATOM | 726 | CD1 | LEU | A | 188 | −14.433 | −7.922 | −32.548 | 1.00 | 23.77 | C |
| ATOM | 727 | CD2 | LEU | A | 188 | −16.898 | −7.649 | −32.201 | 1.00 | 24.21 | C |
| ATOM | 728 | C | LEU | A | 188 | −16.560 | −4.108 | −35.008 | 1.00 | 24.17 | C |
| ATOM | 729 | O | LEU | A | 188 | −17.223 | −3.245 | −34.446 | 1.00 | 25.95 | O |
| ATOM | 730 | N | ALA | A | 189 | −15.657 | −3.821 | −35.949 | 1.00 | 23.29 | N |
| ATOM | 731 | CA | ALA | A | 189 | −15.240 | −2.445 | −36.210 | 1.00 | 23.03 | C |
| ATOM | 732 | CB | ALA | A | 189 | −13.826 | −2.400 | −36.774 | 1.00 | 23.44 | C |
| ATOM | 733 | C | ALA | A | 189 | −16.197 | −1.703 | −37.136 | 1.00 | 22.74 | C |
| ATOM | 734 | O | ALA | A | 189 | −16.658 | −2.259 | −38.129 | 1.00 | 22.87 | O |
| ATOM | 735 | N | PRO | A | 190 | −16.486 | −0.420 | −36.828 | 1.00 | 22.53 | N |
| ATOM | 736 | CA | PRO | A | 190 | −17.238 | 0.423 | −37.766 | 1.00 | 22.16 | C |
| ATOM | 737 | CB | PRO | A | 190 | −17.366 | 1.751 | −37.035 | 1.00 | 22.35 | C |
| ATOM | 738 | CG | PRO | A | 190 | −17.111 | 1.460 | −35.609 | 1.00 | 22.21 | C |
| ATOM | 739 | CD | PRO | A | 190 | −16.227 | 0.262 | −35.551 | 1.00 | 22.26 | C |
| ATOM | 740 | C | PRO | A | 190 | −16.482 | 0.629 | −39.081 | 1.00 | 22.08 | C |
| ATOM | 741 | O | PRO | A | 190 | −15.255 | 0.717 | −39.059 | 1.00 | 21.72 | O |
| ATOM | 742 | N | PRO | A | 191 | −17.206 | 0.707 | −40.212 | 1.00 | 22.21 | N |
| ATOM | 743 | CA | PRO | A | 191 | −16.538 | 0.839 | −41.509 | 1.00 | 22.01 | C |
| ATOM | 744 | CB | PRO | A | 191 | −17.684 | 0.664 | −42.528 | 1.00 | 22.30 | C |
| ATOM | 745 | CG | PRO | A | 191 | −18.950 | 0.960 | −41.771 | 1.00 | 22.30 | C |
| ATOM | 746 | CD | PRO | A | 191 | −18.670 | 0.579 | −40.348 | 1.00 | 22.20 | C |
| ATOM | 747 | C | PRO | A | 191 | −15.785 | 2.155 | −41.747 | 1.00 | 22.38 | C |
| ATOM | 748 | O | PRO | A | 191 | −14.893 | 2.178 | −42.591 | 1.00 | 23.31 | O |
| ATOM | 749 | N | GLN | A | 192 | −16.134 | 3.224 | −41.027 | 1.00 | 22.37 | N |
| ATOM | 750 | CA | GLN | A | 192 | −15.442 | 4.523 | −41.173 | 1.00 | 22.68 | C |
| ATOM | 751 | CB | GLN | A | 192 | −16.353 | 5.676 | −40.770 | 1.00 | 24.27 | C |
| ATOM | 752 | CG | GLN | A | 192 | −17.558 | 5.896 | −41.655 | 1.00 | 26.00 | C |
| ATOM | 753 | CD | GLN | A | 192 | −18.331 | 7.159 | −41.298 | 1.00 | 28.64 | C |
| ATOM | 754 | OE1 | GLN | A | 192 | −18.268 | 7.664 | −40.165 | 1.00 | 30.15 | O |
| ATOM | 755 | NE2 | GLN | A | 192 | −19.071 | 7.694 | −42.277 | 1.00 | 28.37 | N |
| ATOM | 756 | C | GLN | A | 192 | −14.107 | 4.655 | −40.393 | 1.00 | 20.92 | C |
| ATOM | 757 | O | GLN | A | 192 | −13.337 | 5.559 | −40.698 | 1.00 | 19.82 | O |
| ATOM | 758 | N | HIS | A | 193 | −13.867 | 3.788 | −39.398 | 1.00 | 19.23 | N |
| ATOM | 759 | CA | HIS | A | 193 | −12.728 | 3.962 | −38.476 | 1.00 | 18.32 | C |
| ATOM | 760 | CB | HIS | A | 193 | −12.911 | 3.168 | −37.165 | 1.00 | 17.79 | C |
| ATOM | 761 | CG | HIS | A | 193 | −13.907 | 3.767 | −36.223 | 1.00 | 17.94 | C |
| ATOM | 762 | ND1 | HIS | A | 193 | −13.710 | 3.797 | −34.860 | 1.00 | 16.75 | N |
| ATOM | 763 | CE1 | HIS | A | 193 | −14.739 | 4.378 | −34.281 | 1.00 | 16.81 | C |
| ATOM | 764 | NE2 | HIS | A | 193 | −15.595 | 4.741 | −35.219 | 1.00 | 17.23 | N |
| ATOM | 765 | CD2 | HIS | A | 193 | −15.093 | 4.376 | −36.445 | 1.00 | 17.66 | C |
| ATOM | 766 | C | HIS | A | 193 | −11.401 | 3.551 | −39.093 | 1.00 | 17.45 | C |
| ATOM | 767 | O | HIS | A | 193 | −11.282 | 2.452 | −39.633 | 1.00 | 16.64 | O |
| ATOM | 768 | N | LEU | A | 194 | −10.412 | 4.433 | −38.977 | 1.00 | 17.22 | N |
| ATOM | 769 | CA | LEU | A | 194 | −9.040 | 4.135 | −39.382 | 1.00 | 17.30 | C |
| ATOM | 770 | CB | LEU | A | 194 | −8.179 | 5.406 | −39.345 | 1.00 | 17.60 | C |
| ATOM | 771 | CG | LEU | A | 194 | −6.675 | 5.284 | −39.642 | 1.00 | 18.15 | C |
| ATOM | 772 | CD1 | LEU | A | 194 | −6.483 | 4.839 | −41.054 | 1.00 | 18.50 | C |
| ATOM | 773 | CD2 | LEU | A | 194 | −5.955 | 6.571 | −39.374 | 1.00 | 18.92 | C |
| ATOM | 774 | C | LEU | A | 194 | −8.395 | 3.064 | −38.510 | 1.00 | 16.92 | C |
| ATOM | 775 | O | LEO | A | 194 | −7.796 | 2.137 | −39.043 | 1.00 | 15.92 | O |
| ATOM | 776 | N | ILE | A | 195 | −8.508 | 3.196 | −37.183 | 1.00 | 17.18 | N |
| ATOM | 777 | CA | ILE | A | 195 | −7.765 | 2.322 | −36.253 | 1.00 | 17.11 | C |
| ATOM | 778 | CB | ILE | A | 195 | −7.134 | 3.097 | −35.086 | 1.00 | 16.63 | C |
| ATOM | 779 | CG1 | ILE | A | 195 | −6.344 | 4.312 | −35.598 | 1.00 | 16.65 | C |
| ATOM | 780 | CD1 | ILE | A | 195 | −5.912 | 5.292 | −34.506 | 1.00 | 16.39 | C |
| ATOM | 781 | CG2 | ILE | A | 195 | −6.216 | 2.182 | −34.280 | 1.00 | 16.32 | C |
| ATOM | 782 | C | ILE | A | 195 | −6.678 | 1.225 | −35.715 | 1.00 | 17.97 | C |
| ATOM | 783 | O | ILE | A | 195 | −9.741 | 1.520 | −35.168 | 1.00 | 18.90 | O |
| ATOM | 784 | N | ARG | A | 196 | −8.253 | −0.026 | −35.879 | 1.00 | 17.74 | N |
| ATOM | 785 | CA | ARG | A | 196 | −8.885 | −1.170 | −35.247 | 1.00 | 17.88 | C |
| ATOM | 786 | CB | ARG | A | 196 | −9.289 | −2.214 | −36.288 | 1.00 | 18.06 | C |
| ATOM | 787 | CG | ARG | A | 196 | −10.263 | −1.722 | −37.321 | 1.00 | 18.58 | C |
| ATOM | 788 | CD | ARG | A | 196 | −10.555 | −2.826 | −38.319 | 1.00 | 19.07 | C |
| ATOM | 789 | NE | ARG | A | 196 | −11.495 | −2.389 | −39.361 | 1.00 | 19.35 | N |
| ATOM | 790 | CZ | ARG | A | 196 | −11.935 | −3.152 | −40.349 | 1.00 | 19.55 | C |
| ATOM | 791 | NH1 | ARG | A | 196 | −11.527 | −4.422 | −40.477 | 1.00 | 19.72 | N |
| ATOM | 792 | NH2 | ARG | A | 196 | −12.752 | −2.629 | −41.269 | 1.00 | 20.02 | N |
| ATOM | 793 | C | ARG | A | 196 | −7.916 | −1.831 | −34.292 | 1.00 | 17.62 | C |
| ATOM | 794 | O | ARG | A | 196 | −6.712 | −1.611 | −34.365 | 1.00 | 16.92 | O |
| ATOM | 795 | N | VAL | A | 197 | −8.472 | −2.645 | −33.402 | 1.00 | 17.79 | N |
| ATOM | 796 | CA | VAL | A | 197 | −7.698 | −3.566 | −32.595 | 1.00 | 18.33 | C |
| ATOM | 797 | CB | VAL | A | 197 | −7.980 | −3.401 | −31.093 | 1.00 | 17.86 | C |
| ATOM | 798 | CG1 | VAL | A | 197 | −7.288 | −4.505 | −30.286 | 1.00 | 17.46 | C |
| ATOM | 799 | CG2 | VAL | A | 197 | −7.538 | −2.020 | −30.631 | 1.00 | 17.47 | C |
| ATOM | 800 | C | VAL | A | 197 | −8.023 | −4.982 | −33.053 | 1.00 | 19.20 | C |
| ATOM | 801 | O | VAL | A | 197 | −9.177 | −5.334 | −33.259 | 1.00 | 18.30 | O |
| ATOM | 802 | N | GLU | A | 198 | −6.971 | −5.779 | −33.191 | 1.00 | 21.88 | N |
| ATOM | 803 | CA | GLU | A | 198 | −7.042 | −7.156 | −33.666 | 1.00 | 23.89 | C |
| ATOM | 804 | CB | GLU | A | 198 | −5.884 | −7.385 | −34.632 | 1.00 | 25.55 | C |
| ATOM | 805 | CG | GLU | A | 198 | −5.891 | −8.697 | −35.388 | 1.00 | 27.31 | C |
| ATOM | 806 | CD | GLU | A | 198 | −4.601 | −8.953 | −36.146 | 1.00 | 29.13 | C |
| ATOM | 807 | OE1 | GLU | A | 198 | −3.794 | −8.012 | −36.327 | 1.00 | 29.43 | O |
| ATOM | 808 | OE2 | GLU | A | 198 | −4.398 | −10.101 | −36.588 | 1.00 | 33.30 | O |
| ATOM | 809 | C | GLU | A | 198 | −6.912 | −8.096 | −32.466 | 1.00 | 24.34 | C |
| ATOM | 810 | O | GLU | A | 198 | −6.264 | −7.757 | −31.473 | 1.00 | 23.83 | O |
| ATOM | 811 | N | GLY | A | 199 | −7.539 | −9.268 | −32.558 | 1.00 | 24.92 | N |
| ATOM | 812 | CA | GLY | A | 199 | −7.403 | −10.322 | −31.542 | 1.00 | 24.91 | C |
| ATOM | 813 | C | GLY | A | 199 | −7.804 | −9.944 | −30.125 | 1.00 | 25.28 | C |
| ATOM | 814 | O | GLY | A | 199 | −7.172 | −10.384 | −29.173 | 1.00 | 24.74 | O |
| ATOM | 815 | N | ASN | A | 200 | −8.846 | −9.124 | −29.987 | 1.00 | 25.70 | N |
| ATOM | 816 | CA | ASN | A | 200 | −9.378 | −8.745 | −28.674 | 1.00 | 26.42 | C |
| ATOM | 817 | CB | ASN | A | 200 | −8.705 | −7.458 | −28.143 | 1.00 | 25.32 | C |
| ATOM | 818 | CG | ASN | A | 200 | −8.984 | −7.208 | −26.661 | 1.00 | 24.48 | C |
| ATOM | 819 | OD1 | ASN | A | 200 | −10.071 | −7.484 | −26.165 | 1.00 | 24.20 | O |
| ATOM | 820 | ND2 | ASN | A | 200 | −7.993 | −6.708 | −25.948 | 1.00 | 23.74 | N |
| ATOM | 821 | C | ASN | A | 200 | −10.898 | −8.580 | −28.765 | 1.00 | 27.46 | C |
| ATOM | 822 | O | ASN | A | 200 | −11.404 | −7.574 | −29.271 | 1.00 | 27.46 | O |
| ATOM | 823 | N | LEU | A | 201 | −11.611 | −9.570 | −28.241 | 1.00 | 28.95 | N |
| ATOM | 824 | CA | LEU | A | 201 | −13.079 | −9.626 | −28.333 | 1.00 | 30.09 | C |
| ATOM | 825 | CB | LEU | A | 201 | −13.586 | −11.045 | −28.053 | 1.00 | 31.85 | C |
| ATOM | 826 | CG | LEU | A | 201 | −14.951 | −11.414 | −28.668 | 1.00 | 33.70 | C |
| ATOM | 827 | CD1 | LEU | A | 201 | −14.831 | −11.598 | −30.176 | 1.00 | 33.61 | C |
| ATOM | 828 | CD2 | LEU | A | 201 | −15.532 | −12.663 | −28.011 | 1.00 | 34.81 | C |
| ATOM | 829 | C | LEU | A | 201 | −13.793 | −8.617 | −27.421 | 1.00 | 28.92 | C |
| ATOM | 830 | O | LEU | A | 201 | −14.978 | −8.357 | −27.611 | 1.00 | 28.45 | O |
| ATOM | 831 | N | ARG | A | 202 | −13.081 | −8.041 | −26.458 | 1.00 | 27.93 | N |
| ATOM | 832 | CA | ARG | A | 202 | −13.618 | −6.980 | −25.580 | 1.00 | 27.15 | C |
| ATOM | 833 | CB | ARG | A | 202 | −13.004 | −7.083 | −24.178 | 1.00 | 29.84 | C |
| ATOM | 834 | CG | ARG | A | 202 | −13.202 | −8.425 | −23.484 | 1.00 | 33.07 | C |
| ATOM | 835 | CD | ARG | A | 202 | −12.013 | −8.804 | −22.597 | 1.00 | 35.84 | C |
| ATOM | 836 | NE | ARG | A | 202 | −12.155 | −8.301 | −21.231 | 1.00 | 38.66 | N |
| ATOM | 837 | CZ | ARG | A | 202 | −11.171 | −8.185 | −20.342 | 1.00 | 39.60 | C |
| ATOM | 838 | NH1 | ARG | A | 202 | −9.904 | −8.491 | −20.652 | 1.00 | 40.42 | N |
| ATOM | 839 | NH2 | ARG | A | 202 | −11.454 | −7.726 | −19.121 | 1.00 | 40.52 | N |
| ATOM | 840 | C | ARG | A | 202 | −13.407 | −5.555 | −26.126 | 1.00 | 24.17 | C |
| ATOM | 841 | O | ARG | A | 202 | −13.627 | −4.584 | −25.418 | 1.00 | 23.00 | O |
| ATOM | 842 | N | VAL | A | 203 | −13.018 | −5.436 | −27.400 | 1.00 | 22.92 | N |
| ATOM | 843 | CA | VAL | A | 203 | −12.845 | −4.150 | −28.075 | 1.00 | 21.27 | C |
| ATOM | 844 | CB | VAL | A | 203 | −12.248 | −4.343 | −29.497 | 1.00 | 21.13 | C |
| ATOM | 845 | CG1 | VAL | A | 203 | −13.259 | −4.955 | −30.464 | 1.00 | 21.12 | C |
| ATOM | 846 | CG2 | VAL | A | 203 | −11.700 | −3.043 | −30.049 | 1.00 | 21.27 | C |
| ATOM | 847 | C | VAL | A | 203 | −14.156 | −3.345 | −28.148 | 1.00 | 20.47 | C |
| ATOM | 848 | O | VAL | A | 203 | −15.238 | −3.910 | −28.324 | 1.00 | 19.25 | O |
| ATOM | 849 | N | GLU | A | 204 | −14.033 | −2.026 | −28.006 | 1.00 | 19.21 | N |
| ATOM | 850 | CA | GLU | A | 204 | −15.169 | −1.111 | −28.045 | 1.00 | 18.91 | C |
| ATOM | 851 | CB | GLU | A | 204 | −15.525 | −0.667 | −26.623 | 1.00 | 19.25 | C |
| ATOM | 852 | CG | GLU | A | 204 | −16.438 | 0.551 | −26.491 | 1.00 | 19.28 | C |
| ATOM | 853 | CD | GLU | A | 204 | −16.601 | 1.008 | −25.048 | 1.00 | 20.01 | C |
| ATOM | 854 | OE1 | GLU | A | 204 | −16.688 | 0.132 | −24.159 | 1.00 | 19.46 | O |
| ATOM | 855 | OE2 | GLU | A | 204 | −16.631 | 2.248 | −24.804 | 1.00 | 19.92 | O |
| ATOM | 856 | C | GLU | A | 204 | −14.811 | 0.087 | −28.908 | 1.00 | 18.07 | C |
| ATOM | 857 | O | GLU | A | 204 | −13.726 | 0.643 | −28.770 | 1.00 | 17.52 | O |
| ATOM | 858 | N | TYR | A | 205 | −15.741 | 0.485 | −29.777 | 1.00 | 17.20 | N |
| ATOM | 859 | CA | TYR | A | 205 | −15.562 | 1.633 | −30.661 | 1.00 | 16.86 | C |
| ATOM | 860 | CB | TYR | A | 205 | −15.769 | 1.210 | −32.108 | 1.00 | 15.56 | C |
| ATOM | 861 | CG | TYR | A | 205 | −14.698 | 0.261 | −32.589 | 1.00 | 14.69 | C |
| ATOM | 862 | CD1 | TYR | A | 205 | −13.502 | 0.737 | −33.127 | 1.00 | 14.15 | C |
| ATOM | 863 | CE1 | TYR | A | 205 | −12.538 | −0.142 | −33.595 | 1.00 | 13.84 | C |
| ATOM | 864 | CZ | TYR | A | 205 | −12.718 | −1.503 | −33.450 | 1.00 | 13.55 | C |
| ATOM | 865 | OH | TYR | A | 205 | −11.735 | −2.353 | −33.886 | 1.00 | 12.87 | O |
| ATOM | 866 | CE2 | TYR | A | 205 | −13.878 | −1.997 | −32.883 | 1.00 | 13.43 | C |
| ATOM | 867 | CD2 | TYR | A | 205 | −14.874 | −1.122 | −32.491 | 1.00 | 13.87 | C |
| ATOM | 868 | C | TYR | A | 205 | −16.512 | 2.774 | −30.307 | 1.00 | 17.68 | C |
| ATOM | 869 | O | TYR | A | 205 | −17.646 | 2.535 | −29.875 | 1.00 | 18.09 | O |
| ATOM | 870 | N | LEU | A | 206 | −16.032 | 4.001 | −30.506 | 1.00 | 18.13 | N |
| ATOM | 871 | CA | LEU | A | 206 | −16.776 | 5.214 | −30.182 | 1.00 | 19.21 | C |
| ATOM | 872 | CB | LEU | A | 206 | −16.180 | 5.882 | −28.932 | 1.00 | 19.84 | C |
| ATOM | 873 | CG | LEU | A | 206 | −16.822 | 7.165 | −28.373 | 1.00 | 20.33 | C |
| ATOM | 874 | CD1 | LEU | A | 206 | −18.203 | 6.886 | −27.794 | 1.00 | 20.39 | C |
| ATOM | 875 | CD2 | LEU | A | 206 | −15.929 | 7.811 | −27.321 | 1.00 | 20.70 | C |
| ATOM | 876 | C | LEU | A | 206 | −16.740 | 6.199 | −31.344 | 1.00 | 20.05 | C |
| ATOM | 877 | O | LEU | A | 206 | −15.674 | 6.495 | −31.895 | 1.00 | 19.01 | O |
| ATOM | 878 | N | ASP | A | 207 | −17.925 | 6.659 | −31.733 | 1.00 | 20.97 | N |
| ATOM | 879 | CA | ASP | A | 207 | −18.078 | 7.905 | −32.458 | 1.00 | 22.38 | C |
| ATOM | 880 | CB | ASP | A | 207 | −19.191 | 7.800 | −33.503 | 1.00 | 22.85 | C |
| ATOM | 881 | CG | ASP | A | 207 | −18.849 | 6.863 | −34.650 | 1.00 | 23.55 | C |
| ATOM | 882 | OD1 | ASP | A | 207 | −17.688 | 6.417 | −34.777 | 1.00 | 25.31 | O |
| ATOM | 883 | OD2 | ASP | A | 207 | −19.761 | 6.586 | −35.449 | 1.00 | 23.53 | O |
| ATOM | 884 | C | ASP | A | 207 | −16.485 | 8.902 | −31.394 | 1.00 | 22.26 | C |
| ATOM | 885 | O | ASP | A | 207 | −19.554 | 8.757 | −30.791 | 1.00 | 21.34 | O |
| ATOM | 886 | N | ASP | A | 208 | −17.633 | 9.888 | −31.129 | 1.00 | 24.18 | N |
| ATOM | 887 | CA | ASP | A | 208 | −17.908 | 10.847 | −30.060 | 1.00 | 26.01. | C |
| ATOM | 888 | CB | ASP | A | 208 | −16.684 | 11.706 | −29.772 | 1.00 | 25.85 | C |
| ATOM | 889 | CG | ASP | A | 208 | −16.826 | 12.529 | −28.494 | 1.00 | 26.09 | C |
| ATOM | 890 | OD1 | ASP | A | 208 | −17.633 | 13.482 | −28.478 | 1.00 | 26.33 | O |
| ATOM | 891 | OD2 | ASP | A | 208 | −16.137 | 12.232 | −27.501 | 1.00 | 25.77 | O |
| ATOM | 892 | C | ASP | A | 208 | −19.104 | 11.709 | −30.466 | 1.00 | 27.83 | C |
| ATOM | 893 | O | ASP | A | 208 | −19.130 | 12.240 | −31.568 | 1.00 | 26.45 | O |
| ATOM | 894 | N | ARG | A | 209 | −20.085 | 11.816 | −29.577 | 1.00 | 31.42 | N |
| ATOM | 895 | CA | ARG | A | 209 | −21.337 | 12.530 | −29.850 | 1.00 | 36.19 | C |
| ATOM | 896 | CB | ARG | A | 209 | −22.391 | 12.192 | −28.775 | 1.00 | 40.24 | C |
| ATOM | 897 | CG | ARG | A | 209 | −22.116 | 12.776 | −27.392 | 1.00 | 45.03 | C |
| ATOM | 896 | CD | ARG | A | 209 | −22.329 | 11.827 | −26.214 | 1.00 | 49.05 | C |
| ATOM | 899 | NE | ARG | A | 209 | −21.683 | 12.391 | −25.022 | 1.00 | 53.23 | N |
| ATOM | 900 | CZ | ARG | A | 209 | −21.330 | 11.715 | −23.922 | 1.00 | 56.73 | C |
| ATOM | 901 | NH1 | ARG | A | 209 | −21.553 | 10.400 | −23.799 | 1.00 | 56.85 | N |
| ATOM | 902 | NH2 | ARG | A | 209 | −20.742 | 12.370 | −22.916 | 1.00 | 57.69 | N |
| ATOM | 903 | C | ARG | A | 209 | −21.190 | 14.058 | −29.997 | 1.00 | 34.97 | C |
| ATOM | 904 | O | ARG | A | 209 | −22.028 | 14.679 | −30.647 | 1.00 | 36.66 | O |
| ATOM | 905 | N | ASN | A | 210 | −20.142 | 14.648 | −29.418 | 1.00 | 33.98 | N |
| ATOM | 906 | CA | ASN | A | 210 | −19.893 | 16.096 | −29.529 | 1.00 | 34.16 | C |
| ATOM | 907 | CB | ASN | A | 210 | −19.441 | 16.671 | −28.180 | 1.00 | 35.00 | C |
| ATOM | 908 | CG | ASN | A | 210 | −20.423 | 16.391 | −27.059 | 1.00 | 35.97 | C |
| ATOM | 909 | OD1 | ASN | A | 210 | −20.023 | 16.161 | −25.917 | 1.00 | 36.80 | O |
| ATOM | 910 | ND2 | ASN | A | 210 | −21.714 | 16.403 | −27.379 | 1.00 | 34.70 | N |
| ATOM | 911 | C | ASN | A | 210 | −18.869 | 16.455 | −30.611 | 1.00 | 32.61 | C |
| ATOM | 912 | O | ASN | A | 210 | −19.098 | 17.382 | −31.384 | 1.00 | 31.29 | O |
| ATOM | 913 | N | THR | A | 211 | −17.742 | 15.739 | −30.646 | 1.00 | 32.25 | N |
| ATOM | 914 | CA | THR | A | 211 | −16.639 | 16.032 | −31.581 | 1.00 | 31.29 | C |
| ATOM | 915 | CB | THR | A | 211 | −15.268 | 15.768 | −30.926 | 1.00 | 31.79 | C |
| ATOM | 916 | OG1 | THR | A | 211 | −15.136 | 14.368 | −30.622 | 1.00 | 31.71 | O |
| ATOM | 917 | CG2 | THR | A | 211 | −15.098 | 16.592 | −29.647 | 1.00 | 31.77 | C |
| ATOM | 918 | C | THR | A | 211 | −16.676 | 15.228 | −32.883 | 1.00 | 29.70 | C |
| ATOM | 919 | O | THR | A | 211 | −15.981 | 15.575 | −33.836 | 1.00 | 29.62 | O |
| ATOM | 920 | N | PHE | A | 212 | −17.451 | 14.140 | −32.901 | 1.00 | 28.66 | N |
| ATOM | 921 | CA | PHE | A | 212 | −17.539 | 13.199 | −34.042 | 1.00 | 27.06 | C |
| ATOM | 922 | CB | PHE | A | 212 | −18.042 | 13.897 | −35.321 | 1.00 | 28.21 | C |
| ATOM | 923 | CG | PHE | A | 212 | −19.231 | 14.791 | −35.097 | 1.00 | 29.49 | C |
| ATOM | 924 | CD1 | PHE | A | 212 | −20.452 | 14.254 | −34.708 | 1.00 | 30.10 | C |
| ATOM | 925 | CE1 | PHE | A | 212 | −21.548 | 15.069 | −34.459 | 1.00 | 30.97 | C |
| ATOM | 926 | CZ | PHE | A | 212 | −21.444 | 16.442 | −34.657 | 1.00 | 31.48 | C |
| ATOM | 927 | CE2 | PHE | A | 212 | −20.229 | 16.996 | −35.055 | 1.00 | 31.03 | C |
| ATOM | 928 | CD2 | PHE | A | 212 | −19.132 | 16.172 | −35.270 | 1.00 | 30.31 | C |
| ATOM | 929 | C | PHE | A | 212 | −16.252 | 12.420 | −34.324 | 1.00 | 24.62 | C |
| ATOM | 930 | O | PHE | A | 212 | −16.178 | 11.730 | −35.339 | 1.00 | 22.97 | O |
| ATOM | 931 | N | ARG | A | 213 | −15.271 | 12.477 | −33.420 | 1.00 | 23.68 | N |
| ATOM | 932 | CA | ARG | A | 213 | −13.992 | 11.806 | −33.629 | 1.00 | 22.16 | C |
| ATOM | 933 | CB | ARG | A | 213 | −12.877 | 12.492 | −32.856 | 1.00 | 22.21 | C |
| ATOM | 934 | CG | ARG | A | 213 | −12.518 | 13.856 | −33.408 | 1.00 | 22.36 | C |
| ATOM | 935 | CD | ARG | A | 213 | −11.319 | 14.467 | −32.641 | 1.00 | 22.22 | C |
| ATOM | 936 | NE | ARG | A | 213 | −10.942 | 15.799 | −33.130 | 1.00 | 22.37 | N |
| ATOM | 937 | CZ | ARG | A | 213 | −10.188 | 16.059 | −34.213 | 1.00 | 21.59 | C |
| ATOM | 938 | NH1 | ARG | A | 213 | −9.659 | 15.086 | −34.952 | 1.00 | 21.35 | N |
| ATOM | 939 | NH2 | ARG | A | 213 | −9.928 | 17.321 | −34.539 | 1.00 | 20.82 | N |
| ATOM | 940 | C | ARG | A | 213 | −14.102 | 10.319 | −33.261 | 1.00 | 21.62 | C |
| ATOM | 941 | O | ARG | A | 213 | −14.932 | 9.928 | −32.411 | 1.00 | 19.77 | O |
| ATOM | 942 | N | HIS | A | 214 | −13.284 | 9.513 | −33.929 | 1.00 | 20.83 | N |
| ATOM | 943 | CA | HIS | A | 214 | −13.338 | 8.061 | −33.809 | 1.00 | 21.12 | C |
| ATOM | 944 | CB | HIS | A | 214 | −13.147 | 7.396 | −35.171 | 1.00 | 20.81 | C |
| ATOM | 945 | CG | HIS | A | 214 | −14.189 | 7.774 | −36.176 | 1.00 | 21.18 | C |
| ATOM | 946 | ND1 | HIS | A | 214 | −13.986 | 7.682 | −37.538 | 1.00 | 20.90 | N |
| ATOM | 947 | CE1 | HIS | A. | 214 | −15.076 | 8.079 | −38.170 | 1.00 | 20.29 | C |
| ATOM | 948 | NE2 | HIS | A | 214 | −15.961 | 8.459 | −37.269 | 1.00 | 20.35 | N |
| ATOM | 949 | CD2 | HIS | A | 214 | −15.443 | 8.258 | −36.016 | 1.00 | 20.81 | C |
| ATOM | 950 | C | HIS | A | 214 | −12.260 | 7.581 | −32.856 | 1.00 | 21.06 | C |
| ATOM | 951 | O | HIS | A | 214 | −11.143 | 8.112 | −32.848 | 1.00 | 22.00 | O |
| ATOM | 952 | N | SER | A | 215 | −12.592 | 6.586 | −32.044 | 1.00 | 20.57 | N |
| ATOM | 953 | CA | SER | A | 215 | −11.585 | 5.949 | −31.206 | 1.00 | 20.71 | C |
| ATOM | 954 | CB | SER | A | 215 | −11.418 | 6.712 | −29.885 | 1.00 | 20.73 | C |
| ATOM | 955 | OG | SER | A | 215 | −12.617 | 6.723 | −29.130 | 1.00 | 20.44 | O |
| ATOM | 956 | C | SER | A | 215 | −11.924 | 4.491 | −30.944 | 1.00 | 20.24 | C |
| ATOM | 957 | O | SER | A | 215 | −13.063 | 4.057 | −31.149 | 1.00 | 21.31 | O |
| ATOM | 958 | N | VAL | A | 216 | −10.922 | 3.749 | −30.490 | 1.00 | 19.27 | N |
| ATOM | 959 | CA | VAL | A | 216 | −11.071 | 2.340 | −30.147 | 1.00 | 19.91 | C |
| ATOM | 960 | CB | VAL | A | 216 | −10.457 | 1.419 | −31.227 | 1.00 | 19.62 | C |
| ATOM | 961 | CG1 | VAL | A | 216 | −8.983 | 1.707 | −31.462 | 1.00 | 19.77 | C |
| ATOM | 962 | CG2 | VAL | A | 216 | −10.654 | −0.040 | −30.880 | 1.00 | 19.48 | C |
| ATOM | 963 | C | VAL | A | 216 | −10.455 | 2.114 | −28.769 | 1.00 | 20.87 | C |
| ATOM | 964 | O | VAL | A | 216 | −9.341 | 2.562 | −28.499 | 1.00 | 20.45 | O |
| ATOM | 965 | N | VAL | A | 217 | −11.189 | 1.421 | −27.904 | 1.00 | 21.79 | N |
| ATOM | 966 | CA | VAL | A | 217 | −10.765 | 1.216 | −26.520 | 1.00 | 23.38 | C |
| ATOM | 967 | CB | VAL | A | 217 | −11.572 | 2.125 | −25.548 | 1.00 | 23.93 | C |
| ATOM | 968 | CG1 | VAL | A | 217 | −13.065 | 1.842 | −25.601 | 1.00 | 24.52 | C |
| ATOM | 969 | CG2 | VAL | A | 217 | −11.073 | 1.986 | −24.129 | 1.00 | 24.25 | C |
| ATOM | 970 | C | VAL | A | 217 | −10.809 | −0.274 | −26.154 | 1.00 | 23.54 | C |
| ATOM | 971 | O | VAL | A | 217 | −11.677 | −0.995 | −26.609 | 1.00 | 23.78 | O |
| ATOM | 972 | N | VAL | A | 218 | −9.829 | −0.721 | −25.374 | 1.00 | 24.00 | N |
| ATOM | 973 | CA | VAL | A | 218 | −9.784 | −2.086 | −24.827 | 1.00 | 24.86 | C |
| ATOM | 974 | CB | VAL | A | 218 | −8.740 | −3.001 | −25.522 | 1.00 | 25.44 | C |
| ATOM | 975 | CG1 | VAL | A | 218 | −9.152 | −3.312 | −26.948 | 1.00 | 25.49 | C |
| ATOM | 976 | CG2 | VAL | A | 218 | −7.340 | −2.394 | −25.499 | 1.00 | 25.79 | C |
| ATOM | 977 | C | VAL | A | 218 | −9.471 | −2.018 | −23.335 | 1.00 | 25.11 | C |
| ATOM | 978 | O | VAL | A | 218 | −8.941 | −1.002 | −22.868 | 1.00 | 23.40 | O |
| ATOM | 979 | N | PRO | A | 219 | −9.807 | −3.086 | −22.580 | 1.00 | 25.83 | N |
| ATOM | 980 | CA | PRO | A | 319 | −9.386 | −3.103 | −21.175 | 1.00 | 26.19 | C |
| ATOM | 981 | CB | PRO | A | 219 | −10.051 | −4.359 | −20.616 | 1.00 | 26.06 | C |
| ATOM | 982 | CG | PRO | A | 219 | −11.179 | −4.672 | −21.545 | 1.00 | 26.08 | C |
| ATOM | 983 | CD | PRO | A | 219 | −10.723 | −4.201 | −22.895 | 1.00 | 25.48 | C |
| ATOM | 984 | C | PRO | A | 219 | −7.869 | −3.212 | −21.057 | 1.00 | 26.90 | C |
| ATOM | 985 | O | PRO | A | 219 | −7.238 | −3.985 | −21.804 | 1.00 | 27.28 | O |
| ATOM | 986 | N | TYR | A | 220 | −7.286 | −2.425 | −20.156 | 1.00 | 28.01 | N |
| ATOM | 987 | CA | TYR | A | 220 | −5.899 | −2.614 | −19.783 | 1.00 | 28.34 | C |
| ATOM | 988 | CB | TYR | A | 220 | −5.387 | −1.477 | −18.902 | 1.00 | 27.84 | C |
| ATOM | 989 | CG | TYR | A | 220 | −3.968 | −1.733 | −18.413 | 1.00 | 27.67 | C |
| ATOM | 990 | CD1 | TYR | A | 220 | −2.864 | −1.475 | −19.239 | 1.00 | 27.18 | C |
| ATOM | 991 | CE1 | TYR | A | 220 | −1.573 | −1.737 | −18.822 | 1.00 | 27.95 | C |
| ATOM | 992 | CZ | TYR | A | 220 | −1.362 | −2.268 | −17.555 | 1.00 | 27.95 | C |
| ATOM | 993 | OH | TYR | A | 220 | −0.100 | −2.523 | −17.098 | 1.00 | 27.92 | O |
| ATOM | 994 | CE2 | TYR | A | 220 | −2.441 | −2.554 | −16.724 | 1.00 | 27.41 | C |
| ATOM | 995 | CD2 | TYR | A | 220 | −3.725 | −2.291 | −17.148 | 1.00 | 27.40 | C |
| ATOM | 996 | C | TYR | A | 220 | −5.765 | −3.940 | −19.034 | 1.00 | 29.34 | C |
| ATOM | 997 | O | TYR | A | 220 | −6.400 | −4.119 | −18.002 | 1.00 | 29.85 | O |
| ATOM | 998 | N | GLU | A | 221 | −4.961 | −4.850 | −19.574 | 1.00 | 31.95 | N |
| ATOM | 999 | CA | GLU | A | 221 | −4.549 | −6.048 | −18.877 | 1.00 | 33.95 | C |
| ATOM | 1000 | CB | GLU | A | 221 | −4.781 | −7.297 | −19.742 | 1.00 | 35.95 | C |
| ATOM | 1001 | CG | GLU | A | 221 | −6.252 | −7.712 | −19.860 | 1.00 | 37.82 | C |
| ATOM | 1002 | CD | GLU | A | 221 | −6.873 | −8.158 | −18.528 | 1.00 | 40.45 | C |
| ATOM | 1003 | OE1 | GLU | A | 221 | −6.146 | −8.622 | −17.638 | 1.00 | 40.02 | O |
| ATOM | 1004 | OE2 | GLU | A | 221 | −8.114 | −8.082 | −18.383 | 1.00 | 42.96 | O |
| ATOM | 1005 | C | GLU | A | 221 | −3.070 | −5.887 | −18.545 | 1.00 | 33.80 | C |
| ATOM | 1006 | O | GLU | A | 221 | −2.306 | −5.379 | −19.367 | 1.00 | 32.00 | O |
| ATOM | 1007 | N | PRO | A | 222 | −2.656 | −6.282 | −17.317 | 1.00 | 33.86 | N |
| ATOM | 1008 | CA | PRO | A | 222 | −1.237 | −6.195 | −17.004 | 1.00 | 33.86 | C |
| ATOM | 1009 | CB | PRO | A | 222 | −1.182 | −6.502 | −15.497 | 1.00 | 33.50 | C |
| ATOM | 1010 | CG | PRO | A | 222 | −2.456 | −7.183 | −15.171 | 1.00 | 33.63 | C |
| ATOM | 1011 | CD | PRO | A | 222 | −3.451 | −6.628 | −16.127 | 1.00 | 32.95 | C |
| ATOM | 1012 | C | PRO | A | 222 | −0.400 | −7.199 | −17.816 | 1.00 | 34.68 | C |
| ATOM | 1013 | O | PRO | A | 222 | −0.958 | −8.147 | −18.380 | 1.00 | 34.49 | O |
| ATOM | 1014 | N | PRO | A | 223 | 0.937 | −6.990 | −17.876 | 1.00 | 36.21 | N |
| ATOM | 1015 | CA | PRO | A | 223 | 1.823 | −7.875 | −18.633 | 1.00 | 37.08 | C |
| ATOM | 1016 | CB | PRO | A | 223 | 3.227 | −7.403 | −18.206 | 1.00 | 35.81 | C |
| ATOM | 1017 | CG | PRO | A | 223 | 3.065 | −6.024 | −17.753 | 1.00 | 36.03 | C |
| ATOM | 1018 | CD | PRO | A | 223 | 1.673 | −5.872 | −17.246 | 1.00 | 35.62 | C |
| ATOM | 1019 | C | PRO | A | 223 | 1.635 | −9.371 | −18.262 | 1.00 | 39.24 | C |
| ATOM | 1020 | O | PRO | A | 223 | 1.325 | −9.698 | −17.102 | 1.00 | 39.32 | O |
| ATOM | 1021 | N | GLU | A | 224 | 1.838 | −10.249 | −19.225 | 1.00 | 41.89 | N |
| ATOM | 1022 | CA | GLU | A | 224 | 1.886 | −11.695 | −18.975 | 1.00 | 44.32 | C |
| ATOM | 1023 | CB | GLU | A | 224 | 2.021 | −12.545 | −20.261 | 1.00 | 44.60 | C |
| ATOM | 1024 | CG | GLU | A | 224 | 1.156 | −12.256 | −21.470 | 1.00 | 46.73 | C |
| ATOM | 1025 | CD | GLU | A | 224 | −0.334 | −12.363 | −21.239 | 1.00 | 48.27 | C |
| ATOM | 1026 | OE1 | GLU | A | 224 | −1.053 | −12.107 | −22.229 | 1.00 | 49.06 | O |
| ATOM | 1027 | OE2 | GLU | A | 224 | −0.816 | −12.712 | −20.135 | 1.00 | 49.60 | O |
| ATOM | 1028 | C | GLU | A | 224 | 3.083 | −12.036 | −18.083 | 1.00 | 45.08 | C |
| ATOM | 1029 | O | GLU | A | 224 | 4.007 | −11.216 | −17.917 | 1.00 | 44.01 | O |
| ATOM | 1030 | N | VAL | A | 225 | 3.057 | −13.223 | −17.482 | 1.00 | 46.39 | N |
| ATOM | 1031 | CA | VAL | A | 225 | 4.091 | −13.580 | −16.502 | 1.00 | 46.48 | C |
| ATOM | 1032 | CB | VAL | A | 225 | 3.719 | −14.821 | −15.660 | 1.00 | 47.76 | C |
| ATOM | 1033 | CG1 | VAL | A | 225 | 4.808 | −15.092 | −14.652 | 1.00 | 48.35 | C |
| ATOM | 1034 | CG2 | VAL | A | 225 | 2.389 | −14.627 | −14.936 | 1.00 | 48.38 | C |
| ATOM | 1035 | C | VAL | A | 225 | 5.408 | −13.754 | −17.276 | 1.00 | 45.98 | C |
| ATOM | 1036 | O | VAL | A | 225 | 5.435 | −14.453 | −18.288 | 1.00 | 46.07 | O |
| ATOM | 1037 | N | GLY | A | 226 | 6.468 | −13.075 | −16.829 | 1.00 | 44.65 | N |
| ATOM | 1038 | CA | GLY | A | 226 | 7.738 | −13.027 | −17.556 | 1.00 | 43.62 | C |
| ATOM | 1039 | C | GLY | A | 226 | 7.980 | −11.780 | −18.396 | 1.00 | 42.12 | C |
| ATOM | 1040 | O | GLY | A | 226 | 9.125 | −11.457 | −18.684 | 1.00 | 42.32 | O |
| ATOM | 1041 | N | SER | A | 227 | 6.910 | −11.069 | −18.769 | 1.00 | 39.83 | N |
| ATOM | 1042 | CA | SER | A | 227 | 7.012 | −9.841 | −19.574 | 1.00 | 39.12 | C |
| ATOM | 1043 | CB | SER | A | 227 | 5.881 | −9.787 | −20.600 | 1.00 | 39.98 | C |
| ATOM | 1044 | OG | SER | A | 227 | 5.956 | −8.698 | −21.483 | 1.00 | 41.19 | O |
| ATOM | 1045 | C | SER | A | 227 | 6.956 | −8.587 | −18.707 | 1.00 | 36.72 | C |
| ATOM | 1046 | O | SER | A | 227 | 6.458 | −8.615 | −17.583 | 1.00 | 36.75 | O |
| ATOM | 1047 | N | ASP | A | 228 | 7.517 | −7.498 | −19.232 | 1.00 | 35.16 | N |
| ATOM | 1048 | CA | ASP | A | 228 | 7.491 | −6.189 | −18.576 | 1.00 | 34.42 | C |
| ATOM | 1049 | CB | ASP | A | 228 | 8.898 | −5.587 | −18.509 | 1.00 | 36.51 | C |
| ATOM | 1050 | CG | ASP | A | 228 | 9.843 | −6.352 | −17.584 | 1.00 | 38.07 | C |
| ATOM | 1051 | OD1 | ASP | A | 228 | 9.390 | −7.181 | −16.753 | 1.00 | 38.82 | O |
| ATOM | 1052 | OD2 | ASP | A | 228 | 11.063 | −6.128 | −17.721 | 1.00 | 39.67 | O |
| ATOM | 1053 | C | ASP | A | 228 | 6.531 | −5.204 | −19.267 | 1.00 | 33.10 | C |
| ATOM | 1054 | O | ASP | A | 228 | 6.440 | −4.046 | −18.812 | 1.00 | 32.96 | O |
| ATOM | 1055 | N | CYS | A | 229 | 5.805 | −5.660 | −20.305 | 1.00 | 30.24 | N |
| ATOM | 1056 | CA | CYS | A | 229 | 4.870 | −4.784 | −21.002 | 1.00 | 28.92 | C |
| ATOM | 1057 | CB | CYS | A | 229 | 5.570 | −4.043 | −22.153 | 1.00 | 29.34 | C |
| ATOM | 1058 | SG | CYS | A | 229 | 6.171 | −5.096 | −23.468 | 1.00 | 29.26 | S |
| ATOM | 1059 | C | CYS | A | 229 | 3.649 | −5.511 | −21.532 | 1.00 | 26.95 | C |
| ATOM | 1060 | O | CYS | A | 229 | 3.653 | −6.722 | −21.652 | 1.00 | 25.84 | O |
| ATOM | 1061 | N | THR | A | 230 | 2.610 | −4.729 | −21.821 | 1.00 | 25.20 | N |
| ATOM | 1062 | CA | THR | A | 230 | 1.380 | −5.227 | −22.446 | 1.00 | 23.91 | C |
| ATOM | 1063 | CB | THR | A | 230 | 0.132 | −4.629 | −21.781 | 1.00 | 23.35 | C |
| ATOM | 1064 | OG1 | THR | A | 230 | 0.181 | −4.827 | −20.374 | 1.00 | 23.02 | O |
| ATOM | 1065 | CG2 | THR | A | 230 | −1.133 | −5.246 | −22.349 | 1.00 | 22.86 | C |
| ATOM | 1066 | C | THR | A | 230 | 1.377 | −4.802 | −23.911 | 1.00 | 22.85 | C |
| ATOM | 1067 | O | THR | A | 230 | 1.598 | −3.627 | −24.216 | 1.00 | 23.29 | O |
| ATOM | 1068 | N | THR | A | 231 | 1.076 | −5.752 | −24.799 | 1.00 | 22.22 | N |
| ATOM | 1069 | CA | THR | A | 231 | 1.091 | −5.525 | −26.244 | 1.00 | 22.26 | C |
| ATOM | 1070 | CB | THR | A | 231 | 1.955 | −6.593 | −26.945 | 1.00 | 21.90 | C |
| ATOM | 1071 | OG1 | THR | A | 231 | 3.287 | −6.537 | −26.420 | 1.00 | 21.44 | O |
| ATOM | 1072 | CG2 | THR | A | 231 | 2.033 | −6.354 | −28.433 | 1.00 | 22.14 | C |
| ATOM | 1073 | C | THR | A | 231 | −0.329 | −5.553 | −26.820 | 1.00 | 22.06 | C |
| ATOM | 1074 | O | THR | A | 231 | −1.055 | −6.536 | −26.657 | 1.00 | 22.57 | O |
| ATOM | 1075 | N | ILE | A | 232 | −0.704 | −4.466 | −27.496 | 1.00 | 21.44 | N |
| ATOM | 1076 | CA | ILE | A | 232 | −1.962 | −4.385 | −28.242 | 1.00 | 20.29 | C |
| ATOM | 1077 | CB | ILE | A | 232 | −2.726 | −3.090 | −27.916 | 1.00 | 19.89 | C |
| ATOM | 1078 | CG1 | ILE | A | 232 | −3.168 | −3.103 | −26.442 | 1.00 | 20.42 | C |
| ATOM | 1079 | CD1 | ILE | A | 232 | −3.680 | −1.761 | −25.908 | 1.00 | 21.13 | C |
| ATOM | 1080 | CG2 | ILE | A | 232 | −3.937 | −2.929 | −28.810 | 1.00 | 19.21 | C |
| ATOM | 1081 | C | ILE | A | 232 | −1.610 | −4.448 | −29.735 | 1.00 | 20.49 | C |
| ATOM | 1082 | O | ILE | A | 232 | −0.616 | −3.853 | −30.169 | 1.00 | 20.04 | O |
| ATOM | 1083 | N | HIS | A | 233 | −2.433 | −5.158 | −30.509 | 1.00 | 20.17 | N |
| ATOM | 1084 | CA | HIS | A | 233 | −2.251 | −5.259 | −31.957 | 1.00 | 20.49 | C |
| ATOM | 1085 | CB | HIS | A | 233 | −2.426 | −6.698 | −32.449 | 1.00 | 22.09 | C |
| ATOM | 1086 | CG | HIS | A | 233 | −1.305 | −7.612 | −32.069 | 1.00 | 23.87 | C |
| ATOM | 1087 | ND1 | HIS | A | 233 | −1.421 | −8.987 | −32.112 | 1.00 | 25.32 | N |
| ATOM | 1088 | CE1 | HIS | A | 233 | −0.280 | −9.536 | −31.731 | 1.00 | 25.65 | C |
| ATOM | 1089 | NE2 | HIS | A | 233 | 0.571 | −8.567 | −31.445 | 1.00 | 26.18 | N |
| ATOM | 1090 | CD2 | HIS | A | 233 | −0.045 | −7.355 | −31.650 | 1.00 | 24.88 | C |
| ATOM | 1091 | C | HIS | A | 233 | −3.242 | −4.353 | −32.676 | 1.00 | 19.27 | C |
| ATOM | 1092 | O | HIS | A | 233 | −4.424 | −4.685 | −32.778 | 1.00 | 19.13 | O |
| ATOM | 1093 | N | TYR | A | 234 | −2.746 | −3.216 | −33.172 | 1.00 | 17.60 | N |
| ATOM | 1094 | CA | TYR | A | 234 | −3.550 | −2.294 | −33.958 | 1.00 | 17.30 | C |
| ATOM | 1095 | CB | TYR | A | 234 | −3.117 | −0.845 | −33.710 | 1.00 | 16.76 | C |
| ATOM | 1096 | CG | TYR | A | 234 | −3.418 | −0.342 | −32.311 | 1.00 | 16.80 | C |
| ATOM | 1097 | CD1 | TYR | A | 234 | −4.732 | −0.070 | −31.907 | 1.00 | 16.69 | C |
| ATOM | 1098 | CE1 | TYR | A | 234 | −5.014 | 0.397 | −30.629 | 1.00 | 16.60 | C |
| ATOM | 1099 | CZ | TYR | A | 234 | −3.982 | 0.595 | −29.733 | 1.00 | 16.72 | C |
| ATOM | 1100 | OH | TYR | A | 234 | −4.252 | 1.051 | −28.467 | 1.00 | 17.27 | O |
| ATOM | 1101 | CE2 | TYR | A | 234 | −2.664 | 0.344 | −30.109 | 1.00 | 16.70 | C |
| ATOM | 1102 | CD2 | TYR | A | 234 | −2.396 | −0.121 | −31.392 | 1.00 | 16.78 | C |
| ATOM | 1103 | C | TYR | A | 234 | −3.457 | −2.626 | −35.452 | 1.00 | 17.12 | C |
| ATOM | 1104 | O | TYR | A | 234 | −2.472 | −3.213 | −35.911 | 1.00 | 17.37 | O |
| ATOM | 1105 | N | ASN | A | 235 | −4.504 | −2.273 | −36.192 | 1.00 | 17.25 | N |
| ATOM | 1106 | CA | ASN | A | 235 | −4.487 | −2.305 | −37.657 | 1.00 | 17.45 | C |
| ATOM | 1107 | CB | ASN | A | 235 | −5.456 | −3.341 | −38.229 | 1.00 | 18.07 | C |
| ATOM | 1108 | CG | ASN | A | 235 | −5.273 | −4.736 | −37.654 | 1.00 | 18.97 | C |
| ATOM | 1109 | OD1 | ASN | A | 235 | −6.248 | −5.461 | −37.476 | 1.00 | 19.73 | O |
| ATOM | 1110 | ND2 | ASN | A | 235 | −4.034 | −5.123 | −37.374 | 1.00 | 19.06 | N |
| ATOM | 1111 | C | ASN | A | 235 | −4.904 | −0.923 | −38.154 | 1.00 | 16.80 | C |
| ATOM | 1112 | O | ASN | A | 235 | −5.855 | −0.349 | −37.639 | 1.00 | 15.86 | O |
| ATOM | 1113 | N | TYR | A | 236 | −4.198 | −0.417 | −39.159 | 1.00 | 16.93 | N |
| ATOM | 1114 | CA | TYR | A | 236 | −4.558 | 0.830 | −39.826 | 1.00 | 17.98 | C |
| ATOM | 1115 | CB | TYR | A | 236 | −3.338 | 1.733 | −39.940 | 1.00 | 17.96 | C |
| ATOM | 1116 | CG | TYR | A | 236 | −2.849 | 2.230 | −38.594 | 1.00 | 18.17 | C |
| ATOM | 1117 | CD1 | TYR | A | 236 | −1.949 | 1.476 | −37.829 | 1.00 | 17.85 | C |
| ATOM | 1118 | CE1 | TRY | A | 236 | −1.509 | 1.920 | −36.595 | 1.00 | 18.22 | C |
| ATOM | 1119 | CZ | TYR | A | 236 | −1.962 | 3.145 | −36.106 | 1.00 | 17.83 | C |
| ATOM | 1120 | OH | TYR | A | 236 | −1.538 | 3.598 | −34.875 | 1.00 | 17.25 | O |
| ATOM | 1121 | CE2 | TYR | A | 236 | −2.841 | 3.914 | −36.847 | 1.00 | 17.33 | C |
| ATOM | 1122 | CD2 | TYR | A | 236 | −3.294 | 3.448 | −38.076 | 1.00 | 17.78 | C |
| ATOM | 1123 | C | TYR | A | 236 | −5.154 | 0.508 | −41.199 | 1.00 | 18.50 | C |
| ATOM | 1124 | O | TYR | A | 236 | −4.503 | −0.137 | −42.022 | 1.00 | 18.26 | O |
| ATOM | 1125 | N | MET | A | 237 | −6.386 | 0.963 | −41.425 | 1.00 | 19.33 | N |
| ATOM | 1126 | CA | MET | A | 237 | −7.226 | 0.489 | −42.523 | 1.00 | 20.35 | C |
| ATOM | 1127 | CB | MET | A | 237 | −8.596 | 0.087 | −41.937 | 1.00 | 20.56 | C |
| ATOM | 1128 | CG | MET | A | 237 | −8.565 | −0.899 | −40.785 | 1.00 | 21.27 | C |
| ATOM | 1129 | SD | MET | A | 237 | −7.793 | −2.476 | −41.176 | 1.00 | 21.56 | S |
| ATOM | 1130 | CE | MET | A | 237 | −8.714 | −3.023 | −42.608 | 1.00 | 21.55 | C |
| ATOM | 1131 | C | MET | A | 237 | −7.408 | 1.472 | −43.691 | 1.00 | 20.84 | C |
| ATOM | 1132 | O | MET | A | 237 | −8.320 | 1.283 | −44.490 | 1.00 | 20.12 | O |
| ATOM | 1133 | N | CYS | A | 238 | −6.560 | 2.493 | −43.765 | 1.00 | 21.91 | N |
| ATOM | 1134 | CA | CYS | A | 238 | −6.628 | 3.508 | −44.837 | 1.00 | 23.03 | C |
| ATOM | 1135 | CB | CYS | A | 238 | −7.700 | 4.593 | −44.545 | 1.00 | 22.44 | C |
| ATOM | 1136 | SG | CYS | A | 238 | −7.993 | 5.859 | −45.840 | 1.00 | 21.63 | S |
| ATOM | 1137 | C | CYS | A | 238 | −5.238 | 4.143 | −44.934 | 1.00 | 25.16 | C |
| ATOM | 1138 | O | CYS | A | 238 | −4.355 | 3.929 | −44.071 | 1.00 | 24.84 | O |
| ATOM | 1139 | N | ASN | A | 239 | −5.017 | 4.859 | −46.025 | 1.00 | 27.80 | N |
| ATOM | 1140 | CA | ASN | A | 239 | −3.671 | 5.426 | −46.359 | 1.00 | 29.56 | C |
| ATOM | 1141 | CB | ASN | A | 239 | −3.211 | 4.962 | −47.757 | 1.00 | 30.95 | C |
| ATOM | 1142 | CG | ASN | A | 239 | −1.862 | 4.252 | −47.784 | 1.00 | 33.14 | C |
| ATOM | 1143 | OD1 | ASN | A | 239 | −0.832 | 4.876 | −48.022 | 1.00 | 35.74 | O |
| ATOM | 1144 | ND2 | ASN | A | 239 | −1.886 | 2.929 | −47.640 | 1.00 | 32.60 | N |
| ATOM | 1145 | C | ASN | A | 239 | −3.845 | 6.927 | −46.400 | 1.00 | 31.26 | C |
| ATOM | 1146 | O | ASN | A | 239 | −4.930 | 7.435 | −46.756 | 1.00 | 31.30 | O |
| ATOM | 1147 | N | SER | A | 240 | −2.791 | 7.671 | −46.076 | 1.00 | 33.40 | N |
| ATOM | 1148 | CA | SER | A | 240 | −2.793 | 9.132 | −46.309 | 1.00 | 33.56 | C |
| ATOM | 1149 | CB | SER | A | 240 | −1.668 | 9.837 | −45.549 | 1.00 | 33.80 | C |
| ATOM | 1150 | OG | SER | A | 240 | −0.400 | 9.393 | −45.988 | 1.00 | 35.45 | O |
| ATOM | 1151 | C | SER | A | 240 | −2.724 | 9.452 | −47.813 | 1.00 | 34.11 | C |
| ATOM | 1152 | O | SER | A | 240 | −3.279 | 10.457 | −48.261 | 1.00 | 35.78 | O |
| ATOM | 1153 | N | SER | A | 241 | −2.059 | 8.584 | −48.578 | 1.00 | 32.84 | N |
| ATOM | 1154 | CA | SER | A | 241 | −1.944 | 8.725 | −50.032 | 1.00 | 32.02 | C |
| ATOM | 1155 | CB | SER | A | 241 | −0.822 | 7.807 | −50.565 | 1.00 | 32.62 | C |
| ATOM | 1156 | OG | SER | A | 241 | −1.186 | 6.437 | −50.483 | 1.00 | 31.61 | O |
| ATOM | 1157 | C | SER | A | 241 | −3.255 | 8.448 | −50.798 | 1.00 | 31.08 | C |
| ATOM | 1158 | O | SER | A | 241 | −3.332 | 8.754 | −51.990 | 1.00 | 31.27 | O |
| ATOM | 1159 | N | CYS | A | 242 | −4.264 | 7.881 | −50.132 | 1.00 | 29.90 | N |
| ATOM | 1160 | CA | CYS | A | 242 | −5.548 | 7.532 | −50.757 | 1.00 | 28.82 | C |
| ATOM | 1161 | CB | CYS | A | 242 | −6.569 | 7.047 | −49.712 | 1.00 | 26.65 | C |
| ATOM | 1162 | SG | CYS | A | 242 | −6.418 | 5.310 | −49.238 | 1.00 | 23.94 | S |
| ATOM | 1163 | C | CYS | A | 242 | −6.185 | 8.659 | −51.562 | 1.00 | 30.92 | C |
| ATOM | 1164 | O | CYS | A | 242 | −6.397 | 9.766 | −51.060 | 1.00 | 29.63 | O |
| ATOM | 1165 | N | MET | A | 243 | −6.484 | 8.346 | −52.823 | 1.00 | 33.11 | N |
| ATOM | 1166 | CA | MET | A | 243 | −7.224 | 9.228 | −53.701 | 1.00 | 35.28 | C |
| ATOM | 1167 | CB | MET | A | 243 | −7.287 | 8.610 | −55.105 | 1.00 | 35.78 | C |
| ATOM | 1168 | CG | MET | A | 243 | −7.649 | 9.564 | −56.226 | 1.00 | 36.89 | C |
| ATOM | 1169 | SD | MET | A | 243 | −7.116 | 8.940 | −57.839 | 1.00 | 37.05 | S |
| ATOM | 1170 | CE | MET | A | 243 | −7.444 | 10.381 | −58.851 | 1.00 | 37.22 | C |
| ATOM | 1171 | C | MET | A | 243 | −8.627 | 9.408 | −53.133 | 1.00 | 37.50 | C |
| ATOM | 1172 | O | MET | A | 243 | −9.277 | 8.428 | −52.756 | 1.00 | 38.40 | O |
| ATOM | 1173 | N | GLY | A | 244 | −9.066 | 10.660 | −53.033 | 1.00 | 39.67 | N |
| ATOM | 1174 | CA | GLY | A | 244 | −10.414 | 10.983 | −52.564 | 1.00 | 40.78 | C |
| ATOM | 1175 | C | GLY | A | 244 | −10.626 | 11.027 | −51.061 | 1.00 | 42.14 | C |
| ATOM | 1176 | O | GLY | A | 244 | −11.767 | 11.146 | −50.613 | 1.00 | 43.53 | O |
| ATOM | 1177 | N | GLY | A | 245 | −9.549 | 10.940 | −50.277 | 1.00 | 44.06 | N |
| ATOM | 1178 | CA | GLY | A | 245 | −9.606 | 11.147 | −48.829 | 1.00 | 46.43 | C |
| ATOM | 1179 | C | GLY | A | 245 | −9.827 | 12.609 | −48.472 | 1.00 | 47.76 | C |
| ATOM | 1180 | O | GLY | A | 245 | −9.832 | 13.477 | −49.355 | 1.00 | 44.96 | O |
| ATOM | 1181 | N | MET | A | 246 | −10.032 | 12.881 | −47.180 | 1.00 | 51.43 | N |
| ATOM | 1182 | CA | MET | A | 246 | −10.296 | 14.255 | −46.714 | 1.00 | 53.85 | C |
| ATOM | 1183 | CB | MET | A | 246 | −10.763 | 14.273 | −45.251 | 1.00 | 59.21 | C |
| ATOM | 1184 | CG | MET | A | 246 | −12.162 | 13.699 | −45.051 | 1.00 | 63.23 | C |
| ATOM | 1185 | SD | MET | A | 246 | −13.482 | 14.759 | −45.633 | 1.00 | 72.68 | S |
| ATOM | 1186 | CE | MET | A | 246 | −13.844 | 13.946 | −47.256 | 1.00 | 72.32 | C |
| ATOM | 1187 | C | MET | A | 246 | −9.086 | 15.161 | −46.934 | 1.00 | 48.85 | C |
| ATOM | 1188 | O | MET | A | 246 | −9.255 | 16.288 | −47.399 | 1.00 | 48.83 | O |
| ATOM | 1189 | N | ASN | A | 247 | −7.889 | 14.653 | −46.647 | 1.00 | 45.96 | N |
| ATOM | 1190 | CA | ASN | A | 247 | −6.646 | 15.348 | −46.992 | 1.00 | 42.48 | C |
| ATOM | 1191 | CB | ASN | A | 247 | −6.409 | 16.511 | −46.020 | 1.00 | 40.25 | C |
| ATOM | 1192 | CG | ASN | A | 247 | −6.290 | 16.055 | −44.585 | 1.00 | 38.81 | C |
| ATOM | 1193 | OD1 | ASN | A | 247 | −5.278 | 15.480 | −44.195 | 1.00 | 37.79 | O |
| ATOM | 1194 | ND2 | ASN | A | 247 | −7.323 | 16.307 | −43.791 | 1.00 | 39.30 | N |
| ATOM | 1195 | C | ASN | A | 247 | −5.447 | 14.401 | −47.006 | 1.00 | 41.68 | C |
| ATOM | 1196 | O | ASN | A | 247 | −5.574 | 13.223 | −46.662 | 1.00 | 40.64 | O |
| ATOM | 1197 | N | ARG | A | 248 | −4.288 | 14.934 | −47.398 | 1.00 | 39.79 | N |
| ATOM | 1198 | CA | ARG | A | 248 | −3.038 | 14.186 | −47.428 | 1.00 | 40.11 | C |
| ATOM | 1199 | CB | ARG | A | 248 | −2.341 | 14.394 | −48.778 | 1.00 | 44.33 | C |
| ATOM | 1200 | CG | ARG | A | 248 | −3.068 | 13.843 | −49.989 | 1.00 | 48.00 | C |
| ATOM | 1201 | CD | ARG | A | 248 | −2.294 | 14.127 | −51.259 | 1.00 | 51.60 | C |
| ATOM | 1202 | NE | ARG | A | 248 | −2.303 | 15.554 | −51.593 | 1.00 | 55.82 | N |
| ATOM | 1203 | CZ | ARG | A | 248 | −1.582 | 16.132 | −52.554 | 1.00 | 57.34 | C |
| ATOM | 1204 | NH1 | ARG | A | 248 | −0.759 | 15.421 | −53.332 | 1.00 | 57.90 | N |
| ATOM | 1205 | NH2 | ARG | A | 248 | −1.683 | 17.449 | −52.743 | 1.00 | 56.05 | N |
| ATOM | 1206 | C | ARG | A | 248 | −2.085 | 14.543 | −46.278 | 1.00 | 36.65 | C |
| ATOM | 1207 | O | ARG | A | 248 | −0.865 | 14.453 | −46.443 | 1.00 | 36.88 | O |
| ATOM | 1208 | N | SER | A | 249 | −2.620 | 14.937 | −45.119 | 1.00 | 32.18 | N |
| ATOM | 1209 | CA | SER | A | 249 | −1.769 | 15.187 | −43.956 | 1.00 | 31.26 | C |
| ATOM | 1210 | CB | SER | A | 249 | −2.521 | 15.973 | −42.876 | 1.00 | 30.21 | C |
| ATOM | 1211 | OG | SER | A | 249 | −1.689 | 16.260 | −41.758 | 1.00 | 29.12 | O |
| ATOM | 1212 | C | SER | A | 249 | −1.282 | 13.846 | −43.367 | 1.00 | 30.26 | C |
| ATOM | 1213 | O | SER | A | 249 | −2.026 | 12.857 | −43.365 | 1.00 | 29.46 | O |
| ATOM | 1214 | N | PRO | A | 250 | −0.039 | 13.816 | −42.849 | 1.00 | 28.82 | N |
| ATOM | 1215 | CA | PRO | A | 250 | 0.371 | 12.737 | −41.952 | 1.00 | 28.41 | C |
| ATOM | 1216 | CB | PRO | A | 250 | 1.807 | 13.119 | −41.553 | 1.00 | 28.74 | C |
| ATOM | 1217 | CG | PRO | A | 250 | 2.270 | 14.016 | −42.643 | 1.00 | 29.53 | C |
| ATOM | 1218 | CD | PRO | A | 250 | 1.052 | 14.786 | −43.063 | 1.00 | 29.91 | C |
| ATOM | 1219 | C | PRO | A | 250 | −0.518 | 12.652 | −40.695 | 1.00 | 26.78 | C |
| ATOM | 1220 | O | PRO | A | 250 | −1.033 | 13.654 | −40.216 | 1.00 | 25.69 | O |
| ATOM | 1221 | N | ILE | A | 251 | −0.679 | 11.441 | −40.194 | 1.00 | 25.14 | N |
| ATOM | 1222 | CA | ILE | A | 251 | −1.565 | 11.114 | −39.096 | 1.00 | 25.01 | C |
| ATOM | 1223 | CB | ILE | A | 251 | −2.392 | 9.852 | −39.471 | 1.00 | 25.97 | C |
| ATOM | 1224 | CG1 | ILE | A | 251 | −3.431 | 10.219 | −40.538 | 1.00 | 26.40 | C |
| ATOM | 1225 | CD1 | ILE | A | 251 | −3.801 | 9.093 | −41.481 | 1.00 | 27.22 | C |
| ATOM | 1226 | CG2 | ILE | A | 251 | −3.124 | 9.269 | −38.285 | 1.00 | 26.20 | C |
| ATOM | 1227 | C | ILE | A | 251 | −0.758 | 10.915 | −37.819 | 1.00 | 23.68 | C |
| ATOM | 1228 | O | ILE | A | 251 | 0.353 | 10.418 | −37.857 | 1.00 | 23.09 | O |
| ATOM | 1229 | N | LEU | A | 252 | −1.337 | 11.302 | −36.691 | 1.00 | 23.00 | N |
| ATOM | 1230 | CA | LEU | A | 252 | −0.762 | 11.100 | −35.352 | 1.00 | 22.49 | C |
| ATOM | 1231 | CB | LEU | A | 252 | −0.684 | 12.419 | −34.589 | 1.00 | 23.99 | C |
| ATOM | 1232 | CG | LEU | A | 252 | 0.496 | 13.328 | −34.258 | 1.00 | 25.78 | C |
| ATOM | 1233 | CD1 | LEU | A | 252 | 0.235 | 13.943 | −32.889 | 1.00 | 25.76 | C |
| ATOM | 1234 | CD2 | LEU | A | 252 | 1.840 | 12.638 | −34.244 | 1.00 | 27.22 | C |
| ATOM | 1235 | C | LEU | A | 252 | −1.724 | 10.186 | −34.602 | 1.00 | 21.12 | C |
| ATOM | 1236 | O | LEU | A | 252 | −2.925 | 10.456 | −34.588 | 1.00 | 21.16 | O |
| ATOM | 1237 | N | THR | A | 253 | −1.206 | 9.122 | −33.994 | 1.00 | 18.99 | N |
| ATOM | 1238 | CA | THR | A | 253 | −1.986 | 8.261 | −33.118 | 1.00 | 18.45 | C |
| ATOM | 1239 | CB | THR | A | 253 | −1.611 | 6.771 | −33.315 | 1.00 | 17.90 | C |
| ATOM | 1240 | OG1 | THR | A | 253 | −1.890 | 6.392 | −34.664 | 1.00 | 16.44 | O |
| ATOM | 1241 | CG2 | THR | A | 253 | −2.399 | 5.873 | −32.377 | 1.00 | 17.59 | C |
| ATOM | 1242 | C | THR | A | 253 | −1.743 | 8.673 | −31.671 | 1.00 | 18.69 | C |
| ATOM | 1243 | O | THR | A | 253 | −0.596 | 8.801 | −31.248 | 1.00 | 19.03 | O |
| ATOM | 1244 | N | ILE | A | 254 | −2.823 | 8.869 | −30.923 | 1.00 | 19.42 | N |
| ATOM | 1245 | CA | ILE | A | 254 | −2.761 | 9.230 | −29.505 | 1.00 | 20.56 | C |
| ATOM | 1246 | CB | ILE | A | 254 | −3.639 | 10.466 | −29.204 | 1.00 | 21.49 | C |
| ATOM | 1247 | CG1 | ILE | A | 254 | −3.099 | 11.695 | −29.947 | 1.00 | 21.61 | C |
| ATOM | 1248 | CD1 | ILE | A | 254 | −4.135 | 12.765 | −30.206 | 1.00 | 21.87 | C |
| ATOM | 1249 | CG2 | ILE | A | 254 | −3.671 | 10.745 | −27.715 | 1.00 | 21.88 | C |
| ATOM | 1250 | C | ILE | A | 254 | −3.274 | 8.056 | −28.696 | 1.00 | 20.95 | C |
| ATOM | 1251 | O | ILE | A | 254 | −4.441 | 7.670 | −28.846 | 1.00 | 20.69 | O |
| ATOM | 1252 | N | ILE | A | 255 | −2.406 | 7.506 | −27.844 | 1.00 | 21.35 | N |
| ATOM | 1253 | CA | ILE | A | 255 | −2.767 | 6.420 | −26.934 | 1.00 | 21.30 | C |
| ATOM | 1254 | CB | ILE | A | 255 | −1.695 | 5.308 | −26.896 | 1.00 | 21.04 | C |
| ATOM | 1255 | CG1 | ILE | A | 255 | −1.352 | 4.806 | −28.302 | 1.00 | 21.19 | C |
| ATOM | 1256 | CD1 | ILE | A | 255 | −2.503 | 4.245 | −29.081 | 1.00 | 21.77 | C |
| ATOM | 1257 | CG2 | ILE | A | 255 | −2.134 | 4.152 | −26.001 | 1.00 | 21.26 | C |
| ATOM | 1258 | C | ILE | A | 255 | −2.914 | 7.035 | −25.550 | 1.00 | 21.30 | C |
| ATOM | 1259 | O | ILE | A | 255 | −1.963 | 7.629 | −25.031 | 1.00 | 21.43 | O |
| ATOM | 1260 | N | THR | A | 256 | −4.099 | 6.890 | −24.957 | 1.00 | 22.18 | N |
| ATOM | 1261 | CA | THR | A | 256 | −4.354 | 7.361 | −23.608 | 1.00 | 23.60 | C |
| ATOM | 1262 | CB | THR | A | 256 | −5.491 | 8.395 | −23.555 | 1.00 | 24.43 | C |
| ATOM | 1263 | OG1 | THR | A | 256 | −6.650 | 7.885 | −24.233 | 1.00 | 25.46 | O |
| ATOM | 1264 | CG2 | THR | A | 256 | −5.074 | 9.696 | −24.221 | 1.00 | 25.22 | C |
| ATOM | 1265 | C | THR | A | 256 | −4.704 | 6.189 | −22.697 | 1.00 | 24.71 | C |
| ATOM | 1266 | O | THR | A | 256 | −5.343 | 5.226 | −23.105 | 1.00 | 23.88 | O |
| ATOM | 1267 | N | LEU | A | 257 | −4.266 | 6.300 | −21.449 | 1.00 | 26.26 | N |
| ATOM | 1268 | CA | LEU | A | 257 | −4.672 | 5.416 | −20.380 | 1.00 | 27.42 | C |
| ATOM | 1269 | CB | LEU | A | 257 | −3.467 | 5.108 | −19.495 | 1.00 | 27.93 | C |
| ATOM | 1270 | CG | LEU | A | 257 | −3.432 | 3.820 | −18.700 | 1.00 | 28.36 | C |
| ATOM | 1271 | CD1 | LEU | A | 257 | −3.411 | 2.608 | −19.611 | 1.00 | 28.19 | C |
| ATOM | 1272 | CD2 | LEU | A | 257 | −2.205 | 3.813 | −17.806 | 1.00 | 29.56 | C |
| ATOM | 1273 | C | LEU | A | 257 | −5.775 | 6.144 | −19.598 | 1.00 | 27.98 | C |
| ATOM | 1274 | O | LEU | A | 257 | −5.589 | 7.285 | −19.177 | 1.00 | 28.90 | O |
| ATOM | 1275 | N | GLU | A | 258 | −6.933 | 5.493 | −19.443 | 1.00 | 29.72 | N |
| ATOM | 1276 | CA | GLU | A | 258 | −8.085 | 6.049 | −18.726 | 1.00 | 31.05 | C |
| ATOM | 1277 | CB | GLU | A | 258 | −9.300 | 6.190 | −19.646 | 1.00 | 33.07 | C |
| ATOM | 1278 | CG | GLU | A | 258 | −9.073 | 6.759 | −21.033 | 1.00 | 34.15 | C |
| ATOM | 1279 | CD | GLU | A | 258 | −10.385 | 6.827 | −21.828 | 1.00 | 35.08 | C |
| ATOM | 1280 | OE1 | GLU | A | 258 | −11.255 | 5.934 | −21.658 | 1.00 | 36.62 | O |
| ATOM | 1281 | OE2 | GLU | A | 258 | −10.561 | 7.787 | −22.616 | 1.00 | 36.41 | O |
| ATOM | 1282 | C | GLU | A | 258 | −8.519 | 5.108 | −17.610 | 1.00 | 30.16 | C |
| ATOM | 1283 | O | GLU | A | 258 | −8.334 | 3.891 | −17.724 | 1.00 | 28.60 | O |
| ATOM | 1284 | N | ASP | A | 259 | −9.160 | 5.662 | −16.571 | 1.00 | 31.62 | N |
| ATOM | 1285 | CA | ASP | A | 259 | −9.848 | 4.834 | −15.565 | 1.00 | 32.75 | C |
| ATOM | 1286 | CB | ASP | A | 259 | −9.941 | 5.545 | −14.193 | 1.00 | 34.55 | C |
| ATOM | 1287 | CG | ASP | A | 259 | −10.904 | 6.742 | −14.164 | 1.00 | 35.90 | C |
| ATOM | 1288 | OD1 | ASP | A | 259 | −11.725 | 6.934 | −15.087 | 1.00 | 38.77 | O |
| ATOM | 1289 | OD2 | ASP | A | 259 | −10.841 | 7.506 | −13.184 | 1.00 | 36.75 | O |
| ATOM | 1290 | C | ASP | A | 259 | −11.214 | 4.372 | −16.091 | 1.00 | 32.02 | C |
| ATOM | 1291 | O | ASP | A | 259 | −11.592 | 4.692 | −17.215 | 1.00 | 30.77 | O |
| ATOM | 1292 | N | SER | A | 260 | −11.950 | 3.609 | −15.284 | 1.00 | 31.82 | N |
| ATOM | 1293 | CA | SER | A | 260 | −13.266 | 3.103 | −15.692 | 1.00 | 31.06 | C |
| ATOM | 1294 | CB | SER | A | 260 | −13.802 | 2.127 | −14.654 | 1.00 | 31.53 | C |
| ATOM | 1295 | OG | SER | A | 260 | −13.761 | 2.693 | −13.346 | 1.00 | 31.51 | O |
| ATOM | 1296 | C | SER | A | 260 | −14.289 | 4.214 | −15.989 | 1.00 | 30.84 | C |
| ATOM | 1297 | O | SER | A | 260 | −15.165 | 4.022 | −16.821 | 1.00 | 30.70 | O |
| ATOM | 1298 | N | SER | A | 261 | −14.155 | 5.363 | −15.329 | 1.00 | 32.82 | N |
| ATOM | 1299 | CA | SER | A | 261 | −15.035 | 6.521 | −15.556 | 1.00 | 32.68 | C |
| ATOM | 1300 | CB | SER | A | 261 | −15.241 | 7.293 | −14.242 | 1.00 | 34.40 | C |
| ATOM | 1301 | OG | SER | A | 261 | −15.594 | 6.421 | −13.178 | 1.00 | 35.76 | O |
| ATOM | 1302 | C | SER | A | 261 | −14.544 | 7.490 | −16.638 | 1.00 | 31.38 | C |
| ATOM | 1303 | O | SER | A | 261 | −15.134 | 8.548 | −16.820 | 1.00 | 31.25 | O |
| ATOM | 1304 | N | GLY | A | 262 | −13.475 | 7.140 | −17.350 | 1.00 | 30.74 | N |
| ATOM | 1305 | CA | GLY | A | 262 | −12.970 | 7.953 | −18.466 | 1.00 | 30.77 | C |
| ATOM | 1306 | C | GLY | A | 262 | −11.994 | 9.077 | −18.139 | 1.00 | 30.89 | C |
| ATOM | 1307 | O | GLY | A | 262 | −11.651 | 9.865 | −19.022 | 1.00 | 31.45 | O |
| ATOM | 1308 | N | ASN | A | 263 | −11.537 | 9.162 | −16.888 | 1.00 | 30.27 | N |
| ATOM | 1309 | CA | ASN | A | 263 | −10.548 | 10.182 | −16.491 | 1.00 | 29.27 | C |
| ATOM | 1310 | CB | ASN | A | 263 | −10.507 | 10.365 | −14.970 | 1.00 | 29.78 | C |
| ATOM | 1311 | CG | ASN | A | 263 | −11.839 | 10.810 | −14.392 | 1.00 | 30.56 | C |
| ATOM | 1312 | OD1 | ASN | A | 263 | −12.304 | 11.922 | −14.643 | 1.00 | 32.61 | O |
| ATOM | 1313 | ND2 | ASN | A | 263 | −12.454 | 9.945 | −13.607 | 1.00 | 30.57 | N |
| ATOM | 1314 | C | ASN | A | 263 | −9.153 | 9.795 | −16.983 | 1.00 | 27.78 | C |
| ATOM | 1315 | O | ASN | A | 263 | −8.765 | 8.634 | −16.914 | 1.00 | 25.68 | O |
| ATOM | 1316 | N | LEU | A | 264 | −8.411 | 10.787 | −17.460 | 1.00 | 27.71 | N |
| ATOM | 1317 | CA | LEU | A | 264 | −7.070 | 10.603 | −18.006 | 1.00 | 28.68 | C |
| ATOM | 1318 | CB | LEU | A | 264 | −6.627 | 11.921 | −18.665 | 1.00 | 29.97 | C |
| ATOM | 1319 | CG | LEU | A | 264 | −5.213 | 12.012 | −19.225 | 1.00 | 29.71 | C |
| ATOM | 1320 | CD1 | LEU | A | 264 | −5.213 | 11.242 | −20.525 | 1.00 | 29.46 | C |
| ATOM | 1321 | CD2 | LEU | A | 264 | −4.798 | 13.470 | −19.417 | 1.00 | 29.27 | C |
| ATOM | 1322 | C | LEU | A | 264 | −6.047 | 10.230 | −16.932 | 1.00 | 28.99 | C |
| ATOM | 1323 | O | LEU | A | 264 | −5.907 | 10.930 | −15.936 | 1.00 | 30.16 | O |
| ATOM | 1324 | N | LEU | A | 265 | −5.347 | 9.118 | −17.148 | 1.00 | 28.04 | N |
| ATOM | 1325 | CA | LEU | A | 265 | −4.239 | 8.682 | −16.306 | 1.00 | 27.19 | C |
| ATOM | 1326 | CB | LEU | A | 265 | −4.394 | 7.195 | −15.975 | 1.00 | 28.04 | C |
| ATOM | 1327 | CG | LEU | A | 265 | −5.746 | 6.779 | −15.393 | 1.00 | 28.61 | C |
| ATOM | 1328 | CD1 | LEU | A | 265 | −5.739 | 5.301 | −15.028 | 1.00 | 28.38 | C |
| ATOM | 1329 | CD2 | LEU | A | 265 | −6.126 | 7.602 | −14.170 | 1.00 | 28.97 | C |
| ATOM | 1330 | C | LEU | A | 265 | −2.874 | 8.916 | −16.969 | 1.00 | 26.68 | C |
| ATOM | 1331 | O | LEU | A | 265 | −1.874 | 9.134 | −16.279 | 1.00 | 25.86 | O |
| ATOM | 1332 | N | GLY | A | 266 | −2.821 | 8.851 | −18.299 | 1.00 | 26.63 | N |
| ATOM | 1333 | CA | GLY | A | 266 | −1.578 | 8.996 | −19.030 | 1.00 | 25.91 | C |
| ATOM | 1334 | C | GLY | A | 266 | −1.785 | 9.113 | −20.526 | 1.00 | 25.22 | C |
| ATOM | 1335 | O | GLY | A | 266 | −2.818 | 8.709 | −21.039 | 1.00 | 25.64 | O |
| ATOM | 1336 | N | ARG | A | 267 | −0.790 | 9.660 | −21.219 | 1.00 | 24.01 | N |
| ATOM | 1337 | CA | ARG | A | 267 | −0.865 | 9.891 | −22.651 | 1.00 | 23.97 | C |
| ATOM | 1338 | CB | ARG | A | 267 | −1.527 | 11.251 | −22.962 | 1.00 | 24.52 | C |
| ATOM | 1339 | CG | ARG | A | 267 | −1.861 | 11.440 | −24.442 | 1.00 | 24.78 | C |
| ATOM | 1340 | CD | ARG | A | 267 | −2.205 | 12.857 | −24.842 | 1.00 | 24.77 | C |
| ATOM | 1341 | NE | ARG | A | 267 | −3.465 | 13.317 | −24.261 | 1.00 | 25.47 | N |
| ATOM | 1342 | CZ | ARG | A | 267 | −3.610 | 14.215 | −23.283 | 1.00 | 26.41 | C |
| ATOM | 1343 | NH1 | ARG | A | 267 | −2.565 | 14.794 | −22.687 | 1.00 | 26.67 | N |
| ATOM | 1344 | NH2 | ARG | A | 267 | −4.840 | 14.532 | −22.878 | 1.00 | 26.42 | N |
| ATOM | 1345 | C | ARG | A | 267 | 0.511 | 9.824 | −23.313 | 1.00 | 22.68 | C |
| ATOM | 1346 | O | ARG | A | 267 | 1.502 | 10.318 | −22.749 | 1.00 | 22.90 | O |
| ATOM | 1347 | N | ASN | A | 268 | 0.543 | 9.220 | −24.502 | 1.00 | 21.12 | N |
| ATOM | 1348 | CA | ASN | A | 268 | 1.684 | 9.328 | −25.404 | 1.00 | 20.54 | C |
| ATOM | 1349 | CB | ASN | A | 268 | 2.670 | 8.163 | −25.212 | 1.00 | 22.08 | C |
| ATOM | 1350 | CG | ASN | A | 268 | 4.182 | 8.579 | −25.336 | 1.00 | 23.95 | C |
| ATOM | 1351 | OD1 | ASN | A | 268 | 4.614 | 9.446 | −26.145 | 1.00 | 22.90 | O |
| ATOM | 1352 | ND2 | ASN | A | 268 | 4.999 | 7.894 | −24.544 | 1.00 | 25.44 | N |
| ATOM | 1353 | C | ASN | A | 268 | 1.159 | 9.349 | −26.829 | 1.00 | 19.15 | C |
| ATOM | 1354 | O | ASN | A | 268 | −0.032 | 9.208 | −27.069 | 1.00 | 19.24 | O |
| ATOM | 1355 | N | SER | A | 269 | 2.056 | 9.529 | −27.782 | 1.00 | 18.18 | N |
| ATOM | 1356 | CA | SER | A | 269 | 1.665 | 9.600 | −29.177 | 1.00 | 17.51 | C |
| ATOM | 1357 | CB | SER | A | 269 | 1.075 | 10.985 | −29.483 | 1.00 | 17.28 | C |
| ATOM | 1358 | OG | SER | A | 269 | 2.033 | 11.993 | −29.246 | 1.00 | 16.66 | O |
| ATOM | 1359 | C | SER | A | 269 | 2.817 | 9.315 | −30.120 | 1.00 | 17.02 | C |
| ATOM | 1360 | O | SER | A | 269 | 3.980 | 9.404 | −29.753 | 1.00 | 16.19 | O |
| ATOM | 1361 | N | SER | A | 270 | 2.454 | 8.983 | −31.348 | 1.00 | 17.54 | N |
| ATOM | 1362 | CA | PHE | A | 270 | 3.405 | 8.735 | −32.409 | 1.00 | 17.92 | C |
| ATOM | 1363 | CB | PHE | A | 270 | 3.979 | 7.307 | −32.320 | 1.00 | 17.75 | C |
| ATOM | 1364 | CG | PHE | A | 270 | 2.941 | 6.214 | −32.370 | 1.00 | 17.78 | C |
| ATOM | 1365 | CD1 | PHE | A | 270 | 2.260 | 5.819 | −31.218 | 1.00 | 18.10 | C |
| ATOM | 1366 | CE1 | PHE | A | 270 | 1.321 | 4.790 | −31.261 | 1.00 | 17.94 | C |
| ATOM | 1367 | CZ | PHE | A | 270 | 1.058 | 4.134 | −32.442 | 1.00 | 17.65 | C |
| ATOM | 1368 | CE2 | PHE | A | 270 | 1.733 | 4.510 | −33.602 | 1.00 | 17.62 | C |
| ATOM | 1369 | CD2 | PHE | A | 270 | 2.669 | 5.542 | −33.560 | 1.00 | 17.90 | C |
| ATOM | 1370 | C | PHE | A | 270 | 2.775 | 8.986 | −33.768 | 1.00 | 18.65 | C |
| ATOM | 1371 | O | PHE | A | 270 | 1.573 | 8.856 | −33.950 | 1.00 | 19.21 | O |
| ATOM | 1372 | N | GLU | A | 271 | 3.612 | 9.374 | −34.714 | 1.00 | 19.61 | N |
| ATOM | 1373 | CA | GLU | A | 271 | 3.205 | 9.591 | −36.088 | 1.00 | 20.27 | C |
| ATOM | 1374 | CB | GLU | A | 271 | 4.248 | 10.454 | −36.790 | 1.00 | 21.41 | C |
| ATOM | 1375 | CG | GLU | A | 271 | 3.823 | 11.002 | −38.139 | 1.00 | 22.84 | C |
| ATOM | 1376 | CD | GLU | A | 271 | 4.745 | 12.074 | −38.666 | 1.00 | 24.65 | C |
| ATOM | 1377 | OE1 | GLU | A | 271 | 5.192 | 12.927 | −37.861 | 1.00 | 25.18 | O |
| ATOM | 1378 | OE2 | GLU | A | 271 | 4.955 | 12.127 | −39.896 | 1.00 | 26.46 | O |
| ATOM | 1379 | C | GLU | A | 271 | 3.047 | 8.226 | −36.745 | 1.00 | 20.56 | C |
| ATOM | 1380 | O | GLU | A | 271 | 3.729 | 7.276 | −36.374 | 1.00 | 18.91 | O |
| ATOM | 1381 | N | VAL | A | 272 | 2.138 | 8.131 | −37.704 | 1.00 | 21.87 | N |
| ATOM | 1382 | CA | VAL | A | 272 | 1.918 | 6.879 | −38.432 | 1.00 | 22.31 | C |
| ATOM | 1383 | CB | VAL | A | 272 | 0.649 | 6.135 | −37.937 | 1.00 | 23.22 | C |
| ATOM | 1384 | CG1 | VAL | A | 272 | 0.549 | 4.758 | −38.592 | 1.00 | 23.69 | C |
| ATOM | 1385 | CG2 | VAL | A | 272 | 0.612 | 5.970 | −36.434 | 1.00 | 23.81 | C |
| ATOM | 1386 | C | VAL | A | 272 | 1.853 | 7.162 | −39.945 | 1.00 | 22.53 | C |
| ATOM | 1387 | O | VAL | A | 272 | 1.288 | 8.163 | −40.388 | 1.00 | 23.10 | O |
| ATOM | 1388 | N | ARG | A | 273 | 2.479 | 6.268 | −40.705 | 1.00 | 22.26 | N |
| ATOM | 1389 | CA | ARG | A | 273 | 2.419 | 6.262 | −42.155 | 1.00 | 21.92 | C |
| ATOM | 1390 | CB | ARG | A | 273 | 3.746 | 6.695 | −42.773 | 1.00 | 22.94 | C |
| ATOM | 1391 | CG | ARG | A | 273 | 3.708 | 6.700 | −44.295 | 1.00 | 23.87 | C |
| ATOM | 1392 | CD | ARG | A | 273 | 5.039 | 7.011 | −44.934 | 1.00 | 25.11 | C |
| ATOM | 1393 | NE | ARG | A | 273 | 4.991 | 6.728 | −46.361 | 1.00 | 27.23 | N |
| ATOM | 1394 | CZ | ARG | A | 273 | 4.412 | 7.503 | −47.286 | 1.00 | 29.33 | C |
| ATOM | 1395 | NH1 | ARG | A | 273 | 3.804 | 8.655 | −46.952 | 1.00 | 30.51 | N |
| ATOM | 1396 | NH2 | ARG | A | 273 | 4.424 | 7.127 | −48.567 | 1.00 | 29.54 | N |
| ATOM | 1397 | C | ARG | A | 273 | 2.092 | 4.846 | −42.609 | 1.00 | 21.43 | C |
| ATOM | 1398 | O | ARG | A | 273 | 2.833 | 3.903 | −42.319 | 1.00 | 19.32 | O |
| ATOM | 1399 | N | VAL | A | 274 | 0.971 | 4.719 | −43.315 | 1.00 | 21.50 | N |
| ATOM | 1400 | CA | VAL | A | 274 | 0.572 | 3.468 | −43.938 | 1.00 | 22.19 | C |
| ATOM | 1401 | CB | VAL | A | 274 | −0.963 | 3.314 | −43.936 | 1.00 | 22.31 | C |
| ATOM | 1402 | CG1 | VAL | A | 274 | −1.355 | 1.946 | −44.478 | 1.00 | 22.71 | C |
| ATOM | 1403 | CG2 | VAL | A | 274 | −1.523 | 3.551 | −42.539 | 1.00 | 22.06 | C |
| ATOM | 1404 | C | VAL | A | 274 | 1.124 | 3.492 | −45.362 | 1.00 | 22.09 | C |
| ATOM | 1405 | O | VAL | A | 274 | 0.894 | 4.457 | −46.091 | 1.00 | 22.71 | O |
| ATOM | 1406 | N | CYS | A | 275 | 1.863 | 2.448 | −45.739 | 1.00 | 21.59 | N |
| ATOM | 1407 | CA | CYS | A | 275 | 2.578 | 2.405 | −47.020 | 1.00 | 21.59 | C |
| ATOM | 1408 | CB | CYS | A | 275 | 3.866 | 3.232 | −46.935 | 1.00 | 21.70 | C |
| ATOM | 1409 | SG | CYS | A | 275 | 4.959 | 2.817 | −45.542 | 1.00 | 21.33 | S |
| ATOM | 1410 | C | CYS | A | 275 | 2.908 | 0.974 | −47.455 | 1.00 | 22.35 | C |
| ATOM | 1411 | O | CYS | A | 275 | 2.772 | 0.034 | −46.681 | 1.00 | 22.18 | O |
| ATOM | 1412 | N | ALA | A | 276 | 3.346 | 0.833 | −48.705 | 1.00 | 23.10 | N |
| ATOM | 1413 | CA | ALA | A | 276 | 3.651 | −0.457 | −49.306 | 1.00 | 23.01 | C |
| ATOM | 1414 | CB | ALA | A | 276 | 3.784 | −0.320 | −50.814 | 1.00 | 23.18 | C |
| ATOM | 1415 | C | ALA | A | 276 | 4.907 | −1.130 | −48.740 | 1.00 | 23.73 | C |
| ATOM | 1416 | O | ALA | A | 276 | 4.892 | −2.331 | −48.449 | 1.00 | 23.39 | O |
| ATOM | 1417 | N | ACYS | A | 277 | 5.981 | −0.354 | −48.594 | 0.50 | 24.42 | N |
| ATOM | 1418 | N | BCYS | A | 277 | 5.983 | −0.355 | −48.596 | 0.50 | 23.60 | N |
| ATOM | 1419 | CA | ACYS | A | 277 | 7.276 | −0.867 | −48.132 | 0.50 | 25.15 | C |
| ATOM | 1420 | CA | BCYS | A | 277 | 7.278 | −0.876 | −48.127 | 0.50 | 23.82 | C |
| ATOM | 1421 | CB | ACYS | A | 277 | 8.294 | −0.779 | −49.276 | 0.50 | 25.86 | C |
| ATOM | 1422 | CB | BCYS | A | 277 | 8.316 | −0.857 | −49.262 | 0.50 | 23.66 | C |
| ATOM | 1423 | SG | ACYS | A | 277 | 7.764 | −1.440 | −50.872 | 0.50 | 27.30 | S |
| ATOM | 1424 | SG | BCYS | A | 277 | 9.717 | −1.973 | −48.966 | 0.50 | 22.98 | S |
| ATOM | 1425 | C | ACYS | A | 277 | 7.793 | −0.076 | −46.918 | 0.50 | 24.78 | C |
| ATOM | 1426 | C | BCYS | A | 277 | 7.794 | −0.077 | −46.918 | 0.50 | 24.03 | C |
| ATOM | 1427 | O | ACYS | A | 277 | 8.586 | 0.850 | −47.081 | 0.50 | 25.06 | O |
| ATOM | 1428 | O | BCYS | A | 277 | 8.586 | 0.850 | −47.082 | 0.50 | 24.33 | O |
| ATOM | 1429 | N | PRO | A | 278 | 7.329 | −0.426 | −45.697 | 1.00 | 24.60 | N |
| ATOM | 1430 | CA | PRO | A | 278 | 7.754 | 0.255 | −44.453 | 1.00 | 24.60 | C |
| ATOM | 1431 | CB | PRO | A | 278 | 7.139 | −0.610 | −43.355 | 1.00 | 24.56 | C |
| ATOM | 1432 | CG | PRO | A | 278 | 5.938 | −1.217 | −43.985 | 1.00 | 24.82 | C |
| ATOM | 1433 | CD | PRO | A | 278 | 6.224 | −1.371 | −45.438 | 1.00 | 24.57 | C |
| ATOM | 1434 | C | PRO | A | 278 | 9.260 | 0.378 | −44.245 | 1.00 | 24.55 | C |
| ATOM | 1435 | O | PRO | A | 278 | 9.746 | 1.465 | −43.920 | 1.00 | 24.53 | O |
| ATOM | 1436 | N | GLY | A | 279 | 9.979 | −0.720 | −44.431 | 1.00 | 24.69 | N |
| ATOM | 1437 | CA | GLY | A | 279 | 11.449 | −0.733 | −44.292 | 1.00 | 26.38 | C |
| ATOM | 1438 | C | GLY | A | 279 | 12.121 | 0.315 | −45.151 | 1.00 | 27.10 | C |
| ATOM | 1439 | O | GLY | A | 279 | 12.883 | 1.132 | −44.645 | 1.00 | 26.46 | O |
| ATOM | 1440 | N | ARG | A | 280 | 11.806 | 0.312 | −46.446 | 1.00 | 29.51 | N |
| ATOM | 1441 | CA | ARG | A | 280 | 12.418 | 1.239 | −47.396 | 1.00 | 33.24 | C |
| ATOM | 1442 | CB | ARG | A | 280 | 12.040 | 0.846 | −48.832 | 1.00 | 37.45 | C |
| ATOM | 1443 | CG | ARG | A | 280 | 12.605 | 1.677 | −49.959 | 1.00 | 41.33 | C |
| ATOM | 1444 | CD | ARG | A | 280 | 11.917 | 1.296 | −51.275 | 1.00 | 43.73 | C |
| ATOM | 1445 | NE | ARG | A | 280 | 10.668 | 2.041 | −51.493 | 1.00 | 45.13 | N |
| ATOM | 1446 | CZ | ARG | A | 280 | 9.830 | 1.870 | −52.524 | 1.00 | 47.53 | C |
| ATOM | 1447 | NH1 | ARG | A | 280 | 10.052 | 0.946 | −53.468 | 1.00 | 49.13 | N |
| ATOM | 1448 | NH2 | ARG | A | 280 | 8.736 | 2.627 | −52.615 | 1.00 | 48.55 | N |
| ATOM | 1449 | C | ARG | A | 280 | 12.019 | 2.683 | −47.096 | 1.00 | 33.35 | C |
| ATOM | 1450 | O | ARG | A | 280 | 12.869 | 3.569 | −47.072 | 1.00 | 35.24 | O |
| ATOM | 1451 | N | ASP | A | 281 | 10.730 | 2.912 | −46.858 | 1.00 | 32.11 | N |
| ATOM | 1452 | CA | ASP | A | 281 | 10.228 | 4.251 | −46.533 | 1.00 | 31.62 | C |
| ATOM | 1453 | CB | ASP | A | 281 | 8.693 | 4.282 | −46.513 | 1.00 | 32.46 | C |
| ATOM | 1454 | CG | ASP | A | 281 | 8.071 | 4.242 | −47.920 | 1.00 | 33.02 | C |
| ATOM | 1455 | OD1 | ASP | A | 281 | 8.791 | 4.350 | −48.935 | 1.00 | 33.66 | O |
| ATOM | 1456 | OD2 | ASP | A | 281 | 6.833 | 4.129 | −48.001 | 1.00 | 33.50 | O |
| ATOM | 1457 | C | ASP | A | 281 | 10.785 | 4.800 | −45.217 | 1.00 | 29.99 | C |
| ATOM | 1458 | O | ASP | A | 281 | 11.019 | 6.006 | −45.116 | 1.00 | 29.98 | O |
| ATOM | 1459 | N | ARG | A | 282 | 11.005 | 3.939 | −44.220 | 1.00 | 26.69 | N |
| ATOM | 1460 | CA | ARG | A | 282 | 11.694 | 4.364 | −42.995 | 1.00 | 25.35 | C |
| ATOM | 1461 | CB | ARG | A | 282 | 11.661 | 3.289 | −41.897 | 1.00 | 24.14 | C |
| ATOM | 1462 | CG | ARG | A | 282 | 12.457 | 3.677 | −40.654 | 1.00 | 22.85 | C |
| ATOM | 1463 | CD | ARG | A | 282 | 12.359 | 2.679 | −39.507 | 1.00 | 22.06 | C |
| ATOM | 1464 | NE | ARG | A | 282 | 13.448 | 2.919 | −38.556 | 1.00 | 21.11 | N |
| ATOM | 1465 | CZ | ARG | A | 282 | 13.944 | 2.033 | −37.691 | 1.00 | 20.96 | C |
| ATOM | 1466 | NH1 | ARG | A | 282 | 13.461 | 0.787 | −37.592 | 1.00 | 21.02 | N |
| ATOM | 1467 | NH2 | ARG | A | 282 | 14.936 | 2.412 | −36.900 | 1.00 | 20.63 | N |
| ATOM | 1468 | C | ARG | A | 282 | 13.137 | 4.750 | −43.310 | 1.00 | 25.20 | C |
| ATOM | 1469 | O | ARG | A | 282 | 13.586 | 5.837 | −42.932 | 1.00 | 25.23 | O |
| ATOM | 1470 | N | ARG | A | 283 | 13.850 | 3.864 | −44.006 | 1.00 | 25.43 | N |
| ATOM | 1471 | CA | ARG | A | 283 | 15.259 | 4.103 | −44.358 | 1.00 | 26.02 | C |
| ATOM | 1472 | CB | ARG | A | 283 | 15.868 | 2.896 | −45.088 | 1.00 | 25.27 | C |
| ATOM | 1473 | CG | ARG | A | 283 | 17.395 | 2.870 | −45.039 | 1.00 | 25.01 | C |
| ATOM | 1474 | CD | ARG | A | 283 | 17.979 | 1.669 | −45.751 | 1.00 | 25.18 | C |
| ATOM | 1475 | NE | ARG | A | 283 | 17.600 | 0.390 | −45.144 | 1.00 | 24.81 | N |
| ATOM | 1476 | CZ | ARG | A | 283 | 17.894 | −0.814 | −45.653 | 1.00 | 24.66 | C |
| ATOM | 1477 | NH1 | ARG | A | 283 | 17.498 | −1.913 | −45.028 | 1.00 | 24.24 | N |
| ATOM | 1478 | NH2 | ARG | A | 283 | 18.584 | −0.934 | −46.784 | 1.00 | 25.18 | N |
| ATOM | 1479 | C | ARG | A | 283 | 15.450 | 5.385 | −45.185 | 1.00 | 27.42 | C |
| ATOM | 1480 | O | ARG | A | 283 | 16.434 | 6.101 | −44.989 | 1.00 | 28.51 | O |
| ATOM | 1481 | N | THR | A | 284 | 14.507 | 5.671 | −46.080 | 1.00 | 27.22 | N |
| ATOM | 1482 | CA | THR | A | 284 | 14.535 | 6.885 | −46.900 | 1.00 | 28.46 | C |
| ATOM | 1483 | CB | THR | A | 284 | 13.504 | 6.813 | −48.048 | 1.00 | 27.97 | C |
| ATOM | 1484 | OG1 | THR | A | 284 | 13.720 | 5.614 | −48.800 | 1.00 | 28.85 | O |
| ATOM | 1485 | CG2 | THR | A | 284 | 13.628 | 8.017 | −48.965 | 1.00 | 27.69 | C |
| ATOM | 1486 | C | THR | A | 284 | 14.278 | 8.155 | −46.087 | 1.00 | 29.16 | C |
| ATOM | 1487 | O | THR | A | 284 | 15.020 | 9.116 | −46.211 | 1.00 | 28.66 | O |
| ATOM | 1488 | N | GLU | A | 285 | 13.231 | 8.160 | −45.263 | 1.00 | 31.07 | N |
| ATOM | 1489 | CA | GLU | A | 285 | 12.930 | 9.328 | −44.412 | 1.00 | 31.31 | C |
| ATOM | 1490 | CB | GLU | A | 285 | 11.563 | 9.204 | −43.733 | 1.00 | 31.04 | C |
| ATOM | 1491 | CG | GLU | A | 285 | 10.386 | 9.349 | −44.688 | 1.00 | 30.29 | C |
| ATOM | 1492 | CD | GLU | A | 285 | 9.037 | 9.456 | −43.995 | 1.00 | 30.69 | C |
| ATOM | 1493 | OE1 | GLU | A | 285 | 8.969 | 9.880 | −42.817 | 1.00 | 31.46 | O |
| ATOM | 1494 | OE2 | GLU | A | 285 | 8.022 | 9.137 | −44.639 | 1.00 | 30.09 | O |
| ATOM | 1495 | C | GLU | A | 285 | 14.009 | 9.606 | −43.360 | 1.00 | 32.94 | C |
| ATOM | 1496 | O | GLU | A | 285 | 14.200 | 10.765 | −42.964 | 1.00 | 34.90 | O |
| ATOM | 1497 | N | GLU | A | 286 | 14.709 | 8.560 | −42.920 | 1.00 | 34.55 | N |
| ATOM | 1498 | CA | GLU | A | 286 | 15.818 | 8.709 | −41.968 | 1.00 | 36.78 | C |
| ATOM | 1499 | CB | GLU | A | 286 | 16.074 | 7.391 | −41.222 | 1.00 | 37.10 | C |
| ATOM | 1500 | CG | GLU | A | 286 | 15.010 | 7.096 | −40.171 | 1.00 | 36.94 | C |
| ATOM | 1501 | CD | GLU | A | 286 | 15.239 | 5.797 | −39.410 | 1.00 | 36.38 | C |
| ATOM | 1502 | OE1 | GLU | A | 286 | 16.198 | 5.056 | −39.708 | 1.00 | 35.96 | O |
| ATOM | 1503 | OE2 | GLU | A | 286 | 14.443 | 5.505 | −38.494 | 1.00 | 33.91 | O |
| ATOM | 1504 | C | GLU | A | 286 | 17.112 | 9.234 | −42.612 | 1.00 | 40.81 | C |
| ATOM | 1505 | O | GLU | A | 286 | 17.948 | 9.825 | −41.943 | 1.00 | 40.89 | O |
| ATOM | 1506 | N | GLU | A | 287 | 17.284 | 8.985 | −43.909 | 1.00 | 46.04 | N |
| ATOM | 1507 | CA | GLU | A | 287 | 18.376 | 9.549 | −44.709 | 1.00 | 50.94 | C |
| ATOM | 1508 | CB | GLU | A | 287 | 18.400 | 8.821 | −46.067 | 1.00 | 51.89 | C |
| ATOM | 1509 | CG | GLU | A | 287 | 19.429 | 9.260 | −47.096 | 1.00 | 52.41 | C |
| ATOM | 1510 | CD | GLU | A | 287 | 19.292 | 8.468 | −48.398 | 1.00 | 54.53 | C |
| ATOM | 1511 | OE1 | GLU | A | 287 | 18.147 | 8.071 | −48.733 | 1.00 | 53.02 | O |
| ATOM | 1512 | OE2 | GLU | A | 287 | 20.316 | 8.238 | −49.103 | 1.00 | 56.49 | O |
| ATOM | 1513 | C | GLU | A | 287 | 18.207 | 11.071 | −44.893 | 1.00 | 54.76 | C |
| ATOM | 1514 | O | GLU | A | 287 | 19.196 | 11.810 | −44.865 | 1.00 | 54.75 | O |
| ATOM | 1515 | N | ASN | A | 288 | 16.951 | 11.521 | −45.093 | 1.00 | 61.25 | N |
| ATOM | 1516 | CA | ASN | A | 288 | 16.671 | 12.934 | −45.371 | 1.00 | 66.82 | C |
| ATOM | 1517 | CB | ASN | A | 288 | 15.472 | 13.127 | −46.334 | 1.00 | 67.76 | C |
| ATOM | 1518 | CG | ASN | A | 288 | 15.788 | 12.651 | −47.755 | 1.00 | 69.51 | C |
| ATOM | 1519 | OD1 | ASN | A | 288 | 16.909 | 12.841 | −48.230 | 1.00 | 69.75 | O |
| ATOM | 1520 | ND2 | ASN | A | 288 | 14.804 | 12.049 | −48.443 | 1.00 | 70.40 | N |
| ATOM | 1521 | C | ASN | A | 288 | 16.528 | 13.802 | −44.138 | 1.00 | 70.09 | C |
| ATOM | 1522 | O | ASN | A | 288 | 16.635 | 15.013 | −44.280 | 1.00 | 71.46 | O |
| ATOM | 1523 | N | LEU | A | 289 | 16.318 | 13.247 | −42.941 | 1.00 | 72.97 | N |
| ATOM | 1524 | CA | LEU | A | 289 | 16.353 | 14.053 | −41.701 | 1.00 | 72.29 | C |
| ATOM | 1525 | CB | LEU | A | 289 | 15.817 | 13.273 | −40.493 | 1.00 | 70.91 | C |
| ATOM | 1526 | CG | LEU | A | 289 | 16.569 | 12.060 | −39.911 | 1.00 | 69.78 | C |
| ATOM | 1527 | CD1 | LEU | A | 289 | 17.690 | 12.431 | −38.946 | 1.00 | 70.60 | C |
| ATOM | 1528 | CD2 | LEU | A | 289 | 15.609 | 11.127 | −39.181 | 1.00 | 69.87 | C |
| ATOM | 1529 | C | LEU | A | 289 | 17.757 | 14.591 | −41.417 | 1.00 | 73.50 | C |
| ATOM | 1530 | O | LEU | A | 289 | 17.917 | 15.732 | −40.957 | 1.00 | 74.11 | O |
| ATOM | 1531 | N | ARG | A | 290 | 18.770 | 13.774 | −41.722 | 1.00 | 74.12 | N |
| ATOM | 1532 | CA | ARG | A | 290 | 20.173 | 14.139 | −41.495 | 1.00 | 75.73 | C |
| ATOM | 1533 | CB | ARG | A | 290 | 21.057 | 12.886 | −41.464 | 1.00 | 76.82 | C |
| ATOM | 1534 | CG | ARG | A | 290 | 22.391 | 13.081 | −40.764 | 1.00 | 79.71 | C |
| ATOM | 1535 | CD | ARG | A | 290 | 23.499 | 12.136 | −41.235 | 1.00 | 80.88 | C |
| ATOM | 1536 | NE | ARG | A | 290 | 23.600 | 11.974 | −42.692 | 1.00 | 82.79 | N |
| ATOM | 1537 | CZ | ARG | A | 290 | 24.001 | 12.906 | −43.564 | 1.00 | 81.77 | C |
| ATOM | 1538 | NH1 | ARG | A | 290 | 24.365 | 14.127 | −43.172 | 1.00 | 82.87 | N |
| ATOM | 1539 | NH2 | ARG | A | 290 | 24.037 | 12.601 | −44.861 | 1.00 | 81.07 | N |
| ATOM | 1540 | C | ARG | A | 290 | 20.658 | 15.123 | −42.572 | 1.00 | 76.54 | C |
| ATOM | 1541 | O | ARG | A | 290 | 21.714 | 15.746 | −42.438 | 1.00 | 75.81 | O |
| TER | 1542 | | ARG | A | 290 | | | | | | |
| ATOM | 1543 | N | SER | B | 96 | 20.741 | −20.243 | −33.611 | 1.00 | 48.77 | N |
| ATOM | 1544 | CA | SER | B | 96 | 22.098 | −20.595 | −34.123 | 1.00 | 48.58 | C |
| ATOM | 1545 | CB | SER | B | 96 | 22.041 | −20.946 | −35.618 | 1.00 | 50.80 | C |
| ATOM | 1546 | OG | SER | B | 96 | 23.325 | −21.281 | −36.115 | 1.00 | 50.44 | O |
| ATOM | 1547 | C | SER | B | 96 | 23.092 | −19.455 | −33.883 | 1.00 | 47.11 | C |
| ATOM | 1548 | O | SER | B | 96 | 22.744 | −18.279 | −34.019 | 1.00 | 49.92 | O |
| ATOM | 1549 | N | VAL | B | 97 | 24.329 | −19.823 | −33.567 | 1.00 | 42.83 | N |
| ATOM | 1550 | CA | VAL | B | 97 | 25.393 | −18.907 | −33.192 | 1.00 | 38.81 | C |
| ATOM | 1551 | CB | VAL | B | 97 | 26.547 | −19.631 | −32.454 | 1.00 | 38.63 | C |
| ATOM | 1552 | CG1 | VAL | B | 97 | 27.613 | −18.622 | −32.028 | 1.00 | 38.60 | C |
| ATOM | 1553 | CG2 | VAL | B | 97 | 26.034 | −20.362 | −31.233 | 1.00 | 38.94 | C |
| ATOM | 1554 | C | VAL | B | 97 | 25.958 | −18.220 | −34.450 | 1.00 | 37.21 | C |
| ATOM | 1555 | O | VAL | B | 97 | 26.491 | −18.926 | −35.290 | 1.00 | 37.47 | O |
| ATOM | 1556 | N | PRO | B | 98 | 25.873 | −16.868 | −34.547 | 1.00 | 33.98 | N |
| ATOM | 1557 | CA | PRO | B | 98 | 26.281 | −16.180 | −35.755 | 1.00 | 32.85 | C |
| ATOM | 1558 | CB | PRO | B | 98 | 26.025 | −14.679 | −35.451 | 1.00 | 33.34 | C |
| ATOM | 1559 | CG | PRO | B | 98 | 25.379 | −14.608 | −34.133 | 1.00 | 34.19 | C |
| ATOM | 1560 | CD | PRO | B | 98 | 25.381 | −15.952 | −33.498 | 1.00 | 34.51 | C |
| ATOM | 1561 | C | PRO | B | 98 | 27.753 | −16.373 | −36.072 | 1.00 | 32.50 | C |
| ATOM | 1562 | O | PRO | B | 98 | 28.566 | −16.454 | −35.139 | 1.00 | 31.46 | O |
| ATOM | 1563 | N | SER | B | 99 | 28.081 | −16.495 | −37.361 | 1.00 | 31.69 | N |
| ATOM | 1564 | CA | SER | B | 99 | 29.442 | −16.811 | −37.763 | 1.00 | 32.51 | C |
| ATOM | 1565 | CB | SER | B | 99 | 29.525 | −17.100 | −39.275 | 1.00 | 33.45 | C |
| ATOM | 1566 | OG | SER | B | 99 | 30.886 | −17.157 | −39.706 | 1.00 | 34.16 | O |
| ATOM | 1567 | C | SER | B | 99 | 30.377 | −15.668 | −37.431 | 1.00 | 32.96 | C |
| ATOM | 1568 | O | SER | B | 99 | 30.001 | −14.493 | −37.547 | 1.00 | 33.62 | O |
| ATOM | 1569 | N | GLN | B | 100 | 31.591 | −16.016 | −37.018 | 1.00 | 33.19 | N |
| ATOM | 1570 | CA | GLN | B | 100 | 32.644 | −15.041 | −36.729 | 1.00 | 34.46 | C |
| ATOM | 1571 | CB | GLN | B | 100 | 33.038 | −15.130 | −35.253 | 1.00 | 35.45 | C |
| ATOM | 1572 | CG | GLN | B | 100 | 33.742 | −16.434 | −34.842 | 1.00 | 36.31 | C |
| ATOM | 1573 | CD | GLN | B | 100 | 34.267 | −16.405 | −33.444 | 1.00 | 38.38 | C |
| ATOM | 1574 | OE1 | GLN | B | 100 | 33.505 | −16.427 | −32.483 | 1.00 | 39.66 | O |
| ATOM | 1575 | NE2 | GLN | B | 100 | 35.581 | −16.373 | −33.311 | 1.00 | 37.87 | N |
| ATOM | 1576 | C | GLN | B | 100 | 33.873 | −15.216 | −37.633 | 1.00 | 34.23 | C |
| ATOM | 1577 | O | GLN | B | 100 | 34.924 | −14.626 | −37.359 | 1.00 | 34.81 | O |
| ATOM | 1578 | N | LYS | B | 101 | 33.744 | −16.013 | −38.700 | 1.00 | 34.12 | N |
| ATOM | 1579 | CA | LYS | B | 101 | 34.860 | −16.292 | −39.593 | 1.00 | 34.67 | C |
| ATOM | 1580 | CB | LYS | B | 101 | 34.516 | −17.429 | −40.572 | 1.00 | 36.97 | C |
| ATOM | 1581 | CG | LYS | B | 101 | 35.632 | −17.838 | −41.542 | 1.00 | 38.53 | C |
| ATOM | 1582 | CD | LYS | B | 101 | 36.924 | −18.280 | −40.846 | 1.00 | 41.54 | C |
| ATOM | 1583 | CE | LYS | B | 101 | 38.050 | −18.546 | −41.822 | 1.00 | 42.78 | C |
| ATOM | 1584 | NZ | LYS | B | 101 | 37.826 | −19.788 | −42.607 | 1.00 | 42.83 | N |
| ATOM | 1585 | C | LYS | B | 101 | 35.229 | −15.030 | −40.357 | 1.00 | 32.62 | C |
| ATOM | 1586 | O | LYS | B | 101 | 34.366 | −14.402 | −40.964 | 1.00 | 29.98 | O |
| ATOM | 1587 | N | THR | B | 102 | 36.508 | −14.659 | −40.309 | 1.00 | 31.49 | N |
| ATOM | 1588 | CA | THR | B | 102 | 37.001 | −13.500 | −41.037 | 1.00 | 31.48 | C |
| ATOM | 1589 | CB | THR | B | 102 | 38.469 | −13.211 | −40.692 | 1.00 | 30.59 | C |
| ATOM | 1590 | OG1 | THR | B | 102 | 38.586 | −13.015 | −39.277 | 1.00 | 29.98 | O |
| ATOM | 1591 | CG2 | THR | B | 102 | 38.978 | −11.964 | −41.427 | 1.00 | 30.24 | C |
| ATOM | 1592 | C | THR | B | 102 | 36.844 | −13.705 | −42.544 | 1.00 | 31.36 | C |
| ATOM | 1593 | O | THR | B | 102 | 37.052 | −14.800 | −43.065 | 1.00 | 30.63 | O |
| ATOM | 1594 | N | TYR | B | 103 | 36.436 | −12.639 | −43.228 | 1.00 | 31.92 | N |
| ATOM | 1595 | CA | TYR | B | 103 | 36.154 | −12.680 | −44.649 | 1.00 | 33.48 | C |
| ATOM | 1596 | CB | TYR | B | 103 | 34.752 | −13.235 | −44.846 | 1.00 | 36.04 | C |
| ATOM | 1597 | CG | TYR | B | 103 | 34.206 | −13.232 | −46.255 | 1.00 | 37.46 | C |
| ATOM | 1598 | CD1 | TYR | B | 103 | 34.868 | −13.897 | −47.287 | 1.00 | 38.95 | C |
| ATOM | 1599 | CE1 | TYR | B | 103 | 34.359 | −13.909 | −48.584 | 1.00 | 40.44 | C |
| ATOM | 1600 | CZ | TYR | B | 103 | 33.203 | −13.194 | −48.873 | 1.00 | 40.62 | C |
| ATOM | 1601 | OH | TYR | B | 103 | 32.701 | −13.169 | −50.155 | 1.00 | 42.86 | O |
| ATOM | 1602 | CE2 | TYR | B | 103 | 32.548 | −12.490 | −47.871 | 1.00 | 40.69 | C |
| ATOM | 1603 | CD2 | TYR | B | 103 | 33.058 | −12.503 | −46.572 | 1.00 | 39.33 | C |
| ATOM | 1604 | C | TYR | B | 103 | 36.328 | −11.281 | −45.219 | 1.00 | 32.31 | C |
| ATOM | 1605 | O | TYR | B | 103 | 35.494 | −10.413 | −44.976 | 1.00 | 30.78 | O |
| ATOM | 1606 | N | GLN | B | 104 | 37.423 | −11.057 | −45.946 | 1.00 | 32.57 | N |
| ATOM | 1607 | CA | GLN | B | 104 | 37.687 | −9.740 | −46.538 | 1.00 | 33.53 | C |
| ATOM | 1608 | CB | GLN | B | 104 | 39.140 | −9.616 | −46.994 | 1.00 | 34.15 | C |
| ATOM | 1609 | CG | GLN | B | 104 | 40.152 | −9.729 | −45.860 | 1.00 | 34.64 | C |
| ATOM | 1610 | CD | GLN | B | 104 | 41.506 | −9.102 | −46.176 | 1.00 | 35.45 | C |
| ATOM | 1611 | OE1 | GLN | B | 104 | 41.790 | −8.711 | −47.315 | 1.00 | 33.99 | O |
| ATOM | 1612 | NE2 | GLN | B | 104 | 42.350 | −8.985 | −45.147 | 1.00 | 36.64 | N |
| ATOM | 1613 | C | GLN | B | 104 | 36.746 | −9.433 | −47.706 | 1.00 | 33.99 | C |
| ATOM | 1614 | O | GLN | B | 104 | 36.426 | −8.269 | −47.952 | 1.00 | 33.12 | O |
| ATOM | 1615 | N | GLY | B | 105 | 36.322 | −10.473 | −48.427 | 1.00 | 35.16 | N |
| ATOM | 1616 | CA | GLY | B | 105 | 35.349 | −10.331 | −49.506 | 1.00 | 36.65 | C |
| ATOM | 1617 | C | GLY | B | 105 | 35.903 | −9.649 | −50.742 | 1.00 | 38.59 | C |
| ATOM | 1618 | O | GLY | B | 105 | 37.112 | −9.388 | −50.850 | 1.00 | 41.05 | O |
| ATOM | 1619 | N | SER | B | 106 | 34.997 | −9.338 | −51.670 | 1.00 | 38.39 | N |
| ATOM | 1620 | CA | SER | B | 106 | 35.339 | −8.698 | −52.949 | 1.00 | 38.89 | C |
| ATOM | 1621 | CB | SER | B | 106 | 34.080 | −8.514 | −53.798 | 1.00 | 38.65 | C |
| ATOM | 1622 | OG | SER | B | 106 | 33.428 | −9.754 | −53.964 | 1.00 | 38.18 | O |
| ATOM | 1623 | C | SER | B | 106 | 35.991 | −7.332 | −52.793 | 1.00 | 39.96 | C |
| ATOM | 1624 | O | SER | B | 106 | 36.886 | −6.974 | −53.563 | 1.00 | 40.79 | O |
| ATOM | 1625 | N | TYR | B | 107 | 35.528 | −6.578 | −51.797 | 1.00 | 38.63 | N |
| ATOM | 1626 | CA | TYR | B | 107 | 35.950 | −5.201 | −50.586 | 1.00 | 36.54 | C |
| ATOM | 1627 | CB | TYR | B | 107 | 34.762 | −4.391 | −51.057 | 1.00 | 38.85 | C |
| ATOM | 1628 | CG | TYR | B | 107 | 33.583 | −4.441 | −52.005 | 1.00 | 40.94 | C |
| ATOM | 1629 | CD1 | TYR | B | 107 | 33.545 | −3.642 | −53.143 | 1.00 | 42.58 | C |
| ATOM | 1630 | CE1 | TYR | B | 107 | 32.477 | −3.701 | −54.033 | 1.00 | 43.48 | C |
| ATOM | 1631 | CZ | TYR | B | 107 | 31.433 | −4.575 | −33.788 | 1.00 | 42.88 | C |
| ATOM | 1632 | OH | TYR | B | 107 | 30.378 | −4.625 | −54.668 | 1.00 | 45.34 | O |
| ATOM | 1633 | CE2 | TYR | B | 107 | 31.458 | −5.394 | −52.672 | 1.00 | 43.18 | C |
| ATOM | 1634 | CD2 | TYR | B | 107 | 32.532 | −5.331 | −51.793 | 1.00 | 41.85 | C |
| ATOM | 1635 | C | TYR | B | 107 | 37.178 | −5.044 | −50.677 | 1.00 | 32.97 | C |
| ATOM | 1636 | O | TYR | B | 107 | 37.642 | −3.928 | −50.460 | 1.00 | 31.44 | O |
| ATOM | 1637 | N | GLY | B | 108 | 37.734 | −6.144 | −50.185 | 1.00 | 30.72 | N |
| ATOM | 1638 | CA | GLY | B | 108 | 38.934 | −6.101 | 49.338 | 1.00 | 30.85 | C |
| ATOM | 1639 | C | GLY | B | 108 | 38.678 | −5.427 | −47.998 | 1.00 | 29.42 | C |
| ATOM | 1640 | O | GLY | B | 108 | 39.413 | −4.524 | −47.609 | 1.00 | 28.81 | O |
| ATOM | 1641 | N | PHE | B | 109 | 37.622 | −5.865 | −47.316 | 1.00 | 27.69 | N |
| ATOM | 1642 | CA | PHE | B | 109 | 37.173 | −5.271 | −46.050 | 1.00 | 27.26 | C |
| ATOM | 1643 | CB | PHE | B | 109 | 35.713 | −5.669 | −45.783 | 1.00 | 27.38 | C |
| ATOM | 1644 | CG | PHE | B | 109 | 35.177 | −5.205 | −44.456 | 1.00 | 26.86 | C |
| ATOM | 1645 | CD1 | PHE | B | 109 | 35.185 | −3.846 | −44.118 | 1.00 | 26.92 | C |
| ATOM | 1646 | CE1 | PHE | B | 109 | 34.686 | −3.418 | −42.895 | 1.00 | 26.69 | C |
| ATOM | 1647 | CZ | PHE | B | 109 | 34.149 | −4.338 | −42.011 | 1.00 | 25.73 | C |
| ATOM | 1648 | CE2 | PHE | B | 109 | 34.103 | −5.681 | −42.342 | 1.00 | 25.59 | C |
| ATOM | 1649 | CD2 | PHE | B | 109 | 34.620 | −6.114 | −43.561 | 1.00 | 26.53 | C |
| ATOM | 1650 | C | PHE | B | 109 | 38.059 | −5.714 | −44.884 | 1.00 | 26.48 | C |
| ATOM | 1651 | O | PHE | B | 109 | 38.201 | −6.915 | −44.636 | 1.00 | 25.41 | O |
| ATOM | 1652 | N | ARG | B | 110 | 38.644 | −4.742 | −44.182 | 1.00 | 25.82 | N |
| ATOM | 1653 | CA | ARG | B | 110 | 39.469 | −4.993 | −42.993 | 1.00 | 26.65 | C |
| ATOM | 1654 | CB | ARG | B | 110 | 40.968 | −4.939 | −43.338 | 1.00 | 28.83 | C |
| ATOM | 1655 | CG | ARG | B | 110 | 41.381 | −5.657 | −44.621 | 1.00 | 31.03 | C |
| ATOM | 1656 | CD | ARG | B | 110 | 42.845 | −5.548 | −44.975 | 1.00 | 32.79 | C |
| ATOM | 1657 | NE | ARG | B | 110 | 43.383 | −4.188 | −44.977 | 1.00 | 35.48 | N |
| ATOM | 1658 | CZ | ARG | B | 110 | 43.089 | −3.226 | −45.860 | 1.00 | 35.96 | C |
| ATOM | 1659 | NH1 | ARG | B | 110 | 42.213 | −3.400 | −46.841 | 1.00 | 36.76 | N |
| ATOM | 1660 | NH2 | ARG | B | 110 | 43.683 | −2.043 | −45.732 | 1.00 | 35.64 | N |
| ATOM | 1661 | C | ARG | B | 110 | 39.155 | −3.918 | −41.940 | 1.00 | 24.99 | C |
| ATOM | 1662 | O | ARG | B | 110 | 38.637 | −2.846 | −42.276 | 1.00 | 25.90 | O |
| ATOM | 1663 | N | LEU | B | 111 | 39.472 | −4.205 | −40.679 | 1.00 | 23.80 | N |
| ATOM | 1664 | CA | LEU | B | 111 | 39.308 | −3.245 | −39.585 | 1.00 | 23.14 | C |
| ATOM | 1665 | CB | LEU | B | 111 | 38.716 | −3.914 | −38.346 | 1.00 | 22.78 | C |
| ATOM | 1666 | CG | LEU | B | 111 | 37.315 | −4.489 | −38.478 | 1.00 | 22.83 | C |
| ATOM | 1667 | CD1 | LEU | B | 111 | 36.953 | −5.211 | −37.196 | 1.00 | 23.30 | C |
| ATOM | 1668 | CD2 | LEU | B | 111 | 36.291 | −3.415 | −38.792 | 1.00 | 23.00 | C |
| ATOM | 1669 | C | LEU | B | 111 | 40.637 | −2.601 | −39.204 | 1.00 | 22.66 | C |
| ATOM | 1670 | O | LEU | B | 111 | 41.692 | −3.213 | −39.341 | 1.00 | 21.46 | O |
| ATOM | 1671 | N | GLY | B | 112 | 40.555 | −1.359 | −38.733 | 1.00 | 21.86 | N |
| ATOM | 1672 | CA | GLY | B | 112 | 41.694 | −0.614 | −38.200 | 1.00 | 21.25 | C |
| ATOM | 1673 | C | GLY | B | 112 | 41.328 | 0.028 | −36.882 | 1.00 | 20.91 | C |
| ATOM | 1674 | O | GLY | B | 112 | 40.149 | 0.210 | −36.579 | 1.00 | 20.68 | O |
| ATOM | 1675 | N | PHE | B | 113 | 42.336 | 0.354 | −36.086 | 1.00 | 20.98 | N |
| ATOM | 1676 | CA | PHE | B | 113 | 42.154 | 0.932 | −34.756 | 1.00 | 20.67 | C |
| ATOM | 1677 | CB | PHE | B | 113 | 42.296 | −0.160 | −33.695 | 1.00 | 20.94 | C |
| ATOM | 1678 | CG | PHE | B | 113 | 41.437 | −1.363 | −33.962 | 1.00 | 21.81 | C |
| ATOM | 1679 | CD1 | PHE | B | 113 | 40.105 | −1.386 | −33.563 | 1.00 | 22.26 | C |
| ATOM | 1680 | CE1 | PHE | B | 113 | 39.298 | −2.492 | −33.826 | 1.00 | 22.77 | C |
| ATOM | 1681 | CZ | PHE | B | 113 | 39.819 | −3.571 | −34.512 | 1.00 | 22.37 | C |
| ATOM | 1682 | CE2 | PHE | B | 113 | 41.139 | −3.553 | −34.929 | 1.00 | 22.28 | C |
| ATOM | 1683 | CD2 | PHE | B | 113 | 41.939 | −2.454 | −34.655 | 1.00 | 21.66 | C |
| ATOM | 1684 | C | PHE | B | 113 | 43.183 | 2.029 | −34.547 | 1.00 | 20.39 | C |
| ATOM | 1685 | O | PHE | B | 113 | 44.238 | 1.998 | −35.159 | 1.00 | 19.81 | O |
| ATOM | 1686 | N | LEU | B | 114 | 42.869 | 3.012 | −33.712 | 1.00 | 20.19 | N |
| ATOM | 1687 | CA | LEU | B | 114 | 43.846 | 4.042 | −33.363 | 1.00 | 20.20 | C |
| ATOM | 1688 | CB | LEU | B | 114 | 43.211 | 5.192 | −32.570 | 1.00 | 20.43 | C |
| ATOM | 1689 | CG | LEU | B | 114 | 42.216 | 6.089 | −33.331 | 1.00 | 20.53 | C |
| ATOM | 1690 | CD1 | LEU | B | 114 | 41.703 | 7.178 | −32.400 | 1.00 | 20.57 | C |
| ATOM | 1691 | CD2 | LEU | B | 114 | 42.795 | 6.700 | −34.614 | 1.00 | 20.52 | C |
| ATOM | 1692 | C | LEU | B | 114 | 44.990 | 3.419 | −32.562 | 1.00 | 20.11 | C |
| ATOM | 1693 | O | LEU | B | 114 | 44.782 | 2.436 | −31.859 | 1.00 | 19.26 | O |
| ATOM | 1694 | N | HIS | B | 115 | 46.181 | 3.994 | −32.706 | 1.00 | 19.54 | N |
| ATOM | 1695 | CA | HIS | B | 115 | 47.379 | 3.530 | −32.016 | 1.00 | 19.90 | C |
| ATOM | 1696 | CB | HIS | B | 115 | 48.579 | 3.597 | −32.960 | 1.00 | 19.48 | C |
| ATOM | 1697 | CG | HIS | B | 115 | 48.339 | 2.923 | −34.275 | 1.00 | 20.03 | C |
| ATOM | 1698 | ND1 | HIS | B | 115 | 48.196 | 3.619 | −35.460 | 1.00 | 19.66 | N |
| ATOM | 1699 | CE1 | HIS | B | 115 | 47.939 | 2.768 | −36.437 | 1.00 | 19.79 | C |
| ATOM | 1700 | NE2 | HIS | B | 115 | 47.951 | 1.543 | −35.940 | 1.00 | 20.01 | N |
| ATOM | 1701 | CD2 | HIS | B | 115 | 48.174 | 1.614 | −34.586 | 1.00 | 20.29 | C |
| ATOM | 1702 | C | HIS | B | 115 | 47.574 | 4.385 | −30.753 | 1.00 | 20.10 | C |
| ATOM | 1703 | O | HIS | B | 115 | 48.378 | 5.321 | −30.722 | 1.00 | 19.64 | O |
| ATOM | 1704 | N | SER | B | 116 | 46.794 | 4.061 | −29.725 | 1.00 | 19.94 | N |
| ATOM | 1705 | CA | SER | B | 116 | 46.549 | 4.977 | −28.616 | 1.00 | 20.39 | C |
| ATOM | 1706 | CB | SER | B | 116 | 45.094 | 4.850 | −28.169 | 1.00 | 20.71 | C |
| ATOM | 1707 | OG | SER | B | 116 | 44.227 | 5.141 | −29.253 | 1.00 | 20.10 | O |
| ATOM | 1708 | C | SER | B | 116 | 47.483 | 4.825 | −27.421 | 1.00 | 20.27 | C |
| ATOM | 1709 | O | SER | B | 116 | 47.545 | 5.736 | −26.600 | 1.00 | 19.30 | O |
| ATOM | 1710 | N | GLY | B | 117 | 48.164 | 3.684 | −27.323 | 1.00 | 20.10 | N |
| ATOM | 1711 | CA | GLY | B | 117 | 49.123 | 3.443 | −26.235 | 1.00 | 19.98 | C |
| ATOM | 1712 | C | GLY | B | 117 | 48.432 | 3.052 | −24.949 | 1.00 | 20.37 | C |
| ATOM | 1713 | O | GLY | B | 117 | 47.214 | 2.890 | −24.924 | 1.00 | 20.85 | O |
| ATOM | 1714 | N | THR | B | 118 | 49.200 | 2.903 | −23.876 | 1.00 | 21.36 | N |
| ATOM | 1715 | CA | THR | B | 118 | 48.663 | 2.404 | −22.596 | 1.00 | 22.28 | C |
| ATOM | 1716 | CB | THR | B | 118 | 49.239 | 1.015 | −22.247 | 1.00 | 21.52 | C |
| ATOM | 1717 | OG1 | THR | B | 118 | 50.666 | 1.072 | −22.181 | 1.00 | 22.06 | O |
| ATOM | 1718 | CG2 | THR | B | 118 | 48.836 | 0.008 | −23.286 | 1.00 | 21.59 | C |
| ATOM | 1719 | C | THR | B | 118 | 48.863 | 3.365 | −21.423 | 1.00 | 23.54 | C |
| ATOM | 1720 | O | THR | B | 118 | 48.867 | 2.934 | −20.267 | 1.00 | 25.30 | O |
| ATOM | 1721 | N | ALA | B | 119 | 48.984 | 4.664 | −21.715 | 1.00 | 24.23 | N |
| ATOM | 1722 | CA | ALA | B | 119 | 49.034 | 5.686 | −20.681 | 1.00 | 26.55 | C |
| ATOM | 1723 | CB | ALA | B | 119 | 49.227 | 7.067 | −21.310 | 1.00 | 26.18 | C |
| ATOM | 1724 | C | ALA | B | 119 | 47.757 | 5.662 | −19.831 | 1.00 | 29.21 | C |
| ATOM | 1725 | O | ALA | B | 119 | 46.659 | 5.389 | −20.342 | 1.00 | 29.57 | O |
| ATOM | 1726 | N | LYS | B | 120 | 47.921 | 5.962 | −18.544 | 1.00 | 31.32 | N |
| ATOM | 1727 | CA | LYS | B | 120 | 46.829 | 5.897 | −17.550 | 1.00 | 33.08 | C |
| ATOM | 1728 | CB | LYS | B | 120 | 47.325 | 6.355 | −16.166 | 1.00 | 35.87 | C |
| ATOM | 1729 | CG | LYS | B | 120 | 46.823 | 5.518 | −14.969 | 1.00 | 37.85 | C |
| ATOM | 1730 | CD | LYS | B | 120 | 47.773 | 5.575 | −13.768 | 1.00 | 40.03 | C |
| ATOM | 1731 | CE | LYS | B | 120 | 49.028 | 4.706 | −13.936 | 1.00 | 40.75 | C |
| ATOM | 1732 | NZ | LYS | B | 120 | 49.578 | 4.203 | −12.640 | 1.00 | 41.01 | N |
| ATOM | 1733 | C | LYS | B | 120 | 45.582 | 6.701 | −17.958 | 1.00 | 32.69 | C |
| ATOM | 1734 | O | LYS | B | 120 | 44.462 | 6.291 | −17.663 | 1.00 | 31.85 | O |
| ATOM | 1735 | N | SER | B | 121 | 45.785 | 7.819 | −18.651 | 1.00 | 33.17 | N |
| ATOM | 1736 | CA | SER | B | 121 | 44.698 | 8.687 | −19.112 | 1.00 | 33.79 | C |
| ATOM | 1737 | CB | SER | B | 121 | 45.273 | 10.078 | −19.425 | 1.00 | 34.57 | C |
| ATOM | 1738 | OG | SER | B | 121 | 46.118 | 10.027 | −20.566 | 1.00 | 35.73 | O |
| ATOM | 1739 | C | SER | B | 121 | 43.908 | 8.202 | −20.351 | 1.00 | 33.34 | C |
| ATOM | 1740 | O | SER | B | 121 | 43.000 | 8.908 | −20.806 | 1.00 | 32.74 | O |
| ATOM | 1741 | N | VAL | B | 122 | 44.248 | 7.035 | −20.903 | 1.00 | 32.07 | N |
| ATOM | 1742 | CA | VAL | B | 122 | 43.640 | 6.577 | −22.155 | 1.00 | 31.29 | C |
| ATOM | 1743 | CB | VAL | B | 122 | 44.578 | 5.598 | −22.917 | 1.00 | 31.26 | C |
| ATOM | 1744 | CG1 | VAL | B | 122 | 44.526 | 4.177 | −22.363 | 1.00 | 30.97 | C |
| ATOM | 1745 | CG2 | VAL | B | 122 | 44.270 | 5.607 | −24.399 | 1.00 | 32.83 | C |
| ATOM | 1746 | C | VAL | B | 122 | 42.241 | 5.996 | −21.873 | 1.00 | 30.73 | C |
| ATOM | 1747 | O | VAL | B | 122 | 42.067 | 5.176 | −20.971 | 1.00 | 30.12 | O |
| ATOM | 1748 | N | THR | B | 123 | 41.246 | 6.461 | −22.627 | 1.00 | 29.53 | N |
| ATOM | 1749 | CA | THR | B | 123 | 39.859 | 6.002 | −22.474 | 1.00 | 29.12 | C |
| ATOM | 1750 | CB | THR | B | 123 | 38.858 | 7.117 | −22.810 | 1.00 | 29.07 | C |
| ATOM | 1751 | OG1 | THR | B | 123 | 39.088 | 7.579 | −24.142 | 1.00 | 29.42 | O |
| ATOM | 1752 | CG2 | THR | B | 123 | 39.006 | 8.273 | −21.835 | 1.00 | 29.74 | C |
| ATOM | 1753 | C | THR | B | 123 | 39.546 | 4.785 | −23.344 | 1.00 | 29.22 | C |
| ATOM | 1754 | O | THR | B | 123 | 38.635 | 4.021 | −23.030 | 1.00 | 27.86 | O |
| ATOM | 1755 | N | CYS | B | 124 | 40.297 | 4.623 | −24.434 | 1.00 | 28.09 | N |
| ATOM | 1756 | CA | CYS | B | 124 | 40.125 | 3.509 | −25.366 | 1.00 | 27.92 | C |
| ATOM | 1757 | CB | CYS | B | 124 | 39.142 | 3.941 | −26.456 | 1.00 | 29.34 | C |
| ATOM | 1758 | SG | CYS | B | 124 | 38.736 | 2.650 | −27.650 | 1.00 | 31.61 | S |
| ATOM | 1759 | C | CYS | B | 124 | 41.474 | 3.127 | −25.993 | 1.00 | 26.21 | C |
| ATOM | 1760 | O | CYS | B | 124 | 42.195 | 4.000 | −26.465 | 1.00 | 26.94 | O |
| ATOM | 1761 | N | THR | B | 125 | 41.819 | 1.841 | −25.978 | 1.00 | 23.59 | N |
| ATOM | 1762 | CA | THR | B | 125 | 43.064 | 1.359 | −26.590 | 1.00 | 21.82 | C |
| ATOM | 1763 | CB | THR | B | 125 | 44.282 | 1.498 | −25.646 | 1.00 | 21.34 | C |
| ATOM | 1764 | OG1 | THR | B | 125 | 45.484 | 1.086 | −26.322 | 1.00 | 19.38 | O |
| ATOM | 1765 | CG2 | THR | B | 125 | 44.102 | 0.662 | −24.355 | 1.00 | 21.66 | C |
| ATOM | 1766 | C | THR | B | 125 | 42.946 | −0.088 | −27.078 | 1.00 | 21.96 | C |
| ATOM | 1767 | O | THR | B | 125 | 42.304 | −0.925 | −26.432 | 1.00 | 20.98 | O |
| ATOM | 1768 | N | TYR | B | 126 | 43.582 | −0.357 | −28.220 | 1.00 | 21.33 | N |
| ATOM | 1769 | CA | TYR | B | 126 | 43.524 | −1.651 | −28.890 | 1.00 | 21.11 | C |
| ATOM | 1770 | CB | TYR | B | 126 | 43.124 | −1.438 | −30.347 | 1.00 | 22.44 | C |
| ATOM | 1771 | CG | TYR | B | 126 | 43.181 | −2.678 | −31.201 | 1.00 | 23.41 | C |
| ATOM | 1772 | CD1 | TYR | B | 126 | 42.200 | −3.664 | −31.100 | 1.00 | 24.42 | C |
| ATOM | 1773 | CE1 | TYR | B | 126 | 42.248 | −4.809 | −31.891 | 1.00 | 25.06 | C |
| ATOM | 1774 | CZ | TYR | B | 126 | 43.285 | −4.968 | −32.790 | 1.00 | 24.63 | C |
| ATOM | 1775 | OH | TYR | B | 126 | 43.350 | −6.077 | −33.588 | 1.00 | 26.01 | O |
| ATOM | 1776 | CE2 | TYR | B | 126 | 44.267 | −4.005 | −32.908 | 1.00 | 24.68 | C |
| ATOM | 1777 | CD2 | TYR | B | 126 | 44.211 | −2.867 | −32.115 | 1.00 | 23.98 | C |
| ATOM | 1778 | C | TYR | B | 126 | 44.873 | −2.352 | −28.823 | 1.00 | 20.10 | C |
| ATOM | 1779 | O | TYR | B | 126 | 45.900 | −1.737 | −29.064 | 1.00 | 20.10 | O |
| ATOM | 1780 | N | SER | B | 127 | 44.846 | −3.642 | −28.503 | 1.00 | 19.53 | N |
| ATOM | 1781 | CA | SER | B | 127 | 46.033 | −4.498 | −28.464 | 1.00 | 19.14 | C |
| ATOM | 1782 | CB | SER | B | 127 | 45.947 | −5.451 | −27.273 | 1.00 | 18.94 | C |
| ATOM | 1783 | OG | SER | B | 127 | 46.908 | −6.487 | −27.393 | 1.00 | 18.11 | O |
| ATOM | 1784 | C | SER | B | 127 | 46.091 | −5.322 | −29.746 | 1.00 | 19.02 | C |
| ATOM | 1785 | O | SER | B | 127 | 45.249 | −6.194 | −29.928 | 1.00 | 18.80 | O |
| ATOM | 1786 | N | PRO | B | 128 | 47.067 | −5.047 | −30.637 | 1.00 | 19.12 | N |
| ATOM | 1787 | CA | PRO | B | 128 | 47.230 | −5.906 | −31.816 | 1.00 | 19.18 | C |
| ATOM | 1788 | CB | PRO | B | 128 | 48.378 | −5.244 | −32.585 | 1.00 | 19.44 | C |
| ATOM | 1789 | CG | PRO | B | 128 | 48.331 | −3.811 | −32.180 | 1.00 | 19.07 | C |
| ATOM | 1790 | CD | PRO | B | 128 | 47.846 | −3.795 | −30.758 | 1.00 | 19.00 | C |
| ATOM | 1791 | C | PRO | B | 128 | 47.571 | −7.359 | −31.492 | 1.00 | 19.40 | C |
| ATOM | 1792 | O | PRO | B | 128 | 47.083 | −8.259 | −32.169 | 1.00 | 19.84 | O |
| ATOM | 1793 | N | ALA | B | 129 | 48.380 | −7.589 | −30.465 | 1.00 | 19.72 | N |
| ATOM | 1794 | CA | ALA | B | 129 | 48.806 | −8.948 | −30.111 | 1.00 | 20.15 | C |
| ATOM | 1795 | CB | ALA | B | 129 | 49.867 | −8.908 | −29.027 | 1.00 | 20.26 | C |
| ATOM | 1796 | C | ALA | C | 129 | 47.637 | −9.825 | −29.673 | 1.00 | 21.09 | C |
| ATOM | 1797 | O | ALA | B | 129 | 47.589 | −11.009 | −30.017 | 1.00 | 21.84 | O |
| ATOM | 1798 | N | LEU | B | 130 | 46.694 | −9.234 | −28.948 | 1.00 | 21.55 | N |
| ATOM | 1799 | CA | LEU | B | 130 | 45.520 | −9.946 | −28.444 | 1.00 | 22.17 | C |
| ATOM | 1800 | CB | LEU | B | 130 | 45.198 | −9.475 | −27.021 | 1.00 | 21.91 | C |
| ATOM | 1801 | CG | LEU | B | 130 | 46.204 | −9.793 | −25.920 | 1.00 | 22.02 | C |
| ATOM | 1802 | CD1 | LEU | B | 130 | 45.902 | −8.915 | −24.692 | 1.00 | 21.60 | C |
| ATOM | 1803 | CD2 | LEU | B | 130 | 46.240 | −11.267 | −25.538 | 1.00 | 22.22 | C |
| ATOM | 1804 | C | LEU | B | 130 | 44.265 | −9.770 | −29.322 | 1.00 | 22.76 | C |
| ATOM | 1805 | O | LEU | B | 130 | 43.263 | −10.450 | −29.088 | 1.00 | 22.97 | O |
| ATOM | 1806 | N | ASN | B | 131 | 44.315 | −8.874 | −30.318 | 1.00 | 22.81 | N |
| ATOM | 1807 | CA | ASN | B | 131 | 43.132 | −8.460 | −31.093 | 1.00 | 22.52 | C |
| ATOM | 1808 | CB | ASN | B | 131 | 42.737 | −9.538 | −32.119 | 1.00 | 22.00 | C |
| ATOM | 1809 | CG | ASN | B | 131 | 41.652 | −9.074 | −33.085 | 1.00 | 21.56 | C |
| ATOM | 1810 | OD1 | ASN | B | 131 | 41.533 | −7.884 | −33.396 | 1.00 | 21.11 | O |
| ATOM | 1811 | ND2 | ASN | B | 131 | 40.842 | −10.015 | −33.548 | 1.00 | 20.58 | N |
| ATOM | 1812 | C | ASN | B | 131 | 41.979 | −8.117 | −30.143 | 1.00 | 23.19 | C |
| ATOM | 1813 | O | ASN | B | 131 | 40.859 | −8.636 | −30.259 | 1.00 | 22.66 | O |
| ATOM | 1814 | N | LYS | B | 132 | 42.291 | −7.247 | −29.189 | 1.00 | 24.34 | N |
| ATOM | 1815 | CA | LYS | B | 132 | 41.404 | −6.947 | −28.078 | 1.00 | 25.20 | C |
| ATOM | 1816 | CB | LYS | B | 132 | 41.883 | −7.678 | −26.826 | 1.00 | 24.92 | C |
| ATOM | 1817 | CG | LYS | B | 132 | 40.894 | −7.625 | −25.671 | 1.00 | 24.94 | C |
| ATOM | 1818 | CD | LYS | B | 132 | 41.277 | −8.597 | −24.601 | 1.00 | 25.45 | C |
| ATOM | 1819 | CE | LYS | B | 132 | 40.363 | −8.478 | −23.389 | 1.00 | 25.83 | C |
| ATOM | 1820 | NZ | LYS | B | 132 | 40.609 | −9.568 | −22.392 | 1.00 | 26.31 | N |
| ATOM | 1821 | C | LYS | B | 132 | 41.329 | −5.445 | −27.814 | 1.00 | 24.92 | C |
| ATOM | 1822 | O | LYS | B | 132 | 42.345 | −4.786 | −27.645 | 1.00 | 24.39 | O |
| ATOM | 1823 | N | MET | B | 133 | 40.115 | −4.928 | −27.779 | 1.00 | 26.64 | N |
| ATOM | 1824 | CA | MET | B | 133 | 39.853 | −3.534 | −27.463 | 1.00 | 27.82 | C |
| ATOM | 1825 | CB | MET | B | 133 | 38.636 | −3.061 | −28.252 | 1.00 | 29.01 | C |
| ATOM | 1826 | CG | MET | B | 133 | 38.270 | −1.612 | −28.031 | 1.00 | 32.37 | C |
| ATOM | 1827 | SD | MET | B | 133 | 39.448 | −0.433 | −28.730 | 1.00 | 37.04 | S |
| ATOM | 1828 | CE | MET | B | 133 | 38.956 | −0.546 | −30.435 | 1.00 | 36.37 | C |
| ATOM | 1829 | C | MET | B | 133 | 39.601 | −3.415 | −25.958 | 1.00 | 28.42 | C |
| ATOM | 1830 | O | MET | B | 133 | 38.870 | −4.218 | −25.376 | 1.00 | 28.59 | O |
| ATOM | 1831 | N | PHE | B | 134 | 40.231 | −2.424 | −25.332 | 1.00 | 28.12 | N |
| ATOM | 1832 | CA | PHE | B | 134 | 39.987 | −2.078 | −23.934 | 1.00 | 28.36 | C |
| ATOM | 1833 | CB | PHE | B | 134 | 41.292 | −2.096 | −23.137 | 1.00 | 28.24 | C |
| ATOM | 1834 | CG | PHE | B | 134 | 41.961 | −3.448 | −23.091 | 1.00 | 28.37 | C |
| ATOM | 1835 | CD1 | PHE | B | 134 | 42.710 | −3.911 | −24.170 | 1.00 | 27.47 | C |
| ATOM | 1836 | CE1 | PHE | B | 134 | 43.318 | −5.158 | −24.131 | 1.00 | 28.35 | C |
| ATOM | 1837 | CZ | PHE | B | 134 | 43.191 | −5.959 | −23.003 | 1.00 | 28.47 | C |
| ATOM | 1838 | CE2 | PHE | B | 134 | 42.441 | −5.502 | −21.927 | 1.00 | 28.53 | C |
| ATOM | 1839 | CD2 | PHE | B | 134 | 41.835 | −4.262 | −21.973 | 1.00 | 28.02 | C |
| ATOM | 1840 | C | PHE | B | 134 | 39.385 | −0.679 | −23.913 | 1.00 | 29.30 | C |
| ATOM | 1841 | O | PHE | B | 134 | 40.007 | 0.252 | −24.425 | 1.00 | 28.06 | O |
| ATOM | 1842 | N | CYS | B | 135 | 38.173 | −0.535 | −23.362 | 1.00 | 30.67 | N |
| ATOM | 1843 | CA | CYS | B | 135 | 37.513 | 0.770 | −23.273 | 1.00 | 31.96 | C |
| ATOM | 1844 | CB | CYS | B | 135 | 36.478 | 0.974 | −24.383 | 1.00 | 32.65 | C |
| ATOM | 1845 | SG | CYS | B | 135 | 35.322 | −0.357 | −24.523 | 1.00 | 34.97 | S |
| ATOM | 1846 | C | CYS | B | 135 | 36.829 | 1.003 | −21.933 | 1.00 | 31.33 | C |
| ATOM | 1847 | O | CYS | B | 135 | 36.493 | 0.062 | −21.216 | 1.00 | 30.55 | O |
| ATOM | 1848 | N | GLN | B | 136 | 36.613 | 2.279 | −21.630 | 1.00 | 31.71 | N |
| ATOM | 1849 | CA | GLN | B | 136 | 35.802 | 2.689 | −20.499 | 1.00 | 32.43 | C |
| ATOM | 1850 | CB | GLN | B | 136 | 36.234 | 4.067 | −19.997 | 1.00 | 33.28 | C |
| ATOM | 1851 | CG | GLN | B | 136 | 37.520 | 4.040 | −19.170 | 1.00 | 35.05 | C |
| ATOM | 1852 | CD | GLN | B | 136 | 37.871 | 5.414 | −18.603 | 1.00 | 36.87 | C |
| ATOM | 1853 | OE1 | GLN | B | 136 | 37.621 | 6.433 | −19.241 | 1.00 | 37.83 | O |
| ATOM | 1854 | NE2 | GLN | B | 136 | 38.458 | 5.442 | −17.391 | 1.00 | 38.51 | N |
| ATOM | 1855 | C | GLN | B | 136 | 34.321 | 2.670 | −20.876 | 1.00 | 32.53 | C |
| ATOM | 1856 | O | GLN | B | 136 | 33.964 | 2.865 | −22.050 | 1.00 | 29.77 | O |
| ATOM | 1857 | N | LEU | B | 137 | 33.474 | 2.413 | −19.877 | 1.00 | 33.23 | N |
| ATOM | 1858 | CA | LEU | B | 137 | 32.033 | 2.312 | −20.075 | 1.00 | 33.47 | C |
| ATOM | 1859 | CB | LEU | B | 137 | 32.317 | 1.912 | −18.773 | 1.00 | 35.18 | C |
| ATOM | 1860 | CG | LEU | B | 137 | 29.784 | 1.717 | −18.917 | 1.00 | 35.76 | C |
| ATOM | 1861 | CD1 | LEU | B | 137 | 29.244 | 0.362 | −18.455 | 1.00 | 35.48 | C |
| ATOM | 1862 | CD2 | LEU | B | 137 | 29.036 | 2.893 | −18.288 | 1.00 | 35.93 | C |
| ATOM | 1863 | C | LEU | B | 137 | 31.442 | 3.602 | −20.646 | 1.00 | 32.36 | C |
| ATOM | 1864 | O | LEU | B | 137 | 31.719 | 4.680 | −20.149 | 1.00 | 33.47 | O |
| ATOM | 1865 | N | ALA | B | 138 | 30.658 | 3.457 | −21.720 | 1.00 | 32.25 | N |
| ATOM | 1866 | CA | ALA | B | 138 | 29.972 | 4.569 | −22.386 | 1.00 | 32.36 | C |
| ATOM | 1867 | CB | ALA | B | 138 | 28.961 | 5.232 | −21.432 | 1.00 | 33.09 | C |
| ATOM | 1868 | C | ALA | B | 138 | 30.890 | 5.644 | −22.999 | 1.00 | 31.67 | C |
| ATOM | 1869 | O | ALA | B | 138 | 30.437 | 6.756 | −23.252 | 1.00 | 32.94 | O |
| ATOM | 1870 | N | LYS | B | 139 | 32.155 | 5.323 | −23.255 | 1.00 | 30.53 | N |
| ATOM | 1871 | CA | LYS | B | 139 | 33.083 | 6.251 | −23.889 | 1.00 | 30.88 | C |
| ATOM | 1872 | CB | LYS | B | 139 | 34.451 | 6.211 | −23.197 | 1.00 | 32.15 | C |
| ATOM | 1873 | CG | LYS | B | 139 | 34.425 | 6.617 | −21.724 | 1.00 | 33.34 | C |
| ATOM | 1874 | CD | LYS | B | 139 | 34.321 | 8.129 | −21.582 | 1.00 | 33.51 | C |
| ATOM | 1875 | CE | LYS | B | 139 | 33.846 | 8.556 | −20.202 | 1.00 | 35.39 | C |
| ATOM | 1876 | NZ | LYS | B | 139 | 33.532 | 10.010 | −20.225 | 1.00 | 36.41 | N |
| ATOM | 1877 | C | LYS | B | 139 | 33.257 | 5.909 | −25.354 | 1.00 | 29.73 | C |
| ATOM | 1878 | O | LYS | B | 139 | 33.050 | 4.762 | −25.768 | 1.00 | 26.89 | O |
| ATOM | 1879 | N | THR | B | 140 | 33.638 | 6.925 | −26.129 | 1.00 | 29.48 | N |
| ATOM | 1880 | CA | THR | B | 140 | 33.923 | 6.780 | −27.552 | 1.00 | 29.78 | C |
| ATOM | 1881 | CB | THR | B | 140 | 34.476 | 8.093 | −28.133 | 1.00 | 30.30 | C |
| ATOM | 1882 | OG1 | THR | B | 140 | 33.605 | 9.168 | −27.785 | 1.00 | 31.53 | O |
| ATOM | 1883 | CG2 | THR | B | 140 | 34.606 | 8.017 | −29.650 | 1.00 | 31.01 | C |
| ATOM | 1884 | C | THR | B | 140 | 34.939 | 5.674 | −27.809 | 1.00 | 29.65 | C |
| ATOM | 1885 | O | THR | B | 140 | 35.991 | 5.621 | −27.172 | 1.00 | 30.29 | O |
| ATOM | 1886 | N | CYS | B | 141 | 34.601 | 4.794 | −28.745 | 1.00 | 30.39 | N |
| ATOM | 1887 | CA | CYS | B | 141 | 35.454 | 3.684 | −29.132 | 1.00 | 31.47 | C |
| ATOM | 1888 | CB | CYS | B | 141 | 34.888 | 2.382 | −28.562 | 1.00 | 33.54 | C |
| ATOM | 1889 | SG | CYS | B | 141 | 35.996 | 0.975 | −28.653 | 1.00 | 43.16 | S |
| ATOM | 1890 | C | CYS | B | 141 | 35.508 | 3.684 | −30.660 | 1.00 | 30.43 | C |
| ATOM | 1891 | O | CYS | B | 141 | 34.601 | 3.151 | −31.310 | 1.00 | 31.53 | O |
| ATOM | 1892 | N | PRO | B | 142 | 36.542 | 4.326 | −31.248 | 1.00 | 28.52 | N |
| ATOM | 1893 | CA | PRO | B | 142 | 36.598 | 4.378 | −32.711 | 1.00 | 27.32 | C |
| ATOM | 1894 | CB | PRO | B | 142 | 37.670 | 5.442 | −33.014 | 1.00 | 27.65 | C |
| ATOM | 1895 | CG | PRO | B | 142 | 38.148 | 5.959 | −31.693 | 1.00 | 28.40 | C |
| ATOM | 1896 | CD | PRO | B | 142 | 37.694 | 5.006 | −30.627 | 1.00 | 28.58 | C |
| ATOM | 1897 | C | PRO | B | 142 | 36.985 | 3.035 | −33.321 | 1.00 | 26.18 | C |
| ATOM | 1898 | O | PRO | B | 142 | 37.911 | 2.386 | −32.839 | 1.00 | 26.49 | O |
| ATOM | 1899 | N | VAL | B | 143 | 36.253 | 2.626 | −34.355 | 1.00 | 25.37 | N |
| ATOM | 1900 | CA | VAL | B | 143 | 36.611 | 1.467 | −35.171 | 1.00 | 24.23 | C |
| ATOM | 1901 | CB | VAL | B | 143 | 35.620 | 0.293 | −34.992 | 1.00 | 23.88 | C |
| ATOM | 1902 | CG1 | VAL | B | 143 | 35.928 | −0.839 | −35.966 | 1.00 | 23.54 | C |
| ATOM | 1903 | CG2 | VAL | B | 143 | 35.653 | −0.199 | −33.552 | 1.00 | 23.89 | C |
| ATOM | 1904 | C | VAL | B | 143 | 36.651 | 1.938 | −36.620 | 1.00 | 22.87 | C |
| ATOM | 1905 | O | VAL | B | 143 | 35.726 | 2.591 | −37.089 | 1.00 | 22.35 | O |
| ATOM | 1906 | N | GLN | B | 144 | 37.744 | 1.618 | −37.308 | 1.00 | 22.92 | N |
| ATOM | 1907 | CA | GLN | B | 144 | 37.975 | 2.043 | −38.687 | 1.00 | 22.35 | C |
| ATOM | 1908 | CB | GLN | B | 144 | 39.439 | 2.439 | −38.893 | 1.00 | 21.86 | C |
| ATOM | 1909 | CG | GLN | B | 144 | 39.939 | 3.539 | −37.968 | 1.00 | 21.03 | C |
| ATOM | 1910 | CD | GLN | B | 144 | 41.423 | 3.795 | −38.080 | 1.00 | 20.16 | C |
| ATOM | 1911 | OE1 | GLN | B | 144 | 42.176 | 2.958 | −38.579 | 1.00 | 19.62 | O |
| ATOM | 1912 | NE2 | GLN | B | 144 | 41.852 | 4.967 | −37.608 | 1.00 | 20.19 | N |
| ATOM | 1913 | C | GLN | B | 144 | 37.616 | 0.933 | −39.657 | 1.00 | 22.41 | C |
| ATOM | 1914 | O | GLN | B | 144 | 37.902 | −0.234 | −39.401 | 1.00 | 22.21 | O |
| ATOM | 1915 | N | LEU | B | 145 | 37.003 | 1.320 | −40.774 | 1.00 | 23.01 | N |
| ATOM | 1916 | CA | LEU | B | 145 | 36.699 | 0.418 | −41.877 | 1.00 | 22.99 | C |
| ATOM | 1917 | CB | LEU | B | 145 | 35.254 | 0.602 | −42.329 | 1.00 | 22.10 | C |
| ATOM | 1918 | CG | LEU | B | 145 | 34.200 | 0.741 | −41.228 | 1.00 | 21.42 | C |
| ATOM | 1919 | CD1 | LEU | B | 145 | 32.822 | 0.907 | −41.854 | 1.00 | 20.86 | C |
| ATOM | 1920 | CD2 | LEU | B | 145 | 34.203 | −0.457 | −40.283 | 1.00 | 21.61 | C |
| ATOM | 1921 | C | LEU | B | 145 | 37.672 | 0.729 | −43.012 | 1.00 | 24.63 | C |
| ATOM | 1922 | O | LEU | B | 145 | 37.710 | 1.846 | −43.516 | 1.00 | 24.54 | O |
| ATOM | 1923 | N | TRP | B | 146 | 38.468 | −0.265 | −43.392 | 1.00 | 27.16 | N |
| ATOM | 1924 | CA | TRP | B | 146 | 39.424 | −0.149 | −44.490 | 1.00 | 29.00 | C |
| ATOM | 1925 | CB | TRP | B | 146 | 40.826 | −0.555 | −44.029 | 1.00 | 29.58 | C |
| ATOM | 1926 | CG | TRP | B | 146 | 41.462 | 0.407 | −43.048 | 1.00 | 30.99 | C |
| ATOM | 1927 | CD1 | TRP | B | 146 | 41.157 | 0.558 | −41.728 | 1.00 | 31.26 | C |
| ATOM | 1928 | NE1 | TRP | B | 146 | 41.950 | 1.525 | −41.160 | 1.00 | 31.38 | N |
| ATOM | 1929 | CE2 | TRP | B | 146 | 42.803 | 2.014 | −42.109 | 1.00 | 32.63 | C |
| ATOM | 1930 | CD2 | TRP | B | 146 | 42.529 | 1.332 | −43.315 | 1.00 | 32.50 | C |
| ATOM | 1931 | CE3 | TRP | B | 146 | 43.296 | 1.636 | −44.452 | 1.00 | 33.51 | C |
| ATOM | 1932 | CZ3 | TRP | B | 146 | 44.259 | 2.634 | −44.369 | 1.00 | 33.70 | C |
| ATOM | 1933 | CH2 | TRP | B | 146 | 44.512 | 3.302 | −43.152 | 1.00 | 33.79 | C |
| ATOM | 1934 | CZ2 | TRP | B | 146 | 43.797 | 3.005 | −42.012 | 1.00 | 33.77 | C |
| ATOM | 1935 | C | TRP | B | 146 | 38.961 | −1.060 | −45.620 | 1.00 | 29.66 | C |
| ATOM | 1936 | O | TRP | B | 146 | 38.685 | −2.239 | −45.379 | 1.00 | 31.61 | O |
| ATOM | 1937 | N | VAL | B | 147 | 38.853 | −0.505 | −46.830 | 1.00 | 30.50 | N |
| ATOM | 1938 | CA | VAL | B | 147 | 38.494 | −1.274 | −48.030 | 1.00 | 32.32 | C |
| ATOM | 1939 | CB | VAL | B | 147 | 37.044 | −1.004 | −48.510 | 1.00 | 31.12 | C |
| ATOM | 1940 | CG1 | VAL | B | 147 | 36.050 | −1.568 | −47.508 | 1.00 | 31.98 | C |
| ATOM | 1941 | CG2 | VAL | B | 147 | 36.786 | 0.470 | −48.756 | 1.00 | 31.31 | C |
| ATOM | 1942 | C | VAL | B | 147 | 39.467 | −1.000 | −49.179 | 1.00 | 33.64 | C |
| ATOM | 1943 | O | VAL | B | 147 | 39.990 | 0.107 | −49.312 | 1.00 | 32.54 | O |
| ATOM | 1944 | N | ASP | B | 148 | 39.709 | −2.027 | −49.998 | 1.00 | 36.72 | N |
| ATOM | 1945 | CA | ASP | B | 148 | 40.461 | −1.877 | −51.246 | 1.00 | 39.87 | C |
| ATOM | 1946 | CB | ASP | B | 148 | 40.968 | −3.231 | −51.746 | 1.00 | 40.74 | C |
| ATOM | 1947 | CG | ASP | B | 148 | 41.971 | −3.886 | −50.793 | 1.00 | 42.02 | C |
| ATOM | 1948 | OD1 | ASP | B | 148 | 42.629 | −3.189 | −49.981 | 1.00 | 42.62 | O |
| ATOM | 1949 | OD2 | ASP | B | 148 | 42.084 | −5.132 | −50.845 | 1.00 | 43.27 | O |
| ATOM | 1950 | C | ASP | B | 148 | 39.599 | −1.222 | −52.333 | 1.00 | 41.58 | C |
| ATOM | 1951 | O | ASP | B | 148 | 40.110 | −0.446 | −53.130 | 1.00 | 42.31 | O |
| ATOM | 1952 | N | SER | B | 149 | 38.306 | −1.545 | −52.364 | 1.00 | 44.60 | N |
| ATOM | 1953 | CA | SER | B | 149 | 37.369 | −1.026 | −53.372 | 1.00 | 45.30 | C |
| ATOM | 1954 | CB | SER | B | 149 | 37.000 | −2.129 | −54.379 | 1.00 | 47.22 | C |
| ATOM | 1955 | OG | SER | B | 149 | 38.115 | −2.925 | −54.743 | 1.00 | 49.26 | O |
| ATOM | 1956 | C | SER | B | 149 | 36.099 | −0.522 | −52.691 | 1.00 | 43.87 | C |
| ATOM | 1957 | O | SER | B | 149 | 35.530 | −1.214 | −51.860 | 1.00 | 44.30 | O |
| ATOM | 1958 | N | THR | B | 150 | 35.655 | 0.677 | −53.055 | 1.00 | 43.00 | N |
| ATOM | 1959 | CA | THR | B | 150 | 34.435 | 1.259 | −52.497 | 1.00 | 42.64 | C |
| ATOM | 1960 | CB | THR | B | 150 | 34.226 | 2.699 | −53.005 | 1.00 | 42.97 | C |
| ATOM | 1961 | OG1 | THR | B | 150 | 35.399 | 3.475 | −52.736 | 1.00 | 42.27 | O |
| ATOM | 1962 | CG2 | THR | B | 150 | 33.022 | 3.356 | −52.336 | 1.00 | 43.87 | C |
| ATOM | 1963 | C | THR | B | 150 | 33.214 | 0.399 | −52.859 | 1.00 | 42.89 | C |
| ATOM | 1964 | O | THR | B | 150 | 32.972 | 0.150 | −54.040 | 1.00 | 42.99 | O |
| ATOM | 1965 | N | PRO | B | 151 | 32.457 | −0.081 | −51.852 | 1.00 | 42.59 | N |
| ATOM | 1966 | CA | PRO | B | 151 | 31.223 | −0.814 | −52.171 | 1.00 | 43.06 | C |
| ATOM | 1967 | CB | PRO | B | 151 | 30.875 | −1.551 | −50.866 | 1.00 | 43.01 | C |
| ATOM | 1968 | CG | PRO | B | 151 | 31.704 | −0.949 | −49.796 | 1.00 | 43.19 | C |
| ATOM | 1969 | CD | PRO | B | 151 | 32.724 | −0.041 | −50.408 | 1.00 | 42.98 | C |
| ATOM | 1970 | C | PRO | B | 151 | 30.089 | 0.109 | −52.624 | 1.00 | 41.50 | C |
| ATOM | 1971 | O | PRO | B | 151 | 30.161 | 1.316 | −52.384 | 1.00 | 42.00 | O |
| ATOM | 1972 | N | PRO | B | 152 | 29.051 | −0.450 | −53.273 | 1.00 | 41.29 | N |
| ATOM | 1973 | CA | PRO | B | 152 | 27.990 | 0.394 | −53.856 | 1.00 | 41.70 | C |
| ATOM | 1974 | CB | PRO | B | 152 | 27.096 | −0.594 | −54.623 | 1.00 | 41.61 | C |
| ATOM | 1975 | CG | PRO | B | 152 | 27.505 | −1.974 | −54.180 | 1.00 | 41.65 | C |
| ATOM | 1976 | CD | PRO | B | 152 | 28.831 | −1.893 | −53.485 | 1.00 | 41.43 | C |
| ATOM | 1977 | C | PRO | B | 152 | 27.162 | 1.149 | −52.819 | 1.00 | 41.70 | C |
| ATOM | 1978 | O | PRO | B | 152 | 27.122 | 0.735 | −51.661 | 1.00 | 40.85 | O |
| ATOM | 1979 | N | PRO | B | 153 | 26.502 | 2.262 | −53.226 | 1.00 | 41.97 | N |
| ATOM | 1980 | CA | PRO | B | 153 | 25.611 | 2.990 | −52.299 | 1.00 | 39.64 | C |
| ATOM | 1981 | CB | PRO | B | 153 | 24.999 | 4.082 | −53.179 | 1.00 | 41.05 | C |
| ATOM | 1982 | CG | PRO | B | 153 | 25.990 | 4.290 | −54.269 | 1.00 | 41.56 | C |
| ATOM | 1983 | CD | PRO | B | 153 | 26.618 | 2.947 | −54.530 | 1.00 | 41.96 | C |
| ATOM | 1984 | C | PRO | B | 153 | 24.524 | 2.085 | −51.716 | 1.00 | 36.43 | C |
| ATOM | 1985 | O | PRO | B | 153 | 24.118 | 1.121 | −52.366 | 1.00 | 35.47 | O |
| ATOM | 1986 | N | GLY | B | 154 | 24.088 | 2.390 | −50.495 | 1.00 | 33.59 | N |
| ATOM | 1987 | CA | GLY | B | 154 | 23.125 | 1.554 | −49.766 | 1.00 | 31.05 | C |
| ATOM | 1988 | C | GLY | B | 154 | 23.747 | 0.395 | −48.994 | 1.00 | 29.45 | C |
| ATOM | 1989 | O | GLY | B | 154 | 23.039 | −0.356 | −48.326 | 1.00 | 29.15 | O |
| ATOM | 1990 | N | THR | B | 155 | 25.075 | 0.274 | −49.064 | 1.00 | 27.57 | N |
| ATOM | 1991 | CA | THR | B | 155 | 25.850 | −0.628 | −48.218 | 1.00 | 26.00 | C |
| ATOM | 1992 | CB | THR | B | 155 | 27.348 | −0.611 | −48.626 | 1.00 | 25.98 | C |
| ATOM | 1993 | OG1 | THR | B | 155 | 27.480 | −1.126 | −49.961 | 1.00 | 25.76 | O |
| ATOM | 1994 | CG2 | THR | B | 155 | 28.218 | −1.462 | −47.696 | 1.00 | 25.92 | C |
| ATOM | 1995 | C | THR | B | 155 | 25.700 | −0.215 | −46.759 | 1.00 | 24.18 | C |
| ATOM | 1996 | O | THR | B | 155 | 25.616 | 0.980 | −46.446 | 1.00 | 23.53 | O |
| ATOM | 1997 | N | ARG | B | 155 | 25.677 | −1.214 | −45.877 | 1.00 | 23.67 | N |
| ATOM | 1998 | CA | ARG | B | 156 | 25.473 | −1.012 | −44.439 | 1.00 | 22.76 | C |
| ATOM | 1999 | CB | ARG | B | 156 | 24.065 | −1.484 | −44.045 | 1.00 | 22.35 | C |
| ATOM | 2000 | CG | ARG | B | 156 | 23.006 | −0.392 | −44.136 | 1.00 | 22.39 | C |
| ATOM | 2001 | CD | ARG | B | 156 | 21.669 | −0.880 | −44.669 | 1.00 | 22.20 | C |
| ATOM | 2002 | NE | ARG | B | 156 | 20.973 | −1.835 | −43.798 | 1.00 | 22.16 | N |
| ATOM | 2003 | CZ | ARG | B | 156 | 20.081 | −1.524 | −42.845 | 1.00 | 22.37 | C |
| ATOM | 2004 | NH1 | ARG | B | 156 | 19.758 | −0.260 | −42.557 | 1.00 | 22.14 | N |
| ATOM | 2005 | NH2 | ARG | B | 156 | 19.509 | −2.502 | −42.152 | 1.00 | 22.42 | N |
| ATOM | 2006 | C | ARG | B | 156 | 26.542 | −1.720 | −43.609 | 1.00 | 22.44 | C |
| ATOM | 2007 | O | ARG | B | 156 | 27.146 | −2.697 | −44.063 | 1.00 | 23.05 | O |
| ATOM | 2008 | N | VAL | B | 157 | 26.742 | −1.220 | −42.385 | 1.00 | 21.80 | N |
| ATOM | 2009 | CA | VAL | B | 157 | 27.744 | −1.723 | −41.453 | 1.00 | 21.83 | C |
| ATOM | 2010 | CB | VAL | B | 157 | 28.829 | −0.662 | −41.161 | 1.00 | 21.59 | C |
| ATOM | 2011 | CG1 | VAL | B | 157 | 30.014 | −1.281 | −40.426 | 1.00 | 21.48 | C |
| ATOM | 2012 | CG2 | VAL | B | 157 | 29.292 | −0.009 | −42.455 | 1.00 | 21.69 | C |
| ATOM | 2013 | C | VAL | B | 157 | 27.055 | −2.110 | −40.143 | 1.00 | 21.10 | C |
| ATOM | 2014 | O | VAL | B | 157 | 26.514 | −1.245 | −39.448 | 1.00 | 20.81 | O |
| ATOM | 2015 | N | ARG | B | 158 | 27.092 | −3.405 | −39.812 | 1.00 | 19.72 | N |
| ATOM | 2016 | CA | ARG | B | 158 | 26.458 | −3.928 | −38.608 | 1.00 | 19.20 | C |
| ATOM | 2017 | CB | ARG | B | 158 | 25.591 | −5.128 | −38.959 | 1.00 | 18.71 | C |
| ATOM | 2018 | CG | ARG | B | 158 | 24.721 | −5.661 | −37.818 | 1.00 | 18.43 | C |
| ATOM | 2019 | CD | ARG | B | 158 | 23.869 | −6.835 | −38.278 | 1.00 | 17.94 | C |
| ATOM | 2020 | NE | ARG | B | 158 | 22.996 | −6.429 | −39.382 | 1.00 | 17.34 | N |
| ATOM | 2021 | CZ | ARG | B | 158 | 21.816 | −5.820 | −39.267 | 1.00 | 17.10 | C |
| ATOM | 2022 | NH1 | ARG | B | 158 | 21.275 | −5.560 | −38.075 | 1.00 | 18.01 | N |
| ATOM | 2023 | NH2 | ARG | B | 158 | 21.159 | −5.480 | −40.363 | 1.00 | 16.50 | N |
| ATOM | 2024 | C | ARG | B | 158 | 27.497 | −4.353 | −37.585 | 1.00 | 18.81 | C |
| ATOM | 2025 | O | ARG | B | 158 | 28.513 | −4.923 | −37.948 | 1.00 | 19.54 | O |
| ATOM | 2026 | N | ALA | B | 159 | 27.227 | −4.062 | −36.314 | 1.00 | 18.43 | N |
| ATOM | 2027 | CA | ALA | B | 159 | 28.021 | −4.573 | −35.200 | 1.00 | 18.21 | C |
| ATOM | 2028 | CB | ALA | B | 159 | 28.610 | −3.422 | −34.397 | 1.00 | 18.38 | C |
| ATOM | 2029 | C | ALA | B | 159 | 27.117 | −5.440 | −34.322 | 1.00 | 18.05 | C |
| ATOM | 2030 | O | ALA | B | 159 | 25.957 | −5.110 | −34.095 | 1.00 | 16.75 | O |
| ATOM | 2031 | N | MET | B | 160 | 27.661 | −6.553 | −33.843 | 1.00 | 18.72 | N |
| ATOM | 2032 | CA | MET | B | 160 | 26.932 | −7.492 | −32.973 | 1.00 | 19.25 | C |
| ATOM | 2033 | CB | MET | B | 160 | 26.256 | −8.581 | −33.817 | 1.00 | 19.74 | C |
| ATOM | 2034 | CG | MET | B | 160 | 25.625 | −9.739 | −33.039 | 1.00 | 20.78 | C |
| ATOM | 2035 | SD | MET | B | 160 | 24.870 | −11.055 | −34.060 | 1.00 | 21.68 | S |
| ATOM | 2036 | CE | MET | B | 160 | 23.762 | −10.205 | −35.178 | 1.00 | 21.51 | C |
| ATOM | 2037 | C | MET | B | 160 | 27.918 | −8.090 | −31.973 | 1.00 | 18.68 | C |
| ATOM | 2038 | O | MET | B | 160 | 29.048 | −8.406 | −32.341 | 1.00 | 18.42 | O |
| ATOM | 2039 | N | ALA | B | 161 | 27.475 | −8.252 | −30.727 | 1.00 | 19.20 | N |
| ATOM | 2040 | CA | ALA | B | 161 | 28.294 | −8.896 | −29.692 | 1.00 | 20.03 | C |
| ATOM | 2041 | CB | ALA | B | 161 | 28.195 | −8.137 | −28.389 | 1.00 | 19.40 | C |
| ATOM | 2042 | C | ALA | B | 161 | 27.880 | −10.363 | −29.490 | 1.00 | 20.46 | C |
| ATOM | 2043 | O | ALA | B | 161 | 26.700 | −10.712 | −29.598 | 1.00 | 20.08 | O |
| ATOM | 2044 | N | ILE | B | 162 | 28.871 | −11.216 | −29.225 | 1.00 | 20.98 | N |
| ATOM | 2045 | CA | ILE | B | 162 | 28.642 | −12.594 | −28.747 | 1.00 | 22.26 | C |
| ATOM | 2046 | CB | ILE | B | 162 | 28.702 | −13.642 | −29.880 | 1.00 | 22.38 | C |
| ATOM | 2047 | CG1 | ILE | B | 162 | 30.093 | −13.707 | −30.538 | 1.00 | 22.76 | C |
| ATOM | 2048 | CD1 | ILE | B | 162 | 30.271 | −14.921 | −31.430 | 1.00 | 22.90 | C |
| ATOM | 2049 | CO2 | ILE | B | 162 | 27.660 | −13.349 | −30.962 | 1.00 | 22.54 | C |
| ATOM | 2050 | C | ILE | B | 162 | 29.676 | −12.946 | −27.690 | 1.00 | 23.42 | C |
| ATOM | 2051 | O | ILE | B | 162 | 30.742 | −12.345 | −27.643 | 1.00 | 23.62 | O |
| ATOM | 2052 | N | TYR | B | 163 | 29.371 | −13.903 | −26.826 | 1.00 | 26.27 | N |
| ATOM | 2053 | CA | TYR | B | 163 | 30.337 | −14.360 | −25.811 | 1.00 | 28.08 | C |
| ATOM | 2054 | CB | TYR | B | 163 | 29.621 | −15.054 | −24.676 | 1.00 | 28.35 | C |
| ATOM | 2055 | CG | TYR | B | 163 | 28.718 | −14.139 | −23.883 | 1.00 | 29.07 | C |
| ATOM | 2056 | CD1 | TYR | B | 163 | 29.247 | −13.078 | −23.164 | 1.00 | 29.95 | C |
| ATOM | 2057 | CE1 | TYR | B | 163 | 28.432 | −12.224 | −22.428 | 1.00 | 30.28 | C |
| ATOM | 2058 | CZ | TYR | B | 163 | 27.064 | −12.437 | −22.410 | 1.00 | 30.39 | C |
| ATOM | 2059 | OH | TYR | B | 163 | 26.261 | −11.585 | −21.684 | 1.00 | 30.39 | O |
| ATOM | 2060 | CE2 | TYR | B | 163 | 26.509 | −13.485 | −23.128 | 1.00 | 29.74 | C |
| ATOM | 2061 | CD2 | TYR | B | 163 | 27.330 | −14.327 | −23.854 | 1.00 | 29.44 | C |
| ATOM | 2062 | C | TYR | B | 163 | 31.372 | −15.281 | −26.452 | 1.00 | 30.45 | C |
| ATOM | 2063 | O | TYR | B | 163 | 31.049 | −16.087 | −27.328 | 1.00 | 29.87 | O |
| ATOM | 2064 | N | LYS | B | 164 | 32.628 | −15.133 | −26.033 | 1.00 | 34.62 | N |
| ATOM | 2065 | CA | LYS | B | 164 | 33.749 | −15.879 | −26.620 | 1.00 | 37.51 | C |
| ATOM | 2066 | CB | LYS | B | 164 | 35.071 | −15.151 | −26.326 | 1.00 | 39.87 | C |
| ATOM | 2067 | CG | LYS | B | 164 | 36.308 | −15.713 | −27.014 | 1.00 | 41.13 | C |
| ATOM | 2068 | CD | LYS | B | 164 | 37.540 | −14.920 | −26.580 | 1.00 | 41.80 | C |
| ATOM | 2069 | CE | LYS | B | 164 | 38.837 | −15.615 | −26.837 | 1.00 | 41.68 | C |
| ATOM | 2070 | NZ | LYS | B | 164 | 39.926 | −15.152 | −25.882 | 1.00 | 40.91 | N |
| ATOM | 2071 | C | LYS | B | 164 | 33.819 | −17.314 | −26.111 | 1.00 | 38.78 | C |
| ATOM | 2072 | O | LYS | B | 164 | 34.142 | −18.210 | −26.875 | 1.00 | 39.22 | O |
| ATOM | 2073 | N | GLN | B | 165 | 33.526 | −17.544 | −24.826 | 1.00 | 40.84 | N |
| ATOM | 2074 | CA | GLN | B | 165 | 33.640 | −18.875 | −24.225 | 1.00 | 43.01 | C |
| ATOM | 2075 | CB | GLN | B | 165 | 33.540 | −18.785 | −22.706 | 1.00 | 43.28 | C |
| ATOM | 2076 | CG | GLN | B | 165 | 34.694 | −18.029 | −22.060 | 1.00 | 43.55 | C |
| ATOM | 2077 | CD | GLN | B | 165 | 34.570 | −17.922 | −20.548 | 1.00 | 43.97 | C |
| ATOM | 2078 | OE1 | GLN | B | 165 | 33.516 | −18.166 | −19.954 | 1.00 | 45.06 | O |
| ATOM | 2079 | NE2 | GLN | B | 165 | 35.670 | −17.572 | −19.908 | 1.00 | 44.34 | N |
| ATOM | 2080 | C | GLN | B | 165 | 32.571 | −19.830 | −24.766 | 1.00 | 44.28 | C |
| ATOM | 2081 | O | GLN | B | 165 | 31.369 | −19.494 | −24.796 | 1.00 | 46.39 | O |
| ATOM | 2082 | N | SER | B | 166 | 33.010 | −21.023 | −25.178 | 1.00 | 45.00 | N |
| ATOM | 2083 | CA | SER | B | 166 | 32.145 | −22.005 | −25.849 | 1.00 | 45.41 | C |
| ATOM | 2084 | CB | SER | B | 166 | 32.938 | −23.275 | −26.195 | 1.00 | 46.16 | C |
| ATOM | 2085 | OG | SER | B | 166 | 33.668 | −23.753 | −25.077 | 1.00 | 48.11 | O |
| ATOM | 2086 | C | SER | B | 166 | 30.878 | −22.373 | −25.068 | 1.00 | 43.67 | C |
| ATOM | 2087 | O | SER | B | 166 | 29.825 | −22.603 | −25.668 | 1.00 | 44.65 | O |
| ATOM | 2088 | N | GLN | B | 167 | 30.982 | −22.421 | −23.740 | 1.00 | 43.34 | N |
| ATOM | 2089 | CA | GLN | B | 167 | 29.828 | −22.737 | −22.886 | 1.00 | 43.37 | C |
| ATOM | 2090 | CB | GLN | B | 167 | 30.274 | −23.203 | −21.482 | 1.00 | 44.46 | C |
| ATOM | 2091 | CG | GLN | B | 167 | 30.826 | −22.125 | −20.540 | 1.00 | 45.31 | C |
| ATOM | 2092 | CD | GLN | B | 167 | 32.309 | −21.843 | −20.728 | 1.00 | 46.26 | C |
| ATOM | 2093 | OE1 | GLN | B | 167 | 32.905 | −22.177 | −21.756 | 1.00 | 45.68 | O |
| ATOM | 2094 | NE2 | GLN | B | 167 | 32.910 | −21.203 | −19.729 | 1.00 | 46.61 | N |
| ATOM | 2095 | C | GLN | B | 167 | 28.779 | −21.616 | −22.775 | 1.00 | 41.40 | C |
| ATOM | 2096 | O | GLN | B | 167 | 27.644 | −21.880 | −22.363 | 1.00 | 40.53 | O |
| ATOM | 2097 | N | HIS | B | 168 | 29.159 | −20.381 | −23.119 | 1.00 | 39.27 | N |
| ATOM | 2098 | CA | HIS | B | 168 | 28.242 | −19.230 | −23.112 | 1.00 | 37.67 | C |
| ATOM | 2099 | CB | HIS | B | 168 | 28.841 | −18.066 | −22.327 | 1.00 | 37.76 | C |
| ATOM | 2100 | CG | HIS | B | 168 | 29.065 | −18.357 | −20.879 | 1.00 | 37.82 | C |
| ATOM | 2101 | ND1 | HIS | B | 168 | 28.056 | −18.744 | −20.029 | 1.00 | 38.12 | N |
| ATOM | 2102 | CE1 | HIS | B | 168 | 28.556 | −18.933 | −18.818 | 1.00 | 38.63 | C |
| ATOM | 2103 | NE2 | HIS | B | 168 | 29.854 | −18.701 | −18.861 | 1.00 | 37.97 | N |
| ATOM | 2104 | CD2 | HIS | B | 168 | 30.198 | −18.361 | −20.145 | 1.00 | 37.44 | C |
| ATOM | 2105 | C | HIS | B | 168 | 27.822 | −18.725 | −24.511 | 1.00 | 36.72 | C |
| ATOM | 2106 | O | HIS | B | 168 | 26.965 | −17.845 | −24.588 | 1.00 | 38.86 | O |
| ATOM | 2107 | N | MET | B | 169 | 28.404 | −19.269 | −25.582 | 1.00 | 35.81 | N |
| ATOM | 2108 | CA | MET | B | 169 | 28.224 | −18.723 | −26.941 | 1.00 | 35.92 | C |
| ATOM | 2109 | CB | MET | B | 169 | 29.048 | −19.523 | −27.974 | 1.00 | 36.95 | C |
| ATOM | 2110 | CG | MET | B | 169 | 30.500 | −19.104 | −28.191 | 1.00 | 38.56 | C |
| ATOM | 2111 | SD | MET | B | 169 | 31.157 | −19.503 | −29.835 | 1.00 | 39.27 | S |
| ATOM | 2112 | CE | MET | B | 169 | 32.823 | −18.922 | −29.686 | 1.00 | 39.36 | C |
| ATOM | 2113 | C | MET | B | 169 | 26.766 | −18.603 | −27.438 | 1.00 | 33.75 | C |
| ATOM | 2114 | O | MET | B | 169 | 26.457 | −17.713 | −28.245 | 1.00 | 33.14 | O |
| ATOM | 2115 | N | THR | B | 170 | 25.893 | −19.480 | −26.956 | 1.00 | 31.66 | N |
| ATOM | 2116 | CA | THR | B | 170 | 24.485 | −19.486 | −27.358 | 1.00 | 31.69 | C |
| ATOM | 2117 | CB | THR | B | 170 | 23.841 | −20.878 | −27.124 | 1.00 | 31.75 | C |
| ATOM | 2118 | OG1 | THR | B | 170 | 23.881 | −21.222 | −25.733 | 1.00 | 30.95 | O |
| ATOM | 2119 | CG2 | THR | B | 170 | 24.573 | −21.943 | −27.897 | 1.00 | 31.17 | C |
| ATOM | 2120 | C | THR | B | 170 | 23.638 | −18.436 | −26.625 | 1.00 | 31.32 | C |
| ATOM | 2121 | O | THR | B | 170 | 22.539 | −18.126 | −27.084 | 1.00 | 30.91 | O |
| ATOM | 2122 | N | GLU | B | 171 | 24.132 | −17.908 | −25.490 | 1.00 | 31.40 | N |
| ATOM | 2123 | CA | GLU | B | 171 | 23.422 | −16.869 | −24.771 | 1.00 | 32.18 | C |
| ATOM | 2124 | CB | GLU | B | 171 | 23.970 | −16.694 | −23.358 | 1.00 | 35.38 | C |
| ATOM | 2125 | CG | GLU | B | 171 | 23.878 | −17.857 | −22.412 | 1.00 | 38.53 | C |
| ATOM | 2126 | CD | GLU | B | 171 | 23.919 | −17.348 | −20.955 | 1.00 | 41.95 | C |
| ATOM | 2127 | OE1 | GLU | B | 171 | 24.962 | −17.466 | −20.257 | 1.00 | 44.22 | O |
| ATOM | 2128 | OE2 | GLU | B | 171 | 22.908 | −16.744 | −20.545 | 1.00 | 46.79 | O |
| ATOM | 2129 | C | GLU | B | 171 | 23.520 | −15.532 | −25.492 | 1.00 | 30.27 | C |
| ATOM | 2130 | O | GLU | B | 171 | 24.568 | −15.181 | −26.059 | 1.00 | 29.68 | O |
| ATOM | 2131 | N | VAL | B | 172 | 22.427 | −14.772 | −25.450 | 1.00 | 28.29 | N |
| ATOM | 2132 | CA | VAL | B | 172 | 22.363 | −13.463 | −26.084 | 1.00 | 27.10 | C |
| ATOM | 2133 | CB | VAL | B | 172 | 20.910 | −12.997 | −26.293 | 1.00 | 27.55 | C |
| ATOM | 2134 | CG1 | VAL | B | 172 | 20.860 | −11.572 | −26.858 | 1.00 | 27.95 | C |
| ATOM | 2135 | CG2 | VAL | B | 172 | 20.157 | −13.988 | −27.190 | 1.00 | 28.11 | C |
| ATOM | 2136 | C | VAL | B | 172 | 23.092 | −12.464 | −25.200 | 1.00 | 25.78 | C |
| ATOM | 2137 | O | VAL | B | 172 | 22.787 | −12.349 | −24.006 | 1.00 | 24.85 | O |
| ATOM | 2138 | N | VAL | B | 173 | 24.051 | −11.749 | −25.790 | 1.00 | 24.53 | N |
| ATOM | 2139 | CA | VAL | B | 173 | 24.739 | −10.673 | −25.092 | 1.00 | 24.71 | C |
| ATOM | 2140 | CE | VAL | B | 173 | 26.017 | −10.211 | −25.827 | 1.00 | 24.16 | C |
| ATOM | 2141 | CG1 | VAL | B | 173 | 26.629 | −8.990 | −25.138 | 1.00 | 24.00 | C |
| ATOM | 2142 | CG2 | VAL | B | 173 | 27.043 | −11.336 | −25.892 | 1.00 | 23.88 | C |
| ATOM | 2143 | C | VAL | B | 173 | 23.773 | −9.503 | −24.947 | 1.00 | 24.29 | C |
| ATOM | 2144 | O | VAL | B | 173 | 23.372 | −8.916 | −25.948 | 1.00 | 22.82 | O |
| ATOM | 2145 | N | ARG | B | 174 | 23.384 | −9.216 | −23.717 | 1.00 | 24.68 | N |
| ATOM | 2146 | CA | ARG | B | 174 | 22.497 | −8.112 | −23.404 | 1.00 | 26.96 | C |
| ATOM | 2147 | CB | ARG | B | 174 | 21.041 | −8.561 | −23.103 | 1.00 | 27.76 | C |
| ATOM | 2148 | CG | ARG | B | 174 | 20.935 | −9.675 | −22.085 | 1.00 | 29.36 | C |
| ATOM | 2149 | CD | ARG | B | 174 | 19.462 | −9.962 | −21.748 | 1.00 | 30.41 | C |
| ATOM | 2150 | NE | ARG | B | 174 | 18.881 | −10.999 | −22.606 | 1.00 | 32.00 | N |
| ATOM | 2151 | CZ | ARG | B | 174 | 18.193 | −10.816 | −23.742 | 1.00 | 32.76 | C |
| ATOM | 2152 | NH1 | ARG | B | 174 | 17.930 | −9.592 | −24.220 | 1.00 | 32.98 | N |
| ATOM | 2153 | NH2 | ARG | B | 174 | 17.698 | −11.882 | −24.376 | 1.00 | 32.43 | N |
| ATOM | 2154 | C | ARG | B | 174 | 23.065 | −7.366 | −22.209 | 1.00 | 26.16 | C |
| ATOM | 2155 | O | ARG | B | 174 | 23.933 | −7.862 | −21.501 | 1.00 | 24.98 | O |
| ATOM | 2156 | N | ARG | B | 175 | 22.557 | −6.156 | −21.993 | 1.00 | 26.02 | N |
| ATOM | 2157 | CA | ARG | B | 175 | 22.889 | −5.373 | −20.819 | 1.00 | 26.25 | C |
| ATOM | 2158 | CB | ARG | B | 175 | 22.334 | −3.956 | −20.978 | 1.00 | 25.64 | C |
| ATOM | 2159 | CG | ARG | B | 175 | 23.155 | −3.074 | −21.911 | 1.00 | 25.38 | C |
| ATOM | 2160 | CD | ARG | B | 175 | 23.417 | −1.686 | −21.353 | 1.00 | 24.96 | C |
| ATOM | 2161 | NE | ARG | B | 175 | 22.283 | −0.777 | −21.431 | 1.00 | 23.83 | N |
| ATOM | 2162 | CZ | ARG | B | 175 | 22.267 | 0.463 | −20.941 | 1.00 | 23.49 | C |
| ATOM | 2163 | NH1 | ARG | B | 175 | 21.168 | 1.202 | −21.071 | 1.00 | 22.53 | N |
| ATOM | 2164 | NH2 | ARG | B | 175 | 23.299 | 0.970 | −20.287 | 1.00 | 24.44 | N |
| ATOM | 2165 | C | ARG | B | 175 | 22.354 | −6.019 | −19.540 | 1.00 | 26.52 | C |
| ATOM | 2166 | O | ARG | B | 175 | 21.353 | −6.705 | −19.569 | 1.00 | 27.88 | O |
| ATOM | 2167 | N | CYS | B | 176 | 23.039 | −5.791 | −18.420 | 1.00 | 26.52 | N |
| ATOM | 2168 | CA | CYS | B | 176 | 22.640 | −6.365 | −17.142 | 1.00 | 27.68 | C |
| ATOM | 2169 | CB | CYS | B | 175 | 23.764 | −6.250 | −16.115 | 1.00 | 27.58 | C |
| ATOM | 2170 | SG | CYS | B | 176 | 24.086 | −4.580 | −15.550 | 1.00 | 27.40 | S |
| ATOM | 2171 | C | CYS | B | 176 | 21.386 | −5.625 | −16.663 | 1.00 | 27.87 | C |
| ATOM | 2172 | O | CYS | B | 176 | 21.189 | −4.482 | −17.026 | 1.00 | 29.88 | O |
| ATOM | 2173 | N | PRO | B | 177 | 20.614 | −6.230 | −15.751 | 1.00 | 27.14 | N |
| ATOM | 2174 | CA | PRO | B | 177 | 19.418 | −5.555 | −15.222 | 1.00 | 26.75 | C |
| ATOM | 2175 | CB | PRO | B | 177 | 18.961 | −6.490 | −14.100 | 1.00 | 26.22 | C |
| ATOM | 2176 | CG | PRO | B | 177 | 19.492 | −7.833 | −14.471 | 1.00 | 26.87 | C |
| ATOM | 2177 | CD | PRO | B | 177 | 20.807 | −7.563 | −15.128 | 1.00 | 26.38 | C |
| ATOM | 2178 | C | PRO | B | 177 | 19.632 | −4.130 | −14.678 | 1.00 | 26.75 | C |
| ATOM | 2179 | O | PRO | B | 177 | 18.814 | −3.262 | −14.933 | 1.00 | 26.12 | O |
| ATOM | 2180 | N | HIS | B | 178 | 20.741 | −3.892 | −13.984 | 1.00 | 27.08 | N |
| ATOM | 2181 | CA | HIS | B | 178 | 21.062 | −2.538 | −13.519 | 1.00 | 26.58 | C |
| ATOM | 2182 | CB | HIS | B | 178 | 22.304 | −2.522 | −12.604 | 1.00 | 25.41 | C |
| ATOM | 2183 | CG | HIS | B | 178 | 22.985 | −1.193 | −12.545 | 1.00 | 25.07 | C |
| ATOM | 2184 | ND1 | HIS | B | 178 | 22.405 | −0.089 | −11.958 | 1.00 | 24.45 | N |
| ATOM | 2185 | CE1 | HIS | B | 178 | 23.228 | 0.940 | −12.055 | 1.00 | 25.36 | C |
| ATOM | 2166 | NE2 | HIS | B | 178 | 24.312 | 0.549 | −12.707 | 1.00 | 25.48 | N |
| ATOM | 2187 | CD2 | HIS | B | 178 | 24.186 | −0.782 | −13.023 | 1.00 | 25.11 | C |
| ATOM | 2188 | C | HIS | B | 178 | 21.241 | −1.547 | −14.672 | 1.00 | 26.61 | C |
| ATOM | 2189 | O | HIS | B | 178 | 20.651 | −0.472 | −14.655 | 1.00 | 27.02 | O |
| ATOM | 2190 | N | HIS | B | 179 | 22.074 | −1.897 | −15.649 | 1.00 | 27.75 | N |
| ATOM | 2191 | CA | HIS | B | 179 | 22.344 | −0.992 | −16.777 | 1.00 | 28.42 | C |
| ATOM | 2192 | CB | HIS | B | 179 | 23.599 | −1.423 | −17.565 | 1.00 | 28.61 | C |
| ATOM | 2193 | CG | HIS | B | 179 | 24.881 | −0.896 | −16.991 | 1.00 | 27.53 | C |
| ATOM | 2194 | ND1 | HIS | B | 179 | 25.901 | −1.710 | −16.553 | 1.00 | 26.13 | N |
| ATOM | 2195 | CE1 | HIS | B | 179 | 26.887 | −0.963 | −16.096 | 1.00 | 26.69 | C |
| ATOM | 2196 | NE1 | HIS | B | 179 | 26.551 | 0.307 | −16.235 | 1.00 | 26.40 | N |
| ATOM | 2197 | CD2 | HIS | B | 179 | 25.300 | 0.375 | −16.790 | 1.00 | 26.31 | C |
| ATOM | 2198 | C | HIS | B | 179 | 21.118 | −0.830 | −17.692 | 1.00 | 28.78 | C |
| ATOM | 2199 | O | HIS | B | 179 | 20.837 | 0.276 | −18.165 | 1.00 | 27.08 | O |
| ATOM | 2200 | N | HIS | B | 180 | 20.377 | −1.914 | −17.900 | 1.00 | 29.70 | N |
| ATOM | 2201 | CA | HIS | B | 180 | 19.076 | −1.844 | −18.570 | 1.00 | 32.29 | C |
| ATOM | 2202 | CB | GLU | B | 180 | 18.386 | −3.216 | −18.548 | 1.00 | 33.07 | C |
| ATOM | 2203 | CG | GLU | B | 180 | 17.040 | −3.235 | −19.265 | 1.00 | 35.28 | C |
| ATOM | 2204 | CD | GLU | B | 180 | 16.416 | −4.611 | −19.383 | 1.00 | 37.32 | C |
| ATOM | 2205 | OE1 | GLU | B | 180 | 17.093 | −5.633 | −19.134 | 1.00 | 37.82 | O |
| ATOM | 2206 | OE2 | GLU | B | 180 | 15.224 | −4.664 | −19.758 | 1.00 | 39.93 | O |
| ATOM | 2207 | C | GLU | B | 180 | 18.132 | −0.776 | −17.992 | 1.00 | 33.41 | C |
| ATOM | 2208 | O | GLU | B | 180 | 17.425 | −0.110 | −18.745 | 1.00 | 32.41 | O |
| ATOM | 2209 | N | ARG | B | 181 | 18.118 | −0.632 | −16.666 | 1.00 | 36.54 | N |
| ATOM | 2210 | CA | ARG | B | 181 | 17.213 | 0.306 | −15.983 | 1.00 | 39.64 | C |
| ATOM | 2211 | CB | ARG | B | 181 | 16.696 | −0.324 | −14.676 | 1.00 | 43.34 | C |
| ATOM | 2212 | CG | ARG | B | 181 | 15.841 | −1.566 | −14.929 | 1.00 | 47.46 | C |
| ATOM | 2213 | CD | ARG | B | 181 | 15.212 | −2.152 | −13.572 | 1.00 | 49.95 | C |
| ATOM | 2214 | NE | ARG | B | 181 | 14.252 | −3.213 | −13.999 | 1.00 | 52.29 | N |
| ATOM | 2215 | CZ | ARG | B | 181 | 13.017 | −3.026 | −14.463 | 1.00 | 54.22 | C |
| ATOM | 2216 | NH1 | ARG | B | 181 | 12.542 | −1.801 | −14.725 | 1.00 | 54.30 | N |
| ATOM | 2217 | NH2 | ARG | B | 181 | 12.245 | −4.083 | −14.741 | 1.00 | 54.89 | N |
| ATOM | 2218 | C | ARG | B | 181 | 17.813 | 1.696 | −15.726 | 1.00 | 38.97 | C |
| ATOM | 2219 | O | ARG | B | 181 | 17.135 | 2.556 | −15.166 | 1.00 | 39.24 | O |
| ATOM | 2220 | N | CYS | B | 182 | 19.065 | 1.921 | −16.129 | 1.00 | 39.25 | N |
| ATOM | 2221 | CA | CYS | B | 182 | 19.733 | 3.222 | −15.940 | 1.00 | 38.57 | C |
| ATOM | 2222 | CB | CYS | B | 182 | 21.232 | 3.152 | −16.277 | 1.00 | 39.46 | C |
| ATOM | 2223 | SG | CYS | B | 182 | 22.253 | 2.471 | −14.967 | 1.00 | 42.36 | S |
| ATOM | 2224 | C | CYS | B | 182 | 19.124 | 4.315 | −16.791 | 1.00 | 35.98 | C |
| ATOM | 2225 | O | CYS | B | 182 | 18.634 | 4.055 | −17.880 | 1.00 | 35.92 | O |
| ATOM | 2226 | N | SER | B | 183 | 19.230 | 5.544 | −16.297 | 1.00 | 35.57 | N |
| ATOM | 2227 | CA | SER | B | 183 | 18.850 | 6.736 | −17.046 | 1.00 | 35.19 | C |
| ATOM | 2228 | CB | SER | B | 183 | 18.376 | 7.839 | −16.091 | 1.00 | 35.88 | C |
| ATOM | 2229 | OG | SER | B | 183 | 17.217 | 7.429 | −15.390 | 1.00 | 35.41 | O |
| ATOM | 2230 | C | SER | B | 183 | 20.042 | 7.204 | −17.871 | 1.00 | 34.09 | C |
| ATOM | 2231 | O | SER | B | 183 | 20.637 | 8.248 | −17.598 | 1.00 | 34.07 | O |
| ATOM | 2232 | N | ASP | B | 184 | 20.376 | 6.416 | −18.891 | 1.00 | 32.97 | N |
| ATOM | 2233 | CA | ASP | B | 184 | 21.470 | 6.733 | −19.805 | 1.00 | 31.22 | C |
| ATOM | 2234 | CB | ASP | B | 184 | 22.676 | 5.821 | −19.521 | 1.00 | 31.28 | C |
| ATOM | 2235 | CG | ASP | B | 184 | 22.378 | 4.334 | −19.730 | 1.00 | 31.50 | C |
| ATOM | 2236 | OD1 | ASP | B | 184 | 21.385 | 3.951 | −20.408 | 1.00 | 29.03 | O |
| ATOM | 2237 | OD2 | ASP | B | 184 | 23.170 | 3.522 | −19.205 | 1.00 | 32.26 | O |
| ATOM | 2238 | C | ASP | B | 184 | 21.033 | 6.642 | −21.263 | 1.00 | 29.89 | C |
| ATOM | 2239 | O | ASP | B | 184 | 21.864 | 6.449 | −22.145 | 1.00 | 29.99 | O |
| ATOM | 2240 | N | SER | B | 185 | 19.731 | 6.791 | −21.511 | 1.00 | 30.08 | N |
| ATOM | 2241 | CA | SER | B | 185 | 19.196 | 6.718 | −22.868 | 1.00 | 29.75 | C |
| ATOM | 2242 | CB | SER | B | 185 | 17.669 | 6.659 | −22.843 | 1.00 | 30.53 | C |
| ATOM | 2243 | OG | SER | B | 185 | 17.133 | 6.601 | −24.155 | 1.00 | 31.58 | O |
| ATOM | 2244 | C | SER | B | 185 | 19.646 | 7.911 | −23.716 | 1.00 | 29.53 | C |
| ATOM | 2245 | O | SER | B | 185 | 19.810 | 9.028 | −23.197 | 1.00 | 27.89 | O |
| ATOM | 2246 | N | ASP | B | 186 | 19.842 | 7.653 | −25.016 | 1.00 | 29.00 | N |
| ATOM | 2247 | CA | ASP | B | 186 | 20.096 | 8.698 | −26.013 | 1.00 | 28.65 | C |
| ATOM | 2248 | CB | ASP | B | 186 | 21.358 | 8.366 | −26.840 | 1.00 | 29.13 | C |
| ATOM | 2249 | CG | ASP | B | 186 | 21.247 | 7.052 | −27.654 | 1.00 | 29.06 | C |
| ATOM | 2250 | OD1 | ASP | B | 186 | 20.260 | 6.294 | −27.520 | 1.00 | 28.61 | O |
| ATOM | 2251 | OD2 | ASP | B | 186 | 22.141 | 6.799 | −28.480 | 1.00 | 29.55 | O |
| ATOM | 2252 | C | ASP | B | 186 | 18.867 | 8.958 | −26.914 | 1.00 | 29.06 | C |
| ATOM | 2253 | O | ASP | B | 186 | 18.969 | 9.655 | −27.924 | 1.00 | 30.48 | O |
| ATOM | 2254 | N | GLY | B | 187 | 17.713 | 8.389 | −26.557 | 1.00 | 29.15 | N |
| ATOM | 2255 | CA | GLY | B | 187 | 16.505 | 8.505 | −27.375 | 1.00 | 29.51 | C |
| ATOM | 2256 | C | GLY | B | 187 | 16.439 | 7.594 | −28.599 | 1.00 | 29.95 | C |
| ATOM | 2257 | O | GLY | B | 187 | 15.362 | 7.405 | −29.160 | 1.00 | 30.20 | O |
| ATOM | 2258 | N | LEU | B | 188 | 17.575 | 7.033 | −29.023 | 1.00 | 29.02 | N |
| ATOM | 2259 | CA | LEU | B | 188 | 17.639 | 6.163 | −30.193 | 1.00 | 28.53 | C |
| ATOM | 2260 | CB | LEU | B | 188 | 18.753 | 6.627 | −31.132 | 1.00 | 28.54 | C |
| ATOM | 2261 | CG | LEU | B | 188 | 18.625 | 8.054 | −31.685 | 1.00 | 28.82 | C |
| ATOM | 2262 | CD1 | LEU | B | 188 | 19.932 | 8.505 | −32.322 | 1.00 | 29.02 | C |
| ATOM | 2263 | CD2 | LEU | B | 188 | 17.479 | 8.159 | −32.677 | 1.00 | 28.36 | C |
| ATOM | 2264 | C | LEU | B | 188 | 17.848 | 4.693 | −29.822 | 1.00 | 27.81 | C |
| ATOM | 2265 | O | LEU | B | 188 | 17.204 | 3.819 | −30.390 | 1.00 | 28.69 | O |
| ATOM | 2266 | N | ALA | B | 189 | 18.733 | 4.417 | −28.865 | 1.00 | 27.30 | N |
| ATOM | 2267 | CA | ALA | B | 189 | 19.130 | 3.043 | −28.557 | 1.00 | 26.63 | C |
| ATOM | 2268 | CB | ALA | B | 189 | 20.540 | 2.996 | −27.984 | 1.00 | 26.48 | C |
| ATOM | 2269 | C | ALA | B | 189 | 18.145 | 2.345 | −27.628 | 1.00 | 25.91 | C |
| ATOM | 2270 | O | ALA | B | 189 | 17.680 | 2.931 | −26.660 | 1.00 | 24.79 | O |
| ATOM | 2271 | N | PRO | B | 190 | 17.840 | 1.059 | −27.902 | 1.00 | 26.11 | N |
| ATOM | 2272 | CA | PRO | B | 190 | 17.093 | 0.240 | −26.944 | 1.00 | 25.40 | C |
| ATOM | 2273 | CB | PRO | B | 190 | 16.971 | −1.109 | −27.635 | 1.00 | 25.69 | C |
| ATOM | 2274 | CG | PRO | B | 190 | 17.222 | −0.852 | −29.071 | 1.00 | 26.34 | C |
| ATOM | 2275 | CD | PRO | B | 190 | 18.107 | 0.337 | −29.157 | 1.00 | 26.12 | C |
| ATOM | 2276 | C | PRO | B | 190 | 17.848 | 0.071 | −25.622 | 1.00 | 24.98 | C |
| ATOM | 2277 | O | PRO | B | 190 | 19.073 | −0.019 | −25.637 | 1.00 | 25.38 | O |
| ATOM | 2278 | N | PRO | B | 191 | 17.122 | 0.029 | −24.487 | 1.00 | 24.51 | N |
| ATOM | 2279 | CA | PRO | B | 191 | 17.795 | −0.066 | −23.188 | 1.00 | 24.11 | C |
| ATOM | 2280 | CB | PRO | B | 191 | 16.664 | 0.136 | −22.164 | 1.00 | 24.12 | C |
| ATOM | 2281 | CG | PRO | B | 191 | 15.396 | 0.090 | −22.903 | 1.00 | 24.23 | C |
| ATOM | 2282 | CD | PRO | B | 191 | 15.657 | 0.154 | −24.365 | 1.00 | 23.68 | C |
| ATOM | 2283 | C | PRO | B | 191 | 18.537 | −1.379 | −22.915 | 1.00 | 24.43 | C |
| ATOM | 2284 | O | PRO | B | 191 | 19.439 | −1.377 | −22.072 | 1.00 | 24.90 | O |
| ATOM | 2285 | N | GLN | B | 192 | 18.193 | −2.471 | −23.606 | 1.00 | 23.76 | N |
| ATOM | 2286 | CA | GLN | B | 192 | 18.894 | −3.759 | −23.428 | 1.00 | 23.60 | C |
| ATOM | 2287 | CB | GLN | B | 192 | 17.987 | −4.932 | −23.789 | 1.00 | 25.28 | C |
| ATOM | 2288 | CG | GLN | B | 192 | 16.822 | −5.146 | −22.849 | 1.00 | 27.20 | C |
| ATOM | 2289 | CD | GLN | B | 192 | 16.067 | −6.441 | −23.098 | 1.00 | 29.21 | C |
| ATOM | 2290 | OE1 | GLN | B | 192 | 16.334 | −7.208 | −24.032 | 1.00 | 31.42 | O |
| ATOM | 2291 | NE2 | GLN | B | 192 | 15.092 | −6.686 | −22.253 | 1.00 | 30.20 | N |
| ATOM | 2292 | C | GLN | B | 192 | 20.231 | −3.910 | −24.203 | 1.00 | 22.07 | C |
| ATOM | 2293 | O | GLN | B | 192 | 21.002 | −4.810 | −23.871 | 1.00 | 22.39 | O |
| ATOM | 2294 | N | HIS | B | 193 | 20.468 | −3.074 | −25.219 | 1.00 | 20.20 | N |
| ATOM | 2295 | CA | HIS | B | 193 | 21.610 | −3.269 | −26.136 | 1.00 | 18.99 | C |
| ATOM | 2296 | CB | HIS | B | 193 | 21.429 | −2.507 | −27.467 | 1.00 | 18.43 | C |
| ATOM | 2297 | CG | HIS | B | 193 | 20.436 | −3.133 | −28.396 | 1.00 | 17.95 | C |
| ATOM | 2298 | ND1 | HIS | B | 193 | 20.635 | −3.195 | −29.758 | 1.00 | 17.18 | N |
| ATOM | 2299 | CE1 | HIS | B | 193 | 19.601 | −3.786 | −30.323 | 1.00 | 17.34 | C |
| ATOM | 2300 | NE2 | HIS | B | 193 | 18.751 | −4.132 | −29.374 | 1.00 | 17.56 | N |
| ATOM | 2301 | CD2 | HIS | B | 193 | 19.253 | −3.737 | −28.158 | 1.00 | 17.69 | C |
| ATOM | 2302 | C | HIS | B | 193 | 22.939 | −2.856 | −25.538 | 1.00 | 18.20 | C |
| ATOM | 2303 | O | HIS | B | 193 | 23.061 | −1.736 | −25.025 | 1.00 | 18.22 | O |
| ATOM | 2304 | N | LEU | B | 194 | 23.924 | −3.749 | −25.637 | 1.00 | 17.37 | N |
| ATOM | 2305 | CA | LEU | B | 194 | 25.299 | −3.450 | −25.239 | 1.00 | 17.23 | C |
| ATOM | 2306 | CB | LEU | B | 194 | 26.163 | −4.719 | −25.276 | 1.00 | 17.54 | C |
| ATOM | 2307 | CG | LEU | B | 194 | 27.657 | −4.582 | −24.933 | 1.00 | 18.06 | C |
| ATOM | 2308 | CD1 | LEU | B | 194 | 27.834 | −4.087 | −23.505 | 1.00 | 18.71 | C |
| ATOM | 2309 | CD2 | LEU | B | 194 | 28.407 | −5.864 | −25.195 | 1.00 | 18.41 | C |
| ATOM | 2310 | C | LEU | B | 194 | 25.946 | −2.383 | −26.121 | 1.00 | 16.74 | C |
| ATOM | 2311 | O | LEU | B | 194 | 26.545 | −1.445 | −25.608 | 1.00 | 15.95 | O |
| ATOM | 2312 | N | ILE | B | 195 | 25.829 | −2.537 | −27.444 | 1.00 | 17.14 | N |
| ATOM | 2313 | CA | ILE | B | 195 | 26.573 | −1.700 | −28.388 | 1.00 | 17.15 | C |
| ATOM | 2314 | CB | ILE | B | 195 | 27.184 | −2.514 | −29.549 | 1.00 | 17.12 | C |
| ATOM | 2315 | CG1 | ILE | B | 195 | 27.931 | −3.755 | −29.019 | 1.00 | 17.57 | C |
| ATOM | 2316 | CD1 | ILE | B | 195 | 28.383 | −4.736 | −30.105 | 1.00 | 17.35 | C |
| ATOM | 2317 | CG2 | ILE | B | 195 | 28.128 | −1.625 | −30.356 | 1.00 | 17.14 | C |
| ATOM | 2318 | C | ILE | B | 195 | 25.668 | −0.606 | −28.954 | 1.00 | 17.16 | C |
| ATOM | 2319 | O | ILE | B | 195 | 24.607 | −0.900 | −29.492 | 1.00 | 16.95 | O |
| ATOM | 2320 | N | ARG | B | 196 | 26.108 | 0.646 | −28.823 | 1.00 | 17.85 | N |
| ATOM | 2321 | CA | ARG | B | 196 | 25.487 | 1.786 | −29.480 | 1.00 | 18.60 | C |
| ATOM | 2322 | CB | ARG | B | 196 | 25.098 | 2.855 | −28.470 | 1.00 | 19.18 | C |
| ATOM | 2323 | CG | ARG | B | 196 | 24.113 | 2.397 | −27.420 | 1.00 | 19.84 | C |
| ATOM | 2324 | CD | ARG | B | 196 | 23.819 | 3.530 | −26.452 | 1.00 | 20.28 | C |
| ATOM | 2325 | NE | ARG | B | 196 | 22.885 | 3.126 | −25.398 | 1.00 | 20.99 | N |
| ATOM | 2326 | CZ | ARG | B | 196 | 22.465 | 3.916 | −24.408 | 1.00 | 21.06 | C |
| ATOM | 2327 | NH1 | ARG | B | 196 | 22.880 | 5.181 | −24.304 | 1.00 | 21.03 | N |
| ATOM | 2328 | NH2 | ARG | B | 196 | 21.624 | 3.436 | −23.500 | 1.00 | 21.40 | N |
| ATOM | 2329 | C | ARG | B | 196 | 26.459 | 2.406 | −30.460 | 1.00 | 18.81 | C |
| ATOM | 2330 | O | ARG | B | 196 | 27.661 | 2.167 | −30.389 | 1.00 | 18.09 | O |
| ATOM | 2331 | N | VAL | B | 197 | 25.912 | 3.192 | −31.378 | 1.00 | 19.47 | N |
| ATOM | 2332 | CA | VAL | B | 197 | 26.702 | 4.075 | −32.216 | 1.00 | 20.68 | C |
| ATOM | 2333 | CB | VAL | B | 197 | 26.414 | 3.873 | −33.711 | 1.00 | 20.43 | C |
| ATOM | 2334 | CG1 | VAL | B | 197 | 27.111 | 4.941 | −34.554 | 1.00 | 20.49 | C |
| ATOM | 2335 | CG2 | VAL | B | 197 | 26.841 | 2.478 | −34.133 | 1.00 | 20.28 | C |
| ATOM | 2336 | C | VAL | B | 197 | 26.393 | 5.512 | −31.803 | 1.00 | 21.81 | C |
| ATOM | 2337 | O | VAL | B | 197 | 25.238 | 5.883 | −31.602 | 1.00 | 19.84 | O |
| ATOM | 2338 | N | GLU | B | 198 | 27.456 | 6.300 | −31.696 | 1.00 | 24.80 | N |
| ATOM | 2339 | CA | GLU | B | 198 | 27.403 | 7.687 | −31.266 | 1.00 | 27.11 | C |
| ATOM | 2340 | CB | GLU | B | 198 | 28.572 | 7.926 | −30.314 | 1.00 | 28.69 | C |
| ATOM | 2341 | CG | GLU | B | 198 | 28.603 | 9.262 | −29.597 | 1.00 | 30.73 | C |
| ATOM | 2342 | CD | GLU | B | 198 | 29.958 | 9.541 | −28.956 | 1.00 | 32.49 | C |
| ATOM | 2343 | OE1 | GLU | B | 198 | 30.699 | 8.585 | −28.644 | 1.00 | 30.91 | O |
| ATOM | 2344 | OE2 | GLU | B | 198 | 30.298 | 10.769 | −28.824 | 1.00 | 37.25 | O |
| ATOM | 2345 | C | GLU | B | 198 | 27.539 | 8.589 | −32.492 | 1.00 | 28.40 | C |
| ATOM | 2346 | O | GLU | B | 198 | 28.180 | 8.212 | −33.480 | 1.00 | 30.21 | O |
| ATOM | 2347 | N | GLY | B | 199 | 26.939 | 9.775 | −32.432 | 1.00 | 29.48 | N |
| ATOM | 2348 | CA | GLY | B | 199 | 27.079 | 10.793 | −33.483 | 1.00 | 29.68 | C |
| ATOM | 2349 | C | GLY | B | 199 | 26.660 | 10.381 | −34.881 | 1.00 | 29.89 | C |
| ATOM | 2350 | O | GLY | B | 199 | 27.299 | 10.776 | −35.856 | 1.00 | 28.78 | O |
| ATOM | 2351 | N | ASN | B | 200 | 25.600 | 9.579 | −34.984 | 1.00 | 31.04 | N |
| ATOM | 2352 | CA | ASN | B | 200 | 25.057 | 9.166 | −36.288 | 1.00 | 31.63 | C |
| ATOM | 2353 | CB | ASN | B | 200 | 25.698 | 7.850 | −36.774 | 1.00 | 30.89 | C |
| ATOM | 2354 | CG | ASN | B | 200 | 25.425 | 7.579 | −38.257 | 1.00 | 30.43 | C |
| ATOM | 2355 | OD1 | ASN | B | 200 | 24.334 | 7.842 | −38.758 | 1.00 | 28.75 | O |
| ATOM | 2356 | ND2 | ASN | B | 200 | 26.423 | 7.052 | −38.966 | 1.00 | 27.66 | N |
| ATOM | 2357 | C | ASN | B | 200 | 23.534 | 9.051 | −36.200 | 1.00 | 31.85 | C |
| ATOM | 2358 | O | ASN | B | 200 | 23.002 | 8.083 | −35.656 | 1.00 | 31.93 | O |
| ATOM | 2359 | N | LEU | B | 201 | 22.848 | 10.041 | −36.763 | 1.00 | 32.12 | N |
| ATOM | 2360 | CA | LEU | B | 201 | 21.386 | 10.137 | −36.691 | 1.00 | 31.61 | C |
| ATOM | 2361 | CO | LEU | B | 201 | 20.930 | 11.558 | −37.050 | 1.00 | 32.24 | C |
| ATOM | 2362 | CG | LEU | B | 201 | 21.425 | 12.661 | −36.104 | 1.00 | 33.67 | C |
| ATOM | 2363 | CD1 | LEU | B | 201 | 20.989 | 14.037 | −36.597 | 1.00 | 34.60 | C |
| ATOM | 2364 | CD2 | LEU | B | 201 | 20.940 | 12.430 | −34.676 | 1.00 | 33.66 | C |
| ATOM | 2365 | C | LEU | B | 201 | 20.646 | 9.106 | −37.553 | 1.00 | 29.70 | C |
| ATOM | 2366 | O | LEU | B | 201 | 19.455 | 8.885 | −37.343 | 1.00 | 30.33 | O |
| ATOM | 2367 | N | ARG | B | 202 | 21.345 | 8.476 | −38.503 | 1.00 | 27.99 | N |
| ATOM | 2368 | CA | ARG | B | 202 | 20.779 | 7.373 | −39.304 | 1.00 | 26.89 | C |
| ATOM | 2369 | CB | ARG | B | 202 | 21.379 | 7.416 | −40.717 | 1.00 | 28.21 | C |
| ATOM | 2370 | CG | ARG | B | 202 | 20.978 | 8.658 | −41.500 | 1.00 | 30.47 | C |
| ATOM | 2371 | CD | ARG | B | 202 | 22.024 | 9.051 | −42.520 | 1.00 | 32.67 | C |
| ATOM | 2372 | NE | ARG | B | 202 | 21.879 | 8.399 | −43.828 | 1.00 | 34.03 | N |
| ATOM | 2373 | CZ | ARG | B | 202 | 22.850 | 8.291 | −44.741 | 1.00 | 35.55 | C |
| ATOM | 2374 | NH1 | ARG | B | 202 | 24.096 | 8.723 | −44.503 | 1.00 | 35.94 | N |
| ATOM | 2375 | NH2 | ARG | B | 202 | 22.584 | 7.716 | −45.908 | 1.00 | 36.79 | N |
| ATOM | 2376 | C | ARG | B | 202 | 20.962 | 5.972 | −38.692 | 1.00 | 24.04 | C |
| ATOM | 2377 | O | ARG | B | 202 | 20.747 | 4.978 | −39.371 | 1.00 | 23.56 | O |
| ATOM | 2378 | N | VAL | B | 203 | 21.345 | 5.903 | −37.421 | 1.00 | 22.62 | N |
| ATOM | 2379 | CA | VAL | B | 203 | 21.536 | 4.625 | −36.711 | 1.00 | 21.38 | C |
| ATOM | 2380 | CB | VAL | B | 203 | 22.150 | 4.857 | −35.302 | 1.00 | 21.58 | C |
| ATOM | 2381 | CG1 | VAL | B | 203 | 21.154 | 5.508 | −34.346 | 1.00 | 21.68 | C |
| ATOM | 2382 | CG2 | VAL | B | 203 | 22.690 | 3.559 | −34.715 | 1.00 | 22.30 | C |
| ATOM | 2383 | C | VAL | B | 203 | 20.225 | 3.827 | −36.600 | 1.00 | 20.58 | C |
| ATOM | 2384 | O | VAL | B | 203 | 19.144 | 4.401 | −36.427 | 1.00 | 19.59 | O |
| ATOM | 2385 | N | GLU | B | 204 | 20.340 | 2.504 | −36.708 | 1.00 | 20.14 | N |
| ATOM | 2386 | CA | GLU | B | 204 | 19.191 | 1.597 | −36.652 | 1.00 | 18.91 | C |
| ATOM | 2387 | CB | GLU | B | 204 | 18.833 | 1.129 | −38.069 | 1.00 | 19.54 | C |
| ATOM | 2388 | CG | GLU | B | 204 | 17.910 | −0.085 | −38.175 | 1.00 | 19.75 | C |
| ATOM | 2389 | CD | GLU | B | 204 | 17.730 | −0.572 | −39.601 | 1.00 | 20.02 | C |
| ATOM | 2390 | OE1 | GLU | B | 204 | 17.642 | 0.288 | −40.512 | 1.00 | 19.33 | O |
| ATOM | 2391 | OE2 | GLU | B | 204 | 17.692 | −1.817 | −39.818 | 1.00 | 19.79 | O |
| ATOM | 2392 | C | GLU | B | 204 | 19.546 | 0.416 | −35.772 | 1.00 | 18.47 | C |
| ATOM | 2393 | O | GLU | B | 204 | 20.631 | −0.151 | −35.900 | 1.00 | 17.83 | O |
| ATOM | 2394 | N | TYR | B | 205 | 18.616 | 0.035 | −34.895 | 1.00 | 18.34 | N |
| ATOM | 2395 | CA | TYR | B | 205 | 18.788 | −1.103 | −33.995 | 1.00 | 18.04 | C |
| ATOM | 2396 | CB | TYR | B | 205 | 18.590 | −0.652 | −32.559 | 1.00 | 17.39 | C |
| ATOM | 2397 | CG | TYR | B | 205 | 19.666 | 0.306 | −32.093 | 1.00 | 16.46 | C |
| ATOM | 2398 | CD1 | TYR | B | 205 | 19.541 | 1.677 | −32.317 | 1.00 | 15.50 | C |
| ATOM | 2399 | CE1 | TYR | B | 205 | 20.535 | 2.556 | −31.929 | 1.00 | 15.19 | C |
| ATOM | 2400 | CZ | TYR | B | 205 | 21.661 | 2.075 | −31.286 | 1.00 | 15.29 | C |
| ATOM | 2401 | OH | TYR | B | 205 | 22.643 | 2.944 | −30.864 | 1.00 | 15.37 | O |
| ATOM | 2402 | CE2 | TYR | B | 205 | 21.788 | 0.730 | −31.014 | 1.00 | 15.15 | C |
| ATOM | 2403 | CD2 | TYR | B | 205 | 20.820 | −0.155 | −31.455 | 1.00 | 15.53 | C |
| ATOM | 2404 | C | TYR | B | 205 | 17.832 | −2.240 | −34.329 | 1.00 | 19.03 | C |
| ATOM | 2405 | O | TYR | B | 205 | 16.698 | −2.003 | −34.762 | 1.00 | 19.53 | O |
| ATOM | 2406 | N | LEU | B | 206 | 18.299 | −3.471 | −34.108 | 1.00 | 20.03 | N |
| ATOM | 2407 | CA | LEU | B | 206 | 17.540 | −4.685 | −34.409 | 1.00 | 20.69 | C |
| ATOM | 2408 | CB | LEU | B | 206 | 18.117 | −5.382 | −35.646 | 1.00 | 20.33 | C |
| ATOM | 2409 | CG | LEU | B | 206 | 17.476 | −6.684 | −36.165 | 1.00 | 20.00 | C |
| ATOM | 2410 | CD1 | LEU | B | 206 | 16.092 | −6.429 | −36.743 | 1.00 | 20.11 | C |
| ATOM | 2411 | CD2 | LEU | B | 206 | 18.361 | −7.352 | −37.210 | 1.00 | 19.26 | C |
| ATOM | 2412 | C | LEU | B | 206 | 17.577 | −5.645 | −33.221 | 1.00 | 21.75 | C |
| ATOM | 2413 | O | LEU | B | 206 | 18.644 | −5.934 | −32.669 | 1.00 | 21.99 | O |
| ATOM | 2414 | N | ASP | B | 207 | 16.396 | −6.095 | −32.816 | 1.00 | 22.65 | N |
| ATOM | 2415 | CA | ASP | B | 207 | 16.248 | −7.323 | −32.061 | 1.00 | 24.24 | C |
| ATOM | 2416 | CB | ASP | B | 207 | 15.160 | −7.209 | −30.991 | 1.00 | 24.96 | C |
| ATOM | 2417 | CG | ASP | B | 207 | 15.494 | −6.242 | −29.884 | 1.00 | 25.14 | C |
| ATOM | 2418 | OD1 | ASP | B | 207 | 16.655 | −5.812 | −29.774 | 1.00 | 26.18 | O |
| ATOM | 2419 | OD2 | ASP | B | 207 | 14.565 | −5.912 | −29.115 | 1.00 | 26.87 | O |
| ATOM | 2420 | C | ASP | B | 207 | 15.826 | −8.342 | −33.091 | 1.00 | 24.48 | C |
| ATOM | 2421 | O | ASP | B | 207 | 14.754 | −8.213 | −33.680 | 1.00 | 25.04 | O |
| ATOM | 2422 | N | ASP | B | 208 | 16.670 | −9.341 | −33.339 | 1.00 | 26.04 | N |
| ATOM | 2423 | CA | ASP | B | 208 | 16.348 | −10.352 | −34.334 | 1.00 | 27.24 | C |
| ATOM | 2424 | CB | ASP | B | 208 | 17.551 | −11.231 | −34.624 | 1.00 | 28.06 | C |
| ATOM | 2425 | CG | ASP | B | 208 | 17.354 | −12.097 | −35.873 | 1.00 | 27.79 | C |
| ATOM | 2426 | OD1 | ASP | B | 208 | 16.528 | −13.032 | −35.831 | 1.00 | 28.22 | O |
| ATOM | 2427 | OD2 | ASP | B | 208 | 18.031 | −11.839 | −36.893 | 1.00 | 28.53 | O |
| ATOM | 2428 | C | ASP | B | 208 | 15.162 | −11.188 | −33.839 | 1.00 | 27.82 | C |
| ATOM | 2429 | O | ASP | B | 208 | 15.180 | −11.671 | −32.717 | 1.00 | 26.96 | O |
| ATOM | 2430 | N | ARG | B | 209 | 14.136 | −11.329 | −34.682 | 1.00 | 29.76 | N |
| ATOM | 2431 | CA | ARG | B | 209 | 12.895 | −12.014 | −34.299 | 1.00 | 32.73 | C |
| ATOM | 2432 | CB | ARG | B | 209 | 11.760 | −11.697 | −35.261 | 1.00 | 36.12 | C |
| ATOM | 2433 | CG | ARG | B | 209 | 11.906 | −12.327 | −36.643 | 1.00 | 40.39 | C |
| ATOM | 2434 | CD | ARG | B | 209 | 10.860 | −11.771 | −37.570 | 1.00 | 43.96 | C |
| ATOM | 2435 | NE | ARG | B | 209 | 10.962 | −12.345 | −38.930 | 1.00 | 47.26 | N |
| ATOM | 2436 | CZ | ARG | B | 209 | 11.838 | −11.969 | −39.871 | 1.00 | 49.19 | C |
| ATOM | 2437 | NH1 | ARG | B | 209 | 12.726 | −10.994 | −39.654 | 1.00 | 48.93 | N |
| ATOM | 2438 | NH2 | ARG | B | 209 | 11.823 | −12.579 | −41.053 | 1.00 | 49.94 | N |
| ATOM | 2439 | C | ARG | B | 209 | 13.045 | −13.539 | −34.099 | 1.00 | 31.10 | C |
| ATOM | 2440 | O | ARG | B | 209 | 12.249 | −14.137 | −33.375 | 1.00 | 31.38 | O |
| ATOM | 2441 | N | ASN | B | 209 | 14.051 | −14.150 | −34.734 | 1.00 | 30.11 | N |
| ATOM | 2442 | CA | ASN | B | 210 | 14.300 | −15.596 | −34.601 | 1.00 | 29.95 | C |
| ATOM | 2443 | CB | ASN | B | 210 | 14.657 | −16.196 | −35.962 | 1.00 | 30.03 | C |
| ATOM | 2444 | CG | ASN | B | 210 | 13.570 | −15.958 | −37.011 | 1.00 | 30.82 | C |
| ATOM | 2445 | OD1 | ASN | B | 210 | 13.868 | −15.746 | −38.189 | 1.00 | 32.69 | O |
| ATOM | 2446 | ND2 | ASN | B | 210 | 12.314 | −15.965 | −36.587 | 1.00 | 30.30 | N |
| ATOM | 2447 | C | ASN | B | 210 | 15.386 | −15.944 | −33.580 | 1.00 | 29.40 | C |
| ATOM | 2448 | O | ASN | B | 210 | 15.193 | −16.857 | −32.778 | 1.00 | 31.13 | O |
| ATOM | 2449 | N | THR | B | 211 | 16.511 | −15.223 | −33.604 | 1.00 | 27.51 | N |
| ATOM | 2450 | CA | THR | B | 211 | 17.650 | −15.491 | −32.706 | 1.00 | 26.85 | C |
| ATOM | 2451 | CB | THR | B | 211 | 18.999 | −15.235 | −33.408 | 1.00 | 26.81 | C |
| ATOM | 2452 | OG1 | THR | B | 211 | 19.128 | −13.849 | −33.737 | 1.00 | 25.92 | O |
| ATOM | 2453 | CG2 | THR | B | 211 | 19.127 | −16.078 | −34.680 | 1.00 | 26.48 | C |
| ATOM | 2454 | C | THR | B | 211 | 17.655 | −14.659 | −31.420 | 1.00 | 26.19 | C |
| ATOM | 2455 | O | THR | B | 211 | 18.382 | −14.985 | −30.482 | 1.00 | 25.69 | O |
| ATOM | 2456 | N | PHE | B | 212 | 16.873 | −13.575 | −31.398 | 1.00 | 26.76 | N |
| ATOM | 2457 | CA | PHE | B | 212 | 16.808 | −12.619 | −30.272 | 1.00 | 25.84 | C |
| ATOM | 2458 | CB | PHE | B | 212 | 16.360 | −13.298 | −28.964 | 1.00 | 27.55 | C |
| ATOM | 2459 | CG | PHE | B | 212 | 15.180 | −14.213 | −29.124 | 1.00 | 29.26 | C |
| ATOM | 2460 | CD1 | PHE | B | 212 | 13.939 | −13.712 | −29.494 | 1.00 | 29.92 | C |
| ATOM | 2461 | CE1 | PHE | B | 212 | 12.843 | −14.558 | −29.614 | 1.00 | 30.92 | C |
| ATOM | 2462 | CZ | PHE | B | 212 | 12.991 | −15.924 | −29.405 | 1.00 | 31.92 | C |
| ATOM | 2463 | CE2 | PHE | B | 212 | 14.232 | −16.442 | −29.057 | 1.00 | 31.63 | C |
| ATOM | 2464 | CD2 | PHE | B | 212 | 15.317 | −15.588 | −28.914 | 1.00 | 31.16 | C |
| ATOM | 2465 | C | PHE | B | 212 | 18.092 | −11.822 | −30.043 | 1.00 | 24.05 | C |
| ATOM | 2466 | O | PHE | B | 212 | 18.181 | −11.105 | −29.050 | 1.00 | 23.28 | O |
| ATOM | 2467 | N | ARG | B | 213 | 19.043 | −11.887 | −30.984 | 1.00 | 22.90 | N |
| ATOM | 2468 | CA | ARG | B | 213 | 20.326 | −11.215 | −30.803 | 1.00 | 21.49 | C |
| ATOM | 2469 | CB | ARG | B | 213 | 21.430 | −11.919 | −31.585 | 1.00 | 21.91 | C |
| ATOM | 2470 | CG | ARG | B | 213 | 21.793 | −13.267 | −30.988 | 1.00 | 22.84 | C |
| ATOM | 2471 | CD | ARG | B | 213 | 22.968 | −13.913 | −31.661 | 1.00 | 23.46 | C |
| ATOM | 2472 | NE | ARG | B | 213 | 23.299 | −15.239 | −31.126 | 1.00 | 24.14 | N |
| ATOM | 2473 | CZ | ARG | B | 213 | 24.059 | −15.489 | −30.050 | 1.00 | 24.24 | C |
| ATOM | 2474 | NH1 | ARG | B | 213 | 24.596 | −14.504 | −29.338 | 1.00 | 23.38 | N |
| ATOM | 2475 | NH2 | ARG | B | 213 | 24.301 | −16.751 | −29.680 | 1.00 | 24.82 | N |
| ATOM | 2476 | C | ARG | B | 213 | 20.205 | −9.742 | −31.206 | 1.00 | 20.60 | C |
| ATOM | 2477 | O | ARG | B | 213 | 19.367 | −9.369 | −32.058 | 1.00 | 19.33 | O |
| ATOM | 2478 | N | HIS | B | 214 | 21.035 | −8.921 | −30.565 | 1.00 | 19.72 | N |
| ATOM | 2479 | CA | HIS | B | 214 | 20.976 | −7.474 | −30.714 | 1.00 | 19.19 | C |
| ATOM | 2480 | CB | HIS | B | 214 | 21.187 | −6.790 | −29.363 | 1.00 | 19.02 | C |
| ATOM | 2481 | CG | HIS | B | 214 | 20.153 | −7.141 | −28.342 | 1.00 | 19.28 | C |
| ATOM | 2482 | ND1 | HIS | B | 214 | 20.367 | −7.015 | −26.985 | 1.00 | 19.65 | N |
| ATOM | 2483 | CE1 | HIS | B | 214 | 19.285 | −7.399 | −26.331 | 1.00 | 19.16 | C |
| ATOM | 2484 | NE2 | HIS | B | 214 | 18.376 | −7.766 | −27.213 | 1.00 | 19.07 | N |
| ATOM | 2485 | CD2 | HIS | B | 214 | 18.895 | −7.621 | −28.478 | 1.00 | 19.73 | C |
| ATOM | 2486 | C | HIS | B | 214 | 22.050 | −7.024 | −31.680 | 1.00 | 18.37 | C |
| ATOM | 2487 | O | HIS | B | 214 | 23.168 | −7.564 | −31.675 | 1.00 | 18.45 | O |
| ATOM | 2488 | N | SER | B | 215 | 21.724 | −6.050 | −32.518 | 1.00 | 16.91 | N |
| ATOM | 2489 | CA | SER | B | 215 | 22.732 | −5.442 | −33.375 | 1.00 | 16.34 | C |
| ATOM | 2490 | CB | SER | B | 215 | 22.882 | −6.240 | −34.673 | 1.00 | 16.02 | C |
| ATOM | 2491 | OG | SER | B | 215 | 21.683 | −6.251 | −35.427 | 1.00 | 16.01 | O |
| ATOM | 2492 | C | SER | B | 215 | 22.401 | −3.983 | −33.667 | 1.00 | 15.90 | C |
| ATOM | 2493 | O | SER | B | 215 | 21.269 | −3.535 | −33.469 | 1.00 | 15.16 | O |
| ATOM | 2494 | N | VAL | B | 216 | 23.408 | −3.259 | −34.140 | 1.00 | 15.65 | N |
| ATOM | 2495 | CA | VAL | B | 216 | 23.273 | −1.854 | −34.497 | 1.00 | 16.18 | C |
| ATOM | 2496 | CB | VAL | B | 216 | 23.896 | −0.918 | −33.437 | 1.00 | 16.12 | C |
| ATOM | 2497 | CG1 | VAL | B | 216 | 25.373 | −1.223 | −33.196 | 1.00 | 16.54 | C |
| ATOM | 2498 | CG2 | VAL | B | 216 | 23.712 | 0.546 | −33.819 | 1.00 | 15.93 | C |
| ATOM | 2499 | C | VAL | B | 216 | 23.893 | −1.656 | −35.879 | 1.00 | 16.95 | C |
| ATOM | 2500 | O | VAL | B | 216 | 25.007 | −2.127 | −36.143 | 1.00 | 16.85 | O |
| ATOM | 2501 | N | VAL | B | 216 | 23.160 | −0.976 | −36.758 | 1.00 | 17.44 | N |
| ATOM | 2502 | CA | VAL | B | 217 | 23.579 | −0.818 | −38.142 | 1.00 | 18.66 | C |
| ATOM | 2503 | CB | VAL | B | 217 | 22.756 | −1.736 | −39.091 | 1.00 | 19.09 | C |
| ATOM | 2504 | CG1 | VAL | B | 217 | 21.264 | −1.443 | −39.034 | 1.00 | 19.58 | C |
| ATOM | 2505 | CG2 | VAL | B | 217 | 23.247 | −1.633 | −40.522 | 1.00 | 19.28 | C |
| ATOM | 2506 | C | VAL | B | 217 | 23.543 | 0.663 | −38.546 | 1.00 | 19.47 | C |
| ATOM | 2507 | O | VAL | B | 217 | 22.675 | 1.414 | −38.106 | 1.00 | 19.53 | O |
| ATOM | 2508 | N | VAL | B | 218 | 24.532 | 1.076 | −39.342 | 1.00 | 20.84 | N |
| ATOM | 2509 | CA | VAL | B | 218 | 24.579 | 2.425 | −39.926 | 1.00 | 22.19 | C |
| ATOM | 2510 | CB | VAL | B | 218 | 25.630 | 3.350 | −39.256 | 1.00 | 21.64 | C |
| ATOM | 2511 | CG1 | VAL | B | 218 | 25.215 | 3.707 | −37.843 | 1.00 | 21.71 | C |
| ATOM | 2512 | CG2 | VAL | B | 218 | 27.028 | 2.732 | −39.266 | 1.00 | 21.96 | C |
| ATOM | 2513 | C | VAL | B | 218 | 24.885 | 2.311 | −41.413 | 1.00 | 23.23 | C |
| ATOM | 2514 | O | VAL | B | 218 | 25.414 | 1.283 | −41.856 | 1.00 | 24.19 | O |
| ATOM | 2515 | N | PRO | B | 219 | 24.552 | 3.358 | −42.195 | 1.00 | 24.86 | N |
| ATOM | 2516 | CA | PRO | B | 219 | 24.970 | 3.346 | −43.600 | 1.00 | 25.77 | C |
| ATOM | 2517 | CB | PRO | B | 219 | 24.315 | 4.608 | −44.178 | 1.00 | 25.18 | C |
| ATOM | 2518 | CG | PRO | B | 219 | 23.174 | 4.901 | −43.274 | 1.00 | 25.28 | C |
| ATOM | 2519 | CD | PRO | B | 219 | 23.635 | 4.482 | −41.910 | 1.00 | 25.73 | C |
| ATOM | 2520 | C | PRO | B | 219 | 26.493 | 3.441 | −43.717 | 1.00 | 26.69 | C |
| ATOM | 2521 | O | PRO | B | 219 | 27.125 | 4.233 | −42.993 | 1.00 | 27.84 | O |
| ATOM | 2522 | N | TYR | B | 220 | 27.069 | 2.630 | −44.596 | 1.00 | 27.08 | N |
| ATOM | 2523 | CA | TYR | B | 220 | 26.459 | 2.786 | −44.970 | 1.00 | 28.90 | C |
| ATOM | 2524 | CB | TYR | B | 220 | 28.946 | 1.621 | −45.828 | 1.00 | 28.39 | C |
| ATOM | 2525 | CG | TYR | B | 220 | 30.353 | 1.826 | −46.339 | 1.00 | 28.98 | C |
| ATOM | 2526 | CD1 | TYR | B | 220 | 31.464 | 1.562 | −45.521 | 1.00 | 29.08 | C |
| ATOM | 2527 | CE1 | TYR | B | 220 | 32.755 | 1.768 | −45.973 | 1.00 | 29.24 | C |
| ATOM | 2528 | CZ | TYR | B | 220 | 32.954 | 2.243 | −47.259 | 1.00 | 29.80 | C |
| ATOM | 2529 | OH | TYR | B | 220 | 34.226 | 2.438 | −47.721 | 1.00 | 30.13 | O |
| ATOM | 2530 | CE2 | TYR | B | 220 | 31.670 | 2.524 | −48.066 | 1.00 | 29.19 | C |
| ATOM | 2531 | CD2 | TYR | B | 220 | 30.583 | 2.313 | −47.623 | 1.00 | 28.57 | C |
| ATOM | 2532 | C | TYR | B | 220 | 28.611 | 4.084 | −45.752 | 1.00 | 30.25 | C |
| ATOM | 2533 | O | TYR | B | 220 | 27.972 | 4.264 | −46.788 | 1.00 | 32.27 | O |
| ATOM | 2534 | N | GLU | B | 221 | 29.443 | 4.983 | −45.237 | 1.00 | 33.03 | N |
| ATOM | 2535 | CA | GLU | B | 221 | 29.873 | 6.161 | −45.973 | 1.00 | 36.06 | C |
| ATOM | 2536 | CB | GLU | B | 221 | 29.711 | 7.430 | −45.133 | 1.00 | 37.65 | C |
| ATOM | 2537 | CG | GLU | B | 221 | 28.257 | 7.876 | −44.948 | 1.00 | 39.66 | C |
| ATOM | 2538 | CD | GLU | B | 221 | 27.580 | 8.321 | −46.247 | 1.00 | 41.65 | C |
| ATOM | 2539 | OE1 | GLU | B | 221 | 28.290 | 8.745 | −47.194 | 1.00 | 41.99 | O |
| ATOM | 2540 | OE2 | GLU | B | 221 | 26.326 | 8.273 | −46.310 | 1.00 | 42.48 | O |
| ATOM | 2541 | C | GLU | B | 221 | 31.332 | 5.941 | −46.361 | 1.00 | 36.29 | C |
| ATOM | 2542 | O | GLU | B | 221 | 32.101 | 5.450 | −45.545 | 1.00 | 33.61 | O |
| ATOM | 2543 | N | PRO | B | 222 | 31.705 | 6.276 | −47.616 | 1.00 | 38.15 | N |
| ATOM | 2544 | CA | PRO | B | 222 | 33.127 | 6.131 | −47.963 | 1.00 | 38.79 | C |
| ATOM | 2545 | CB | PRO | B | 222 | 33.150 | 6.336 | −49.485 | 1.00 | 39.12 | C |
| ATOM | 2546 | CG | PRO | B | 222 | 31.867 | 7.024 | −49.817 | 1.00 | 39.12 | C |
| ATOM | 2547 | CD | PRO | B | 222 | 30.868 | 6.617 | −48.784 | 1.00 | 38.33 | C |
| ATOM | 2548 | C | PRO | B | 222 | 34.004 | 7.146 | −47.233 | 1.00 | 39.58 | C |
| ATOM | 2549 | O | PRO | B | 222 | 33.482 | 8.137 | −46.710 | 1.00 | 39.36 | O |
| ATOM | 2550 | N | PRO | B | 223 | 35.336 | 6.907 | −47.181 | 1.00 | 41.00 | N |
| ATOM | 2551 | CA | PRO | B | 223 | 36.251 | 7.800 | −46.457 | 1.00 | 42.63 | C |
| ATOM | 2552 | CB | PRO | B | 223 | 37.629 | 7.279 | −46.850 | 1.00 | 41.94 | C |
| ATOM | 2553 | CG | PRO | B | 223 | 37.430 | 5.872 | −47.228 | 1.00 | 41.12 | C |
| ATOM | 2554 | CD | PRO | B | 223 | 36.039 | 5.749 | −47.759 | 1.00 | 40.40 | C |
| ATOM | 2555 | C | PRO | B | 223 | 36.108 | 9.277 | −46.877 | 1.00 | 45.39 | C |
| ATOM | 2556 | O | PRO | B | 223 | 35.843 | 9.577 | −48.054 | 1.00 | 42.21 | O |
| ATOM | 2557 | N | GLU | B | 224 | 36.277 | 10.183 | −45.913 | 1.00 | 50.30 | N |
| ATOM | 2558 | CA | GLU | B | 224 | 36.271 | 11.614 | −46.203 | 1.00 | 55.58 | C |
| ATOM | 2559 | CB | GLU | B | 224 | 36.197 | 12.469 | −44.928 | 1.00 | 58.43 | C |
| ATOM | 2560 | CG | GLU | B | 224 | 34.958 | 12.258 | −44.063 | 1.00 | 61.20 | C |
| ATOM | 2561 | CD | GLU | B | 224 | 33.664 | 12.787 | −44.662 | 1.00 | 63.10 | C |
| ATOM | 2562 | OE1 | GLU | B | 224 | 32.670 | 12.796 | −43.912 | 1.00 | 64.19 | O |
| ATOM | 2563 | OE2 | GLU | B | 224 | 33.600 | 13.192 | −45.843 | 1.00 | 64.31 | O |
| ATOM | 2564 | C | GLU | B | 224 | 37.522 | 11.976 | −47.002 | 1.00 | 58.30 | C |
| ATOM | 2565 | O | GLU | B | 224 | 38.444 | 11.173 | −47.130 | 1.00 | 58.62 | O |
| ATOM | 2566 | N | VAL | B | 225 | 37.517 | 13.180 | −47.565 | 1.00 | 62.43 | N |
| ATOM | 2567 | CA | VAL | B | 225 | 38.559 | 13.591 | −48.502 | 1.00 | 65.34 | C |
| ATOM | 2568 | CB | VAL | B | 225 | 38.187 | 14.905 | −49.242 | 1.00 | 66.89 | C |
| ATOM | 2569 | CG1 | VAL | B | 225 | 39.288 | 15.311 | −50.191 | 1.00 | 68.53 | C |
| ATOM | 2570 | CG2 | VAL | B | 225 | 36.887 | 14.737 | −50.027 | 1.00 | 67.44 | C |
| ATOM | 2571 | C | VAL | B | 225 | 39.873 | 13.709 | −47.712 | 1.00 | 66.11 | C |
| ATOM | 2572 | O | VAL | B | 225 | 39.917 | 14.349 | −46.653 | 1.00 | 62.59 | O |
| ATOM | 2573 | N | GLY | B | 226 | 40.916 | 13.045 | −48.214 | 1.00 | 65.76 | N |
| ATOM | 2574 | CA | GLY | B | 226 | 42.199 | 12.943 | −47.511 | 1.00 | 65.29 | C |
| ATOM | 2575 | C | GLY | B | 226 | 42.440 | 11.638 | −46.764 | 1.00 | 64.55 | C |
| ATOM | 2576 | O | GLY | B | 226 | 43.607 | 11.248 | −46.582 | 1.00 | 66.24 | O |
| ATOM | 2577 | N | SER | B | 227 | 41.361 | 10.951 | −46.362 | 1.00 | 60.40 | N |
| ATOM | 2578 | CA | SER | B | 227 | 41.457 | 9.744 | −45.535 | 1.00 | 56.66 | C |
| ATOM | 2579 | CB | SER | B | 227 | 40.349 | 9.721 | −44.476 | 1.00 | 59.08 | C |
| ATOM | 2580 | OG | SER | B | 227 | 40.320 | 10.929 | −43.737 | 1.00 | 61.52 | O |
| ATOM | 2581 | C | SER | B | 227 | 41.359 | 8.467 | −46.367 | 1.00 | 51.19 | C |
| ATOM | 2582 | O | SER | B | 227 | 40.852 | 8.481 | −47.480 | 1.00 | 47.02 | O |
| ATOM | 2583 | N | ASP | B | 228 | 41.920 | 7.381 | −45.824 | 1.00 | 47.41 | N |
| ATOM | 2584 | CA | ASP | B | 228 | 41.852 | 6.059 | −46.429 | 1.00 | 46.65 | C |
| ATOM | 2585 | CB | ASP | B | 228 | 43.253 | 5.442 | −46.492 | 1.00 | 49.03 | C |
| ATOM | 2586 | CG | ASP | B | 228 | 44.124 | 6.074 | −47.575 | 1.00 | 51.48 | C |
| ATOM | 2587 | OD1 | ASP | B | 228 | 43.959 | 7.257 | −47.903 | 1.00 | 52.28 | O |
| ATOM | 2588 | OD2 | ASP | B | 228 | 45.014 | 5.357 | −48.122 | 1.00 | 53.12 | O |
| ATOM | 2589 | C | ASP | B | 228 | 40.891 | 5.112 | −45.679 | 1.00 | 42.17 | C |
| ATOM | 2590 | O | ASP | B | 228 | 40.789 | 3.936 | −46.080 | 1.00 | 42.50 | O |
| ATOM | 2591 | N | CYS | B | 229 | 40.176 | 5.621 | −44.660 | 1.00 | 37.20 | N |
| ATOM | 2592 | CA | CYS | B | 229 | 39.233 | 4.791 | −43.923 | 1.00 | 33.53 | C |
| ATOM | 2593 | CB | CYS | B | 229 | 39.929 | 4.105 | −42.741 | 1.00 | 33.14 | C |
| ATOM | 2594 | SG | CYS | B | 229 | 40.536 | 5.214 | −41.473 | 1.00 | 32.49 | S |
| ATOM | 2595 | C | CYS | B | 229 | 38.014 | 5.546 | −43.425 | 1.00 | 30.86 | C |
| ATOM | 2596 | O | CYS | B | 229 | 38.026 | 6.759 | −43.364 | 1.00 | 29.78 | O |
| ATOM | 2597 | N | THR | B | 230 | 36.974 | 4.790 | −43.088 | 1.00 | 29.68 | N |
| ATOM | 2598 | CA | THR | B | 230 | 35.757 | 5.318 | −42.470 | 1.00 | 28.08 | C |
| ATOM | 2599 | CB | THR | B | 230 | 34.502 | 4.700 | −43.112 | 1.00 | 28.58 | C |
| ATOM | 2600 | OG1 | THR | B | 230 | 34.551 | 4.865 | −44.539 | 1.00 | 29.60 | O |
| ATOM | 2601 | CG2 | THR | B | 230 | 33.236 | 5.346 | −42.544 | 1.00 | 28.42 | C |
| ATOM | 2602 | C | THR | B | 230 | 35.761 | 4.952 | −40.993 | 1.00 | 26.64 | C |
| ATOM | 2603 | O | THR | B | 230 | 35.968 | 3.784 | −40.647 | 1.00 | 26.38 | O |
| ATOM | 2604 | N | THR | B | 231 | 35.478 | 5.931 | −40.134 | 1.00 | 25.32 | N |
| ATOM | 2605 | CA | THR | B | 231 | 35.499 | 5.746 | −38.678 | 1.00 | 24.50 | C |
| ATOM | 2606 | CB | THR | B | 231 | 36.376 | 6.828 | −38.013 | 1.00 | 23.56 | C |
| ATOM | 2607 | OG1 | THR | B | 231 | 37.693 | 6.788 | −38.571 | 1.00 | 22.91 | O |
| ATOM | 2608 | CG2 | THR | B | 231 | 36.471 | 6.620 | −36.509 | 1.00 | 23.89 | C |
| ATOM | 2609 | C | THR | B | 231 | 34.086 | 5.807 | −38.086 | 1.00 | 24.56 | C |
| ATOM | 2610 | O | THR | B | 231 | 33.375 | 6.789 | −38.269 | 1.00 | 25.79 | O |
| ATOM | 2611 | N | ILE | B | 232 | 33.698 | 4.749 | −37.378 | 1.00 | 23.95 | N |
| ATOM | 2612 | CA | ILE | B | 232 | 32.445 | 4.700 | −36.618 | 1.00 | 23.24 | C |
| ATOM | 2613 | CB | ILE | B | 232 | 31.663 | 3.402 | −36.901 | 1.00 | 22.90 | C |
| ATOM | 2614 | CG1 | ILE | B | 232 | 31.224 | 3.369 | −38.371 | 1.00 | 23.05 | C |
| ATOM | 2615 | CD1 | ILE | B | 232 | 30.680 | 2.025 | −38.857 | 1.00 | 23.42 | C |
| ATOM | 2616 | CG2 | ILE | B | 232 | 30.450 | 3.281 | −35.994 | 1.00 | 23.27 | C |
| ATOM | 2617 | C | ILE | B | 232 | 32.800 | 4.805 | −35.129 | 1.00 | 23.30 | C |
| ATOM | 2618 | O | ILE | B | 232 | 33.785 | 4.214 | −34.683 | 1.00 | 23.10 | O |
| ATOM | 2619 | N | HIS | B | 233 | 31.991 | 5.547 | −34.374 | 1.00 | 23.28 | N |
| ATOM | 2620 | CA | HIS | B | 233 | 32.172 | 5.686 | −32.930 | 1.00 | 23.73 | C |
| ATOM | 2621 | CB | HIS | B | 233 | 32.022 | 7.145 | −32.478 | 1.00 | 25.19 | C |
| ATOM | 2622 | CG | HIS | B | 233 | 33.163 | 8.026 | −32.884 | 1.00 | 26.52 | C |
| ATOM | 2623 | ND1 | HIS | B | 233 | 33.074 | 9.403 | −32.885 | 1.00 | 28.15 | N |
| ATOM | 2624 | CE1 | HIS | B | 233 | 34.223 | 9.919 | −33.283 | 1.00 | 28.58 | C |
| ATOM | 2625 | NE2 | HIS | B | 233 | 35.056 | 8.927 | −33.546 | 1.00 | 28.26 | N |
| ATOM | 2626 | CD2 | HIS | B | 233 | 34.418 | 7.734 | −33.301 | 1.00 | 27.31 | C |
| ATOM | 2627 | C | HIS | B | 233 | 31.170 | 4.817 | −32.183 | 1.00 | 22.34 | C |
| ATOM | 2628 | O | HIS | B | 233 | 29.994 | 5.165 | −32.093 | 1.00 | 22.17 | O |
| ATOM | 2629 | N | TYR | B | 234 | 31.647 | 3.686 | −31.655 | 1.00 | 21.09 | N |
| ATOM | 2630 | CA | TYR | B | 234 | 30.828 | 2.801 | −30.837 | 1.00 | 20.44 | C |
| ATOM | 2631 | CB | TYR | B | 234 | 31.241 | 1.338 | −31.041 | 1.00 | 19.73 | C |
| ATOM | 2632 | CG | TYR | B | 234 | 30.932 | 0.795 | −32.422 | 1.00 | 19.09 | C |
| ATOM | 2633 | CD1 | TYR | B | 234 | 29.616 | 0.526 | −32.814 | 1.00 | 18.52 | C |
| ATOM | 2634 | CE1 | TYR | B | 234 | 29.327 | 0.021 | −34.079 | 1.00 | 18.13 | C |
| ATOM | 2635 | CZ | TYR | B | 234 | 30.362 | −0.220 | −34.971 | 1.00 | 18.18 | C |
| ATOM | 2636 | OH | TYR | B | 234 | 30.087 | −0.708 | −36.225 | 1.00 | 17.60 | O |
| ATOM | 2637 | CE2 | TYR | B | 234 | 31.672 | 0.027 | −34.605 | 1.00 | 17.91 | C |
| ATOM | 2638 | CD2 | TYR | B | 234 | 31.953 | 0.531 | −33.337 | 1.00 | 18.33 | C |
| ATOM | 2639 | C | TYR | B | 234 | 30.928 | 3.177 | −29.354 | 1.00 | 20.96 | C |
| ATOM | 2640 | O | TYR | B | 234 | 31.925 | 3.762 | −28.917 | 1.00 | 22.04 | O |
| ATOM | 2641 | N | ASN | B | 235 | 29.882 | 2.861 | −28.594 | 1.00 | 20.91 | N |
| ATOM | 2642 | CA | ASN | B | 235 | 29.902 | 2.938 | −27.132 | 1.00 | 20.65 | C |
| ATOM | 2643 | CB | ASN | B | 235 | 28.931 | 3.991 | −26.604 | 1.00 | 21.11 | C |
| ATOM | 2644 | CG | ASN | B | 235 | 29.156 | 5.381 | −27.207 | 1.00 | 22.25 | C |
| ATOM | 2645 | OD1 | ASN | B | 235 | 28.205 | 6.124 | −27.389 | 1.00 | 23.97 | O |
| ATOM | 2646 | ND2 | ASN | B | 235 | 30.402 | 5.743 | −27.511 | 1.00 | 22.21 | N |
| ATOM | 2647 | C | ASN | B | 235 | 29.473 | 1.574 | −26.587 | 1.00 | 20.43 | C |
| ATOM | 2648 | O | ASN | B | 235 | 28.516 | 0.985 | −27.086 | 1.00 | 18.74 | O |
| ATOM | 2649 | N | TYR | B | 236 | 30.173 | 1.088 | −25.564 | 1.00 | 20.57 | N |
| ATOM | 2650 | CA | TYR | B | 236 | 29.797 | −0.138 | −24.865 | 1.00 | 21.24 | C |
| ATOM | 2651 | CB | TYR | B | 236 | 31.001 | −1.054 | −24.735 | 1.00 | 21.10 | C |
| ATOM | 2652 | CG | TYR | B | 236 | 31.483 | −1.599 | −26.068 | 1.00 | 21.16 | C |
| ATOM | 2653 | CD1 | TYR | B | 236 | 30.959 | −2.782 | −26.585 | 1.00 | 20.60 | C |
| ATOM | 2654 | CE1 | TYR | B | 236 | 31.392 | −3.299 | −27.802 | 1.00 | 20.32 | C |
| ATOM | 2655 | CZ | TYR | B | 236 | 32.367 | −2.623 | −28.520 | 1.00 | 20.78 | C |
| ATOM | 2656 | OH | TYR | B | 236 | 32.802 | −3.121 | −29.729 | 1.00 | 20.09 | O |
| ATOM | 2657 | CE2 | TYR | B | 236 | 32.905 | −1.432 | −28.030 | 1.00 | 20.69 | C |
| ATOM | 2658 | CD2 | TYR | B | 236 | 32.457 | −0.929 | −26.818 | 1.00 | 20.91 | C |
| ATOM | 2659 | C | TYR | B | 236 | 29.200 | 0.222 | −23.500 | 1.00 | 21.79 | C |
| ATOM | 2660 | O | TYR | B | 236 | 29.852 | 0.889 | −22.698 | 1.00 | 22.22 | O |
| ATOM | 2661 | N | MET | B | 237 | 27.964 | −0.210 | −23.264 | 1.00 | 22.69 | N |
| ATOM | 2662 | CA | MET | B | 237 | 27.122 | 0.305 | −22.184 | 1.00 | 23.93 | C |
| ATOM | 2663 | CB | MET | B | 237 | 25.754 | 0.708 | −22.784 | 1.00 | 23.80 | C |
| ATOM | 2664 | CG | MET | B | 237 | 25.818 | 1.662 | −23.954 | 1.00 | 24.39 | C |
| ATOM | 2665 | SD | MET | B | 237 | 26.593 | 3.247 | −23.592 | 1.00 | 25.49 | S |
| ATOM | 2666 | CE | MET | B | 237 | 25.680 | 3.829 | −22.138 | 1.00 | 25.44 | C |
| ATOM | 2667 | C | MET | B | 237 | 26.926 | −0.617 | −20.972 | 1.00 | 24.33 | C |
| ATOM | 2668 | O | MET | B | 237 | 26.006 | −0.393 | −20.194 | 1.00 | 22.15 | O |
| ATOM | 2669 | N | CYS | B | 238 | 27.759 | −1.647 | −20.860 | 1.00 | 26.69 | N |
| ATOM | 2670 | CA | CYS | B | 238 | 27.690 | −2.611 | −19.750 | 1.00 | 29.46 | C |
| ATOM | 2671 | CB | CYS | B | 238 | 26.623 | −3.703 | −20.020 | 1.00 | 29.94 | C |
| ATOM | 2672 | SG | CYS | B | 238 | 26.277 | −4.817 | −18.629 | 1.00 | 30.15 | S |
| ATOM | 2673 | C | CYS | B | 238 | 29.087 | −3.251 | −19.643 | 1.00 | 31.46 | C |
| ATOM | 2674 | O | CYS | B | 238 | 29.967 | −3.060 | −20.518 | 1.00 | 30.23 | O |
| ATOM | 2675 | N | ASN | B | 239 | 29.306 | −3.987 | −18.557 | 1.00 | 34.00 | N |
| ATOM | 2676 | CA | ASN | B | 239 | 30.587 | −4.632 | −18.263 | 1.00 | 36.14 | C |
| ATOM | 2677 | CB | ASN | B | 239 | 31.105 | −4.254 | −16.872 | 1.00 | 37.23 | C |
| ATOM | 2678 | CG | ASN | B | 239 | 32.070 | −3.122 | −16.898 | 1.00 | 38.77 | C |
| ATOM | 2679 | OD1 | ASN | B | 239 | 33.223 | −3.304 | −16.518 | 1.00 | 39.30 | O |
| ATOM | 2680 | ND2 | ASN | B | 239 | 31.622 | −1.941 | −17.326 | 1.00 | 39.92 | N |
| ATOM | 2681 | C | ASN | B | 239 | 30.362 | −6.129 | −18.284 | 1.00 | 38.81 | C |
| ATOM | 2682 | O | ASN | B | 239 | 29.278 | −6.612 | −17.914 | 1.00 | 37.41 | O |
| ATOM | 2683 | N | SER | B | 240 | 31.410 | −6.900 | −18.591 | 1.00 | 41.68 | N |
| ATOM | 2684 | CA | SER | B | 240 | 31.439 | −8.329 | −18.224 | 1.00 | 43.84 | C |
| ATOM | 2685 | CB | SER | B | 240 | 32.587 | −9.059 | −18.926 | 1.00 | 44.23 | C |
| ATOM | 2686 | OG | SER | B | 240 | 33.848 | −8.553 | −18.533 | 1.00 | 44.13 | O |
| ATOM | 2687 | C | SER | B | 240 | 31.528 | −8.506 | −16.696 | 1.00 | 44.04 | C |
| ATOM | 2688 | O | SER | B | 240 | 31.000 | −9.482 | −16.166 | 1.00 | 47.07 | O |
| ATOM | 2689 | N | SER | B | 241 | 32.191 | −7.565 | −16.012 | 1.00 | 43.16 | N |
| ATOM | 2690 | CA | SER | B | 241 | 32.306 | −7.584 | −14.554 | 1.00 | 44.61 | C |
| ATOM | 2691 | CB | SER | B | 241 | 33.413 | −6.615 | −14.091 | 1.00 | 45.21 | C |
| ATOM | 2692 | OG | SER | B | 241 | 33.034 | −5.263 | −14.267 | 1.00 | 46.25 | O |
| ATOM | 2693 | C | SER | B | 241 | 30.992 | −7.274 | −13.804 | 1.00 | 44.10 | C |
| ATOM | 2694 | O | SER | B | 241 | 30.906 | −7.492 | −12.598 | 1.00 | 44.77 | O |
| ATOM | 2695 | N | CYS | B | 242 | 29.979 | −6.761 | −14.509 | 1.00 | 42.68 | N |
| ATOM | 2696 | CA | CYS | B | 242 | 28.692 | −6.389 | −13.904 | 1.00 | 40.82 | C |
| ATOM | 2697 | CB | CYS | B | 242 | 27.716 | −5.908 | −14.972 | 1.00 | 37.01 | C |
| ATOM | 2698 | SG | CYS | B | 242 | 27.831 | −4.154 | −15.383 | 1.00 | 33.03 | S |
| ATOM | 2699 | C | CYS | B | 242 | 28.048 | −7.514 | −13.095 | 1.00 | 43.09 | C |
| ATOM | 2700 | O | CYS | B | 242 | 27.853 | −8.622 | −13.598 | 1.00 | 42.87 | O |
| ATOM | 2701 | N | MET | B | 243 | 27.722 | −7.195 | −11.847 | 1.00 | 45.38 | N |
| ATOM | 2702 | CA | MET | B | 243 | 27.021 | −8.110 | −10.959 | 1.00 | 46.53 | C |
| ATOM | 2703 | CB | MET | B | 243 | 26.938 | −7.521 | −9.546 | 1.00 | 47.35 | C |
| ATOM | 2704 | CG | MET | B | 243 | 26.685 | −8.551 | −8.461 | 1.00 | 48.08 | C |
| ATOM | 2705 | SD | MET | B | 243 | 26.888 | −7.892 | −6.796 | 1.00 | 49.01 | S |
| ATOM | 2706 | CE | MET | B | 243 | 26.257 | −9.269 | −5.849 | 1.00 | 47.93 | C |
| ATOM | 2707 | C | MET | B | 243 | 25.625 | −8.341 | −11.521 | 1.00 | 47.41 | C |
| ATOM | 2708 | O | MET | B | 243 | 24.946 | −7.383 | −11.888 | 1.00 | 46.69 | O |
| ATOM | 2709 | N | GLY | B | 244 | 25.222 | −9.607 | −11.623 | 1.00 | 48.28 | N |
| ATOM | 2710 | CA | GLY | B | 244 | 23.873 | −9.962 | −12.058 | 1.00 | 49.31 | C |
| ATOM | 2711 | C | GLY | B | 244 | 23.639 | −10.000 | −13.563 | 1.00 | 52.56 | C |
| ATOM | 2712 | O | GLY | B | 244 | 22.485 | −10.096 | −14.000 | 1.00 | 52.50 | O |
| ATOM | 2713 | N | GLY | B | 245 | 24.713 | −9.929 | −14.357 | 1.00 | 56.14 | N |
| ATOM | 2714 | CA | GLY | B | 245 | 24.615 | −10.133 | −15.811 | 1.00 | 57.19 | C |
| ATOM | 2715 | C | GLY | B | 245 | 24.441 | −11.607 | −16.158 | 1.00 | 58.37 | C |
| ATOM | 2716 | O | GLY | B | 245 | 24.330 | −12.455 | −15.269 | 1.00 | 55.62 | O |
| ATOM | 2717 | N | MET | B | 246 | 24.449 | −11.915 | −17.453 | 1.00 | 61.85 | N |
| ATOM | 2718 | CA | MET | B | 246 | 24.229 | −13.296 | −17.940 | 1.00 | 64.59 | C |
| ATOM | 2719 | CB | MET | B | 246 | 24.194 | −13.379 | −19.483 | 1.00 | 69.51 | C |
| ATOM | 2720 | CG | MET | B | 246 | 23.260 | −12.444 | −20.252 | 1.00 | 72.61 | C |
| ATOM | 2721 | SD | MET | B | 246 | 21.513 | −12.588 | −19.841 | 1.00 | 79.87 | S |
| ATOM | 2722 | CE | MET | B | 246 | 21.324 | −11.325 | −18.574 | 1.00 | 78.33 | C |
| ATOM | 2723 | C | MET | B | 246 | 25.359 | −14.219 | −17.473 | 1.00 | 62.24 | C |
| ATOM | 2724 | O | MET | B | 246 | 25.138 | −15.325 | −16.967 | 1.00 | 60.78 | O |
| ATOM | 2725 | N | ASN | B | 247 | 26.583 | −13.747 | −17.696 | 1.00 | 58.54 | N |
| ATOM | 2726 | CA | ASN | B | 247 | 27.809 | −14.467 | −17.345 | 1.00 | 55.62 | C |
| ATOM | 2727 | CB | ASN | B | 247 | 28.026 | −15.663 | −18.290 | 1.00 | 54.91 | C |
| ATOM | 2728 | CG | ASN | B | 247 | 28.134 | −15.247 | −19.744 | 1.00 | 53.57 | C |
| ATOM | 2729 | OD1 | ASN | B | 247 | 29.137 | −14.684 | −20.161 | 1.00 | 53.00 | O |
| ATOM | 2730 | ND2 | ASN | B | 247 | 27.099 | −15.520 | −20.519 | 1.00 | 53.77 | N |
| ATOM | 2731 | C | ASN | B | 247 | 29.018 | −13.513 | −17.388 | 1.00 | 54.43 | C |
| ATOM | 2732 | O | ASN | B | 247 | 28.893 | −12.347 | −17.794 | 1.00 | 48.72 | O |
| ATOM | 2733 | N | ARG | B | 248 | 30.179 | −14.030 | −16.989 | 1.00 | 55.50 | N |
| ATOM | 2734 | CA | ARG | B | 248 | 31.437 | −13.275 | −17.026 | 1.00 | 57.49 | C |
| ATOM | 2735 | CB | ARG | B | 248 | 32.138 | −13.348 | −15.656 | 1.00 | 62.90 | C |
| ATOM | 2736 | CG | ARG | B | 248 | 31.893 | −12.103 | −14.816 | 1.00 | 67.34 | C |
| ATOM | 2737 | CD | ARG | B | 248 | 32.091 | −12.364 | −13.325 | 1.00 | 71.91 | C |
| ATOM | 2738 | NE | ARG | B | 248 | 30.802 | −12.541 | −12.650 | 1.00 | 75.94 | N |
| ATOM | 2739 | CZ | ARG | B | 248 | 30.600 | −13.233 | −11.524 | 1.00 | 79.50 | C |
| ATOM | 2740 | NH1 | ARG | B | 248 | 31.602 | −13.857 | −10.897 | 1.00 | 80.63 | N |
| ATOM | 2741 | NH2 | ARG | B | 248 | 29.370 | −13.302 | −11.018 | 1.00 | 80.12 | N |
| ATOM | 2742 | C | ARG | B | 248 | 32.376 | −13.745 | −18.141 | 1.00 | 54.29 | C |
| ATOM | 2743 | O | ARG | B | 248 | 33.601 | −13.705 | −17.992 | 1.00 | 57.64 | O |
| ATOM | 2744 | N | SER | B | 249 | 31.819 | −14.153 | −19.277 | 1.00 | 49.61 | N |
| ATOM | 2745 | CA | SER | B | 249 | 32.642 | −14.423 | −20.453 | 1.00 | 45.66 | C |
| ATOM | 2746 | CB | SER | B | 249 | 31.858 | −15.215 | −21.505 | 1.00 | 45.49 | C |
| ATOM | 2747 | OG | SER | B | 249 | 32.664 | −15.542 | −22.618 | 1.00 | 45.06 | O |
| ATOM | 2748 | C | SER | B | 249 | 33.137 | −13.106 | −21.064 | 1.00 | 42.49 | C |
| ATOM | 2749 | O | SER | B | 249 | 32.404 | −12.106 | −21.063 | 1.00 | 40.40 | O |
| ATOM | 2750 | N | PRO | B | 250 | 34.367 | −13.098 | −21.618 | 1.00 | 39.33 | N |
| ATOM | 2751 | CA | PRO | B | 250 | 34.763 | −12.041 | −22.550 | 1.00 | 36.90 | C |
| ATOM | 2752 | CB | PRO | B | 250 | 36.173 | −12.463 | −22.994 | 1.00 | 38.17 | C |
| ATOM | 2753 | CG | PRO | B | 250 | 36.666 | −13.361 | −21.925 | 1.00 | 39.10 | C |
| ATOM | 2754 | CD | PRO | B | 250 | 35.451 | −14.075 | −21.414 | 1.00 | 40.11 | C |
| ATOM | 2755 | C | PRO | B | 250 | 33.831 | −11.966 | −23.771 | 1.00 | 33.60 | C |
| ATOM | 2756 | O | PRO | B | 250 | 33.287 | −12.973 | −24.222 | 1.00 | 31.99 | O |
| ATOM | 2757 | N | ILE | B | 251 | 33.662 | −10.762 | −24.288 | 1.00 | 30.48 | N |
| ATOM | 2758 | CA | ILE | B | 251 | 32.747 | −10.466 | −25.383 | 1.00 | 29.44 | C |
| ATOM | 2759 | CB | ILE | B | 251 | 31.935 | −9.193 | −25.037 | 1.00 | 30.89 | C |
| ATOM | 2760 | CG1 | ILE | B | 251 | 30.946 | −9.498 | −23.904 | 1.00 | 31.86 | C |
| ATOM | 2761 | CD1 | ILE | B | 251 | 30.597 | −8.313 | −23.034 | 1.00 | 33.00 | C |
| ATOM | 2762 | CG2 | ILE | B | 251 | 31.183 | −8.654 | −26.234 | 1.00 | 31.20 | C |
| ATOM | 2763 | C | ILE | B | 251 | 33.539 | −10.307 | −26.677 | 1.00 | 26.33 | C |
| ATOM | 2764 | O | ILE | B | 251 | 34.656 | −9.818 | −26.671 | 1.00 | 26.67 | O |
| ATOM | 2765 | N | LEU | B | 252 | 32.953 | −10.758 | −27.776 | 1.00 | 23.99 | N |
| ATOM | 2766 | CA | LEU | B | 252 | 33.505 | −10.587 | −29.132 | 1.00 | 22.16 | C |
| ATOM | 2767 | CB | LEU | B | 252 | 33.586 | −11.949 | −29.829 | 1.00 | 22.18 | C |
| ATOM | 2768 | CG | LEU | B | 252 | 34.290 | −12.098 | −31.182 | 1.00 | 22.59 | C |
| ATOM | 2769 | CD1 | LEU | B | 252 | 35.773 | −11.751 | −31.152 | 1.00 | 22.87 | C |
| ATOM | 2770 | CD2 | LEU | B | 252 | 34.136 | −13.525 | −31.694 | 1.00 | 22.69 | C |
| ATOM | 2771 | C | LEU | B | 252 | 32.558 | −9.699 | −29.908 | 1.00 | 19.94 | C |
| ATOM | 2772 | O | LEU | B | 252 | 31.354 | −9.945 | −29.900 | 1.00 | 19.71 | O |
| ATOM | 2773 | N | THR | B | 253 | 33.090 | −8.660 | −30.540 | 1.00 | 17.82 | N |
| ATOM | 2774 | CA | THR | B | 253 | 32.306 | −7.792 | −31.423 | 1.00 | 17.04 | C |
| ATOM | 2775 | CB | THR | B | 253 | 32.698 | −6.306 | −31.253 | 1.00 | 16.18 | C |
| ATOM | 2776 | OG1 | THR | B | 253 | 32.466 | −5.907 | −29.896 | 1.00 | 15.91 | O |
| ATOM | 2777 | CG2 | THR | B | 253 | 31.904 | −5.419 | −32.197 | 1.00 | 16.12 | C |
| ATOM | 2778 | C | THR | B | 253 | 32.536 | −8.232 | −32.861 | 1.00 | 16.81 | C |
| ATOM | 2779 | O | THR | B | 253 | 33.682 | −8.372 | −33.293 | 1.00 | 16.86 | O |
| ATOM | 2780 | N | ILE | B | 254 | 31.448 | −8.446 | −33.597 | 1.00 | 16.88 | N |
| ATOM | 2781 | CA | ILE | B | 254 | 31.498 | −8.859 | −34.999 | 1.00 | 17.09 | C |
| ATOM | 2782 | CB | ILE | B | 254 | 30.618 | −10.097 | −35.254 | 1.00 | 17.06 | C |
| ATOM | 2783 | CG1 | ILE | B | 254 | 31.177 | −11.296 | −34.482 | 1.00 | 17.55 | C |
| ATOM | 2784 | CD1 | ILE | B | 254 | 30.217 | −12.420 | −34.240 | 1.00 | 18.02 | C |
| ATOM | 2785 | CG2 | ILE | B | 254 | 30.555 | −10.418 | −36.745 | 1.00 | 17.01 | C |
| ATOM | 2786 | C | ILE | B | 254 | 30.985 | −7.704 | −35.846 | 1.00 | 17.46 | C |
| ATOM | 2787 | O | ILE | B | 254 | 29.830 | −7.303 | −35.705 | 1.00 | 17.09 | O |
| ATOM | 2788 | N | ILE | B | 255 | 31.849 | −7.191 | −36.721 | 1.00 | 17.99 | N |
| ATOM | 2789 | CA | ILE | B | 255 | 31.511 | −6.127 | −37.655 | 1.00 | 18.26 | C |
| ATOM | 2790 | CB | ILE | B | 255 | 32.613 | −5.042 | −37.731 | 1.00 | 18.14 | C |
| ATOM | 2791 | CG1 | ILE | B | 255 | 32.970 | −4.509 | −36.336 | 1.00 | 18.37 | C |
| ATOM | 2792 | CD1 | ILE | B | 255 | 31.815 | −3.899 | −35.559 | 1.00 | 18.70 | C |
| ATOM | 2793 | CG2 | ILE | B | 255 | 32.185 | −3.896 | −38.643 | 1.00 | 17.91 | C |
| ATOM | 2794 | C | ILE | B | 255 | 31.344 | −6.774 | −39.023 | 1.00 | 18.62 | C |
| ATOM | 2795 | O | ILE | B | 255 | 32.272 | −7.408 | −39.524 | 1.00 | 18.66 | O |
| ATOM | 2796 | N | THR | B | 256 | 30.160 | −6.619 | −39.615 | 1.00 | 19.44 | N |
| ATOM | 2797 | CA | THR | B | 256 | 29.884 | −7.128 | −40.948 | 1.00 | 20.58 | C |
| ATOM | 2798 | CB | THR | B | 256 | 28.729 | −8.151 | −40.958 | 1.00 | 20.99 | C |
| ATOM | 2799 | OG1 | THR | B | 256 | 27.593 | −7.605 | −40.280 | 1.00 | 21.91 | O |
| ATOM | 2800 | CG2 | THR | B | 256 | 29.131 | −9.442 | −40.263 | 1.00 | 21.06 | C |
| ATOM | 2801 | C | THR | B | 256 | 29.542 | −5.987 | −41.894 | 1.00 | 21.87 | C |
| ATOM | 2802 | O | THR | B | 256 | 28.921 | −4.999 | −41.508 | 1.00 | 21.67 | O |
| ATOM | 2803 | N | LEU | B | 257 | 29.968 | −6.146 | −43.144 | 1.00 | 23.49 | N |
| ATOM | 2804 | CA | LEU | B | 257 | 29.576 | −5.275 | −44.231 | 1.00 | 24.64 | C |
| ATOM | 2805 | CB | LEU | B | 257 | 30.789 | −5.019 | −45.124 | 1.00 | 25.77 | C |
| ATOM | 2806 | CG | LEU | B | 257 | 30.851 | −3.740 | −45.950 | 1.00 | 26.69 | C |
| ATOM | 2807 | CD1 | LEU | B | 257 | 30.875 | −2.505 | −45.053 | 1.00 | 27.61 | C |
| ATOM | 2808 | CD2 | LEU | B | 257 | 32.108 | −3.785 | −46.818 | 1.00 | 26.58 | C |
| ATOM | 2809 | C | LEU | B | 257 | 28.457 | −5.989 | −44.996 | 1.00 | 24.94 | C |
| ATOM | 2810 | O | LEU | B | 257 | 28.613 | −7.150 | −45.391 | 1.00 | 24.60 | O |
| ATOM | 2811 | N | GLU | B | 258 | 27.318 | −5.309 | −45.169 | 1.00 | 25.99 | N |
| ATOM | 2812 | CA | GLU | B | 258 | 26.150 | −5.853 | −45.885 | 1.00 | 26.60 | C |
| ATOM | 2813 | CB | GLU | B | 258 | 24.923 | −5.955 | −44.966 | 1.00 | 27.19 | C |
| ATOM | 2814 | CG | GLU | B | 258 | 25.147 | −6.476 | −43.556 | 1.00 | 27.43 | C |
| ATOM | 2815 | CD | GLU | B | 258 | 23.861 | −6.516 | −42.746 | 1.00 | 27.45 | C |
| ATOM | 2816 | OE1 | GLU | B | 258 | 22.992 | −5.631 | −42.931 | 1.00 | 28.26 | O |
| ATOM | 2817 | OE2 | GLU | B | 258 | 23.706 | −7.453 | −41.928 | 1.00 | 27.93 | O |
| ATOM | 2818 | C | GLU | B | 258 | 25.742 | −4.923 | −47.017 | 1.00 | 26.91 | C |
| ATOM | 2819 | O | GLU | B | 258 | 25.950 | −3.710 | −46.915 | 1.00 | 26.03 | O |
| ATOM | 2820 | N | ASP | B | 259 | 25.093 | −5.472 | −48.051 | 1.00 | 27.32 | N |
| ATOM | 2821 | CA | ASP | B | 259 | 24.431 | −4.637 | −49.072 | 1.00 | 29.24 | C |
| ATOM | 2822 | CB | ASP | B | 259 | 24.326 | −5.358 | −50.437 | 1.00 | 30.23 | C |
| ATOM | 2823 | CG | ASP | B | 259 | 23.336 | −6.535 | −50.454 | 1.00 | 32.43 | C |
| ATOM | 2824 | OD1 | ASP | B | 259 | 22.504 | −6.693 | −49.535 | 1.00 | 36.21 | O |
| ATOM | 2825 | OD2 | ASP | B | 259 | 23.389 | −7.317 | −51.422 | 1.00 | 34.74 | O |
| ATOM | 2826 | C | ASP | B | 259 | 23.069 | −4.128 | −48.561 | 1.00 | 29.31 | C |
| ATOM | 2827 | O | ASP | B | 259 | 22.679 | −4.429 | −47.437 | 1.00 | 29.27 | O |
| ATOM | 2828 | N | SER | B | 260 | 22.355 | −3.364 | −49.383 | 1.00 | 30.16 | N |
| ATOM | 2829 | CA | SER | B | 260 | 21.050 | −2.822 | −48.999 | 1.00 | 31.44 | C |
| ATOM | 2830 | CB | SER | B | 260 | 20.531 | −1.857 | −50.063 | 1.00 | 32.17 | C |
| ATOM | 2831 | OG | SER | B | 260 | 20.577 | −2.444 | −51.355 | 1.00 | 33.27 | O |
| ATOM | 2832 | C | SER | B | 260 | 19.998 | −3.901 | −48.690 | 1.00 | 31.68 | C |
| ATOM | 2833 | O | SER | B | 260 | 19.121 | −3.675 | −47.863 | 1.00 | 31.61 | O |
| ATOM | 2834 | N | SER | B | 261 | 20.109 | −5.066 | −49.335 | 1.00 | 31.51 | N |
| ATOM | 2835 | CA | SER | B | 261 | 19.197 | −6.193 | −49.102 | 1.00 | 31.43 | C |
| ATOM | 2836 | CB | SER | B | 261 | 18.985 | −6.971 | −50.409 | 1.00 | 31.69 | C |
| ATOM | 2837 | OG | SER | B | 261 | 18.599 | −6.102 | −51.459 | 1.00 | 30.61 | O |
| ATOM | 2838 | C | SER | B | 261 | 19.657 | −7.161 | −48.005 | 1.00 | 31.55 | C |
| ATOM | 2839 | O | SER | B | 261 | 19.045 | −8.212 | −47.823 | 1.00 | 31.29 | O |
| ATOM | 2840 | N | GLY | B | 262 | 20.726 | −6.823 | −47.285 | 1.00 | 32.10 | N |
| ATOM | 2841 | CA | GLY | B | 262 | 21.198 | −7.630 | −46.152 | 1.00 | 31.79 | C |
| ATOM | 2842 | C | GLY | B | 262 | 22.159 | −8.773 | −46.451 | 1.00 | 31.79 | C |
| ATOM | 2843 | O | GLY | B | 262 | 22.485 | −9.537 | −45.545 | 1.00 | 33.51 | O |
| ATOM | 2844 | N | ASN | B | 263 | 22.623 | −8.894 | −47.696 | 1.00 | 31.52 | N |
| ATOM | 2845 | CA | ASN | B | 263 | 23.586 | −9.948 | −48.069 | 1.00 | 30.58 | C |
| ATOM | 2846 | CB | ASN | B | 263 | 23.632 | −10.156 | −49.589 | 1.00 | 30.74 | C |
| ATOM | 2847 | CG | ASN | B | 263 | 22.290 | −10.574 | −50.168 | 1.00 | 31.18 | C |
| ATOM | 2848 | OD1 | ASN | B | 263 | 21.778 | −11.668 | −49.889 | 1.00 | 31.48 | O |
| ATOM | 2849 | ND2 | ASN | B | 263 | 21.708 | −9.704 | −50.970 | 1.00 | 31.63 | N |
| ATOM | 2850 | C | ASN | B | 263 | 24.988 | −9.598 | −47.575 | 1.00 | 29.50 | C |
| ATOM | 2851 | O | ASN | B | 263 | 25.411 | −8.447 | −47.663 | 1.00 | 29.28 | O |
| ATOM | 2852 | N | LEU | B | 264 | 25.695 | −10.601 | −47.072 | 1.00 | 28.14 | N |
| ATOM | 2853 | CA | LEU | B | 264 | 27.036 | −10.445 | −46.504 | 1.00 | 29.22 | C |
| ATOM | 2854 | CB | LEU | B | 264 | 27.433 | −11.758 | −45.813 | 1.00 | 29.48 | C |
| ATOM | 2855 | CG | LEU | B | 264 | 28.841 | −11.851 | −45.227 | 1.00 | 30.04 | C |
| ATOM | 2856 | CD1 | LEU | B | 264 | 28.866 | −11.052 | −43.941 | 1.00 | 29.27 | C |
| ATOM | 2857 | CD2 | LEU | B | 264 | 29.278 | −13.297 | −45.006 | 1.00 | 30.59 | C |
| ATOM | 2858 | C | LEU | B | 264 | 28.080 | −10.123 | −47.578 | 1.00 | 29.06 | C |
| ATOM | 2859 | O | LEU | B | 264 | 28.205 | −10.851 | −48.561 | 1.00 | 31.03 | O |
| ATOM | 2860 | N | LEU | B | 265 | 28.812 | −9.029 | −47.380 | 1.00 | 27.65 | N |
| ATOM | 2861 | CA | LEU | B | 265 | 29.940 | −8.648 | −48.224 | 1.00 | 26.37 | C |
| ATOM | 2862 | CB | LEU | B | 265 | 29.837 | −7.167 | −48.589 | 1.00 | 26.26 | C |
| ATOM | 2863 | CG | LEU | B | 265 | 28.493 | −6.714 | −49.181 | 1.00 | 26.71 | C |
| ATOM | 2864 | CD1 | LEU | B | 265 | 28.532 | −5.247 | −49.568 | 1.00 | 26.47 | C |
| ATOM | 2865 | CD2 | LEU | B | 265 | 28.100 | −7.549 | −50.394 | 1.00 | 26.55 | C |
| ATOM | 2866 | C | LEU | B | 265 | 31.294 | −8.919 | −47.547 | 1.00 | 26.31 | C |
| ATOM | 2867 | O | LEU | B | 265 | 32.291 | −9.189 | −48.226 | 1.00 | 25.33 | O |
| ATOM | 2868 | N | GLY | B | 266 | 31.347 | −8.814 | −46.218 | 1.00 | 25.24 | N |
| ATOM | 2869 | CA | GLY | B | 266 | 32.601 | −8.968 | −45.484 | 1.00 | 24.76 | C |
| ATOM | 2870 | C | GLY | B | 266 | 32.398 | −9.032 | −43.989 | 1.00 | 23.67 | C |
| ATOM | 2871 | O | GLY | B | 266 | 31.368 | −8.591 | −43.489 | 1.00 | 23.80 | O |
| ATOM | 2872 | N | ARG | B | 267 | 33.388 | −9.570 | −43.283 | 1.00 | 23.30 | N |
| ATOM | 2873 | CA | ARG | B | 267 | 33.314 | −9.755 | −41.840 | 1.00 | 23.88 | C |
| ATOM | 2874 | CB | ARG | B | 267 | 32.636 | −11.098 | −41.495 | 1.00 | 23.86 | C |
| ATOM | 2875 | CG | ARG | B | 267 | 32.298 | −11.246 | −40.013 | 1.00 | 23.22 | C |
| ATOM | 2876 | CD | ARG | B | 267 | 31.926 | −12.641 | −39.593 | 1.00 | 23.22 | C |
| ATOM | 2877 | NE | ARG | B | 267 | 30.668 | −13.111 | −40.195 | 1.00 | 23.01 | N |
| ATOM | 2878 | CZ | ARG | B | 267 | 30.548 | −14.045 | −41.144 | 1.00 | 23.73 | C |
| ATOM | 2879 | NH1 | ARG | B | 267 | 31.605 | −14.649 | −41.687 | 1.00 | 23.71 | N |
| ATOM | 2880 | NH2 | ARG | B | 267 | 29.338 | −14.372 | −41.569 | 1.00 | 24.50 | N |
| ATOM | 2881 | C | ARG | B | 267 | 34.689 | −9.704 | −41.186 | 1.00 | 24.35 | C |
| ATOM | 2882 | O | ARG | B | 267 | 35.667 | −10.225 | −41.724 | 1.00 | 25.78 | O |
| ATOM | 2883 | N | ASN | B | 268 | 34.742 | −9.066 | −40.019 | 1.00 | 24.39 | N |
| ATOM | 2884 | CA | ASN | B | 268 | 35.904 | −9.109 | −39.129 | 1.00 | 24.47 | C |
| ATOM | 2885 | CB | ASN | B | 268 | 36.810 | −7.912 | −39.382 | 1.00 | 26.32 | C |
| ATOM | 2886 | CG | ASN | B | 268 | 37.780 | −8.160 | −40.505 | 1.00 | 28.22 | C |
| ATOM | 2887 | OD1 | ASN | B | 268 | 38.708 | −8.947 | −40.352 | 1.00 | 30.63 | O |
| ATOM | 2888 | ND2 | ASN | B | 268 | 37.563 | −7.511 | −41.648 | 1.00 | 28.80 | N |
| ATOM | 2889 | C | ASN | B | 268 | 35.395 | −9.099 | −37.700 | 1.00 | 22.78 | C |
| ATOM | 2890 | O | ASN | B | 268 | 34.195 | −8.959 | −37.454 | 1.00 | 22.35 | O |
| ATOM | 2891 | N | SER | B | 269 | 36.304 | −9.250 | −36.752 | 1.00 | 21.72 | N |
| ATOM | 2892 | CA | SER | B | 269 | 35.919 | −9.268 | −35.349 | 1.00 | 21.58 | C |
| ATOM | 2893 | CB | SER | B | 269 | 35.331 | −10.634 | −34.984 | 1.00 | 21.78 | C |
| ATOM | 2894 | OG | SER | B | 269 | 36.285 | −11.658 | −35.203 | 1.00 | 22.11 | O |
| ATOM | 2895 | C | SER | B | 269 | 37.080 | −8.950 | −34.422 | 1.00 | 21.49 | C |
| ATOM | 2896 | O | SER | B | 269 | 38.248 | −9.046 | −34.799 | 1.00 | 21.52 | O |
| ATOM | 2897 | N | PHE | B | 270 | 36.730 | −8.584 | −33.198 | 1.00 | 20.92 | N |
| ATOM | 2898 | CA | PHE | B | 270 | 37.693 | −8.317 | −32.155 | 1.00 | 20.89 | C |
| ATOM | 2899 | CB | PHE | B | 270 | 38.264 | −6.895 | −32.277 | 1.00 | 20.42 | C |
| ATOM | 2900 | CG | PHE | B | 270 | 37.234 | −5.797 | −32.241 | 1.00 | 20.19 | C |
| ATOM | 2901 | CD1 | PHE | B | 270 | 36.560 | −5.418 | −33.390 | 1.00 | 20.11 | C |
| ATOM | 2902 | CE1 | PHE | B | 270 | 35.626 | −4.381 | −33.368 | 1.00 | 19.87 | C |
| ATOM | 2903 | CZ | PHE | B | 270 | 35.369 | −3.708 | −32.186 | 1.00 | 19.67 | C |
| ATOM | 2904 | CE2 | PHE | B | 270 | 36.052 | −4.056 | −31.034 | 1.00 | 19.90 | C |
| ATOM | 2905 | CD2 | PHE | B | 270 | 36.983 | −5.095 | −31.065 | 1.00 | 20.25 | C |
| ATOM | 2906 | C | PHE | B | 270 | 37.073 | −8.536 | −30.782 | 1.00 | 21.52 | C |
| ATOM | 2907 | O | PHE | B | 270 | 35.867 | −8.402 | −30.594 | 1.00 | 21.96 | O |
| ATOM | 2908 | N | GLU | B | 271 | 37.919 | −8.902 | −29.832 | 1.00 | 22.46 | N |
| ATOM | 2909 | CA | GLU | B | 271 | 37.520 | −9.078 | −28.451 | 1.00 | 23.93 | C |
| ATOM | 2910 | CB | GLU | B | 271 | 38.558 | −9.924 | −27.732 | 1.00 | 26.16 | C |
| ATOM | 2911 | CG | GLU | B | 271 | 38.118 | −10.448 | −26.371 | 1.00 | 28.79 | C |
| ATOM | 2912 | CD | GLU | B | 271 | 39.051 | −11.510 | −25.808 | 1.00 | 30.67 | C |
| ATOM | 2913 | OE1 | GLU | B | 271 | 39.658 | −12.288 | −26.585 | 1.00 | 32.34 | O |
| ATOM | 2914 | OE2 | GLU | B | 271 | 39.256 | −11.521 | −24.586 | 1.00 | 32.83 | O |
| ATOM | 2915 | C | GLU | B | 271 | 37.374 | −7.696 | −27.829 | 1.00 | 23.84 | C |
| ATOM | 2916 | O | GLU | B | 271 | 38.065 | −6.764 | −28.224 | 1.00 | 23.35 | O |
| ATOM | 2917 | N | VAL | B | 272 | 36.463 | −7.556 | −26.872 | 1.00 | 24.06 | N |
| ATOM | 2918 | CA | VAL | B | 272 | 36.273 | −6.298 | −26.159 | 1.00 | 24.96 | C |
| ATOM | 2919 | CB | VAL | B | 272 | 35.062 | −5.490 | −26.703 | 1.00 | 24.81 | C |
| ATOM | 2920 | CG1 | VAL | B | 272 | 34.894 | −4.138 | −26.014 | 1.00 | 24.71 | C |
| ATOM | 2921 | CG2 | VAL | B | 272 | 33.801 | −6.296 | −26.642 | 1.00 | 25.22 | C |
| ATOM | 2922 | C | VAL | B | 272 | 36.191 | −6.538 | −24.649 | 1.00 | 25.94 | C |
| ATOM | 2923 | O | VAL | B | 272 | 35.622 | −7.514 | −24.193 | 1.00 | 26.87 | O |
| ATOM | 2924 | N | ARG | B | 273 | 36.815 | −5.632 | −23.897 | 1.00 | 26.23 | N |
| ATOM | 2925 | CA | ARG | B | 273 | 36.736 | −5.581 | −22.449 | 1.00 | 26.39 | C |
| ATOM | 2926 | CB | ARG | B | 273 | 38.056 | −6.010 | −21.815 | 1.00 | 27.45 | C |
| ATOM | 2927 | CG | ARG | B | 273 | 38.029 | −5.961 | −20.294 | 1.00 | 28.56 | C |
| ATOM | 2928 | CD | ARG | B | 273 | 39.368 | −6.280 | −19.667 | 1.00 | 30.10 | C |
| ATOM | 2929 | NE | ARG | B | 273 | 39.333 | −5.985 | −18.238 | 1.00 | 32.66 | N |
| ATOM | 2930 | CZ | ARG | B | 273 | 38.758 | −6.757 | −17.306 | 1.00 | 34.65 | C |
| ATOM | 2931 | NH1 | ARG | B | 273 | 38.150 | −7.905 | −17.629 | 1.00 | 35.11 | N |
| ATOM | 2932 | NH2 | ARG | B | 273 | 38.778 | −6.375 | −16.028 | 1.00 | 35.48 | N |
| ATOM | 2933 | C | ARG | B | 273 | 36.407 | −4.152 | −22.032 | 1.00 | 26.29 | C |
| ATOM | 2934 | O | ARG | B | 273 | 37.153 | −3.225 | −22.341 | 1.00 | 25.67 | O |
| ATOM | 2935 | N | VAL | B | 274 | 35.291 | −4.000 | −21.330 | 1.00 | 26.02 | N |
| ATOM | 2936 | CA | VAL | B | 274 | 34.902 | −2.732 | −20.731 | 1.00 | 26.43 | C |
| ATOM | 2937 | CB | VAL | B | 274 | 33.369 | −2.556 | −20.725 | 1.00 | 26.28 | C |
| ATOM | 2938 | CG1 | VAL | B | 274 | 33.007 | −1.159 | −20.254 | 1.00 | 26.68 | C |
| ATOM | 2939 | CG2 | VAL | B | 274 | 32.794 | −2.841 | −22.107 | 1.00 | 25.80 | C |
| ATOM | 2940 | C | VAL | B | 274 | 35.446 | −2.733 | −19.300 | 1.00 | 26.76 | C |
| ATOM | 2941 | O | VAL | B | 274 | 35.208 | −3.676 | −18.553 | 1.00 | 25.92 | O |
| ATOM | 2942 | N | CYS | B | 275 | 36.199 | −1.690 | −18.944 | 1.00 | 26.80 | N |
| ATOM | 2943 | CA | CYS | B | 275 | 36.912 | −1.629 | −17.661 | 1.00 | 26.85 | C |
| ATOM | 2944 | CB | CYS | B | 275 | 38.192 | −2.468 | −17.725 | 1.00 | 27.38 | C |
| ATOM | 2945 | SG | CYS | B | 275 | 39.282 | −2.086 | −19.123 | 1.00 | 27.06 | S |
| ATOM | 2946 | C | CYS | B | 275 | 37.244 | −0.191 | −17.252 | 1.00 | 27.90 | C |
| ATOM | 2947 | O | CYS | B | 275 | 37.121 | 0.736 | −18.044 | 1.00 | 26.51 | O |
| ATOM | 2948 | N | ALA | B | 276 | 37.679 | −0.029 | −16.004 | 1.00 | 28.22 | N |
| ATOM | 2949 | CA | ALA | B | 276 | 38.003 | 1.279 | −15.431 | 1.00 | 28.92 | C |
| ATOM | 2950 | CB | ALA | B | 276 | 38.129 | 1.162 | −13.919 | 1.00 | 29.43 | C |
| ATOM | 2951 | C | ALA | B | 276 | 39.271 | 1.909 | −16.010 | 1.00 | 30.21 | C |
| ATOM | 2952 | O | ALA | B | 276 | 39.274 | 3.105 | −16.323 | 1.00 | 29.14 | O |
| ATOM | 2953 | N | CYS | B | 277 | 40.335 | 1.115 | −16.140 | 1.00 | 32.23 | N |
| ATOM | 2954 | CA | CYS | B | 277 | 41.637 | 1.604 | −16.614 | 1.00 | 34.22 | C |
| ATOM | 2955 | CB | CYS | B | 277 | 42.665 | 1.555 | −15.474 | 1.00 | 38.09 | C |
| ATOM | 2956 | SG | CYS | B | 277 | 42.124 | 2.182 | −13.866 | 1.00 | 47.28 | S |
| ATOM | 2957 | C | CYS | B | 277 | 42.143 | 0.785 | −17.816 | 1.00 | 31.50 | C |
| ATOM | 2958 | O | CYS | B | 277 | 42.928 | −0.153 | −17.640 | 1.00 | 30.60 | O |
| ATOM | 2959 | N | PRO | B | 278 | 41.687 | 1.129 | −19.041 | 1.00 | 30.20 | N |
| ATOM | 2960 | CA | PRO | B | 278 | 42.101 | 0.422 | −20.274 | 1.00 | 30.01 | C |
| ATOM | 2961 | CB | PRO | B | 278 | 41.502 | 1.279 | −21.390 | 1.00 | 29.93 | C |
| ATOM | 2962 | CG | PRO | B | 278 | 40.311 | 1.909 | −20.774 | 1.00 | 30.48 | C |
| ATOM | 2963 | CD | PRO | B | 278 | 40.597 | 2.085 | −19.313 | 1.00 | 30.15 | C |
| ATOM | 2964 | C | PRO | B | 278 | 43.612 | 0.282 | −20.481 | 1.00 | 28.64 | C |
| ATOM | 2965 | O | PRO | B | 278 | 44.092 | −0.814 | −20.788 | 1.00 | 26.62 | O |
| ATOM | 2966 | N | GLY | B | 279 | 44.338 | 1.380 | −20.309 | 1.00 | 28.52 | N |
| ATOM | 2967 | CA | GLY | B | 279 | 45.798 | 1.390 | −20.431 | 1.00 | 29.92 | C |
| ATOM | 2968 | C | GLY | B | 279 | 46.470 | 0.340 | −19.565 | 1.00 | 30.39 | C |
| ATOM | 2969 | O | GLY | B | 279 | 47.229 | −0.490 | −20.063 | 1.00 | 30.75 | O |
| ATOM | 2970 | N | ARG | B | 280 | 46.151 | 0.362 | −18.272 | 1.00 | 31.33 | N |
| ATOM | 2971 | CA | ARG | B | 280 | 46.755 | −0.558 | −17.305 | 1.00 | 33.04 | C |
| ATOM | 2972 | CB | ARG | B | 280 | 46.381 | −0.162 | −15.874 | 1.00 | 36.15 | C |
| ATOM | 2973 | CG | ARG | B | 280 | 47.126 | −0.950 | −14.799 | 1.00 | 39.07 | C |
| ATOM | 2974 | CD | ARG | B | 280 | 46.712 | −0.670 | −13.373 | 1.00 | 42.15 | C |
| ATOM | 2975 | NE | ARG | B | 280 | 45.340 | −1.088 | −13.024 | 1.00 | 44.55 | N |
| ATOM | 2976 | CZ | ARG | B | 280 | 44.369 | −0.284 | −12.572 | 1.00 | 48.41 | C |
| ATOM | 2977 | NH1 | ARG | B | 280 | 44.582 | 1.021 | −12.371 | 1.00 | 48.56 | N |
| ATOM | 2978 | NH2 | ARG | B | 280 | 43.165 | −0.794 | −12.312 | 1.00 | 48.37 | N |
| ATOM | 2979 | C | ARG | B | 280 | 46.352 | −2.008 | −17.600 | 1.00 | 31.73 | C |
| ATOM | 2980 | O | ARG | B | 280 | 47.200 | −2.905 | −17.618 | 1.00 | 32.40 | O |
| ATOM | 2981 | N | ASP | B | 281 | 45.061 | −2.229 | −17.832 | 1.00 | 28.98 | N |
| ATOM | 2982 | CA | ASP | B | 281 | 44.550 | −3.571 | −18.145 | 1.00 | 28.03 | C |
| ATOM | 2983 | CB | ASP | B | 281 | 43.014 | −3.594 | −18.166 | 1.00 | 28.40 | C |
| ATOM | 2984 | CG | ASP | B | 281 | 42.392 | −3.529 | −16.758 | 1.00 | 29.55 | C |
| ATOM | 2985 | OD1 | ASP | B | 281 | 43.116 | −3.639 | −15.743 | 1.00 | 29.60 | O |
| ATOM | 2986 | OD2 | ASP | B | 281 | 41.159 | −3.367 | −16.652 | 1.00 | 29.43 | O |
| ATOM | 2987 | C | ASP | B | 281 | 45.113 | −4.139 | −19.453 | 1.00 | 27.25 | C |
| ATOM | 2988 | O | ASP | B | 281 | 45.343 | −5.344 | −19.539 | 1.00 | 27.59 | O |
| ATOM | 2989 | N | ARG | B | 282 | 45.335 | −3.290 | −20.461 | 1.00 | 26.44 | N |
| ATOM | 2990 | CA | ARG | B | 282 | 46.027 | −3.733 | −21.678 | 1.00 | 25.87 | C |
| ATOM | 2991 | CB | ARG | B | 282 | 45.998 | −2.673 | −22.790 | 1.00 | 24.32 | C |
| ATOM | 2992 | CG | ARG | B | 282 | 46.792 | −3.089 | −24.035 | 1.00 | 23.90 | C |
| ATOM | 2993 | CD | ARG | B | 282 | 46.696 | −2.109 | −25.198 | 1.00 | 23.39 | C |
| ATOM | 2994 | NE | ARG | B | 282 | 47.781 | −2.363 | −26.146 | 1.00 | 22.97 | N |
| ATOM | 2995 | CZ | ARG | B | 282 | 48.281 | −1.496 | −27.025 | 1.00 | 22.82 | C |
| ATOM | 2996 | NH1 | ARG | B | 282 | 47.805 | −0.265 | −27.141 | 1.00 | 23.53 | N |
| ATOM | 2997 | NH2 | ARG | B | 282 | 49.272 | −1.885 | −27.821 | 1.00 | 22.34 | N |
| ATOM | 2998 | C | ARG | B | 282 | 47.471 | −4.120 | −21.362 | 1.00 | 25.56 | C |
| ATOM | 2999 | O | ARG | B | 282 | 47.917 | −5.207 | −21.729 | 1.00 | 25.29 | O |
| ATOM | 3000 | N | ARG | B | 283 | 48.185 | −3.233 | −20.675 | 1.00 | 26.23 | N |
| ATOM | 3001 | CA | ARG | B | 283 | 49.594 | −3.477 | −20.315 | 1.00 | 27.28 | C |
| ATOM | 3002 | CB | ARG | B | 283 | 50.202 | −2.267 | −19.594 | 1.00 | 26.39 | C |
| ATOM | 3003 | CG | ARG | B | 283 | 51.723 | −2.232 | −19.633 | 1.00 | 25.39 | C |
| ATOM | 3004 | CD | ARG | B | 283 | 52.279 | −1.014 | −18.913 | 1.00 | 24.62 | C |
| ATOM | 3005 | NE | ARG | B | 263 | 51.896 | 0.259 | −19.526 | 1.00 | 23.85 | N |
| ATOM | 3006 | CZ | ARG | B | 283 | 52.200 | 1.468 | −19.036 | 1.00 | 23.83 | C |
| ATOM | 3007 | NH1 | ARG | B | 283 | 51.800 | 2.566 | −19.667 | 1.00 | 22.73 | N |
| ATOM | 3008 | NH2 | ARG | B | 283 | 52.919 | 1.599 | −17.919 | 1.00 | 23.58 | N |
| ATOM | 3009 | C | ARG | B | 283 | 49.780 | −4.753 | −19.474 | 1.00 | 28.74 | C |
| ATOM | 3010 | O | ARG | B | 283 | 50.760 | −5.472 | −19.662 | 1.00 | 30.43 | O |
| ATOM | 3011 | N | THR | B | 284 | 48.831 | −5.029 | −18.581 | 1.00 | 29.09 | N |
| ATOM | 3012 | CA | THR | B | 284 | 48.854 | −6.235 | −17.752 | 1.00 | 30.10 | C |
| ATOM | 3013 | CB | THR | B | 264 | 47.816 | −6.155 | −16.612 | 1.00 | 30.66 | C |
| ATOM | 3014 | OG1 | THR | B | 284 | 48.005 | −4.950 | −15.873 | 1.00 | 30.60 | O |
| ATOM | 3015 | CG2 | THR | B | 284 | 47.927 | −7.362 | −15.669 | 1.00 | 30.77 | C |
| ATOM | 3016 | C | THR | B | 284 | 48.589 | −7.511 | −18.561 | 1.00 | 31.06 | C |
| ATOM | 3017 | O | THR | B | 284 | 49.325 | −8.487 | −18.431 | 1.00 | 31.33 | O |
| ATOM | 3018 | N | GLU | B | 285 | 47.544 | −7.512 | −19.366 | 1.00 | 32.02 | N |
| ATOM | 3019 | CA | GLU | B | 285 | 47.237 | −8.680 | −20.232 | 1.00 | 33.48 | C |
| ATOM | 3020 | CB | GLU | B | 285 | 45.863 | −8.546 | −20.906 | 1.00 | 33.41 | C |
| ATOM | 3021 | CG | GLU | B | 285 | 44.697 | −8.688 | −19.933 | 1.00 | 33.40 | C |
| ATOM | 3022 | CD | GLU | B | 285 | 43.338 | −8.795 | −20.594 | 1.00 | 34.12 | C |
| ATOM | 3023 | OE1 | GLU | B | 285 | 43.254 | −9.239 | −21.756 | 1.00 | 35.05 | O |
| ATOM | 3024 | OE2 | GLU | B | 285 | 42.333 | −8.443 | −19.944 | 1.00 | 33.86 | O |
| ATOM | 3025 | C | GLU | B | 285 | 48.314 | −8.970 | −21.281 | 1.00 | 34.90 | C |
| ATOM | 3026 | O | GLU | B | 285 | 48.487 | −10.115 | −21.690 | 1.00 | 35.85 | O |
| ATOM | 3027 | N | GLU | B | 286 | 49.023 | −7.933 | −21.722 | 1.00 | 36.12 | N |
| ATOM | 3028 | CA | GLU | B | 286 | 50.142 | −8.093 | −22.670 | 1.00 | 38.59 | C |
| ATOM | 3029 | CB | GLU | B | 286 | 50.407 | −6.781 | −23.420 | 1.00 | 37.87 | C |
| ATOM | 3030 | CG | GLU | B | 286 | 49.345 | −6.495 | −24.479 | 1.00 | 37.50 | C |
| ATOM | 3031 | CD | GLU | B | 286 | 49.577 | −5.217 | −25.257 | 1.00 | 35.72 | C |
| ATOM | 3032 | OE1 | GLU | B | 286 | 50.534 | −4.479 | −24.975 | 1.00 | 36.22 | O |
| ATOM | 3033 | OE2 | GLU | B | 286 | 48.778 | −4.949 | −26.174 | 1.00 | 33.74 | O |
| ATOM | 3034 | C | GLU | B | 286 | 51.426 | −8.623 | −22.020 | 1.00 | 42.35 | C |
| ATOM | 3035 | O | GLU | B | 286 | 52.256 | −9.223 | −22.690 | 1.00 | 42.26 | O |
| ATOM | 3036 | N | GLU | B | 287 | 51.592 | −8.371 | −20.725 | 1.00 | 48.91 | N |
| ATOM | 3037 | CA | GLU | B | 287 | 52.670 | −8.951 | −19.908 | 1.00 | 53.68 | C |
| ATOM | 3038 | CB | GLU | B | 287 | 52.705 | −8.194 | −18.545 | 1.00 | 57.26 | C |
| ATOM | 3039 | CG | GLU | B | 287 | 53.204 | −8.849 | −17.256 | 1.00 | 60.62 | C |
| ATOM | 3040 | CD | GLU | B | 287 | 54.649 | −9.268 | −17.299 | 1.00 | 65.85 | C |
| ATOM | 3041 | OE1 | GLU | B | 287 | 55.377 | −8.899 | −18.243 | 1.00 | 71.45 | O |
| ATOM | 3042 | OE2 | GLU | B | 287 | 55.052 | −9.976 | −16.366 | 1.00 | 66.65 | O |
| ATOM | 3043 | C | GLU | B | 287 | 52.509 | −10.472 | −19.750 | 1.00 | 55.98 | C |
| ATOM | 3044 | O | GLU | B | 287 | 53.506 | −11.180 | −19.736 | 1.00 | 55.16 | O |
| ATOM | 3045 | N | ASN | B | 288 | 51.265 | −10.941 | −19.641 | 1.00 | 61.80 | N |
| ATOM | 3046 | CA | ASN | B | 288 | 50.951 | −12.392 | −19.744 | 1.00 | 65.53 | C |
| ATOM | 3047 | CB | ASN | B | 288 | 49.508 | −12.662 | −19.279 | 1.00 | 67.92 | C |
| ATOM | 3048 | CG | ASN | B | 288 | 49.256 | −12.230 | −17.843 | 1.00 | 69.42 | C |
| ATOM | 3049 | OD1 | ASN | B | 288 | 50.169 | −12.247 | −17.015 | 1.00 | 69.19 | O |
| ATOM | 3050 | ND2 | ASN | B | 288 | 48.014 | −11.832 | −17.538 | 1.00 | 70.11 | N |
| ATOM | 3051 | C | ASN | B | 288 | 51.179 | −12.978 | −21.158 | 1.00 | 68.54 | C |
| ATOM | 3052 | O | ASN | B | 288 | 50.302 | −13.589 | −21.811 | 1.00 | 69.29 | O |
| TER | 3053 | | ASN | B | 288 | | | | | | |
| ATOM | 3054 | N | VAL | C | 97 | 15.048 | −15.040 | −16.506 | 1.00 | 55.54 | N |
| ATOM | 3055 | CA | VAL | C | 97 | 13.674 | −15.498 | −16.086 | 1.00 | 54.66 | C |
| ATOM | 3056 | CB | VAL | C | 97 | 12.686 | −14.309 | −16.079 | 1.00 | 55.66 | C |
| ATOM | 3057 | CG1 | VAL | C | 97 | 11.291 | −14.755 | −15.660 | 1.00 | 56.21 | C |
| ATOM | 3058 | CG2 | VAL | C | 97 | 13.185 | −13.184 | −15.176 | 1.00 | 55.91 | C |
| ATOM | 3059 | C | VAL | C | 97 | 13.174 | −16.556 | −17.078 | 1.00 | 52.41 | C |
| ATOM | 3060 | O | VAL | C | 97 | 12.976 | −16.232 | −18.250 | 1.00 | 52.11 | O |
| ATOM | 3061 | N | PRO | C | 98 | 13.062 | −17.829 | −16.649 | 1.00 | 51.91 | N |
| ATOM | 3062 | CA | PRO | C | 98 | 12.604 | −18.862 | −17.610 | 1.00 | 50.49 | C |
| ATOM | 3063 | CB | PRO | C | 98 | 12.789 | −20.194 | −16.873 | 1.00 | 50.63 | C |
| ATOM | 3064 | CG | PRO | C | 98 | 13.202 | −19.880 | −15.483 | 1.00 | 50.47 | C |
| ATOM | 3065 | CD | PRO | C | 98 | 13.479 | −18.406 | −15.364 | 1.00 | 50.86 | C |
| ATOM | 3066 | C | PRO | C | 98 | 11.152 | −18.634 | −17.982 | 1.00 | 48.50 | C |
| ATOM | 3067 | O | PRO | C | 98 | 10.370 | −18.227 | −17.124 | 1.00 | 50.61 | O |
| ATOM | 3068 | N | SER | C | 99 | 10.795 | −18.908 | −19.229 | 1.00 | 46.56 | N |
| ATOM | 3069 | CA | SER | C | 99 | 9.418 | −18.699 | −19.678 | 1.00 | 46.63 | C |
| ATOM | 3070 | CB | SER | C | 99 | 9.296 | −18.850 | −21.188 | 1.00 | 47.14 | C |
| ATOM | 3071 | OG | SER | C | 99 | 7.939 | −18.886 | −21.574 | 1.00 | 48.19 | O |
| ATOM | 3072 | C | SER | C | 99 | 8.480 | −19.682 | −18.984 | 1.00 | 46.96 | C |
| ATOM | 3073 | O | SER | C | 99 | 8.840 | −20.844 | −18.756 | 1.00 | 47.97 | O |
| ATOM | 3074 | N | GLN | C | 100 | 7.284 | −19.196 | −18.648 | 1.00 | 45.27 | N |
| ATOM | 3075 | CA | GLN | C | 100 | 6.228 | −20.031 | −18.090 | 1.00 | 44.78 | C |
| ATOM | 3076 | CB | GLN | C | 100 | 5.672 | −19.549 | −16.682 | 1.00 | 46.45 | C |
| ATOM | 3077 | CG | GLN | C | 100 | 5.171 | −18.201 | −16.576 | 1.00 | 48.21 | C |
| ATOM | 3078 | CD | GLN | C | 100 | 4.269 | −18.130 | −15.341 | 1.00 | 49.12 | C |
| ATOM | 3079 | OE1 | GLN | C | 100 | 4.762 | −18.049 | −14.222 | 1.00 | 49.30 | O |
| ATOM | 3080 | NE2 | GLN | C | 100 | 2.953 | −18.169 | −15.550 | 1.00 | 50.49 | N |
| ATOM | 3081 | C | GLN | C | 100 | 4.973 | −20.076 | −18.969 | 1.00 | 43.80 | C |
| ATOM | 3082 | O | GLN | C | 100 | 3.925 | −20.562 | −18.535 | 1.00 | 42.42 | O |
| ATOM | 3083 | N | LYS | C | 101 | 5.089 | −19.597 | −20.212 | 1.00 | 45.38 | N |
| ATOM | 3084 | CA | LYS | C | 101 | 3.951 | −19.516 | −21.122 | 1.00 | 45.24 | C |
| ATOM | 3085 | CB | LYS | C | 101 | 4.311 | −18.685 | −22.364 | 1.00 | 48.34 | C |
| ATOM | 3086 | CG | LYS | C | 101 | 3.200 | −18.495 | −23.391 | 1.00 | 51.60 | C |
| ATOM | 3087 | CD | LYS | C | 101 | 1.941 | −17.839 | −22.824 | 1.00 | 54.05 | C |
| ATOM | 3088 | CE | LYS | C | 101 | 0.965 | −17.412 | −23.911 | 1.00 | 55.88 | C |
| ATOM | 3089 | NZ | LYS | C | 101 | 1.468 | −16.250 | −24.691 | 1.00 | 55.90 | N |
| ATOM | 3090 | C | LYS | C | 101 | 3.538 | −20.924 | −21.526 | 1.00 | 42.94 | C |
| ATOM | 3091 | O | LYS | C | 101 | 4.368 | −21.725 | −21.962 | 1.00 | 42.00 | O |
| ATOM | 3092 | N | THR | C | 102 | 2.251 | −21.217 | −21.367 | 1.00 | 40.94 | N |
| ATOM | 3093 | CA | THR | C | 102 | 1.688 | −22.491 | −21.776 | 1.00 | 40.60 | C |
| ATOM | 3094 | CB | THR | C | 102 | 0.207 | −22.602 | −21.372 | 1.00 | 40.25 | C |
| ATOM | 3095 | OG1 | THR | C | 102 | 0.081 | −22.393 | −19.961 | 1.00 | 39.77 | O |
| ATOM | 3096 | CG2 | THR | C | 102 | −0.363 | −23.973 | −21.743 | 1.00 | 39.80 | C |
| ATOM | 3097 | C | THR | C | 102 | 1.832 | −22.693 | −23.286 | 1.00 | 41.36 | C |
| ATOM | 3098 | O | THR | C | 102 | 1.660 | −21.757 | −24.073 | 1.00 | 39.17 | O |
| ATOM | 3099 | N | TYR | C | 103 | 2.172 | −23.922 | −23.663 | 1.00 | 41.16 | N |
| ATOM | 3100 | CA | TYR | C | 103 | 2.450 | −24.251 | −25.056 | 1.00 | 42.03 | C |
| ATOM | 3101 | CB | TYR | C | 103 | 3.855 | −23.779 | −25.453 | 1.00 | 46.06 | C |
| ATOM | 3102 | CG | TYR | C | 103 | 3.995 | −23.450 | −26.924 | 1.00 | 50.35 | C |
| ATOM | 3103 | CD1 | TYR | C | 103 | 4.199 | −24.442 | −27.865 | 1.00 | 53.82 | C |
| ATOM | 3104 | CE1 | TYR | C | 103 | 4.334 | −24.158 | −29.218 | 1.00 | 55.67 | C |
| ATOM | 3105 | CZ | TYR | C | 103 | 4.359 | −22.829 | −29.636 | 1.00 | 57.08 | C |
| ATOM | 3106 | OH | TYR | C | 103 | 4.521 | −22.531 | −30.975 | 1.00 | 60.17 | O |
| ATOM | 3107 | CE2 | TYR | C | 103 | 4.213 | −21.801 | −28.710 | 1.00 | 56.68 | C |
| ATOM | 3108 | CD2 | TYR | C | 103 | 4.060 | −22.102 | −27.370 | 1.00 | 53.42 | C |
| ATOM | 3109 | C | TYR | C | 103 | 2.270 | −25.748 | −25.242 | 1.00 | 39.69 | C |
| ATOM | 3110 | O | TYR | C | 103 | 3.088 | −26.529 | −24.779 | 1.00 | 39.02 | O |
| ATOM | 3111 | N | GLN | C | 104 | 1.176 | −26.130 | −25.906 | 1.00 | 37.52 | N |
| ATOM | 3112 | CA | GLN | C | 104 | 0.874 | −27.538 | −26.142 | 1.00 | 35.96 | C |
| ATOM | 3113 | CB | GLN | C | 104 | −0.575 | −27.721 | −26.587 | 1.00 | 37.76 | C |
| ATOM | 3114 | CG | GLN | C | 104 | −1.604 | −27.300 | −25.548 | 1.00 | 39.73 | C |
| ATOM | 3115 | CD | GLN | C | 104 | −2.988 | −27.895 | −25.783 | 1.00 | 41.32 | C |
| ATOM | 3116 | OE1 | GLN | C | 104 | −3.261 | −28.522 | −26.818 | 1.00 | 45.19 | O |
| ATOM | 3117 | NE2 | GLN | C | 104 | −3.882 | −27.687 | −24.819 | 1.00 | 41.53 | N |
| ATOM | 3118 | C | GLN | C | 104 | 1.817 | −28.180 | −27.161 | 1.00 | 34.18 | C |
| ATOM | 3119 | O | GLN | C | 104 | 2.088 | −29.379 | −27.077 | 1.00 | 30.65 | O |
| ATOM | 3120 | N | GLY | C | 105 | 2.288 | −27.385 | −28.127 | 1.00 | 33.58 | N |
| ATOM | 3121 | CA | GLY | C | 105 | 3.244 | −27.835 | −29.126 | 1.00 | 32.70 | C |
| ATOM | 3122 | C | GLY | C | 105 | 2.704 | −28.837 | −30.121 | 1.00 | 31.06 | C |
| ATOM | 3123 | O | GLY | C | 105 | 1.498 | −29.056 | −30.188 | 1.00 | 33.59 | O |
| ATOM | 3124 | N | SER | C | 106 | 3.614 | −29.440 | −30.889 | 1.00 | 29.99 | N |
| ATOM | 3125 | CA | SER | C | 106 | 3.265 | −30.403 | −31.944 | 1.00 | 30.12 | C |
| ATOM | 3126 | CB | SER | C | 106 | 4.534 | −30.875 | −32.659 | 1.00 | 30.03 | C |
| ATOM | 3127 | OG | SER | C | 106 | 5.192 | −29.765 | −33.200 | 1.00 | 32.64 | O |
| ATOM | 3128 | C | SER | C | 106 | 2.521 | −31.632 | −31.450 | 1.00 | 27.66 | C |
| ATOM | 3129 | O | SER | C | 106 | 1.633 | −32.133 | −32.133 | 1.00 | 27.11 | O |
| ATOM | 3130 | N | TYR | C | 107 | 2.913 | −32.105 | −30.274 | 1.00 | 26.17 | N |
| ATOM | 3131 | CA | TYR | C | 107 | 2.391 | −33.359 | −29.714 | 1.00 | 25.96 | C |
| ATOM | 3132 | CB | TYR | C | 107 | 3.509 | −34.060 | −28.930 | 1.00 | 25.29 | C |
| ATOM | 3133 | CG | TYR | C | 107 | 4.700 | −34.347 | −29.819 | 1.00 | 24.77 | C |
| ATOM | 3134 | CD1 | TYR | C | 107 | 4.689 | −35.433 | −30.709 | 1.00 | 23.66 | C |
| ATOM | 3135 | CE1 | TYR | C | 107 | 5.748 | −35.674 | −31.556 | 1.00 | 23.38 | C |
| ATOM | 3136 | CZ | TYR | C | 107 | 6.847 | −34.833 | −31.535 | 1.00 | 24.22 | C |
| ATOM | 3137 | OH | TYR | C | 107 | 7.911 | −35.056 | −32.379 | 1.00 | 24.11 | O |
| ATOM | 3138 | CE2 | TYR | C | 107 | 6.879 | −33.735 | −30.677 | 1.00 | 24.56 | C |
| ATOM | 3139 | CD2 | TYR | C | 107 | 5.809 | −33.499 | −29.827 | 1.00 | 23.80 | C |
| ATOM | 3140 | C | TYR | C | 107 | 1.132 | −33.189 | −28.858 | 1.00 | 25.96 | C |
| ATOM | 3141 | O | TYR | C | 107 | 0.599 | −34.178 | −28.358 | 1.00 | 25.84 | O |
| ATOM | 3142 | N | GLY | C | 108 | 0.650 | −31.951 | −28.692 | 1.00 | 25.57 | N |
| ATOM | 3143 | CA | GLY | C | 108 | −0.539 | −31.689 | −27.877 | 1.00 | 25.68 | C |
| ATOM | 3144 | C | GLY | C | 108 | −0.324 | −32.010 | −26.404 | 1.00 | 25.70 | C |
| ATOM | 3145 | O | GLY | C | 108 | −1.112 | −32.732 | −25.797 | 1.00 | 24.61 | O |
| ATOM | 3146 | N | PHE | C | 109 | 0.763 | −31.473 | −25.849 | 1.00 | 26.56 | N |
| ATOM | 3147 | CA | PHE | C | 109 | 1.171 | −31.721 | −24.462 | 1.00 | 27.04 | C |
| ATOM | 3148 | CB | PHE | C | 109 | 2.650 | −31.357 | −24.290 | 1.00 | 26.77 | C |
| ATOM | 3149 | CG | PHE | C | 109 | 3.161 | −31.483 | −22.873 | 1.00 | 26.93 | C |
| ATOM | 3150 | CD1 | PHE | C | 109 | 3.075 | −32.688 | −22.190 | 1.00 | 26.79 | C |
| ATOM | 3151 | CE1 | PHE | C | 109 | 3.560 | −32.812 | −20.899 | 1.00 | 26.39 | C |
| ATOM | 3152 | CZ | PHE | C | 109 | 4.144 | −31.723 | −20.266 | 1.00 | 26.98 | C |
| ATOM | 3153 | CE2 | PHE | C | 109 | 4.231 | −30.505 | −20.934 | 1.00 | 26.75 | C |
| ATOM | 3154 | CD2 | PHE | C | 109 | 3.750 | −30.394 | −22.230 | 1.00 | 26.63 | C |
| ATOM | 3155 | C | PHE | C | 109 | 0.312 | −30.930 | −23.475 | 1.00 | 27.94 | C |
| ATOM | 3156 | O | PHE | C | 109 | 0.241 | −29.708 | −23.558 | 1.00 | 28.62 | O |
| ATOM | 3157 | N | ARG | C | 110 | −0.332 | −31.639 | −22.549 | 1.00 | 30.00 | N |
| ATOM | 3158 | CA | ARG | C | 110 | −1.150 | −31.032 | −21.486 | 1.00 | 30.91 | C |
| ATOM | 3159 | CB | ARG | C | 110 | −2.638 | −31.093 | −21.828 | 1.00 | 34.43 | C |
| ATOM | 3160 | CG | ARG | C | 110 | −3.014 | −30.714 | −23.264 | 1.00 | 38.57 | C |
| ATOM | 3161 | CD | ARG | C | 110 | −4.495 | −30.864 | −23.577 | 1.00 | 41.08 | C |
| ATOM | 3162 | NE | ARG | C | 110 | −5.077 | −32.161 | −23.227 | 1.00 | 43.57 | N |
| ATOM | 3163 | CZ | ARG | C | 110 | −4.819 | −33.328 | −23.828 | 1.00 | 46.64 | C |
| ATOM | 3164 | NH1 | ARG | C | 110 | −3.948 | −33.438 | −24.823 | 1.00 | 48.18 | N |
| ATOM | 3165 | NH2 | ARG | C | 110 | −5.451 | −34.423 | −23.400 | 1.00 | 47.09 | N |
| ATOM | 3166 | C | ARG | C | 110 | −0.899 | −31.798 | −20.175 | 1.00 | 28.88 | C |
| ATOM | 3167 | O | ARG | C | 110 | −0.421 | −32.936 | −20.194 | 1.00 | 27.05 | O |
| ATOM | 3168 | N | LEU | C | 111 | −1.216 | −31.163 | −19.049 | 1.00 | 27.82 | N |
| ATOM | 3169 | CA | LEU | C | 111 | −1.111 | −31.790 | −17.734 | 1.00 | 27.46 | C |
| ATOM | 3170 | CB | LEU | C | 111 | −0.507 | −30.829 | −16.707 | 1.00 | 26.39 | C |
| ATOM | 3171 | CG | LEU | C | 111 | 0.934 | −30.407 | −16.947 | 1.00 | 25.20 | C |
| ATOM | 3172 | CD1 | LEU | C | 111 | 1.340 | −29.408 | −15.880 | 1.00 | 25.40 | C |
| ATOM | 3173 | CD2 | LEU | C | 111 | 1.906 | −31.569 | −16.979 | 1.00 | 25.19 | C |
| ATOM | 3174 | C | LEU | C | 111 | −2.470 | −32.248 | −17.225 | 1.00 | 28.21 | C |
| ATOM | 3175 | O | LEU | C | 111 | −3.499 | −31.667 | −17.560 | 1.00 | 28.50 | O |
| ATOM | 3176 | N | GLY | C | 112 | −2.444 | −33.309 | −16.422 | 1.00 | 28.95 | N |
| ATOM | 3177 | CA | GLY | C | 112 | −3.619 | −33.817 | −15.726 | 1.00 | 29.21 | C |
| ATOM | 3178 | C | GLY | C | 112 | −3.277 | −34.085 | −14.278 | 1.00 | 29.01 | C |
| ATOM | 3179 | O | GLY | C | 112 | −2.097 | −34.234 | −13.914 | 1.00 | 29.95 | O |
| ATOM | 3180 | N | PHE | C | 113 | −4.311 | −34.131 | −13.444 | 1.00 | 28.79 | N |
| ATOM | 3181 | CA | PHE | C | 113 | −4.143 | −34.364 | −12.010 | 1.00 | 29.55 | C |
| ATOM | 3182 | CB | PHE | C | 113 | −4.213 | −33.040 | −11.256 | 1.00 | 28.85 | C |
| ATOM | 3183 | CG | PHE | C | 113 | −3.312 | −31.980 | −11.826 | 1.00 | 28.27 | C |
| ATOM | 3184 | CD1 | PHE | C | 113 | −1.990 | −31.897 | −11.439 | 1.00 | 28.39 | C |
| ATOM | 3185 | CE1 | PHE | C | 113 | −1.153 | −30.926 | −11.982 | 1.00 | 28.30 | C |
| ATOM | 3186 | CZ | PHE | C | 113 | −1.639 | −30.041 | −12.936 | 1.00 | 27.69 | C |
| ATOM | 3187 | CE2 | PHE | C | 113 | −2.952 | −30.125 | −13.336 | 1.00 | 27.66 | C |
| ATOM | 3188 | CD2 | PHE | C | 113 | −3.782 | −31.088 | −12.792 | 1.00 | 27.95 | C |
| ATOM | 3189 | C | PHE | C | 113 | −5.220 | −35.318 | −11.537 | 1.00 | 29.76 | C |
| ATOM | 3190 | O | PHE | C | 113 | −6.290 | −35.381 | −12.129 | 1.00 | 31.37 | O |
| ATOM | 3191 | N | LEU | C | 114 | −4.926 | −36.076 | −10.486 | 1.00 | 31.05 | N |
| ATOM | 3192 | CA | LEU | C | 114 | −5.924 | −36.951 | −9.882 | 1.00 | 32.03 | C |
| ATOM | 3193 | CB | LEU | C | 114 | −5.291 | −37.854 | −8.813 | 1.00 | 32.03 | C |
| ATOM | 3194 | CG | LEU | C | 114 | −4.287 | −38.914 | −9.229 | 1.00 | 32.32 | C |
| ATOM | 3195 | CD1 | LEU | C | 114 | −3.868 | −39.705 | −7.996 | 1.00 | 32.37 | C |
| ATOM | 3196 | CD2 | LEU | C | 114 | −4.870 | −39.867 | −10.255 | 1.00 | 32.52 | C |
| ATOM | 3197 | C | LEU | C | 114 | −7.033 | −36.103 | −9.261 | 1.00 | 32.26 | C |
| ATOM | 3198 | O | LEU | C | 114 | −6.794 | −34.973 | −8.828 | 1.00 | 32.18 | O |
| ATOM | 3199 | N | HIS | C | 115 | −8.230 | −36.664 | −9.229 | 1.00 | 32.59 | N |
| ATOM | 3200 | CA | HIS | C | 115 | −9.410 | −36.003 | −8.650 | 1.00 | 32.22 | C |
| ATOM | 3201 | CB | HIS | C | 115 | −10.654 | −36.252 | −9.505 | 1.00 | 33.82 | C |
| ATOM | 3202 | CG | HIS | C | 115 | −10.430 | −36.075 | −10.974 | 1.00 | 35.74 | C |
| ATOM | 3203 | ND1 | HIS | C | 115 | −10.377 | −37.138 | −11.858 | 1.00 | 35.94 | N |
| ATOM | 3204 | CE1 | HIS | C | 115 | −10.138 | −36.680 | −13.073 | 1.00 | 35.94 | C |
| ATOM | 3205 | NE2 | HIS | C | 115 | −10.034 | −35.361 | −13.010 | 1.00 | 36.48 | N |
| ATOM | 3206 | CD2 | HIS | C | 115 | −10.229 | −34.958 | −11.709 | 1.00 | 35.80 | C |
| ATOM | 3207 | C | HIS | C | 115 | −9.567 | −36.576 | −7.230 | 1.00 | 31.01 | C |
| ATOM | 3208 | O | HIS | O | 115 | −10.378 | −37.483 | −6.968 | 1.00 | 29.32 | O |
| ATOM | 3209 | N | SER | C | 116 | −8.765 | −36.029 | −6.318 | 1.00 | 30.44 | N |
| ATOM | 3210 | CA | SER | C | 116 | −8.537 | −36.653 | −5.014 | 1.00 | 31.27 | C |
| ATOM | 3211 | CB | SER | C | 116 | −7.086 | −36.467 | −4.590 | 1.00 | 31.40 | C |
| ATOM | 3212 | OG | SER | C | 116 | −6.205 | −36.912 | −5.617 | 1.00 | 32.97 | O |
| ATOM | 3213 | C | SER | C | 116 | −9.469 | −36.171 | −3.903 | 1.00 | 30.31 | C |
| ATOM | 3214 | O | SER | C | 116 | −9.593 | −36.830 | −2.876 | 1.00 | 30.49 | O |
| ATOM | 3215 | N | GLY | C | 117 | −10.126 | −35.029 | −4.111 | 1.00 | 30.08 | N |
| ATOM | 3216 | CA | GLY | C | 117 | −11.093 | −34.496 | −3.154 | 1.00 | 29.93 | C |
| ATOM | 3217 | C | GLY | C | 117 | −10.402 | −33.783 | −2.010 | 1.00 | 29.10 | C |
| ATOM | 3218 | O | GLY | C | 117 | −9.185 | −33.629 | −2.022 | 1.00 | 27.83 | O |
| ATOM | 3219 | N | THR | C | 118 | −11.185 | −33.350 | −1.020 | 1.00 | 28.53 | N |
| ATOM | 3220 | CA | THR | C | 118 | −10.666 | −32.528 | 0.074 | 1.00 | 29.23 | C |
| ATOM | 3221 | CB | THR | C | 118 | −11.241 | −31.090 | 0.012 | 1.00 | 27.41 | C |
| ATOM | 3222 | OG1 | THR | C | 118 | −12.673 | −31.124 | 0.079 | 1.00 | 26.62 | O |
| ATOM | 3223 | CG2 | THR | C | 118 | −10.782 | −30.397 | −1.267 | 1.00 | 26.78 | C |
| ATOM | 3224 | C | THR | C | 118 | −10.880 | −33.116 | 1.465 | 1.00 | 30.55 | C |
| ATOM | 3225 | O | THR | C | 118 | −10.900 | −32.378 | 2.456 | 1.00 | 31.04 | O |
| ATOM | 3226 | N | ALA | C | 119 | −10.993 | −34.439 | 1.555 | 1.00 | 31.56 | N |
| ATOM | 3227 | CA | ALA | C | 119 | −11.080 | −35.106 | 2.871 | 1.00 | 32.34 | C |
| ATOM | 3228 | CB | ALA | C | 119 | −11.337 | −36.596 | 2.713 | 1.00 | 31.79 | C |
| ATOM | 3229 | C | ALA | C | 119 | −9.797 | −34.858 | 3.675 | 1.00 | 34.19 | C |
| ATOM | 3230 | O | ALA | C | 119 | −8.705 | −34.774 | 3.103 | 1.00 | 33.81 | O |
| ATOM | 3231 | N | LYS | C | 120 | −9.947 | −34.749 | 4.992 | 1.00 | 36.89 | N |
| ATOM | 3232 | CA | LYS | C | 120 | −8.849 | −34.450 | 5.928 | 1.00 | 38.84 | C |
| ATOM | 3233 | CB | LYS | C | 120 | −9.333 | −34.538 | 7.389 | 1.00 | 42.28 | C |
| ATOM | 3234 | CG | LYS | C | 120 | −10.173 | −33.378 | 7.889 | 1.00 | 46.29 | C |
| ATOM | 3235 | CD | LYS | C | 120 | −10.654 | −33.677 | 9.310 | 1.00 | 49.27 | C |
| ATOM | 3236 | CE | LYS | C | 120 | −11.143 | −32.445 | 10.070 | 1.00 | 51.18 | C |
| ATOM | 3237 | NZ | LYS | C | 120 | −12.571 | −32.124 | 9.781 | 1.00 | 52.35 | N |
| ATOM | 3238 | C | LYS | C | 120 | −7.616 | −35.356 | 5.751 | 1.00 | 37.16 | C |
| ATOM | 3239 | O | LYS | C | 120 | −6.484 | −34.894 | 5.898 | 1.00 | 36.31 | O |
| ATOM | 3240 | N | SER | C | 121 | −7.846 | −36.627 | 5.410 | 1.00 | 36.47 | N |
| ATOM | 3241 | CA | SER | C | 121 | −6.767 | −37.604 | 5.235 | 1.00 | 37.19 | C |
| ATOM | 3242 | CB | SER | C | 121 | −7.328 | −39.019 | 5.404 | 1.00 | 36.89 | C |
| ATOM | 3243 | OG | SER | C | 121 | −6.201 | −39.337 | 4.334 | 1.00 | 37.82 | O |
| ATOM | 3244 | C | SER | C | 121 | −6.011 | −37.537 | 3.889 | 1.00 | 38.09 | C |
| ATOM | 3245 | O | SER | C | 121 | −5.113 | −38.367 | 3.646 | 1.00 | 39.01 | O |
| ATOM | 3246 | N | VAL | C | 122 | −6.346 | −36.582 | 3.020 | 1.00 | 36.90 | N |
| ATOM | 3247 | CA | VAL | C | 122 | −5.721 | −36.491 | 1.700 | 1.00 | 35.87 | C |
| ATOM | 3248 | CB | VAL | C | 122 | −6.638 | −35.732 | 0.691 | 1.00 | 35.03 | C |
| ATOM | 3249 | CG1 | VAL | C | 122 | −6.553 | −34.206 | 0.835 | 1.00 | 34.73 | C |
| ATOM | 3250 | CG2 | VAL | C | 122 | −6.321 | −36.151 | −0.736 | 1.00 | 33.98 | C |
| ATOM | 3251 | C | VAL | C | 122 | −4.310 | −35.878 | 1.822 | 1.00 | 35.54 | C |
| ATOM | 3252 | O | VAL | C | 122 | −4.129 | −34.840 | 2.475 | 1.00 | 37.08 | O |
| ATOM | 3253 | N | THR | C | 123 | −3.328 | −36.543 | 1.217 | 1.00 | 34.24 | N |
| ATOM | 3254 | CA | THR | C | 123 | −1.934 | −36.100 | 1.241 | 1.00 | 33.29 | C |
| ATOM | 3255 | CB | THR | C | 123 | −0.976 | −37.307 | 1.138 | 1.00 | 32.81 | C |
| ATOM | 3256 | OG1 | THR | C | 123 | −1.247 | −38.045 | −0.062 | 1.00 | 30.73 | O |
| ATOM | 3257 | CG2 | THR | C | 123 | −1.151 | 38.232 | 2.333 | 1.00 | 33.14 | C |
| ATOM | 3258 | C | THR | C | 123 | −1.595 | −35.129 | 0.104 | 1.00 | 33.31 | C |
| ATOM | 3259 | O | THR | C | 123 | −0.671 | −34.336 | 0.221 | 1.00 | 31.62 | O |
| ATOM | 3260 | N | CYS | C | 124 | −2.353 | −35.209 | −0.990 | 1.00 | 34.05 | N |
| ATOM | 3261 | CA | CYS | C | 124 | −2.143 | −34.417 | −2.186 | 1.00 | 34.49 | C |
| ATOM | 3262 | CB | CYS | C | 124 | −1.169 | −35.145 | −3.110 | 1.00 | 36.86 | C |
| ATOM | 3263 | SG | CYS | C | 124 | −0.734 | −34.234 | −4.598 | 1.00 | 43.10 | S |
| ATOM | 3264 | C | CYS | C | 124 | −3.481 | −34.201 | −2.909 | 1.00 | 32.64 | C |
| ATOM | 3265 | O | CYS | C | 124 | −4.221 | −35.157 | −3.138 | 1.00 | 30.99 | O |
| ATOM | 3266 | N | THR | C | 125 | −3.792 | −32.945 | −3.258 | 1.00 | 30.91 | N |
| ATOM | 3267 | CA | THR | C | 125 | −5.014 | −32.627 | −4.000 | 1.00 | 30.67 | C |
| ATOM | 3268 | CB | THR | C | 125 | −6.245 | −32.483 | −3.068 | 1.00 | 30.47 | C |
| ATOM | 3269 | OG1 | THR | C | 125 | −7.427 | −32.270 | −3.852 | 1.00 | 29.67 | O |
| ATOM | 3270 | CG2 | THR | C | 125 | −6.064 | −31.349 | −2.045 | 1.00 | 30.30 | C |
| ATOM | 3271 | C | THR | C | 125 | −4.860 | −31.368 | −4.853 | 1.00 | 30.77 | C |
| ATOM | 3272 | O | THR | C | 125 | −4.205 | −30.398 | −4.440 | 1.00 | 33.30 | O |
| ATOM | 3273 | N | TYR | C | 126 | −5.483 | −31.397 | −6.034 | 1.00 | 28.76 | N |
| ATOM | 3274 | CA | TYR | C | 126 | −5.386 | −30.327 | −7.022 | 1.00 | 27.41 | C |
| ATOM | 3275 | CB | TYR | C | 126 | −4.990 | −30.919 | −8.365 | 1.00 | 27.55 | C |
| ATOM | 3276 | CG | TYR | C | 126 | −5.015 | −29.941 | −9.517 | 1.00 | 27.66 | C |
| ATOM | 3277 | CD1 | TYR | C | 126 | −4.009 | −28.993 | −9.672 | 1.00 | 27.14 | C |
| ATOM | 3278 | CE1 | TYR | C | 126 | −4.026 | −28.103 | −10.731 | 1.00 | 27.68 | C |
| ATOM | 3279 | CZ | TYR | C | 126 | −5.066 | −28.156 | −11.658 | 1.00 | 27.50 | C |
| ATOM | 3280 | OH | TYR | C | 126 | −5.103 | −27.291 | −12.722 | 1.00 | 27.57 | O |
| ATOM | 3281 | CE2 | TYR | C | 126 | −6.073 | −29.093 | −11.524 | 1.00 | 27.72 | C |
| ATOM | 3282 | CD2 | TYR | C | 126 | −6.048 | −29.977 | −10.462 | 1.00 | 27.80 | C |
| ATOM | 3283 | C | TYR | C | 126 | −6.722 | −29.605 | −7.158 | 1.00 | 26.96 | C |
| ATOM | 3284 | O | TYR | C | 126 | −7.766 | −30.249 | −7.248 | 1.00 | 25.77 | O |
| ATOM | 3285 | N | SER | C | 127 | −6.664 | −28.272 | −7.181 | 1.00 | 27.23 | N |
| ATOM | 3286 | CA | SER | C | 127 | −7.829 | −27.422 | −7.381 | 1.00 | 27.52 | C |
| ATOM | 3287 | CB | SER | C | 127 | −7.737 | −26.192 | −6.484 | 1.00 | 28.00 | C |
| ATOM | 3288 | OG | SER | C | 127 | −8.667 | −25.183 | −6.887 | 1.00 | 28.07 | O |
| ATOM | 3289 | C | SER | C | 127 | −7.863 | −26.948 | −8.832 | 1.00 | 27.52 | C |
| ATOM | 3290 | O | SER | C | 127 | −6.987 | −26.173 | −9.231 | 1.00 | 28.75 | O |
| ATOM | 3291 | N | PRO | C | 128 | −8.848 | −27.421 | −9.628 | 1.00 | 27.69 | N |
| ATOM | 3292 | CA | PRO | C | 128 | −8.988 | −26.904 | −10.991 | 1.00 | 27.96 | C |
| ATOM | 3293 | CB | PRO | C | 128 | −10.169 | −27.706 | −11.551 | 1.00 | 27.56 | C |
| ATOM | 3294 | CG | PRO | C | 128 | −10.144 | −28.982 | −10.803 | 1.00 | 27.73 | C |
| ATOM | 3295 | CD | PRO | C | 128 | −9.660 | −28.634 | −9.423 | 1.00 | 27.90 | C |
| ATOM | 3296 | C | PRO | C | 128 | −9.265 | −25.405 | −11.078 | 1.00 | 28.27 | C |
| ATOM | 3297 | O | PRO | C | 128 | −8.740 | −24.740 | −11.961 | 1.00 | 26.47 | O |
| ATOM | 3298 | N | ALA | C | 129 | −10.073 | −24.881 | −10.160 | 1.00 | 30.18 | N |
| ATOM | 3299 | CA | ALA | C | 129 | −10.449 | −23.464 | −10.189 | 1.00 | 30.93 | C |
| ATOM | 3300 | CB | ALA | C | 129 | −11.520 | −23.176 | −9.147 | 1.00 | 31.28 | C |
| ATOM | 3301 | C | ALA | C | 129 | −9.250 | −22.545 | −9.988 | 1.00 | 30.63 | C |
| ATOM | 3302 | O | ALA | C | 129 | −9.163 | −21.496 | −10.621 | 1.00 | 32.30 | O |
| ATOM | 3303 | N | LEU | C | 130 | −8.331 | −22.948 | −9.115 | 1.00 | 30.85 | N |
| ATOM | 3304 | CA | LEU | C | 130 | −7.131 | −22.177 | −8.808 | 1.00 | 30.27 | C |
| ATOM | 3305 | CB | LEU | C | 130 | −6.837 | −22.277 | −7.308 | 1.00 | 30.29 | C |
| ATOM | 3306 | CG | LEU | C | 130 | −7.831 | −21.590 | −6.365 | 1.00 | 31.72 | C |
| ATOM | 3307 | CD1 | LEU | C | 130 | −7.725 | −22.129 | −4.945 | 1.00 | 32.00 | C |
| ATOM | 3308 | CD2 | LEU | C | 130 | −7.593 | −20.084 | −6.366 | 1.00 | 32.27 | C |
| ATOM | 3309 | C | LEU | C | 130 | −5.881 | −22.604 | −9.602 | 1.00 | 30.22 | C |
| ATOM | 3310 | O | LEU | C | 130 | −4.863 | −21.908 | −9.543 | 1.00 | 30.51 | O |
| ATOM | 3311 | N | ASN | C | 131 | −5.955 | −23.733 | −10.326 | 1.00 | 29.13 | N |
| ATOM | 3312 | CA | ASN | C | 131 | −4.777 | −24.356 | −10.950 | 1.00 | 28.20 | C |
| ATOM | 3313 | CB | ASN | C | 131 | −4.330 | −23.601 | −12.218 | 1.00 | 27.25 | C |
| ATOM | 3314 | CG | ASN | C | 131 | −3.247 | −24.357 | −13.012 | 1.00 | 26.78 | C |
| ATOM | 3315 | OD1 | ASN | C | 131 | −3.177 | −25.586 | −12.993 | 1.00 | 26.48 | O |
| ATOM | 3316 | ND2 | ASN | C | 131 | −2.397 | −23.622 | −13.702 | 1.00 | 25.12 | N |
| ATOM | 3317 | C | ASN | C | 131 | −3.640 | −24.470 | −9.926 | 1.00 | 29.31 | C |
| ATOM | 3318 | O | ASN | C | 131 | −2.497 | −24.045 | −10.164 | 1.00 | 28.38 | O |
| ATOM | 3319 | N | LYS | C | 132 | −3.988 | −25.039 | −8.771 | 1.00 | 29.92 | N |
| ATOM | 3320 | CA | LYS | C | 132 | −3.110 | −25.056 | −7.614 | 1.00 | 30.24 | C |
| ATOM | 3321 | CB | LYS | C | 132 | −3.567 | −24.011 | −6.606 | 1.00 | 29.21 | C |
| ATOM | 3322 | CG | LYS | C | 132 | −2.594 | −23.784 | −5.458 | 1.00 | 29.41 | C |
| ATOM | 3323 | CD | LYS | C | 132 | −2.999 | −22.542 | −4.693 | 1.00 | 29.54 | C |
| ATOM | 3324 | CE | LYS | C | 132 | −2.157 | −22.312 | −3.452 | 1.00 | 28.95 | C |
| ATOM | 3325 | NZ | LYS | C | 132 | −2.458 | −20.979 | −2.854 | 1.00 | 28.73 | N |
| ATOM | 3326 | C | LYS | C | 132 | −3.090 | −26.436 | −6.961 | 1.00 | 30.42 | C |
| ATOM | 3327 | O | LYS | C | 132 | −4.137 | −27.000 | −6.635 | 1.00 | 29.27 | O |
| ATOM | 3328 | N | MET | C | 133 | −1.884 | −26.959 | −6.778 | 1.00 | 31.65 | N |
| ATOM | 3329 | CA | MET | C | 133 | −1.676 | −28.218 | −6.089 | 1.00 | 33.28 | C |
| ATOM | 3330 | CB | MET | C | 133 | −0.476 | −28.927 | −6.705 | 1.00 | 34.58 | C |
| ATOM | 3331 | CG | MET | C | 133 | −0.164 | −30.276 | −6.102 | 1.00 | 36.58 | C |
| ATOM | 3332 | SD | MET | C | 133 | −1.358 | −31.541 | −6.522 | 1.00 | 39.10 | S |
| ATOM | 3333 | CE | MET | C | 133 | −0.817 | −31.889 | −8.196 | 1.00 | 38.67 | C |
| ATOM | 3334 | C | MET | C | 133 | −1.438 | −27.937 | −4.604 | 1.00 | 32.59 | C |
| ATOM | 3335 | O | MET | C | 133 | −0.675 | −27.033 | −4.258 | 1.00 | 31.37 | O |
| ATOM | 3336 | N | PHE | C | 134 | −2.107 | −28.705 | −3.416 | 1.00 | 32.62 | N |
| ATOM | 3337 | CA | PHE | C | 134 | −1.888 | −28.660 | −2.302 | 1.00 | 34.36 | C |
| ATOM | 3338 | CB | PHE | C | 134 | −3.207 | −28.404 | −1.568 | 1.00 | 33.63 | C |
| ATOM | 3339 | CG | PHE | C | 134 | −3.829 | −27.076 | −1.887 | 1.00 | 33.94 | C |
| ATOM | 3340 | CD1 | PHE | C | 134 | −4.551 | −26.897 | −3.063 | 1.00 | 33.75 | C |
| ATOM | 3341 | CE1 | PHE | C | 134 | −5.142 | −25.678 | −3.364 | 1.00 | 33.15 | C |
| ATOM | 3342 | CZ | PHE | C | 134 | −5.017 | −24.616 | −2.481 | 1.00 | 34.27 | C |
| ATOM | 3343 | CE2 | PHE | C | 134 | −4.309 | −24.776 | −1.290 | 1.00 | 34.02 | C |
| ATOM | 3344 | CD2 | PHE | C | 134 | −3.718 | −26.003 | −1.003 | 1.00 | 34.50 | C |
| ATOM | 3345 | C | PHE | C | 134 | −1.323 | −30.018 | −1.894 | 1.00 | 34.97 | C |
| ATOM | 3346 | O | PHE | C | 134 | −1.954 | −31.041 | −2.153 | 1.00 | 35.68 | O |
| ATOM | 3347 | N | CYS | C | 135 | −0.144 | −30.023 | −1.277 | 1.00 | 37.07 | N |
| ATOM | 3348 | CA | CYS | C | 135 | 0.512 | −31.274 | −0.862 | 1.00 | 40.71 | C |
| ATOM | 3349 | CB | CYS | C | 135 | 1.620 | −31.687 | −1.853 | 1.00 | 43.60 | C |
| ATOM | 3350 | SG | CYS | C | 135 | 1.701 | −30.945 | −3.517 | 1.00 | 49.76 | S |
| ATOM | 3351 | C | CYS | C | 135 | 1.163 | −31.155 | 0.513 | 1.00 | 40.41 | C |
| ATOM | 3352 | O | CYS | C | 135 | 1.506 | −30.069 | 0.967 | 1.00 | 38.01 | O |
| ATOM | 3353 | N | GLN | C | 136 | 1.340 | −32.304 | 1.148 | 1.00 | 41.43 | N |
| ATOM | 3354 | CA | GLN | C | 136 | 2.123 | −32.410 | 2.375 | 1.00 | 44.37 | C |
| ATOM | 3355 | CB | GLN | C | 136 | 1.639 | −33.582 | 3.233 | 1.00 | 45.69 | C |
| ATOM | 3356 | CG | GLN | C | 136 | 0.357 | −33.308 | 3.979 | 1.00 | 47.77 | C |
| ATOM | 3357 | CD | GLN | C | 136 | −0.187 | −34.513 | 4.734 | 1.00 | 49.87 | C |
| ATOM | 3358 | OE1 | GLN | C | 136 | 0.527 | −35.494 | 4.990 | 1.00 | 50.73 | O |
| ATOM | 3359 | NE2 | GLN | C | 136 | −1.469 | −34.462 | 5.067 | 1.00 | 51.61 | N |
| ATOM | 3360 | C | GLN | C | 136 | 3.602 | −32.561 | 2.009 | 1.00 | 45.33 | C |
| ATOM | 3361 | O | GLN | C | 136 | 3.953 | −33.086 | 0.943 | 1.00 | 45.34 | O |
| ATOM | 3362 | N | LEU | C | 137 | 4.459 | −32.084 | 2.912 | 1.00 | 45.91 | N |
| ATOM | 3363 | CA | LEU | C | 137 | 5.911 | −32.089 | 2.714 | 1.00 | 46.78 | C |
| ATOM | 3364 | CB | LEU | C | 137 | 6.597 | −31.417 | 3.913 | 1.00 | 46.74 | C |
| ATOM | 3365 | CG | LEU | C | 137 | 8.061 | −31.674 | 4.298 | 1.00 | 47.26 | C |
| ATOM | 3366 | CD1 | LEU | C | 137 | 9.041 | −30.945 | 3.419 | 1.00 | 47.79 | C |
| ATOM | 3367 | CD2 | LEU | C | 137 | 8.276 | −31.263 | 5.744 | 1.00 | 47.88 | C |
| ATOM | 3368 | C | LEU | C | 137 | 6.432 | −33.525 | 2.521 | 1.00 | 46.32 | C |
| ATOM | 3369 | O | LEU | C | 137 | 6.092 | 34.435 | 3.288 | 1.00 | 46.12 | O |
| ATOM | 3370 | N | ALA | C | 138 | 7.235 | −33.699 | 1.476 | 1.00 | 45.49 | N |
| ATOM | 3371 | CA | ALA | C | 138 | 7.863 | −34.982 | 1.124 | 1.00 | 46.09 | C |
| ATOM | 3372 | CB | ALA | C | 138 | 8.867 | −35.372 | 2.196 | 1.00 | 46.53 | C |
| ATOM | 3373 | C | ALA | C | 138 | 6.902 | −36.154 | 0.827 | 1.00 | 45.52 | C |
| ATOM | 3374 | O | ALA | C | 138 | 7.308 | −37.311 | 0.904 | 1.00 | 46.42 | O |
| ATOM | 3375 | N | LYS | C | 139 | 5.656 | −35.853 | 0.461 | 1.00 | 43.63 | N |
| ATOM | 3376 | CA | LYS | C | 139 | 4.690 | −36.889 | 0.096 | 1.00 | 41.76 | C |
| ATOM | 3377 | CB | LYS | C | 139 | 3.321 | −36.607 | 0.701 | 1.00 | 43.15 | C |
| ATOM | 3378 | CG | LYS | C | 139 | 3.288 | −36.572 | 2.211 | 1.00 | 45.11 | C |
| ATOM | 3379 | CD | LYS | C | 139 | 3.359 | −37.935 | 2.858 | 1.00 | 46.03 | C |
| ATOM | 3380 | CE | LYS | C | 139 | 3.215 | −37.778 | 4.378 | 1.00 | 45.75 | C |
| ATOM | 3381 | NZ | LYS | C | 139 | 2.600 | −38.941 | 5.099 | 1.00 | 45.97 | N |
| ATOM | 3382 | C | LYS | C | 139 | 4.564 | −36.965 | −1.425 | 1.00 | 38.26 | C |
| ATOM | 3383 | O | LYS | C | 139 | 4.826 | −35.989 | −2.134 | 1.00 | 35.00 | O |
| ATOM | 3384 | N | THR | C | 140 | 4.161 | −38.139 | −1.897 | 1.00 | 36.12 | N |
| ATOM | 3385 | CA | THR | C | 140 | 3.899 | −38.389 | −3.309 | 1.00 | 36.60 | C |
| ATOM | 3386 | CB | THR | C | 140 | 3.312 | −39.797 | −3.520 | 1.00 | 36.30 | C |
| ATOM | 3387 | OG1 | THR | C | 140 | 4.139 | −40.761 | −2.862 | 1.00 | 37.15 | O |
| ATOM | 3388 | CG2 | THR | C | 140 | 3.223 | −40.144 | −4.992 | 1.00 | 36.76 | C |
| ATOM | 3389 | C | THR | C | 140 | 2.921 | −37.367 | −3.890 | 1.00 | 36.25 | C |
| ATOM | 3390 | O | THR | C | 140 | 1.872 | −37.110 | −3.311 | 1.00 | 36.03 | O |
| ATOM | 3391 | N | CYS | C | 141 | 3.295 | −36.788 | −5.022 | 1.00 | 36.74 | N |
| ATOM | 3392 | CA | CYS | C | 141 | 2.493 | −35.781 | −5.701 | 1.00 | 36.39 | C |
| ATOM | 3393 | CB | CYS | C | 141 | 3.111 | −34.394 | −5.493 | 1.00 | 38.17 | C |
| ATOM | 3394 | SG | CYS | C | 141 | 2.018 | −33.024 | −5.960 | 1.00 | 43.76 | S |
| ATOM | 3395 | C | CYS | C | 141 | 2.429 | −36.173 | −7.179 | 1.00 | 33.24 | C |
| ATOM | 3396 | O | CYS | C | 141 | 3.355 | −35.872 | −7.935 | 1.00 | 31.58 | O |
| ATOM | 3397 | N | PRO | C | 142 | 1.367 | −36.905 | −7.581 | 1.00 | 31.14 | N |
| ATOM | 3398 | CA | PRO | C | 142 | 1.292 | −37.345 | −8.973 | 1.00 | 29.96 | C |
| ATOM | 3399 | CB | PRO | C | 142 | 0.162 | −38.397 | −8.970 | 1.00 | 29.91 | C |
| ATOM | 3400 | CG | PRO | C | 142 | −0.301 | −38.530 | −7.568 | 1.00 | 30.19 | C |
| ATOM | 3401 | CD | PRO | C | 142 | 0.190 | −37.350 | −6.812 | 1.00 | 30.79 | C |
| ATOM | 3402 | C | PRO | C | 142 | 0.964 | −36.214 | −9.932 | 1.00 | 28.66 | C |
| ATOM | 3403 | O | PRO | C | 142 | 0.073 | −35.421 | −9.654 | 1.00 | 28.97 | O |
| ATOM | 3404 | N | VAL | C | 143 | 1.705 | −36.136 | −11.035 | 1.00 | 27.48 | N |
| ATOM | 3405 | CA | VAL | C | 143 | 1.389 | −35.230 | −12.146 | 1.00 | 26.65 | C |
| ATOM | 3406 | CB | VAL | C | 143 | 2.445 | −34.108 | −12.283 | 1.00 | 25.72 | C |
| ATOM | 3407 | CG1 | VAL | C | 143 | 2.184 | −33.269 | −13.544 | 1.00 | 25.26 | C |
| ATOM | 3408 | CG2 | VAL | C | 143 | 2.447 | −33.240 | −11.028 | 1.00 | 24.57 | C |
| ATOM | 3409 | C | VAL | C | 143 | 1.315 | −36.077 | −13.411 | 1.00 | 26.43 | C |
| ATOM | 3410 | O | VAL | C | 143 | 2.223 | −36.874 | −13.675 | 1.00 | 27.45 | O |
| ATOM | 3411 | N | GLN | C | 144 | 0.235 | −35.899 | −14.171 | 1.00 | 25.48 | N |
| ATOM | 3412 | CA | GLN | C | 144 | −0.022 | −36.686 | −15.378 | 1.00 | 24.91 | C |
| ATOM | 3413 | CB | GLN | C | 144 | −1.508 | −37.045 | −15.495 | 1.00 | 24.53 | C |
| ATOM | 3414 | CG | GLN | C | 144 | −2.062 | −37.818 | −14.304 | 1.00 | 24.12 | C |
| ATOM | 3415 | CD | GLN | C | 144 | −3.576 | −38.019 | −14.383 | 1.00 | 24.78 | C |
| ATOM | 3416 | OE1 | GLN | C | 144 | −4.284 | −37.319 | −15.123 | 1.00 | 24.62 | O |
| ATOM | 3417 | NE2 | GLN | C | 144 | −4.079 | −38.976 | −13.621 | 1.00 | 24.13 | N |
| ATOM | 3418 | C | GLN | C | 144 | 0.387 | −35.900 | −16.621 | 1.00 | 24.45 | C |
| ATOM | 3419 | O | GLN | C | 144 | 0.156 | −34.697 | −16.703 | 1.00 | 23.54 | O |
| ATOM | 3420 | N | LEU | C | 145 | 0.978 | −36.604 | −17.579 | 1.00 | 24.34 | N |
| ATOM | 3421 | CA | LEU | C | 145 | 1.335 | −36.061 | −18.877 | 1.00 | 24.09 | C |
| ATOM | 3422 | CB | LEU | C | 145 | 2.772 | −36.448 | −19.231 | 1.00 | 23.82 | C |
| ATOM | 3423 | CG | LEU | C | 145 | 3.809 | −36.323 | −18.112 | 1.00 | 23.81 | C |
| ATOM | 3424 | CD1 | LEU | C | 145 | 5.153 | −36.804 | −18.619 | 1.00 | 23.63 | C |
| ATOM | 3425 | CD2 | LEU | C | 145 | 3.872 | −34.911 | −17.544 | 1.00 | 23.89 | C |
| ATOM | 3426 | C | LEU | C | 145 | 0.357 | −36.620 | −19.910 | 1.00 | 24.80 | C |
| ATOM | 3427 | O | LEU | C | 145 | 0.265 | −37.828 | −20.085 | 1.00 | 24.82 | O |
| ATOM | 3428 | N | TRP | C | 146 | −0.382 | −35.733 | −20.563 | 1.00 | 25.69 | N |
| ATOM | 3429 | CA | TRP | C | 146 | −1.340 | −36.094 | −21.599 | 1.00 | 26.48 | C |
| ATOM | 3430 | CB | TRP | C | 146 | −2.719 | −35.509 | −21.280 | 1.00 | 26.95 | C |
| ATOM | 3431 | CG | TRP | C | 146 | −3.411 | −36.139 | −20.098 | 1.00 | 27.92 | C |
| ATOM | 3432 | CD1 | TRP | C | 146 | −3.121 | −35.944 | −18.784 | 1.00 | 28.46 | C |
| ATOM | 3433 | NE1 | TRP | C | 146 | −3.970 | −36.676 | −17.988 | 1.00 | 28.88 | N |
| ATOM | 3434 | CE2 | TRP | C | 146 | −4.842 | −37.367 | −18.790 | 1.00 | 29.48 | C |
| ATOM | 3435 | CD2 | TRP | C | 146 | −4.520 | −37.052 | −20.132 | 1.00 | 29.24 | C |
| ATOM | 3436 | CE3 | TRP | C | 146 | −5.256 | −37.656 | −21.165 | 1.00 | 29.79 | C |
| ATOM | 3437 | CZ3 | TRP | C | 146 | −6.310 | −38.519 | −20.819 | 1.00 | 29.80 | C |
| ATOM | 3438 | CH2 | TRP | C | 146 | −6.598 | −38.811 | −19.473 | 1.00 | 28.97 | C |
| ATOM | 3439 | CZ2 | TRP | C | 146 | −5.883 | −38.245 | −18.450 | 1.00 | 28.69 | C |
| ATOM | 3440 | C | TRP | C | 146 | −0.846 | −35.544 | −22.928 | 1.00 | 27.16 | C |
| ATOM | 3441 | O | TRP | C | 146 | −0.509 | −34.366 | −23.017 | 1.00 | 26.72 | O |
| ATOM | 3442 | N | VAL | C | 147 | −0.782 | −36.409 | −23.942 | 1.00 | 27.99 | N |
| ATOM | 3443 | CA | VAL | C | 147 | −0.388 | −36.018 | −25.306 | 1.00 | 29.07 | C |
| ATOM | 3444 | CB | VAL | C | 147 | 1.044 | −36.500 | −25.666 | 1.00 | 29.31 | C |
| ATOM | 3445 | CG1 | VAL | C | 147 | 2.077 | −35.735 | −24.857 | 1.00 | 29.26 | C |
| ATOM | 3446 | CG2 | VAL | C | 147 | 1.214 | −38.015 | −25.467 | 1.00 | 29.46 | C |
| ATOM | 3447 | C | VAL | C | 147 | −1.374 | −36.541 | −26.348 | 1.00 | 29.75 | C |
| ATOM | 3448 | O | VAL | C | 147 | −1.962 | −37.607 | −26.178 | 1.00 | 30.13 | O |
| ATOM | 3449 | N | ASP | C | 148 | −1.556 | −35.770 | −27.418 | 1.00 | 31.36 | N |
| ATOM | 3450 | CA | ASP | C | 148 | −2.325 | −36.216 | −28.593 | 1.00 | 32.71 | C |
| ATOM | 3451 | CB | ASP | C | 148 | −2.749 | −35.031 | −29.470 | 1.00 | 33.06 | C |
| ATOM | 3452 | CG | ASP | C | 148 | −3.682 | −34.059 | −28.751 | 1.00 | 34.24 | C |
| ATOM | 3453 | OD1 | ASP | C | 148 | −4.387 | −34.459 | −27.791 | 1.00 | 35.65 | O |
| ATOM | 3454 | OD1 | ASP | C | 148 | −3.702 | −32.874 | −29.156 | 1.00 | 34.21 | O |
| ATOM | 3455 | C | ASP | C | 148 | −1.506 | −37.190 | −29.443 | 1.00 | 32.86 | C |
| ATOM | 3456 | O | ASP | C | 148 | −2.064 | −38.131 | −29.998 | 1.00 | 35.10 | O |
| ATOM | 3457 | N | SER | C | 149 | −0.194 | −36.952 | −29.546 | 1.00 | 32.12 | N |
| ATOM | 3458 | CA | SER | C | 149 | 0.708 | −37.783 | −30.347 | 1.00 | 31.25 | C |
| ATOM | 3459 | CB | SER | C | 149 | 1.157 | −37.024 | −31.597 | 1.00 | 30.86 | C |
| ATOM | 3460 | OG | SER | C | 149 | 0.068 | −36.375 | −32.232 | 1.00 | 33.23 | O |
| ATOM | 3461 | C | SER | C | 149 | 1.933 | −38.156 | −29.510 | 1.00 | 29.88 | C |
| ATOM | 3462 | O | SER | C | 149 | 2.552 | −37.296 | −28.888 | 1.00 | 29.25 | O |
| ATOM | 3463 | N | THR | C | 150 | 2.282 | −39.444 | −29.514 | 1.00 | 28.03 | N |
| ATOM | 3464 | CA | THR | C | 150 | 3.456 | −39.934 | −28.814 | 1.00 | 27.95 | C |
| ATOM | 3465 | CB | THR | C | 150 | 3.553 | −41.469 | −28.919 | 1.00 | 28.20 | C |
| ATOM | 3466 | OG1 | THR | C | 150 | 2.351 | −42.029 | −28.376 | 1.00 | 28.84 | O |
| ATOM | 3467 | CG2 | THR | C | 150 | 4.739 | −42.019 | −28.115 | 1.00 | 28.10 | C |
| ATOM | 3468 | C | THR | C | 150 | 4.735 | −39.282 | −29.374 | 1.00 | 27.12 | C |
| ATOM | 3469 | O | THR | C | 150 | 4.990 | −39.361 | −30.573 | 1.00 | 26.62 | O |
| ATOM | 3470 | N | PRO | C | 151 | 5.526 | −38.612 | −28.506 | 1.00 | 26.71 | N |
| ATOM | 3471 | CA | PRO | C | 151 | 6.793 | −38.055 | −28.981 | 1.00 | 25.79 | C |
| ATOM | 3472 | CB | PRO | C | 151 | 7.176 | −37.039 | −27.900 | 1.00 | 26.31 | C |
| ATOM | 3473 | CG | PRO | C | 151 | 6.329 | 37.326 | −26.707 | 1.00 | 25.95 | C |
| ATOM | 3474 | CD | PRO | C | 151 | 5.257 | −38.280 | −27.093 | 1.00 | 26.50 | C |
| ATOM | 3475 | C | PRO | C | 151 | 7.868 | −39.137 | −29.172 | 1.00 | 25.67 | C |
| ATOM | 3476 | O | PRO | C | 151 | 7.727 | −40.241 | −28.641 | 1.00 | 26.23 | O |
| ATOM | 3477 | N | PRO | C | 152 | 8.940 | −38.817 | −29.926 | 1.00 | 24.64 | N |
| ATOM | 3478 | CA | PRO | C | 152 | 9.939 | −39.829 | −30.271 | 1.00 | 24.50 | C |
| ATOM | 3479 | CB | PRO | C | 152 | 10.885 | −39.106 | −31.243 | 1.00 | 24.67 | C |
| ATOM | 3480 | CG | PRO | C | 152 | 10.538 | −37.686 | −31.222 | 1.00 | 24.48 | C |
| ATOM | 3481 | CD | PRO | C | 152 | 9.255 | −37.486 | −30.479 | 1.00 | 24.45 | C |
| ATOM | 3482 | C | PRO | C | 152 | 10.731 | −40.354 | −29.059 | 1.00 | 25.32 | C |
| ATOM | 3483 | O | PRO | C | 152 | 10.787 | −39.668 | −28.028 | 1.00 | 23.04 | O |
| ATOM | 3484 | N | PRO | C | 153 | 11.355 | −41.549 | −29.186 | 1.00 | 26.64 | N |
| ATOM | 3485 | CA | PRO | C | 153 | 12.240 | −42.058 | −28.136 | 1.00 | 26.11 | C |
| ATOM | 3486 | CB | PRO | C | 153 | 12.862 | −43.317 | −28.769 | 1.00 | 26.77 | C |
| ATOM | 3487 | CG | PRO | C | 153 | 11.921 | −43.730 | −29.851 | 1.00 | 26.66 | C |
| ATOM | 3488 | CD | PRO | C | 153 | 11.248 | −42.492 | −30.324 | 1.00 | 26.45 | C |
| ATOM | 3489 | C | PRO | C | 153 | 13.339 | −41.066 | −27.742 | 1.00 | 24.74 | C |
| ATOM | 3490 | O | PRO | C | 153 | 13.777 | −40.274 | −28.582 | 1.00 | 24.17 | O |
| ATOM | 3491 | N | GLY | C | 154 | 13.748 | −41.107 | −26.471 | 1.00 | 22.82 | N |
| ATOM | 3492 | CA | GLY | C | 154 | 14.722 | −40.166 | −25.926 | 1.00 | 21.66 | C |
| ATOM | 3493 | C | GLY | C | 154 | 14.144 | −38.847 | −25.445 | 1.00 | 21.16 | C |
| ATOM | 3494 | O | GLY | C | 154 | 14.899 | −37.955 | −25.032 | 1.00 | 20.72 | O |
| ATOM | 3495 | N | THR | C | 155 | 12.820 | −38.686 | −25.556 | 1.00 | 20.71 | N |
| ATOM | 3496 | CA | THR | C | 155 | 12.115 | −37.511 | −25.032 | 1.00 | 19.93 | C |
| ATOM | 3497 | CB | THR | C | 155 | 10.626 | −37.548 | −25.436 | 1.00 | 19.66 | C |
| ATOM | 3498 | OG1 | THR | C | 155 | 10.527 | −37.423 | −26.856 | 1.00 | 19.69 | O |
| ATOM | 3499 | CG2 | THR | C | 155 | 9.811 | −36.409 | −24.788 | 1.00 | 19.64 | C |
| ATOM | 3500 | C | THR | C | 155 | 12.254 | −37.475 | −23.512 | 1.00 | 20.29 | C |
| ATOM | 3501 | O | THR | C | 155 | 12.276 | −38.528 | −22.859 | 1.00 | 20.66 | O |
| ATOM | 3502 | N | ARG | C | 156 | 12.343 | −36.267 | −22.962 | 1.00 | 20.47 | N |
| ATOM | 3503 | CA | ARG | C | 156 | 12.549 | −36.054 | −21.527 | 1.00 | 19.77 | C |
| ATOM | 3504 | CB | ARG | C | 156 | 13.979 | −35.563 | −21.282 | 1.00 | 19.82 | C |
| ATOM | 3505 | CG | ARG | C | 156 | 14.984 | −36.690 | −21.042 | 1.00 | 20.44 | C |
| ATOM | 3506 | CD | ARG | C | 156 | 16.342 | −36.462 | −21.686 | 1.00 | 20.62 | C |
| ATOM | 3507 | NE | ARG | C | 156 | 17.082 | −35.318 | −21.140 | 1.00 | 21.02 | N |
| ATOM | 3508 | CZ | ARG | C | 156 | 17.964 | −35.351 | −20.136 | 1.00 | 21.15 | C |
| ATOM | 3509 | NH1 | ARG | C | 156 | 18.245 | −36.485 | −19.484 | 1.00 | 21.34 | N |
| ATOM | 3510 | NH2 | ARG | C | 156 | 18.568 | −34.221 | −19.767 | 1.00 | 20.47 | N |
| ATOM | 3511 | C | ARG | C | 156 | 11.533 | −35.071 | −20.945 | 1.00 | 19.27 | C |
| ATOM | 3512 | O | ARG | C | 156 | 10.975 | −34.231 | −21.663 | 1.00 | 18.87 | O |
| ATOM | 3513 | N | VAL | C | 157 | 11.305 | −35.194 | −19.632 | 1.00 | 19.05 | N |
| ATOM | 3514 | CA | VAL | C | 157 | 10.336 | −34.390 | −18.894 | 1.00 | 18.25 | C |
| ATOM | 3515 | CB | VAL | C | 157 | 9.199 | −35.274 | −18.341 | 1.00 | 17.81 | C |
| ATOM | 3516 | CG1 | VAL | C | 157 | 8.043 | −34.412 | −17.830 | 1.00 | 17.58 | C |
| ATOM | 3517 | CG2 | VAL | C | 157 | 8.698 | −36.253 | −19.403 | 1.00 | 17.83 | C |
| ATOM | 3518 | C | VAL | C | 157 | 11.041 | −33.687 | −17.735 | 1.00 | 18.20 | C |
| ATOM | 3519 | O | VAL | C | 157 | 11.550 | −34.345 | −16.825 | 1.00 | 18.09 | O |
| ATOM | 3520 | N | ARG | C | 158 | 11.060 | −32.354 | −17.770 | 1.00 | 18.60 | N |
| ATOM | 3521 | CA | ARG | C | 158 | 11.725 | −31.541 | −16.759 | 1.00 | 19.14 | C |
| ATOM | 3522 | CB | ARG | C | 158 | 12.652 | −30.532 | −17.442 | 1.00 | 18.91 | C |
| ATOM | 3523 | CG | ARG | C | 158 | 13.543 | −29.723 | −16.522 | 1.00 | 18.43 | C |
| ATOM | 3524 | CD | ARG | C | 158 | 14.441 | −28.788 | −17.328 | 1.00 | 18.29 | C |
| ATOM | 3525 | NE | ARG | C | 158 | 15.283 | −29.527 | −18.276 | 1.00 | 18.50 | N |
| ATOM | 3526 | CZ | ARG | C | 158 | 16.441 | −30.116 | −17.967 | 1.00 | 18.82 | C |
| ATOM | 3527 | NH1 | ARG | C | 158 | 16.972 | −30.046 | −16.738 | 1.00 | 19.09 | N |
| ATOM | 3528 | NH2 | ARG | C | 158 | 17.110 | −30.766 | −18.912 | 1.00 | 18.58 | N |
| ATOM | 3529 | C | ARG | C | 158 | 10.719 | −30.794 | −15.902 | 1.00 | 19.66 | C |
| ATOM | 3530 | O | ARG | C | 158 | 9.731 | −30.299 | −16.414 | 1.00 | 19.25 | O |
| ATOM | 3531 | N | ALA | C | 159 | 10.998 | −30.697 | −14.604 | 1.00 | 21.14 | N |
| ATOM | 3532 | CA | ALA | C | 159 | 10.235 | −29.864 | −13.682 | 1.00 | 23.48 | C |
| ATOM | 3533 | CB | ALA | C | 159 | 9.593 | −30.716 | −12.592 | 1.00 | 23.76 | C |
| ATOM | 3534 | C | ALA | C | 159 | 11.163 | −28.810 | −13.073 | 1.00 | 25.02 | C |
| ATOM | 3535 | O | ALA | C | 159 | 12.317 | −29.110 | −12.792 | 1.00 | 24.80 | O |
| ATOM | 3536 | N | MET | C | 160 | 10.649 | −27.597 | −12.873 | 1.00 | 27.58 | N |
| ATOM | 3537 | CA | MET | C | 160 | 11.406 | −26.473 | −12.307 | 1.00 | 27.85 | C |
| ATOM | 3538 | CB | MET | C | 160 | 12.094 | −25.675 | −13.443 | 1.00 | 28.39 | C |
| ATOM | 3539 | CG | MET | C | 160 | 12.779 | −24.363 | −13.024 | 1.00 | 28.95 | C |
| ATOM | 3540 | SD | MET | C | 160 | 13.355 | −23.335 | −14.407 | 1.00 | 29.85 | S |
| ATOM | 3541 | CE | MET | C | 160 | 14.665 | −24.384 | −15.022 | 1.00 | 29.64 | C |
| ATOM | 3542 | C | MET | C | 160 | 10.455 | −25.561 | −11.532 | 1.00 | 29.05 | C |
| ATOM | 3543 | O | MET | C | 160 | 9.330 | −25.347 | −11.972 | 1.00 | 28.98 | O |
| ATOM | 3544 | N | ALA | C | 161 | 10.890 | −25.027 | −10.393 | 1.00 | 29.76 | N |
| ATOM | 3545 | CA | ALA | C | 161 | 10.086 | −24.072 | −9.614 | 1.00 | 31.67 | C |
| ATOM | 3546 | CB | ALA | C | 161 | 10.148 | −24.403 | −8.126 | 1.00 | 31.72 | C |
| ATOM | 3547 | C | ALA | C | 161 | 10.517 | −22.610 | −9.859 | 1.00 | 34.53 | C |
| ATOM | 3548 | O | ALA | C | 161 | 11.696 | −22.351 | −10.070 | 1.00 | 34.58 | O |
| ATOM | 3549 | N | ILE | C | 162 | 9.561 | −21.687 | −9.852 | 1.00 | 37.63 | N |
| ATOM | 3550 | CA | ILE | C | 162 | 9.830 | −20.244 | −9.833 | 1.00 | 41.55 | C |
| ATOM | 3551 | CB | ILE | C | 162 | 9.788 | −19.586 | −11.239 | 1.00 | 42.11 | C |
| ATOM | 3552 | CG1 | ILE | C | 162 | 8.399 | −19.695 | −11.882 | 1.00 | 42.77 | C |
| ATOM | 3553 | CD1 | ILE | C | 162 | 8.239 | −18.814 | −13.098 | 1.00 | 43.89 | C |
| ATOM | 3554 | CG2 | ILE | C | 162 | 10.853 | −20.183 | −12.136 | 1.00 | 42.49 | C |
| ATOM | 3555 | C | ILE | C | 162 | 8.818 | −19.529 | −8.930 | 1.00 | 44.02 | C |
| ATOM | 3556 | O | ILE | C | 162 | 7.703 | −20.016 | −8.726 | 1.00 | 41.79 | O |
| ATOM | 3557 | N | TYR | C | 163 | 9.216 | −18.359 | −8.429 | 1.00 | 49.03 | N |
| ATOM | 3558 | CA | TYR | C | 163 | 8.320 | −17.517 | −7.648 | 1.00 | 52.19 | C |
| ATOM | 3559 | CB | TYR | C | 163 | 9.109 | −16.512 | −6.786 | 1.00 | 51.83 | C |
| ATOM | 3560 | CG | TYR | C | 163 | 9.940 | −17.196 | −5.729 | 1.00 | 51.45 | C |
| ATOM | 3561 | CD1 | TYR | C | 163 | 9.335 | −17.970 | −4.716 | 1.00 | 52.04 | C |
| ATOM | 3562 | CE1 | TYR | C | 163 | 10.098 | −18.600 | −3.752 | 1.00 | 52.25 | C |
| ATOM | 3563 | CZ | TYR | C | 163 | 11.482 | −18.473 | −3.778 | 1.00 | 52.53 | C |
| ATOM | 3564 | OH | TYR | C | 163 | 12.242 | −19.111 | −2.827 | 1.00 | 53.56 | O |
| ATOM | 3565 | CE2 | TYR | C | 163 | 12.097 | −17.716 | −4.761 | 1.00 | 51.88 | C |
| ATOM | 3566 | CD2 | TYR | C | 163 | 11.322 | −17.088 | −5.732 | 1.00 | 51.19 | C |
| ATOM | 3567 | C | TYR | C | 163 | 7.350 | −16.782 | −8.568 | 1.00 | 56.31 | C |
| ATOM | 3568 | O | TYR | C | 163 | 7.752 | −16.330 | −9.644 | 1.00 | 53.83 | O |
| ATOM | 3569 | N | LYS | C | 164 | 6.100 | −16.651 | −8.120 | 1.00 | 63.18 | N |
| ATOM | 3570 | CA | LYS | C | 164 | 5.059 | −15.975 | −8.893 | 1.00 | 69.50 | C |
| ATOM | 3571 | CB | LYS | C | 164 | 3.660 | −16.362 | −8.369 | 1.00 | 71.32 | C |
| ATOM | 3572 | CG | LYS | C | 164 | 2.502 | −15.939 | −9.280 | 1.00 | 73.00 | C |
| ATOM | 3573 | CD | LYS | C | 164 | 1.152 | −16.033 | −8.591 | 1.00 | 74.33 | C |
| ATOM | 3574 | CE | LYS | C | 164 | 0.075 | −15.423 | −9.475 | 1.00 | 75.29 | C |
| ATOM | 3575 | NZ | LYS | C | 164 | −1.271 | −15.416 | −8.839 | 1.00 | 75.34 | N |
| ATOM | 3576 | C | LYS | C | 164 | 5.189 | −14.444 | −8.939 | 1.00 | 72.24 | C |
| ATOM | 3577 | O | LYS | C | 164 | 4.958 | −13.847 | −9.992 | 1.00 | 73.72 | O |
| ATOM | 3578 | N | GLN | C | 165 | 5.541 | −13.831 | −7.816 | 1.00 | 76.54 | N |
| ATOM | 3579 | CA | GLN | C | 165 | 5.457 | −12.348 | −7.712 | 1.00 | 77.60 | C |
| ATOM | 3580 | CB | GLN | C | 165 | 5.453 | −11.860 | −6.246 | 1.00 | 79.65 | C |
| ATOM | 3581 | CG | GLN | C | 165 | 4.336 | −12.479 | −5.397 | 1.00 | 80.70 | C |
| ATOM | 3582 | CD | GLN | C | 165 | 3.968 | −11.664 | −4.174 | 1.00 | 80.19 | C |
| ATOM | 3583 | OE1 | GLN | C | 165 | 3.914 | −10.458 | −4.262 | 1.00 | 78.91 | O |
| ATOM | 3584 | NE2 | GLN | C | 165 | 3.640 | −12.340 | −3.054 | 1.00 | 77.96 | N |
| ATOM | 3585 | C | GLN | C | 165 | 6.580 | −11.680 | −8.486 | 1.00 | 77.49 | C |
| ATOM | 3586 | O | GLN | C | 165 | 7.720 | −12.063 | −8.343 | 1.00 | 76.43 | O |
| ATOM | 3587 | N | SER | C | 166 | 6.244 | −10.680 | −9.297 | 1.00 | 78.04 | N |
| ATOM | 3588 | CA | SER | C | 166 | 7.188 | −10.042 | −10.241 | 1.00 | 77.04 | C |
| ATOM | 3589 | CB | SER | C | 166 | 6.495 | −8.910 | −11.013 | 1.00 | 76.61 | C |
| ATOM | 3590 | OG | SER | C | 166 | 5.815 | −8.027 | −10.139 | 1.00 | 76.09 | O |
| ATOM | 3591 | C | SER | C | 166 | 8.486 | −9.532 | −9.605 | 1.00 | 78.76 | C |
| ATOM | 3592 | O | SER | C | 166 | 9.543 | −9.612 | −10.220 | 1.00 | 79.59 | O |
| ATOM | 3593 | N | GLN | C | 167 | 8.402 | −9.022 | −8.380 | 1.00 | 80.88 | N |
| ATOM | 3594 | CA | GLN | C | 167 | 9.595 | −8.537 | −7.654 | 1.00 | 83.41 | C |
| ATOM | 3595 | CB | GLN | C | 167 | 9.203 | −7.591 | −6.500 | 1.00 | 84.77 | C |
| ATOM | 3596 | CG | GLN | C | 167 | 8.568 | −8.232 | −5.262 | 1.00 | 84.83 | C |
| ATOM | 3597 | CD | GLN | C | 167 | 7.063 | −8.459 | −5.377 | 1.00 | 86.44 | C |
| ATOM | 3598 | OE1 | GLN | C | 167 | 6.489 | −8.433 | −6.466 | 1.00 | 84.92 | O |
| ATOM | 3599 | NE2 | GLN | C | 167 | 6.414 | −8.688 | −4.242 | 1.00 | 87.39 | N |
| ATOM | 3600 | C | GLN | C | 167 | 10.542 | −9.649 | −7.148 | 1.00 | 84.24 | C |
| ATOM | 3601 | O | GLN | C | 167 | 11.697 | −9.370 | −6.842 | 1.00 | 82.52 | O |
| ATOM | 3602 | N | HIS | C | 168 | 10.038 | −10.885 | −7.047 | 1.00 | 84.70 | N |
| ATOM | 3603 | CA | HIS | C | 168 | 10.836 | −12.051 | −6.633 | 1.00 | 84.42 | C |
| ATOM | 3604 | CB | HIS | C | 168 | 10.141 | −12.805 | −5.495 | 1.00 | 84.88 | C |
| ATOM | 3605 | CG | HIS | C | 168 | 10.030 | −12.008 | −4.232 | 1.00 | 86.97 | C |
| ATOM | 3606 | ND1 | HIS | C | 168 | 11.125 | −11.471 | −3.590 | 1.00 | 87.16 | N |
| ATOM | 3607 | CE1 | HIS | C | 168 | 10.725 | −10.807 | −2.522 | 1.00 | 87.63 | C |
| ATOM | 3608 | NE2 | HIS | C | 168 | 9.408 | −10.883 | −2.459 | 1.00 | 86.91 | N |
| ATOM | 3609 | CD2 | HIS | C | 168 | 8.951 | −11.618 | −3.527 | 1.00 | 87.58 | C |
| ATOM | 3610 | C | HIS | C | 168 | 11.142 | −13.056 | −7.750 | 1.00 | 82.78 | C |
| ATOM | 3611 | O | HIS | C | 168 | 11.919 | −13.990 | −7.528 | 1.00 | 83.42 | O |
| ATOM | 3612 | N | MET | C | 169 | 10.599 | −12.850 | −8.959 | 1.00 | 81.46 | N |
| ATOM | 3613 | CA | MET | C | 169 | 10.739 | −13.819 | −10.064 | 1.00 | 81.99 | C |
| ATOM | 3614 | CB | MET | C | 169 | 9.948 | −13.349 | −11.294 | 1.00 | 83.61 | C |
| ATOM | 3615 | CG | MET | C | 169 | 9.856 | −14.339 | −12.451 | 1.00 | 86.05 | C |
| ATOM | 3616 | SD | MET | C | 169 | 8.369 | −14.234 | −13.466 | 1.00 | 89.82 | S |
| ATOM | 3617 | CE | MET | C | 169 | 7.199 | −15.070 | −12.392 | 1.00 | 89.44 | C |
| ATOM | 3618 | C | MET | C | 169 | 12.201 | −14.110 | −10.471 | 1.00 | 80.24 | C |
| ATOM | 3619 | O | MET | C | 169 | 12.477 | −15.200 | −10.969 | 1.00 | 81.04 | O |
| ATOM | 3620 | N | THR | C | 170 | 13.117 | −13.160 | −10.264 | 1.00 | 75.78 | N |
| ATOM | 3621 | CA | THR | C | 170 | 14.529 | −13.365 | −10.598 | 1.00 | 72.72 | C |
| ATOM | 3622 | CB | THR | C | 170 | 15.260 | −12.030 | −10.859 | 1.00 | 71.23 | C |
| ATOM | 3623 | OG1 | THR | C | 170 | 15.239 | −11.229 | −9.673 | 1.00 | 70.97 | O |
| ATOM | 3624 | CG2 | THR | C | 170 | 14.598 | −11.274 | −11.994 | 1.00 | 70.69 | C |
| ATOM | 3625 | C | THR | C | 170 | 15.312 | −14.163 | −9.552 | 1.00 | 70.37 | C |
| ATOM | 3626 | O | THR | C | 170 | 16.392 | −14.666 | −9.844 | 1.00 | 70.79 | O |
| ATOM | 3627 | N | GLU | C | 171 | 14.781 | −14.269 | −8.341 | 1.00 | 67.86 | N |
| ATOM | 3628 | CA | GLU | C | 171 | 15.416 | −15.067 | −7.275 | 1.00 | 67.18 | C |
| ATOM | 3629 | CB | GLU | C | 171 | 14.852 | −14.720 | −5.900 | 1.00 | 69.51 | C |
| ATOM | 3630 | CG | GLU | C | 171 | 15.025 | −13.248 | −5.506 | 1.00 | 71.36 | C |
| ATOM | 3631 | CD | GLU | C | 171 | 14.888 | −12.974 | −4.027 | 1.00 | 72.06 | C |
| ATOM | 3632 | OE1 | GLU | C | 171 | 15.716 | −12.198 | −3.507 | 1.00 | 72.10 | O |
| ATOM | 3633 | OE2 | GLU | C | 171 | 13.952 | −13.502 | −3.384 | 1.00 | 72.21 | O |
| ATOM | 3634 | C | GLU | C | 171 | 15.212 | −16.556 | −7.534 | 1.00 | 65.35 | C |
| ATOM | 3635 | O | GLU | C | 171 | 14.152 | −16.994 | −7.998 | 1.00 | 67.29 | O |
| ATOM | 3636 | N | VAL | C | 172 | 16.249 | −17.336 | −7.246 | 1.00 | 62.76 | N |
| ATOM | 3637 | CA | VAL | C | 172 | 16.227 | −18.776 | −7.507 | 1.00 | 61.07 | C |
| ATOM | 3638 | CB | VAL | C | 172 | 17.648 | −19.373 | −7.599 | 1.00 | 61.99 | C |
| ATOM | 3639 | CG1 | VAL | C | 172 | 17.586 | −20.875 | −7.818 | 1.00 | 61.26 | C |
| ATOM | 3640 | CG2 | VAL | C | 172 | 18.429 | −18.725 | −8.739 | 1.00 | 61.17 | C |
| ATOM | 3641 | C | VAL | C | 172 | 15.448 | −19.461 | −6.402 | 1.00 | 58.84 | C |
| ATOM | 3642 | O | VAL | C | 172 | 15.760 | −19.271 | −5.231 | 1.00 | 59.16 | O |
| ATOM | 3643 | N | VAL | C | 173 | 14.447 | −20.251 | −6.770 | 1.00 | 58.93 | N |
| ATOM | 3644 | CA | VAL | C | 173 | 13.720 | −21.069 | −5.792 | 1.00 | 58.83 | C |
| ATOM | 3645 | CB | VAL | C | 173 | 12.413 | −21.666 | −6.351 | 1.00 | 57.50 | C |
| ATOM | 3646 | CG1 | VAL | C | 173 | 11.767 | −22.601 | −5.337 | 1.00 | 58.56 | C |
| ATOM | 3647 | CG2 | VAL | C | 173 | 11.442 | −20.578 | −6.746 | 1.00 | 58.22 | C |
| ATOM | 3648 | C | VAL | C | 173 | 14.649 | −22.215 | −5.385 | 1.00 | 58.41 | C |
| ATOM | 3649 | O | VAL | C | 173 | 15.003 | −23.068 | −6.202 | 1.00 | 60.45 | O |
| ATOM | 3650 | N | ARG | C | 174 | 15.044 | −22.205 | −4.107 | 1.00 | 58.56 | N |
| ATOM | 3651 | CA | ARG | C | 174 | 15.766 | −23.318 | −3.503 | 1.00 | 60.13 | C |
| ATOM | 3652 | CB | ARG | C | 174 | 17.165 | −22.865 | −3.113 | 1.00 | 60.16 | C |
| ATOM | 3653 | CG | ARG | C | 174 | 18.000 | −22.119 | −4.168 | 1.00 | 61.72 | C |
| ATOM | 3654 | CD | ARG | C | 174 | 19.453 | −21.942 | −3.756 | 1.00 | 62.30 | C |
| ATOM | 3655 | NE | ARG | C | 174 | 20.351 | −21.681 | −4.893 | 1.00 | 60.78 | N |
| ATOM | 3656 | CZ | ARG | C | 174 | 20.755 | −20.480 | −5.332 | 1.00 | 61.10 | C |
| ATOM | 3657 | NH1 | ARG | C | 174 | 20.350 | −19.340 | −4.766 | 1.00 | 62.14 | N |
| ATOM | 3658 | NH2 | ARG | C | 174 | 21.568 | −20.418 | −6.386 | 1.00 | 60.60 | N |
| ATOM | 3659 | C | ARG | C | 174 | 14.995 | −23.806 | −2.268 | 1.00 | 61.32 | C |
| ATOM | 3660 | O | ARG | C | 174 | 14.082 | −23.142 | −1.777 | 1.00 | 62.94 | O |
| ATOM | 3661 | N | ARG | C | 175 | 15.370 | −24.978 | −1.797 | 1.00 | 62.84 | N |
| ATOM | 3662 | CA | ARG | C | 175 | 14.803 | −25.562 | −0.566 | 1.00 | 63.15 | C |
| ATOM | 3663 | CB | ARG | C | 175 | 15.302 | −26.991 | −0.404 | 1.00 | 59.69 | C |
| ATOM | 3664 | CG | ARG | C | 175 | 14.570 | −27.856 | 0.565 | 1.00 | 58.71 | C |
| ATOM | 3665 | CD | ARG | C | 175 | 15.215 | −29.233 | 0.514 | 1.00 | 57.13 | C |
| ATOM | 3666 | NE | ARG | C | 175 | 14.806 | −30.133 | 1.593 | 1.00 | 54.97 | N |
| ATOM | 3667 | CZ | ARG | C | 175 | 15.499 | −31.183 | 2.048 | 1.00 | 55.21 | C |
| ATOM | 3668 | NH1 | ARG | C | 175 | 14.978 | −31.934 | 3.017 | 1.00 | 54.99 | N |
| ATOM | 3669 | NH2 | ARG | C | 175 | 16.689 | −31.503 | 1.540 | 1.00 | 55.64 | N |
| ATOM | 3670 | C | ARG | C | 175 | 15.236 | −24.729 | 0.643 | 1.00 | 67.37 | C |
| ATOM | 3671 | O | ARG | C | 175 | 16.320 | −24.095 | 0.619 | 1.00 | 69.33 | O |
| ATOM | 3672 | N | CYS | C | 176 | 14.407 | −24.718 | 1.687 | 1.00 | 71.76 | N |
| ATOM | 3673 | CA | CYS | C | 176 | 14.758 | −23.995 | 2.924 | 1.00 | 75.39 | C |
| ATOM | 3674 | CB | CYS | C | 176 | 13.601 | −23.997 | 3.924 | 1.00 | 74.76 | C |
| ATOM | 3675 | SG | CYS | C | 176 | 13.142 | −25.628 | 4.575 | 1.00 | 72.99 | S |
| ATOM | 3676 | C | CYS | C | 176 | 16.046 | −24.571 | 3.566 | 1.00 | 78.73 | C |
| ATOM | 3677 | O | CYS | C | 176 | 16.235 | −25.803 | 3.601 | 1.00 | 79.51 | O |
| ATOM | 3678 | N | PRO | C | 177 | 17.023 | −23.683 | 3.908 | 1.00 | 80.98 | N |
| ATOM | 3679 | CA | PRO | C | 177 | 18.325 | −24.230 | 4.328 | 1.00 | 81.22 | C |
| ATOM | 3680 | CB | PRO | C | 177 | 19.176 | −22.979 | 4.508 | 1.00 | 81.51 | C |
| ATOM | 3681 | CG | PRO | C | 177 | 18.651 | −22.039 | 3.485 | 1.00 | 81.80 | C |
| ATOM | 3682 | CD | PRO | C | 177 | 17.154 | −22.281 | 3.464 | 1.00 | 80.87 | C |
| ATOM | 3683 | C | PRO | C | 177 | 18.212 | −25.057 | 5.613 | 1.00 | 80.35 | C |
| ATOM | 3684 | O | PRO | C | 177 | 18.872 | −26.091 | 5.749 | 1.00 | 79.66 | O |
| ATOM | 3685 | N | ALA | C | 189 | 19.462 | −37.146 | −4.888 | 1.00 | 47.18 | N |
| ATOM | 3686 | CA | ALA | C | 189 | 18.968 | −35.775 | −5.070 | 1.00 | 48.30 | C |
| ATOM | 3687 | CB | ALA | C | 189 | 17.527 | −35.675 | −4.608 | 1.00 | 49.20 | C |
| ATOM | 3688 | C | ALA | C | 189 | 19.828 | −34.738 | −4.309 | 1.00 | 48.59 | C |
| ATOM | 3689 | O | ALA | C | 189 | 20.240 | −34.998 | −3.175 | 1.00 | 48.98 | O |
| ATOM | 3690 | N | PRO | C | 190 | 20.116 | −33.593 | −4.948 | 1.00 | 48.17 | N |
| ATOM | 3691 | CA | PRO | C | 190 | 20.884 | −32.544 | −4.267 | 1.00 | 48.70 | C |
| ATOM | 3692 | CB | PRO | C | 190 | 21.064 | −31.461 | −5.339 | 1.00 | 47.87 | C |
| ATOM | 3693 | CG | PRO | C | 190 | 20.806 | −32.123 | −6.639 | 1.00 | 48.87 | C |
| ATOM | 3694 | CD | PRO | C | 190 | 19.904 | −33.295 | −6.376 | 1.00 | 47.82 | C |
| ATOM | 3695 | C | PRO | C | 190 | 20.129 | −31.958 | −3.075 | 1.00 | 49.28 | C |
| ATOM | 3696 | O | PRO | C | 190 | 18.907 | −31.841 | −3.145 | 1.00 | 49.76 | O |
| ATOM | 3697 | N | PRO | C | 191 | 20.843 | −31.588 | −1.995 | 1.00 | 50.45 | N |
| ATOM | 3698 | CA | PRO | C | 191 | 20.160 | −31.084 | −0.796 | 1.00 | 50.86 | C |
| ATOM | 3699 | CB | PRO | C | 191 | 21.275 | −31.008 | 0.261 | 1.00 | 51.98 | C |
| ATOM | 3700 | CG | PRO | C | 191 | 22.567 | −31.005 | −0.504 | 1.00 | 52.23 | C |
| ATOM | 3701 | CD | PRO | C | 191 | 22.305 | −31.710 | −1.804 | 1.00 | 52.19 | C |
| ATOM | 3702 | C | PRO | C | 191 | 19.465 | −29.721 | −0.952 | 1.00 | 49.87 | C |
| ATOM | 3703 | O | PRO | C | 191 | 18.529 | −29.443 | −0.181 | 1.00 | 49.58 | O |
| ATOM | 3704 | N | GLN | C | 192 | 19.885 | −28.904 | −1.925 | 1.00 | 47.46 | N |
| ATOM | 3705 | CA | GLN | C | 192 | 19.256 | −27.600 | −2.168 | 1.00 | 47.73 | C |
| ATOM | 3706 | CB | GLN | C | 192 | 20.240 | −26.637 | −2.850 | 1.00 | 50.65 | C |
| ATOM | 3707 | CG | GLN | C | 192 | 21.416 | −26.201 | −1.989 | 1.00 | 52.42 | C |
| ATOM | 3708 | CD | GLN | C | 192 | 22.028 | −24.881 | −2.487 | 1.00 | 55.43 | C |
| ATOM | 3709 | OE1 | GLN | C | 192 | 21.995 | −24.581 | −3.699 | 1.00 | 54.63 | O |
| ATOM | 3710 | NE2 | GLN | C | 192 | 22.583 | −24.083 | −1.555 | 1.00 | 56.30 | N |
| ATOM | 3711 | C | GLN | C | 192 | 17.970 | −27.630 | −3.007 | 1.00 | 45.99 | C |
| ATOM | 3712 | O | GLN | C | 192 | 17.234 | −26.649 | −2.995 | 1.00 | 46.97 | O |
| ATOM | 3713 | N | HIS | C | 193 | 17.694 | −28.723 | −3.727 | 1.00 | 43.56 | N |
| ATOM | 3714 | CA | HIS | C | 193 | 16.564 | −28.763 | −4.683 | 1.00 | 40.87 | C |
| ATOM | 3715 | CB | HIS | C | 193 | 16.738 | −29.876 | −5.721 | 1.00 | 38.93 | C |
| ATOM | 3716 | CG | HIS | C | 193 | 17.759 | −29.568 | −6.773 | 1.00 | 37.37 | C |
| ATOM | 3717 | ND1 | HIS | C | 193 | 17.580 | −29.909 | −8.095 | 1.00 | 36.52 | N |
| ATOM | 3718 | CE1 | HIS | C | 193 | 18.642 | −29.541 | −8.787 | 1.00 | 36.98 | C |
| ATOM | 3719 | NE2 | HIS | C | 193 | 19.504 | −28.961 | −7.965 | 1.00 | 35.81 | N |
| ATOM | 3720 | CD2 | HIS | C | 193 | 18.977 | −28.966 | −6.701 | 1.00 | 35.99 | C |
| ATOM | 3721 | C | HIS | C | 193 | 15.227 | −28.965 | −3.980 | 1.00 | 39.53 | C |
| ATOM | 3722 | O | HIS | C | 193 | 15.076 | −29.871 | −3.154 | 1.00 | 39.02 | O |
| ATOM | 3723 | N | LEU | C | 194 | 14.266 | −28.108 | −4.319 | 1.00 | 38.47 | N |
| ATOM | 3724 | CA | LEU | C | 194 | 12.899 | −28.218 | −3.806 | 1.00 | 36.74 | C |
| ATOM | 3725 | CB | LEU | C | 194 | 12.090 | −26.964 | −4.154 | 1.00 | 36.66 | C |
| ATOM | 3726 | CG | LEU | C | 194 | 10.626 | −26.905 | −3.723 | 1.00 | 36.30 | C |
| ATOM | 3727 | CD1 | LEU | C | 194 | 10.469 | −27.048 | −2.206 | 1.00 | 36.50 | C |
| ATOM | 3728 | CD2 | LEU | C | 194 | 10.043 | −25.542 | −4.165 | 1.00 | 35.73 | C |
| ATOM | 3729 | C | LEU | C | 194 | 12.181 | −29.448 | −4.352 | 1.00 | 36.34 | C |
| ATOM | 3730 | O | LEU | C | 194 | 11.561 | −30.187 | −3.606 | 1.00 | 35.12 | O |
| ATOM | 3731 | N | ILE | C | 195 | 12.264 | −29.662 | −5.663 | 1.00 | 36.23 | N |
| ATOM | 3732 | CA | ILE | C | 195 | 11.515 | −30.716 | −6.346 | 1.00 | 34.86 | C |
| ATOM | 3733 | CB | ILE | C | 195 | 10.923 | −30.220 | −7.688 | 1.00 | 35.40 | C |
| ATOM | 3734 | CG1 | ILE | C | 195 | 10.198 | −28.870 | −7.506 | 1.00 | 34.69 | C |
| ATOM | 3735 | CD1 | ILE | C | 195 | 9.821 | −28.168 | −8.786 | 1.00 | 35.09 | C |
| ATOM | 3736 | CG2 | ILE | C | 195 | 9.984 | −31.277 | −8.280 | 1.00 | 35.40 | C |
| ATOM | 3737 | C | ILE | C | 195 | 12.395 | −31.933 | −6.603 | 1.00 | 34.59 | C |
| ATOM | 3738 | O | ILE | C | 195 | 13.467 | −31.821 | −7.202 | 1.00 | 33.45 | O |
| ATOM | 3739 | N | ARG | C | 196 | 11.930 | −33.091 | −6.139 | 1.00 | 35.91 | N |
| ATOM | 3740 | CA | ARG | C | 196 | 12.538 | −34.390 | −6.447 | 1.00 | 36.87 | C |
| ATOM | 3741 | CB | ARG | C | 196 | 12.904 | −35.131 | −5.165 | 1.00 | 39.36 | C |
| ATOM | 3742 | CG | ARG | C | 196 | 13.915 | −34.410 | −4.304 | 1.00 | 41.45 | C |
| ATOM | 3743 | CD | ARG | C | 196 | 14.262 | −35.185 | −3.051 | 1.00 | 43.69 | C |
| ATOM | 3744 | NE | ARG | C | 196 | 15.202 | −34.422 | −2.229 | 1.00 | 47.03 | N |
| ATOM | 3745 | CZ | ARG | C | 196 | 15.726 | −34.820 | −1.063 | 1.00 | 49.27 | C |
| ATOM | 3746 | NH1 | ARG | C | 196 | 15.430 | −36.011 | −0.556 | 1.00 | 48.39 | N |
| ATOM | 3747 | NH2 | ARG | C | 196 | 16.606 | −34.035 | −0.452 | 1.00 | 49.95 | N |
| ATOM | 3748 | C | ARG | C | 196 | 11.541 | −35.237 | −7.205 | 1.00 | 36.07 | C |
| ATOM | 3749 | O | ARG | C | 196 | 10.341 | −34.947 | −7.213 | 1.00 | 35.07 | O |
| ATOM | 3750 | N | VAL | C | 197 | 12.039 | −36.297 | −7.832 | 1.00 | 35.58 | N |
| ATOM | 3751 | CA | VAL | C | 197 | 11.192 | −37.349 | −8.385 | 1.00 | 35.28 | C |
| ATOM | 3752 | CB | VAL | C | 197 | 11.505 | −37.604 | −9.875 | 1.00 | 33.72 | C |
| ATOM | 3753 | CG1 | VAL | C | 197 | 10.766 | −38.841 | −10.392 | 1.00 | 33.24 | C |
| ATOM | 3754 | CG2 | VAL | C | 197 | 11.136 | −36.375 | −10.692 | 1.00 | 33.60 | C |
| ATOM | 3755 | C | VAL | C | 197 | 11.424 | −38.617 | −7.575 | 1.00 | 36.02 | C |
| ATOM | 3756 | O | VAL | C | 197 | 12.567 | −38.953 | −7.242 | 1.00 | 35.48 | O |
| ATOM | 3757 | N | GLU | C | 198 | 10.329 | −39.309 | −7.280 | 1.00 | 38.38 | N |
| ATOM | 3758 | CA | GLU | C | 198 | 10.328 | −40.522 | −6.479 | 1.00 | 41.15 | C |
| ATOM | 3759 | CB | GLU | C | 198 | 9.142 | −40.466 | −5.520 | 1.00 | 43.50 | C |
| ATOM | 3760 | CG | GLU | C | 198 | 9.119 | −41.538 | −4.436 | 1.00 | 44.82 | C |
| ATOM | 3761 | CD | GLU | C | 198 | 7.841 | −41.528 | −3.617 | 1.00 | 45.70 | C |
| ATOM | 3762 | OE1 | GLU | C | 198 | 7.073 | −40.544 | −3.693 | 1.00 | 44.87 | O |
| ATOM | 3763 | OE2 | GLU | C | 198 | 7.615 | −42.480 | −2.855 | 1.00 | 48.23 | O |
| ATOM | 3764 | C | GLU | C | 198 | 10.194 | −41.734 | −7.397 | 1.00 | 41.67 | C |
| ATOM | 3765 | O | GLU | C | 198 | 9.566 | −41.642 | −8.453 | 1.00 | 43.58 | O |
| ATOM | 3766 | N | GLY | C | 199 | 10.780 | −42.859 | −6.990 | 1.00 | 43.26 | N |
| ATOM | 3767 | CA | GLY | C | 199 | 10.593 | −44.144 | −7.682 | 1.00 | 43.49 | C |
| ATOM | 3768 | C | GLY | C | 199 | 11.011 | −44.178 | −9.144 | 1.00 | 43.51 | C |
| ATOM | 3769 | O | GLY | C | 199 | 10.359 | −44.825 | −9.959 | 1.00 | 43.59 | O |
| ATOM | 3770 | N | ASN | C | 200 | 12.093 | −43.473 | −9.477 | 1.00 | 42.97 | N |
| ATOM | 3771 | CA | ASN | C | 200 | 12.641 | −43.473 | −10.837 | 1.00 | 41.51 | C |
| ATOM | 3772 | CB | ASN | C | 200 | 12.046 | −42.326 | −11.679 | 1.00 | 40.81 | C |
| ATOM | 3773 | CG | ASN | C | 200 | 12.306 | −42.496 | −13.172 | 1.00 | 38.27 | C |
| ATOM | 3774 | OD1 | ASN | C | 200 | 13.390 | −42.904 | −13.580 | 1.00 | 38.24 | O |
| ATOM | 3775 | ND2 | ASN | C | 200 | 11.314 | −42.159 | −13.992 | 1.00 | 36.52 | N |
| ATOM | 3776 | C | ASN | C | 200 | 14.168 | −43.398 | −10.785 | 1.00 | 40.90 | C |
| ATOM | 3777 | O | ASN | C | 200 | 14.743 | −42.338 | −10.533 | 1.00 | 39.14 | O |
| ATOM | 3778 | N | LEU | C | 201 | 14.800 | −44.537 | −11.055 | 1.00 | 43.06 | N |
| ATOM | 3779 | CA | LEU | C | 201 | 16.252 | −44.698 | −10.959 | 1.00 | 44.50 | C |
| ATOM | 3780 | CB | LEU | C | 201 | 16.606 | −46.199 | −11.020 | 1.00 | 47.94 | C |
| ATOM | 3781 | CG | LEU | C | 201 | 17.951 | −46.634 | −10.439 | 1.00 | 51.60 | C |
| ATOM | 3782 | CD1 | LEU | C | 201 | 17.733 | −46.761 | −8.941 | 1.00 | 52.37 | C |
| ATOM | 3783 | CD2 | LEU | C | 201 | 18.440 | −47.945 | −11.051 | 1.00 | 52.09 | C |
| ATOM | 3784 | C | LEU | C | 201 | 17.033 | −43.949 | −12.058 | 1.00 | 42.89 | C |
| ATOM | 3785 | O | LEU | C | 201 | 18.232 | −43.742 | −11.913 | 1.00 | 42.20 | O |
| ATOM | 3786 | N | ARG | C | 202 | 16.355 | −43.562 | −13.150 | 1.00 | 41.23 | N |
| ATOM | 3787 | CA | ARG | C | 202 | 16.963 | −42.775 | −14.226 | 1.00 | 40.15 | C |
| ATOM | 3788 | CB | ARG | C | 202 | 16.345 | −43.189 | −15.572 | 1.00 | 42.14 | C |
| ATOM | 3789 | CG | ARG | C | 202 | 16.649 | −44.635 | −15.959 | 1.00 | 44.74 | C |
| ATOM | 3790 | CD | ARG | C | 202 | 15.527 | −45.277 | −16.770 | 1.00 | 45.46 | C |
| ATOM | 3791 | NE | ARG | C | 202 | 15.661 | −45.066 | −18.211 | 1.00 | 47.85 | N |
| ATOM | 3792 | CZ | ARG | C | 202 | 14.671 | −45.192 | −19.105 | 1.00 | 48.94 | C |
| ATOM | 3793 | NH1 | ARG | C | 202 | 13.417 | −45.478 | −18.730 | 1.00 | 49.38 | N |
| ATOM | 3794 | NH2 | ARG | C | 202 | 14.931 | −44.999 | −20.396 | 1.00 | 48.34 | N |
| ATOM | 3795 | C | ARG | C | 202 | 16.840 | −41.252 | −14.033 | 1.00 | 37.43 | C |
| ATOM | 3796 | O | ARG | C | 202 | 17.055 | −40.482 | −14.978 | 1.00 | 37.54 | O |
| ATOM | 3797 | N | VAL | C | 203 | 16.476 | −40.810 | −12.829 | 1.00 | 34.32 | N |
| ATOM | 3798 | CA | VAL | C | 203 | 16.338 | −39.378 | −12.512 | 1.00 | 33.11 | C |
| ATOM | 3799 | CB | VAL | C | 203 | 15.725 | −39.173 | −11.097 | 1.00 | 33.35 | C |
| ATOM | 3800 | CG1 | VAL | C | 203 | 16.707 | −39.565 | −9.995 | 1.00 | 33.44 | C |
| ATOM | 3801 | CG2 | VAL | C | 203 | 15.241 | −37.736 | −10.906 | 1.00 | 32.97 | C |
| ATOM | 3802 | C | VAL | C | 203 | 17.677 | −38.628 | −12.635 | 1.00 | 30.83 | C |
| ATOM | 3803 | O | VAL | C | 203 | 18.731 | −39.168 | −12.311 | 1.00 | 29.76 | O |
| ATOM | 3804 | N | GLU | C | 204 | 17.609 | −37.387 | −13.112 | 1.00 | 28.75 | N |
| ATOM | 3805 | CA | GLU | C | 204 | 18.784 | −36.539 | −13.310 | 1.00 | 27.44 | C |
| ATOM | 3806 | CB | GLU | C | 204 | 19.154 | −36.510 | −14.803 | 1.00 | 27.19 | C |
| ATOM | 3807 | CG | GLU | C | 204 | 20.119 | −35.408 | −15.245 | 1.00 | 27.42 | C |
| ATOM | 3808 | CD | GLU | C | 204 | 20.303 | −35.363 | −16.753 | 1.00 | 26.77 | C |
| ATOM | 3809 | OE1 | GLU | C | 204 | 20.368 | −36.441 | −17.372 | 1.00 | 25.73 | O |
| ATOM | 3810 | OE2 | GLU | C | 204 | 20.380 | −34.252 | −17.330 | 1.00 | 27.34 | O |
| ATOM | 3811 | C | GLU | C | 204 | 18.487 | −35.144 | −12.799 | 1.00 | 25.81 | C |
| ATOM | 3812 | O | GLU | C | 204 | 17.425 | −34.604 | −13.089 | 1.00 | 25.71 | O |
| ATOM | 3813 | N | TYR | C | 205 | 19.438 | −34.564 | −12.069 | 1.00 | 25.71 | N |
| ATOM | 3814 | CA | TYR | C | 205 | 19.316 | −33.205 | −11.528 | 1.00 | 25.43 | C |
| ATOM | 3815 | CB | TYR | C | 205 | 19.508 | −33.232 | −10.008 | 1.00 | 25.32 | C |
| ATOM | 3816 | CG | TYR | C | 205 | 18.402 | −33.981 | −9.301 | 1.00 | 24.90 | C |
| ATOM | 3817 | CD1 | TYR | C | 205 | 18.478 | −35.360 | −9.122 | 1.00 | 24.58 | C |
| ATOM | 3818 | CE1 | TYR | C | 205 | 17.457 | −36.063 | −8.512 | 1.00 | 24.54 | C |
| ATOM | 3819 | CZ | TYR | C | 205 | 16.355 | −35.379 | −8.023 | 1.00 | 24.06 | C |
| ATOM | 3820 | OH | TYR | C | 205 | 15.330 | −36.057 | −7.395 | 1.00 | 25.77 | O |
| ATOM | 3821 | CE2 | TYR | C | 205 | 16.259 | −34.005 | −8.171 | 1.00 | 24.17 | C |
| ATOM | 3822 | CD2 | TYR | C | 205 | 17.274 | −33.315 | −8.827 | 1.00 | 24.11 | C |
| ATOM | 3823 | C | TYR | C | 205 | 20.310 | −32.244 | −12.169 | 1.00 | 25.53 | C |
| ATOM | 3824 | O | TYR | C | 205 | 21.422 | −32.636 | −12.511 | 1.00 | 24.72 | O |
| ATOM | 3825 | N | LEU | C | 206 | 19.904 | −30.983 | −12.304 | 1.00 | 26.97 | N |
| ATOM | 3826 | CA | LEU | C | 206 | 20.697 | −29.933 | −12.947 | 1.00 | 28.49 | C |
| ATOM | 3827 | CB | LEU | C | 206 | 20.133 | −29.611 | −14.343 | 1.00 | 27.07 | C |
| ATOM | 3828 | CG | LEU | C | 206 | 20.814 | −28.546 | −15.221 | 1.00 | 26.04 | C |
| ATOM | 3829 | CD1 | LEU | C | 206 | 22.186 | −28.997 | −15.690 | 1.00 | 25.66 | C |
| ATOM | 3830 | CD2 | LEU | C | 206 | 19.956 | −28.195 | −16.426 | 1.00 | 25.46 | C |
| ATOM | 3831 | C | LEU | C | 206 | 20.701 | −28.662 | −12.102 | 1.00 | 31.48 | C |
| ATOM | 3832 | O | LEU | C | 206 | 19.645 | −28.192 | −11.659 | 1.00 | 31.07 | O |
| ATOM | 3833 | N | ASP | C | 207 | 21.896 | −28.138 | −11.850 | 1.00 | 35.99 | N |
| ATOM | 3834 | CA | ASP | C | 207 | 22.081 | −26.743 | −11.492 | 1.00 | 39.30 | C |
| ATOM | 3835 | CB | ASP | C | 207 | 23.180 | −26.571 | −10.444 | 1.00 | 39.98 | C |
| ATOM | 3836 | CG | ASP | C | 207 | 22.817 | −27.173 | −9.091 | 1.00 | 41.01 | C |
| ATOM | 3837 | OD1 | ASP | C | 207 | 21.641 | −27.526 | −8.853 | 1.00 | 42.44 | O |
| ATOM | 3838 | OD2 | ASP | C | 207 | 23.729 | −27.293 | −8.246 | 1.00 | 42.93 | O |
| ATOM | 3839 | C | ASP | C | 207 | 22.504 | −26.068 | −12.785 | 1.00 | 42.04 | C |
| ATOM | 3840 | O | ASP | C | 207 | 23.559 | −26.381 | −13.316 | 1.00 | 43.34 | O |
| ATOM | 3841 | N | ASP | C | 208 | 21.674 | −25.181 | −13.308 | 1.00 | 45.29 | N |
| ATOM | 3842 | CA | ASP | C | 208 | 21.960 | −24.531 | −14.583 | 1.00 | 48.37 | C |
| ATOM | 3843 | CB | ASP | C | 208 | 20.741 | −23.737 | −15.080 | 1.00 | 49.23 | C |
| ATOM | 3844 | CG | ASP | C | 208 | 20.868 | −23.297 | −16.519 | 1.00 | 48.43 | C |
| ATOM | 3845 | OD1 | ASP | C | 208 | 21.688 | −22.402 | −16.794 | 1.00 | 47.33 | O |
| ATOM | 3846 | OD2 | ASP | C | 208 | 20.145 | −23.843 | −17.376 | 1.00 | 48.68 | O |
| ATOM | 3847 | C | ASP | C | 208 | 23.164 | −23.604 | −14.416 | 1.00 | 51.87 | C |
| ATOM | 3848 | O | ASP | C | 208 | 23.207 | −22.797 | −13.498 | 1.00 | 50.74 | O |
| ATOM | 3849 | N | ARG | C | 209 | 24.137 | −23.747 | −15.305 | 1.00 | 57.47 | N |
| ATOM | 3850 | CA | ARG | C | 209 | 25.399 | −22.995 | −15.238 | 1.00 | 60.88 | C |
| ATOM | 3851 | CB | ARG | C | 209 | 26.440 | −23.628 | −16.190 | 1.00 | 64.02 | C |
| ATOM | 3852 | CG | ARG | C | 209 | 26.156 | −23.452 | −17.677 | 1.00 | 66.54 | C |
| ATOM | 3853 | CD | ARG | C | 209 | 26.454 | −24.708 | −18.488 | 1.00 | 68.56 | C |
| ATOM | 3854 | NE | ARG | C | 209 | 26.494 | −24.457 | −19.928 | 1.00 | 70.64 | N |
| ATOM | 3855 | CZ | ARG | C | 209 | 25.434 | −24.320 | −20.736 | 1.00 | 72.40 | C |
| ATOM | 3856 | NH1 | ARG | C | 209 | 24.183 | −24.378 | −20.275 | 1.00 | 72.34 | N |
| ATOM | 3857 | NH2 | ARG | C | 209 | 25.631 | −24.111 | −22.042 | 1.00 | 73.64 | N |
| ATOM | 3858 | C | ARG | C | 209 | 25.268 | −21.488 | −15.516 | 1.00 | 62.34 | C |
| ATOM | 3859 | O | ARG | C | 209 | 26.117 | −20.726 | −15.072 | 1.00 | 65.86 | O |
| ATOM | 3860 | N | ASN | C | 210 | 24.223 | −21.065 | −16.226 | 1.00 | 62.40 | N |
| ATOM | 3861 | CA | ASN | C | 210 | 23.993 | −19.636 | −16.522 | 1.00 | 61.01 | C |
| ATOM | 3862 | CB | ASN | C | 210 | 23.530 | −19.451 | −17.972 | 1.00 | 61.71 | C |
| ATOM | 3863 | CG | ASN | C | 210 | 24.490 | −20.054 | −18.979 | 1.00 | 63.15 | C |
| ATOM | 3864 | OD1 | ASN | C | 210 | 24.075 | −20.582 | −20.011 | 1.00 | 63.33 | O |
| ATOM | 3865 | ND2 | ASN | C | 210 | 25.779 | −19.970 | −18.688 | 1.00 | 62.41 | N |
| ATOM | 3866 | C | ASN | C | 210 | 22.987 | −18.976 | −15.574 | 1.00 | 60.48 | C |
| ATOM | 3867 | O | ASN | C | 210 | 23.236 | −17.874 | −15.090 | 1.00 | 62.46 | O |
| ATOM | 3868 | N | THR | C | 211 | 21.855 | −19.641 | −15.328 | 1.00 | 58.17 | N |
| ATOM | 3869 | CA | THR | C | 211 | 20.770 | −19.087 | −14.496 | 1.00 | 54.93 | C |
| ATOM | 3870 | CB | THR | C | 211 | 19.383 | −19.494 | −15.038 | 1.00 | 55.36 | C |
| ATOM | 3871 | OG1 | THR | C | 211 | 19.234 | −20.913 | −14.957 | 1.00 | 55.13 | O |
| ATOM | 3872 | CG2 | THR | C | 211 | 19.201 | −19.057 | −16.487 | 1.00 | 55.94 | C |
| ATOM | 3873 | C | THR | C | 211 | 20.824 | −19.507 | −13.026 | 1.00 | 52.70 | C |
| ATOM | 3874 | O | THR | C | 211 | 20.152 | −18.899 | −12.196 | 1.00 | 50.96 | O |
| ATOM | 3875 | N | PHE | C | 212 | 21.586 | −20.563 | −12.721 | 1.00 | 52.14 | N |
| ATOM | 3876 | CA | PHE | C | 212 | 21.687 | −21.154 | −11.366 | 1.00 | 52.33 | C |
| ATOM | 3877 | CB | PHE | C | 212 | 22.210 | −20.142 | −10.333 | 1.00 | 54.37 | C |
| ATOM | 3878 | CG | PHE | C | 212 | 23.405 | −19.355 | −10.807 | 1.00 | 56.53 | C |
| ATOM | 3879 | CD1 | PHE | C | 212 | 24.617 | −19.994 | −11.061 | 1.00 | 57.27 | C |
| ATOM | 3880 | CE1 | PHE | C | 212 | 25.713 | −19.283 | −11.524 | 1.00 | 57.86 | C |
| ATOM | 3881 | CZ | PHE | C | 212 | 25.619 | −17.909 | −11.712 | 1.00 | 58.46 | C |
| ATOM | 3882 | CE2 | PHE | C | 212 | 24.417 | −17.257 | −11.466 | 1.00 | 58.43 | C |
| ATOM | 3883 | CD2 | PHE | C | 212 | 23.319 | −17.977 | −11.019 | 1.00 | 57.45 | C |
| ATOM | 3884 | C | PHE | C | 212 | 20.392 | −21.808 | −10.864 | 1.00 | 49.05 | C |
| ATOM | 3885 | O | PHE | C | 212 | 20.278 | −22.170 | −9.680 | 1.00 | 48.65 | O |
| ATOM | 3886 | N | ARG | C | 213 | 19.416 | −21.991 | −11.755 | 1.00 | 44.66 | N |
| ATOM | 3887 | CA | ARG | C | 213 | 18.130 | −22.565 | −11.372 | 1.00 | 43.54 | C |
| ATOM | 3888 | CB | ARG | C | 213 | 16.999 | −22.073 | −12.284 | 1.00 | 44.62 | C |
| ATOM | 3889 | CG | ARG | C | 213 | 16.692 | −20.609 | −12.045 | 1.00 | 46.29 | C |
| ATOM | 3890 | CD | ARG | C | 213 | 15.620 | −20.084 | −12.937 | 1.00 | 46.74 | C |
| ATOM | 3891 | NE | ARG | C | 213 | 15.422 | −18.635 | −12.797 | 1.00 | 48.21 | N |
| ATOM | 3892 | CZ | ARG | C | 213 | 14.670 | −18.033 | −11.872 | 1.00 | 47.73 | C |
| ATOM | 3893 | NH1 | ARG | C | 213 | 14.001 | −18.727 | −10.936 | 1.00 | 46.28 | N |
| ATOM | 3894 | NH2 | ARG | C | 213 | 14.583 | −16.702 | −11.904 | 1.00 | 47.36 | N |
| ATOM | 3895 | C | ARG | C | 213 | 18.211 | −24.079 | −11.349 | 1.00 | 39.93 | C |
| ATOM | 3896 | O | ARG | C | 213 | 19.011 | −24.694 | −12.047 | 1.00 | 36.63 | O |
| ATOM | 3897 | N | HIS | C | 214 | 17.370 | −24.662 | −10.505 | 1.00 | 38.28 | N |
| ATOM | 3898 | CA | HIS | C | 214 | 17.372 | −26.091 | −10.228 | 1.00 | 36.71 | C |
| ATOM | 3899 | CB | HIS | C | 214 | 17.148 | −26.335 | −8.739 | 1.00 | 37.32 | C |
| ATOM | 3900 | CG | HIS | C | 214 | 18.205 | −25.735 | −7.863 | 1.00 | 40.22 | C |
| ATOM | 3901 | ND1 | HIS | C | 214 | 17.985 | −25.442 | −6.538 | 1.00 | 41.91 | N |
| ATOM | 3902 | CE1 | HIS | C | 214 | 19.082 | −24.919 | −6.019 | 1.00 | 41.55 | C |
| ATOM | 3903 | NE2 | HIS | C | 214 | 20.003 | −24.847 | −6.965 | 1.00 | 41.86 | N |
| ATOM | 3904 | CD2 | HIS | C | 214 | 19.478 | −25.350 | −8.129 | 1.00 | 41.03 | C |
| ATOM | 3905 | C | HIS | C | 214 | 16.279 | −26.763 | −11.029 | 1.00 | 33.67 | C |
| ATOM | 3906 | O | HIS | C | 214 | 15.195 | −26.215 | −11.180 | 1.00 | 34.03 | O |
| ATOM | 3907 | N | SER | C | 215 | 16.572 | −27.942 | −11.555 | 1.00 | 30.90 | N |
| ATOM | 3908 | CA | SER | C | 215 | 15.552 | −28.742 | −12.210 | 1.00 | 29.34 | C |
| ATOM | 3909 | CB | SER | C | 215 | 15.425 | −28.345 | −13.684 | 1.00 | 28.69 | C |
| ATOM | 3910 | OG | SER | C | 215 | 16.611 | −28.602 | −14.403 | 1.00 | 28.61 | O |
| ATOM | 3911 | C | SER | C | 215 | 15.825 | −30.230 | −12.059 | 1.00 | 27.70 | C |
| ATOM | 3912 | O | SER | C | 215 | 16.936 | −30.637 | −11.733 | 1.00 | 27.10 | O |
| ATOM | 3913 | N | VAL | C | 216 | 14.787 | −31.019 | −12.304 | 1.00 | 27.09 | N |
| ATOM | 3914 | CA | VAL | C | 216 | 14.868 | −32.474 | −12.246 | 1.00 | 25.82 | C |
| ATOM | 3915 | CB | VAL | C | 216 | 14.211 | −33.036 | −10.958 | 1.00 | 24.98 | C |
| ATOM | 3916 | CG1 | VAL | C | 216 | 12.748 | −32.617 | −10.826 | 1.00 | 24.77 | C |
| ATOM | 3917 | CG2 | VAL | C | 216 | 14.340 | −34.554 | −10.904 | 1.00 | 24.76 | C |
| ATOM | 3918 | C | VAL | C | 216 | 14.228 | −33.034 | −13.513 | 1.00 | 25.60 | C |
| ATOM | 3919 | O | VAL | C | 216 | 13.133 | −32.613 | −13.899 | 1.00 | 25.87 | O |
| ATOM | 3920 | N | VAL | C | 217 | 14.920 | −33.970 | −14.158 | 1.00 | 25.19 | N |
| ATOM | 3921 | CA | VAL | C | 217 | 14.481 | −34.501 | −15.443 | 1.00 | 24.26 | C |
| ATOM | 3922 | CB | VAL | C | 217 | 15.329 | −33.926 | −16.610 | 1.00 | 24.09 | C |
| ATOM | 3923 | CG1 | VAL | C | 217 | 16.808 | −34.252 | −16.471 | 1.00 | 24.13 | C |
| ATOM | 3924 | CG2 | VAL | C | 217 | 14.822 | −34.423 | −34.423 | 1.00 | 24.12 | C |
| ATOM | 3925 | C | VAL | C | 217 | 14.455 | −36.037 | −15.411 | 1.00 | 23.44 | C |
| ATOM | 3926 | O | VAL | C | 217 | 15.300 | −36.662 | −14.771 | 1.00 | 24.23 | O |
| ATOM | 3927 | N | VAL | C | 218 | 13.446 | −36.623 | −16.061 | 1.00 | 23.07 | N |
| ATOM | 3928 | CA | VAL | C | 218 | 13.335 | −38.077 | −16.231 | 1.00 | 22.80 | C |
| ATOM | 3929 | CB | VAL | C | 218 | 12.250 | −38.724 | −15.320 | 1.00 | 22.45 | C |
| ATOM | 3930 | CG1 | VAL | C | 218 | 12.688 | −38.691 | −13.873 | 1.00 | 22.72 | C |
| ATOM | 3931 | CG2 | VAL | C | 218 | 10.881 | −38.072 | −15.497 | 1.00 | 22.70 | C |
| ATOM | 3932 | C | VAL | C | 218 | 12.997 | −38.390 | −17.689 | 1.00 | 22.93 | C |
| ATOM | 3933 | O | VAL | C | 218 | 12.505 | −37.508 | −18.403 | 1.00 | 21.24 | O |
| ATOM | 3934 | N | PRO | C | 219 | 13.256 | −39.639 | −18.132 | 1.00 | 23.67 | N |
| ATOM | 3935 | CA | PRO | C | 219 | 12.801 | −40.026 | −19.461 | 1.00 | 24.40 | C |
| ATOM | 3936 | CB | PRO | C | 219 | 13.381 | −41.440 | −19.644 | 1.00 | 23.80 | C |
| ATOM | 3937 | CG | PRO | C | 219 | 14.523 | −41.507 | −18.716 | 1.00 | 23.88 | C |
| ATOM | 3938 | CD | PRO | C | 219 | 14.123 | −40.673 | −17.537 | 1.00 | 24.11 | C |
| ATOM | 3939 | C | PRO | C | 219 | 11.280 | −40.059 | −19.559 | 1.00 | 24.92 | C |
| ATOM | 3940 | O | PRO | C | 219 | 10.619 | −40.560 | −18.655 | 1.00 | 26.56 | O |
| ATOM | 3941 | N | TYR | C | 220 | 10.745 | −39.510 | −20.643 | 1.00 | 25.40 | N |
| ATOM | 3942 | CA | TYR | C | 220 | 9.342 | −39.709 | −20.984 | 1.00 | 26.75 | C |
| ATOM | 3943 | CB | TYR | C | 220 | 8.917 | −38.812 | −22.148 | 1.00 | 26.95 | C |
| ATOM | 3944 | CG | TYR | C | 220 | 7.512 | −39.100 | −22.628 | 1.00 | 27.39 | C |
| ATOM | 3945 | CD1 | TYR | C | 220 | 6.411 | −38.568 | −21.952 | 1.00 | 27.89 | C |
| ATOM | 3946 | CE1 | TYR | C | 220 | 5.119 | −38.833 | −22.391 | 1.00 | 27.87 | C |
| ATOM | 3947 | CZ | TYR | C | 220 | 4.918 | −39.676 | −23.484 | 1.00 | 28.05 | C |
| ATOM | 3948 | OH | TYR | C | 220 | 3.626 | −39.939 | −23.896 | 1.00 | 29.41 | O |
| ATOM | 3949 | CE2 | TYR | C | 220 | 5.990 | −40.224 | −24.154 | 1.00 | 27.49 | C |
| ATOM | 3950 | CD2 | TYR | C | 220 | 7.279 | −39.938 | −23.732 | 1.00 | 26.73 | C |
| ATOM | 3951 | C | TYR | C | 220 | 9.151 | −41.163 | −21.382 | 1.00 | 27.71 | C |
| ATOM | 3952 | O | TYR | C | 220 | 9.795 | −41.645 | −22.314 | 1.00 | 26.89 | O |
| ATOM | 3953 | N | GLU | C | 221 | 8.278 | −41.859 | −20.670 | 1.00 | 28.96 | N |
| ATOM | 3954 | CA | GLU | C | 221 | 7.802 | −43.187 | −21.084 | 1.00 | 30.47 | C |
| ATOM | 3955 | CB | GLU | C | 221 | 7.916 | −44.176 | −19.913 | 1.00 | 32.48 | C |
| ATOM | 3956 | CG | GLU | C | 221 | 9.343 | −44.607 | −19.579 | 1.00 | 33.95 | C |
| ATOM | 3957 | CD | GLU | C | 221 | 10.029 | −45.404 | −20.684 | 1.00 | 36.73 | C |
| ATOM | 3958 | OE1 | GLU | C | 221 | 9.340 | −46.026 | −21.527 | 1.00 | 39.60 | O |
| ATOM | 3959 | OE2 | GLU | C | 221 | 11.284 | −45.418 | −20.701 | 1.00 | 38.01 | O |
| ATOM | 3960 | C | GLU | C | 221 | 6.346 | −43.024 | −21.518 | 1.00 | 30.63 | C |
| ATOM | 3961 | O | GLU | C | 221 | 5.609 | −42.274 | −20.888 | 1.00 | 28.62 | O |
| ATOM | 3962 | N | PRO | C | 222 | 5.914 | −43.690 | −22.602 | 1.00 | 32.01 | N |
| ATOM | 3963 | CA | PRO | C | 222 | 4.500 | −43.593 | −22.975 | 1.00 | 33.17 | C |
| ATOM | 3964 | CB | PRO | C | 222 | 4.437 | −44.326 | −24.328 | 1.00 | 32.80 | C |
| ATOM | 3965 | CG | PRO | C | 222 | 5.657 | −45.176 | −24.400 | 1.00 | 32.21 | C |
| ATOM | 3966 | CD | PRO | C | 222 | 6.691 | −44.472 | −23.592 | 1.00 | 32.15 | C |
| ATOM | 3967 | C | PRO | C | 222 | 3.562 | −44.297 | −21.935 | 1.00 | 33.65 | C |
| ATOM | 3968 | O | PRO | C | 222 | 4.053 | −45.074 | −21.108 | 1.00 | 34.07 | O |
| ATOM | 3969 | N | PRO | C | 223 | 2.256 | −43.987 | −21.974 | 1.00 | 34.39 | N |
| ATOM | 3970 | CA | PRO | C | 223 | 1.298 | −44.411 | −20.962 | 1.00 | 35.41 | C |
| ATOM | 3971 | CB | PRO | C | 223 | −0.079 | −44.155 | −21.608 | 1.00 | 35.07 | C |
| ATOM | 3972 | CG | PRO | C | 223 | 0.182 | −42.955 | −22.448 | 1.00 | 34.78 | C |
| ATOM | 3973 | CD | PRO | C | 223 | 1.623 | −43.035 | −22.888 | 1.00 | 34.03 | C |
| ATOM | 3974 | C | PRO | C | 223 | 1.351 | −45.795 | −20.464 | 1.00 | 36.31 | C |
| ATOM | 3975 | O | PRO | C | 223 | 1.477 | −46.669 | −21.275 | 1.00 | 33.77 | O |
| ATOM | 3976 | N | GLU | C | 224 | 1.239 | −45.984 | −19.139 | 1.00 | 39.09 | N |
| ATOM | 3977 | CA | GLU | C | 224 | 1.282 | −47.336 | −18.567 | 1.00 | 41.32 | C |
| ATOM | 3978 | CB | GLU | C | 224 | 1.481 | −47.305 | −17.042 | 1.00 | 43.73 | C |
| ATOM | 3979 | CG | GLU | C | 224 | 2.130 | −48.540 | −16.375 | 1.00 | 46.48 | C |
| ATOM | 3980 | CD | GLU | C | 224 | 3.418 | −49.030 | −17.052 | 1.00 | 48.99 | C |
| ATOM | 3981 | OE1 | GLU | C | 224 | 3.639 | −50.265 | −17.158 | 1.00 | 50.51 | O |
| ATOM | 3982 | OE2 | GLU | C | 224 | 4.164 | −48.152 | −17.532 | 1.00 | 48.26 | O |
| ATOM | 3983 | C | GLU | C | 224 | −0.021 | −48.049 | −18.926 | 1.00 | 41.61 | C |
| ATOM | 3984 | O | GLU | C | 224 | −1.002 | −47.414 | −19.345 | 1.00 | 40.79 | O |
| ATOM | 3985 | N | VAL | C | 225 | −0.008 | −49.363 | −18.734 | 1.00 | 43.00 | N |
| ATOM | 3986 | CA | VAL | C | 225 | −1.141 | −50.197 | −19.130 | 1.00 | 43.67 | C |
| ATOM | 3987 | CB | VAL | C | 225 | −0.823 | −51.730 | −19.124 | 1.00 | 44.17 | C |
| ATOM | 3988 | CG1 | VAL | C | 225 | 0.670 | −52.023 | −19.112 | 1.00 | 44.42 | C |
| ATOM | 3989 | CG2 | VAL | C | 225 | −1.517 | −52.481 | −17.978 | 1.00 | 43.96 | C |
| ATOM | 3990 | C | VAL | C | 225 | −2.305 | −49.849 | −18.206 | 1.00 | 43.34 | C |
| ATOM | 3991 | O | VAL | C | 225 | −2.146 | −49.815 | −16.981 | 1.00 | 43.35 | O |
| ATOM | 3992 | N | GLY | C | 226 | −3.463 | −49.552 | −18.803 | 1.00 | 43.84 | N |
| ATOM | 3993 | CA | GLY | C | 226 | −4.642 | −49.095 | −18.060 | 1.00 | 44.45 | C |
| ATOM | 3994 | C | GLY | C | 226 | −4.874 | −47.591 | −18.127 | 1.00 | 43.83 | C |
| ATOM | 3995 | O | GLY | C | 226 | −6.015 | −47.139 | −17.942 | 1.00 | 47.58 | O |
| ATOM | 3996 | N | SER | C | 227 | −3.814 | −46.811 | −18.335 | 1.00 | 41.32 | N |
| ATOM | 3997 | CA | SER | C | 227 | −3.895 | −45.349 | −18.334 | 1.00 | 39.91 | C |
| ATOM | 3998 | CB | SER | C | 227 | −2.712 | −44.789 | −17.529 | 1.00 | 39.40 | C |
| ATOM | 3999 | OG | SER | C | 227 | −2.946 | −43.409 | −17.311 | 1.00 | 38.42 | O |
| ATOM | 4000 | C | SER | C | 227 | −3.883 | −44.807 | −19.757 | 1.00 | 38.43 | C |
| ATOM | 4001 | O | SER | C | 227 | −3.365 | −45.458 | −20.673 | 1.00 | 36.36 | O |
| ATOM | 4002 | N | ASP | C | 228 | −4.374 | −43.578 | −19.929 | 1.00 | 38.32 | N |
| ATOM | 4003 | CA | ASP | C | 228 | −4.237 | −42.819 | −21.188 | 1.00 | 37.48 | C |
| ATOM | 4004 | CB | ASP | C | 228 | −5.606 | −42.282 | −21.612 | 1.00 | 40.71 | C |
| ATOM | 4005 | CG | ASP | C | 228 | −6.559 | −43.384 | −22.090 | 1.00 | 43.66 | C |
| ATOM | 4006 | OD1 | ASP | C | 228 | −6.124 | −44.537 | −22.352 | 1.00 | 46.86 | O |
| ATOM | 4007 | OD2 | ASP | C | 228 | −7.762 | −43.088 | −22.180 | 1.00 | 45.71 | O |
| ATOM | 4008 | C | ASP | C | 228 | −3.231 | −41.650 | −21.091 | 1.00 | 34.54 | C |
| ATOM | 4009 | O | ASP | C | 228 | −3.159 | −40.751 | −21.900 | 1.00 | 34.57 | O |
| ATOM | 4010 | N | CYS | C | 229 | −2.416 | −41.664 | −20.059 | 1.00 | 32.29 | N |
| ATOM | 4011 | CA | CYS | C | 229 | −1.416 | −40.680 | −19.709 | 1.00 | 31.19 | C |
| ATOM | 4012 | CB | CYS | C | 229 | −2.076 | −39.683 | −18.774 | 1.00 | 30.14 | C |
| ATOM | 4013 | SG | CYS | C | 229 | −2.743 | −40.353 | −17.224 | 1.00 | 27.88 | S |
| ATOM | 4014 | C | CYS | C | 229 | −0.243 | −41.334 | −18.961 | 1.00 | 30.26 | C |
| ATOM | 4015 | O | CYS | C | 229 | −0.320 | −42.494 | −18.495 | 1.00 | 31.63 | O |
| ATOM | 4016 | N | THR | C | 230 | 0.841 | −40.570 | −18.831 | 1.00 | 28.51 | O |
| ATOM | 4017 | CA | THR | C | 230 | 2.019 | −40.960 | −18.070 | 1.00 | 27.88 | C |
| ATOM | 4018 | CB | THR | C | 230 | 3.303 | −40.614 | −18.847 | 1.00 | 28.10 | C |
| ATOM | 4019 | OG1 | THR | C | 230 | 3.245 | −41.244 | −20.120 | 1.00 | 28.44 | O |
| ATOM | 4020 | CG2 | THR | C | 230 | 4.557 | −41.089 | −18.106 | 1.00 | 27.98 | C |
| ATOM | 4021 | C | THR | C | 230 | 2.046 | −40.201 | −16.749 | 1.00 | 27.26 | C |
| ATOM | 4022 | O | THR | C | 230 | 1.890 | −38.987 | −16.747 | 1.00 | 27.96 | O |
| ATOM | 4023 | N | THR | C | 231 | 2.294 | −40.917 | −15.657 | 1.00 | 26.34 | N |
| ATOM | 4024 | CA | THR | C | 231 | 2.285 | −40.348 | −14.308 | 1.00 | 26.58 | C |
| ATOM | 4025 | CB | THR | C | 231 | 1.349 | −41.153 | −13.377 | 1.00 | 26.23 | C |
| ATOM | 4026 | OG1 | THR | C | 231 | 0.033 | −41.204 | −13.946 | 1.00 | 26.16 | O |
| ATOM | 4027 | CG2 | THR | C | 231 | 1.271 | −40.527 | −11.998 | 1.00 | 26.22 | C |
| ATOM | 4028 | C | THR | C | 231 | 3.688 | −40.314 | −13.706 | 1.00 | 26.29 | C |
| ATOM | 4029 | O | THR | C | 231 | 4.344 | −41.352 | −13.601 | 1.00 | 27.78 | O |
| ATOM | 4030 | N | ILE | C | 232 | 4.140 | −39.119 | −13.316 | 1.00 | 26.14 | N |
| ATOM | 4031 | CA | ILE | C | 232 | 5.387 | −38.943 | −12.573 | 1.00 | 26.28 | C |
| ATOM | 4032 | CB | ILE | C | 232 | 6.260 | −37.827 | −13.206 | 1.00 | 26.16 | C |
| ATOM | 4033 | CG1 | ILE | C | 232 | 6.694 | −38.235 | −14.620 | 1.00 | 26.67 | C |
| ATOM | 4034 | CD1 | ILE | C | 232 | 7.274 | −37.105 | −15.419 | 1.00 | 26.81 | C |
| ATOM | 4035 | CG2 | ILE | C | 232 | 7.500 | −37.530 | −12.334 | 1.00 | 25.87 | C |
| ATOM | 4036 | C | ILE | C | 232 | 5.049 | −38.591 | −11.133 | 1.00 | 26.69 | C |
| ATOM | 4037 | O | ILE | C | 232 | 4.102 | −37.850 | −10.883 | 1.00 | 26.80 | O |
| ATOM | 4038 | N | HIS | C | 233 | 5.822 | −39.137 | −10.191 | 1.00 | 28.25 | O |
| ATOM | 4039 | CA | HIS | C | 233 | 5.642 | −38.878 | −8.764 | 1.00 | 27.95 | C |
| ATOM | 4040 | CB | HIS | C | 233 | 5.744 | −40.192 | −7.982 | 1.00 | 30.04 | C |
| ATOM | 4041 | CG | HIS | C | 233 | 4.545 | −41.083 | −8.132 | 1.00 | 31.71 | C |
| ATOM | 4042 | ND1 | HIS | C | 233 | 4.566 | −42.418 | −7.795 | 1.00 | 33.89 | N |
| ATOM | 4043 | CE1 | HIS | C | 233 | 3.376 | −42.945 | −8.028 | 1.00 | 34.79 | C |
| ATOM | 4044 | NE2 | HIS | C | 233 | 2.588 | −42.002 | −8.512 | 1.00 | 34.18 | N |
| ATOM | 4045 | CD2 | HIS | C | 233 | 3.296 | −40.831 | −8.593 | 1.00 | 33.01 | C |
| ATOM | 4046 | C | HIS | C | 233 | 6.685 | −37.897 | −8.265 | 1.00 | 27.17 | C |
| ATOM | 4047 | O | HIS | C | 233 | 7.845 | −38.269 | −8.062 | 1.00 | 27.40 | O |
| ATOM | 4048 | N | TYR | C | 234 | 6.275 | −36.648 | −8.067 | 1.00 | 26.31 | N |
| ATOM | 4049 | CA | TYR | C | 234 | 7.148 | −35.607 | −7.515 | 1.00 | 25.88 | C |
| ATOM | 4050 | CB | TYR | C | 234 | 6.800 | −34.236 | −8.121 | 1.00 | 24.61 | C |
| ATOM | 4051 | CG | TYR | C | 234 | 7.121 | −34.115 | −9.591 | 1.00 | 23.65 | C |
| ATOM | 4052 | CD1 | TYR | C | 234 | 8.446 | −34.017 | −10.025 | 1.00 | 22.93 | C |
| ATOM | 4053 | CE1 | TYR | C | 234 | 8.751 | −33.912 | −11.383 | 1.00 | 22.58 | C |
| ATOM | 4054 | CZ | TYR | C | 234 | 7.733 | −33.892 | −12.313 | 1.00 | 22.11 | C |
| ATOM | 4055 | OH | TYR | C | 234 | 8.036 | −33.777 | −13.647 | 1.00 | 22.34 | O |
| ATOM | 4056 | CE2 | TYR | C | 234 | 6.408 | −33.976 | −11.907 | 1.00 | 22.65 | C |
| ATOM | 4057 | CD2 | TYR | C | 234 | 6.103 | −34.086 | −10.560 | 1.00 | 22.75 | C |
| ATOM | 4058 | C | TYR | C | 234 | 7.033 | −35.548 | −5.988 | 1.00 | 26.99 | C |
| ATOM | 4059 | O | TYR | C | 234 | 6.011 | −35.944 | −5.421 | 1.00 | 26.42 | O |
| ATOM | 4060 | N | ASN | C | 235 | 8.085 | −35.058 | −5.336 | 1.00 | 29.06 | N |
| ATOM | 4061 | CA | ASN | C | 235 | 8.055 | −34.723 | −3.908 | 1.00 | 31.61 | C |
| ATOM | 4062 | CB | ASN | C | 235 | 8.985 | −35.631 | −3.085 | 1.00 | 31.98 | C |
| ATOM | 4063 | CG | ASN | C | 235 | 8.714 | −37.108 | −3.282 | 1.00 | 33.45 | C |
| ATOM | 4064 | OD1 | ASN | C | 235 | 9.649 | −37.901 | −3.257 | 1.00 | 37.01 | O |
| ATOM | 4065 | ND2 | ASN | C | 235 | 7.455 | −37.487 | −3.474 | 1.00 | 34.05 | N |
| ATOM | 4066 | C | ASN | C | 235 | 8.542 | −33.288 | −3.750 | 1.00 | 33.35 | C |
| ATOM | 4067 | O | ASN | C | 235 | 9.525 | −32.896 | −4.371 | 1.00 | 33.58 | O |
| ATOM | 4068 | N | TYR | C | 236 | 7.864 | −32.521 | −2.900 | 1.00 | 36.25 | N |
| ATOM | 4069 | CA | TYR | C | 236 | 8.285 | −31.166 | −2.550 | 1.00 | 39.55 | C |
| ATOM | 4070 | CB | TYR | C | 236 | 7.101 | −30.215 | −2.673 | 1.00 | 38.63 | C |
| ATOM | 4071 | CG | TYR | C | 236 | 6.642 | −30.032 | −4.111 | 1.00 | 36.87 | C |
| ATOM | 4072 | CD1 | TYR | C | 236 | 5.713 | −30.903 | −4.685 | 1.00 | 36.01 | C |
| ATOM | 4073 | CE1 | TYR | C | 236 | 5.290 | −30.739 | −5.995 | 1.00 | 35.95 | C |
| ATOM | 4074 | CZ | TYR | C | 236 | 5.797 | −29.689 | −6.759 | 1.00 | 35.63 | C |
| ATOM | 4075 | OH | TYR | C | 236 | 5.361 | −29.520 | −8.058 | 1.00 | 35.27 | O |
| ATOM | 4076 | CE2 | TYR | C | 236 | 6.713 | −28.812 | −6.208 | 1.00 | 34.76 | C |
| ATOM | 4077 | CD2 | TYR | C | 236 | 7.132 | −28.986 | −4.894 | 1.00 | 35.59 | C |
| ATOM | 4076 | C | TYR | C | 236 | 8.869 | −31.184 | −1.138 | 1.00 | 43.62 | C |
| ATOM | 4079 | O | TYR | C | 236 | 8.209 | −31.580 | −0.183 | 1.00 | 45.18 | O |
| ATOM | 4080 | N | MET | C | 237 | 10.111 | −30.719 | −1.021 | 1.00 | 48.98 | N |
| ATOM | 4081 | CA | MET | C | 237 | 10.958 | −30.914 | 0.166 | 1.00 | 54.77 | C |
| ATOM | 4082 | CB | MET | C | 237 | 12.312 | −31.451 | −0.263 | 1.00 | 54.02 | C |
| ATOM | 4083 | CG | MET | C | 237 | 12.253 | −32.708 | −1.161 | 1.00 | 54.11 | C |
| ATOM | 4084 | SD | MET | C | 237 | 11.435 | −34.150 | −0.448 | 1.00 | 55.08 | S |
| ATOM | 4085 | CE | MET | C | 237 | 12.415 | −34.347 | 1.036 | 1.00 | 56.41 | C |
| ATOM | 4086 | C | MET | C | 237 | 11.182 | −29.600 | 0.962 | 1.00 | 61.97 | C |
| ATOM | 4087 | O | MET | C | 237 | 12.251 | −29.340 | 1.556 | 1.00 | 62.58 | O |
| ATOM | 4088 | N | CYS | C | 238 | 10.141 | −28.770 | 1.038 | 1.00 | 71.32 | N |
| ATOM | 4089 | CA | CYS | C | 238 | 10.149 | −27.544 | 1.841 | 1.00 | 76.82 | C |
| ATOM | 4090 | CB | CYS | C | 238 | 10.651 | −26.391 | 0.982 | 1.00 | 78.32 | C |
| ATOM | 4091 | SG | CYS | C | 238 | 11.034 | −24.845 | 1.839 | 1.00 | 83.36 | S |
| ATOM | 4092 | C | CYS | C | 238 | 8.767 | −27.283 | 2.416 | 1.00 | 80.79 | C |
| ATOM | 4093 | O | CYS | C | 238 | 7.820 | −27.998 | 2.099 | 1.00 | 82.21 | O |
| ATOM | 4094 | N | ASN | C | 239 | 8.662 | −26.302 | 3.308 | 1.00 | 85.84 | N |
| ATOM | 4095 | CA | ASN | C | 239 | 7.392 | −25.873 | 3.918 | 1.00 | 88.89 | C |
| ATOM | 4096 | CB | ASN | C | 239 | 7.523 | −25.889 | 5.466 | 1.00 | 90.72 | C |
| ATOM | 4097 | CG | ASN | C | 239 | 6.243 | −26.306 | 6.145 | 1.00 | 91.65 | C |
| ATOM | 4098 | OD1 | ASN | C | 239 | 5.638 | −25.519 | 6.857 | 1.00 | 94.17 | O |
| ATOM | 4099 | ND2 | ASN | C | 239 | 5.798 | −27.529 | 5.885 | 1.00 | 91.17 | N |
| ATOM | 4100 | C | ASN | C | 239 | 7.088 | −24.470 | 3.351 | 1.00 | 89.54 | C |
| ATOM | 4101 | O | ASN | C | 239 | 8.008 | −23.672 | 3.249 | 1.00 | 91.33 | O |
| ATOM | 4102 | N | SER | C | 240 | 5.834 | −24.188 | 2.967 | 1.00 | 87.22 | N |
| ATOM | 4103 | CA | SER | C | 240 | 5.465 | −22.843 | 2.455 | 1.00 | 85.79 | C |
| ATOM | 4104 | CB | SER | C | 240 | 4.025 | −22.805 | 1.947 | 1.00 | 86.25 | C |
| ATOM | 4105 | OG | SER | C | 240 | 3.853 | −23.612 | 0.788 | 1.00 | 84.34 | O |
| ATOM | 4106 | C | SER | C | 240 | 5.626 | −21.714 | 3.475 | 1.00 | 84.14 | C |
| ATOM | 4107 | O | SER | C | 240 | 5.766 | −20.555 | 3.081 | 1.00 | 80.46 | O |
| ATOM | 4108 | N | SER | C | 241 | 5.539 | −22.055 | 4.765 | 1.00 | 84.16 | N |
| ATOM | 4109 | CA | SER | C | 241 | 5.852 | −21.138 | 5.861 | 1.00 | 84.05 | C |
| ATOM | 4110 | CB | SER | C | 241 | 5.062 | −21.519 | 7.118 | 1.00 | 83.40 | C |
| ATOM | 4111 | OG | SER | C | 241 | 5.365 | −22.846 | 7.524 | 1.00 | 82.24 | O |
| ATOM | 4112 | C | SER | C | 241 | 7.349 | −21.177 | 6.161 | 1.00 | 84.26 | C |
| ATOM | 4113 | O | SER | C | 241 | 7.988 | −22.231 | 6.077 | 1.00 | 81.92 | O |
| ATOM | 4114 | N | PRO | C | 250 | 4.106 | −17.150 | −3.569 | 1.00 | 55.57 | N |
| ATOM | 4115 | CA | PRO | C | 250 | 3.641 | −18.426 | −4.123 | 1.00 | 54.24 | C |
| ATOM | 4116 | CB | PRO | C | 250 | 2.327 | −18.055 | −4.824 | 1.00 | 56.06 | C |
| ATOM | 4117 | CG | PRO | C | 250 | 1.888 | −16.756 | −4.211 | 1.00 | 56.78 | C |
| ATOM | 4118 | CD | PRO | C | 250 | 3.159 | −16.048 | −3.823 | 1.00 | 57.54 | C |
| ATOM | 4119 | C | PRO | C | 250 | 4.632 | −19.028 | −5.119 | 1.00 | 58.69 | C |
| ATOM | 4120 | O | PRO | C | 250 | 5.300 | −18.281 | −5.856 | 1.00 | 50.53 | O |
| ATOM | 4121 | N | ILE | C | 251 | 4.725 | −20.366 | −5.117 | 1.00 | 47.80 | N |
| ATOM | 4122 | CA | ILE | C | 251 | 5.661 | −21.072 | −5.997 | 1.00 | 46.33 | C |
| ATOM | 4123 | CB | ILE | C | 251 | 6.423 | −22.171 | −5.240 | 1.00 | 46.62 | C |
| ATOM | 4124 | CG1 | ILE | C | 251 | 7.388 | −21.498 | −4.237 | 1.00 | 48.36 | C |
| ATOM | 4125 | CD1 | ILE | C | 251 | 7.865 | −22.411 | −3.117 | 1.00 | 49.28 | C |
| ATOM | 4126 | CG2 | ILE | C | 251 | 7.203 | −23.078 | −6.217 | 1.00 | 47.07 | C |
| ATOM | 4127 | C | ILE | C | 251 | 4.672 | −21.648 | −7.184 | 1.00 | 42.78 | C |
| ATOM | 4128 | O | ILE | C | 251 | 3.734 | −22.105 | −7.015 | 1.00 | 42.46 | O |
| ATOM | 4129 | N | LEU | C | 252 | 5.491 | −21.590 | −8.368 | 1.00 | 38.85 | N |
| ATOM | 4130 | CA | LEU | C | 252 | 4.937 | −22.144 | −9.591 | 1.00 | 36.71 | C |
| ATOM | 4131 | CB | LEU | C | 252 | 4.870 | −21.075 | −10.682 | 1.00 | 37.74 | C |
| ATOM | 4132 | CG | LEU | C | 252 | 3.989 | −19.872 | −10.320 | 1.00 | 40.24 | C |
| ATOM | 4133 | CD1 | LEU | C | 252 | 4.337 | −18.646 | −11.138 | 1.00 | 41.96 | C |
| ATOM | 4134 | CD2 | LEU | C | 252 | 2.510 | −20.222 | −10.441 | 1.00 | 40.61 | C |
| ATOM | 4135 | C | LEU | C | 252 | 5.859 | −23.254 | −10.049 | 1.00 | 32.82 | C |
| ATOM | 4136 | O | LEU | C | 252 | 7.057 | −23.046 | −10.115 | 1.00 | 30.55 | O |
| ATOM | 4137 | N | THR | C | 253 | 5.298 | −24.423 | −10.369 | 1.00 | 29.86 | N |
| ATOM | 4136 | CA | THR | C | 253 | 6.049 | −25.518 | −10.966 | 1.00 | 28.22 | C |
| ATOM | 4139 | CB | THR | C | 253 | 5.605 | −26.876 | −10.397 | 1.00 | 27.76 | C |
| ATOM | 4140 | OG1 | THR | C | 253 | 5.849 | −26.905 | −8.985 | 1.00 | 28.37 | O |
| ATOM | 4141 | CG2 | THR | C | 253 | 6.355 | −28.041 | −11.062 | 1.00 | 27.12 | C |
| ATOM | 4142 | C | THR | C | 253 | 5.842 | −25.492 | −12.483 | 1.00 | 27.20 | C |
| ATOM | 4143 | O | THR | C | 253 | 4.709 | −25.419 | −12.950 | 1.00 | 26.15 | O |
| ATOM | 4144 | N | ILE | C | 254 | 6.946 | −25.541 | −13.232 | 1.00 | 25.84 | N |
| ATOM | 4145 | CA | ILE | C | 254 | 6.929 | −25.562 | −14.685 | 1.00 | 25.32 | C |
| ATOM | 4146 | CB | ILE | C | 254 | 7.868 | −24.490 | −15.275 | 1.00 | 25.92 | C |
| ATOM | 4147 | CG1 | ILE | C | 254 | 7.389 | −23.085 | −14.888 | 1.00 | 27.07 | C |
| ATOM | 4148 | CD1 | ILE | C | 254 | 8.491 | −22.049 | −14.998 | 1.00 | 27.79 | C |
| ATOM | 4149 | CG2 | ILE | C | 254 | 7.988 | −24.623 | −16.796 | 1.00 | 25.67 | C |
| ATOM | 4150 | C | ILE | C | 254 | 7.380 | −26.928 | −15.167 | 1.00 | 24.45 | C |
| ATOM | 4151 | O | ILE | C | 254 | 8.509 | −27.327 | −14.904 | 1.00 | 24.77 | O |
| ATOM | 4152 | N | ILE | C | 255 | 6.492 | −27.622 | −15.879 | 1.00 | 23.34 | N |
| ATOM | 4153 | CA | ILE | C | 255 | 6.789 | −28.913 | −16.492 | 1.00 | 22.37 | C |
| ATOM | 4154 | CB | ILE | C | 255 | 5.646 | −29.934 | −16.274 | 1.00 | 21.99 | C |
| ATOM | 4155 | CG1 | ILE | C | 255 | 5.273 | −30.058 | −14.788 | 1.00 | 21.49 | C |
| ATOM | 4156 | CD1 | ILE | C | 255 | 6.401 | −30.464 | −13.861 | 1.00 | 21.22 | C |
| ATOM | 4157 | CG2 | ILE | C | 255 | 6.020 | −31.304 | −16.831 | 1.00 | 22.54 | C |
| ATOM | 4158 | C | ILE | C | 255 | 6.991 | −28.681 | −17.982 | 1.00 | 22.53 | C |
| ATOM | 4159 | O | ILE | C | 255 | 6.100 | −28.159 | −18.643 | 1.00 | 23.01 | O |
| ATOM | 4160 | N | THR | C | 256 | 8.162 | −29.049 | −18.495 | 1.00 | 22.66 | N |
| ATOM | 4161 | CA | THR | C | 256 | 8.461 | −28.949 | −19.917 | 1.00 | 22.00 | C |
| ATOM | 4162 | CB | THR | C | 256 | 9.669 | −28.027 | −20.194 | 1.00 | 22.30 | C |
| ATOM | 4163 | OG1 | THR | C | 256 | 10.777 | −28.417 | −19.392 | 1.00 | 22.90 | O |
| ATOM | 4164 | CG2 | THR | C | 256 | 9.348 | −26.577 | −19.883 | 1.00 | 22.41 | C |
| ATOM | 4165 | C | THR | C | 256 | 8.749 | −30.325 | −20.516 | 1.00 | 22.01 | C |
| ATOM | 4166 | O | THR | C | 256 | 9.311 | −31.198 | −19.864 | 1.00 | 20.92 | O |
| ATOM | 4167 | N | LEU | C | 257 | 8.343 | −30.500 | −21.768 | 1.00 | 23.46 | N |
| ATOM | 4168 | CA | LEU | C | 257 | 8.690 | −31.660 | −22.562 | 1.00 | 24.87 | C |
| ATOM | 4169 | CB | LEU | C | 257 | 7.453 | −32.095 | −23.359 | 1.00 | 25.48 | C |
| ATOM | 4170 | CG | LEU | C | 257 | 7.341 | −33.561 | −23.790 | 1.00 | 26.10 | C |
| ATOM | 4171 | CD1 | LEU | C | 257 | 7.231 | −34.480 | −22.591 | 1.00 | 26.34 | C |
| ATOM | 4172 | CD2 | LEU | C | 257 | 6.131 | −33.715 | −24.699 | 1.00 | 27.28 | C |
| ATOM | 4173 | C | LEU | C | 257 | 9.856 | −31.265 | −23.482 | 1.00 | 25.21 | C |
| ATOM | 4174 | O | LEU | C | 257 | 9.748 | −30.271 | −24.192 | 1.00 | 25.65 | O |
| ATOM | 4175 | N | GLU | C | 258 | 10.952 | −32.022 | −23.439 | 1.00 | 25.21 | N |
| ATOM | 4176 | CA | GLU | C | 258 | 12.133 | −31.804 | −24.305 | 1.00 | 25.92 | C |
| ATOM | 4177 | CB | GLU | C | 258 | 13.389 | −31.518 | −23.466 | 1.00 | 26.82 | C |
| ATOM | 4178 | CG | GLU | C | 258 | 13.245 | −30.515 | −22.345 | 1.00 | 28.38 | C |
| ATOM | 4179 | CD | GLU | C | 258 | 14.546 | −30.269 | −21.596 | 1.00 | 29.08 | C |
| ATOM | 4180 | OE1 | GLU | C | 258 | 15.427 | −31.152 | −21.547 | 1.00 | 29.92 | O |
| ATOM | 4181 | OE2 | GLU | C | 258 | 14.692 | −29.178 | −21.027 | 1.00 | 29.93 | O |
| ATOM | 4182 | C | GLU | C | 258 | 12.459 | −33.048 | −25.123 | 1.00 | 25.81 | C |
| ATOM | 4183 | O | GLU | C | 258 | 12.191 | −34.158 | −24.676 | 1.00 | 25.07 | O |
| ATOM | 4184 | N | ASP | C | 259 | 13.105 | −32.873 | −26.274 | 1.00 | 26.07 | N |
| ATOM | 4185 | CA | ASP | C | 259 | 13.687 | −34.018 | −27.014 | 1.00 | 26.37 | C |
| ATOM | 4186 | CB | ASP | C | 259 | 13.773 | −33.758 | −28.540 | 1.00 | 26.37 | C |
| ATOM | 4187 | CG | ASP | C | 259 | 14.800 | −32.693 | −28.934 | 1.00 | 27.32 | C |
| ATOM | 4188 | OD1 | ASP | C | 259 | 15.687 | −32.324 | −28.134 | 1.00 | 29.31 | O |
| ATOM | 4189 | OD2 | ASP | C | 259 | 14.715 | −32.224 | −30.090 | 1.00 | 25.79 | O |
| ATOM | 4190 | C | ASP | C | 259 | 15.036 | −34.433 | −26.409 | 1.00 | 26.31 | C |
| ATOM | 4191 | O | ASP | C | 259 | 15.491 | −33.822 | −25.439 | 1.00 | 26.75 | O |
| ATOM | 4192 | N | SER | C | 260 | 15.669 | −35.455 | −26.978 | 1.00 | 26.43 | N |
| ATOM | 4193 | CA | SER | C | 260 | 16.964 | −35.934 | −26.486 | 1.00 | 26.34 | C |
| ATOM | 4194 | CB | SER | C | 260 | 17.391 | −37.215 | −27.217 | 1.00 | 27.37 | C |
| ATOM | 4195 | OG | SER | C | 260 | 17.288 | −37.065 | −28.617 | 1.00 | 30.39 | O |
| ATOM | 4196 | C | SER | C | 260 | 18.078 | −34.891 | −26.564 | 1.00 | 25.47 | C |
| ATOM | 4197 | O | SER | C | 260 | 18.988 | −34.911 | −25.737 | 1.00 | 25.42 | O |
| ATOM | 4198 | N | SER | C | 261 | 17.996 | −33.976 | −27.532 | 1.00 | 24.93 | N |
| ATOM | 4199 | CA | SER | C | 261 | 18.981 | −32.898 | −27.700 | 1.00 | 24.34 | C |
| ATOM | 4200 | CB | SER | C | 261 | 19.154 | −32.564 | −29.183 | 1.00 | 24.67 | C |
| ATOM | 4201 | OG | SER | C | 261 | 19.379 | −33.736 | −29.954 | 1.00 | 25.31 | O |
| ATOM | 4202 | C | SER | C | 261 | 18.636 | −31.615 | −26.931 | 1.00 | 23.92 | C |
| ATOM | 4203 | O | SER | C | 261 | 19.315 | −30.600 | −27.103 | 1.00 | 23.79 | O |
| ATOM | 4204 | N | GLY | C | 262 | 17.592 | −31.646 | −26.096 | 1.00 | 23.18 | N |
| ATOM | 4205 | CA | GLY | C | 262 | 17.244 | −30.514 | −25.237 | 1.00 | 22.90 | C |
| ATOM | 4206 | C | GLY | C | 262 | 16.326 | −29.450 | −25.829 | 1.00 | 23.41 | C |
| ATOM | 4207 | O | GLY | C | 262 | 16.093 | −28.429 | −25.169 | 1.00 | 23.31 | O |
| ATOM | 4208 | N | ASN | C | 263 | 15.791 | −29.673 | −27.039 | 1.00 | 22.49 | N |
| ATOM | 4209 | CA | ASN | C | 263 | 14.875 | −28.718 | −27.662 | 1.00 | 22.79 | C |
| ATOM | 4210 | CB | ASN | C | 263 | 14.739 | −28.964 | −29.170 | 1.00 | 22.17 | C |
| ATOM | 4211 | CG | ASN | C | 263 | 16.056 | −28.829 | −29.911 | 1.00 | 21.70 | C |
| ATOM | 4212 | OD1 | ASN | C | 263 | 16.609 | −27.741 | −30.020 | 1.00 | 20.73 | O |
| ATOM | 4213 | ND2 | ASN | C | 263 | 16.551 | −29.939 | −30.442 | 1.00 | 21.36 | N |
| ATOM | 4214 | C | ASN | C | 263 | 13.488 | −28.806 | −27.019 | 1.00 | 23.57 | C |
| ATOM | 4215 | O | ASN | C | 263 | 12.993 | −29.901 | −26.762 | 1.00 | 23.07 | O |
| ATOM | 4216 | N | LEU | C | 264 | 12.866 | −27.652 | −26.800 | 1.00 | 24.67 | N |
| ATOM | 4217 | CA | LEU | C | 264 | 11.550 | −27.580 | −26.173 | 1.00 | 24.74 | C |
| ATOM | 4218 | CB | LEU | C | 264 | 11.271 | −26.172 | −25.633 | 1.00 | 26.05 | C |
| ATOM | 4219 | CG | LEU | C | 264 | 11.657 | −25.935 | −24.141 | 1.00 | 27.31 | C |
| ATOM | 4220 | CD1 | LEU | C | 264 | 13.139 | −26.129 | −23.829 | 1.00 | 27.10 | C |
| ATOM | 4221 | CD2 | LEU | C | 264 | 11.208 | −24.552 | −23.704 | 1.00 | 28.24 | C |
| ATOM | 4222 | C | LEU | C | 264 | 10.441 | −28.074 | −27.121 | 1.00 | 23.99 | C |
| ATOM | 4223 | O | LEU | C | 264 | 10.341 | −27.632 | −28.256 | 1.00 | 22.66 | O |
| ATOM | 4224 | N | LEU | C | 265 | 9.643 | −29.017 | −26.626 | 1.00 | 23.20 | N |
| ATOM | 4225 | CA | LEU | C | 265 | 8.473 | −29.530 | −27.329 | 1.00 | 23.27 | C |
| ATOM | 4226 | CB | LEU | C | 265 | 8.472 | −31.064 | −27.271 | 1.00 | 22.32 | C |
| ATOM | 4227 | CG | LEU | C | 265 | 9.764 | −31.765 | −27.694 | 1.00 | 21.65 | C |
| ATOM | 4228 | CD1 | LEU | C | 265 | 9.597 | −33.270 | −27.605 | 1.00 | 21.71 | C |
| ATOM | 4229 | CD2 | LEU | C | 265 | 10.173 | −31.334 | −29.092 | 1.00 | 21.54 | C |
| ATOM | 4230 | C | LEU | C | 265 | 7.156 | −28.990 | −26.769 | 1.00 | 23.45 | C |
| ATOM | 4231 | O | LEU | C | 265 | 6.188 | −28.831 | −27.521 | 1.00 | 24.55 | O |
| ATOM | 4232 | N | GLY | C | 266 | 7.111 | −28.717 | −25.467 | 1.00 | 22.38 | N |
| ATOM | 4233 | CA | GLY | C | 266 | 5.876 | −28.232 | −24.836 | 1.00 | 23.03 | C |
| ATOM | 4234 | C | GLY | C | 266 | 6.083 | −27.804 | −23.401 | 1.00 | 23.22 | C |
| ATOM | 4235 | O | GLY | C | 266 | 7.092 | −28.162 | −22.793 | 1.00 | 21.99 | O |
| ATOM | 4236 | N | ARG | C | 267 | 5.124 | −27.044 | −22.877 | 1.00 | 23.84 | N |
| ATOM | 4237 | CA | ARG | C | 267 | 5.215 | −26.480 | −21.534 | 1.00 | 24.78 | C |
| ATOM | 4238 | CB | ARG | C | 267 | 5.952 | −25.130 | −21.572 | 1.00 | 25.23 | C |
| ATOM | 4239 | CG | ARG | C | 267 | 6.302 | −24.593 | −20.180 | 1.00 | 25.56 | C |
| ATOM | 4240 | CD | ARG | C | 267 | 6.771 | −23.154 | −20.162 | 1.00 | 25.44 | C |
| ATOM | 4241 | NE | ARG | C | 267 | 8.026 | −22.921 | −20.873 | 1.00 | 25.82 | N |
| ATOM | 4242 | CZ | ARG | C | 267 | 8.161 | −22.294 | −22.053 | 1.00 | 25.28 | C |
| ATOM | 4243 | NH1 | ARG | C | 267 | 7.115 | −21.825 | −22.737 | 1.00 | 24.82 | N |
| ATOM | 4244 | NH2 | ARG | C | 267 | 9.375 | −22.137 | −22.567 | 1.00 | 26.00 | N |
| ATOM | 4245 | C | ARG | C | 267 | 3.832 | −26.268 | −20.909 | 1.00 | 25.74 | C |
| ATOM | 4246 | O | ARG | C | 267 | 2.879 | −25.877 | −21.586 | 1.00 | 26.75 | O |
| ATOM | 4247 | N | ASN | C | 268 | 3.742 | −26.542 | −19.609 | 1.00 | 25.34 | N |
| ATOM | 4248 | CA | ASN | C | 268 | 2.579 | −26.214 | −18.791 | 1.00 | 25.49 | C |
| ATOM | 4249 | CB | ASN | C | 268 | 1.628 | −27.410 | −18.724 | 1.00 | 27.70 | C |
| ATOM | 4250 | CG | ASN | C | 268 | 0.674 | −27.452 | −19.880 | 1.00 | 29.99 | C |
| ATOM | 4251 | OD1 | ASN | C | 268 | −0.222 | −26.626 | −19.951 | 1.00 | 31.06 | O |
| ATOM | 4252 | ND2 | ASN | C | 268 | 0.882 | −28.368 | −20.819 | 1.00 | 32.07 | N |
| ATOM | 4253 | C | ASN | C | 268 | 3.061 | −25.867 | −17.396 | 1.00 | 24.29 | C |
| ATOM | 4254 | O | ASN | C | 268 | 4.258 | −25.981 | −17.107 | 1.00 | 23.95 | O |
| ATOM | 4255 | N | SER | C | 269 | 2.144 | −25.433 | −16.536 | 1.00 | 22.77 | N |
| ATOM | 4256 | CA | SER | C | 269 | 2.512 | −25.039 | −15.195 | 1.00 | 22.85 | C |
| ATOM | 4257 | CB | SER | C | 269 | 3.151 | −23.646 | −15.217 | 1.00 | 22.14 | C |
| ATOM | 4258 | OG | SER | C | 269 | 2.244 | −22.687 | −15.722 | 1.00 | 21.22 | O |
| ATOM | 4259 | C | SER | C | 269 | 1.331 | −25.027 | −14.229 | 1.00 | 23.03 | C |
| ATOM | 4260 | O | SER | C | 269 | 0.163 | −24.997 | −14.639 | 1.00 | 23.11 | O |
| ATOM | 4261 | N | PHE | C | 270 | 1.668 | −25.050 | −12.944 | 1.00 | 23.04 | N |
| ATOM | 4262 | CA | PHE | C | 270 | 0.683 | −24.980 | −11.881 | 1.00 | 24.37 | C |
| ATOM | 4263 | CB | PHE | C | 270 | 0.054 | −26.354 | −11.612 | 1.00 | 24.27 | C |
| ATOM | 4264 | CG | PHE | C | 270 | 1.042 | −27.440 | −11.261 | 1.00 | 24.45 | C |
| ATOM | 4265 | CD1 | PHE | C | 270 | 1.714 | −28.147 | −12.266 | 1.00 | 24.90 | C |
| ATOM | 4266 | CE1 | PHE | C | 270 | 2.599 | −29.167 | −11.953 | 1.00 | 24.64 | C |
| ATOM | 4267 | CZ | PHE | C | 270 | 2.817 | −29.506 | −10.622 | 1.00 | 24.95 | C |
| ATOM | 4268 | CE2 | PHE | C | 270 | 2.157 | −28.818 | −9.613 | 1.00 | 24.74 | C |
| ATOM | 4269 | CD2 | PHE | C | 270 | 1.268 | −27.800 | −9.937 | 1.00 | 24.54 | C |
| ATOM | 4270 | C | PHE | C | 270 | 1.298 | −24.412 | −10.609 | 1.00 | 25.10 | C |
| ATOM | 4271 | O | PHE | C | 270 | 2.510 | −24.550 | −10.374 | 1.00 | 24.17 | O |
| ATOM | 4272 | N | GLU | C | 271 | 0.454 | −23.766 | −9.812 | 1.00 | 26.53 | N |
| ATOM | 4273 | CA | GLU | C | 271 | 0.856 | −23.223 | −8.533 | 1.00 | 28.77 | C |
| ATOM | 4274 | CB | GLU | C | 271 | −0.172 | −22.197 | −8.055 | 1.00 | 30.10 | C |
| ATOM | 4275 | CG | GLU | C | 271 | 0.262 | −21.347 | −6.873 | 1.00 | 31.75 | C |
| ATOM | 4276 | CD | GLU | C | 271 | −0.665 | −20.168 | −6.627 | 1.00 | 33.46 | C |
| ATOM | 4277 | OE1 | GLU | C | 271 | −1.196 | −19.570 | −7.599 | 1.00 | 36.07 | O |
| ATOM | 4278 | OE2 | GLU | C | 271 | −0.849 | −19.818 | −5.441 | 1.00 | 32.48 | O |
| ATOM | 4279 | C | GLU | C | 271 | 0.948 | −24.391 | −7.543 | 1.00 | 28.54 | C |
| ATOM | 4280 | O | GLU | C | 271 | 0.225 | −25.378 | −7.676 | 1.00 | 28.18 | O |
| ATOM | 4281 | N | VAL | C | 272 | 1.872 | −24.288 | −6.584 | 1.00 | 30.06 | N |
| ATOM | 4282 | CA | VAL | C | 272 | 2.019 | −25.319 | −5.564 | 1.00 | 33.04 | C |
| ATOM | 4283 | CB | VAL | C | 272 | 3.216 | −26.264 | −5.866 | 1.00 | 33.30 | C |
| ATOM | 4284 | CG1 | VAL | C | 272 | 3.319 | −27.412 | −4.850 | 1.00 | 32.57 | C |
| ATOM | 4285 | CG2 | VAL | C | 272 | 4.520 | −25.470 | −6.035 | 1.00 | 34.37 | C |
| ATOM | 4286 | C | VAL | C | 272 | 2.118 | −24.692 | −4.173 | 1.00 | 34.60 | C |
| ATOM | 4287 | O | VAL | C | 272 | 2.742 | −23.629 | −3.991 | 1.00 | 35.57 | O |
| ATOM | 4288 | N | ARG | C | 273 | 1.454 | −25.343 | −3.215 | 1.00 | 36.12 | N |
| ATOM | 4289 | CA | ARG | C | 273 | 1.506 | −24.976 | −1.809 | 1.00 | 37.28 | C |
| ATOM | 4290 | CB | ARG | C | 273 | 0.191 | −24.338 | −1.365 | 1.00 | 39.05 | C |
| ATOM | 4291 | CG | ARG | C | 273 | 0.109 | −24.044 | 0.123 | 1.00 | 41.44 | C |
| ATOM | 4292 | CD | ARG | C | 273 | −1.161 | −23.294 | 0.494 | 1.00 | 43.88 | C |
| ATOM | 4293 | NE | ARG | C | 273 | −1.002 | −22.654 | 1.811 | 1.00 | 46.23 | N |
| ATOM | 4294 | CZ | ARG | C | 273 | −0.277 | −21.554 | 2.055 | 1.00 | 48.54 | C |
| ATOM | 4295 | NH1 | ARG | C | 273 | 0.395 | −20.923 | 1.084 | 1.00 | 49.89 | N |
| ATOM | 4296 | NH2 | ARG | C | 273 | −0.216 | −21.080 | 3.295 | 1.00 | 48.11 | N |
| ATOM | 4297 | C | ARG | C | 273 | 1.763 | −26.246 | −1.003 | 1.00 | 36.02 | C |
| ATOM | 4298 | O | ARG | C | 273 | 0.980 | −27.201 | −1.059 | 1.00 | 33.12 | O |
| ATOM | 4299 | N | VAL | C | 274 | 2.860 | −26.240 | −0.247 | 1.00 | 36.32 | N |
| ATOM | 4300 | CA | VAL | C | 274 | 3.173 | −27.295 | 0.700 | 1.00 | 35.99 | C |
| ATOM | 4301 | CB | VAL | C | 274 | 4.692 | −27.542 | 0.782 | 1.00 | 35.79 | C |
| ATOM | 4302 | CG1 | VAL | C | 274 | 5.004 | −28.723 | 1.715 | 1.00 | 35.77 | C |
| ATOM | 4303 | CG2 | VAL | C | 274 | 5.231 | −27.831 | −0.615 | 1.00 | 35.83 | C |
| ATOM | 4304 | C | VAL | C | 274 | 2.596 | −26.875 | 2.048 | 1.00 | 36.18 | C |
| ATOM | 4305 | O | VAL | C | 274 | 2.857 | −25.770 | 2.506 | 1.00 | 36.36 | O |
| ATOM | 4306 | N | CYS | C | 275 | 1.799 | −27.751 | 2.659 | 1.00 | 37.94 | N |
| ATOM | 4307 | CA | CYS | C | 275 | 1.066 | −27.437 | 3.893 | 1.00 | 38.06 | C |
| ATOM | 4308 | CB | CYS | C | 275 | −0.189 | −25.623 | 3.552 | 1.00 | 38.84 | C |
| ATOM | 4309 | SG | CYS | C | 275 | −1.267 | −27.358 | 2.289 | 1.00 | 39.91 | S |
| ATOM | 4310 | C | CYS | C | 275 | 0.677 | −28.697 | 4.674 | 1.00 | 38.74 | C |
| ATOM | 4311 | O | CYS | C | 275 | 0.801 | −29.820 | 4.169 | 1.00 | 39.27 | O |
| ATOM | 4312 | N | ALA | C | 276 | 0.205 | −28.495 | 5.903 | 1.00 | 38.14 | N |
| ATOM | 4313 | CA | ALA | C | 276 | −0.128 | −29.596 | 6.816 | 1.00 | 37.13 | C |
| ATOM | 4314 | CB | ALA | C | 276 | −0.276 | −29.075 | 8.235 | 1.00 | 37.00 | C |
| ATOM | 4315 | C | ALA | C | 276 | −1.388 | −30.365 | 6.415 | 1.00 | 37.15 | C |
| ATOM | 4316 | O | ALA | C | 276 | −1.395 | −31.606 | 6.433 | 1.00 | 37.66 | O |
| ATOM | 4317 | N | CYS | C | 277 | −2.444 | −29.632 | 6.057 | 1.00 | 36.60 | N |
| ATOM | 4318 | CA | CYS | C | 277 | −3.739 | −30.222 | 5.703 | 1.00 | 36.93 | C |
| ATOM | 4319 | CB | CYS | C | 277 | −4.790 | −29.898 | 6.775 | 1.00 | 37.46 | C |
| ATOM | 4320 | SG | CYS | C | 277 | −6.198 | −31.026 | 6.741 | 1.00 | 39.57 | S |
| ATOM | 4321 | C | CYS | C | 277 | −4.205 | −29.740 | 4.316 | 1.00 | 34.69 | C |
| ATOM | 4322 | O | CYS | C | 277 | −4.978 | −28.781 | 4.224 | 1.00 | 33.58 | O |
| ATOM | 4323 | N | PRO | C | 278 | −3.732 | −30.399 | 3.235 | 1.00 | 35.31 | N |
| ATOM | 4324 | CA | PRO | C | 278 | −4.110 | −30.047 | 1.850 | 1.00 | 34.72 | C |
| ATOM | 4325 | CB | PRO | C | 278 | −3.517 | −31.189 | 1.021 | 1.00 | 33.66 | C |
| ATOM | 4326 | CG | PRO | C | 278 | −2.357 | −31.661 | 1.800 | 1.00 | 33.85 | C |
| ATOM | 4327 | CD | PRO | C | 278 | −2.668 | −31.421 | 3.256 | 1.00 | 34.26 | C |
| ATOM | 4328 | C | PRO | C | 278 | −5.617 | −29.945 | 1.587 | 1.00 | 35.03 | C |
| ATOM | 4329 | O | PRO | C | 278 | −6.069 | −28.964 | 0.988 | 1.00 | 35.80 | O |
| ATOM | 4330 | N | GLY | C | 279 | −6.369 | −30.951 | 2.034 | 1.00 | 35.82 | N |
| ATOM | 4331 | CA | GLY | C | 279 | −7.811 | −30.975 | 1.884 | 1.00 | 37.85 | C |
| ATOM | 4332 | C | GLY | C | 279 | −8.485 | −29.730 | 2.422 | 1.00 | 39.36 | C |
| ATOM | 4333 | O | GLY | C | 279 | −9.223 | −29.056 | 1.704 | 1.00 | 38.09 | O |
| ATOM | 4334 | N | ARG | C | 280 | −8.200 | −29.408 | 3.686 | 1.00 | 40.85 | N |
| ATOM | 4335 | CA | ARG | C | 280 | −8.809 | −28.259 | 4.353 | 1.00 | 43.41 | C |
| ATOM | 4336 | CB | ARG | C | 280 | −8.463 | −28.295 | 5.846 | 1.00 | 47.89 | C |
| ATOM | 4337 | CG | ARG | C | 280 | −8.775 | −27.079 | 6.726 | 1.00 | 51.90 | C |
| ATOM | 4338 | CD | ARG | C | 280 | −7.774 | −26.877 | 7.870 | 1.00 | 55.71 | C |
| ATOM | 4339 | NE | ARG | C | 280 | −6.401 | −26.526 | 7.449 | 1.00 | 57.67 | N |
| ATOM | 4340 | CZ | ARG | C | 280 | −5.349 | −26.409 | 8.259 | 1.00 | 60.22 | C |
| ATOM | 4341 | NH1 | ARG | C | 280 | −5.454 | −26.579 | 9.582 | 1.00 | 60.70 | N |
| ATOM | 4342 | NH2 | ARG | C | 280 | −4.155 | −26.104 | 7.747 | 1.00 | 61.41 | N |
| ATOM | 4343 | C | ARG | C | 280 | −8.376 | −26.947 | 3.695 | 1.00 | 41.99 | C |
| ATOM | 4344 | O | ARG | C | 280 | −9.222 | −26.089 | 3.427 | 1.00 | 41.30 | O |
| ATOM | 4345 | N | ASP | C | 281 | −7.074 | −26.808 | 3.440 | 1.00 | 40.50 | N |
| ATOM | 4346 | CA | ASP | C | 281 | −6.546 | −25.603 | 2.798 | 1.00 | 39.76 | C |
| ATOM | 4347 | CB | ASP | C | 281 | −5.000 | −25.604 | 2.799 | 1.00 | 41.59 | C |
| ATOM | 4348 | CG | ASP | C | 281 | −4.397 | −25.298 | 4.166 | 1.00 | 43.32 | C |
| ATOM | 4349 | OD1 | ASP | C | 281 | −5.131 | −24.917 | 5.106 | 1.00 | 45.86 | O |
| ATOM | 4350 | OD2 | ASP | C | 281 | −3.160 | −25.434 | 4.292 | 1.00 | 45.61 | O |
| ATOM | 4351 | C | ASP | C | 281 | −7.076 | −25.393 | 1.373 | 1.00 | 36.08 | C |
| ATOM | 4352 | O | ASP | C | 281 | −7.292 | −24.252 | 0.971 | 1.00 | 35.89 | O |
| ATOM | 4353 | N | ARG | C | 282 | −7.286 | −26.476 | 0.622 | 1.00 | 32.70 | N |
| ATOM | 4354 | CA | ARG | C | 282 | −7.936 | −26.364 | −0.690 | 1.00 | 31.53 | C |
| ATOM | 4355 | CB | ARG | C | 282 | −7.898 | −27.684 | −1.475 | 1.00 | 30.43 | C |
| ATOM | 4356 | CG | ARG | C | 282 | −8.676 | −27.623 | −2.790 | 1.00 | 29.05 | C |
| ATOM | 4357 | CD | ARG | C | 282 | −8.602 | −28.878 | −3.635 | 1.00 | 27.14 | C |
| ATOM | 4358 | NE | ARG | C | 282 | −9.663 | −28.866 | −4.634 | 1.00 | 25.90 | N |
| ATOM | 4359 | CZ | ARG | C | 282 | −10.166 | −29.934 | −5.259 | 1.00 | 25.67 | C |
| ATOM | 4360 | NH1 | ARG | C | 282 | −9.705 | −31.181 | −5.028 | 1.00 | 25.75 | N |
| ATOM | 4361 | NH2 | ARG | C | 282 | −11.149 | −29.759 | −6.140 | 1.00 | 25.11 | N |
| ATOM | 4362 | C | ARG | C | 282 | −9.388 | −25.898 | −0.518 | 1.00 | 31.92 | C |
| ATOM | 4363 | O | ARG | C | 282 | −9.815 | −24.944 | −1.170 | 1.00 | 30.37 | O |
| ATOM | 4364 | N | ARG | C | 283 | −10.132 | −26.573 | 0.363 | 1.00 | 33.01 | N |
| ATOM | 4365 | CA | ARG | C | 283 | −11.545 | −26.244 | 0.600 | 1.00 | 32.83 | C |
| ATOM | 4366 | CB | ARG | C | 283 | −12.185 | −27.220 | 1.591 | 1.00 | 32.27 | C |
| ATOM | 4367 | CG | ARG | C | 283 | −13.703 | −27.258 | 1.551 | 1.00 | 31.78 | C |
| ATOM | 4368 | CD | ARG | C | 283 | −14.259 | −28.192 | 2.626 | 1.00 | 31.55 | C |
| ATOM | 4369 | NE | ARG | C | 283 | −13.913 | −29.595 | 2.393 | 1.00 | 31.24 | N |
| ATOM | 4370 | CZ | ARG | C | 283 | −14.220 | −30.611 | 3.206 | 1.00 | 30.30 | C |
| ATOM | 4371 | NH1 | ARG | C | 283 | −13.841 | −31.853 | 2.889 | 1.00 | 29.10 | N |
| ATOM | 4372 | NH2 | ARG | C | 283 | −14.900 | −30.407 | 4.332 | 1.00 | 30.36 | N |
| ATOM | 4373 | C | ARG | C | 283 | −11.740 | −24.796 | 1.070 | 1.00 | 33.04 | C |
| ATOM | 4374 | O | ARG | C | 283 | −12.712 | −24.142 | 0.667 | 1.00 | 34.16 | O |
| ATOM | 4375 | N | THR | C | 284 | −10.810 | −24.297 | 1.888 | 1.00 | 33.87 | N |
| ATOM | 4376 | CA | THR | C | 284 | −10.845 | −22.915 | 2.368 | 1.00 | 34.50 | C |
| ATOM | 4377 | CB | THR | C | 284 | −9.847 | −22.698 | 3.524 | 1.00 | 35.02 | C |
| ATOM | 4378 | OG1 | THR | C | 284 | −10.090 | −23.667 | 4.552 | 1.00 | 35.39 | O |
| ATOM | 4379 | CG2 | THR | C | 284 | −9.982 | −21.284 | 4.118 | 1.00 | 35.09 | C |
| ATOM | 4380 | C | THR | C | 284 | −10.553 | −21.896 | 1.264 | 1.00 | 35.17 | C |
| ATOM | 4381 | O | THR | C | 284 | −11.287 | −20.927 | 1.115 | 1.00 | 33.58 | O |
| ATOM | 4382 | N | GLU | C | 285 | −9.477 | −22.110 | 0.504 | 1.00 | 38.14 | N |
| ATOM | 4383 | CA | GLU | C | 285 | −9.126 | −21.204 | −0.605 | 1.00 | 39.45 | C |
| ATOM | 4384 | CB | GLU | C | 285 | −7.741 | −21.522 | −1.190 | 1.00 | 39.78 | C |
| ATOM | 4385 | CG | GLU | C | 285 | −6.589 | −21.141 | −0.275 | 1.00 | 39.96 | C |
| ATOM | 4386 | CD | GLU | C | 285 | −5.216 | −21.236 | −0.940 | 1.00 | 41.08 | C |
| ATOM | 4387 | OE1 | GLU | C | 285 | −5.097 | −21.133 | −2.177 | 1.00 | 41.82 | O |
| ATOM | 4388 | OE2 | GLU | C | 285 | −4.226 | −21.388 | −0.204 | 1.00 | 41.80 | O |
| ATOM | 4389 | C | GLU | C | 285 | −10.166 | −21.197 | −1.730 | 1.00 | 41.38 | C |
| ATOM | 4390 | O | GLU | C | 285 | −10.301 | −20.182 | −2.419 | 1.00 | 42.73 | O |
| ATOM | 4391 | N | GLU | C | 286 | −10.898 | −22.302 | −1.901 | 1.00 | 43.30 | N |
| ATOM | 4392 | CA | GLU | C | 286 | −11.972 | −22.380 | −2.887 | 1.00 | 46.55 | C |
| ATOM | 4393 | CB | GLU | C | 286 | −12.265 | −23.836 | −3.266 | 1.00 | 43.92 | C |
| ATOM | 4394 | CG | GLU | C | 286 | −11.195 | −24.421 | −4.179 | 1.00 | 42.13 | C |
| ATOM | 4395 | CD | GLU | C | 286 | −11.424 | −25.863 | −4.581 | 1.00 | 41.47 | C |
| ATOM | 4396 | OE1 | GLU | C | 286 | −12.393 | −26.491 | −4.090 | 1.00 | 40.06 | O |
| ATOM | 4397 | OE2 | GLU | C | 286 | −10.608 | −26.369 | −5.377 | 1.00 | 40.77 | O |
| ATOM | 4398 | C | GLU | C | 286 | −13.259 | −21.660 | −2.462 | 1.00 | 52.99 | C |
| ATOM | 4399 | O | GLU | C | 286 | −14.029 | −21.234 | −3.358 | 1.00 | 52.81 | O |
| ATOM | 4400 | N | GLU | C | 287 | −13.492 | −21.496 | −1.158 | 1.00 | 59.55 | N |
| ATOM | 4401 | CA | GLU | C | 287 | −14.586 | −20.572 | −0.736 | 1.00 | 65.04 | C |
| ATOM | 4402 | CB | GLU | C | 287 | −15.209 | −20.909 | 0.616 | 1.00 | 63.54 | C |
| ATOM | 4403 | CG | GLU | C | 287 | −14.438 | −20.521 | 1.858 | 1.00 | 62.68 | C |
| ATOM | 4404 | CD | GLU | C | 287 | −15.100 | −21.051 | 3.138 | 1.00 | 62.64 | C |
| ATOM | 4405 | OE1 | GLU | C | 287 | −16.335 | −21.281 | 3.130 | 1.00 | 60.42 | O |
| ATOM | 4406 | OE2 | GLU | C | 287 | −14.384 | −21.247 | 4.152 | 1.00 | 61.47 | O |
| ATOM | 4407 | C | GLU | C | 287 | −14.189 | −19.094 | −0.893 | 1.00 | 72.45 | C |
| ATOM | 4408 | O | GLU | C | 287 | −14.986 | −18.243 | −1.319 | 1.00 | 73.11 | O |
| ATOM | 4409 | N | ASN | C | 288 | −12.952 | −18.793 | −0.554 | 1.00 | 80.48 | N |
| ATOM | 4410 | CA | ASN | C | 288 | −12.360 | −17.447 | −0.557 | 1.00 | 86.08 | C |
| ATOM | 4411 | CB | ASN | C | 288 | −10.921 | −17.493 | −0.014 | 1.00 | 84.34 | C |
| ATOM | 4412 | CG | ASN | C | 288 | −10.832 | −17.777 | 1.488 | 1.00 | 83.87 | C |
| ATOM | 4413 | OD1 | ASN | C | 288 | −11.823 | −17.992 | 2.192 | 1.00 | 84.55 | O |
| ATOM | 4414 | ND2 | ASN | C | 288 | −9.616 | −17.751 | 1.976 | 1.00 | 82.74 | N |
| ATOM | 4415 | C | ASN | C | 288 | −12.266 | −16.755 | −1.923 | 1.00 | 93.79 | C |
| ATOM | 4416 | O | ASN | C | 288 | −12.209 | −15.541 | −1.941 | 1.00 | 95.67 | O |
| ATOM | 4417 | N | LEU | C | 289 | −12.132 | −17.517 | −3.010 | 1.00 | 100.15 | N |
| ATOM | 4418 | CA | LEU | C | 289 | −11.892 | −16.920 | −4.338 | 1.00 | 103.41 | C |
| ATOM | 4419 | CB | LEU | C | 289 | −11.487 | −17.998 | −5.368 | 1.00 | 105.02 | C |
| ATOM | 4420 | CG | LEU | C | 289 | −10.991 | −17.539 | −6.751 | 1.00 | 104.86 | C |
| ATOM | 4421 | CD1 | LEU | C | 289 | −9.652 | −16.814 | −6.671 | 1.00 | 104.08 | C |
| ATOM | 4422 | CD2 | LEU | C | 289 | −10.896 | −18.730 | −7.693 | 1.00 | 103.96 | C |
| ATOM | 4423 | C | LEU | C | 289 | −13.099 | −16.131 | −4.848 | 1.00 | 107.00 | C |
| ATOM | 4424 | O | LEU | C | 289 | −12.929 | −15.092 | −5.485 | 1.00 | 105.92 | O |
| ATOM | 4425 | N | ARG | C | 290 | −14.295 | −16.631 | −4.544 | 1.00 | 111.17 | N |
| ATOM | 4426 | CA | ARG | C | 290 | −15.552 | −16.081 | −5.048 | 1.00 | 112.70 | C |
| ATOM | 4427 | CB | ARG | C | 290 | −16.678 | −17.108 | −4.825 | 1.00 | 114.46 | C |
| ATOM | 4428 | CG | ARG | C | 290 | −17.866 | −16.955 | −5.777 | 1.00 | 115.40 | C |
| ATOM | 4429 | CD | ARG | C | 290 | −19.175 | −17.502 | −5.211 | 1.00 | 116.55 | C |
| ATOM | 4430 | NE | ARG | C | 290 | −19.450 | −17.086 | −3.829 | 1.00 | 117.89 | N |
| ATOM | 4431 | CZ | ARG | C | 290 | −19.791 | −15.856 | −3.434 | 1.00 | 117.92 | C |
| ATOM | 4432 | NH1 | ARG | C | 290 | −20.005 | −15.622 | −2.139 | 1.00 | 118.05 | N |
| ATOM | 4433 | NH2 | ARG | C | 290 | −19.927 | −14.853 | -4.304 | 1.00 | 117.54 | N |
| ATOM | 4434 | C | ARG | C | 290 | −15.942 | −14.720 | −4.431 | 1.00 | 112.95 | C |
| ATOM | 4435 | O | ARG | C | 290 | −16.812 | −14.032 | −4.968 | 1.00 | 113.58 | O |
| ATOM | 4436 | N | LYS | C | 291 | −15.307 | −14.327 | −3.323 | 1.00 | 112.11 | N |
| ATOM | 4437 | CA | LYS | C | 291 | −15.568 | −13.025 | −2.690 | 1.00 | 110.86 | C |
| ATOM | 4438 | CB | LYS | C | 291 | −14.971 | −12.975 | −1.279 | 1.00 | 112.90 | C |
| ATOM | 4439 | CG | LYS | C | 291 | −15.581 | −13.972 | −0.306 | 1.00 | 114.57 | C |
| ATOM | 4440 | CD | LYS | C | 291 | −14.758 | −14.100 | 0.966 | 1.00 | 116.27 | C |
| ATOM | 4441 | CE | LYS | C | 291 | −15.125 | −15.361 | 1.734 | 1.00 | 117.47 | C |
| ATOM | 4442 | NZ | LYS | C | 291 | −14.339 | −15.508 | 2.990 | 1.00 | 117.53 | N |
| ATOM | 4443 | C | LYS | C | 291 | −15.006 | −11.874 | −3.523 | 1.00 | 109.15 | C |
| ATOM | 4444 | O | LYS | C | 291 | −13.790 | −11.699 | −3.623 | 1.00 | 105.73 | O |
| TER | 4445 | | LYS | C | 291 | | | | | | |
| ATOM | 4446 | N | SER | D | 96 | 16.247 | −18.570 | 4.253 | 1.00 | 66.03 | N |
| ATOM | 4447 | CA | SER | D | 96 | 17.152 | −18.575 | 3.064 | 1.00 | 66.92 | C |
| ATOM | 4448 | CB | SER | D | 96 | 16.403 | −18.128 | 1.801 | 1.00 | 67.17 | C |
| ATOM | 4449 | OG | SER | D | 96 | 17.264 | −18.110 | 0.672 | 1.00 | 66.47 | O |
| ATOM | 4450 | C | SER | D | 96 | 18.374 | −17.683 | 3.298 | 1.00 | 67.40 | C |
| ATOM | 4451 | O | SER | D | 96 | 18.264 | −16.606 | 3.886 | 1.00 | 68.71 | O |
| ATOM | 4452 | N | VAL | D | 97 | 19.523 | −18.140 | 2.805 | 1.00 | 68.09 | N |
| ATOM | 4453 | CA | VAL | D | 97 | 20.816 | −17.491 | 3.056 | 1.00 | 66.30 | C |
| ATOM | 4454 | CB | VAL | D | 97 | 21.972 | −18.501 | 2.779 | 1.00 | 66.88 | C |
| ATOM | 4455 | CG1 | VAL | D | 97 | 23.344 | −17.847 | 2.944 | 1.00 | 66.56 | C |
| ATOM | 4456 | CG2 | VAL | D | 97 | 21.846 | −19.737 | 3.667 | 1.00 | 67.45 | C |
| ATOM | 4457 | C | VAL | D | 97 | 20.963 | −16.307 | 2.104 | 1.00 | 65.71 | C |
| ATOM | 4458 | O | VAL | D | 97 | 20.983 | −16.501 | 0.883 | 1.00 | 68.11 | O |
| ATOM | 4459 | N | PRO | D | 98 | 21.142 | −15.070 | 2.619 | 1.00 | 63.91 | N |
| ATOM | 4460 | CA | PRO | D | 98 | 21.658 | −13.997 | 1.714 | 1.00 | 63.73 | C |
| ATOM | 4461 | CB | PRO | D | 98 | 21.618 | −12.725 | 2.557 | 1.00 | 63.06 | C |
| ATOM | 4462 | CG | PRO | D | 98 | 21.217 | −13.116 | 3.952 | 1.00 | 63.18 | C |
| ATOM | 4463 | CD | PRO | D | 98 | 20.826 | −14.581 | 3.973 | 1.00 | 64.21 | C |
| ATOM | 4464 | C | PRO | D | 98 | 23.076 | −14.321 | 1.275 | 1.00 | 61.37 | C |
| ATOM | 4465 | O | PRO | D | 98 | 23.850 | −14.843 | 2.070 | 1.00 | 60.29 | O |
| ATOM | 4466 | N | SER | D | 99 | 23.430 | −13.993 | 0.039 | 1.00 | 60.14 | N |
| ATOM | 4467 | CA | SER | D | 99 | 24.806 | −14.202 | −0.427 | 1.00 | 58.88 | C |
| ATOM | 4468 | CB | SER | D | 99 | 24.936 | −13.991 | −1.925 | 1.00 | 58.33 | C |
| ATOM | 4469 | OG | SER | D | 99 | 26.302 | −13.976 | −2.303 | 1.00 | 58.65 | O |
| ATOM | 4470 | C | SER | D | 99 | 25.767 | −13.279 | 0.319 | 1.00 | 58.23 | C |
| ATOM | 4471 | O | SER | D | 99 | 25.430 | −12.129 | 0.605 | 1.00 | 61.90 | O |
| ATOM | 4472 | N | GLN | D | 100 | 26.950 | −13.801 | 0.624 | 1.00 | 57.96 | N |
| ATOM | 4473 | CA | GLN | D | 100 | 28.028 | −13.015 | 1.226 | 1.00 | 57.09 | C |
| ATOM | 4474 | CB | GLN | D | 100 | 28.358 | −13.534 | 2.630 | 1.00 | 60.65 | C |
| ATOM | 4475 | CG | GLN | D | 100 | 28.973 | −14.908 | 2.704 | 1.00 | 63.38 | C |
| ATOM | 4476 | CD | GLN | D | 100 | 28.688 | −15.581 | 4.066 | 1.00 | 65.88 | C |
| ATOM | 4477 | OE1 | GLN | D | 100 | 27.561 | −16.016 | 4.302 | 1.00 | 65.32 | O |
| ATOM | 4478 | NE2 | GLN | D | 100 | 29.692 | −15.639 | 4.964 | 1.00 | 68.27 | N |
| ATOM | 4479 | C | GLN | D | 100 | 29.286 | −12.972 | 0.358 | 1.00 | 53.08 | C |
| ATOM | 4480 | O | GLN | D | 100 | 30.344 | −12.535 | 0.822 | 1.00 | 49.32 | O |
| ATOM | 4481 | N | LYS | D | 101 | 29.168 | −13.389 | −0.898 | 1.00 | 52.29 | N |
| ATOM | 4482 | CA | LYS | D | 101 | 30.313 | −13.447 | −1.821 | 1.00 | 51.09 | C |
| ATOM | 4483 | CB | LYS | D | 101 | 29.935 | −14.190 | −3.110 | 1.00 | 53.93 | C |
| ATOM | 4484 | CG | LYS | D | 101 | 31.086 | −14.379 | −4.097 | 1.00 | 56.59 | C |
| ATOM | 4485 | CD | LYS | D | 101 | 31.964 | −15.539 | −3.645 | 1.00 | 57.70 | C |
| ATOM | 4486 | CE | LYS | D | 101 | 33.220 | −15.744 | −4.511 | 1.00 | 58.99 | C |
| ATOM | 4487 | NZ | LYS | D | 101 | 32.995 | −15.945 | −5.983 | 1.00 | 59.03 | N |
| ATOM | 4488 | C | LYS | D | 101 | 30.764 | −12.025 | −2.148 | 1.00 | 46.67 | C |
| ATOM | 4489 | O | LYS | D | 101 | 29.961 | −11.195 | −2.554 | 1.00 | 45.02 | O |
| ATOM | 4490 | N | THR | D | 102 | 32.046 | −11.753 | −1.955 | 1.00 | 43.85 | N |
| ATOM | 4491 | CA | THR | D | 102 | 32.622 | −10.450 | −2.283 | 1.00 | 43.23 | C |
| ATOM | 4492 | CB | THR | D | 102 | 34.093 | −10.372 | −1.832 | 1.00 | 42.02 | C |
| ATOM | 4493 | OG1 | THR | D | 102 | 34.172 | −10.642 | −0.430 | 1.00 | 42.97 | O |
| ATOM | 4494 | CG2 | THR | D | 102 | 34.691 | −8.995 | −2.132 | 1.00 | 41.37 | C |
| ATOM | 4495 | C | THR | D | 102 | 32.529 | −10.196 | −3.794 | 1.00 | 43.03 | C |
| ATOM | 4496 | O | THR | D | 102 | 32.728 | −11.120 | −4.602 | 1.00 | 40.67 | O |
| ATOM | 4497 | N | TYR | D | 103 | 32.221 | −8.959 | −4.149 | 1.00 | 44.75 | N |
| ATOM | 4498 | CA | TYR | D | 103 | 32.055 | −8.579 | −5.537 | 1.00 | 45.45 | C |
| ATOM | 4499 | CB | TYR | D | 103 | 30.671 | −9.035 | −6.078 | 1.00 | 49.47 | C |
| ATOM | 4500 | CG | TYR | D | 103 | 30.695 | −9.288 | −7.577 | 1.00 | 54.33 | C |
| ATOM | 4501 | CD1 | TYR | D | 103 | 31.351 | −10.411 | −8.085 | 1.00 | 56.59 | C |
| ATOM | 4502 | CE1 | TYR | D | 103 | 31.397 | −10.659 | −9.453 | 1.00 | 60.26 | C |
| ATOM | 4503 | CZ | TYR | D | 103 | 30.784 | −9.773 | −10.335 | 1.00 | 59.87 | C |
| ATOM | 4504 | OH | TYR | D | 103 | 30.818 | −10.007 | −11.694 | 1.00 | 60.31 | O |
| ATOM | 4505 | CE2 | TYR | D | 103 | 30.133 | −8.650 | −9.846 | 1.00 | 60.23 | C |
| ATOM | 4506 | CD2 | TYR | D | 103 | 30.084 | −8.410 | −8.485 | 1.00 | 57.98 | C |
| ATOM | 4507 | C | TYR | D | 103 | 32.217 | −7.073 | −5.693 | 1.00 | 42.91 | C |
| ATOM | 4508 | O | TYR | D | 103 | 31.368 | −6.309 | −5.239 | 1.00 | 42.94 | O |
| ATOM | 4509 | N | GLN | D | 104 | 33.328 | −6.652 | −6.299 | 1.00 | 42.21 | N |
| ATOM | 4510 | CA | GLN | D | 104 | 33.604 | −5.231 | −6.519 | 1.00 | 41.16 | C |
| ATOM | 4511 | CB | GLN | D | 104 | 35.065 | −5.017 | −6.938 | 1.00 | 42.14 | C |
| ATOM | 4512 | CG | GLN | D | 104 | 36.083 | −5.427 | −5.880 | 1.00 | 43.26 | C |
| ATOM | 4513 | CD | GLN | D | 104 | 37.444 | −4.750 | −6.036 | 1.00 | 43.76 | C |
| ATOM | 4514 | OE1 | GLN | D | 104 | 37.735 | −4.121 | −7.053 | 1.00 | 44.94 | O |
| ATOM | 4515 | NE2 | GLN | D | 104 | 38.297 | −4.908 | −5.030 | 1.00 | 45.15 | N |
| ATOM | 4516 | C | GLN | D | 104 | 32.677 | −4.601 | −7.560 | 1.00 | 39.32 | C |
| ATOM | 4517 | O | GLN | D | 104 | 32.384 | −3.408 | −7.477 | 1.00 | 38.19 | O |
| ATOM | 4518 | N | GLY | D | 105 | 32.246 | −5.393 | −8.544 | 1.00 | 39.16 | N |
| ATOM | 4519 | CA | GLY | D | 105 | 31.268 | −4.940 | −9.531 | 1.00 | 39.53 | C |
| ATOM | 4520 | C | GLY | D | 105 | 31.818 | −3.936 | −10.532 | 1.00 | 39.11 | C |
| ATOM | 4521 | O | GLY | D | 105 | 33.023 | −3.694 | −10.581 | 1.00 | 38.75 | O |
| ATOM | 4522 | N | SER | D | 106 | 30.918 | −3.350 | −11.310 | 1.00 | 39.19 | N |
| ATOM | 4523 | CA | SER | D | 106 | 31.264 | −2.412 | −12.388 | 1.00 | 38.92 | C |
| ATOM | 4524 | CB | SER | D | 106 | 30.015 | −1.947 | −13.155 | 1.00 | 39.91 | C |
| ATOM | 4525 | OG | SER | D | 106 | 28.975 | −2.903 | −13.241 | 1.00 | 41.68 | O |
| ATOM | 4526 | C | SER | D | 106 | 31.973 | −1.158 | −11.895 | 1.00 | 37.25 | C |
| ATOM | 4527 | O | SER | D | 106 | 32.883 | −0.646 | −12.559 | 1.00 | 37.21 | O |
| ATOM | 4528 | N | TYR | D | 107 | 31.544 | −0.668 | −10.738 | 1.00 | 35.32 | N |
| ATOM | 4529 | CA | TYR | D | 107 | 32.027 | 0.596 | −10.181 | 1.00 | 34.32 | C |
| ATOM | 4530 | CB | TYR | D | 107 | 30.874 | 1.275 | −9.433 | 1.00 | 35.54 | C |
| ATOM | 4531 | CG | TYR | D | 107 | 29.701 | 1.537 | −10.344 | 1.00 | 36.44 | C |
| ATOM | 4532 | CD1 | TYR | D | 107 | 29.698 | 2.637 | −11.200 | 1.00 | 36.55 | C |
| ATOM | 4533 | CE1 | TYR | D | 107 | 28.635 | 2.878 | −12.059 | 1.00 | 37.01 | C |
| ATOM | 4534 | CZ | TYR | D | 107 | 27.563 | 2.015 | −12.076 | 1.00 | 38.12 | C |
| ATOM | 4535 | OH | TYR | D | 107 | 26.507 | 2.250 | −12.926 | 1.00 | 39.32 | O |
| ATOM | 4536 | CE2 | TYR | D | 107 | 27.542 | 0.904 | −11.245 | 1.00 | 39.06 | C |
| ATOM | 4537 | CD2 | TYR | D | 107 | 28.610 | 0.672 | −10.383 | 1.00 | 37.71 | C |
| ATOM | 4538 | C | TYR | D | 107 | 33.262 | 0.450 | −9.273 | 1.00 | 32.57 | C |
| ATOM | 4539 | O | TYR | D | 107 | 33.762 | 1.452 | −8.763 | 1.00 | 32.36 | O |
| ATOM | 4540 | N | GLY | D | 108 | 33.756 | −0.773 | −9.081 | 1.00 | 32.52 | N |
| ATOM | 4541 | CA | GLY | D | 108 | 34.944 | −1.003 | −8.251 | 1.00 | 33.12 | C |
| ATOM | 4542 | C | GLY | D | 108 | 34.699 | −0.693 | −6.785 | 1.00 | 32.72 | C |
| ATOM | 4543 | O | GLY | D | 108 | 35.456 | 0.054 | −6.163 | 1.00 | 33.12 | O |
| ATOM | 4544 | N | PHE | D | 109 | 33.629 | −1.276 | −6.251 | 1.00 | 32.64 | N |
| ATOM | 4545 | CA | PHE | D | 109 | 33.187 | −1.031 | −4.862 | 1.00 | 32.59 | C |
| ATOM | 4546 | CB | PHE | D | 109 | 31.717 | −1.446 | −4.710 | 1.00 | 32.59 | C |
| ATOM | 4547 | CG | PHE | D | 109 | 31.188 | −1.362 | −3.304 | 1.00 | 32.33 | C |
| ATOM | 4548 | CD1 | PHE | D | 109 | 31.232 | −0.159 | −2.593 | 1.00 | 32.36 | C |
| ATOM | 4549 | CE1 | PHE | D | 109 | 30.746 | −0.086 | −1.297 | 1.00 | 32.59 | C |
| ATOM | 4550 | CZ | PHE | D | 109 | 30.197 | −1.207 | −0.699 | 1.00 | 32.24 | C |
| ATOM | 4551 | CE2 | PHE | D | 109 | 30.133 | −2.407 | −1.389 | 1.00 | 33.26 | C |
| ATOM | 4552 | CD2 | PHE | D | 109 | 30.621 | −2.486 | −2.688 | 1.00 | 33.11 | C |
| ATOM | 4553 | C | PHE | D | 109 | 34.060 | −1.800 | −3.868 | 1.00 | 32.58 | C |
| ATOM | 4554 | O | PHE | D | 109 | 34.177 | −3.022 | −3.953 | 1.00 | 31.24 | O |
| ATOM | 4555 | N | ARG | D | 110 | 34.665 | −1.072 | −2.933 | 1.00 | 33.53 | N |
| ATOM | 4556 | CA | ARG | D | 110 | 35.498 | −1.664 | −1.875 | 1.00 | 35.93 | C |
| ATOM | 4557 | CB | ARG | D | 110 | 36.999 | −1.533 | −2.210 | 1.00 | 37.68 | C |
| ATOM | 4558 | CG | ARG | D | 110 | 37.389 | −1.826 | −3.651 | 1.00 | 41.53 | C |
| ATOM | 4559 | CD | ARG | D | 110 | 38.879 | −1.666 | −3.915 | 1.00 | 43.69 | C |
| ATOM | 4560 | NE | ARG | D | 110 | 39.397 | −0.332 | −3.575 | 1.00 | 44.93 | N |
| ATOM | 4561 | CZ | ARG | D | 110 | 39.139 | 0.799 | −4.240 | 1.00 | 47.00 | C |
| ATOM | 4562 | NH1 | ARG | D | 110 | 38.361 | 0.819 | −5.326 | 1.00 | 49.36 | N |
| ATOM | 4563 | NH2 | ARG | D | 110 | 39.701 | 1.939 | −3.844 | 1.00 | 48.08 | N |
| ATOM | 4564 | C | ARG | D | 110 | 35.203 | −0.914 | −0.564 | 1.00 | 34.92 | C |
| ATOM | 4565 | O | ARG | D | 110 | 34.714 | 0.232 | −0.581 | 1.00 | 34.07 | O |
| ATOM | 4566 | N | LEU | D | 111 | 35.527 | −1.567 | 0.551 | 1.00 | 34.77 | N |
| ATOM | 4567 | CA | LEU | D | 111 | 35.398 | −0.948 | 1.872 | 1.00 | 35.59 | C |
| ATOM | 4568 | CB | LEU | D | 111 | 34.793 | −1.920 | 2.883 | 1.00 | 35.71 | C |
| ATOM | 4569 | CG | LEU | D | 111 | 33.382 | −2.422 | 2.612 | 1.00 | 34.49 | C |
| ATOM | 4570 | CD1 | LEU | D | 111 | 33.014 | −3.472 | 3.647 | 1.00 | 35.04 | C |
| ATOM | 4571 | CD2 | LEU | D | 111 | 32.396 | −1.255 | 2.615 | 1.00 | 34.57 | C |
| ATOM | 4572 | C | LEU | D | 111 | 36.752 | −0.479 | 2.386 | 1.00 | 36.55 | C |
| ATOM | 4573 | O | LEU | D | 111 | 37.788 | −1.081 | 2.079 | 1.00 | 36.71 | O |
| ATOM | 4574 | N | GLY | D | 112 | 36.715 | 0.587 | 3.182 | 1.00 | 37.41 | N |
| ATOM | 4575 | CA | GLY | D | 112 | 37.877 | 1.099 | 3.891 | 1.00 | 37.67 | C |
| ATOM | 4576 | C | GLY | D | 112 | 37.528 | 1.331 | 5.350 | 1.00 | 37.09 | C |
| ATOM | 4577 | O | GLY | D | 112 | 36.352 | 1.459 | 5.713 | 1.00 | 37.88 | O |
| ATOM | 4578 | N | PHE | D | 113 | 38.560 | 1.374 | 6.181 | 1.00 | 34.58 | N |
| ATOM | 4579 | CA | PHE | D | 113 | 38.399 | 1.562 | 7.620 | 1.00 | 33.25 | C |
| ATOM | 4580 | CB | PHE | D | 113 | 38.497 | 0.218 | 8.337 | 1.00 | 32.50 | C |
| ATOM | 4581 | CG | PHE | D | 113 | 37.612 | −0.844 | 7.745 | 1.00 | 31.94 | C |
| ATOM | 4582 | CD1 | PHE | D | 113 | 36.287 | −0.951 | 8.129 | 1.00 | 31.92 | C |
| ATOM | 4583 | CE1 | PHE | D | 113 | 35.460 | −1.915 | 7.571 | 1.00 | 31.74 | C |
| ATOM | 4584 | CZ | PHE | D | 113 | 35.950 | −2.783 | 6.616 | 1.00 | 31.67 | C |
| ATOM | 4585 | CE2 | PHE | D | 113 | 37.274 | −2.670 | 6.205 | 1.00 | 31.63 | C |
| ATOM | 4586 | CD2 | PHE | D | 113 | 38.094 | −1.703 | 6.767 | 1.00 | 31.76 | C |
| ATOM | 4587 | C | PHE | D | 113 | 39.489 | 2.505 | 8.099 | 1.00 | 32.91 | C |
| ATOM | 4588 | O | PHE | D | 113 | 40.565 | 2.575 | 7.493 | 1.00 | 32.61 | O |
| ATOM | 4589 | N | LEU | D | 114 | 39.210 | 3.250 | 9.167 | 1.00 | 32.89 | N |
| ATOM | 4590 | CA | LEU | D | 114 | 40.225 | 4.094 | 9.778 | 1.00 | 33.51 | C |
| ATOM | 4591 | CB | LEU | D | 114 | 39.624 | 5.018 | 10.847 | 1.00 | 33.43 | C |
| ATOM | 4592 | CG | LEU | D | 114 | 38.681 | 6.132 | 10.335 | 1.00 | 32.77 | C |
| ATOM | 4593 | CD1 | LEU | D | 114 | 38.088 | 6.909 | 11.496 | 1.00 | 32.66 | C |
| ATOM | 4594 | CD2 | LEU | D | 114 | 39.385 | 7.059 | 9.356 | 1.00 | 32.76 | C |
| ATOM | 4595 | C | LEU | D | 114 | 41.316 | 3.225 | 10.385 | 1.00 | 34.22 | C |
| ATOM | 4596 | O | LEU | D | 114 | 41.062 | 2.099 | 10.804 | 1.00 | 34.77 | O |
| ATOM | 4597 | N | HIS | D | 115 | 42.539 | 3.753 | 10.398 | 1.00 | 34.61 | N |
| ATOM | 4598 | CA | HIS | D | 115 | 43.698 | 3.080 | 10.987 | 1.00 | 35.89 | C |
| ATOM | 4599 | CB | HIS | D | 115 | 44.925 | 3.279 | 10.093 | 1.00 | 38.54 | C |
| ATOM | 4600 | CG | HIS | D | 115 | 44.680 | 2.886 | 8.667 | 1.00 | 41.41 | C |
| ATOM | 4601 | ND1 | HIS | D | 115 | 44.602 | 3.811 | 7.646 | 1.00 | 42.47 | N |
| ATOM | 4602 | CE1 | HIS | D | 115 | 44.367 | 3.184 | 6.507 | 1.00 | 42.84 | C |
| ATOM | 4603 | NE2 | HIS | D | 115 | 44.267 | 1.890 | 6.753 | 1.00 | 43.06 | N |
| ATOM | 4604 | CD2 | HIS | D | 115 | 44.439 | 1.677 | 8.098 | 1.00 | 42.64 | C |
| ATOM | 4605 | C | HIS | D | 115 | 43.880 | 3.633 | 12.396 | 1.00 | 33.99 | C |
| ATOM | 4606 | O | HIS | D | 115 | 44.715 | 4.506 | 12.653 | 1.00 | 30.41 | O |
| ATOM | 4607 | N | SER | D | 116 | 43.077 | 3.088 | 13.309 | 1.00 | 32.63 | N |
| ATOM | 4608 | CA | SER | D | 116 | 42.857 | 3.694 | 14.621 | 1.00 | 32.08 | C |
| ATOM | 4609 | CB | SER | D | 116 | 41.400 | 3.477 | 15.033 | 1.00 | 33.45 | C |
| ATOM | 4610 | OG | SER | D | 116 | 40.528 | 3.998 | 14.036 | 1.00 | 34.36 | O |
| ATOM | 4611 | C | SER | D | 116 | 43.788 | 3.196 | 15.723 | 1.00 | 30.56 | C |
| ATOM | 4612 | O | SER | D | 116 | 43.899 | 3.836 | 16.765 | 1.00 | 29.47 | O |
| ATOM | 4613 | N | GLY | D | 117 | 44.453 | 2.063 | 15.500 | 1.00 | 29.43 | N |
| ATOM | 4614 | CA | GLY | D | 117 | 45.430 | 1.524 | 16.437 | 1.00 | 29.15 | C |
| ATOM | 4615 | C | GLY | D | 117 | 44.752 | 0.781 | 17.569 | 1.00 | 29.05 | C |
| ATOM | 4616 | O | GLY | D | 117 | 43.532 | 0.618 | 17.563 | 1.00 | 28.08 | O |
| ATOM | 4617 | N | THR | D | 118 | 45.545 | 0.332 | 18.545 | 1.00 | 29.55 | N |
| ATOM | 4618 | CA | THR | D | 118 | 45.031 | −0.513 | 19.628 | 1.00 | 30.25 | C |
| ATOM | 4619 | CB | THR | D | 118 | 45.610 | −1.944 | 19.540 | 1.00 | 30.25 | C |
| ATOM | 4620 | OG1 | THR | D | 118 | 47.040 | −1.907 | 19.604 | 1.00 | 30.22 | O |
| ATOM | 4621 | CG2 | THR | D | 118 | 45.154 | −2.616 | 18.254 | 1.00 | 30.37 | C |
| ATOM | 4622 | C | THR | D | 118 | 45.244 | 0.055 | 21.028 | 1.00 | 31.50 | C |
| ATOM | 4623 | O | THR | D | 118 | 45.264 | −0.696 | 22.003 | 1.00 | 31.73 | O |
| ATOM | 4624 | N | ALA | D | 119 | 45.353 | 1.378 | 21.138 | 1.00 | 32.66 | N |
| ATOM | 4625 | CA | ALA | D | 119 | 45.434 | 2.033 | 22.452 | 1.00 | 33.65 | C |
| ATOM | 4626 | CB | ALA | D | 119 | 45.662 | 3.531 | 22.300 | 1.00 | 33.15 | C |
| ATOM | 4627 | C | ALA | D | 119 | 44.160 | 1.763 | 23.258 | 1.00 | 35.48 | C |
| ATOM | 4628 | O | ALA | D | 119 | 43.061 | 1.676 | 22.691 | 1.00 | 34.73 | O |
| ATOM | 4629 | N | LYS | D | 120 | 44.315 | 1.640 | 24.579 | 1.00 | 38.26 | N |
| ATOM | 4630 | CA | LYS | D | 120 | 43.215 | 1.352 | 25.506 | 1.00 | 40.61 | C |
| ATOM | 4631 | CB | LYS | D | 120 | 43.722 | 1.393 | 26.961 | 1.00 | 43.99 | C |
| ATOM | 4632 | CG | LYS | D | 120 | 42.621 | 1.362 | 27.999 | 1.00 | 46.91 | C |
| ATOM | 4633 | CD | LYS | D | 120 | 43.081 | 0.847 | 29.366 | 1.00 | 49.34 | C |
| ATOM | 4634 | CE | LYS | D | 120 | 43.074 | −0.673 | 29.401 | 1.00 | 51.26 | C |
| ATOM | 4635 | NZ | LYS | D | 120 | 43.676 | −1.215 | 30.654 | 1.00 | 51.87 | N |
| ATOM | 4636 | C | LYS | D | 120 | 41.999 | 2.275 | 25.349 | 1.00 | 39.03 | C |
| ATOM | 4637 | O | LYS | D | 120 | 40.864 | 1.827 | 25.514 | 1.00 | 37.92 | O |
| ATOM | 4638 | N | SER | D | 121 | 42.242 | 3.542 | 25.008 | 1.00 | 38.43 | N |
| ATOM | 4639 | CA | SER | D | 121 | 41.174 | 4.533 | 24.821 | 1.00 | 38.89 | C |
| ATOM | 4640 | CB | SER | D | 121 | 41.761 | 5.939 | 24.944 | 1.00 | 39.36 | C |
| ATOM | 4641 | OG | SER | D | 121 | 42.613 | 6.221 | 23.848 | 1.00 | 40.79 | O |
| ATOM | 4642 | C | SER | D | 121 | 40.385 | 4.447 | 23.492 | 1.00 | 39.65 | C |
| ATOM | 4643 | O | SER | D | 121 | 39.492 | 5.260 | 23.259 | 1.00 | 39.94 | O |
| ATOM | 4644 | N | VAL | D | 122 | 40.703 | 3.482 | 22.629 | 1.00 | 39.21 | N |
| ATOM | 4645 | CA | VAL | D | 122 | 40.063 | 3.382 | 21.317 | 1.00 | 38.81 | C |
| ATOM | 4646 | CB | VAL | D | 122 | 40.975 | 2.650 | 20.292 | 1.00 | 38.35 | C |
| ATOM | 4647 | CG1 | VAL | D | 122 | 40.911 | 1.128 | 20.428 | 1.00 | 38.86 | C |
| ATOM | 4648 | CG2 | VAL | D | 122 | 40.635 | 3.094 | 18.881 | 1.00 | 38.59 | C |
| ATOM | 4649 | C | VAL | D | 122 | 38.668 | 2.751 | 21.462 | 1.00 | 38.41 | C |
| ATOM | 4650 | O | VAL | D | 122 | 38.506 | 1.711 | 22.100 | 1.00 | 37.35 | O |
| ATOM | 4651 | N | THR | D | 123 | 37.660 | 3.415 | 20.895 | 1.00 | 38.97 | N |
| ATOM | 4652 | CA | THR | D | 123 | 36.274 | 2.935 | 20.937 | 1.00 | 38.47 | C |
| ATOM | 4653 | CB | THR | D | 123 | 35.279 | 4.107 | 20.956 | 1.00 | 38.30 | C |
| ATOM | 4654 | OG1 | THR | D | 123 | 35.495 | 4.930 | 19.802 | 1.00 | 37.62 | O |
| ATOM | 4655 | CG2 | THR | D | 123 | 35.460 | 4.936 | 22.221 | 1.00 | 38.05 | C |
| ATOM | 4656 | C | THR | D | 123 | 35.938 | 2.020 | 19.765 | 1.00 | 38.02 | C |
| ATOM | 4657 | O | THR | D | 123 | 35.026 | 1.198 | 19.875 | 1.00 | 40.17 | O |
| ATOM | 4658 | N | CYS | D | 124 | 36.662 | 2.178 | 18.657 | 1.00 | 38.19 | N |
| ATOM | 4659 | CA | CYS | D | 124 | 36.455 | 1.387 | 17.447 | 1.00 | 37.31 | C |
| ATOM | 4660 | CB | CYS | D | 124 | 35.454 | 2.108 | 16.556 | 1.00 | 39.24 | C |
| ATOM | 4661 | SG | CYS | D | 124 | 35.018 | 1.229 | 15.048 | 1.00 | 44.03 | S |
| ATOM | 4662 | C | CYS | D | 124 | 37.786 | 1.195 | 16.704 | 1.00 | 34.18 | C |
| ATOM | 4663 | O | CYS | D | 124 | 38.512 | 2.169 | 16.472 | 1.00 | 32.03 | O |
| ATOM | 4664 | N | THR | D | 125 | 38.111 | −0.051 | 16.349 | 1.00 | 32.76 | N |
| ATOM | 4665 | CA | THR | D | 125 | 39.335 | −0.347 | 15.597 | 1.00 | 33.08 | C |
| ATOM | 4666 | CB | THR | D | 125 | 40.569 | −0.494 | 16.524 | 1.00 | 33.20 | C |
| ATOM | 4667 | OG1 | THR | D | 125 | 41.754 | −0.696 | 15.733 | 1.00 | 35.11 | O |
| ATOM | 4668 | CG2 | THR | D | 125 | 40.406 | −1.662 | 17.518 | 1.00 | 32.91 | C |
| ATOM | 4669 | C | THR | D | 125 | 39.187 | −1.589 | 14.719 | 1.00 | 32.26 | C |
| ATOM | 4670 | O | THR | D | 125 | 38.543 | −2.571 | 15.113 | 1.00 | 30.87 | O |
| ATOM | 4671 | N | TYR | D | 126 | 39.807 | −1.529 | 13.537 | 1.00 | 31.16 | N |
| ATOM | 4672 | CA | TYR | D | 125 | 39.711 | −2.580 | 12.524 | 1.00 | 30.08 | C |
| ATOM | 4673 | CB | TYR | D | 126 | 39.304 | −1.957 | 11.190 | 1.00 | 30.61 | C |
| ATOM | 4674 | CG | TYR | D | 126 | 39.333 | −2.910 | 10.020 | 1.00 | 30.99 | C |
| ATOM | 4675 | CD1 | TYR | D | 126 | 38.333 | −3.871 | 9.850 | 1.00 | 31.50 | C |
| ATOM | 4676 | CE1 | TYR | D | 126 | 38.358 | −4.742 | 8.772 | 1.00 | 30.88 | C |
| ATOM | 4677 | CZ | TYR | D | 126 | 39.394 | −4.662 | 7.849 | 1.00 | 30.89 | C |
| ATOM | 4678 | OH | TYR | D | 126 | 39.434 | −5.506 | 6.770 | 1.00 | 31.71 | O |
| ATOM | 4679 | CE2 | TYR | D | 126 | 40.392 | −3.720 | 7.998 | 1.00 | 31.28 | C |
| ATOM | 4680 | CD2 | TYR | D | 126 | 40.359 | −2.853 | 9.076 | 1.00 | 31.29 | C |
| ATOM | 4681 | C | TYR | D | 126 | 41.052 | −3.286 | 12.370 | 1.00 | 28.37 | C |
| ATOM | 4682 | O | TYR | D | 126 | 42.086 | −2.633 | 12.293 | 1.00 | 27.11 | O |
| ATOM | 4683 | N | SER | D | 127 | 41.007 | −4.615 | 12.317 | 1.00 | 28.59 | N |
| ATOM | 4684 | CA | SER | D | 127 | 42.180 | −5.456 | 12.097 | 1.00 | 28.60 | C |
| ATOM | 4685 | CB | SER | D | 127 | 42.098 | −6.706 | 12.971 | 1.00 | 28.54 | C |
| ATOM | 4686 | OG | SER | D | 127 | 43.023 | −7.692 | 12.564 | 1.00 | 28.12 | O |
| ATOM | 4687 | C | SER | D | 127 | 42.215 | −5.896 | 10.641 | 1.00 | 29.71 | C |
| ATOM | 4688 | O | SER | D | 127 | 41.350 | −6.671 | 10.228 | 1.00 | 31.22 | O |
| ATOM | 4689 | N | PRO | D | 128 | 43.191 | −5.397 | 9.851 | 1.00 | 29.88 | N |
| ATOM | 4690 | CA | PRO | D | 128 | 43.335 | −5.891 | 8.479 | 1.00 | 30.02 | C |
| ATOM | 4691 | CB | PRO | D | 128 | 44.507 | −5.069 | 7.929 | 1.00 | 30.42 | C |
| ATOM | 4692 | CG | PRO | D | 128 | 44.465 | −3.799 | 8.702 | 1.00 | 30.30 | C |
| ATOM | 4693 | CD | PRO | D | 128 | 43.988 | −4.174 | 10.073 | 1.00 | 29.91 | C |
| ATOM | 4694 | C | PRO | D | 128 | 43.642 | −7.387 | 8.379 | 1.00 | 30.48 | C |
| ATOM | 4695 | O | PRO | D | 128 | 43.125 | −8.051 | 7.482 | 1.00 | 29.29 | O |
| ATOM | 4696 | N | ALA | D | 129 | 44.455 | −7.910 | 9.292 | 1.00 | 30.68 | N |
| ATOM | 4697 | CA | ALA | D | 129 | 44.852 | −9.319 | 9.242 | 1.00 | 31.70 | C |
| ATOM | 4698 | CB | ALA | D | 129 | 45.927 | −9.604 | 10.282 | 1.00 | 31.69 | C |
| ATOM | 4699 | C | ALA | D | 129 | 43.667 | −10.263 | 9.427 | 1.00 | 32.37 | C |
| ATOM | 4700 | O | ALA | D | 129 | 43.599 | −11.302 | 8.784 | 1.00 | 33.91 | O |
| ATOM | 4701 | N | LEU | D | 130 | 42.741 | −9.887 | 10.304 | 1.00 | 34.43 | N |
| ATOM | 4702 | CA | LEU | D | 130 | 41.545 | −10.679 | 10.586 | 1.00 | 34.86 | C |
| ATOM | 4703 | CB | LEU | D | 130 | 41.248 | −10.621 | 12.097 | 1.00 | 36.14 | C |
| ATOM | 4704 | CG | LEU | D | 130 | 42.251 | −11.336 | 13.011 | 1.00 | 37.78 | C |
| ATOM | 4705 | CD1 | LEU | D | 130 | 42.215 | −10.829 | 14.437 | 1.00 | 38.53 | C |
| ATOM | 4706 | CD2 | LEU | D | 130 | 41.971 | −12.840 | 12.971 | 1.00 | 38.09 | C |
| ATOM | 4707 | C | LEU | D | 130 | 40.294 | −10.250 | 9.804 | 1.00 | 34.11 | C |
| ATOM | 4708 | O | LEU | D | 130 | 39.286 | −10.955 | 9.849 | 1.00 | 35.14 | O |
| ATOM | 4709 | N | ASN | D | 131 | 40.354 | −9.112 | 9.108 | 1.00 | 33.79 | N |
| ATOM | 4710 | CA | ASN | D | 131 | 39.166 | −8.480 | 8.493 | 1.00 | 32.31 | C |
| ATOM | 4711 | CB | ASN | D | 131 | 38.730 | −9.220 | 7.212 | 1.00 | 31.62 | C |
| ATOM | 4712 | CG | ASN | D | 131 | 37.639 | −8.479 | 6.439 | 1.00 | 31.39 | C |
| ATOM | 4713 | OD1 | ASN | D | 131 | 37.545 | −7.246 | 6.475 | 1.00 | 30.42 | O |
| ATOM | 4714 | ND2 | ASN | D | 131 | 36.794 | −9.238 | 5.747 | 1.00 | 30.09 | N |
| ATOM | 4715 | C | ASN | D | 131 | 38.028 | −8.402 | 9.522 | 1.00 | 31.76 | C |
| ATOM | 4716 | O | ASN | D | 131 | 36.890 | −8.850 | 9.282 | 1.00 | 30.57 | O |
| ATOM | 4717 | N | LYS | D | 132 | 38.370 | −7.847 | 10.680 | 1.00 | 31.88 | N |
| ATOM | 4718 | CA | LYS | D | 132 | 37.493 | −7.867 | 11.843 | 1.00 | 32.63 | C |
| ATOM | 4719 | CB | LYS | D | 132 | 37.965 | −8.928 | 12.828 | 1.00 | 32.84 | C |
| ATOM | 4720 | CG | LYS | D | 132 | 37.002 | −9.192 | 13.972 | 1.00 | 32.65 | C |
| ATOM | 4721 | CD | LYS | D | 132 | 37.255 | −10.580 | 14.575 | 1.00 | 32.80 | C |
| ATOM | 4722 | CE | LYS | D | 132 | 36.584 | −10.716 | 15.939 | 1.00 | 33.84 | C |
| ATOM | 4723 | NZ | LYS | D | 132 | 36.885 | −12.043 | 16.533 | 1.00 | 33.42 | N |
| ATOM | 4724 | C | LYS | D | 132 | 37.462 | −6.501 | 12.521 | 1.00 | 32.98 | C |
| ATOM | 4725 | O | LYS | D | 132 | 38.501 | −5.930 | 12.853 | 1.00 | 31.62 | O |
| ATOM | 4726 | N | MET | D | 133 | 36.250 | −5.993 | 12.718 | 1.00 | 33.47 | N |
| ATOM | 4727 | CA | MET | D | 133 | 36.034 | −4.744 | 13.426 | 1.00 | 33.80 | C |
| ATOM | 4728 | CB | MET | D | 133 | 34.829 | −4.036 | 12.823 | 1.00 | 35.09 | C |
| ATOM | 4729 | CG | MET | D | 133 | 34.504 | −2.707 | 13.457 | 1.00 | 37.77 | C |
| ATOM | 4730 | SD | MET | D | 133 | 35.683 | −1.412 | 13.060 | 1.00 | 40.65 | S |
| ATOM | 4731 | CE | MET | D | 133 | 35.175 | −1.029 | 11.386 | 1.00 | 39.52 | C |
| ATOM | 4732 | C | MET | D | 133 | 35.806 | −5.056 | 14.912 | 1.00 | 34.77 | C |
| ATOM | 4733 | O | MET | D | 133 | 35.057 | −5.981 | 15.247 | 1.00 | 31.84 | O |
| ATOM | 4734 | N | PHE | D | 134 | 36.468 | −4.299 | 15.781 | 1.00 | 35.51 | N |
| ATOM | 4735 | CA | PHE | D | 134 | 36.261 | −4.380 | 17.226 | 1.00 | 37.12 | C |
| ATOM | 4736 | CB | PHE | D | 134 | 37.584 | −4.641 | 17.950 | 1.00 | 36.46 | C |
| ATOM | 4737 | CG | PHE | D | 134 | 38.214 | −5.958 | 17.600 | 1.00 | 35.70 | C |
| ATOM | 4738 | CD1 | PHE | D | 134 | 38.934 | −6.101 | 16.423 | 1.00 | 35.69 | C |
| ATOM | 4739 | CE1 | PHE | D | 134 | 39.531 | −7.314 | 16.094 | 1.00 | 35.84 | C |
| ATOM | 4740 | CZ | PHE | D | 134 | 39.419 | −8.395 | 16.951 | 1.00 | 36.04 | C |
| ATOM | 4741 | CE2 | PHE | D | 134 | 38.711 | −8.269 | 18.145 | 1.00 | 35.82 | C |
| ATOM | 4742 | CD2 | PHE | D | 134 | 38.112 | −7.051 | 18.465 | 1.00 | 35.16 | C |
| ATOM | 4743 | C | PHE | D | 134 | 35.688 | −3.040 | 17.672 | 1.00 | 39.26 | C |
| ATOM | 4744 | O | PHE | D | 134 | 36.308 | −2.000 | 17.436 | 1.00 | 39.39 | O |
| ATOM | 4745 | N | CYS | D | 135 | 34.507 | −3.064 | 18.293 | 1.00 | 41.80 | N |
| ATOM | 4746 | CA | CYS | D | 135 | 33.843 | −1.835 | 18.742 | 1.00 | 44.84 | C |
| ATOM | 4747 | CB | CYS | D | 135 | 32.728 | −1.413 | 17.769 | 1.00 | 47.06 | C |
| ATOM | 4748 | SG | CYS | D | 135 | 32.672 | −2.049 | 16.074 | 1.00 | 53.23 | S |
| ATOM | 4749 | C | CYS | D | 135 | 33.208 | −1.994 | 20.120 | 1.00 | 44.48 | C |
| ATOM | 4750 | O | CYS | D | 135 | 32.888 | −3.091 | 20.561 | 1.00 | 41.88 | O |
| ATOM | 4751 | N | GLN | D | 136 | 33.020 | −0.857 | 20.777 | 1.00 | 45.98 | N |
| ATOM | 4752 | CA | GLN | D | 136 | 32.237 | −0.780 | 22.005 | 1.00 | 48.68 | C |
| ATOM | 4753 | CB | GLN | D | 136 | 32.711 | 0.393 | 22.875 | 1.00 | 47.82 | C |
| ATOM | 4754 | CG | GLN | D | 136 | 33.999 | 0.096 | 23.624 | 1.00 | 48.71 | C |
| ATOM | 4755 | CD | GLN | D | 136 | 34.509 | 1.253 | 24.456 | 1.00 | 48.87 | C |
| ATOM | 4756 | OE1 | GLN | D | 136 | 34.157 | 2.423 | 24.229 | 1.00 | 48.76 | O |
| ATOM | 4757 | NE2 | GLN | D | 136 | 35.358 | 0.933 | 25.419 | 1.00 | 46.49 | N |
| ATOM | 4758 | C | GLN | D | 136 | 30.754 | −0.649 | 21.646 | 1.00 | 48.61 | C |
| ATOM | 4759 | O | GLN | D | 136 | 30.384 | −0.132 | 20.592 | 1.00 | 45.84 | O |
| ATOM | 4760 | N | LEU | D | 137 | 29.914 | −1.146 | 22.550 | 1.00 | 50.53 | N |
| ATOM | 4761 | CA | LEU | D | 137 | 28.451 | −1.175 | 22.365 | 1.00 | 51.01 | C |
| ATOM | 4762 | CB | LEU | D | 137 | 27.813 | −1.854 | 23.573 | 1.00 | 51.34 | C |
| ATOM | 4763 | CG | LEU | D | 137 | 26.296 | −1.949 | 23.659 | 1.00 | 53.02 | C |
| ATOM | 4764 | CD1 | LEU | D | 137 | 26.011 | −3.282 | 24.345 | 1.00 | 53.30 | C |
| ATOM | 4765 | CD2 | LEU | D | 137 | 25.596 | −0.867 | 24.463 | 1.00 | 53.31 | C |
| ATOM | 4766 | C | LEU | D | 137 | 27.891 | 0.234 | 22.171 | 1.00 | 50.00 | C |
| ATOM | 4767 | O | LEU | D | 137 | 28.225 | 1.163 | 22.936 | 1.00 | 49.55 | O |
| ATOM | 4768 | N | ALA | D | 138 | 27.086 | 0.400 | 21.129 | 1.00 | 49.46 | N |
| ATOM | 4769 | CA | ALA | D | 138 | 26.433 | 1.680 | 20.788 | 1.00 | 49.71 | C |
| ATOM | 4770 | CB | ALA | D | 138 | 25.426 | 2.045 | 21.863 | 1.00 | 49.39 | C |
| ATOM | 4771 | C | ALA | D | 138 | 27.366 | 2.861 | 20.478 | 1.00 | 49.54 | C |
| ATOM | 4772 | O | ALA | D | 138 | 26.933 | 4.013 | 20.570 | 1.00 | 52.10 | O |
| ATOM | 4773 | N | LYS | D | 139 | 28.615 | 2.587 | 20.097 | 1.00 | 49.24 | N |
| ATOM | 4774 | CA | LYS | D | 139 | 29.552 | 3.652 | 19.731 | 1.00 | 46.96 | C |
| ATOM | 4775 | CB | LYS | D | 139 | 30.932 | 3.397 | 20.332 | 1.00 | 48.09 | C |
| ATOM | 4776 | CG | LYS | D | 139 | 30.973 | 3.365 | 21.850 | 1.00 | 49.37 | C |
| ATOM | 4777 | CD | LYS | D | 139 | 30.822 | 4.741 | 22.480 | 1.00 | 50.40 | C |
| ATOM | 4778 | CE | LYS | D | 139 | 30.792 | 4.613 | 23.996 | 1.00 | 51.89 | C |
| ATOM | 4779 | NZ | LYS | D | 139 | 31.297 | 5.808 | 24.745 | 1.00 | 51.52 | N |
| ATOM | 4780 | C | LYS | D | 139 | 29.664 | 3.780 | 18.219 | 1.00 | 44.26 | C |
| ATOM | 4781 | O | LYS | D | 139 | 29.420 | 2.820 | 17.488 | 1.00 | 41.11 | O |
| ATOM | 4782 | N | THR | D | 140 | 30.050 | 4.974 | 17.777 | 1.00 | 42.84 | N |
| ATOM | 4783 | CA | THR | D | 140 | 30.316 | 5.259 | 16.369 | 1.00 | 42.46 | C |
| ATOM | 4784 | CB | THR | D | 140 | 30.883 | 6.679 | 16.189 | 1.00 | 41.64 | C |
| ATOM | 4785 | OG1 | THR | D | 140 | 30.043 | 7.615 | 16.869 | 1.00 | 41.35 | O |
| ATOM | 4786 | CG2 | THR | D | 140 | 30.976 | 7.051 | 14.702 | 1.00 | 42.17 | C |
| ATOM | 4787 | C | THR | D | 140 | 31.313 | 4.265 | 15.772 | 1.00 | 41.92 | C |
| ATOM | 4788 | O | THR | D | 140 | 32.362 | 4.015 | 16.350 | 1.00 | 40.06 | O |
| ATOM | 4789 | N | CYS | D | 141 | 30.953 | 3.703 | 14.625 | 1.00 | 42.52 | N |
| ATOM | 4790 | CA | CYS | D | 141 | 31.774 | 2.736 | 13.922 | 1.00 | 43.43 | C |
| ATOM | 4791 | CB | CYS | D | 141 | 31.183 | 1.336 | 14.093 | 1.00 | 45.27 | C |
| ATOM | 4792 | SG | CYS | D | 141 | 32.275 | −0.004 | 13.579 | 1.00 | 49.59 | S |
| ATOM | 4793 | C | CYS | D | 141 | 31.825 | 3.177 | 12.453 | 1.00 | 42.94 | C |
| ATOM | 4794 | O | CYS | D | 141 | 30.902 | 2.876 | 11.686 | 1.00 | 43.17 | O |
| ATOM | 4795 | N | PRO | D | 142 | 32.876 | 3.932 | 12.068 | 1.00 | 41.01 | N |
| ATOM | 4796 | CA | PRO | D | 142 | 32.923 | 4.417 | 10.685 | 1.00 | 39.10 | C |
| ATOM | 4797 | CB | PRO | D | 142 | 34.020 | 5.496 | 10.700 | 1.00 | 39.46 | C |
| ATOM | 4798 | CG | PRO | D | 142 | 34.555 | 5.560 | 12.110 | 1.00 | 39.61 | C |
| ATOM | 4799 | CD | PRO | D | 142 | 34.052 | 4.358 | 12.852 | 1.00 | 39.95 | C |
| ATOM | 4800 | C | PRO | D | 142 | 33.275 | 3.306 | 9.698 | 1.00 | 37.28 | C |
| ATOM | 4801 | O | PRO | D | 142 | 34.186 | 2.512 | 9.960 | 1.00 | 36.98 | O |
| ATOM | 4802 | N | VAL | D | 143 | 32.531 | 3.242 | 8.595 | 1.00 | 34.24 | N |
| ATOM | 4803 | CA | VAL | D | 143 | 32.856 | 2.366 | 7.471 | 1.00 | 31.96 | C |
| ATOM | 4804 | CB | VAL | D | 143 | 31.831 | 1.220 | 7.308 | 1.00 | 32.00 | C |
| ATOM | 4805 | CG1 | VAL | D | 143 | 32.133 | 0.403 | 6.056 | 1.00 | 31.25 | C |
| ATOM | 4806 | CG2 | VAL | D | 143 | 31.851 | 0.320 | 8.538 | 1.00 | 32.26 | C |
| ATOM | 4807 | C | VAL | D | 143 | 32.914 | 3.237 | 6.221 | 1.00 | 30.59 | C |
| ATOM | 4808 | O | VAL | D | 143 | 31.998 | 4.019 | 5.968 | 1.00 | 29.78 | O |
| ATOM | 4809 | N | GLN | D | 144 | 33.998 | 3.100 | 5.458 | 1.00 | 30.46 | N |
| ATOM | 4810 | CA | GLN | D | 144 | 34.239 | 3.909 | 4.266 | 1.00 | 30.06 | C |
| ATOM | 4811 | CB | GLN | D | 144 | 35.718 | 4.293 | 4.163 | 1.00 | 29.99 | C |
| ATOM | 4812 | CG | GLN | D | 144 | 36.263 | 5.053 | 5.363 | 1.00 | 29.72 | C |
| ATOM | 4813 | CD | GLN | D | 144 | 37.766 | 5.277 | 5.296 | 1.00 | 29.72 | C |
| ATOM | 4814 | OE1 | GLN | D | 144 | 38.489 | 4.597 | 4.560 | 1.00 | 29.88 | O |
| ATOM | 4815 | NE2 | GLN | D | 144 | 38.245 | 6.242 | 6.072 | 1.00 | 29.94 | N |
| ATOM | 4816 | C | GLN | D | 144 | 33.850 | 3.141 | 3.010 | 1.00 | 30.21 | C |
| ATOM | 4817 | O | GLN | D | 144 | 34.096 | 1.935 | 2.913 | 1.00 | 27.93 | O |
| ATOM | 4818 | N | LEU | D | 145 | 33.248 | 3.854 | 2.060 | 1.00 | 32.26 | N |
| ATOM | 4819 | CA | LEU | D | 145 | 32.914 | 3.321 | 0.752 | 1.00 | 33.52 | C |
| ATOM | 4820 | CB | LEU | D | 145 | 31.472 | 3.679 | 0.388 | 1.00 | 32.99 | C |
| ATOM | 4821 | CG | LEU | D | 145 | 30.435 | 3.492 | 1.511 | 1.00 | 33.23 | C |
| ATOM | 4822 | CD1 | LEU | D | 145 | 29.087 | 3.940 | 1.032 | 1.00 | 33.06 | C |
| ATOM | 4823 | CD2 | LEU | D | 145 | 30.401 | 2.065 | 2.035 | 1.00 | 32.77 | C |
| ATOM | 4824 | C | LEU | D | 145 | 33.882 | 3.915 | −0.266 | 1.00 | 35.15 | C |
| ATOM | 4825 | O | LEU | D | 145 | 33.965 | 5.149 | −0.421 | 1.00 | 36.05 | O |
| ATOM | 4826 | N | TRP | D | 146 | 34.634 | 3.032 | −0.929 | 1.00 | 37.52 | N |
| ATOM | 4827 | CA | TRP | D | 146 | 35.577 | 3.427 | −1.963 | 1.00 | 39.79 | C |
| ATOM | 4828 | CB | TRP | D | 146 | 36.968 | 2.870 | −1.650 | 1.00 | 42.57 | C |
| ATOM | 4829 | CG | TRP | D | 146 | 37.647 | 3.503 | −0.451 | 1.00 | 45.00 | C |
| ATOM | 4830 | CD1 | TRP | D | 146 | 37.356 | 3.273 | 0.865 | 1.00 | 47.07 | C |
| ATOM | 4831 | NE1 | TRP | D | 146 | 38.177 | 4.014 | 1.672 | 1.00 | 48.05 | N |
| ATOM | 4832 | CE2 | TRP | D | 146 | 39.039 | 4.733 | 0.889 | 1.00 | 48.18 | C |
| ATOM | 4833 | CD2 | TRP | D | 146 | 38.730 | 4.440 | −0.461 | 1.00 | 47.16 | C |
| ATOM | 4834 | CE3 | TRP | D | 146 | 39.456 | 5.077 | −1.478 | 1.00 | 46.89 | C |
| ATOM | 4835 | CZ3 | TRP | D | 146 | 40.504 | 5.946 | −1.109 | 1.00 | 47.04 | C |
| ATOM | 4836 | CH2 | TRP | D | 146 | 40.782 | 6.213 | 0.245 | 1.00 | 47.06 | C |
| ATOM | 4837 | CZ2 | TRP | D | 146 | 40.067 | 5.623 | 1.253 | 1.00 | 48.31 | C |
| ATOM | 4838 | C | TRP | D | 146 | 35.079 | 2.864 | −3.293 | 1.00 | 39.06 | C |
| ATOM | 4839 | O | TRP | D | 146 | 34.769 | 1.667 | −3.389 | 1.00 | 37.34 | O |
| ATOM | 4840 | N | VAL | D | 147 | 35.004 | 3.723 | −4.313 | 1.00 | 38.96 | N |
| ATOM | 4841 | CA | VAL | D | 147 | 34.645 | 3.315 | −5.684 | 1.00 | 39.13 | C |
| ATOM | 4842 | CB | VAL | D | 147 | 33.211 | 3.746 | −6.078 | 1.00 | 37.58 | C |
| ATOM | 4843 | CG1 | VAL | D | 147 | 32.188 | 2.953 | −5.283 | 1.00 | 38.30 | C |
| ATOM | 4844 | CG2 | VAL | D | 147 | 32.991 | 5.249 | −5.890 | 1.00 | 37.65 | C |
| ATOM | 4845 | C | VAL | D | 147 | 35.641 | 3.862 | −6.712 | 1.00 | 39.45 | C |
| ATOM | 4846 | O | VAL | D | 147 | 36.191 | 4.950 | −6.533 | 1.00 | 39.09 | O |
| ATOM | 4847 | N | ASP | D | 148 | 35.863 | 3.094 | −7.777 | 1.00 | 40.32 | N |
| ATOM | 4848 | CA | ASP | D | 148 | 36.640 | 3.558 | −8.936 | 1.00 | 41.31 | C |
| ATOM | 4849 | CB | ASP | D | 148 | 37.111 | 2.377 | −9.802 | 1.00 | 41.50 | C |
| ATOM | 4850 | CG | ASP | D | 148 | 38.057 | 1.435 | −9.065 | 1.00 | 41.58 | C |
| ATOM | 4851 | OD1 | ASP | D | 148 | 38.743 | 1.873 | −8.117 | 1.00 | 39.87 | O |
| ATOM | 4852 | OD2 | ASP | D | 148 | 36.120 | 0.246 | −9.443 | 1.00 | 41.66 | O |
| ATOM | 4853 | C | ASP | D | 148 | 35.824 | 4.512 | −9.806 | 1.00 | 40.84 | C |
| ATOM | 4854 | O | ASP | D | 148 | 36.374 | 5.466 | −10.350 | 1.00 | 41.51 | O |
| ATOM | 4855 | N | SER | D | 149 | 34.520 | 4.247 | −9.934 | 1.00 | 40.34 | N |
| ATOM | 4856 | CA | SER | D | 149 | 33.617 | 5.066 | −10.751 | 1.00 | 39.22 | C |
| ATOM | 4857 | CB | SER | D | 149 | 33.213 | 4.308 | −12.023 | 1.00 | 39.17 | C |
| ATOM | 4858 | OG | SER | D | 149 | 34.323 | 3.652 | −12.620 | 1.00 | 39.79 | O |
| ATOM | 4859 | C | SER | D | 149 | 32.370 | 5.416 | −9.947 | 1.00 | 39.02 | C |
| ATOM | 4860 | O | SER | D | 149 | 31.762 | 4.529 | −9.342 | 1.00 | 39.24 | O |
| ATOM | 4861 | N | THR | D | 150 | 31.986 | 6.691 | −9.953 | 1.00 | 38.55 | N |
| ATOM | 4862 | CA | THR | D | 150 | 30.802 | 7.152 | −9.243 | 1.00 | 39.93 | C |
| ATOM | 4863 | CB | THR | D | 150 | 30.648 | 8.685 | −9.365 | 1.00 | 40.55 | C |
| ATOM | 4864 | OG1 | THR | D | 150 | 31.854 | 9.321 | −8.933 | 1.00 | 41.03 | O |
| ATOM | 4865 | CG2 | THR | D | 150 | 29.463 | 9.198 | −8.530 | 1.00 | 40.42 | C |
| ATOM | 4866 | C | THR | D | 150 | 29.540 | 6.467 | −9.802 | 1.00 | 42.30 | C |
| ATOM | 4867 | O | THR | D | 150 | 29.280 | 6.553 | −11.006 | 1.00 | 42.53 | O |
| ATOM | 4868 | N | PRO | D | 151 | 28.772 | 5.765 | −8.937 | 1.00 | 42.13 | N |
| ATOM | 4869 | CA | PRO | D | 151 | 27.521 | 5.167 | −9.431 | 1.00 | 41.61 | C |
| ATOM | 4870 | CB | PRO | D | 151 | 27.162 | 4.124 | −8.364 | 1.00 | 41.70 | C |
| ATOM | 4871 | CG | PRO | D | 151 | 28.000 | 4.428 | −7.161 | 1.00 | 41.92 | C |
| ATOM | 4872 | CD | PRO | D | 151 | 29.051 | 5.450 | −7.514 | 1.00 | 41.64 | C |
| ATOM | 4873 | C | PRO | D | 151 | 26.414 | 6.219 | −9.611 | 1.00 | 40.20 | C |
| ATOM | 4874 | O | PRO | D | 151 | 26.524 | 7.324 | −9.070 | 1.00 | 42.05 | O |
| ATOM | 4875 | N | PRO | D | 152 | 25.353 | 5.878 | −10.366 | 1.00 | 40.22 | N |
| ATOM | 4876 | CA | PRO | D | 152 | 24.328 | 6.867 | −10.711 | 1.00 | 40.73 | C |
| ATOM | 4877 | CB | PRO | D | 152 | 23.402 | 6.125 | −11.690 | 1.00 | 40.78 | C |
| ATOM | 4878 | CG | PRO | D | 152 | 23.749 | 4.698 | −11.621 | 1.00 | 41.11 | C |
| ATOM | 4879 | CD | PRO | D | 152 | 25.070 | 4.545 | −10.930 | 1.00 | 41.42 | C |
| ATOM | 4880 | C | PRO | D | 152 | 23.523 | 7.373 | −9.504 | 1.00 | 40.63 | C |
| ATOM | 4881 | O | PRO | D | 152 | 23.480 | 6.680 | −8.476 | 1.00 | 40.55 | O |
| ATOM | 4882 | N | PRO | D | 153 | 22.877 | 8.556 | −9.631 | 1.00 | 40.19 | N |
| ATOM | 4883 | CA | PRO | D | 153 | 21.992 | 9.053 | −8.574 | 1.00 | 38.24 | C |
| ATOM | 4884 | CB | PRO | D | 153 | 21.351 | 10.305 | −9.198 | 1.00 | 39.24 | C |
| ATOM | 4885 | CG | PRO | D | 153 | 22.294 | 10.740 | −10.268 | 1.00 | 39.70 | C |
| ATOM | 4886 | CD | PRO | D | 153 | 22.976 | 9.509 | −10.758 | 1.00 | 40.03 | C |
| ATOM | 4887 | C | PRO | D | 153 | 20.909 | 8.045 | −8.182 | 1.00 | 35.84 | C |
| ATOM | 4888 | O | PRO | D | 153 | 20.482 | 7.247 | −9.027 | 1.00 | 37.69 | O |
| ATOM | 4889 | N | GLY | D | 154 | 20.504 | 8.072 | −6.909 | 1.00 | 33.63 | N |
| ATOM | 4890 | CA | GLY | D | 154 | 19.544 | 7.125 | −6.363 | 1.00 | 31.39 | C |
| ATOM | 4891 | C | GLY | D | 154 | 20.143 | 5.805 | −5.901 | 1.00 | 30.26 | C |
| ATOM | 4892 | O | GLY | D | 154 | 19.939 | 4.890 | −5.505 | 1.00 | 29.43 | O |
| ATOM | 4893 | N | THR | D | 155 | 21.466 | 5.663 | −6.012 | 1.00 | 29.76 | N |
| ATOM | 4894 | CA | THR | D | 155 | 22.185 | 4.504 | −5.483 | 1.00 | 29.46 | C |
| ATOM | 4895 | CB | THR | D | 155 | 23.683 | 4.569 | −5.876 | 1.00 | 30.09 | C |
| ATOM | 4896 | OG1 | THR | D | 155 | 23.801 | 4.449 | −7.295 | 1.00 | 30.24 | O |
| ATOM | 4897 | CG2 | THR | D | 155 | 24.513 | 3.441 | −5.236 | 1.00 | 30.29 | C |
| ATOM | 4898 | C | THR | D | 155 | 22.033 | 4.453 | −3.963 | 1.00 | 28.31 | C |
| ATOM | 4899 | O | THR | D | 155 | 21.984 | 5.489 | −3.311 | 1.00 | 27.64 | O |
| ATOM | 4900 | N | ARG | D | 156 | 21.966 | 3.246 | −3.417 | 1.00 | 27.71 | N |
| ATOM | 4901 | CA | ARG | D | 156 | 21.753 | 3.019 | −1.978 | 1.00 | 26.44 | C |
| ATOM | 4902 | CB | ARG | D | 156 | 20.328 | 2.504 | −1.734 | 1.00 | 25.61 | C |
| ATOM | 4903 | CG | ARG | D | 156 | 19.311 | 3.614 | −1.504 | 1.00 | 25.66 | C |
| ATOM | 4904 | CD | ARG | D | 156 | 17.958 | 3.349 | −2.166 | 1.00 | 25.56 | C |
| ATOM | 4905 | NE | ARG | D | 156 | 17.230 | 2.206 | −1.600 | 1.00 | 25.51 | N |
| ATOM | 4906 | CZ | ARG | D | 156 | 16.351 | 2.245 | −0.591 | 1.00 | 25.21 | C |
| ATOM | 4907 | NH1 | ARG | D | 156 | 16.071 | 3.382 | 0.056 | 1.00 | 24.52 | N |
| ATOM | 4908 | NH2 | ARG | D | 156 | 15.752 | 1.116 | −0.212 | 1.00 | 24.50 | N |
| ATOM | 4909 | C | ARG | D | 156 | 22.780 | 2.056 | −1.391 | 1.00 | 26.02 | C |
| ATOM | 4910 | O | ARG | D | 156 | 23.351 | 1.231 | −2.114 | 1.00 | 23.98 | O |
| ATOM | 4911 | N | VAL | D | 157 | 22.993 | 2.185 | −0.074 | 1.00 | 26.17 | N |
| ATOM | 4912 | CA | VAL | D | 157 | 23.975 | 1.396 | 0.667 | 1.00 | 25.90 | C |
| ATOM | 4913 | CB | VAL | D | 157 | 25.096 | 2.292 | 1.230 | 1.00 | 26.80 | C |
| ATOM | 4914 | CG1 | VAL | D | 157 | 26.261 | 1.448 | 1.737 | 1.00 | 27.35 | C |
| ATOM | 4915 | CG2 | VAL | D | 157 | 25.583 | 3.273 | 0.177 | 1.00 | 27.28 | C |
| ATOM | 4916 | C | VAL | D | 157 | 23.273 | 0.680 | 1.820 | 1.00 | 25.34 | C |
| ATOM | 4917 | O | VAL | D | 157 | 22.759 | 1.331 | 2.737 | 1.00 | 25.00 | O |
| ATOM | 4918 | N | ARG | D | 158 | 23.268 | −0.655 | 1.777 | 1.00 | 25.05 | N |
| ATOM | 4919 | CA | ARG | D | 158 | 22.604 | −1.476 | 2.783 | 1.00 | 24.96 | C |
| ATOM | 4920 | CB | ARG | D | 158 | 21.682 | −2.486 | 2.097 | 1.00 | 25.14 | C |
| ATOM | 4921 | CG | ARG | D | 158 | 20.800 | −3.309 | 3.021 | 1.00 | 25.18 | C |
| ATOM | 4922 | CD | ARG | D | 158 | 19.925 | −4.272 | 2.222 | 1.00 | 25.27 | C |
| ATOM | 4923 | NE | ARG | D | 158 | 19.069 | −3.546 | 1.275 | 1.00 | 25.47 | N |
| ATOM | 4924 | CZ | ARG | D | 158 | 17.898 | −2.982 | 1.577 | 1.00 | 25.79 | C |
| ATOM | 4925 | NH1 | ARG | D | 158 | 17.358 | −3.075 | 2.795 | 1.00 | 26.45 | N |
| ATOM | 4926 | NH2 | ARG | D | 158 | 17.237 | −2.326 | 0.632 | 1.00 | 25.90 | N |
| ATOM | 4927 | C | ARG | D | 158 | 23.616 | −2.215 | 3.636 | 1.00 | 24.52 | C |
| ATOM | 4928 | O | ARG | D | 158 | 24.606 | −2.700 | 3.118 | 1.00 | 24.86 | O |
| ATOM | 4929 | N | ALA | D | 159 | 23.343 | −2.315 | 4.936 | 1.00 | 24.69 | N |
| ATOM | 4930 | CA | ALA | D | 159 | 24.112 | −3.152 | 5.853 | 1.00 | 24.83 | C |
| ATOM | 4931 | CB | ALA | D | 159 | 24.749 | −2.316 | 6.946 | 1.00 | 24.89 | C |
| ATOM | 4932 | C | ALA | D | 159 | 23.190 | −4.210 | 6.455 | 1.00 | 25.32 | C |
| ATOM | 4933 | O | ALA | D | 159 | 22.037 | −3.928 | 6.741 | 1.00 | 24.76 | O |
| ATOM | 4934 | N | MET | D | 160 | 23.708 | −5.426 | 6.637 | 1.00 | 26.32 | N |
| ATOM | 4935 | CA | MET | D | 160 | 22.955 | −6.555 | 7.200 | 1.00 | 27.98 | C |
| ATOM | 4936 | CB | MET | D | 160 | 22.275 | −7.349 | 6.066 | 1.00 | 28.63 | C |
| ATOM | 4937 | CG | MET | D | 160 | 21.602 | −8.664 | 6.484 | 1.00 | 29.76 | C |
| ATOM | 4938 | SD | MET | D | 160 | 21.042 | −9.688 | 5.127 | 1.00 | 30.39 | S |
| ATOM | 4939 | CE | MET | D | 160 | 19.733 | −8.656 | 4.465 | 1.00 | 30.11 | C |
| ATOM | 4940 | C | MET | D | 160 | 23.913 | −7.454 | 7.980 | 1.00 | 29.85 | C |
| ATOM | 4941 | O | MET | D | 160 | 25.044 | −7.655 | 7.544 | 1.00 | 30.02 | O |
| ATOM | 4942 | N | ALA | D | 161 | 23.472 | −7.986 | 9.119 | 1.00 | 31.84 | N |
| ATOM | 4943 | CA | ALA | D | 161 | 24.273 | −8.945 | 9.898 | 1.00 | 35.04 | C |
| ATOM | 4944 | CB | ALA | D | 161 | 24.201 | −8.628 | 11.389 | 1.00 | 34.40 | C |
| ATOM | 4945 | C | ALA | D | 161 | 23.842 | −10.393 | 9.655 | 1.00 | 37.37 | C |
| ATOM | 4946 | O | ALA | D | 161 | 22.659 | −10.662 | 9.445 | 1.00 | 38.74 | O |
| ATOM | 4947 | N | ILE | D | 162 | 24.810 | −11.309 | 9.641 | 1.00 | 41.08 | N |
| ATOM | 4948 | CA | ILE | D | 162 | 24.551 | −12.757 | 9.645 | 1.00 | 44.36 | C |
| ATOM | 4949 | CB | ILE | D | 162 | 24.596 | −13.390 | 8.231 | 1.00 | 45.96 | C |
| ATOM | 4950 | CG1 | ILE | D | 162 | 25.975 | −13.254 | 7.576 | 1.00 | 47.64 | C |
| ATOM | 4951 | CD1 | ILE | D | 162 | 26.132 | −14.158 | 6.368 | 1.00 | 48.79 | C |
| ATOM | 4952 | CG2 | ILE | D | 162 | 23.531 | −12.777 | 7.331 | 1.00 | 47.40 | C |
| ATOM | 4953 | C | ILE | D | 162 | 25.577 | −13.460 | 10.532 | 1.00 | 45.91 | C |
| ATOM | 4954 | O | ILE | D | 162 | 26.677 | −12.946 | 10.754 | 1.00 | 46.94 | O |
| ATOM | 4955 | N | TYR | D | 163 | 25.218 | −14.648 | 11.018 | 1.00 | 47.19 | N |
| ATOM | 4956 | CA | TYR | D | 163 | 26.147 | −15.471 | 11.790 | 1.00 | 49.30 | C |
| ATOM | 4957 | CB | TYR | D | 163 | 25.402 | −16.496 | 12.644 | 1.00 | 48.51 | C |
| ATOM | 4958 | CG | TYR | D | 163 | 24.531 | −15.853 | 13.704 | 1.00 | 48.39 | C |
| ATOM | 4959 | CD1 | TYR | D | 163 | 25.092 | −15.095 | 14.727 | 1.00 | 48.36 | C |
| ATOM | 4960 | CE1 | TYR | D | 163 | 24.306 | −14.498 | 15.700 | 1.00 | 48.61 | C |
| ATOM | 4961 | CZ | TYR | D | 163 | 22.933 | −14.660 | 15.664 | 1.00 | 49.12 | C |
| ATOM | 4962 | OH | TYR | D | 163 | 22.140 | −14.082 | 16.626 | 1.00 | 50.13 | O |
| ATOM | 4963 | CE2 | TYR | D | 163 | 22.350 | −15.408 | 14.666 | 1.00 | 49.21 | C |
| ATOM | 4964 | CD2 | TYR | D | 163 | 23.148 | −16.005 | 13.689 | 1.00 | 49.24 | C |
| ATOM | 4965 | C | TYR | D | 163 | 27.147 | −16.157 | 10.863 | 1.00 | 52.08 | C |
| ATOM | 4966 | O | TYR | D | 163 | 26.787 | −16.589 | 9.783 | 1.00 | 50.54 | O |
| ATOM | 4967 | N | LYS | D | 164 | 28.409 | −16.229 | 11.296 | 1.00 | 57.22 | N |
| ATOM | 4968 | CA | LYS | D | 164 | 29.496 | −16.783 | 10.482 | 1.00 | 60.75 | C |
| ATOM | 4969 | CB | LYS | D | 164 | 30.851 | −16.297 | 11.010 | 1.00 | 61.84 | C |
| ATOM | 4970 | CG | LYS | D | 164 | 32.094 | −16.695 | 10.226 | 1.00 | 61.84 | C |
| ATOM | 4971 | CD | LYS | D | 164 | 33.337 | −16.069 | 10.869 | 1.00 | 61.07 | C |
| ATOM | 4972 | CE | LYS | D | 164 | 34.619 | −16.607 | 10.247 | 1.00 | 60.00 | C |
| ATOM | 4973 | NZ | LYS | D | 164 | 35.815 | −16.424 | 11.112 | 1.00 | 59.50 | N |
| ATOM | 4974 | C | LYS | D | 164 | 29.471 | −18.317 | 10.418 | 1.00 | 64.23 | C |
| ATOM | 4975 | O | LYS | D | 164 | 29.743 | −18.883 | 9.366 | 1.00 | 64.66 | O |
| ATOM | 4976 | N | GLN | D | 165 | 29.161 | −18.980 | 11.539 | 1.00 | 69.27 | N |
| ATOM | 4977 | CA | GLN | D | 165 | 29.279 | −20.444 | 11.610 | 1.00 | 74.57 | C |
| ATOM | 4978 | CB | GLN | D | 165 | 29.263 | −20.920 | 13.059 | 1.00 | 77.00 | C |
| ATOM | 4979 | CG | GLN | D | 165 | 30.425 | −20.421 | 13.885 | 1.00 | 77.94 | C |
| ATOM | 4980 | CD | GLN | D | 165 | 30.378 | −20.846 | 15.342 | 1.00 | 78.56 | C |
| ATOM | 4981 | OE1 | GLN | D | 165 | 29.352 | −21.328 | 15.851 | 1.00 | 77.23 | O |
| ATOM | 4982 | NE2 | GLN | D | 165 | 31.489 | −20.661 | 16.030 | 1.00 | 80.62 | N |
| ATOM | 4983 | C | GLN | D | 165 | 28.163 | −21.121 | 10.809 | 1.00 | 78.01 | C |
| ATOM | 4984 | O | GLN | D | 165 | 26.998 | −20.782 | 10.978 | 1.00 | 79.49 | O |
| ATOM | 4985 | N | SER | D | 166 | 28.536 | −22.089 | 9.972 | 1.00 | 81.59 | N |
| ATOM | 4986 | CA | SER | D | 166 | 27.610 | −22.733 | 9.023 | 1.00 | 83.47 | C |
| ATOM | 4987 | CB | SER | D | 166 | 28.339 | −23.810 | 8.203 | 1.00 | 82.59 | C |
| ATOM | 4988 | OG | SER | D | 166 | 29.077 | −24.687 | 9.041 | 1.00 | 81.58 | O |
| ATOM | 4989 | C | SER | D | 166 | 26.349 | −23.325 | 9.665 | 1.00 | 85.49 | C |
| ATOM | 4990 | O | SER | D | 166 | 25.276 | −23.286 | 9.066 | 1.00 | 84.61 | O |
| ATOM | 4991 | N | GLN | D | 167 | 26.481 | −23.863 | 10.875 | 1.00 | 89.77 | N |
| ATOM | 4992 | CA | GLN | D | 167 | 25.333 | −24.429 | 11.605 | 1.00 | 92.09 | C |
| ATOM | 4993 | CB | GLN | D | 167 | 25.796 | −25.379 | 12.728 | 1.00 | 92.57 | C |
| ATOM | 4994 | CG | GLN | D | 167 | 26.414 | −24.738 | 13.970 | 1.00 | 92.42 | C |
| ATOM | 4995 | CD | GLN | D | 167 | 27.898 | −24.418 | 13.846 | 1.00 | 92.91 | C |
| ATOM | 4996 | OE1 | GLN | D | 167 | 28.462 | −24.405 | 12.742 | 1.00 | 92.30 | O |
| ATOM | 4997 | NE2 | GLN | D | 167 | 28.539 | −24.154 | 14.980 | 1.00 | 93.17 | N |
| ATOM | 4998 | C | GLN | D | 167 | 24.332 | −23.382 | 12.152 | 1.00 | 94.23 | C |
| ATOM | 4999 | O | GLN | D | 167 | 23.202 | −23.738 | 12.481 | 1.00 | 95.90 | O |
| ATOM | 5000 | N | HIS | D | 168 | 24.763 | −22.121 | 12.272 | 1.00 | 93.54 | N |
| ATOM | 5001 | CA | HIS | D | 168 | 23.898 | −21.021 | 12.709 | 1.00 | 91.47 | C |
| ATOM | 5002 | CB | HIS | D | 168 | 24.563 | −20.250 | 13.873 | 1.00 | 94.04 | C |
| ATOM | 5003 | CG | HIS | D | 168 | 24.771 | −2.051 | 15.127 | 1.00 | 97.15 | C |
| ATOM | 5004 | ND1 | HIS | D | 168 | 25.964 | −21.686 | 15.404 | 1.00 | 99.32 | N |
| ATOM | 5005 | CE1 | HIS | D | 168 | 25.898 | −22.250 | 16.596 | 1.00 | 100.47 | C |
| ATOM | 5006 | NE2 | HIS | D | 168 | 24.743 | −21.924 | 17.148 | 1.00 | 99.61 | N |
| ATOM | 5007 | CD2 | HIS | D | 168 | 24.039 | −21.133 | 16.279 | 1.00 | 97.82 | C |
| ATOM | 5008 | C | HIS | D | 168 | 23.505 | −20.006 | 11.631 | 1.00 | 87.94 | C |
| ATOM | 5009 | O | HIS | D | 168 | 22.679 | −19.114 | 11.891 | 1.00 | 87.90 | O |
| ATOM | 5010 | N | MET | D | 169 | 24.054 | −20.148 | 10.421 | 1.00 | 83.05 | N |
| ATOM | 5011 | CA | MET | D | 169 | 23.890 | −19.137 | 9.348 | 1.00 | 79.87 | C |
| ATOM | 5012 | CB | MET | D | 169 | 24.678 | −19.545 | 8.088 | 1.00 | 79.32 | C |
| ATOM | 5013 | CG | MET | D | 169 | 24.898 | −18.401 | 7.118 | 1.00 | 79.41 | C |
| ATOM | 5014 | SD | MET | D | 169 | 26.196 | −18.757 | 5.888 | 1.00 | 78.11 | S |
| ATOM | 5015 | CE | MET | D | 169 | 27.770 | −18.914 | 6.784 | 1.00 | 78.42 | C |
| ATOM | 5016 | C | MET | D | 169 | 22.446 | −18.821 | 8.954 | 1.00 | 77.34 | C |
| ATOM | 5017 | O | MET | D | 169 | 22.153 | −17.712 | 8.506 | 1.00 | 76.81 | O |
| ATOM | 5018 | N | THR | D | 170 | 21.548 | −19.795 | 9.111 | 1.00 | 75.50 | N |
| ATOM | 5019 | CA | THR | D | 170 | 20.134 | −19.633 | 8.759 | 1.00 | 72.56 | C |
| ATOM | 5020 | CB | THR | D | 170 | 19.466 | −20.998 | 8.484 | 1.00 | 72.33 | C |
| ATOM | 5021 | OG1 | THR | D | 170 | 19.497 | −21.812 | 9.663 | 1.00 | 71.55 | O |
| ATOM | 5022 | CG2 | THR | D | 170 | 20.182 | −21.731 | 7.369 | 1.00 | 73.47 | C |
| ATOM | 5023 | C | THR | D | 170 | 19.320 | −18.886 | 9.820 | 1.00 | 69.19 | C |
| ATOM | 5024 | O | THR | D | 170 | 18.224 | −18.414 | 9.514 | 1.00 | 65.83 | O |
| ATOM | 5025 | N | GLU | D | 171 | 19.837 | −18.786 | 11.049 | 1.00 | 66.89 | N |
| ATOM | 5026 | CA | GLU | D | 171 | 19.164 | −18.028 | 12.106 | 1.00 | 66.83 | C |
| ATOM | 5027 | CB | GLU | D | 171 | 19.730 | −18.363 | 13.483 | 1.00 | 68.87 | C |
| ATOM | 5028 | CG | GLU | D | 171 | 19.551 | −19.819 | 13.888 | 1.00 | 69.82 | C |
| ATOM | 5029 | CD | GLU | D | 171 | 19.807 | −20.081 | 15.364 | 1.00 | 70.44 | C |
| ATOM | 5030 | OE1 | GLU | D | 171 | 19.080 | −20.937 | 15.908 | 1.00 | 68.93 | O |
| ATOM | 5031 | OE2 | GLU | D | 171 | 20.709 | −19.473 | 16.004 | 1.00 | 69.53 | O |
| ATOM | 5032 | C | GLU | D | 171 | 19.278 | −16.520 | 11.866 | 1.00 | 65.44 | C |
| ATOM | 5033 | O | GLU | D | 171 | 20.321 | −16.035 | 11.427 | 1.00 | 65.55 | O |
| ATOM | 5034 | N | VAL | D | 172 | 18.213 | −15.801 | 12.187 | 1.00 | 63.65 | N |
| ATOM | 5035 | CA | VAL | D | 172 | 18.172 | −14.355 | 11.998 | 1.00 | 62.38 | C |
| ATOM | 5036 | CB | VAL | D | 172 | 16.723 | −13.808 | 11.986 | 1.00 | 63.36 | C |
| ATOM | 5037 | CG1 | VAL | D | 172 | 16.727 | −12.300 | 11.811 | 1.00 | 62.41 | C |
| ATOM | 5038 | CG2 | VAL | D | 172 | 15.917 | −14.428 | 10.863 | 1.00 | 64.32 | C |
| ATOM | 5039 | C | VAL | D | 172 | 18.949 | −13.679 | 13.121 | 1.00 | 60.37 | C |
| ATOM | 5040 | O | VAL | D | 172 | 18.672 | −13.913 | 14.286 | 1.00 | 58.55 | O |
| ATOM | 5041 | N | VAL | D | 173 | 19.921 | −12.846 | 12.757 | 1.00 | 58.54 | N |
| ATOM | 5042 | CA | VAL | D | 173 | 20.649 | −12.044 | 13.735 | 1.00 | 57.96 | C |
| ATOM | 5043 | CB | VAL | D | 173 | 21.935 | −11.408 | 13.151 | 1.00 | 57.53 | C |
| ATOM | 5044 | CG1 | VAL | D | 173 | 22.569 | −10.458 | 14.161 | 1.00 | 57.69 | C |
| ATOM | 5045 | CG2 | VAL | D | 173 | 22.927 | −12.498 | 12.731 | 1.00 | 57.15 | C |
| ATOM | 5046 | C | VAL | D | 173 | 19.699 | −10.929 | 14.178 | 1.00 | 57.65 | C |
| ATOM | 5047 | O | VAL | D | 173 | 19.311 | −10.070 | 13.372 | 1.00 | 58.17 | O |
| ATOM | 5048 | N | ARG | D | 174 | 19.315 | −10.961 | 15.455 | 1.00 | 56.74 | N |
| ATOM | 5049 | CA | ARG | D | 174 | 18.566 | −9.860 | 16.069 | 1.00 | 58.03 | C |
| ATOM | 5050 | CB | ARG | D | 174 | 17.156 | −10.317 | 16.448 | 1.00 | 59.47 | C |
| ATOM | 5051 | CG | ARG | D | 174 | 16.354 | −11.199 | 15.485 | 1.00 | 61.81 | C |
| ATOM | 5052 | CD | ARG | D | 174 | 15.156 | −11.809 | 16.221 | 1.00 | 63.38 | C |
| ATOM | 5053 | NE | ARG | D | 174 | 14.220 | −12.514 | 15.333 | 1.00 | 64.18 | N |
| ATOM | 5054 | CZ | ARG | D | 174 | 14.210 | −13.827 | 15.074 | 1.00 | 64.76 | C |
| ATOM | 5055 | NH1 | ARG | D | 174 | 15.096 | −14.669 | 15.615 | 1.00 | 64.92 | N |
| ATOM | 5056 | NH2 | ARG | D | 174 | 13.296 | −14.316 | 14.233 | 1.00 | 65.93 | N |
| ATOM | 5057 | C | ARG | D | 174 | 19.309 | −9.383 | 17.318 | 1.00 | 58.64 | C |
| ATOM | 5058 | O | ARG | D | 174 | 20.225 | −10.055 | 17.810 | 1.00 | 57.48 | O |
| ATOM | 5059 | N | ARG | D | 175 | 18.914 | −8.222 | 17.806 | 1.00 | 60.06 | N |
| ATOM | 5060 | CA | ARG | D | 175 | 19.469 | −7.663 | 19.050 | 1.00 | 63.09 | C |
| ATOM | 5061 | CB | ARG | D | 175 | 18.990 | −6.229 | 19.223 | 1.00 | 62.76 | C |
| ATOM | 5062 | CG | ARG | D | 175 | 19.734 | −5.393 | 20.225 | 1.00 | 62.50 | C |
| ATOM | 5063 | CD | ARG | D | 175 | 19.156 | −4.003 | 20.129 | 1.00 | 62.22 | C |
| ATOM | 5064 | NE | ARG | D | 175 | 19.555 | −3.090 | 21.206 | 1.00 | 63.20 | N |
| ATOM | 5065 | CZ | ARG | D | 175 | 18.840 | −2.027 | 21.618 | 1.00 | 63.46 | C |
| ATOM | 5066 | NH1 | ARG | D | 175 | 19.326 | −1.281 | 22.608 | 1.00 | 62.81 | N |
| ATOM | 5067 | NH2 | ARG | D | 175 | 17.674 | −1.701 | 21.057 | 1.00 | 62.50 | N |
| ATOM | 5068 | C | ARG | D | 175 | 19.033 | −8.516 | 20.248 | 1.00 | 68.16 | C |
| ATOM | 5069 | O | ARG | D | 175 | 17.962 | −9.150 | 20.211 | 1.00 | 68.31 | O |
| ATOM | 5070 | N | CYS | D | 176 | 19.858 | −8.540 | 21.289 | 1.00 | 72.94 | N |
| ATOM | 5071 | CA | CYS | D | 176 | 19.522 | −9.277 | 22.520 | 1.00 | 75.55 | C |
| ATOM | 5072 | CB | CYS | D | 176 | 20.685 | −9.258 | 23.523 | 1.00 | 75.83 | C |
| ATOM | 5073 | SG | CYS | D | 176 | 21.156 | −7.610 | 24.137 | 1.00 | 73.32 | S |
| ATOM | 5074 | C | CYS | D | 176 | 18.237 | −8.726 | 23.172 | 1.00 | 79.88 | C |
| ATOM | 5075 | O | CYS | D | 176 | 18.027 | −7.499 | 23.221 | 1.00 | 79.96 | O |
| ATOM | 5076 | N | PRO | D | 177 | 17.276 | −9.627 | 23.513 | 1.00 | 82.93 | N |
| ATOM | 5077 | CA | PRO | D | 177 | 15.992 | −9.133 | 24.021 | 1.00 | 82.86 | C |
| ATOM | 5078 | CB | PRO | D | 177 | 15.222 | −10.420 | 24.299 | 1.00 | 82.32 | C |
| ATOM | 5079 | CG | PRO | D | 177 | 15.704 | −11.349 | 23.235 | 1.00 | 82.18 | C |
| ATOM | 5080 | CD | PRO | D | 177 | 17.162 | −11.024 | 23.058 | 1.00 | 81.94 | C |
| ATOM | 5081 | C | PRO | D | 177 | 16.129 | −8.278 | 25.278 | 1.00 | 81.91 | C |
| ATOM | 5082 | O | PRO | D | 177 | 15.431 | −7.266 | 25.418 | 1.00 | 78.54 | O |
| ATOM | 5083 | N | LEU | D | 188 | 13.737 | 6.644 | 15.159 | 1.00 | 65.75 | N |
| ATOM | 5084 | CA | LEU | D | 188 | 13.677 | 6.077 | 13.827 | 1.00 | 66.96 | C |
| ATOM | 5085 | CB | LEU | D | 188 | 14.685 | 6.787 | 12.917 | 1.00 | 68.40 | C |
| ATOM | 5086 | CG | LEU | D | 188 | 14.486 | 8.289 | 12.694 | 1.00 | 70.40 | C |
| ATOM | 5087 | CD1 | LEU | D | 188 | 15.692 | 8.848 | 11.946 | 1.00 | 70.69 | C |
| ATOM | 5088 | CD2 | LEU | D | 188 | 13.208 | 8.601 | 11.923 | 1.00 | 69.55 | C |
| ATOM | 5089 | C | LEU | D | 188 | 13.936 | 4.568 | 13.800 | 1.00 | 64.65 | C |
| ATOM | 5090 | O | LEU | D | 188 | 13.250 | 3.849 | 13.067 | 1.00 | 66.02 | O |
| ATOM | 5091 | N | ALA | D | 189 | 14.900 | 4.090 | 14.590 | 1.00 | 61.27 | N |
| ATOM | 5092 | CA | ALA | D | 189 | 15.368 | 2.703 | 14.485 | 1.00 | 59.65 | C |
| ATOM | 5093 | CB | ALA | D | 189 | 16.799 | 2.557 | 15.028 | 1.00 | 59.86 | C |
| ATOM | 5094 | C | ALA | D | 189 | 14.455 | 1.716 | 15.223 | 1.00 | 58.51 | C |
| ATOM | 5095 | O | ALA | D | 189 | 14.020 | 1.997 | 16.344 | 1.00 | 60.12 | O |
| ATOM | 5096 | N | PRO | D | 190 | 14.185 | 0.547 | 14.615 | 1.00 | 57.34 | N |
| ATOM | 5097 | CA | PRO | D | 190 | 13.477 | −0.522 | 15.331 | 1.00 | 55.53 | C |
| ATOM | 5098 | CB | PRO | D | 190 | 13.356 | −1.645 | 14.290 | 1.00 | 55.12 | C |
| ATOM | 5099 | CG | PRO | D | 190 | 13.579 | −0.996 | 12.962 | 1.00 | 56.24 | C |
| ATOM | 5100 | CD | PRO | D | 190 | 14.416 | 0.222 | 13.194 | 1.00 | 56.22 | C |
| ATOM | 5101 | C | PRO | D | 190 | 14.275 | −1.033 | 16.528 | 1.00 | 54.62 | C |
| ATOM | 5102 | O | PRO | D | 190 | 15.505 | −1.104 | 16.447 | 1.00 | 54.24 | O |
| ATOM | 5103 | N | PRO | D | 191 | 13.590 | −1.404 | 17.626 | 1.00 | 54.46 | N |
| ATOM | 5104 | CA | PRO | D | 191 | 14.319 | −1.895 | 18.811 | 1.00 | 54.32 | C |
| ATOM | 5105 | CB | PRO | D | 191 | 13.236 | −1.968 | 19.903 | 1.00 | 55.47 | C |
| ATOM | 5106 | CG | PRO | D | 191 | 11.927 | −1.989 | 19.178 | 1.00 | 56.09 | C |
| ATOM | 5107 | CD | PRO | D | 191 | 12.127 | −1.328 | 17.839 | 1.00 | 56.18 | C |
| ATOM | 5108 | C | PRO | D | 191 | 15.028 | −3.253 | 18.631 | 1.00 | 52.20 | C |
| ATOM | 5109 | O | PRO | D | 191 | 15.945 | −3.547 | 19.379 | 1.00 | 52.94 | O |
| ATOM | 5110 | N | GLN | D | 192 | 14.580 | −4.079 | 17.685 | 1.00 | 50.15 | N |
| ATOM | 5111 | CA | GLN | D | 192 | 15.141 | −5.427 | 17.488 | 1.00 | 47.81 | C |
| ATOM | 5112 | CB | GLN | D | 192 | 14.088 | −6.399 | 16.907 | 1.00 | 49.06 | C |
| ATOM | 5113 | CG | GLN | D | 192 | 12.885 | −6.678 | 17.816 | 1.00 | 50.96 | C |
| ATOM | 5114 | CD | GLN | D | 192 | 12.373 | −8.114 | 17.738 | 1.00 | 52.69 | C |
| ATOM | 5115 | OE1 | GLN | D | 192 | 13.160 | −9.028 | 17.514 | 1.00 | 52.68 | O |
| ATOM | 5116 | NE2 | GLN | D | 192 | 11.075 | −8.305 | 17.932 | 1.00 | 53.35 | N |
| ATOM | 5117 | C | GLN | D | 192 | 16.376 | −5.495 | 16.597 | 1.00 | 44.86 | C |
| ATOM | 5118 | O | GLN | D | 192 | 17.085 | −6.519 | 16.592 | 1.00 | 44.34 | O |
| ATOM | 5119 | N | HIS | D | 193 | 16.682 | −4.427 | 15.851 | 1.00 | 41.20 | N |
| ATOM | 5120 | CA | HIS | D | 193 | 17.817 | −4.405 | 14.904 | 1.00 | 38.05 | C |
| ATOM | 5121 | CB | HIS | D | 193 | 17.655 | −3.268 | 13.887 | 1.00 | 36.35 | C |
| ATOM | 5122 | CG | HIS | D | 193 | 16.645 | −3.543 | 12.814 | 1.00 | 35.34 | C |
| ATOM | 5123 | ND1 | HIS | D | 193 | 16.829 | −3.193 | 11.488 | 1.00 | 35.28 | N |
| ATOM | 5124 | CE1 | HIS | D | 193 | 15.761 | −3.545 | 10.789 | 1.00 | 34.70 | C |
| ATOM | 5125 | NE2 | HIS | D | 193 | 14.898 | −4.116 | 11.612 | 1.00 | 34.57 | N |
| ATOM | 5126 | CD2 | HIS | D | 193 | 15.427 | −4.131 | 12.880 | 1.00 | 34.68 | C |
| ATOM | 5127 | C | HIS | D | 193 | 19.150 | −4.250 | 15.620 | 1.00 | 36.06 | C |
| ATOM | 5128 | O | HIS | D | 193 | 19.322 | −3.358 | 16.460 | 1.00 | 35.74 | O |
| ATOM | 5129 | N | LEU | D | 194 | 20.099 | −5.107 | 15.258 | 1.00 | 35.60 | N |
| ATOM | 5130 | CA | LEU | D | 194 | 21.498 | −4.985 | 15.696 | 1.00 | 36.62 | C |
| ATOM | 5131 | CB | LEU | D | 194 | 22.310 | −6.211 | 15.265 | 1.00 | 37.24 | C |
| ATOM | 5132 | CG | LEU | D | 194 | 23.811 | −6.238 | 15.607 | 1.00 | 38.07 | C |
| ATOM | 5133 | CD1 | LEU | D | 194 | 24.092 | −6.120 | 17.094 | 1.00 | 39.19 | C |
| ATOM | 5134 | CD2 | LEU | D | 194 | 24.443 | −7.547 | 15.145 | 1.00 | 38.38 | C |
| ATOM | 5135 | C | LEU | D | 194 | 22.164 | −3.720 | 15.157 | 1.00 | 35.45 | C |
| ATOM | 5136 | O | LEU | D | 194 | 22.794 | −2.992 | 15.907 | 1.00 | 37.37 | O |
| ATOM | 5137 | N | ILE | D | 195 | 22.035 | −3.478 | 13.840 | 1.00 | 33.67 | N |
| ATOM | 5138 | CA | ILE | D | 195 | 22.783 | −2.406 | 13.180 | 1.00 | 33.87 | C |
| ATOM | 5139 | CB | ILE | D | 195 | 23.398 | −2.863 | 11.828 | 1.00 | 34.00 | C |
| ATOM | 5140 | CG1 | ILE | D | 195 | 24.177 | −4.170 | 12.008 | 1.00 | 34.58 | C |
| ATOM | 5141 | CD1 | ILE | D | 195 | 24.573 | −4.871 | 10.738 | 1.00 | 34.75 | C |
| ATOM | 5142 | CG2 | ILE | D | 195 | 24.335 | −1.792 | 11.282 | 1.00 | 34.16 | C |
| ATOM | 5143 | C | ILE | D | 195 | 21.910 | −1.183 | 12.955 | 1.00 | 33.54 | C |
| ATOM | 5144 | O | ILE | D | 195 | 20.849 | −1.282 | 12.347 | 1.00 | 33.02 | O |
| ATOM | 5145 | N | ARG | D | 196 | 22.376 | −0.035 | 13.444 | 1.00 | 34.46 | N |
| ATOM | 5146 | CA | ARG | D | 196 | 21.767 | 1.266 | 13.158 | 1.00 | 36.60 | C |
| ATOM | 5147 | CB | ARG | D | 196 | 21.404 | 1.988 | 14.446 | 1.00 | 38.40 | C |
| ATOM | 5148 | CG | ARG | D | 196 | 20.393 | 1.254 | 15.299 | 1.00 | 41.84 | C |
| ATOM | 5149 | CD | ARG | D | 196 | 20.074 | 2.025 | 16.564 | 1.00 | 43.59 | C |
| ATOM | 5150 | NE | ARG | D | 196 | 19.132 | 1.294 | 17.405 | 1.00 | 46.25 | N |
| ATOM | 5151 | CZ | ARG | D | 196 | 18.662 | 1.716 | 18.582 | 1.00 | 47.11 | C |
| ATOM | 5152 | NH1 | ARG | D | 196 | 19.028 | 2.894 | 19.088 | 1.00 | 47.40 | N |
| ATOM | 5153 | NH2 | ARG | D | 196 | 17.792 | 0.961 | 19.248 | 1.00 | 48.29 | N |
| ATOM | 5154 | C | ARG | D | 196 | 22.748 | 2.132 | 12.398 | 1.00 | 36.20 | C |
| ATOM | 5155 | O | ARG | D | 196 | 23.943 | 1.853 | 12.378 | 1.00 | 35.72 | O |
| ATOM | 5156 | N | VAL | D | 197 | 22.222 | 3.177 | 11.773 | 1.00 | 35.20 | N |
| ATOM | 5157 | CA | VAL | D | 197 | 23.026 | 4.250 | 11.226 | 1.00 | 35.80 | C |
| ATOM | 5158 | CB | VAL | D | 197 | 22.717 | 4.514 | 9.742 | 1.00 | 35.45 | C |
| ATOM | 5159 | CG1 | VAL | D | 197 | 23.450 | 5.765 | 9.242 | 1.00 | 35.06 | C |
| ATOM | 5160 | CG2 | VAL | D | 197 | 23.126 | 3.308 | 8.918 | 1.00 | 35.22 | C |
| ATOM | 5161 | C | VAL | D | 197 | 22.777 | 5.505 | 12.055 | 1.00 | 37.14 | C |
| ATOM | 5162 | O | VAL | D | 197 | 21.641 | 5.830 | 12.383 | 1.00 | 35.62 | O |
| ATOM | 5163 | N | GLU | D | 198 | 23.868 | 6.195 | 12.368 | 1.00 | 39.59 | N |
| ATOM | 5164 | CA | GLU | D | 198 | 23.864 | 7.393 | 13.189 | 1.00 | 42.79 | C |
| ATOM | 5165 | CB | GLU | D | 198 | 25.046 | 7.328 | 14.150 | 1.00 | 46.60 | C |
| ATOM | 5166 | CG | GLU | D | 198 | 25.055 | 8.382 | 15.249 | 1.00 | 50.35 | C |
| ATOM | 5167 | CD | GLU | D | 198 | 26.351 | 8.377 | 16.053 | 1.00 | 54.93 | C |
| ATOM | 5168 | OE1 | GLU | D | 198 | 27.120 | 7.392 | 15.980 | 1.00 | 56.77 | O |
| ATOM | 5169 | OE2 | GLU | D | 198 | 26.600 | 9.368 | 16.771 | 1.00 | 56.98 | O |
| ATOM | 5170 | C | GLU | D | 198 | 23.995 | 8.621 | 12.287 | 1.00 | 41.19 | C |
| ATOM | 5171 | O | GLU | D | 198 | 24.621 | 8.543 | 11.233 | 1.00 | 42.56 | O |
| ATOM | 5172 | N | GLY | D | 199 | 23.391 | 9.732 | 12.702 | 1.00 | 40.03 | N |
| ATOM | 5173 | CA | GLY | D | 199 | 23.556 | 11.024 | 12.026 | 1.00 | 39.50 | C |
| ATOM | 5174 | C | GLY | D | 199 | 23.161 | 11.072 | 10.559 | 1.00 | 39.40 | C |
| ATOM | 5175 | O | GLY | D | 199 | 23.825 | 11.733 | 9.759 | 1.00 | 36.96 | O |
| ATOM | 5176 | N | ASN | D | 200 | 22.090 | 10.362 | 10.204 | 1.00 | 40.34 | N |
| ATOM | 5177 | CA | ASN | D | 200 | 21.562 | 10.379 | 8.832 | 1.00 | 40.28 | C |
| ATOM | 5178 | CB | ASN | D | 200 | 22.175 | 9.251 | 7.985 | 1.00 | 41.19 | C |
| ATOM | 5179 | CG | ASN | D | 200 | 21.933 | 9.429 | 6.491 | 1.00 | 40.63 | C |
| ATOM | 5180 | OD1 | ASN | D | 200 | 20.855 | 9.839 | 6.072 | 1.00 | 41.66 | O |
| ATOM | 5181 | ND2 | ASN | D | 200 | 22.936 | 9.096 | 5.682 | 1.00 | 40.32 | N |
| ATOM | 5182 | C | ASN | D | 200 | 20.036 | 10.292 | 8.859 | 1.00 | 39.79 | C |
| ATOM | 5183 | O | ASN | D | 200 | 19.463 | 9.229 | 9.089 | 1.00 | 39.33 | O |
| ATOM | 5184 | N | LEU | D | 201 | 19.392 | 11.431 | 8.602 | 1.00 | 40.78 | N |
| ATOM | 5185 | CA | LEU | D | 201 | 17.941 | 11.557 | 8.679 | 1.00 | 42.30 | C |
| ATOM | 5186 | CB | LEU | D | 201 | 17.560 | 13.042 | 8.735 | 1.00 | 45.77 | C |
| ATOM | 5187 | CG | LEU | D | 201 | 16.163 | 13.496 | 9.176 | 1.00 | 49.76 | C |
| ATOM | 5188 | CD1 | LEU | D | 201 | 15.494 | 12.744 | 10.339 | 1.00 | 51.36 | C |
| ATOM | 5189 | CD2 | LEU | D | 201 | 16.233 | 14.982 | 9.526 | 1.00 | 50.43 | C |
| ATOM | 5190 | C | LEU | D | 201 | 17.185 | 10.852 | 7.544 | 1.00 | 41.41 | C |
| ATOM | 5191 | O | LEU | D | 201 | 15.982 | 10.633 | 7.664 | 1.00 | 42.58 | O |
| ATOM | 5192 | N | ARG | D | 202 | 17.883 | 10.491 | 6.462 | 1.00 | 39.00 | N |
| ATOM | 5193 | CA | ARG | D | 202 | 17.301 | 9.705 | 5.361 | 1.00 | 39.06 | C |
| ATOM | 5194 | CB | ARG | D | 202 | 17.931 | 10.122 | 4.035 | 1.00 | 36.67 | C |
| ATOM | 5195 | CG | ARG | D | 202 | 17.619 | 11.558 | 1.644 | 1.00 | 38.25 | C |
| ATOM | 5196 | CD | ARG | D | 202 | 18.599 | 12.117 | 2.628 | 1.00 | 39.06 | C |
| ATOM | 5197 | NE | ARG | D | 202 | 18.241 | 11.843 | 1.235 | 1.00 | 39.84 | N |
| ATOM | 5198 | CZ | ARG | D | 202 | 19.065 | 11.978 | 0.184 | 1.00 | 41.98 | C |
| ATOM | 5199 | NH1 | ARG | D | 202 | 20.313 | 12.425 | 0.321 | 1.00 | 42.46 | N |
| ATOM | 5200 | NH2 | ARG | D | 202 | 18.627 | 11.652 | −1.032 | 1.00 | 42.27 | N |
| ATOM | 5201 | C | ARG | D | 202 | 17.417 | 8.171 | 5.554 | 1.00 | 32.22 | C |
| ATOM | 5202 | O | ARG | D | 202 | 17.175 | 7.420 | 4.610 | 1.00 | 31.73 | O |
| ATOM | 5203 | N | VAL | D | 203 | 17.768 | 7.722 | 6.757 | 1.00 | 29.47 | N |
| ATOM | 5204 | CA | VAL | D | 203 | 17.905 | 6.290 | 7.061 | 1.00 | 28.25 | C |
| ATOM | 5205 | CB | VAL | D | 203 | 18.504 | 6.072 | 8.488 | 1.00 | 28.22 | C |
| ATOM | 5206 | CG1 | VAL | D | 203 | 17.503 | 6.452 | 9.586 | 1.00 | 27.39 | C |
| ATOM | 5207 | CG2 | VAL | D | 203 | 18.991 | 4.632 | 8.682 | 1.00 | 28.29 | C |
| ATOM | 5208 | C | VAL | D | 203 | 16.575 | 5.535 | 6.927 | 1.00 | 26.64 | C |
| ATOM | 5209 | O | VAL | D | 203 | 15.511 | 6.068 | 7.244 | 1.00 | 26.70 | O |
| ATOM | 5210 | N | GLU | D | 204 | 16.655 | 4.296 | 6.444 | 1.00 | 25.04 | N |
| ATOM | 5211 | CA | GLU | D | 204 | 15.488 | 3.438 | 6.248 | 1.00 | 24.38 | C |
| ATOM | 5212 | CB | GLU | D | 204 | 15.125 | 3.401 | 4.760 | 1.00 | 24.42 | C |
| ATOM | 5213 | CG | GLU | D | 204 | 14.174 | 2.295 | 4.307 | 1.00 | 24.70 | C |
| ATOM | 5214 | CD | GLU | D | 204 | 14.005 | 2.271 | 2.789 | 1.00 | 25.02 | C |
| ATOM | 5215 | OE1 | GLU | D | 204 | 13.966 | 3.362 | 2.174 | 1.00 | 23.68 | O |
| ATOM | 5216 | OE2 | GLU | D | 204 | 13.928 | 1.155 | 2.209 | 1.00 | 25.69 | O |
| ATOM | 5217 | C | GLU | D | 204 | 15.806 | 2.046 | 6.759 | 1.00 | 24.02 | C |
| ATOM | 5218 | O | GLU | D | 204 | 16.877 | 1.510 | 6.468 | 1.00 | 23.42 | O |
| ATOM | 5219 | N | TYR | D | 205 | 14.861 | 1.461 | 7.495 | 1.00 | 23.41 | N |
| ATOM | 5220 | CA | TYR | D | 205 | 14.998 | 0.105 | 8.032 | 1.00 | 24.13 | C |
| ATOM | 5221 | CB | TYR | D | 205 | 14.803 | 0.125 | 9.553 | 1.00 | 24.06 | C |
| ATOM | 5222 | CG | TYR | D | 205 | 15.903 | 0.878 | 10.255 | 1.00 | 23.91 | C |
| ATOM | 5223 | CD1 | TYR | D | 205 | 17.076 | 0.223 | 10.637 | 1.00 | 23.83 | C |
| ATOM | 5224 | CE1 | TYR | D | 205 | 18.081 | 0.898 | 11.317 | 1.00 | 24.18 | C |
| ATOM | 5225 | CZ | TYR | D | 205 | 17.935 | 2.255 | 11.578 | 1.00 | 23.70 | C |
| ATOM | 5226 | OH | TYR | D | 205 | 18.956 | 2.908 | 12.213 | 1.00 | 23.91 | O |
| ATOM | 5227 | CE2 | TYR | D | 205 | 16.798 | 2.933 | 11.165 | 1.00 | 23.97 | C |
| ATOM | 5228 | CD2 | TYR | D | 205 | 15.781 | 2.237 | 10.537 | 1.00 | 23.77 | C |
| ATOM | 5229 | C | TYR | D | 205 | 14.015 | −0.862 | 7.388 | 1.00 | 25.20 | C |
| ATOM | 5230 | O | TYR | D | 205 | 12.694 | −0.479 | 7.046 | 1.00 | 25.76 | O |
| ATOM | 5231 | N | LEU | D | 206 | 14.445 | −2.116 | 7.256 | 1.00 | 26.15 | N |
| ATOM | 5232 | CA | LEU | D | 206 | 13.656 | −3.176 | 6.622 | 1.00 | 27.21 | C |
| ATOM | 5233 | CB | LEU | D | 206 | 14.218 | −3.506 | 5.231 | 1.00 | 27.43 | C |
| ATOM | 5234 | CG | LEU | D | 206 | 13.531 | −4.586 | 4.370 | 1.00 | 27.81 | C |
| ATOM | 5235 | CD1 | LEU | D | 206 | 12.160 | −4.140 | 3.899 | 1.00 | 27.35 | C |
| ATOM | 5236 | CD2 | LEU | D | 206 | 14.382 | −4.925 | 3.150 | 1.00 | 28.04 | C |
| ATOM | 5237 | C | LEU | D | 206 | 13.667 | −4.432 | 7.488 | 1.00 | 28.59 | C |
| ATOM | 5238 | O | LEU | D | 206 | 14.718 | −4.900 | 7.926 | 1.00 | 27.66 | O |
| ATOM | 5239 | N | ASP | D | 207 | 12.472 | −4.941 | 7.756 | 1.00 | 30.89 | N |
| ATOM | 5240 | CA | ASP | D | 207 | 12.281 | −6.332 | 8.145 | 1.00 | 32.84 | C |
| ATOM | 5241 | CB | ASP | D | 207 | 11.183 | −6.473 | 9.205 | 1.00 | 33.46 | C |
| ATOM | 5242 | CG | ASP | D | 207 | 11.544 | −5.839 | 10.535 | 1.00 | 34.13 | C |
| ATOM | 5243 | OD1 | ASP | D | 207 | 12.724 | −5.502 | 10.758 | 1.00 | 33.91 | O |
| ATOM | 5244 | OD2 | ASP | D | 207 | 10.627 | −5.680 | 11.371 | 1.00 | 37.54 | O |
| ATOM | 5245 | C | ASP | D | 207 | 11.827 | −7.000 | 6.865 | 1.00 | 34.07 | C |
| ATOM | 5246 | O | ASP | D | 207 | 10.762 | −6.675 | 6.343 | 1.00 | 31.51 | O |
| ATOM | 5247 | N | ASP | D | 208 | 12.635 | −7.908 | 6.333 | 1.00 | 38.42 | N |
| ATOM | 5248 | CA | ASP | D | 208 | 12.285 | −8.586 | 5.089 | 1.00 | 42.31 | C |
| ATOM | 5249 | CB | ASP | D | 208 | 13.464 | −9.391 | 4.551 | 1.00 | 42.99 | C |
| ATOM | 5250 | CG | ASP | D | 208 | 13.247 | −9.856 | 3.116 | 1.00 | 43.15 | C |
| ATOM | 5251 | OD1 | ASP | D | 208 | 12.393 | −10.741 | 2.898 | 1.00 | 42.76 | O |
| ATOM | 5252 | OD1 | ASP | D | 208 | 13.938 | −9.333 | 2.211 | 1.00 | 44.14 | O |
| ATOM | 5253 | C | ASP | D | 208 | 11.080 | −9.495 | 5.340 | 1.00 | 46.20 | C |
| ATOM | 5254 | O | ASP | D | 208 | 11.088 | −10.282 | 6.276 | 1.00 | 44.94 | O |
| ATOM | 5255 | N | ARG | D | 209 | 10.049 | −9.365 | 4.505 | 1.00 | 52.61 | N |
| ATOM | 5256 | CA | ARG | D | 209 | 8.794 | −10.107 | 4.709 | 1.00 | 57.91 | C |
| ATOM | 5257 | CB | ARG | D | 209 | 7.660 | −9.483 | 3.874 | 1.00 | 60.88 | C |
| ATOM | 5258 | CG | ARG | D | 209 | 7.794 | −9.663 | 2.363 | 1.00 | 64.29 | C |
| ATOM | 5259 | CD | ARG | D | 209 | 7.303 | −8.394 | 1.660 | 1.00 | 66.04 | C |
| ATOM | 5260 | NE | ARG | D | 209 | 7.428 | −8.426 | 0.207 | 1.00 | 68.47 | N |
| ATOM | 5261 | CZ | ARG | D | 209 | 8.566 | −8.285 | −0.479 | 1.00 | 69.59 | C |
| ATOM | 5262 | NH1 | ARG | D | 209 | 8.525 | −8.306 | −1.803 | 1.00 | 70.74 | N |
| ATOM | 5263 | NH2 | ARG | D | 209 | 9.744 | −8.135 | 0.132 | 1.00 | 70.07 | N |
| ATOM | 5264 | C | ARG | D | 209 | 8.892 | −11.612 | 4.436 | 1.00 | 59.53 | C |
| ATOM | 5265 | O | ARG | D | 209 | 8.076 | −12.374 | 4.956 | 1.00 | 61.28 | O |
| ATOM | 5266 | N | ASN | D | 210 | 9.879 | −12.043 | 3.643 | 1.00 | 58.68 | N |
| ATOM | 5267 | CA | ASN | D | 210 | 10.078 | −13.475 | 3.342 | 1.00 | 58.66 | C |
| ATOM | 5268 | CB | ASN | D | 210 | 10.423 | −13.662 | 1.863 | 1.00 | 58.47 | C |
| ATOM | 5269 | CG | ASN | D | 210 | 9.369 | −13.070 | 0.934 | 1.00 | 58.11 | C |
| ATOM | 5270 | OD1 | ASN | D | 210 | 9.691 | −12.553 | −0.133 | 1.00 | 59.06 | O |
| ATOM | 5271 | OD2 | ASN | D | 210 | 8.105 | −13.153 | 1.334 | 1.00 | 58.67 | N |
| ATOM | 5272 | C | ASN | D | 210 | 11.146 | −14.143 | 4.214 | 1.00 | 58.48 | C |
| ATOM | 5273 | O | ASN | D | 210 | 10.920 | −15.243 | 4.716 | 1.00 | 61.21 | O |
| ATOM | 5274 | N | THR | D | 211 | 12.292 | −13.483 | 4.393 | 1.00 | 57.07 | N |
| ATOM | 5275 | CA | THR | D | 211 | 13.416 | −14.036 | 5.176 | 1.00 | 54.74 | C |
| ATOM | 5276 | CB | THR | D | 211 | 14.779 | −13.637 | 4.566 | 1.00 | 55.26 | C |
| ATOM | 5277 | OG1 | THR | D | 211 | 14.946 | −12.218 | 4.637 | 1.00 | 55.74 | O |
| ATOM | 5278 | CG2 | THR | D | 211 | 14.888 | −14.090 | 3.115 | 1.00 | 55.20 | C |
| ATOM | 5279 | C | THR | D | 211 | 13.438 | −13.605 | 6.645 | 1.00 | 52.78 | C |
| ATOM | 5280 | O | THR | D | 211 | 14.146 | −14.212 | 7.442 | 1.00 | 51.84 | O |
| ATOM | 5281 | N | PHE | D | 212 | 12.699 | −12.542 | 6.976 | 1.00 | 53.59 | N |
| ATOM | 5282 | CA | PHE | D | 212 | 12.676 | −11.941 | 8.332 | 1.00 | 53.96 | C |
| ATOM | 5283 | CB | PHE | D | 212 | 12.191 | −12.945 | 9.396 | 1.00 | 55.80 | C |
| ATOM | 5284 | CG | PHE | D | 212 | 10.964 | −13.709 | 8.988 | 1.00 | 58.10 | C |
| ATOM | 5285 | CD1 | PHE | D | 212 | 9.751 | −13.047 | 8.806 | 1.00 | 58.82 | C |
| ATOM | 5286 | CE1 | PHE | D | 212 | 8.612 | −13.747 | 8.432 | 1.00 | 59.83 | C |
| ATOM | 5287 | CZ | PHE | D | 212 | 8.681 | −15.115 | 8.204 | 1.00 | 59.75 | C |
| ATOM | 5288 | OE2 | PHE | D | 212 | 9.879 | −15.788 | 8.391 | 1.00 | 59.32 | C |
| ATOM | 5289 | CD2 | PHE | D | 212 | 11.015 | −15.089 | 8.777 | 1.00 | 58.96 | C |
| ATOM | 5290 | C | PHE | D | 212 | 13.995 | −11.298 | 8.766 | 1.00 | 52.09 | C |
| ATOM | 5291 | O | PHE | D | 212 | 14.138 | −10.929 | 9.927 | 1.00 | 52.41 | O |
| ATOM | 5292 | N | ARG | D | 213 | 14.931 | −11.118 | 7.833 | 1.00 | 48.46 | N |
| ATOM | 5293 | CA | ARG | D | 213 | 16.235 | −10.546 | 8.169 | 1.00 | 46.88 | C |
| ATOM | 5294 | CB | ARG | D | 213 | 17.317 | −11.026 | 7.201 | 1.00 | 48.48 | C |
| ATOM | 5295 | CG | ARG | D | 213 | 17.640 | −12.507 | 7.397 | 1.00 | 50.02 | C |
| ATOM | 5296 | CD | ARG | D | 213 | 18.810 | −12.932 | 6.545 | 1.00 | 51.13 | C |
| ATOM | 5297 | NE | ARG | D | 213 | 19.070 | −14.370 | 6.667 | 1.00 | 51.98 | N |
| ATOM | 5298 | CZ | ARG | D | 213 | 19.849 | −14.956 | 7.566 | 1.00 | 54.01 | C |
| ATOM | 5299 | NH1 | ARG | D | 213 | 20.408 | −14.268 | 8.571 | 1.00 | 55.43 | N |
| ATOM | 5300 | NH2 | ARG | D | 213 | 20.007 | −16.280 | 7.515 | 1.00 | 54.50 | N |
| ATOM | 5301 | C | ARG | D | 213 | 16.154 | −9.024 | 8.198 | 1.00 | 43.39 | C |
| ATOM | 5302 | O | ARG | D | 213 | 15.330 | −8.403 | 7.511 | 1.00 | 41.51 | O |
| ATOM | 5303 | N | HIS | D | 214 | 17.004 | −8.437 | 9.034 | 1.00 | 39.79 | N |
| ATOM | 5304 | CA | HIS | D | 214 | 16.997 | −7.010 | 9.312 | 1.00 | 37.33 | C |
| ATOM | 5305 | CB | HIS | D | 214 | 17.221 | −6.764 | 10.804 | 1.00 | 39.00 | C |
| ATOM | 5306 | CG | HIS | D | 214 | 16.158 | −7.357 | 11.678 | 1.00 | 41.26 | C |
| ATOM | 5307 | ND1 | HIS | D | 214 | 16.375 | −7.670 | 13.003 | 1.00 | 43.19 | N |
| ATOM | 5308 | CE1 | HIS | D | 214 | 15.273 | −8.184 | 13.520 | 1.00 | 41.74 | C |
| ATOM | 5309 | NE2 | HIS | D | 214 | 14.350 | −8.215 | 12.578 | 1.00 | 42.66 | N |
| ATOM | 5310 | CD2 | HIS | D | 214 | 14.881 | −7.718 | 11.412 | 1.00 | 41.39 | C |
| ATOM | 5311 | C | HIS | D | 214 | 18.081 | −6.331 | 8.505 | 1.00 | 33.16 | C |
| ATOM | 5312 | O | HIS | D | 214 | 19.163 | −6.871 | 8.344 | 1.00 | 31.79 | O |
| ATOM | 5313 | N | SER | D | 215 | 17.784 | −5.147 | 7.991 | 1.00 | 30.69 | N |
| ATOM | 5314 | CA | SER | D | 215 | 18.804 | −4.339 | 7.334 | 1.00 | 29.74 | C |
| ATOM | 5315 | CB | SER | D | 215 | 18.935 | −4.727 | 5.859 | 1.00 | 29.53 | C |
| ATOM | 5316 | OG | SER | D | 215 | 17.743 | −4.487 | 5.143 | 1.00 | 30.05 | O |
| ATOM | 5317 | C | SER | D | 215 | 18.519 | −2.852 | 7.482 | 1.00 | 28.00 | C |
| ATOM | 5318 | O | SER | D | 215 | 17.407 | −2.450 | 7.821 | 1.00 | 27.24 | O |
| ATOM | 5319 | N | VAL | D | 216 | 19.546 | −2.051 | 7.236 | 1.00 | 26.17 | N |
| ATOM | 5320 | CA | VAL | D | 216 | 19.454 | −0.599 | 7.310 | 1.00 | 25.64 | C |
| ATOM | 5321 | CB | VAL | D | 216 | 20.107 | −0.039 | 8.601 | 1.00 | 25.15 | C |
| ATOM | 5322 | CG1 | VAL | D | 216 | 21.575 | −0.456 | 8.732 | 1.00 | 24.88 | C |
| ATOM | 5323 | CG2 | VAL | D | 216 | 19.946 | 1.472 | 8.671 | 1.00 | 25.26 | C |
| ATOM | 5324 | C | VAL | D | 216 | 20.092 | −0.023 | 6.047 | 1.00 | 25.86 | C |
| ATOM | 5325 | O | VAL | D | 216 | 21.194 | −0.428 | 5.659 | 1.00 | 24.87 | O |
| ATOM | 5326 | N | VAL | D | 217 | 19.391 | 0.908 | 5.405 | 1.00 | 25.50 | N |
| ATOM | 5327 | CA | VAL | D | 217 | 19.827 | 1.445 | 4.121 | 1.00 | 25.92 | C |
| ATOM | 5328 | CB | VAL | D | 217 | 18.985 | 0.865 | 2.947 | 1.00 | 25.49 | C |
| ATOM | 5329 | CG1 | VAL | D | 217 | 17.500 | 1.169 | 3.088 | 1.00 | 25.97 | C |
| ATOM | 5330 | CG2 | VAL | D | 217 | 19.486 | 1.370 | 1.602 | 1.00 | 25.30 | C |
| ATOM | 5331 | C | VAL | D | 217 | 19.832 | 2.979 | 4.158 | 1.00 | 26.70 | C |
| ATOM | 5332 | O | VAL | D | 217 | 18.974 | 3.595 | 4.796 | 1.00 | 26.62 | O |
| ATOM | 5333 | N | VAL | D | 218 | 20.838 | 3.577 | 3.516 | 1.00 | 27.53 | N |
| ATOM | 5334 | CA | VAL | D | 218 | 20.923 | 5.033 | 3.341 | 1.00 | 27.98 | C |
| ATOM | 5335 | CB | VAL | D | 218 | 21.987 | 5.706 | 4.248 | 1.00 | 28.30 | C |
| ATOM | 5336 | CG1 | VAL | D | 218 | 21.563 | 5.634 | 5.721 | 1.00 | 28.44 | C |
| ATOM | 5337 | CG2 | VAL | D | 218 | 23.370 | 5.084 | 4.060 | 1.00 | 28.96 | C |
| ATOM | 5338 | C | VAL | D | 218 | 21.248 | 5.343 | 1.883 | 1.00 | 28.34 | C |
| ATOM | 5339 | O | VAL | D | 218 | 21.755 | 4.472 | 1.163 | 1.00 | 27.68 | O |
| ATOM | 5340 | N | PRO | D | 219 | 20.945 | 6.575 | 1.426 | 1.00 | 29.61 | N |
| ATOM | 5341 | CA | PRO | D | 219 | 21.384 | 6.963 | 0.087 | 1.00 | 29.95 | C |
| ATOM | 5342 | CB | PRO | D | 219 | 20.745 | 8.347 | −0.120 | 1.00 | 29.87 | C |
| ATOM | 5343 | CG | PRO | D | 219 | 19.606 | 8.377 | 0.831 | 1.00 | 29.85 | C |
| ATOM | 5344 | CD | PRO | D | 219 | 20.050 | 7.586 | 2.015 | 1.00 | 29.64 | C |
| ATOM | 5345 | C | PRO | D | 219 | 22.905 | 7.051 | −0.004 | 1.00 | 31.72 | C |
| ATOM | 5346 | O | PRO | D | 219 | 23.543 | 7.586 | 0.894 | 1.00 | 30.58 | O |
| ATOM | 5347 | N | TYR | D | 220 | 23.468 | 6.498 | −1.072 | 1.00 | 33.94 | N |
| ATOM | 5348 | CA | TYR | D | 220 | 24.863 | 6.724 | −1.401 | 1.00 | 35.20 | C |
| ATOM | 5349 | CB | TYR | D | 220 | 25.328 | 5.817 | −2.543 | 1.00 | 35.67 | C |
| ATOM | 5350 | CG | TYR | D | 220 | 26.734 | 6.131 | −2.995 | 1.00 | 35.99 | C |
| ATOM | 5351 | CD1 | TYR | D | 220 | 27.834 | 5.657 | −2.289 | 1.00 | 35.60 | C |
| ATOM | 5352 | CE1 | TYR | D | 220 | 29.132 | 5.959 | −2.686 | 1.00 | 35.63 | C |
| ATOM | 5353 | CZ | TYR | D | 220 | 29.331 | 6.745 | −3.811 | 1.00 | 35.36 | C |
| ATOM | 5354 | OH | TYR | D | 220 | 30.611 | 7.033 | −4.208 | 1.00 | 35.50 | O |
| ATOM | 5355 | CE2 | TYR | D | 220 | 28.252 | 7.239 | −4.520 | 1.00 | 35.82 | C |
| ATOM | 5356 | CD2 | TYR | D | 220 | 26.962 | 6.926 | −4.115 | 1.00 | 34.81 | C |
| ATOM | 5357 | C | TYR | D | 220 | 25.003 | 8.171 | −1.843 | 1.00 | 38.14 | C |
| ATOM | 5358 | O | TYR | D | 220 | 24.359 | 8.597 | −2.807 | 1.00 | 38.27 | O |
| ATOM | 5359 | N | GLU | D | 221 | 25.839 | 8.925 | −1.136 | 1.00 | 40.89 | N |
| ATOM | 5360 | CA | GLU | D | 221 | 26.264 | 10.251 | −1.588 | 1.00 | 42.31 | C |
| ATOM | 5361 | CB | GLU | D | 221 | 26.091 | 11.302 | −0.492 | 1.00 | 44.07 | C |
| ATOM | 5362 | CG | GLU | D | 221 | 24.641 | 11.673 | −0.181 | 1.00 | 46.00 | C |
| ATOM | 5363 | CD | GLU | D | 221 | 23.901 | 12.367 | −1.334 | 1.00 | 48.48 | C |
| ATOM | 5364 | OE1 | GLU | D | 221 | 24.575 | 12.957 | −2.231 | 1.00 | 48.56 | O |
| ATOM | 5365 | OE2 | GLU | D | 221 | 22.648 | 12.270 | −1.380 | 1.00 | 48.86 | O |
| ATOM | 5366 | C | GLU | D | 221 | 27.734 | 10.114 | −1.990 | 1.00 | 43.02 | C |
| ATOM | 5367 | O | GLU | D | 221 | 28.496 | 9.418 | −1.303 | 1.00 | 44.02 | O |
| ATOM | 5368 | N | PRO | D | 222 | 28.137 | 10.742 | −3.119 | 1.00 | 42.60 | N |
| ATOM | 5369 | CA | PRO | D | 222 | 29.572 | 10.669 | −3.457 | 1.00 | 41.83 | C |
| ATOM | 5370 | CB | PRO | D | 222 | 29.647 | 11.190 | −4.897 | 1.00 | 42.60 | C |
| ATOM | 5371 | CG | PRO | D | 222 | 28.369 | 11.923 | −5.109 | 1.00 | 43.24 | C |
| ATOM | 5372 | CD | PRO | D | 222 | 27.327 | 11.323 | −4.202 | 1.00 | 42.15 | C |
| ATOM | 5373 | C | PRO | D | 222 | 30.429 | 11.473 | −2.490 | 1.00 | 41.18 | C |
| ATOM | 5374 | O | PRO | D | 222 | 29.891 | 12.327 | −1.783 | 1.00 | 40.76 | O |
| ATOM | 5375 | N | PRO | D | 223 | 31.757 | 11.227 | −2.449 | 1.00 | 41.47 | N |
| ATOM | 5376 | CA | PRO | D | 223 | 32.629 | 11.976 | −1.506 | 1.00 | 42.96 | C |
| ATOM | 5377 | CB | PRO | D | 223 | 34.024 | 11.489 | −1.837 | 1.00 | 42.70 | C |
| ATOM | 5378 | CG | PRO | D | 223 | 33.876 | 10.246 | −2.693 | 1.00 | 43.31 | C |
| ATOM | 5379 | CD | PRO | D | 223 | 32.476 | 10.203 | −3.249 | 1.00 | 42.22 | C |
| ATOM | 5380 | C | PRO | D | 223 | 32.511 | 13.511 | −1.713 | 1.00 | 44.93 | C |
| ATOM | 5381 | O | PRO | D | 223 | 32.301 | 13.998 | −2.837 | 1.00 | 43.16 | O |
| ATOM | 5382 | N | GLU | D | 224 | 32.620 | 14.274 | −0.625 | 1.00 | 48.00 | N |
| ATOM | 5383 | CA | GLU | D | 224 | 32.642 | 15.738 | −0.724 | 1.00 | 51.86 | C |
| ATOM | 5384 | CB | GLU | D | 224 | 32.471 | 16.403 | 0.650 | 1.00 | 53.19 | C |
| ATOM | 5385 | CG | GLU | D | 224 | 31.067 | 16.241 | 1.217 | 1.00 | 54.18 | C |
| ATOM | 5386 | CD | GLU | D | 224 | 30.802 | 17.192 | 2.376 | 1.00 | 55.42 | C |
| ATOM | 5387 | OE1 | GLU | D | 224 | 31.696 | 17.338 | 3.238 | 1.00 | 56.59 | O |
| ATOM | 5388 | OE2 | GLU | D | 224 | 29.705 | 17.793 | 2.432 | 1.00 | 55.35 | O |
| ATOM | 5389 | C | GLU | D | 224 | 33.942 | 16.178 | −1.391 | 1.00 | 52.44 | C |
| ATOM | 5390 | O | GLU | D | 224 | 34.882 | 15.389 | −1.520 | 1.00 | 52.31 | O |
| ATOM | 5391 | N | VAL | D | 225 | 34.032 | 17.458 | −1.723 | 1.00 | 50.96 | N |
| ATOM | 5392 | CA | VAL | D | 225 | 35.273 | 18.050 | −2.213 | 1.00 | 50.57 | C |
| ATOM | 5393 | CB | VAL | D | 225 | 35.052 | 19.502 | −2.713 | 1.00 | 50.39 | C |
| ATOM | 5394 | CG1 | VAL | D | 225 | 36.355 | 20.166 | −3.131 | 1.00 | 50.16 | C |
| ATOM | 5395 | CG2 | VAL | D | 225 | 34.098 | 19.490 | −3.911 | 1.00 | 50.84 | C |
| ATOM | 5396 | C | VAL | D | 225 | 36.323 | 17.960 | −1.084 | 1.00 | 49.48 | C |
| ATOM | 5397 | O | VAL | D | 225 | 36.055 | 18.328 | 0.067 | 1.00 | 50.18 | O |
| ATOM | 5398 | N | GLY | D | 226 | 37.486 | 17.417 | −1.425 | 1.00 | 47.65 | N |
| ATOM | 5399 | CA | GLY | D | 226 | 38.531 | 17.115 | −0.441 | 1.00 | 47.88 | C |
| ATOM | 5400 | C | GLY | D | 226 | 38.630 | 15.657 | −0.029 | 1.00 | 46.71 | C |
| ATOM | 5401 | O | GLY | D | 226 | 39.707 | 15.214 | 0.380 | 1.00 | 44.39 | O |
| ATOM | 5402 | N | SER | D | 227 | 37.526 | 14.912 | −0.119 | 1.00 | 47.48 | N |
| ATOM | 5403 | CA | SER | D | 227 | 37.495 | 13.502 | 0.303 | 1.00 | 47.11 | C |
| ATOM | 5404 | CB | SER | D | 227 | 36.193 | 13.206 | 1.033 | 1.00 | 48.36 | C |
| ATOM | 5405 | OG | SER | D | 227 | 36.200 | 11.889 | 1.592 | 1.00 | 51.02 | O |
| ATOM | 5406 | C | SER | D | 227 | 37.672 | 12.539 | −0.871 | 1.00 | 45.19 | C |
| ATOM | 5407 | O | SER | D | 227 | 37.338 | 12.887 | −2.006 | 1.00 | 43.79 | O |
| ATOM | 5408 | N | ASP | D | 228 | 38.208 | 11.361 | −0.577 | 1.00 | 44.65 | N |
| ATOM | 5409 | CA | ASP | D | 228 | 36.325 | 10.277 | −1.567 | 1.00 | 45.29 | C |
| ATOM | 5410 | CB | ASP | D | 228 | 39.774 | 9.740 | −1.654 | 1.00 | 47.77 | C |
| ATOM | 5411 | CG | ASP | D | 228 | 40.758 | 10.748 | −2.237 | 1.00 | 50.22 | C |
| ATOM | 5412 | OD1 | ASP | D | 228 | 40.325 | 11.689 | −2.953 | 1.00 | 49.85 | O |
| ATOM | 5413 | OD2 | ASP | D | 228 | 41.970 | 10.604 | −1.972 | 1.00 | 52.99 | O |
| ATOM | 5414 | C | ASP | D | 228 | 37.348 | 9.126 | −1.334 | 1.00 | 43.86 | C |
| ATOM | 5415 | O | ASP | D | 228 | 37.334 | 8.184 | −2.123 | 1.00 | 44.42 | O |
| ATOM | 5416 | N | CYS | D | 229 | 36.542 | 9.209 | −0.278 | 1.00 | 41.50 | N |
| ATOM | 5417 | CA | CYS | D | 229 | 35.589 | 8.102 | 0.064 | 1.00 | 39.45 | C |
| ATOM | 5418 | CB | CYS | D | 229 | 36.253 | 7.025 | 0.931 | 1.00 | 38.67 | C |
| ATOM | 5419 | SG | CYS | D | 229 | 36.892 | 7.669 | 2.499 | 1.00 | 35.12 | S |
| ATOM | 5420 | C | CYS | D | 229 | 34.374 | 8.656 | 0.787 | 1.00 | 38.54 | C |
| ATOM | 5421 | O | CYS | D | 229 | 34.401 | 9.798 | 1.269 | 1.00 | 38.02 | O |
| ATOM | 5422 | N | THR | D | 230 | 33.317 | 7.849 | 0.866 | 1.00 | 37.12 | N |
| ATOM | 5423 | CA | THR | D | 230 | 32.102 | 8.193 | 1.619 | 1.00 | 35.83 | C |
| ATOM | 5424 | CB | THR | D | 230 | 30.841 | 7.856 | 0.801 | 1.00 | 35.58 | C |
| ATOM | 5425 | OG1 | THR | D | 230 | 30.909 | 8.543 | −0.471 | 1.00 | 36.12 | O |
| ATOM | 5426 | CG2 | THR | D | 230 | 29.583 | 8.291 | 1.525 | 1.00 | 35.31 | C |
| ATOM | 5427 | C | THR | D | 230 | 32.070 | 7.407 | 2.924 | 1.00 | 33.29 | C |
| ATOM | 5428 | O | THR | D | 230 | 32.270 | 6.198 | 2.911 | 1.00 | 32.96 | O |
| ATOM | 5429 | N | THR | D | 231 | 31.807 | 8.090 | 4.034 | 1.00 | 31.80 | N |
| ATOM | 5430 | CA | THR | D | 231 | 31.844 | 7.498 | 5.374 | 1.00 | 30.84 | C |
| ATOM | 5431 | CB | THR | D | 231 | 32.760 | 8.313 | 6.316 | 1.00 | 30.64 | C |
| ATOM | 5432 | OG1 | THR | D | 231 | 34.077 | 8.382 | 5.752 | 1.00 | 31.46 | O |
| ATOM | 5433 | CG2 | THR | D | 231 | 32.842 | 7.676 | 7.710 | 1.00 | 30.15 | C |
| ATOM | 5434 | C | THR | D | 231 | 30.442 | 7.420 | 5.981 | 1.00 | 30.42 | C |
| ATOM | 5435 | O | THR | D | 231 | 29.757 | 8.440 | 6.102 | 1.00 | 28.62 | O |
| ATOM | 5436 | N | ILE | D | 232 | 30.026 | 6.210 | 6.354 | 1.00 | 30.06 | N |
| ATOM | 5437 | CA | ILE | D | 232 | 28.785 | 5.987 | 7.097 | 1.00 | 30.65 | C |
| ATOM | 5438 | CB | ILE | D | 232 | 27.948 | 4.855 | 6.447 | 1.00 | 30.39 | C |
| ATOM | 5439 | CG1 | ILE | D | 232 | 27.496 | 5.270 | 5.042 | 1.00 | 31.39 | C |
| ATOM | 5440 | CD1 | ILE | D | 232 | 26.968 | 4.130 | 4.207 | 1.00 | 31.63 | C |
| ATOM | 5441 | CG2 | ILE | D | 232 | 26.728 | 4.515 | 7.299 | 1.00 | 30.45 | C |
| ATOM | 5442 | C | ILE | D | 232 | 29.136 | 5.630 | 8.538 | 1.00 | 31.15 | C |
| ATOM | 5443 | O | ILE | D | 232 | 30.102 | 4.906 | 8.776 | 1.00 | 32.32 | O |
| ATOM | 5444 | N | HIS | D | 233 | 28.349 | 6.131 | 9.489 | 1.00 | 31.78 | N |
| ATOM | 5445 | CA | HIS | D | 233 | 28.539 | 5.837 | 10.910 | 1.00 | 32.77 | C |
| ATOM | 5446 | CB | HIS | D | 233 | 28.414 | 7.111 | 11.752 | 1.00 | 34.34 | C |
| ATOM | 5447 | CG | HIS | D | 233 | 29.567 | 8.052 | 11.611. | 1.00 | 36.58 | C |
| ATOM | 5448 | ND1 | HIS | D | 233 | 29.494 | 9.375 | 11.998 | 1.00 | 39.46 | N |
| ATOM | 5449 | CE1 | HIS | D | 233 | 30.652 | 9.966 | 11.761 | 1.00 | 39.62 | C |
| ATOM | 5450 | NE2 | HIS | D | 233 | 31.476 | 9.072 | 11.235 | 1.00 | 39.24 | N |
| ATOM | 5451 | CD2 | HIS | D | 233 | 30.821 | 7.867 | 11.128 | 1.00 | 37.96 | C |
| ATOM | 5452 | C | HIS | D | 233 | 27.518 | 4.813 | 11.385 | 1.00 | 32.46 | C |
| ATOM | 5453 | O | HIS | D | 233 | 26.355 | 5.155 | 11.600 | 1.00 | 32.07 | O |
| ATOM | 5454 | N | TYR | D | 234 | 27.964 | 3.571 | 11.555 | 1.00 | 31.69 | N |
| ATOM | 5455 | CA | TYR | D | 234 | 27.124 | 2.502 | 12.092 | 1.00 | 31.16 | C |
| ATOM | 5456 | CB | TYR | D | 234 | 27.490 | 1.152 | 11.459 | 1.00 | 31.04 | C |
| ATOM | 5457 | CG | TYR | D | 234 | 27.162 | 1.051 | 9.984 | 1.00 | 31.82 | C |
| ATOM | 5458 | CD1 | TYR | D | 234 | 25.836 | 0.957 | 9.544 | 1.00 | 31.72 | C |
| ATOM | 5459 | CE1 | TYR | D | 234 | 25.532 | 0.873 | 8.190 | 1.00 | 32.03 | C |
| ATOM | 5460 | CZ | TYR | D | 234 | 26.558 | 0.877 | 7.257 | 1.00 | 32.12 | C |
| ATOM | 5461 | OH | TYR | D | 234 | 26.265 | 0.773 | 5.917 | 1.00 | 33.07 | O |
| ATOM | 5462 | CE2 | TYR | D | 234 | 27.873 | 0.970 | 7.666 | 1.00 | 32.41 | C |
| ATOM | 5463 | CD2 | TYR | D | 234 | 28.175 | 1.057 | 9.016 | 1.00 | 31.79 | C |
| ATOM | 5464 | C | TYR | D | 234 | 27.242 | 2.417 | 13.617 | 1.00 | 31.64 | C |
| ATOM | 5465 | O | TYR | D | 234 | 28.251 | 2.821 | 14.192 | 1.00 | 31.67 | O |
| ATOM | 5466 | N | ASN | D | 235 | 26.197 | 1.906 | 14.261 | 1.00 | 32.01 | N |
| ATOM | 5467 | CA | ASN | D | 235 | 26.216 | 1.561 | 15.686 | 1.00 | 33.02 | C |
| ATOM | 5468 | CB | ASN | D | 235 | 25.270 | 2.449 | 16.507 | 1.00 | 33.59 | C |
| ATOM | 5469 | CG | ASN | D | 235 | 25.519 | 3.932 | 16.321 | 1.00 | 35.09 | C |
| ATOM | 5470 | OD1 | ASN | D | 235 | 24.576 | 4.713 | 16.351 | 1.00 | 37.49 | O |
| ATOM | 5471 | ND2 | ASN | D | 235 | 26.782 | 4.329 | 16.146 | 1.00 | 35.41 | N |
| ATOM | 5472 | C | ASN | D | 235 | 25.758 | 0.120 | 15.837 | 1.00 | 32.75 | C |
| ATOM | 5473 | O | ASN | D | 235 | 24.791 | −0.279 | 15.192 | 1.00 | 31.48 | O |
| ATOM | 5474 | N | TYR | D | 236 | 26.444 | −0.641 | 16.686 | 1.00 | 33.92 | N |
| ATOM | 5475 | CA | TYR | D | 236 | 26.052 | −2.010 | 17.008 | 1.00 | 35.21 | C |
| ATOM | 5476 | CB | TYR | D | 236 | 27.256 | −2.943 | 16.885 | 1.00 | 34.04 | C |
| ATOM | 5477 | CG | TYR | D | 236 | 27.729 | −3.092 | 15.453 | 1.00 | 33.68 | C |
| ATOM | 5478 | CD1 | TYR | D | 236 | 27.198 | −4.088 | 14.629 | 1.00 | 34.21 | C |
| ATOM | 5479 | CE1 | TYR | D | 236 | 27.622 | −4.236 | 13.316 | 1.00 | 33.67 | C |
| ATOM | 5480 | CZ | TYR | D | 236 | 28.589 | −3.375 | 12.805 | 1.00 | 34.57 | C |
| ATOM | 5481 | OH | TYR | D | 236 | 29.002 | −3.497 | 11.489 | 1.00 | 35.96 | O |
| ATOM | 5482 | CE2 | TYR | D | 236 | 29.113 | −2.366 | 13.595 | 1.00 | 34.55 | C |
| ATOM | 5483 | CD2 | TYR | D | 236 | 28.703 | −2.240 | 14.915 | 1.00 | 33.38 | C |
| ATOM | 5484 | C | TYR | D | 236 | 25.458 | −2.029 | 18.417 | 1.00 | 37.11 | C |
| ATOM | 5485 | O | TYR | D | 236 | 26.120 | −1.649 | 19.387 | 1.00 | 37.03 | O |
| ATOM | 5486 | N | MET | D | 237 | 24.214 | −2.505 | 18.510 | 1.00 | 41.06 | N |
| ATOM | 5487 | CA | MET | D | 237 | 23.375 | −2.345 | 19.699 | 1.00 | 46.77 | C |
| ATOM | 5488 | CB | MET | D | 237 | 22.012 | −1.799 | 19.265 | 1.00 | 47.99 | C |
| ATOM | 5489 | CG | MET | D | 237 | 22.054 | −0.528 | 18.404 | 1.00 | 49.04 | C |
| ATOM | 5490 | SD | MET | D | 237 | 22.882 | 0.904 | 19.158 | 1.00 | 51.46 | S |
| ATOM | 5491 | CE | MET | D | 237 | 21.950 | 1.065 | 20.670 | 1.00 | 50.57 | C |
| ATOM | 5492 | C | MET | D | 237 | 23.174 | −3.659 | 20.501 | 1.00 | 52.09 | C |
| ATOM | 5493 | O | MET | D | 237 | 22.236 | −3.776 | 21.282 | 1.00 | 55.09 | O |
| ATOM | 5494 | N | CYS | D | 238 | 24.064 | −4.629 | 20.328 | 1.00 | 56.55 | N |
| ATOM | 5495 | CA | CYS | D | 238 | 24.150 | −5.813 | 21.174 | 1.00 | 60.89 | C |
| ATOM | 5496 | CB | CYS | D | 238 | 23.367 | −6.941 | 20.547 | 1.00 | 64.18 | C |
| ATOM | 5497 | SG | CYS | D | 238 | 23.231 | −8.538 | 21.387 | 1.00 | 71.25 | S |
| ATOM | 5498 | C | CYS | D | 238 | 25.623 | −6.199 | 21.303 | 1.00 | 62.50 | C |
| ATOM | 5499 | O | CYS | D | 238 | 26.324 | −6.033 | 20.330 | 1.00 | 58.61 | O |
| ATOM | 5500 | N | ASN | D | 239 | 26.008 | −6.783 | 22.447 | 1.00 | 66.24 | N |
| ATOM | 5501 | CA | ASN | D | 239 | 27.349 | −7.225 | 22.707 | 1.00 | 68.90 | C |
| ATOM | 5502 | CB | ASN | D | 239 | 27.548 | −7.136 | 24.207 | 1.00 | 72.37 | C |
| ATOM | 5503 | CG | ASN | D | 239 | 29.009 | −7.290 | 24.640 | 1.00 | 74.78 | C |
| ATOM | 5504 | OD1 | ASN | D | 239 | 29.482 | −8.396 | 24.965 | 1.00 | 80.51 | O |
| ATOM | 5505 | ND2 | ASN | D | 239 | 29.717 | −6.155 | 24.738 | 1.00 | 74.85 | N |
| ATOM | 5506 | C | ASN | D | 239 | 27.535 | −8.676 | 22.276 | 1.00 | 67.32 | C |
| ATOM | 5507 | O | ASN | D | 239 | 26.561 | −9.406 | 22.198 | 1.00 | 68.07 | O |
| ATOM | 5508 | N | SER | D | 240 | 28.770 | −9.032 | 21.948 | 1.00 | 65.37 | N |
| ATOM | 5509 | CA | SER | D | 240 | 29.075 | −10.375 | 21.458 | 1.00 | 64.94 | C |
| ATOM | 5510 | CB | SER | D | 240 | 30.466 | −10.428 | 20.806 | 1.00 | 62.55 | C |
| ATOM | 5511 | OG | SER | D | 240 | 30.506 | −9.612 | 19.642 | 1.00 | 57.31 | O |
| ATOM | 5512 | C | SER | D | 240 | 28.959 | −11.428 | 22.560 | 1.00 | 66.15 | C |
| ATOM | 5513 | O | SER | D | 240 | 28.575 | −12.573 | 22.282 | 1.00 | 65.30 | O |
| ATOM | 5514 | N | SER | D | 241 | 29.241 | −11.043 | 23.812 | 1.00 | 66.16 | N |
| ATOM | 5515 | CA | SER | D | 241 | 28.918 | −11.904 | 24.979 | 1.00 | 67.09 | C |
| ATOM | 5516 | CB | SER | D | 241 | 29.664 | −11.437 | 26.231 | 1.00 | 66.86 | C |
| ATOM | 5517 | OG | SER | D | 241 | 29.189 | −10.180 | 26.691 | 1.00 | 66.52 | O |
| ATOM | 5518 | C | SER | D | 241 | 27.404 | −11.958 | 25.253 | 1.00 | 66.52 | C |
| ATOM | 5519 | O | SER | D | 241 | 26.933 | −12.783 | 26.028 | 1.00 | 66.29 | O |
| ATOM | 5520 | N | SER | D | 249 | 28.231 | −17.540 | 18.211 | 1.00 | 70.55 | N |
| ATOM | 5521 | CA | SER | D | 249 | 28.813 | −17.454 | 16.865 | 1.00 | 70.56 | C |
| ATOM | 5522 | CB | SER | D | 249 | 27.805 | −17.922 | 15.802 | 1.00 | 70.26 | C |
| ATOM | 5523 | OG | SER | D | 249 | 28.387 | −17.920 | 14.496 | 1.00 | 70.05 | O |
| ATOM | 5524 | C | SER | D | 249 | 29.256 | −16.009 | 16.547 | 1.00 | 68.69 | C |
| ATOM | 5525 | O | SER | D | 249 | 28.595 | −15.061 | 16.979 | 1.00 | 69.51 | O |
| ATOM | 5526 | N | PRO | D | 250 | 30.392 | −15.859 | 15.841 | 1.00 | 67.18 | N |
| ATOM | 5527 | CA | PRO | D | 250 | 30.786 | −14.529 | 15.363 | 1.00 | 65.07 | C |
| ATOM | 5528 | CB | PRO | D | 250 | 32.125 | −14.788 | 14.651 | 1.00 | 67.00 | C |
| ATOM | 5529 | CG | PRO | D | 250 | 32.638 | −16.069 | 15.213 | 1.00 | 68.06 | C |
| ATOM | 5530 | CD | PRO | D | 250 | 31.425 | −16.876 | 15.549 | 1.00 | 67.85 | C |
| ATOM | 5531 | C | PRO | D | 250 | 29.765 | −13.935 | 14.389 | 1.00 | 60.61 | C |
| ATOM | 5532 | O | PRO | D | 250 | 29.116 | −14.686 | 13.649 | 1.00 | 57.88 | O |
| ATOM | 5533 | N | ILE | D | 251 | 29.657 | −12.611 | 14.375 | 1.00 | 56.40 | N |
| ATOM | 5534 | CA | ILE | D | 251 | 28.736 | −11.911 | 13.478 | 1.00 | 54.83 | C |
| ATOM | 5535 | CB | ILE | D | 251 | 27.992 | −10.788 | 14.231 | 1.00 | 55.89 | C |
| ATOM | 5536 | CG1 | ILE | D | 251 | 27.097 | −11.368 | 15.329 | 1.00 | 57.22 | C |
| ATOM | 5537 | CD1 | ILE | D | 251 | 26.846 | −10.395 | 16.466 | 1.00 | 57.05 | C |
| ATOM | 5538 | CG2 | ILE | D | 251 | 27.188 | −9.910 | 13.270 | 1.00 | 57.04 | C |
| ATOM | 5539 | C | ILE | D | 251 | 29.523 | −11.339 | 12.288 | 1.00 | 50.25 | C |
| ATOM | 5540 | O | ILE | D | 251 | 30.666 | −10.894 | 12.445 | 1.00 | 48.04 | O |
| ATOM | 5541 | N | LEU | D | 252 | 28.907 | −11.403 | 11.105 | 1.00 | 45.27 | N |
| ATOM | 5542 | CA | LEU | D | 252 | 29.447 | −10.831 | 9.883 | 1.00 | 42.37 | C |
| ATOM | 5543 | CB | LEU | D | 252 | 29.511 | −11.898 | 8.785 | 1.00 | 45.38 | C |
| ATOM | 5544 | CG | LEU | D | 252 | 30.701 | −12.856 | 8.919 | 1.00 | 47.83 | C |
| ATOM | 5545 | CD1 | LEU | D | 252 | 30.438 | −14.118 | 8.089 | 1.00 | 48.64 | C |
| ATOM | 5546 | CD2 | LEU | D | 252 | 31.992 | −12.165 | 8.465 | 1.00 | 47.87 | C |
| ATOM | 5547 | C | LEU | D | 252 | 28.520 | −9.725 | 9.437 | 1.00 | 36.94 | C |
| ATOM | 5548 | O | LEU | D | 252 | 27.315 | −9.934 | 9.382 | 1.00 | 33.84 | O |
| ATOM | 5549 | N | THR | D | 253 | 29.086 | −8.559 | 9.117 | 1.00 | 33.27 | N |
| ATOM | 5550 | CA | THR | D | 253 | 28.328 | −7.459 | 8.529 | 1.00 | 30.44 | C |
| ATOM | 5551 | CB | THR | D | 253 | 28.776 | −6.101 | 9.102 | 1.00 | 30.13 | C |
| ATOM | 5552 | OG1 | THR | D | 253 | 28.556 | −6.094 | 10.516 | 1.00 | 29.93 | O |
| ATOM | 5553 | CG2 | THR | D | 253 | 28.018 | −4.944 | 8.454 | 1.00 | 29.22 | C |
| ATOM | 5554 | C | THR | D | 253 | 28.530 | −7.484 | 7.015 | 1.00 | 29.16 | C |
| ATOM | 5555 | O | THR | D | 253 | 29.667 | −7.546 | 6.543 | 1.00 | 26.91 | O |
| ATOM | 5556 | N | ILE | D | 254 | 27.423 | −7.434 | 6.271 | 1.00 | 28.47 | N |
| ATOM | 5557 | CA | ILE | D | 254 | 27.441 | −7.428 | 4.811 | 1.00 | 27.84 | C |
| ATOM | 5558 | CB | ILE | D | 254 | 26.506 | −8.492 | 4.223 | 1.00 | 27.42 | C |
| ATOM | 5559 | CG1 | ILE | D | 254 | 26.976 | −9.895 | 4.616 | 1.00 | 27.69 | C |
| ATOM | 5560 | CD1 | ILE | D | 254 | 25.886 | −10.953 | 4.414 | 1.00 | 27.63 | C |
| ATOM | 5561 | CG2 | ILE | D | 254 | 26.415 | −8.352 | 2.694 | 1.00 | 27.66 | C |
| ATOM | 5562 | C | ILE | D | 254 | 26.981 | −6.055 | 4.339 | 1.00 | 27.80 | C |
| ATOM | 5563 | O | ILE | D | 254 | 25.848 | −5.666 | 4.605 | 1.00 | 26.74 | O |
| ATOM | 5564 | N | ILE | D | 255 | 27.864 | −5.346 | 3.631 | 1.00 | 27.94 | N |
| ATOM | 5565 | CA | ILE | D | 255 | 27.557 | −4.054 | 3.031 | 1.00 | 27.63 | C |
| ATOM | 5566 | CB | ILE | D | 255 | 28.694 | −3.026 | 3.253 | 1.00 | 27.42 | C |
| ATOM | 5567 | CG1 | ILE | D | 255 | 29.068 | −2.915 | 4.742 | 1.00 | 26.44 | C |
| ATOM | 5568 | CD1 | ILE | D | 255 | 27.938 | −2.518 | 5.669 | 1.00 | 26.11 | C |
| ATOM | 5569 | CG2 | ILE | D | 255 | 28.300 | −1.654 | 2.709 | 1.00 | 27.57 | C |
| ATOM | 5570 | C | ILE | D | 255 | 27.360 | −4.279 | 1.540 | 1.00 | 28.94 | C |
| ATOM | 5571 | O | ILE | D | 255 | 28.259 | −4.784 | 0.870 | 1.00 | 29.71 | O |
| ATOM | 5572 | N | THR | D | 256 | 26.185 | −3.919 | 1.028 | 1.00 | 29.17 | N |
| ATOM | 5573 | CA | THR | D | 256 | 25.890 | −4.014 | −0.397 | 1.00 | 29.14 | C |
| ATOM | 5574 | CB | THR | D | 256 | 24.692 | −4.942 | −0.683 | 1.00 | 29.18 | C |
| ATOM | 5575 | OG1 | THR | D | 256 | 23.578 | −4.562 | 0.123 | 1.00 | 30.24 | O |
| ATOM | 5576 | CG2 | THR | D | 256 | 25.026 | −6.386 | −0.378 | 1.00 | 28.84 | C |
| ATOM | 5577 | C | THR | D | 256 | 25.599 | −2.641 | −0.989 | 1.00 | 30.21 | C |
| ATOM | 5578 | O | THR | D | 256 | 25.027 | −1.772 | −0.328 | 1.00 | 30.94 | O |
| ATOM | 5579 | N | LEU | D | 257 | 26.016 | −2.463 | −2.236 | 1.00 | 30.85 | N |
| ATOM | 5580 | CA | LEU | D | 257 | 25.670 | −1.295 | −3.026 | 1.00 | 31.60 | C |
| ATOM | 5581 | CB | LEU | D | 257 | 26.903 | −0.852 | −3.819 | 1.00 | 32.69 | C |
| ATOM | 5582 | CG | LEU | D | 257 | 27.002 | 0.622 | −4.227 | 1.00 | 34.18 | C |
| ATOM | 5583 | CD1 | LEU | D | 257 | 27.092 | 1.543 | −3.019 | 1.00 | 34.10 | C |
| ATOM | 5584 | CD2 | LEU | D | 257 | 28.197 | 0.821 | −5.148 | 1.00 | 35.20 | C |
| ATOM | 5585 | C | LEU | D | 257 | 24.514 | −1.707 | −3.955 | 1.00 | 31.02 | C |
| ATOM | 5586 | O | LEU | D | 257 | 24.632 | −2.705 | −4.667 | 1.00 | 30.75 | O |
| ATOM | 5587 | N | GLU | D | 258 | 23.409 | −0.962 | −3.916 | 1.00 | 30.82 | N |
| ATOM | 5588 | CA | GLU | D | 258 | 22.230 | −1.199 | −4.778 | 1.00 | 30.58 | C |
| ATOM | 5589 | CB | GLU | D | 258 | 20.977 | −1.494 | −3.939 | 1.00 | 30.90 | C |
| ATOM | 5590 | CG | GLU | D | 258 | 21.140 | −2.489 | −2.805 | 1.00 | 31.69 | C |
| ATOM | 5591 | CD | GLU | D | 258 | 19.862 | −2.705 | −2.011 | 1.00 | 31.89 | C |
| ATOM | 5592 | OE1 | GLU | D | 258 | 19.028 | −1.770 | −1.910 | 1.00 | 30.88 | O |
| ATOM | 5593 | OE2 | GLU | D | 258 | 19.707 | −3.824 | −1.474 | 1.00 | 31.08 | O |
| ATOM | 5594 | C | GLU | D | 258 | 21.894 | 0.038 | −5.595 | 1.00 | 30.50 | C |
| ATOM | 5595 | O | GLU | D | 258 | 22.148 | 1.157 | −5.140 | 1.00 | 30.09 | O |
| ATOM | 5596 | N | ASP | D | 259 | 21.261 | −0.149 | −6.755 | 1.00 | 30.14 | N |
| ATOM | 5597 | CA | ASP | D | 259 | 20.691 | 0.993 | −7.507 | 1.00 | 30.12 | C |
| ATOM | 5598 | CB | ASP | D | 259 | 20.642 | 0.734 | −9.032 | 1.00 | 30.25 | C |
| ATOM | 5599 | CG | ASP | D | 259 | 19.627 | −0.328 | −9.453 | 1.00 | 30.74 | C |
| ATOM | 5600 | OD1 | ASP | D | 259 | 18.723 | −0.712 | −8.678 | 1.00 | 31.06 | O |
| ATOM | 5601 | OD2 | ASP | D | 259 | 19.725 | −0.775 | −10.616 | 1.00 | 31.40 | O |
| ATOM | 5602 | C | ASP | D | 259 | 19.326 | 1.388 | −6.924 | 1.00 | 30.48 | C |
| ATOM | 5603 | O | ASP | D | 259 | 18.856 | 0.762 | −5.965 | 1.00 | 29.72 | O |
| ATOM | 5604 | N | SER | D | 260 | 18.698 | 2.411 | −7.503 | 1.00 | 30.13 | N |
| ATOM | 5605 | CA | SER | D | 260 | 17.389 | 2.876 | −7.041 | 1.00 | 30.22 | C |
| ATOM | 5606 | CB | SER | D | 260 | 16.971 | 4.158 | −7.781 | 1.00 | 30.81 | C |
| ATOM | 5607 | OG | SER | D | 260 | 17.113 | 4.013 | −9.175 | 1.00 | 34.44 | O |
| ATOM | 5608 | C | SER | D | 260 | 16.287 | 1.831 | −7.141 | 1.00 | 29.44 | C |
| ATOM | 5609 | O | SER | D | 260 | 15.362 | 1.844 | −6.333 | 1.00 | 28.50 | O |
| ATOM | 5610 | N | SER | D | 261 | 16.393 | 0.921 | −8.111 | 1.00 | 29.59 | N |
| ATOM | 5611 | CA | SER | D | 261 | 15.414 | −0.160 | −8.306 | 1.00 | 28.71 | C |
| ATOM | 5612 | CB | SER | D | 261 | 15.278 | −0.477 | −9.799 | 1.00 | 28.91 | C |
| ATOM | 5613 | OG | SER | D | 261 | 15.039 | 0.699 | −10.557 | 1.00 | 28.76 | O |
| ATOM | 5614 | C | SER | D | 261 | 15.749 | −1.442 | −7.543 | 1.00 | 28.84 | C |
| ATOM | 5615 | O | SER | D | 261 | 15.072 | −2.454 | −7.735 | 1.00 | 30.41 | O |
| ATOM | 5616 | N | GLY | D | 262 | 16.778 | −1.418 | −6.690 | 1.00 | 29.20 | N |
| ATOM | 5617 | CA | GLY | D | 262 | 17.116 | −2.563 | −5.842 | 1.00 | 28.52 | C |
| ATOM | 5618 | C | GLY | D | 262 | 18.031 | −3.626 | −6.436 | 1.00 | 28.76 | C |
| ATOM | 5619 | O | GLY | D | 262 | 18.228 | −4.672 | −5.812 | 1.00 | 28.61 | O |
| ATOM | 5620 | N | ASN | D | 263 | 18.601 | −3.378 | −7.624 | 1.00 | 27.97 | N |
| ATOM | 5621 | CA | ASN | D | 263 | 19.563 | −4.311 | −8.229 | 1.00 | 27.25 | C |
| ATOM | 5622 | CB | ASN | D | 263 | 19.749 | −4.044 | −9.722 | 1.00 | 27.17 | C |
| ATOM | 5623 | CG | ASN | D | 263 | 18.458 | −4.196 | −10.512 | 1.00 | 27.36 | C |
| ATOM | 5624 | OD1 | ASN | D | 263 | 17.957 | −5.306 | −10.663 | 1.00 | 26.82 | O |
| ATOM | 5625 | ND2 | ASN | D | 263 | 17.919 | −3.079 | −11.027 | 1.00 | 26.57 | N |
| ATOM | 5626 | C | ASN | D | 263 | 20.915 | −4.210 | −7.531 | 1.00 | 27.34 | C |
| ATOM | 5627 | O | ASN | D | 263 | 21.387 | −3.119 | −7.231 | 1.00 | 26.21 | O |
| ATOM | 5628 | N | LEU | D | 264 | 21.545 | −5.361 | −7.296 | 1.00 | 27.82 | N |
| ATOM | 5629 | CA | LEU | D | 264 | 22.857 | −5.413 | −6.652 | 1.00 | 27.80 | C |
| ATOM | 5630 | CB | LEU | D | 264 | 23.129 | −6.812 | −6.086 | 1.00 | 28.93 | C |
| ATOM | 5631 | CG | LEU | D | 264 | 22.688 | −7.068 | −4.608 | 1.00 | 30.75 | C |
| ATOM | 5632 | CD1 | LEU | D | 264 | 21.294 | −6.589 | −4.159 | 1.00 | 30.70 | C |
| ATOM | 5633 | CD2 | LEU | D | 264 | 22.767 | −8.553 | −4.303 | 1.00 | 31.65 | C |
| ATOM | 5634 | C | LEU | D | 264 | 23.967 | −4.920 | −7.585 | 1.00 | 27.12 | C |
| ATOM | 5635 | O | LEU | D | 264 | 24.079 | −5.366 | −8.720 | 1.00 | 26.87 | O |
| ATOM | 5636 | N | LEU | D | 265 | 24.754 | −3.967 | −7.094 | 1.00 | 26.29 | N |
| ATOM | 5637 | CA | LEU | D | 265 | 25.917 | −3.435 | −7.802 | 1.00 | 25.90 | C |
| ATOM | 5638 | CB | LEU | D | 265 | 25.899 | −1.904 | −7.742 | 1.00 | 25.21 | C |
| ATOM | 5639 | CG | LEU | D | 265 | 24.599 | −1.222 | −8.170 | 1.00 | 25.05 | C |
| ATOM | 5640 | CD1 | LEU | D | 265 | 24.756 | 0.292 | −8.078 | 1.00 | 24.84 | C |
| ATOM | 5641 | CD2 | LEU | D | 265 | 24.182 | −1.668 | −9.570 | 1.00 | 24.92 | C |
| ATOM | 5642 | C | LEU | D | 265 | 27.244 | −3.955 | −7.249 | 1.00 | 26.61 | C |
| ATOM | 5643 | O | LEU | D | 265 | 28.208 | −4.121 | −8.009 | 1.00 | 25.94 | O |
| ATOM | 5644 | N | GLY | D | 266 | 27.296 | −4.215 | −5.943 | 1.00 | 26.85 | N |
| ATOM | 5645 | CA | GLY | D | 266 | 28.541 | −4.663 | −5.310 | 1.00 | 26.97 | C |
| ATOM | 5646 | C | GLY | D | 266 | 28.324 | −5.109 | −3.883 | 1.00 | 27.34 | C |
| ATOM | 5647 | O | GLY | D | 266 | 27.302 | −4.775 | −3.265 | 1.00 | 26.70 | O |
| ATOM | 5648 | N | ARG | D | 267 | 29.291 | −5.863 | −3.365 | 1.00 | 28.33 | N |
| ATOM | 5649 | CA | ARG | D | 267 | 29.193 | −6.443 | −2.021 | 1.00 | 29.29 | C |
| ATOM | 5650 | CB | ARG | D | 267 | 28.457 | −7.785 | −2.057 | 1.00 | 28.76 | C |
| ATOM | 5651 | CG | ARG | D | 267 | 28.109 | −8.337 | −0.687 | 1.00 | 28.90 | C |
| ATOM | 5652 | CD | ARG | D | 267 | 27.586 | −9.787 | −0.698 | 1.00 | 28.76 | C |
| ATOM | 5653 | NE | ARG | D | 267 | 26.332 | −9.983 | −1.417 | 1.00 | 29.05 | N |
| ATOM | 5654 | CZ | ARG | D | 267 | 26.179 | −10.545 | −2.619 | 1.00 | 28.69 | C |
| ATOM | 5655 | NH1 | ARG | D | 267 | 27.212 | −11.010 | −3.325 | 1.00 | 28.96 | N |
| ATOM | 5656 | NH2 | ARG | D | 267 | 24.957 | −10.642 | −3.137 | 1.00 | 28.03 | N |
| ATOM | 5657 | C | ARG | D | 267 | 30.575 | −6.621 | −1.393 | 1.00 | 29.71 | C |
| ATOM | 5658 | O | ARG | D | 267 | 31.529 | −7.010 | −2.059 | 1.00 | 30.17 | O |
| ATOM | 5659 | N | ASN | D | 268 | 30.656 | −6.337 | −0.102 | 1.00 | 30.08 | N |
| ATOM | 5660 | CA | ASN | D | 268 | 31.823 | −6.645 | 0.725 | 1.00 | 30.52 | C |
| ATOM | 5661 | CB | ASN | D | 268 | 32.762 | −5.452 | 0.801 | 1.00 | 31.80 | C |
| ATOM | 5662 | CG | ASN | D | 268 | 33.719 | −5.414 | −0.361 | 1.00 | 34.27 | C |
| ATOM | 5663 | OD1 | ASN | D | 268 | 34.622 | −6.251 | −0.453 | 1.00 | 36.66 | O |
| ATOM | 5664 | ND2 | ASN | D | 268 | 33.530 | −4.440 | −1.263 | 1.00 | 34.82 | N |
| ATOM | 5665 | C | ASN | D | 268 | 31.326 | −7.023 | 2.112 | 1.00 | 29.70 | C |
| ATOM | 5666 | O | ASN | D | 268 | 30.127 | −6.932 | 2.398 | 1.00 | 28.37 | O |
| ATOM | 5667 | N | SER | D | 269 | 32.241 | −7.465 | 2.961 | 1.00 | 29.01 | N |
| ATOM | 5668 | CA | SER | D | 269 | 31.871 | −7.885 | 4.296 | 1.00 | 28.39 | C |
| ATOM | 5669 | CB | SER | D | 269 | 31.236 | −9.279 | 4.261 | 1.00 | 28.63 | C |
| ATOM | 5670 | OG | SER | D | 269 | 32.142 | −10.228 | 3.745 | 1.00 | 29.12 | O |
| ATOM | 5671 | C | SER | D | 269 | 33.045 | −7.888 | 5.256 | 1.00 | 27.48 | C |
| ATOM | 5672 | O | SER | D | 269 | 34.210 | −7.916 | 4.849 | 1.00 | 26.85 | O |
| ATOM | 5673 | N | PHE | D | 270 | 32.712 | −7.869 | 6.539 | 1.00 | 28.24 | N |
| ATOM | 5674 | CA | PHE | D | 270 | 33.700 | −7.936 | 7.600 | 1.00 | 28.44 | C |
| ATOM | 5675 | CB | PHE | D | 270 | 34.309 | −6.558 | 7.875 | 1.00 | 28.25 | C |
| ATOM | 5676 | CG | PHE | D | 270 | 33.317 | −5.485 | 8.235 | 1.00 | 28.49 | C |
| ATOM | 5677 | CD1 | PHE | D | 270 | 32.645 | −4.776 | 7.244 | 1.00 | 28.86 | C |
| ATOM | 5678 | CE1 | PHE | D | 270 | 31.754 | −3.755 | 7.573 | 1.00 | 28.07 | C |
| ATOM | 5679 | CZ | PHE | D | 270 | 31.534 | −3.431 | 8.909 | 1.00 | 28.29 | C |
| ATOM | 5680 | CE2 | PHE | D | 270 | 32.206 | −4.122 | 9.905 | 1.00 | 28.46 | C |
| ATOM | 5681 | CD2 | PHE | D | 270 | 33.095 | −5.140 | 9.569 | 1.00 | 28.83 | C |
| ATOM | 5682 | C | PHE | D | 270 | 33.084 | −8.522 | 8.863 | 1.00 | 29.39 | C |
| ATOM | 5683 | O | PHE | D | 270 | 31.874 | −8.404 | 9.107 | 1.00 | 26.58 | O |
| ATOM | 5684 | N | GLU | D | 271 | 33.943 | −9.162 | 9.646 | 1.00 | 31.75 | N |
| ATOM | 5685 | CA | GLU | D | 271 | 33.549 | −9.729 | 10.926 | 1.00 | 33.12 | C |
| ATOM | 5686 | CB | GLU | D | 271 | 34.580 | −10.760 | 11.356 | 1.00 | 34.53 | C |
| ATOM | 5687 | CG | GLU | D | 271 | 34.084 | −11.806 | 12.347 | 1.00 | 35.94 | C |
| ATOM | 5688 | CD | GLU | D | 271 | 35.062 | −12.995 | 12.442 | 1.00 | 37.20 | C |
| ATOM | 5689 | OE1 | GLU | D | 271 | 35.486 | −13.484 | 11.362 | 1.00 | 35.94 | O |
| ATOM | 5690 | OE2 | GLU | D | 271 | 35.379 | −13.437 | 13.589 | 1.00 | 38.45 | O |
| ATOM | 5691 | C | GLU | D | 271 | 33.450 | −8.598 | 11.929 | 1.00 | 33.09 | C |
| ATOM | 5692 | O | GLU | D | 271 | 34.157 | −7.584 | 11.803 | 1.00 | 32.56 | O |
| ATOM | 5693 | N | VAL | D | 272 | 32.544 | −8.732 | 12.894 | 1.00 | 33.47 | N |
| ATOM | 5694 | CA | VAL | D | 272 | 32.383 | −7.703 | 13.932 | 1.00 | 34.50 | C |
| ATOM | 5695 | CB | VAL | D | 272 | 31.178 | −6.764 | 13.654 | 1.00 | 35.20 | C |
| ATOM | 5696 | CG1 | VAL | D | 272 | 29.901 | −7.566 | 13.507 | 1.00 | 36.28 | C |
| ATOM | 5697 | CG2 | VAL | D | 272 | 31.043 | −5.690 | 14.735 | 1.00 | 34.96 | C |
| ATOM | 5698 | C | VAL | D | 272 | 32.294 | −8.351 | 15.310 | 1.00 | 34.24 | C |
| ATOM | 5699 | O | VAL | D | 272 | 31.684 | −9.413 | 15.481 | 1.00 | 33.06 | O |
| ATOM | 5700 | N | ARG | D | 273 | 32.969 | −7.722 | 16.276 | 1.00 | 36.06 | N |
| ATOM | 5701 | CA | ARG | D | 273 | 32.929 | −8.113 | 17.677 | 1.00 | 35.79 | C |
| ATOM | 5702 | CB | ARG | D | 273 | 34.248 | −8.751 | 18.100 | 1.00 | 37.06 | C |
| ATOM | 5703 | CG | ARG | D | 273 | 34.328 | −9.089 | 19.580 | 1.00 | 38.60 | C |
| ATOM | 5704 | CD | ARG | D | 273 | 35.611 | −9.815 | 19.937 | 1.00 | 39.50 | C |
| ATOM | 5705 | NE | ARG | D | 273 | 35.893 | −9.742 | 21.371 | 1.00 | 43.20 | N |
| ATOM | 5706 | CZ | ARG | D | 273 | 35.236 | −10.397 | 22.334 | 1.00 | 45.03 | C |
| ATOM | 5707 | NH1 | ARG | D | 273 | 34.224 | −11.222 | 22.065 | 1.00 | 45.26 | N |
| ATOM | 5708 | NH2 | ARG | D | 273 | 35.604 | −10.217 | 23.604 | 1.00 | 45.36 | N |
| ATOM | 5709 | C | ARG | D | 273 | 32.660 | −6.866 | 18.511 | 1.00 | 35.37 | C |
| ATOM | 5710 | O | ARG | D | 273 | 33.433 | −5.907 | 18.472 | 1.00 | 34.32 | O |
| ATOM | 5711 | N | VAL | D | 274 | 31.561 | −6.900 | 19.263 | 1.00 | 35.97 | N |
| ATOM | 5712 | CA | VAL | D | 274 | 31.240 | −5.868 | 20.235 | 1.00 | 35.86 | C |
| ATOM | 5713 | CB | VAL | D | 274 | 29.723 | −5.637 | 20.327 | 1.00 | 35.69 | C |
| ATOM | 5714 | CG1 | VAL | D | 274 | 29.398 | −4.492 | 21.294 | 1.00 | 35.67 | C |
| ATOM | 5715 | CG2 | VAL | D | 274 | 29.175 | −5.312 | 18.939 | 1.00 | 35.44 | C |
| ATOM | 5716 | C | VAL | D | 274 | 31.825 | −6.320 | 21.572 | 1.00 | 35.57 | C |
| ATOM | 5717 | O | VAL | D | 274 | 31.568 | −7.442 | 22.008 | 1.00 | 33.30 | O |
| ATOM | 5718 | N | CYS | D | 275 | 32.618 | −5.449 | 22.202 | 1.00 | 35.65 | N |
| ATOM | 5719 | CA | CYS | D | 275 | 33.352 | −5.788 | 23.426 | 1.00 | 34.94 | C |
| ATOM | 5720 | CB | CYS | D | 275 | 34.613 | −6.586 | 23.064 | 1.00 | 35.34 | C |
| ATOM | 5721 | SG | CYS | D | 275 | 35.685 | −5.805 | 21.824 | 1.00 | 35.72 | S |
| ATOM | 5722 | C | CYS | D | 275 | 33.725 | −4.537 | 24.235 | 1.00 | 36.18 | C |
| ATOM | 5723 | O | CYS | D | 275 | 33.593 | −3.396 | 23.747 | 1.00 | 35.07 | O |
| ATOM | 5724 | N | ALA | D | 276 | 34.206 | −4.764 | 25.458 | 1.00 | 36.75 | N |
| ATOM | 5725 | CA | ALA | D | 276 | 34.541 | −3.683 | 26.387 | 1.00 | 37.95 | C |
| ATOM | 5726 | CB | ALA | D | 276 | 34.719 | −4.246 | 27.791 | 1.00 | 38.85 | C |
| ATOM | 5727 | C | ALA | D | 276 | 35.791 | −2.888 | 25.989 | 1.00 | 39.42 | C |
| ATOM | 5728 | O | ALA | D | 276 | 35.789 | −1.654 | 26.043 | 1.00 | 40.43 | O |
| ATOM | 5729 | N | CYS | D | 277 | 36.850 | −3.604 | 25.602 | 1.00 | 39.32 | N |
| ATOM | 5730 | CA | CYS | D | 277 | 38.141 | −2.998 | 25.264 | 1.00 | 39.66 | C |
| ATOM | 5731 | CB | CYS | D | 277 | 39.195 | −3.373 | 26.322 | 1.00 | 40.47 | C |
| ATOM | 5732 | SG | CYS | D | 277 | 40.603 | −2.246 | 26.336 | 1.00 | 43.65 | S |
| ATOM | 5733 | C | CYS | D | 277 | 38.604 | −3.430 | 23.861 | 1.00 | 38.15 | C |
| ATOM | 5734 | O | CYS | D | 277 | 39.386 | −4.373 | 23.742 | 1.00 | 37.16 | O |
| ATOM | 5735 | N | PRO | D | 278 | 38.119 | −2.749 | 22.800 | 1.00 | 36.80 | N |
| ATOM | 5736 | CA | PRO | D | 278 | 38.487 | −3.070 | 21.404 | 1.00 | 36.46 | C |
| ATOM | 5737 | CB | PRO | D | 278 | 37.882 | −1.915 | 20.603 | 1.00 | 36.64 | C |
| ATOM | 5738 | CG | PRO | D | 278 | 36.723 | −1.472 | 21.404 | 1.00 | 36.69 | C |
| ATOM | 5739 | CD | PRO | D | 278 | 37.051 | −1.728 | 22.857 | 1.00 | 36.48 | C |
| ATOM | 5740 | C | PRO | D | 278 | 39.991 | −3.155 | 21.123 | 1.00 | 36.04 | C |
| ATOM | 5741 | O | PRO | D | 278 | 40.448 | −4.121 | 20.504 | 1.00 | 33.87 | O |
| ATOM | 5742 | N | GLY | D | 279 | 40.739 | −2.148 | 21.583 | 1.00 | 35.95 | N |
| ATOM | 5743 | CA | GLY | D | 279 | 42.184 | −2.112 | 21.417 | 1.00 | 35.94 | C |
| ATOM | 5744 | C | GLY | D | 279 | 42.868 | −3.361 | 21.936 | 1.00 | 36.09 | C |
| ATOM | 5745 | O | GLY | D | 279 | 43.605 | −4.014 | 21.205 | 1.00 | 35.46 | O |
| ATOM | 5746 | N | ARG | D | 280 | 42.591 | −3.699 | 23.183 | 1.00 | 37.96 | N |
| ATOM | 5747 | CA | ARG | D | 280 | 43.213 | −4.859 | 23.829 | 1.00 | 40.05 | C |
| ATOM | 5748 | CB | ARG | D | 280 | 42.894 | −4.911 | 25.341 | 1.00 | 42.89 | C |
| ATOM | 5749 | CG | ARG | D | 280 | 43.928 | −5.770 | 26.068 | 1.00 | 45.57 | C |
| ATOM | 5750 | CD | ARG | D | 280 | 43.624 | −5.817 | 27.580 | 1.00 | 47.91 | C |
| ATOM | 5751 | NE | ARG | D | 280 | 42.748 | −6.941 | 27.883 | 1.00 | 49.01 | N |
| ATOM | 5752 | CZ | ARG | D | 280 | 41.511 | −6.890 | 28.404 | 1.00 | 50.34 | C |
| ATOM | 5753 | NH1 | ARG | D | 280 | 40.912 | −5.726 | 28.677 | 1.00 | 49.57 | N |
| ATOM | 5754 | NH2 | ARG | D | 280 | 40.860 | −8.039 | 28.629 | 1.00 | 50.00 | N |
| ATOM | 5755 | C | ARG | D | 280 | 42.792 | −6.166 | 23.169 | 1.00 | 39.24 | C |
| ATOM | 5756 | O | ARG | D | 280 | 43.630 | −7.016 | 22.871 | 1.00 | 39.58 | O |
| ATOM | 5757 | N | ASP | D | 281 | 41.495 | −6.314 | 22.916 | 1.00 | 39.80 | N |
| ATOM | 5758 | CA | ASP | D | 281 | 40.961 | −7.513 | 22.249 | 1.00 | 40.19 | C |
| ATOM | 5759 | CB | ASP | D | 281 | 39.424 | −7.526 | 22.262 | 1.00 | 41.91 | C |
| ATOM | 5760 | CG | ASP | D | 281 | 38.837 | −7.854 | 23.646 | 1.00 | 44.64 | C |
| ATOM | 5761 | OD1 | ASP | D | 281 | 39.584 | −8.246 | 24.572 | 1.00 | 45.91 | O |
| ATOM | 5762 | OD2 | ASP | D | 281 | 37.602 | −7.727 | 23.808 | 1.00 | 48.01 | O |
| ATOM | 5763 | C | ASP | D | 281 | 41.482 | −7.689 | 20.816 | 1.00 | 38.06 | C |
| ATOM | 5764 | O | ASP | D | 281 | 41.706 | −8.822 | 20.386 | 1.00 | 37.75 | O |
| ATOM | 5765 | N | ARG | D | 282 | 41.681 | −6.590 | 20.086 | 1.00 | 35.29 | N |
| ATOM | 5766 | CA | ARG | D | 282 | 42.331 | −6.669 | 18.769 | 1.00 | 34.34 | C |
| ATOM | 5767 | CB | ARG | D | 282 | 42.281 | −5.332 | 18.010 | 1.00 | 32.25 | C |
| ATOM | 5768 | CG | ARG | D | 282 | 43.049 | −5.360 | 16.697 | 1.00 | 31.05 | C |
| ATOM | 5769 | CD | ARG | D | 282 | 42.951 | −4.084 | 15.874 | 1.00 | 29.57 | C |
| ATOM | 5770 | NE | ARG | D | 282 | 44.013 | −4.068 | 14.865 | 1.00 | 28.64 | N |
| ATOM | 5771 | CZ | ARG | D | 282 | 44.503 | −2.985 | 14.259 | 1.00 | 27.80 | C |
| ATOM | 5772 | NH1 | ARG | D | 282 | 44.039 | −1.759 | 14.513 | 1.00 | 27.82 | N |
| ATOM | 5773 | NH2 | ARG | D | 282 | 45.477 | −3.134 | 13.375 | 1.00 | 26.84 | N |
| ATOM | 5774 | C | ARG | D | 282 | 43.787 | −7.129 | 18.928 | 1.00 | 35.58 | C |
| ATOM | 5775 | O | ARG | D | 282 | 44.218 | −8.070 | 18.261 | 1.00 | 34.89 | O |
| ATOM | 5776 | N | ARG | D | 283 | 44.526 | −6.466 | 19.819 | 1.00 | 36.17 | N |
| ATOM | 5777 | CA | ARG | D | 283 | 45.943 | −6.793 | 20.048 | 1.00 | 37.16 | C |
| ATOM | 5778 | CB | ARG | D | 283 | 46.579 | −5.824 | 21.061 | 1.00 | 36.69 | C |
| ATOM | 5779 | CG | ARG | D | 283 | 48.099 | −5.782 | 21.014 | 1.00 | 35.90 | C |
| ATOM | 5780 | CD | ARG | D | 283 | 48.655 | −4.870 | 22.100 | 1.00 | 35.95 | C |
| ATOM | 5781 | NE | ARG | D | 283 | 48.308 | −3.457 | 21.883 | 1.00 | 36.43 | N |
| ATOM | 5782 | CZ | ARG | D | 283 | 48.566 | −2.466 | 22.740 | 1.00 | 34.87 | C |
| ATOM | 5783 | NH1 | ARG | D | 283 | 48.204 | −1.224 | 22.431 | 1.00 | 32.97 | N |
| ATOM | 5784 | NH2 | ARG | D | 283 | 49.160 | −2.705 | 23.913 | 1.00 | 35.68 | N |
| ATOM | 5785 | C | ARG | D | 283 | 46.148 | −8.245 | 20.499 | 1.00 | 37.38 | C |
| ATOM | 5786 | O | ARG | D | 283 | 47.118 | −8.875 | 20.100 | 1.00 | 38.17 | O |
| ATOM | 5787 | N | THR | D | 284 | 45.223 | −8.767 | 21.306 | 1.00 | 38.18 | N |
| ATOM | 5788 | CA | THR | D | 284 | 45.266 | −10.159 | 21.768 | 1.00 | 39.54 | C |
| ATOM | 5789 | CB | THR | D | 284 | 44.267 | −10.399 | 22.918 | 1.00 | 40.21 | C |
| ATOM | 5790 | OG1 | THR | D | 284 | 44.517 | −9.450 | 23.965 | 1.00 | 40.44 | O |
| ATOM | 5791 | CG2 | THR | D | 284 | 44.389 | −11.823 | 23.483 | 1.00 | 39.38 | C |
| ATOM | 5792 | C | THR | D | 284 | 44.981 | −11.156 | 20.650 | 1.00 | 40.91 | C |
| ATOM | 5793 | O | THR | D | 284 | 45.732 | −12.121 | 20.489 | 1.00 | 39.94 | O |
| ATOM | 5794 | N | GLU | D | 285 | 43.909 | −10.936 | 19.883 | 1.00 | 42.33 | N |
| ATOM | 5795 | CA | GLU | D | 285 | 43.581 | −11.829 | 18.756 | 1.00 | 43.79 | C |
| ATOM | 5796 | CB | GLU | D | 285 | 42.194 | −11.523 | 18.167 | 1.00 | 43.38 | C |
| ATOM | 5797 | CG | GLU | D | 285 | 41.042 | −11.917 | 19.066 | 1.00 | 44.31 | C |
| ATOM | 5798 | CD | GLU | D | 285 | 39.667 | −11.809 | 18.407 | 1.00 | 46.35 | C |
| ATOM | 5799 | OE1 | GLU | D | 285 | 39.546 | −11.880 | 17.160 | 1.00 | 46.74 | O |
| ATOM | 5800 | OE2 | GLU | D | 285 | 38.678 | −11.676 | 19.165 | 1.00 | 48.09 | O |
| ATOM | 5801 | C | GLU | D | 285 | 44.642 | −11.812 | 17.645 | 1.00 | 44.63 | C |
| ATOM | 5802 | O | GLU | D | 285 | 44.812 | −12.805 | 16.941 | 1.00 | 45.18 | O |
| ATOM | 5803 | N | GLU | D | 286 | 45.333 | −10.686 | 17.486 | 1.00 | 46.62 | N |
| ATOM | 5804 | CA | GLU | D | 286 | 46.421 | −10.565 | 16.500 | 1.00 | 48.20 | C |
| ATOM | 5805 | CB | GLU | D | 286 | 46.666 | −9.097 | 16.137 | 1.00 | 46.17 | C |
| ATOM | 5806 | CG | GLU | D | 286 | 45.580 | −8.532 | 15.234 | 1.00 | 45.61 | C |
| ATOM | 5807 | CD | GLU | D | 286 | 45.793 | −7.067 | 14.857 | 1.00 | 44.68 | C |
| ATOM | 5808 | OE1 | GLU | D | 286 | 46.764 | −6.438 | 15.336 | 1.00 | 41.43 | O |
| ATOM | 5809 | OE2 | GLU | D | 286 | 44.971 | −6.543 | 14.077 | 1.00 | 42.91 | O |
| ATOM | 5810 | C | GLU | D | 286 | 47.730 | −11.243 | 16.944 | 1.00 | 50.06 | C |
| ATOM | 5811 | O | GLU | D | 286 | 48.540 | −11.633 | 16.110 | 1.00 | 49.41 | O |
| ATOM | 5812 | N | GLU | D | 287 | 47.935 | −11.349 | 18.256 | 1.00 | 50.67 | N |
| ATOM | 5813 | CA | GLU | D | 287 | 49.050 | −12.097 | 18.847 | 1.00 | 53.96 | C |
| ATOM | 5814 | CB | GLU | D | 287 | 49.107 | −11.773 | 20.355 | 1.00 | 54.94 | C |
| ATOM | 5815 | CG | GLU | D | 287 | 50.187 | −12.454 | 21.173 | 1.00 | 56.54 | C |
| ATOM | 5816 | CD | GLU | D | 287 | 50.254 | −11.952 | 22.605 | 1.00 | 58.00 | C |
| ATOM | 5817 | OE1 | GLU | D | 287 | 49.310 | −11.286 | 23.083 | 1.00 | 60.22 | O |
| ATOM | 5818 | OE2 | GLU | D | 287 | 51.270 | −12.230 | 23.267 | 1.00 | 60.55 | O |
| ATOM | 5819 | C | GLU | D | 287 | 48.902 | −13.614 | 18.614 | 1.00 | 56.65 | C |
| ATOM | 5820 | O | GLU | D | 287 | 49.893 | −14.306 | 18.361 | 1.00 | 57.99 | O |
| ATOM | 5821 | N | ASN | D | 288 | 47.661 | −14.106 | 18.660 | 1.00 | 58.46 | N |
| ATOM | 5822 | CA | ASN | D | 288 | 47.323 | −15.474 | 18.217 | 1.00 | 59.60 | C |
| ATOM | 5823 | CB | ASN | D | 288 | 45.905 | −15.817 | 18.705 | 1.00 | 61.27 | C |
| ATOM | 5824 | CG | ASN | D | 288 | 45.795 | −15.864 | 20.215 | 1.00 | 63.65 | C |
| ATOM | 5825 | OD1 | ASN | D | 288 | 46.774 | −16.080 | 20.938 | 1.00 | 64.45 | O |
| ATOM | 5826 | ND2 | ASN | D | 288 | 44.585 | −15.627 | 20.711 | 1.00 | 62.14 | N |
| ATOM | 5827 | C | ASN | D | 288 | 47.444 | −15.663 | 16.683 | 1.00 | 60.10 | C |
| ATOM | 5828 | O | ASN | D | 288 | 46.549 | −16.175 | 15.986 | 1.00 | 60.54 | O |
| TER | 5829 | | ASN | D | 288 | | | | | | |
| HETATM | 5830 | ZN | ZN | E | 1 | −8.302 | 4.929 | −47.968 | 1.00 | 22.64 | ZN |
| HETATM | 5831 | ZN | ZN | E | 2 | 25.938 | −3.610 | −16.620 | 1.00 | 30.64 | ZN |
| HETATM | 5832 | AS | ARS | F | 1 | 0.861 | −32.847 | −4.106 | 1.00 | 58.05 | AS |
| HETATM | 5833 | AS | ARS | F | 2 | 33.397 | −0.120 | 15.432 | 1.00 | 56.64 | AS |
| HETATM | 5834 | O2 | EDO | G | 1 | 11.288 | −8.133 | −32.956 | 1.00 | 40.13 | O |
| HETATM | 5835 | C2 | EDO | G | 1 | 11.305 | −8.295 | −31.529 | 1.00 | 38.55 | C |
| HETATM | 5836 | C1 | EDO | G | 1 | 12.034 | −7.105 | −30.919 | 1.00 | 38.98 | C |
| HETATM | 5837 | O1 | EDO | G | 1 | 11.257 | −6.043 | −30.375 | 1.00 | 38.97 | O |
| HETATM | 5838 | O | HOH | H | 1 | 10.546 | −34.152 | −14.326 | 1.00 | 14.38 | O |
| HETATM | 5839 | O | HOH | H | 2 | −6.936 | 1.089 | −27.916 | 1.00 | 21.90 | O |
| HETATM | 5840 | O | HOH | H | 3 | 23.174 | −6.265 | −26.423 | 1.00 | 9.86 | O |
| HETATM | 5841 | O | HOH | H | 4 | 24.623 | −5.157 | −28.626 | 1.00 | 16.14 | O |
| HETATM | 5842 | O | HOH | H | 5 | 23.293 | −3.234 | −30.508 | 1.00 | 22.40 | O |
| HETATM | 5843 | O | HOH | H | 6 | 27.538 | −0.733 | −36.925 | 1.00 | 15.03 | O |
| HETATM | 5844 | O | HOH | H | 7 | 24.704 | −7.686 | −29.706 | 1.00 | 9.78 | O |
| HETATM | 5845 | O | HOH | H | 8 | 23.094 | −9.469 | −28.654 | 1.00 | 11.38 | O |
| HETATM | 5846 | O | HOH | H | 9 | 24.377 | −11.801 | −28.778 | 1.00 | 12.76 | O |
| HETATM | 5847 | O | HOH | H | 10 | 26.794 | −15.308 | −27.424 | 1.00 | 13.35 | O |
| HETATM | 5848 | O | HOH | H | 11 | −11.794 | −4.930 | −34.064 | 1.00 | 6.03 | O |
| HETATM | 5849 | O | HOH | H | 12 | −12.848 | 0.042 | −39.933 | 1.00 | 11.14 | O |
| HETATM | 5850 | O | HOH | H | 13 | −18.476 | 3.613 | −39.435 | 1.00 | 12.68 | O |
| HETATM | 5851 | O | HOH | H | 14 | −11.140 | 7.007 | −38.083 | 1.00 | 13.66 | O |
| HETATM | 5852 | O | HOH | H | 15 | 23.602 | 0.817 | 5.287 | 1.00 | 27.74 | O |
| HETATM | 5853 | O | HOH | H | 16 | 2.220 | 12.294 | −26.797 | 1.00 | 13.65 | O |
| HETATM | 5854 | O | HOH | H | 17 | −4.775 | 0.703 | −49.909 | 1.00 | 19.89 | O |
| HETATM | 5855 | O | HOH | H | 18 | −1.909 | −1.759 | −40.244 | 1.00 | 13.81 | O |
| HETATM | 5856 | O | HOH | H | 19 | 7.813 | −40.700 | −17.977 | 1.00 | 14.57 | O |
| HETATM | 5857 | O | HOH | H | 20 | 11.310 | −42.554 | −16.609 | 1.00 | 20.90 | O |
| HETATM | 5858 | O | HOH | H | 21 | −13.708 | −0.516 | −17.025 | 1.00 | 24.93 | O |
| HETATM | 5859 | O | HOH | H | 22 | 37.764 | −12.766 | −48.601 | 1.00 | 30.29 | O |
| HETATM | 5860 | O | HOH | H | 23 | 34.914 | −6.319 | −18.962 | 1.00 | 25.70 | O |
| HETATM | 5861 | O | HOH | H | 24 | −9.846 | 5.557 | −35.936 | 1.00 | 10.61 | O |
| HETATM | 5862 | O | HOH | H | 25 | 44.646 | 7.542 | −29.772 | 1.00 | 25.22 | O |
| HETATM | 5863 | O | HOH | H | 26 | 41.416 | −6.080 | −40.098 | 1.00 | 27.10 | O |
| HETATM | 5864 | O | HOH | H | 27 | 14.428 | −5.610 | −40.341 | 1.00 | 17.88 | O |
| HETATM | 5865 | O | HOH | H | 28 | 17.296 | 4.198 | −34.356 | 1.00 | 13.22 | O |
| HETATM | 5866 | O | HOH | H | 29 | 17.857 | −24.839 | −16.541 | 1.00 | 26.55 | O |
| HETATM | 5867 | O | HOH | H | 30 | 30.497 | −5.581 | −21.416 | 1.00 | 39.01 | O |
| HETATM | 5868 | O | HOH | H | 31 | 4.105 | 16.135 | −25.467 | 1.00 | 26.30 | O |
| HETATM | 5869 | O | HOH | H | 32 | −9.764 | 12.418 | −35.541 | 1.00 | 12.52 | O |
| HETATM | 5870 | O | HOH | H | 33 | −12.254 | 9.842 | −26.165 | 1.00 | 33.18 | O |
| HETATM | 5871 | O | HOH | H | 34 | 49.159 | 6.045 | −24.243 | 1.00 | 11.23 | O |
| HETATM | 5872 | O | HOH | H | 35 | 46.715 | 8.022 | −26.649 | 1.00 | 19.15 | O |
| HETATM | 5873 | O | HOH | H | 36 | 15.713 | −3.071 | −25.116 | 1.00 | 18.79 | O |
| HETATM | 5874 | O | HOH | H | 37 | 28.009 | −13.022 | −38.141 | 1.00 | 16.83 | O |
| HETATM | 5875 | O | HOH | H | 38 | 4.389 | −2.476 | −17.939 | 1.00 | 24.17 | O |
| HETATM | 5876 | O | HOH | H | 39 | −10.966 | 3.998 | −34.069 | 1.00 | 15.68 | O |
| HETATM | 5877 | O | HOH | H | 40 | 10.606 | −27.389 | −16.719 | 1.00 | 24.34 | O |
| HETATM | 5878 | O | HOH | H | 41 | 14.454 | −0.708 | −35.638 | 1.00 | 10.96 | O |
| HETATM | 5879 | O | HOH | H | 42 | 32.579 | 2.561 | −24.431 | 1.00 | 17.68 | O |
| HETATM | 5880 | O | HOH | H | 43 | 8.861 | −3.080 | −45.814 | 1.00 | 22.36 | O |
| HETATM | 5881 | O | HOH | H | 44 | −9.327 | −36.116 | −16.030 | 1.00 | 37.89 | O |
| HETATM | 5882 | O | HOH | H | 45 | 5.519 | −33.536 | −1.708 | 1.00 | 29.53 | O |
| HETATM | 5883 | O | HOH | H | 46 | 2.732 | −9.435 | −21.846 | 1.00 | 16.72 | O |
| HETATM | 5884 | O | HOH | H | 47 | 10.129 | −7.313 | −35.399 | 1.00 | 23.47 | O |
| HETATM | 5885 | O | HOH | H | 48 | −12.063 | 6.983 | −52.138 | 1.00 | 28.26 | O |
| HETATM | 5886 | O | HOH | H | 49 | 6.049 | −2.372 | −32.628 | 1.00 | 19.61 | O |
| HETATM | 5887 | O | HOH | H | 50 | −3.457 | −6.394 | −25.128 | 1.00 | 26.55 | O |
| HETATM | 5888 | O | HOH | H | 51 | −1.954 | −32.313 | −31.055 | 1.00 | 31.16 | O |
| HETATM | 5889 | O | HOH | H | 52 | 15.703 | −18.098 | −18.860 | 1.00 | 43.59 | O |
| HETATM | 5890 | O | HOH | H | 53 | 20.716 | 0.989 | −47.669 | 1.00 | 19.24 | O |
| HETATM | 5891 | O | HOH | H | 54 | 6.266 | −28.691 | −30.400 | 1.00 | 13.01 | O |
| HETATM | 5892 | O | HOH | H | 55 | −17.390 | −2.282 | −23.653 | 1.00 | 24.91 | O |
| HETATM | 5893 | O | HOH | H | 56 | −7.967 | 16.380 | −25.296 | 1.00 | 19.95 | O |
| HETATM | 5894 | O | HOH | H | 57 | 27.602 | −7.956 | −37.266 | 1.00 | 22.72 | O |
| HETATM | 5895 | O | HOH | H | 58 | −7.649 | 15.815 | −36.762 | 1.00 | 20.13 | O |
| HETATM | 5896 | O | HOH | H | 59 | 20.369 | −8.715 | −34.583 | 1.00 | 10.89 | O |
| HETATM | 5897 | O | HOH | H | 60 | 21.176 | −4.239 | −45.154 | 1.00 | 21.49 | O |
| HETATM | 5898 | O | HOH | H | 61 | 22.612 | 3.024 | −9.187 | 1.00 | 29.92 | O |
| HETATM | 5899 | O | HOH | H | 62 | 36.043 | 2.799 | −45.889 | 1.00 | 22.18 | O |
| HETATM | 5900 | O | HOH | H | 63 | 6.530 | 9.204 | −21.796 | 1.00 | 29.22 | O |
| HETATM | 5901 | O | HOH | H | 64 | −12.917 | 9.069 | −21.793 | 1.00 | 30.45 | O |
| HETATM | 5902 | O | HOH | H | 65 | 32.007 | 9.076 | −35.836 | 1.00 | 46.17 | O |
| HETATM | 5903 | O | HOH | H | 66 | 4.259 | −30.671 | −27.625 | 1.00 | 17.73 | O |
| HETATM | 5904 | O | HOH | H | 67 | 11.647 | −17.204 | −9.332 | 1.00 | 59.33 | O |
| HETATM | 5905 | O | HOH | H | 68 | 55.598 | −12.840 | −17.564 | 1.00 | 43.60 | O |
| HETATM | 5906 | O | HOH | H | 69 | −2.393 | −10.441 | −18.108 | 1.00 | 38.00 | O |
| HETATM | 5907 | O | HOH | H | 70 | 17.327 | 7.819 | −19.821 | 1.00 | 28.39 | O |
| HETATM | 5908 | O | HOH | H | 71 | −6.917 | −32.528 | −14.298 | 1.00 | 25.35 | O |
| HETATM | 5909 | O | HOH | H | 72 | 36.118 | −12.388 | −37.785 | 1.00 | 17.56 | O |
| HETATM | 5910 | O | HOH | H | 73 | 20.027 | −7.722 | −7.568 | 1.00 | 18.51 | O |
| HETATM | 5911 | O | HOH | H | 74 | −13.901 | 8.927 | −29.990 | 1.00 | 12.05 | O |
| HETATM | 5912 | O | HOH | H | 75 | 48.721 | 1.232 | −29.216 | 1.00 | 21.23 | O |
| HETATM | 5913 | O | HOH | H | 76 | −9.540 | 8.222 | −34.960 | 1.00 | 10.32 | O |
| HETATM | 5914 | O | HOH | H | 77 | 21.375 | −13.298 | −35.135 | 1.00 | 19.59 | O |
| HETATM | 5915 | O | HOH | H | 78 | 23.203 | 9.227 | 3.095 | 1.00 | 27.71 | O |
| HETATM | 5916 | O | HOH | H | 79 | 15.101 | −25.766 | −29.894 | 1.00 | 24.97 | O |
| HETATM | 5917 | O | HOH | H | 80 | 45.012 | −8.013 | −44.705 | 1.00 | 45.68 | O |
| HETATM | 5918 | O | HOH | H | 81 | −3.113 | 7.285 | −9.950 | 1.00 | 42.55 | O |
| HETATM | 5919 | O | HOH | H | 82 | 2.719 | −44.518 | −27.762 | 1.00 | 31.54 | O |
| HETATM | 5920 | O | HOH | H | 83 | −5.130 | −34.046 | −7.055 | 1.00 | 40.04 | O |
| HETATM | 5921 | O | HOH | H | 84 | −10.595 | −36.561 | −0.314 | 1.00 | 24.60 | O |
| HETATM | 5922 | O | HOH | H | 85 | −1.793 | −24.012 | −16.904 | 1.00 | 27.71 | O |
| HETATM | 5923 | O | HOH | H | 86 | 50.531 | −1.159 | −30.176 | 1.00 | 15.04 | O |
| HETATM | 5924 | O | HOH | H | 87 | −6.447 | 8.476 | −26.941 | 1.00 | 25.03 | O |
| HETATM | 5925 | O | HOH | H | 88 | 1.755 | −47.003 | −23.734 | 1.00 | 39.87 | O |
| HETATM | 5926 | O | HOH | H | 89 | 9.132 | −1.821 | −34.930 | 1.00 | 35.10 | O |
| HETATM | 5927 | O | HOH | H | 90 | 22.121 | −28.910 | −3.928 | 1.00 | 26.85 | O |
| HETATM | 5928 | O | HOH | H | 91 | 36.633 | −1.148 | −11.488 | 1.00 | 34.42 | O |
| HETATM | 5929 | O | HOH | H | 92 | −1.726 | 9.928 | −13.456 | 1.00 | 22.39 | O |
| HETATM | 5930 | O | HOH | H | 93 | 7.593 | −41.426 | −10.964 | 1.00 | 24.09 | O |
| HETATM | 5931 | O | HOH | H | 94 | 38.475 | 9.159 | −34.219 | 1.00 | 29.17 | O |
| HETATM | 5932 | O | HOH | H | 95 | −13.479 | 19.353 | −34.904 | 1.00 | 24.63 | O |
| HETATM | 5933 | O | HOH | H | 96 | 46.681 | 0.481 | 26.036 | 1.00 | 30.99 | O |
| HETATM | 5934 | O | HOH | H | 97 | −1.623 | −39.086 | −23.338 | 1.00 | 39.93 | O |
| HETATM | 5935 | O | HOH | H | 98 | 34.135 | 1.739 | −17.318 | 1.00 | 34.18 | O |
| HETATM | 5936 | O | HOH | H | 99 | −3.762 | 6.316 | −43.067 | 1.00 | 24.86 | O |
| HETATM | 5937 | O | HOH | H | 100 | 14.520 | −9.435 | −45.305 | 1.00 | 42.82 | O |
| HETATM | 5938 | O | HOH | H | 101 | 6.164 | −15.208 | −5.382 | 1.00 | 55.08 | O |
| HETATM | 5939 | O | HOH | H | 102 | 6.672 | −43.984 | −11.216 | 1.00 | 28.19 | O |
| HETATM | 5940 | O | HOH | H | 103 | 29.884 | 4.329 | −42.317 | 1.00 | 19.65 | O |
| HETATM | 5941 | O | HOH | H | 104 | 18.244 | −37.981 | −2.119 | 1.00 | 56.26 | O |
| HETATM | 5942 | O | HOH | H | 105 | 7.281 | 6.674 | −24.802 | 1.00 | 19.04 | O |
| HETATM | 5943 | O | HOH | H | 106 | −12.941 | −2.374 | −18.742 | 1.00 | 24.53 | O |
| HETATM | 5944 | O | HOH | H | 107 | 16.965 | −39.044 | −23.805 | 1.00 | 26.90 | O |
| HETATM | 5945 | O | HOH | H | 108 | 13.392 | −3.145 | −23.881 | 1.00 | 24.76 | O |
| HETATM | 5946 | O | HOH | H | 109 | 39.344 | −13.085 | −46.473 | 1.00 | 27.23 | O |
| HETATM | 5947 | O | HOH | H | 110 | 0.179 | 7.177 | −44.820 | 1.00 | 27.97 | O |
| HETATM | 5948 | O | HOH | H | 111 | −12.239 | 17.441 | −31.278 | 1.00 | 39.02 | O |
| HETATM | 5949 | O | HOH | H | 112 | 23.450 | 5.998 | −15.165 | 1.00 | 17.31 | O |
| HETATM | 5950 | O | HOH | H | 113 | 21.910 | 0.640 | −24.957 | 1.00 | 36.89 | O |
| HETATM | 5951 | O | HOH | H | 114 | 24.549 | 6.514 | −25.784 | 1.00 | 23.89 | O |
| HETATM | 5952 | O | HOH | H | 115 | 22.585 | 5.348 | −31.089 | 1.00 | 15.54 | O |
| HETATM | 5953 | O | HOH | H | 116 | 18.702 | −4.279 | −39.256 | 1.00 | 24.02 | O |
| HETATM | 5954 | O | HOH | H | 117 | 17.092 | −5.617 | −41.155 | 1.00 | 21.14 | O |
| HETATM | 5955 | O | HOH | H | 118 | 17.255 | 3.063 | −41.207 | 1.00 | 13.75 | O |
| HETATM | 5956 | O | HOH | H | 119 | 19.338 | 3.926 | −41.934 | 1.00 | 21.58 | O |
| HETATM | 5957 | O | HOH | H | 120 | 18.489 | −4.611 | −45.486 | 1.00 | 18.63 | O |
| HETATM | 5958 | O | HOH | H | 121 | 10.280 | −2.144 | −27.276 | 1.00 | 13.12 | O |
| HETATM | 5959 | O | HOH | H | 122 | 5.791 | −6.636 | −35.857 | 1.00 | 21.77 | O |
| HETATM | 5960 | O | HOH | H | 123 | −5.359 | −33.218 | 4.149 | 1.00 | 23.97 | O |
| HETATM | 5961 | O | HOH | H | 124 | 10.486 | −23.213 | −19.191 | 1.00 | 35.47 | O |
| HETATM | 5962 | O | HOH | H | 125 | 13.978 | −20.881 | −22.310 | 1.00 | 34.73 | O |
| HETATM | 5963 | O | HOH | H | 126 | −15.499 | 5.280 | −21.820 | 1.00 | 16.89 | O |
| HETATM | 5964 | O | HOH | H | 127 | −15.654 | 5.332 | −19.187 | 1.00 | 26.12 | O |
| HETATM | 5965 | O | HOH | H | 128 | −15.937 | −6.507 | −28.635 | 1.00 | 23.77 | O |
| HETATM | 5966 | O | HOH | H | 129 | −10.129 | −7.733 | −31.726 | 1.00 | 19.16 | O |
| HETATM | 5967 | O | HOH | H | 130 | −4.604 | −4.014 | −22.614 | 1.00 | 20.19 | O |
| HETATM | 5968 | O | HOH | H | 131 | 1.801 | −2.711 | −18.768 | 1.00 | 18.56 | O |
| HETATM | 5969 | O | HOH | H | 132 | 29.810 | 7.285 | −35.608 | 1.00 | 21.35 | O |
| HETATM | 5970 | O | HOH | H | 133 | 26.512 | 6.367 | −41.635 | 1.00 | 37.03 | O |
| HETATM | 5971 | O | HOH | H | 134 | 15.616 | 6.260 | −35.579 | 1.00 | 17.44 | O |
| HETATM | 5972 | O | HOH | H | 135 | 43.425 | 3.834 | −18.950 | 1.00 | 20.37 | O |
| HETATM | 5973 | O | HOH | H | 136 | 40.838 | 5.060 | −16.193 | 1.00 | 42.14 | O |
| HETATM | 5974 | O | HOH | H | 137 | 31.142 | 6.164 | −17.048 | 1.00 | 46.54 | O |
| HETATM | 5975 | O | HOH | H | 138 | 18.805 | 5.969 | −43.724 | 1.00 | 26.54 | O |
| HETATM | 5976 | O | HOH | H | 139 | 43.605 | 2.196 | −29.791 | 1.00 | 41.57 | O |
| HETATM | 5977 | O | HOH | H | 140 | 40.468 | 2.779 | −31.709 | 1.00 | 21.51 | O |
| HETATM | 5978 | O | HOH | H | 141 | 40.498 | −0.304 | −7.212 | 1.00 | 37.50 | O |
| HETATM | 5979 | O | HOH | H | 142 | 37.334 | −9.317 | −20.513 | 1.00 | 24.40 | O |
| HETATM | 5980 | O | HOH | H | 143 | −3.845 | 17.248 | −21.362 | 1.00 | 21.87 | O |
| HETATM | 5981 | O | HOH | H | 144 | 19.295 | −9.692 | 10.739 | 1.00 | 27.12 | O |
| HETATM | 5982 | O | HOH | H | 145 | 33.084 | −6.084 | −20.690 | 1.00 | 36.38 | O |
| HETATM | 5983 | O | HOH | H | 146 | 0.159 | 7.130 | −15.757 | 1.00 | 19.30 | O |
| HETATM | 5984 | O | HOH | H | 147 | 1.823 | −39.428 | −22.185 | 1.00 | 17.88 | O |
| HETATM | 5985 | O | HOH | H | 148 | 4.057 | 8.583 | −21.693 | 1.00 | 29.33 | O |
| HETATM | 5986 | O | HOH | H | 149 | −11.097 | 9.993 | −35.800 | 1.00 | 11.08 | O |
| HETATM | 5987 | O | HOH | H | 150 | 8.301 | −17.240 | −15.678 | 1.00 | 82.01 | O |
| HETATM | 5988 | O | HOH | H | 151 | 27.374 | 3.123 | −15.565 | 1.00 | 36.88 | O |
| HETATM | 5989 | O | HOH | H | 152 | 14.583 | −29.965 | −9.097 | 1.00 | 27.81 | O |
| HETATM | 5990 | O | HOH | H | 153 | 13.104 | 0.486 | −13.499 | 1.00 | 43.77 | O |
| HETATM | 5991 | O | HOH | H | 154 | 19.123 | −6.511 | −21.016 | 1.00 | 28.19 | O |
| HETATM | 5992 | O | HOH | H | 155 | 7.190 | 8.937 | −14.819 | 1.00 | 34.00 | O |
| HETATM | 5993 | O | HOH | H | 156 | −15.485 | 4.636 | −25.542 | 1.00 | 33.31 | O |
| HETATM | 5994 | O | HOH | H | 157 | 34.285 | −7.075 | −48.971 | 1.00 | 32.08 | O |
| HETATM | 5995 | O | HOH | H | 158 | 20.631 | −38.196 | −7.044 | 1.00 | 31.56 | O |
| HETATM | 5996 | O | HOH | H | 159 | 21.181 | −38.932 | −17.252 | 1.00 | 28.96 | O |
| HETATM | 5997 | O | HOH | H | 160 | −3.847 | −25.516 | −27.099 | 1.00 | 43.70 | O |
| HETATM | 5998 | O | HOH | H | 161 | −15.201 | 7.363 | −43.579 | 1.00 | 30.53 | O |
| HETATM | 5999 | O | HOH | H | 162 | −9.608 | −3.785 | −48.044 | 1.00 | 42.07 | O |
| HETATM | 6000 | O | HOH | H | 163 | 33.566 | −24.520 | −19.735 | 1.00 | 45.78 | O |
| HETATM | 6001 | O | HOH | H | 164 | 20.295 | −10.640 | −36.501 | 1.00 | 21.52 | O |
| HETATM | 6002 | O | HOH | H | 165 | 15.677 | 7.328 | 2.264 | 1.00 | 26.69 | O |
| HETATM | 6003 | O | HOH | H | 166 | 24.876 | −4.928 | −11.428 | 1.00 | 25.12 | O |
| HETATM | 6004 | O | HOH | H | 167 | 35.718 | 7.770 | −6.534 | 1.00 | 25.18 | O |
| HETATM | 6005 | O | HOH | H | 168 | 41.505 | 3.554 | −29.045 | 1.00 | 46.79 | O |
| HETATM | 6006 | O | HOH | H | 169 | 15.002 | −23.770 | −8.876 | 1.00 | 33.82 | O |
| HETATM | 6007 | O | HOH | H | 170 | −12.296 | 14.378 | −49.848 | 1.00 | 36.64 | O |
| HETATM | 6008 | O | HOH | H | 171 | 20.059 | −16.046 | −24.142 | 1.00 | 18.15 | O |
| HETATM | 6009 | O | HOH | H | 172 | −9.390 | 9.533 | 45.742 | 1.00 | 22.03 | O |
| HETATM | 6010 | O | HOH | H | 173 | −8.638 | −39.190 | −2.680 | 1.00 | 19.71 | O |
| HETATM | 6011 | O | HOH | H | 174 | −18.795 | −1.408 | −34.625 | 1.00 | 30.08 | O |
| HETATM | 6012 | O | HOH | H | 175 | −18.319 | 5.865 | −37.794 | 1.00 | 23.83 | O |
| HETATM | 6013 | O | HOH | H | 176 | −13.514 | 18.515 | −42.733 | 1.00 | 35.10 | O |
| HETATM | 6014 | O | HOH | H | 177 | 13.137 | −27.262 | −20.142 | 1.00 | 26.93 | O |
| HETATM | 6015 | O | HOH | H | 178 | 22.166 | −9.792 | −37.930 | 1.00 | 20.73 | O |
| HETATM | 6016 | O | HOH | H | 179 | −7.766 | −48.240 | −19.476 | 1.00 | 42.26 | O |
| HETATM | 6017 | O | HOH | H | 180 | 38.211 | −15.840 | −38.458 | 1.00 | 36.94 | O |
| HETATM | 6018 | O | HOH | H | 181 | 24.221 | 8.436 | 18.450 | 1.00 | 48.28 | O |
| HETATM | 6019 | O | HOH | H | 182 | −0.152 | −1.489 | −47.379 | 1.00 | 24.76 | O |
| HETATM | 6020 | O | HOH | H | 183 | −12.961 | 13.761 | −29.303 | 1.00 | 16.73 | O |
| HETATM | 6021 | O | HOH | H | 184 | −6.667 | 10.217 | −43.922 | 1.00 | 36.72 | O |
| HETATM | 6022 | O | HOH | H | 185 | 16.720 | −31.568 | −14.794 | 1.00 | 43.91 | O |
| HETATM | 6023 | O | HOH | H | 186 | 48.869 | 1.310 | 23.231 | 1.00 | 27.69 | O |
| HETATM | 6024 | O | HOH | H | 187 | 35.689 | −8.519 | −7.174 | 1.00 | 45.82 | O |
| HETATM | 6025 | O | HOH | H | 188 | 35.702 | −17.526 | −30.005 | 1.00 | 43.94 | O |
| HETATM | 6026 | O | HOH | H | 189 | −4.864 | −8.415 | −40.340 | 1.00 | 26.51 | O |
| HETATM | 6027 | O | HOH | H | 190 | 21.197 | −30.148 | −19.216 | 1.00 | 26.85 | O |
| HETATM | 6028 | O | HOH | H | 191 | −5.634 | −29.575 | −16.175 | 1.00 | 33.25 | O |

END

1-70. (canceled)

71. A tertiary structure (“PANDA Core”) formed on a p53 comprising a PANDA Pocket, a PANDA Agent, and at least one tight association between the PANDA Pocket and the PANDA Agent, wherein:
the PANDA Pocket is a region consisting essentially of an area of about 7 Å
from a properly folded PANDA Cysteine, including, all amino acids adjacent to one or more properly folded PANDA Cysteine, all amino acids that contact with one or more properly folded PANDA Cysteine, and all PANDA Cysteines;
the PANDA Agent is a composition of matter that has one or more useful characteristics as follows:
(a) can cause a substantial increase in the population of properly folded p53, preferably the increase is at least about 3 times more than the increase caused by PRIMA-1, more preferably the increase is at least about 5 times more than the increase caused by PRIMA-1, further preferably the increase is at least about 10 times more than the increase caused by PRIMA-1, further preferably the increase is at least about 100 times more than the increase caused by PRIMA-1;
(b) can cause a substantial improvement in the transcription function of p53, preferably the improvement is at least about 3 times more than the improvement caused by PRIMA-1; more preferably the improvement is at least about 5 times more than the improvement caused by PRIMA-1, further preferably the improvement is at least about 10 times more than the improvement caused by PRIMA-1, further preferably the improvement is at least about 100 times than the improvement caused by PRIMA-1; and
(c) can cause a substantial enhancement of stabilization of p53 as measured by an increase p53 Tm, preferably the enhancement is at least about 3 times more than the enhancement caused by PRIMA-1, more preferably the improvement is at least about 5 times more than the improvement caused by PRIMA-1, further preferably the improvement is at least about 10 times more than the improvement caused by PRIMA-1, further preferably the improvement is at least about 100 times than the improvement caused by PRIMA-1;
wherein the PANDA Agent is preferably has two or more useful characteristics, more preferably has three or more useful characteristics; and
the PANDA Cysteine is a cysteine corresponding to the wtp53 positions cysteine 124 (“C124”), cysteine 135 (“C135”), and cysteine 141 (“C141”) (together the “PANDA Triad”).

72. The PANDA Core of claim 71, wherein the PANDA Pocket consists essentially of the PANDA Triad and the amino acids corresponding to wtp53 positions S116, C275, R273, Y234, V122, T123, T125, Y126, M133, F134, Q136, L137, K139, T140, P142, V143, L114, H115, G117, T118, A119, K120, S121, A138, 1232, H233, N235, Y236, M237, C238, N239, F270, E271, V272, V274, A276, C277, P278, G279, R280, D281, and R282.

73. The PANDA Core of claim 72, wherein the p53 is any wildtype p53 (“wtp53”), including all natural and artificial p53; any mutated p53 (“mp53”), including all natural and artificial p53; or a combination thereof.

74. The PANDA Core of claim 73, wherein:
the wtp53 is a p53α, p53β, p53γ, A40p53α, A40p53β, A40p53γ, or any of the preceding p53 with one or more single nucleotide polymorphism (“SNP”);
the mp53 has at least one mutation on p53, including any single amino acid mutation, preferably the mutation alters and/or partially alters the structure and/or function of p53, including one or more mutations corresponding to the wtp53 positions R175, G245, R248, R249, R273, R282, C176, H179, Y220, P278, V143, I232, and F270; and including one or more R175H, G245D/S, R248Q/W, R249S, R273C/H, R282W, C176F, H179R, Y220C, P278S, V143A, I232T, and F270C mutations; and/or
the artificial p53 includes any artificially engineered p53, including a p53 fusion protein, a p53 fragment, a p53 peptide, a p53-derived fusion macromolecule, a p53 recombinant protein, a p53 with second-site suppressor mutation (“SSSM”), and a super p53.

75. The PANDA Core of claim 71, wherein the tight association includes a bond, covalent bond, a non-covalent bond, and a combination thereof.

76. The PANDA Core of claim 75, wherein the tight association substantially stabilizes p53, preferably the Tm of p53 increases by at least about 0.5° C., preferably by at least about 1° C., more preferably by at least about 2° C., further preferably by at least about 5° C., further preferably by at least about 8° C.

77. The PANDA Core of claim 71, wherein the PANDA Agent includes one or more PANDA Pocket-binding group (“R”) capable of binding one or more amino acids on PANDA Pocket, preferably one or more cysteine, more preferably two or more cysteines, further preferably more than three cysteines, further preferably from about three cysteines to about 12 cysteines.

78. The PANDA Core of claim 77, wherein R is a metal, a metalloid, or a group including a Michael acceptor and a thiol group; preferably an arsenic, an antimony, a bismuth, any analogue of the foregoing, or a combination thereof.

79. The PANDA Core as in claim 78, wherein R contains a 3-valence and/or 5-valence arsenic atom, a 3-valence and/or 5-valence antimony atom, a 3-valence and/or 5-valence bismuth atom, and/or a combination thereof.

80. The PANDA Core as in claim 71, wherein the PANDA Agent is a compound or a combination of compounds selected from Table 1, and Table 2.

81. The PANDA Core as in claim 80, wherein the PANDA Agent is a compound or a combination of compounds selected from the group consisting of As2O3, As2O5, KAsO2, NaAsO2, HAsNa2O4, HAsK2O4, AsF3, AsCl3, AsBr3, AsI3, AsAc3, As(OC 2H5)3, As(OCH3)3, As2(SO4)3, (CH3CO2)3As, C8H4K2012As2.xH 2O, HOC6H4COOAsO, [O2CCH2C(OH)(CO2)CH2CO2]As, Sb2O3, Sb2O5, KSbO2, NaSbO2, HSbNa2O4, HSbK2O4, SbF3, SbCl3, SbBr3, SbI3, SbAc3, Sb(OC2H5)3, Sb(OCH3) 3, Sb2(SO4)3, (CH3CO2)3Sb, C8H4K2O12Sb2.xH2O, HOC6H4COOSbO, [O2CCH2C(OH)(CO 2)CH2CO2]Sb, Bi2O3, Bi2O5, KBiO2, NaBiO2, HBiNa2O4, HBiK2O4, BiF3, BiCl3, BiBr3, BiI3, BiAc3, Bi(OC 2H5)3, Bi(OCH3)3, Bi2(SO4)3, (CH3CO2)3Bi, C8H4K2O12Bi2.xH 2O, HOC6H4COOBiO, 16H18As2N4O2 (NSC92909), C13H14As2O6 (NSC48300), C10H13NO8Sb (NSC31660), C6H12NaO8Sb+ (NSC15609), C13H21NaO9Sb+ (NSC15623), and a combination thereof.

82. A complex (“PANDA”) comprising a p53 and the PANDA Core of claim 71.

83. The complex of claim 82, wherein said PANDA Core is purified and isolated.

84. The complex of claim 82, wherein, as compared to when the PANDA Agent is not bound, the PANDA Core or the complex has gained one or more wtp53 structure, preferably a DNA binding structure; has gained one or more wtp53 function, preferably a transcription function; and/or has lost and/or diminishes one or more mp53 function, preferably an oncogenic function.

85. The complex of claim 82, wherein The PANDA Core or the complex has gained any function in vitro and/or in vivo, including any wildtype function including molecule-level association to nucleic acids, transcriptional activation or repression of target genes, association to wtp53 or mp53 partners, dissociation to wtp53 or mp53 partners, and reception to post-translational modification; cell-level responsiveness to stresses including nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, invasion and metastasis; and organism-level delay or prevention of cancer relapse, increase of cancer treatment efficacy, increase of response ratio to cancer treatment, regulation of development, senescence, longevity, immunological processes, and aging.

86. The complex of claim 82, wherein the PANDA Core or the complex has lost, impaired and/or abrogated a function in vitro and/or in vivo, including any function promoting cancer cell metastasis, genomic instability, invasion, migration, scattering, angiogenesis, stem cell expansion, survival, proliferation, tissue remodelling, resistance to therapy, and mitogenic defects.

87. The complex of claim 82, wherein the PANDA Core or the complex has the ability to upregulate or downregulate one or more p53 downstream targets, at an RNA level and/or protein level, in a biological system, preferably by about 3 times, more preferably by about 5 times, further preferably by about 10-100 times.

88. The complex of claim 82, wherein the PANDA Core or the complex has the ability to treat a p53-relevant disease in a subject with mp53 and/or without functional p53, wherein the disease is a cancer, a tumor, a consequence of aging, a developmental disease, accelerated aging, an immunological disease, or a combination thereof.

89. A method of turning on and off a wtp53 function of a mp53, the method comprising the steps:
(a) combining a first PANDA Agent with the mp53 to turn on the wtp53 function of a mp53; and
(b) adding a second compound that (i) removes the PANDA Agent from the mp53, including British Anti-Lewisite (BAL), succimer (DMSA), Unithiol (DMPS), and/or a combination thereof; (ii) inhibits expression of p53 including doxycycline in engineered cells or subjects, and/or (iii) turning off p53 expression, including tamoxifen, in engineered cells or subjects.

90. A method of identifying PANDA or PANDA Core, the method comprising the step (s) of:
using an antibody specific for properly folded PANDA including PAb1620, PAb246, and/or PAb240, to perform immunoprecipitation;
measuring increase of molecular weight by mass spectroscopy;
measuring whether transcriptional activity is restored in a luciferase assay;
measuring the mRNA and protein levels of p53 targets;
co-crystalizing to construct 3-D structure; and/or
measuring increase of Tm.
